(19) United States
(12) Patent Application Publication
(10) Pub. No.: US 2026/0062700 A1
(43) Pub. Date: Mar. 5, 2026

(54) ENGINEERED RETRONS AND METHODS OF USE

(71) Applicant: ReNAgade Therapeutics Management Inc., Cambridge, MA (US)

(72) Inventors: Alim LADHA, Cambridge, MA (US); Vladimir PRESNYAK, Cambridge, MA (US); Inna SHCHERBAKOVA, Cambridge, MA (US); Brian GOODMAN, Cambridge, MA (US); Mario Rodriguez MESTRE, Cambridge, MA (US); Kyong-Rim KIEFFER-KWON, Cambridge, MA (US); Socheata LY, Cambridge, MA (US); Devin Scott QUINLAN, Cambridge, MA (US); Muthusamy JAYARAMAN, Cambridge, MA (US); Stephen SCULLY, Cambridge, MA (US); Jacob LAYER, Cambridge, MA (US); Abril Fleitas Beitans, Cambridge, MA (US); Zhongxia YI, Cambridge, MA (US)

(21) Appl. No.: 19/104,782

(22) PCT Filed: Aug. 24, 2023
(86) PCT No.: PCT/US2023/072872

§ 371 Date: Feb. 19, 2025

Related U.S. Application Data
(63) Continuation-in-part of application No. 18/087,673, filed on Dec. 22, 2022, now Pat. No. 11,866,728.
Application No. 18/087,673 is a continuation-in-part of application No. PCT/US2023/061038, filed on Jan. 20, 2023.
(60) Provisional application No. 63/373,545, filed on Aug. 25, 2022.
(60) Provisional application No. 63/476,900, filed on Dec. 22, 2022.
(60) Provisional application No. 63/488,317, filed on Mar. 3, 2023.
(60) Provisional application No. 63/491,603, filed on Mar. 22, 2023.
(60) Provisional application No. 63/515,783, filed on Jul. 26, 2023.

(51) Int. Cl. C12N 15/113; A61K 31/7105; A61K 48/00; C12N 5/071; C12N 5/10; C12N 9/12; C12N 9/22; C12N 15/62

(52) U.S. Cl. CPC C12N 15/113; A61K 31/7105; A61K 48/005; C12N 5/0602; C12N 5/10; C12N 9/1276; C12N 9/226; C12N 15/62; C12Y 207/07049; C12N 2310/20; C12N 2310/531; C12N 2510/00

(57) ABSTRACT

Disclosed are engineered retrons and methods of use such as to modify the genome of a host (e.g, mammalian) cell by delivering the engineered retron or the encoded ncRNA in vitro or in vivo to the host (e.g., mammalian) cell.

### RELATED APPLICATIONS

This application claims priority under 35 U.S.C. § 119(e) to U.S. Provisional Application Ser. No. 63/373,545, filed Aug. 25, 2022 (RTX003-P1), U.S. application Ser. No. 18/087,673, filed Dec. 22, 2022 (RTX004-T1), U.S. Provisional Application Ser. No. 63/476,900, filed Dec. 22, 2022 (RTX005-P1), International PCT Application No. PCT/US2023/61038, filed Jan. 20, 2023 (RTX006-PCT1), U.S. Provisional Application Ser. No. 63/488,317 (RTX007-P1), filed Mar. 3, 2023, U.S. Provisional Application Ser. No. 63/491,603, filed Mar. 22, 2023 (RTX008-P1), and U.S. Provisional Application Ser. No. 63/515,783, filed Jul. 26, 2023 (RTX009-P1), each of which are incorporated herein by reference in their entireties.

This application references the following applications: U.S. Provisional Application Ser. No. 63/301,936, filed Jan. 21, 2022 (RTX001-P1), and U.S. Provisional Application Ser. No. 63/370,880, filed Aug. 9, 2022 (RTX002-P1), each of which are incorporated herein by reference in their entireties.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

This application as originally filed includes/contains a Sequence Listing filed in electronic form in eXtensible Markup Language (XML) format entitled J0356-99004.xml, created on Aug. 24, 2023 and having a size of 36,884,841 bytes. The contents of the Sequence Listing are incorporated herein in its entirety.

### TECHNICAL FIELD

The present disclosure generally relates to systems, methods and compositions used for precise genome editing, including nucleic acid insertions, replacements, and deletions at targeted and precise genome sites, wherein said systems, methods, and compositions are based on novel and/or engineered retrons.

### BACKGROUND OF THE INVENTION

Precise genome editing by programmable nucleases (e.g., RNA-guided nucleases (e.g., CRISPR nucleases), zinc-finger nucleases (ZFN), and transcription activator-like effector nucleases (TALENS)) typically relies on homology-directed repair (HDR) and the presence of a donor DNA template at the site of a double-strand break (DSB) induced by the programmable nuclease. It is generally accepted that a limiting step for HDR-dependent precise genome editing is the delivery of donor DNA template to the nuclease-induced DSB (e.g., see Ling et al., “Improving the efficiency of precise genome editing with site-specific Cas9-oligonucleotide conjugates,” Science Advances, 2020, Vol. 6, No. 15, pp. 1-8). Various methods aimed at boosting the efficiency of HDR-dependent editing have been reported, many of which involve the physical tethering of the DNA donor to a component of the precise editing system. Exemplary methods have been discussed in: K. Lee et al., “Synthetically modified guide RNA and donor DNA are a versatile platform for CRISPR-Cas9 engineering,” eLife 6, e25312 (2017); J. Carlson-Stevermer et al., “Assembly of CRISPR ribonucleoproteins with biotinylated oligonucleotides via an RNA aptamer for precise gene editing,” Nat. Commun. 8, 1711 (2017); N. Savic, et al., “Covalent linkage of the DNA repair template to the CRISPR-Cas9 nuclease enhances homology-directed repair,” eLife 7, e33761 (2018); and E. J. Aird et al., “Increasing Cas9-mediated homology-directed repair efficiency through covalent tethering of DNA repair template.,” Commun. Biol. 1, 54 (2018), each of which are incorporated herein by reference. Despite these efforts, efficiency of HDR-dependent precise editing remains unsatisfactory.

Retrons are defined by their unique ability to produce an unusual satellite DNA known as msDNA (multicopy single-stranded DNA). DNA encoding retrons includes a reverse trancriptase (RT)-coding gene (ret) and a nucleic acid sequence encoding the non-coding RNA (ncRNA), which contains two contiguous and inverted non-coding sequences referred to as the msr and msd. The ret gene and the non-coding RNA (including the msr and msd) are transcribed as a single RNA transcript, which becomes folded into a specific secondary structure following post-transcriptional processing. Once translated, the RT binds the RNA template downstream from the msd locus, initiating reverse transcription of the RNA towards its 5′ end, assisted by the 2′OH group present in a conserved branching guanosine residue that acts as a primer. Reverse transcription halts before reaching the msr locus, and the resulting DNA, the msDNA, remains covalently attached to the RNA template via a 2′-5′ phosphodiester bond and base-pairing between the 3′ ends of the msDNA and the RNA template. The external regions, at the 5′ and 3′ ends of the msd/msr transcript (a1 and a2, respectively) are complementary and can hybridize, leaving the structures located in the msr and msd regions in internal positions (see FIG. 1A). The msr locus, which is not reverse transcribed, forms one to three short stem-loops of variable size, ranging from 3 to 10 base pairs, whereas the msd locus folds into a single/double long hairpin with a highly variable long stem of 10-50 bp in length that is also present in the final msDNA form.

It has recently been reported that retrons may be utilized as a means to provide donor DNA template for HDR-dependent genome editing (e.g., see Lopez et al., “Precise genome editing across kingdoms of life using retron-derived DNA,” Nature Chemical Biology, Dec. 12, 2021, 18, pages 199-206 (2022)), however, producing sufficient levels of donor DNA template intracellularly to sufficiently support efficient HDR-dependent editing remains a significant challenge. Improved retron-based genome modification systems are highly desirous in the art.

### SUMMARY OF THE INVENTION

In one aspect, the present disclosure provides recombinant retrons comprising one or more genetic modifications which improves the functionality and/or properties of a retron. Such genetic modifications can include a mutation, insertion, deletion, inversion, replacement, substitution, or translocation of one or more contiguous or non-contiguous nucleobases in a nucleic acid molecule encoding a retron or a component of a retron, such as an ncRNA or a reverse transcriptase. In various aspects, the retron that becomes modified with the one or more genetic modifications (i.e., the “pre-modified” or “unmodified” retron or retron component) is a naturally occurring retron or retron component (e.g., naturally occurring ncRNA of Table A or RT) ability to facilitate homology-dependent recombination (or HDR) in a cell, thereby resulting in a relative increase in the concentrations or amounts of msDNA comprising a DNA donor template. In particular embodiments, the recombinant retrons are based on and/or derived from a naturally-occurring retron, such as any retron-related sequence provided by Table X (the introduction of the one or more genetic modifications into a set of 7257 previously unknown retrons discovered through computational methods described herein (e.g., see Examples). In other embodiments, the recombinant retrons are based on introducing the one or more genetic modifications into previously available retron sequences (e.g., the “Mestre et al., Systematic Prediction of Genes Functionally Associated with Bacterial Retrons and Classification of The Encoded Tripartite Systems, Nucleic Acids Research, Volume 48, Issue 22, 16 Dec. 2020, Pages 12632-12647” (incorporated herein by reference) to achieve recombinant retrons with the enhanced ability to produce increased concentrations or amounts of msDNA comprising a DNA donor template.

In another aspect, the present disclosure further provides nucleic acid molecules encoding the recombinant retrons and/or recombinant retron components (e.g., a recombinant ncRNA and/or a recombinant retron RT). In still another aspect, the present disclosure provides genome editing systems comprising recombinant retron components (e.g., recombinant ncRNA and/or recombinant RT), programmable nucleases (e.g., RNA-guided nucleases, such as CRISPR-Cas proteins, ZFPs, and TALENS), and guide RNAs (in the case where RNA-guide nucleases are used in said genome editing systems). In a further aspect, the disclosure provides nucleic acid molecules encoding the described genome editing systems and said components thereof, as well as polypeptides making up the components of said genome editing systems. In yet another aspect, the disclosure provides vectors for transferring and/or expressing said genome editing systems, e.g., under in vitro, ex vivo, and in vivo conditions. In still another aspect, the disclosure provides cell-delivery compositions and methods, including compositions for passive and/or active transport to cells (e.g., plasmids), delivery by virus-based recombinant vectors (e.g., AAV and/or lentivirus vectors), delivery by non-virus-based systems (e.g., liposomes and LNPs), and delivery by virus-like particles. Depending on the delivery system employed, the retron-based genome editing systems described herein may be delivered in the form of DNA (e.g., plasmids or DNA-based virus vectors), RNA (e.g., ncRNA and mRNA delivered by LNPs), a mixture of DNA and RNA, protein (e.g., virus-like particles), and ribonucleoprotein (RNP) complexes. Any suitable combinations of approaches for delivering the components of the herein disclosed retron-based genome editing systems may be employed. In one embodiment, each of the components of the retron-based genome editing system is delivered by an all-RNA system, e.g., the delivery of one or more RNA molecules (e.g., mRNA and/or ncRNA) by one or more LNPs, wherein the one or more RNA molecules form the ncRNA and guide RNA (as needed) and/or are translated into the polypeptide components (e.g., the RT and a programmable nuclease). In yet another aspect, the disclosure provides methods for genome editing by introducing a retron-based genome editing system described herein into a cell (e.g., under in vitro, in vivo, or ex vivo conditions) comprising a target edit site, thereby resulting in an edit at the target edit. In other aspects, the disclosure provides formulations comprising any of the aforementioned components for delivery to cells and/or tissues, including in vitro, in vivo, and ex vivo delivery, recombinant cells and/or tissues modified by the recombinant retron-based genome modification systems and methods described herein, and methods of modifying cells by conducting genome editing and related DNA donor-dependent methods, such as recombineering, or cell recording, using the herein disclosed retron-based genome modification systems. The disclosure also provides methods of making the recombinant retrons, retron-based genome modification systems, vectors, compositions and formulations described herein, as well as to pharmaceutical compositions and kits for modifying cells under in vitro, in vivo, and ex vivo conditions that comprise the herein disclosed genome editing and/or modification systems.

In an embodiment, this disclosure or the inventions herein provide a gene editing system comprising one or more delivery vehicles, wherein: the delivery vehicle(s) comprise RNA cargo; the RNA cargo comprises (a) at least one mRNA molecule encoding (i) a nucleic acid programmable nuclease and (ii) a retron reverse transcriptase, (b) an engineered retron ncRNA, and (c) guide RNA for the programmable nuclease; and each delivery vehicle contains (a)(i) and/or (a)(ii) and/or (b) and/or (c); whereby one delivery vehicle or more than one delivery vehicle delivers (a)(i), (a)(ii), (b), and (c).

In an embodiment, in the gene editing system, (a)(i) and (a)(ii) comprise a single mRNA molecule encoding the nucleic acid programmable nuclease and the retron reverse transcriptase.

In an embodiment, in the gene editing system, (a)(i) and (a)(ii) are encoded and expressed as a fusion protein.

In an embodiment, in the gene editing system (a)(i) and (a)(ii) are encoded and expressed as a fusion protein and the fusion protein comprises the C-terminal end of the nucleic acid programmable nuclease fused to the N-terminal end of the retron reverse transcriptase (nuclease:RT fusion); or the fusion protein comprises the N-terminal end of the nucleic acid programmable nuclease fused to the C-terminal end of the retron reverse transcriptase (RT:nuclease fusion).

In an embodiment, in the gene editing system, (a)(i) and (a)(ii) comprise a first mRNA molecule encoding the nucleic acid programmable nuclease and a second mRNA molecule encoding the retron reverse transcriptase.

In an embodiment, in the gene editing system, (c) is separate from (a)(i), (a)(ii) and (b) or is provided in trans.

In an embodiment, in the gene editing system, (b) the engineered retron ncRNA, and (c) the guide RNA are fused or are provided in cis.

In an embodiment, in the gene editing system, (b) the engineered retron ncRNA, and (c) the guide RNA are fused or are provided in cis and the guide RNA is fused to the 5′ end of the retron ncRNA.

In an embodiment, in the gene editing system, (b) the engineered retron ncRNA, and (c) the guide RNA are fused or are provided in cis and the guide RNA is fused to the 3′ end of the retron ncRNA.

In an embodiment, in the gene editing system, (b) the engineered retron ncRNA, and (c) the guide RNA are fused or are provided in cis and the engineered ncRNA comprises a first guide RNA fused to the 5′ end of the retron ncRNA, and a second guide RNA fused to the 3′ end of the retron ncRNA, and the first and second guide RNAs target different sequences. Thus, on a broader scale, in an embodiment, in the gene editing system, (c) guide RNA for the programmable nuclease, can comprise one or more guides that target the same or different target sequences. Such guide RNA(s) in an embodiment, can be single guide RNA(s) or sgRNA(s); for instance, when the nucleic acid programmable nuclease comprises a Cas9.

In an embodiment, in the gene editing system, the one or more delivery vehicles comprise a liposome or a lipid nanoparticle (LNP).

In an embodiment, in the gene editing system, (a) the at least one mRNA molecule encoding (i) the nucleic acid programmable nuclease and (ii) the retron reverse transcriptase, and (b) the engineered retron ncRNA, are in the same delivery vehicle.

In an embodiment, in the gene editing system, (a) the at least one mRNA molecule encoding (i) the nucleic acid programmable nuclease and (ii) the retron reverse transcriptase, and (b) the engineered retron ncRNA, are in separate delivery vehicles.

In an embodiment, in the gene editing system, the nucleic acid programmable nuclease and the retron reverse transcriptase are encoded on separate mRNA molecules and those separate mRNA molecules of (a)(i) and (a)(ii) are contained in the same delivery vehicle.

In an embodiment, in the gene editing system, the nucleic acid programmable nuclease and the retron reverse transcriptase are encoded on separate mRNA molecules and those separate mRNA molecules of (a)(i) and (a)(ii) are contained in different delivery vehicles.

In an embodiment, in the gene editing system, the engineered retron ncRNA includes a sequence of interest encoding a donor polynucleotide comprising an intended edit to be integrated at a target sequence in a cell, and wherein the donor polynucleotide is flanked by a 5′ homology arm that hybridizes to a sequence 5′ to the target sequence and a 3′ homology arm that hybridizes to a sequence 3′ to the target sequence. In an embodiment, the donor polynucleotide can be heterologous to the cell. In an embodiment, the donor polynucleotide can be endogenous to the cell; for instance, the cell can contain a sequence that is typical for those in a population having a disease state and the donor polynucleotide can be a sequence that is typical for those in the population not having a non-disease state (e.g., the donor can be for a genetic correction or repair of a cell to modify the cell from having a mutation or modification that gives rise to a disease state to having a sequence typical of not having the disease state). Such can be done in an animal cell, or a mammalian cell (e.g., a primate, a non-human primate, or a domesticated mammal such as a cat or dog or horse) or a human cell; for instance to correct, address, treat, mitigate a genetic condition in the animal, mammal, domesticated mammal, cat, dog, horse or human. Such can be done in plant cells to introduce mutations that give rise to favorable phenotypic characteristics such as disease resistance or other favorable plant trait(s).

In an embodiment, in the gene editing system, the nucleic acid programmable nuclease comprises a Cas9 nuclease, a TnpB nuclease, or a Cas12a nuclease.

In an embodiment, in the gene editing system, the engineered retron ncRNA comprises: A) a pre-msr sequence having a first complementary region of the retron ncRNA; B) an msr sequence including an msr stem-loop structure; C) an msd sequence including an msd stem-loop structure and a sequence of interest, wherein said msd sequence templates a single strand DNA product (RT-DNA) in the presence of the retron reverse transcriptase; and D) a post-msd sequence having a second complementary region, wherein the first and second complementary regions form an a1/a2 duplex region of the retron ncRNA, wherein the msr stem-loop structure, the msd stem-loop structure, or the a1/a2 duplex comprise a modification which result in increased editing efficiency in the presence of a nucleic acid programmable nuclease that associates with the one or more guide RNAs, and wherein optionally one or more of the guide RNAs of (c) are coupled to the pre-msr sequence, the post-msd sequence, or both the pre-msr sequence and the post-msd sequence. In such an embodiment where the engineered retron ncRNA comprises A), B), C) and D), wherein the sequence of interest can encode a donor polynucleotide comprising an intended edit to be integrated at a target sequence of a cell, wherein the donor polynucleotide is flanked by a 5′ homology arm that hybridizes to a sequence 5′ to the target sequence and a 3′ homology arm that hybridizes to a sequence 3′ to the target sequence. In such an embodiment where the engineered retron ncRNA comprises A), B), C) and D) (either with the sequence of interest encoding a donor polynucleotide or simply being a sequence of interest), the ncRNA has a nucleotide sequence of Table B, or a nucleotide sequence having at least 75%, 80%, 85%, 90%, 95%, 99%, or 100% sequence identity with a sequence from Table B. The donor polynucleotide can be heterologous to a cell. Alternatively, the donor polynucleotide can be endogenous to the cell. For instance, the cell can contain a sequence that is typical for those in a population having a disease state and the donor polynucleotide can be a sequence that is typical for those in the population not having a non-disease state (e.g., the donor can be for a genetic correction or repair of a cell to modify the cell from having a mutation or modification that gives rise to a disease state to having a sequence typical of not having the disease state).

In an embodiment of the gene editing system, the gene editing system can comprise any combination(s) of the foregoing embodiments of the gene editing system.

In an embodiment, this disclosure or the inventions herein provide a cell, such as an isolated cell comprising the gene editing system disclosed herein, such as in any of the foregoing paragraphs. In an embodiment the cell, e.g., isolated cell, can be a eukaryotic cell. In an embodiment the eukaryotic cell can be a plant cell or an animal cell or a mammalian cell, e.g., an isolated plant cell or an isolated animal cell or an isolated mammalian cell. In an embodiment, the mammalian cell, e.g., an isolated mammalian cell, can be a human cell. In an embodiment, the cell can be a prokaryotic cell, e.g., a bacterial cell. In such an embodiment where the cell is a bacterial cell, the donor polynucleotide can code for antibiotic susceptibility; and thus, the invention can involve a means for addressing antibiotic resistant bacteria by rendering such bacteria susceptible to antibiotics (and a subject to whom the gene editing system is administered can also then receive antibiotics to which the bacteria are rendered susceptible by the gene editing system).

In an embodiment, this disclosure or the inventions herein provide a composition comprising: a) the gene editing system disclosed herein, such as in any of the foregoing paragraphs; and b) a pharmaceutically or veterinarily acceptable carrier. In an embodiment, in the composition the delivery vehicle can comprise a lipid nanoparticle comprising: a) one or more ionizable lipids; b) one or more structural lipids; c) one or more PEGylated lipids; and d) one or more phospholipids. In an embodiment, in the composition the one or more ionizable lipids comprises an ionizable lipid set forth in Table 2.

In an embodiment, this disclosure or the inventions herein provide uses of the gene editing system embodiments and/or the compositions disclosed herein, such as in any of the foregoing paragraphs; for instance, use in modifying a cell or genetically modifying a cell, e.g., a eukaryotic or a prokaryotic cell and/or an animal cell and/or a mammalian and/or a human cell and/or a bacterial cell and/or a plant cell, in vivo, in vitro or ex vivo (e.g., any cell discussed herein wherein the cell comprises an isolated cell). In an embodiment this disclosure or the inventions herein provide uses of the gene editing system embodiments and/or the compositions disclosed herein, such as in any of the foregoing paragraphs; for instance, use in treating or addressing a genetic condition of a subject,

In an embodiment, this disclosure or the inventions herein provide methods of genetically modifying a cell comprising: contacting a gene editing system as herein discussed, such as in any of the foregoing paragraphs, or a composition as herein discussed, such as in any of the foregoing paragraphs (which comprises a gene editing system as herein discussed, such as in any of the foregoing paragraphs), advantageously a gene editing system that includes a sequence of interest encoding a donor polynucleotide comprising an intended edit to be integrated at a target sequence in a cell, said method comprising contacting the composition or the gene editing system with the cell, thereby delivering the RNA cargo to the cell, wherein: the nucleic acid programmable nuclease forms a complex with the guide RNA, wherein said guide RNA directs the complex to the target sequence; the nucleic acid programmable nuclease creates a double-stranded break in in the target sequence; the retron reverse transcriptase and engineered retron ncRNA create RT DNA that comprises the donor polynucleotide; and the donor polynucleotide becomes integrated at the target sequence; whereby editing the cell is genetically modified. In an embodiment, the cell can be a eukaryotic or a prokaryotic cell or an animal cell or a mammalian cell or a human cell or a bacterial cell or a plant cell.

Exemplary and non-limiting aspects and embodiments of the disclosure are summarized as follows in the form of numbered paragraphs.
1. A gene editing system comprising one or more delivery vehicles, wherein:
   the delivery vehicle(s) comprise RNA cargo,
   said RNA cargo comprises (a) at least one mRNA molecule encoding (i) a nucleic acid programmable nuclease and (ii) a retron reverse transcriptase, (b) an engineered retron ncRNA, and (c) guide RNA for the nucleic acid programmable nuclease,
   each delivery vehicle contains (a)(i) and/or (a)(ii) and/or (b) and/or (c),
   whereby one delivery vehicle or more than one delivery vehicle delivers (a)(i), (a)(ii), (b), and (c).
2. The gene editing system of paragraph 1,
   wherein the engineered retron ncRNA comprises an HDR nucleotide sequence substituted into a retron ncRNA;
   wherein the retron reverse transcriptase has an amino acid sequence comprising at least 90% sequence identity to a retron reverse transcriptase of Table A;
   wherein the retron ncRNA has about 85% to 98% sequence identity to a retron ncRNA of Table B.
3. The gene editing system of paragraph 2, wherein the retron ncRNA and the retron reverse transcriptase are from the same clade.
4. The gene editing system of paragraph 2, wherein the retron ncRNA nucleotide sequence has about 85% to 98% sequence identity to SEQ ID NO:15327, and the retron reverse transcriptase has at least 90% sequence identity to a type I-C retron reverse transcriptase.
5. The gene editing system of paragraph 4, wherein the retron reverse transcriptase comprises an amino acid sequence at least about 90% identical to SEQ ID NO:1262.
6. The gene editing system of paragraph 4, wherein the retron ncRNA nucleotide sequence has about 85% to 98% sequence identity to SEQ ID NO:16411, and the retron reverse transcriptase has at least 90% sequence identity to a type III retron reverse transcriptase.
7. The gene editing system of paragraph 6, wherein the retron reverse transcriptase comprises an amino acid sequence at least about 90% identical to SEQ ID NO:2781.
8. The gene editing system of paragraph 6, wherein the retron ncRNA nucleotide sequence has about 85% to 98% sequence identity to SEQ ID NO:18731. and the retron reverse transcriptase has at least 90% sequence identity to a type XIII retron reverse transcriptase.
9. The gene editing system of paragraph 8, wherein the retron reverse transcriptase comprises an amino acid sequence at least about 90% identical to SEQ ID NO:6342.
10. The gene editing system of paragraph 1, wherein the retron reverse transcriptase comprises at least one amino acid substitution that increases processivity and/or fidelity.
11. The gene editing system of paragraph 10, wherein the retron reverse transcriptase comprises an amino acid substitution in an amino acid residue that corresponds to the following amino acid residues in Eco1 RT: Q190, E302, or T306.
12. The gene editing system of paragraph 10, wherein the retron reverse transcriptase comprises an amino acid substitution in an amino acid residue that corresponds to the following amino acid substitutions in Eco1 RT: Q190F, E302R, or T306K.
13. A gene editing system comprising one or more delivery vehicles, wherein:
   the delivery vehicle(s) comprise RNA cargo,
   said RNA cargo comprises (a) at least one mRNA molecule encoding (i) a nucleic acid programmable nuclease and (ii) an engineered retron reverse transcriptase, (b) an engineered retron ncRNA, and (c) guide RNA for the programmable nuclease,
   each delivery vehicle contains (a)(i) and/or (a)(ii) and/or (b) and/or (c),
   whereby one delivery vehicle or more than one delivery vehicle delivers (a)(i), (a)(ii), (b), and (c), and
   wherein the engineered retron reverse transcriptase comprises a processivity enhancing domain or a fidelity enhancing domain.
14. The gene editing system of paragraph 13, wherein the processivity enhancing domain comprises Sso7d or Sac7d.
15. The gene editing system of paragraph 13, wherein the fidelity enhancing domain comprises a 3′ to 5′ exonuclease domain.
16. The gene editing system of paragraph 15, wherein the exonuclease domain comprises POLE1 POLD1, POLG, Pfu, or KOD.
17. The gene editing system of paragraph 13, wherein the engineered retron ncRNA comprises an HDR nucleotide sequence substituted into a retron ncRNA;
   wherein the retron reverse transcriptase has an amino acid sequence comprising at least 90% sequence identity to a retron reverse transcriptase of Table A;
   wherein the retron ncRNA has about 85% to 98% sequence identity to a retron ncRNA of Table B.
18. The gene editing system of paragraph 13, wherein the retron ncRNA and the retron reverse transcriptase are from the same clade.
19. A gene editing system comprising one or more delivery vehicles, wherein:
   the delivery vehicle(s) comprise RNA cargo,
   said RNA cargo comprises (a) at least one mRNA molecule encoding (i) a nucleic acid programmable nuclease and (ii) an engineered reverse transcriptase, (b) an engineered retron ncRNA, and (c) guide RNA for the programmable nuclease,
   each delivery vehicle contains (a)(i) and/or (a)(ii) and/or (b) and/or (c),
   whereby one delivery vehicle or more than one delivery vehicle delivers (a)(i), (a)(ii), (b), and (c), and
   wherein the engineered reverse transcriptase comprises a Y region domain that is from a retron RT that corresponds to the engineered retron ncRNA.
20. The gene editing system of paragraph 19, wherein the engineered reverse transcriptase is a chimera comprising an MMLV RT fused to the Y region of the retron RT.
21. The gene editing system of paragraph 1, wherein (a)(i) and (a)(ii) comprise a single mRNA molecule encoding the nucleic acid programmable nuclease and the retron reverse transcriptase.
22. The gene editing system of paragraph 21, wherein (a)(i) and (a)(ii) are encoded and expressed as a fusion protein.
23. The gene editing system of paragraph 22, wherein the fusion protein comprises the C-terminal end of the nucleic acid programmable nuclease fused to the N-terminal end of the retron reverse transcriptase (nuclease:RT fusion).
24. The gene editing system of paragraph 22, wherein the fusion protein comprises the N-terminal end of the nucleic acid programmable nuclease fused to the C-terminal end of the retron reverse transcriptase (RT:nuclease fusion).
25. The gene editing system of paragraph 1, wherein (a)(i) and (a)(ii) comprise a first mRNA molecule encoding the nucleic acid programmable nuclease and a second mRNA molecule encoding the retron reverse transcriptase.
26. The gene editing system of paragraph 1, wherein (c) is separate from (a)(i), (a)(ii) and (b) or is provided in trans.
27. The gene editing system of paragraph 1, wherein (b) the engineered retron ncRNA, and (c) the guide RNA are fused or are provided in cis.
28. The gene editing system of paragraph 27, wherein the guide RNA is fused to the 5′ end of the retron ncRNA.
29. The gene editing system of paragraph 27, wherein the guide RNA is fused to the 3′ end of the retron ncRNA.
30. The gene editing system of paragraph 27, wherein the engineered ncRNA comprises a first guide RNA fused to the 5′ end of the retron ncRNA, and a second guide RNA fused to the 3′ end of the retron ncRNA, and the first and second guide RNAs target different sequences.
31. The gene editing system of paragraph 1, wherein the one or more delivery vehicles comprise a liposome or a lipid nanoparticle (LNP).
32. The gene editing system of paragraph 1, wherein (a) the at least one mRNA molecule encoding (i) the nucleic acid programmable nuclease and (ii) the retron reverse transcriptase, and (b) the engineered retron ncRNA are in the same delivery vehicle.
33. The gene editing system of paragraph 1, wherein the (a) the at least one mRNA molecule encoding (i) the nucleic acid programmable nuclease and (ii) the retron reverse transcriptase, and (b) the engineered retron ncRNA are in separate delivery vehicles.
34. The gene editing system of paragraph 1, wherein the nucleic acid programmable nuclease and the retron reverse transcriptase are encoded on separate mRNA molecules and those separate mRNA molecules of (a)(i) and (a)(ii) are contained in the same delivery vehicle.
35. The gene editing system of paragraph 1, wherein the nucleic acid programmable nuclease and the retron reverse transcriptase are encoded on separate mRNA molecules and those separate mRNA molecules of (a)(i) and (a)(ii) are contained in different delivery vehicles.
36. The gene editing system of paragraph 1, wherein the engineered retron ncRNA includes a sequence of interest encoding a donor polynucleotide comprising an intended edit to be integrated at a target sequence in a cell, and wherein the donor polynucleotide is flanked by a 5′ homology arm that hybridizes to a sequence 5′ to the target sequence and a 3′ homology arm that hybridizes to a sequence 3′ to the target sequence.
37. The gene editing system of paragraph 1, wherein the nucleic acid programmable nuclease comprises a Cas9 nuclease, a TnpB nuclease, or a Cas12a nuclease.
38. The gene editing system of paragraph 1, wherein the nucleic acid programmable nuclease comprises a Cas9 nuclease.
39. The gene editing system of paragraph 1, wherein the nucleic acid programmable nuclease comprises a Cas9 nickase.
40. An isolated cell comprising the gene editing system of paragraph 1.
41. The isolated cell of paragraph 40, wherein the isolated cell is a mammalian cell.
42. The isolated cell of paragraph 41, wherein the mammalian cell is a human cell.
43. A composition comprising:
   a) the gene editing system of paragraph 1; and
   b) a pharmaceutically or veterinarily acceptable carrier.
44. The composition of paragraph 43, wherein the delivery vehicle is a lipid nanoparticle comprising:
   a) one or more ionizable lipids;
   b) one or more structural lipids;
   c) one or more PEGylated lipids; and
   d) one or more phospholipids.
45. The composition of paragraph 44, wherein the one or more ionizable lipids comprises an ionizable lipid set forth in Table 2.
46. A method of genetically modifying a cell comprising:
contacting the gene editing system of paragraph 1 with the cell, thereby delivering the RNA cargo to the cell,
   wherein:
   the nucleic acid programmable nuclease forms a complex with the guide RNA, wherein said guide RNA directs the complex to the target sequence,
   the nucleic acid programmable nuclease creates a double-stranded break in in the target sequence,
   the retron reverse transcriptase and engineered retron ncRNA create RT DNA that comprises the donor polynucleotide, and
   the donor polynucleotide becomes integrated at the target sequence,
whereby editing the cell is genetically modified.

Further exemplary and non-limiting aspects and embodiments of the disclosure are summarized as follows in the form of numbered paragraphs.
1. An engineered nucleic acid construct comprising:
   a) a first polynucleotide encoding a non-coding RNA (ncRNA), said first polynucleotide comprising:
      1) an msr locus encoding the msr RNA portion of a multi-copy single-stranded DNA (msDNA); and
      2) an msd locus encoding the msd RNA portion of the msDNA; and
   b) one or more heterologous nucleic acids inserted at or within a location selected from: the msd locus, upstream of the msr locus, upstream of the msd locus, and downstream of the msd locus,
   wherein the ncRNA comprises:
   (I) an ncRNA listed in Table B, or an ncRNA having at least 50%, at least 55%, at least 60%, at least 65%, at least 70%, at least 75%, at least 80%, at least 85%, at least 90%, at least 910%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity with an ncRNA listed in Table B; and/or
   (II) an ncRNA having a conserved structure of any one of the ncRNA structures of FIGS. 2-27; and
   wherein the ncRNA optionally excludes any ncRNA associated in nature with any one of the retron reverse transcriptases of Table X.
2. The engineered nucleic acid construct of paragraph 1, further comprising a second polynucleotide encoding a reverse transcriptase (RT), or a portion thereof, wherein the encoded RT or portion thereof is capable of synthesizing a DNA copy of at least a portion of the msd locus encoding the msDNA.
3. The engineered nucleic acid construct of paragraph 2,
   wherein the second polynucleotide comprises:
      III) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide listed in Table A; and/or
      IV) encodes a consensus amino acid sequence of Table C, or encodes an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an amino acid sequence listed in Table C; and/or
   wherein the second polynucleotide encodes:
      V) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or
      VI) a polypeptide comprising a polypeptide consensus sequence listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an amino acid sequence listed in Table C; and/or
   wherein the second polynucleotide optionally does not encode an amino acid sequence listed in Table X.
4. An engineered nucleic acid construct comprising:
   a) a first polynucleotide encoding a non-coding RNA (ncRNA), said first polynucleotide comprising:
      1) an msr locus encoding the msr RNA portion of a multi-copy single-stranded DNA (msDNA); and
      2) an msd locus encoding the msd RNA portion of the msDNA;
   b) one or more heterologous nucleic acids inserted at or within a location selected from: the msd locus, upstream of the msr locus, upstream of the msd locus, and downstream of the msd locus; and
   c) a second polynucleotide encoding a reverse transcriptase (RT), or a portion thereof, wherein the encoded RT or portion thereof is capable of synthesizing a DNA copy of at least a portion of the msd locus encoding the msDNA, and,
   wherein the non-coding RNA (ncRNA) of the first polynucleotide optionally has a conserved structure of any one of the ncRNA structures of FIGS. 2-27;
   wherein the second polynucleotide comprises:
   I) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide listed in Table A; and/or
   wherein the second polynucleotide encodes:
   II) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or
   IV) a polypeptide comprising a polypeptide consensus sequence listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table C; and
   wherein the second polynucleotide optionally does not encode an amino acid sequence of Table X.
4a. An engineered nucleic acid construct, comprising:
   1) an msr locus (that encodes the msr RNA portion of an msDNA);
   2) an msd locus encoding the msd RNA portion of the msDNA;
   3) a sequence encoding a retron reverse transcriptase (RT), wherein said msd RNA is capable of being reverse transcribed to form the msDNA by the retron reverse transcriptase (RT); and,
   4) a heterologous nucleic acid inserted at or within the msd locus, upstream of the msr locus, upstream or downstream of the msd locus;
   wherein the engineered nucleic acid construct optionally has (a) a secondary structure of a wild-type ncRNA of any one of FIGS. 2-27 or
   b) a variant of a), having:
      i) up to 1, 2, or 3 (e.g., up to 1) nucleotide changes per 10 red lettered-nucleotides;
      ii) up to 4, 5, or 6 (e.g., up to 1 or 2) nucleotide changes per 10 black lettered-nucleotides; and/or
      iii) up to 7, 8, or 9 (e.g., up to 3 or 4) nucleotide changes per 10 grey lettered-nucleotides; and/or
      optionally further comprising:
      i) 7, 8, 9, or 10 (e.g., 9 or 10) nucleotides present per 10 red-circled nucleotides;
      ii) 6, 7, 8, 9, or 10 (e.g., 8, 9 or 10) nucleotides present per 10 black-circled nucleotides;
      iii) 4, 5, 6, 7, 8, 9, or 10 (e.g., 6, 7, 8, 9 or 10) nucleotides present per 10 grey-circled nucleotides; and/or
      iv) 2, 3, 4, 5, 6, 7, 8, 9, or 10 (e.g., 4, 5, 6, 7, 8, 9, or 10) nucleotides present per 10 white-circled nucleotides.
5. The engineered nucleic acid construct of any one of paragraphs 1 to 4a, comprising one or more sequence modifications (e.g., an insertion, deletion, and/or substitution of one or more nucleotide(s)) in the msr locus and/or the msd locus that:
   a) modulates (e.g., enhances) reverse transcription, processivity, accuracy/fidelity, and/or production of the msDNA (e.g., in the mammalian cell);
   b) modulates (e.g., reduces) immunogenicity of ncRNA encoded by the engineered retron (e.g., the msr locus and/or the msd locus) in a host (e.g., a host comprising the mammalian cell);
   c) modulates (e.g., inhibits, either permanently or transiently) a function of the msDNA; and/or
   d) modulates (e.g., improves) efficiency of targeted genome editing/engineering.
6. The engineered nucleic acid construct of any one of paragraphs 1 to 4, wherein said engineered nucleic acid construct has a secondary structure of a wild-type retron encoding a wild-type retron ncRNA encompassed by:
   a) any one of the structures as depicted in FIGS. 2-27, or
   b) a variant of a), having:
      i) up to 1, 2, or 3 (e.g., up to 1) nucleotide changes per 10 red lettered-nucleotides;
      ii) up to 4, 5, or 6 (e.g., up to 1 or 2) nucleotide changes per 10 black lettered-nucleotides; and/or
      iii) up to 7, 8, or 9 (e.g., up to 3 or 4) nucleotide changes per 10 grey lettered-nucleotides; and/or
   optionally further comprising:
      i) 7, 8, 9, or 10 (e.g., 9 or 10) nucleotides present per 10 red-circled nucleotides;
      ii) 6, 7, 8, 9, or 10 (e.g., 8, 9 or 10) nucleotides present per 10 black-circled nucleotides;
      iii) 4, 5, 6, 7, 8, 9, or 10 (e.g., 6, 7, 8, 9 or 10) nucleotides present per 10 grey-circled nucleotides; and/or
      iv) 2, 3, 4, 5, 6, 7, 8, 9, or 10 (e.g., 4, 5, 6, 7, 8, 9, or 10) nucleotides present per 10 white-circled nucleotides.
7. The engineered nucleic acid construct of any one of paragraphs 1-6, wherein the nucleic acid construct is engineered by introducing the one or more sequence modifications into a wild-type retron encoding a wild-type ncRNA listed in Table B.
8. The engineered nucleic acid construct of any one of paragraphs 1-7, wherein the one or more sequence modifications in the ncRNA comprises one or more of:
   (i) a modified (e.g., mutated, reduced, or eliminated) bulge in a1, a2, or both a1 and a2;
   (ii) an extension or shortening of a1, a2, or both a1 and a2;
   (iii) an extension or shortening of a spacer sequence between hairpin loops (e.g., S1, S2, S3, and/or S4);
   (iv) an additional or modified (e.g., mutated or eliminated) bulge in hairpin loops (e.g., L2 and/or L3 (e.g., by removing unpaired bases in the bulge, or by replacing unpaired bases with an equivalent number of base pairs));
   (v) a modified (e.g., extended or shortened) length of hairpin loops (e.g., L1, L2, L3, and/or L4);
   (vi) an alternative L1 and/or L2 having complement, reverse, or reverse complement sequences;
   (vii) a modified (e.g., increased) number of unpaired bases at the tip of hairpin loops (e.g., L1, L2, L3, and/or L4);
   (viii) a modified (e.g., increased or decreased) GC content in hairpin loops (e.g., L1, L2, L3, and/or L4);
   (ix) an insertion of the heterologous nucleic acid in spacer sequences between hairpin loops (e.g., S1, S2, S3 and/or S4), or at the tip of hairpin loops (e.g., L1, L2, L3, and/or L4);
   (x) a deletion of one or more hairpin loops (e.g., L1, L2, L3 and/or L4);
   (xi) an addition of a new loop in a spacer sequence between hairpin loops (e.g., S1, S2, S3, and/or S4);
   (xii) circularization of the ncRNA with the 5′ end and the 3′ end of the ncRNA being connected either directly, or via a spacer sequence;
   (xiii) a repositioned branching guanosine capable of initiating reverse transcription priming;
   (xiv) a staggered end sequence that reduces immunogenicity of the retron ncRNA, created by, e.g., adding or removing the 5′ al nucleotides and/or the 3′ a2 nucleotides; and/or,
   (xv) an antisense sequence complementary to a CRISPR/Cas guide RNA (gRNA) sequence encoded by the heterologous nucleic acid, wherein the antisense sequence hybridizes to and inhibits said gRNA in the encoded retron ncRNA, and wherein said antisense sequence is removed upon reverse transcription of the msDNA.
9. The engineered nucleic acid construct of any one of paragraphs 1-8, wherein the one or more heterologous nucleic acid sequences comprise:
   a) a heterologous nucleic acid (such as the coding sequence for an RNA aptamer or a ribozyme) inserted into the msr locus or the msd locus (such as in an S region (e.g., S1, S2, S3 and/or S4), or the tip of an L region (e.g., L1, L2, L3 and/or L4), or upstream or downstream of either the msr locus or the msd locus; or
   b) a first heterologous nucleic acid inserted into the msd locus, and a second heterologous nucleic acid inserted either upstream of the msr locus or downstream of the msd locus, wherein the second heterologous nucleic acid encodes a guide RNA.
10. The engineered nucleic acid construct of any one of paragraphs 1-9, wherein said heterologous nucleic acid encodes:
   (a) a protein or peptide of interest, or wherein said heterologous nucleic acid comprises;
   (b) a DNA donor template sequence;
   (c) a functional DNA element selected from a promoter, an enhancer, a protein binding sequence, a methylation site, a homology region for assisting gene editing, and the like; or
   (d) a coding sequence for a functional RNA element selected from a guide RNA and a ncRNA.
11. The engineered nucleic acid construct of paragraph 10, wherein said protein or peptide of interest comprises a therapeutic protein useful in treating a disease.
12. The engineered nucleic acid construct of paragraph 10, wherein said DNA donor template sequence corrects/repairs/removes a mutation at the target genome site.
13. The engineered nucleic acid construct of any one of paragraphs 1-12, further comprising or encoding a sequence-specific nuclease (such as a CRISPR/Cas effector enzyme, a ZFN, a TALEN, a meganuclease, TnpB, IscB, or a restriction endonuclease (RE)), and/or a DNA-repair modulating biomolecule.
13b. The engineered nucleic acid construct of paragraphs 1-13 wherein the engineered nucleic acid is an all-RNA component system.
13c. The engineered nucleic acid construct of paragraphs 1-13 wherein the engineered nucleic acid is an all-DNA molecule system.
14. The engineered nucleic acid construct of paragraph 13, wherein the sequence-specific nuclease is fused to the RT, optionally via a flexible linker (e.g., a flexible linker comprising Gly and Ser rich sequences such as G4S repeats or GS repeats) or by a generally disordered protein sequence (such as unstructured hydrophilic, biodegradable protein polymer, e.g., an XTEN peptide polymer).
15. The engineered nucleic acid construct of paragraph 13 or 14, wherein the nuclease is a CRISPR/Cas effector enzyme that forms a complex with a guide RNA (gRNA) recognizing a target sequence, wherein the gRNA is linked to the ncRNA and/or the msDNA, either directly or through a linker/spacer polynucleotide.
16. The engineered nucleic acid construct of paragraph 13, wherein the DNA-repair modulating biomolecule is a regulatory protein that modulates (e.g., enhances) HDR, and the regulatory protein is fused to the RT or to the sequence-specific nuclease, optionally via a flexible linker (e.g., the flexible linker comprising Gly and Ser rich sequences such as G4S repeats or GS repeats) or by a generally disordered protein sequence (such as unstructured hydrophilic, biodegradable protein polymer, e.g., an XTEN peptide polymer).
17. A vector system comprising one or more vectors comprising the engineered nucleic acid construct of any one of paragraphs 1-16, wherein the vector system is optionally all-RNA.
18. The vector system of paragraph 17, wherein the msr locus, the msd locus, and the polynucleotide encoding the RT are comprised within the same vector.
19. The vector system of paragraph 17 or 18, wherein the same vector further comprises a promoter operably linked to the msr locus and/or the msd locus.
20. The vector system of paragraph 19, wherein the promoter is further operably linked to the polynucleotide encoding the RT.
21. A vector system comprising one or more vectors, comprising the engineered nucleic acid construct of paragraph 1 or 2, wherein the vector system further comprises a second polynucleotide encoding a reverse transcriptase (RT), or a portion thereof, wherein the encoded RT is capable of synthesizing a DNA copy of at least a portion of the msd locus encoding the msDNA, and wherein the msr locus, the msd locus, and the second polynucleotide encoding the RT are provided by at least two different vectors.
22. The vector system of paragraph 21, wherein:
   a) the second polynucleotide comprises:
      i) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide listed in Table A; and/or
   b) the second polynucleotide encodes:
      i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or
      ii) a polypeptide listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table C; and
   wherein the second polynucleotide optionally does not encode a polypeptide listed in Table X.
23. The vector system of paragraph 21 or 22, wherein the polynucleotide encoding the RT is provided in trans with respect to the msr gene and/or the msd gene.
24. The vector system of any one of paragraphs 17-23, wherein the one or more vectors comprise a viral vector.
25. The vector system of paragraph 24, wherein the viral vector is a retroviral vector, a lentiviral vector, an adenoviral vector, an adeno-associated viral vector, a vaccinia viral vector, a poxviral vector, or a herpes simplex viral vector.
26. The vector system of any one of paragraphs 17-23, wherein the one or more vectors comprise a non-viral vector.
27. The vector system of paragraph 26, wherein the non-viral vector comprises a plasmid.
28. The vector system of paragraph 26, wherein the non-viral vector comprises a liposome, a lipid nanoparticle (LNP), a cationic polymer, a vesicle, or a gold nanoparticle.
29. The vector system of any one of paragraphs 17-28, comprising a vector encoding a sequence-specific nuclease.
30. The vector system of paragraph 29, wherein the sequence-specific nuclease comprises an RNA-guided sequence-specific nuclease (e.g., a CRISPR/Cas effector enzyme, an engineered RNA-guided FokI-nuclease (e.g., dCas-FokI), an RNA-guided DNA endonuclease, TnpB, IscB, or a transposon-associated nuclease), or a non-RNA-guided sequence-specific nuclease (e.g., a meganuclease, a zinc finger nuclease (ZFN), a TALE nuclease (TALEN), or a restriction endonuclease (RE)).
31. The vector system of paragraph 30, wherein the Cas effector enzyme is a Class 1, Type I, II, or III Cas; a Class 2, Type II Cas (e.g., Cas9); or a Class 2, Type V Cas (e.g., Cpf1).
32. The vector system of paragraph 30, wherein:
   1) the RNA-guided sequence-specific nuclease comprises the CRISPR/Cas effector enzyme, the engineered RNA-guided FokI-nuclease (e.g., dCas-FokI), the RNA-guided DNA endonuclease, TnpB, IscB, IsrB, or the transposon-associated nuclease; or,
   2) non-RNA-guided sequence-specific nuclease comprises the meganuclease, the zinc finger nuclease (ZFN), the TALE nuclease (TALEN), or the restriction endonuclease (RE).
33. The vector system of any one of paragraphs 17-32, further comprising a vector encoding a homologous recombination enhancer protein.
34. An RNA molecule encoded by the engineered nucleic acid construct of any one of paragraphs 1-16.
35. An engineered nucleic acid-enzyme construct comprising:
   a) a non-coding RNA (ncRNA) comprising:
      i) an msr locus encoding the msr RNA portion of a multi-copy single-stranded DNA (msDNA); and
      ii) an msd locus encoding the msd RNA portion of the msDNA;
   b) a heterologous nucleic acid inserted at or within a location selected from: the msd locus, upstream of the msr locus, upstream of the msd locus, and downstream of the msd locus; and
   c) a sequence encoding a reverse transcriptase (RT), or a domain thereof comprising:
      i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or
      ii) a polypeptide listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table C; and
   wherein, the RT optionally does not comprise a polypeptide listed in Table X.
36. An engineered nucleic acid-enzyme construct comprising:
   a) a non-coding RNA (ncRNA) comprising:
      i) an msr locus encoding the msr RNA portion of a multi-copy single-stranded DNA (msDNA); and
      ii) an msd locus encoding the msd RNA portion of the msDNA;
   wherein the ncRNA comprises:
      i) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B; and/or
      ii) an ncRNA having a conserved structure of any one of the ncRNA structures of FIGS. 2-27; and
      wherein the ncRNA optionally excludes any ncRNA associated in nature with any one of the retron reverse transcriptases of Table X;
   b) a heterologous nucleic acid inserted at or within a location selected from: the msd locus; upstream of the msr locus; upstream of the msd locus; and downstream of the msd locus; and
   c) a reverse transcriptase (RT), or a portion thereof, wherein the RT is capable of synthesizing a DNA copy of at least a portion of the msd locus encoding the msDNA.
37. An engineered nucleic acid-enzyme construct comprising:
   a) a non-coding RNA (ncRNA) comprising:
      i) an msr locus encoding the msr RNA portion of a multi-copy single-stranded DNA (msDNA); and
      ii) an msd locus encoding the msd RNA portion of the msDNA;
   wherein, the ncRNA comprises:
      i) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B; and/or
      ii) an ncRNA having a conserved structure of any one of the ncRNA structures of FIGS. 2-27; and
      wherein the ncRNA optionally excludes any ncRNA associated in nature with any one of the retron reverse transcriptases of Table X;
   b) a heterologous nucleic acid inserted at or within a location selected from: the msd locus, upstream of the msr locus, upstream of the msd locus, and downstream of the msd locus; and
   c) a reverse transcriptase (RT) or a domain thereof:
   wherein the RT comprises:
   i) an RT listed in Table A, or an RT having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an RT listed in Table A; and/or
   ii) a consensus sequence listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an amino acid sequence listed in Table C; and
   wherein the RT does not optionally comprise an RT listed in Table X.
38. An isolated host cell comprising the engineered nucleic acid construct of any one of paragraphs 1-16, the vector system of any of paragraphs 17-33, the RNA molecule of paragraph 34, or the engineered nucleic acid-enzyme construct of any one of paragraphs 35-37.
39. The isolated host cell of paragraph 38, wherein the host cell is a prokaryotic, archeon, or eukaryotic host cell.
40. The isolated host cell of paragraph 38, wherein the eukaryotic host cell is a mammalian host cell.
41. The isolated host cell of paragraph 39, wherein the eukaryotic host cell is a non-human host cell.
42. The isolated host cell of paragraph 40, wherein the mammalian host cell is a human host cell.
43. The isolated host cell of any one of paragraphs 38-42, wherein the host cell is an artificial cell or genetically modified cell.
44. A pharmaceutical composition comprising:
   a) the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, the engineered nucleic acid-enzyme construct of any one of paragraphs 35-37, and/or the isolated host cell of any one of paragraphs 38-43; and
   b) a pharmaceutically acceptable carrier.
45. A pharmaceutical composition comprising:
   a) a lipid nanoparticle (LNP); and
   b) the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, and/or the engineered nucleic acid-enzyme construct of any one of paragraphs 35-37.
46. The pharmaceutical composition of paragraph 45, wherein the LNP encapsulates the engineered nucleic acid construct, ncRNA, vector system, RNA molecule, and/or engineered nucleic acid-enzyme construct.
47. The pharmaceutical composition of paragraph 45 or 46, wherein the lipid nanoparticle comprises:
   a) one or more ionizable lipids;
   b) one or more structural lipids;
   c) one or more PEGylated lipids; and
   d) one or more phospholipids.
48. The pharmaceutical composition of paragraph 47, wherein the one or more ionizable lipids is selected from the group consisting of those disclosed in Table 2.
49. The pharmaceutical composition of paragraph 47 or 48, wherein the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.
50. The pharmaceutical composition of any one of paragraphs 47-49, wherein the one or more PEGylated lipids are selected from the group consisting of PEG-c-DOMG, PEG-DMG, PEG-DLPE, PEG-DMPE, PEG-DPPC, and PEG-DSPE.
51. The pharmaceutical composition of any one of paragraphs 47-50, wherein the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.
52. The pharmaceutical composition of any one of paragraphs 47-51, wherein the lipid nanoparticle comprises about 48.5 mol % ionizable lipid, about 10 mol % phospholipid, about 40 mol % structural lipid, and about 1.5 mol % of PEG lipid.
53. The pharmaceutical composition of any one of paragraphs 47-52, wherein the lipid nanoparticle comprises about 48.5 mol % ionizable lipid, about 10 mol % phospholipid, about 39 mol % structural lipid, and about 2.5 mol % of PEG lipid.
54. The pharmaceutical composition of any one of paragraphs 47-53, wherein the LNP further comprises a targeting moiety operably connected to the LNP.
55. The pharmaceutical composition of any one of paragraphs 47-54, wherein the LNP further comprises one or more additional components selected from the group consisting of DDAB, EPC, 14PA, 18BMP, DODAP, DOTAP, and C12-200.
56. The pharmaceutical composition of paragraph 45, wherein the lipid nanoparticle comprises at least one cationic lipid selected from the group consisting of: a lipid in Table 2, a lipid having a structure of Formula (I), a lipid having a structure of Formula (II), a lipid having a structure of Formula (III), a lipid having a structure of Formula (IV), a lipid having a structure of Formula (V), a lipid having a structure of Formula (VI), and combinations thereof.
57. A kit comprising the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, the engineered nucleic acid-enzyme construct of any one of paragraphs 35-37, the host cell of any one of paragraphs 38-43, or the pharmaceutical composition of any one of paragraphs 44-56, and instructions for genetically modifying a cell with said engineered nucleic acid construct, ncRNA, vector system, host cell, or pharmaceutical composition.
58. A method of modifying a target DNA sequence in a host (e.g., mammalian) cell, the method comprising introducing into the mammalian cell the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, the engineered nucleic acid-enzyme construct of any one of paragraphs 35-37, or the pharmaceutical composition of any one of paragraphs 44-56, to allow production of the msDNA in the host (e.g., mammalian) cell, wherein the heterologous nucleic acid in the msDNA is integrated into the genome of the host (e.g., mammalian) cell at the target DNA sequence by homology-dependent recombination.
59. The method of paragraph 58, wherein the modifying comprises introducing an insertion, deletion and/or substitution into the target DNA sequence.
60. A method of treating a disease or condition in a subject in need thereof, the method comprising administering a therapeutically effective amount of the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, the engineered nucleic acid-enzyme construct of any one of paragraphs 35-37, the host cell of any one of paragraphs 38-43, or the pharmaceutical composition of any one of paragraphs 44-56 to the subject, thereby treating the disease or condition in the subject.
61. A method of treating a disease or condition in a subject in need thereof, the method comprising administering a therapeutically effective amount of the host cell of any one of paragraphs 38-43 to the subject, thereby treating the disease or condition in the subject.
62. The method of paragraph 61, wherein the host cell is autologous to the subject.
63. The method of paragraph 61, wherein the host cell is allogeneic to the subject.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1A is a schematic that depicts naturally occurring retrons from genomic DNA stage through production of the msDNA chimeric microsatellite molecule. Retrons are encoded in the bacterial genome and comprise a non-coding RNA (ncRNA) portion and a portion encoding a specialized reverse transcriptase (RT). The ncRNA and the RT initially are transcribed from the retron DNA as a single polycistronic message. The initial transcript is processed resulting in the removal or separation of the transcript encoding the retron RT. The remaining transcript is the ncRNA, which undergoes folding to form a secondary structure having several characteristic stem-loops and a duplex formed between the 5′ and 3′ regions of the ncRNA (i.e., the a1/a2 duplex). The folded ncRNA is recognized by the accompanying RT which is separately translated and provided in trans. The translated RT typically recognizes certain secondary structures in the ncRNA, and binds the RNA template downstream from the msd region. The RT initiates reverse transcription of the RNA towards its 5′ end, starting from the 2′-end of a conserved guanosine (G) residue found immediately after a double-stranded RNA structure (the a1/a2 region) within the ncRNA. A portion (i.e., the msd region) of the ncRNA serves as a template for reverse transcription, and reverse transcription terminates before reaching the msr locus. During reverse transcription, cellular RNase H degrades the segment of the ncRNA that serves as template, but not other parts of the ncRNA. The result of the reverse transcription, the msDNA (lower right of schematic), remains covalently attached to the RNA template via the 2′-5′ phosphodiester bond, and base-pairs with the RNA template using the 3′ end of the msDNA.

FIG. 1B is a schematic depicting an embodiment of a recombinant retron contemplated by this disclosure. In this embodiment, a nucleic acid molecule comprises a nucleotide sequence encoding a retron ncRNA region (the msr/msd region) is depicted at the top left. The msr region has been modified by introducing one or more nucleotide modifications (e.g., a nucleotide substitution, deletion, or insertion). For example, it may be desirable to introduce one or more nucleotide substitutions in the msr to enhance functionality (e.g., binding of the corresponding ncRNA to RT, improved stability, improved folding, etc.). The modified msr is referred to as msr′. In addition, the msd has been modified by introducing a heterologous nucleotide sequence encoding an HDR donor template. Lastly, the retron DNA has been modified to introduce a nucleotide sequence encoding a guide RNA at the 3′ end of the retron DNA sequence. is configured on a DNA vector (e.g., a plasmid). The DNA is shown to be transcribed as a polycistronic message that includes the msr′/msd′ region (forming the ncRNA) which is fused at its 3′ end to the guide RNA (in other embodiments, the guide RNA could be fused to the 5′ end of the retron ncRNA. This intermediate is shown to form a complex with a reverse transcriptase provided in trans (e.g., by way of a separate expression vector or delivered mRNA). The top right schematic shows the formation of a complex between the recombinant ncRNA and the RT and the beginning of reverse transcription from the covalently-linked conserved guanosine (G) (i.e., the “priming G” or “priming guanosine”) using the msd RNA as a template sequence. Following the completion of reverse transcription and RNaseH degradation of the template RNA sequence, a recombinant msDNA is formed which comprises three modifications, as shown: (a) a guide RNA linked to the 3′ end of the msDNA, (b) a nucleotide change in the msr′, and (c) the reverse-transcribed single-strand DNA comprises a region that is an HDR donor template. Such a recombinant msDNA could then facilitate various genome modification applications in the cell, including genome editing with an RNA-guided nuclease provided to the cell in trans.

FIG. 1C is a schematic depicting a recombinant retron-based genome editing system described herein. In the case of genome editing involving an RNA-guided nuclease, the components of such a system may include (a) a guide RNA provided in cis (e.g., fused to the recombinant retron msDNA) and/or in trans (e.g., separately expressed in a cell), (b) a recombinant ncRNA (including at least a sequence encoding an HDR donor template and optionally a guide RNA fused to the ncRNA), (c) a reverse transcriptase, and (d) a programmable nuclease. These components are provided to a cell in the form of DNA and/or RNA and/or protein by a delivery means (e.g., LNPs, liposomes, virus-based delivery, or passive/active transport). Once inside the cell, the recombinant msDNA is formed. The msDNA and the programmable nuclease translocate to the nuclease to conduct gene editing at a target DNA site, thereby producing an edited DNA target.

FIG. 1D provides a simplified schematic of a the natural lifecycle of a retron. Retrons typically comprise a reverse transcriptase (RT) and two non-coding contiguous inverted sequences (msr and msd) transcribed as a single RNA that is folded into a specific secondary structure. The conserved NAXXH motif and VTG triplet in retron RTs are indicated. The RT binds downstream from the msd locus in the RNA, initiating reverse transcription of the RNA template towards its 5′ end, assisted by the 2′OH group present in a conserved branching G residue acting as a primer. Reverse transcription halts before the msr region is reached, and the resulting msDNA remains covalently attached to the RNA template via a 2′-5′ phosphodiester bond and base-pairing of the 3′ ends of the molecules.

FIG. 1E provides a detailed representation of the natural biological pathway of a retron in a cell, concluding with the generation of the msDNA satellite molecule. This figure parallels FIG. 1D but more completely depicts the stages of msDNA production. (1) depicts the retron locus which includes an ncRNA locus having an msr locus and a msd locus (both of which are non-coding) and a reverse transcriptase (RT) locus. The ncDNA locus and the RT locus are transcribed as a single RNA transcript, which is depicted in (2). The colors representing each component in (1) are carried through each of stages (2) through (6). Stages (3) and (4) depict the folding of the ncRNA portion into a series of stem loops, wherein the 5′ end and the 3′ ends of the ncRNA form a duplex. In addition, the position of the conserved branching guanosine residue having a 2′OH group is show. The branching guanosine serves as a future priming site for the reverse transcriptase. Stage (4) further shows that the region of the transcript encoding the reverse transcriptase is removed, separately being translated to produce the reverse transcriptase enzyme. In stage (5), the reverse transcriptase associates with the folded ncRNA and begins polymerization of a single strand of DNA (i.e., the reverse transcription product) from the primer site (i.e., the conserved branching guanosine residue having the 2′OH end) and using the msd RNA sequence as a template. Reverse transcription terminates at the msr region. The msd RNA template is exonucleotically removed, thereby resulting in a chimeric molecule comprising the msr RNA region which is covalently joined to the ssDNA transcription product through covalent linkage to the conserved guanosine primer residue. There is also a short duplex region that forms between the 3′ end of the msr RNA and the 3′ of the ssDNA reverse transcript product. The complete molecule is referred to as the “msDNA.”

FIG. 1F is a schematic depicting that the herein disclosed recombinant retron-based genome modification systems may be implemented as (a) cell recorder systems, (b) genome editing systems, and (c) recombineering systems. These uses are not intended to be limiting.

FIG. 1G is a schematic depicting various configurations contemplated for the recombinant retron disclosed herein. (1) shows the operonic structure of a wild type retron; (2) shows the operonic structure of a recombinant retron configured to encode an HDR donor template in the final msDNA molecule; (3) shows (2) but further modified to encode a guide RNA at the 3′ end of the retron; (4) shows (2) but further modified to encode a guide RNA in trans.

FIG. 1H is a schematic that emphasizes that any suitable configuration for presenting the components of the recombinant retron-based genome modification systems disclosed herein to a cell are contemplated, including where the RT and/or the programmable nuclease are provided in trans relative to the retron ncRNA. In some embodiments, the RT and the programmable nuclease may be provided as a fusion protein.

FIG. 1I depicts that the RT and programmable nuclease may be provided as fusion proteins (top, middle) or provided separate from one another.

FIG. 1J depicts that nuclear localization signals may be engineered into the polypeptides of the disclosure (e.g., a RNA-guide nuclease) to facilitate translocation into the nuclease of cell where editing occurs.

FIG. 1K is a schematic (not to scale) representation depicting an embodiment of genome editing, in which double-stranded break (DSB) created by a suitable nuclease (such as a CRISPR/Cas effector enzyme, a ZFN, TALEN, meganuclease, TnpB, IscB, or restriction enzymes (Res)) promotes the insertion of a donor or template sequence (shown here as a “marker” flanked by homologous sequences matching those flanking the DSB, provided on a “donor vector”).

FIG. 1L depicts the capability of three different kinds of retrons (Eco1-R1, Eco3-R2, Eco5-R3) to insert a 16 base pair insertion at a specific genomic site, the EMX1 gene.

FIG. 1M outlines a procedure used to evaluate the capability of a retron to produce genomic insertions.

FIG. 2 (SEQ ID NO:19970) is a schematic representation of a consensus secondary structure of a retron ncRNA nsr/nsd of Type IA/IIA1 retron produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 3 (SEQ ID NO:19971) is a schematic representation of a consensus secondary structure of a retron ncRNA msr/msd of Type IB1 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 4 (SEQ ID NO:19972) is a schematic representation of a consensus secondary structure of a retron ncRNA msr/msd of Type IB2 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 5 (SEQ ID NO:19973) is a schematic representation of a consensus secondary structure of a retron ncRNA msr/msd of Type IC retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 6 (SEQ ID NO:19974) is a schematic representation of a consensus secondary structure of a retron ncRNA msr/msd of Type IIA other retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 7 (SEQ ID NO:19975) is a schematic representation of a consensus secondary structure of a retron ncRNA msr/msd of Type IIA2 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 8 (SEQ ID NO:19976) is a schematic representation of a consensus secondary structure of a retron ncRNA msr/msd of Type IIA3 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 9 (SEQ ID NO:19977) is a schematic representation of a consensus secondary structure of a retron ncRNA msr/msd of Type IIA4 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 10 (SEQ ID NO:19978) is a schematic representation of a consensus secondary structure of a retron ncRNA msr/msd of Type IIA5 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 11 (SEQ ID NO:19979) is a schematic representation of a consensus secondary structure of a retron ncRNA msr/msd of Type IIIA1 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 12 (SEQ ID NO:19980) is a schematic representation of a consensus secondary structure of a retron ncRNA msr/msd of Type IIIA2 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 13 (SEQ ID NO:19981) is a schematic representation of a consensus secondary structure of a retron ncRNA msr/msd of IIIA3 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 14 (SEQ ID NO:19982) is a schematic representation of a consensus secondary structure of a retron ncRNA msr/msd of Type IIIA4 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 15 (SEQ ID NO:19983) is a schematic representation of a consensus secondary structure of a retron ncRNA msr/msd of Type IIIA5 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 16 (SEQ ID NO:19984) is a schematic representation of a consensus secondary structure of a retron ncRNA msr/msd of Type IIIunk retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 17 (SEQ ID NO:19985) is a schematic representation of a consensus secondary structure of a retron ncRNA msr/msd of Type IV retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 18 (SEQ ID NO:19986) is a schematic representation of a consensus secondary structure of a retron ncRNA msr/msd of Type IX retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 19 (SEQ ID NO:19987) is a schematic representation of a consensus secondary structure of a retron ncRNA msr/msd of Type V retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 20 (SEQ ID NO:19988) is a schematic representation of a consensus secondary structure of a retron ncRNA msr/msd of Type VI retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 21 (SEQ ID NO:19989) is a schematic representation of a consensus secondary structure of a retron ncRNA msr/msd of Type XI Group 1 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 22 (SEQ ID NO:19990) is a schematic representation of a consensus secondary structure of a retron ncRNA msr/msd of Type XI Group 2 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 23 (SEQ ID NO:19991) is a schematic representation of a consensus secondary structure of a retron ncRNA msr/msd of Type XII retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 24 (SEQ ID NO:19992) is a schematic representation of a consensus secondary structure of a retron ncRNA msr/msd of Type XIII retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 25 (SEQ ID NO:19993) is a schematic representation of a consensus secondary structure of a retron ncRNA msr/msd of Type XIV retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 26 (SEQ ID NO:19994) is a schematic representation of a consensus secondary structure of a retron ncRNA msr/msd of Ec107 retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 27 (SEQ ID NO:19995) is a schematic representation of a consensus secondary structure of a retron ncRNA msr/msd of Outgroup A retrons produced by computational structural alignment of ncRNA sequences from Table B as described in Example 3.

The colored dots represents the probability a base is at that location (e.g., red circle represents the presence of a base in 97% of the cases, black represents the presence of a base in 90-97% of the cases, grey represents the presence of a base in 75-90% of the cases, and white represents the presence of a base in 50-75% of the cases), as opposed to a gap (no base), whereas the colored letters represent bases that are conserved to different degrees (e.g., with red representing 97%+ conserved, black being 90%+ conserved, and grey at least 75% conserved). Each highlighted base-pair represents a significantly covarying basepair.

FIG. 28 is a phylogenetic tree of RT sequences constructed in accordance with Example 3.

FIG. 29 is a structural representation of the retron loci associated with each retron type in FIG. 28.

FIG. 30 shows the position of certain retrons (EcoI, Eco3, Eco5, AcoI, RTX003_2042, RTX003_6083v1, and RTX003_6943) within the phylogenetic retron tree of FIG. 28.

FIG. 31A is a plasmid map of an exemplary retron (EcoI) tested in the Examples herein.

FIG. 31B is a linear representation in 5′ to 3′ direction of the plasmid map of FIG. 31A.

FIG. 31C is a plasmid map of an exemplary retron (RTX3_6083v1) tested in the Examples herein.

FIG. 31D is a linear representation in 5′ to 3′ direction of the plasmid map of FIG. 31C.

FIG. 32 is a representation of a plasmid-based assay used to measure retron precise edits and indels as performed in the Examples. In Step 1, a plasmid (e.g., that of FIG. 31A or FIG. 31C) is transfected into human cells (e.g., HEK293t cells) which are engineered to express Cas9. Editing is allowed to occur for 72 hours at 37° C. In Step 2, the genomic DNA is extracted from the cells and used to prepare a next-generation sequencing (NGS) library for sequencing. The library is sequenced over the target site (e.g., EMX1) of editing to generate sequence reads. In Step 3, the sequencing reads are analyzed to obtain a frequency of sequence reads containing the desired edit (percentage of precision editing) and a frequence of indels at the desired edit site (percentage of indels).

FIG. 33 is an equivalent representation of FIG. 32.

FIG. 34 is a representation of a methodology for transfecting HEK293T cells. Cells were seeded in 24 well plate one day prior to transfection. Appropriate amount of plasmid and transfection reagent (e.g., Lipofectamine 3000) were mixed and transferred to cells. After 72 hours incubation, genomic DNA was extracted and the target edit region was amplified into sequencing libraries. Sequencing data was analyzed by CRISPResso2 and percentage of precise edit and indels were calculated.

FIG. 35 (SEQ ID NOs:19996-20003) (Top to Bottom) is an example of reference sequence and desired editing outcome for Eco3 retron at the EMX1 genomic site. Analysis of editing outcomes is performed using CRISPresso2 pipeline. In this example, the editing template inserts a 10 bp insertion into the EMX1 gene (TTACGTCTGC) (SEQ ID NO: 19931) along with a 6 bp substitution to mutate the PAM sequence (GAAGGG>AAAGTT) (SEQ ID NO: 19954).

FIG. 36 shows the results of plasmid-based assay (e.g., according to FIG. 33) demonstrating up to about 0.3% precise edits and as low as 40% indels with Eco1 retron in Cas9 expressing HEK293T cells. The plasmid that encodes Eco1 RT and Eco1 ncRNA-sgRNA fusion targeting EMX1 was transfected via lipofection using two different amounts of Lipofectamine.

FIG. 37 shows the results of plasmid-based assay (e.g., according to FIG. 33) demonstrating up to about 0.1% precise edits and as low as 3% indels with AcoI. Acol retron has not been experimentally validated to produce msDNA. Precise editing activity observed in this experiment strongly support that Aco1 retron is capable of generating msDNA inside human cells.

FIG. 38 shows the results of plasmid-based assay (e.g., according to FIG. 33) demonstrating up to about 0.3% precise edits and as low as 5% indels with RTX003_2042. This retron can achieve a comparable precise editing to Eco1 but with significantly lower indels (10-fold). RTX003_2042 is a novel retron and precise editing activity observed in this experiment strongly support that RTX003_2042 retron could generate msDNA inside human cells.

FIG. 39A shows the results of plasmid-based assay demonstrating up to about 0.05˜0.08% precise edits and as low as 2.5˜4% indels with RTX003_6083v1 and 6943. Both are novel retrons and precise editing activity observed in this experiment strongly support that RTX003_6083v1 and 6943 retron could generate msDNA inside human cells.

FIG. 39B shows follow up experiments using the same assay of FIG. 39A indicated that RTX3_6083v1 and RTX3_6943 generated 3-4 fold more precise edits than Eco1 while indels generated from these two retrons were 2-3 fold lower. RTX3_2042 showed precise editing at similar frequencies to Eco1 but had more variability than other samples.

FIG. 39C shows follow up experiments using the same assay of FIG. 39A indicated that RTX3_6083v1 and RTX3_6943 generated 3-4 fold more precise edits than Eco1 while indels generated from these two retrons were 2-3 fold lower. RTX3_2042 showed precise editing at similar frequencies to Eco1 but had more variability than other samples.

FIG. 39D shows the results of plasmid-based assay demonstrating up to about 0.7% precise edits and as low as ˜4% indels with RTX003_0637, RTX003_1262, and RTX003_6342 compared to empty vector and EcoI. RTX003_0637, RTX003_1262, and RTX003_6342 are novel retrons and precise editing activity observed in this experiment strongly support that these retrons could generate msDNA inside cells.

FIG. 39E shows the results of plasmid-based assay demonstrating precise editing and indel generation for an array of retrons, including EcoI, Eco3, RTX3_2042_RT inactivated, RTX3_2042, RTX3_6083v1, RTX3_6943, RTX3_6943, RTX3_1262, RTX3_6342S, and RTX3_6342L. up to about 2.5% precise edits and as low as ˜4% indels with EcoI, Eco3, RTX3_2042_RT_inactivated, RTX3_2042, RTX3_6083v1, RTX3_6943, RTX3_6943, RTX3_1262, RTX3_6342S, and RTX3_6342L compared to empty vector and EcoI as controls.

FIG. 40 is a representation of an two-RNA editing assay used in the Examples to measure the relative editing efficiency of exemplary retrons using electroporation-based delivery of two RNA components into HEK293T cells. Appropriate amount of RT mRNA and ncRNA-sgRNA fusion were mixed and electroporated to cells. After 72 hours incubation, genomic DNA was extracted and the targeting region was amplified into sequencing libraries. Sequencing data was analyzed by CRISPResso2 and precise edit and indels were calculated.

FIG. 41 shows the results of two-RNA system (RT mRNA+ncRNA-sgRNA fusion) delivered to Cas9 expressing HEK293T cells by electroporation. Eco1, Eco3 and Eco5 retrons were tested. Results showed precise edits (left graph) up to 0.4% for Eco3 and as low as 10% indels (right graph) for Eco3. Precise edits mediated by Eco3 increased with augmenting amount of ncRNA-sgRNA fusion from 1:2 to 1:4 ratio between RT mRNA and ncRNA-sgRNA fusion.

FIG. 42 shows the results of titration of two-component Eco3 RNA system (RT mRNA+ncRNA-sgRNA fusion) delivered to Cas9 expressing 293T cells by electroporation. The RT mRNA and the ncRNA were mixed at ratios of 1:2, 1:3, 1:4, 1:5, 1:8, 1:10, respectively, and delivered at two different amounts of RT mRNA (0.2 or 0.5 g). On left, data showed that Eco3 at 0.5 μg produced highest percentage of precise edits at a 1:3 and 1:5 ratio of RT mRNA to ncRNA. On right, data further showed that a more equivalent ratio of RT mRNA to ncRNA resulted in a trend of lower percentage of indels.

FIG. 43 is a representation of a three-RNA retron editing system which involves delivery by electroporation of three RNA components (RT mRNA, retron ncRNA-sgRNA fusion, and Cas9 mRNA) into HEK293T cells. Appropriate amount of RT mRNA, ncRNA-sgRNA fusion, and Cas9 mRNA were mixed and electroporated to cells. After 72 hours incubation, genomic DNA was extracted and targeting region was amplified into sequencing libraries. Sequencing data was analyzed by CRISPResso2 and precise edit and indels were calculated.

FIG. 44 shows the results of Cas9 mRNA titration of three-component Eco3 RNA system (RT mRNA+ncRNA-sgRNA fusion+Cas9 mRNA) delivered to 293T cells by electroporation. The RT mRNA and the ncRNA-sgRNA fusion were mixed at given amounts on the graph and the amount Cas9 mRNA was titrated. At 0.2 μg of Cas9 mRNA, up to 0.1% of precise editing was observed. While the editing efficiency is an order of magnitude lower than two RNA system, the editing occurred by specific action of Cas9 and retron, since absence of either abrogated the editing.

FIG. 45 depicts a process of lipofection using three RNA system in HEK293T cells. Cells were seeded in 96 well plate one day prior to transfection. Appropriate amount of RT mRNA, ncRNA-sgRNA fusion, Cas9 mRNA and Lipofectamine reagent were mixed and transferred to cells. After 72 hours incubation, genomic DNA was extracted and targeting region was amplified into sequencing libraries. Sequencing data was analyzed by CRISPResso2 and precise edit and indels were calculated.

FIG. 46 shows the results of three-component Aco1 RNA system (RT mRNA+ncRNA+Cas9 mRNA) delivered to HEK293T cells by lipofection. The RT mRNA, the ncRNA and the Cas9 mRNA were mixed at amounts indicated in the graph and transfected to HEK293T cells. 56 bp insertion and 6 bp deletion at EMX1 locus was scored as precise edits and ˜0.1% of cell population has undergone precise editing on the left graph. The editing was dependent on Cas9 nuclease since its absence abrogated the editing. The frequency of indels was about 1.5% on the right graph.

FIG. 47 shows the results of minimal Cas9 nuclease activity when sgRNA is fused to ncRNA of Eco3 retron. Cas9 activity was evaluated by frequency of indels. 1 g of ncRNA-sgRNA fusion shows 20-fold lower activity than equimolar separated sgRNA alone. In parallel, activity of chemical modified vs unmodified sgRNA was compared and the former shows 6-fold higher activity than the latter at the condition described in the graph.

FIG. 48 is a representation of the all-RNA editing assay used in the Examples to measure the relative editing efficiency of the sample retrons in an all-RNA format, modified with a step of in trans guide RNA spike-in. Electroporation using three RNA system+sgRNA trans spike-in in HEK293T cells. Appropriate amount of RT mRNA, ncRNA-sgRNA fusion, Cas9 mRNA, and sgRNA were mixed and electroporated to cells. After 72 hours incubation, genomic DNA was extracted and targeting region was amplified into sequencing libraries. Sequencing data was analyzed by CRISPResso2 and precise edit and indels were calculated.

FIG. 49 shows the results of guide RNA spike-in in all RNA system (RT mRNA+ncRNA-sgRNA fusion+Cas9 mRNA+sgRNA) delivered to HEK293T cells by electroporation. At given amount of Cas9 and RT mRNA on the graph, the amount of guide RNA spike-in is titrated at 50, 100 and 200 ng. The titration was done at two different ratios of RT mRNA: ncRNA-sgRNA fusion=1:6 or 1:8. The guide RNA spike-in in all RNA system increased precise editing up to ˜50 fold. The increasing amount of guide RNA gradually increased precise editing and 1:8 of RT mRNA:ncRNA-sgRNA fusion performed slightly better than 1:6, reaching 13% of precise editing. On the right graph, frequency of indels is shown for respective conditions.

FIG. 50 represents a lipofection process using three RNA system+gRNA trans spike-in in HEK293T cells. Cells were seeded in 96 well plate one day prior to transfection. Appropriate amount of RT mRNA, ncRNA-sgRNA fusion, Cas9 mRNA, sgRNA and Lipofectamine reagent were mixed and transferred to cells. After 72 hours incubation, genomic DNA was extracted and targeting region was amplified into sequencing libraries. Sequencing data was analyzed by CRISPResso2 and precise edit and indels were calculated.

FIG. 51 shows the results of guide RNA spike-in (i.e., delivering a separate molecule bolus of guide RNA which in all RNA system (RT mRNA+ncRNA-sgRNA fusion+Cas9 mRNA+sgRNA) delivered to HEK293T cells by lipofection. At given amount of RT mRNA, ncRNA-sgRNA fusion, and Cas9 mRNA on the graph, the amount of guide RNA spike-in is titrated at 2, 5 and 10 ng. The guide RNA spike-in in all RNA system increased precise editing up to 3.5 fold, 12% of efficiency. The increasing amount of guide RNA at this range did not further increase precise editing. The precise editing is completely dependent on the presence of Retron machinery. On the right graph, frequency of indels is shown for respective conditions.

FIG. 52 shows the results of ncRNA-sgRNA fusion separation in all RNA system (RT mRNA+Cas9 mRNA+either ncRNA-sgRNA fusion OR separate ncRNA+sgRNA) delivered to HEK293T cells by lipofection. At given amount of RT mRNA and Cas9 mRNA on the graph, the amount of guide RNA spike-in is titrated at 0, 2, 5, 10, 50 and 100 ng. At 10 ng guide RNA, precise editing peaked at 2.23% compared to 1.78% with ncRNA-sgRNA fusion. The increasing amount of guide RNA at this range did not further increase precise editing. On the right graph, frequency of indels is shown for respective conditions.

FIG. 53 is a schematic of improved templates used for in vitro transcription to produce ncRNA (left or A) and ncRNA modifications (right or B). (A) relates to the optimization of RNA production by in vitro transcription. Previously made in vitro transcription experiments to produce RNA used a double-stranded DNA template containing a 3′ overhang (on same strand as T7 promoter sequence). A new template with a blunt end was designed and tested and as shown in FIG. 54 results in increased precise editing efficiency. (B) relates to modified ncRNAs which are modified by addition of an MS2 stem loop hairpin at the 3′ end of the ncRNA. Without being bound by theory, the MS2 loop helps stabilize the ncRNA and results in significantly improved precise editing efficiency, as shown in FIG. 54.

FIG. 54 shows the results of ncRNA-sgRNA fusion separation in 4 component all-RNA system (RT mRNA+Cas9 mRNA+ncRNA+sgRNA) delivered to HEK293T cells by Lipofectamine MessengerMAX. All RNA was transfected at a fixed amount as shown on the graphs. Using RNA generated from linearized plasmid template containing a 3′ overhang produced 1.35% precise edits. Using RNA generated from the improved linearized plasmid template containing a blunt end increased precise editing to 5.94%. Adding an MS2 stem loop to the 3′ end of the ncRNA (blunt end) further increased precise editing to 12.39%. On the right graph, frequency of indels is shown for respective conditions.

FIG. 55 (SEQ ID NO: 19969) provides schematics for end protection of RNA from cellular nuclease activity by capping and tailing. In (A), a 7-methylguanosine cap0 was added to 5′ triphosphate of RNA. In (B), a poly-A tail was added to the 3′ end by enzymatic addition. Tail length is estimated over 50 nucleotides. In (C), RNA containing both a 5′ cap and a 3′ tail is shown. Results are shown in FIG. 56.

FIG. 56 shows the result of end protection of ncRNA-sgRNA fusion by cap and tail in 4 component all-RNA system (RT mRNA+Cas9 mRNA+ncRNA+sgRNA) delivered to HEK293T cells by Lipofectamine MessengerMAX. All RNA was transfected at a fixed amount RT mRNA 100 ng, ncRNA-sgRNA 400 ng, Cas9 mRNA 100 ng, and sgRNA 5 ng. ncRNA-gRNA fusion was either capped (+cap −tail) or poly-A tailed (−cap +tail) or both capped and poly-A tailed (+cap +tail). Using RNA without end protection (−cap −tail) produced ˜4.5% precise edits and the editing was dependent on retron since the absence of RT abrogated precise editing. Using RNA with either or both protection by cap and tail produced lower precise editing (left graph) but lowered indels (right graph) than without cap and tail.

FIG. 57 shows the result that shortening msd stems can modulate precising editing in a retron specific manner. Modified retrons include RTX3_4536 (long (L) and short (S) versions), RTX3_6279 (long (L) and short (S) versions), RTX3_6342 (long (L) and short (S) versions), RTX3_6438 (long (L) and short (S) versions), RTX3_6549 (long (L) and short (S) versions), and RTX3_6605 (long (L) and short (S) versions).

FIG. 58 shows the result that shortening msd stems can modulate precising editing in a retron specific manner. Modified retrons include RTX3_5752 (long (L) and short (S) versions), RTX3_6221 (long (L) and short (S) versions), and RTX3_6034 (long (L) and short (S) versions).

FIG. 59A Demonstrates that four retrons from various clades are capable of insertions of up to 100 bps to EMX1 locus. Figure shows the results of testing different template lengths for four different retrons in all RNA system delivered to HEK293T cells by lipofection. At given amount of RT mRNA and Cas9 mRNA, ncRNA-sgRNA fusion with different template lengths ranging from 10 to 100 bp were added. For 100 bp insertions, precise editing was 1.04% for Eco3, 1.52% for Aco1, 1.37% for R2042, and 0.05% for R6943. On the bottom row of graphs, frequency of indels is shown for respective conditions.

FIG. 59B Demonstrates that four retrons from various clades are capable of insertions of up to 100 bps to EMX1 locus. Figure shows indel results corresponding with the constructs of FIG. 59A, graphing frequency of indels for respective conditions.

FIG. 60A Demonstrates that additional sgRNA targeting the same genomic locus increases the indel and insert frequencies. This figure reports same conditions as FIGS. 59A-59B but with sgRNA added, which increases the frequency of precise editing and indels. For 100 bp insertions, precise editing was 1.82% for Eco3, 4.50% for Aco1, 4.40% for R2042 and 0.38% for R6943. On the bottom row of graphs, frequency of indels is shown for respective conditions.

FIG. 60B Demonstrates that additional sgRNA targeting the same genomic locus increases the indel and insert frequencies. This figure reports the frequency of indels associated with the retrons of FIG. 60A.

FIG. 61A is a schematic showing unedited and edited allele by GFP gene insertion at EMX1 locus using retron editing system. Primer pair EMX1 Forward and Reverse hybridizes just outside of 5′ and 3′ homology arms and amplifies 169 bp on the unedited allele. On the edited allele, they amplify 1433 bp spanning GFP gene insertion. Primers to detect 5′ and 3′ junctions are indicated on the edited allele. 5′ junction on the edited allele is amplified by primer pair: EMX1 Forward and 5′ Junction GFP Reverse and 3′ junction is amplified by primer pair: 3′ Junction GFP forward and EMX1 Reverse. Neither 5′ Junction GFP Reverse nor 3′ Junction GFP Forward primer binds to unedited allele.

FIG. 61B Demonstrates three retrons from various clades are capable of GFP integration at EMX1 locus. The figure provides qPCR data depicting GFP insertion at 5′ or 3′ junctions at EMX1 locus by three retrons. ncRNA-sgRNA engineered to contain GFP gene insert was transfected to HEK293T cells, together with RT mRNA of each retron, Cas9 mRNA, with/without additional sgRNA. Genomic DNA was analyzed by indicated primer pairs to detect GFP gene insertion at 5′ or 3′ junctions on the edited allele. ΔCt was obtained by subtracting +RT sample's Ct value from −RT sample's Ct value for relative quantification of GFP insertion at EMX1 locus. The bigger value of ΔCt indicates the higher frequency of insert in the sample. With all three retrons, amplicons of both 5′ and 3′ junctions are detectable significantly above background level. Amplicon of 3′ junction is more abundant than that of 5′ junction probably because insertion at 3′ is facilitated by more enriched 5′ end of msDNA from reverse transcription.

FIG. 61C Demonstrates that two retrons are capable of GFP integration at EMX1 locus. The figure provides qPCR data depicting GFP insertion at 5′ or 3′ junctions at EMX1 locus by two retrons. ncRNA-sgRNA engineered to contain GFP gene insert was transfected to HEK293T cells, together with RT mRNA of each retron, Cas9 mRNA, with/without additional sgRNA. Genomic DNA was analyzed by indicated primer pairs in the table to detect GFP gene insertion at 5′ or 3′ junctions on the edited allele. ΔCt was obtained by subtracting junction Ct value from EMX1 Ct value for relative quantification of GFP insertion at EMX1 locus. With all two retrons, amplicons of both 5′ and 3′ junctions are detectable significantly above background level. Amplicon of 3′ junction is slightly more abundant than that of 5′ junction probably because insertion at 3′ is facilitated by more enriched 5′ end of msDNA from reverse transcription.

FIG. 61D shows PCR products for Aco1 were run on Agilent Tapestation High Sensitivity D1000 screentape. In the presence of RT (+RT sample, left on the gel), the expected size of amplicons were detected at both 5′ and 3′ junctions of edited alleles as indicated by arrows. Edited alleles were not detected in the absence of RT (−RT sample, right on the gel). Triangles indicate the expected amplicon for unedited alleles, which is present in both −RT and +RT samples.

FIG. 62A Demonstrates that order of sgRNA and ncRNA within fused RNA wherein sgRNA is positioned upstream of ncRNA is more active in precise editing and indels. Results of testing different ncRNA-sgRNA fusion orders for three different retrons in all RNA system delivered to HEK293T cells by lipofection. At given amount of RT mRNA and Cas9 mRNA, ncRNA fusions with different order were added. For Eco3, Aco1 and R2042, precise editing was increased was higher with sgRNA-ncRNA compared to ncRNA-sgRNA. On the bottom row of graphs, frequency of indels is shown for respective conditions.

FIG. 62B Demonstrates that where sgRNA is positioned downstream of ncRNA, additional sgRNA compensates for lower editing activity of configuration. Same as previous figure but with the addition of sgRNA, In the presence of additional of sgRNA, the differences between ncRNA-sgRNA and sgRNA-ncRNA depends on each retron. For Eco3 and R2042, sgRNA-ncRNA performed better than ncRNA-sgRNA. For Aco1, ncRNA-sgRNA performed slightly better than sgRNA-ncRNA. On the bottom row of graphs, frequency of indels is shown for respective conditions.

FIG. 63 Demonstrates that RT and Cas9 are required for precise editing. Results of testing negative controls in all RNA system delivered to HEK293T cells by lipofection. RT and Cas9 are required for precise editing. When any one of these components is removed, precise editing is undetectable or at background levels. On the bottom graphs, frequency of indels is shown for respective conditions.

FIG. 64 Demonstrates that separation of ncRNA and gRNA does not result in the decreased frequency of precise edits. Results of testing ncRNA-sgRNA fusions versus separate ncRNA for four different retrons in all RNA system delivered to HEK293T cells by lipofection. At given amount of RT mRNA, Cas9 mRNA and additional sgRNA described underneath of graph, ncRNA/ncRNA-sgRNA/sgRNA-ncRNA were added. All insertion templates tested were 25 bp. The best ncRNA format depends on each retron. For Eco3, the highest precise editing was observed with separate ncRNA+additional sgRNA (9.88%). For Aco1, the highest precise editing was observed with ncRNA-sgRNA+additional sgRNA (2.96%). For R2042, the highest precise editing was observed with separate ncRNA+additional sgRNA (7.4%). For R6943, the highest precise editing was observed with ncRNA-sgRNA fusion+additional sgRNA (0.39%). On the bottom row of graphs, frequency of indels is shown for respective conditions.

FIG. 65 Demonstrates that retrons can achieve precise editing at AAVS1 gene locus. Results of testing four different retrons to insert 25 bp template into AAVS1 locus. Retrons were delivered as all RNA to HEK293T cells by lipofection. For all four retrons, the best precise editing was observed in the presence of additional sgRNA and ranged from 0.73% to 2.05% depending on the retron used. On the right, frequency of indels is shown for respective conditions.

FIG. 66 Demonstrates that retrons can achieve precise editing at AAVS1 gene locus. Results of negative controls for AAVS1 experiments in previous figure. Removing either RT, ncRNA-sgRNA or Cas9 mRNA abrogates precise editing. On the bottom row of graphs, frequency of indels is shown for respective conditions.

FIG. 67 Demonstrates that template for either target or non-target strand can be used for precise editing. Results of testing templates on different strands relative to Cas9 sgRNA target strand for two different retrons in all RNA system delivered to HEK293T cells by lipofection. At given amount of RT mRNA, Cas9 mRNA and additional sgRNA, ncRNA-sgRNA was added. All insertion templates tested were 25 bp. All previous figures encoded the insertion template on the same strand targeted by the Cas9 sgRNA denoted by the letter T. For Eco3, in the presence of additional sgRNA, precise editing was 1.67% when template was on the target strand and 0.85% when the template was on the non-target strand. For Aco1, in the presence of additional sgRNA, precise editing was 2.96% when template was on the target strand and 3.40% when the template was on the non-target strand. On the bottom row of graphs, frequency of indels is shown for respective conditions.

FIG. 68 Demonstrates that retron mediated Precise editing is observed with Cas9 nickase in 4 component all-RNA system. Cas9 nickase activity in 4 component all-RNA system (RT mRNA+Cas9 mRNA+ncRNA/ncRNA-sgRNA+sgRNA) delivered to HEK293T cells by Lipofectamine MessengerMAX. All RNA was transfected at a fixed amount RT mRNA 100 ng, ncRNA/ncRNA-sgRNA 400 ng, Cas9 variant mRNA 100 ng, and sgRNA 5 ng. Either separated ncRNA or ncRNA-sgRNA fusion was used. Precise editing by Cas9 WT (Wild Type) was ˜9% when either ncRNA or ncRNA-sgRNA was used with additional sgRNA. With Cas9 H480A mutant that nicks non-target strand, activity was barely seen above background in any condition. With Cas9 D10A mutant that nicks target strand, precise editing was observed ˜0.1% frequency in both separated and fused ncRNA and sgRNA format. Indels are shown at the right graph at respective conditions of left graph. As expected, indels are extremely low for either nickases with or without additional sgRNA and only WT Cas9 generated significant indels.

FIG. 69. Demonstrates that retron mediated precise editing by Cas9 nickase is dependent on each component of all RNA system. Same as previous figure but testing negative controls that lack one of components (Cas9 or ncRNA/ncRNA-sgRNA or RT) of all RNA system with or without additional sgRNA. All control conditions tested show background precise editing activity in the top graph suggesting that precise editing achieved by Cas9 WT or Cas9 nickase is dependent on each component of all RNA system. Significant indels are detectable only with Cas9 WT in the bottom graph.

FIG. 70 demonstrates that dual sgRNAs increases precise editing, but not indels. The figure shows the results of testing dual sgRNAs for R2042 in all RNA system delivered to HEK293T cells by lipofection. At given amount of RT mRNA, Cas9 mRNA and ncRNA-sgRNA, additional sgRNA was added. The same ncRNA-sgRNA with template encoded a 25 bp insertion on the Cas9 sgRNA's non-targeting strand was used for all conditions. With one additional sgRNA (#1), precise editing was 0.27%, which increased to either 1.33% (#2) or 1.13% (#3) in the presence of a second sgRNA. On the bottom graph, frequency of indels is shown for respective conditions. See Example 9.

FIG. 71 Precise editing and indel results in HEK293T cells administered by lipofection an all-RNA retron editing system based on R6083 in various configurations. Configurations included testing different template lengths (25 bp-100 bp) with fused ncRNA-sgRNA constructs (with and with spiked sgRNA) and separated ncRNA and sgRNA constructs. At given amount of RT mRNA and Cas9 mRNA, ncRNA-sgRNA fusion with different template lengths ranging from 25 to 100 bp or ncRNA with 25 bp insertion were added. Precise insertion of 100 bp was observed at ˜2% efficiency. Separated ncRNA showed lower activity than ncRNA-sgRNA fusion with 25 bp insertion. The precise editing was dependent on the presence of RT and ncRNA as shown in top, right graph. On the bottom row of graphs, frequency of indels is shown for respective conditions.

FIG. 72 Results of testing R6083 retron to insert 25 bp template into AAVS1 locus in HEK293T cells. Retron (ncRNA-sgRNA fusion), retron RT, and Cas9 components were delivered as all RNA to HEK293T cells by lipofection. With additional sgRNA, 3% of editing efficiency was observed and the activity was completely dependent on the presence of either RT or ncRNA. On the right, frequency of indels is shown for respective conditions.

FIG. 73 Demonstrates that the separation of Cas9 and RT enzymes shows higher editing efficiency than when they are fused in retron editing with Eco3. Results of testing Cas9 and RT fusion for precise editing by Eco3 in all-RNA system delivered to HEK293T cells by Lipofectamine MessengerMAX. The same molar concentration of Cas9 or RT mRNA as in Cas9-RT fusion format were used in separation and the editing efficiency of fused or separated enzyme were compared for either Eco3 ncRNA (left on left graph) or Eco3 ncRNA-sgRNA fusion (right on right graph). With both ncRNA and ncRNA-sgRNA, separated Cas9 and RT achieved higher editing efficiency than when they were fused. Indels are shown at the right graph at respective conditions of left graph.

FIG. 74 Demonstrates that both cap and tail modifications to ncRNA significantly increased editing efficiency about 2-fold. Results of end protection of ncRNA-sgRNA fusion by cap and tail in 4 component all-RNA system (RT mRNA+Cas9 mRNA+ncRNA+sgRNA) delivered to HEK293T cells by Lipofectamine MessengerMAX. All RNA was transfected at a fixed amount RT mRNA 100 ng, ncRNA-sgRNA 400 ng, Cas9 mRNA 100 ng, and sgRNA 5 ng. ncRNA-gRNA fusion was either capped (+cap −tail) or poly-A tailed (−cap +tail) or both capped and poly-A tailed (+cap +tail). Using RNA without end protection (−cap −tail) produced ˜4.5% precise edits and the editing was dependent on retron since the absence of RT abrogated precise editing. While capping alone did not change editing efficiency, tailing alone slightly increased editing and both cap and tail significantly increased editing efficiency about 2-fold (left graph). Indels at all conditions were comparable in right graph.

FIG. 75 Schematics of ncRNA circularization which as demonstrated in FIG. 76 resulted in increase editing efficiency. Step 1. ncRNA is flanked by internal homology, intron and another homology sequences outward toward both ends. Step 2. Two homology sequences facilitate closer proximity of both ends. Exogenous GTP initiates a series of trans-esterification Step 3. introns are excised and ncRNA is ligated to a circular form.

FIG. 76 Results of testing modified ncRNAs (3′MS2 modification and circularized ncRNA) for precise editing in 4 component all-RNA system (RT mRNA+Cas9 mRNA+ncRNA+sgRNA) delivered to HEK293T cells by Lipofectamine MessengerMAX. All RNA was transfected at a fixed amount RT mRNA 100 ng, ncRNA 400 ng, Cas9 mRNA 100 ng, and sgRNA 5 ng. With the addition of MS2 stem loop at 3′ end of ncRNA, the activity nearly doubled at 15% efficiency from unmodified ncRNA (˜8%). Circularization of ncRNA achieved further increase of the efficiency, reaching editing efficiency of 22%. Circularization of ncRNA was done as described in FIG. 75. Without sgRNA, both precise edit and indels were not detectable as expected. Indels are shown at the right graph at respective conditions of left graph.

FIG. 77 Results suggest pairing between RT and its cognate ncRNA are required for precise editing. Results of pairing RT and ncRNA from same or different retrons for precise editing at EMX1 locus in HEK293T cells by all-RNA system. All RNA was transfected at a fixed amount RT mRNA 100 ng, ncRNA 400 ng, Cas9 mRNA 100 ng, and sgRNA 5 ng. At left graph, Aco1 RT was pared with either Eco3 ncRNA or its cognate Aco1 ncRNA (left panel). Eco3 RT was paired with Aco1 ncRNA or its cognate Eco3 ncRNA (right panel). Only when RT was paired with its cognate ncRNA, the system supported precise editing. Indels are shown at the right graph at respective conditions of left graph.

FIG. 78 Retron editing supports insertion of exon-sized long insertions of up to 305 bp. Results of testing exon-size long insertion at EMX1 locus with Aco1, Eco3 and R1262 retrons. Aco1 achieved precise insertions of 10, 100 and 205 bp at 8, 4, and 1.3% (left graph). Eco3 made precise insertions of 10 and 205 bp at 12 and 2.3%, respectively. A novel retron R1262 obtained 25, 205, and 305 bp insertion at 20, 15 and 8.8% efficiency. Indels are shown at the bottom graphs at respective conditions of top graphs.

FIG. 79 Demonstrates that novel retron R1262 inserts 205 bp insertion at AAVS1 at over 11% efficiency. Results of testing 205 bp insertion at AAVS1 locus with a novel retron R1262. R1262 obtained 25 and 205 bp insertion at 25%, and 11.1% efficiency respectively. As controls, precise editing was dependent on the presence of RT and sgRNA. Indels are shown at the right graph at respective conditions of left graph.

FIG. 80 Optimization of ratios of RT to ncRNA for precise editing by R1262 to install 205-305 bp insertions. Results of testing RT: ncRNA ratio for >200 bp long insertion at EMX1 locus with a novel reton R1262. RT: ncRNA ratio was tested at 1:12.5, 1:16.7 and 1:25 molar ratio. 1:16.7 ratio corresponds to 1:4 mass ratio that was used in the rest of studies. With ncRNA (right in the left graph), the best editing was observed at 1:12.5 ratio for both 205 and 305 bp insertion. With ncRNA-sgRNA fusion (left at left graph), all ratio tested generated comparable editing but a weak tendency of increased editing was seen with increased amount of ncRNA-sgRNA. Indels are shown at the right graph at respective conditions of left graph.

FIG. 81 NHEJ-based insertion of retron-made double strand DNA at target site A. Some retrons could produce double strand DNA using msR-msD hybrid as a primer (e.g. Sen1). Desired sequence to insert in the genome flanked by guide-RNA recognition sequence is integrated in msD of ncRNA. Both ends of double strand DNA produced by reverse transcription is trimmed by nuclease-guide RNA complex and inserted to the target site cleaved by nuclease-guide RNA complex. B. ncRNA is designed as in A to contain a sequence to insert at the target site flanked with guide-RNA recognition sequences. Second ncRNA includes a reverse complementary sequence of the first ncRNA in order that two of them form a duplex. Such double strand DNA is trimmed by nuclease-guide RNA complex and inserted to the target site cleaved by nuclease-guide RNA complex.

FIG. 82A Non-coding RNA optimization A. There are number of structural components in retron ncRNA, 1. a1/a2 inverted repeat 2. msR stem loops 3. msD stem loop 4. msR DNA. Fine-tuning strength of these structural elements by varying GC content, length and number of stem loops could enhance msD DNA production and consequent gene editing.

FIG. 82B Non-coding RNA optimization B. Separation of msR RT primer and msD RT template allows chemical modification of msR RT primer. In combination with end protection of msD RT template by cap and tail, the stability of ncRNA could be enhanced. Use of reverse complementary sequences at 3′ end of RT primer and 5′ end of msD RT template (depicted in dashed lines) stabilizes the primer/template complex.

FIG. 83A provides a schematic outline a modified retron gene editing system that comprises an engineered variant reverse transcriptase (RT) with improved fidelity and/or processivity. The engineered RT is produced by introducing selective amino acid substitutions into the sequence of a retron RT at residue positions that are orthologous to those altered residues in Murine Leukemia Virus (MMLV) RT that result in improved fidelity and processivity of MMLV RT. In Step (a), a structural alignment is constructed as between a retron RT and an engineered MMLV RT having one or more amino acid substitutions that are associated with improved fidelity and processivity in the MMLV RT context. In Step (b), the alignment is inspected to identify the orthologous amino acid residues in the retron RT that correspond with the substituted MMLV RT amino acids. Once identified, an engineered retron RT comprise one or more corresponding orthologous amino acid substitutions is constructed using routine recombinant engineering methodologies. In Step (c), unnecessary residues may be deleted optionally and based on the structural alignment information. In one embodiment, a retron gene editing system may comprise in (d) a ncRNA, a guide, a programmable nuclease (or an RNA encoding same), and the engineered RT (or an RNA encoding same). In Step (e), the components (which can be in all RNA format) are delivered to a cell (e.g., by LNP delivery), which results in (f) the targeted single (in the case of a nickase) or double strand cut, followed by (g) targeted repair by the reverse transcription product of the ncRNA at the cut site. This results in (h) DNA with a precise edit.

FIG. 83B provides a summary of mutations for installing in RTX3_1262 RT and RTX3_6242, as well as in Eco1 RT that are orthologous to known beneficial amino acid substitutions made in MMLV that are reported in the literature. In addition, the table provides for the reported phenotype change associated with the mutation in MMLV and the domain of the mutation.

FIG. 83C provides a structural alignment between RTX3_6342 RT (yellow) and MMLV-RT (Protein Data Bank 4MH8) (green), which reveals that the N-terminus of the RTX3-6342 RT may be able to be truncated, for example, at the suggested points of truncation.

FIG. 83D shows three dimensional structures of MMLV RT proximal to Q190 and the corresponding orthogonal positions Q161 in Eco1. In the top pictures, the glutamine is conserved in both MMLV and Eco1. The middle picture shows that the Q161 position in Eco1 is involved in nucleic acid contact. The lower shows that engineered MMLV variants having a Q190F mutation results in increased fidelity of around 2-fold. A corresponding substitution in Eco1 at Q161 (i.e., a Q161F mutation) may also result in increased fidelity of the Eco1 RT.

FIG. 83E Mutations in MMLV RT and Eco1 that improve processivity. Provides three dimensional structures of E302 of MMLV RT and the corresponding reside of G283 in Eco1, both of which appear to be involved in binding to nucleic acid. Residues in nucleic acid binding domains can be mutated to enforce stronger interactions. MMLV E302R creates a positive charge to interact with the negatively charged template. Eco1 G283 is in a conserved DNA binding alpha helix. Mutating G283 and orthologous retron residues to a positively charged amino acid may improve processivity.

FIG. 83F Mutations in MMLV RT and Eco1 that improve processivity. Provides three dimensional structures of T306 of MMLV RT and the corresponding reside of F287 in Eco1, both of which appear to be involved in binding to nucleic acid. Residues in nucleic acid binding domains can be mutated to enforce stronger interactions. MMLV T306K creates a positive charge to interact with the negatively charged template. Eco1 F287 is in a conserved DNA binding alpha helix. Mutating F287 and orthologous retron residues to a positively charged amino acid may improve processivity.

FIG. 84 describes the making of variant retron RTs by saturation mutagenesis of targeted domains involved in processivity and fidelity (e.g., palm, fingers, and thumb domains). An engineered RT is produced by introducing saturation mutagenesis into palm, finger, and thumb domains in a retron RT that are identified through a structural alignment with MMLV RT. In Step (a), a structural alignment is constructed as between a retron RT and an engineered MMLV RT having one or more amino acid substitutions that are associated with improved fidelity and processivity in the MMLV RT context, particularly in the palm, finger, and thumb domains. In Step (b), the palm, finger, and thumb domains are identified. In Step (c), the palm, finger, and/or thumb domains are mutagenized using a saturation mutagenis methodology and the resultant mutants are screened in an activity assay to select one or more leads variants that have increased processivity and/or fidelity. In Step (e), the components (which can be in all RNA format), including the lead retron RT variants, are delivered to a cell (e.g., by LNP delivery), which results in (f) the targeted single (in the case of a nickase) or double strand cut, followed by (g) targeted repair by the reverse transcription product of the ncRNA at the cut site. This results in (h) DNA with a precise edit.

FIG. 85A describes the making of variant retron RTs by constructing chimeric RT proteins that fuse a “Y region” of a retron RT (i.e., the region that is reported to be responsible for binding to the msr region of a retron ncRNA) with another RT (e.g, an MMLV RT) or replacing the “Y region” or one retron RT with that of another retron RT. In this way, the chimeric protein could be engineered against any particular ncRNA to have the corresponding Y region that would be expected to bind to that ncRNA.

FIG. 85B (SEQ ID NOS:19955-19968) (Top to Bottom) provides a multiple sequence alignment of amino acid sequences of numerous retron RTs (top). The boxed region indicates the position of a conserved VTG triplet that marks the N-terminal side of the Y region (as reported in Anna J Simon, Andrew D Ellington, Ilya J Finkelstein, Retrons and their applications in genome engineering, Nucleic Acids Research, Volume 47, Issue 21, 2 Dec. 2019, Pages 11007-11019, which is incorporated herein by reference), which is about 90 amino acids in length. The schematic below is a magnified version of the top figure.

FIG. 86 describes the making of variant retron RTs by fusing one or more processivity-enhancing factors, such as Sso7d and Sac7d, and/or one more fidelity-enhancing factors, e.g., 3′>5′ exonuclease domains to increase proofreading activity (e.g., POLE1, POLD1, POLG, Pfu, or KOD). In selecting appropriate factors to enhance the processivity and/or fidelity of retron RTs, reference is made Oscorbin et al., “The attachment of a DNA-binding Sso7d-like protein improves processivity and resistance to inhibitors of M-MuLV reverse transcriptase,” FEBS Lett, 594: 4338-4356 and Yarnall et al., “Drag-and-drop genome insertion of large sequences without double-strand DNA cleavage using CRISPR-directed integrases,” Nature Biotechnol, 2022.

FIG. 87 Electroporation using plasmid in K562 cells. Appropriate mixtures of plasmids were mixed and electroporated using a Neon Electroporation System into cells. After 72 hours incubation, genomic DNA was extracted and targeting region was amplified into sequencing libraries. Sequencing data was analyzed by CRISPResso2 and precise edit and indels were calculated.

FIG. 88 Results of plasmid-based assay in K562 cells demonstrating up to about 1˜10% precise edits and as low as 5˜30% indels with RTX003_2042, 6083v1, 6943, 1262, 6342L and 6342S. All are novel retrons and precise editing activity observed in this experiment strongly support that these novel retrons could generate msDNA inside K562 cells. These data replicate prior results seen in 293T cells suggesting that retron-mediated gene-editing is not impacted by cell-type specific effects.

FIG. 89 Results of plasmid-based assay comparing literature annotated retrons to novel retrons in K562 cells. These data demonstrate that novel retrons RTX3_1262, RTX3_6342L, and RTX3_6342S can generate about 15-19% precise edits and as low as 20˜35% indels. Novel retrons perform as well or better than Aco1 and better than the other literature validated retrons: Eco1, Eco3, Saul, and Sen1.

FIG. 90 Results of plasmid-based assay comparing RTX3_2781 to lead retrons RTX3_1262 and RTX3_6342S/L in K562 cells. These data demonstrate that RTX3_2781 installs precise edits at comparable efficiencies to RTX3_1262 and 6342S/L.

FIG. 91 Results of plasmid-based assay in K562 cells demonstrating up to about 0.6% precise edits and as low as 5% indels with RTX003_2042, 6083v1, 6943, 1262, 6342L and 6342S using a Cas9 D10A nickases. All are novel retrons and precise editing activity observed in this experiment strongly support that these novel retrons could generate msDNA inside cells can be used for nickase-initiated gene-editing.

FIG. 92 Results of plasmid-based assay in K562 cells demonstrating up to 0.5% precise edits using the LbCas12a nuclease. Precise editing in this system strongly suggests the generation of msDNA inside cells and compatibility with Cas12a-like nucleases for gene-editing.

FIG. 93 Results of plasmid-based assay in K562 cells demonstrating up to 0.5% precise edits using the TnpB nuclease. Precise editing in this system suggests the generation of msDNA inside cells and compatibility with TnpB-like nucleases for gene-editing.

FIG. 94 RTX3_6342 (predicted structure from alpha-fold)(yellow) aligned to MMLV-RT (PDB 4MH8) (green). Native RTX3_6342 RT is fused to a non-RT related domain at the N-terminus that may not be necessary for reverse transcription. Arrows mark potential truncation points of interest. Jumper et al. Highly accurate protein structure prediction with AlphaFold. Nature 596, 583-589 (2021). https://doi.org/10.1038/s41586-021-03819-2.

FIG. 95 Results of plasmid-based assay in K562 cells demonstrating up to about ˜20% precise edits and ˜20% indels with RTX003_6342S and truncation variants. Precise editing activity observed in this experiment strongly support that these truncations retain the capability to generate msDNA inside cells.

FIG. 96 Results of plasmid-based assay in K562 cells demonstrating up to ˜40% precise edits and ˜60% indels with RTX003_6342S and RTX3_1262 with differing insert sizes. Precise editing activity observed in this experiment suggest that RTX3_6342S can insert sequences of up to 405 bp into the EMX1 locus without a decline in precise editing. RTX3_1262 can insert up to 205 bp at the EMX1 locus before editing efficiencies decline. Substitutions in reads were ignored during CRISPResso2 analysis in this experiment.

FIG. 97 Results of plasmid-based assay in K562 cells demonstrating up to ˜40% precise edits and ˜40% indels with RTX003_6342S and RTX3_1262 with differing insert sizes while ignoring or quantifying substitutions during CRISPResso2 analysis. Precise editing activity observed in this experiment suggest that RTX3_6342S can insert sequences of up to 405 bp into the EMX1 locus but that RT fidelity could limit accurate installation of the intended edit (compare ignore (circles) vs count (triangles) substitutions).

FIG. 98 Results of plasmid-based assay in K562 cells screening mutations in RTX3_6342 reverse transcriptase predicted to interact with ncRNA after structural alignment of a predicted alphafold RTX3_6342 structure and a crystal structure of Retron Eco1 with ncRNA. Most mutations have modest effects on the installation of shorter 10 bp inserts but only two (N465K and N465R) potentially increase the installation frequencies of larger (305 bp) inserts.

FIG. 99 Results of plasmid-based assay in K562 cells demonstrating that orthologous MMLV mutations in RTX3_63425 do not consistently increase the installation frequencies of inserts >205 bp. MMLV (L139P)==RTX3_6342 (V265P), MMLV (T306K)==RTX3_6342 (M470K), MMLV (W313F)==RTX3_6342 (V477F).

FIG. 100 Results of plasmid-based assay in K562 cells screening mutations in RTX3_6342 reverse transcriptase predicted to interact with ncRNA after structural alignment of a predicted alphafold RTX3_6342 structure and a crystal structure of Retron Eco1 with ncRNA. The E238R, E479K and K255P are mutations that potentially increase the installation frequencies of 305 bp inserts at the EMX1 locus.

FIG. 101 Predicted structures of RTX3_6342 wildtype and with different N-terminal truncations. Predicted structures were generated with Alphafold and aligned to the Eco1/CryoEM structure (79VU.pdb) using PyMoL align.

FIG. 102 Results of plasmid-based assay in K562 cells assessing the effects of partial or full deletions of N-terminal helices of RTX3_6342 on the installation frequencies of longer inserts. We observed that partial N-terminal truncation boost the precise edits/indel ratio of longer inserts.

FIG. 103 Results of plasmid-based assay in K562 cells assessing the effects of DNA-binding domain fusion to RTX3_6342 on the installation frequencies of 405 bp inserts. Fusion of Sac7d or Sso7d DNA-binding domains to the N-terminus of retron reverse transcriptases led to two-fold (RTX3_6342S) or ten-fold (RTX3_1262) increases in installation frequencies of 405 bp inserts.

FIG. 104 Results of plasmid-based assay in K562 cells screening retron family members similar to RTX3_6342, 6083, 6943, or 1262. We noted that most retrons with robust gene-editing activity were in the RTX3_6342 family.

FIG. 105 Results of testing precise editing activity of a novel reton R2781 in all-RNA system. In FIG. 16, R2781 demonstrated 10 bp insertion at EMX1 locus in plasmid system, at similar activity as other hits, R1262, R6342S, 6342L. Here, the activity of R2781 for 25 bp, 205 bp and 405 bp insertion at EMX1 locus was evaluated with two different homology arm (HA) length (30 bp for both arms or 49 bp left/65 bp right). Precise insertion of 25 bp with 30 bp homology arm was achieved at 11% efficiency. The efficiency of same length insertion was reduced to half with longer homology arm (49/65 bp) suggesting that this retron reverse transcriptase might have limited enzymatic processivity. Conversely, the activity inserting 205 or 405 bp significantly dropped. Indels are shown at the right graph at respective conditions of left graph.

FIG. 106 Results of testing precise editing activity of a novel retron R6342S in all-RNA system with Cas.9 D10A. Previously, it was demonstrated that retron R6342S could achieve precise editing with Cas9 WT. Here, the activity for 25 bp insertion in EMX1 locus was evaluated in 293T cells with either Cas9 D10A or Cas9 D10A R221K/N394K mutant and either 10 or 50 ng sgRNA. Cas9 D10A demonstrated up to 0.4% editing while Cas9 D10A R221K/N394K showed higher activity up to 0.7% editing. These data demonstrate that retron-mediated genomic insertions are possible with Cas9 nickase as well as Cas9 WT which makes double-stranded breaks. Indels are shown at the right graph at respective conditions of left graph.

FIG. 107 Results of testing precise deletion activity of a novel reton R1262 in all-RNA system. In top panel, two deletion strategies are shown. Retron ncRNA is designed by juxtaposing left and right homology arms sequences to delete the intervening sequence. Dell is deleting 214 bp upstream of Cas9 cutting site and de12 is deleting 248 bp downstream of Cas9 cutting site at EMX1 locus. Retron mediated deletion was compared with direct delivery of increasing amount of single strand DNA (ssDNA) donor (150 and 300 ng). Bottom panel, left graph shows that R1262 was able to delete 248 bp (de12) from EMX1 locus at similar activity as ssDNA donor. Indels are shown at the right graph at respective conditions of left graph. Of note, unintended indel activity by retron-mediated deletion was ˜6 fold lower than ssDNA donor mediated deletion. Deletion activity of 214 bp (del1) by R1262 was not detected

FIG. 108 Results of testing non-homologous end joining (NHEJ) based insertion activity of a novel reton R1262 and R6342 in all-RNA system. At left, the strategy to derive double strand DNA (dsDNA), which is the substrate of NHEJ mechanism is depicted. 1. Retron reverse transcriptase generates complementary single strand DNAs from two ncRNAs that contain either sense or anti-sense insert sequences. 2. Complementary single strand DNAs form duplex 3. Cas9-sgRNA complex remove Y-shape single stranded portion (msR and msD spacer sequences) at both extremes. 4. Cas9 trimmed blunt-end double strand DNA is successfully inserted at target site. The inclusion of sgRNA recognition sequences (same as genomic target sequence) in tandem at both ends of insert leads to blunt-ended double strand DNA and only one direction of insertion allows more stable integration by preventing from recutting by Cas9. Using this strategy, R1262 with two complementary ncRNAs integrated ˜120 bp dsDNA on EMX1 locus at ˜1% efficiency, higher when insert sequence is flanked with two sgRNA sequences in tandem (right, top graph). Basal insertion activity of R6342 without tandem sgRNA sequences was slightly higher than that of R1262. Indels are shown at the right, bottom graph at respective conditions of top graph.

FIG. 109 Results of testing immune response to retron RNAs. Human peripheral blood mononuclear cells (hPBMCs) were used to evaluate immune response since PBMCs contain both innate and adaptive immune cells and they are equipped with sensors to detect foreign nucleic acids. Frozen hPBMCs were thawed to rest for overnight. RT mRNA with U or m1′P and ncRNA with/without cap0 (m7G) at 5′ end were electroporated individually or together as indicated in the label, along with unmodified GFP mRNA from TriLink as a control. After overnight culture, the supernatant was analyzed for cytokine production. Among 25 cytokines and chemokines examined, those detected above detection limit are shown. Type I interferon, a hallmark of immune response to foreign nucleic acids was not detected in any retron RNA transfected cells and some inflammatory cytokines/chemokines were detected at low level in retron RNA transfected cells, but at much lower level than control GFP mRNA except RT mRNA without U modification.

FIG. 110 Results of testing retron-mediated gene editing in human stem progenitor cells (HSPCs) in all-RNA system. Bone marrow derived CD34+ human stem progenitor cells (HSPCs) were used to evaluate the performance of retron-mediated gene editing in primary cells. Frozen HSPCs were thawed to expand for three days in the presence of hSCF, hFLT3-L, hTPO cytokines to prevent differentiation. Cas9 mRNA, guide RNA (gRNA), R6342 RT mRNA and ncRNA was mixed at mass ratio indicated in each label, and electroporated to HSPCs by Lonza electroporator using two different programs. After additional three-day culture, genomic DNA was extracted and sequenced to measure editing frequency. With total of 5 microgram of RNA, divided at 1:0.3:4:1=Cas9:gRNA:ncRNA:RT, precise insertion of 25 bp at AAVS1 locus was observed at 0.1% of frequency (left graph). Indels at respective condition is shown at the right graph.

FIG. 111 Results of testing retron-mediated gene editing in human T-cells in all-RNA system. Human pan T-cells from peripheral blood were used to evaluate the performance of retron-mediated gene editing in primary cells. Frozen T-cells were thawed and activated for two days in the presence of anti-CD3/anti-CD28 conjugated magnetic beads and IL-2 cytokine. Cas9 mRNA, guide RNA (gRNA), R6342 RT mRNA and ncRNA was mixed at mass ratio indicated in each label, and electroporated to T-cells by either Neon or Lonza electroporator. After additional three-day culture, genomic DNA was extracted and sequenced to measure editing frequency. With total of 3 microgram of RNA, divided at 1:0.3:3:1=Cas9:gRNA:ncRNA:RT, precise insertion of 25 bp at AAVS1 locus was observed up to 1.7% of frequency using Neon machine (left graph). Indels in their respective condition is shown at the right graph.

FIG. 112 provides a schematic of retron ncRNA library. Variants of different elements in the ncRNA (modified a1:a2 stem, msR, msD regions) are associated with unique barcodes and the library is synthesized as an oligo library. By sequencing barcodes inserted at the genomic locus, the efficiency of associated ncRNA variants can be measured in human cells.

FIG. 113 outlines the variant library of Example 31.

FIG. 114 provides a schematic depicting the screening process used to screen the ncRNA variant library described in Example 31.

FIG. 115 provides a scatter plot summarizing the results of FIG. 31. In the scatter plot, each dot represents one variant with relative genomic insertion level on the y-axis and ssDNA production level on the x-axis. Dotted lines represent the levels of genomic insertion and ssDNA production from WT R6342S control. Hence, the ncRNA variants on the upper right part of the dotted lines are outperforming WT R6342S control on both levels. As can be seen, most of these variants have either a1a2 or msD stem elements. We list 13 such variants that show over 1.5-fold over WT R6342S control on both genomic insertion and ssDNA production level.

### DEFINITIONS

All technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & 62/1005 Marham, The Harper Collins Dictionary of Biology (1991).

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

It must be noted that as used herein and in the appended claims, the singular forms “a,” “an,” and “the” include plural referents unless the context clearly dictates otherwise. Thus, for example, reference to “a ncRNA” includes a plurality of ncRNAs and reference to “the reverse transcriptase” includes reference to one or more RTs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. For example, claims may be drafted to exclude certain RT sequences.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub combination was individually and explicitly disclosed herein.

### Biologically Active

As used herein, the term “biologically active” refers to a characteristic of an agent (e.g., DNA, RNA, or protein) that has activity in a biological system (including in vitro and in vivo biological system), and particularly in a living organism, such as in a mammal, including human and non-human mammals. For instance, an agent when administered to an organism has a biological effect on that organism, is considered to be biologically active.

### Bulge

As used herein, the term “bulge” refers to a small region of unpaired base(s) that interrupts a “stem” of base-paired nucleotides. The bulge may comprise one or two single-stranded or unbase-paired nucleotides joined at both ends by base-paired nucleotides of the stem. The bulge can be symmetrical (viz., the two unbase-paired single-stranded regions have the same number of nucleotides), or asymmetrical (viz., the unbase-paired single stranded region(s) have different or unequal numbers of nucleotides), or there is only one unbase-paired nucleotide on one strand. A bulge can be described as A/B (such as a “2/2 bulge,” or a “1/0 bulge”) wherein A represents the number of unpaired nucleotides on the upstream strand of the stem, and B represents the number of unpaired nucleotides on the downstream strand of the stem. An upstream strand of a bulge is more 5′ to a downstream strand of the bulge in the primary nucleotide sequence. cDNA

As used hereing, the term “cDNA” refers to a strand of DNA copied from an RNA template, e.g., by a reverse transcriptase.

### Cognate

The term “cognate” refers to two biomolecules that normally interact or co-exist in nature.

### Complementary

As used herein, the terms “complementary” or “substantially complementary” are meant to refer to a nucleic acid (e.g., RNA, DNA) that comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine (A) pairing with thymidine (T), adenine (A) pairing with uracil (U), and guanine (G) pairing with cytosine (C) [DNA, RNA]. In addition, for hybridization between two RNA molecules (e.g., dsRNA), and for hybridization of a DNA molecule with an RNA molecule (e.g., when a DNA target nucleic acid base pairs with a guide RNA, etc.): guanine (G) can also base pair with uracil (U). For example, G/U base-pairing is at least partially responsible for the degeneracy (i.e., redundancy) of the genetic code in the context of tRNA anti-codon base-pairing with codons in mRNA. Thus, in the context of this disclosure, a guanine (G) is considered complementary to both a uracil (U) and to an adenine (A). For example, when a G/U base-pair can be made at a given nucleotide position of a dsRNA duplex of a guide RNA molecule, the position is not considered to be non-complementary, but is instead considered to be complementary.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a bulge, a loop structure or hairpin structure, etc.). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656), the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489), and the like.

### DNA-Guided Nuclease

As used herein, an “DNA-guided nuclease” is a type of “programmable nuclease,” and a specific type of “nucleic acid-guided nuclease.” An example of a DNA-guided nuclease is reported in Varshney et al., DNA-guided genome editing using structure-guided endonucleases, Genome Biology, 2016, 17(1), 187, which may be used in the context of the present disclosure and is incorporated herein by reference. As used herein, the term “DNA-guided nuclease” or “DNA-guided endonuclease” refers to a nuclease that associates covalently or non-covalently with a guide RNA thereby forming a complex between the guide RNA and the DNA-guided nuclease. The guide RNA comprises a spacer sequence which comprises a nucleotide sequence having complementarity with a strand of a target DNA sequence. Thus, the DNA-guided nuclease is indirectly guided or programmed to localize to a specific site in a DNA molecule through its association with the guide RNA, which directly binds or anneals to a strand of the target DNA through its complementarity region via Watson-Crick base-pairing.

### DNA Regulatory Sequences

As used herein, the terms “DNA regulatory sequences,” “control elements,” and “regulatory elements,” can be used interchangeably herein to refer to transcriptional and translational control sequences, such as promoters, enhancers, polyadenylation signals, terminators, protein degradation signals, and the like, that provide for and/or regulate transcription of a non-coding sequence (e.g., guide RNA) or a coding sequence and/or regulate translation of a mRNA into an encoded polypeptide.

### Donor Nucleic Acid

By a “donor nucleic acid” or “donor polynucleotide” or “donor DNA” or “HDR donor DNA” it is meant a single-stranded DNA to be inserted at a site cleaved by a programmable nuclease (e.g., a CRISPR/Cas effector protein; a TALEN; a ZFN; a meganuclease) (e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g., within about 200 bases or less of the target site, e.g., within about 190 bases or less of the target site, e.g., within about 180 bases or less of the target site, e.g., within about 170 bases or less of the target site, e.g., within about 160 bases or less of the target site, e.g., within about 150 bases or less of the target site, e.g., within about 140 bases or less of the target site, e.g., within about 130 bases or less of the target site, e.g., within about 120 bases or less of the target site, e.g., within about 110 bases or less of the target site, e.g., within about 100 bases or less of the target site, e.g., within about 90 bases or less of the target site, e.g., within about 80 bases or less of the target site, e.g., within about 70 bases or less of the target site, e.g., within about 60 bases or less of the target site, e.g., 50 bases or less of the target site, e.g., within about 30 bases, within about 15 bases, within about 10 bases, within about 5 bases, or immediately flanking the target site, to support homology-directed repair between it and the genomic sequence to which it bears homology.

### Encodes

As used herein, a DNA sequence that “encodes” a particular RNA is a DNA nucleotide sequence that is transcribed into RNA. A DNA polynucleotide may encode an RNA (mRNA) that is translated into protein (and therefore the DNA and the mRNA both encode the protein), or a DNA polynucleotide may encode an RNA that is not translated into protein (e.g. tRNA, rRNA, microRNA (miRNA), a “non-coding” RNA (ncRNA), a guide RNA, etc.). In the case of retrons, the retron DNA may encode the ncRNA loci (which includes the msr and msd regions) as well as a retron RT.

### Engineered Retron

As used herein, the term “engineered retron” or equivalently, “recombinant retron,” refers to a retron that does not occur in nature. In one embodiment, engineered retrons can include wildtype or naturally-occurring retrons that are modified to contain at least one modification, including a single nucleotide substitution, insertion, or deletion, or a substitution, insertion, or deletion of more than one nucleotide, i.e., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or up to 100, or up to 200, 300, 400, 500, 600, 700, 800, 900, 1000, 1100, 1200, 1300, 1400, 1500, 1600, 1700, 1800, 1900, or up to 2000 nucleotides substituted, inserted, or deleted from a starting point retron (e.g., a wildtype retron). Where more than one nucleotide of a starting point retron (e.g., a wildtype retron) is substituted, deleted, or inserted, the nucleotides may be contiguous or non-contiguous. While an engineered retron as a whole is not naturally-occurring, it may include components such as nucleotide sequences that do occur in nature. For example, an engineered retron can have nucleotide sequences from different organisms (e.g., from different bacteria species), or from completely synthetic/artificial/recombinant nucleic acid sequences. Thus, an engineered retron can have a bacterial nucleotide sequence, a human nucleotide sequence, a viral nucleotide sequence, and/or a synthetic/artificial/recombinant nucleotide sequence, and/or combinations of such sequences. An example of modifications of the recombinant retrons disclosed herein include the insertion of a heterologous nucleic acid sequence in a retron, for example, inserted into the ncRNA locus, such as in the msr or the msd loci. Linking guide RNA molecules to the 5′ and/or 3′ ends (i.e., linking one at the 5′ end of a ncRNA and/or one at the 3′ end of a ncRNA) also represent another modification envisioned by the recombinant retrons disclosed herein. In such embodiments, the guide RNA molecules may also be categorized or referred to more generally as types of heterologous nucleic acid sequences used to modify starting point retrons.

### Exosomes

As used herein, the term “exosomes” refer to small membrane bound vesicles with an endocytic origin. Without wishing to be bound by theory, exosomes are generally released into an extracellular environment from host/progenitor cells post fusion of multivesicular bodies the cellular plasma membrane. As such, exosomes can include components of the progenitor membrane in addition to designed components (e.g. engineered retron). Exosome membranes are generally lamellar, composed of a bilayer of lipids, with an aqueous inter-nanoparticle space.

### Expression Vector

As used herein, the term “expression vector” or “expression construct” refers to a vector that includes one or more expression control sequences, and an “expression control sequence” is a DNA sequence that controls and regulates the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available, such as from Novagen (Madison, WI), Clontech (Palo Alto, CA), Stratagene (La Jolla, CA), and Invitrogen/Life Technologies (Carlsbad, CA). The present invention comprehends recombinant vectors that may include viral vectors, bacterial vectors, protozoan vectors, DNA vectors, or recombinants thereof.

### Guide RNA

The RNA molecule that binds to a programmable nuclease of a retron-based gene editing systems and which targets the nuclease to a specific location within the targeted polynucleotide sequence is referred to herein as the “guide RNA” or “guide RNA polynucleotide” (also referred to herein as a “guide RNA” or “gRNA” or “crRNA”). In certain embodiments (depending on the particular nuclease to which it interacts with), a guide RNA comprises two segments, a “DNA-targeting segment” and a “protein-binding segment.” By “segment” it is meant a segment/section/region of a molecule, e.g., a contiguous stretch of nucleotides in an RNA. As an illustrative, non-limiting example, a protein-binding segment of a guide RNA can comprise base pairs 5-20 of the RNA molecule that is 40 base pairs in length; and the DNA-targeting segment can comprise base pairs 21-40 of the RNA molecule that is 40 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and may include regions of RNA molecules that are of any total length and may or may not include regions with complementarity to other molecules.

The DNA-targeting segment (or “DNA-targeting sequence”) comprises a nucleotide sequence that is complementary to a specific sequence within a targeted polynucleotide sequence (the complementary strand of the targeted polynucleotide sequence) designated the “protospacer-like” sequence herein. The protein-binding segment (or “protein-binding sequence”) interacts with a site-directed modifying polypeptide. When the site-directed modifying polypeptide is a CRISPR nuclease, site-specific cleavage of the targeted polynucleotide sequence may occur at locations determined by both (i) base-pairing complementarity between the guide RNA and the targeted polynucleotide sequence; and (ii) a short motif (referred to as the protospacer adjacent motif (PAM)) in the targeted polynucleotide sequence.

### Heterologous Nucleic Acid Sequence

As used herein, the term “heterologous nucleic acid” refers to a genotypically distinct entity from that of the rest of the entity to which it is compared or into which it is introduced or incorporated. For example, a polynucleotide introduced by genetic engineering techniques into a different cell type is a heterologous polynucleotide (e.g., DNA or RNA) and, if expressed, can encode a heterologous polypeptide. Similarly, a cellular sequence (e.g., a gene or portion thereof) that is incorporated into a viral vector is a heterologous nucleotide sequence with respect to the vector. In some embodiments, the heterologous sequence inserted into the wild-type retron regions does not naturally insert into such regions (e.g., the engineered retron with the inserted heterologous sequence is not naturally existing). For example, the heterologous sequence can be from the same species of bacteria in which the wild-type retron is normally found, so long as the heterologous sequence is not naturally inserted in the wild-type retron at the location in which the heterologous sequence is inserted. In certain embodiments, the heterologous sequence is a mammalian sequence (e.g., a human sequence), or a reverse complement thereof. Heterologous nucleic acid sequences introduced into retrons can including without limitation guide RNA sequences, HDR donor templates, protein-encoding genes, or non-coding functional RNA elements (e.g., stem-loops, hairpins, and bulges).

### Homology-Directed Repair

As used herein, “homology-directed repair (HDR)” refers to the specialized form DNA repair that takes place, for example, during repair of double-strand breaks in cells. This process requires nucleotide sequence homology, uses a “donor” molecule to template repair of a “target” molecule (i.e., the one that experienced the double-strand break), and leads to the transfer of genetic information from the donor to the target. Homology-directed repair may result in an alteration of the sequence of the target molecule (e.g., insertion, deletion, mutation), if the donor polynucleotide differs from the target molecule and part or all of the sequence of the donor polynucleotide is incorporated into the targeted polynucleotide sequence.

### Identical

As used herein, the term “identical” refers to two or more sequences or subsequences which are the same. In addition, the term “substantially identical,” as used herein, refers to two or more sequences which have a percentage of sequential units which are the same when compared and aligned for maximum correspondence over a comparison window, or designated region as measured using a comparison algorithm or by manual alignment and visual inspection. By way of example only, two or more sequences may be “substantially identical” if the sequential units are about 60% identical, about 65% identical, about 70% identical, about 75% identical, about 80% identical, about 85% identical, about 90% identical, or about 95% identical over a specified region. Such percentages to describe the “percent identity” of two or more sequences. The identity of a sequence can exist over a region that is at least about 75-100 sequential units in length, over a region that is about 50 sequential units in length, or, where not specified, across the entire sequence. This definition also refers to the complement of a test sequence.

Alternatively, substantially identical or similarity exists when a nucleic acid or fragment thereof hybridizes to another nucleic acid, to a strand of another nucleic acid, or to the complementary strand thereof, under stringent hybridization conditions. “Stringent hybridization conditions” and “stringent wash conditions” in the context of nucleic acid hybridization experiments depend upon a number of different physical parameters. Nucleic acid hybridization will be affected by such conditions as salt concentration, temperature, solvents, the base composition of the hybridizing species, length of the complementary regions, and the number of nucleotide base mismatches between the hybridizing nucleic acids, as will be readily appreciated by those skilled in the art. One having ordinary skill in the art knows how to vary these parameters to achieve a particular stringency of hybridization.

### Lipid Nanoparticle (LNP)

As used herein, the term “lipid nanoparticle” or LNP refers to a type of lipid particle delivery system formed of small solid or semi-solid particles possessing an exterior lipid layer with a hydrophilic exterior surface that is exposed to the non-LNP environment, an interior space which may aqueous (vesicle like) or non-aqueous (micelle like), and at least one hydrophobic inter-membrane space. LNP membranes may be lamellar or non-lamellar and may be comprised of 1, 2, 3, 4, 5 or more layers. In some embodiments, LNPs may comprise a nucleic acid (e.g. engineered retron) into their interior space, into the inter membrane space, onto their exterior surface, or any combination thereof. In some embodiments, an LNP of the present disclosure comprises an ionizable lipid, a structural lipid, a PEGylated lipid (aka PEG lipid), and a phospholipid. In alternative embodiments, an LNP comprises an ionizable lipid, a structural lipid, a PEGylated lipid (aka PEG lipid), and a zwitterionic amino acid lipid.

Further discuss of liposomes can be found, for example, in Tenchov et al., “Lipid Nanoparticles—From Liposomes to mRNA Vaccine Delivery, a Landscape of Diversity and Advancement,” ACS Nano, 2021, 15, pp. 16982-17015 (the contents of which are incorporated by reference).

### Linker

As used herein, the term“linker” refers to a molecule linking or joining two other molecules or moieties. The linker can be an amino acid sequence in the case of a linker joining two fusion proteins. For example, an RNA-guided nuclease (e.g., Cas12a) can be fused to a retron reverse transcriptase by an amino acid linker sequence. The linker can also be a nucleotide sequence in the case of joining two nucleotide sequences together. For example, in the instant case, a ncRNA at its 5′ and/or 3′ ends may be linked by a nucleotide sequence linker to one or more guide RNAs. In other embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

### Liposomes

As used herein, the term “liposomes” refer to a type of lipid particle delivery system comprising small vesicles that contain at least one lipid membrane surrounding an aqueous inner-nanoparticle space that is generally not derived from a progenitor/host cell. Liposomes are a versatile carrier platform in that they are capable of transporting hydrophobic or hydrophilic molecules, including small molecules, proteins, and nucleic acids into cells. They were the earliest developed generation of nanoscale medicine delivery platform. Numerous liposomal drug formulations have been approved for human medicines, e.g., Doxil, a lipid nanoparticle formulation of the antitumor agent doxorubicin. Further discuss of liposomes can be found, for example, in Tenchov et al., “Lipid Nanoparticles—From Liposomes to mRNA Vaccine Delivery, a Landscape of Diversity and Advancement,” ACS Nano, 2021, 15, pp. 16982-17015 (the contents of which are incorporated by reference).

### Loop

As used herein, the term “loop” in a polynucleotide refers to a single stranded stretch of one or more nucleotides, such as 2, 3, 4, 5, 6, 7, 8, 9, or 10 nucleotides, wherein the most 5′ nucleotide and the most 3′ nucleotide of the loop are each linked to a base-paired nucleotide in a stem.

### Micelles

As used herein, the term “micelles” refer to small particles which do not have an aqueous intra-particle space.

### Nanoparticle

As used herein, the term “nanoparticle” refers to any nanoscale particle typically ranging in size from about 1 nm to 1000 nm.

### Nuclear Localization Sequence (NLS)

As used herein, the term“nuclear localization sequence” or “NLS” refers to an amino acid sequence that promotes import of a protein (e.g., a RNA-guided nuclease) into the cell nucleus, for example, by nuclear transport. Nuclear localization sequences are known in the art. For example, NLS sequences are described in Plank et al., international PCT application, PCT/EP2000/011690, filed Nov. 23, 2000, published as WO/2001/038547 on May 31, 2001, the contents of which are incorporated herein by reference for its disclosure of exemplary nuclear localization sequences.

### Nucleic Acid

As used herein, the term “nucleic acid” or “nucleic acid molecule” or “nucleic acid sequence” or “polynucleotide” generally refer to deoxyribonucleic or ribonucleic oligonucleotides in either single- or double-stranded form. The term may also encompass oligonucleotides containing known analogues of natural nucleotides. The term also may also encompass nucleic acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. The term encompasses both ribonucleic acid (RNA) and DNA, including cDNA (including RT DNA), genomic DNA, synthetic, synthesized (e.g., chemically synthesized) DNA, and/or DNA (or RNA) containing nucleic acid analogs. The nucleotides Adenine (A), Thymine (T), Guanine (G) and Cytosine (C) also may (or may not) encompass nucleotide modifications, e.g., methylated and/or hydroxylated nucleotides, e.g., Cytosine (C) encompasses 5-methylcytosine and 5-hydroxymethylcytosine.

### Nucleic Acid-Guided Nuclease

As used herein, the term “nucleic acid-guided nuclease” or “nucleic acid-guided endonuclease” refers to a nuclease that associates covalently or non-covalently with a guide nucleic acid (e.g., a guide RNA or a guide DNA) thereby forming a complex between the guide nucleic acid and the nucleic acid-guided nuclease. The guide nucleic acid comprises a spacer sequence which comprises a nucleotide sequence having complementarity with a strand of a target DNA sequence. Thus, the nucleic acid-guided nuclease is indirectly guided or programmed to localize to a specific site in a DNA molecule through its association with the guide nucleic acid, which directly binds or anneals to a strand of the target DNA through its complementarity region via Watson-Crick base-pairing. In some embodiments, the nucleic acid-guided nuclease will include a DNA-binding activity (e.g., as in the case for CRISPR Cas9). Most commonly, the nucleic acid-guided nuclease is programmed by associating with a guide RNA molecule and in such cases the nuclease may be called “RNA-guided nuclease.” When programmed by a guide DNA, the nuclease may be called a “DNA-guided nuclease.” Nucleic acid-guided, RNA-guided, or DNA-guided nucleases may also be referred to as “programmable nucleases,” which also include other classes of programmable nucleases which associate with specific DNA sequences through amino acid/nucleotide sequence recognition (e.g., zinc fingers nucleases (ZFN) and transcription activator like effector nucleases (TALEN)) rather than through guide RNAs. In addition, any nuclease contemplated herein may also be engineered to remove, inactivate, or otherwise eliminate one or more nuclease activities (e.g., by introducing a nuclease-inactivating mutation in the active site(s) of a nuclease). A nuclease that has been modified to remove, inactivate, or otherwise eliminate all nuclease activity may be referred to as a “dead” nuclease. A dead nuclease is not able to cut either strand of a double-stranded DNA molecule. A nuclease that has been modified to remove, inactivate, or otherwise eliminate at least one nuclease activity but which still retains at least one nuclease activity may be referred to as a “nickase” nuclease. A nickase nuclease cuts one strand of a double-stranded DNA molecule, but not both strands. For example, a CRISPR Cas9 naturally comprises two distinct nuclease activity domains, namely, the HNH domain and the RuvC domain. The HNH domain cuts the strand of DNA bound to the guide RNA and the RuvC domain cuts the protospacer strand. One can obtain a nickase Cas9 by inactivating either the HNH domain or the RuvC domain. One can obtain a dead Cas9 by inactivating both the HNH domain and the RuvC domain. Other RNA-guided nuclease may be similarly converted to nickases and/or dead nucleases by inactivating one or more of the existing nuclease domains.

### Operably Linked

As used herein, the term “operably linked” or “under transcriptional control,” when used in conjunction with the description of a promoter, refers to the correct location and orientation in relation to a polynucleotide (e.g., a coding sequence) to control the initiation of transcription by RNA polymerase and expression of the coding sequence, such as one for the msr gene, msd gene, and/or the ret gene. Other transcriptional control regulatory elements (e.g., enhancer sequences, transcription factor binding sites) may also be operably linked to a gene if their location relative to a gene controls or regulates the expression of the gene.

### Programmable Nuclease

As used herein, the term “programmable nuclease” is meant to refer to a polypeptide that has the property of selective localization to a specific desired nucleotide sequence target in a nucleic acid molecule (e.g., to a specific gene target) due to one or more targeting functions. Such targeting functions can include one or more DNA-binding domains, such as zinc finger domains characteristic of many different types of DNA binding proteins or TALE domains characteristic of TALEN proteins. Such targeting function may also include the ability to associate and/or form a complex with a guide RNA, which then localizes to a specific site on the DNA which bears a sequence that is complementary to a portion of the guide RNA (i.e., the spacer of the guide RNA). In some embodiments, the programmable nuclease may be a single protein which comprises both a domain that binds directly (e.g., a ZF protein) or indirectly (e.g., an RNA-guided protein) to a target DNA site, as well as a nuclease domain. In other embodiments, the programmable nuclease may be a composite of two or more separate proteins or domains (from different proteins) which together provide the necessary functions of selective DNA binding and nuclease activity. For example, the programmable nuclease may comprise a (a) nuclease-inactive RNA-guided nuclease (which still is capable of binding a guide RNA, localizing to a target DNA, and binding to the target DNA, but not capable of cutting or nicking the strands) fused to a (b) nuclease protein or domain, such as a FokI nuclease.

### Promoter

As used herein, the term“promoter” is art-recognized and refers to a nucleic acid molecule with a sequence recognized by the cellular transcription machinery and which is able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive expression by the various vectors of the present disclosure.

### Recombinant Nucleic Acid

A “recombinant nucleic acid” or “recombinant nucleotide” refers to a molecule that is constructed by joining nucleic acid molecules, which optionally may self-replicate in a live cell.

### Retron

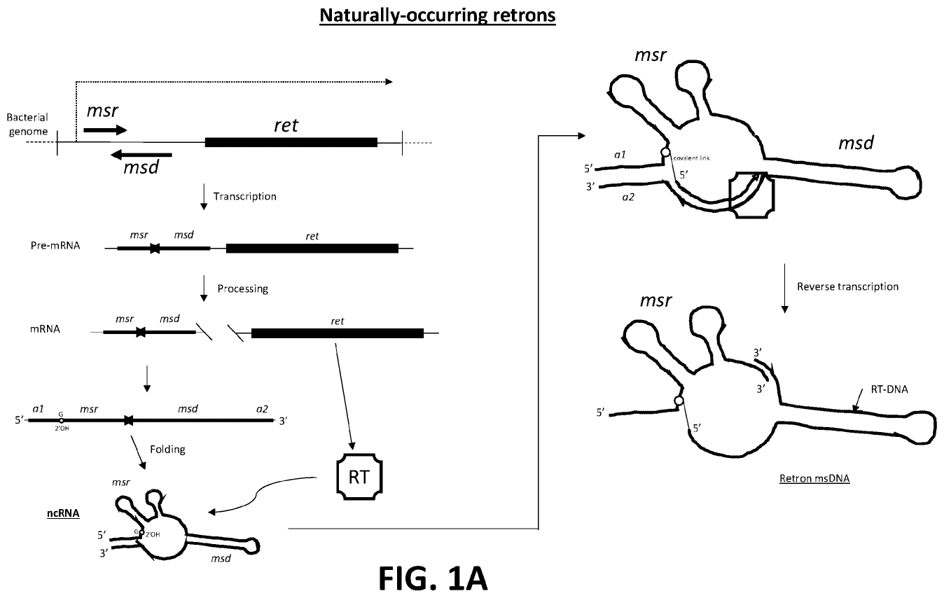

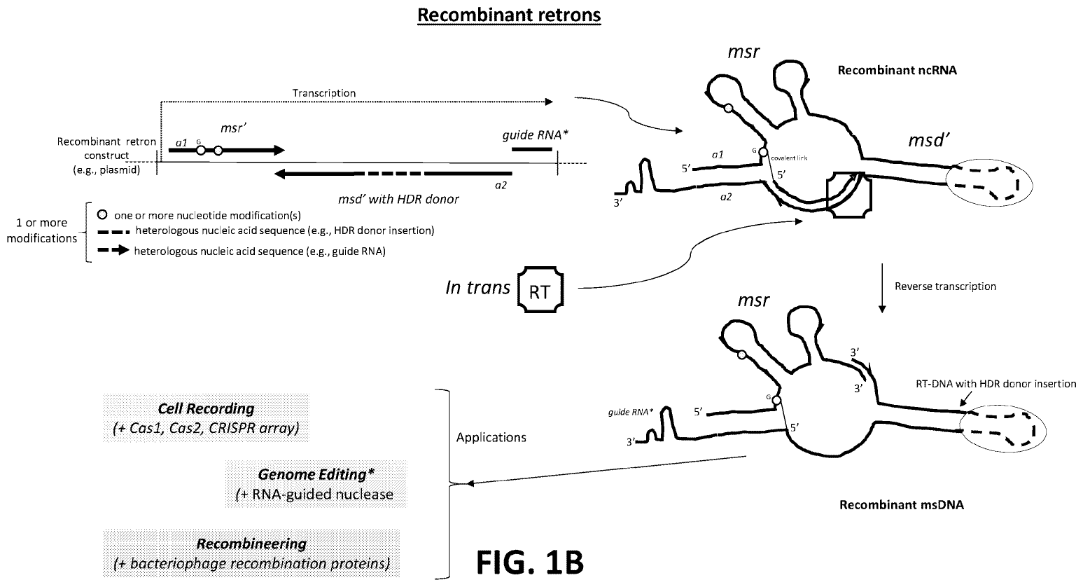

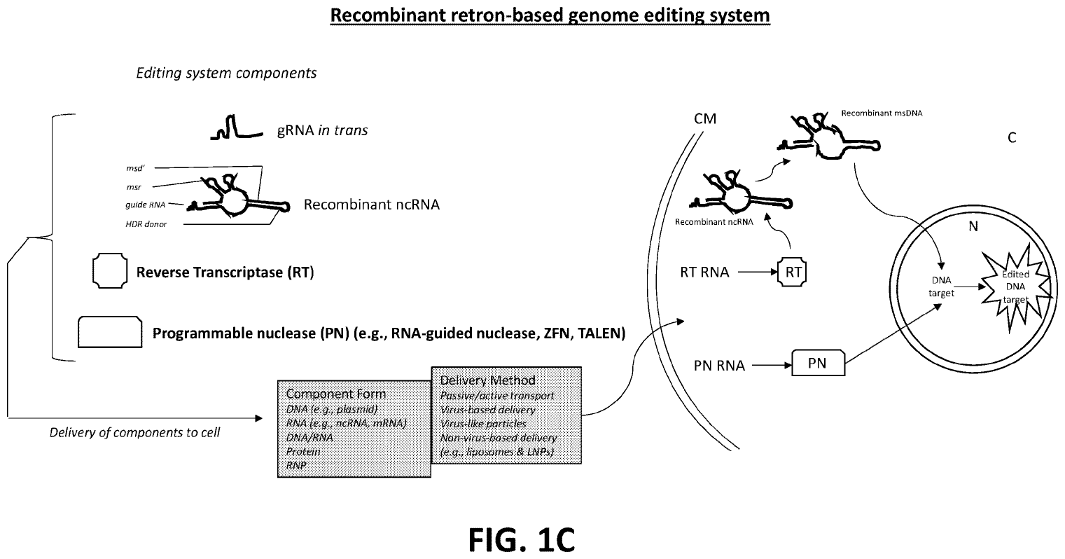

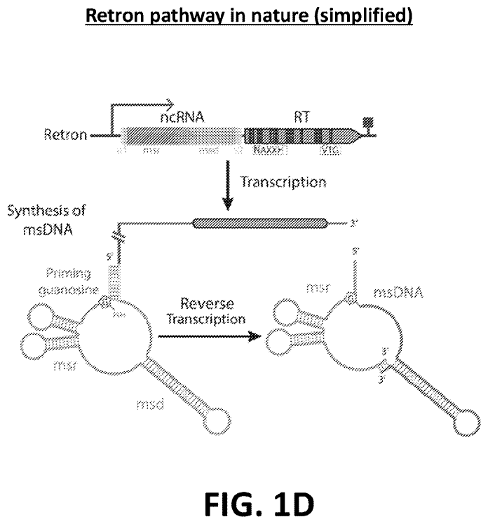

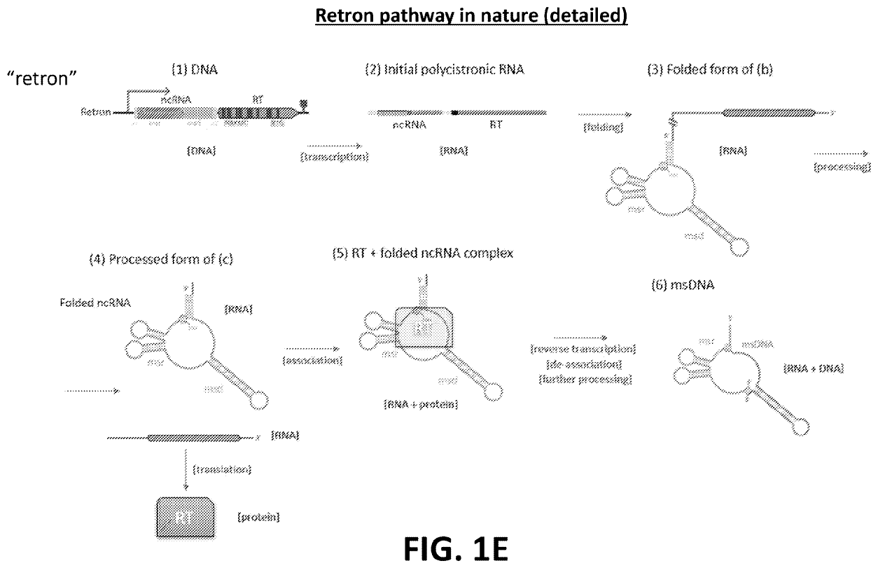

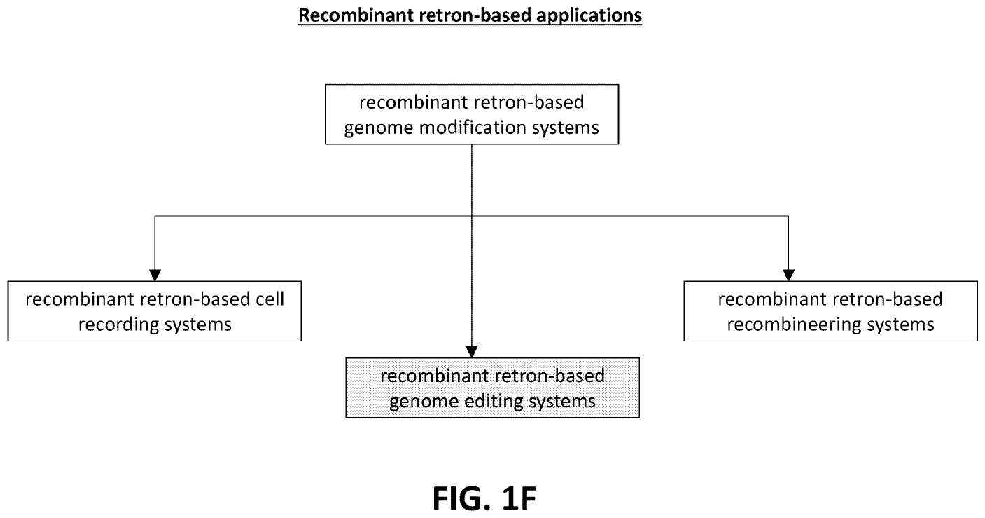

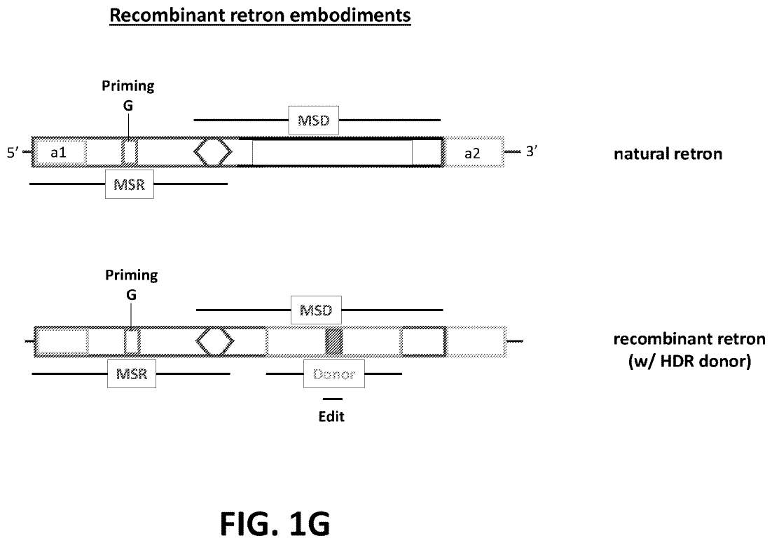

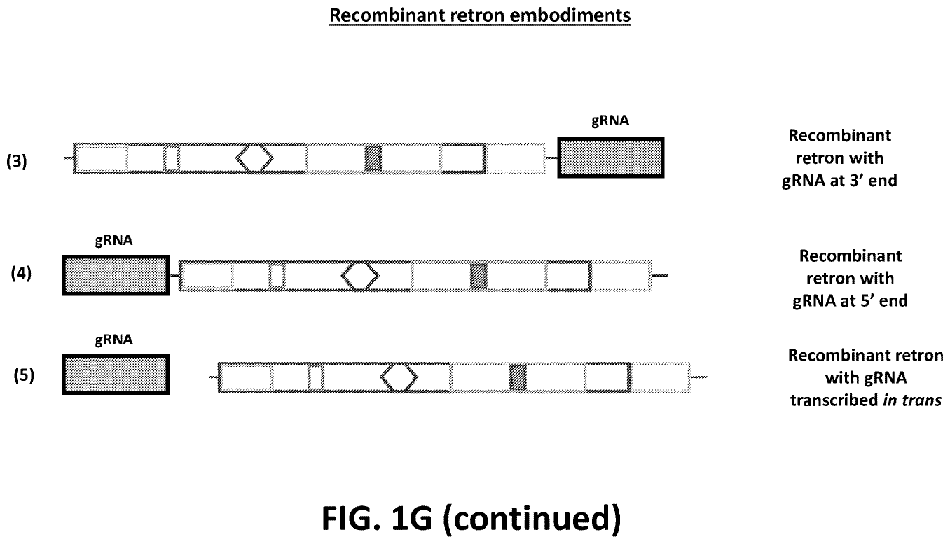

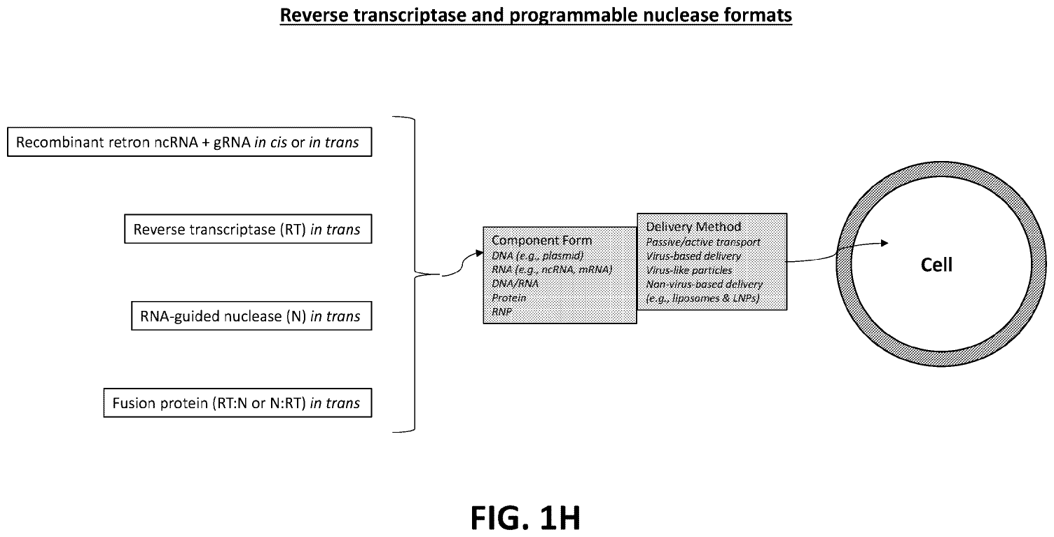

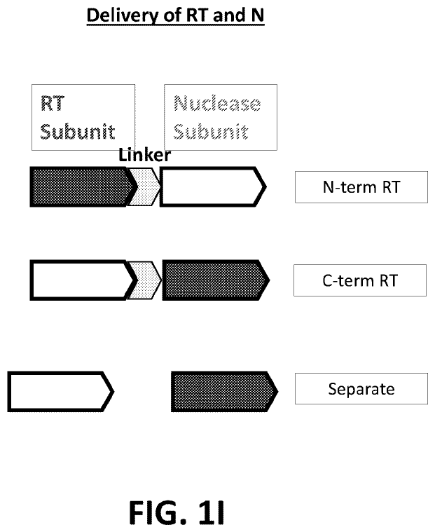

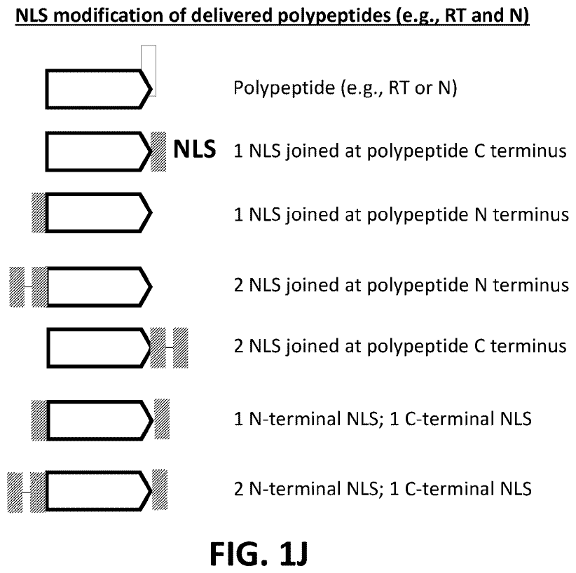

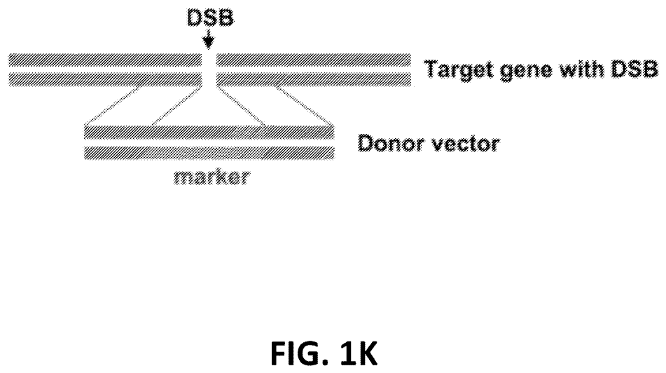

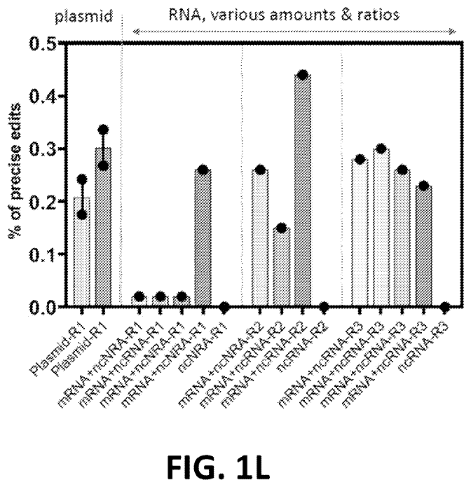

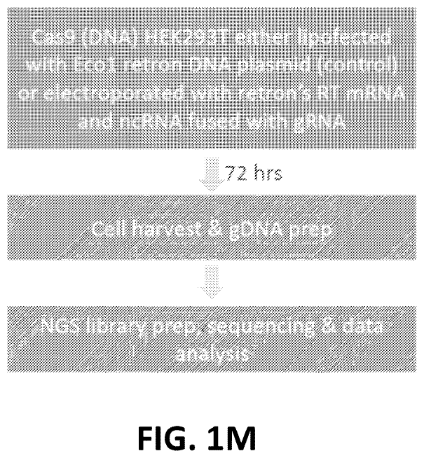

As used herein, the term “retron” refers to a specific type of naturally-occurring and distinct DNA sequence found in the genome of many bacteria which typically encodes three distinct components, namely, (a) a non-coding RNA (“ncRNA”) (comprising contiguous inverted sequences (msr and msd), (b) a reverse transcriptase (RT)-coding gene (ret), and (c) in many cases, a retron-associated gene of unknown function. Retrons are particularly defined by their unique ability to produce a satellite DNA known as msDNA (multicopy single-stranded DNA). The ncRNA (comprising the msr and msd elements) and the ret gene are transcribed as a single polycistronic RNA transcript which processed into the ncRNA transcript and a transcript encoding the ret gene. The ncRNA then becomes folded into a specific secondary structure. Once translated, the RT then binds the folded ncRNA and reverse transcribes the msd region to form a single strand of cDNA (the msDNA) that remains covalently attached to the RNA template via a 2′-5′ phosphodiester bond and base-pairing between the 3′ ends of the msDNA and the RNA template. See FIG. 1A which provides a schematic of the production of an msDNA from a naturally-occurring retron.

### Retron Component

As used herein, the term “retron component” refers to a distinct element or feature of a retron, namely (a) a non-coding RNA (“ncRNA”) (comprising contiguous inverted sequences (msr and msd), (b) a reverse transcriptase (RT)-coding gene (ret), and (c) in many cases, a retron-associated gene of unknown function.

### RNA-Guided Nuclease

As used herein, an “RNA-guided nuclease” is a type of “programmable nuclease,” and a specific type of “nucleic acid-guided nuclease.” As used herein, the term “RNA-guided nuclease” or “RNA-guided endonuclease” refers to a nuclease that associates covalently or non-covalently with a guide RNA thereby forming a complex between the guide RNA and the RNA-guided nuclease. The guide RNA comprises a spacer sequence which comprises a nucleotide sequence having complementarity with a strand of a target DNA sequence. Thus, the RNA-guided nuclease is indirectly guided or programmed to localize to a specific site in a DNA molecule through its association with the guide RNA, which directly binds or anneals to a strand of the target DNA through its complementarity region via Watson-Crick base-pairing.

### Sequence Identity

As used herein, the term “sequence identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). For example, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna. CMP matrix. Methods commonly employed to determine percent identity between sequences include, but are not limited to those disclosed in Carillo, H. and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990).

It is noted that when this disclosure speaks to a polypeptide (including anywhere in this specification, including in Table A and the Examples) having a percent identity with respect to another amino acid sequence (a reference amino acid sequence), such as a polypeptide at least 50%, at least 55%, at least 60%, at least 65%, at least 70%, at least 75%, at least 80%, at least 85%, at least 90%, at least 91%, at least 92%, at least 93%, at least 94%, at least 95, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to another amino acid sequence (a reference amino acid sequence), it is advantageous that in the polypeptide having a percent identity to the reference amino acid sequence conserved regions of the reference amino acid sequence (e.g., conserved when compared with other retron RTs, such as those identified herein) be preserved and/or that the polypeptide has at least one activity selected from reverse transcriptase; endonuclease activity; endoribonuclease activity, or RNA-guided DNase activity and/or that the polypeptide of which comprises: a. one or more α-helical recognition lobe (REC) and a nuclease lobe (NUC); b. a Wedge (WED), α-helical recognition lobe (REC), PAM-interacting (PI), RuvC nuclease, Bridge Helix (BH) and NUC domains; or c. one or more domains selected from RuvC, REC, WED, BH, PI and NUC domains and/or that the polypeptide recognizes or binds a guide RNA or ncRNA as the case may be. Likewise, when this disclosure speaks to a nucleic acid sequence or molecule having a percent identity with respect to a nucleic acid sequence having a percent identity with respect to another nucleic acid sequence or molecule (a reference nucleic acid sequence), such as a nucleic acid sequence at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% identical to another nucleic acid sequence, it is advantageous that in the nucleic acid sequence that has a percent identity to the reference nucleic acid sequence that conserved regions of the reference nucleic acid sequence (e.g., conserved when compared with other retron ncRNAs, such as those identified herein) be preserved and/or that in the polypeptide that is expressed from the nucleic acid sequence that has a percent identity to the reference nucleic acid sequence that the polypeptide contain conserved region(s) (e.g., conserved when compared with other retron sequences, such as those identified herein) and/or that the polypeptide has at least one activity selected from reverse transcriptase; endonuclease activity; endoribonuclease activity, or RNA-guided DNase activity and/or that the polypeptide of which comprises: a. one or more α-helical recognition lobe (REC) and a nuclease lobe (NUC); b. a Wedge (WED), α-helical recognition lobe (REC), PAM-interacting (PI), RuvC nuclease, Bridge Helix (BH) and NUC domains; or c. one or more domains selected from RuvC, REC, WED, BH, PI and NUC domains and/or that the polypeptide recognizes or binds a guide RNA.

### Subject

As used herein, the term“subject” refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. The terms “individual,” “subject,” “host,” and “patient,” used interchangeably herein.

### Stem

As used herein, the term “stem” refers to two or more base pairs, such as 3, 4, 5, 6, 7, 8, 9, 10, 11, 12, 13, 14, 15, 16, 17, 18, 19, 20 or more base pairs, formed by inverted repeat sequences connected at a “tip,” where the more 5′ or “upstream” strand of the stem bends to allows the more 3′ or “downstream” strand to base-pair with the upstream strand. The number of base pairs in a stem is the “length” of the stem. The tip of the stem is typically at least 3 nucleotides, but can be 4, 5, 6, 7, 8, 9, 10, 11, 12, 13, 14, 15 or more nucleotides. Larger tips with more than 5 nucleotides are also referred to as a “loop.” An otherwise continuous stem may be interrupted by one or more bulges as defined herein. The number of unpaired nucleotides in the bulge(s) are not included in the length of the stem. The position of a bulge closest to the tip can be described by the number of base pairs between the bulge and the tip (e.g., the bulge is 4 bps from the tip). The position of the other bulges (if any) further away from the tip can be described by the number of base pairs in the stem between the bulge in question and the tip, excluding any unpaired bases of other bulges in between.

### Synthetic or Artificial Nucleic Acid

A “synthetic or artificial nucleic acid” refers nucleic acids that are non-naturally occurring sequences. Such sequences do not originate from, or are not known to be present in any living organism (e.g., based on sequence search in existing sequence databases). Recombinant nucleic acids and synthetic nucleic acids also include those molecules that result from the replication of either of the foregoing. Engineered nucleic acid constructs of the present disclosure, such as the engineer ed retron described herein, may be encoded by a single molecule (e.g., encoded by or present on the same plasmid or other suitable vector) or by multiple different molecules (e.g., multiple independently-replicating vectors).

### Target Site

As used herein, a “target site” as used herein is a polynucleotide (e.g., DNA such as genomic DNA) that includes a site or specific locus (“target site” or “target sequence”) targeted by a recombinant retron genome modification system disclosed herein. In the context of retron genome modification systems disclosed herein that comprise an RNA-guided nuclease, a target sequence is the sequence to which the guide sequence of a guide nucleic acid (e.g., guide RNA) will hybridize. For example, the target site (or target sequence) 5′-GTCAATGGACC-3′ (SEQ ID NO:19933) within a target nucleic acid is targeted by (or is bound by, or hybridizes with, or is complementary to) the sequence 5′-GGTCCATTGAC-3′ (SEQ ID NO:19934). Suitable hybridization conditions include physiological conditions normally present in a cell. For a double stranded target nucleic acid, the strand of the target nucleic acid that is complementary to and hybridizes with the guide RNA is referred to as the “complementary strand” or “target strand”; while the strand of the target nucleic acid that is complementary to the “target strand” (and is therefore not complementary to the guide RNA) is referred to as the “non-target strand” or “non-complementary strand.”

### Treatment

As used herein, the terms“treatment,” “treat,” and“treating,” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Upstream and Downstream

As used herein, the terms “upstream” and “downstream” are terms of relativity that define the linear position of at least two elements located in a nucleic acid molecule (whether single or double-stranded) that is orientated in a 5′-to-3′ direction. A first element is said to be upstream of a second element in a nucleic acid molecule where the first element is positioned somewhere that is 5′ to the second element. Conversely, a first element is downstream of a second element in a nucleic acid molecule where the first element is positioned somewhere that is 3′ to the second element.

### Variant

As used herein the term“variant” should be taken to mean the exhibition of qualities that have a pattern that deviates from what occurs in nature, e.g., a variant retron RT is retron RT comprising one or more changes in amino acid residues as compared to a wild type retron RT amino acid sequence. The term“variant” encompasses homologous proteins having at least 75%, or at least 80%, or at least 85%, or at least 90%, or at least 95%, or at least 99% percent identity with a reference sequence and having the same or substantially the same functional activity or activities as the reference sequence. The term also encompasses mutants, truncations, or domains of a reference sequence, and which display the same or substantially the same functional activity or activities as the reference sequence.

### Vector

As used herein, the term “vector” permits or facilitates the transfer of a polynucleotide from one environment to another. It is a replicon such as a plasmid, phage, or cosmid into which another DNA segment may be inserted so as to bring about the replication of the inserted segment (e.g., the subject engineered retron). Generally, a vector is capable of replication when associated with the proper control elements. The term “vector” may include cloning and expression vectors, as well as viral vectors and integrating vectors.

### Wild Type

As used herein the term “wild type” is a term of the art understood by skilled persons and means the typical form of an organism, strain, gene, protein, or characteristic as it occurs in nature as distinguished from mutant or variant forms.

### DETAILED DESCRIPTION

The present disclosure provides systems, methods and compositions used for precise genome editing, including installing nucleic acid insertions, replacements, and deletions at targeted and precise genome sites, wherein said systems, methods, and compositions are based on novel and/or modified retrons or components thereof, such as modified versions of the retron RTs of Table X, modified versions of the ncRNAs of Table A, and modified versions of the RTs of Table B.

In one aspect, the present disclosure provides recombinant retrons comprising one or more genetic modifications which improves the functionality and/or properties of a retron. Such genetic modifications can include a mutation, insertion, deletion, inversion, replacement, substitution, or translocation of one or more contiguous or non-contiguous nucleobases in a nucleic acid molecule encoding a retron or a component of a retron, such as an ncRNA or a reverse transcriptase. In various aspects, the retron that becomes modified with the one or more genetic modifications (i.e., the “pre-modified” or “unmodified” retron or retron component) is a naturally occurring retron or retron component (e.g., naturally occurring ncRNA of Table A or RT) ability to facilitate homology-dependent recombination (or HDR) in a cell, thereby resulting in a relative increase in the concentrations or amounts of msDNA comprising a DNA donor template. In particular embodiments, the recombinant retrons are based on and/or derived from a naturally-occurring retron, such as any retron-related sequence provided by Table X (the introduction of the one or more genetic modifications into a set of 7257 previously unknown retrons discovered through computational methods described herein (e.g., see Examples). In other embodiments, the recombinant retrons are based on introducing the one or more genetic modifications into previously available retron sequences (e.g., the “Mestre et al., Systematic Prediction of Genes Functionally Associated with Bacterial Retrons and Classification of The Encoded Tripartite Systems, Nucleic Acids Research, Volume 48, Issue 22, 16 Dec. 2020, Pages 12632-12647” (incorporated herein by reference) to achieve recombinant retrons with the enhanced ability to produce increased concentrations or amounts of msDNA comprising a DNA donor template.

In another aspect, the present disclosure further provides nucleic acid molecules encoding the recombinant retrons and/or recombinant retron components (e.g., a recombinant ncRNA and/or a recombinant retron RT). In still another aspect, the present disclosure provides genome editing systems comprising recombinant retron components (e.g., recombinant ncRNA and/or recombinant RT), programmable nucleases (e.g., RNA-guided nucleases, such as CRISPR-Cas proteins, ZFPs, and TALENS), and guide RNAs (in the case where RNA-guide nucleases are used in said genome editing systems). In a further aspect, the disclosure provides nucleic acid molecules encoding the described genome editing systems and said components thereof, as well as polypeptides making up the components of said genome editing systems. In yet another aspect, the disclosure provides vectors for transferring and/or expressing said genome editing systems, e.g., under in vitro, ex vivo, and in vivo conditions. In still another aspect, the disclosure provides cell-delivery compositions and methods, including compositions for passive and/or active transport to cells (e.g., plasmids), delivery by virus-based recombinant vectors (e.g., AAV and/or lentivirus vectors), delivery by non-virus-based systems (e.g., liposomes and LNPs), and delivery by virus-like particles. Depending on the delivery system employed, the retron-based genome editing systems described herein may be delivered in the form of DNA (e.g., plasmids or DNA-based virus vectors), RNA (e.g., ncRNA and mRNA delivered by LNPs), a mixture of DNA and RNA, protein (e.g., virus-like particles), and ribonucleoprotein (RNP) complexes. Any suitable combinations of approaches for delivering the components of the herein disclosed retron-based genome editing systems may be employed. In one embodiment, each of the components of the retron-based genome editing system is delivered by an all-RNA system, e.g., the delivery of one or more RNA molecules (e.g., mRNA and/or ncRNA) by one or more LNPs, wherein the one or more RNA molecules form the ncRNA and guide RNA (as needed) and/or are translated into the polypeptide components (e.g., the RT and a programmable nuclease). In yet another aspect, the disclosure provides methods for genome editing by introducing a retron-based genome editing system described herein into a cell (e.g., under in vitro, in vivo, or ex vivo conditions) comprising a target edit site, thereby resulting in an edit at the target edit. In other aspects, the disclosure provides formulations comprising any of the aforementioned components for delivery to cells and/or tissues, including in vitro, in vivo, and ex vivo delivery, recombinant cells and/or tissues modified by the recombinant retron-based genome modification systems and methods described herein, and methods of modifying cells by conducting genome editing and related DNA donor-dependent methods, such as recombineering, or cell recording, using the herein disclosed retron-based genome modification systems. The disclosure also provides methods of making the recombinant retrons, retron-based genome modification systems, vectors, compositions and formulations described herein, as well as to pharmaceutical compositions and kits for modifying cells under in vitro, in vivo, and ex vivo conditions that comprise the herein disclosed genome editing and/or modification systems.

Described herein are engineered retrons comprising one or more heterologous nucleic acids. The one or more heterologous nucleic acids may be inserted, for example, at or within a location selected from: the msd locus, upstream of the msr locus, upstream of the msd locus, and downstream of the msd locus. In some embodiments, the engineered retrons have structural improvements over their naturally existing counterparts or wild-type retrons at least with respect to the encoded ncRNA and/or the reverse transcriptase (RT), such that the engineered retron or the encoded ncRNA thereof, when delivered to a host cell, such as a mammalian host cell, exhibits various functional improvements over its naturally existing/wild-type retron elements.

Exemplary (non-limiting functional improvements) may include any one or more of the features described herein. For example, in some embodiments, the engineered retron may comprise a sequence modification (e.g., insertion, deletion, and/or substitution of one or more nucleotide(s)) in the msr locus and/or the msd locus that: i) modulates (e.g., enhances) reverse transcription, processivity, accuracy/fidelity, and/or production of the msDNA (e.g., in the mammalian cell); ii) modulates (e.g., reduces) immunogenicity of the ncRNA encoded by the engineered retron (e.g., encoded by the msr locus and/or the msd locus) in a host (e.g., a host comprising the mammalian cell); iii) comprises a nucleotide sequence that modulates (e.g., inhibits or antagonizes) a function of the msDNA; and/or iv) modulates (e.g., improves) efficiency of targeted genomic engineering.

Thus, in general, the engineered retron is an engineered nucleic acid construct comprising: a) a first polynucleotide encoding a non-coding RNA (ncRNA), said first polynucleotide comprising: i) an msr locus encoding the msr RNA portion of a multi-copy single-stranded DNA (msDNA); and ii) an msd locus encoding the msd RNA portion of the msDNA; and

b) one or more heterologous nucleic acids inserted at or within a location selected from: the msd locus, upstream of the msr locus, upstream of the msd locus, and downstream of the msd locus.

The engineered nucleic acid construct (e.g., the engineered retron) may further comprise a second polynucleotide encoding a reverse transcriptase (RT), or a portion thereof, wherein the encoded RT is capable of synthesizing a DNA copy of at least a portion of the msd locus encoding the msDNA.

In certain embodiments, the engineered retron of the invention encodes a reverse transcriptase (RT) or a functional domain thereof, comprising: i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or ii) a polypeptide listed in any one of Table C. In some embodiments, the RT does not comprise a polypeptide listed in Table X.

In certain embodiments, the engineered retron of the invention encodes a reverse transcriptase (RT) or a functional domain thereof, comprising: i) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide listed in Table A; and/or ii) a consensus polynucleotide sequence listed in Table C. In some embodiments, the polynucleotide encoding the RT does not comprise a polynucleotide of Table X.

In certain embodiments, the engineered retron of the invention encodes an ncRNA comprising: (I) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA in Table B.

In certain embodiments, the engineered retron of the invention encodes an ncRNA and a reverse transcriptase (RT) or a functional domain thereof, wherein the ncRNA and the RT or functional domain thereof are as described above.

Specifically, in such embodiment, the ncRNA may comprise: (I) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B.

Also in such embodiment, the reverse transcriptase (RT) or functional domain thereof comprises: (A) i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or ii) a polypeptide listed in Table C; optionally, the RT does not comprise a polypeptide listed in Table X; OR (B) i) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.10%, at least 99.2%, at least 99.3%, at least 99.4%, at least 99.5%, at least 99.6%, at least 99.7%, at least 99.8% or at least 99.9% sequence identity to a polynucleotide in Table A; and/or optionally, the polynucleotide encoding the RT does not comprise a polynucleotide in Table X.

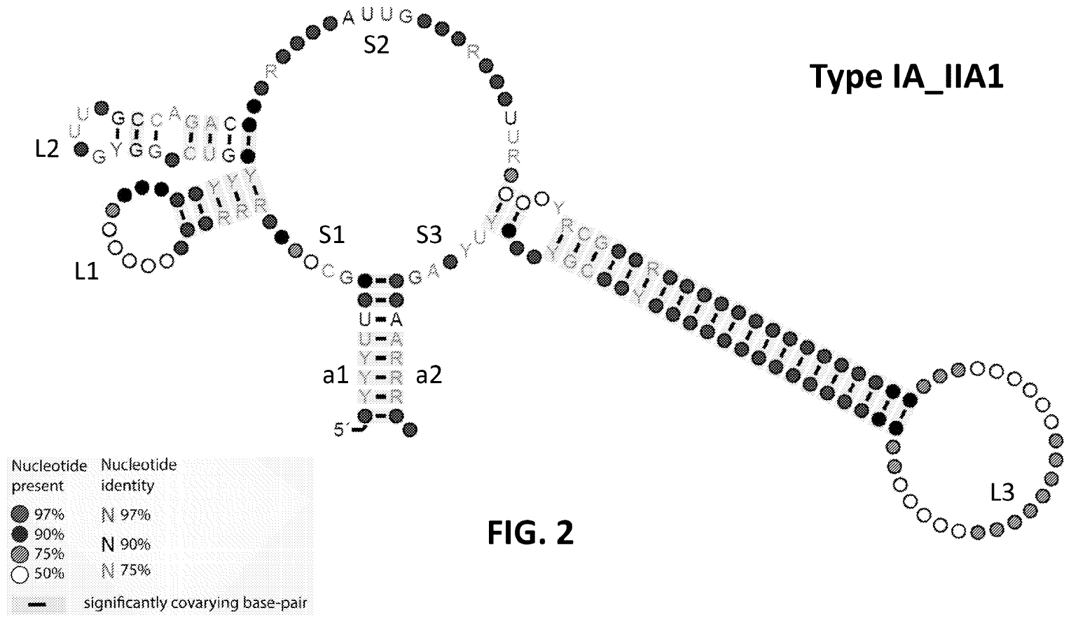

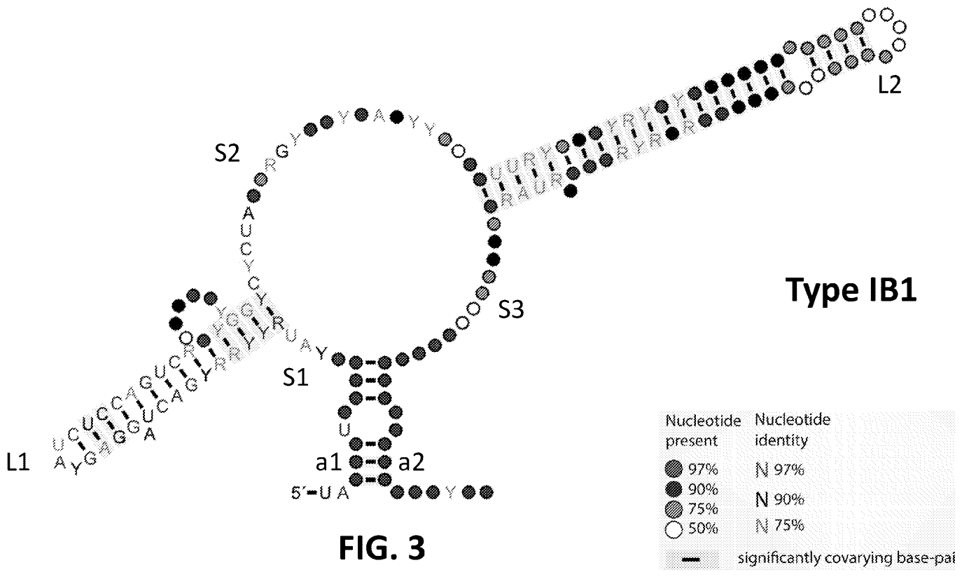

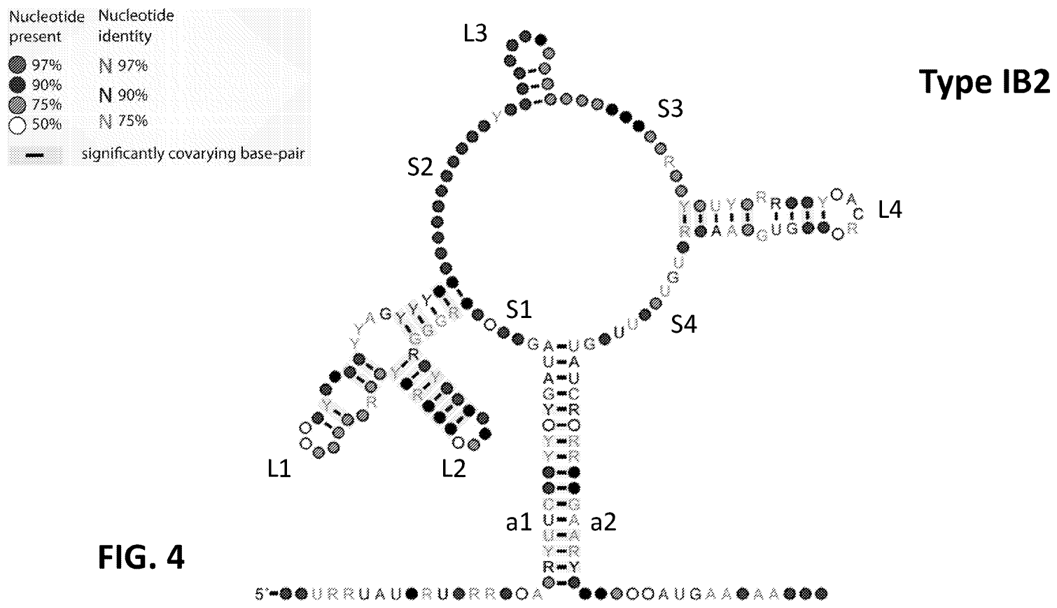

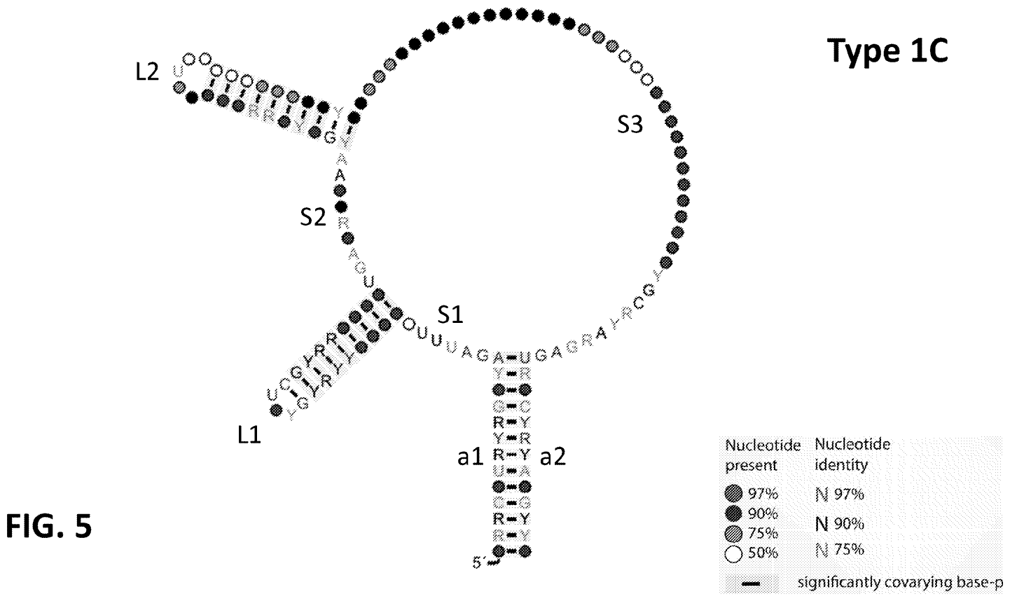

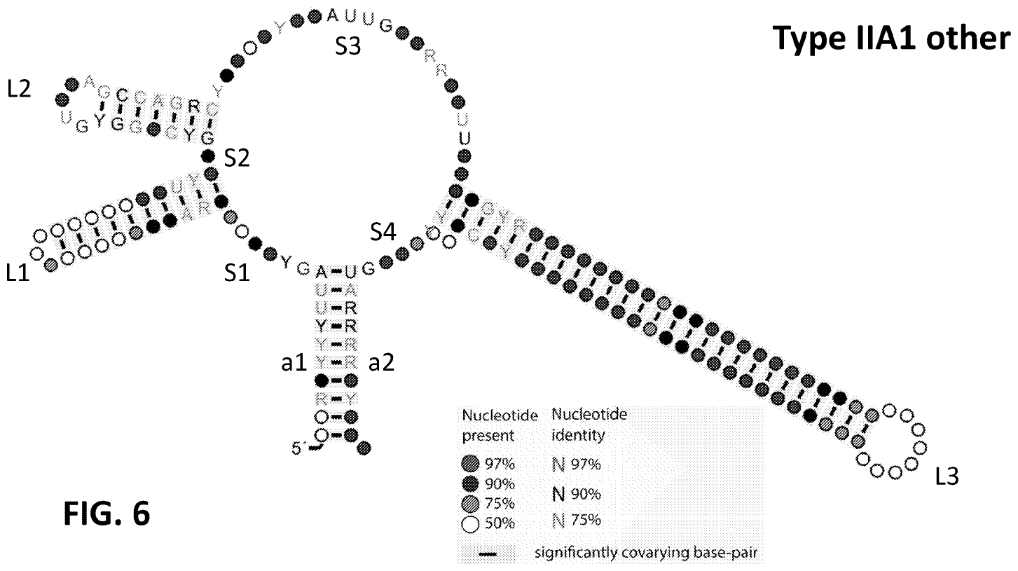

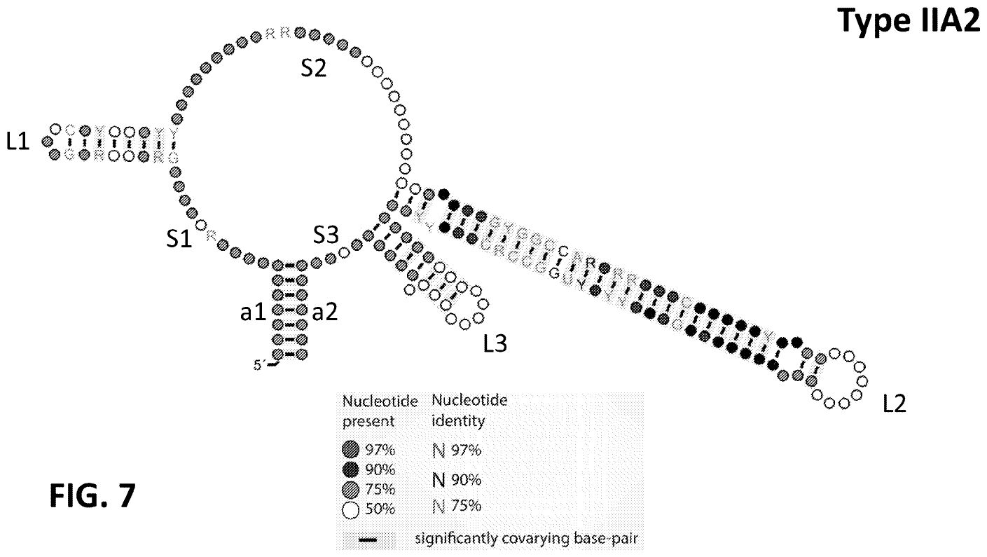

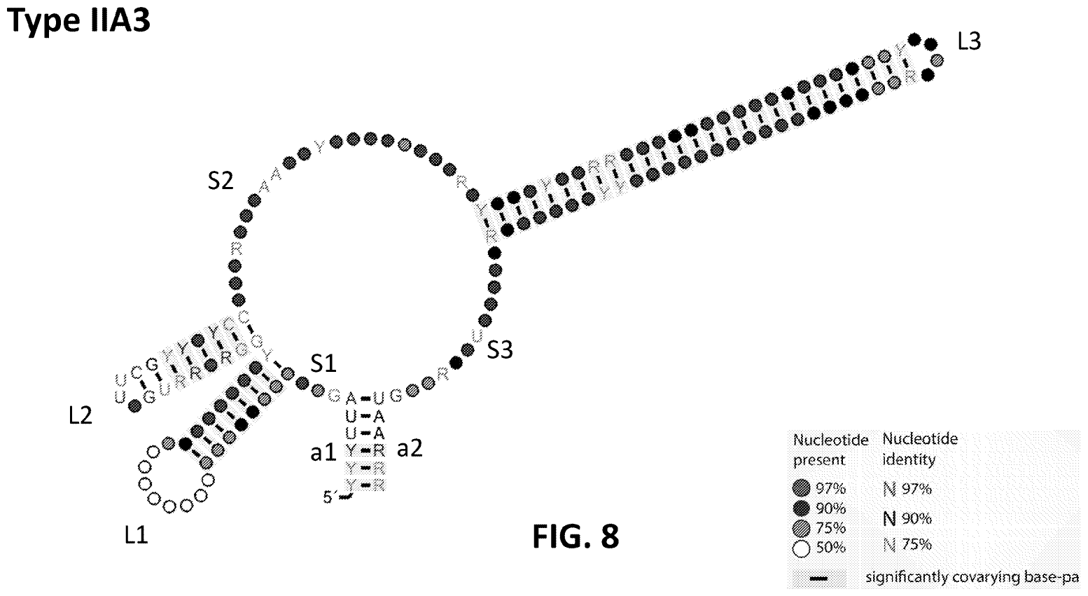

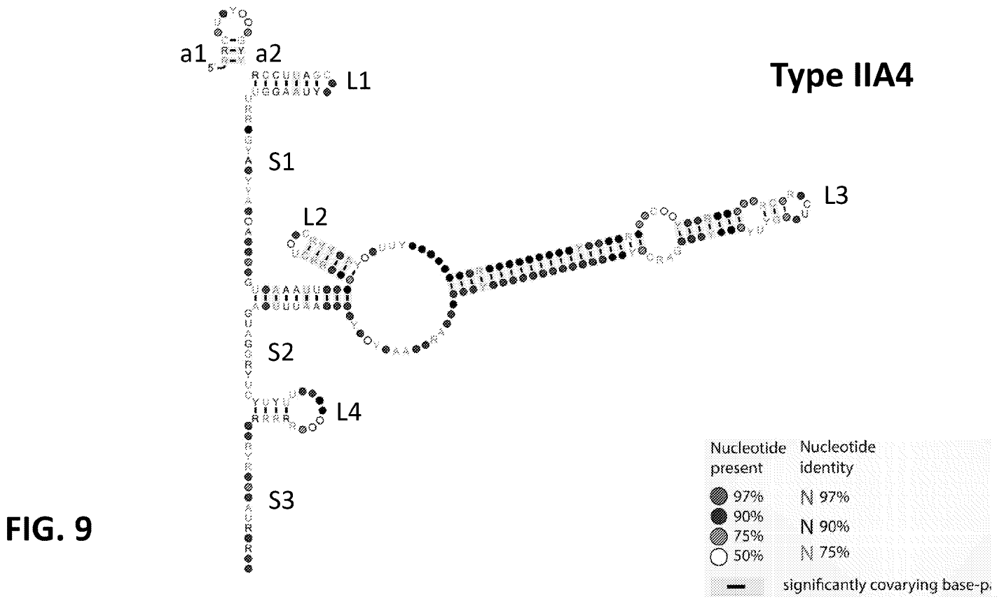

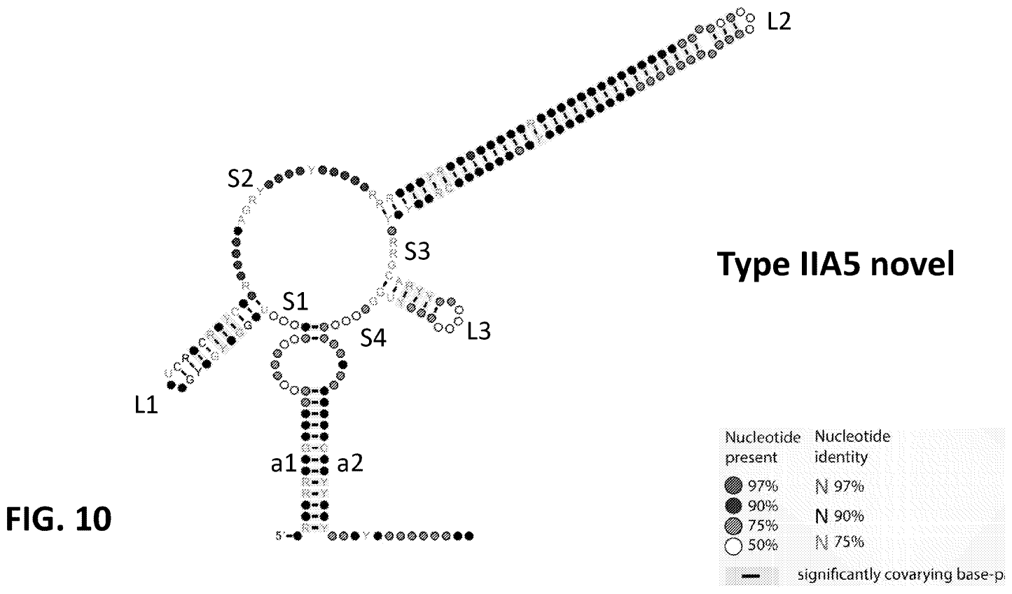

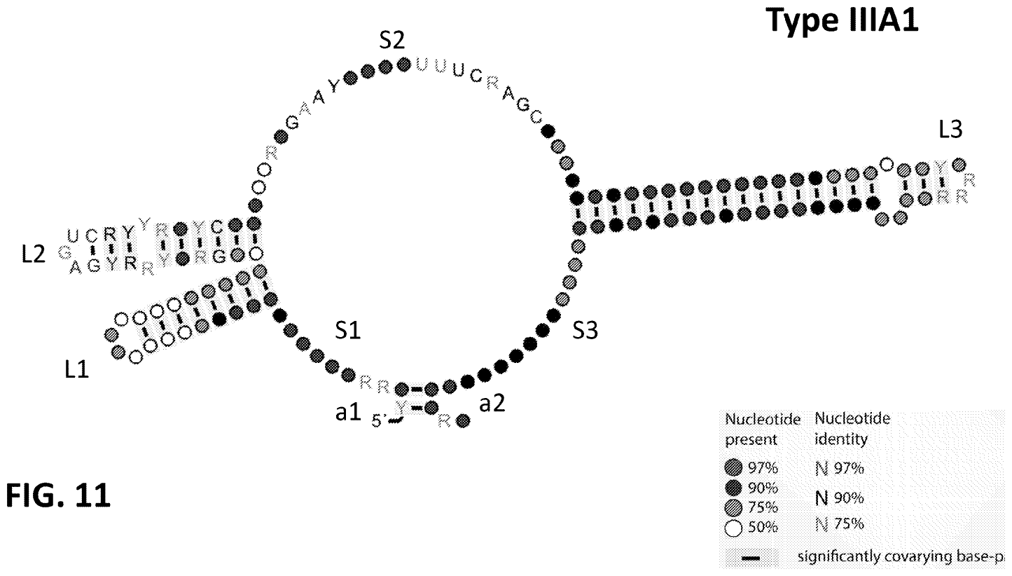

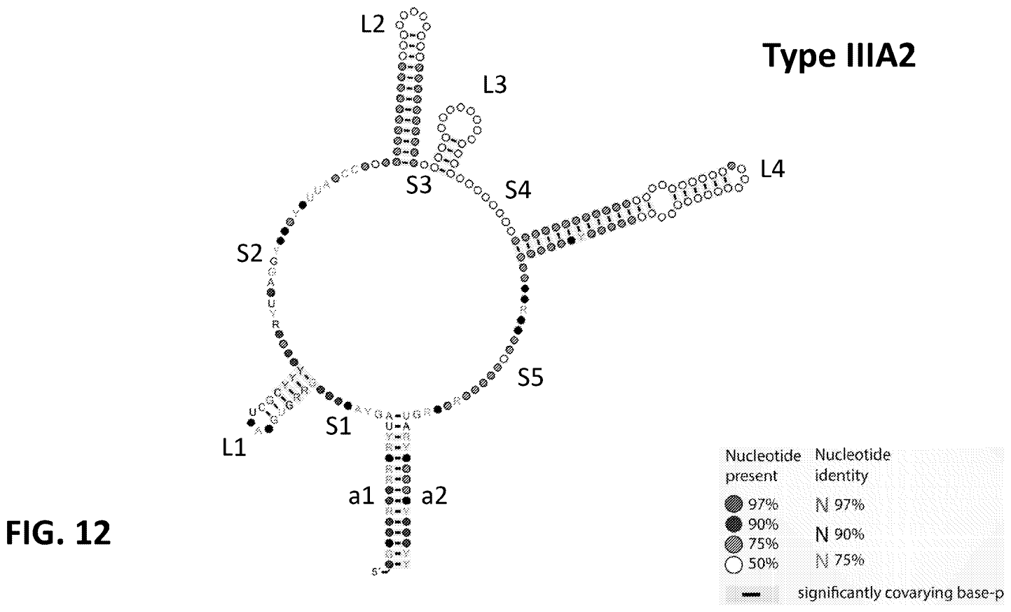

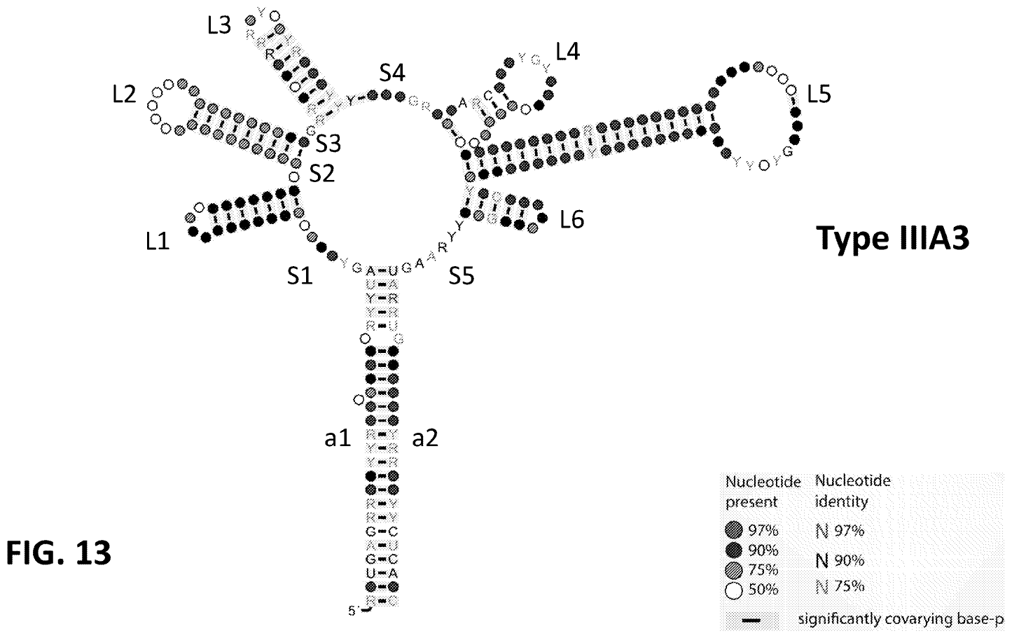

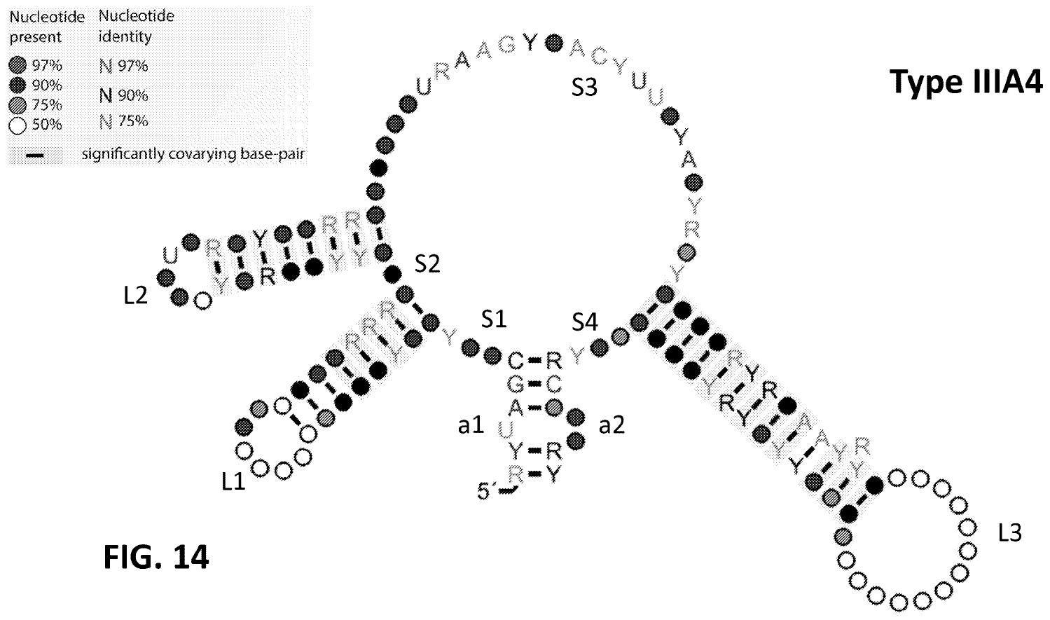

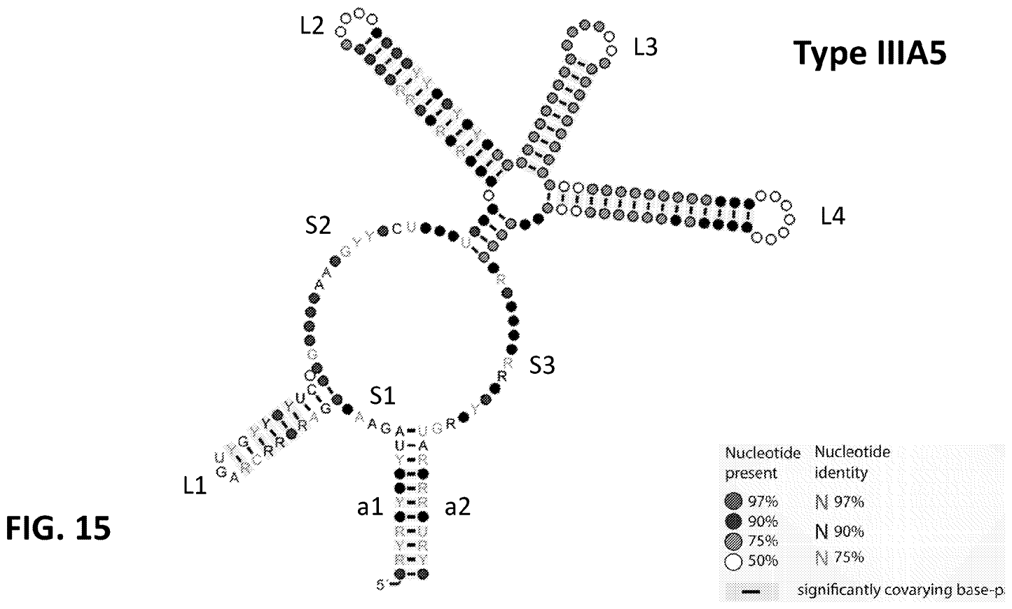

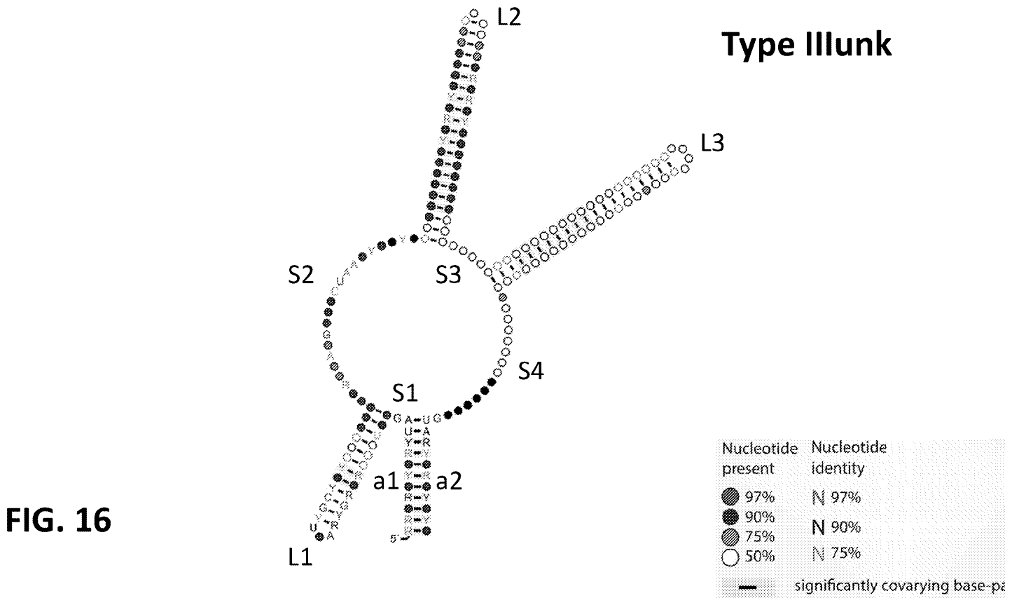

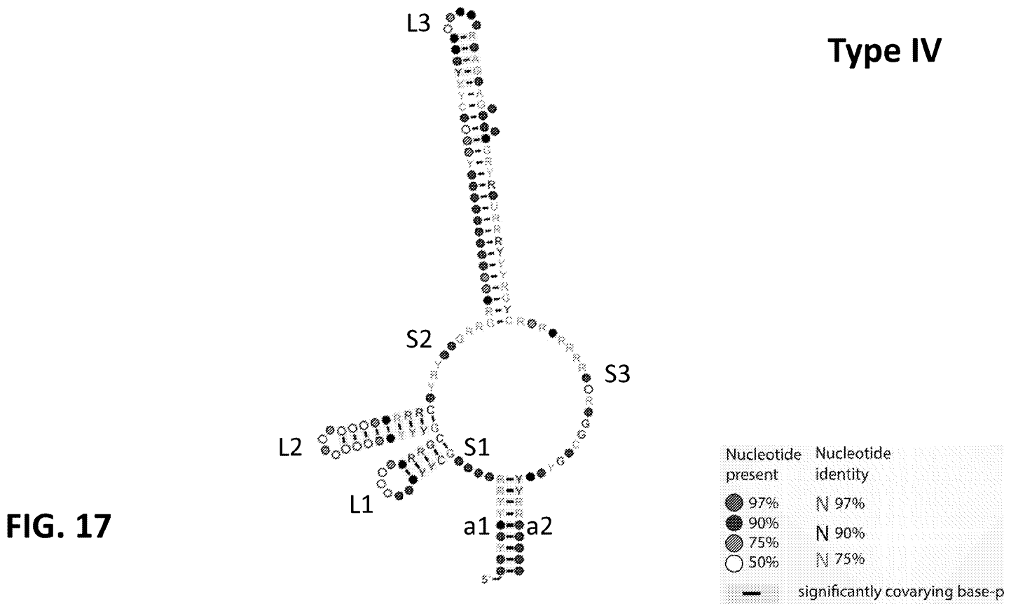

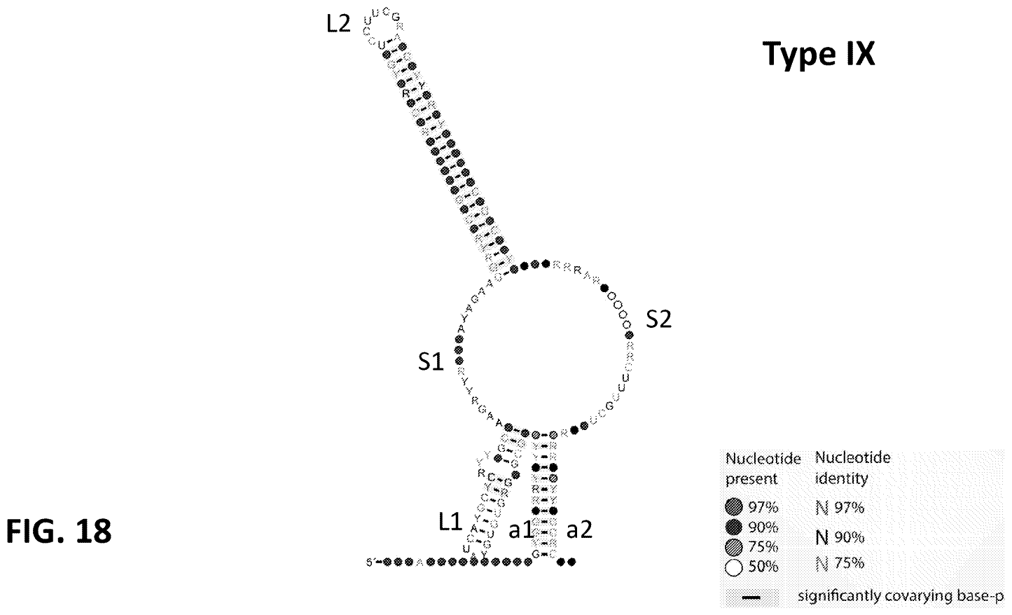

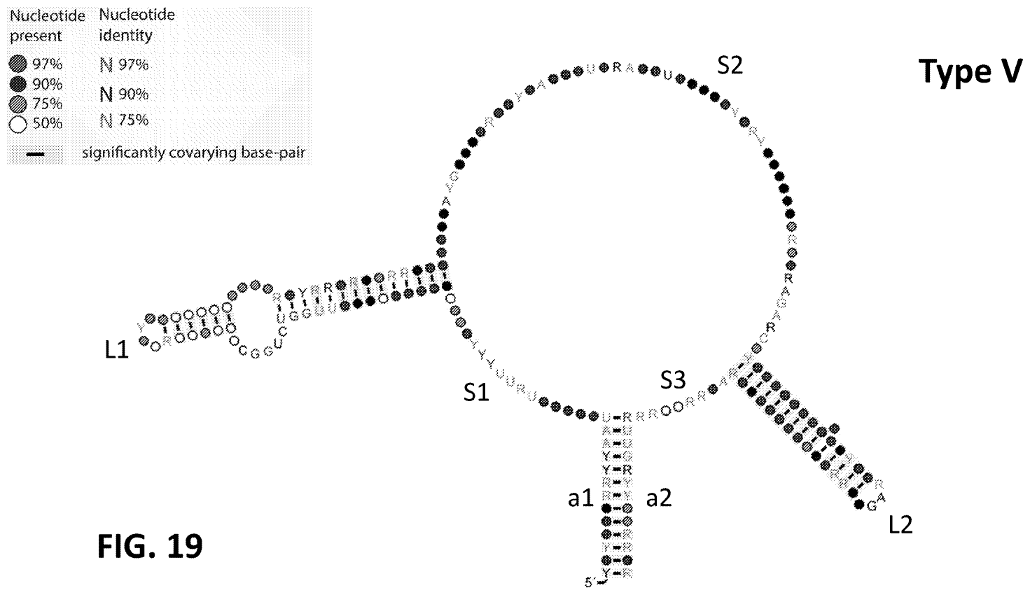

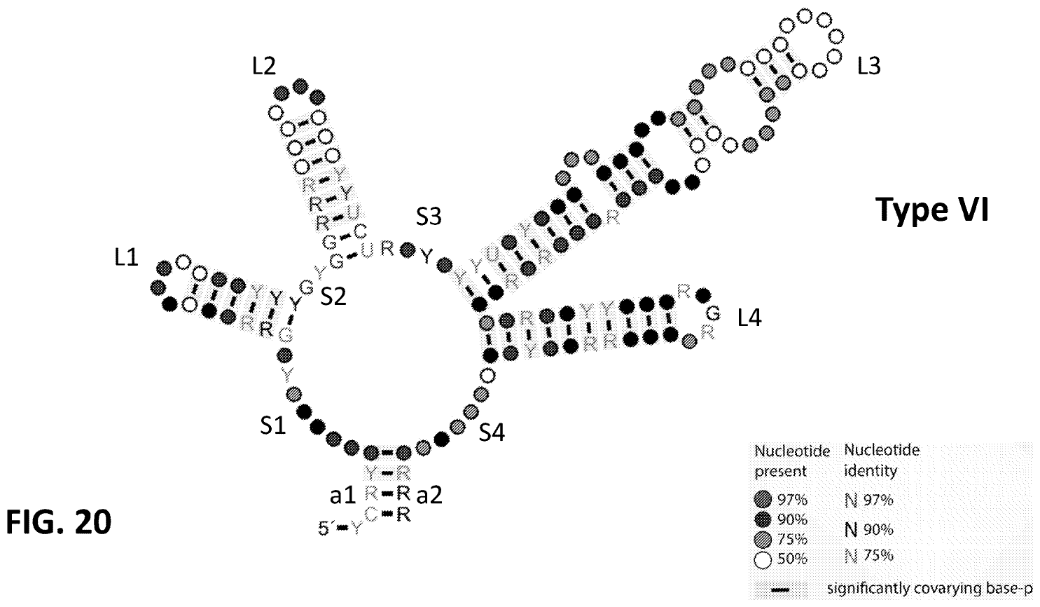

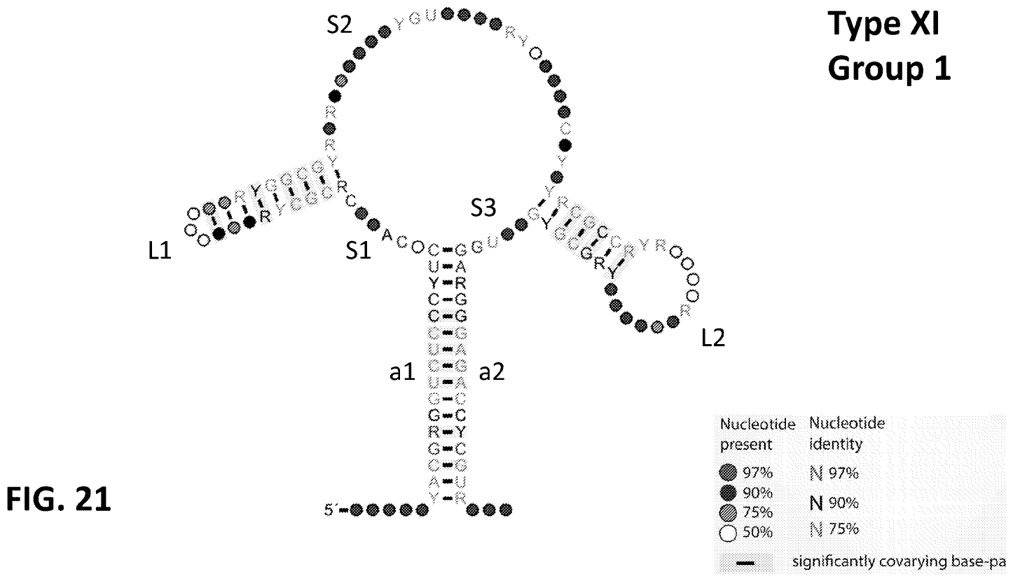

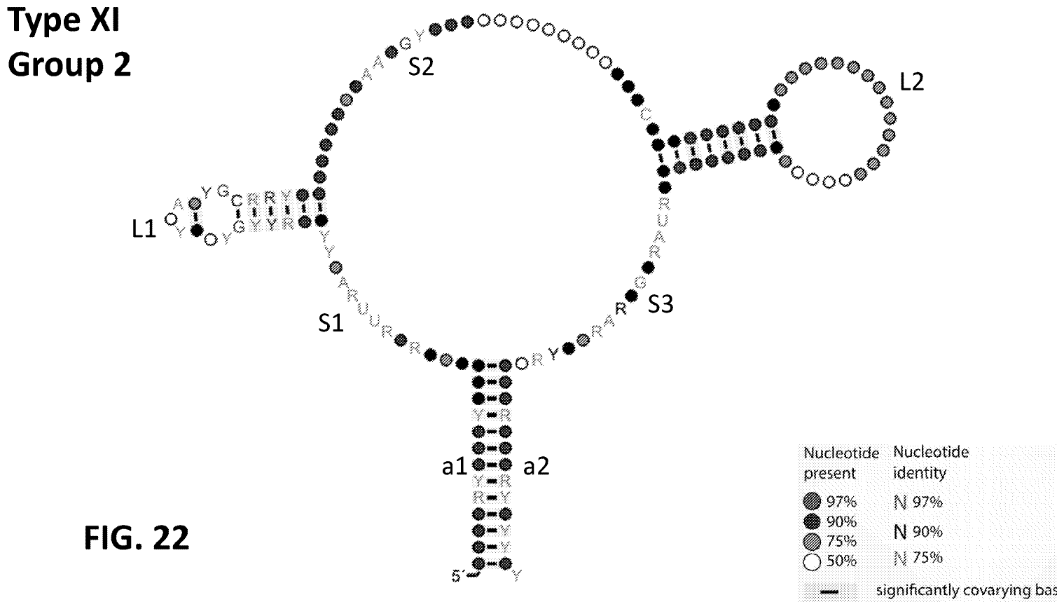

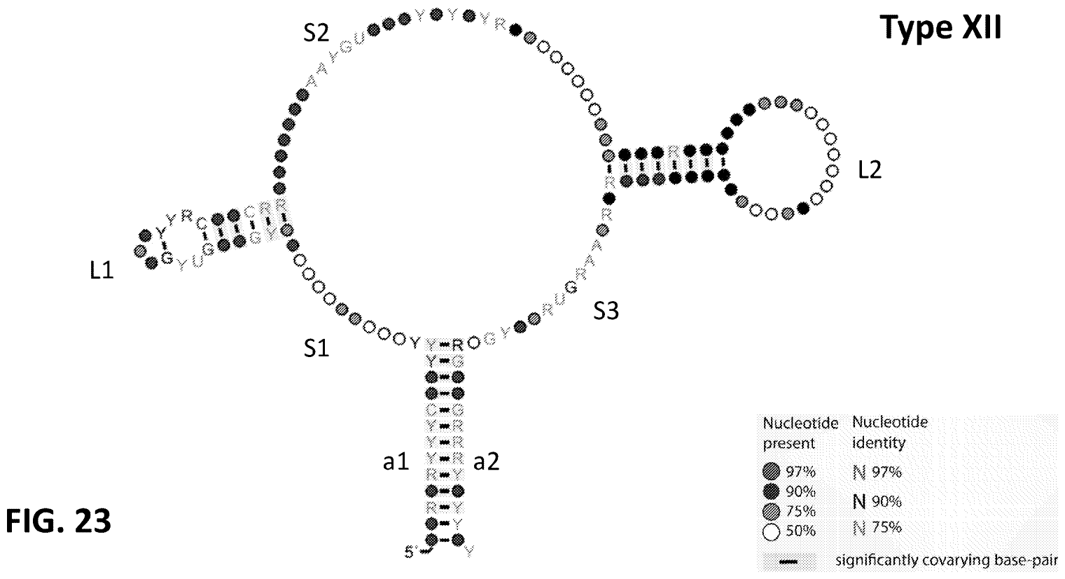

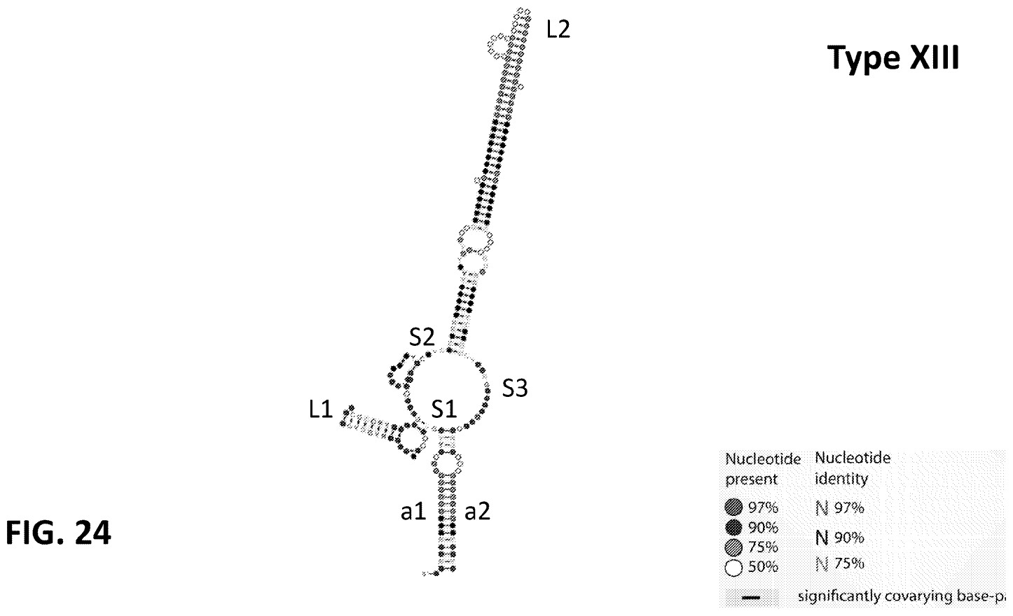

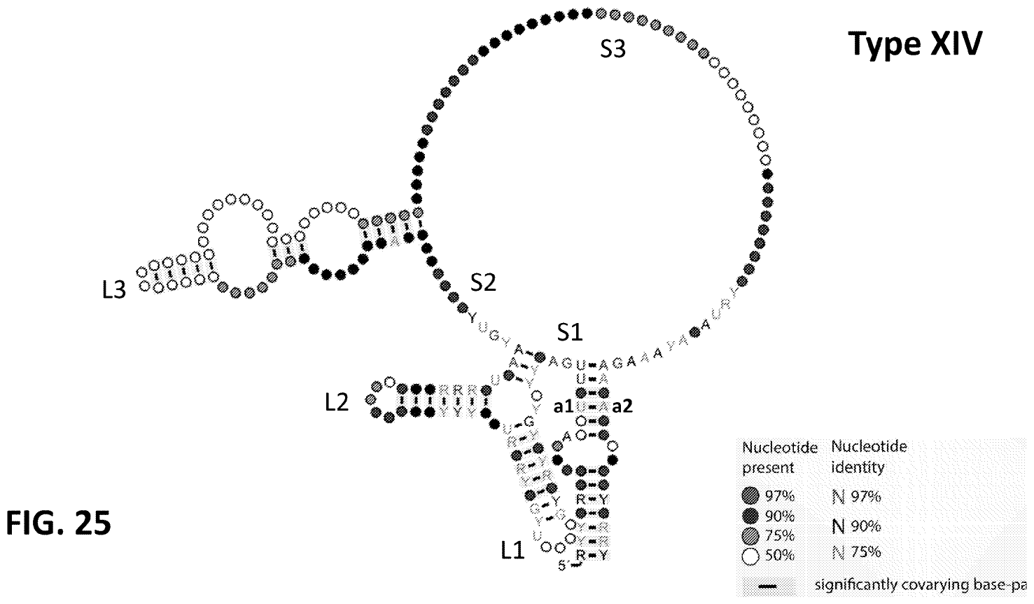

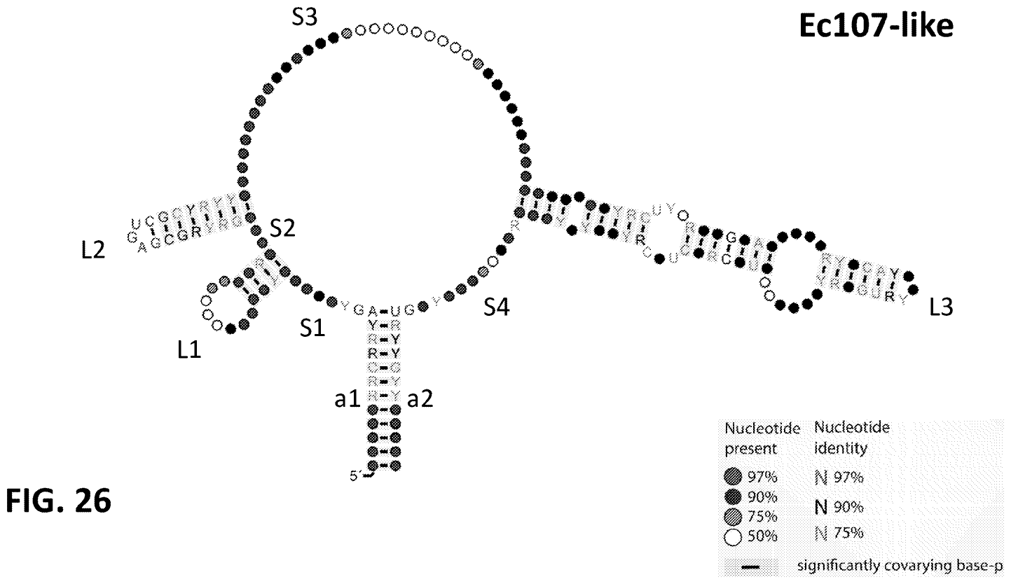

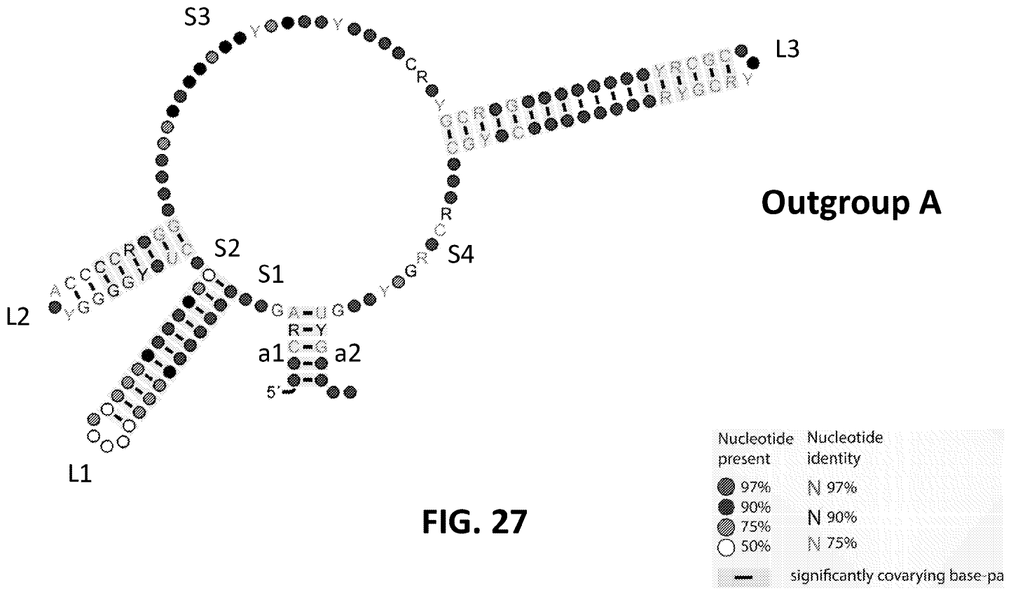

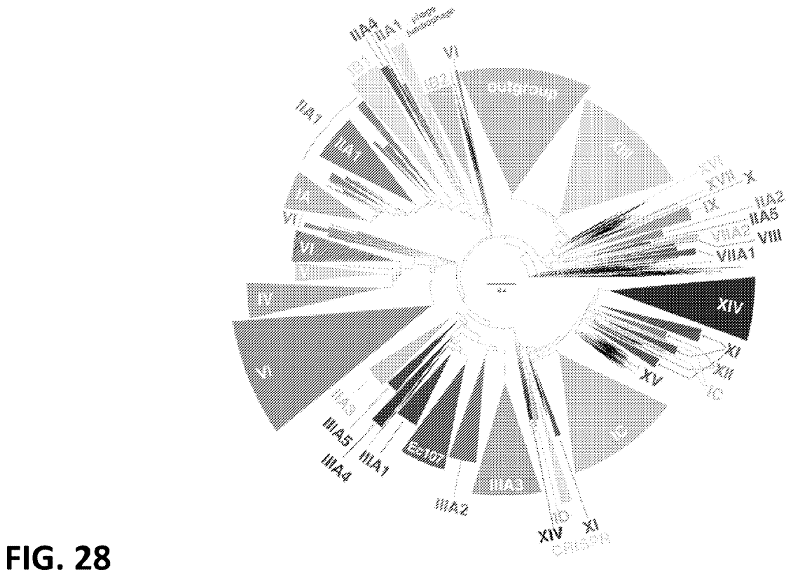

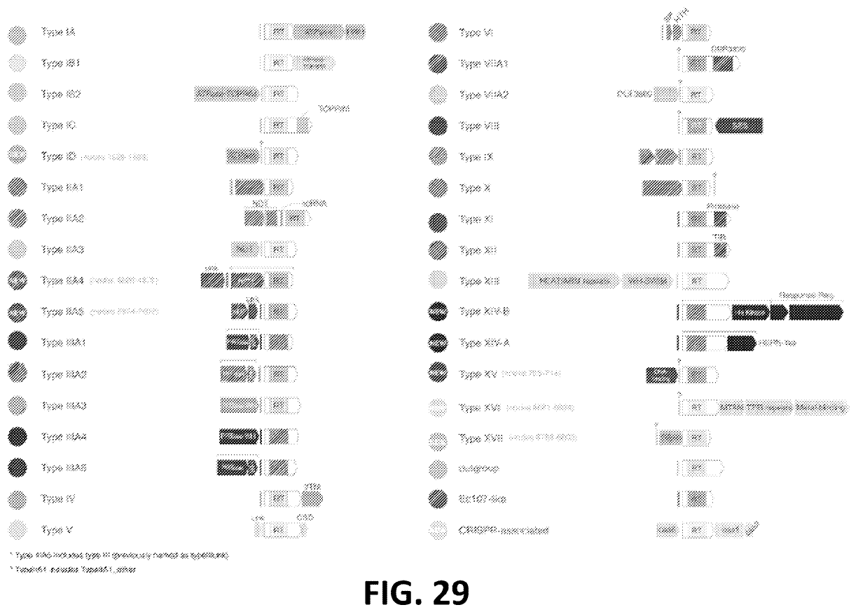

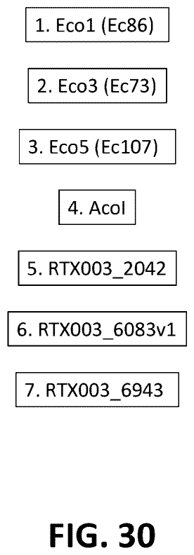

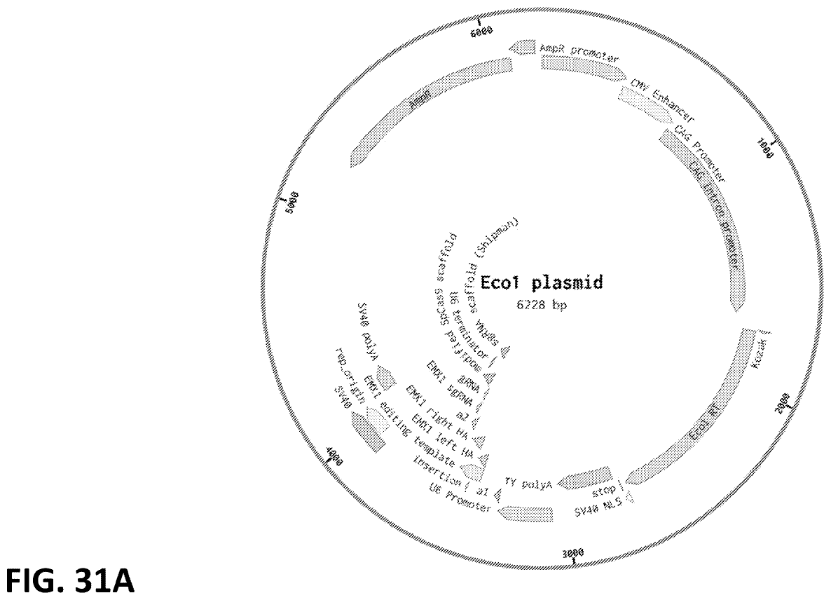

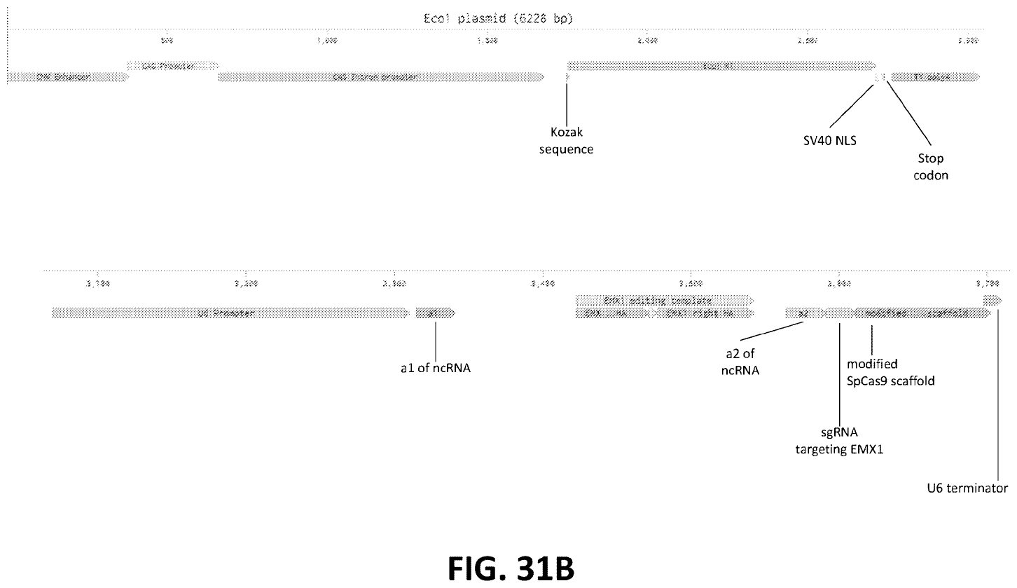

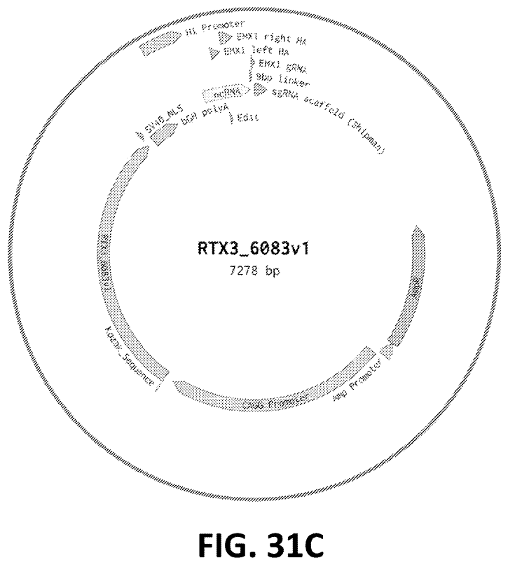

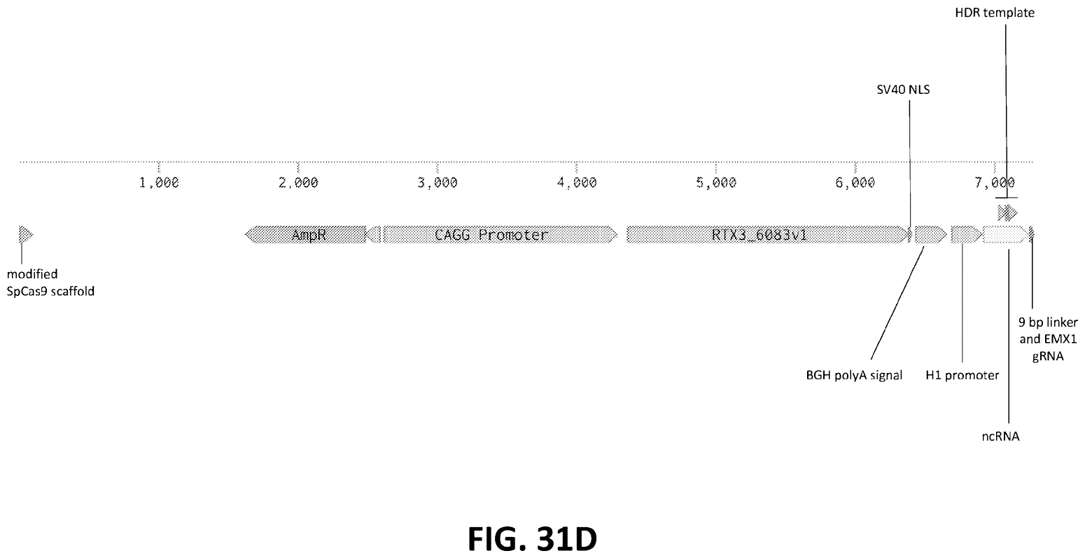

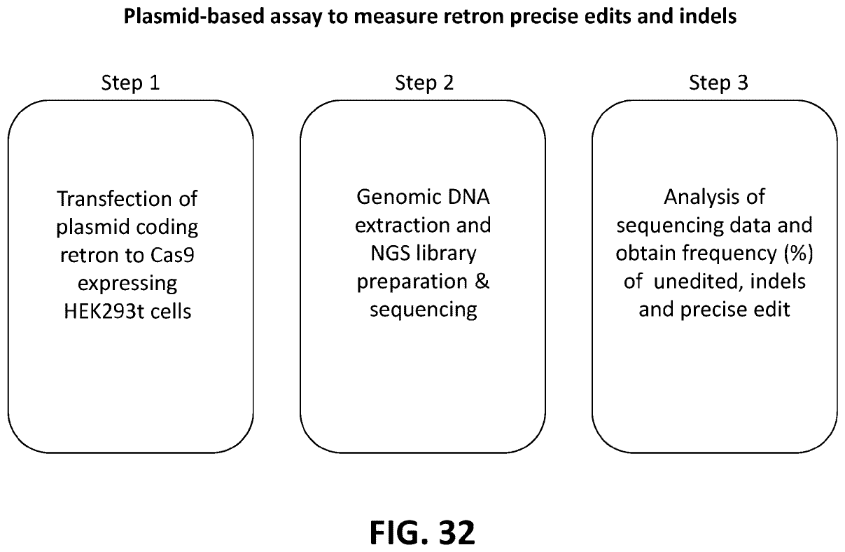

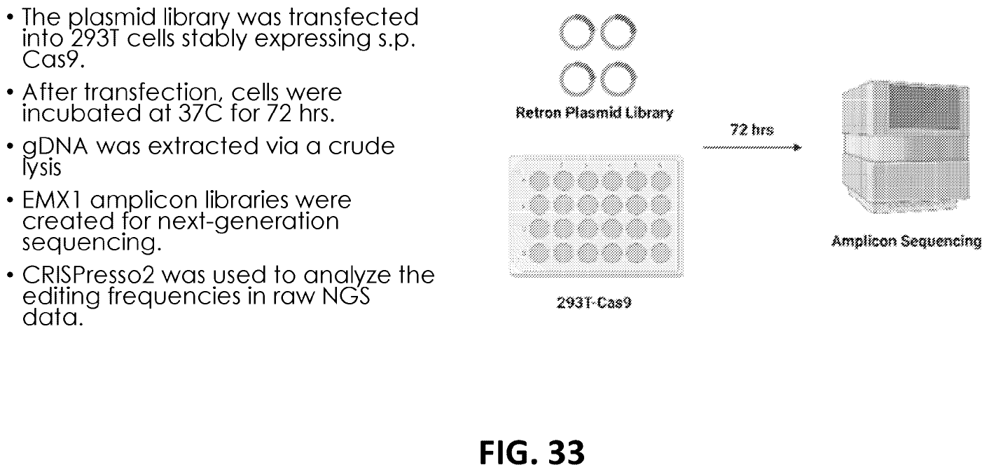

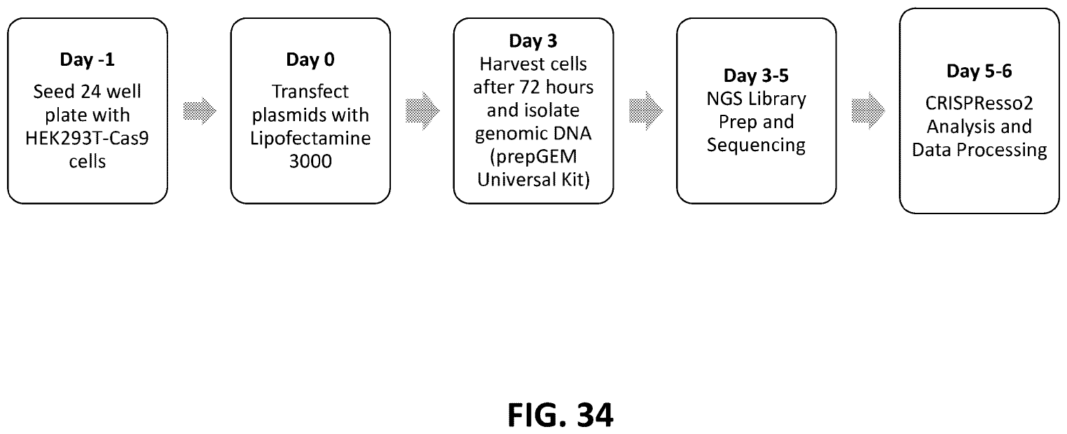

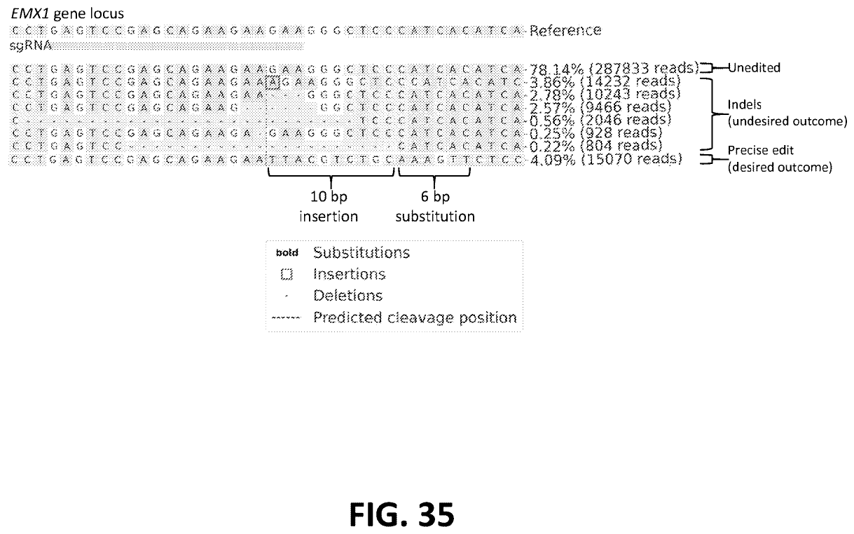

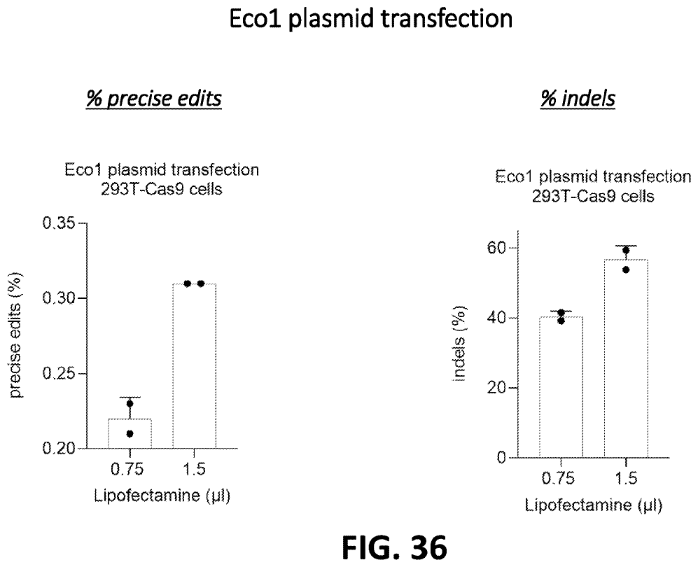

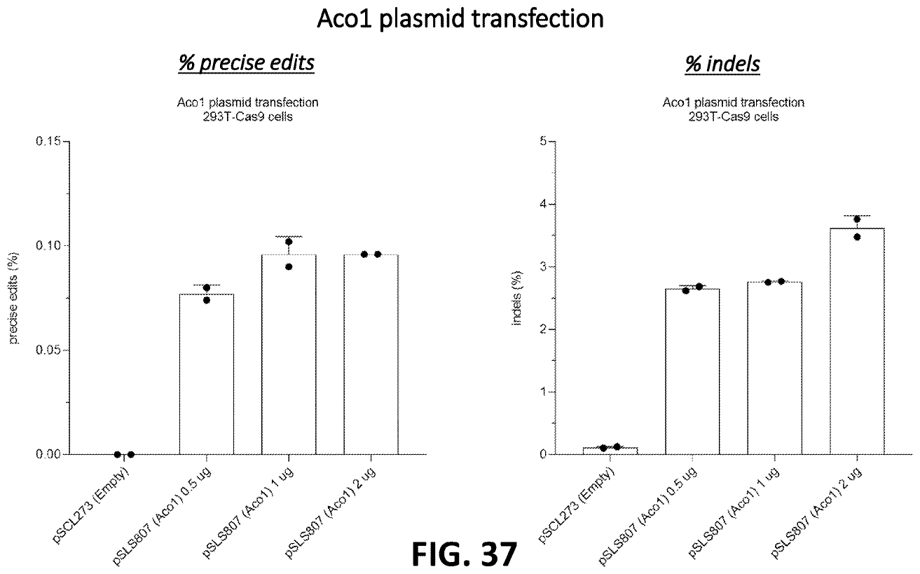

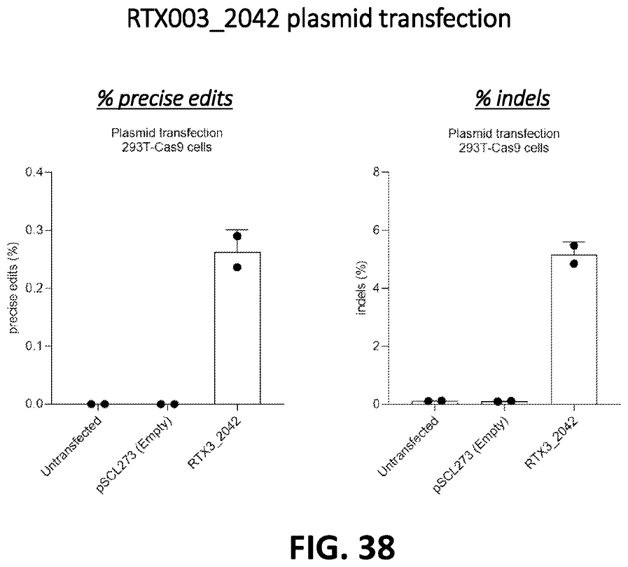

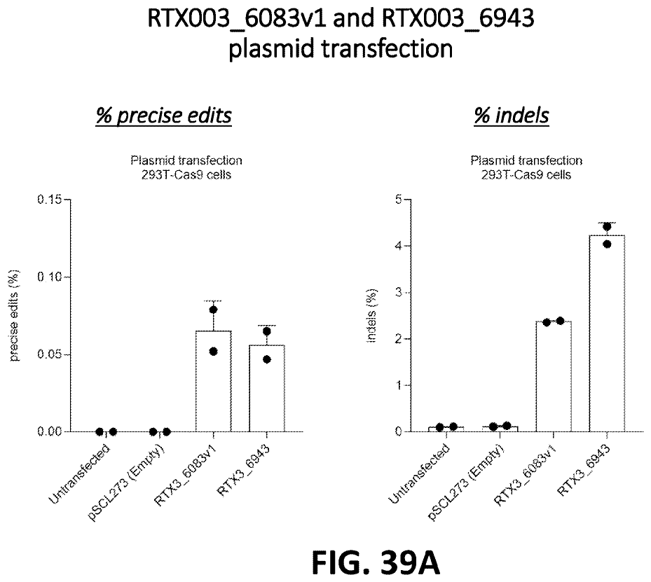

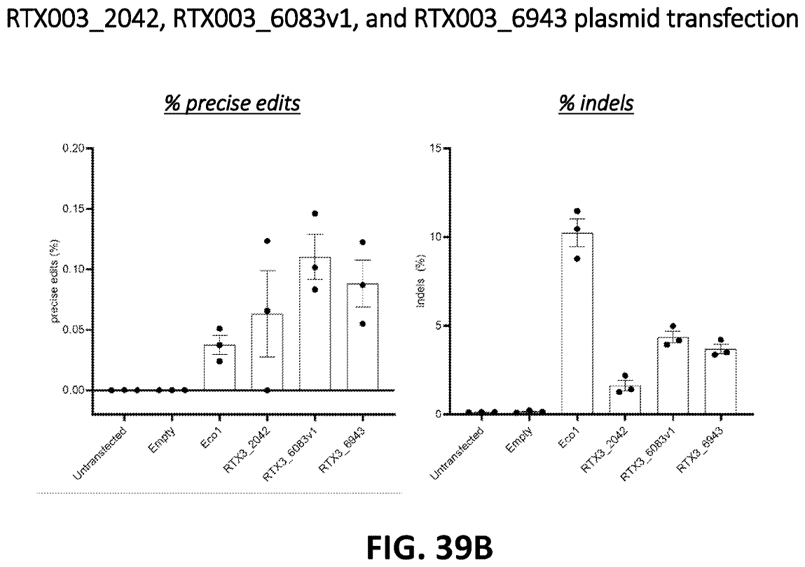

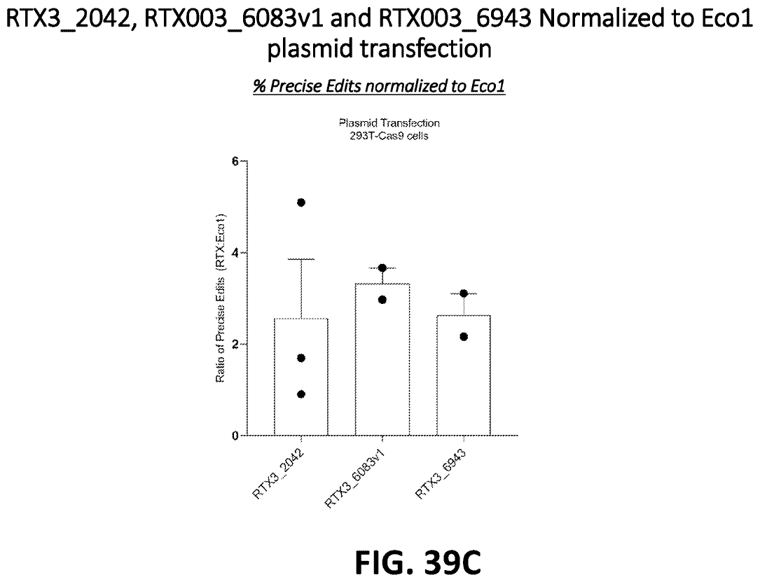

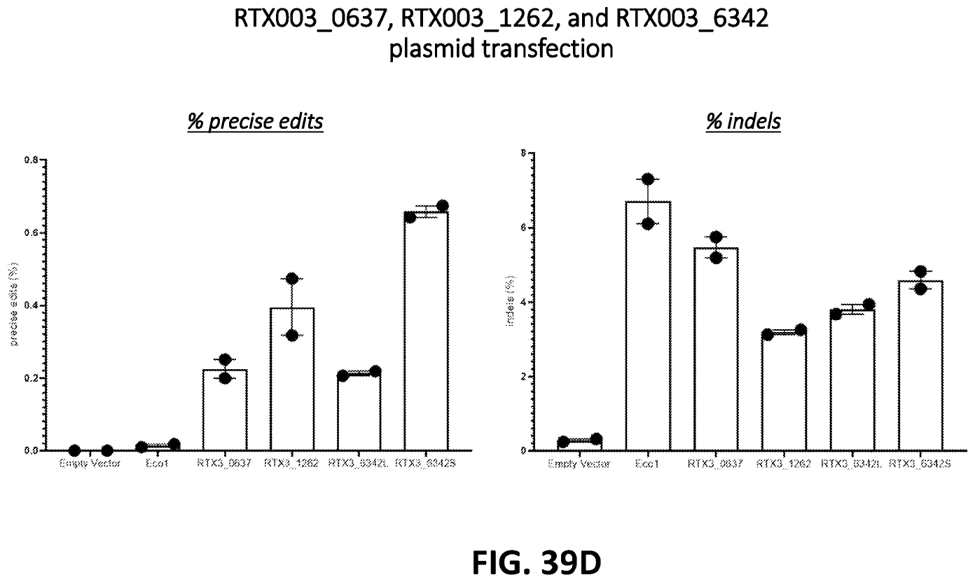

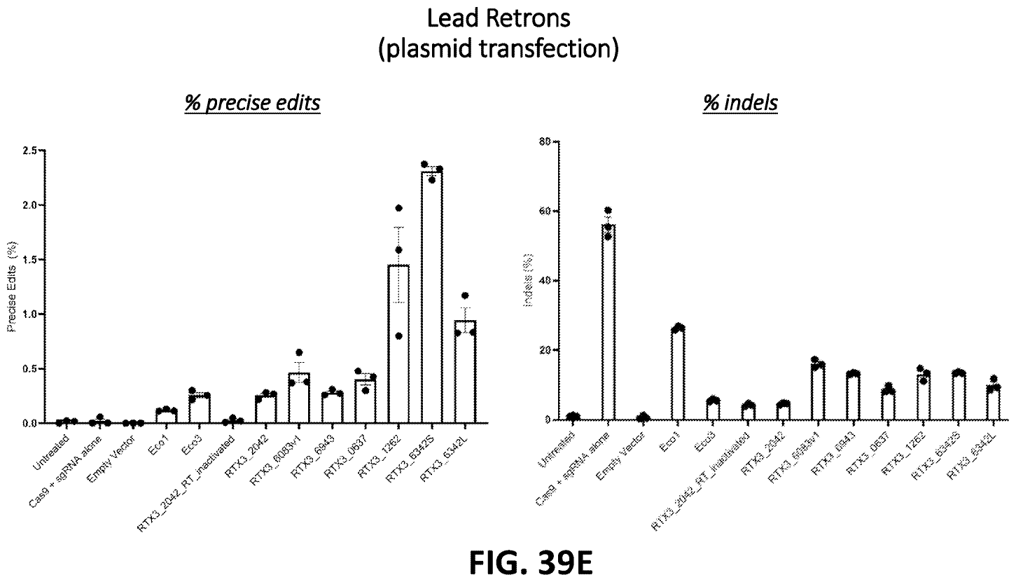

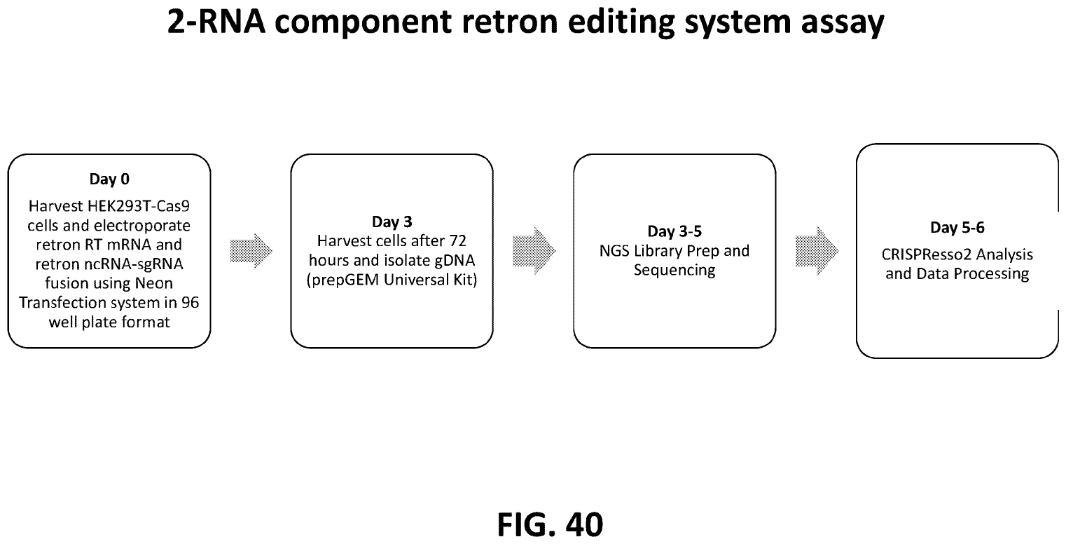

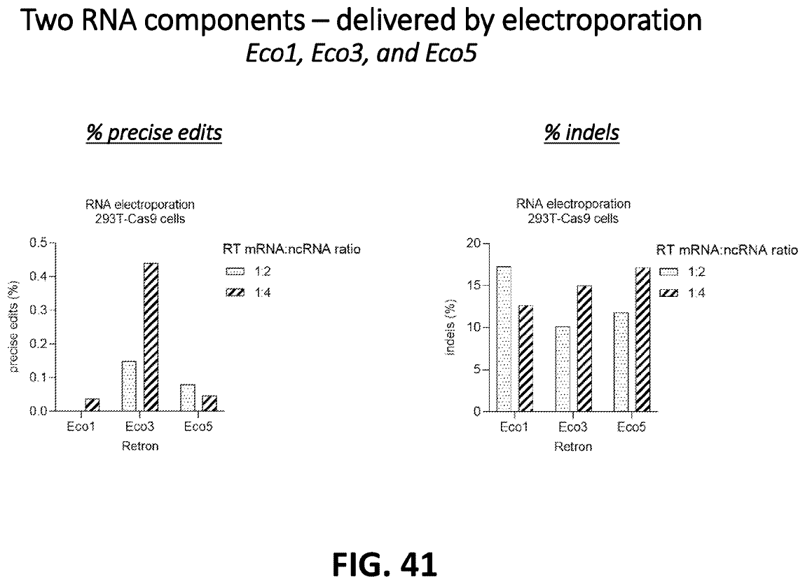

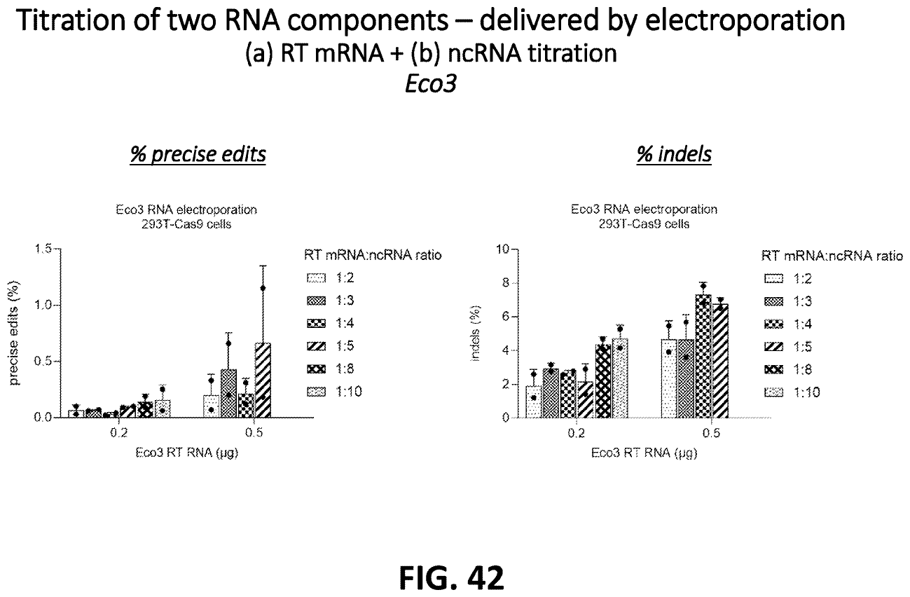

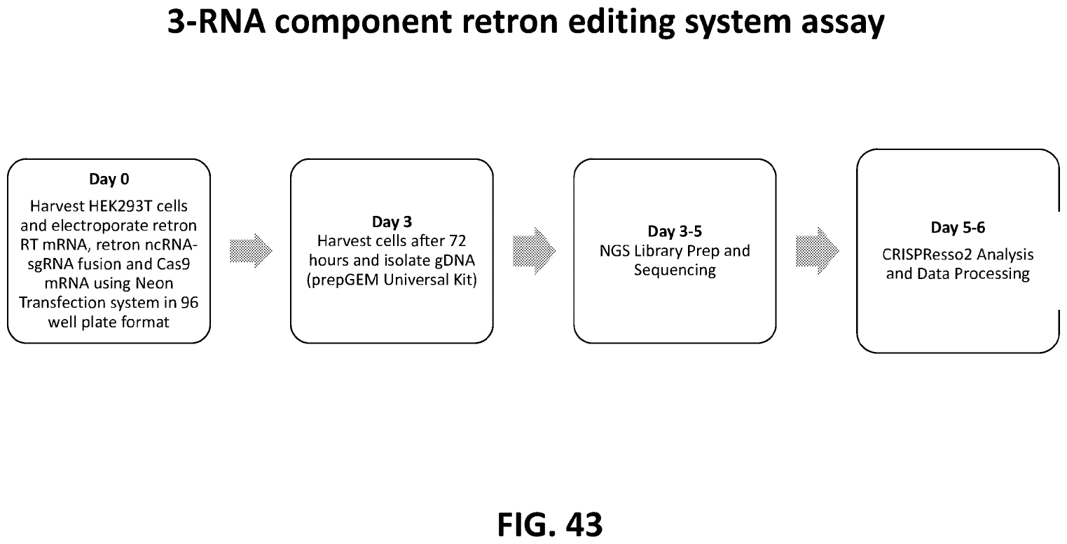

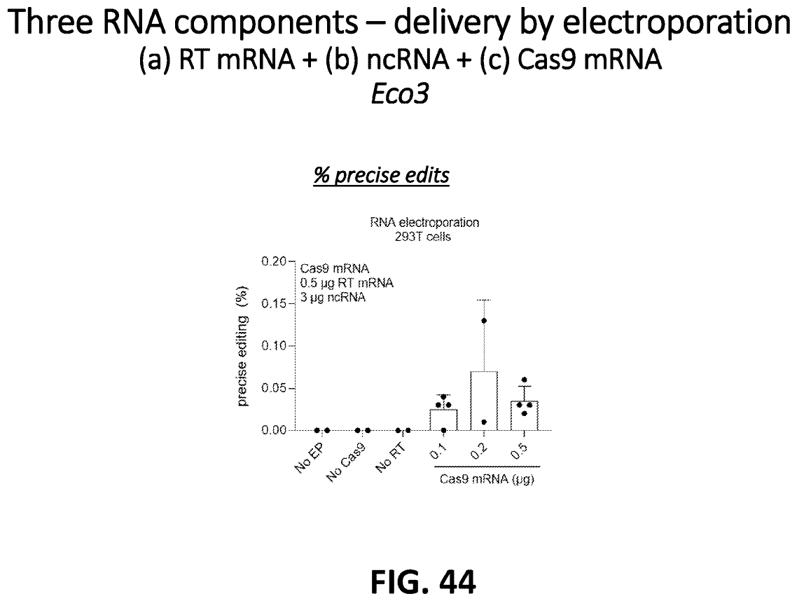

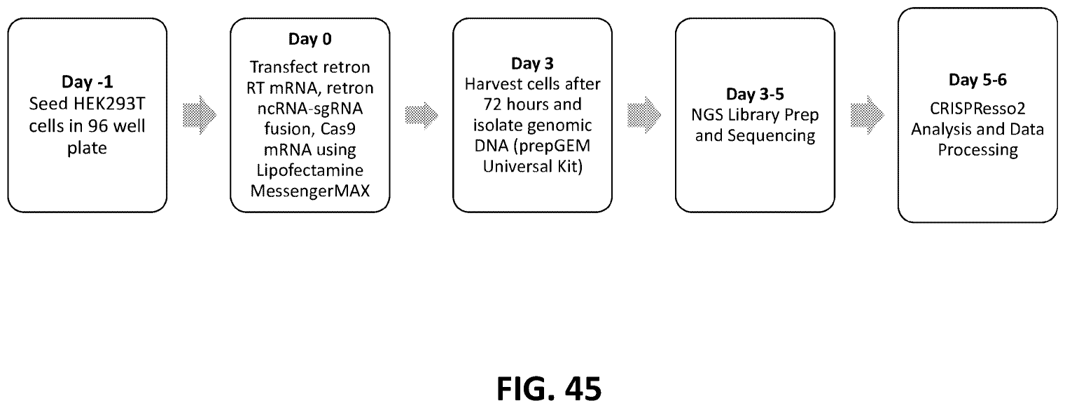

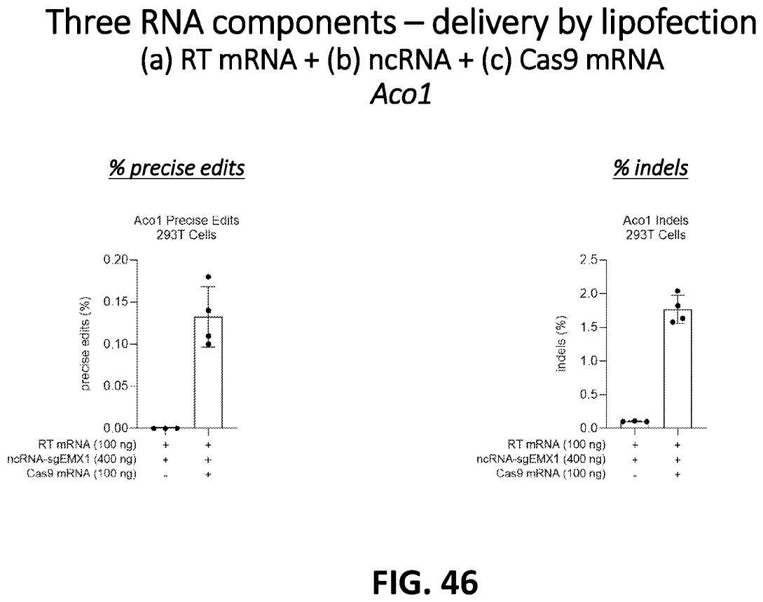

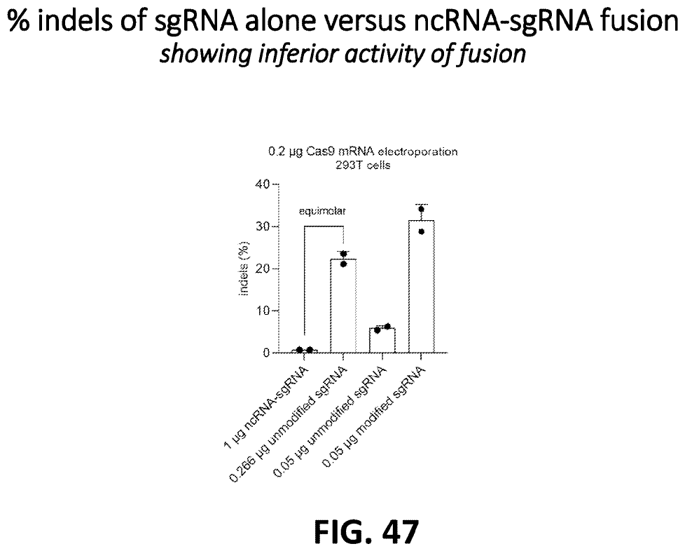

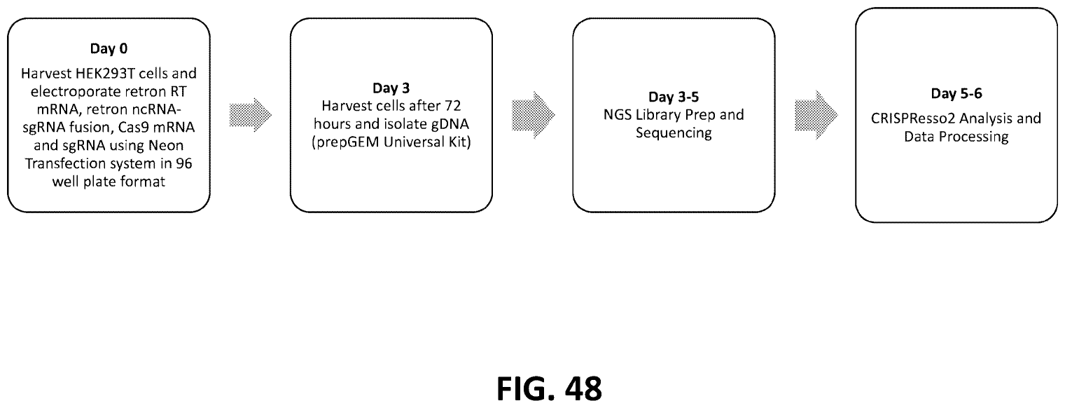

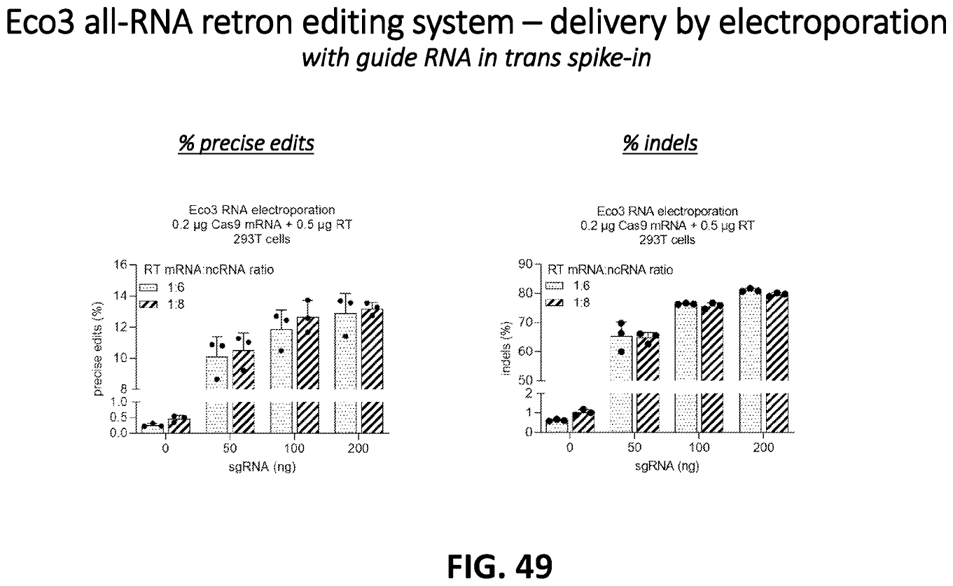

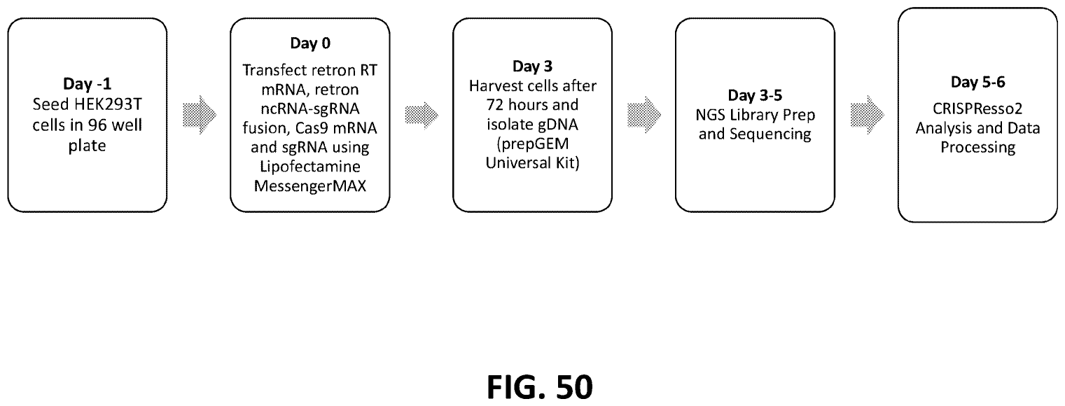

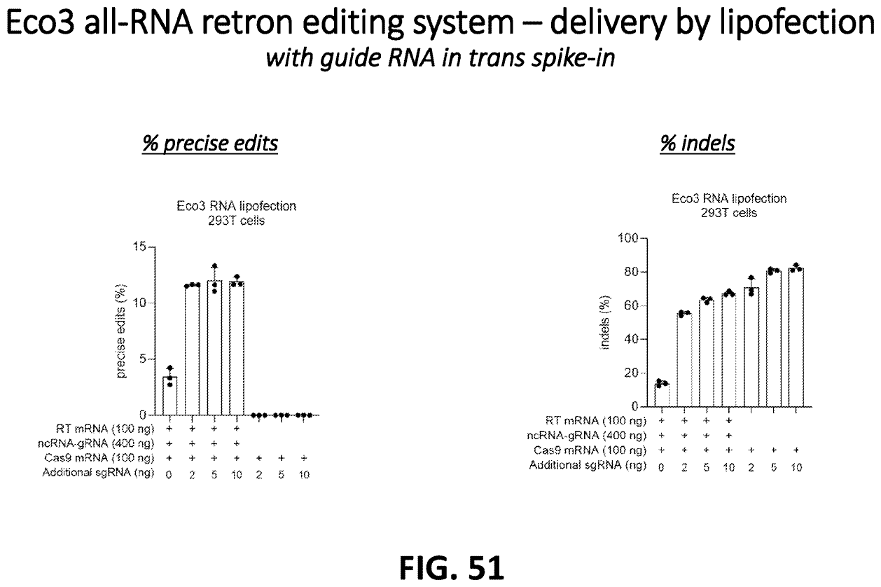

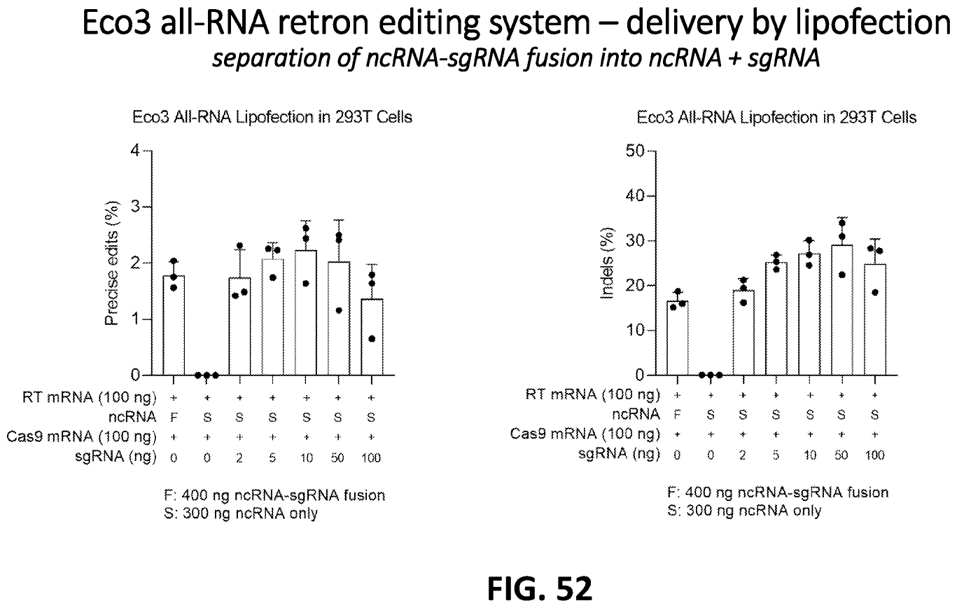

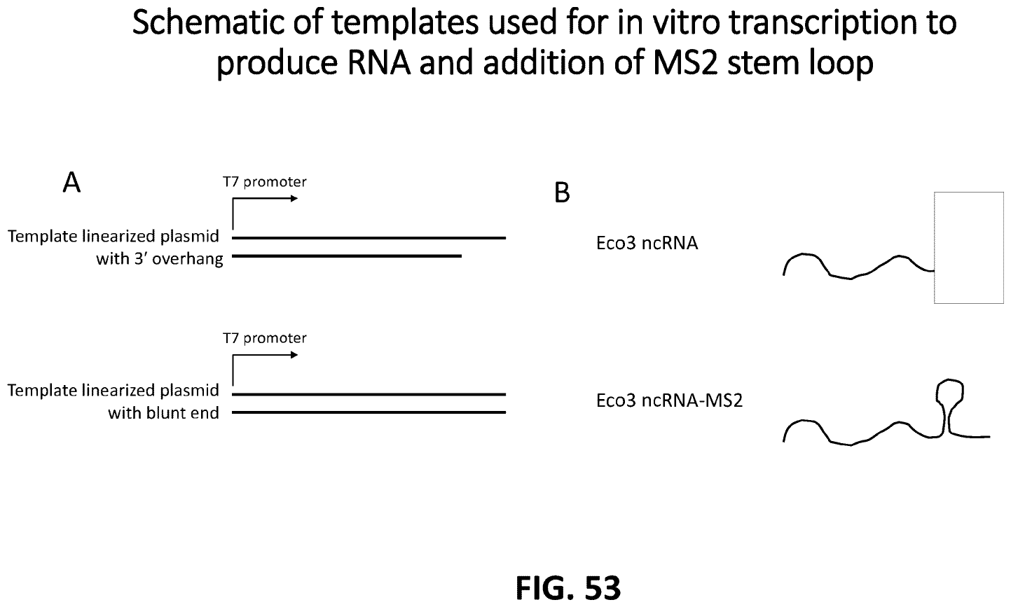

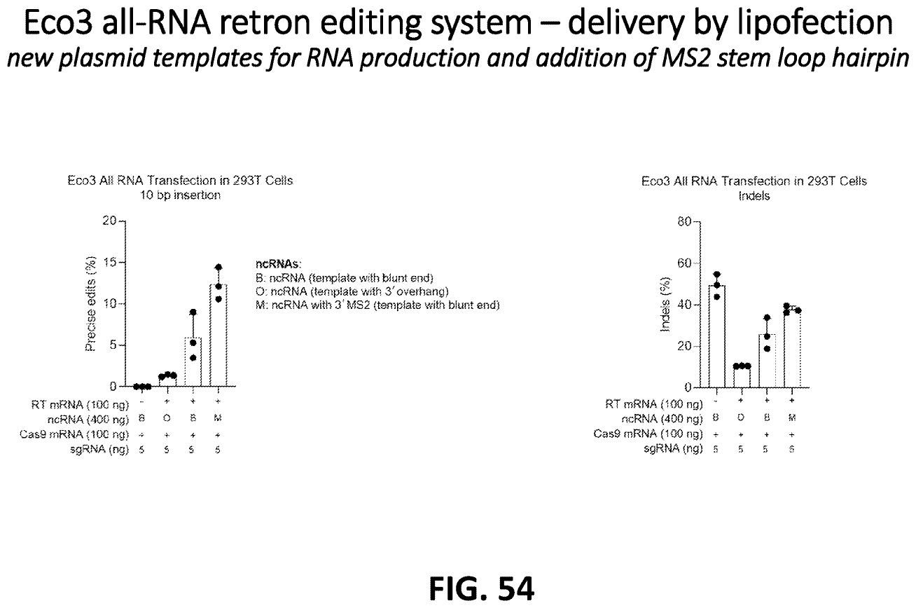

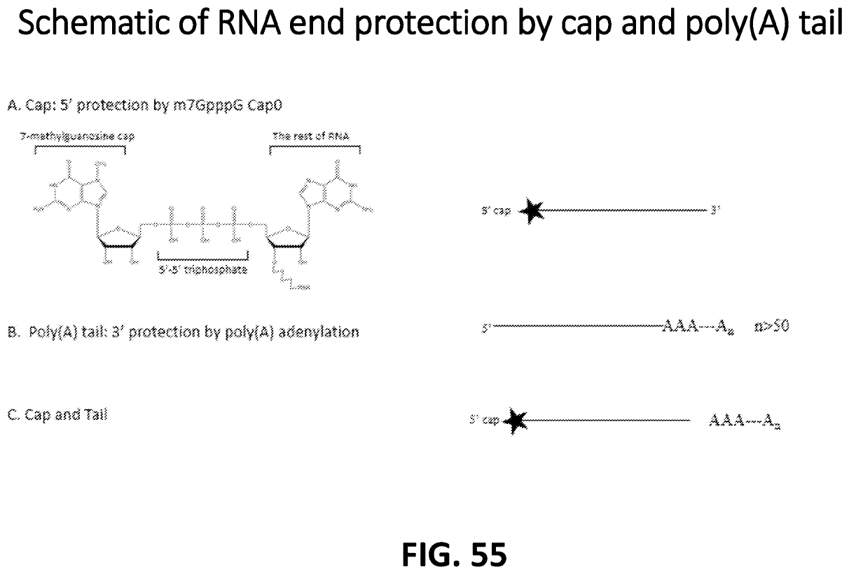

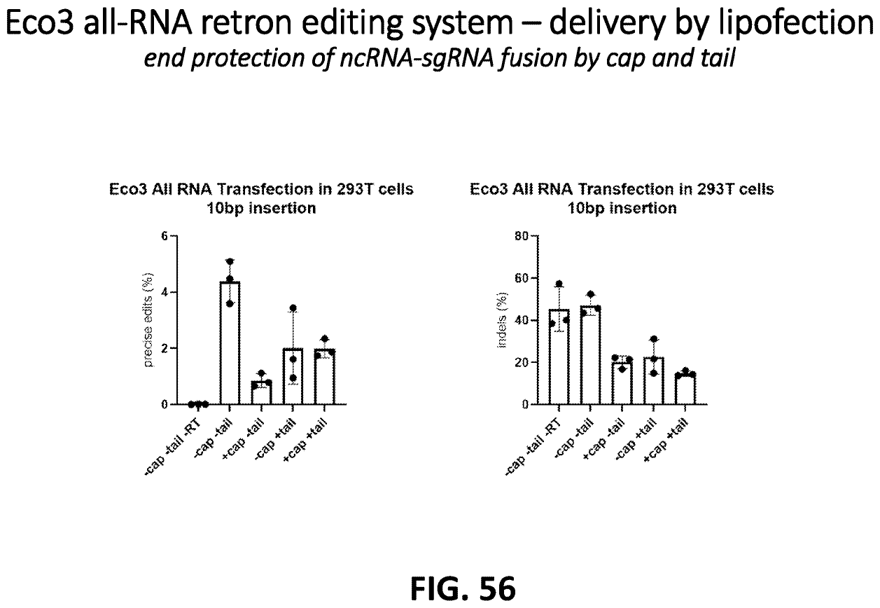

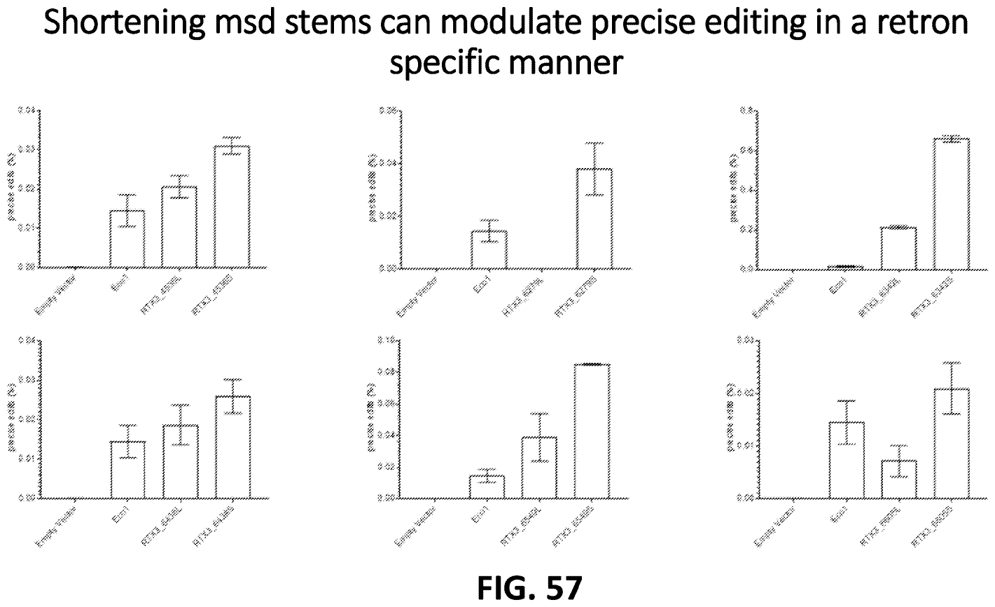

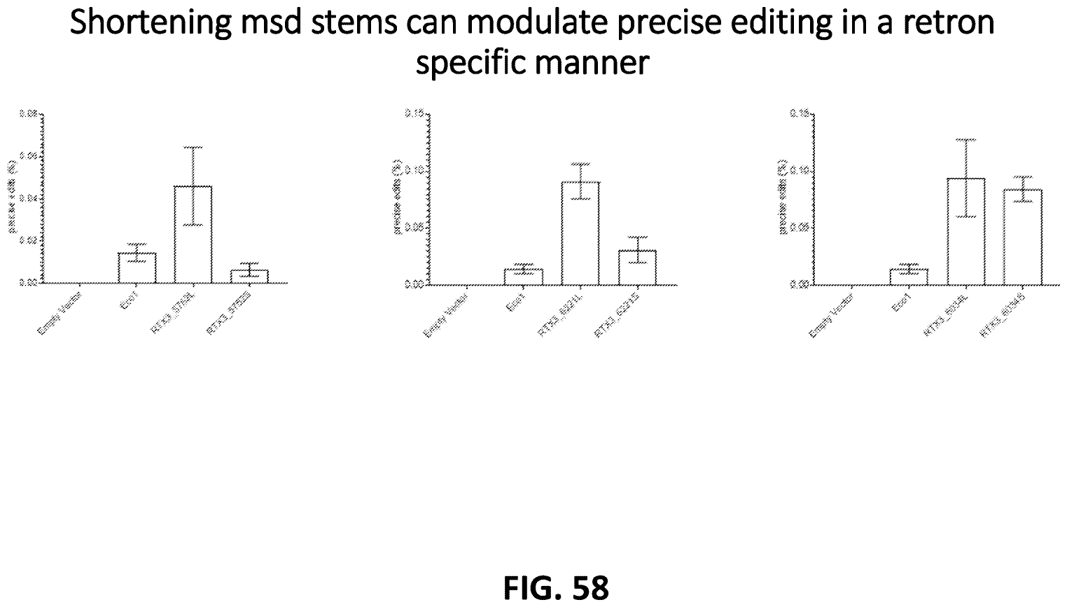

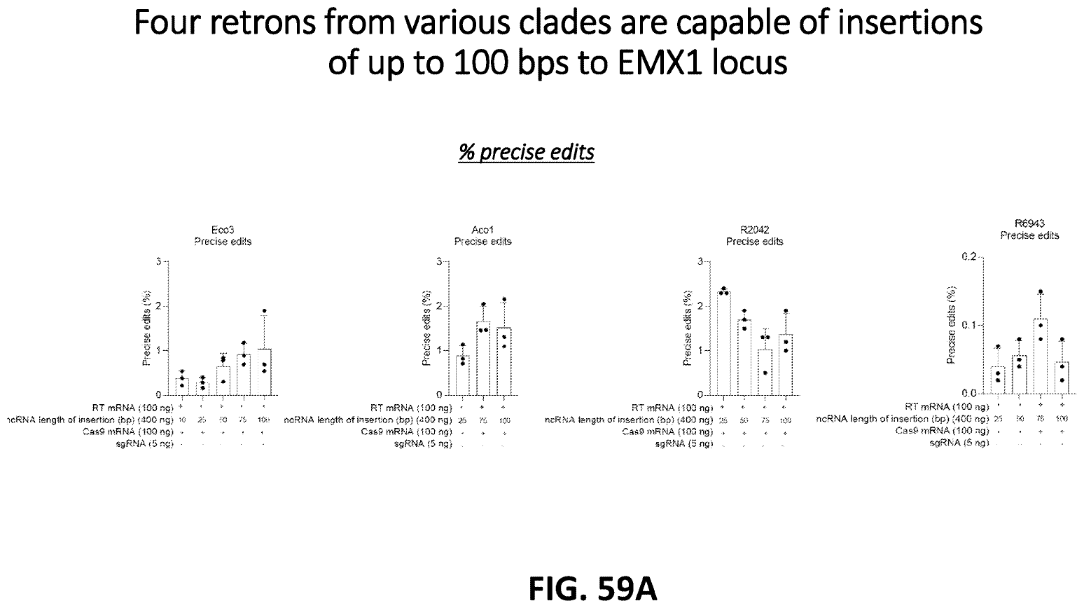

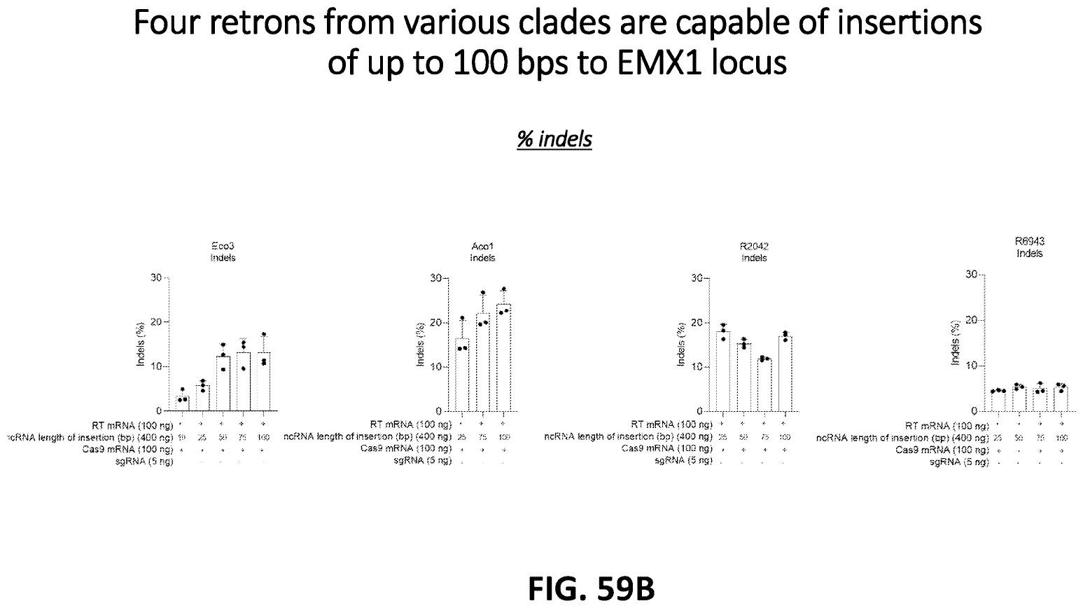

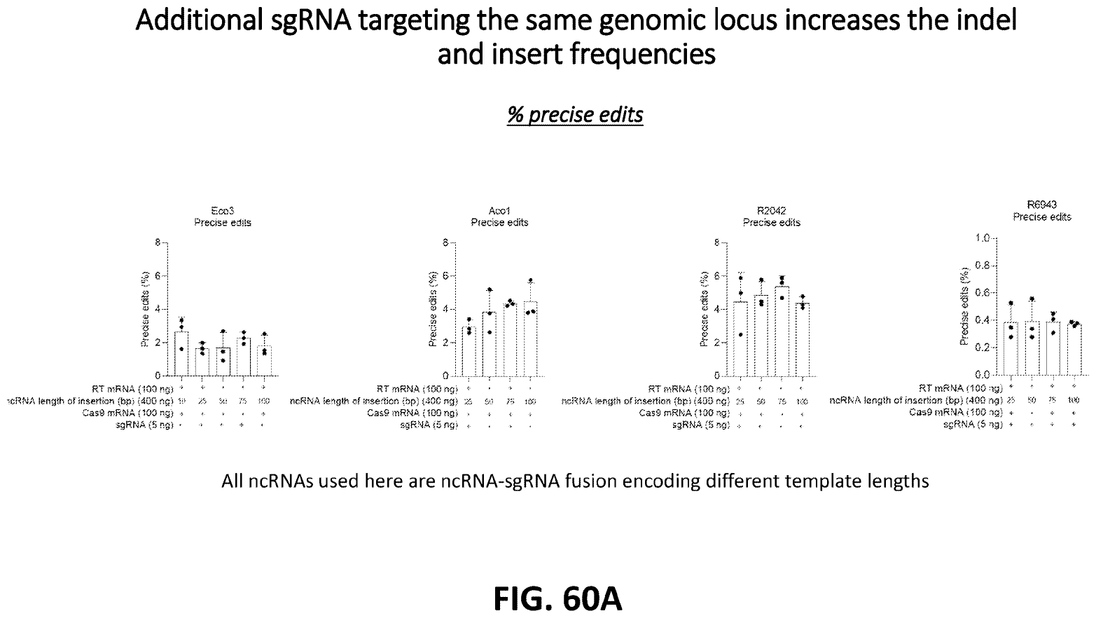

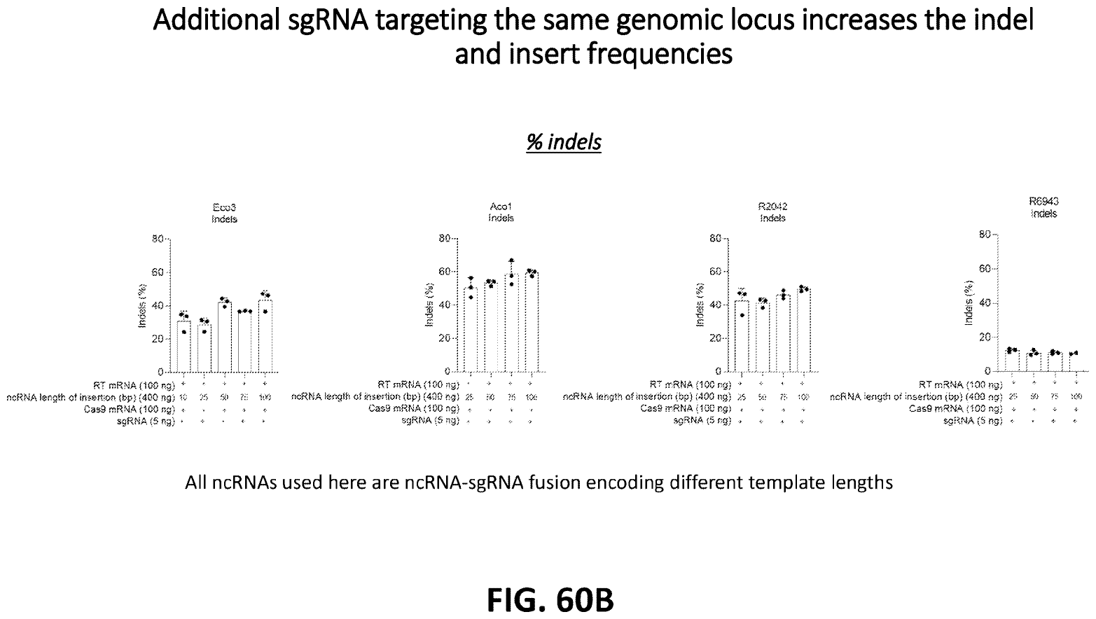

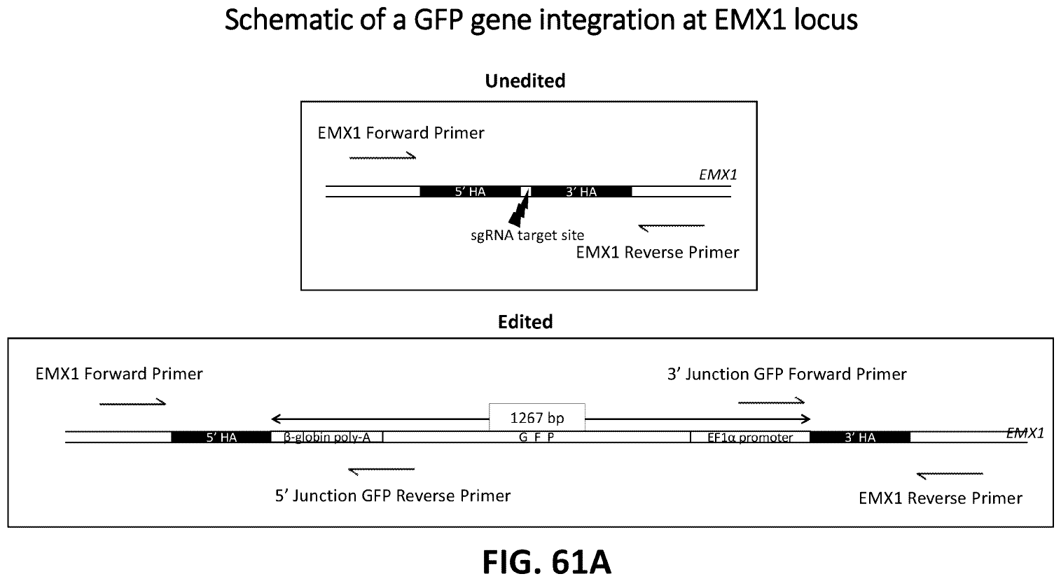

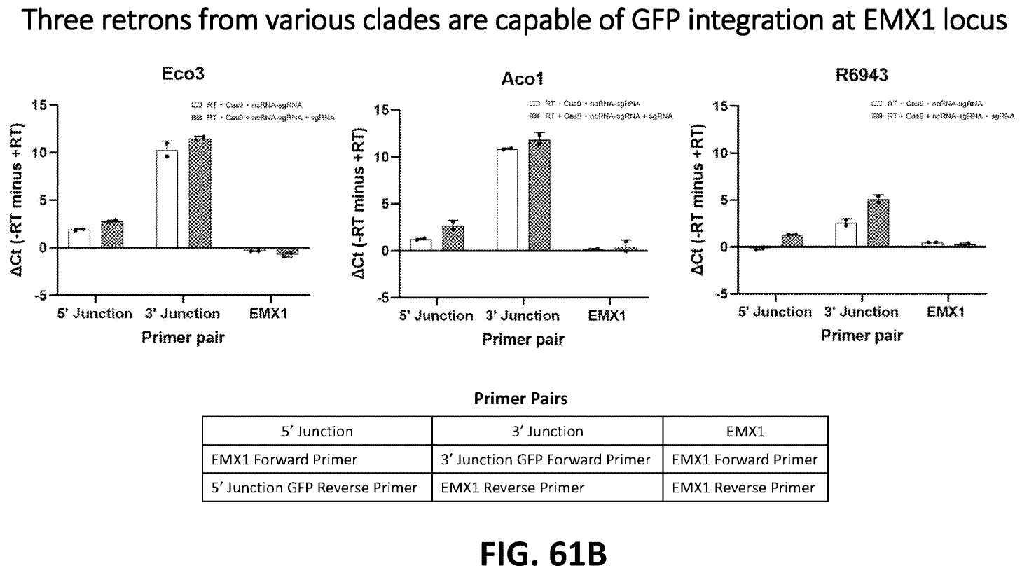

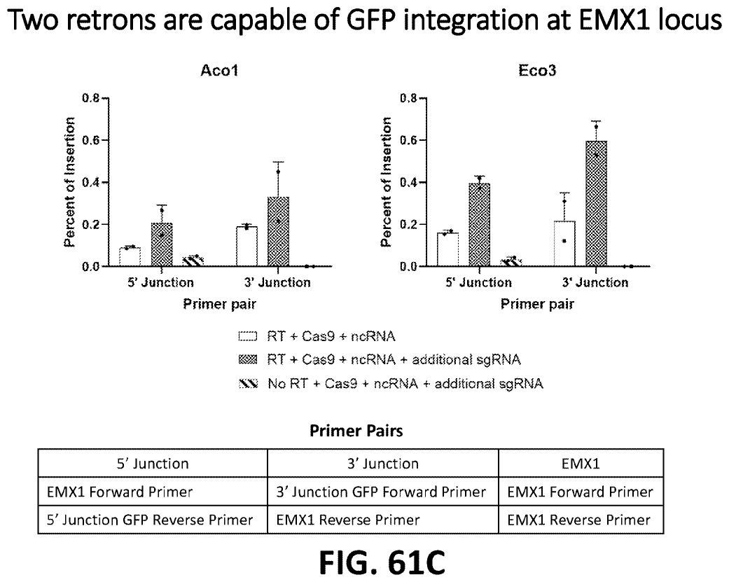

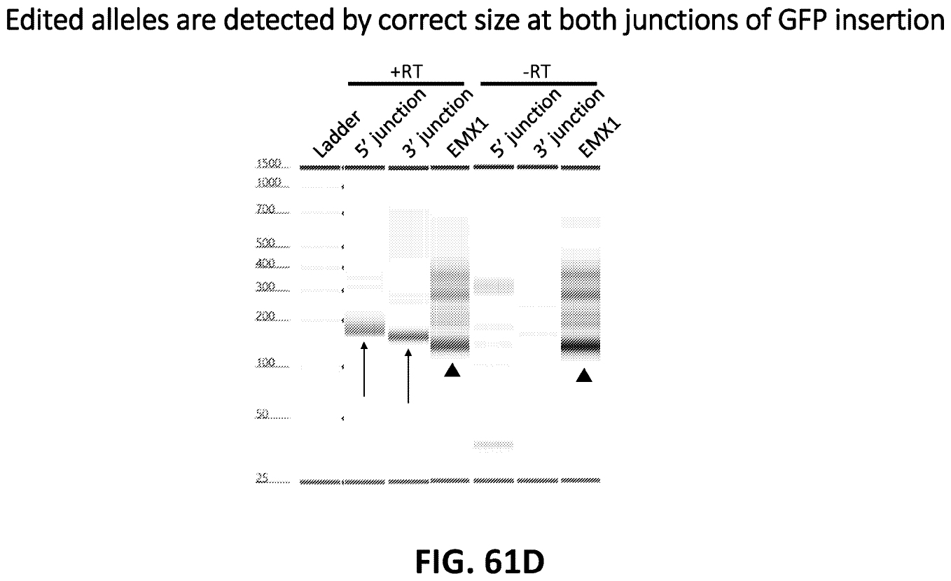

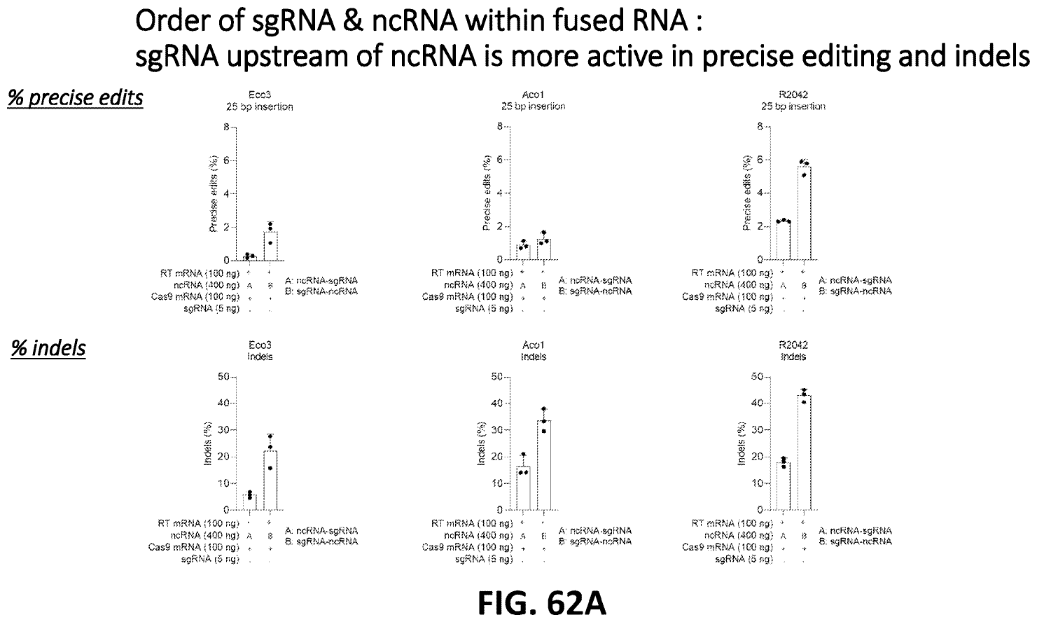

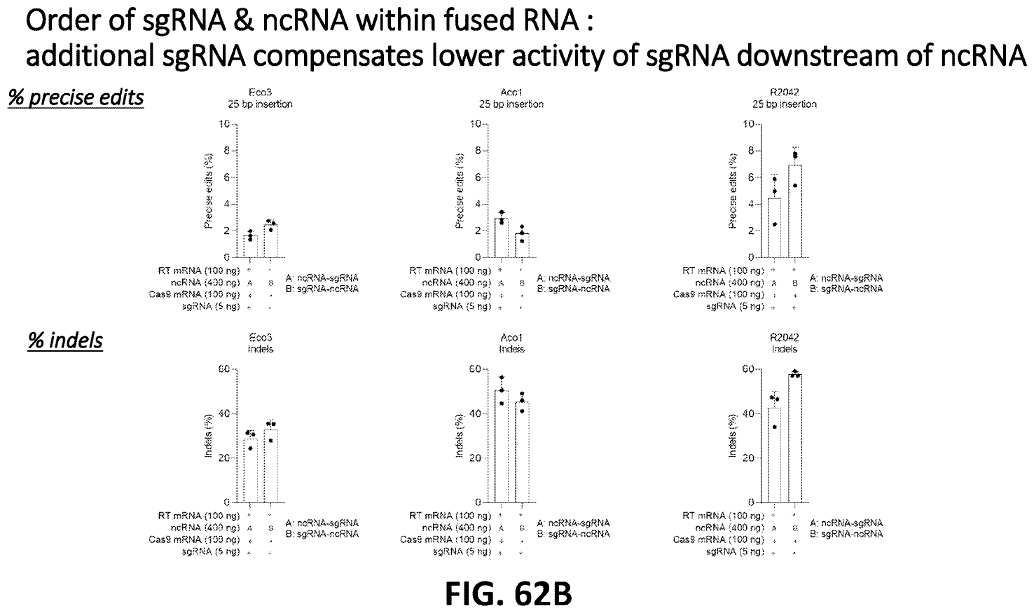

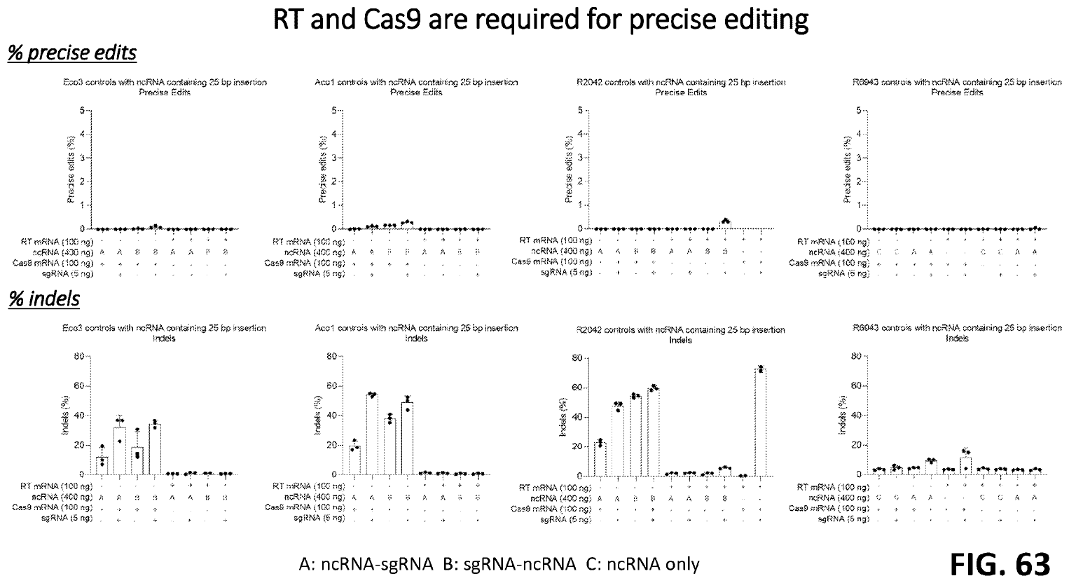

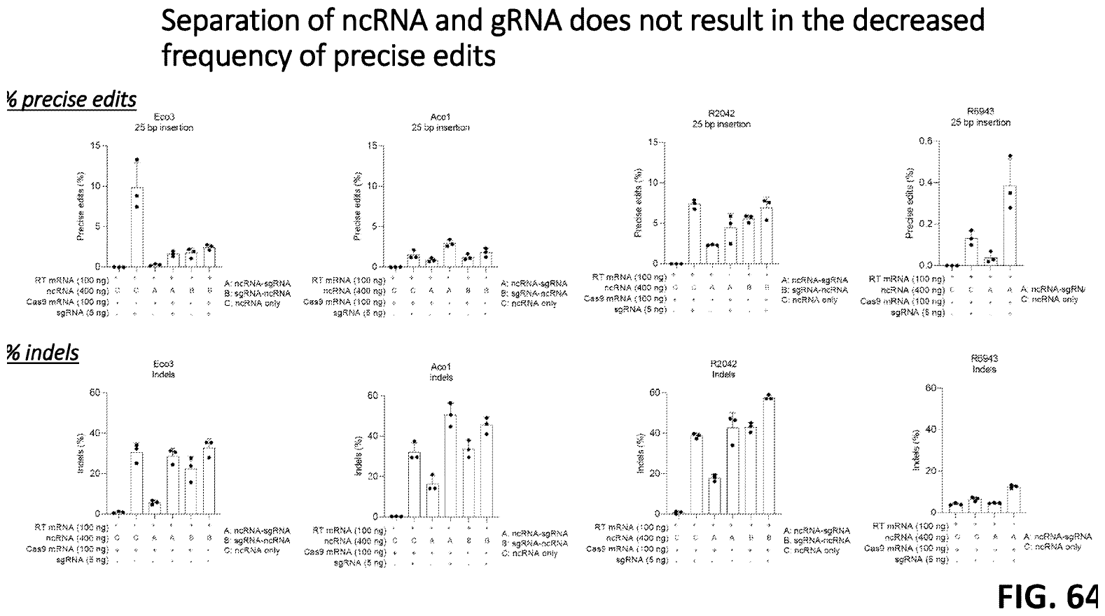

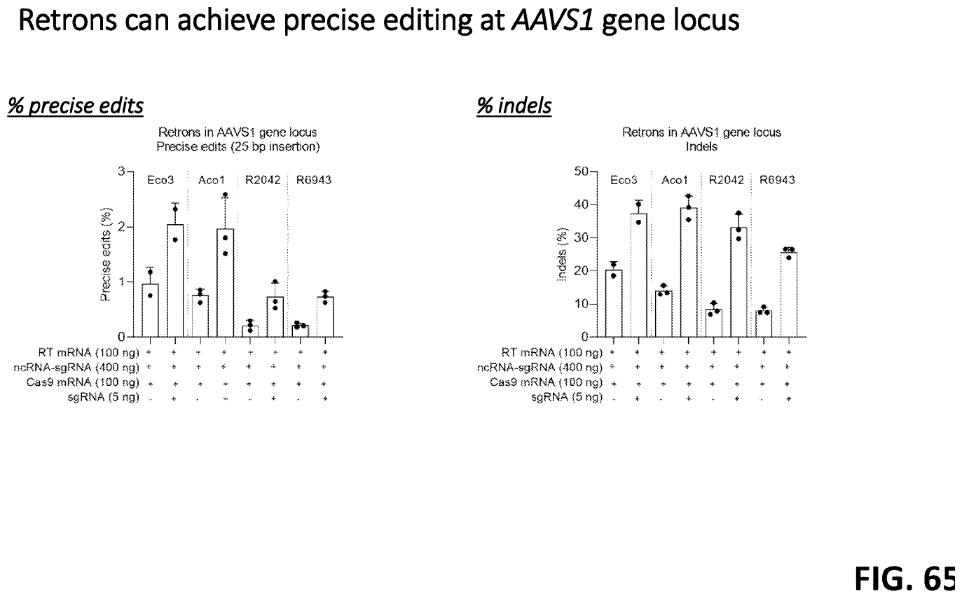

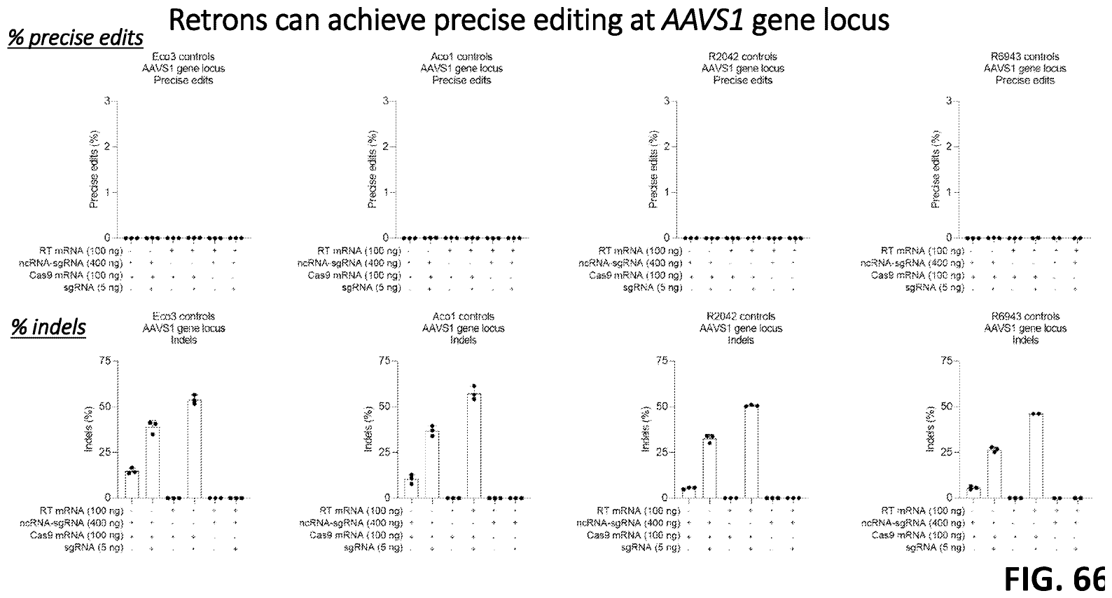

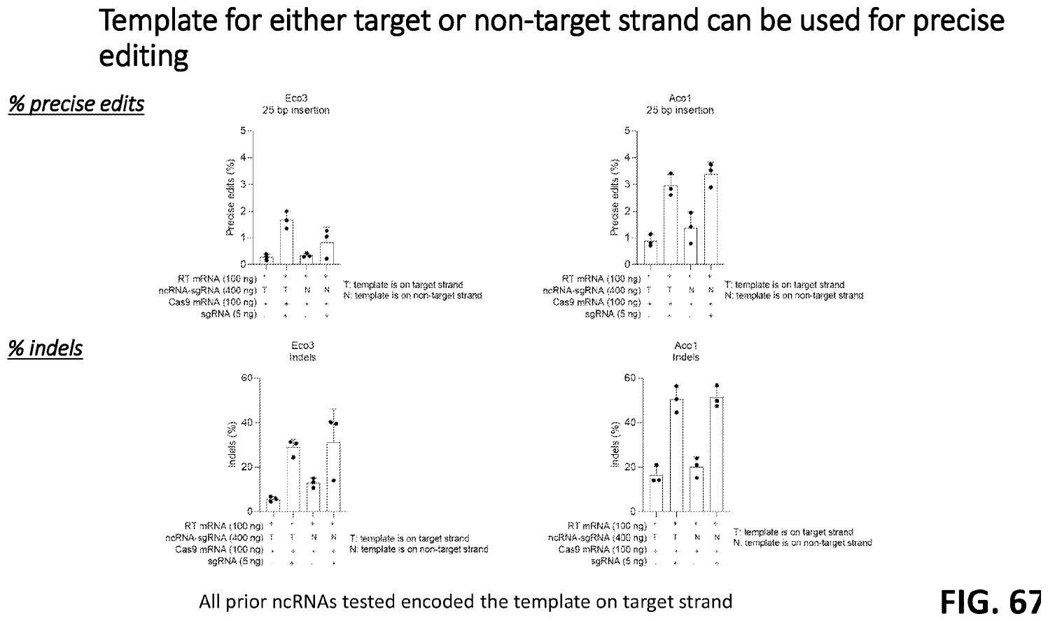

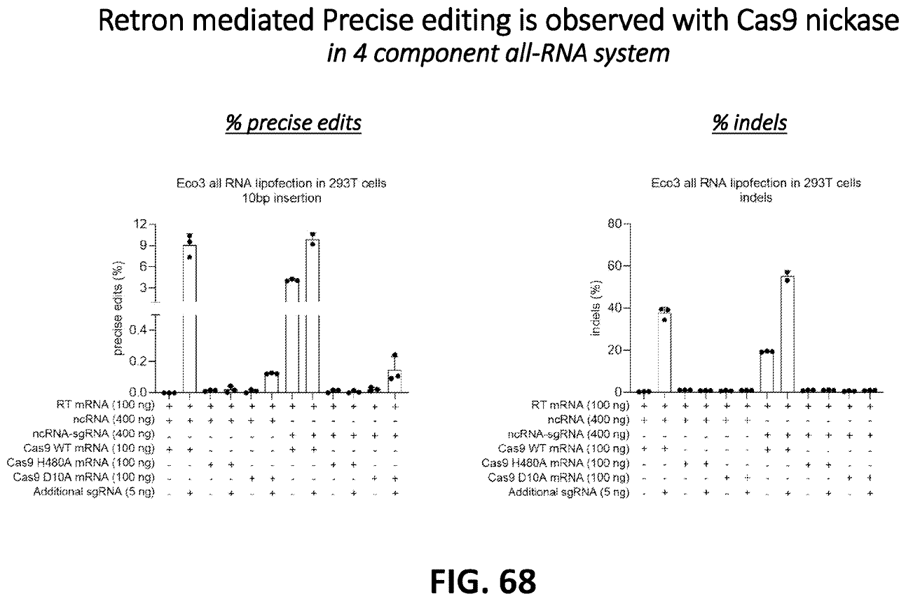

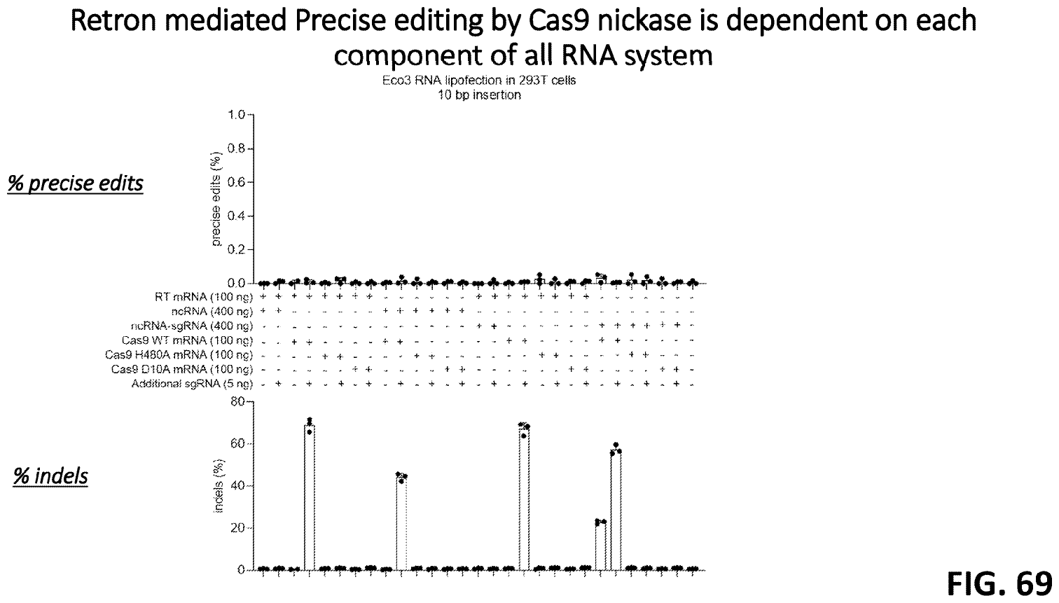

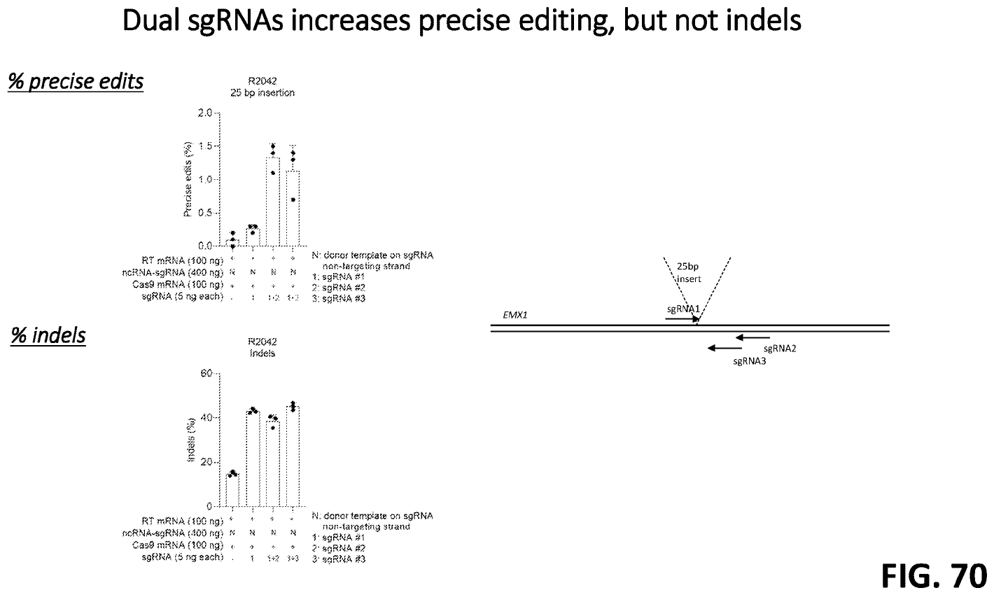

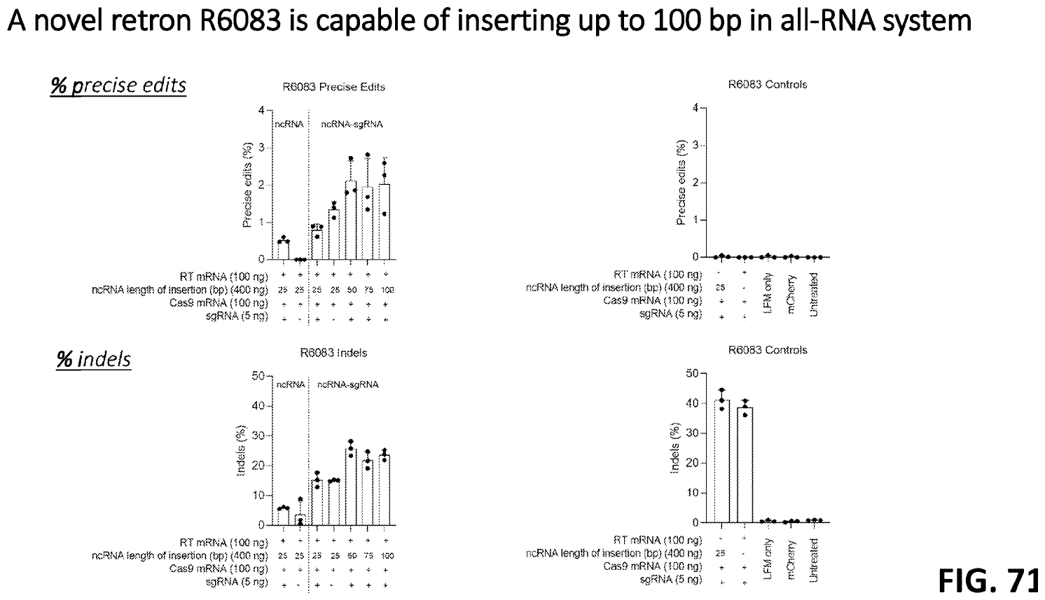

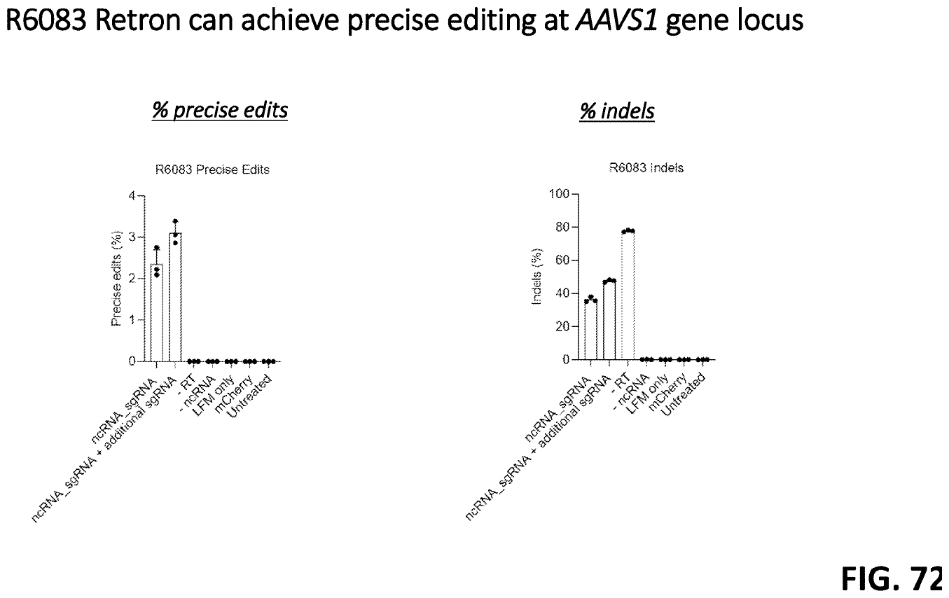

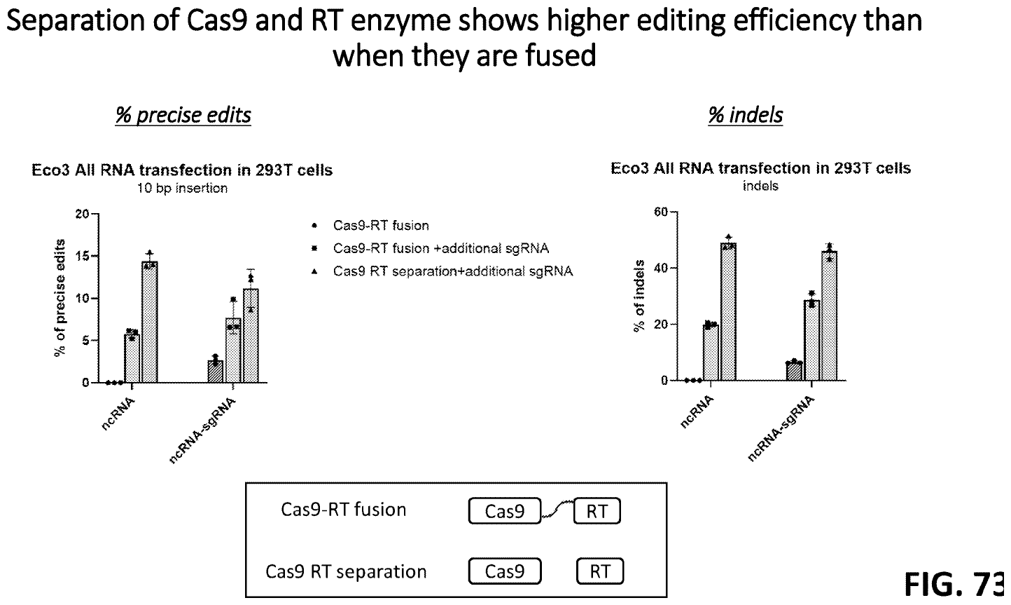

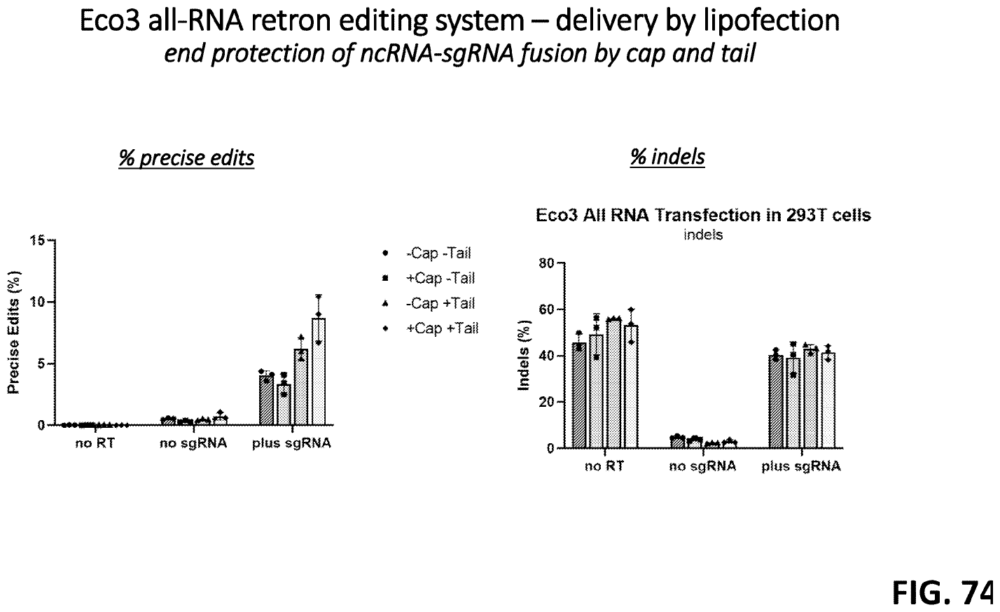

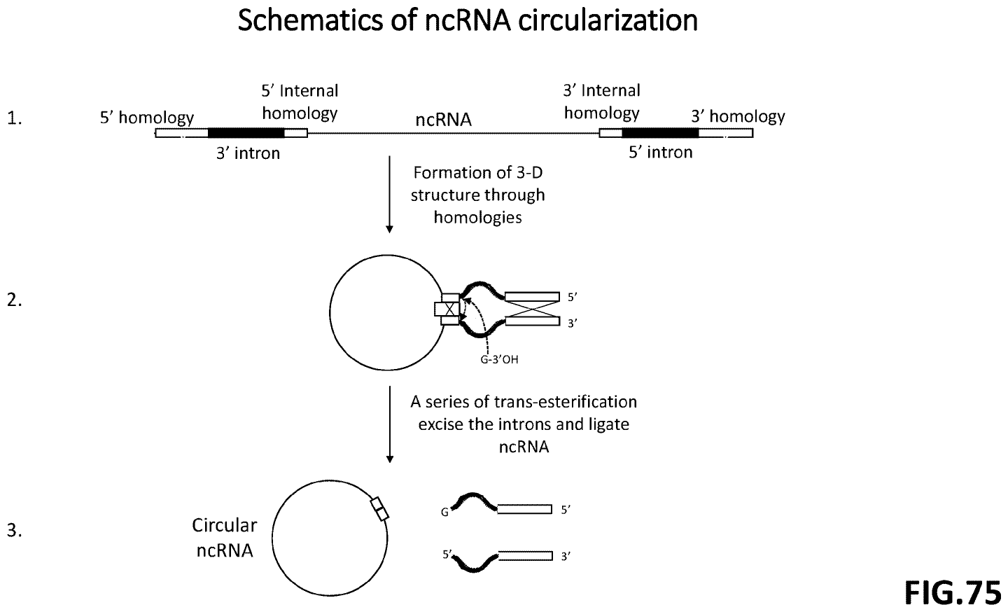

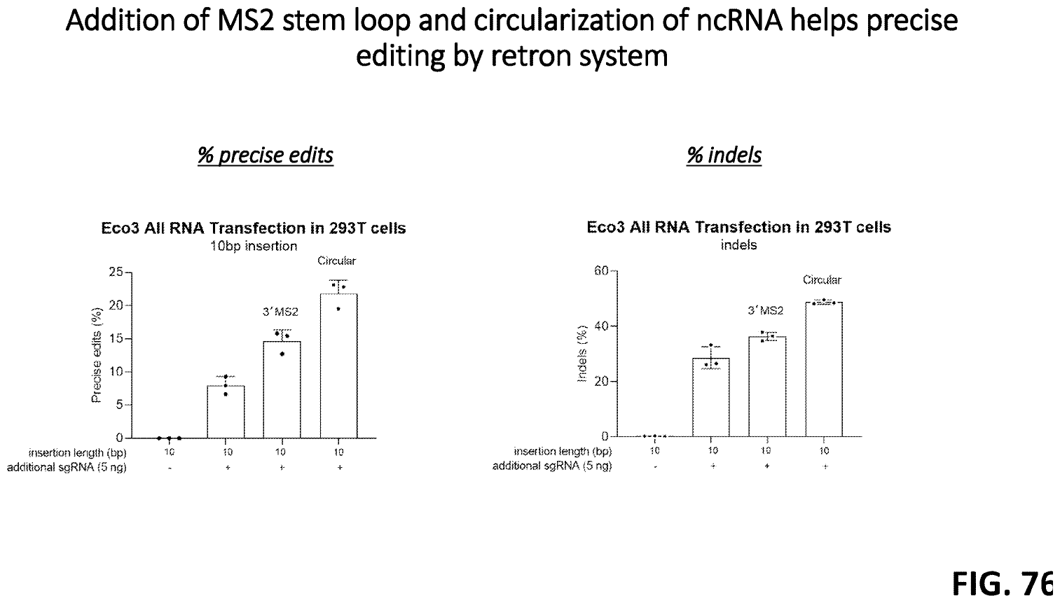

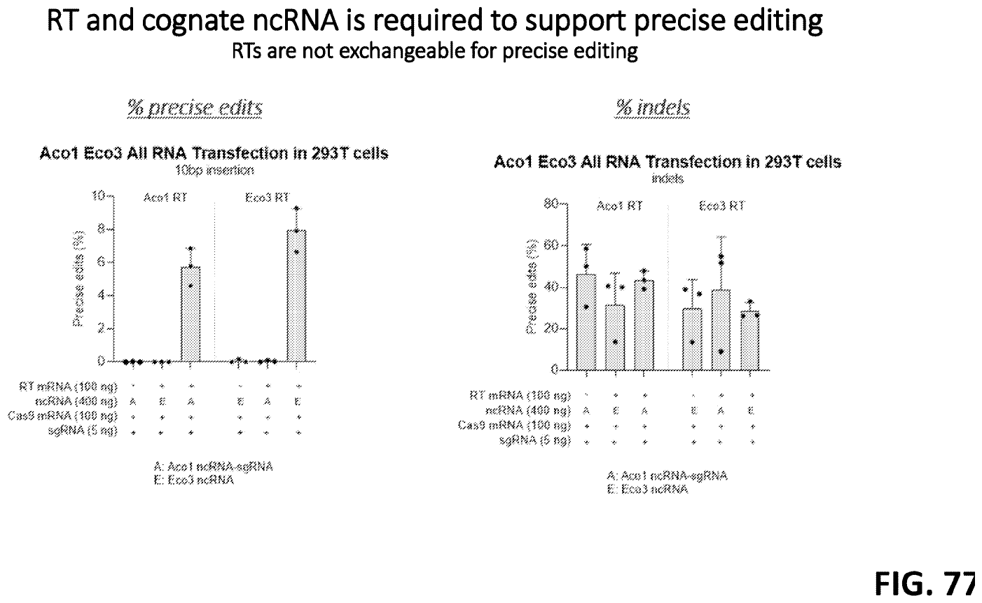

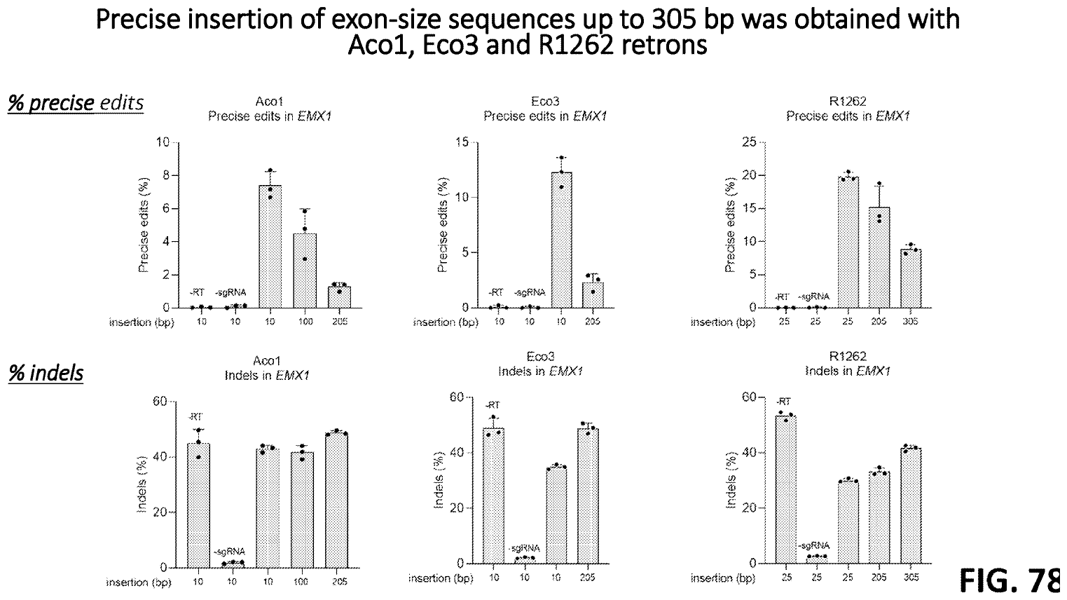

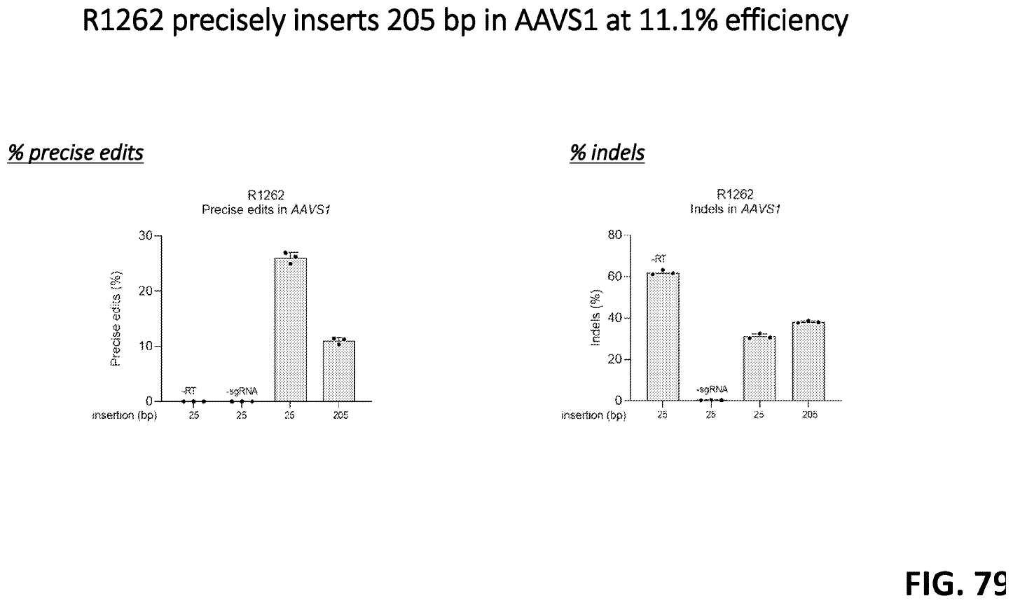

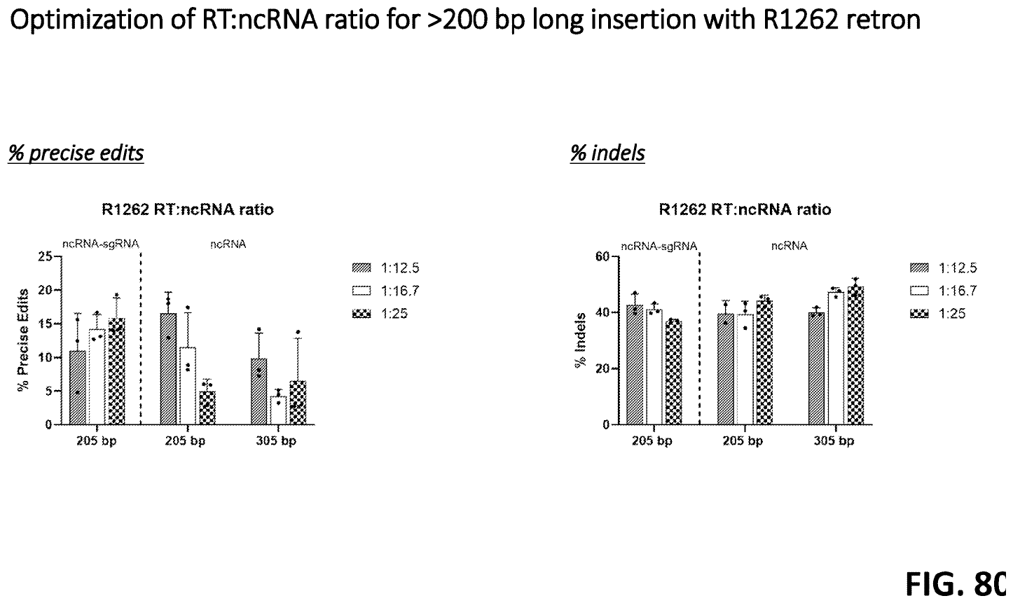

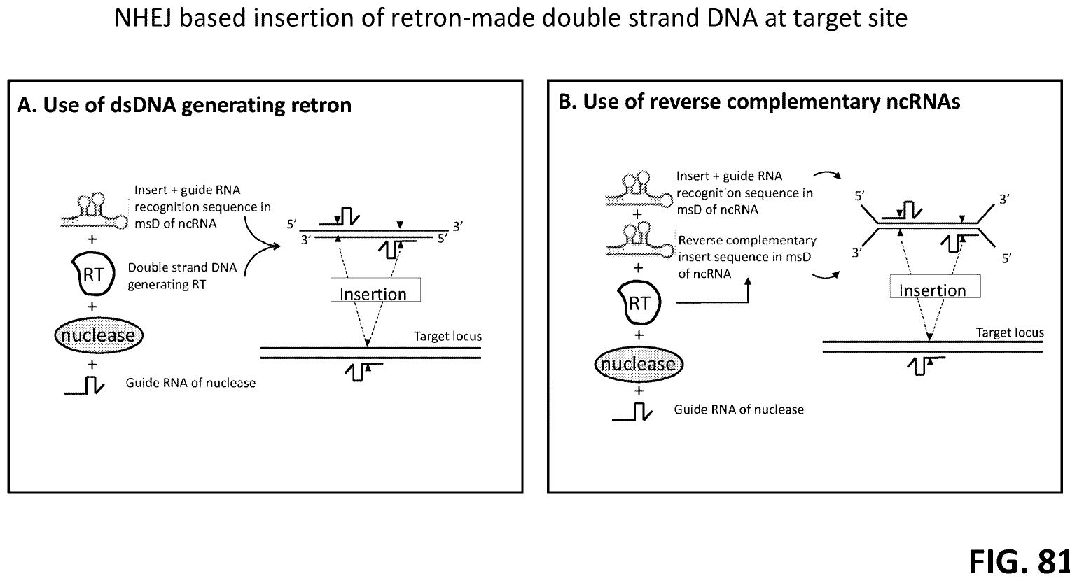

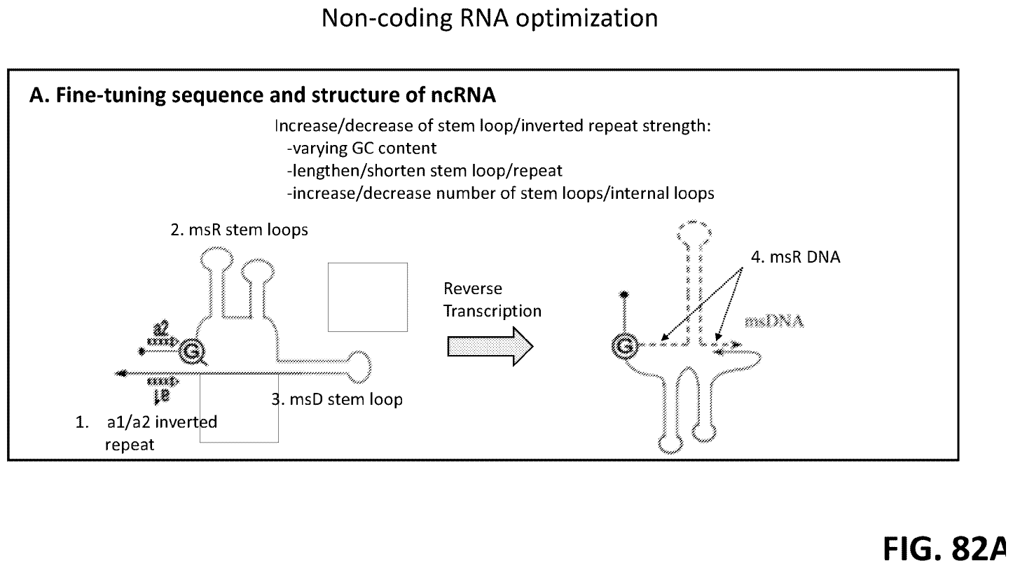

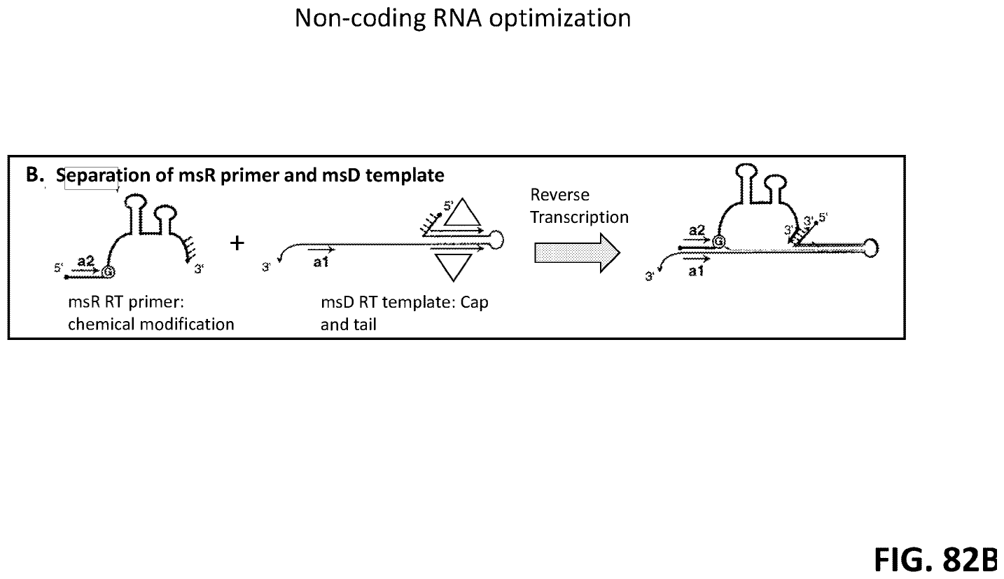

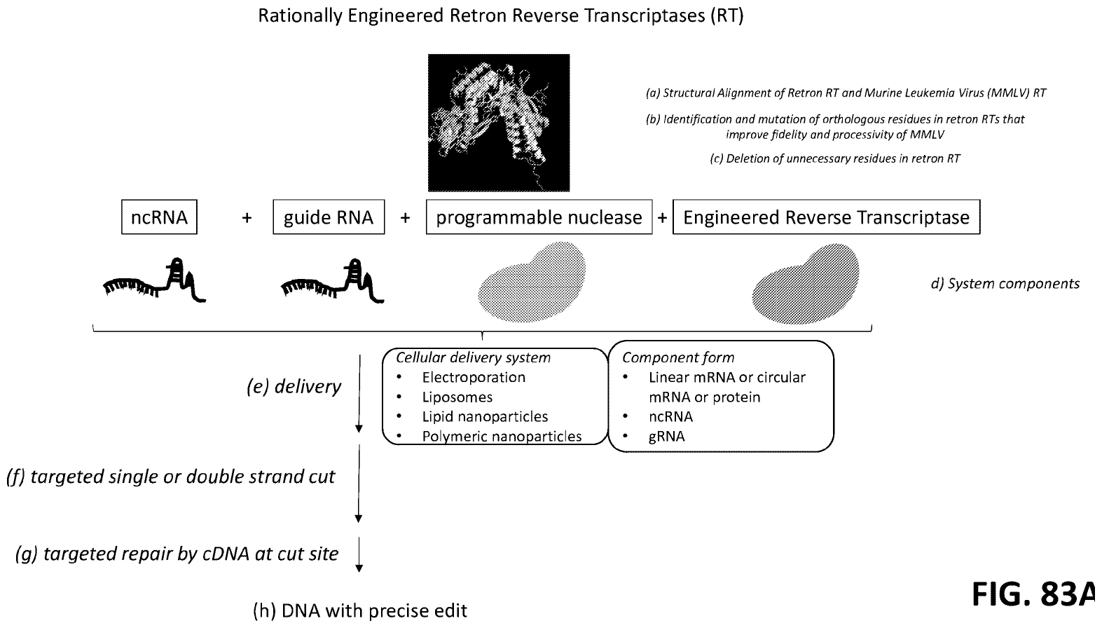

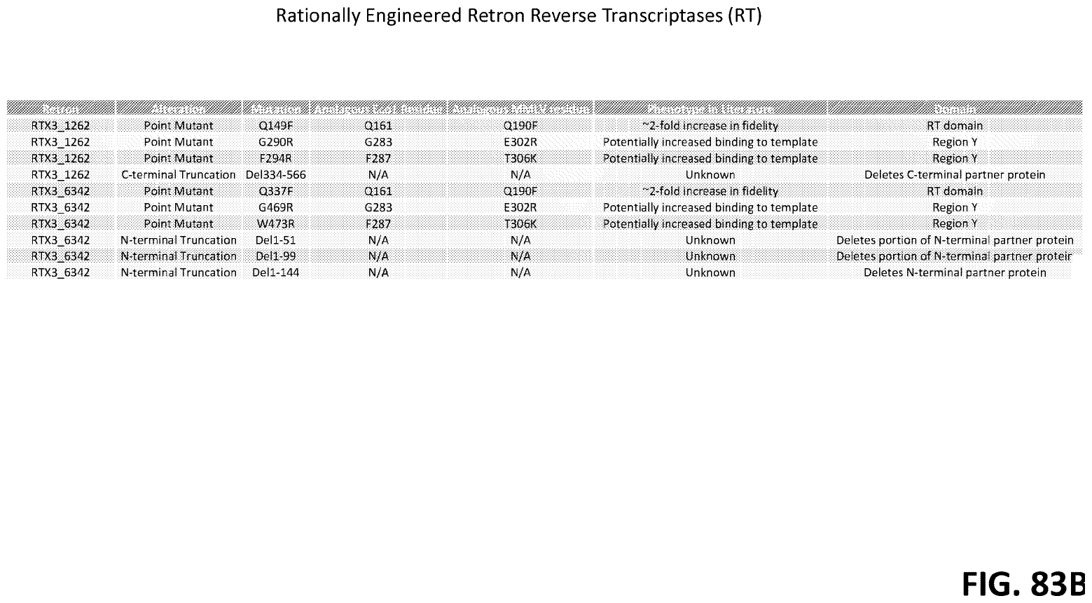

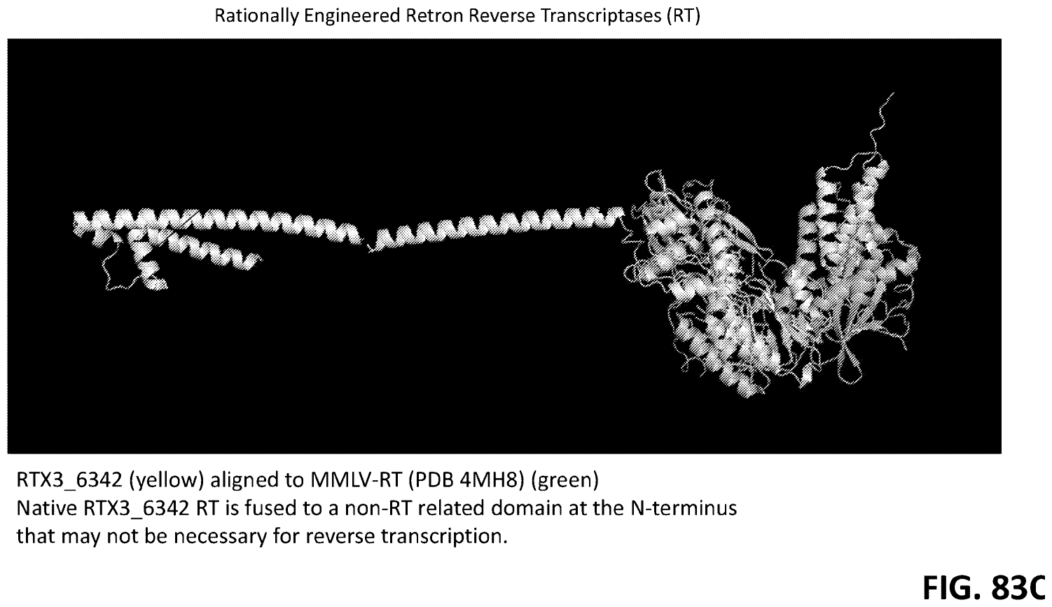

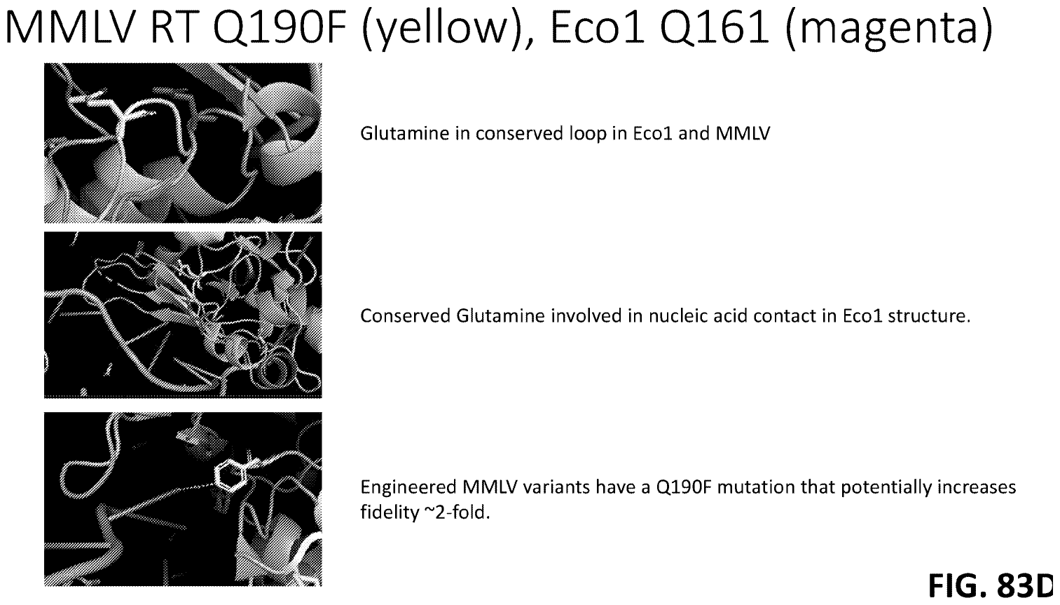

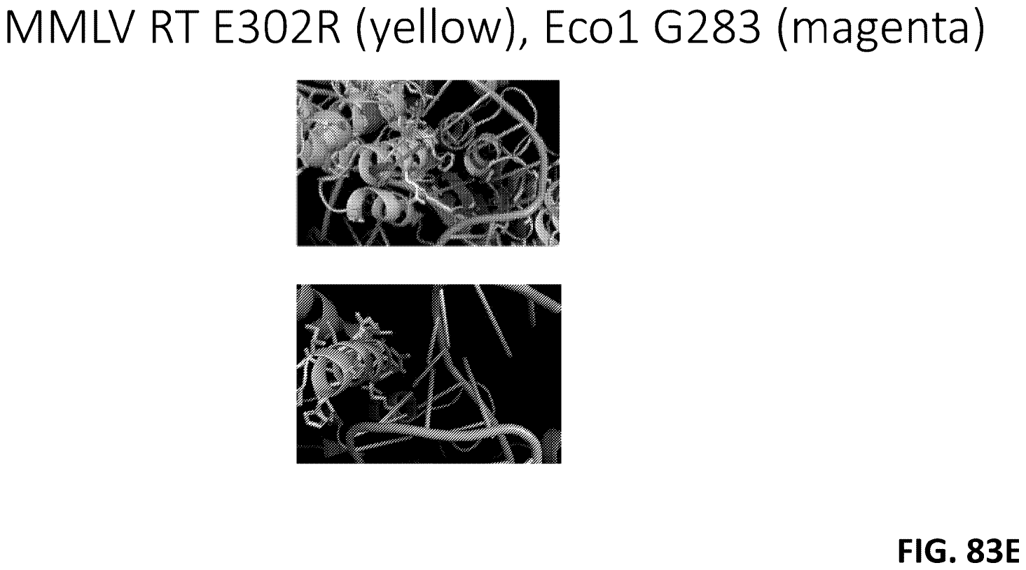

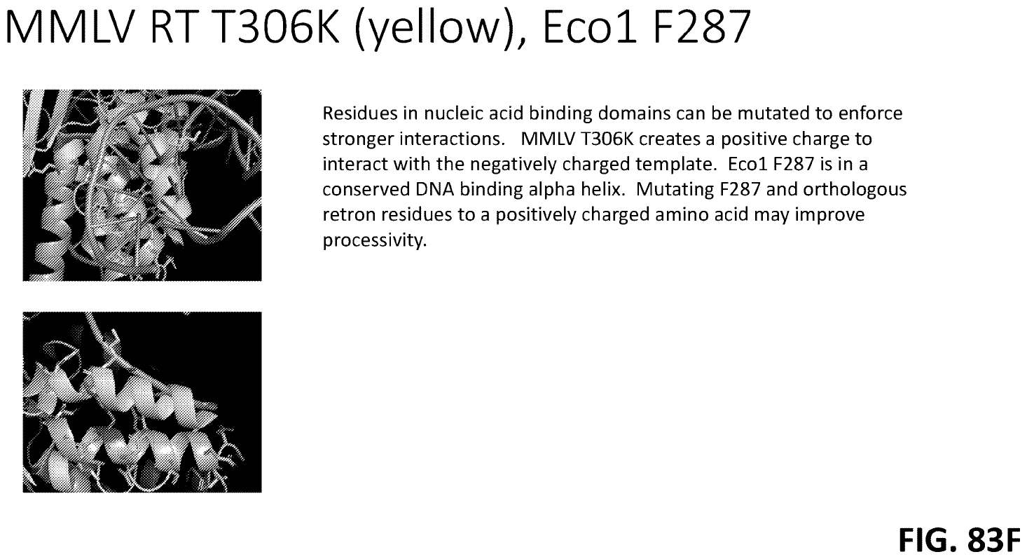

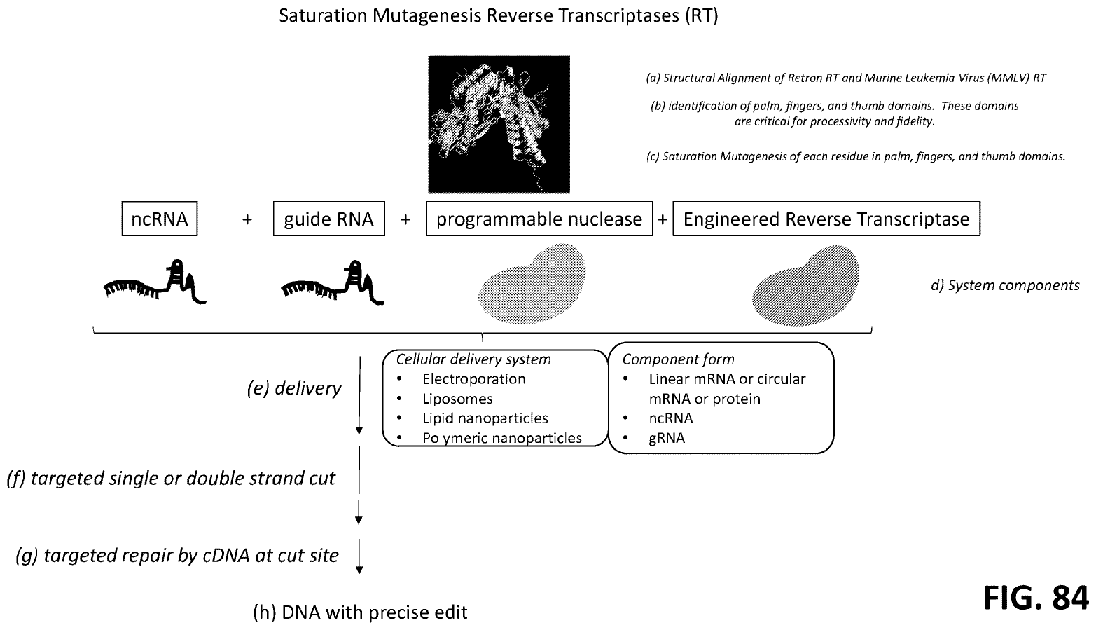

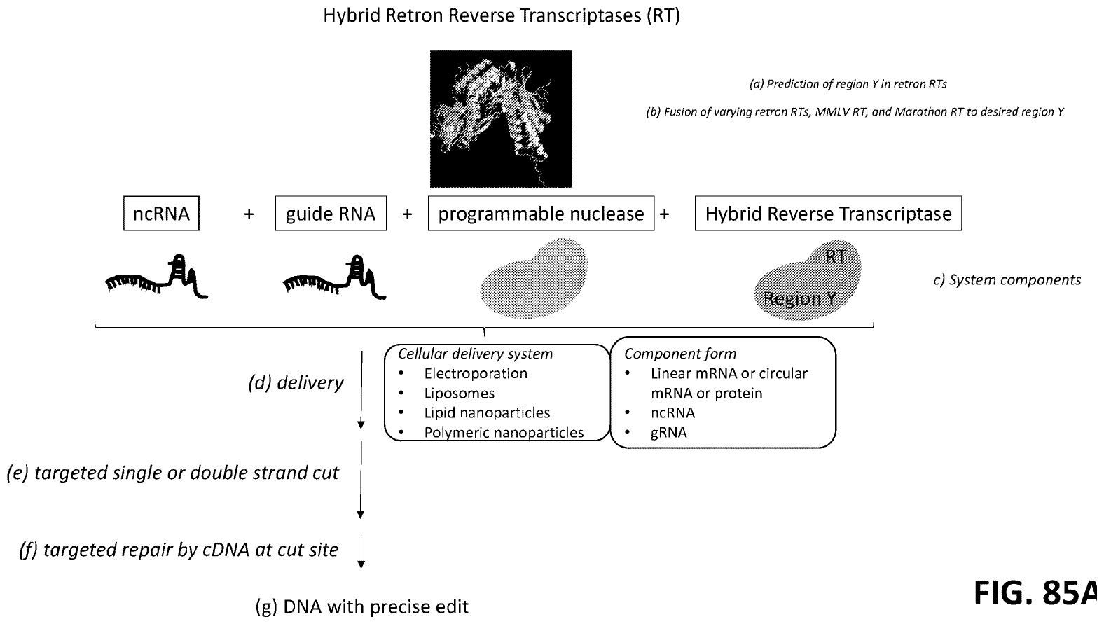

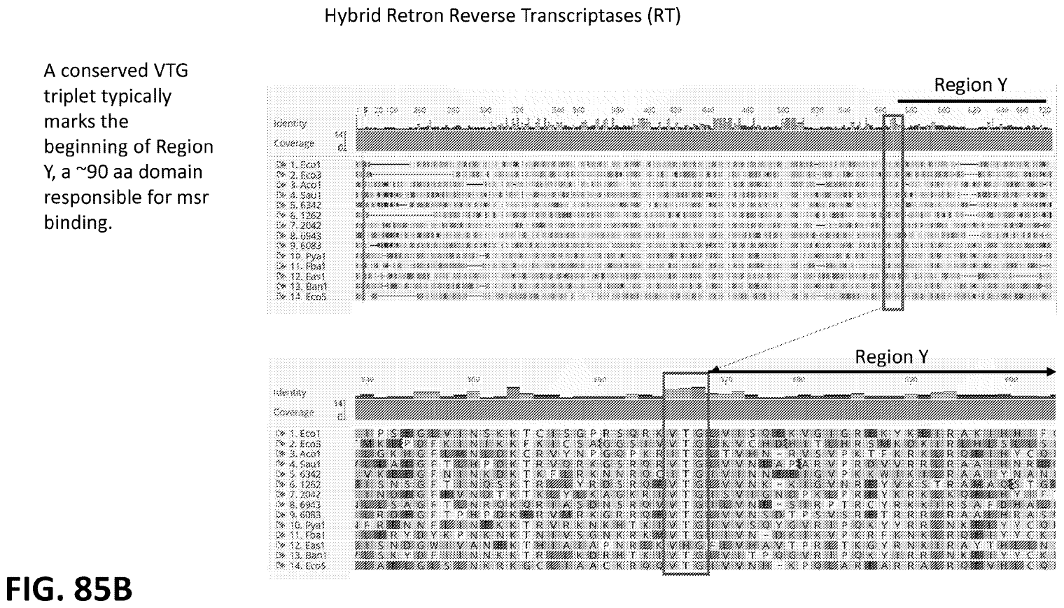

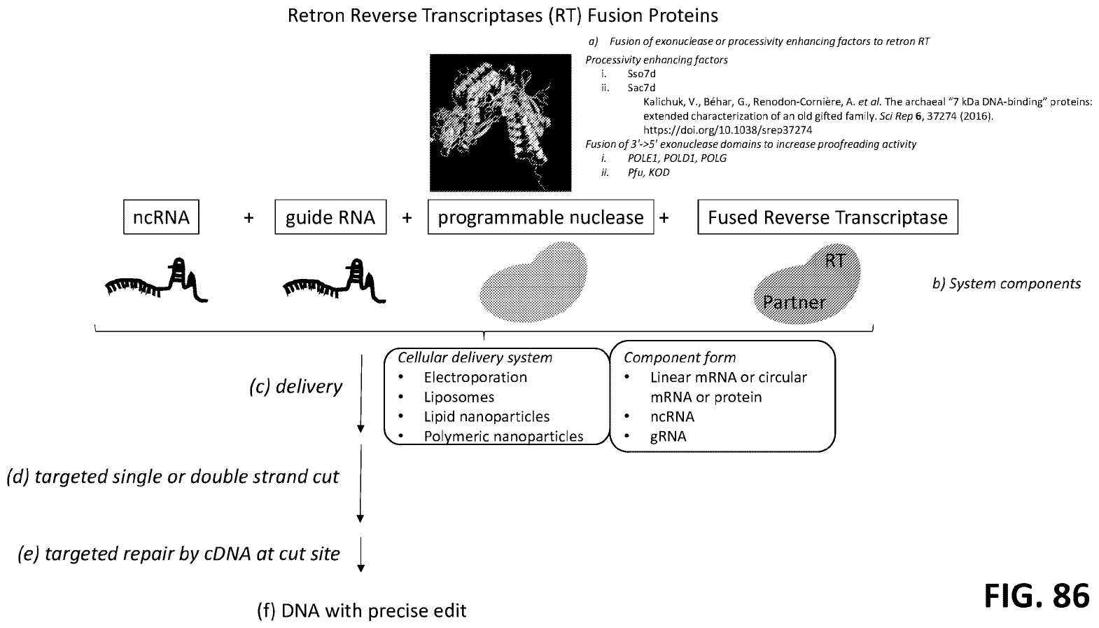

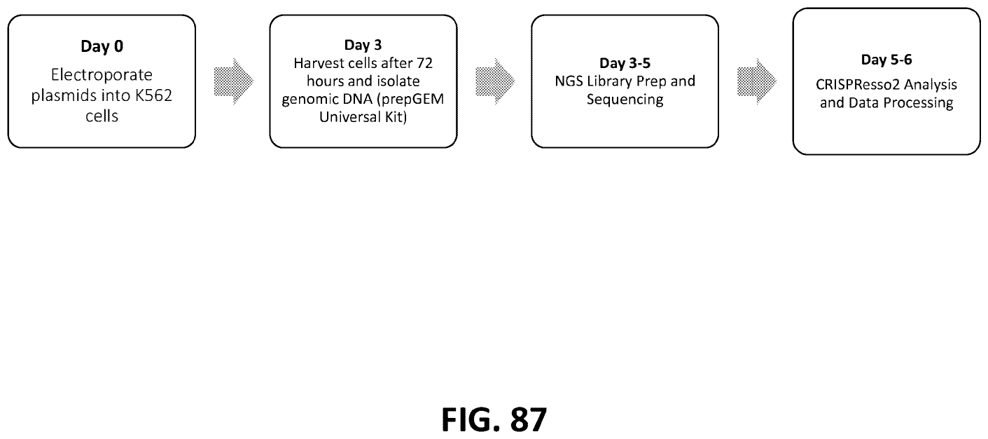

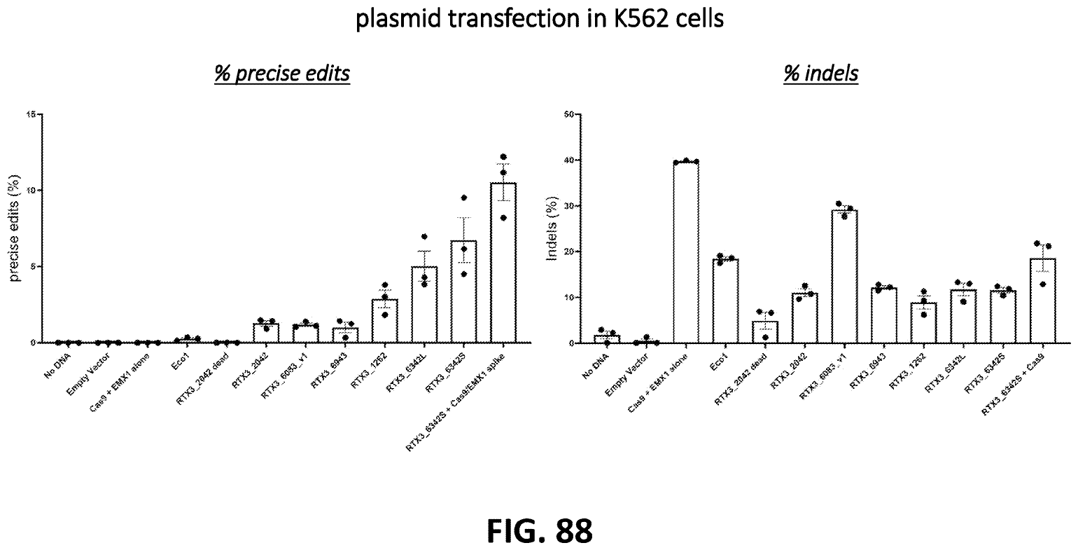

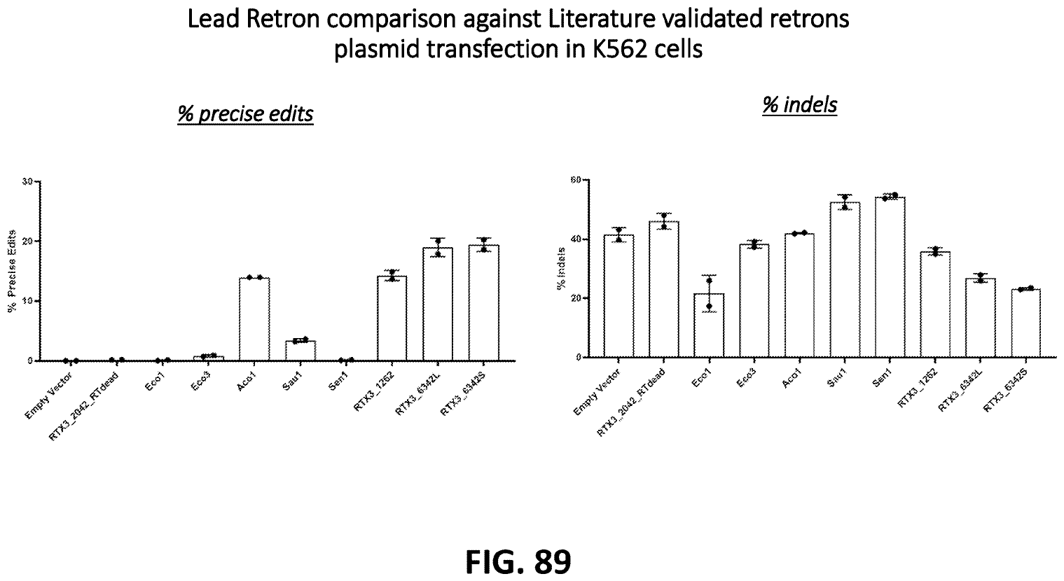

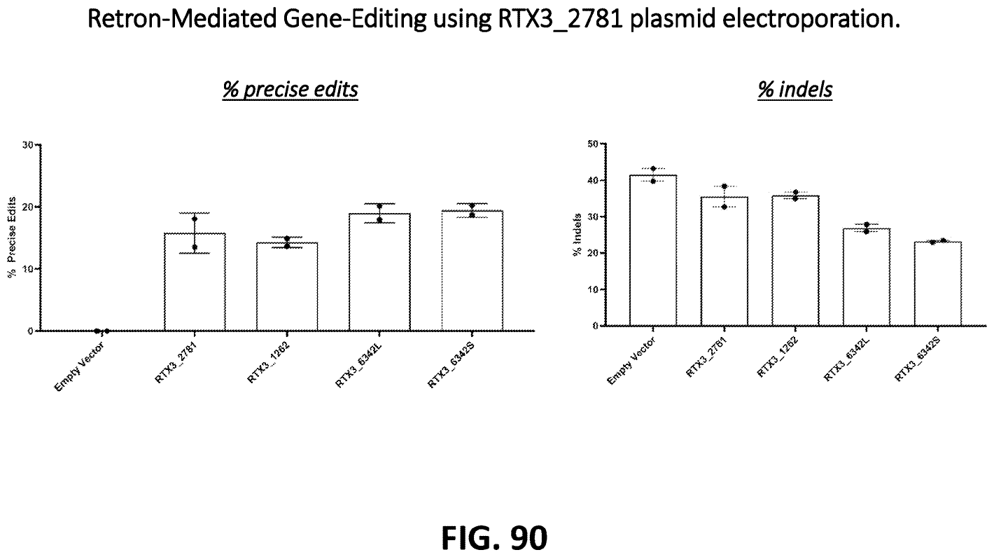

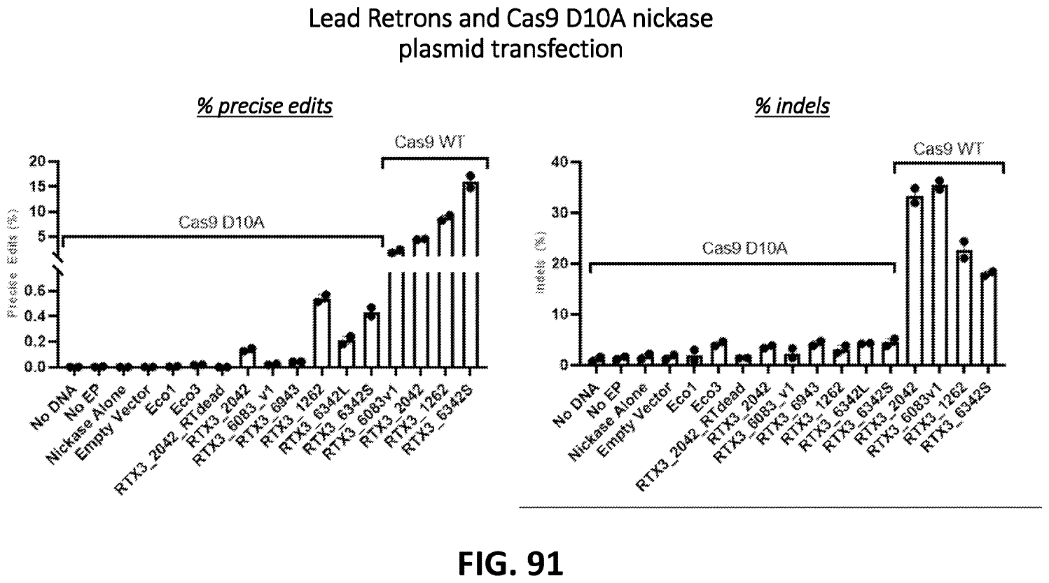

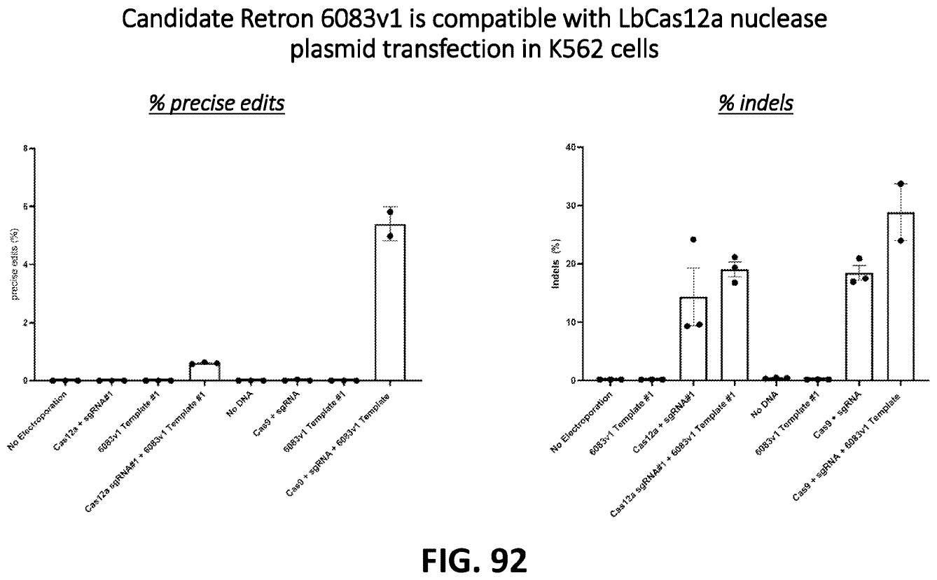

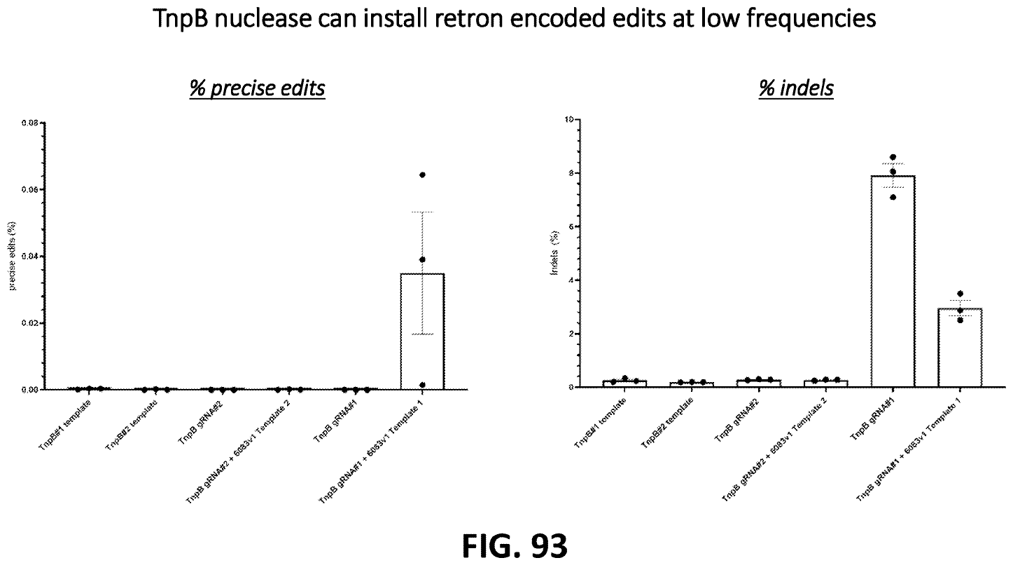

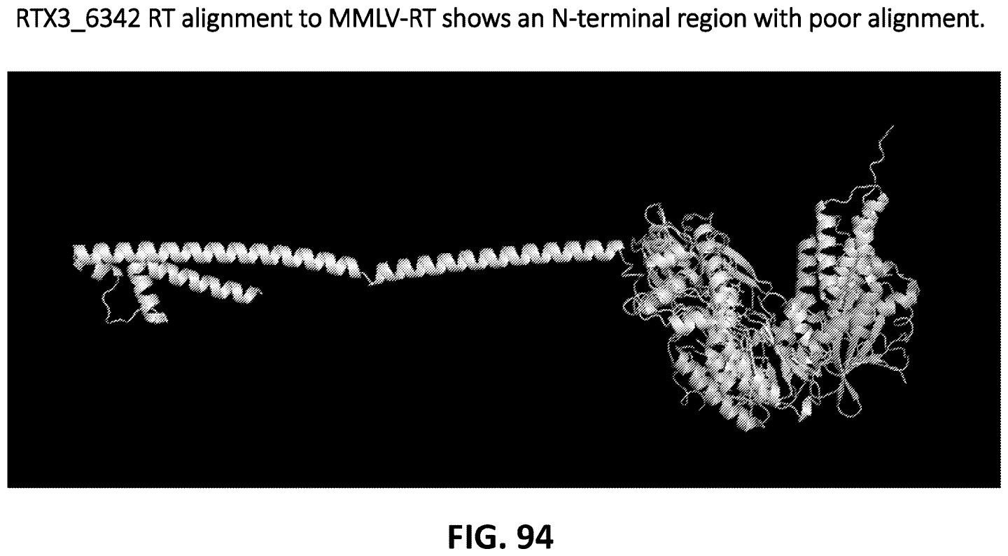

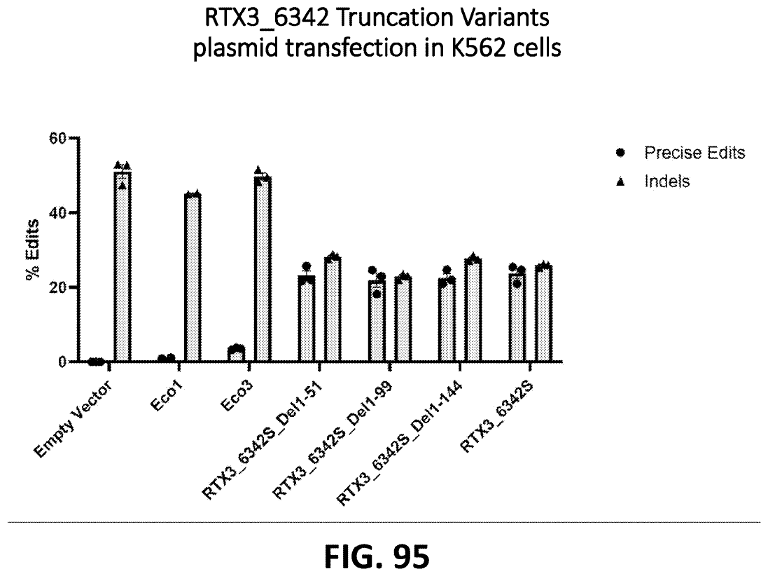

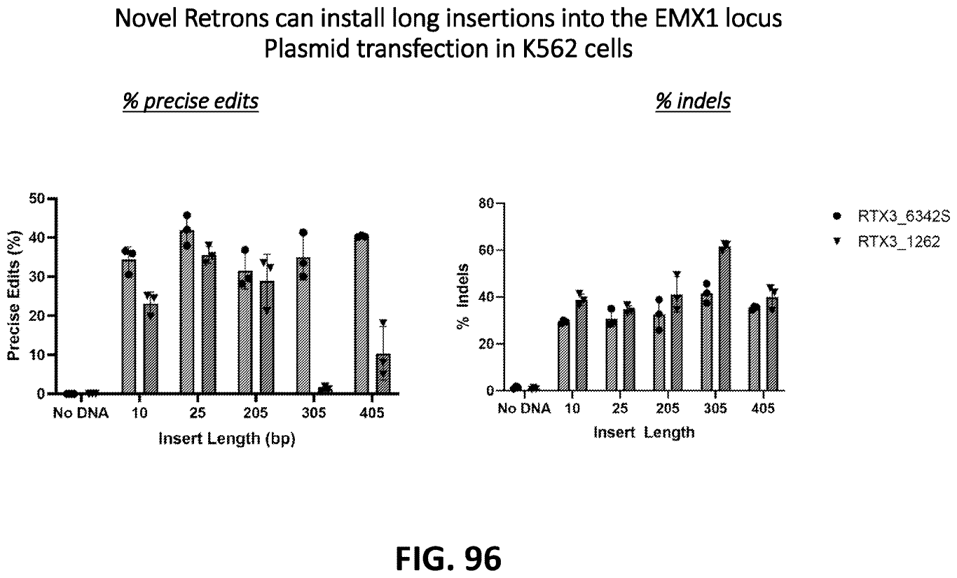

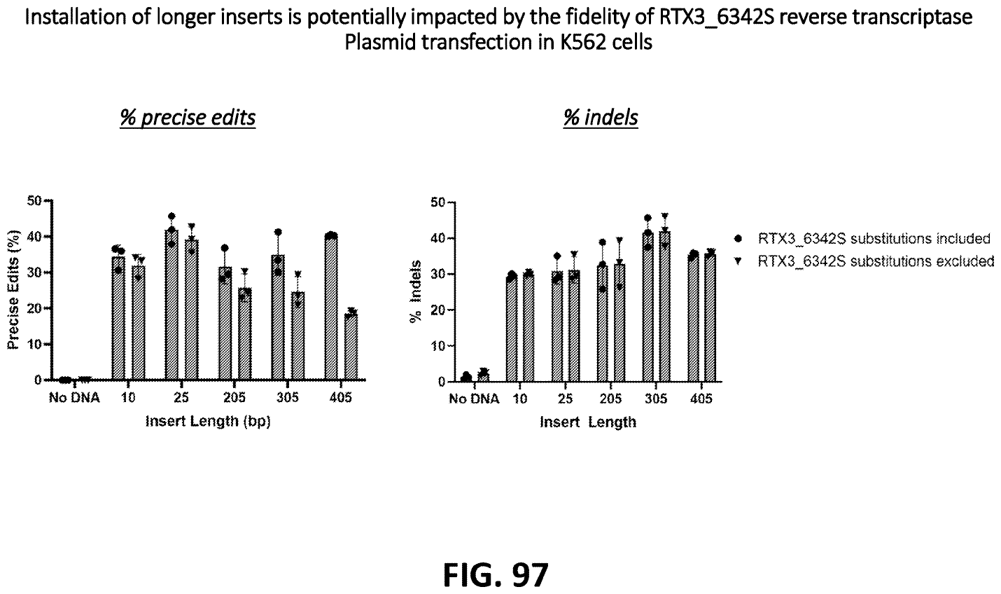

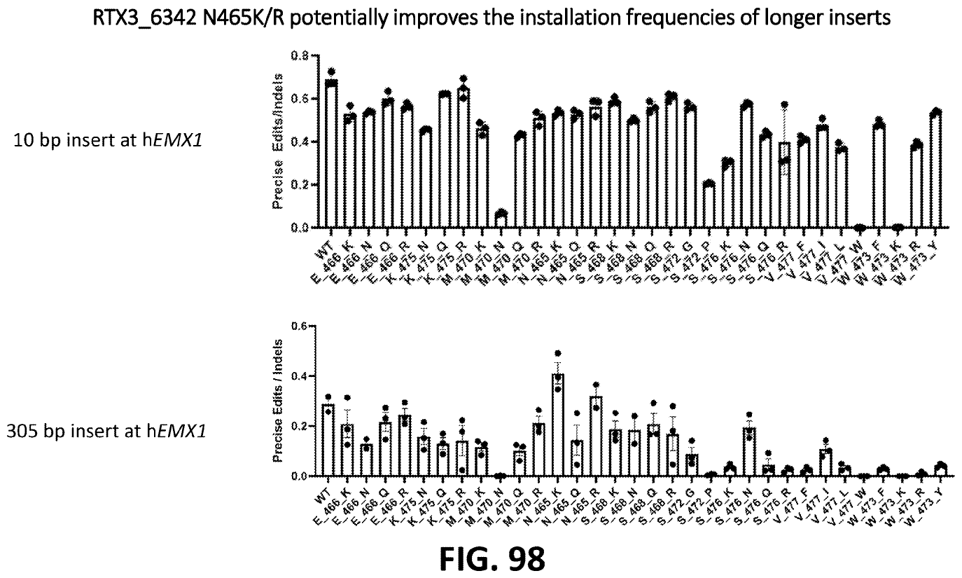

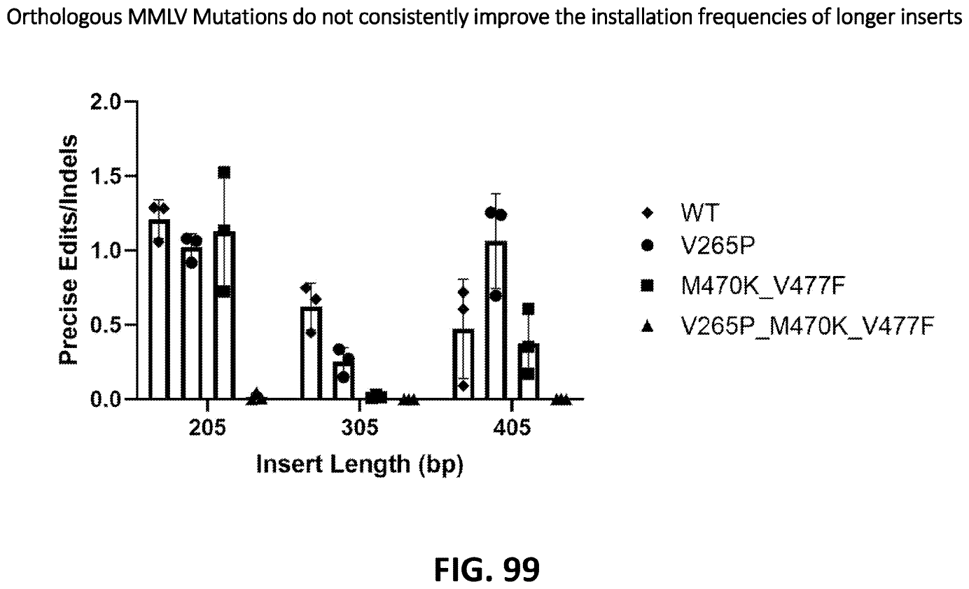

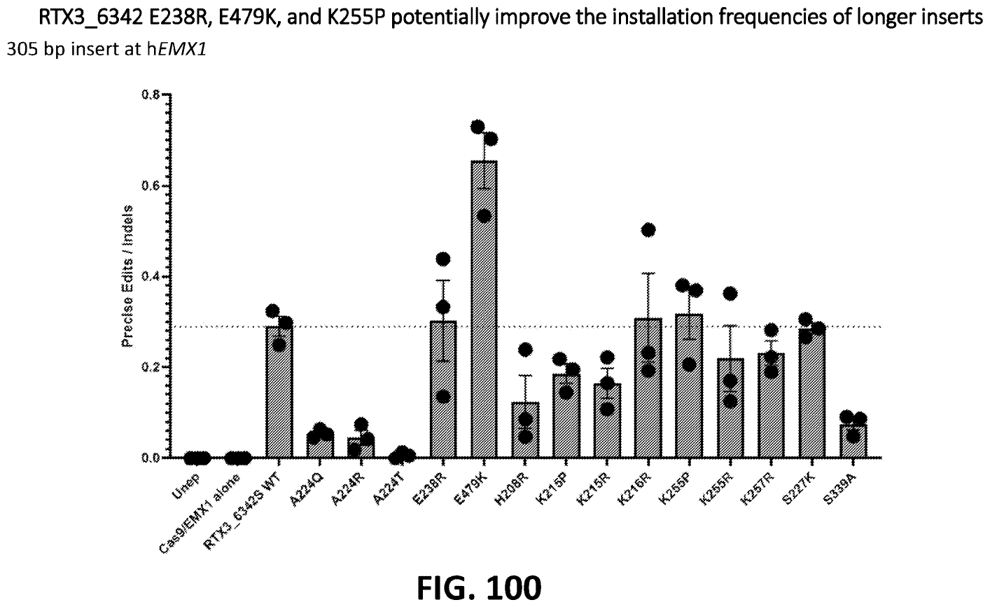

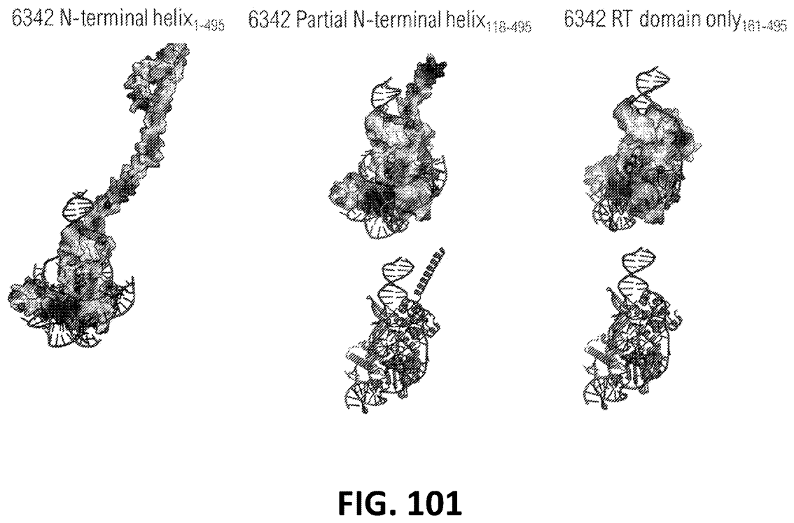

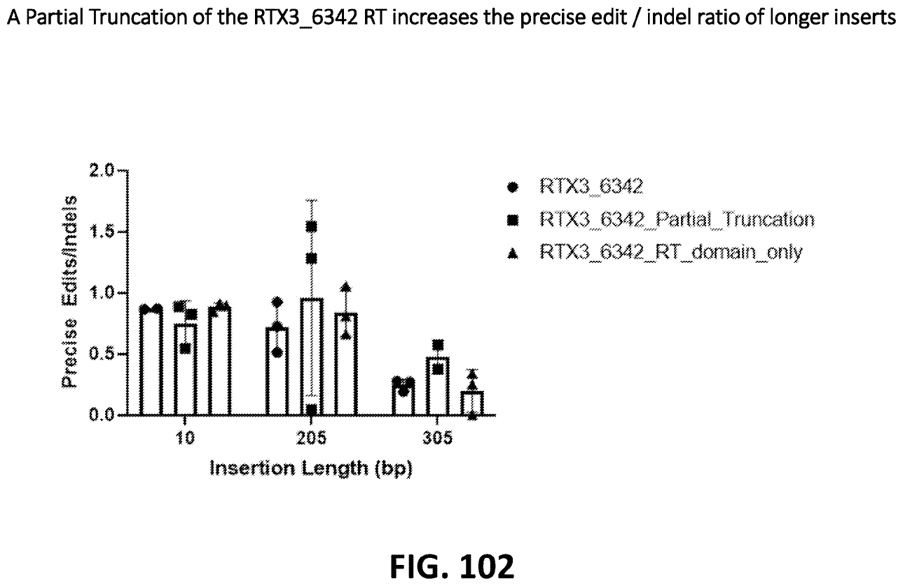

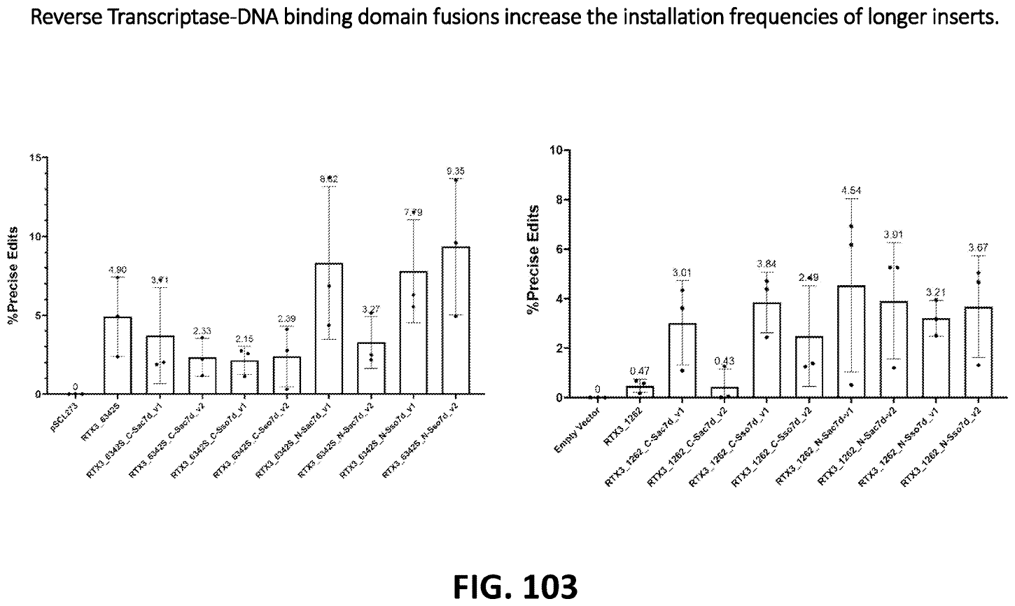

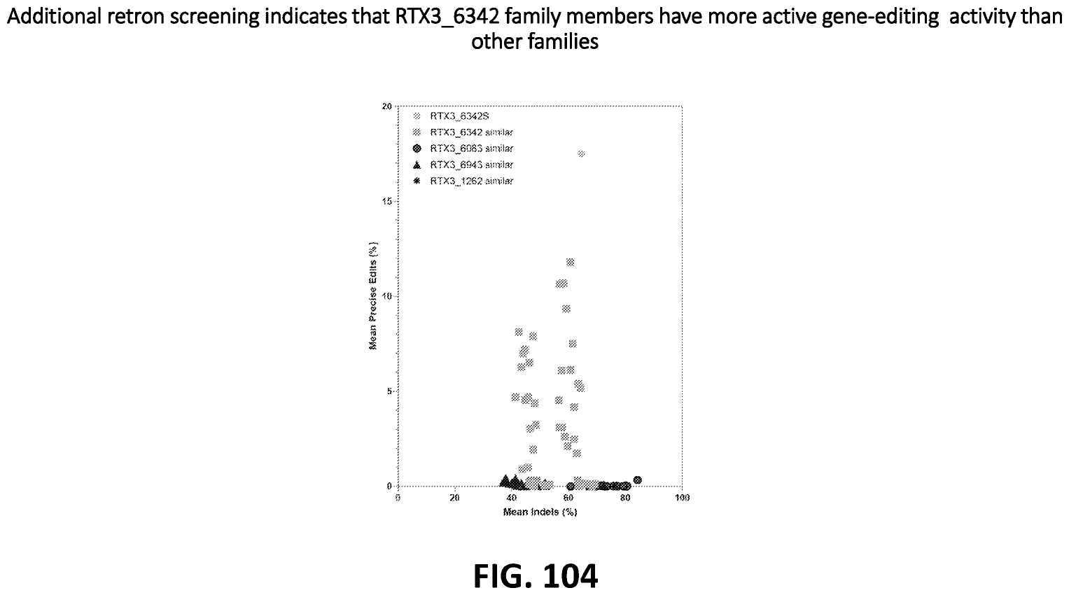

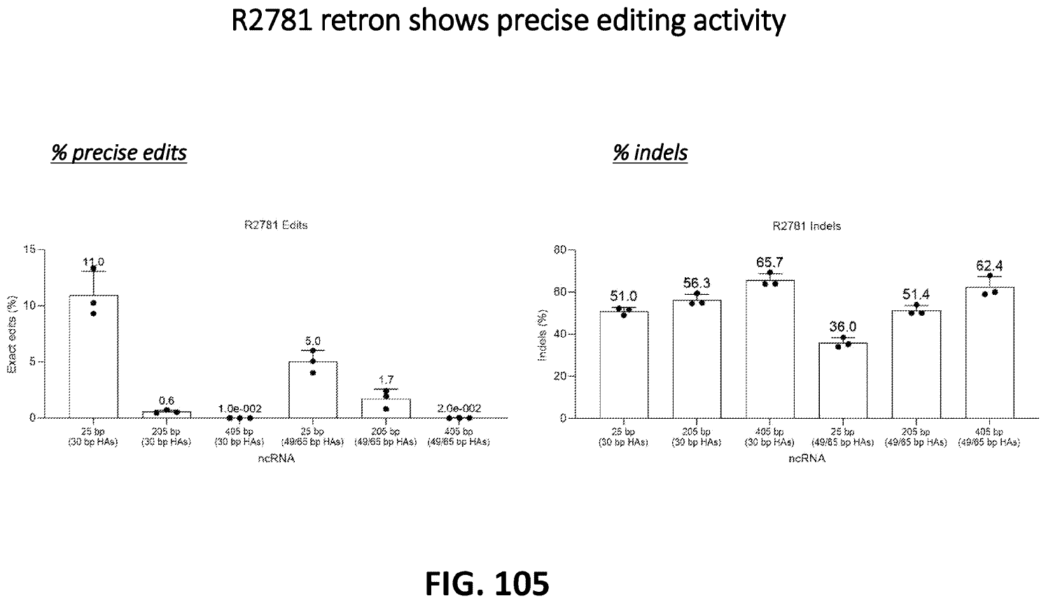

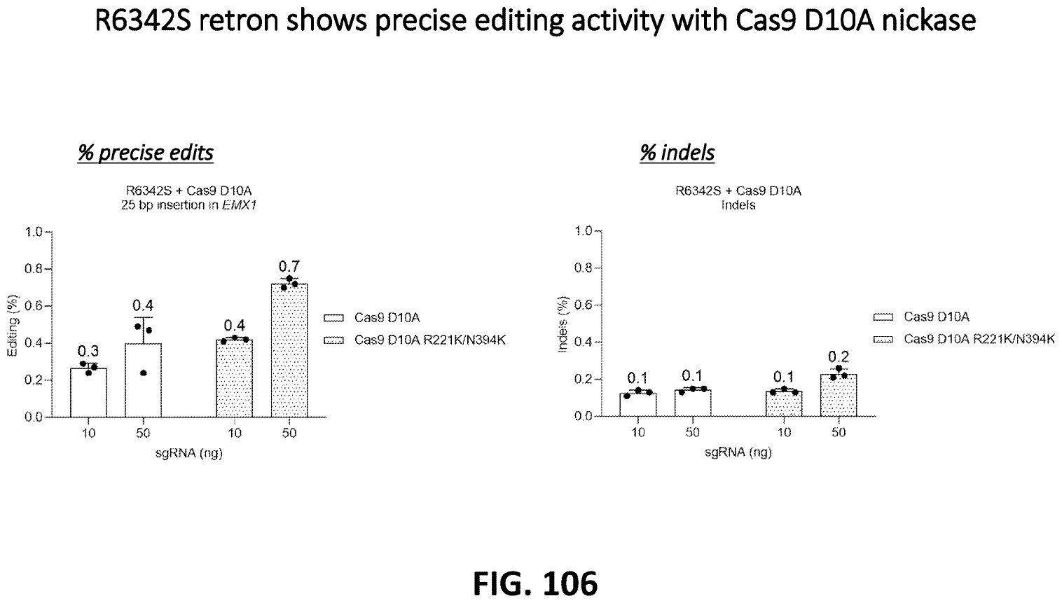

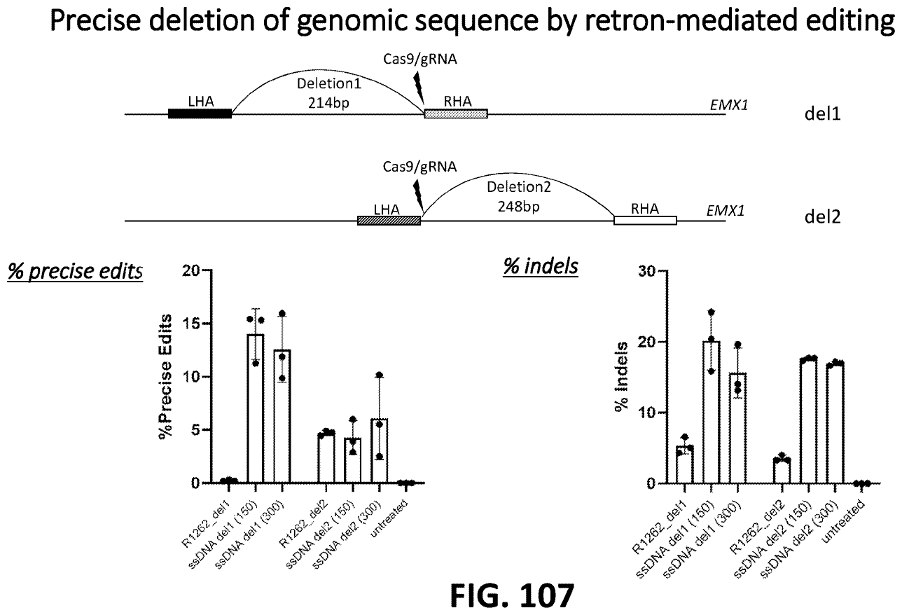

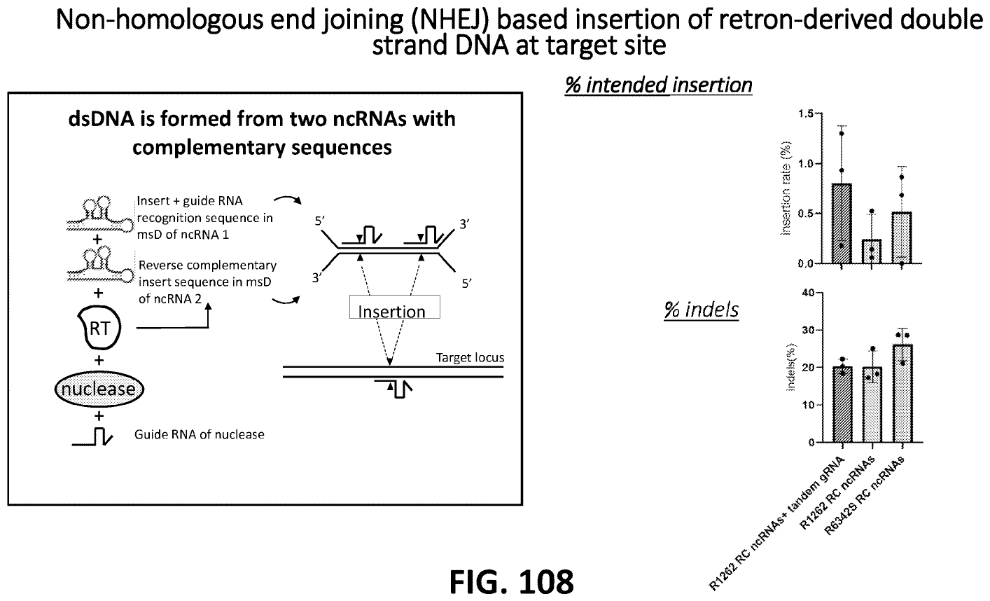

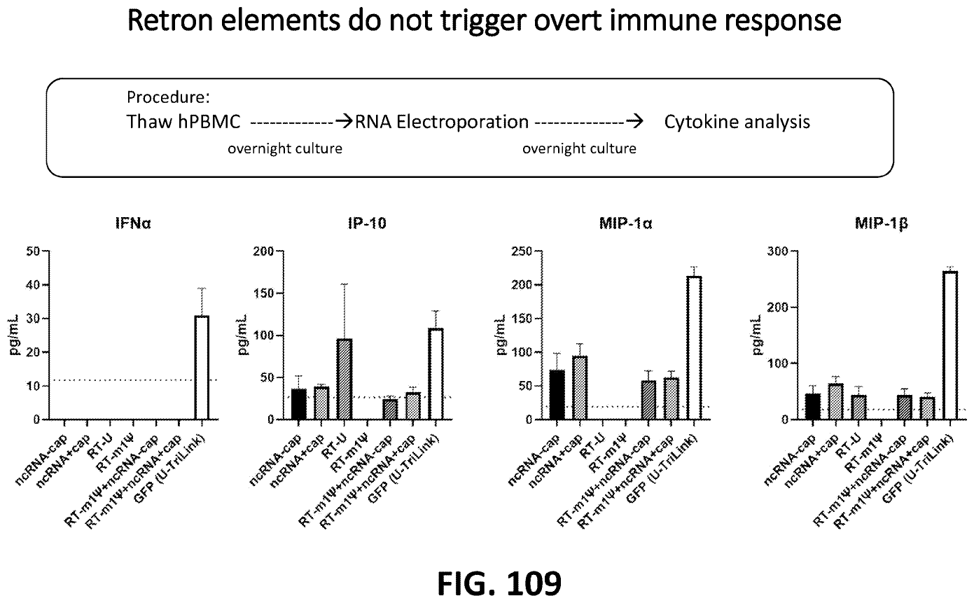

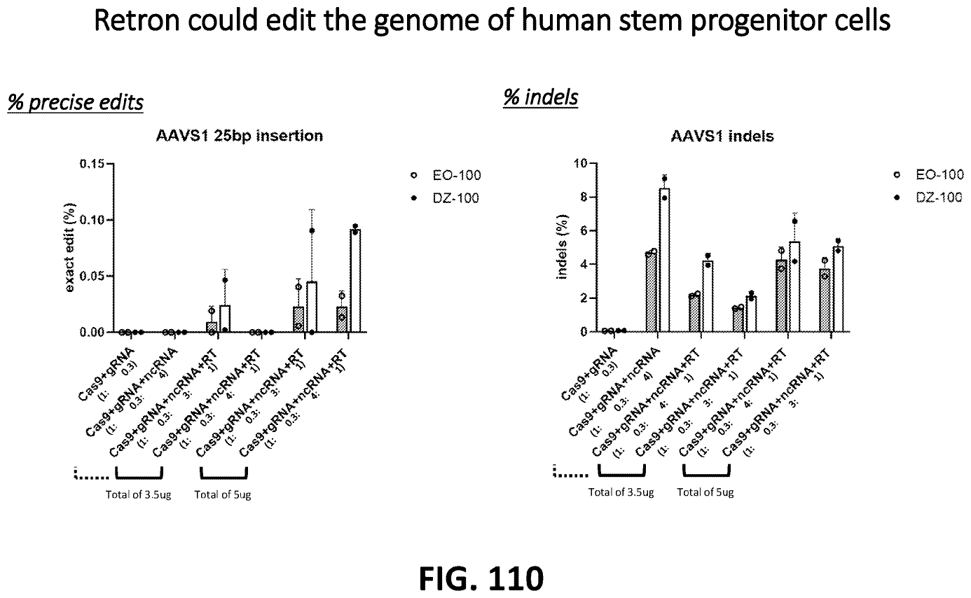

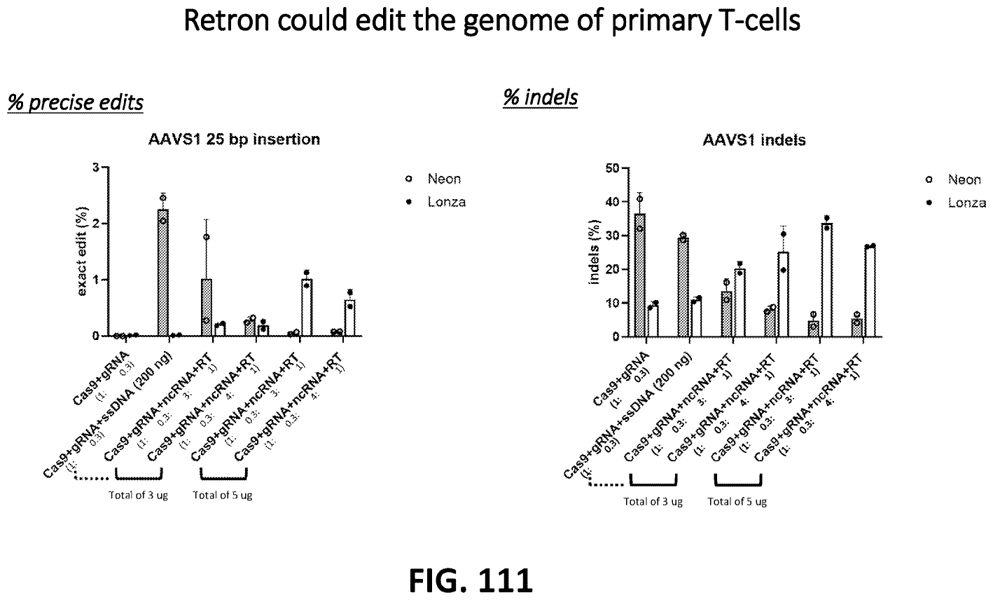

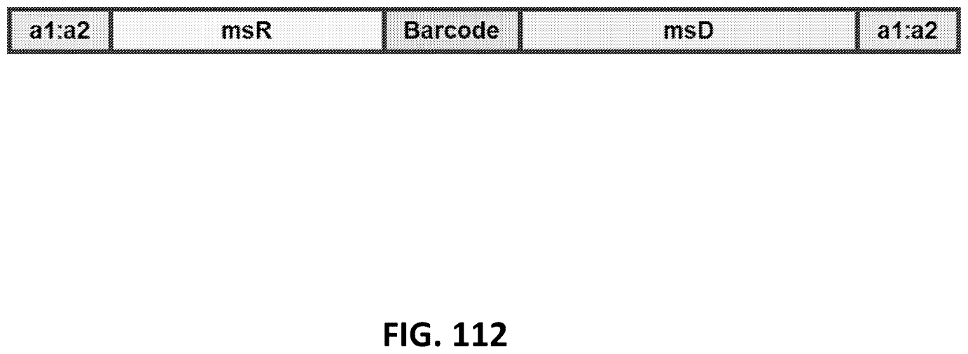

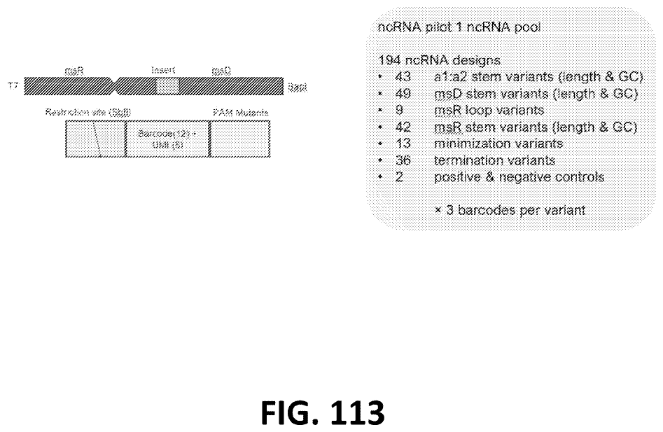

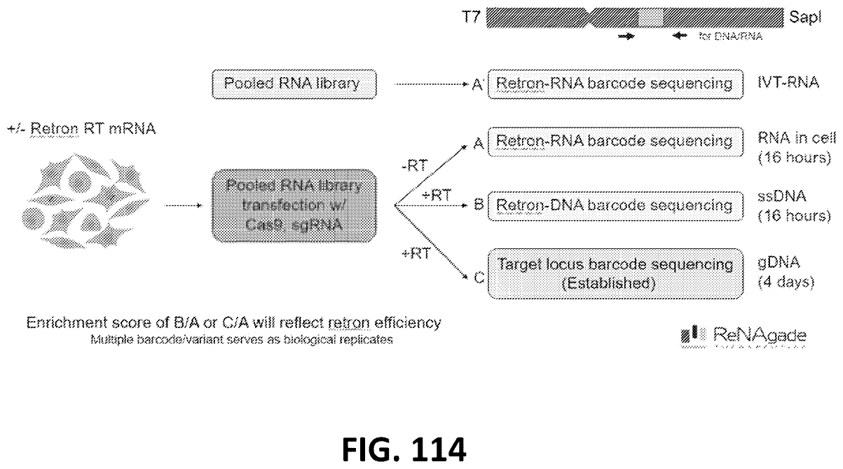

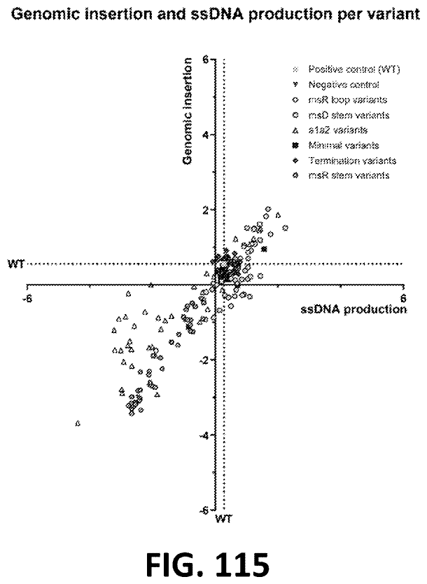

In certain embodiments, the engineered nucleic acid construct comprises: 1) an msr locus (that encodes the msr RNA portion of an msDNA); 2) an msd locus encoding the msd RNA portion of the msDNA; 3) a sequence encoding a retron reverse transcriptase (RT), wherein said msd RNA is capable of being reverse transcribed to form the msDNA by the retron reverse transcriptase (RT); and, 4) a heterologous nucleic acid inserted at or within the msd locus, upstream of the msr locus, upstream or downstream of the msd locus; wherein the engineered nucleic acid construct is engineered based on and/or to resemble a secondary structure of a wild-type or consensus retron encoding a wild-type or consensus retron ncRNA encompassed by: a) any one of the sequences and/or structures as depicted in any one of SEQ ID NOs: of Table B and/or FIGS. 2-27; or b) a variant of a), having: i) up to 1, 2, or 3 (e.g., up to 1) nucleotide changes per 10 red lettered-nucleotides; ii) up to 4, 5, or 6 (e.g., up to 1 or 2) nucleotide changes per 10 black lettered-nucleotides; and/or iii) up to 7, 8, or 9 (e.g., up to 3 or 4) nucleotide changes per 10 grey lettered-nucleotides; and/or optionally further comprising: i) 7, 8, 9, or 10 (e.g., 9 or 10) nucleotides present per 10 red-circled nucleotides; ii) 6, 7, 8, 9, or 10 (e.g., 8, 9 or 10) nucleotides present per 10 black-circled nucleotides; iii) 4, 5, 6, 7, 8, 9, or 10 (e.g., 6, 7, 8, 9 or 10) nucleotides present per 10 grey-circled nucleotides; and/or iv) 2, 3, 4, 5, 6, 7, 8, 9, or 10 (e.g., 4, 5, 6, 7, 8, 9, or 10) nucleotides present per 10 white-circled nucleotides; wherein the ncRNA does not comprise an ncRNA associated with the sequences of Table X.

The engineered nucleic acid construct (e.g., the engineered retron) may comprise one or more sequence modifications (e.g., an insertion, deletion, and/or substitution of one or more nucleotide(s)) in the msr locus and/or the msd locus that: a) modulates (e.g., enhances) reverse transcription, processivity, accuracy/fidelity, and/or production of the msDNA (e.g., in the mammalian cell); b) modulates (e.g., reduces) immunogenicity of ncRNA encoded by the engineered retron (e.g., the msr locus and/or the msd locus) in a host (e.g., a host comprising the mammalian cell); c) modulates (e.g., inhibits, either permanently or transiently) a function of the msDNA; and/or d) modulates (e.g., improves) efficiency of targeted genome editing/engineering.

In some embodiments, the engineered nucleic acid construct (e.g., the engineered retron) is engineered based on and/or to resemble a secondary structure of a wild-type or consensus retron encoding a wild-type or consensus retron ncRNA encompassed by: a) the sequence of any one of Table B ncRNA sequences and/or the structure depicted in any one of FIGS. 2-27; or b) a variant of a), having: i) up to 1, 2, or 3 (e.g., up to 1) nucleotide changes per 10 red lettered-nucleotides; ii) up to 4, 5, or 6 (e.g., up to 1 or 2) nucleotide changes per 10 black lettered-nucleotides; and/or iii) up to 7, 8, or 9 (e.g., up to 3 or 4) nucleotide changes per 10 grey lettered-nucleotides; and/or optionally further comprising: i) 7, 8, 9, or 10 (e.g., 9 or 10) nucleotides present per 10 red-circled nucleotides; ii) 6, 7, 8, 9, or 10 (e.g., 8, 9 or 10) nucleotides present per 10 black-circled nucleotides; iii) 4, 5, 6, 7, 8, 9, or 10 (e.g., 6, 7, 8, 9 or 10) nucleotides present per 10 grey-circled nucleotides; and/or iv) 2, 3, 4, 5, 6, 7, 8, 9, or 10 (e.g., 4, 5, 6, 7, 8, 9, or 10) nucleotides present per 10 white-circled nucleotides.

Another aspect of the disclosure provides a vector system comprising a vector comprising the engineered retron described herein.

Another aspect of the disclosure provides an isolated host cell comprising the engineered retron described herein, or the vector system described herein.

Another aspect of the disclosure provides a pharmaceutical composition comprising the engineered retron described herein, or the vector system described herein.

Another aspect of the disclosure provides a delivery vehicle comprising the engineered retron described herein or the ncRNA encoded by the engineered retron described herein, the vector or vector system described herein, the host cell described herein, or the pharmaceutical composition described herein.

Another aspect of the disclosure provides a kit comprising the engineered retron described herein or the ncRNA encoded by the engineered retron described herein, and optionally instructions for genetically modifying a cell using the engineered retron described herein or the ncRNA encoded by the engineered retron described herein.

Another aspect of the disclosure provides a method of modifying a target DNA sequence in a host cell (e.g., a mammalian cell), the method comprising introducing into the host cell (e.g., the mammalian cell) the engineered retron of the invention, the ncRNA encoded by the engineered retron of the invention, or the vector/vector system described herein, to allow the production of the msDNA in the host cell (e.g., mammalian cell), wherein at least a part of the heterologous nucleic acid in the msDNA is integrated into the genome of the host (e.g., mammalian) cell at the target DNA sequence. Optionally, the target sequence is recognized by a suitable nuclease, such as a CRISPR/Cas effector enzyme, a ZFN, a TALEN, a meganuclease, TnpB, IscB, or a restriction endonuclease (RE), and a double-stranded break (DSB) is created by the nuclease to facilitate/promote the insertion of the part of the heterologous nucleic acid into the target sequence. Further optionally, the target sequence modified/inserted by the part of the heterologous nucleic acid can no longer be recognized by the nuclease to re-create a DSB.

Another aspect of the disclosure provides a use of the engineered retron in the various methods described herein.

Another aspect of the disclosure provides a genome editing system comprising: a) nuclease capable of acting at a target site on a genome (e.g. human genome), such as a CRISPR/Cas effector enzyme, a ZFN, a TALEN, a meganuclease, TnpB, IscB, or a restriction endonuclease (RE); and b) an engineered retron described herein, or an ncRNA encoded thereby, or a vector or a vector system comprising or encoding the same. Optionally, the nuclease may be linked to one or more element(s) of the engineered retron or the encoded ncRNA. For example, in one embodiment, the nuclease may be linked (e.g., fused or conjugated) to the reverse transcriptase of the engineered retron described herein. In another embodiment, the nuclease may engage/bind to form a complex with a nucleic acid guide sequence (such as a single-guided RNA of a Cas enzyme), wherein the guide sequence is linked to the ncRNA and/or msDNA of the engineered retron described herein.

Another aspect of the disclosure provides an enhanced genome editing system, comprising the genome editing system of the disclosure connected to a biomolecule that modulates host DNA repair, in order to, for example, modulate (e.g., enhance) the incorporation of the heterologous nucleic acid sequence into a genome (e.g., human genome).

With the general aspect of the disclosure described herein, specific aspects and embodiments of the disclosure are further described in the sections below. It should be understood that any one embodiment of the disclosure, including those described only in the examples or the claims, or only in one section herein below, can be combined with any one or more additional embodiments of the invention, unless such combination is expressly disclaimed or are improper.

### A. Recombinant Retrons

The present disclosure provides engineered retrons, as well as compositions, systems, and methods that include or utilize the engineered retrons for genome modification, such as genome editing, cell recording, and recombineering.

Retrons were originally discovered in 1984 in Myxococcus xanthus bacterium when a short, multi-copy single-stranded DNA (msDNA) that is abundantly present in the bacterial cell was identified. Since then, a number of naturally existing retrons have been found in many prokaryotes such as bacteria.

As depicted in FIG. 1A, retrons encode and transcribe as a single RNA, which comprises a non-coding RNA (ncRNA) portion and a portion encoding a specialized reverse transcriptase (RT). The retron ncRNA (msr and msd) is the precursor of the hybrid molecule that eventually forms, and it initially folds into a typical RNA secondary structure that is recognized by the accompanying RT. The translated RT typically recognizes certain secondary structures in the ncRNA, and binds the RNA template downstream from the msd region. The RT initiates reverse transcription of the RNA towards its 5′ end, starting from the 2′-end of a conserved guanosine (G) residue found immediately after a double-stranded RNA structure (the a1/a2 region) within the ncRNA. A portion of the ncRNA serves as a template for reverse transcription, and reverse transcription terminates before reaching the msr locus. During reverse transcription, cellular RNase H degrades the segment of the ncRNA that serves as template, but not other parts of the ncRNA. The result of the reverse transcription, the msDNA, remains covalently attached to the RNA template via the 2′-5′ phosphodiester bond, and base-pairs with the RNA template using the 3′ end of the msDNA. See FIG. 1A for a general or typical organization of the retron coding sequence, including the RT encoding sequence and the msr and msd loci, as well as the synthesis of the msDNA by reverse transcription of the initial ncRNA transcript.

Many retrons also contain an accessory protein (not depicted in FIG. 1A), which may have a variable function that may not be fully understood. In certain embodiments, the engineered retrons described herein do not comprise the accessory protein naturally associated with the wild-type or template retron.

Applicant has discovered, analyzed, and phylogenetically classified 7257 previously unknown retrons from nature based on multiple criteria, including sequence homology and conserved predicted secondary structures, and has grouped these retrons into different phylogenetic clades based on sequence homology and/or conserved predicted secondary structures. These clades include Type IA_IIA1 (FIG. 2), Type 1B1 (FIG. 3), Type IB2 (FIG. 4), Type 1C (FIG. 5), Type IIA1 other (FIG. 6), Type IIA2 (FIG. 7), Type IIA3 (FIG. 8), Type IIA4 (FIG. 9), Type IIA5 (FIG. 10), Type IIIA1 (FIG. 11), Type IIIA2 (FIG. 12), Type IIIA3 (FIG. 13), Type IIIA4 (FIG. 14), Type IIIA5 (FIG. 15), Type IIIunk (FIG. 16), Type IV (FIG. 1X), Type V (FIG. 19), Type VI (FIG. 20), Type XI Group 1 (FIG. 21), Type XI (Group 2) (FIG. 22), Type XII (FIG. 23), Type XIII (FIG. 24), Type XIV (FIG. 25), Eco107-like (FIG. 26), and Outgroup A (FIG. 27). The disclosure further describes the engineering and/or modification of these newly discovered retron sequences as a starting point to obtain useful recombinant retrons, such as those depicted in FIG. 1B.

FIG. 1B.1 depicts an embodiment of a recombinant retron construct (e.g., a nucleotide sequence cloned into an expression vector) contemplated by the present disclosure. In the top left schematic, the single thin black line represents a double-stranded nucleotide sequence (e.g., as cloned into an expression vector, such as a plasmid). The recombinant retron is constructed by modifying a starting point retron DNA sequence encoding a ncRNA (the msr/msd region) (such as any one of the herein disclosed 7257 newly discovered retron sequences, and specifically any one of the 7257 ncRNA sequences of Table B. A starting point retron DNA sequence encoding an ncRNA may be modified in any number of ways and can including one modification or more than one modification. For example, the retron DNA may modified to contain at least one nucleotide modification, including a single nucleotide substitution, insertion, or deletion, or a substitution, insertion, or deletion of more than one nucleotide, i.e., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or up to 100, or up to 200, 300, 400, 500, 600, 700, 800, 900, 1000, 1100, 1200, 1300, 1400, 1500, 1600, 1700, 1800, 1900, or up to 2000 nucleotides substituted, inserted, or deleted from a starting point retron (e.g., a wildtype retron). Where more than one nucleotide of a starting point retron (e.g., a wildtype retron) is substituted, deleted, or inserted, the nucleotides may be contiguous or non-contiguous. While an engineered retron as a whole is not naturally-occurring, it may include components such as nucleotide sequences that do occur in nature. For example, an engineered retron can have nucleotide sequences from different organisms (e.g., from different bacteria species), or from completely synthetic/artificial/recombinant nucleic acid sequences. Thus, an engineered retron can have a bacterial nucleotide sequence, a human nucleotide sequence, a viral nucleotide sequence, and/or a synthetic/artificial/recombinant nucleotide sequence, and/or combinations of such sequences. An example of modifications of the recombinant retrons disclosed herein include the insertion of a heterologous nucleic acid sequence in a retron, for example, inserted into the ncRNA locus, such as in the msr or the msd loci. Linking guide RNA molecules to the 5′ and/or 3′ ends (i.e., linking one at the 5′ end of a ncRNA and/or one at the 3′ end of a ncRNA) also represents another modification contemplated by the recombinant retrons disclosed herein. In such embodiments, the guide RNA molecules may also be categorized or referred to more generally as types of heterologous nucleic acid sequences used to modify starting point retrons. These modifications are depicted in FIG. 1B.

In addition to the DNA encoding the ncRNA, the DNA encoding the RT may also be modified to obtain a recombinant RT. For example, the RT-encoding DNA may modified to contain at least one nucleotide modification, including a single nucleotide substitution, insertion, or deletion, or a substitution, insertion, or deletion of more than one nucleotide, i.e., up to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or up to 100, or up to 200, 300, 400, 500, 600, 700, 800, 900, 1000, 1100, 1200, 1300, 1400, 1500, 1600, 1700, 1800, 1900, or up to 2000 nucleotides substituted, inserted, or deleted from a starting point retron (e.g., a wildtype retron) within the RT gene.

Such modifications to the DNA encoding ncRNA and/or RT may modulate the function of the ncRNA and/or RT in various ways, including i) modulating (e.g., enhancing) reverse transcription, processivity, accuracy/fidelity, and/or production of the msDNA (e.g., in the mammalian cell); ii) modulating (e.g., reducing) immunogenicity of ncRNA (msr locus and msd locus) encoded by the engineered retron in a host (e.g., a host comprising the mammalian cell); iii) modulating (e.g., inhibits, either permanently or transiently) a function of the msDNA; and/or iv) modulating (e.g., improving) efficiency of targeted genome editing/engineering.

In one embodiment, the present disclosure provides recombinant retrons having the general structure of: a) an msr locus; b) an msd locus encoding the msd RNA portion of the msDNA; c) a sequence encoding a retron reverse transcriptase (RT) (optionally in trans to the ncRNA), wherein the msd RNA is capable of being reverse transcribed (e.g., in a host cell such as a mammalian cell) to form an msDNA by the retron reverse transcriptase (RT); d) a heterologous nucleic acid (e.g., heterologous DNA) capable of being transcribed with the msr locus and/or the msd locus, optionally, the heterologous nucleic acid is inserted at or within the msd locus, upstream of the msr locus, upstream or downstream of the msd locus.

The engineered retrons of the invention are optionally structurally further modified to include one or more heterologous nucleic acids. The engineered retron may be further modified to provide various functional improvements, such as (without limitation), to enhance the production of msDNA in a cell (e.g., a mammalian cell, including a human cell).

In certain embodiments, the disclosure provides engineered retrons based on their conserved predicted secondary structures, such as those in FIGS. 2-27.

In other embodiments, the disclosure provides engineered retrons based on their sequence identity. Exemplary RT amino acid sequences and ret gene nucleic acid sequences are provided in Table A. Exemplary RT consensus amino acid sequences and/or ret gene nucleic acid sequences are provided in Table C. Exemplary ncRNA sequences are provided in Table B.

Retron sequences that may be provisoed out of the scope of the invention in some embodiments are provided in Table X.

In certain embodiments, exemplary engineered retrons of the invention (1) are engineered based on or engineered to resemble the secondary structures as depicted in any one of FIGS. 2-27, and/or (2) are provided in Table B. Sequences with significant sequence percentage identity (e.g., at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity) are also within the scope of the invention.

In certain embodiments, the engineered nucleic acid construct comprises: 1) an msr locus (that encodes the msr RNA portion of an msDNA); 2) an msd locus encoding the msd RNA portion of the msDNA; 3) a sequence encoding a retron reverse transcriptase (RT), wherein said msd RNA is capable of being reverse transcribed to form the msDNA by the retron reverse transcriptase (RT); and, 4) a heterologous nucleic acid inserted at or within the msd locus, upstream of the msr locus, upstream or downstream of the msd locus; wherein the engineered nucleic acid construct is engineered based on and/or to resemble a secondary structure of a wild-type or consensus retron encoding a wild-type or consensus retron ncRNA encompassed by: a) any one of the sequences and/or structures as depicted in any one of SEQ ID Nos of Table B and/or FIGS. 2-27; or b) a variant of a), having: i) up to 1, 2, or 3 (e.g., up to 1) nucleotide changes per 10 red lettered-nucleotides; ii) up to 4, 5, or 6 (e.g., up to 1 or 2) nucleotide changes per 10 black lettered-nucleotides; and/or iii) up to 7, 8, or 9 (e.g., up to 3 or 4) nucleotide changes per 10 grey lettered-nucleotides; and/or optionally further comprising: i) 7, 8, 9, or 10 (e.g., 9 or 10) nucleotides present per 10 red-circled nucleotides; ii) 6, 7, 8, 9, or 10 (e.g., 8, 9 or 10) nucleotides present per 10 black-circled nucleotides; iii) 4, 5, 6, 7, 8, 9, or 10 (e.g., 6, 7, 8, 9 or 10) nucleotides present per 10 grey-circled nucleotides; and/or iv) 2, 3, 4, 5, 6, 7, 8, 9, or 10 (e.g., 4, 5, 6, 7, 8, 9, or 10) nucleotides present per 10 white-circled nucleotides; wherein the ncRNA does not comprise an ncRNA associated with the sequences of Table X.

In certain embodiments, insertion of a heterologous nucleic acid includes deletion of a retron nucleic acid. In certain embodiment, the inserted heterologous nucleic acid substitutes for a retron nucleic acid which is deleted. There is no requirement that the inserted heterologous nucleic acid and the deleted retron nucleic acid be of the same or similar size. In certain embodiments, substitution comprises replacement of a portion of a hairpin loop region of a retron. In certain embodiments, substitution comprises replacement of a portion of a stem-loop region of a retron. In certain embodiments, substitution comprises replacement of a region of a retron that does not comprise a stem or a loop. In certain instances, identification of retron structure involves comparison to model ncRNA structures provided herein, e.g. by comparison to one or more of FIGS. 2-27. In certain instances, identification of retron structure involves modeling by an nucleic acid folding algorithm, such as but not limited to RNAfold (Gruber A R, et al., The Vienna RNA Websuite. Nucleic Acids Research, Volume 36, Issue suppl_2, 1 Jul. 2008, Pages W70-W74), UNAFold (Markham N R, et al., UNAFold: software for nucleic acid folding and hybridization. Methods Mol Biol. Vol. 453, 2008, Pages 3-31), and SPOT-RNA (Singh J et al., RNA secondary structure prediction using an ensemble of two-dimensional deep neural networks and transfer learning. Nature communications, Vol. 10, 2019, Pages 1-13).

In certain embodiments, the engineered retron of the invention encodes a reverse transcriptase (RT) or a functional domain thereof, comprising: i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A. In some embodiments, the RT does not comprise a polypeptide identified in Table X.

In certain embodiments, the engineered retron of the invention encodes a reverse transcriptase (RT) or a functional domain thereof, comprising: i) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide of Table A and/or ii) a consensus polynucleotide sequence listed in Table A. In some embodiments, the polynucleotide encoding the RT does not comprise a polynucleotide identified in Table X.

In certain embodiments, the engineered retron of the invention encodes an ncRNA comprising: (I) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA of Table B.

Engineered ncRNAs of the invention can diverge in size from ncRNAs on which they are based and the proportion of the ncRNA retained in the engineered ncRNA can vary. In certain embodiments the retained amount of the ncRNA is about 50%, about 55%, about 60%, about 65%, about 70%, about 75%, about 80%, about 85%, about 88%, about 90%, about 91%, about 92%, about 93%, about 94%, about 95%, about 96%, about 97%, about 98%, about 99%, about 99.5%, or from 50% to 80%, or from 60% to 85%, of from 70% to 90%, or from 80% to 95%, or from 85% to 98%, or from 90% to 99%, or all of the ncRNA.

In certain embodiments, the engineered retron of the invention encodes an ncRNA and a reverse transcriptase (RT) or a functional domain thereof, wherein the ncRNA and the RT or functional domain thereof are as described above.

Specifically, in such embodiments, the ncRNA may comprise: (I) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B; and wherein the ncRNA optionally excludes the ncRNA associated with the sequences identified in Table X.

Also in such embodiment, the reverse transcriptase (RT) or functional domain thereof comprises: (A) i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or ii) a polypeptide listed in Table C; optionally, the RT does not comprise a polypeptide identified in Table X; OR (B) i) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.10%, at least 99.2%, at least 99.3%, at least 99.4%, at least 99.5%, at least 99.6%, at least 99.7%, at least 99.8% or at least 99.9% sequence identity to a polynucleotide listed in Table A; optionally, the polynucleotide encoding the RT does not comprise a polynucleotide associated with the sequences identified in Table X.

In certain embodiments, the heterologous nucleic acid is between >20 nucleotides and about 10,000 nucleotides.

The engineered retron may further comprise a sequence modification (e.g., insertion, deletion, and/or substitution of one or more nucleotide(s)) in the msr locus and/or the msd locus that: i) modulates (e.g., enhances) reverse transcription, processivity, accuracy/fidelity, and/or production of the msDNA (e.g., in the mammalian cell); ii) modulates (e.g., reduces) immunogenicity of ncRNA (msr locus and msd locus) encoded by the engineered retron in a host (e.g., a host comprising the mammalian cell); iii) comprises a nucleotide sequence that modulates (e.g., inhibits, either permanently or transiently) a function of the msDNA; and/or iv) modulates (e.g., improves) efficiency of targeted genome editing/engineering.

Retron msr gene, msd gene, and RT nucleic acid sequences (e.g., the ret gene) as well as the encoded retron reverse transcriptase protein sequences that may serve as the template of the engineered retron described herein may be derived from any source, such as those in Table A, optionally excluding those associated with the sequences of Table X.

In some embodiments, template or wild-type (wt) sequences of the msr gene, msd gene, and the RT coding sequence (viz., the ret gene) used in the engineered retron are derived from a bacterial retron.

In some embodiments, representative template/wild-type retrons are from gram negative bacteria. In some embodiments, the retron is from a bacterium listed in Table X.

In some embodiments, the engineered retrons are engineered based on clades defined on retron/retron RTs, in which the retrons are associated with a tripartite system composed of the ncRNA, the RT and an additional protein or RT-fused domain with diverse enzymatic functions. See, for example, “Mestre et al., Systematic Prediction of Genes Functionally Associated with Bacterial Retrons and Classification of The Encoded Tripartite Systems, Nucleic Acids Research, Volume 48, Issue 22, 16 Dec. 2020, Pages 12632-12647” (incorporated herein by reference). While the clades are based primarily upon naturally occurring ncRNA and retron/retron RT, and an additional protein or RT-fused domain, the clades, for the purpose of serving as the templates for the subject engineered retrons, are not limited to naturally occurring sequences. Rather, the clades can also encompass non-naturally occurring ncRNA and RT, including, without limitation, recombinant, modified or altered, chimeric, hybrid, synthetic, artificial, etc.

Thus, according to the instant disclosure, retrons may be considered phylogenetically related based on a Neighbor-Joining algorithm of at least 75% (of at least 1000 replicates) and a Poisson correction distance measurement of no more than 0.05, based on alignment of the retron RT. Alternatively or in addition, retrons may be considered phylogenetically related when/if the same RT, or closely related RT, can recognize the secondary structures of the ncRNA of the retrons and reserve transcribe the retrons to produce msDNA.

In certain embodiments, sequence alignments between different retron sequences (e.g., ncRNA and/or RT (protein and/or nucleic acid) sequences) or secondary structure generations are based on software known to one of ordinary skill in the art.

The retron ncRNA sequences including msr and msd sequences within the same clade may be highly conserved at certain positions, while being less conserved at other positions.

Exemplary consensus sequences based on clade members were generated (see, for example, the corresponding FIGS. 2-27, respectively) to show these conserved sequences and/or secondary structures, including the highly conserved nucleotides with at least 97% sequence conservation at red lettered-nucleotides, those with between 90-97% sequence conservation at black lettered-nucleotides, and 75-90% nucleotide sequence identity at grey-lettered nucleotides. Further structural limitations of the consensus sequences for the clades are provided as colored circles indicating the probability of having a base at that specific position, including red circles representing a base in 97% of the cases, black circles representing a base in 90-97% of the cases, and grey circles representing a base in 75-90% of the cases.

In some embodiments, the template ncRNA based on which the subject engineered retron is modified (including the msr and msd region sequences) is a consensus sequence for the various retron ncRNA (including msr and msd nucleic acid sequences) clades, as provided in any one of SEQ ID NOs: of Table B and the corresponding FIGS. 2-27, respectively, including the bases that are highly conserved and depicted by a specific-colored letters or circles, and optionally further including bases that may be present at specific locations by specific-colored circles.

In some embodiments, the engineered retron is engineered, based on and/or to resemble a secondary structure of a wild-type or consensus retron encoding a wild-type or consensus retron ncRNA encompassed by: 1) the sequences and/or structures as depicted in any one of SEQ ID NOs: of Table B and the corresponding FIGS. 2-27, respectively; or 2) a variant of 1), having: A) up to 1, 2, or 3 (e.g., up to 1) nucleotide changes per 10 red lettered-nucleotides; B) up to 4, 5, or 6 (e.g., up to 1 or 2) nucleotide changes per 10 black lettered-nucleotides; and/or C) up to 7, 8, or 9 (e.g., up to 3 or 4) nucleotide changes per 10 grey lettered-nucleotides. Optionally, the variant of 1) further comprises: a) 7, 8, 9, or 10 (e.g., 9 or 10) nucleotides present per 10 red-circled nucleotides; b) 6, 7, 8, 9, or 10 (e.g., 8, 9 or 10) nucleotides present per 10 black-circled nucleotides; c) 4, 5, 6, 7, 8, 9, or 10 (e.g., 6, 7, 8, 9 or 10) nucleotides present per 10 grey-circled nucleotides; and/or d) 2, 3, 4, 5, 6, 7, 8, 9, or 10 (e.g., 4, 5, 6, 7, 8, 9, or 10) nucleotides present per 10 white-circled nucleotides.

The engineered retron may be engineered by introducing the sequence modifications (e.g., deletions, additions, or substitutions) into the wild-type retron encoding wild-type retron ncRNA, or into the retron encoding the consensus retron ncRNA.

For example, a variant retron may not satisfy the sequence and/or structural requirements of any one of SEQ ID NOs: of Table B and the corresponding FIGS. 2-27, respectively, but may still be a suitable template for the engineered retron described herein, so long as one or more of the conditions set forth in A)-C) and/or a)-d) are met.

In certain embodiments, the highly conserved sequences in the template retrons are preserved/conserved or substantially preserved/conserved in the engineered retron described herein.

In certain embodiments, all or substantially all the red lettered-nucleotides (i.e., those conserved in about 97% or more of the retrons in the same clade) are preserved/conserved in the engineered retron described herein. In certain embodiments, no more than 1, 2, or 3 (e.g., up to 1) nucleotide change(s) (e.g., deleted or substituted) occur per 10 red lettered-nucleotides in the engineered retron described herein. In certain embodiments, no more than about 0.3%, 0.5%, 1%, 2%, 3%, 4%, or 5% of the red lettered-nucleotides are changed (e.g., deleted or substituted) in the engineered retron described herein.

In certain embodiments, all or substantially all the black lettered-nucleotides (i.e., those conserved in about 90-97% of the retrons in the same clade) are preserved/conserved in the engineered retron described herein. In certain embodiments, no more than 1, 2, 3, 4, 5, 6, 7, 8, 9, or 10 (e.g., up to 1 or 2) nucleotide change(s) (e.g., deleted or substituted) occur per 10 black lettered-nucleotides are changed in the engineered retron described herein. In certain embodiments, no more than about 3%, 4%, 5% or 10% of the black lettered-nucleotides are changed (e.g., deleted or substituted) in the engineered retron described herein.

In certain embodiments, all or substantially all the grey lettered-nucleotides (i.e., those conserved in about 75-90% of the retrons in the same clade) are preserved/conserved in the engineered retron described herein. In certain embodiments, no more than 1, 2, 3, 4, 5, 6, 7, 8, 9, 10, 11, 12, 13, 14, or 15 (e.g., up to 3 or 4, or up to 7, 8, or 9) nucleotide change(s) (e.g., deleted or substituted) occur per 10 grey lettered-nucleotides are changed in the engineered retron described herein. In certain embodiments, no more than about 5%, 10%, 15%, 20%, or 25% of the grey lettered-nucleotides are changed (e.g., deleted or substituted) in the engineered retron described herein.

In certain embodiments, all or substantially all the red circled-nucleotides (i.e., those with a nucleotide in about 97% or more of the retrons in the same clade) are present in the engineered retron described herein. In certain embodiments, no more than 1, 2, or 3 (e.g., 0.3, 0.5, or up to 1) nucleotides are absent (e.g., deleted) per 10 red circled-nucleotides in the engineered retron described herein. In certain embodiments, 7, 8, 9, or 10 (e.g., 9 or 10) nucleotides are present per 10 red circled-nucleotides in the engineered retron described herein. In certain embodiments, no more than about 0.3%, 0.5%, 1%, 2%, 3%, 4%, or 5% of the red circled-nucleotides are absent (e.g., deleted) in the engineered retron described herein.

In certain embodiments, all or substantially all the black circled-nucleotides (i.e., those with a nucleotide in about 90-97% of the retrons in the same clade) are present in the engineered retron described herein. In certain embodiments, no more than 1, 2, 3, or 4 (e.g., up to 1 or 2) nucleotides are absent (e.g., deleted) per 10 black circled-nucleotides in the engineered retron described herein. In certain embodiments, 6, 7, 8, 9, or 10 (e.g., 8, 9 or 10) nucleotides are present per 10 black circled-nucleotides in the engineered retron described herein. In certain embodiments, no more than about 1%, 2%, 3%, 5% or 10% of the black circled-nucleotides are absent (e.g., deleted) in the engineered retron described herein.

In certain embodiments, all or substantially all the grey circled-nucleotides (i.e., those with a nucleotide in about 75-90% of the retrons in the same clade) are present in the engineered retron described herein. In certain embodiments, no more than 1, 2, 3, 4, or 5 (e.g., up to 2, 3, or 4) nucleotides are absent (e.g., deleted) per 10 grey circled-nucleotides in the engineered retron described herein. In certain embodiments, 4, 5, 6, 7, 8, 9, or 10 (e.g., 6, 7, 8, 9 or 10) nucleotides are present per 10 grey circled-nucleotides in the engineered retron described herein. In certain embodiments, no more than about 5%, 10%, 15%, 20%, or 25% of the grey circled-nucleotides are absent (e.g., deleted) in the engineered retron described herein.

In certain embodiments, all or substantially all the white circled-nucleotides (i.e., those with a nucleotide in about 50-75% of the retrons in the same clade) are present in the engineered retron described herein. In certain embodiments, no more than 1, 2, 3, 4, 5, 6, or 6 (e.g., up to 2, 3, 4, 5, 6) nucleotide are absent (e.g., deleted) per 10 white circled-nucleotides in the engineered retron described herein. In certain embodiments, 2, 3, 4, 5, 6, 7, 8, 9, or 10 (e.g., 4, 5, 6, 7, 8, 9 or 10) nucleotides are present per 10 grey circled-nucleotides in the engineered retron described herein. In certain embodiments, no more than about 5%, 10%, 15%, 20%, 30%, 40%, or 50% of the white circled-nucleotides are absent (e.g., deleted) in the engineered retron described herein.

In some embodiments, the engineered retron is synthetically produced. In other embodiments, the synthetically produced engineered retron comprises the sequences and/or secondary structures as depicted in any one of SEQ ID NOs: of Table B and the corresponding FIGS. 2-27, respectively, and at least the conserved color lettered nucleotides according to their respective levels of sequence identity (e.g., red, black and gray letters), and/or at least the conserved colored circle nucleotides according to their respective levels of probability of sequence presence (e.g., red, black and gray circles).

In some embodiments, the sequence modification in the engineered retrons leads to/results in the encoded retron ncRNA having the desired functional improvement.

In certain embodiments, the one or more sequence modifications comprises, in the ncRNA, one or more of: (i) a modified (e.g., mutated, reduced, or eliminated) bulge in a1, a2, or both a1 and a2; (ii) an extension or shortening of a1, a2, or both a1 and a2; (iii) an extension or shortening of a spacer sequence between hairpin loops (e.g., S1, S2, S3, and/or S4 in FIG. 2, or any of the S regions in an one of FIGS. 2-27); (iv) an additional or modified (e.g., mutated or eliminated) bulge in hairpin loops (e.g., L2 and/or L3 in FIG. 2, or any of the L regions in an one of FIGS. 2-27 (e.g., by removing unpaired bases in the bulge, or by replacing unpaired bases with an equivalent number of base pairs)); (v) a modified (e.g., extended or shortened) length of hairpin loops (e.g., L1, L2, L3, and/or L4 in FIG. 2, or any of the L regions in an one of FIGS. 2-27); (vi) an alternative L1 and/or L2 (in FIG. 2, or any of the L regions in an one of FIGS. 2-27) having complement, reverse, or reverse complement sequences; (vii) a modified (e.g., increased) number of unpaired bases at the tip of hairpin loops (e.g., L1, L2, L3, and/or L4 in FIG. 2, or any of the L regions in an one of FIGS. 2-27); (viii) a modified (e.g., increased or decreased) GC content in hairpin loops (e.g., L1, L2, L3, and/or L4 in FIG. 2, or any of the L regions in an one of FIGS. 2-27); (ix) an insertion of the heterologous nucleic acid in spacer sequences between hairpin loops (e.g., S1, S2, S3 and/or S4 in FIG. 2, or any of the S regions in an one of FIGS. 2-27), or at the tip of hairpin loops (e.g., L1, L2, L3, and/or L4 in FIG. 2, or any of the L regions in an one of FIGS. 2-27); (x) a deletion of one or more hairpin loops (e.g., L1, L2, L3 and/or L4 in FIG. 2, or any of the L regions in any one of FIGS. 2-27); (xi) an addition of a new loop in a spacer sequence between hairpin loops (e.g., S1, S2, S3, and/or S4 in FIG. 2, or any of the S regions in any one of FIGS. 2-27); (xii) circularization of the ncRNA with the 5′ end and the 3′ end of the ncRNA being connected either directly, or via a spacer sequence; (xiii) a repositioned branching guanosine capable of initiating reverse transcription priming; (xiv) a staggered end sequence that reduces immunogenicity of the retron ncRNA, created by, e.g., adding or removing the 5′ al nucleotides and/or the 3′ a2 nucleotides; and/or, (xv) an antisense sequence complementary to a CRISPR/Cas guide RNA (gRNA) sequence encoded by the heterologous nucleic acid, wherein the antisense sequence hybridizes to and inhibits said gRNA in the encoded retron ncRNA, and wherein said antisense sequence is removed upon reverse transcription of the msDNA.

Unless specifically indicated otherwise, the a1 and a2 regions are both single-stranded and substantially reverse complementary to each other, forming a stem with optional interruption by a symmetric or asymmetric bulge, with optional one or more 5′ and/or 3′ overhang/unpaired nucleotide(s), wherein the al region generally ends before (e.g., ends immediately 5′ to) the conserved branching guanosine (G) providing the 2′-OH for reverse transcription priming.

In some embodiments, the sequence change comprises a mutated, reduced, or eliminated bulge in the a1/a2 stem region, including sequence change(s) in one (i.e., al or a2) strand, or both a1 and a2 strands.

For example, in some embodiments, the sequence change comprises deleting nucleotides from a1, a2, or both a1 and a2, such that the size of the bulge is reduced, or a symmetrical bulge becomes asymmetrical or vice versa, or a bulge is eliminated.

In some embodiments, the sequence change comprises replacing/substituting nucleotides in a1, a2, or both a1 and a2, such that previously unpaired bases in the bulge become base-paired.

In some embodiments, the sequence change comprises replacing an unpaired purine base with one or more unpaired pyrimidine base(s).

In some embodiments, the sequence change comprises replacing an unpaired pyrimidine base with one or more unpaired purine base(s).

In some embodiments, the sequence change comprises replacing one unpaired purine base (e.g., A or G) with another unpaired purine base (e.g., G or A, respectively).

In some embodiments, the sequence change comprises replacing one unpaired pyrimidine base (e.g., T/U or C) with another unpaired pyrimidine base (e.g., C or T/U, respectively).

In some embodiments, the sequence change comprises an extension or shortening of a1, a2, or both a1 and a2.

For example, the length of al can be shortened by deleting 5′ overhang, deleting any upstream bulge nucleotides, deleting bases involved in base-pairing. Likewise, the length of al can be extended by adding 5′ overhang, adding any upstream bulge nucleotides, adding bases involved in base-pairing.

In some embodiments, the length of a2 can be shortened by deleting 5′ overhang, deleting any downstream bulge nucleotides, deleting bases involved in base-pairing. Likewise, the length of a2 can be extended by adding 5′ overhang, adding any downstream bulge nucleotides, adding bases involved in base-pairing.

In some embodiments, the spacer sequences between hairpin loops as depicted in any one of FIGS. 2-27, (e.g., S1, S2, S3 and/or S4 in FIG. 2) can be extended or shortened. In some embodiments the modification can be by inserting a heterologous nucleic acid sequence in spacer sequences between hairpin loops (e.g., S1, S2, S3 and/or S4 in FIG. 2). In certain embodiments, one or more heterologous nucleic acid sequences is inserted in a spacer sequence in the msd region. In some embodiments, the modification of the spacer region can be by interrupting the spacer with additional bulges or hairpin loops.

In other embodiments, a bulge in the hairpin loops are mutated or eliminated (e.g., by removing unpaired bases) in the bulge, such that, for example, a symmetric bulge becomes an unsymmetrical bulge, or an unsymmetrical bulge becomes a symmetric one or an even more unsymmetrical one. In certain embodiments, unpaired bases in the bulge is replaced with an equivalent number of base pairs. The additional base pairs may be merged into the stem at one or both ends of the previous bulge, or may bisect a previous bulge to create two bulges.

In some embodiments, the length of one or more hairpin loops as depicted in any one of FIGS. 2-27, (e.g., L1, L2, L3 and/or L4 of FIG. 2) can be extended or shortened. For example, the number of unpaired bases within the tip or loop can be increased or decreased. Further, a heterologous nucleic acid sequence of interest can be inserted within the tip or the hairpin loop. In certain embodiments, the heterologous nucleic acid sequence of interest is inserted within the tip or the hairpin loop in the msd locus.

In other embodiments, the GC content in the tip or hairpin loops are increased or decreased.

In still other embodiments, a hairpin loop can be deleted.

In some embodiments, the ncRNA with the 5′ end and the 3′ end of the ncRNA can be circularized by being connected either directly, or via a spacer sequence.

In some embodiments, one or more hairpin loops (e.g. L1, L2, L3 and/or L4 of FIG. 2) are modified to have complement, reverse, or reverse complement sequences.

In certain embodiments, the branching guanosine (G) capable of initiating reverse transcription priming is repositioned. For example, the G can be placed further downstream of the end of the al sequence by, for example, 1, 2, 3, 4, or 5 additional nucleotides.

In certain embodiments, immunogenicity of the retron ncRNA is reduced by, e.g., adding or removing the 5′ al nucleotides and/or the 3′ a2 nucleotides.

In certain embodiments, the one or more heterologous nucleic acid sequences (inserted into the subject engineered retron) comprise: a) a heterologous nucleic acid (such as the coding sequence for an RNA aptamer or a ribozyme) inserted into the msr locus or the msd locus (such as in an S region (e.g., S1, S2, S3 and/or S4 in FIG. 2, or any of the S regions in any one of FIGS. 2-27), or the tip of an L region (e.g., L1, L2, L3 and/or L4 in FIG. 2, or any of the L regions in any one of FIGS. 2-27), or upstream or downstream of either the msr locus or the msd locus; or b) a first heterologous nucleic acid inserted into the msd locus, and a second heterologous nucleic acid inserted either upstream of the msr locus or downstream of the msd locus, wherein the second heterologous nucleic acid encodes a CRISPR/Cas guide RNA (gRNA).

In certain embodiments, an antisense sequence complementary to a CRISPR/Cas guide RNA (gRNA) sequence encoded by the heterologous nucleic acid can be included, wherein the antisense sequence hybridizes to and inhibits the gRNA in the encoded retron ncRNA, and wherein the antisense sequence is removed upon reverse transcription of the msDNA.

In certain embodiments, said heterologous nucleic acid encodes a protein or peptide of interest, or wherein said heterologous nucleic acid comprises or encodes a donor/template sequence (e.g., a donor that corrects/repairs/removes a mutation at the target genome site, such as a mutated exon in a disease gene; a functional DNA element (such as a promoter, an enhancer, a protein binding sequence, a methylation site, a homology region for assisting gene editing, etc.); or a coding sequence for a functional RNA element (ncRNAs, etc.)).

In certain embodiments, the protein or peptide of interest comprises a therapeutic protein (such as a wildtype protein defective in a disease cell, or a therapeutic antibody or antigen-binding fragment thereof) useful in treating a disease.

Other heterologous nucleic acids of the invention are described in other section of the specification, all incorporated herein by reference.

In some embodiments, the template/wild-type retron for the engineered retron encodes a wild-type or consensus retron ncRNA polynucleotide having a consensus secondary structure shown in any one of FIGS. 2-27, which are described individually below:

Variants of this template, which can also be used in the engineered retron of the invention, include a variant having: A) up to 1, 2, or 3 (e.g., up to 1) nucleotide changes per 10 red lettered-nucleotides; B) up to 4, 5, or 6 (e.g., up to 1 or 2) nucleotide changes per 10 black lettered-nucleotides; and/or C) up to 7, 8, or 9 (e.g., up to 3 or 4) nucleotide changes per 10 grey lettered-nucleotides; and/or optionally further comprising: a) 7, 8, 9, or 10 (e.g., 9 or 10) nucleotides present per 10 red-circled nucleotides; b) 6, 7, 8, 9, or 10 (e.g., 8, 9 or 10) nucleotides present per 10 black-circled nucleotides; c) 4, 5, 6, 7, 8, 9, or 10 (e.g., 6, 7, 8, 9 or 10) nucleotides present per 10 grey-circled nucleotides; and/or d) 2, 3, 4, 5, 6, 7, 8, 9, or 10 (e.g., 4, 5, 6, 7, 8, 9, or 10) nucleotides present per 10 white-circled nucleotides.

In some embodiments, the engineered retron is entirely synthetically produced and having the conserved nucleotides as denoted by the colored letters as shown in SEQ ID NO. XX and FIGS. 2-27.

In some embodiments, the non-coding RNA (ncRNA) portion of the engineered retron comprises a polynucleotide (e.g., a DNA molecule) encoding an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B. In some embodiments, the ncRNA does not comprise an ncRNA associated with the sequences of Table X.

Amplification of an engineered retron described herein may be performed, for example, before transfection of cells or ligation into vectors. Any method for amplifying the engineered retron may be used, including, but not limited to polymerase chain reaction (PCR), isothermal amplification, nucleic acid sequence-based amplification (NASBA), transcription mediated amplification (TMA), strand displacement amplification (SDA), and ligase chain reaction (LCR). In one embodiment, the engineered retron comprise common 5′ and 3′ priming sites to allow amplification of retron sequences in parallel with a set of universal primers. In another embodiment, a set of selective primers is used to selectively amplify a subset of retron sequences from a pooled mixture.

In some embodiments, the template/wild-type retron for the engineered retron encodes a wild-type or consensus retron ncRNA polynucleotide having a consensus secondary structure shown in FIG. 2-27, and as described individually below:

### Type IA/IIA1 Retron (FIG. 2)

In some embodiments, the template/wt retron for the subject engineered retron encodes a wild-type retron ncRNA polynucleotide having a consensus secondary structure that can be described as:

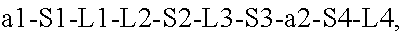

wherein:
a1/a2 is a stem 8 bp in length;
L1 is a stem of 5 bps with a 10-nt tip;
L2 is a stem of 7 bps with a 5-nt tip, and a 1/1 bulge 3 nt from the tip;
L3 is a stem of 23 bps with a 22-nt tip, and a 2/2 bulge 21 bps from the tip;
L4 is a stem of 11 bps with a 5-nt tip;
S1 is a single-stranded spacer region between the a1/a2 stem and L1, with no spacer between L1 and L2;
S2 is a single-stranded spacer region between L2 and L3;
S3 is a single-stranded spacer region between L3 and the a1/a2 stem; and
S4 is a single-stranded spacer region between the a1/a2 stem and L4, and
the conserved nucleotides are as shown in SEQ ID NO: 1 and FIG. 2, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 2.

### Type IB1 Retron (FIG. 3)

In some embodiments, the template/wt retron for the subject engineered retron encodes a wild-type retron ncRNA polynucleotide having a consensus secondary structure that can be described as:

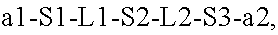

wherein:
a1/a2 is a stem 6 bps in length with a 2/2 bulge 3 bps from the tip, wherein al has a 2-nt overhang and a2 has a 6-nt overhang;
L1 is a stem of 14 bps with a 3-nt tip, a 1/0 bulge 4 bps from the tip, and a 0/6 bulge 10 bps from the tip;
L2 is a stem of 23 bps with a 5-nt tip, a 1/1 bulge 4 bps from the tip, and a 0/1 bulge 18 bps from the tip;
S1 is a single-stranded spacer region between a1/a2 and L1;
S2 is a single-stranded spacer region between L1 and L2;
S3 is a single-stranded spacer region between L2 and a1/a2,
the conserved nucleotides are as shown in FIG. 3, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 3.

### Type IB2 Retron (FIG. 4)

In some embodiments, the template/wt retron for the subject engineered retron encodes a wild-type retron ncRNA polynucleotide having a consensus secondary structure that can be described as:

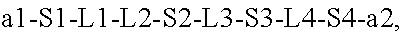

wherein:
the a1/a2 stem is 16 bp in length, with a 17-base 5′ overhang and a 16-base 3′ overhang;
   L1 is a stem of 6 bps with a 4-nt tip;
   L2 is a stem of 4 bps with a 4-nt tip, with a 2/2 bulge 2 nts from the tip;
   L3 is a stem of 3 bps with a 5-nt tip;
   L4 is a stem of 9 bps with a 5-nt tip, and a 1/1 bulge 4 nts from the tip;
   S1, S2, S3, and S4 are single-stranded spacer regions between the a1/a2 stem and L1, L2 and L3, L3 and L4, and L4 and the a1/a2 stem, respectively, with no spacer between L1 and L2; wherein the last 5 nts of S1 and the 5^{th}-9^{th} nts of S2 form a 5-bp stem, and,
the conserved nucleotides are as shown in FIG. 4, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 4.

### Type 1C Retron (FIG. 5)

In some embodiments, the template/wt retron for the subject engineered retron encodes a wild-type retron ncRNA polynucleotide having a consensus secondary structure that can be described as:

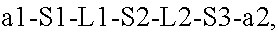

wherein:
a1/a2 is a stem 13 bps in length;
L1 is a stem of 9 bps with a 3-nt tip;
L2 is a stem of 10 bps with a 5-nt tip;
S1 is a single-stranded spacer region between a1/a2 and L1;
S2 is a single-stranded spacer region between L1 and L2;
S3 is a single-stranded spacer region between L2 and a1/a2,
the conserved nucleotides are as shown in FIG. 5, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 5.

### Type IIA1 Retron (FIG. 6)

In some embodiments, the template/wt retron for the subject engineered retron encodes a wild-type retron ncRNA polynucleotide having a consensus secondary structure that can be described as:

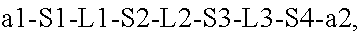

wherein:
a1/a2 is a stem 10 bps in length with a 1-nt overhang on a2;
L1 is a stem of 10 bps with a 3-nt tip;
L2 is a stem of 7 bps with a 5-nt tip;
L3 is a stem of 27 bps with a 8-nt tip and a 0/2 bulge 26 bps from the tip;
S1 is a single-stranded spacer region between a1/a2 and L1;
S2 is a single-stranded spacer region between L1 and L2;
S3 is a single-stranded spacer region between L2 and L3;
S4 is a single-stranded spacer region between L3 and a1/a2,
the conserved nucleotides are as shown in FIG. 6, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 6.

### Type IIA2 Retron (FIG. 7)

In some embodiments, the template/wt retron for the subject engineered retron encodes a wild-type retron ncRNA polynucleotide having a consensus secondary structure that can be described as:

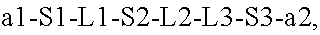

wherein:
a1/a2 is a stem 7 bp in length with no overhangs;
L1 is a stem of 8 bps with a 3-nt tip;
L2 is a stem of 30 bps with a 8-nt tip, a 1/1 bulge 2 bps from the tip, and a 1/1 bulge 27 bps from the tip;
L3 is a stem of 8 bps with a 5-nt tip, and a 0/1 bulge 3 nt from the tip;
S1 is a single-stranded spacer region between the a1/a2 stem and L1;
S2 is a single-stranded spacer region between L1 and L2; and, S3 is a single-stranded spacer region between L3 and the a1/a2 stem;
the conserved nucleotides are as shown in FIG. 7, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 7.

### Type IIA3 Retron (FIG. 8)

In some embodiments, the template/wt retron for the subject engineered retron encodes a wild-type retron ncRNA polynucleotide having a consensus secondary structure that can be described as:

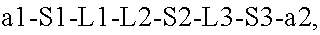

wherein:
a1/a2 is a stem 6 bps in length;
L1 is a stem of 8 bps with a 9-nt tip;
L2 is a stem of 8 bps with a 3-nt tip;
S1 is a single-stranded spacer region between a1/a2 and L1;
S2 is a single-stranded spacer region between L2 and L3;
S3 is a single-stranded spacer region between L3 and a1/a2,
the conserved nucleotides are as shown in FIG. 8, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 8.

### Type IIA4 Retron (FIG. 9)

In some embodiments, the template/wt retron for the subject engineered retron encodes a wild-type retron ncRNA polynucleotide having a consensus secondary structure that can be described as:

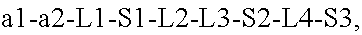

wherein:
a1/a2 is a stem 3 bp in length with no overhangs and a 7-nt tip;
L1 is a stem of 7 bps with a 3-nt tip;
L2 is a stem of 6 bps with a 4-nt tip;
L3 is a stem of 40 bps with a 5-nt tip, and a 2/2 bulge 3 bps from the tip, a 5/4 bulge 10 bps from the tip, and a 12/15 bulge 30 bps from the tip;
L4 is a stem of 4 bps with a 9-nt tip;
S1 is a single-stranded spacer region between L1 and L2/L3;
S2 is a single-stranded spacer region between L2/L3 and L4;
S3 is a single-stranded spacer region between L4 and the 3′ end of the ncRNA; and
the conserved nucleotides are as shown in FIG. 9, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 9.

### Type IIA5 Novel Retron (FIG. 10)

In some embodiments, the template/wt retron for the subject engineered retron encodes a wild-type retron ncRNA polynucleotide having a consensus secondary structure that can be described as:

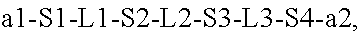

wherein:
a1/a2 is a stem 15 bps in length with a 1-nt overhang on al, a 13-nt overhang on a2, and a 7/5 bulge 13-nt from the tip;
L1 is a stem of 10 bps with a 3-nt tip;
L2 is a stem of 35 bps with a 3-nt tip;
L3 is a stem of 6 bps with a 5-nt tip;
S1 is a single-stranded spacer region between a1/a2 and L1;
S2 is a single-stranded spacer region between L1 and L2;
S3 is a single-stranded spacer region between L2 and L3;
S4 is a single-stranded spacer region between L3 and a1/a2,
the conserved nucleotides are as shown in FIG. 10, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 10.

### Type IIIA1 Retron (FIG. 11)

In some embodiments, the template/wt retron for the subject engineered retron encodes a wild-type retron ncRNA polynucleotide having a consensus secondary structure that can be described as:

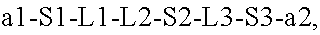

wherein:
a1/a2 is a stem 2 bps in length with a 1-nt overhang on a2;
L1 is a stem of 8 bps with a 4-nt tip;
L2 is a stem of 9 bps with a 3-nt tip and a 1/1 bulge 3 bps from the tip;
L3 is a stem of 20 bps with a 3-nt tip and a 1/2 bulge 3 bps from the tip;
S1 is a single-stranded spacer region between a1/a2 and L1;
S2 is a single-stranded spacer region between L2 and L3;
S3 is a single-stranded spacer region between L3 and a1/a2,
the conserved nucleotides are as shown in FIG. 11, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 11.

### Type IIIA2 Retron (FIG. 12)

In some embodiments, the template/wt retron for the subject engineered retron encodes a wild-type retron ncRNA polynucleotide having a consensus secondary structure that can be described as:

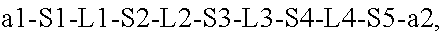

wherein:
a1/a2 is a stem 15 bp in length;
L1 is a stem of 6 bps with a 4-nt tip;
L2 is a stem of 13 bps with a 5-nt tip;
L3 is a stem of 4 bps with a 8-nt tip;
L4 is a stem of 20 bps with a 4-nt tip and a 2/2 bulge 6 bp from the tip;
S1 is a single-stranded spacer region between a1/a2 and L1;
S2 is a single-stranded spacer region between L1 and L2;
S3 is a single-stranded spacer region between L2 and L3;
S4 is a single-stranded spacer region between L3 and L4;
S5 is a single-stranded spacer region between L4 and a1/a2;
the conserved nucleotides are as shown in FIG. 12, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 12.

### Type IIIA3 Retron (FIG. 13)

In some embodiments, the template/wt retron for the subject engineered retron encodes a wild-type retron ncRNA polynucleotide having a consensus secondary structure that can be described as:

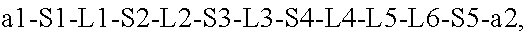

wherein:
a1/a2 is a stem 24 bps in length and having a 1/0 bulge 15 bps from the tip, and a 1/1 bulge 19 bps from the tip;
L1 is a stem of 7 bps with a 4-nt tip;
L2 is a stem of 9 bps with a 8-nt tip;
L3 is a stem of 8 bps with a 4-nt tip;
L4 is a stem of 4 bps with a 9-nt tip, and a 2/2 bulge 3 bps from the tip;
L5 is a stem of 19 bps with a 18-nt tip;
L6 is a stem of 5 bps with a 3-nt tip;
S1 is a single-stranded spacer region between a1/a2 and L1;
S2 is a single-stranded spacer region between L1 and L2;
S3 is a single-stranded spacer region between L2 and L3;
S4 is a single-stranded spacer region between L3 and L4;
S5 is a single-stranded spacer region between L6 and a1/a2;
the conserved nucleotides are as shown in FIG. 13, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 13.

### Type IIIA4 Retron (FIG. 14)

In some embodiments, the template/wt retron for the subject engineered retron encodes a wild-type retron ncRNA polynucleotide having a consensus secondary structure that can be described as:

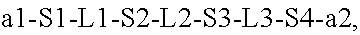

wherein:
a1/a2 is a stem 5 bps in length with a 1/2 bulge 2 bps from the tip;
L1 is a stem of 8 bps with a 6-nt tip;
L2 is a stem of 8 bps with a 5-nt tip;
L3 is a stem of 13 bps with a 14-nt tip and a 1/0 bulge 2 bps from the tip;
S1 is a single-stranded spacer region between a1/a2 and L1;
S2 is a single-stranded spacer region between L1 and L2;
S3 is a single-stranded spacer region between L2 and L3: S4 is a single-stranded spacer region between L3 and a1/a2,
the conserved nucleotides are as shown in SEQ ID NO: 19 and FIG. 20, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 14.

### Type IIIA5 Retron (FIG. 15)

In some embodiments, the template/wt retron for the subject engineered retron encodes a wild-type retron ncRNA polynucleotide having a consensus secondary structure that can be described as:

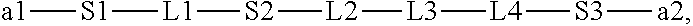

wherein:
the a1/a2 stem is 11 bp in length with no overhang;
L1 is a stem of 9 bps with a 3-nt tip;
L2 is a stem of 14 bps with a 5-nt tip;
L3 is a stem of 9 bps with a 7-nt tip;
L4 is a stem of 15 bps with a 7-nt tip;
S1, S2, and S3 are single-stranded spacer regions between the a1/a2 stem and L1, L2 and L3, and L4 and the a1/a2 stem, respectively, with no spacer between L1 and L2, and no spacer between L2 and L3; wherein the 5^{th}-2^{nd} last nts of S2 and the 3^{rd}-6^{th} nts of S3 forms a 4-bp stem, and,
the conserved nucleotides are as shown in FIG. 15, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 15.

### Type IIIunk Retron (FIG. 16)

In some embodiments, the template/wt retron for the subject engineered retron encodes a wild-type retron ncRNA polynucleotide having a consensus secondary structure that can be described as:

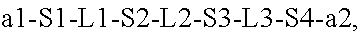

wherein:
a1/a2 is a stem 11 bps in length;
L1 is a stem of 12 bps with a 2-nt tip;
L2 is a stem of 21 bps with a 1-nt tip;
L3 is a stem of 20 bps with a 4-nt tip;
S1 is a single-stranded spacer region between a1/a2 and L1;
S2 is a single-stranded spacer region between L1 and L2;
S3 is a single-stranded spacer region between L2 and L3;
S4 is a single-stranded spacer region between L3 and a1/a2,
the conserved nucleotides are as shown in FIG. 16, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 16.

### Type IV Retron (FIG. 17)

In some embodiments, the template/wt retron for the subject engineered retron encodes a wild-type retron ncRNA polynucleotide having a consensus secondary structure that can be described as:

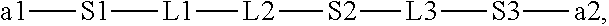

wherein:
a1/a2 is a stem 9 bp in length with no overhang;
L1 is a stem of 5 bps with a 6-nt tip;
L2 is a stem of 9 bps with a 4-nt tip;
L3 is a stem of 26 bps with a 5-nt tip, a 0/1 bulge 7 bps from the tip, and a 0/1 bulge 9 bps from the tip;
S1 is a single-stranded spacer region between the a1/a2 stem and L1, with no spacer region between L1 and L2;
S2 is a single-stranded spacer region between L2 and L3; and, S3 is a single-stranded spacer region between L3 and the a1/a2 stem;
the conserved nucleotides are as shown in FIG. 17, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 17.

### Type IX Retron (FIG. 18)

In some embodiments, the template/wt retron for the subject engineered retron encodes a wild-type retron ncRNA polynucleotide having a consensus secondary structure that can be described as:

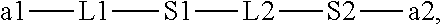

wherein:
a1/a2 is a stem 12 bp in length, wherein al has a 14-nt overhang and a2 has a 2-nt overhang;
L1 is a stem of 11 bps with a 3-nt tip and a 1/3 bulge 7 bp from the tip;
L2 is a stem of 25 bps with a 7-nt tip;
S1 is a single-stranded spacer region between L1 and L2;
S2 is a single-stranded spacer region between L2 and a1/a2;
the conserved nucleotides are as shown in FIG. 18, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 18.

### Type V Retron (FIG. 19)

In some embodiments, the template/wt retron for the subject engineered retron encodes a wild-type retron ncRNA polynucleotide having a consensus secondary structure that can be described as:

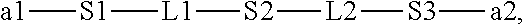

wherein:
a1/a2 is a stem 13 bps in length;
L1 is a stem of 20 bps with a 4-nt tip and a 6/4 bulge 6 bps from the tip;
L2 is a stem of 14 bps with a 4-nt tip and a 1/0 bulge 5 bps from the tip;
S1 is a single-stranded spacer region between a1/a2 and L1;
S2 is a single-stranded spacer region between L1 and L2;
S3 is a single-stranded spacer region between L2 and a1/a2,
the conserved nucleotides are as shown in FIG. 19, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 19.

### Type VI Retron (FIG. 20)

In some embodiments, the template/wt retron for the subject engineered retron encodes a wild-type retron ncRNA polynucleotide having a consensus secondary structure that can be described as:

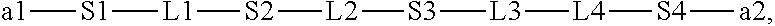

wherein:
a1/a2 is a stem 4 bp in length with a 1 bp 5′ overhang;
L1 is a stem of 7 bps with a 4-nt tip;
L2 is a stem of 8 bps with a 4-nt tip;
L3 is a stem of 16 bps with a 6-nt tip, a 3/4 bulge 3 bps from the tip, a 2/3 bulge 5 bps from the tip, and a 3/1 bulge 8 bps from the tip;
S1 is a single-stranded spacer region between the a1/a2 stem and L1;
S2 is a single-stranded spacer region between L1 and L2;
S3 is a single-stranded spacer region between L2 and L3, with no spacer region between L3 and L4;
S4 is a single-stranded spacer region between L4 and the a1/a2 stem; and,
the conserved nucleotides are as shown in FIG. 20, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 20.

### Type XI Group 1 Retron (FIG. 21)

In some embodiments, the template/wt retron for the subject engineered retron encodes a wild-type retron ncRNA polynucleotide having a consensus secondary structure that can be described as:

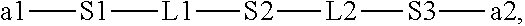

wherein:
a1/a2 is a stem 16 bps in length with a 5-nt overhang on al, and a 3-nt overhang on a2;
L1 is a stem of 9 bps with a 3-nt tip;
L2 is a stem of 7 bps with a 13-nt tip;
S1 is a single-stranded spacer region between a1/a2 and L1;
S2 is a single-stranded spacer region between L1 and L2;
S3 is a single-stranded spacer region between L2 and a1/a2,
the conserved nucleotides are as shown in FIG. 21, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 21.

### Type XI Group 2 Retron (FIG. 22)

In some embodiments, the template/wt retron for the subject engineered retron encodes a wild-type retron ncRNA polynucleotide having a consensus secondary structure that can be described as:

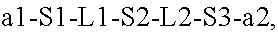

wherein:
a1/a2 is a stem 13 bps in length with a 1-nt overhang on a2;
L1 is a stem of 7 bps with a 3-nt tip and a 2/2 bulge 1 bp from the tip;
L2 is a stem of 8 bps with a 20-nt tip;
S1 is a single-stranded spacer region between a1/a2 and L1;
S2 is a single-stranded spacer region between L1 and L2;
S3 is a single-stranded spacer region between L2 and a1/a2,
the conserved nucleotides are as shown in FIG. 22, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 22.

### Type XII Retron (FIG. 23)

In some embodiments, the template/wt retron for the subject engineered retron encodes a wild-type retron ncRNA polynucleotide having a consensus secondary structure that can be described as:

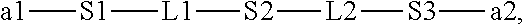

wherein:
a1/a2 is a stem 13 bps in length with a 1-nt overhang on a2;
L1 is a stem of 7 bps with a 3-nt tip, and a 2/2 bulge 1 bp from the tip;
L2 is a stem of 8 bps with a 19-nt tip;
S1 is a single-stranded spacer region between a1/a2 and L1;
S2 is a single-stranded spacer region between L1 and L2;
S3 is a single-stranded spacer region between L2 and a1/a2,
the conserved nucleotides are as shown in FIG. 23, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 23.

### Type XIII Retron (FIG. 24)

In some embodiments, the template/wt retron for the subject engineered retron encodes a wild-type retron ncRNA polynucleotide having a consensus secondary structure that can be described as:

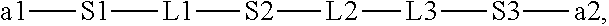

wherein:
a1/a2 is a stem 7 bp in length with no overhangs;
L1 is a stem of 8 bps with a 3-nt tip;
L2 is a stem of 30 bps with a 8-nt tip, a 1/1 bulge 2 bps from the tip, and a 1/1 bulge 27 bps from the tip;
L3 is a stem of 8 bps with a 5-nt tip, and a 0/1 bulge 3 nt from the tip;
S1 is a single-stranded spacer region between the a1/a2 stem and L1;
S2 is a single-stranded spacer region between L1 and L2; and,
S3 is a single-stranded spacer region between L3 and the a1/a2 stem;
the conserved nucleotides are as shown in FIG. 24, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 24.

### Type XIV Retron (FIG. 25)

In some embodiments, the template/wt retron for the subject engineered retron encodes a wild-type retron ncRNA polynucleotide having a consensus secondary structure that can be described as:

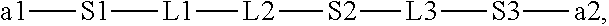

wherein:
the a1/a2 stem is 15 bp in length with no overhang, and a 4/2 bulge 7 bps from the 5′ end of al;
L1 is a stem of 8 bps with a 5-nt tip;
L2 is a stem of 7 bps with a 5-nt tip;
L3 is a stem of 13 bps with a 2-nt tip, a 5/9 bulge 5 bps from the tip, and a 5/5 bulge 8 bps from the tip;
S1, S2, and S3 are single-stranded spacer regions between the a1/a2 stem and L1, L2 and L3, and L3 and the a1/a2 stem, respectively, with no spacer between L1 and L2; and wherein the 5^{th}-3^{rd} last nts of S1 and the 2nd-5th 4 nts of S2 forms a 3-bp stem; and,
the conserved nucleotides are as shown in FIG. 25, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 25.

### Ec107-Like Retron (FIG. 26)

In some embodiments, the template/wt retron for the subject engineered retron encodes a wild-type retron ncRNA polynucleotide having a consensus secondary structure that can be described as:

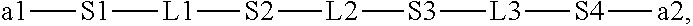

wherein:
a1/a2 is a stem 12 bp in length;
L1 is a stem of 4 bps with a 8-nt tip;
L2 is a stem of 8 bps with a 3-nt tip;
L3 is a stem of 22 bps with a 3-nt tip, a 4/6 bulge 6 bp from the tip, a 3/3 bulge 13 bp from the tip, and a 1/1 bulge 18 bp from the tip;
S1 is a single-stranded spacer region between a1/a2 and L1;
S2 is a single-stranded spacer region between L1 and L2;
S3 is a single-stranded spacer region between L2 and L3; and
S4 is a single-stranded spacer region between L3 and a1/a2,
the conserved nucleotides are as shown in FIG. 26, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 26.

### Outgroup A Retron (FIG. 27)

In some embodiments, the template/wt retron for the subject engineered retron encodes a wild-type retron ncRNA polynucleotide having a consensus secondary structure that can be described as:

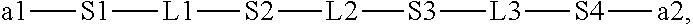

wherein:
a1/a2 is a stem 5 bps in length with a 2-nt overhang on a2;
L1 is a stem of 11 bps with a 4-nt tip;
L2 is a stem of 8 bps with a 3-nt tip;
L3 is a stem of 18 bps with a 3-nt tip;
S1 is a single-stranded spacer region between a1/a2 and L1;
S2 is a single-stranded spacer region between L1 and L2;
S3 is a single-stranded spacer region between L2 and L3;
S4 is a single-stranded spacer region between L3 and a1/a2,
the conserved nucleotides are as shown in FIG. 27, and wherein the colored circled nucleotides are present at the respective levels of certainty (e.g., at least about 97% of the red-circled nucleotides, at least about 90-97% of the black-circled nucleotides, at least about 75-90% of the grey-circled nucleotides, and at least about 50% of the white-circled nucleotides are present).

In some embodiments, the engineered retron is entirely synthetically produced and having the conserved nucleotides as denoted by the colored letters as shown in FIG. 27.

Amplification of an engineered retron described herein (e.g., in FIGS. 2-27) may be performed, for example, before transfection of cells or ligation into vectors. Any method for amplifying the engineered retron may be used, including, but not limited to polymerase chain reaction (PCR), isothermal amplification, nucleic acid sequence-based amplification (NASBA), transcription mediated amplification (TMA), strand displacement amplification (SDA), and ligase chain reaction (LCR). In one embodiment, the engineered retron comprise common 5′ and 3′ priming sites to allow amplification of retron sequences in parallel with a set of universal primers. In another embodiment, a set of selective primers is used to selectively amplify a subset of retron sequences from a pooled mixture.

Variants of these templates, which can also be used in the engineered retron of the invention, include a variant having: A) up to 1, 2, or 3 (e.g., up to 1) nucleotide changes per 10 red lettered-nucleotides; B) up to 4, 5, or 6 (e.g., up to 1 or 2) nucleotide changes per 10 black lettered-nucleotides; and/or C) up to 7, 8, or 9 (e.g., up to 3 or 4) nucleotide changes per 10 grey lettered-nucleotides; and/or optionally further comprising: a) 7, 8, 9, or 10 (e.g., 9 or 10) nucleotides present per 10 red-circled nucleotides; b) 6, 7, 8, 9, or 10 (e.g., 8, 9 or 10) nucleotides present per 10 black-circled nucleotides; c) 4, 5, 6, 7, 8, 9, or 10 (e.g., 6, 7, 8, 9 or 10) nucleotides present per 10 grey-circled nucleotides; and/or d) 2, 3, 4, 5, 6, 7, 8, 9, or 10 (e.g., 4, 5, 6, 7, 8, 9, or 10) nucleotides present per 10 white-circled nucleotides.

In some embodiments, the non-coding RNA (ncRNA) portion of the engineered retron comprises a polynucleotide (e.g., a DNA molecule) encoding an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B. In some embodiments, the ncRNA does not comprise an ncRNA associated with the RT sequences of Table X.

Format of ncRNA and Guide RNA

In certain embodiments, the ncRNA and the guide RNA can be delivered as a single molecule, i.e., with the guide RNA fused to the 5′ and/or 3′ end of the ncRNA. The ncRNA may have guide RNAs located at both ends in some embodiments.

In other embodiments, the guide RNA and ncRNA may be provided and/or delivered as separate components. As shown in Example 4, separation of the guide RNA from the ncRNA can result in increased editing efficiency.

In still other embodiments, a ncRNA-gRNA fusion may be co-delivered with a separate guide RNA.

Modified ncRNAs

In still other embodiments, the ncRNAs disclosed herein may be modified by introducing additional RNA motifs into the ncRNAs, e.g., at the 5′ and 3′ termini of the ncRNAs, or even at positions therein between (e.g., in the msr or msd regions) to improve transcriptional production and/or stability and/or function (e.g., RT-DNA production). Such structures may include, but are not limited to RNA hairpins, RNA step-loops, RNA quadruplexes, cap structures, and poly(A) tails, or ribozyme functions and the like. Also, ncRNAs could also be modified to include one or more nuclear localization sequences.

Additional RNA motifs could also improve RT processivity of the ncRNA or enhance ncRNA activity by enhancing RT binding. Addition of dimerization motifs—such as kissing loops or a GNRA tetraloop/tetraloop receptor pair—at the 5′ and 3′ termini of the ncRNA could also result in effective circularization of the ncRNA, improving stability. Additionally, it is envisioned that addition of these motifs could enable the physical separation of ncRNA components, e.g., separation of the msr and msd regions. Short 5′ extensions or 3′ extensions to the ncRNA that form a small toehold hairpin at either or both ends of the ncRNA could also compete favorably against the annealing of intracomplementary regions along the length of the ncRNA. Finally, kissing loops could also be used to recruit other RNAs or proteins to the genomic site and enable swapping of RT activity from one RNA to the other.

ncRNAs could be further improved via directed evolution, in an analogous fashion to how protein function can be improved. Directed evolution could enhance ncRNA recognition by RT and/or reduce off-site targeting and/or indels and/or improve precise editing efficiency.

The present disclosure contemplates any such ways to further improve the stability and/or functionality of the ncRNAs disclosed here.

In some embodiments, the RNAs (including the guide RNAs and the ncRNAs) used in the compositions of the disclosure have undergone a chemical or biological modification to render them more stable. Exemplary modifications to an RNA include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring RNA, for example, covalent modifications such as the introduction of modified nucleotides, (e.g., nucleotide analogs, or the inclusion of pendant groups which are not naturally found in such mRNA molecules).

Other suitable polynucleotide modifications that may be incorporated into the RNAs used in the compositions of the disclosure include, but are not limited to, 4′-thio-modified bases: 4′-thio-adenosine, 4′-thio-guanosine, 4′-thio-cytidine, 4′-thio-uridine, 4′-thio-5-methyl-cytidine, 4′-thio-pseudouridine, and 4′-thio-2-thiouridine, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyluridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl)adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof. The term modification also includes, for example, the incorporation of non-nucleotide linkages or modified nucleotides into the mRNA sequences of the present invention (e.g., modifications to one or both of the 3′ and 5′ ends of an mRNA molecule encoding a functional protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an RNA molecule (e.g., which form secondary structures).

In some embodiments, RNAs (e.g., ncRNAs) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5′)A and G(5′)ppp(5′)G. Naturally occurring cap structures comprise a 7-methyl guanosine that is linked via a triphosphate bridge to the 5′-end of the first transcribed nucleotide, resulting in a dinucleotide cap of m7G(5′)ppp(5′)N, where N is any nucleoside. In vivo, the cap is added enzymatically. The cap is added in the nucleus and is catalyzed by the enzyme guanylyl transferase. The addition of the cap to the 5′ terminal end of RNA occurs immediately after initiation of transcription. The terminal nucleoside is typically a guanosine, and is in the reverse orientation to all the other nucleotides, i.e., G(5′)ppp(5′)GpNpNp.

Additional cap analogs include, but are not limited to, a chemical structures selected from the group consisting of m7GpppG, m7GpppA, m7GpppC; unmethylated cap analogs (e.g., GpppG); dimethylated cap analog (e.g., m2,7GpppG), trimethylated cap analog (e.g., m2,2,7GpppG), dimethylated symmetrical cap analogs (e.g., m7Gpppm7G), or anti reverse cap analogs (e.g., ARCA; m7,2′OmeGpppG, m72′dGpppG, m7,3′OmeGpppG, m7,3′dGpppG and their tetraphosphate derivatives) (see, e.g., Jemielity, J. et al., “Novel ‘anti-reverse’ cap analogs with superior translational properties”, RNA, 9: 1108-1122 (2003)).

Typically, the presence of a “tail” serves to protect the RNA (e.g., ncRNA) from exonuclease degradation. A poly A or poly U tail is thought to stabilize natural messengers and synthetic sense RNA. Therefore, in certain embodiments a long poly A or poly U tail can be added to an RNA molecule thus rendering the RNA more stable. Poly A or poly U tails can be added using a variety of art-recognized techniques. For example, long poly A tails can be added to synthetic or in vitro transcribed RNA using poly A polymerase (Yokoe, et al. Nature Biotechnology. 1996; 14: 1252-1256). A transcription vector can also encode long poly A tails. In addition, poly A tails can be added by transcription directly from PCR products. Poly A may also be ligated to the 3′ end of a sense RNA with RNA ligase (see, e.g., Molecular Cloning A Laboratory Manual, 2nd Ed., ed. by Sambrook, Fritsch and Maniatis (Cold Spring Harbor Laboratory Press: 1991 edition)).

Typically, the length of a poly A or poly U tail can be at least about 10, 50, 100, 200, 300, 400 at least 500 nucleotides. In some embodiments, a poly-A tail on the 3′ terminus of mRNA typically includes about 10 to 300 adenosine nucleotides (e.g., about 10 to 200 adenosine nucleotides, about 10 to 150 adenosine nucleotides, about 10 to 100 adenosine nucleotides, about 20 to 70 adenosine nucleotides, or about 20 to 60 adenosine nucleotides). In some embodiments, mRNAs include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A or poly U tail or may substitute the poly-A or poly U tail.

RNAs according to the present disclosure (e.g., ncRNAs) may be synthesized according to any of a variety of known methods. For example, RNAs according to the present invention may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application. An improved method of IVT of a ncRNA is disclosed in Example 5 herein.

In a particular embodiment (as exemplified in Example 6 herein), the ncRNAs can comprise an MS2 modification, as specific RNA hairpin structure recognized in nature by a certain MS2-binding protein. This domain can help to stabilize the ncRNA and improve the editing efficiency. The disclosure contemplates other similar modifications. A review of other such MS2-like domains are described in the art, for example, in Johansson et al., “RNA recognition by the MS2 phage coat protein,” Sem Virol., 1997, Vol. 8(3): 176-185; Delebecque et al., “Organization of intracellular reactions with rationally designed RNA assemblies,” Science, 2011, Vol. 333: 470-474; Mali et al., “Cas9 transcriptional activators for target specificity screening and paired nickases for cooperative genome engineering,” Nat. Biotechnol., 2013, Vol. 31: 833-838; and Zalatan et al., “Engineering complex synthetic transcriptional programs with CRISPR RNA scaffolds,” Cell, 2015, Vol. 160: 339-350, each of which are incorporated herein by reference in their entireties. Other systems include the PP7 hairpin, which specifically recruits the PCP protein, and the “com” hairpin, which specifically recruits the Com protein. See Zalatan et al. The nucleotide sequence of the MS2 hairpin (or equivalently referred to as the “MS2 aptamer”) is: GCCAACATGAGGATCACCCATGTCTGCAGGGCC (SEQ ID NO:19935).

### B. Heterologous Nucleotide Sequence (HNS)

The engineered retron may comprise or encode a heterologous nucleic acid (e.g., DNA or RNA) within the msr locus or the msd locus (such as in an S region or the tip of an L region in the consensus structure in any one of FIGS. 2-27, and variants thereof), or upstream or downstream of either the msr locus or the msd locus. In some embodiments, the heterologous nucleic acid is inserted within the msd locus. In some embodiments, the heterologous nucleic acid is inserted upstream of the msr locus. In some embodiments, the heterologous nucleic acid is inserted upstream or downstream of the msd locus. In other embodiments, the heterologous nucleic acid is inserted in spacer sequences (e.g., S1, S2, S3 and/or S4 as depicted in FIG. 2) between hairpin loops and/or within hairpin loops (e.g., L1, L2, L3 and/or L4 as depicted in FIG. 2), e.g., the tip or loop region, or within a bulge. In some embodiments, the heterologous nucleic acid sequence can be inserted in the tip of an L region or hairpin loop (e.g., L1, L2, L3 and/or L4 as depicted in FIG. 2). In some embodiments, one or more heterologous nucleic acids are inserted into the msd locus. In some embodiments, a first heterologous nucleic acid is inserted into the msd locus, and a second heterologous nucleic acid is inserted either upstream of the msr locus or upstream or downstream of the msd locus.

In some embodiments, the heterologous nucleic acid comprises flanking contiguous nucleotides (also referred to as homologous arms) that are substantially complementary to a target site genomic sequence of a cell to facilitate insertion of at least part of the heterologous nucleic acid into the genome of the cell at the target site via homology directed repair (HDR). In some embodiments, the heterologous nucleic acid is between >20 nucleotides and 10,000 nucleotides (e.g., including the flanking homologous arms).

In some embodiments, one or both of the homology arm(s) on the heterologous nucleic acid are 100% identical to a target genomic sequence. In some embodiments, one or both of the homology arm(s) on the heterologous nucleic acid are less than 100% complementary to the target genomic sequence, for example, 99%, 98%, 97%, 96%, 95%, 94%, 93%, 92%, 91%, or 90% identical to a target genomic sequence.

The part of the heterologous nucleic acid to be inserted into the target genome sequence is sometimes referred to as a donor sequence. The donor sequence may be partially identical to the full length or portions thereof of a target genomic sequence, or may be unrelated to the target genomic sequence. The donor sequence may be used, for example, to introduce modifications (e.g., substitutions, deletions, insertions, or a combination thereof) such as mutations or other genetic changes (e.g., genetic elements such as stop codons or a shift in an open reading frame on the target polynucleotide) into its target sequence.

In some embodiments, the heterologous nucleic acid sequence is or encodes a biologically active molecule such as, but not limited to, a therapeutic protein.

Any therapeutic proteins may be encoded by the heterologous nucleic acid.

In some embodiments, the heterologous nucleic acid sequences encodes one or more prophylactically- or therapeutically-active proteins, polypeptides, or other factors.

As a non-limiting example, the heterologous sequences may be or encode an agent that enhances tumor killing activity such as, but not limited to, TRAIL or tumor necrosis factor (TNF), in a cancer. As another non-limiting example, the heterologous sequences may be or encode an agent suitable for the treatment of conditions such as muscular dystrophy (e.g., heterologous sequences are or encode dystrophin or a functional fragment or variant thereof such as the numerous dystrophin minigenes or microdystrophine coding sequences known in the art), cardiovascular disease (e.g., heterologous sequences are or encode SERCA2a, GATA4, Tbx5, Mef2C, Hand2, Myocd, etc.), neurodegenerative disease (e.g., heterologous sequences is or encodes NGF, BDNF, GDNF, NT-3, etc.).

As additional non-limiting example, the heterologous nucleic acid sequence may be or encode an agent that enhances tumor killing activity such as, but not limited to, TRAIL or tumor necrosis factor (TNF), in a cancer. As another non-limiting example, the heterologous nucleic acid sequence may be or encode an agent suitable for the treatment of conditions such as muscular dystrophy (e.g., heterologous nucleic acid sequence is or encodes Dystrophin), cardiovascular disease (e.g., heterologous nucleic acid sequence is or encodes SERCA2a, GATA4, Tbx5, Mef2C, Hand2, Myocd, etc.), neurodegenerative disease (e.g., heterologous nucleic acid sequence is or encodes NGF, BDNF, GDNF, NT-3, etc.), chronic pain (e.g., heterologous nucleic acid sequence is or encodes GlyRal), an enkephalin, or a glutamate decarboxylase (e.g., heterologous nucleic acid sequence is or encodes GAD65, GAD67, or another isoform), lung disease (e.g., heterologous nucleic acid sequence is or encodes CFTR), hemophilia (e.g., heterologous nucleic acid sequence is or encodes Factor VIII or Factor IX), neoplasia (e.g., heterologous nucleic acid sequence is or encodes PTEN, ATM, ATR, EGFR, ERBB2, ERBB3, ERBB4, Notchl, Notch2, Notch3, Notch4, AKT, AKT2, AKT3, HIF, HI Fla, HIF3a, Met, HRG, Bcl2, PPARalpha, PPAR gamma, WT1 (Wilms Tumor), FGF Receptor Family members (5 members: 1, 2, 3, 4, 5), CDKN2a, APC, RB (retinoblastoma), MEN1, VHL, BRCA1, BRCA2, AR (Androgen Receptor), TSG101, IGF, IGF Receptor, Igfl (4 variants), Igf2 (3 variants), Igfl Receptor, Igf2 Receptor, Bax, Bcl2, caspases family (9 members: 1, 2, 3, 4, 6, 7, 8, 9, 12), Kras, Ape), age-related macular degeneration (e.g., heterologous nucleic acid sequence is or encodes Aber, Ccl2, Cc2, cp (ceruloplasmin), Timp3, cathepsin D, Vldlr), schizophrenia (e.g., Neuregulin (Nrgl), Erb4 (receptor for Neuregulin), Complexin-1 (Cplxl), Tphl Tryptophan hydroxylase, Tph2 Tryptophan hydroxylase 2, Neurexin 1, GSK3, GSK3a, GSK3b, 5-HIT (Slc6a4), COMT, DRD (Drdla), SLC6A3, DAOA, DTNBPI, Dao (Daol)), trinucleotide repeat disorders (e.g., HTT (Huntington's Dx), SBMA/SMAXI/AR (Kennedy's Dx), FXN/X25 (Friedrich's Ataxia), ATX3 (Machado-Joseph's Dx), ATXNI and ATXN2 (spinocerebellar ataxias), DMPK (myotonic dystrophy), Atrophin-1 and Atnl(DRPLA Dx), CBP (Creb-BP-global instability), VLDLR (Alzheimer's), Atxn7, Atxn10), fragile X syndrome (e.g., heterologous nucleic acid sequence is or encodes FMR2, FXRI, FXR2, mGLUR5), secretase related disorders (e.g., heterologous nucleic acid sequence is or encodes APH-1 (alpha and beta), Presenilin (Psenl), nicastrin (Ncstn), PEN-2), ALS (e.g., heterologous nucleic acid sequence is or encodes SOD1, ALS2, STEX, FUS, TARD BP, VEGF (VEGF-a, VEGF-b, VEGF-c)), autism (e.g., heterologous nucleic acid sequence is or encodes Mecp2, BZRAP1, MDGA2, Sema5A, Neurexin 1), Alzheimer's disease (e.g., heterologous nucleic acid sequence is or encodes E1, CHIP, UCH, UBB, Tau, LRP, PICALM, Clusterin, PSI, SORL1, CR1, Vldlr, Ubal, Uba3, CHIP28 (Aqpl, Aquaporin 1), Uchll, Uchl3, APP), inflammation (e.g., heterologous nucleic acid sequence is or encodes IL-10, IL-1 (IL-Ia, IL-Ib), IL-13, IL-17 (IL-17a (CTLA8), IL-17b, IL-17c, IL-17d, IL-171), 11-23, Cx3crl, ptpn22, TNFa, NOD2/CARD15 for IBD, IL-6, IL-12 (IL-12a, IL-12b), CTLA4, Cx3cll), Parkinson's Disease (e.g., x-Synuclein, DJ-1, LRRK2, Parkin, PINK1), blood and coagulation disorders, such as, e.g., anemia, bare lymphocyte syndrome, bleeding disorders, hemophagocytic lymphohistiocytosis disorders, hemophilia A, hemophilia B, hemorrhagic disorders, leukocyte deficiencies and disorders, sickle cell anemia, and thalassemia (e.g., heterologous nucleic acid sequence is or encodes CRAN1, CDA1, RPS19, DBA, PKLR, PK1, NT5C3, UMPH1, PSNI, RHAG, RH50A, NRAMP2, SPTB, ALAS2, ANHI, ASB, ABCB7, ABC7, ASAT, TAPBP, TPSN, TAP2, ABCB3, PSF2, RING11, MHC2TA, C2TA, RFX5, RFXAP, RFX5, TBXA2R, P2RX1, P2X1, HF1, CFH, HUS, MCFD2, FANCA, FAC A, FA1, FA, FA A, FAAP95, FAAP90, FLJ34064, FANCB, FANCC, FACC, BRCA2, FANCDI, FANCD2, FANCD, FACD, FAD, FANCE, FACE, FANCF, XRCC9, FANCG, BR1PI, BACH1, FANCJ, PHF9, FANCL, FANCM, KIAA1596, PRF1, HPLH2, UNC13D, MUNC13-4, HPLH3, HLH3, FHL3, F8, FSC, PI, ATT, F5, ITGB2, CD18, LCAMB, LAD, EIF2B1, EIF2BA, EIF2B2, EIF2B3, EIF2B5, LVWM, CACH, CLE, EIF2B4, HBB, HBA2, HBB, HBD, LCRB, HBA1), B-cell non-Hodgkin lymphoma or leukemia (e.g., heterologous nucleic acid sequence is or encodes BCL7A, BCL7, ALI, TCL5, SCL, TAL2, FLT3, NBS1, NBS, ZNFN1AI, 1KI, LYF1, HOXD4, HOX4B, BCR, CML, PHL, ALL, ARNT, KRAS2, RASK2, GMPS, AFIO, ARHGEF12, LARG, KIAA0382, CALM, CLTH, CEBPA, CEBP, CHIC2, BTL, FLT3, KIT, PBT, LPP, NPMI, NUP214, D9S46E, CAN, CAIN, RUNXI, CBFA2, AMLI, WHSCILI, NSD3, FLT3, AF1Q, NPMI, NUMA1, ZNF145, PLZF, PML, MYL, STAT5B, AF1Q, CALM, CLTH, ARL11, ARLTSI, P2RX7, P2X7, BCR, CML, PHL, ALL, GRAF, NF1, VRNF, WSS, NFNS, PTPNII, PTP2C, SHP2, NS1, BCL2, CCND1, PRAD1, BCL1, TCRA, GATA1, GF1, ERYF1, NFE1, ABLI, NQO1, DIA4, NMOR1, NUP214, D9S46E, CAN, CAIN), inflammation and immune related diseases and disorders (e.g., heterologous nucleic acid sequence is or encodes KIR3DL1, NKAT3, NKB1, AMB11, K1R3DS1, IFNG, CXCL12, TNFRSF6, APT1, FAS, CD95, ALPS1A, IL2RG, SCIDX1, SCIDX, IMD4, CCL5, SCYA5, D17S136E, TCP228, IL10, CSIF, CMKBR2, CCR2, CMKBR5, CCCKR5 (CCR5), CD3E, CD3G, AICDA, AID, HIGM2, TNFRSF5, CD40, UNG, DGU, HIGM4, TNFSFS, CD40LG, HIGM1, IGM, FOXP3, IPEX, AIID, XPID, PIDX, TNFRSF14B, TACI), inflammation (e.g., heterologous nucleic acid sequence is or encodes IL-10, IL-1 (IL-IA, IL-IB), IL-13, IL-17 (IL-17a (CTLA8), IL-17b, IL-17c, IL-17d, IL-171), 11-23, Cx3crl, ptpn22, TNFa, NOD2/CARD15 for IBD, IL-6, IL-12 (IL-12a, IL-12b), CTLA4, Cx3cII), JAK3, JAKL, DCLREIC, ARTEMIS, SCIDA, RAG1, RAG2, ADA, PTPRC, CD45, LCA, IL7R, CD3D, T3D, IL2RG, SCIDXI, SCIDX, IMD4), metabolic, liver, kidney and protein diseases and disorders (e.g., heterologous nucleic acid sequence is or encodes TTR, PALB, APOA1, APP, AAA, CVAP, ADI, GSN, FGA, LYZ, TTR, PALB, KRT18, KRT8, CIRHIA, NAIC, TEX292, KIAA1988, CFTR, ABCC7, CF, MRP7, SLC2A2, GLUT2, G6PC, G6PT, G6PT1, GAA, LAMP2, LAMPB, AGL, GDE, GBE1, GYS2, PYGL, PFKM, TCF1, HNF1A, MODY3, SCOD1, SCOI, CTNNB1, PDGFRL, PDGRL, PRLTS, AX1NI, AXIN, CTNNB1, TP53, P53, LFS1, IGF2R, MPRI, MET, CASP8, MCH5, UMOD, HNFJ, FJHN, MCKD2, ADMCKD2, PAH, PKU1, QDPR, DHPR, PTS, FCYT, PKHD1, ARPKD, PKD1, PKD2, PKD4, PKDTS, PRKCSH, G19P1, PCLD, SEC63), muscular/skeletal diseases and disorders (e.g., heterologous nucleic acid sequence is or encodes DMD, BMD, MYF6, LMNA, LMN1, EMD2, FPLD, CMDIA, HGPS, LGMDIB, LMNA, LMNI, EMD2, FPLD, CMDIA, FSHMD IA, FSHD1A, FKRP, MDC IC, LGMD2I, LAMA2, LAMM, LARGE, KIAA0609, MDC1D, FCMD, TTID, MYOT, CAPN3, CANP3, DYSF, LGMD2B, SGCG, LGMD2C, DMDA1, SCG3, SGCA, ADL, DAG2, LGMD2D, DMDA2, SGCB, LGMD2E, SGCD, SGD, LGMD2F, CMD1L, TCAP, LGMD2G, CMD1N, TRIM32, HT2A, LGMD2H, FKRP, MDCIC, LGMD21, TTN, CMD1G, TMD, LGMD2J, POMT1, CAV3, LGMD1C, SEPN1, SELN, RSMD1, PLEC1, PLTN, EBS1, LRP5, BMNDI, LRP7, LR3, OPPG, VBCH2, CLCN7, CLC7, OPTA2, OSTMI, GL, TCIRG1, TIRC7, OC116, OPTB1, VAPB, VAPC, ALS8, SMN1, SMA1, SMA2, SMA3, SMA4, BSCL2, SPG17, GARS, SMADI, CMT2D, HEXB, IGHMBP2, SMUBP2, CATF1, SMARD1), neurological and neuronal diseases and disorders (e.g., heterologous nucleic acid sequence is or encodes SOD1, ALS2, STEX, FUS, TARDBP, VEGF (VEGF-a, VEGF-b, VEGF-c), APP, AAA, CVAP, ADI, APOE, AD2, PSEN2, AD4, STM2, APBB2, FE65L1, NOS3, PLAU, URK, ACE, DCPI, ACEI, MPO, PACIPI, PAXIPIL, PTIP, A2M, BLMH, BMH, PSEN1, AD3, Mecp2, BZRAP1, MDGA2, Sema5A, Neurexin 1, GLOl, MECP2, RTT, PPMX, MRX16, MRX79, NLGN3, NLGN4, KIAA1260, AUTSX2, FMR2, FXR1, FXR2, mGLUR5, HD, IT15, PRNP, PRIP, JPH3, JP3, HDL2, TBP, SCA17, NR4A2, NURR1, NOT, TINUR, SNCAIP, TBP, SCA17, SNCA, NACP, PARK1, PARK4, DJI, PARK7, LRRK2, PARK8, PINKI, PARK6, UCHL1, PARK5, SNCA, NACP, PARK1, PARK4, PRKN, PARK2, PDJ, DBH, NDUFV2, MECP2, RTT, PPMX, MRX16, MRX79, CDKL5, STK9, MECP2, RTT, PPMX, MRX16, MRX79, x-Synuclein, DJ-1, Neuregulin-1 (Nrgl), Erb4 (receptor for Neuregulin), Complexin-1 (Cplxl), Tphl Tryptophan hydroxylase, Tph2, Tryptophan hydroxylase 2, Neurexin 1, GSK3, GSK3a, GSK3b, 5-HTT (Slc6a4), CONT, DRD (Drdla), SLC6A, DAOA, DTNBP1, Dao (Daol), APH-l(alpha and beta), Presenilin (Psenl), Nicastrin, (Ncstn), PEN-2, Nosl, Parpl, Natl, Nat2, HTT, SBMA/SMAX1/AR, FXN/X25, ATX3, TXN, ATXN2, DMPK, Atrophin-1, Atnl, CBP, VLDLR, Atxn7, and AtxnlO), and ocular diseases and disorders (e.g., Aber, Ccl2, Cc2, cp (ceruloplasmin), Timp3, cathepsin-D, Vldlr, Ccr2, CRYAA, CRYA1, CRYBB2, CRYB2, PITX3, BFSP2, CP49, CP47, CRYAA, CRYAI, PAX6, AN2, MGDA, CRYBAI, CRYB1, CRYGC, CRYG3, CCL, LIM2, MP19, CRYGD, CRYG4, BFSP2, CP49, CP47, HSF4, CTM, HSF4, CTM, MIP, AQPO, CRYAB, CRYA2, CTPP2, CRYBB1, CRYGD, CRYG4, CRYBB2, CRYB2, CRYGC, CRYG3, CCL, CRYAA, CRYAI, GJA8, CX50, CAE1, GJA3, CX46, CZP3, CAE3, CCM1, CAM, KRIT1, APOA1, TGFBI, CSD2, CDGG1, CSD, BIGH3, CDG2, TACSTD2, TROP2, MlSI, VSX1, RINX, PPCD, PPD, KTCN, COL8A2, FECD, PPCD2, PIP5K3, CFD, KERA, CNA2, MYOC, TIGR, GLCIA, JO AG, GPOA, OPTN, GLC1E, FIP2, HYPL, NRP, CYP1BI, GLC3A, OPA1, NTG, NPG, CYPIBI, GLC3A, CRB1, RP12, CRX, CORD2, CRD, RPGRIPI, LCA6, CORD9, RPE65, RP20, AIPL1, LCA4, GUCY2D, GUC2D, LCA1, CORD6, RDH12, LCA3, ELOVL4, ADMD, STGD2, STGD3, RDS, RP7, PRPH2, PRPH, AVMD, AOFMD, and VMD2).

In some embodiments, the heterologous nucleic acid sequence is or encodes a factor that can affect the differentiation of a cell. As a non-limiting example, the expression of one or more of Oct4, Klf4, Sox2, c-Myc, L-Myc, dominant-negative p53, Nanog, Glisl, Lin28, TFIID, mir-302/367, or other miRNAs can cause the cell to become an induced pluripotent stem (iPS) cell.

In some embodiments, the heterologous nucleic acid sequence is or encodes a factor for transdifferentiating cells. Non-limiting examples of factors include: one or more of GATA4, Tbx5, Mef2C, Myocd, Hand2, SRF, Mespl, SMARCD3 for cardiomyocytes; Ascii, Nurrl, LmxlA, Bm2, Mytll, NeuroDl, FoxA2 for neural cells; and Hnf4a, Foxal, Foxa2 or Foxa3 for hepatic cells.

In certain embodiments, the heterologous nucleic acid encodes a therapeutic antibody, or an antigen-binding fragment thereof.

In certain embodiments, the heterologous nucleic acid encodes a protein for replacement therapy. The protein may be defective in a disease cell or a disease organism/individual, and the wild-type protein or a functional fragment or variant thereof, when delivered by the heterologous nucleic acid to the disease cell/tissue/organism/individual, at least partially or fully restores the lost function of the protein the disease cell/tissue/organism/individual.

### HNS=Donor DNA Template

In some embodiments, the heterologous nucleic acid sequence is a donor DNA template that can be integrated into a host genome via HDR.

In some embodiments, the heterologous nucleic acid sequence is a donor DNA that can serve as a template or primer for recombineering during replication.

In certain embodiments, the heterologous nucleic acid comprises or encodes a donor/template sequence, wherein the donor/template corrects/repairs/removes a mutation at the target genome site. For example, the mutation may be a mutated exon in a disease gene.

In certain embodiments, the donor/template may encode or comprises a functional DNA element, such as a promoter, an enhancer, a protein binding sequence, a methylation site, or a homology region for assisting gene editing, etc.

By “donor DNA” or “donor DNA template” it is meant a single-stranded DNA to be inserted at a site cleaved by a gene-editing nuclease (e.g., a CRISPR/Cas effector protein; a TALEN; a ZFN) (e.g., after dsDNA cleavage, after nicking a target DNA, after dual nicking a target DNA, and the like). The donor DNA template can contain sufficient homology to a genomic sequence at the target site, e.g., 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology.

Approximately 25, 50, 100, or 200 nucleotides, or more than 200 nucleotides, of sequence homology between a donor DNA template and a genomic sequence (or any integral value between 10 and 200 nucleotides, or more) can support homology-directed repair. Donor DNA template can be of any length, e.g., 50 nucleotides or more, 100 nucleotides or more, 250 nucleotides or more, 500 nucleotides or more, 1000 nucleotides or more, 5000 nucleotides or more, etc. A suitable donor DNA template can be from 50 nucleotides to 100 nucleotides, from 100 nucleotides to 500 nucleotides, from 500 nucleotides to 1000 nucleotides, from 1000 nucleotides to 5000 nucleotides, or from 5000 nucleotides to 10,000 nucleotides, or more than 10,000 nucleotides, in length.

As noted above, the donor DNA template comprises a first homology arm and a second homology arm. The first homology arm is at or near the 5′ end of the donor DNA; and comprises a nucleotide sequence that is at least partially complementary to a first nucleotide sequence in a target nucleic acid. The second homology arm is at or near the 3′ end of the donor DNA; and comprises a nucleotide sequence that is at least partially complementary to a second nucleotide sequence in the target nucleic acid. The first and second homology arms can each independently have a length of from about 10 nucleotides to 400 nucleotides; e.g., from 10 nucleotides (nt) to 15 nt, from 15 nt to 20 nt, from 20 nt to 25 nt, from 25 nt to 30 nt, from 30 nt to 35 nt, from 35 nt to 40 nt, from 40 nt to 45 nt, from 45 nt to 50 nt, from 50 nt to 75 nt, from 75 nt to 100 nt, from 100 nt to 125 nt, from 125 nt to 150 nt, from 150 nt to 175 nt, from 175 nt to 200 nt, from 200 nt to 225 nt, from 225 nt to 250 nt, from 250 nt to 275 nt, from 275 nt to 300 nt, from 325 nt to 350 nt, from 350 nt to 375 nt, or from 375 nt to 400 nt.

In certain embodiments, the donor DNA template is used for editing the target nucleotide sequence. In certain embodiments, the donor DNA template comprises one or more mutations to be introduced into the target polynucleotide. Examples of such mutations include substitutions, deletions, insertions, or a combination thereof. In certain embodiments, the mutation causes a shift in an open reading frame on the target polynucleotide. In certain embodiments, the donor polynucleotide alters a stop codon in the target polynucleotide. In certain embodiments, the donor polynucleotide corrects a premature stop codon. The correction can be achieved by deleting the stop codon, or by introducing one or more sequence changes to alter the stop codon to a codon. In certain embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment includes a fragment less than the entire copy of a gene but otherwise provides sufficient nucleotide sequence to restore the functionality of a wild type gene or non-coding regulatory sequence (e.g., sequences encoding long non-coding RNA).

In certain embodiments, the donor DNA template may be used to replace a single allele of a defective gene or defective fragment thereof. In another embodiment, the donor DNA template is used to replace both alleles of a defective gene or defective gene fragment. A “defective gene” or “defective gene fragment” is a gene or portion of a gene that when expressed, fails to generate a functioning protein or non-coding RNA with functionality of the corresponding wild-type gene.

In certain example embodiments, these defective genes may be associated with one or more disease phenotypes. In certain example embodiments, the defective gene or gene fragment is not replaced but the heterologous nucleic acid is used to insert donor polynucleotides that encode gene or gene fragments that compensate for or override defective gene expression such that cell phenotypes associated with defective gene expression are eliminated or changed to a different or desired cellular phenotype. This can be achieved by including the coding sequence of a therapeutic protein, such as a therapeutic antibody or functional fragment thereof, or a wild-type version of a defective protein associated with one or more disease phenotypes.

In certain embodiments, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain embodiments, the donor DNA template manipulates a splicing site on the target polynucleotide. In certain embodiments, the donor DNA template disrupts a splicing site. The disruption may be achieved by inserting the polynucleotide to a splicing site and/or introducing one or more mutations to the splicing site. In certain embodiments, the donor polynucleotide may restore a splicing site. For example, the polynucleotide may comprise a splicing site sequence.

In certain embodiments, the donor DNA template to be inserted has a size from 10 bp to 50 kb in length, e.g., from 50 bp to ˜40 kb, from 100 bp to ˜30 kb, from 100 bp to ˜10 kb, from 100 bp to 300 bp, from 200 bp to 400 bp, from 300 bp to 500 bp, from 400 bp to 600 bp, from 500 bp to 700 bp, from 600 bp to 800 bp, from 700 bp to 900 bp, from 800 bp to 1000 bp, from 900 bp to 1100 bp, from 1000 bp to 1200 bp, from 1100 bp to 1300 bp, from 1200 bp to 1400 bp, from 1300 bp to 1500 bp, from 1400 bp to 1600 bp, from 1500 bp to 1700 bp, from 1600 bp to 1800 bp, from 1700 bp to 1900 bp, from 1800 bp to 2000 bp nucleotides in length.

In certain embodiments, the homologous arm on one or both ends of the sequence to be inserted is independently about 20 bp, 40 bp, 60 bp, 80 bp, 100 bp, 120 bp, or 150 bp.

The first homology arm and the second homology arm of the donor DNA flank a nucleotide sequence (“a nucleotide sequence of interest” or “an intervening nucleotide sequence”) that is to be introduced into a target nucleic acid. The nucleotide sequence of interest can comprise: i) a nucleotide sequence encoding a polypeptide of interest; ii) a nucleotide sequence encoding an exon of a gene; iii) a promoter sequence; iv) an enhancer sequence; v) a nucleotide sequence encoding a non-coding RNA; or vi) any combination of the foregoing.

The donor DNA can provide for gene correction, gene replacement, gene tagging, transgene insertion, nucleotide deletion, gene disruption, gene mutation, etc. For example, the donor DNA can be used to add, e.g., insert or replace, nucleic acid material to a target DNA (e.g. to “knock in” a nucleic acid that encodes a protein, an siRNA, an miRNA, etc.), to add a tag (e.g., 6× His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, enhancer, etc.), to modify a nucleic acid sequence (e.g., introduce a mutation), and the like. For example, the donor DNA can be used to modify DNA in a site-specific, i.e. “targeted”, way; for example gene knock-out, gene knock-in, gene editing, gene tagging, etc., as used in, for example, gene therapy, e.g. to treat a disease; or as an antiviral, antipathogenic, or anticancer therapeutic, the production of genetically modified organisms in agriculture, the large scale production of proteins by cells for therapeutic, diagnostic, or research purposes, the induction of pluripotent stem cells, biological research, the targeting of genes of pathogens for deletion or replacement, etc.

In some cases, the donor DNA comprises a nucleotide sequence encoding a polypeptide of interest. Polypeptides of interest include, e.g., a) functional versions of a polypeptide that comprises one or more amino acid substitutions, insertions, and/or deletions and that exhibits reduced function, e.g., where the reduced function is associated with or causes a pathological condition; b) fluorescent polypeptides; c) hormones; d) receptors for ligands; e) ion channels; f) neurotransmitters; g) and the like.

In some cases, the donor DNA comprises a nucleotide sequence that encodes a wild-type protein that is lacking in the recipient cell. In some cases, the donor DNA encodes a wild type factor (e.g. Factor VII, Factor VIII, Factor IX and the like) involved in coagulation. In some cases, the donor DNA comprises a nucleotide sequence that encodes a therapeutic antibody. In some cases, the donor DNA comprises a nucleotide sequence that encodes an engineered protein or receptor. In some cases, the engineered receptor is a T cell receptor (TCR), a natural killer (NK) receptor (NKR), or a B cell receptor (BCR). In some cases, the engineered TCR or NKR targets a cancer marker (e.g., a polypeptide that is expressed (e.g., over-expressed) on the surface of a cancer cell). In some cases, the donor DNA comprises a nucleotide sequence that encodes a chimeric antigen receptor (CAR). In some cases, the CAR targets a cancer marker. Donor DNAs encoding CAR, TCR, and/or NCR proteins may be folded into DNA origami structures (DNA nanostructures) and delivered into T cells or NK cells in vitro or in vivo.

Non-limiting examples of polypeptides that can be encoded by a donor DNA include, e.g., IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WHITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), vWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAMI1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric Golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member lib), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIFI A (hypoxia inducible factor 1, alpha subunit (basic helix-loop-helix transcription factor)), CXCR4 (chemokine (C-X-C motif) receptor 4), PROC (protein C (inactivator of coagulation factors Va and Villa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid Al), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomer ase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CAB 1NI (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp 1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member Al), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IE17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, Al polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member IB), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide Al), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gpl30, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member Al), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, IA, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREBI (cAMP responsive element binding protein 1), POMC (proopiomelanocortin), RAC1 (ras-related C3 botulinum toxin substrate 1 (rho family, small GTP binding protein Racl)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIMP2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin LI), PCNA (proliferating cell nuclear antigen), IGF2 (insulin like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COLlA2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALC A (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PEC AMI (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YMEl-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-b-methyltransferase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADHIC (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), TNC (tenascin C), TYMS (thymidylate synthetase), SHC1 (SHC (Src homology 2 domain containing) transforming protein 1), LRP1 (low density lipoprotein receptor-related protein 1), SOCS3 (suppressor of cytokine signaling 3), ADH1B (alcohol dehydrogenase IB (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor HI), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQOl (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity Ia, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), Fll (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCFlE (butyrylcholinesterase), LIPE (lipase, hormone-sensitive), PRDX5 (peroxiredoxin 5), ADORA1 (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CF1GA (chromogranin A (parathyroid secretory protein 1)), IAPP (islet amyloid polypeptide), RFIO (rhodopsin), ENPP1 (ectonucleotide pyrophosphatase/phosphodiesterase 1), PTF1LF1 (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha), F2RL3 (coagulation factor II (thrombin) receptor-like 3), CX3CL1 (chemokine (C-X3-C motif) ligand 1), BDKRB1 (bradykinin receptor Bl), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISFI (cytokine inducible SF12-containing protein), GAST (gastrin), MYOC (myocilin, trabecular mesh work inducible glucocorticoid response), ATP1 A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), F1SF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTF1 (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOF1 (apolipoprotein FI (beta-2-glycoprotein I)), S100A8 (S100 calcium binding protein A8), IL11 (interleukin 11), ALOX15 (arachidonate 15-lipoxygenase), FBLN1 (fibulin 1), NR1F13 (nuclear receptor subfamily 1, group FI, member 3), SCD (stearoyl-CoA desaturase (delta-9-desaturase)), GIP (gastric inhibitory polypeptide), CF1 GB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), F1SD11B2 (hydroxy steroid (11-beta) dehydrogenase 2), CALCRL (calcitonin receptor-like), GALNT2 (UDP-N-acetyl-alpha-D-galactosamine.polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adeno virus E1B 19 kDa interacting protein 3), GCKR (glucokinase (hexokinase 4) regulator), S100A12 (S100 calcium binding protein A 12), PADI4 (peptidyl arginine deaminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), insulin, RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNAlC (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11 A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase I, alpha), OPRL1 (opiate receptor-like 1), IMPA1 (inositol(myo)-l(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member Bl), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), cystic fibrosis transmembrane conductance regulator (CFTR), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F1IR (Fll receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), K¹HK (ketohexokinase (fructokinase)), SAT1 (spermidine/spermine Nl-acetyltransferase 1), GGFI (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2 A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RFIOB (ras homolog gene family, member B), LY96 (lymphocyte antigen 96), FOXOl (forkhead box 01), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPRI (nuclear protein, transcriptional regulator, 1), 15-Sep (15 kDa selenoprotein), CILP2 (cartilage intermediate layer protein 2), TERC (telomerase RNA component), GGT2 (gamma-glutamyltransf erase 2), MT-COl (mitochondrially encoded cytochrome c oxidase I), UOX (urate oxidase, pseudogene), a CRISPR/Cas effector polypeptide, an enzymatically active CRISPR/Cas effector polypeptide (e.g., is capable of cleaving a target nucleic acid) and a CRISPR/Cas effector polypeptide that is not enzymatically active (e.g., does not cleave a target nucleic acid, but retains binding to the target nucleic acid). In some cases, the donor DNA encodes a wild-type version of any of the foregoing polypeptides; i.e., the donor DNA can encode a “normal” version that does not include a mutation(s) that results in reduced function, lack of function, or pathogenesis.

In some cases, the donor DNA comprises a nucleotide sequence encoding a fluorescent polypeptide. Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, m PI urn (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, can be encoded.

In some cases, the donor DNA encodes an RNA, e.g., an siRNA, a microRNA, a short hairpin RNA (shRNA), an anti-sense RNA, a riboswitch, a ribozyme, an aptamer, a ribosomal RNA, a transfer RNA, and the like.

A donor DNA can include, in addition to a nucleotide sequence encoding one or more gene products (e.g., an RNA and/or a polypeptide), one or more transcriptional control elements, e.g., a promoter, an enhancer, and the like. In some cases, the transcriptional control element is inducible. In some cases, the promoter is reversible. In some cases, the transcriptional control element is constitutive. In some cases, the promoter is functional in a eukaryotic cell. In some cases, the promoter is a cell type-specific promoter. In some cases, the promoter is a tissue-specific promoter.

The nucleotide sequence of the donor DNA is typically not identical to the target nucleic acid (e.g., genomic sequence) that it replaces. Rather, the donor DNA may contain at least one or more single base changes, insertions, deletions, inversions or rearrangements with respect to the target nucleic acid (e.g., genomic sequence), so long as sufficient homology is present to support homology-directed repair (e.g., for gene correction, e.g., to convert a disease-causing base pair or a non-disease-causing base pair). In some cases, the donor DNA comprises a non-homologous sequence flanked by two regions of homology, such that homology-directed repair between the target DNA region and the two flanking sequences results in insertion of the non-homologous sequence at the target region. Donor DNA may also comprise a vector backbone containing sequences that are not homologous to the DNA region of interest (the target nucleic acid) and that are not intended for insertion into the DNA region of interest (the target nucleic acid). Generally, the homologous region(s) of a donor sequence will have at least 50% sequence identity to a target nucleic acid (e.g., a genomic sequence) with which recombination is desired. In certain cases, 60%, 70%, 80%, 90%, 95%, 98%, 99%, or 99.9% sequence identity is present. Any value between 1% and 100% sequence identity can be present, depending upon the length of the donor polynucleotide.

The donor DNA may comprise certain nucleotide sequence differences as compared to the target nucleic acid (e.g., genomic sequence), where such difference include, e.g. restriction sites, nucleotide polymorphisms, selectable markers (e.g., drug resistance genes, fluorescent proteins, enzymes etc.), etc., which may be used to assess for successful insertion of the donor DNA at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence. In some cases, the donor DNA will include one or more nucleotide sequences to aid in localization of the donor to the nucleus of the recipient cell or to aid in the integration of the donor DNA into the target nucleic acid. For example, in some case, the donor DNA may comprise one or more nucleotide sequences encoding one or more nuclear localization signals (e.g. PKKKRKV (SEQ ID NO: 19399), VSRKRPRP (SEQ ID NO: 19400), QRKRKQ (SEQ ID NO: 19401), and the like (Frietas et al (2009) Cun-Genomics 10:550-7). In some cases, the donor DNA will include nucleotide sequences to recruit DNA repair enzymes to increase insertion efficiency. Fiuman enzymes involved in homology directed repair include MRN-CtIP, BLM-DNA2, Exol, ERCC1, Rad51, Rad52, Ligase 1, RoIQ, PARP1, Ligase 3, BRCA2, RecQ/BLM-ToroIIIa, RTEL, Roid, and Roih (Verma and Greenburg (2016) Genes Dev. 30 (10): 1138-1154). In some cases, the donor DNA is delivered as reconstituted chromatin (Cruz-Becerra and Kadonaga (2020) eLife 2020; 9:e55780 DOI: 10.7554/eLife.55780).

In some cases, the ends of the donor DNA are protected (e.g., from exonucleolytic degradation) by any convenient method and such methods are known to those of skill in the art. For example, one or more dideoxynucleotide residues can be added to the 3′ terminus of a linear molecule and/or self complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor DNA, additional lengths of sequence may be included outside of the regions of homology that can be degraded without impacting recombination.

### HNS=Functional RNA Element

In certain embodiments, the donor/template comprises a coding sequence for a functional RNA element (ncRNAs, siRNA, shRNA, sgRNA, etc.).

In some embodiments, the heterologous nucleic acid sequences encode a functional non-translated RNA. In some embodiments, the functional non-translated RNA is an RNA aptamer or a ribozyme.

In some embodiments, the heterologous nucleic acid of the engineered retrons further includes a unique barcode to facilitate multiplexing. Barcodes may include one or more nucleotide sequences that are used to identify a nucleic acid or cell with which the barcode is associated. Such barcodes may be inserted for example, into the tip/loop region of the msd-encoded DNA. Barcodes can be 3-1000 or more nucleotides in length, 10-250 nucleotides in length, or 10-30 nucleotides in length, including any length within these ranges, such as 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 200, 300, 400, 500, 600, 700, 800, 900, or 1000 nucleotides in length.

In some embodiments, barcodes are also used to identify the position (viz., positional barcode) of a cell, colony, or sample from which a retron originated, such as the position of a colony in a cellular array, the position of a well in a multi-well plate, the position of a tube in a rack, or the location of a sample in a laboratory. In particular, a barcode may be used to identify the position of a genetically modified cell containing a retron. The use of barcodes allows retrons from different cells to be pooled in a single reaction mixture for sequencing while still being able to trace a particular retron back to the colony from which it originated.

In addition, adapter sequences can be added to engineered retron to facilitate high-throughput amplification or sequencing. For example, a pair of adapter sequences can be added at the 5′ and 3′ ends of a retron construct to allow amplification or sequencing of multiple engineered retron simultaneously by the same set of primers.

### HNS=Guide RNA

In some embodiments, the functional non-translated RNA is a CRISPR/Cas guide RNA (gRNA) specific for a target sequence in the mammalian cell. FIG. 1G depicts various configurations of a recombinant retron ncRNA which is modified by inserting a guide RNA at the 5′ end or the 3′ of the ncRNA. The guide RNA also may be provided in trans as a separate construct. In addition, guide RNAs may be placed at both ends of a recombinant retron ncRNA.

The skilled person will understand that selection of an appropriate guide RNA is informed by which RNA-guided nuclease is utilized.

A guide RNA provides target specificity to the complex (the RNP complex) by including a targeting segment, which includes a guide sequence (also referred to herein as a targeting sequence), which is a nucleotide sequence that is complementary to a sequence of a target nucleic acid. The term “guide RNA”, as used herein, refers to an RNA that comprises: i) an “activator” nucleotide sequence that binds to a CRISPR/Cas effector polypeptide (e.g., a class 2 CRISPR/Cas effector polypeptide such as a type II, type V, or type VI CRISPR/Cas endonuclease) and activates the CRISPR/Cas effector polypeptide; and ii) a “targeter” nucleotide sequence that comprises a nucleotide sequence that hybridizes with a target nucleic acid. The “activator” nucleotide sequence and the “targeter” nucleotide sequence can be on separate RNA molecules (e.g., a “dual-guide RNA”); or can be on the same RNA molecule (a “single-guide RNA”). A guide nucleic acid in some cases includes only ribonucleotides. In some cases, a guide nucleic acid includes both ribonucleotides and deoxyribonucleotides.

In some cases, a CRISPR/Cas guide RNA comprises one or more modifications, e.g., a base modification, a backbone modification, a sugar modification, etc., to provide the nucleic acid with a new or enhanced feature (e.g., improved stability, such as improved in vivo stability). Suitable nucleic acid modifications include, but are not limited to: 2′O-methyl modified nucleotides, 2′ fluoro modified nucleotides, locked nucleic acid (LNA) modified nucleotides, peptide nucleic acid (PNA) modified nucleotides, nucleotides with phosphorothioate linkages, and a 5′ cap (e.g., a 7-methylguanylate cap (m7G)). Suitable modified nucleic acid backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included. A CRISPR-Cas guide RNA can also include one or more substituted sugar moieties. Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted Ci to Cio alkyl or C2 to C10 alkenyl and alkynyl. Particularly suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable polynucleotides comprise a sugar substituent group selected from: Ci to Cio lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxy ethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Examples of various CRISPR/Cas effector proteins and CRISPR/Cas guide RNAs (as well as information regarding requirements related to protospacer adjacent motif (PAM) sequences present in targeted nucleic acids) can be found in the art, for example, see Jinek et al., Science. 2012 Aug. 17; 337(6096):816-21; Chylinski et al., RNA Biol. 2013 May; 10(5):726-37; Ma et al., Biomed Res Int. 2013; 2013:270805; Hou et al., Proc Natl Acad Sci USA. 2013 Sep. 24; 110(39):15644-9; Jinek et al., Elife. 2013; 2:e00471; Pattanayak et al., Nat Biotechnol. 2013 September; 31(9):839-43; Qi et al., Cell. 2013 Feb. 28; 152(5): 1173-83; Wang et al., Cell. 2013 May 9; 153(4):910-8; Auer et al., Genome Res. 2013 Oct. 31; Chen et al., Nucleic Acids Res. 2013 Nov. 1; 41(20):e19; Cheng et al., Cell Res. 2013 October; 23(10):1163-71; Cho et al., Genetics. 2013 November; 195(3):1177-80; DiCarlo et al., Nucleic Acids Res. 2013 April; 41(7):4336-43; Dickinson et al., Nat Methods. 2013 October; 10(10):1028-34; Ebina et al., Sci Rep. 2013; 3:2510; Fujii et al., Nucleic Acids Res. 2013 Nov. 1; 41(20):e187; Hu et al., Cell Res. 2013 November; 23(11):1322-5; Jiang et al., Nucleic Acids Res. 2013 Nov. 1; 41(20):e188; Larson et al., Nat Protoc. 2013 November; 8(ll):2180-96; Mali et al., Nat Methods. 2013 October; 10(10):957-63; Nakayama et al., Genesis. 2013 December; 51(12):835-43; Ran et al., Nat Protoc. 2013 November; 8(ll):2281-308; Ran et al., Cell. 2013 Sep. 12; 154(6):1380-9; Upadhyay et al., G3 (Bethesda). 2013 Dec. 9; 3(12):2233-8; Walsh et al., Proc Natl Acad Sci USA. 2013 Sep. 24; 110(39):15514-5; Xie et al., Mol Plant. 2013 Oct. 9; Yang et al., Cell. 2013 Sep. 12; 154(6):1370-9; Briner et al., Mol Cell. 2014 Oct. 23; 56(2):333-9; and U.S. patents and patent applications: U.S. Pat. Nos. 8,906,616; 8,895,308; 8,889,418; 8,889,356; 8,871,445; 8,865,406; 8,795,965; 8,771,945; 8,697,359; 20140068797; 20140170753; 20140179006; 20140179770; 20140186843; 20140186919; 20140186958; 20140189896; 20140227787; 20140234972; 20140242664; 20140242699; 20140242700; 20140242702; 20140248702; 20140256046; 20140273037; 20140273226; 20140273230; 20140273231; 20140273232; 20140273233; 20140273234; 20140273235; 20140287938; 20140295556; 20140295557; 20140298547; 20140304853; 20140309487; 20140310828; 20140310830; 20140315985; 20140335063; 20140335620; 20140342456; 20140342457; 20140342458; 20140349400; 20140349405; 20140356867; 20140356956; 20140356958; 20140356959; 20140357523; 20140357530; 20140364333; and 20140377868; all of which are hereby incorporated by reference in their entirety.

Examples and guidance related to type V or type VI CRISPR/Cas endonucleases and guide RNAs (as well as information regarding requirements related to protospacer adjacent motif (PAM) sequences present in targeted nucleic acids) can be found in the art, for example, see Zetsche et al., Cell. 2015 Oct. 22; 163(3):759-71; Makarova et al., Nat Rev Microbiol. 2015 November; 13(11):722-36; and Shmakov et al., Mol Cell. 2015 Nov. 5; 60(3):385-97.

### C. Reverse Transcriptases (RTs)

Reverse transcriptases (RTs, also known as RNA-directed DNA polymerases) are enzymes present in all three domains of life, which are DNA polymerase using RNA as a template. Reverse transcriptases of the present disclosure are used to reverse transcribe template msd RNA into single-stranded msDNA.

The reverse transcriptase or a functional domain thereof that may be used in the instant invention includes prokaryotic and eukaryotic RT, provided that the RT functions within the host to generate a donor polynucleotide sequence from the RNA template (e.g., an RNA template from the retron transcript ncRNA).

In certain embodiments, suitable RT sequences (including amino acid sequences and the encoding polynucleotide sequences) are provided in Table A.

In certain embodiments, the nucleotide sequence of a native or wild-type RT is modified, for example, using known codon optimization techniques, so that expression within the desired host is optimized.

In certain embodiments, the RT domain of a reverse transcriptase is used in the present invention, so long as it is compatible with the engineered retron of the invention. The domain may include only the RNA-dependent DNA polymerase activity. In certain embodiments, the RT domain is non-mutagenic, i.e., does not cause mutation in the donor polynucleotide (e.g., during the reverse transcriptase process). In certain embodiments, the RT domain may be non-retron RT in origin, e.g., a viral RT or a human endogenous RTs. In certain embodiments, the RT domain is retron RT or DGRs RT. In certain embodiments, the RT may be less mutagenic than a counterpart wildtype RT. In certain embodiments, the RT is not mutagenic.

In some embodiments, a reverse transcriptase is encoded by a retron ret gene, which may accompany the cognate msr and msd loci and specifically recognize the secondary structure of the cognate ncRNA transcript.

In some embodiments, the RT may be obtained from prokaryotic or eukaryotic cells. Most reverse transcriptases (80%) can be phylogenetically clustered into three major lineages: group IT introns, diversity-generating retroelements (DGRs), and retrons. Other clades of RTs include abortive infection (Abi) RTs, CRISPR-Cas-associated RTs, Group II-like (G2L), the unknown groups (UG), and rvt elements.

In some embodiments, the RT gene is a cognate RT, a retron RT from a species within the same species or clade of the cognate RT, or a retron RT not within the same clade of the cognate RT such as an unrelated RT or an engineered RT. In some embodiments, the non-retron related RT are RTs from group II introns, diversity-generating retroelements (DGRs), abortive infection (Abi) RTs, CRISPR-Cas-associated RTs, Group II-like (G2L), the unknown groups (UG), and rvt elements. See Mestre et al., Nucleic Acids Research, Volume 48, Issue 22, 16 Dec. 2020, Pages 12632-12647; and Mestere et al., UG/Abi: “A Highly Diverse Family of Prokaryotic Reverse Transcriptases Associated With Defense Functions,” doi.org/10.1101/2021.12.02.470933 (incorporated herein by reference).

In some embodiments, the RT are from clades related to retron/retron-like sequences. In some embodiments, the RT are selected from RTs provided in Table A. In some embodiments, the RT is not an RT associated with the sequences identified in Table X.

In prokaryotic retron systems, the RT gene is typically located downstream from the ncRNA (msr and msd) locus. In the engineered retron, the RT position can differ from the natural or wild type retrons. In some embodiments, the RT gene can be provided in cis, such as either upstream or downstream of the msr locus or the msd locus. In certain embodiments, the RT gene is provided in trans, such as provided separately in a vector of the vector system described herein, wherein the ncRNA coding msr and msd sequences are provided in a different vector of the vector system described herein.

In some embodiments, the RT is modified (e.g., insertion, deletion, and/or substitution of one or more nucleotide(s)) or codon optimized to enhance activity or processivity.

In certain embodiments, a cryptic stop signal is removed from the RT thereby allowing generation of longer ssDNAs.

In certain embodiments, the RT is from a retron which encodes msDNA as described in U.S. Pat. Nos. 6,017,737; 5,849,563; 5,780,269; 5,436,141; 5,405,775; 5,320,958; CA2,075,515; all of which are incorporated by reference herein in their entireties.

In some embodiments, the engineered retron further comprises a polynucleotide (e.g., a DNA molecule) encoding a reverse transcriptase (RT) or a portion thereof. In some embodiments, the encoded RT or portion thereof is capable of synthesizing a DNA copy of at least a portion of the msd locus encoding the msDNA.

In some embodiments, the polynucleotide (e.g., a DNA molecule) encoding the RT comprises a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide listed in Table A.

In some embodiments, the polynucleotide encoding the RT encodes a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or a polypeptide of Table C.

In some embodiments, the polynucleotide encoding the RT does not comprise a polynucleotide listed in Table X.

Once translated, the RT binds the ncRNA template downstream from the msd locus, forming an RT-RNA complex, and initiating reverse transcription of the RNA towards its 5′ end. Accordingly, in certain aspects the disclosure relates to an engineered nucleic acid-enzyme construct comprising: a. a non-coding RNA (ncRNA) comprising: i) an msr locus encoding the msr RNA portion of a multi-copy single-stranded DNA (msDNA); and ii) an msd locus encoding the msd RNA portion of the msDNA; b. a heterologous nucleic acid inserted at or within a location selected from: the msd locus, upstream of the msr locus, upstream of the msd locus, and downstream of the msd locus; and c. a reverse transcriptase (RT), or a domain thereof comprising: i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or ii) a polypeptide listed in Table C. In some embodiments, the RT does not comprise a polypeptide listed in Table X.

In certain aspects, the disclosure relates to an engineered nucleic acid-enzyme construct comprising: a) a non-coding RNA (ncRNA) comprising: i) an msr locus encoding the msr RNA portion of a multi-copy single-stranded DNA (msDNA); and ii) an msd locus encoding the msd RNA portion of the msDNA, b) a heterologous nucleic acid inserted at or within a location selected from: the msd locus; upstream of the msr locus; upstream of the msd locus; and downstream of the msd locus; and c) a reverse transcriptase (RT), or a portion thereof, wherein the RT is capable of synthesizing a DNA copy of at least a portion of the msd locus encoding the msDNA, and wherein the ncRNA and/or the RT is any one of the invention described herein.

In certain aspects, the disclosure relates to an engineered nucleic acid-enzyme construct comprising: a) a non-coding RNA (ncRNA) comprising: i) an msr locus encoding the msr RNA portion of a multi-copy single-stranded DNA (msDNA); and ii) an msd locus encoding the msd RNA portion of the msDNA; b) a heterologous nucleic acid inserted at or within a location selected from: the msd locus, upstream of the msr locus, upstream of the msd locus, and downstream of the msd locus; and c) a reverse transcriptase (RT) or a domain thereof: wherein the RT comprises: i) an RT listed in Table A, or an RT having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an RT listed in Table A; and/or ii) a consensus sequence listed in Table C; and wherein, the RT does not comprise a sequence from Table X.

In some embodiments of the nucleic-acid enzyme constructs described herein, the ncRNA comprises: i) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B; and/or optionally wherein the ncRNA is not an ncRNA from the retons of Table X.

In some embodiments, the RT is linked to components such as RNA-guided and non-RNA guided nucleases. The linked maybe via s peptide bond or a short linker peptide in a fusion protein. Suitable linker peptides include flexible linkers such as those comprising G or S repeats, such as G₄S repeat units or GS repeat units, with 1-20 repeats, e.g., 1, 2, 3, 4, 5, 6, 7, or 8 repeats.

In certain embodiments, the RT is chemically linked or conjugated to the RNA-guided and non-RNA guided nucleases via non-peptide bonds. Such protein conjugates may be delivered directly to a host cell, either together with the nucleic acid component of the engineered retron described herein, or separately.

In some embodiments, the RT is linked to a DNA-repair modulating biomolecule (e.g., NHEJ peptide inhibitors.

### D. Programmable Nucleases (RNA-Guided Nucleases)

In certain embodiments, the engineered retron (e.g., an engineered nucleic acid construct or engineered nucleic acid-enzyme construct as described herein) may comprise or encode, as one heterologous nucleic acid, a guide RNA (gRNA) suitable for guiding a nuclease to target a particular genomic sequence to be modified. The gRNA includes sequences complementary to a genomic sequence, and therefore can mediate binding of the nuclease-gRNA complex to the genomic target site by hybridization between the guide sequence and the target site sequence.

In certain embodiments, the gRNA can be linked to the ncRNA and/or the msDNA encoded by the engineered retron described herein at the 5′ end of the ncRNA and/or the msDNA. In certain embodiments, the gRNA can be linked to the ncRNA and/or the msDNA encoded by the engineered retron described herein at the 3′ end of the RNA of the ncRNA and/or the msDNA after reverse transcription.

In some embodiments, the nuclease that can form a complex with the gRNA can be any one of the art-recognized clustered regularly interspersed short palindromic repeats (CRISPR) system Cas effector enzymes, which are useful for, e.g., genome editing, including genome editing in mammalian cells or human cells.

For example, a gRNA that can be loaded into a Cas9 or variant thereof may be encoded by the engineered retron, such that the gRNA is transcribed as part of the msDNA. In some embodiments, the gRNA may be linked to the 5′ end of the al region of the msr-encoded sequence in the retron ncRNA, as well as the msDNA after reverse transcription. In some embodiments, the gRNA may be part of the modified msr-region present in the ncRNA and the msDNA produced after reverse transcription (viz., the encoded gRNA is not degraded by the synthesis of the msDNA through reverse transcription of the ncRNA).

Any art recognized CRISPR/Cas effector enzymes or variants thereof (“Cas enzymes”) known to be useful for CRISPR-based genome editing can be used with the engineered retron, though such Cas enzymes may not necessarily be part of the engineered retron, and can (but is not required to) be provided separately. For example, the Cas enzymes can be provided as part of the vector system described herein. The coding sequence of the suitable Cas enzyme can be present on the same vector that provides the engineered retron, or on different vectors. When the engineered retron and the Cas enzymes are present on the same vector, they may be under the transcriptional control of the same promoter, enhancer, or other transcriptional regulatory elements, or may be separately regulated by different promoters, enhancers, and/or other transcriptional regulatory sequences in the vector system.

In some embodiments, the Cas enzyme is a Class 2, Type II CRISPR/Cas effector enzyme, such as Cas9. In some embodiments, the Cas9 is from Streptococcus pyogenes (SpCas9), and the gRNA encoded by the engineered retron comprises both the crisprRNA (crRNA) and the tracrRNA linked together as the single guide RNA (gRNA).

In some embodiments, the Cas9 is from Staphylococcus aureus Cas9 (SaCas9), or an engineered variant thereof such as SaCas9-HF (a high-fidelity variant with genome-wide activity), KKHSaCas9 (which recognizes a 5′-NNGRRT-3′ PAM, and has a 2-4× broader range of target sites in the human genome than the wildtype SaCas9), and microABE1744 (an engineered SaCas9 variant adapted for use in adenine base editing (ABE), with significantly improved on-target editing compared to other nucleases, with a reduced RNA off-target footprint).

In some embodiments, the Cas9 is from Streptococcus thermophilus (StCas9), Neisseria meningitidis (NmCas9), Francisella novicida (FnCas9), or Campylobacter jejuni (CjCas9).

In some embodiments, the Cas9 is from Streptococcus canis (ScCas9), with a less stringent PAM sequence requirement of 5′-NNG-3′ (instead of the more stringent 5′-NGG-3′ for SpCas9).

In some embodiments, the Cas9 is from Staphylococcus auricularis (SauriCas9) (which recognizes a 5′-NNGG-3′ PAM sequence, has high editing activity).

In some embodiments, the Cas enzyme is a Cas9 variant with mutated catalytic domain that retains cleavage specificity, but only nicks a single DNA strand at a desired target sequence instead of creating a double-strand break (DSB). Two such Cas9 nickase variants targeting different strands of the same target sequence may be used together to increase fidelity of creating the DSB, each using a different gRNA that can be provided by the engineered retron.

In some embodiments, the Cas enzyme is a high fidelity Cas9 variant with weakened DNA phosphate backbone interaction (such as SpCas9-IF1) that displays genome-wide specificity and undetectable off-target effects.

In some embodiments, the Cas enzyme is a Cas9 variant known as eSpCas9, which has weakened interactions between the eSpCas9 and its gRNAs with non-exact complementarity with the target DNA sequence, thus providing improved specificity and lower off-target editing rates.

In some embodiments, the Cas enzyme is a hyper-accurate Cas9 variant (HypaCas9), which improves proofreading before cleavage, and thus drastically reduces off-target cleavage.

In some embodiments, the Cas enzyme is a Cas9 variant (FokI-Fused dCas9), which combines the DNA recognition ability by dCas9 with the specificity of an active nuclease FokI. The resulting nuclease cuts the target sequence only after dimerization, which is more difficult to occur at off-target sites, thus resulting in enhanced specificity.

In some embodiments, the Cas enzyme is a Cas9 variant xCas9, which recognizes a broad range of PAM sequences, thus increasing the target sites to 1 in 4 in the genome. In addition, the xCas9 variant also exhibits lower off-target rates than the commonly used SpCas9.

In some embodiments, the Cas enzyme is a Cas9 variant with altered PAM sequence specificities, including the SpG variant with an expanded target range of PAM sequences, and the SpRY variant that can target almost all PAM sequences.

In some embodiments, the Cas enzyme is dCas9 having inactivated catalytic nuclease domains while maintaining the recognition domains that allow guide RNA-mediated targeting to specific DNA sequences. The dCas9 may be further linked to a functional domain with a distinct biological function, such as transcriptional activation/depression, DNA methylation, demethylation, endonuclease (such as FokI), or fluorescent dye. Representative (non-limiting) dCas9-linked functional domains include including dCas9-SAM, dCas9-SunTag, dCas9-VPR, and dCas9-KREB.

In some embodiments, the dCas9 is fused with a catalytic enzyme with cytidine deaminase activity, which converts GC basepair to AT basepair.

In some embodiments, the dCas9 is fused with an engineered RNA adenosine deaminase, which converts AT basepair to GC basepair.

In some embodiments, the Cas enzyme is a Class 2, Type V CRISPR/Cas effector enzyme, such as Cas12a (Cpf1) (Type V-A), Cas12b (C2c1) (Type V-B), Cas12c (C2c3) (Type V-C), Cas12d (CasY) (Type V-D), Cas12e (CasX) (Type V-E), Cas12f (Cas14, C2c10) (Type V-F), Cas12g (Type V-G), Cas12h (Type V-H), Cas12i (Type V-I), Cas12k (C2c5) (Type V-K), or C2c4/C2c8/C2c9 (Type V-U).

In some embodiments, the Cas enzyme is Cas12a or Cpf1 (CRISPR from Prevotella and Francisella 1). Unlike Cas9, Cas12a is well-suited for targeting AT-rich DNA sequences because of its AT-rich PAM sequences. In some embodiments, the Cas12a is FnCas12a (that recognizes PAM sequence 5′-TTN-3′), or AsCas12a or LbCas12a (that recognize 5′-TTTV-3′ PAM sequence), where V is A, G, or C nucleotide. Further, Cas12a creates staggered double-stranded breaks in the target DNA, rather than the blunt-ends generated by SpCas9, thus rendering it more useful for HDR repair. In this embodiment, the subject engineered retron encodes a gRNA suitable for use for Cas12a, in that the gRNA does not require a tracer RNA, and only requires the crRNA.

In some embodiments, the Cas enzyme is a Cas12a variant from Acidaminococcus sp. (enAsCas12a), which has an expanded target range of PAM sequences and significantly higher editing activity compared to wild type Cas12a.

In some embodiments, the Cas enzyme is a high-fidelity Cas12a variant (enAsCas12a-HF1) that reduces off-target editing.

In some embodiments, the Cas enzyme is Cas12b or C201. In some embodiments, the Cas12b is form Alicyclobacillus acidoterrestris (AacCas12b), or from Alicyclobacillus acidiphilus Cas12b (AapCas12b).

In some embodiments, the Cas enzyme is a Cas12b variant that functions at 37° C., such as one form Bacillus hisashii (BhCas12b).

In some embodiments, the Cas enzyme is a BhCas12b variant with higher specificity than SpCas9.

In some embodiments, the Cas enzyme is CasX or Cas12d.

In some embodiments, the Cas enzyme is CasY or Cas12e, and the subject engineered retron encodes a short-complementarity untranslated RNA (scoutRNA) together with crRNA (rather than tracrRNA used in other CRISPR-Cas systems).

In some embodiments, the Cas enzyme is Cas14, from archaea bacteria. Cas14 targets single-stranded (ss) DNA target sequences, does not require PAM sequence for activation, and has collateral activity (i.e., cuts other non-target ssDNA strands non-specifically upon binding the target sequence). Unlike Cas12a, Cas14a requires high fidelity complementarity to the target ssDNA, and is very sensitive to internal seed region mismatches of the ssDNA target substrate.

In some embodiments, the gRNA nuclease is an engineered RNA-guided Fokl nuclease. RNA-guided Fokl nucleases comprise fusions of inactive Cas9 (dCas9) and the Fokl endonuclease (FokI-dCas9), wherein the dCas9 portion confers guide RNA-dependent targeting on Fokl. For a description of engineered RNA-guided Fold nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

In other embodiments, the RNA-guided nuclease can be a non-CRISPER/Cas related nuclease such as transposon-encoded nucleases, IscBs, IscR, or TnpBs.

In some embodiments, the Cas enzyme is a Cas9

In some embodiments, the retron-based editing systems described herein can include any Cas9 equivalent. A Cas9 equivalent is a Cas9-like protein that provides the same or substantially the same function as Cas9. For instance, if Cas9 refers to a type II enzyme of the CRISPR-Cas system, a Cas9 equivalent can refer to a type V or type VI enzyme of the CRISPR-Cas system.

For example, Cas12e (CasX) is a Cas9 equivalent that reportedly has the same function as Cas9 but which evolved through convergent evolution. Thus, the Cas12e (CasX) protein is contemplated to be used with the retron-based editor systems described herein. In addition, any variant or modification of Cas12e (CasX) is conceivable and within the scope of the present disclosure.

In some embodiments, Cas9 equivalents may refer to Cas12e (CasX) or Cas12d (CasY), which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-Cas12e and CRISPR-Cas12d, which are among the most compact systems yet discovered. In some embodiments, Cas9 refers to Cas12e, or a variant of Cas12e. In some embodiments, Cas9 refers to a Cas12d, or a variant of Cas12d. It should be appreciated that other RNA-guided DNA binding proteins may be used as a nucleic acid programmable DNA binding protein (napDNAbp), and are within the scope of this disclosure.

In some embodiments, the Cas9 equivalent comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas12e (CasX) or Cas12d (CasY) protein. In some embodiments, the napDNAbp is a naturally-occurring Cas12e (CasX) or Cas12d (CasY) protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a wild-type Cas moiety or any Cas moiety provided herein.

In various embodiments, the nucleic acid programmable DNA binding proteins include, without limitation, Cas9 (e.g., dCas9 and nCas9), Cas12e (CasX), Cas12d (CasY), Cas12a (Cpf1), Cas12b1 (C2c1), Cas13a (C2c2), Cas12c (C2c3), Argonaute, and Cas12b1. One example of a nucleic acid programmable DNA-binding protein that has different PAM specificity than Cas9 is Clustered Regularly Interspaced Short Palindromic Repeats from Prevotella and Francisella 1 (i.e, Cas12a (Cpf1)). Similar to Cas9, Cas12a (Cpf1) is also a Class 2 CRISPR effector, but it is a member of type V subgroup of enzymes, rather than the type II subgroup. It has been shown that Cas12a (Cpf1) mediates robust DNA interference with features distinct from Cas9. Cas12a (Cpf1) is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

In still other embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Cst2, Csf3, Csf4, homologs thereof, or modified versions thereof.

In various other embodiments, the RNA-guided nuclease can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, or an Argonaute (Ago) domain, or a variant thereof.

Amino acid sequences of RNA-guided nucleases are readily available and known in the art. Exemplary RNA-guided nucleases and their amino acid sequences can be found, for example, in WO 2017/070633, US 2020/0010835, US 2022/0204975, U.S. Pat. No. 11,071,790, WO 2020/191233, U.S. Pat. Nos. 11,447,770, 10,858,639, and 10,947,530, each of which are incorporated herein by reference in their entireties.

### E. Programmable Nucleases (Other)

In addition to the CRISPR/Cas system, the subject engineered retron can also be used in combination with sequence-specific nucleases that do not use a guide RNA to recognize a target sequence, such as non-CRISPR/Cas sequence-specific nucleases including TALENs, ZFNs, meganucleases, and restriction enzymes, as well as other sequence-specific nucleases that use other RNA guides, such as transposon-encoded IscBs, IscR, or TnpBs.

For example, the subject engineered retron may encode or provide a msDNA that can serve as a donor or template sequence for HDR-mediated genome editing. Optionally, the RT of the engineered retron is fused to such sequence-specific nuclease, such that the msDNA, by way of being generated by the RT close to the site of HDR-mediated genome editing, can be more efficiently participate in the HDR-mediated genome editing.

In some embodiments, the non-CRISPR/Cas sequence-specific nuclease is or comprises a TALE Nuclease, a TALE nickase, Zinc Finger (ZF) Nuclease, ZF Nickase, meganuclease, or a combination thereof. In some embodiments, the non-CRISPR/Cas sequence-specific nuclease is or includes two, three, four, or more of an independently selected TALE Nuclease, TALE nickase, Zinc Finger (ZF) Nuclease, ZF Nickase, Meganuclease, restriction enzymes or a combination thereof. In some embodiments, the combination is or comprises a TALE Nuclease/a ZF Nuclease; a TALE Nickase/a ZF nickase.

In some embodiments, the non-CRISPR/Cas sequence-specific nuclease is or comprises a TALE Nuclease (Transcription Activator-Like Effector Nucleases (TALEN)). TALENs are restriction enzymes engineered to cut specific target DNA sequences. TALENs comprise a TAL effector (TALE) DNA-binding domain (which binds at or close to the target DNA), fused to a DNA cleavage domain which cuts target DNA. TALEs are engineered to bind to practically any desired DNA sequence. Thus in some embodiments, the TALEN comprises an N-terminal capping region, a DNA binding domain which may comprise at least one or more TALE monomers or half-monomers specifically ordered to target the genomic locus of interest, and a C-terminal capping region, wherein these three parts are arranged in a predetermined N-terminus to C-terminus orientation. Optionally, the TALEN includes at least one or more regulatory or functional protein domains.

In some embodiments, the TALE monomers or half monomers may be variant TALE monomers derived from natural or wild type TALE monomers but with altered amino acids at positions usually highly conserved in nature, and in particular have a combination of amino acids as RVDs that do not occur in nature, and which may recognize a nucleotide with a higher activity, specificity, and/or affinity than a naturally occurring RVD. The variants may include deletions, insertions and substitutions at the amino acid level, and transversions, transitions and inversions at the nucleic acid level at one or more locations. The variants may also include truncations.

In some embodiments, the TALE monomer/half monomer variants include homologous and functional derivatives of the parent molecules. In some embodiments, the variants are encoded by polynucleotides capable of hybridizing under high stringency conditions to the parent molecule-encoding wild-type nucleotide sequences.

In some embodiments, the DNA binding domain of the TALE has at least 5 of more TALE monomers and at least one or more half-monomers specifically ordered or arranged to target a genomic locus of interest. The construction and generation of TALEs or polypeptides of the invention may involve any of the methods known in the art.

Naturally occurring TALEs or “wild type TALEs” are nucleic acid binding proteins secreted by numerous species of proteobacteria. TALEs contain a nucleic acid binding domain composed of tandem repeats of highly conserved monomer polypeptides that are predominantly 33, 34 or 35 amino acids in length and that differ from each other mainly in amino acid positions 12 and 13. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such monomers, the RVD consists of a single amino acid. In such cases the RVD may be alternatively represented as X*, where X represents X12 and (*) indicates that X13 is absent. The DNA binding domain may comprise several repeats of TALE monomers and this may be represented as (X1-11-(X12X13)-X14-33 or 34 or 35)z, where z is optionally at least 5-40, such as 10-26.

The TALE monomers have a nucleotide binding affinity that is determined by the identity of the amino acids in its RVD. Polypeptide monomers with an RVD of NI preferentially bind to adenine (A), monomers with an RVD of NG preferentially bind to thymine (T), monomers with an RVD of HD preferentially bind to cytosine (C), monomers with an RVD of NN preferentially bind to both adenine (A) and guanine (G), monomers with an RVD of IG preferentially bind to T, monomers with an RVD of NS recognize all four base pairs and may bind to A, T, G or C. Thus, the number and order of the polypeptide monomer repeats in the nucleic acid binding domain of a TALE determines its nucleic acid target specificity. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In some embodiments, the TALE is a dTALE (or designerTALE), see Zhang et al., Nature Biotechnology 29:149-153 (2011), incorporated herein by reference.

In some embodiments, the TALE monomer comprises an RVD of HN or NH that preferentially binds to guanine, and the TALEs have high binding specificity for guanine containing target nucleic acid sequences. In come embodiments, polypeptide monomers having RVDs RN, NN, NK, SN, NH, KN, HN, NQ, HH, RG, KH, RH and SS preferentially bind to guanine. In some embodiments, polypeptide monomers having RVDs RN, NK, NQ, HH, KH, RH, SS and SN preferentially bind to guanine. In some embodiments, polypeptide monomers having RVDs HH, KH, NH, NK, NQ, RH, RN and SS preferentially bind to guanine. In some embodiments, the RVDs that have high binding specificity for guanine are RN, NH RH and KH. In some embodiments, polypeptide monomers having an RVD of NV preferentially bind to adenine and guanine as do monomers having the RVD HN. Monomers having an RVD of NC preferentially bind to adenine, guanine and cytosine, and monomers having an RVD of S (or S*), bind to adenine, guanine, cytosine and thymine with comparable affinity. In more embodiments, monomers having RVDs of H*, HA, KA, N*, NA, NC, NS, RA, and S* bind to adenine, guanine, cytosine and thymine with comparable affinity. Such polypeptide monomers allow for the generation of degenerative TALEs able to bind to a repertoire of related, but not identical, target nucleic acid sequences.

In certain embodiments, the TALE polypeptide has a nucleic acid binding domain containing polypeptide monomers arranged in a predetermined N-terminus to C-terminus order such that each polypeptide monomer binds to a nucleotide of a predetermined target nucleic acid sequence, and where at least one of the polypeptide monomers has an RVD of HN or NH and preferentially binds to guanine, an RVD of NV and preferentially binds to adenine and guanine, an RVD of NC and preferentially binds to adenine, guanine and cytosine or an RVD of S and binds to adenine, guanine, cytosine and thymine.

In some embodiments, each polypeptide monomer of the nucleic acid binding domain that binds to adenine has an RVD of NI, NN, NV, NC or S.

In certain embodiments, each polypeptide monomer of the nucleic acid binding domain that binds to guanine has an RVD of HN, NH, NN, NV, NC or S.

In certain embodiments, each polypeptide monomer of the nucleic acid binding domain that binds to cytosine has an RVD of HD, NC or S.

In some embodiments, each polypeptide monomer that binds to thymine has an RVD of NG or S.

In some embodiments, each polypeptide monomer of the nucleic acid binding domain that binds to adenine has an RVD of NI.

In certain embodiments, each polypeptide monomer of the nucleic acid binding domain that binds to guanine has an RVD of HN or NH.

In certain embodiments, each polypeptide monomer of the nucleic acid binding domain that binds to cytosine has an RVD of HD.

In some embodiments, each polypeptide monomer that binds to thymine has an RVD of NG.

In certain embodiments, the RVDs that have a specificity for adenine are NI, RI, KI, HI, and SI.

In certain embodiments, the RVDs that have a specificity for adenine are HN, SI and RI, most preferably the RVD for adenine specificity is SI.

In certain embodiments, the RVDs that have a specificity for thymine are NG, HG, RG and KG.

In certain embodiments, the RVDs that have a specificity for thymine are KG, HG and RG, most preferably the RVD for thymine specificity is KG or RG.

In certain embodiments, the RVDs that have a specificity for cytosine are HD, ND, KD, RD, HH, YG and SD.

In certain embodiments, the RVDs that have a specificity for cytosine are SD and RD.

FIG. 4B of WO 2012/067428 provides representative RVDs and the nucleotides they target, the entire content of which is hereby incorporated herein by reference.

In certain embodiments, the variant TALE monomers may comprise any of the RVDs that exhibit specificity for a nucleotide as depicted in FIG. 4A of WO2012/067428. All such TALE monomers allow for the generation of degenerative TALEs able to bind to a repertoire of related, but not identical, target nucleic acid sequences.

In certain embodiments, the RVD SH may have a specificity for G, the RVD IS may have a specificity for A, and the RVD IG may have a specificity for T.

In certain embodiments, the RVD NT may bind to G and A. In certain embodiments, the RVD NP may bind to A, T and C. In certain embodiments, at least one selected RVD may be NI, HD, NG, NN, KN, RN, NH, NQ, SS, SN, NK, KH, RH, HH, KI, HI, RI, SI, KG, HG, RG, SD, ND, KD, RD, YG, HN, NV, NS, HA, S*, N*, KA, H*, RA, NA or NC.

The predetermined N-terminal to C-terminal order of the one or more polypeptide monomers of the nucleic acid or DNA binding domain determines the corresponding predetermined target nucleic acid sequence to which the TALE or polypeptides of the invention may bind.

As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8 of WO 2012/067428). Therefore, it follows that the length of the nucleic acid or DNA being targeted is equal to the number of full monomers plus two (see FIG. 44 of WO 2012/067428).

In certain embodiments, nucleic acid binding domains are engineered to contain 5, 6, 7, 8, 9, 10, 11, 12, 13, 14, 15, 16, 17, 18, 19, 20, 21, 22, 23, 24, 25, or more polypeptide monomers arranged in a N-terminal to C-terminal direction to bind to a predetermined 5, 6, 7, 8, 9, 10, 11, 12, 13, 14, 15, 16, 17, 18, 19, 20, 21, 22, 23, 24, 25 nucleotide length nucleic acid sequence.

In certain embodiments, nucleic acid binding domains are engineered to contain 5, 6, 7, 8, 9, 10, 11, 12, 13, 14, 15, 16, 17, 18, 19, 20, 21, 22, 23, 24, 25, 26 or more full length polypeptide monomers that are specifically ordered or arranged to target nucleic acid sequences of length 7, 8, 9, 10, 11, 12, 13, 14, 15, 16, 17, 18, 19, 20, 21, 22, 23, 24, 25, 26, 27 and 28 nucleotides, respectively. In certain embodiments, the polypeptide monomers are contiguous. In some embodiments, half-monomers may be used in the place of one or more monomers, particularly if they are present at the C-terminus of the TALE.

Polypeptide monomers are generally 33, 34 or 35 amino acids in length. With the exception of the RVD, the amino acid sequences of polypeptide monomers are highly conserved or as described herein, the amino acids in a polypeptide monomer, with the exception of the RVD, exhibit patterns that effect TALE activity, the identification of which may be used in preferred embodiments of the invention.

In certain embodiments, when the DNA binding domain may comprise (X1-11-X12X13-X14-33 or 34 or 35)z, wherein X1-11 is a chain of 11 contiguous amino acids, wherein X12X13 is a repeat variable diresidue (RVD), wherein X14-33 or 34 or 35 is a chain of 21, 22 or 23 contiguous amino acids, wherein z is at least 5 to 26, then the preferred combinations of amino acids are LTLD (SEQ ID NO:19936) or LTLA (SEQ ID NO:19937) or LTQV (SEQ ID NO:19938) at X1-4, or EQHG (SEQ ID NO:19939) or RDHG (SEQ ID NO:19940) at positions X30-33 or X31-34 or X32-35. Furthermore, other amino acid combinations of interest in the monomers are LTPD (SEQ ID NO:19941) at X1-4 and NQALE (SEQ ID NO:19942) at XI 6-20 and DHG at X32-34 when the monomer is 34 amino acids in length. When the monomer is 33 or 35 amino acids long, then the corresponding shift occurs in the positions of the contiguous amino acids NQALE (SEQ ID NO:19942) and DHG. In certain embodiments, NQALE (SEQ ID NO:19942) is at X15-19 or X17-21 and DHG is at X31-33 or X33-35.

In certain embodiments, amino acid combinations of interest in the monomers, are LTPD at X1-4 and KRALE (SEQ ID NO:19943) at X16-20 and AHG at X32-34 or LTPE (SEQ ID NO:19944) at X1-4 and KRALE (SEQ ID NO:19943) at XI 6-20 and DHG at X32-34 when the monomer is 34 amino acids in length. When the monomer is 33 or 35 amino acids long, the corresponding shift occurs in the positions of the contiguous amino acids KRALE (SEQ ID NO:19943), AHG and DHG. In certain embodiments, the positions of the contiguous amino acids may be (LTPD at X1-4 and KRALE (SEQ ID NO:19943) at X15-19 and AHG at X31-33) or (LTPE at X1-4 and KRALE (SEQ ID NO:19943) at X15-19 and DHG at X31-33) or (LTPD at X1-4 and KRALE at X17-21 and AHG at X33-35) or (LTPE at X1-4 and KRALE (SEQ ID NO:19943) at X17-21 and DHG at X33-35).

In certain embodiments, contiguous amino acids [NGKQALE](SEQ ID NO:19945) are present at positions X14-20 or X13-19 or X15-21. These representative positions put forward various embodiments of the invention and provide guidance to identify additional amino acids of interest or combinations of amino acids of interest in all the TALE monomers (see FIGS. 24A-24F, and 25 of WO 2012/067428).

Exemplary amino acid sequences of conserved portions of polypeptide monomers are provided below. The position of the RVD in each sequence is represented by XX or by X* (wherein (*) indicates that the RVD is a single amino acid and residue 13 (X13) is absent).

(SEQ ID NO: 19402)
LTPAQVVAIASXXGGKQALETVQRLLPVLCQDHG
(SEQ ID NO: 19403)
LTPAQVVAIASX*GGKQALETVQRLLPVLCQDHG
(SEQ ID NO: 19404)
LTPDQVVAIANXXGGKQALATVQRLLPVLCQDHG
(SEQ ID NO: 19405)
LTPDQVVAIANXXGGKQALETLQRLLPVLCQDHG
(SEQ ID NO: 19406)
LTPDQVVAIANXXGGKQALETVQRLLPVLCQDHG
(SEQ ID NO: 19407)
LTPDQVVAIASXXGGKQALATVQRLLPVLCQDHG
(SEQ ID NO: 19408)
LTPDQVVAIASXXGGKQALETVQRLLPVLCQDHG
(SEQ ID NO: 19409)
LTPDQVVAIASXXGGKQALETVQRVLPVLCQDHG
(SEQ ID NO: 19410)
LTPEQVVAIASXXGGKQALETVQRLLPVLCQAHG
(SEQ ID NO: 19411)
LTPYQVVAIASXXGSKQALETVQRLLPVLCQDHG
(SEQ ID NO: 19412)
LTREQVVAIASXXGGKQALETVQRLLPVLCQDHG
(SEQ ID NO: 19413)
LSTAQVVAIASXXGGKQALEGIGEQLLKLRTAPYG
(SEQ ID NO: 19414)
LSTAQVVAVASXXGGKPALEAVRAQLLALRAAPYG

A further listing of TALE monomers excluding the RVDs which may be denoted in a sequence (X1-11-X14-34 or X1-11-X14-35), wherein X is any amino acid and the subscript is the amino acid position is provided in FIG. 24A-F of WO 2012/067428, which is incorporated herein by reference.

In certain embodiments, TALE polypeptide binding efficiency is increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 19415)
MDPIRSRTPSPARELLSGPQPDGVQPTADRGVSPPAGGPLDGLPARRTMS
RTRLPSPPAPSPAFSADSFSDLLRQFDPSLENTSLFDSLPPFGAHHTEAA
TGEWDEVQSGLRAADAPPPTMRVAVTAARPPRAKPAPRRRAAQPSDASPA
AQVDLRTLGYSQQQQEKIKPKVRSTVAQHHEALVGHGFTHAHIVALSQHP
AALGTVAVKYQDMIAALPEATHEAIVGVGKQWSGARALEALLTVAGELRG
PPLQLDTGQLLKIAKRGGVTAVEAVHAWRNALTGAPLN

An exemplary amino acid sequence of a C-terminal capping region is:

(SEQ ID NO: 19416)
RPALESIVAQLSRPDPALAALTNDHLVALACLGGRPALDAVKKGLPHAPA
LIKRTNRRIPERTSHRVADHAQVVRVLGFFQCHSHPAQAFDDAMTQFGMS
RHGLLQLFRRVGVTELEARSGTLPPASQRWDRILQASGMKRAKPSPTSTQ
TPDQASLHAFADSLERDLDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE (including TALEs) polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. In certain embodiments, C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result. % homology may be calculated over contiguous sequences, i.e., one sequence is aligned with the other sequence and each amino acid or nucleotide in one sequence is directly compared with the corresponding amino acid or nucleotide in the other sequence, one residue at a time. This is called an “ungapped” alignment. Typically, such ungapped alignments are performed only over a relatively short number of residues.

Additional sequences for the conserved portions of polypeptide monomers and for N-terminal and C-terminal capping regions are included in the sequences with the following gene accession numbers: AAW59491.1, AAQ79773.2, YP_450163.1, YP_001912778.1, ZP_02242672.1, AAW59493.1, AAY54170.1, ZP_02245314.1, ZP_02243372.1, AAT46123.1, AAW59492.1, YP 451030.1, YP 001915105.1, ZP_02242534.1, AAW77510.1, ACD11364.1, ZP 02245056.1, ZP_02245055.1, ZP 02242539.1, ZP 02241531.1, ZP_02243779.1, AAN01357.1, ZP 02245177.1, ZP 02243366.1, ZP_02241530.1, AAS58130.3, ZP 02242537.1, YP 200918.1, YP_200770.1, YP 451187.1, YP_451156.1, AAS58127.2, YP_451027.1, UR_451025.1, AAA92974.1, UR_001913755.1, ABB70183.1, UR_451893.1, UR_450167.1, ABY60855.1, UR_200767.1, ZR 02245186.1, ZR_02242931.1 ZR_02242535.1, AAU54169.1, UR 450165.1, UR_001913452.1, AAS58129.3, ACM44927.1, ZR_02244836.1, AAT46125.1, UR_450161.1 ZR_02242546.1, AAT46122.1, UR_451897.1, AAF98343.1, UR_001913484.1, AAY54166.1, UR_001915093.1, UR_001913457.1, ZR_02242538.1, UR_200766.1, UR_453043.1, UR_001915089.1, UR_001912981.1, ZR_02242929.1, UR_001911730.1, UR_201654.1, UR_199877.1, ABB70129.1, UR_451696.1, UR_199876.1, AAS75145.1, AAT46124.1, UR_200914.1, UR 001915101.1, ZR_02242540.1, AAG02079.2, UR_451895.1, YP 451189.1, UR_200915.1, AAS46027.1, UR_001913759.1, UR_001912987.1, AAS58128.2, AAS46026.1, UR_201653.1, UR 202894.1, UR 001913480.1, ZR_02242666.1, R_001912775.1, ZR_02242662.1, AAS46025.1, AAC43587.1, BAA37119.1, NPJ544725.1, AB077779.1, BAA37120.1, ACZ62652.1, BAF46271.1, ACZ62653.1, NPJ544793.1, AB077780.1, ZR 022437401 ZR_02242930.1, AAB69865.1, AAY54168.1, ZR_02245191.1, UR_001915097.1, ZR_02241539.1, UR_451158.1, BAA37121.1, UR_001913182.1, UR_200903.1, ZR_02242528.1, ZR 06705357.1, ZR_06706392.1, AD148328.1, ZR 06731493.1, AD148327.1, AB077782.1, ZR 06731656.1, NR_942641.1, AAY43360.1, ZR_06730254.1, ACN39605.1, UR_451894.1, UR 201652.1, UR_001965982.1, BAF46269.1, NPJ544708.1, ACN82432.1, AB077781.1, P14727.2, BAF46272.1, AAY43359.1, BAF46270.1, NR_644743.1, ABG37631.1, AAB00675.1, YP 199878.1, ZR_02242536.1, CAA48680.1, ADM80412.1, AAA27592.1, ABG37632.1, ABP97430.1, ZR_06733167.1, AAY43358.1, 2KQ5_A, BAD42396.1, AB027075.1, UR 002253357.1, UR_002252977.1, AB027074.1, AB027067.1, AB027072.1, AB027068.1, UR_003750492.1, AB027073.1, NR_519936.1, AB027071.1, AB027070.1, and AB027069.1, each of which is hereby incorporated by reference.

In some embodiments, the TALEs described herein also include a nuclear localization signal and/or cellular uptake signal. Such signals are known in the art and may target a TALE to the nucleus and/or intracellular compartment of a cell. Such cellular uptake signals include, but are not limited to, the minimal Tat protein transduction domain which spans residues 47-57 of the human immunodeficiency virus Tat protein: YGRKKRRQRRR (SEQ ID NO:19952).

In some embodiments, the TALEs described herein include a nucleic acid or DNA binding domain that is a non-TALE nucleic acid or a non-TALE DNA binding domain.

As used herein the term “non-TALE DNA binding domain” refers to a DNA binding domain that has a nucleic acid sequence corresponding to a nucleic acid sequence which is not substantially homologous to a nucleic acid that encodes for a TALE protein or fragment thereof, e.g., a nucleic acid sequence which is different from a nucleic acid that encodes for a TALE protein and which is derived from the same or a different organism.

In certain embodiments, the TALEs described herein include a nucleic acid or DNA binding domain that is linked to a non-TALE polypeptide.

A “non-TALE polypeptide” refers to a polypeptide having an amino acid sequence corresponding to a protein which is not substantially homologous to a TALE protein or fragment thereof, e.g., a protein which is different from a TALE protein and which is derived from the same or a different organism. In this context, the term “linked” is intended include any manner by which the nucleic acid binding domain and the non-TALE polypeptide could be connected to each other, including, for example, through peptide bonds by being part of the same polypeptide chain or through other covalent interactions, such as a chemical linker. The non-TALE polypeptide may be linked, for example to the N-terminus and/or C-terminus of the nucleic acid binding domain, may be linked to a C-terminal or N-terminal cap region, or may be connected to the nucleic acid binding domain indirectly.

In certain embodiments, the TALEs or polypeptides of the invention comprise chimeric DNA binding domains. Chimeric DNA binding domains may be generated by fusing a full TALE (including the N- and C-terminal capping regions) with another TALE or non-TALE DNA binding domain such as zinc finger (ZF), helix-loop-helix, or catalytically-inactivated DNA endonucleases (e.g., EcoRI, meganucleases, etc.), or parts of TALE may be fused to other DNA binding domains. The chimeric domain may have novel DNA binding specificity that combines the specificity of both domains.

In certain embodiments, the TALE polypeptides of the invention include a nucleic acid binding domain linked to the one or more effector domains. In certain embodiments, the effector domain is a nickase or nuclease.

In certain embodiments, the sequence-specific nuclease is a zinc finger nuclease (ZFN), such as an artificial zinc-finger nuclease having arrays of zinc-finger (ZF) modules to target new DNA-binding sites in a target sequence (e.g., target sequence or target site in the genome). Each zinc finger module in a ZF array targets three DNA bases. A customized array of individual zinc finger domains is assembled into a ZF protein (ZFP). The resulting ZFP can be linked to a functional domain such as a nuclease.

ZF nucleases (ZFN) may be used as alternative programmable nucleases for use in retron-based editing in place of RNA-guide nucleases. ZFN proteins have been extensively described in the art, for example, in Carroll et al., “Genome Engineering with Zinc-Finger Nucleases,” Genetics, August 2011, Vol. 188: 773-782; Durai et al., “Zinc finger nucleases: custom-designed molecular scissors for genome engineering of plant and mammalian cells,” Nucleic Acids Res, 2005, Vol. 33: 5978-90; and Gaj et al., “ZFN, TALEN, and CRISPR/Cas-based methods for genome engineering,” Trends Biotechnol. 2013, Vol. 31: 397-405, each of which are incorporated herein by reference in their entireties.

In certain embodiments, the ZF-linked nuclease is a catalytic domain of the Type IIS restriction enzyme FokI (see Kim et al., PNAS U.S.A. 91:883-887, 1994; Kim et al., PNAS U.S.A. 93:1156-1160, 1996, both incorporated herein by reference).

In certain embodiments, the ZFN comprises paired ZFN heterodimers, resulting in increased cleavage specificity and/or decreased off-target activity. In this embodiment, each ZFN in the heterodimer targets different nucleotide sequences separated by a short spacer (see Doyon et al., Nat. Methods 8:74-79, 2011, incorporated herein by reference).

In certain embodiments, the ZFN comprises a polynucleotide-binding domain (comprising multiple sequence-specific ZF modules) and a polynucleotide cleavage nickase domain.

In certain embodiments, the ZFs are engineered using libraries of two finger modules.

In certain embodiments, strings of two-finger units are used in ZFNs to improve DNA binding specificity from polyzinc finger peptides (see PNAS USA 98: 1437-1441, incorporated herein by reference).

In certain embodiments, the ZFN has more than 3 fingers. In certain embodiments, the ZFN has 4, 5, or 6 fingers. In certain embodiments, the ZF modules in the ZFN are separated by one or more linkers to improve specificity.

In certain embodiments, the ZF of the ZFN includes substitutions in the dimer interface of the cleavage domain that prevent homodimerization between ZFs, but allow heterodimers to form.

In certain embodiments, the ZF of the ZFN has a design that retains activity while suppressing homodimerization.

In certain embodiments, the ZFN is any one of the ZF nucleases in Table 1 of Carroll et al., Genetics 188(4):773-782, 2011, incorporated herein by reference.

General principles and guidance for generating ZF, ZF arrays, and ZFN can be found in the art, such as the modular design (where the different modules can be rearranged and assembled into new combinations for new targets) of the ZF or ZF arrays in the ZFN as taught in Carroll et al., Nat. Protoc. 1: 1329-1341, 2006 (incorporated herein by reference); the new three-finger sets for engineered ZFs generated by using partially randomized libraries; profiling the DNA-binding specificities of engineered Cys2His2 zinc finger domains using a rapid cell-based method (see Nucleic Acids Res. 35: e81, incorporated by reference). ZFs for certain DNA triplets that work well in neighbor combination are described in Sander et al., 2011. Selection-free zinc-finger-nuclease engineering by context-dependent assembly (CoDA) is taught in Nat. Methods 8: 67-69). ToolGen describes the individual fingers in their collection that are best behaved in modular assembly (Kim et al., 2011). Preassembled zinc-finger arrays for rapid construction of ZFNs are taught in Nat. Methods 8:7.

Additional, non-limiting ZFs and AFNz that can be adapted for use in the instant invention include those described in WO2010/065123, WO2000/041566, WO2003/080809, WO2015/143046, WO2016/183298, WO2013/044008, WO2015/031619, WO2017/136049, WO2016/014794, WO2017/091512, WO1995/009233, WO2000/023464, WO2000/042219, WO2002/026960, WO2001/083793; U.S. Pat. Nos. 9,428,756, 9,145,565, 8,846,578, 8,524,874, 6,777,185, 6,599,692, 7,235,354, 6,503,717, 7,491,531, 7,943,553, 7,262,054, 8,680,021, 7,705,139, 7,273,923, 6,780,590, 6,785,613, 7,788,044, 7,177,766, 6,453,242, 6,794,136, 7,358,085, 8,383,766, 7,030,215, 7,013,219, 7,361,635, 7,939,327, 8,772,453, 9,163,245, 7,045,304, 8,313,925, 9,260,726, 6,689,558, 8,466,267, 7,253,273, 7,947,873, 9,388,426, 8,153,399, 8,569,253, 8,524,221, 7,951,925, 9,115,409, 8,772,008, 9,121,072, 9,624,498, 6,979,539, 9,491,934, 6,933,113, 9,567,609, 7,070,934, 9,624,509, 8,735,153, 9,567,573, 6,919,204, US2002-0081614, US2004-0203064, US2006-0166263, US2006-0292621, US2003-0134318, US2006-0294617, US2007-0287189, US2007-0065931, US2003-0105593, US2003-0108880, US2009-0305402, US2008-0209587, US2013-0123484, US2004-0091991, US2009-0305977, US2008-0233641, US2014-0287500, US2011-0287512, US2009-0258363, US2013-0244332, US2007-0134796, US2010-0256221, US2005-0267061, US2012-0204282, US2012-0252122, US2010-0311124, US2016-0215298, US2008-0031109, US2014-0017214, US2015-0267205, US2004-0235002, US2004-0204345, US2015-0064789, US2006-0063231, US2011-0265198, US2017-0218349, all incorporated herein by reference.

Polynucleotides and vectors capable of expressing one or more of the ZFNs are also provided herein, which can be part of the vector system of the invention. The polynucleotides and vectors can be expressed in a cell, such as a eukaryotic cell, a mammalian cell, or a human cell. Suitable vectors, cells and expression systems are described in greater detail elsewhere herein, and can be suitable for use with the TALEs, the meganucleases, and the CRISPR-Cas nucleases.

In certain embodiments, the sequence-specific nuclease is a meganuclease.

Meganucleases are a class of sequence-specific endonucleases that recognize large DNA target sites (>12 bp). These proteins can cleave a unique chromosomal sequence without affecting overall genome integrity. Meganucleases create site specific DNA DSBs, and, in the presence of donor DNA, such as one present in the heterologous nucleic acid encompassed by or encoded by the engineered retron of the invention, promotes the integration of the donor DNA at the cleavage site through homologous recombination (HR).

In certain embodiments, the meganuclease is a homing endonuclease, which is a widespread class of proteins found in eukaryotes, bacteria and archaea. In certain embodiments, the meganuclease is of the LAGLIDADG (SEQ ID NO:19953) family of homing endonucleases.

In certain embodiments, the meganuclease is I-Scel, I-Cre-I, I-Dmol, or an engineered or a naturally occurring variant thereof. The hallmark of these proteins is a well conserved LAGLIDADG (SEQ ID NO:19953) peptide motif, termed (do)decapeptide, found in one or two copies. Homing endonucleases with only one such motif, such as I-Crel or I-Ceul, function as homodimers. In contrast, larger proteins bearing two (do)decapeptide motifs, such as I-Scel, Pl-Scel and I-Dmol are single chain proteins.

Additional homing nucleases are found at the website of homingendonuclease.net, which provides a database listing basic properties of known LAGLIDADG (SEQ ID NO:19953) homing endonucleases. See also Taylor et al., Nucleic Acids Research 40 (W1): W110-W116, 2012 (all incorporated herein by reference).

In certain embodiments, specificity (or polynucleotide recognition) of the meganuclease is modified by altering the amino acids within the meganuclease, and/or by fusing other effector domains with the meganuclease.

In certain embodiments, the meganuclease is a megaTAL, which includes a DNA binding domain from a TALE.

In certain embodiments, the meganuclease is engineered to have nickase activity.

Additional suitable natural and engineered meganucleases and megaTALs are described in WO2006/097853, WO2004/067736, WO2012/030747, WO2007/123636, WO2010/001189, WO2018/071565, WO2007/049095, WO2009/068937, WO2005/105989, WO2008/102198, WO2007/057781, WO2019/126558, WO2010/046786, US2010-0151556, US2014-0121115, US2011-0207199, US2012-0301456, US2013-0189759, US2011-0158974, US2010-0144012, US2014-0112904, US2013-0196320, US2010-0203031, US2010-0167357, US2012-0272348, US2012-0258537, US2011-0072527, US2013-0183282, US2014-0178942, US2012-0260356, US2013-0236946, US2010-0325745, US2011-0041194, US2014-0004608, US2011-0263028, US2011-0225664, US2013-0145487, US2013-0045539, US2012-0171191, US2015-0315557, US2014-0017731, US2011-0091441, US2014-0038239, US2010-0229252, US2009-0222937, US2010-0146651, US2013-0059387, US2011-0179507, US2013-0326644, US2006-0078552, US2004-0002092, US2012-0052582, US2009-0162937, US2010-0086533, US2009-0220476, U.S. Pat. Nos. 8,802,437, 7,842,489, 8,715,992, 8,426,177, 8,476,072, 9,365,864, 9,540,623, 9,273,296, 9,290,748, 8,163,514, 8,148,098, 8,143,016, 8,143,015, 8,133,697, 8,129,134, 8,124,369, 8,119,361, 7,897,372, 9,683,257, 10,287,626, 10,273,524, 10,000,746, 10,006,052, 7,919,605, 9,018,364, 10,407,672, 8,211,685, 9,365,864, 7,476,500, all incorporated herein by reference.

In certain embodiments, the sequence-specific nuclease is TnpB, which is a programmable RNA-guided DNA endonuclease. It is believed that TnpB is a functional progenitor of the CRISPR-Cas nucleases.

Transposons are mobile genetic elements that contain only the genes required for their transposition and its regulation. These elements encode the tnpA transposase, which is essential for mobilization, and often carry an accessory tnpB gene, which is dispensable for transposition. TnpB has been shown to be a nuclease that is guided by an RNA, derived from the right-end element of a transposon, to cleave DNA next to the 5′-TTGAT transposon-associated motif, and TnpB can be reprogrammed to cleave DNA target sites in human cells.

In certain embodiments, TnpB is from D. radiodurans ISDra2 of the IS200/1S605 family.

In certain embodiments, TnpB is from transposon PsiTn554.

In certain embodiments, the sequence-specific nuclease is a TnpB-like protein, such as Fanzor1 or Fanzor2. These proteins are widespread in diverse eukaryotic transposable elements (TEs), and in large double-stranded DNA (dsDNA) viruses infecting eukaryotes. Fanzor and TnpB proteins share the same conserved amino acid motif in their C-terminal half regions: D-X(125, 275)-[TS]-[TS]-X-X-[C4 zinc finger]-X(5,50)-RD, but are highly variable in their N-terminal regions. Fanzor1 proteins are frequently captured by DNA transposons from different superfamilies including Helitron, Mariner, IS4-like, Sola and MuDr. In contrast, Fanzor2 proteins appear only in some IS607-type elements.

In certain embodiments, the sequence-specific nuclease is IscB.

ISC (insertion sequences Cas9-like) is a novel group of bacterial and archaeal DNA transposons that encode Cas9 homologs. The ISC transposon-encoded two nuclease domain-containing proteins are the likely ancestors of the CRISPR-associated Cas9. The homology region includes the arginine-rich helix and the HNH nuclease domain that is inserted into the RuvC-like nuclease domain. ISC genes, however, are not linked to Cas genes or CRISPR. They represent a distinct group of nonautonomous transposons, with many diverse families of full-length ISC transposons. Their terminal sequences (particularly 3′ termini) are similar to those of IS605 superfamily transposons that are mobilized by the Y1 tyrosine transposase encoded by the TnpA gene, and often also encode the TnpB protein containing the RuvC-like endonuclease domain. The terminal regions of the ISC and IS605 transposons contain palindromic structures that are likely recognized by the Y1 transposase. The transposons from these two groups are inserted either exactly in the middle or upstream of specific 4-bp target sites, without target site duplication.

In certain embodiments, the sequence-specific nuclease is a restriction endonuclease (RE), such as an RE with stringent/long recognition sequence of at least 8 nts.

In certain embodiments, the RE is a rare-cutter RE with seven and eight base pair recognition sequences. Exemplary rare-cutter RE enzyme include NotI, which cuts after the first GC of a 5′-GCGGCCGC-3′ sequence (SEQ ID NO: 19417).

In certain embodiments, the components of the system—e.g., the retron encoded ncRNA or msDNA in complex with the RT, the sequence-specific nuclease, and the DNA-repair modulating biomolecule, may form multiple complexes in a so-called split complex configuration. The multiple complexes may be brought together to form a functional complex.

For example, in some embodiments, a first component in the system may be a split protein or domain. A fragment of the split protein or domain may associate with a second component of the system, while another fragment of the split protein or domain may associate with a third component of the system. The two fragments of the split protein or domain may be brought together (e.g., along with the other components of the system) to form a functional complex.

In certain embodiments, the split protein or domain is the sequence-specific nuclease, e.g., a CRISPR/Cas effector enzyme (e.g., Cas protein such as Cas9 or Cas12), a ZFN, a TALEN, a meganuclease, TnpB, IscB, or a restriction endonuclease (RE).

In certain embodiments, the split protein or domain is the reverse transcriptase domain.

In certain embodiments, the split protein or domain is the DNA-repair modulating biomolecule.

For example a first fragment of the sequence-specific nuclease may associate with the reverse transcriptase domain and a second fragment of the sequence-specific nuclease may associate with the DNA-repair modulating biomolecule. The two fragments of the split protein or domain may be brought together (e.g., along with the reverse transcriptase domain and the DNA-repair modulating biomolecule) to form a functional complex. The associations between the parts of the split protein or domain may be through adaptor proteins or linkers described herein (e.g., those used for associating Cas proteins with function domains).

In certain embodiments, the split protein or domain is split in the sense that the two parts of the split protein or domain substantially comprise a functioning split protein or domain. Ideally, the split should always be so that the catalytic domain(s) are unaffected. That split protein or domain may function as a sequence-specific nuclease or it may be a dead-Cas which is essentially an RNA-binding protein with very little or no catalytic activity, due to typically mutation(s) in its catalytic domains.

Each fragment of the split protein or domain may be fused to a dimerization partner. For example, rapamycin sensitive dimerization domains enables a chemically inducible split protein or domain for temporal control of the split protein or domain's activity. The split protein or domain can thus be rendered chemically inducible by being split into two fragments and that rapamycin-sensitive dimerization domains may be used for controlled reassembly of the split protein or domain. The two parts of the split protein or domain can be thought of as the N′ terminal part and the C′ terminal part of the split protein or domain. The fusion is typically at the split point of the split protein or domain. In other words, the C′ terminal of the N′ terminal part of the split protein or domain is fused to one of the dimer halves, whilst the N′ terminal of the C′ terminal part is fused to the other dimer half.

The split protein or domain does not have to be split in the sense that the break is newly created. The split point is typically designed in silico and cloned into the constructs. Together, the two parts of the split protein or domain, the N‘ terminal and C’ terminal parts, form a full split protein or domain, comprising preferably at least 70% or more of the wildtype amino acids (or nucleotides encoding them), at least 80% or more, at least 90% or more, at least 95% or more, and at least 99% or more of the wildtype amino acids (or nucleotides encoding them). When the two parts are brought together, the desired split protein or domain function is restored or reconstituted. The dimer may be a homodimer or a heterodimer.

In certain embodiments, the protein components of the system—e.g., the RT, the sequence-specific nuclease (a CRISPR/Cas effector enzyme, a ZFN, a TALEN, a meganuclease, TnpB, IscB, or a restriction endonuclease (RE)), and the DNA-repair modulating biomolecule, may further comprise one or more additional functional domains.

In certain embodiments, the functional domain comprises a nuclear localization signal (NLS). In certain embodiments, one or more C-terminal or N-terminal NLSs are attached. In certain embodiments, a C-terminal NLS is attached for expression and nuclear targeting in eukaryotic cells, e.g., human cells. In certain embodiments, the NLS(s) may be at a location that is not at the C-terminus or N-terminus, for example, the NLS(s) may be between two polypeptides.

Non-limiting examples of NLSs include an NLS sequence derived from: the NLS of the SV40 virus large T-antigen; the NLS from nucleoplasmin (e.g., the nucleoplasmin bipartite NLS); the c-myc NLS; the hRNPAl M9 NLS; the NLS of the IBB domain from importin-alpha; the NLS of the myoma T protein; the NLS of human p53; the NLS of mouse c-abl IV; the NLS of the influenza virus NS1; the NLS of the Hepatitis virus delta antigen; the NLS of the mouse Mxl protein; the NLS of the human poly(ADP-ribose) polymerase; and the NLS of the steroid hormone receptors (human) glucocorticoid. Exemplary NLS sequences include those described in paragraph [00106] of Feng Zhang et al. (WO2016/106236), all incorporated herein by reference.

In certain embodiments, the functional domain comprises at least two NLS domains. The one or more NLS domain(s) may be positioned at or near or in proximity to a terminus of a polypeptide and, if two or more NLSs, each of the two may be positioned at or near or in proximity to a terminus of the polypeptide.

In any of the fusion proteins, the fusion between the two domains (such as the RT and Cas enzyme, or the Cas and the DNA-repair modulating biomolecule) may be linked through a linker.

A “linker” as used herein includes a peptide which joins two proteins or domains to form a fusion protein. Generally, such molecules have no specific biological activity other than to join or to preserve some minimum distance or other spatial relationship between the proteins/domains. However, in certain embodiments, the linker may be selected to influence some property of the linker and/or the fusion protein such as the folding, net charge, or hydrophobicity of the linker. Suitable linkers for use in the present disclosure are well-known to those of skill in the art and include, but are not limited to, straight or branched-chain carbon linkers, heterocyclic carbon linkers, or peptide linkers. However, as used herein, the linker may also be a covalent bond (carbon-carbon bond or carbon-heteroatom bond).

In particular embodiments, the linker is used to separate the sequence-specific nuclease (a CRISPR/Cas effector enzyme, a ZFN, a TALEN, a meganuclease, TnpB, IscB, or a restriction endonuclease (RE)) and the RT and/or the DNA-repair modulating biomolecule by a distance sufficient to ensure that each protein domain retains its required functional property.

Preferred peptide linker sequences adopt a flexible extended conformation and do not exhibit a propensity for developing an ordered secondary structure. In certain embodiments, the linker can be a chemical moiety which can be monomeric, dimeric, multimeric or polymeric. In certain embodiments, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence.

In certain embodiments, the linker comprises a GlySer-rich sequence, such as a GₙS linker (n=1, 2, 3, 4, or 5, such as GS or G₄S) (SEQ ID NO:19946), or repeats thereof (such as 1, 2, 3, 4, 5, 6, 7, 8, 9, 10, 11, 12, 13, 14, 15, 16, 17, 18, 19, or 20 repeats, optionally with an overall length of about 4-30 residues, 4-20 residues, or 4-10 residues).

In certain embodiments, the linker comprises a G₄S linker with 3, 6, 9, or 12 repeats.

In certain embodiments, the linker is one disclosed in Maratea et al., Gene 40: 39-46, 1985; Murphy et al., PNAS USA 83: 8258-62, 1986; U.S. Pat. No. 4,935,233; or 4,751,180, all incorporated by reference.

In certain embodiments, the linker comprises a GlySer linker such as GGS, GGGS (SEQ ID NO:19947), GSG, GGGGS (SEQ ID NO:19948), optionally with repeats of 3 (such as (GGS)₃, (SEQ ID NO:19949) (GGGGS)₃) (SEQ ID NO:19950), 4, 5, 6, 7, 8, 9, 10, 11, or 12 or more, to provide suitable lengths.

In certain embodiments, the linker comprises (GGGGS)₃₋₁₅ (SEQ ID NO:19951), such as (GGGGS)₃₋₁₁ (SEQ ID NO:19951), e.g., GGGGS (SEQ ID NO:19951) with 1, 2, 3, 4, 5, 6, 7, 8, 9, 10, or 11 repeats.

In certain embodiments, the linker comprises LEPGEKPYKCPECGKSFSQSGALTRHQRTHTR (SEQ ID NO: 19418).

In yet an additional embodiment, the linker is an XTEN linker.

In certain embodiments, N- and/or C-terminal NLSs also function as linker (e.g., PKKKRKVEASSPKKRKVEAS (SEQ ID NO: 19420)).

The gRNA and the various nucleases and fusions thereof can be provided in the form of a protein, optionally where the nuclease is complexed with a gRNA, or provided by a nucleic acid encoding the RNA-guided nuclease, such as an RNA (e.g., messenger RNA) or DNA (expression vector). In some embodiments, the RNA-guided nuclease and the gRNA are both provided by vectors. Both can be expressed by a single vector or separately on different vectors. The vectors encoding the RNA-guided nuclease and gRNA may be included in the vector system comprising the engineered retron msr gene, msd gene and ret gene sequences.

Codon usage may be optimized to improve production of the engineered retron e.g., retron reverse transcriptase, ncRNA and/or RNA-guided nuclease in a particular cell or organism. For example, a nucleic acid encoding an ncRNA, RNA-guided nuclease or reverse transcriptase can be modified to substitute codons having a higher frequency of usage in a the particular cell such as a eukaryotic cell (e.g., yeast cell, a human cell, a non-human cell, a mammalian cell, a rodent cell, a mouse cell, a rat cell), or any other host cell of interest, as compared to the naturally occurring polynucleotide sequence. When a nucleic acid encoding the reverse transcriptase or ncRNA is introduced into cells, the protein can be transiently, conditionally, or constitutively expressed in the cell.

### F. RT-PN Fusion Proteins

The recombinant retron-based editing system described herein contemplates fusion proteins comprising a programmable nuclease (PN) and a RT, optionally joined by a linker. The application contemplates any suitable programmable nuclease and RT (e.g., retron RTs of Table A) to be combined in a single fusion protein. In one embodiment, the RT is joined to the N-terminus of the PN. In another embodiment, the RT is joined to the C-terminus of the PN. The Examples of PNs and RTs are each defined herein.

In various embodiments, the fusion proteins may comprise any suitable structural configuration. For example, the fusion protein may comprise from the N-terminus to the C-terminus direction, a PN fused to a RT. In other embodiments, the fusion protein may comprise from the N-terminus to the C-terminus direction, a RT fused to a NP. The fused domain may optionally be joined by a linker, e.g., an amino acid sequence.

### G. Retron-Based Gene Editing Systems

The present disclosure relates to novel genome editing systems that include retrons, including retron RT and ncRNAs. In exemplary embodiments, the editing systems comprise:
(a) one or more retron RT polypeptide sequences comprising at least 45%, 46%, 47%, 48%, 49%, 50%, 51%, 52%, 53%, 54%, 55%, 56%, 57%, 58%, 59%, 60%, 61%, 62%, 63%, 64%, or 65% sequence identity to any one of the amino acid sequences of Table A, or a polynucleotide sequence encoding same (as also provided in Table A);
(b) (a) one or more retron ncRNA polynucleotide sequences comprising at least 45%, 46%, 47%, 48%, 49%, 50%, 51%, 52%, 53%, 54%, 55%, 56%, 57%, 58%, 59%, 60%, 61%, 62%, 63%, 64%, or 65% sequence identity to any one of the amino acid sequences of Table B, wherein in one embodiment the retron RT and ncRNA are cognate pairs;
(c) one or more programmable nucleases (e.g., TnpB, Cas12a, or Cas9 as disclosed herein); and
(d) one or more polynucleotide sequences comprising a guide RNA, wherein the guide RNA comprises a complementary sequence to that of a targeted polynucleotide sequence and which binds to the programmable nuclease.

In other aspects, the retron-based gene editing systems may comprise one or more additional accessory proteins having genome modifying functions, including recombinases, invertases, nucleases, polymerases, ligases, deaminases, reverse transcriptases, or epigenetic modifying functions. In various embodiments, the accessory proteins may be provided separately. In other embodiments, the accessory proteins may be fused to a retron component (e.g., fused to a retron RT), optionally with a linker.

In various embodiments, the genome editing system may comprise a guide RNA, which hybridizes to one or more targeted polynucleotide sequence. In preferred embodiments, the guide RNA of the genome editing system comprises 12-40 nucleotides.

In various embodiments, the genome editing system comprises the targeted polynucleotide sequence comprises one or more protospacer adjacent motif (PAM) recognition domains selected from 5′-TTTN-3′, 5′-TTN-3′, 5′-TNN-3′, 5′-TTV-3′, or 5′-TTTV-3′, wherein N=A, T, C or G and V=A, C or G. In additional embodiments, the targeted polynucleotide sequence comprises one or more relaxed PAM recognition domains. Jacobsen, Thomas et al. “Characterization of Cas12a nucleases reveals diverse PAM profiles between closely-related orthologs.” Nucleic acids research vol. 48,10 (2020): 5624-5638. doi:10.1093/nar/gkaa272. Previous work has demonstrated to address the limitation for the requirement for an extended TTTV protospacer adjacent motif (PAM) by expanding the targeting range for non-canonical PAMs (such as ATTA, CTTA, GTTA, and TCTA) Kleinstiver, Benjamin P et al. “Engineered CRISPR-Cas12a variants with increased activities and improved targeting ranges for gene, epigenetic and base editing.” Nature biotechnology vol. 37,3 (2019): 276-282. doi:10.1038/s41587-018-0011-0. Most of the Cpf1 nucleases require thymine-rich PAM. Different studies have demonstrated an increased Cpf1 targeting range using in vitro and in vivo (E. co/i) PAM identification assays. Zhang, Xiaochun, et al. “Multiplex gene regulation by CRISPR-ddCpf1.” Cell discovery 3.1 (2017): 1-9. The two Cpf1 endonucleases, AsCpf1 and LbCpf1, require TTTV as a PAM sequence, where V can be A, C, or G nucleotides. Mutations at position S542R/K607R and S542R/K548V/N552R produced AsCpf1 variants, and these are able to recognize TYCV and TATV PAMs, respectively, where Y can be C or T. Gao, Linyi, et al. “Engineered Cpf1 variants with altered PAM specificities.” Nature biotechnology 35.8 (2017): 789-792. The AsCpf1 showed increased activity for TTTV PAMs and decreased activity with TTTT PAM Kim, Hui K., et al. “In vivo high-throughput profiling of CRISPR-Cpf1 activity.” Nature methods 14.2 (2017): 153-159.

Accordingly, it is within the scope of the disclosure to devise the editing system to recognize altered PAM recognition domains for genome editing. In preferred embodiments, the programmable polypeptide component recognizes one or more non-canonical PAM sequence in the targeted polynucleotide sequence, the PAM upstream of the crRNA-complementary DNA sequence on the non-target strand. In related embodiments, the gRNA has a seed sequence of eight nucleotides, located at the 5′ end of the spacer, and is proximal to the PAM sequence on the targeted polynucleotide sequence.

In further embodiments, the one or more polypeptide sequences and the one or more polynucleotide sequences comprising a cognate guide RNA of the genome editing system form a ribonucleoprotein complex.

In various embodiments wherein the programmable nuclease is a Type V enzyme (e.g., Cas12a), the one or more polypeptide sequences of the genome editing system comprise:
one or more α-helical recognition lobe (REC) and a nuclease lobe (NUC);
a Wedge (WED), α-helical recognition lobe (REC), PAM-interacting (PI),
RuvC nuclease, Bridge Helix (BH) and NUC domains; or
one or more domains selected from RuvC, REC, WED, BH, PI and NUC domains.

In various embodiments wherein the programmable nuclease is a Type II enzyme (e.g., Cas9), the one or more polypeptide sequences of the genome editing system comprise:
An alpha-helical lobe and
A nuclease lobe that comprises two nuclease domains (the RuvC domain which cleaves the non-target DNA strand and the HNH domain that cleaves the target strand).

In various embodiments, the molecular weight of the programmable nuclease component is characterized in its molecular weight to be about 50 kDa-100 kDa, 100 kDa-200 kDa, 200 kDa-500 kDa.

In additional embodiments, the polypeptide sequences comprise at least one activity selected from endonuclease activity; endoribonuclease activity, or RNA-guided DNase activity. In such embodiments, the cognate guide RNA and the Cas12a protein modifies the targeted polynucleotide sequence of a host cell genome. In certain instances, the targeted polynucleotide sequence is modified by an insertion, deletion or alteration of one or more base pairs at the targeted polynucleotide sequence in the host cell genome.

In related embodiments, the genome editing system is characterized in enhanced efficiency and precision of site-directed integration. Preferably, the efficiency and precision of site-directed integration enabled by genome editing system is enhanced by staggered overhangs on the donor nucleic acid sequence. In certain embodiments, the targeted polynucleotide sequence is double-stranded and contains a 5′ overhang wherein the overhang preferably comprises five nucleotides.

In various other embodiments, the polypeptide of the genome editing system comprises one or more mutations. More preferably, the mutation encodes a nuclease-deficient polypeptide. In various embodiments, the genome editing system comprises a fusion of one or more deaminases to the nuclease deficient polypeptide. Preferably, the one or more deaminases of the genome editing system is selected from adenine deaminase or cytosine deaminase. Use of cytidine deaminase and adenosine deaminase base editing is disclosed in U.S. Pat. No. 9,840,699. One approach is to produce an Cas12a fusion protein, preferably an inactive or nickase variant) and a base-editing enzyme or the active domain of a base editing enzyme. Cytidine deaminase and adenosine deaminase base editing is disclosed in U.S. Pat. No. 9,840,699. In various embodiments, the compositions comprise contacting a targeted polynucleotide sequence with a fusion protein comprising an Cas12a and one or more base-editing polypeptide such as a deaminase; and a gRNA targeting the fusion protein to the targeted polynucleotide sequence of the DNA strand. Accordingly, the fusion of one or more deaminases to the nuclease deficient polypeptide of the Cas12a genome editing system of enables base editing on DNA and/or RNA. In select embodiments, the system modifies one or more nucleobase on DNA and RNA. In related embodiments, the system enables multiplexed gene editing. Preferably, the genome editing system comprises a single crRNA. More preferably, the system enables targeting multiple genes simultaneously.

In yet other embodiments, the programmable polypeptide is operably linked to a nuclear localization signal (NLS). Preferably, the programmable polypeptide comprises an NLS on the N-terminus or the C-terminus or both or multiple NLS on the Cas12a polypeptide. In some embodiments, the polypeptide linked to the NLS further comprises crRNA to form a ribonucleoprotein complex. In some embodiments, polypeptide comprises one or more NLS repeats at either N- or C-terminus of the polypeptide.

In select embodiments, the one or more polypeptide sequences of the genome editing system comprises a modification, wherein the modification comprises a nuclease-deficient polypeptide (dCas). In related embodiments, the guide RNA of the genome editing system of comprises a prime editing guide RNA (pegRNA). Preferably, the pegRNA of the genome editing system hybridizes to a targeted polynucleotide sequence and acts as a primer to the one or more reverse transcriptases. More preferably, the pegRNA of the genome editing system binds to the nicked strand for initiation of repair through a reverse transcriptase using the repair template.

In various additional embodiments, the nuclease-deficient polypeptide of the genome editing system comprises a nickase activity. Preferably, the genome editing system comprises fusion of one or more reverse transcriptases to the nuclease deficient Cas (dCas). In certain examples, the fusion of one or more reverse transcriptases is selected from

### Optional Components/Modifications

### Donor Templates

In one embodiment, the compositions and systems herein may further comprise one or more donor templates for use in editing. In some cases, the donor template may comprise one or more polynucleotides. In certain cases, the donor template may comprise coding sequences for one or more polynucleotides. The donor template may be a DNA template. It may be single stranded or double stranded. It may also be circular single or double stranded. It may also be linear single stranded or double stranded. Without being bound by theory, the donor template may become integrated into the genome after a targeted cut by a programmable nuclease cutter described herein through cellular repair machinery including HDR and NHEJ. In various embodiments, the HDR donor is formed from reverse transcription of the ncRNA and forms the RT-DNA portion thereof.

The donor template, which may be integrated into a ncRNA and expressed as an RT-DNA product, may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor template alters a stop codon in the target polynucleotide. For example, the donor template may correct a premature stop codon. The correction may be achieved by deleting the stop codon or introduces one or more mutations to the stop codon. In other example embodiments, the donor template addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g. sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor templates that encode gene or gene fragments that compensate for or override defective gene expression such that cell phenotypes associated with defective gene expression are eliminated or changed to a different or desired cellular phenotype.

In an embodiment of the invention, the donor template may include, but not be limited to, genes or gene fragments, encoding proteins or RNA transcripts to be expressed, regulatory elements, repair templates, and the like. According to the invention, the donor templates may comprise left end and right end sequence elements that function with transposition components that mediate insertion.

In certain cases, the donor template manipulates a splicing site on the target polynucleotide. In some examples, the donor template disrupts a splicing site. The disruption may be achieved by inserting the polynucleotide to a splicing site and/or introducing one or more mutations to the splicing site. In certain examples, the donor template may restore a splicing site. For example, the polynucleotide may comprise a splicing site sequence.

The donor template to be inserted may has a size from 10 base pair or nucleotides to 50 kb in length, e.g., from 50 to 40k, from 100 and 30 k, from 100 to 10000, from 100 to 300, from 200 to 400, from 300 to 500, from 400 to 600, from 500 to 700, from 600 to 800, from 700 to 900, from 800 to 1000, from 900 to from 1100, from 1000 to 1200, from 1100 to 1300, from 1200 to 1400, from 1300 to 1500, from 1400 to 1600, from 1500 to 1700, from 600 to 1800, from 1700 to 1900, from 1800 to 2000 base pairs (bp) or nucleotides in length.

In some embodiments, the heterologous nucleic acid sequence is a donor DNA template that can be integrated into a host genome via HDR. In other embodiments, the heterologous nucleic acid sequence is a donor DNA template that can be integrated into a host genome via NHEJ.

In certain embodiments, the heterologous nucleic acid comprises or encodes a donor/template sequence, wherein the donor/template corrects/repairs/removes a mutation at the target genome site. For example, the mutation may be a mutated exon in a disease gene.

In certain embodiments, the donor/template may encode or comprises a functional DNA element, such as a promoter, an enhancer, a protein binding sequence, a methylation site, or a homology region for assisting gene editing, etc.

By “donor DNA” or “donor DNA template” it is meant a DNA segment (can be single stranded or double stranded DNA) to be inserted at a site cleaved by a gene-editing nuclease (e.g., a Cas9 or Cas12a nuclease) (e.g., after dsDNA cleavage, after nicking a target DNA, after dual nicking a target DNA, and the like). The donor DNA template can contain sufficient homology to a genomic sequence at the target site, e.g., 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. In the case of repair by NHEJ, no homology is needed on the donor DNA template against the site to which it targets editing. The donor template can be integrated into a ncRNA of a retron and expressed as a RT product as an RT-DNA.

Approximately 25, 50, 100, or 200 nucleotides, or more than 200 nucleotides, of sequence homology between a donor DNA template and a genomic sequence (or any integral value between 10 and 200 nucleotides, or more) can support homology-directed repair. Donor DNA template can be of any length, e.g., 50 nucleotides or more, 100 nucleotides or more, 250 nucleotides or more, 500 nucleotides or more, 1000 nucleotides or more, 5000 nucleotides or more, etc. A suitable donor DNA template can be from 50 nucleotides to 100 nucleotides, from 100 nucleotides to 500 nucleotides, from 500 nucleotides to 1000 nucleotides, from 1000 nucleotides to 5000 nucleotides, or from 5000 nucleotides to 10,000 nucleotides, or more than 10,000 nucleotides, in length.

As noted above, in some embodiments, the donor DNA template comprises a first homology arm and a second homology arm. The first homology arm is at or near the 5′ end of the donor DNA; and comprises a nucleotide sequence that is at least partially complementary to a first nucleotide sequence in a target nucleic acid. The second homology arm is at or near the 3′ end of the donor DNA; and comprises a nucleotide sequence that is at least partially complementary to a second nucleotide sequence in the target nucleic acid. The first and second homology arms can each independently have a length of from about 10 nucleotides to 400 nucleotides; e.g., from 10 nucleotides (nt) to 15 nt, from 15 nt to 20 nt, from 20 nt to 25 nt, from 25 nt to 30 nt, from 30 nt to 35 nt, from 35 nt to 40 nt, from 40 nt to 45 nt, from 45 nt to 50 nt, from 50 nt to 75 nt, from 75 nt to 100 nt, from 100 nt to 125 nt, from 125 nt to 150 nt, from 150 nt to 175 nt, from 175 nt to 200 nt, from 200 nt to 225 nt, from 225 nt to 250 nt, from 250 nt to 275 nt, from 275 nt to 300 nt, from 325 nt to 350 nt, from 350 nt to 375 nt, or from 375 nt to 400 nt.

In certain embodiments, the donor DNA template is used for editing the target nucleotide sequence. In certain embodiments, the donor DNA template comprises one or more mutations to be introduced into the target polynucleotide. Examples of such mutations include substitutions, deletions, insertions, or a combination thereof. In certain embodiments, the mutation causes a shift in an open reading frame on the target polynucleotide. In certain embodiments, the donor polynucleotide alters a stop codon in the target polynucleotide. In certain embodiments, the donor polynucleotide corrects a premature stop codon. The correction can be achieved by deleting the stop codon, or by introducing one or more sequence changes to alter the stop codon to a codon. In certain embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment includes a fragment less than the entire copy of a gene but otherwise provides sufficient nucleotide sequence to restore the functionality of a wild type gene or non-coding regulatory sequence (e.g., sequences encoding long non-coding RNA).

In certain embodiments, the donor DNA template may be used to replace a single allele of a defective gene or defective fragment thereof. In another embodiment, the donor DNA template is used to replace both alleles of a defective gene or defective gene fragment. A “defective gene” or “defective gene fragment” is a gene or portion of a gene that when expressed, fails to generate a functioning protein or non-coding RNA with functionality of the corresponding wild-type gene.

In certain example embodiments, these defective genes may be associated with one or more disease phenotypes. In certain example embodiments, the defective gene or gene fragment is not replaced but the heterologous nucleic acid is used to insert donor polynucleotides that encode gene or gene fragments that compensate for or override defective gene expression such that cell phenotypes associated with defective gene expression are eliminated or changed to a different or desired cellular phenotype. This can be achieved by including the coding sequence of a therapeutic protein, such as a therapeutic antibody or functional fragment thereof, or a wild-type version of a defective protein associated with one or more disease phenotypes.

In certain embodiments, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain embodiments, the donor DNA template manipulates a splicing site on the target polynucleotide. In certain embodiments, the donor DNA template disrupts a splicing site. The disruption may be achieved by inserting the polynucleotide to a splicing site and/or introducing one or more mutations to the splicing site. In certain embodiments, the donor polynucleotide may restore a splicing site. For example, the polynucleotide may comprise a splicing site sequence.

In certain embodiments, the donor DNA template to be inserted has a size from 10 bp to 50 kb in length, e.g., from 50 bp to ˜40 kb, from 100 bp to ˜30 kb, from 100 bp to ˜10 kb, from 100 bp to 300 bp, from 200 bp to 400 bp, from 300 bp to 500 bp, from 400 bp to 600 bp, from 500 bp to 700 bp, from 600 bp to 800 bp, from 700 bp to 900 bp, from 800 bp to 1000 bp, from 900 bp to 1100 bp, from 1000 bp to 1200 bp, from 1100 bp to 1300 bp, from 1200 bp to 1400 bp, from 1300 bp to 1500 bp, from 1400 bp to 1600 bp, from 1500 bp to 1700 bp, from 1600 bp to 1800 bp, from 1700 bp to 1900 bp, from 1800 bp to 2000 bp nucleotides in length.

In certain embodiments, the homologous arm on one or both ends of the sequence to be inserted is independently about 20 bp, 40 bp, 60 bp, 80 bp, 100 bp, 120 bp, or 150 bp.

The first homology arm and the second homology arm of the donor DNA flank a nucleotide sequence (“a nucleotide sequence of interest” or “an intervening nucleotide sequence”) that is to be introduced into a target nucleic acid. The nucleotide sequence of interest can comprise: i) a nucleotide sequence encoding a polypeptide of interest; ii) a nucleotide sequence encoding an exon of a gene; iii) a promoter sequence; iv) an enhancer sequence; v) a nucleotide sequence encoding a non-coding RNA; or vi) any combination of the foregoing.

The donor DNA can provide for gene correction, gene replacement, gene tagging, transgene insertion, nucleotide deletion, gene disruption, gene mutation, etc. For example, the donor DNA can be used to add, e.g., insert or replace, nucleic acid material to a target DNA (e.g. to “knock in” a nucleic acid that encodes a protein, an siRNA, an miRNA, etc.), to add a tag (e.g., 6× His, a fluorescent protein (e.g., a green fluorescent protein; a yellow fluorescent protein, etc.), hemagglutinin (HA), FLAG, etc.), to add a regulatory sequence to a gene (e.g. promoter, polyadenylation signal, internal ribosome entry sequence (IRES), 2A peptide, start codon, stop codon, splice signal, localization signal, enhancer, etc.), to modify a nucleic acid sequence (e.g., introduce a mutation), and the like. For example, the donor DNA can be used to modify DNA in a site-specific, i.e. “targeted”, way; for example gene knock-out, gene knock-in, gene editing, gene tagging, etc., as used in, for example, gene therapy, e.g. to treat a disease; or as an antiviral, antipathogenic, or anticancer therapeutic, the production of genetically modified organisms in agriculture, the large scale production of proteins by cells for therapeutic, diagnostic, or research purposes, the induction of pluripotent stem cells, biological research, the targeting of genes of pathogens for deletion or replacement, etc.

In some cases, the donor DNA comprises a nucleotide sequence encoding a polypeptide of interest. Polypeptides of interest include, e.g., a) functional versions of a polypeptide that comprises one or more amino acid substitutions, insertions, and/or deletions and that exhibits reduced function, e.g., where the reduced function is associated with or causes a pathological condition; b) fluorescent polypeptides; c) hormones; d) receptors for ligands; e) ion channels; f) neurotransmitters; g) and the like.

In some cases, the donor DNA comprises a nucleotide sequence that encodes a wild-type protein that is lacking in the recipient cell. In some cases, the donor DNA encodes a wild type factor (e.g. Factor VII, Factor VIII, Factor IX and the like) involved in coagulation. In some cases, the donor DNA comprises a nucleotide sequence that encodes a therapeutic antibody. In some cases, the donor DNA comprises a nucleotide sequence that encodes an engineered protein or receptor. In some cases, the engineered receptor is a T cell receptor (TCR), a natural killer (NK) receptor (NKR), or a B cell receptor (BCR). In some cases, the engineered TCR or NKR targets a cancer marker (e.g., a polypeptide that is expressed (e.g., over-expressed) on the surface of a cancer cell). In some cases, the donor DNA comprises a nucleotide sequence that encodes a chimeric antigen receptor (CAR). In some cases, the CAR targets a cancer marker. Donor DNAs encoding CAR, TCR, and/or NCR proteins may be folded into DNA origami structures (DNA nanostructures) and delivered into T cells or NK cells in vitro or in vivo.

Non-limiting examples of polypeptides that can be encoded by a donor DNA include, e.g., IL1B (interleukin 1, beta), XDH (xanthine dehydrogenase), TP53 (tumor protein p53), PTGIS (prostaglandin 12 (prostacyclin) synthase), MB (myoglobin), IL4 (interleukin 4), ANGPT1 (angiopoietin 1), ABCG8 (ATP-binding cassette, sub-family G (WHITE), member 8), CTSK (cathepsin K), PTGIR (prostaglandin 12 (prostacyclin) receptor (IP)), KCNJ11 (potassium inwardly-rectifying channel, subfamily J, member 11), INS (insulin), CRP (C-reactive protein, pentraxin-related), PDGFRB (platelet-derived growth factor receptor, beta polypeptide), CCNA2 (cyclin A2), PDGFB (platelet-derived growth factor beta polypeptide (simian sarcoma viral (v-sis) oncogene homolog)), KCNJ5 (potassium inwardly-rectifying channel, subfamily J, member 5), KCNN3 (potassium intermediate/small conductance calcium-activated channel, subfamily N, member 3), CAPN10 (calpain 10), PTGES (prostaglandin E synthase), ADRA2B (adrenergic, alpha-2B-, receptor), ABCG5 (ATP-binding cassette, sub-family G (WHITE), member 5), PRDX2 (peroxiredoxin 2), CAPN5 (calpain 5), PARP14 (poly (ADP-ribose) polymerase family, member 14), MEX3C (mex-3 homolog C (C. elegans)), ACE angiotensin I converting enzyme (peptidyl-dipeptidase A) 1), TNF (tumor necrosis factor (TNF superfamily, member 2)), IL6 (interleukin 6 (interferon, beta 2)), STN (statin), SERPINE1 (serpin peptidase inhibitor, clade E (nexin, plasminogen activator inhibitor type 1), member 1), ALB (albumin), ADIPOQ (adiponectin, C1Q and collagen domain containing), APOB (apolipoprotein B (including Ag(x) antigen)), APOE (apolipoprotein E), LEP (leptin), MTHFR (5,10-methylenetetrahydrofolate reductase (NADPH)), APOA1 (apolipoprotein A-I), EDN1 (endothelin 1), NPPB (natriuretic peptide precursor B), NOS3 (nitric oxide synthase 3 (endothelial cell)), PPARG (peroxisome proliferator-activated receptor gamma), PLAT (plasminogen activator, tissue), PTGS2 (prostaglandin-endoperoxide synthase 2 (prostaglandin G/H synthase and cyclooxygenase)), CETP (cholesteryl ester transfer protein, plasma), AGTR1 (angiotensin II receptor, type 1), HMGCR (3-hydroxy-3-methylglutaryl-Coenzyme A reductase), IGF1 (insulin-like growth factor 1 (somatomedin C)), SELE (selectin E), REN (renin), PPARA (peroxisome proliferator-activated receptor alpha), PON1 (paraoxonase 1), KNG1 (kininogen 1), CCL2 (chemokine (C-C motif) ligand 2), LPL (lipoprotein lipase), vWF (von Willebrand factor), F2 (coagulation factor II (thrombin)), ICAM1 (intercellular adhesion molecule 1), TGFB1 (transforming growth factor, beta 1), NPPA (natriuretic peptide precursor A), IL10 (interleukin 10), EPO (erythropoietin), SOD1 (superoxide dismutase 1, soluble), VCAM1 (vascular cell adhesion molecule 1), IFNG (interferon, gamma), LPA (lipoprotein, Lp(a)), MPO (myeloperoxidase), ESR1 (estrogen receptor 1), MAPK1 (mitogen-activated protein kinase 1), HP (haptoglobin), F3 (coagulation factor III (thromboplastin, tissue factor)), CST3 (cystatin C), COG2 (component of oligomeric Golgi complex 2), MMP9 (matrix metallopeptidase 9 (gelatinase B, 92 kDa gelatinase, 92 kDa type IV collagenase)), SERPINC1 (serpin peptidase inhibitor, clade C (antithrombin), member 1), F8 (coagulation factor VIII, procoagulant component), HMOX1 (heme oxygenase (decycling) 1), APOC3 (apolipoprotein C-III), IL8 (interleukin 8), PROK1 (prokineticin 1), CBS (cystathionine-beta-synthase), NOS2 (nitric oxide synthase 2, inducible), TLR4 (toll-like receptor 4), SELP (selectin P (granule membrane protein 140 kDa, antigen CD62)), ABCA1 (ATP-binding cassette, sub-family A (ABC1), member 1), AGT (angiotensinogen (serpin peptidase inhibitor, clade A, member 8)), LDLR (low density lipoprotein receptor), GPT (glutamic-pyruvate transaminase (alanine aminotransferase)), VEGFA (vascular endothelial growth factor A), NR3C2 (nuclear receptor subfamily 3, group C, member 2), IL18 (interleukin 18 (interferon-gamma-inducing factor)), NOS1 (nitric oxide synthase 1 (neuronal)), NR3C1 (nuclear receptor subfamily 3, group C, member 1 (glucocorticoid receptor)), FGB (fibrinogen beta chain), HGF (hepatocyte growth factor (hepapoietin A; scatter factor)), ILIA (interleukin 1, alpha), RETN (resistin), AKT1 (v-akt murine thymoma viral oncogene homolog 1), LIPC (lipase, hepatic), HSPD1 (heat shock 60 kDa protein 1 (chaperonin)), MAPK14 (mitogen-activated protein kinase 14), SPP1 (secreted phosphoprotein 1), ITGB3 (integrin, beta 3 (platelet glycoprotein 111a, antigen CD61)), CAT (catalase), UTS2 (urotensin 2), THBD (thrombomodulin), F10 (coagulation factor X), CP (ceruloplasmin (ferroxidase)), TNFRSF11B (tumor necrosis factor receptor superfamily, member lib), EDNRA (endothelin receptor type A), EGFR (epidermal growth factor receptor (erythroblastic leukemia viral (v-erb-b) oncogene homolog, avian)), MMP2 (matrix metallopeptidase 2 (gelatinase A, 72 kDa gelatinase, 72 kDa type IV collagenase)), PLG (plasminogen), NPY (neuropeptide Y), RHOD (ras homolog gene family, member D), MAPK8 (mitogen-activated protein kinase 8), MYC (v-myc myelocytomatosis viral oncogene homolog (avian)), FN1 (fibronectin 1), CMA1 (chymase 1, mast cell), PLAU (plasminogen activator, urokinase), GNB3 (guanine nucleotide binding protein (G protein), beta polypeptide 3), ADRB2 (adrenergic, beta-2-, receptor, surface), APOA5 (apolipoprotein A-V), SOD2 (superoxide dismutase 2, mitochondrial), F5 (coagulation factor V (proaccelerin, labile factor)), VDR (vitamin D (1,25-dihydroxyvitamin D3) receptor), ALOX5 (arachidonate 5-lipoxygenase), HLA-DRB1 (major histocompatibility complex, class II, DR beta 1), PARP1 (poly (ADP-ribose) polymerase 1), CD40LG (CD40 ligand), PON2 (paraoxonase 2), AGER (advanced glycosylation end product-specific receptor), IRS1 (insulin receptor substrate 1), PTGS1 (prostaglandin-endoperoxide synthase 1 (prostaglandin G/H synthase and cyclooxygenase)), ECE1 (endothelin converting enzyme 1), F7 (coagulation factor VII (serum prothrombin conversion accelerator)), URN (interleukin 1 receptor antagonist), EPHX2 (epoxide hydrolase 2, cytoplasmic), IGFBP1 (insulin-like growth factor binding protein 1), MAPK10 (mitogen-activated protein kinase 10), FAS (Fas (TNF receptor superfamily, member 6)), ABCB1 (ATP-binding cassette, sub-family B (MDR/TAP), member 1), JUN (jun oncogene), IGFBP3 (insulin-like growth factor binding protein 3), CD14 (CD14 molecule), PDE5A (phosphodiesterase 5A, cGMP-specific), AGTR2 (angiotensin II receptor, type 2), CD40 (CD40 molecule, TNF receptor superfamily member 5), LCAT (lecithin-cholesterol acyltransferase), CCR5 (chemokine (C-C motif) receptor 5), MMP1 (matrix metallopeptidase 1 (interstitial collagenase)), TIMP1 (TIMP metallopeptidase inhibitor 1), ADM (adrenomedullin), DYT10 (dystonia 10), STAT3 (signal transducer and activator of transcription 3 (acute-phase response factor)), MMP3 (matrix metallopeptidase 3 (stromelysin 1, progelatinase)), ELN (elastin), USF1 (upstream transcription factor 1), CFH (complement factor H), HSPA4 (heat shock 70 kDa protein 4), MMP12 (matrix metallopeptidase 12 (macrophage elastase)), MME (membrane metallo-endopeptidase), F2R (coagulation factor II (thrombin) receptor), SELL (selectin L), CTSB (cathepsin B), ANXA5 (annexin A5), ADRB1 (adrenergic, beta-1-, receptor), CYBA (cytochrome b-245, alpha polypeptide), FGA (fibrinogen alpha chain), GGT1 (gamma-glutamyltransferase 1), LIPG (lipase, endothelial), HIF1A (hypoxia inducible factor 1, alpha subunit (basic helix-loop-helix transcription factor)), CXCR4 (chemokine (C-X-C motif) receptor 4), PROC (protein C (inactivator of coagulation factors Va and Villa)), SCARB1 (scavenger receptor class B, member 1), CD79A (CD79a molecule, immunoglobulin-associated alpha), PLTP (phospholipid transfer protein), ADD1 (adducin 1 (alpha)), FGG (fibrinogen gamma chain), SAA1 (serum amyloid Al), KCNH2 (potassium voltage-gated channel, subfamily H (eag-related), member 2), DPP4 (dipeptidyl-peptidase 4), G6PD (glucose-6-phosphate dehydrogenase), NPR1 (natriuretic peptide receptor A/guanylate cyclase A (atrionatriuretic peptide receptor A)), VTN (vitronectin), KIAA0101 (KIAA0101), FOS (FBJ murine osteosarcoma viral oncogene homolog), TLR2 (toll-like receptor 2), PPIG (peptidylprolyl isomer ase G (cyclophilin G)), IL1R1 (interleukin 1 receptor, type I), AR (androgen receptor), CYP1A1 (cytochrome P450, family 1, subfamily A, polypeptide 1), SERPINA1 (serpin peptidase inhibitor, clade A (alpha-1 antiproteinase, antitrypsin), member 1), MTR (5-methyltetrahydrofolate-homocysteine methyltransferase), RBP4 (retinol binding protein 4, plasma), APOA4 (apolipoprotein A-IV), CDKN2A (cyclin-dependent kinase inhibitor 2A (melanoma, p16, inhibits CDK4)), FGF2 (fibroblast growth factor 2 (basic)), EDNRB (endothelin receptor type B), ITGA2 (integrin, alpha 2 (CD49B, alpha 2 subunit of VLA-2 receptor)), CAB INI (calcineurin binding protein 1), SHBG (sex hormone-binding globulin), HMGB1 (high-mobility group box 1), HSP90B2P (heat shock protein 90 kDa beta (Grp94), member 2 (pseudogene)), CYP3A4 (cytochrome P450, family 3, subfamily A, polypeptide 4), GJA1 (gap junction protein, alpha 1, 43 kDa), CAV1 (caveolin 1, caveolae protein, 22 kDa), ESR2 (estrogen receptor 2 (ER beta)), LTA (lymphotoxin alpha (TNF superfamily, member 1)), GDF15 (growth differentiation factor 15), BDNF (brain-derived neurotrophic factor), CYP2D6 (cytochrome P450, family 2, subfamily D, polypeptide 6), NGF (nerve growth factor (beta polypeptide)), SP1 (Sp 1 transcription factor), TGIF1 (TGFB-induced factor homeobox 1), SRC (v-src sarcoma (Schmidt-Ruppin A-2) viral oncogene homolog (avian)), EGF (epidermal growth factor (beta-urogastrone)), PIK3CG (phosphoinositide-3-kinase, catalytic, gamma polypeptide), HLA-A (major histocompatibility complex, class I, A), KCNQ1 (potassium voltage-gated channel, KQT-like subfamily, member 1), CNR1 (cannabinoid receptor 1 (brain)), FBN1 (fibrillin 1), CHKA (choline kinase alpha), BEST1 (bestrophin 1), APP (amyloid beta (A4) precursor protein), CTNNB1 (catenin (cadherin-associated protein), beta 1, 88 kDa), IL2 (interleukin 2), CD36 (CD36 molecule (thrombospondin receptor)), PRKAB1 (protein kinase, AMP-activated, beta 1 non-catalytic subunit), TPO (thyroid peroxidase), ALDH7A1 (aldehyde dehydrogenase 7 family, member Al), CX3CR1 (chemokine (C-X3-C motif) receptor 1), TH (tyrosine hydroxylase), F9 (coagulation factor IX), GH1 (growth hormone 1), TF (transferrin), HFE (hemochromatosis), IE17A (interleukin 17A), PTEN (phosphatase and tensin homolog), GSTM1 (glutathione S-transferase mu 1), DMD (dystrophin), GATA4 (GATA binding protein 4), F13A1 (coagulation factor XIII, Al polypeptide), TTR (transthyretin), FABP4 (fatty acid binding protein 4, adipocyte), PON3 (paraoxonase 3), APOC1 (apolipoprotein C-I), INSR (insulin receptor), TNFRSF1B (tumor necrosis factor receptor superfamily, member IB), HTR2A (5-hydroxytryptamine (serotonin) receptor 2A), CSF3 (colony stimulating factor 3 (granulocyte)), CYP2C9 (cytochrome P450, family 2, subfamily C, polypeptide 9), TXN (thioredoxin), CYP11B2 (cytochrome P450, family 11, subfamily B, polypeptide 2), PTH (parathyroid hormone), CSF2 (colony stimulating factor 2 (granulocyte-macrophage)), KDR (kinase insert domain receptor (a type III receptor tyrosine kinase)), PLA2G2A (phospholipase A2, group IIA (platelets, synovial fluid)), B2M (beta-2-microglobulin), THBS1 (thrombospondin 1), GCG (glucagon), RHOA (ras homolog gene family, member A), ALDH2 (aldehyde dehydrogenase 2 family (mitochondrial)), TCF7L2 (transcription factor 7-like 2 (T-cell specific, HMG-box)), BDKRB2 (bradykinin receptor B2), NFE2L2 (nuclear factor (erythroid-derived 2)-like 2), NOTCH1 (Notch homolog 1, translocation-associated (Drosophila)), UGT1A1 (UDP glucuronosyltransferase 1 family, polypeptide Al), IFNA1 (interferon, alpha 1), PPARD (peroxisome proliferator-activated receptor delta), SIRT1 (sirtuin (silent mating type information regulation 2 homolog) 1 (S. cerevisiae)), GNRH1 (gonadotropin-releasing hormone 1 (luteinizing-releasing hormone)), PAPPA (pregnancy-associated plasma protein A, pappalysin 1), ARR3 (arrestin 3, retinal (X-arrestin)), NPPC (natriuretic peptide precursor C), AHSP (alpha hemoglobin stabilizing protein), PTK2 (PTK2 protein tyrosine kinase 2), IL13 (interleukin 13), MTOR (mechanistic target of rapamycin (serine/threonine kinase)), ITGB2 (integrin, beta 2 (complement component 3 receptor 3 and 4 subunit)), GSTT1 (glutathione S-transferase theta 1), IL6ST (interleukin 6 signal transducer (gpl30, oncostatin M receptor)), CPB2 (carboxypeptidase B2 (plasma)), CYP1A2 (cytochrome P450, family 1, subfamily A, polypeptide 2), HNF4A (hepatocyte nuclear factor 4, alpha), SLC6A4 (solute carrier family 6 (neurotransmitter transporter, serotonin), member 4), PLA2G6 (phospholipase A2, group VI (cytosolic, calcium-independent)), TNFSF11 (tumor necrosis factor (ligand) superfamily, member 11), SLC8A1 (solute carrier family 8 (sodium/calcium exchanger), member 1), F2RL1 (coagulation factor II (thrombin) receptor-like 1), AKR1A1 (aldo-keto reductase family 1, member A1 (aldehyde reductase)), ALDH9A1 (aldehyde dehydrogenase 9 family, member Al), BGLAP (bone gamma-carboxyglutamate (gla) protein), MTTP (microsomal triglyceride transfer protein), MTRR (5-methyltetrahydrofolate-homocysteine methyltransferase reductase), SULT1A3 (sulfotransferase family, cytosolic, 1A, phenol-preferring, member 3), RAGE (renal tumor antigen), C4B (complement component 4B (Chido blood group), P2RY12 (purinergic receptor P2Y, G-protein coupled, 12), RNLS (renalase, FAD-dependent amine oxidase), CREBI (cAMP responsive element binding protein 1), POMC (proopiomelanocortin), RAC1 (ras-related C3 botulinum toxin substrate 1 (rho family, small GTP binding protein Racl)), LMNA (lamin NC), CD59 (CD59 molecule, complement regulatory protein), SCN5A (sodium channel, voltage-gated, type V, alpha subunit), CYP1B1 (cytochrome P450, family 1, subfamily B, polypeptide 1), MIF (macrophage migration inhibitory factor (glycosylation-inhibiting factor)), MMP13 (matrix metallopeptidase 13 (collagenase 3)), TIM P2 (TIMP metallopeptidase inhibitor 2), CYP19A1 (cytochrome P450, family 19, subfamily A, polypeptide 1), CYP21A2 (cytochrome P450, family 21, subfamily A, polypeptide 2), PTPN22 (protein tyrosine phosphatase, non-receptor type 22 (lymphoid)), MYH14 (myosin, heavy chain 14, non-muscle), MBL2 (mannose-binding lectin (protein C) 2, soluble (opsonic defect)), SELPLG (selectin P ligand), AOC3 (amine oxidase, copper containing 3 (vascular adhesion protein 1)), CTSL1 (cathepsin LI), PCNA (proliferating cell nuclear antigen), IGF2 (insulin like growth factor 2 (somatomedin A)), ITGB1 (integrin, beta 1 (fibronectin receptor, beta polypeptide, antigen CD29 includes MDF2, MSK12)), CAST (calpastatin), CXCL12 (chemokine (C-X-C motif) ligand 12 (stromal cell-derived factor 1)), IGHE (immunoglobulin heavy constant epsilon), KCNE1 (potassium voltage-gated channel, Isk-related family, member 1), TFRC (transferrin receptor (p90, CD71)), COL1A1 (collagen, type I, alpha 1), COL1A2 (collagen, type I, alpha 2), IL2RB (interleukin 2 receptor, beta), PLA2G10 (phospholipase A2, group X), ANGPT2 (angiopoietin 2), PROCR (protein C receptor, endothelial (EPCR)), NOX4 (NADPH oxidase 4), HAMP (hepcidin antimicrobial peptide), PTPN11 (protein tyrosine phosphatase, non-receptor type 11), SLC2A1 (solute carrier family 2 (facilitated glucose transporter), member 1), IL2RA (interleukin 2 receptor, alpha), CCL5 (chemokine (C-C motif) ligand 5), IRF1 (interferon regulatory factor 1), CFLAR (CASP8 and FADD-like apoptosis regulator), CALC A (calcitonin-related polypeptide alpha), EIF4E (eukaryotic translation initiation factor 4E), GSTP1 (glutathione S-transferase pi 1), JAK2 (Janus kinase 2), CYP3A5 (cytochrome P450, family 3, subfamily A, polypeptide 5), HSPG2 (heparan sulfate proteoglycan 2), CCL3 (chemokine (C-C motif) ligand 3), MYD88 (myeloid differentiation primary response gene (88)), VIP (vasoactive intestinal peptide), SOAT1 (sterol O-acyltransferase 1), ADRBK1 (adrenergic, beta, receptor kinase 1), NR4A2 (nuclear receptor subfamily 4, group A, member 2), MMP8 (matrix metallopeptidase 8 (neutrophil collagenase)), NPR2 (natriuretic peptide receptor B/guanylate cyclase B (atrionatriuretic peptide receptor B)), GCH1 (GTP cyclohydrolase 1), EPRS (glutamyl-prolyl-tRNA synthetase), PPARGC1A (peroxisome proliferator-activated receptor gamma, coactivator 1 alpha), F12 (coagulation factor XII (Hageman factor)), PEC AMI (platelet/endothelial cell adhesion molecule), CCL4 (chemokine (C-C motif) ligand 4), SERPINA3 (serpin peptidase inhibitor, clade A (alpha-1 antiproteinase, antitrypsin), member 3), CASR (calcium-sensing receptor), GJA5 (gap junction protein, alpha 5, 40 kDa), FABP2 (fatty acid binding protein 2, intestinal), TTF2 (transcription termination factor, RNA polymerase II), PROS1 (protein S (alpha)), CTF1 (cardiotrophin 1), SGCB (sarcoglycan, beta (43 kDa dystrophin-associated glycoprotein)), YME1L1 (YMEl-like 1 (S. cerevisiae)), CAMP (cathelicidin antimicrobial peptide), ZC3H12A (zinc finger CCCH-type containing 12A), AKR1B1 (aldo-keto reductase family 1, member B1 (aldose reductase)), DES (desmin), MMP7 (matrix metallopeptidase 7 (matrilysin, uterine)), AHR (aryl hydrocarbon receptor), CSF1 (colony stimulating factor 1 (macrophage)), HDAC9 (histone deacetylase 9), CTGF (connective tissue growth factor), KCNMA1 (potassium large conductance calcium-activated channel, subfamily M, alpha member 1), UGT1A (UDP glucuronosyltransferase 1 family, polypeptide A complex locus), PRKCA (protein kinase C, alpha), COMT (catechol-b-methyltransf erase), S100B (S100 calcium binding protein B), EGR1 (early growth response 1), PRL (prolactin), IL15 (interleukin 15), DRD4 (dopamine receptor D4), CAMK2G (calcium/calmodulin-dependent protein kinase II gamma), SLC22A2 (solute carrier family 22 (organic cation transporter), member 2), CCL11 (chemokine (C-C motif) ligand 11), PGF (placental growth factor), THPO (thrombopoietin), GP6 (glycoprotein VI (platelet)), TACR1 (tachykinin receptor 1), NTS (neurotensin), HNF1A (HNF1 homeobox A), SST (somatostatin), KCND1 (potassium voltage-gated channel, Shal-related subfamily, member 1), LOC646627 (phospholipase inhibitor), TBXAS1 (thromboxane A synthase 1 (platelet)), CYP2J2 (cytochrome P450, family 2, subfamily J, polypeptide 2), TBXA2R (thromboxane A2 receptor), ADH1C (alcohol dehydrogenase 1C (class I), gamma polypeptide), ALOX12 (arachidonate 12-lipoxygenase), AHSG (alpha-2-HS-glycoprotein), BHMT (betaine-homocysteine methyltransferase), GJA4 (gap junction protein, alpha 4, 37 kDa), SLC25A4 (solute carrier family 25 (mitochondrial carrier; adenine nucleotide translocator), member 4), ACLY (ATP citrate lyase), ALOX5AP (arachidonate 5-lipoxygenase-activating protein), NUMA1 (nuclear mitotic apparatus protein 1), CYP27B1 (cytochrome P450, family 27, subfamily B, polypeptide 1), CYSLTR2 (cysteinyl leukotriene receptor 2), SOD3 (superoxide dismutase 3, extracellular), LTC4S (leukotriene C4 synthase), UCN (urocortin), GHRL (ghrelin/obestatin prepropeptide), APOC2 (apolipoprotein C-II), CLEC4A (C-type lectin domain family 4, member A), KBTBD10 (kelch repeat and BTB (POZ) domain containing 10), INC (tenascin C), TYMS (thymidylate synthetase), SHC1 (SHC (Src homology 2 domain containing) transforming protein 1), LRP1 (low density lipoprotein receptor-related protein 1), SOCS3 (suppressor of cytokine signaling 3), ADH1B (alcohol dehydrogenase IB (class I), beta polypeptide), KLK3 (kallikrein-related peptidase 3), HSD11B1 (hydroxysteroid (11-beta) dehydrogenase 1), VKORC1 (vitamin K epoxide reductase complex, subunit 1), SERPINB2 (serpin peptidase inhibitor, clade B (ovalbumin), member 2), TNS1 (tensin 1), RNF19A (ring finger protein 19A), EPOR (erythropoietin receptor), ITGAM (integrin, alpha M (complement component 3 receptor 3 subunit)), PITX2 (paired-like homeodomain 2), MAPK7 (mitogen-activated protein kinase 7), FCGR3A (Fc fragment of IgG, low affinity 111a, receptor (CD16a)), LEPR (leptin receptor), ENG (endoglin), GPX1 (glutathione peroxidase 1), GOT2 (glutamic-oxaloacetic transaminase 2, mitochondrial (aspartate aminotransferase 2)), HRH1 (histamine receptor HI), NR112 (nuclear receptor subfamily 1, group I, member 2), CRH (corticotropin releasing hormone), HTR1A (5-hydroxytryptamine (serotonin) receptor 1A), VDAC1 (voltage-dependent anion channel 1), HPSE (heparanase), SFTPD (surfactant protein D), TAP2 (transporter 2, ATP-binding cassette, sub-family B (MDR/TAP)), RNF123 (ring finger protein 123), PTK2B (PTK2B protein tyrosine kinase 2 beta), NTRK2 (neurotrophic tyrosine kinase, receptor, type 2), IL6R (interleukin 6 receptor), ACHE (acetylcholinesterase (Yt blood group)), GLP1R (glucagon-like peptide 1 receptor), GHR (growth hormone receptor), GSR (glutathione reductase), NQOl (NAD(P)H dehydrogenase, quinone 1), NR5A1 (nuclear receptor subfamily 5, group A, member 1), GJB2 (gap junction protein, beta 2, 26 kDa), SLC9A1 (solute carrier family 9 (sodium/hydrogen exchanger), member 1), MAOA (monoamine oxidase A), PCSK9 (proprotein convertase subtilisin/kexin type 9), FCGR2A (Fc fragment of IgG, low affinity Ila, receptor (CD32)), SERPINF1 (serpin peptidase inhibitor, clade F (alpha-2 antiplasmin, pigment epithelium derived factor), member 1), EDN3 (endothelin 3), DHFR (dihydrofolate reductase), GAS6 (growth arrest-specific 6), SMPD1 (sphingomyelin phosphodiesterase 1, acid lysosomal), UCP2 (uncoupling protein 2 (mitochondrial, proton carrier)), TFAP2A (transcription factor AP-2 alpha (activating enhancer binding protein 2 alpha)), C4BPA (complement component 4 binding protein, alpha), SERPINF2 (serpin peptidase inhibitor, clade F (alpha-2 antiplasmin, pigment epithelium derived factor), member 2), TYMP (thymidine phosphorylase), ALPP (alkaline phosphatase, placental (Regan isozyme)), CXCR2 (chemokine (C-X-C motif) receptor 2), SLC39A3 (solute carrier family 39 (zinc transporter), member 3), ABCG2 (ATP-binding cassette, sub-family G (WHITE), member 2), ADA (adenosine deaminase), JAK3 (Janus kinase 3), HSPA1A (heat shock 70 kDa protein 1A), FASN (fatty acid synthase), FGF1 (fibroblast growth factor 1 (acidic)), Fll (coagulation factor XI), ATP7A (ATPase, Cu++ transporting, alpha polypeptide), CR1 (complement component (3b/4b) receptor 1 (Knops blood group)), GFAP (glial fibrillary acidic protein), ROCK1 (Rho-associated, coiled-coil containing protein kinase 1), MECP2 (methyl CpG binding protein 2 (Rett syndrome)), MYLK (myosin light chain kinase), BCFlE (butyrylcholinesterase), LIPE (lipase, hormone-sensitive), PRDX5 (peroxiredoxin 5), ADORA1 (adenosine A1 receptor), WRN (Werner syndrome, RecQ helicase-like), CXCR3 (chemokine (C-X-C motif) receptor 3), CD81 (CD81 molecule), SMAD7 (SMAD family member 7), LAMC2 (laminin, gamma 2), MAP3K5 (mitogen-activated protein kinase kinase kinase 5), CF1GA (chromogranin A (parathyroid secretory protein 1)), IAPP (islet amyloid polypeptide), RFIO (rhodopsin), ENPP1 (ectonucleotide pyrophosphatase/phosphodiesterase 1), PTF1LF1 (parathyroid hormone-like hormone), NRG1 (neuregulin 1), VEGFC (vascular endothelial growth factor C), ENPEP (glutamyl aminopeptidase (aminopeptidase A)), CEBPB (CCAAT/enhancer binding protein (C/EBP), beta), NAGLU (N-acetylglucosaminidase, alpha), F2RL3 (coagulation factor II (thrombin) receptor-like 3), CX3CL1 (chemokine (C-X3-C motif) ligand 1), BDKRB1 (bradykinin receptor Bl), ADAMTS13 (ADAM metallopeptidase with thrombospondin type 1 motif, 13), ELANE (elastase, neutrophil expressed), ENPP2 (ectonucleotide pyrophosphatase/phosphodiesterase 2), CISFI (cytokine inducible SF12-containing protein), GAST (gastrin), MYOC (myocilin, trabecular mesh work inducible glucocorticoid response), ATP1A2 (ATPase, Na+/K+ transporting, alpha 2 polypeptide), NF1 (neurofibromin 1), GJB1 (gap junction protein, beta 1, 32 kDa), MEF2A (myocyte enhancer factor 2A), VCL (vinculin), BMPR2 (bone morphogenetic protein receptor, type II (serine/threonine kinase)), TUBB (tubulin, beta), CDC42 (cell division cycle 42 (GTP binding protein, 25 kDa)), KRT18 (keratin 18), F1SF1 (heat shock transcription factor 1), MYB (v-myb myeloblastosis viral oncogene homolog (avian)), PRKAA2 (protein kinase, AMP-activated, alpha 2 catalytic subunit), ROCK2 (Rho-associated, coiled-coil containing protein kinase 2), TFPI (tissue factor pathway inhibitor (lipoprotein-associated coagulation inhibitor)), PRKG1 (protein kinase, cGMP-dependent, type I), BMP2 (bone morphogenetic protein 2), CTNND1 (catenin (cadherin-associated protein), delta 1), CTF1 (cystathionase (cystathionine gamma-lyase)), CTSS (cathepsin S), VAV2 (vav 2 guanine nucleotide exchange factor), NPY2R (neuropeptide Y receptor Y2), IGFBP2 (insulin-like growth factor binding protein 2, 36 kDa), CD28 (CD28 molecule), GSTA1 (glutathione S-transferase alpha 1), PPIA (peptidylprolyl isomerase A (cyclophilin A)), APOF1 (apolipoprotein FI (beta-2-glycoprotein I)), S100A8 (S100 calcium binding protein A8), IL11 (interleukin 11), ALOX15 (arachidonate 15-lipoxygenase), FBLN1 (fibulin 1), NR1F13 (nuclear receptor subfamily 1, group FI, member 3), SCD (stearoyl-CoA desaturase (delta-9-desaturase)), GIP (gastric inhibitory polypeptide), CF1 GB (chromogranin B (secretogranin 1)), PRKCB (protein kinase C, beta), SRD5A1 (steroid-5-alpha-reductase, alpha polypeptide 1 (3-oxo-5 alpha-steroid delta 4-dehydrogenase alpha 1)), F1SD11B2 (hydroxy steroid (11-beta) dehydrogenase 2), CALCRL (calcitonin receptor-like), GALNT2 (UDP-N-acetyl-alpha-D-galactosamine.polypeptide N-acetylgalactosaminyltransferase 2 (GalNAc-T2)), ANGPTL4 (angiopoietin-like 4), KCNN4 (potassium intermediate/small conductance calcium-activated channel, subfamily N, member 4), PIK3C2A (phosphoinositide-3-kinase, class 2, alpha polypeptide), HBEGF (heparin-binding EGF-like growth factor), CYP7A1 (cytochrome P450, family 7, subfamily A, polypeptide 1), HLA-DRB5 (major histocompatibility complex, class II, DR beta 5), BNIP3 (BCL2/adeno virus E1B 19 kDa interacting protein 3), GCKR (glucokinase (hexokinase 4) regulator), S100A12 (S100 calcium binding protein A 12), PADI4 (peptidyl arginine deaminase, type IV), HSPA14 (heat shock 70 kDa protein 14), CXCR1 (chemokine (C-X-C motif) receptor 1), H19 (H19, imprinted maternally expressed transcript (non-protein coding)), KRTAP19-3 (keratin associated protein 19-3), insulin, RAC2 (ras-related C3 botulinum toxin substrate 2 (rho family, small GTP binding protein Rac2)), RYR1 (ryanodine receptor 1 (skeletal)), CLOCK (clock homolog (mouse)), NGFR (nerve growth factor receptor (TNFR superfamily, member 16)), DBH (dopamine beta-hydroxylase (dopamine beta-monooxygenase)), CHRNA4 (cholinergic receptor, nicotinic, alpha 4), CACNAlC (calcium channel, voltage-dependent, L type, alpha 1C subunit), PRKAG2 (protein kinase, AMP-activated, gamma 2 non-catalytic subunit), CHAT (choline acetyltransferase), PTGDS (prostaglandin D2 synthase 21 kDa (brain)), NR1H2 (nuclear receptor subfamily 1, group H, member 2), TEK (TEK tyrosine kinase, endothelial), VEGFB (vascular endothelial growth factor B), MEF2C (myocyte enhancer factor 2C), MAPKAPK2 (mitogen-activated protein kinase-activated protein kinase 2), TNFRSF11 A (tumor necrosis factor receptor superfamily, member 11a, NFKB activator), HSPA9 (heat shock 70 kDa protein 9 (mortalin)), CYSLTR1 (cysteinyl leukotriene receptor 1), MAT1A (methionine adenosyltransferase 1, alpha), OPRL1 (opiate receptor-like 1), IMPA1 (inositol(myo)-l(or 4)-monophosphatase 1), CLCN2 (chloride channel 2), DLD (dihydrolipoamide dehydrogenase), PSMA6 (proteasome (prosome, macropain) subunit, alpha type, 6), PSMB8 (proteasome (prosome, macropain) subunit, beta type, 8 (large multifunctional peptidase 7)), CHI3L1 (chitinase 3-like 1 (cartilage glycoprotein-39)), ALDH1B1 (aldehyde dehydrogenase 1 family, member Bl), PARP2 (poly (ADP-ribose) polymerase 2), STAR (steroidogenic acute regulatory protein), LBP (lipopolysaccharide binding protein), ABCC6 (ATP-binding cassette, sub-family C(CFTR/MRP), member 6), RGS2 (regulator of G-protein signaling 2, 24 kDa), EFNB2 (ephrin-B2), cystic fibrosis transmembrane conductance regulator (CFTR), GJB6 (gap junction protein, beta 6, 30 kDa), APOA2 (apolipoprotein A-II), AMPD1 (adenosine monophosphate deaminase 1), DYSF (dysferlin, limb girdle muscular dystrophy 2B (autosomal recessive)), FDFT1 (farnesyl-diphosphate farnesyltransferase 1), EDN2 (endothelin 2), CCR6 (chemokine (C-C motif) receptor 6), GJB3 (gap junction protein, beta 3, 31 kDa), IL1RL1 (interleukin 1 receptor-like 1), ENTPD1 (ectonucleoside triphosphate diphosphohydrolase 1), BBS4 (Bardet-Biedl syndrome 4), CELSR2 (cadherin, EGF LAG seven-pass G-type receptor 2 (flamingo homolog, Drosophila)), F1IR (Fll receptor), RAPGEF3 (Rap guanine nucleotide exchange factor (GEF) 3), HYAL1 (hyaluronoglucosaminidase 1), ZNF259 (zinc finger protein 259), ATOX1 (ATX1 antioxidant protein 1 homolog (yeast)), ATF6 (activating transcription factor 6), K¹HK (ketohexokinase (fructokinase)), SAT1 (spermidine/spermine Nl-acetyltransferase 1), GGFI (gamma-glutamyl hydrolase (conjugase, folylpolygammaglutamyl hydrolase)), TIMP4 (TIMP metallopeptidase inhibitor 4), SLC4A4 (solute carrier family 4, sodium bicarbonate cotransporter, member 4), PDE2A (phosphodiesterase 2 A, cGMP-stimulated), PDE3B (phosphodiesterase 3B, cGMP-inhibited), FADS1 (fatty acid desaturase 1), FADS2 (fatty acid desaturase 2), TMSB4X (thymosin beta 4, X-linked), TXNIP (thioredoxin interacting protein), LIMS1 (LIM and senescent cell antigen-like domains 1), RFIOB (ras homolog gene family, member B), LY96 (lymphocyte antigen 96), FOXOl (forkhead box 01), PNPLA2 (patatin-like phospholipase domain containing 2), TRH (thyrotropin-releasing hormone), GJC1 (gap junction protein, gamma 1, 45 kDa), SLC17A5 (solute carrier family 17 (anion/sugar transporter), member 5), FTO (fat mass and obesity associated), GJD2 (gap junction protein, delta 2, 36 kDa), PSRC1 (proline/serine-rich coiled-coil 1), CASP12 (caspase 12 (gene/pseudogene)), GPBAR1 (G protein-coupled bile acid receptor 1), PXK (PX domain containing serine/threonine kinase), IL33 (interleukin 33), TRIB1 (tribbles homolog 1 (Drosophila)), PBX4 (pre-B-cell leukemia homeobox 4), NUPRI (nuclear protein, transcriptional regulator, 1), 15-Sep(15 kDa selenoprotein), CILP2 (cartilage intermediate layer protein 2), TERC (telomerase RNA component), GGT2 (gamma-glutamyltransf erase 2), MT-COl (mitochondrially encoded cytochrome c oxidase I), UOX (urate oxidase, pseudogene), a CRISPR/Cas effector polypeptide, an enzymatically active CRISPR/Cas effector polypeptide (e.g., is capable of cleaving a target nucleic acid) and a CRISPR/Cas effector polypeptide that is not enzymatically active (e.g., does not cleave a target nucleic acid, but retains binding to the target nucleic acid). In some cases, the donor DNA encodes a wild-type version of any of the foregoing polypeptides; i.e., the donor DNA can encode a “normal” version that does not include a mutation(s) that results in reduced function, lack of function, or pathogenesis.

In some cases, the donor DNA comprises a nucleotide sequence encoding a fluorescent polypeptide. Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilised EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFPl, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrapel, mRaspberry, mGrape2, m PI urn (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, can be encoded.

In some cases, the donor DNA encodes an RNA, e.g., an siRNA, a microRNA, a short hairpin RNA (shRNA), an anti-sense RNA, a riboswitch, a ribozyme, an aptamer, a ribosomal RNA, a transfer RNA, and the like.

A donor DNA can include, in addition to a nucleotide sequence encoding one or more gene products (e.g., an RNA and/or a polypeptide), one or more transcriptional control elements, e.g., a promoter, an enhancer, and the like. In some cases, the transcriptional control element is inducible. In some cases, the promoter is reversible. In some cases, the transcriptional control element is constitutive. In some cases, the promoter is functional in a eukaryotic cell. In some cases, the promoter is a cell type-specific promoter. In some cases, the promoter is a tissue-specific promoter.

The nucleotide sequence of the donor DNA is typically not identical to the target nucleic acid (e.g., genomic sequence) that it replaces. Rather, the donor DNA may contain at least one or more single base changes, insertions, deletions, inversions or rearrangements with respect to the target nucleic acid (e.g., genomic sequence), so long as sufficient homology is present to support homology-directed repair (e.g., for gene correction, e.g., to convert a disease-causing base pair or a non-disease-causing base pair). In some cases, the donor DNA comprises a non-homologous sequence flanked by two regions of homology, such that homology-directed repair between the target DNA region and the two flanking sequences results in insertion of the non-homologous sequence at the target region. Donor DNA may also comprise a vector backbone containing sequences that are not homologous to the DNA region of interest (the target nucleic acid) and that are not intended for insertion into the DNA region of interest (the target nucleic acid). Generally, the homologous region(s) of a donor sequence will have at least 50% sequence identity to a target nucleic acid (e.g., a genomic sequence) with which recombination is desired. In certain cases, 60%, 70%, 80%, 90%, 95%, 98%, 99%, or 99.9% sequence identity is present. Any value between 1% and 100% sequence identity can be present, depending upon the length of the donor polynucleotide.

The donor DNA may comprise certain nucleotide sequence differences as compared to the target nucleic acid (e.g., genomic sequence), where such difference include, e.g. restriction sites, nucleotide polymorphisms, selectable markers (e.g., drug resistance genes, fluorescent proteins, enzymes etc.), etc., which may be used to assess for successful insertion of the donor DNA at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence. In some cases, the donor DNA will include one or more nucleotide sequences to aid in localization of the donor to the nucleus of the recipient cell or to aid in the integration of the donor DNA into the target nucleic acid. For example, in some case, the donor DNA may comprise one or more nucleotide sequences encoding one or more nuclear localization signals and the like (Frietas et al (2009) Cun-Genomics 10:550-7). In some cases, the donor DNA will include nucleotide sequences to recruit DNA repair enzymes to increase insertion efficiency. Fiuman enzymes involved in homology directed repair include MRN-CtIP, BLM-DNA2, Exol, ERCC1, Rad51, Rad52, Ligase 1, RoIQ, PARP1, Ligase 3, BRCA2, RecQ/BLM-ToroIIIa, RTEL, Roid, and Roih (Verma and Greenburg (2016) Genes Dev. 30 (10): 1138-1154). In some cases, the donor DNA is delivered as reconstituted chromatin (Cruz-Becerra and Kadonaga (2020) eLife 2020; 9:e55780 DOI: 10.7554/eLife.55780).

In some cases, the ends of the donor DNA are protected (e.g., from exonucleolytic degradation) by any convenient method and such methods are known to those of skill in the art. For example, one or more dideoxynucleotide residues can be added to the 3′ terminus of a linear molecule and/or self complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor DNA, additional lengths of sequence may be included outside of the regions of homology that can be degraded without impacting recombination.

### Linkers

In some embodiments, the polypeptides of the retron-based gene editing system are coupled to one another other to one or more accessory functions by a linker. Such accessory functions can include deaminases, nucleases, reverse transcriptases, and recombinases. For example, a retron RT may be linked to a programmable nuclease, such as, a Type II or Type V CRISPR nuclease. The term linker as used in reference to a fusion protein refers to a molecule which joins the proteins to form a fusion protein. Generally, such molecules have no specific biological activity other than to join or to preserve some minimum distance or other spatial relationship between the proteins. However, in one embodiment, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. Preferred peptide linker sequences adopt a flexible extended conformation and do not exhibit a propensity for developing an ordered secondary structure. In one embodiment, the linker can be a chemical moiety which can be monomeric, dimeric, multimeric or polymeric. Preferably, the linker comprises amino acids. Typical amino acids in flexible linkers include Gly, Asn and Ser.

Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180. For example, GlySer linkers may be based on repeating units of GGS, i.e., up to 2, 3, 4, 5, 6, 7, 8, 9, 10, 11 or even 12 or more repeating units, including but not limited to:

| SEQ ID | Description | Sequence |
|---|---|---|
| | GlySer linker based | GGS |
| | on GGS repeating | |
| | unit | |
| 19469 | GlySer linker based | GGS GGS |
| | on GGS repeating | |
| | unit | |
| 19470 | GlySer linker based | GGS GGS GGS |
| | on GGS repeating | |
| | unit | |
| 19471 | GlySer linker based | GGS GGS GGS GGS |
| | on GGS repeating | |
| | unit | |
| 19472 | GlySer linker based | GGS GGS GGS GGS GGS |
| | on GGS repeating | |
| | unit | |
| 19473 | GlySer linker based | GGS GGS GGS GGS GGS GGS |
| | on GGS repeating | |
| | unit | |
| 19474 | GlySer linker based | GGS GGS GGS GGS GGS GGS GGS |
| | on GGS repeating | |
| | unit | |
| 19475 | GlySer linker based | GGS GGS GGS GGS GGS GGS GGS GGS |
| | on GGS repeating | |
| | unit | |
| 19476 | GlySer linker based | GGS GGS GGS GGS GGS GGS GGS GGS GGS |
| | on GGS repeating | |
| | unit | |
| 19477 | GlySer linker based | GGS GGS GGS GGS GGS GGS GGS GGS GGS GGS |
| | on GGS repeating | |
| | unit | |
| 19478 | GlySer linker based | GGS GGS GGS GGS GGS GGS GGS GGS GGS GGS GGS |
| | on GGS repeating | |
| | unit | |
| 19479 | GlySer linker based | GGS GGS GGS GGS GGS GGS GGS GGS GGS GGS GGS GGS |
| | on GGS repeating | |
| | unit | |
| 19480 | GlySer linker based | GGS GGS GGS GGS GGS GGS GGS GGS GGS GGS GGS GGS |
| | on GGS repeating | GGS |
| | unit | |
| 19481 | GlySer linker based | GGS GGS GGS GGS GGS GGS GGS GGS GGS GGS GGS GGS |
| | on GGS repeating | GGS GGS |
| | unit | |
| 19482 | GlySer linker based | GGS GGS GGS GGS GGS GGS GGS GGS GGS GGS GGS GGS |
| | on GGS repeating | GGS GGS GGS |
| | unit | |

In another example, GlySer linkers may be based on repeating units of GSG, i.e., up to 2, 3, 4, 5, 6, 7, 8, 9, 10, 11 or even 12 or more repeating units, including but not limited to:

| SEQ | | |
|---|---|---|
| ID | Description | Sequence |
| | GlySer linker | GSG |
| | based on GSG | |
| | repeating unit | |
| 19483 | GlySer linker | GSG GSG |
| | based on GSG | |
| | repeating unit | |
| 19484 | GlySer linker | GSG GSG GSG |
| | based on GSG | |
| | repeating unit | |
| 19485 | GlySer linker | GSG GSG GSG GSG |
| | based on GSG | |
| | repeating unit | |
| 19486 | GlySer linker | GSG GSG GSG GSG GSG |
| | based on GSG | |
| | repeating unit | |
| 19487 | GlySer linker | GSG GSG GSG GSG GSG GSG |
| | based on GSG | |
| | repeating unit | |
| 19488 | GlySer linker | GSG GSG GSG GSG GSG GSG GSG GSG |
| | based on GSG | |
| | repeating unit | |
| 19489 | GlySer linker | GSG GSG GSG GSG GSG GSG GSG GSG GSG |
| | based on GSG | |
| | repeating unit | |
| 19490 | GlySer linker | GSG GSG GSG GSG GSG GSG GSG GSG GSG GSG |
| | based on GSG | |
| | repeating unit | |
| 19491 | GlySer linker | GSG GSG GSG GSG GSG GSG GSG GSG GSG GSG GSG |
| | based on GSG | |
| | repeating unit | |
| 19492 | GlySer linker | GSG GSG GSG GSG GSG GSG GSG GSG GSG GSG GSG GSG |
| | based on GSG | |
| | repeating unit | |
| 19493 | GlySer linker | GSG GSG GSG GSG GSG GSG GSG GSG GSG GSG GSG GSG GSG |
| | based on GSG | |
| | repeating unit | |
| 19494 | GlySer linker | GSG GSG GSG GSG GSG GSG GSG GSG GSG GSG GSG GSG GSG |
| | based on GSG | GSG |
| | repeating unit | |
| 19495 | GlySer linker | GSG GSG GSG GSG GSG GSG GSG GSG GSG GSG GSG GSG GSG |
| | based on GSG | GSG GSG |
| | repeating unit | |

In yet another example, GlySer linkers may be based on repeating units of GGGS, i.e., up to 2, 3, 4, 5, 6, 7, 8, 9, 10, 11 or even 12 or more repeating units, including but not limited to:

| SEQ | | |
|---|---|---|
| ID | Description | Sequence |
| 19496 | GlySer linker | GGGS |
| | based on GGGS | |
| | repeating unit | |
| 19497 | GlySer linker | GGGS GGGS |
| | based on GGGS | |
| | repeating unit | |
| 19498 | GlySer linker | GGGS GGGS GGGS |
| | based on GGGS | |
| | repeating unit | |
| 19499 | GlySer linker | GGGS GGGS GGGS GGGS |
| | based on GGGS | |
| | repeating unit | |
| 19500 | GlySer linker | GGGS GGGS GGGS GGGS GGGS |
| | based on GGGS | |
| | repeating unit | |
| 19501 | GlySer linker | GGGS GGGS GGGS GGGS GGGS GGGS |
| | based on GGGS | |
| | repeating unit | |
| 19502 | GlySer linker | GGGS GGGS GGGS GGGS GGGS GGGS GGGS |
| | based on GGGS | |
| | repeating unit | |
| 19503 | GlySer linker | GGGS GGGS GGGS GGGS GGGS GGGS GGGS GGGS |
| | based on GGGS | |
| | repeating unit | |
| 19504 | GlySer linker | GGGS GGGS GGGS GGGS GGGS GGGS GGGS GGGS GGGS |
| | based on GGGS | |
| | repeating unit | |
| 19505 | GlySer linker | GGGS GGGS GGGS GGGS GGGS GGGS GGGS GGGS GGGS GGGS |
| | based on GGGS | |
| | repeating unit | |
| 19506 | GlySer linker | GGGS GGGS GGGS GGGS GGGS GGGS GGGS GGGS GGGS GGGS |
| | based on GGGS | GGGS |
| | repeating unit | |
| 19507 | GlySer linker | GGGS GGGS GGGS GGGS GGGS GGGS GGGS GGGS GGGS GGGS |
| | based on GGGS | GGGS GGGS |
| | repeating unit | |
| 19508 | GlySer linker | GGGS GGGS GGGS GGGS GGGS GGGS GGGS GGGS GGGS GGGS |
| | based on GGGS | GGGS GGGS GGGS |
| | repeating unit | |
| 19509 | GlySer linker | GGGS GGGS GGGS GGGS GGGS GGGS GGGS GGGS GGGS GGGS |
| | based on GGGS | GGGS GGGS GGGS GGGS |
| | repeating unit | |
| 19510 | GlySer linker | GGGS GGGS GGGS GGGS GGGS GGGS GGGS GGGS GGGS GGGS |
| | based on GGGS | GGGS GGGS GGGS GGGS GGGS |
| | repeating unit | |

In still another example, GlySer linkers may be based on repeating units of GGGGS, i.e., up to 2, 3, 4, 5, 6, 7, 8, 9, 10, 11 or even 12 or more repeating units, including but not limited to:

| SEQ | | |
|---|---|---|
| ID | Description | Sequence |
| 19511 | GlySer linker | GGGGS |
| | based on | |
| | GGGGS | |
| | repeating unit | |
| 19512 | GlySer linker | GGGGS GGGGS |
| | based on | |
| | GGGGS | |
| | repeating unit | |
| 19513 | GlySer linker | GGGGS GGGGS GGGGS |
| | based on | |
| | GGGGS | |
| | repeating unit | |
| 19514 | GlySer linker | GGGGS GGGGS GGGGS GGGGS |
| | based on | |
| | GGGGS | |
| | repeating unit | |
| 19515 | GlySer linker | GGGGS GGGGS GGGGS GGGGS GGGGS |
| | based on | |
| | GGGGS | |
| | repeating unit | |
| 19516 | GlySer linker | GGGGS GGGGS GGGGS GGGGS GGGGS GGGGS |
| | based on | |
| | GGGGS | |
| | repeating unit | |
| 19517 | GlySer linker | GGGGS GGGGS GGGGS GGGGS GGGGS GGGGS GGGGS |
| | based on | |
| | GGGGS | |
| | repeating unit | |
| 19518 | GlySer linker | GGGGS GGGGS GGGGS GGGGS GGGGS GGGGS GGGGS GGGGS |
| | based on | |
| | GGGGS | |
| | repeating unit | |
| 19519 | GlySer linker | GGGGS GGGGS GGGGS GGGGS GGGGS GGGGS GGGGS GGGGS |
| | based on | GGGGS |
| | GGGGS | |
| | repeating unit | |
| 19520 | GlySer linker | GGGGS GGGGS GGGGS GGGGS GGGGS GGGGS GGGGS GGGGS |
| | based on | GGGGS GGGGS |
| | GGGGS | |
| | repeating unit | |
| 19521 | GlySer linker | GGGGS GGGGS GGGGS GGGGS GGGGS GGGGS GGGGS GGGGS |
| | based on | GGGGS GGGGS GGGGS |
| | GGGGS | |
| | repeating unit | |
| 19522 | GlySer linker | GGGGS GGGGS GGGGS GGGGS GGGGS GGGGS GGGGS GGGGS |
| | based on | GGGGS GGGGS GGGGS GGGGS |
| | GGGGS | |
| | repeating unit | |
| 19523 | GlySer linker | GGGGS GGGGS GGGGS GGGGS GGGGS GGGGS GGGGS GGGGS |
| | based on | GGGGS GGGGS GGGGS GGGGS GGGGS |
| | GGGGS | |
| | repeating unit | |
| 19524 | GlySer linker | GGGGS GGGGS GGGGS GGGGS GGGGS GGGGS GGGGS GGGGS |
| | based on | GGGGS GGGGS GGGGS GGGGS GGGGS GGGGS |
| | GGGGS | |
| | repeating unit | |
| 19525 | GlySer linker | GGGGS GGGGS GGGGS GGGGS GGGGS GGGGS GGGGS GGGGS |
| | based on | GGGGS GGGGS GGGGS GGGGS GGGGS GGGGS GGGGS |
| | GGGGS | |
| | repeating unit | |

In yet a further embodiment, LEPGEKPYKCPECGKSFSQSGALTRHQRTHTR (SEQ ID NO: 19526) is used as a linker.

In yet an additional embodiment, the linker is an XTEN linker, which is TCGGGATCTGAGACGCCTGGGACCTCGGAATCGGCTACGCCCGAAAGT (SEQ ID NO: 19527). In further particular embodiments, Cas12a polypeptide is linked C-terminally to the N-terminus of a deaminase protein or its catalytic domain by means of an LEPGEKPYKCPECGKSFSQSGALTRHQRTHTRLEPGEKPYKCPECGKSFSQSGALTRHQR THTRLEPGEKPYKCPECGKSFSQSGALTRHQRTHTR (SEQ ID NO: 19528) linker. In addition, N- and C-terminal NLSs can also function as linker (e.g., PKKKRKVEASSPKKRKVEAS (SEQ ID NO: 19420)).

The above description of linkers is intended to be non-limiting and includes any combinations of the above linkers or heterologous combinations of repeating GlySer linkers.

The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminoHEXAnoic acid (Ahx). In certain embodiments, the linker is based on a carbocyclic moiety (e.g., cyclopentane, cycloHEXAne). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may included functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

The linker can be, for example, a cleavable linker or protease-sensitive linker. In some embodiments, the linker is selected from the group consisting of F2A linker, P2A linker, T2A linker, E2A linker, and combinations thereof. This family of self-cleaving peptide linkers, referred to as 2A peptides, has been described in the art (see for example, Kim, J. H. et al. (2011) PLoS ONE 6:e18556). In some embodiments, the linker is an F2A linker. In some embodiments, the linker is a GGGS linker. In some embodiments, the fusion protein contains three domains with intervening linkers, having the structure: domain-linker-domain-linker-domain.

Cleavable linkers known in the art may be used in connection with the disclosure. Exemplary such linkers include: F2A linkers, T2A linkers, P2A linkers, E2A linkers (See, e.g., WO2017127750). The skilled artisan will appreciate that other art-recognized linkers may be suitable for use in the constructs of the disclosure (e.g., encoded by the nucleic acids of the disclosure). The skilled artisan will likewise appreciate that other polycistronic constructs (mRNA encoding more than one nucleobase editing system component/polypeptide separately within the same molecule) may be suitable for use as provided herein.

### Nuclear Localization Domains

In various embodiments, the gene editing systems or any of the components thereof may fused to one or more nuclear localization sequences (NLSs), such as about or more than about 1, 2, 3, 4, 5, 6, 7, 8, 9, 10, or more NLSs. In one embodiment, a gene editor component (e.g., a nucleic acid programmable DNA binding protein or an editing accessory protein) comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In an embodiment of the invention, an editor component polypeptide comprises at most 6 NLSs. In one embodiment, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Nonlimiting examples of NLSs include an NLS sequence derived from: the NLS of the SV40 virus large T-antigen, having the amino acid sequence PKKKRKV (SEQ ID NO: 19399); the NLS from nucleoplasmin (e.g. the nucleoplasmin bipartite NLS with the sequence KRPAATKKAGQAKKKK (SEQ ID NO: 19529); the c-myc NLS having the amino acid sequence PAAKRVKLD (SEQ ID NO: 19530) or RQRRNELKRSP (SEQ ID NO: 19531); the hRNPAl M9 NLS having the sequence NQSSNFGPMKGGNFGGRSSGPYGGGGQYFAKPRNQGGY (SEQ ID NO: 19532); the sequence RMRIZFKNKGKDTAELRRRRVEVSVELRKAKKDEQILKRRNV (SEQ ID NO: 19533) of the IBB domain from importin-alpha; the sequences VSRKRPRP (SEQ ID NO: 19400) and PPKKARED (SEQ ID NO: 19534) of the myoma T protein; the sequence PQPKKKPL (SEQ ID NO: 19535) of human p53; the sequence SALIKKKKKMAP (SEQ ID NO: 19536) of mouse c-abl IV; the sequences DRLRR (SEQ ID NO: 19537) and PKQKKRK (SEQ ID NO: 19538) of the influenza virus NS 1; the sequence RKLKKKIKKL (SEQ ID NO: 19539) of the Hepatitis virus delta antigen; the sequence REKKKFLKRR (SEQ ID NO: 19540) of the mouse Mxl protein; the sequence KRKGDEVDGVDEVAKKKSKK (SEQ ID NO: 19541) of the human poly(ADP-ribose) polymerase; and the sequence RI<CLQAGMNLEARI<TI<I<(SEQ ID NO: 19542) of the steroid hormone receptors (human) glucocorticoid.

In general, the one or more NLSs are of sufficient strength to drive accumulation of a polypeptide of interest (e.g., retron RT) in a detectable amount in the nucleus of a eukaryotic cell. In general, strength of nuclear localization activity may derive from the number of NLSs in the polypeptide of interest (e.g., retron RT), the particular NLS(s) used, or a combination of these factors. Detection of accumulation in the nucleus may be performed by any suitable technique.

For example, a detectable marker may be fused to a polypeptide of interest (e.g., retron RT), such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of complex formation (e.g., assay for DNA cleavage or mutation at the target sequence, or assay for altered gene expression activity affected by complex formation and/or polypeptide activity), as compared to a control no exposed to the polypeptide or complex, or exposed to a polypeptide lacking the one or more NLSs. In one embodiment of the herein described polypeptide protein complexes and systems the codon optimized polypeptide proteins comprise an NLS attached to the C-terminal of the protein. In one embodiment, other localization tags may be fused to the polypeptide, such as without limitation for localizing the polypeptide to particular sites in a cell, such as organelles, such as mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

In one embodiment of the invention, at least one nuclear localization signal (NLS) is attached to the nucleic acid sequences encoding the polypeptide of interest (e.g., a retron RT). In preferred embodiments at least one or more C-terminal or N-terminal NLSs are attached (and hence nucleic acid molecule(s) coding for the polypeptide can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

In other examples, the fusion proteins comprising a polypeptide of interest (e.g., retron RT) and another accessory protein (e.g., nuclease) contains one or more nuclear localization signals is selected or derived from SV40, c-Myc or NLP-1.

The NLS examples above are non-limiting. The proteins and/or fusions contemplated herein may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In various embodiments, any of the polypeptide components of the retron-based editing systems may be engineered with one or more nuclear localization signals, which help promote translocation of a protein into the cell nucleus. The polypeptides of the retron-based editing system may comprise any known NLS sequence, including any of those described in Cokol et al., “Finding nuclear localization signals,” EMBO Rep., 2000, 1(5): 411-415 and Freitas et al., “Mechanisms and Signals for the Nuclear Import of Proteins,” Current Genomics, 2009, 10(8): 550-7, each of which are incorporated herein by reference.

In various embodiments, the polypeptides disclosed herein may include one or more, preferably, at least two nuclear localization signals. The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations). The term“nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application PCT/EP2000/011690, filed Nov. 23, 2000, published as WO/2001/038547 on May 31, 2001, the contents of which are incorporated herein by reference. In some embodiments, an NLS comprises the amino acid sequence PKKKRKV (SEQ ID NO: 19399).

Another representative nuclear localization signal is a peptide sequence that directs the protein to the nucleus of the cell in which the sequence is expressed. A nuclear localization signal is predominantly basic, can be positioned almost anywhere in a protein's amino acid sequence, generally comprises a short sequence of four amino acids (Autieri & Agrawal, (1998) J. Biol. Chem. 273: 14731-37, incorporated herein by reference) to eight amino acids, and is typically rich in lysine and arginine residues (Magin et al., (2000) Virology 274: 11-16, incorporated herein by reference). Nuclear localization signals often comprise proline residues. A variety of nuclear localization signals have been identified and have been used to effect transport of biological molecules from the cytoplasm to the nucleus of a cell. See, e.g., Tinland et al., (1992) Proc. Natl. Acad. Sci. U.S.A. 89:7442-46; Moede et al., (1999) FEBS Lett.461:229-34, which is incorporated by reference. Translocation is currently thought to involve nuclear pore proteins.

Most NLSs can be classified in three general groups: (i) a monopartite NLS exemplified by the SV40 large T antigen NLS (PKKKRKV) (SEQ ID NO: 19399); (ii) a bipartite motif consisting of two basic domains separated by a variable number of spacer amino acids and exemplified by the Xenopus nucleoplasmin NLS (KRXXXXXXXXXXKKKL) (SEQ ID NO: 19419); and (iii) noncanonical sequences such as M9 of the hnRNP A1 protein, the influenza virus nucleoprotein NLS, and the yeast Gal4 protein NLS (Dingwall and Laskey 1991). Nuclear localization signals appear at various points in the amino acid sequences of proteins. NLS's have been identified at the N-terminus, the C-terminus and in the central region of proteins. Thus, the disclosure provides polypeptides that may be modified with one or more NLSs at the C-terminus, the N-terminus, as well as at in internal region of a polypeptide (including a fusion protein).

The present disclosure contemplates any suitable means by which to modify a polypeptide to include one or more NLSs. In one aspect, a polypeptide (e.g., a programmable nuclease) may be engineered to express with a translationally fused NLS at its N-terminus or its C-terminus (or both), i.e., to form a polypeptide-NLS fusion construct. In addition, the NLSs may include various amino acid linkers or spacer regions encoded between a polypeptide and the N-terminally, C-terminally, or internally-attached NLS amino acid sequence, e.g, and in the central region of proteins.

Thus, the present disclosure also provides for nucleotide constructs, vectors, and host cells for expressing fusion proteins that comprise a polypeptide and one or more NLSs.

### Tag Domains

In some embodiments, the retron-based editing system or a component thereof may comprise a polypeptide tag, such as an affinity tag (chitin binding protein (CBP), maltose binding protein (MBP), glutathione-S-transferase (GST), SBP-tag, Strep-tag, AviTag, Calmodulin-tag); solubilization tag; chromatography tag (polyanionic amino acid tag, such as FLAG-tag); epitope tag (short peptide sequences that bind to high-affinity antibodies, such as V5-tag, Myc-tag, VSV-tag, Xpress tag, E-tag, S-tag, and HA-tag); fluorescence tag (e.g., GFP). In some embodiments, the retron-based editing system peptide may comprise an amino acid tag, such as one or more lysines, histidines, or glutamates, which can be added to the polypeptide sequences (e.g., at the N-terminal or C-terminal ends). Lysines can be used to increase peptide solubility or to allow for biotinylation. Protein and amino acid tags are peptide sequences genetically grafted onto a recombinant protein. Sequence tags are attached to proteins for various purposes, such as peptide purification, identification, or localization, for use in various applications including, for example, affinity purification, protein array, western blotting, immunofluorescence, and immunoprecipitation. Such tags are subsequently removable by chemical agents or by enzymatic means, such as by specific proteolysis or intein splicing.

Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

### Aptamers

In particular embodiments, the nucleic acid components (e.g., guide RNAs or retron ncRNAs) of the retron-based editing systems may further comprise a functional structure designed to improve nucleic acid component molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Sarnie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jiehua, and John J. Rossi. “Aptamer-targeted cell-specific RNA interference.” Silence 1.1 (2010): 4).

Accordingly, in particular embodiments, a retron-based gene editing nucleic acid component is modified, e.g., by one or more aptamer(s) designed to improve RNA or DNA component molecule delivery, including delivery across the cellular membrane, to intracellular compartments, or into the nucleus. Such a structure can include, either in addition to the one or more aptamer(s) or without such one or more aptamer(s), moiety(ies) so as to render the nucleic acid component molecule deliverable, inducible or responsive to a selected effector. The invention accordingly comprehends a reRNA component molecule that responds to normal or pathological physiological conditions, including without limitation pH, hypoxia, oxygen concentration, temperature, protein concentration, enzymatic concentration, lipid structure, light exposure, mechanical disruption (e.g. ultrasound waves), magnetic fields, electric fields, or electromagnetic radiation.

Agents that Modulate DNA-Repair

In certain embodiments, the retron-based gene editing systems described herein further comprises or encodes a DNA-repair modulating biomolecule, which may further enhance the efficiency of integration of a repair outcome.

In certain embodiments, the DNA-repair modulating biomolecule comprises a Nonhomologous end joining (NHEJ) inhibitor.

In certain embodiments, the DNA-repair modulating biomolecule comprises a homologous directed repair (HDR) promoter.

In certain embodiments, the DNA-repair modulating biomolecule comprises a NHEJ inhibitor and an HDR promoter.

In certain embodiments, the DNA-repair modulating biomolecule enhances or improves more precise genome editing and/or the efficiency of homologous recombination, compared to the otherwise identical embodiment without the DNA-repair modulating biomolecule.

HDR promoters and/or NHEJ inhibitors can, in some embodiments, comprise one or more small molecules. Systems bearing recombination enhancers such as small molecules that activate HDR and suppress NHEJ locally at the genomic site of the DNA damage can be tailored in their placement on the engineered systems to further enhance their efficiency. In general, the small molecule recombination enhancers can be synthesized to bear linkers and a functional group, such as maleimide for reacting with a thiol group on a Cys residue of a protein, for chemical conjugation to the engineered systems. Use of commercially available functionalized PEG linkers (alkyne, azide, cyclooctyne etc.) can also be employed for conjugation, and orthogonal conjugation chemistries can be utilized for the multivalent display.

Conjugation sites can be readily identified where modifications do not affect the potency of the recombination enhancers selected.

In certain embodiments, multivalent display of one or more DNA-repair modulating biomolecule can be effected, including multiple moieties of NHEJ inhibitors, HDR promoters, or a combination thereof. See, for example, “Genomic targeting of epigenetic probes using a chemically tailored Cas9 system” by Liszczak et al., Proc Natl Acad Sci U.S.A. 114: 681-686, 2017 (incorporated herein by reference). In certain embodiments, multivalent display of small molecule compounds can be achieved through sortase loop proteins used as a scaffold for their display.

In some embodiments, the DNA-repair modulating biomolecule may comprise an HDR promoter. The HDR promoter may comprise small molecules, such as RSI or analogs thereof. In certain embodiments, the HDR promoter stimulates RAD51 activity or RAD52 motif protein 1 (RDM1) activity. In certain embodiments, the HDR promoter comprises Nocodazole, which can result in higher HDR selection.

In certain embodiments, the HDR promoter may be administered prior to the delivery of the retron-based editing systems described herein.

In certain embodiments, the HDR promoter locally enhances HDR without NHEJ inhibition. For example, RAD5l is a protein involved in strand exchange and the search for homology regions during HDR repair. In certain embodiments, the HDR promoter is phenylbenzamide RSI, identified as a small-molecule RAD51-stimulator (see WO2019/135816 at [0200]-[0204], specifically incorporated herein by reference).

In certain embodiments, the DNA-repair modulating biomolecule comprises C-terminal binding protein interacting protein (CtIP) or a functional fragment or homolog thereof. CtIP is a key protein in early steps of homologous recombination. According to this embodiment, the CtIP or the functional fragment or homolog thereof can be linked (e.g., fused) to the RT or the sequence-specific nuclease (e.g., a CRISPR/Cas effector enzyme, a ZFN, a TALEN, a meganuclease, TnpB, IscB, or a restriction endonuclease (RE)), and stimulates transgene integration by HDR.

In certain embodiments, the CtIP fragment is a minimal N-terminal fragment of the wild-type CtIP, such as the N-terminal fragment comprising residues 1-296 of the full-length CtIP (the HE for HDR enhancer), as described in Charpentier et al. (Nature Comm., DOI: 10.1038/s41467-018-03475-7, incorporated herein by reference), shown to be sufficient to stimulate HDR. The activity of the fragment depends on CDK phosphorylation sites (e.g., S233, T245, and S276) and the multimerization domain essential for CtIP activity in homologous recombination. Thus alternative fragments comprising the CDK phosphorylation sites and the multimerization domain essential for CtIP activity are also within the scope of the invention.

In certain embodiments, the DNA-repair modulating biomolecule comprises a dominant negative 53BP1.

In certain embodiments, the DNA-repair modulating biomolecule comprises a cell cycle-specific degradation tag, such as the degradation domain of the (human) Geminin, and the (murine) CyclinB2.

In certain embodiments, the DNA-repair modulating biomolecule comprises CyclinB2, a member of the B-type cyclins that associate with p34cdc2, and an essential component of the cell cycle regulatory machinery. CRISPR-mediated knock-in efficiency may be increased by promoting the relative increase in Cas9 activity in G2 phase of the cell cycle, when HDR is more active. In certain embodiments, the degradation domains of the (human) Geminin and (murine) CyclinB2 can be used as either N- or C-terminal fusion to serve as the DNA-repair modulating biomolecule. These domains are known to determine a cell-cycle specific profile of chimeric proteins, namely an increase in their relative concentration in S and G2 compared to G1, high-jacking the conventional CyclinB2 and Geminin degradation pathways. This produces active Geminin-Cas9 and CyclinB2-Cas9 chimeric proteins, which are degraded in a cell-cycle-dependent manner. Such chimeras shift the repair of the DSBs to the HDR repair pathway compared to the commonly used Cas9.

While not wishing to be bound by particular theory, it is believed that the application of such cell cycle-specific degradation tags permits/promotes more efficient/secure gene editing.

In certain embodiments, the DNA-repair modulating biomolecule comprises a Rad family member protein, such as Rad50, Rad51, Rad52, etc., which functions to promote foreign DNA integration into a host chromosome. Specifically, Rad52 is an important homologous recombinant protein, and its complex with Rad51 plays a key role in HDR, mainly involved in the regulation of foreign DNA in eukaryotes. Key steps in the process of HR include repair mediated by Rad51 and strand exchange. Co-expression of Rad52 as a DNA-repair modulating biomolecule significantly enhances the likelihood of HDR by, e.g., three-fold.

In certain embodiments, the DNA-repair modulating biomolecule comprises a RAD52 protein as, e.g., either an N- or a C-terminal fusion.

In certain embodiments, the DNA-repair modulating biomolecule comprises a RAD52 motif protein 1 (RDM1) that functions similarly as RAD52. RDM1 has been shown to be able to repair DSBs caused by DNA replication, prevent G2 or M cell cycle arrest, and improve HDR selection.

In certain embodiments, the DNA-repair modulating biomolecule comprises a dominant negative version of the tumor suppressor p53-binding protein 1 (53BP1). The wild-type protein 53BP1 is a key regulator of the choice between NHEJ and HDR—it is a pro-NHEJ factor which limits HDR by blocking DNA end resection, and also by inhibiting BRCA1 recruitment to DSB sites. It has been shown that global inhibition of 53BP1 by a ubiquitin variant significantly improves Cas9-mediated HDR frequency in non-hematopoietic and hematopoietic cells with single-strand oligonucleotide delivery or double-strand donor in AAV.

In certain embodiments, the dominant negative (DN) version of the 53BP1 comprises the minimal focus forming region, but lacks domains outside this region, e.g., towards the N-terminus and tandem C-terminal BRCT repeats that recruit key effectors involved in NHEJ, such as RIF1-PTIP and EXPAND, respectively. The 53BP1 adapter protein is recruited to specific histone marks at sites of DSBs via this minimal focus forming region, which comprises several conserved domains including an oligomerization domain (OD), a glycine-arginine rich (GAR) motif, a Tudor domain, and an adjacent ubiquitin-dependent recruitment (UDR) motif. The Tudor domain mediates interactions with histone H4 dimethylated at K2023.

In certain embodiments, a dominant negative version of 53BP1 (DN1S) suppresses the accumulation of endogenous 53BP1 and downstream NHEJ proteins at sites of DNA damage, while upregulating the recruitment of the BRCA1 HDR protein. Such a DN version of the 53BP1 can be used as the DNA-repair modulating biomolecule, either as an N- or a C-terminal fusion (such as a Cas9 fusion, to locally inhibit NHEJ at the Cas9-target site defined by its gRNA, while promoting an increase in HDR, and does not globally affect NHEJ, thereby improving cell viability).

In certain embodiments, the DNA-repair modulating biomolecule comprises an NHEJ inhibitor, such as an inhibitor of DNA ligase IV, a KU inhibitor (e.g., KU70 or KU80), a DNA-PKc inhibitor, or an artemis inhibitor.

In certain embodiments, the NHEJ inhibitor inhibits the NHEJ pathway, enhances HDR, or modulates both. In certain embodiments, the NHEJ inhibitor is a small molecule inhibitor.

In certain embodiments, the small molecule inhibitor of the NHEJ pathway comprises an SCR7 analog, for example, PK66, PK76, PK409.

In certain embodiments, the NHEJ inhibitor comprises a KU inhibitor, for example, KU5788, and KU0060648.

In certain embodiments, a small molecule NHEJ inhibitor is linked to a polyglycine tripeptide through PEG for sortase-mediated ligation, as described in WO2019/135816, Guimaraes et al., Nat Protoc 8:1787-99, 2013; Theile et al., Nat Protoc 8:1800-7, 2013; and Schmohl et al., Curr Opin Chem Biol 22:122-8, 2014 (all incorporated herein by reference). The same means can also be used for attaching small molecule HDR enhancers to protein.

An exemplary method for conjugating a small molecule DNA-repair modulating biomolecule without loss of activity is described in WO2019135816, where SCR-7 conjugation of a poly-glycine peptide with the para-carboxylic moiety at ring 4 retained activity of the inhibitor, with rings 1, 2 and 3 of the molecule having involvement in the target-engagement, providing a simple and effective strategy to ligate a small molecule NHEJ inhibitor to the system described herein (e.g., to the sequence-specific nuclease including Cas enzymes, or to the RT) to precisely enhance HDR pathway near a nucleic acid target site.

In certain embodiments, a nucleic acid targeting moiety conjugates based on small molecule inhibitor of DNA-dependent protein kinase (DNA-PK) or heterodimeric Ku (KU70/KU80) can be utilized. KU-0060648 is one potent KU-inhibitors, which can also be functionalized with poly-glycine and used for recombination enhancement.

In certain embodiments, the DNA-repair modulating biomolecule comprises the Tumor Suppressor p53. p53 plays a direct role in DNA repair, including HR regulation, where it affects the extension of new DNA, thereby affecting HDR selection. In vivo, p53 binds to the nuclear matrix and is a rate-limiting factor in repairing DNA structure. p53 regulates DNA repair processes in almost all eukaryotes via transactivation-dependent and -independent pathways, but only the transactivation-independent function of p53 is involved in HR regulation. Wild-type p53 protein can link double stranded breaks to form intact DNA, as well as also playing a role in inhibiting NHEJ. p53 interacts with HR-related proteins, including Rad51, where it controls HR through direct interaction with Rad51.

### Accessory Domains

In other aspects, the retron-based gene editing systems may comprise one or more additional accessory proteins having genome modifying functions, including recombinases, invertases, nucleases, polymerases, ligases, deaminases, reverse transcriptases, or epigenetic modifying functions. In various embodiments, the accessory proteins may be provided separately. In other embodiments, the accessory proteins may be fused to a polypeptide component of the retron-based editing systems, optionally with a linker.

The retron-based gene editing systems may further comprise additional polypeptides, proteins and/or peptides known in the art. Non-limiting categories of polypeptides include antigens, antibodies, antibody fragments, cytokines, peptides, hormones, enzymes, oxidants, antioxidants, synthetic polypeptides, and chimeric polypeptides, receptor, enzymes, hormones, transcription factors, ligands, membrane transporters, structural proteins, nucleases, or a component, variant or fragment (e.g., a biologically active fragment) thereof.

As used herein, the term “peptide” generally refers to shorter polypeptides of about 50 amino acids or less. Peptides with only two amino acids may be referred to as “dipeptides.” Peptides with only three amino acids may be referred to as “tripeptides.” Polypeptides generally refer to polypeptides with from about 4 to about 50 amino acids. Peptides may be obtained via any method known to those skilled in the art. In some embodiments, peptides may be expressed in culture. In some embodiments, peptides may be obtained via chemical synthesis (e.g., solid phase peptide synthesis).

In some embodiments, the RNA payloads (e.g., linear and/or circular mRNA payloads encoding one or more encoded products of interest or the non-coding RNAs such as guide RNAs) may encode a user-programmable DNA binding protein, or a gene editor accessory proteins, such as, but not limited to a deaminases, nucleases, transposases, polymerases, and reverse transcriptases, etc.

In some embodiments, the RNA payloads (e.g., linear and/or circular mRNA payloads encoding one or more encoded products of interest), e.g., the originator constructs and benchmark constructs described herein, may encode a simple protein associated with a non-protein. Non-limiting examples of conjugated proteins include, glycoproteins, hemoglobins, lecithoproteins, nucleoproteins, and phosphoproteins.

In some embodiments, the RNA payloads (e.g., linear and/or circular mRNA payloads encoding one or more encoded products of interest), e.g., the originator constructs and benchmark constructs described herein, may encode a protein that is derived from a simple or conjugated protein by chemical or physical means. Non-limiting examples of derived proteins include denatured proteins and peptides.

In some embodiments, the polypeptide, protein or peptide may be unmodified.

In some embodiments, the polypeptide, protein or peptide may be modified. Types of modifications include, but are not limited to, phosphorylation, glycosylation, acetylation, ubiquitylation/sumoylation, methylation, palmitoylation, quinone, amidation, myristoylation, pyrrolidone carboxylic acid, hydroxylation, phosphopantetheine, prenylation, GPI anchoring, oxidation, ADP-ribosylation, sulfation, S-nitrosylation, citrullination, nitration, gamma-carboxyglutamic acid, formylation, hypusine, topaquinone (TPQ), bromination, lysine topaquinone (LTQ), tryptophan tryptophylquinone (TTQ), iodination, and cysteine tryptophylquinone (CTQ). In some aspects, the polypeptide, protein or peptide may be modified by a post-transcriptional modification which can affect its structure, subcellular localization, and/or function.

In some embodiments, the polypeptide, protein or peptide may be modified using phosphorylation. Phosphorylation, or the addition of a phosphate group to serine, threonine, or tyrosine residues, is one of most common forms of protein modification. Protein phosphorylation plays an important role in fine tuning the signal in the intracellular signaling cascades.

In some embodiments, the polypeptide, protein or peptide may be modified using ubiquitination which is the covalent attachment of ubiquitin to target proteins. Ubiquitination-mediated protein turnover has been shown to play a role in driving the cell cycle as well as in protein-degradation-independent intracellular signaling pathways.

In some embodiments, the polypeptide, protein or peptide may be modified using acetylation and methylation which can play a role in regulating gene expression. As a non-limiting example, the acetylation and methylation could mediate the formation of chromatin domains (e.g., euchromatin and heterochromatin) which could have an impact on mediating gene silencing.

In some embodiments, the polypeptide, protein or peptide may be modified using glycosylation. Glycosylation is the attachment of one of a large number of glycan groups and is a modification that occurs in about half of all proteins and plays a role in biological processes including, but not limited to, embryonic development, cell division, and regulation of protein structure. The two main types of protein glycosylation are N-glycosylation and O-glycosylation. For N-glycosylation the glycan is attached to an asparagine and for O-glycosylation the glycan is attached to a serine or threonine.

In some embodiments, the polypeptide, protein or peptide may be modified using sumoylation. Sumoylation is the addition of SUMOs (small ubiquitin-like modifiers) to proteins and is a post-translational modification similar to ubiquitination.

In other embodiments, the RNA payloads (e.g., linear and/or circular mRNA payloads encoding one or more encoded products of interest), e.g., the originator constructs and benchmark constructs described herein, may encode a therapeutic protein, such as those exemplified below.

In other embodiments, the RNA payloads (e.g., linear and/or circular mRNA payloads encoding one or more products of interest), e.g., the originator constructs and benchmark constructs described herein, may encode a gene editing system, such as those exemplified herein. As used herein, a “nucleobase editing system” is a protein, DNA, or RNA composition capable of making edits, modifications or alterations to one or more targeted genes of interest. According to the present invention, one or more nucleobase editing system currently being marketed or in development may be encoded by the RNA payloads (e.g., linear and/or circular mRNA payloads encoding one or more encoded products of interest) described herein of the present invention.

### Inducibility Modifications

In one embodiment, a retron-based editing system may be configured as an inducible gene editing system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the retron-based editing system or component thereof may include a Light Inducible Transcriptional Effector (LITE) to direct changes in transcriptional activity in a sequence-specific manner. The components of a light may include a retron-editing system component, a light-responsive cytochrome heterodimer (e.g. from Arabidopsis thaliana), and a transcriptional activation/repression domain. Further examples of inducible DNA binding proteins and methods for their use are provided in US Provisional Application Nos. 61/736,465 and U.S. 61/721,283, and International Patent Publication No. WO 2014/018423 A2 which is hereby incorporated by reference in its entirety.

Once all copies of a gene in the genome of a cell have been edited, continued expression of the system in that cell is no longer necessary. Indeed, sustained expression would be undesirable in case of off-target effects at unintended genomic sites, etc. Thus time-limited expression would be useful. Inducible expression offers one approach, but in addition Applicants have engineered a self-inactivating system that relies on the use of a non-coding nucleic acid component molecule target sequence within the vector itself. Thus, after expression begins, the system will lead to its own destruction, but before destruction is complete it will have time to edit the genomic copies of the target gene (which, with a normal point mutation in a diploid cell, requires at most two edits). Simply, the self-inactivating system includes additional RNA (e.g., nucleic acid component molecule) that targets the coding sequence for the Cas12a polypeptide itself or that targets one or more non-coding nucleic acid component molecule target sequences complementary to unique sequences present in one or more of the following: (a) within the promoter driving expression of the non-coding RNA elements, (b) within the promoter driving expression of the Cas12a polypeptide gene, (c) within 100 bp of the ATG translational start codon in the Cas12a polypeptide coding sequence, (d) within the inverted terminal repeat (iTR) of a viral delivery vector, e.g., in the AAV genome.

In some aspects, a single nucleic acid component molecule is provided that is capable of hybridization to a sequence downstream of a Cas12a polypeptide start codon, whereby after a period of time there is a loss of the Cas12a polypeptide expression. In some aspects, one or more nucleic acid component molecule(s) are provided that are capable of hybridization to one or more coding or non-coding regions of the polynucleotide encoding the system, whereby after a period of time there is a inactivation of one or more, or in some cases all, of the system. In some aspects of the system, and not to be limited by theory, the cell may comprise a plurality of complexes, wherein a first subset of complexes comprise a first nucleic acid component molecule capable of targeting a genomic locus or loci to be edited, and a second subset of complexes comprise at least one second nucleic acid component molecule capable of targeting the polynucleotide encoding the system, wherein the first subset of complexes mediate editing of the targeted genomic locus or loci and the second subset of complexes eventually inactivate the system, thereby inactivating further expression in the cell.

The various coding sequences (Cas12a polypeptide and nucleic acid component molecule) can be included on a single vector or on multiple vectors. For instance, it is possible to encode the enzyme on one vector and the various RNA sequences on another vector, or to encode the enzyme and one nucleic acid component molecule on one vector, and the remaining nucleic acid component molecule on another vector, or any other permutation. In general, a system using a total of one or two different vectors is preferred.

### H. Vectors for Expression of Engineered Retrons

Delivery of an engineered retron to a cell can generally be accomplished with or without vectors. Delivery of the ncRNA encoded by the engineered retron generally does not require a vector used to produce the ncRNA from the engineered retron. For example, the ncRNA can be packaged directly into a delivery vehicle such as a lipid nanoparticle and delivered into a host cell, as described in other sections.

The engineered retrons (or vectors containing them) may be introduced into any type of cell, including any cell from a prokaryotic, eukaryotic, or archaeon organism, including bacteria, archaea, fungi, protists, plants (e.g., monocotyledonous and dicotyledonous plants); and animals (e.g., vertebrates and invertebrates). Examples of animals that may be transfected with an engineered retron include, without limitation, vertebrates such as fish, birds, mammals (e.g., human and non-human primates, farm animals, pets, and laboratory animals), reptiles, and amphibians.

The engineered retrons can be introduced into a single cell or a population of cells. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be transfected with the engineered retrons.

The engineered retrons can be introduced into cellular fragments, cell components, or organelles (e.g., mitochondria in animal and plant cells, plastids (e.g., chloroplasts) in plant cells and algae).

Cells may be cultured or expanded after transfection with the engineered retron.

Methods of introducing nucleic acids into a host cell are well known in the art. Commonly used methods include chemically induced transformation, typically using divalent cations (e.g., CaCl₂)), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising engineered retrons into nuclei. See, e.g., Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197; herein incorporated by reference in their entireties.

Methods for genetic transformation of plant cells are known in the art and include those set forth in US2022/0145296, and U.S. Pat. Nos. 8,575,425; 7,692,068; 8,802,934; 7,541,517; each of which is herein incorporated by reference in its entirety. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; and Jones et al. (2005) Plant Methods 1:5.

The plant cells that have been transformed may be grown into a transgenic organism, such as a plant, in accordance with conventional methods. See, for example, McCormick et al. (1986) Plant Cell Reports 5:81-84.

Plant material that may be transformed with the engineered retrons described herein includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Progeny, variants, and mutants of the regenerated plants are also included within the scope of the disclosure, provided that these parts comprise the genetic modification introduced by the engineered retron. Further provided is a processed plant product or byproduct that retains the genetic modification introduced by the engineered retron.

The engineered retrons described herein may be used to produce transgenic plants with desired phenotypes, including but not limited to, increased disease resistance (e.g., increased viral, bacterial of fungal resistance), increased insect resistance, increased drought resistance, increased yield, and altered fruit ripening characteristics, sugar and oil composition, and color.

In some embodiments, the retron msr gene, msd gene, and/or ret gene are expressed in vitro from a vector, such as in an in vitro transcription system. The resulting ncRNA or msDNA can be isolated before being packaged and/or formulated for direct delivery into a host cell. For example, the isolated ncRNA or msDNA can be packaged/formulated in a delivery vehicle such as lipid nanoparticles as described in other sections.

In some embodiments, the retron msr gene, msd gene, and/or ret gene are expressed in vivo from a vector within a cell. The retron msr gene, msd gene, and/or ret gene can be introduced into a cell with a single vector or in multiple separate vectors to produce msDNA in a host subject.

In other embodiments, the retron msr gene, msd gene, and/or ret gene, and any other components of the retron-based genome editing systems described herein (e.g., guide RNA in trans, programmable nuclease (e.g., in trans)) may be expressed in vivo from RNA delivered to the cell. The retron msr gene, msd gene, and/or ret gene can be introduced into a cell with a single vector or in multiple separate vectors to produce msDNA in a host subject.

Vectors and/or nucleic acid molecules encoding the recombinant retron-based genome editing system or components thereof can include control elements operably linked to the retron sequences, which allow for the production of msDNA either in vitro, or in vivo in the subject species. For example, the retron msr gene, msd gene, and/or ret gene can be operably linked to a promoter to allow expression of the retron reverse transcriptase and/or the msDNA product. In some embodiments, heterologous sequences encoding desired products of interest (e.g., polynucleotide encoding polypeptide or regulatory RNA, donor polynucleotide for gene editing, or protospacer DNA for molecular recording) may be inserted in the msr gene and/or msd gene.

Any eukaryotic, archaeal, or prokaryotic cell, capable of being transfected with a vector or retron delivery system comprising the engineered retron sequences, may be used to produce the msDNA in vivo. The ability of constructs to produce the msDNA along with other retron-encoded products can be empirically determined. For example the transfected cell can be assayed either through phenotypic changes that occur due to the introduced sequences or by direct DNA sequencing.

In some embodiments, the engineered retron is produced by a vector system comprising one or more vectors. In the vector system, the msr gene, the msd gene, and/or the ret gene may be provided by the same vector (i.e., cis arrangement of all such retron elements), wherein the vector comprises a promoter operably linked to the msr gene and/or the msd gene. In some embodiments, the promoter is further operably linked to the ret gene. In other embodiments, the vector further comprises a second promoter operably linked to the ret gene. Alternatively, the ret gene may be provided by a second vector that does not include the msr gene and/or the msd gene (i.e., trans arrangement of msr-msd and ret). In yet other embodiments, the msr gene, the msd gene, and the ret gene are each provided by different vectors (i.e., trans arrangement of all retron elements).

Numerous vectors are available for use in the vector or vector system, including but not limited to, linear polynucleotides, polynucleotides associated with ionic or amphiphilic compounds, plasmids, and viruses.

Examples of viral vectors include, but are not limited to, adenoviral vectors, adeno-associated virus (AAV) vectors, retroviral vectors, lentiviral vectors, and the like. An expression construct can be replicated in a living cell, or it can be made synthetically.

In some embodiments, the nucleic acid comprising an engineered retron sequence is under transcriptional control of a promoter. In some embodiments, the promoter is competent for initiating transcription of an operably linked coding sequence by a RNA polymerase 1, 11, or III.

Exemplary promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression.

Exemplary promoters for plant cell expression include the CaMV 35S promoter (Odell et al., 1985, Nature 313:810-812); the rice actin promoter (McElroy et al., 1990, Plant Cell 2:163-171); the ubiquitin promoter (Christensen et al., 1989, Plant Mol. Biol. 12:619-632; and Christensen et al., 1992, Plant Mol. Biol. 18:675-689); the pEMU promoter (Last et al., 1991, Theor. Appl. Genet. 81:581-588); and the MAS promoter (Velten et al., 1984, EMBO J. 3:2723-2730).

In additional embodiments, the retron-based vectors may also comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-b promoter, Mb promoter, Nphsl promoter, OG-2 promoter, SP-B promoter, SYN1 promoter, and WASP promoter.

These and other promoters can be obtained from or incorporated into commercially available plasmids, using techniques well known in the art. See, e.g., Sambrook et al., supra.

In some embodiments, one or more enhancer elements is/are used in association with the promoter to increase expression levels of the constructs. Examples include the SV40 early gene enhancer, as described in Dijkema et al., EMBOJ (1985) 4:761, the enhancer/promoter derived from the long terminal repeat (LTR) of the Rous Sarcoma Virus, as described in Gorman et al., Proc. Natl. Acad. Sci. USA (1982b) 79:6777, and elements derived from human CMV, as described in Boshart et al., Cell (1985) 41:521, such as elements included in the CMV intron A sequence. All such sequences are incorporated herein by reference.

In one embodiment, an expression vector for expressing an engineered retron, including the msr gene, msd gene, and/or ret gene comprises a promoter operably linked to a polynucleotide encoding the msr gene, msd gene, and/or ret gene.

In some embodiments, the vector or vector system also comprises a transcription terminator/polyadenylation signal. Examples of such sequences include, but are not limited to, those derived from SV40, as described in Sambrook et al., supra, as well as a bovine growth hormone terminator sequence (see, e.g., U.S. Pat. No. 5,122,458).

Additionally, 5′-UTR sequences can be placed adjacent to the coding sequence to further enhance the expression. Such sequences may include UTRs comprising an internal ribosome entry site (IRES). Inclusion of an IRES permits the translation of one or more open reading frames from a vector. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298: Rees et al., BioTechniques (1996) 20:102-110; Kobayashi et al., BioTechniques (1996) 21:399-402; and Mosser et al., BioTechniques (199722 ISO-161)c. A multitude of IRES sequences are known and include sequences derived from a wide variety of viruses, such as from leader sequences of picomaviruses such as the encephalomyocarditis virus (EMCV) UTR (Jang et al. Virol. (1989) 63:1651-1660). the polio leader sequence, the hepatitis A virus leader, the hepatitis C virus TRES, human rhinovirus type 2 IRES (Dobrikova et al., Proc. Natl. Acad. Sci. (2003) 100(251:15125-151301)). an IRES element from the foot and mouth disease virus (Ramesh et al., Nucl. Acid Res. (1996) 24:2697-2700), a giardiavirus IRES (Garlapati et al., J Biol. Chem. (2004) 279(51):3389-33971) and the like. A variety of nonviral IRES sequences will also find use herein, including, but not limited to IRES sequences from yeast, as well as the human angiotensin 11 type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17): 7622-7635), vascular endothelial growth factor IRES (Baranick et al. (2008) Proc. Natl. Acad Sci. U.S.A. 105(12):4733-4738, Stein et al. (1998) Mol. Cell. Biol. 18(6):3112-3119, Bert et al. (2006) RNA 12(6): 1074-1083), and insulin-like growth factor 2 IRES (Pedersen et al. (2002) Biochem. J. 363(Pt 1):37-44).

These elements are commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express multiple bacteriophage recombination proteins for recombineering or an RNA-guided nuclease (e.g., Cas9) for HDR in combination with a retron reverse transcriptase from an expression cassette.

In some embodiments, a polynucleotide encoding a viral self-cleaving 2A peptide, such as a T2A peptide, can be used to allow production of multiple protein products (e.g., Cas9, bacteriophage recombination proteins, retron reverse transcriptase) from a single vector or a single transcription unit under one promoter. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Jhosea asigna virus and porcine teschovirus-1. See, e.g., Kim et al. (2011) PLoS One 6(4): e18556, Trichas et al. (2008) BMC Biol. 6:40, Provost et al. (2007) Genesis 45(10): 625-629, Furler et al. (2001) Gene Ther. 8(11):864-873; herein incorporated by reference in their entireties.

In some embodiments, the expression construct comprises a plasmid suitable for transforming a bacterial host. Numerous bacterial expression vectors are known to those of skill in the art, and the selection of an appropriate vector is a matter of choice. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31 Bacterial plasmids may contain antibiotic selection markers (e.g., ampicillin, kanamycin, erythromycin, carbenicillin, streptomycin, or tetracycline resistance), a lacZ gene (b-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, METIS, ura4+, leul+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E coif) and yeast cells. A number of different types of yeast plasmids are available including yeast integrating plasmids (Yip), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct does not comprise a plasmid suitable for transforming a yeast cell.

In other embodiments, the expression construct comprises a virus or engineered construct derived from a viral genome. A number of viral based systems have been developed for gene transfer into mammalian cells. These include adenoviruses, retroviruses (g-retroviruses and lentiviruses), poxviruses, adeno-associated viruses, baculoviruses, and herpes simplex viruses (see e.g., Wamock et al. (2011) Methods Mol. Biol. 737:1-25; Walther et al. (2000) Drugs 60(2):249-271; and Lundstrom (2003) Trends Biotechnol. 21(3): 117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Bums et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24): 2516-2527). Lentiviruses are a class of retroviruses that are particularly useful for delivering polynucleotides to mammalian cells because they are able to infect both dividing and nondividing cells (see e.g., Lois et al. (2002) Science 295:868-872; Durand et al. (2011) Viruses 3(2): 132-159; herein incorporated by reference).

A number of adenoviral vectors have also been described. Unlike retroviruses which integrate into the host genome, adenoviruses persist extrachromosomally thus minimizing the risks associated with insertional mutagenesis.

Additionally, various adeno-associated vims (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor LaboratoryPress); Carter, B. J. Current Opinion in Biotechnology (1992) 3:533-539; Muzyczka, N. Current Topics in Microbiol and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the engineered retrons is the enterically administered recombinant poxvirus vaccines described by Small, Jr., P. A., et al. (U.S. Pat. No. 5,676,950, issued Oct. 14, 1997, herein incorporated by reference).

Other viral vectors include those derived from the pox family of viruses, including vaccinia virus and avian poxvirus. By way of example, vaccinia virus recombinants expressing a nucleic acid molecule of interest (e.g., engineered retron) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

In some embodiments, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., engineered retron) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

In other approaches to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2nd edition, 2007) and L. King The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant cells. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

To obtain expression of the engineered retron or the ncRNA encoded thereby, the expression construct or the ncRNA must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

Several non-viral methods for the transfer of expression constructs into cultured cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

Once the expression construct has been delivered into the cell the nucleic acid comprising the engineered retron sequence may be positioned and expressed at different sites. In some embodiments, the nucleic acid comprising the engineered retron sequence may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or episomes encode sequences sufficient to permit maintenance and replication independent of or in synchronization with the host cell cycle. How the expression construct is delivered to a cell and where in the cell the nucleic acid remains is dependent on the type of expression construct employed.

In some embodiments, the expression construct may simply consist of naked recombinant DNA or plasmids comprising the engineered retron. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding an engineered retron of interest may also be transferred in a similar manner in vivo and express retron products.

In still another embodiment, a naked DNA expression construct may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

In a further embodiment, the expression construct may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In some embodiments, the liposome may be complexed with a hemagglutinating virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364).

In yet further embodiments, the liposome may be complexed or employed in conjunction with both HVJ and HMG-I. Where a bacterial promoter is employed in the DNA construct, it also will be desirable to include within the liposome an appropriate bacterial polymerase.

Other expression constructs which can be employed to deliver a nucleic acid into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167). Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (Methods Enzymol. (1987) 149:157-176) employed lactosy 1-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In some embodiments, the promoters that may be used in the retron delivery systems described herein may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-b promoter, Mb promoter, Nphsl promoter, OG-2 promoter, SP-B promoter, SYN1 promoter, and WASP promoter.

### 1. Delivery Systems and Methods of Delivery

### Overview

In yet another aspect, the disclosure provides vectors for transferring and/or expressing said retron-based gene editing systems, e.g., under in vitro, ex vivo, and in vivo conditions. In still another aspect, the disclosure provides cell-delivery compositions and methods, including compositions for passive and/or active transport to cells (e.g., plasmids), delivery by virus-based recombinant vectors (e.g., AAV and/or lentivirus vectors), delivery by non-virus-based systems (e.g., liposomes and LNPs), and delivery by virus-like particles of the retron-based gene editing systems described herein. Depending on the delivery system employed, the retron-based gene editing systems described herein may be delivered in the form of DNA (e.g., plasmids or DNA-based virus vectors), RNA (e.g., guide RNA and mRNA delivered by LNPs), a mixture of DNA and RNA, protein (e.g., virus-like particles), and ribonucleoprotein (RNP) complexes. Any suitable combinations of approaches for delivering the components of the herein disclosed retron-based gene editing systems may be employed.

The retron-based gene editing systems and/or components thereof can be delivered by any known delivery system such as those described above, including (a) without vectors (e.g., electroporation), (b) viral delivery systems and (c) non-viral delivery systems. Viral delivery systems include expression vectors, adeno-associated virus (AAV) vectors, retroviral vectors, lentiviral vectors, and the like. An expression construct can be replicated in a living cell, or it can be made synthetically. Non-viral delivery systems include without limitation lipid particles (e.g. Lipid nanoparticles (LNPs)), non-lipid nanoparticles, exosomes, liposomes, micelles, viral particles, stable nucleic-acid-lipid particles (SNALPs), lipoplexes/polyplexes, DNA nanoclews, Gold nanoparticles, iTOP, Streptolysin O (SLO), multifunctional envelope-type nanodevice (MEND), lipid-coated mesoporous silica particles, inorganic nanoparticles, and polymeric delivery technology (e.g., polymer-based particles).

Delivery of nucleic acid modalities, including RNA therapeutics, is described further in Paunovska K, Loughrey D, Dahlman J E. Drug delivery systems for RNA therapeutics. Nat Rev Genet. 2022 May; 23(5):265-280. doi: 10.1038/s41576-021-00439-4. Epub 2022 Jan. 4. PMID: 34983972; PMCID: PMC8724758; Hong C A, Nam Y S. Functional nanostructures for effective delivery of small interfering RNA therapeutics. Theranostics. 2014 Sep. 19; 4(12):1211-32. doi: 10.7150/thno.8491. PMID: 25285170; PMCID: PMC4183999; Liu F, Wang C, Gao Y, Li X, Tian F, Zhang Y, Fu M, Li P, Wang Y, Wang F. Current Transport Systems and Clinical Applications for Small Interfering RNA (siRNA) Drugs. Mol Diagn Ther. 2018 October; 22(5):551-569. doi: 10.1007/s40291-018-0338-8. PMID: 29926308; Zhang Y, Almazi J G, Ong H X, Johansen M D, Ledger S, Traini D, Hansbro P M, Kelleher A D, Ahlenstiel C L. Nanoparticle Delivery Platforms for RNAi Therapeutics Targeting COVID-19 Disease in the Respiratory Tract. Int J Mol Sci. 2022 Feb. 22; 23(5):2408. doi: 10.3390/ijms23052408. PMID: 35269550; PMCID: PMC8909959; Zhang M, Hu S, Liu L, Dang P, Liu Y, Sun Z, Qiao B, Wang C. Engineered exosomes from different sources for cancer-targeted therapy. Signal Transduct Target Ther. 2023 Mar. 15; 8(1):124. doi: 10.1038/s41392-023-01382-y. PMID: 36922504; PMCID: PMC10017761; Hastings M L, Krainer A R. RNA therapeutics. RNA. 2023 April; 29(4):393-395. doi: 10.1261/rna.079626.123. PMID: 36928165; PMCID: PMC10019368; Miele E, Spinelli G P, Miele E, Di Fabrizio E, Ferretti E, Tomao S, Gulino A. Nanoparticle-based delivery of small interfering RNA: challenges for cancer therapy. Int J Nanomedicine. 2012; 7:3637-57. doi: 10.2147/IJN.S23696. Epub 2012 Jul. 20. PMID: 22915840; PMCID: PMC3418108, each of which are incorporated by reference in their entireties.

The engineered retron-based gene editing systems (or vectors containing them) may be introduced into any type of cell, including any cell from a prokaryotic, eukaryotic, or archaeon organism, including bacteria, archaea, fungi, protists, plants (e.g., monocotyledonous and dicotyledonous plants); and animals (e.g., vertebrates and invertebrates). Examples of animals that may be transfected with an engineered retron-based gene editing systems include, without limitation, vertebrates such as fish, birds, mammals (e.g., human and non-human primates, farm animals, pets, and laboratory animals), reptiles, and amphibians.

The engineered retron-based gene editing systems (or components thereof) can be introduced into a single cell or a population of cells. Cells from tissues, organs, and biopsies, as well as recombinant cells, genetically modified cells, cells from cell lines cultured in vitro, and artificial cells (e.g., nanoparticles, liposomes, polymersomes, or microcapsules encapsulating nucleic acids) may all be transfected with the engineered retron-based gene editing systems.

The engineered retron-based gene editing systems (or components thereof) can be introduced into cellular fragments, cell components, or organelles (e.g., mitochondria in animal and plant cells, plastids (e.g., chloroplasts) in plant cells and algae).

Cells may be cultured or expanded after transfection with the engineered retron-based gene editing systems.

Methods of introducing nucleic acids into a host cell are well known in the art. Commonly used methods include chemically induced transformation, typically using divalent cations (e.g., CaCl₂), dextran-mediated transfection, polybrene mediated transfection, lipofectamine and LT-1 mediated transfection, electroporation, protoplast fusion, encapsulation of nucleic acids in liposomes, and direct microinjection of the nucleic acids comprising Cas12a editing systems into nuclei. See, e.g., Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197; herein incorporated by reference in their entireties.

Plant cells may also be targeted by the retron-based gene editing systems (or components thereof) disclosed herein. Methods for genetic transformation of plant cells are known in the art and include those set forth in US2022/0145296, and U.S. Pat. Nos. 8,575,425; 7,692,068; 8,802,934; 7,541,517; each of which is herein incorporated by reference in its entirety. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G. W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; and Jones et al. (2005) Plant Methods 1:5.

The plant cells that have been transformed may be grown into a transgenic organism, such as a plant, in accordance with conventional methods. See, for example, McCormick et al. (1986) Plant Cell Reports 5:81-84.

Plant material that may be transformed with the retron-based gene editing systems (or components thereof) described herein includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Progeny, variants, and mutants of the regenerated plants are also included within the scope of the disclosure, provided that these parts comprise the genetic modification introduced by the retron-based gene editing systems. Further provided is a processed plant product or byproduct that retains the genetic modification introduced by the retron-based gene editing systems.

The retron-based gene editing systems described herein may be used to produce transgenic plants with desired phenotypes, including but not limited to, increased disease resistance (e.g., increased viral, bacterial of fungal resistance), increased insect resistance, increased drought resistance, increased yield, and altered fruit ripening characteristics, sugar and oil composition, and color.

In some embodiments involving retron-based gene editing systems, the retron msr gene, msd gene, and/or ret gene can be expressed in vitro from a vector, such as in an in vitro transcription system. The resulting ncRNA or msDNA can be isolated before being packaged and/or formulated for direct delivery into a host cell. For example, the isolated ncRNA or msDNA can be packaged/formulated in a delivery vehicle such as lipid nanoparticles as described in other sections.

In some embodiments involving retron-based gene editing systems, the retron msr gene, msd gene, and/or ret gene are expressed in vivo from a vector within a cell. The retron msr gene, msd gene, and/or ret gene can be introduced into a cell with a single vector or in multiple separate vectors to produce msDNA in a host subject.

In other embodiments, the retron msr gene, msd gene, and/or ret gene, and any other components of the retron-based genome editing systems described herein (e.g., guide RNA in trans, programmable nuclease (e.g., in trans)) may be expressed in vivo from RNA delivered to the cell. The retron msr gene, msd gene, and/or ret gene can be introduced into a cell with a single vector or in multiple separate vectors to produce msDNA in a host subject.

Vectors and/or nucleic acid molecules encoding the recombinant retron-based genome editing system or components thereof can include control elements operably linked to the retron sequences, which allow for the production of msDNA either in vitro, or in vivo in the subject species. For example, in embodiments relating to retron-based gene editing systems, the retron msr gene, msd gene, and/or ret gene can be operably linked to a promoter to allow expression of the retron reverse transcriptase and/or the msDNA product. In some embodiments, heterologous sequences encoding desired products of interest (e.g., polynucleotide encoding polypeptide or regulatory RNA, donor polynucleotide for gene editing, or protospacer DNA for molecular recording) may be inserted in the msr gene and/or msd gene.

In some embodiments, the retron-based gene editing systems are produced by a vector system comprising one or more vectors.

Numerous vectors are available for use in the vector or vector system, including but not limited to, linear polynucleotides, polynucleotides associated with ionic or amphiphilic compounds, plasmids, and viruses.

### All-RNA Format

In various embodiments, the retron-based gene editing systems (or components thereof) disclosed herein may be delivered in an “all-RNA” format. As used herein, the term “all-RNA” format refers to the fact that each of the components of a retron editing system (e.g., the retron RT, the programmable nuclease, the sgRNA, and the ncRNA) are delivered and/or administered as RNA (e.g., coding RNA or non-coding RNA). In some embodiments, the RNA components may be delivered to cells and/or tissues by direct means, such as electroporation or transfection. In other embodiments, the RNA components may be delivered to cells and/or tissues by way of a delivery vehicle, such as an LNP or liposome.

In various embodiments, the retron editing systems described herein may comprise a coding RNA (e.g., linear or circular mRNA) that encodes a retron reverse transcriptase (e.g., any RT from Table X or Table A), a coding RNA (e.g., linear or circular mRNA) that encodes a programmable nuclease (e.g., a Cas9, Cas12a, or TnpB nuclease), a retron ncRNA (e.g., a ncRNA from Table B), and a guide RNA for targeting the programmable nuclease to a particular desired target sequence.

In some embodiments, RT and nuclease components may be encoded on the same coding RNA molecule. The proteins may also be expressed from separate coding RNA molecules. In still other embodiments, the RT and the nuclease components can be fused together as a singular fusion polypeptide having an RT domain and a nuclease domain optionally joined by a linker.

In addition, in some embodiments, the ncRNA and the guide RNA may be fused together as a single RNA molecule. For example, the guide RNA may be located at the 5′ end of the ncRNA. In other embodiments, the guide RNA may be located at the 3′ end of the ncRNA. In some embodiments, the ncRNA may comprise a guide RNA at both the 3′ and the 5′ ends of the ncRNA.

In still other embodiments, the ncRNA and the guide RNA may be separate molecules, i.e., delivered separately.

In still other embodiments, the retron editing system may include both a ncRNA-guide RNA fusion and an additional guide RNA provided as a separate molecule.

In various embodiments, the different RNA components of the all-RNA retron editing system can be combined and administered (e.g., directly or within a delivery vehicle) in different ratios. In some embodiments, the ratios of such RNA components or species can be expressed as molar ratios.

For example, the molar ratio of RT coding RNA to nuclease coding RNA can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In another example, the molar ratio of nuclease coding RNA to RT coding RNA can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of ncRNA or ncRNA-guide RNA fusion to separate guide RNA can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of separate guide RNA to ncRNA or ncRNA-guide RNA fusion can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of ncRNA to separate guide RNA can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of separate guide RNA to ncRNA can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of a coding RNA (e.g., encoding RT and/or nuclease) to ncRNA or ncRNA-guide RNA fusion, as the case may be, can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of a coding RNA encoding a retron RT to ncRNA or ncRNA-guide RNA fusion, as the case may be, can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of a coding RNA encoding a programmable nuclease to ncRNA or ncRNA-guide RNA fusion, as the case may be, can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of a coding RNA encoding a retron RT or a nuclease to a separate guide RNA can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In still another example, the molar ratio of a separate guide RNA to a coding RNA encoding a retron RT or a nuclease can be about 1:1, about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40.

In certain embodiments, the amount of ncRNA-sgRNA relative to RT mRNA is augmented. In certain embodiments the RT mRNA: ncRNA-sgRNA ratio is about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40. In certain embodiments, an RT-Cas9 (or Cas9-RT) fusion is encoded by an mRNA. In certain embodiments, the RT-Cas9 mRNA: ncRNA-sgRNA ratio is about 1:1.5, about 1:2, about 1:2.5, about 1:3, about 1:4, about 1:5, about 1:6, about 1:7, about 1:8, about 1:9, about 1:10, about 1:12, about 1:15, about 1:20. Useful ranges include from 1:1 to 1:2, from 1:1.5 to 1:4, from 1:2 to 1:4, from 1:2 to 1:8, from 1:2 to 1:10, from 1:3 to 1:9, from 1:3 to 1:12, from 1:3 to 1:15, from 1:4 to 1:8, from 1:4 to 1:12, from 1:4 to 1:20, from 1:5 to 1:10, from 1:5 to 1:15, from 1:5 to 1:20, from 1:10 to 1:20, or from 1:10 to 1:40. In certain embodiments, multiple genetic loci are targeted hence the ncRNA-sgRNA includes a mixture of ncRNA-sgRNA species and the same ratios and ranges are applicable.

### Viral Vector Delivery

In various embodiments, the retron-based gene editing systems described herein may be delivered in viral vectors.

Examples of viral vectors include, but are not limited to, adenoviral vectors, adeno-associated virus (AAV) vectors, retroviral vectors, lentiviral vectors, and the like. An expression construct can be replicated in a living cell, or it can be made synthetically.

In some embodiments, the nucleic acid comprising an retron-based gene editing system (or component thereof) is under transcriptional control of a promoter. In some embodiments, the promoter is competent for initiating transcription of an operably linked coding sequence by a RNA polymerase I, II, or III.

Exemplary promoters for mammalian cell expression include the SV40 early promoter, a CMV promoter such as the CMV immediate early promoter (see, U.S. Pat. Nos. 5,168,062 and 5,385,839, incorporated herein by reference in their entireties), the mouse mammary tumor virus LTR promoter, the adenovirus major late promoter (Ad MLP), and the herpes simplex virus promoter, among others. Other nonviral promoters, such as a promoter derived from the murine metallothionein gene, will also find use for mammalian expression.

Exemplary promoters for plant cell expression include the CaMV 35S promoter (Odell et al., 1985, Nature 313:810-812); the rice actin promoter (McElroy et al., 1990, Plant Cell 2:163-171); the ubiquitin promoter (Christensen et al., 1989, Plant Mol. Biol. 12:619-632; and Christensen et al., 1992, Plant Mol. Biol. 18:675-689); the pEMU promoter (Last et al., 1991, Theor. Appl. Genet. 81:581-588); and the MAS promoter (Velten et al., 1984, EMBO J. 3:2723-2730).

In additional embodiments, the retron-based vectors may also comprise tissue-specific promoters to start expression only after it is delivered into a specific tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-b promoter, Mb promoter, Nphsl promoter, OG-2 promoter, SP-B promoter, SYN1 promoter, and WASP promoter.

These and other promoters can be obtained from or incorporated into commercially available plasmids, using techniques well known in the art. See, e.g., Sambrook et al., supra.

In some embodiments, one or more enhancer elements is/are used in association with the promoter to increase expression levels of the constructs. Examples include the SV40 early gene enhancer, as described in Dijkema et al., EMBOJ (1985) 4:761, the enhancer/promoter derived from the long terminal repeat (LTR) of the Rous Sarcoma Virus, as described in Gorman et al., Proc. Natl. Acad. Sci. USA (1982b) 79:6777, and elements derived from human CMV, as described in Boshart et al., Cell (1985) 41:521, such as elements included in the CMV intron A sequence. All such sequences are incorporated herein by reference.

In one embodiment, an expression vector for expressing a retron-based gene editing system (or component thereof) comprises a promoter operably linked to a polynucleotide encoding the components. The components of the retron-based gene editing system may be configured as individual gene transcripts or as fused constructs. For example, the nuclease component may be fused with the reverse transcriptase component. In another example, the ncRNA component may be fused with the guide RNA component. In another example, the nuclease component may be fused with the reverse transcriptase component, but the guide RNA and the ncRNA are separate. In other embodiments, the guide RNA and ncRNA components may be fused, but the reverse transcriptase and nuclease components are separately provided. Any functional combinations of fused components and non-fused components are contemplated.

In some embodiments, the vector or vector system also comprises a transcription terminator/polyadenylation signal. Examples of such sequences include, but are not limited to, those derived from SV40, as described in Sambrook et al., supra, as well as a bovine growth hormone terminator sequence (see, e.g., U.S. Pat. No. 5,122,458).

Additionally, 5′-UTR sequences can be placed adjacent to the coding sequence to further enhance the expression. Such sequences may include UTRs comprising an internal ribosome entry site (IRES). Inclusion of an IRES permits the translation of one or more open reading frames from a vector. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298: Rees et al., BioTechniques (1996) 20:102-110; Kobayashi et al., BioTechniques (1996) 21:399-402; and Mosser et al., BioTechniques (199722 ISO-161)c. A multitude of IRES sequences are known and include sequences derived from a wide variety of viruses, such as from leader sequences of picomaviruses such as the encephalomyocarditis virus (EMCV) UTR (Jang et al. Virol. (1989) 63:1651-1660). the polio leader sequence, the hepatitis A virus leader, the hepatitis C virus IRES, human rhinovirus type 2 IRES (Dobrikova et al., Proc. Natl. Acad. Sci. (2003) 100(251:15125-151301)). an IRES element from the foot and mouth disease virus (Ramesh et al., Nucl. Acid Res. (1996) 24:2697-2700), a giardiavirus IRES (Garlapati et al., J Biol. Chem. (2004) 279(51):3389-33971) and the like. A variety of nonviral IRES sequences will also find use herein, including, but not limited to IRES sequences from yeast, as well as the human angiotensin II type 1 receptor IRES (Martin et al., Mol. Cell Endocrinol. (2003) 212:51-61), fibroblast growth factor IRESs (FGF-1 IRES and FGF-2 IRES, Martineau et al. (2004) Mol. Cell. Biol. 24(17): 7622-7635), vascular endothelial growth factor IRES (Baranick et al. (2008) Proc. Natl. Acad Sci. U.S.A. 105(12):4733-4738, Stein et al. (1998) Mol. Cell. Biol. 18(6):3112-3119, Bert et al. (2006) RNA 12(6): 1074-1083), and insulin-like growth factor 2 IRES (Pedersen et al. (2002) Biochem. J. 363(Pt 1):37-44).

These elements are commercially available in plasmids sold, e.g., by Clontech (Mountain View, CA), Invivogen (San Diego, CA), Addgene (Cambridge, MA) and GeneCopoeia (Rockville, MD). See also IRESite: The database of experimentally verified IRES structures (iresite.org). An IRES sequence may be included in a vector, for example, to express multiple bacteriophage recombination proteins for recombineering or an RNA-guided nuclease (e.g., Cas9) for HDR in combination with a retron reverse transcriptase from an expression cassette.

In some embodiments, a polynucleotide encoding a viral self-cleaving 2A peptide, such as a T2A peptide, can be used to allow production of multiple protein products (e.g., Cas9, bacteriophage recombination proteins, retron reverse transcriptase) from a single vector or a single transcription unit under one promoter. One or more 2A linker peptides can be inserted between the coding sequences in the multicistronic construct. The 2A peptide, which is self-cleaving, allows co-expressed proteins from the multicistronic construct to be produced at equimolar levels. 2A peptides from various viruses may be used, including, but not limited to 2A peptides derived from the foot-and-mouth disease virus, equine rhinitis A virus, Jhosea asigna virus and porcine teschovirus-1. See, e.g., Kim et al. (2011) PLoS One 6(4): e18556, Trichas et al. (2008) BMC Biol. 6:40, Provost et al. (2007) Genesis 45(10): 625-629, Furler et al. (2001) Gene Ther. 8(11):864-873; herein incorporated by reference in their entireties.

In some embodiments, the expression construct comprises a plasmid suitable for transforming a bacterial host. Numerous bacterial expression vectors are known to those of skill in the art, and the selection of an appropriate vector is a matter of choice. Bacterial expression vectors include, but are not limited to, pACYC177, pASK75, pBAD, pBADM, pBAT, pCal, pET, pETM, pGAT, pGEX, pHAT, pKK223, pMal, pProEx, pQE, and pZA31 Bacterial plasmids may contain antibiotic selection markers (e.g., ampicillin, kanamycin, erythromycin, carbenicillin, streptomycin, or tetracycline resistance), a lacZ gene (b-galactosidase produces blue pigment from x-gal substrate), fluorescent markers (e.g., GFP. mCherry), or other markers for selection of transformed bacteria. See, e.g., Sambrook et al., supra.

In other embodiments, the expression construct comprises a plasmid suitable for transforming a yeast cell. Yeast expression plasmids typically contain a yeast-specific origin of replication (ORI) and nutritional selection markers (e.g., HIS3, URA3, LYS2, LEU2, TRP1, METIS, ura4+, leul+, ade6+), antibiotic selection markers (e.g., kanamycin resistance), fluorescent markers (e.g., mCherry), or other markers for selection of transformed yeast cells. The yeast plasmid may further contain components to allow shuttling between a bacterial host (e.g., E coif) and yeast cells. A number of different types of yeast plasmids are available including yeast integrating plasmids (Yip), which lack an ORI and are integrated into host chromosomes by homologous recombination; yeast replicating plasmids (YRp), which contain an autonomously replicating sequence (ARS) and can replicate independently; yeast centromere plasmids (YCp), which are low copy vectors containing a part of an ARS and part of a centromere sequence (CEN); and yeast episomal plasmids (YEp), which are high copy number plasmids comprising a fragment from a 2 micron circle (a natural yeast plasmid) that allows for 50 or more copies to be stably propagated per cell.

In other embodiments, the expression construct does not comprise a plasmid suitable for transforming a yeast cell.

In other embodiments, the expression construct comprises a virus or engineered construct derived from a viral genome. A number of viral based systems have been developed for gene transfer into mammalian cells. These include adenoviruses, retroviruses (g-retroviruses and lentiviruses), poxviruses, adeno-associated viruses, baculoviruses, and herpes simplex viruses (see e.g., Wamock et al. (2011) Methods Mol. Biol. 737:1-25; Walther et al. (2000) Drugs 60(2):249-271; and Lundstrom (2003) Trends Biotechnol. 21(3): 117-122; herein incorporated by reference in their entireties). The ability of certain viruses to enter cells via receptor-mediated endocytosis, to integrate into host cell genomes and express viral genes stably and efficiently have made them attractive candidates for the transfer of foreign genes into mammalian cells.

For example, retroviruses provide a convenient platform for gene delivery systems. Selected sequences can be inserted into a vector and packaged in retroviral particles using techniques known in the art. The recombinant virus can then be isolated and delivered to cells of the subject either in vivo or ex vivo. A number of retroviral systems have been described (U.S. Pat. No. 5,219,740; Miller and Rosman (1989) BioTechniques 7:980-990; Miller, A. D. (1990) Human Gene Therapy 1:5-14; Scarpa et al. (1991) Virology 180:849-852; Bums et al. (1993) Proc. Natl. Acad. Sci. USA 90:8033-8037; Boris-Lawrie and Temin (1993) Cur. Opin. Genet. Develop. 3:102-109; and Ferry et al. (2011) Curr. Pharm. Des. 17(24): 2516-2527). Lentiviruses are a class of retroviruses that are particularly useful for delivering polynucleotides to mammalian cells because they are able to infect both dividing and nondividing cells (see e.g., Lois et al. (2002) Science 295:868-872; Durand et al. (2011) Viruses 3(2): 132-159; herein incorporated by reference).

A number of adenoviral vectors have also been described. Unlike retroviruses which integrate into the host genome, adenoviruses persist extrachromosomally thus minimizing the risks associated with insertional mutagenesis.

Additionally, various adeno-associated vims (AAV) vector systems have been developed for gene delivery. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al., Molec. Cell. Biol. (1988) 8:3988-3996; Vincent et al., Vaccines 90 (1990) (Cold Spring Harbor LaboratoryPress); Carter, B. J. Current Opinion in Biotechnology (1992) 3:533-539; Muzyczka, N. Current Topics in Microbiol and Immunol. (1992) 158:97-129; Kotin, R. M. Human Gene Therapy (1994) 5:793-801; Shelling and Smith, Gene Therapy (1994) 1:165-169; and Zhou et al., J. Exp. Med. (1994) 179:1867-1875.

Another vector system useful for delivering nucleic acids encoding the Cas12a editing system components is the enterically administered recombinant poxvirus vaccines described by Small, Jr., P. A., et al. (U.S. Pat. No. 5,676,950, issued Oct. 14, 1997, herein incorporated by reference).

Other viral vectors include those derived from the pox family of viruses, including vaccinia virus and avian poxvirus. By way of example, vaccinia virus recombinants expressing a nucleic acid molecule of interest (e.g., Cas12a editing system) can be constructed as follows. The DNA encoding the particular nucleic acid sequence is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the sequences of interest into the viral genome. The resulting TK-recombinant can be selected by culturing the cells in the presence of 5-bromodeoxyuridine and picking viral plaques resistant thereto.

In some embodiments, avipoxviruses, such as the fowlpox and canarypox viruses, can also be used to deliver the nucleic acid molecules of interest. The use of an avipox vector is particularly desirable in human and other mammalian species since members of the avipox genus can only productively replicate in susceptible avian species and therefore are not infective in mammalian cells. Methods for producing recombinant avipoxviruses are known in the art and employ genetic recombination, as described above with respect to the production of vaccinia viruses. See, e.g., WO 91/12882; WO 89/03429; and WO 92/03545.

Molecular conjugate vectors, such as the adenovirus chimeric vectors described in Michael et al., J. Biol. Chem. (1993) 268:6866-6869 and Wagner et al., Proc. Natl. Acad. Sci. USA (1992) 89:6099-6103, can also be used for gene delivery.

Members of the alphavirus genus, such as, but not limited to, vectors derived from the Sindbis virus (SIN), Semliki Forest virus (SFV), and Venezuelan Equine Encephalitis virus (VEE), will also find use as viral vectors for delivering the polynucleotides of the present invention. For a description of Sindbis-virus derived vectors useful for the practice of the instant methods, see, Dubensky et al. (1996) J. Virol. 70:508-519; and International Publication Nos. WO 95/07995, WO 96/17072; as well as, Dubensky, Jr., T. W., et al., U.S. Pat. No. 5,843,723, issued Dec. 1, 1998, and Dubensky, Jr., T. W., U.S. Pat. No. 5,789,245, issued Aug. 4, 1998, both herein incorporated by reference. Particularly preferred are chimeric alphavirus vectors comprised of sequences derived from Sindbis virus and Venezuelan equine encephalitis virus. See, e.g., Perri et al. (2003) J. Virol. 77: 10394-10403 and International Publication Nos. WO 02/099035, WO 02/080982, WO 01/81609, and WO 00/61772; herein incorporated by reference in their entireties.

A vaccinia-based infection/transfection system can be conveniently used to provide for inducible, transient expression of the nucleic acids of interest (e.g., Cas12a editing system) in a host cell. In this system, cells are first infected in vitro with a vaccinia virus recombinant that encodes the bacteriophage T7 RNA polymerase. This polymerase displays exquisite specificity in that it only transcribes templates bearing T7 promoters. Following infection, cells are transfected with the nucleic acid of interest, driven by a T7 promoter. The polymerase expressed in the cytoplasm from the vaccinia virus recombinant transcribes the transfected DNA into RNA. The method provides for high level, transient, cytoplasmic production of large quantities of RNA. See, e.g., Elroy-Stein and Moss, Proc. Natl. Acad. Sci. USA (1990) 87:6743-6747; Fuerst et al., Proc. Natl. Acad. Sci. USA (1986) 83:8122-8126.

In other approaches to infection with vaccinia or avipox virus recombinants, or to the delivery of nucleic acids using other viral vectors, an amplification system can be used that will lead to high level expression following introduction into host cells. Specifically, a T7 RNA polymerase promoter preceding the coding region for T7 RNA polymerase can be engineered. Translation of RNA derived from this template will generate T7 RNA polymerase which in turn will transcribe more templates. Concomitantly, there will be a cDNA whose expression is under the control of the T7 promoter. Thus, some of the T7 RNA polymerase generated from translation of the amplification template RNA will lead to transcription of the desired gene. Because some T7 RNA polymerase is required to initiate the amplification, T7 RNA polymerase can be introduced into cells along with the template(s) to prime the transcription reaction. The polymerase can be introduced as a protein or on a plasmid encoding the RNA polymerase. For a further discussion of T7 systems and their use for transforming cells, see, e.g., International Publication No. WO 94/26911; Studier and Moffatt, J. Mol. Biol. (1986) 189:113-130; Deng and Wolff, Gene (1994) 143:245-249; Gao et al., Biochem. Biophys. Res. Commun. (1994) 200:1201-1206; Gao and Huang, Nuc. Acids Res. (1993) 21:2867-2872; Chen et al., Nuc. Acids Res. (1994) 22:2114-2120; and U.S. Pat. No. 5,135,855.

Insect cell expression systems, such as baculovirus systems, can also be used and are known to those of skill in the art and described in, e.g., Baculovirus and Insect Cell Expression Protocols (Methods in Molecular Biology, D. W. Murhammer ed., Humana Press, 2nd edition, 2007) and L. King The Baculovirus Expression System: A laboratory guide (Springer, 1992). Materials and methods for baculovirus/insect cell expression systems are commercially available in kit form from, inter alia, Thermo Fisher Scientific (Waltham, MA) and Clontech (Mountain View, CA).

Plant expression systems can also be used for transforming plant cells. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

To obtain expression of the retron-based editing systems (or components thereof), the expression constructs and/or RNA components must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsulated in an infectious viral particle.

### Non-Viral Delivery Methods

Several non-viral methods for the transfer of expression constructs are available for delivering the retron-based editing systems or components thereof into cells also are contemplated. These include the use of calcium phosphate precipitation, DEAE-dextran, electroporation, direct microinjection, DNA-loaded liposomes, lipofectamine-DNA complexes, cell sonication, gene bombardment using high velocity microprojectiles, and receptor-mediated transfection (see, e.g., Graham and Van Der Eb (1973) Virology 52:456-467; Chen and Okayama (1987) Mol. Cell Biol. 7:2745-2752; Rippe et al. (1990) Mol. Cell Biol. 10:689-695; Gopal (1985) Mol. Cell Biol. 5:1188-1190; Tur-Kaspa et al. (1986) Mol. Cell. Biol. 6:716-718; Potter et al. (1984) Proc. Natl. Acad. Sci. USA 81:7161-7165); Harland and Weintraub (1985) J. Cell Biol. 101:1094-1099); Nicolau & Sene (1982) Biochim. Biophys. Acta 721:185-190; Fraley et al. (1979) Proc. Natl. Acad. Sci. USA 76:3348-3352; Fechheimer et al. (1987) Proc Natl. Acad. Sci. USA 84:8463-8467; Yang et al. (1990) Proc. Natl. Acad. Sci. USA 87:9568-9572; Wu and Wu (1987) J. Biol. Chem. 262:4429-4432; Wu and Wu (1988) Biochemistry 27:887-892; herein incorporated by reference). Some of these techniques may be successfully adapted for in vivo or ex vivo use.

In some embodiments, nucleic acid molecules encoding the retron-based editing systems or components thereof may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or episomes encode sequences sufficient to permit maintenance and replication independent of or in synchronization with the host cell cycle. How the expression construct is delivered to a cell and where in the cell the nucleic acid remains is dependent on the type of expression construct employed.

In some embodiments, expression constructs encoding the retron-based editing systems or components thereof may simply consist of naked recombinant DNA or plasmids comprising nucleotide sequences encoding said retron-based editing systems or components thereof. Transfer of the construct may be performed by any of the methods mentioned above which physically or chemically permeabilize the cell membrane. This is particularly applicable for transfer in vitro but it may be applied to in vivo use as well. Dubensky et al. (Proc. Natl. Acad. Sci. USA (1984) 81:7529-7533) successfully injected polyomavirus DNA in the form of calcium phosphate precipitates into liver and spleen of adult and newborn mice demonstrating active viral replication and acute infection. Benvenisty & Neshif (Proc. Natl. Acad. Sci. USA (1986) 83:9551-9555) also demonstrated that direct intraperitoneal injection of calcium phosphate-precipitated plasmids results in expression of the transfected genes. It is envisioned that DNA encoding a retron-based editing system or components thereof of interest may also be transferred in a similar manner in vivo and express retron products.

In still another embodiment, DNA expression constructs encoding the retron-based editing systems or components thereof may be transferred into cells by particle bombardment. This method depends on the ability to accelerate DNA-coated microprojectiles to a high velocity allowing them to pierce cell membranes and enter cells without killing them (Klein et al. (1987) Nature 327:70-73). Several devices for accelerating small particles have been developed. One such device relies on a high voltage discharge to generate an electrical current, which in turn provides the motive force (Yang et al. (1990) Proc. Natl. Acad. Sci. USA 87:9568-9572). The microprojectiles may consist of biologically inert substances, such as tungsten or gold beads.

### Liposomes

In a further embodiment, constructs encoding the retron-based editing systems or components thereof may be delivered using liposomes. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh & Bachhawat (1991) Liver Diseases, Targeted Diagnosis and Therapy Using Specific Receptors and Ligands, Wu et al. (Eds.), Marcel Dekker, NY, 87-104). Also contemplated is the use of lipofectamine-DNA complexes.

In some embodiments, the liposome may be complexed with a hemagglutinating virus (HVJ). This has been shown to facilitate fusion with the cell membrane and promote cell entry of liposome-encapsulated DNA (Kaneda et al. (1989) Science 243:375-378). In other embodiments, the liposome may be complexed or employed in conjunction with nuclear non-histone chromosomal proteins (HMG-I) (Kato et al. (1991) J. Biol. Chem. 266(6):3361-3364).

In yet further embodiments, the liposome may be complexed or employed in conjunction with both HVJ and HMG-I. Where a bacterial promoter is employed in the DNA construct, it also will be desirable to include within the liposome an appropriate bacterial polymerase.

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In one embodiment, the liposome comprises a transport polymer, which may optionally be branched, comprising at least 10 amino acids and a ratio of histidine to non-histidine amino acids greater than 1.5 and less than 10. The branched transport polymer can comprise one or more backbones, one or more terminal branches, and optionally, one or more non-terminal branches. See, U.S. Pat. No. 7,070,807, incorporated herein by reference in its entirety. In one embodiment, the transport polymer is a Histidine-Lysine co-polymer (HKP) used to package and deliver mRNA and other cargos. See, U.S. Pat. Nos. 7,163,695, and 7,772,201, incorporated herein by reference in their entireties,

In one embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-eDMA, and 1,2-dilinoleyloxy-3-(N;N-dimethyl)aminopropane (DLinDMA).

### Polymer Based Vehicles

In one embodiment, the retron-based editing systems or components thereof may be encapsulated by delivery vehicles that comprise polymer-based particles (e.g., nanoparticles). In one embodiment, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or snucleic acid component, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles are VIROMER, e.g., VIROMER RNAi, VIROMER RED, VIROMER mRNA. Example methods of delivering the systems and compositions herein include those described in Bawage S S et al., Synthetic mRNA expressed Cast 3a mitigates RNA virus infections, biorxiv.org/content/10.1101/370460v1. full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi: 10.13140/RG.2.2.23912.16642.

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; El-Andaloussi S, et al., Nat Protoc. 2012 December; 7(12):2112-26; Uno Y, et al., Hum

Gene Ther. 2011 June; 22(6):711-9; Zou W, et al., Hum Gene Then 2011 April; 22(4):465-75. Exemplary exosomes can be generated from 293F cells, with mRNA-loaded exosomes driving higher mRNA expression than mRNA loaded LNPs in some instances. See, e.g. J. Biol. Chem. (2021) 297(5) 101266

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/dObm00427h.

### Receptor-Mediated Delivery

Other expression constructs encoding the retron-based editing systems or components thereof are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu (1993) Adv. Drug Delivery Rev. 12:159-167). Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) and transferrin (see, e.g., Wu and Wu (1987), supra; Wagner et al. (1990) Proc. Natl. Acad. Sci. USA 87(9):3410-3414). A synthetic neoglycoprotein, which recognizes the same receptor as ASOR, has been used as a gene delivery vehicle (Ferkol et al. (1993) FASEB J. 7:1081-1091; Perales et al. (1994) Proc. Natl. Acad. Sci. USA 91(9):4086-4090), and epidermal growth factor (EGF) has also been used to deliver genes to squamous carcinoma cells (Myers, EPO 0273085).

In other embodiments, delivery vehicle comprising one or more expression constructs encoding the retron-based editing systems or components thereof may comprise a ligand and a liposome. For example, Nicolau et al. (Methods Enzymol. (1987) 149:157-176) employed lactosy 1-ceramide, a galactose-terminal asialoganglioside, incorporated into liposomes and observed an increase in the uptake of the insulin gene by hepatocytes. Thus, it is feasible that a nucleic acid encoding a particular gene also may be specifically delivered into a cell by any number of receptor-ligand systems with or without liposomes. Also, antibodies to surface antigens on cells can similarly be used as targeting moieties.

In some embodiments, the promoters that may be used in the retron-based editing systems or components thereof described herein may be constitutive, inducible, or tissue-specific. In some embodiments, the promoters may be a constitutive promoters. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EFla) promoter, ubiquitin promoters, actin promoters, tubulin promoters, immunoglobulin promoters, a functional fragment thereof, or a combination of any of the foregoing. In some embodiments, the promoter may be a CMV promoter. In some embodiments, the promoter may be a truncated CMV promoter. In other embodiments, the promoter may be an EFla promoter. In some embodiments, the promoter may be an inducible promoter. Non-limiting exemplary inducible promoters include those inducible by heat shock, light, chemicals, peptides, metals, steroids, antibiotics, or alcohol. In some embodiments, the inducible promoter may be one that has a low basal (non-induced) expression level, such as, e.g., the Tet-On® promoter (Clontech). In some embodiments, the promoter may be a tissue-specific promoter. In some embodiments, the tissue-specific promoter is exclusively or predominantly expressed in liver tissue. Non-limiting exemplary tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-b promoter, Mb promoter, Nphsl promoter, OG-2 promoter, SP-B promoter, SYN1 promoter, and WASP promoter.

### Lipid Nanoparticles (LNPs)

The retron-based editing systems or components thereof (e.g., linear and circular mRNAs; nucleobase editing systems and/or components thereof) described herein may be encapsulated and delivered by lipid nanoparticles (LNPs) and compositions and/or formulations comprising RNA-encapsulated LNPs.

Below describes LNPs that may be used as the payload delivery vehicles contemplated herein, as well as the various ionizable lipids, structural lipids, PEGylated lipids, and phospholipids that may be used to make the herein LNPs for delivery payloads to cells. In addition, below describes additional LNP components that are contemplated, such as targeting moieties and other lipid components.

In one aspect, the present disclosure further provides delivery systems for delivery of a therapeutic payload (e.g., the RNA payloads described herein which may encode a polypeptide of interest, e.g., a nucleobase editing system or a therapeutic protein) disclosed herein. In some embodiments, a delivery system suitable for delivery of the therapeutic payload disclosed herein comprises a lipid nanoparticle (LNP) formulation.

In some embodiments, an LNP of the present disclosure comprises an ionizable lipid, a structural lipid, a PEGylated lipid (aka PEG lipid), and a phospholipid. In alternative embodiments, an LNP comprises an ionizable lipid, a structural lipid, a PEGylated lipid (aka PEG lipid), and a zwitterionic amino acid lipid. In some embodiments, an LNP further comprises a 5th lipid, besides any of the aforementioned lipid components. In some embodiments, the LNP encapsulates one or more elements of the active agent of the present disclosure. In some embodiments, an LNP further comprises a targeting moiety covalently or non-covalently bound to the outer surface of the LNP. In some embodiments, the targeting moiety is a targeting moiety that binds to, or otherwise facilitates uptake by, cells of a particular organ system.

In some embodiments, an LNP has a diameter of at least about 20 nm, 30 nm, 40 nm, 50 nm, 60 nm, 70 nm, 80 nm, or 90 nm. In some embodiments, an LNP has a diameter of less than about 100 nm, 110 nm, 120 nm, 130 nm, 140 nm, 150 nm, or 160 nm. In some embodiments, an LNP has a diameter of less than about 100 nm. In some embodiments, an LNP has a diameter of less than about 90 nm. In some embodiments, an LNP has a diameter of less than about 80 nm. In some embodiments, an LNP has a diameter of about 60-100 nm. In some embodiments, an LNP has a diameter of about 75-80 nm.

In some embodiments, the lipid nanoparticle compositions of the present disclosure are described according to the respective molar ratios of the component lipids in the formulation. As a non-limiting example, the mol-% of the ionizable lipid may be from about 10 mol-% to about 80 mol-%. As a non-limiting example, the mol-% of the ionizable lipid may be from about 20 mol-% to about 70 mol-%. As a non-limiting example, the mol-% of the ionizable lipid may be from about 30 mol-% to about 60 mol-%. As a non-limiting example, the mol-% of the ionizable lipid may be from about 35 mol-% to about 55 mol-%. As a non-limiting example, the mol-% of the ionizable lipid may be from about 40 mol-% to about 50 mol-%.

In some embodiments, the mol-% of the phospholipid may be from about 1 mol-% to about 50 mol-%. In some embodiments, the mol-% of the phospholipid may be from about 2 mol-% to about 45 mol-%. In some embodiments, the mol-% of the phospholipid may be from about 3 mol-% to about 40 mol-%. In some embodiments, the mol-% of the phospholipid may be from about 4 mol-% to about 35 mol-%. In some embodiments, the mol-% of the phospholipid may be from about 5 mol-% to about 30 mol-%. In some embodiments, the mol-% of the phospholipid may be from about 10 mol-% to about 20 mol-%. In some embodiments, the mol-% of the phospholipid may be from about 5 mol-% to about 20 mol-%.

In some embodiments, the mol-% of the structural lipid may be from about 10 mol-% to about 80 mol-%. In some embodiments, the mol-% of the structural lipid may be from about 20 mol-% to about 70 mol-%. In some embodiments, the mol-% of the structural lipid may be from about 30 mol-% to about 60 mol-%. In some embodiments, the mol-% of the structural lipid may be from about 35 mol-% to about 55 mol-%. In some embodiments, the mol-% of the structural lipid may be from about 40 mol-% to about 50 mol-%.

In some embodiments, the mol-% of the PEG lipid may be from about 0.1 mol-% to about 10 mol-%. In some embodiments, the mol-% of the PEG lipid may be from about 0.2 mol-% to about 5 mol-%. In some embodiments, the mol-% of the PEG lipid may be from about 0.5 mol-% to about 3 mol-%. In some embodiments, the mol-% of the PEG lipid may be from about 1 mol-% to about 2 mol-%. In some embodiments, the mol-% of the PEG lipid may be about 1.5 mol-%. In some embodiments, the mol-% of the PEG lipid may be about 2.5 mol-%.

i. Ionizable Lipids

In some embodiments, an LNP disclosed herein comprises an ionizable lipid. In some embodiments, an LNP comprises two or more ionizable lipids.

Described below are a number of exemplary ionizable lipids of the present disclosure.

In some embodiments, an LNP of the present disclosure comprises an ionizable lipid disclosed in one of US 2023/0053437; US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, an LNP described herein comprises a lipid, e.g., an ionizable lipid, disclosed in US Application publication US2017/0119904, which is incorporated by reference herein, in its entirety.

In some embodiments, an LNP described herein comprises a lipid, e.g., an ionizable lipid, disclosed in PCT Application publication WO2021/204179, which is incorporated by reference herein, in its entirety.

In some embodiments, an LNP described herein comprises a lipid, e.g., an ionizable lipid, disclosed in PCT Application WO2022/251665A1, which is incorporated by reference herein, in its entirety. In some embodiments, an LNP described herein comprises an ionizable lipid of Table Z:

TABLE Z
Exemplary Ionizable Lipids

| Compound # | Structure |
|---|---|
| L-1 | 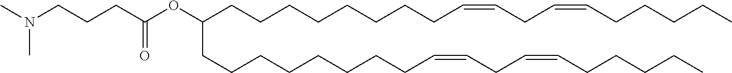 [SMILES: CCCCC/C=C\C/C=C\CCCCCCCCC(CCCCCCCC/C=C\C/C=C\CCCCC)OC(=O)CCCN(C)C] |
| L-2 | 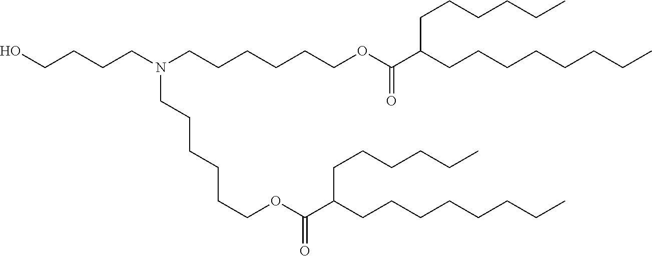 [SMILES: CCCCCCCCC(CCCCCC)C(=O)OCCCCCCN(CCCCO)CCCCCCOC(=O)C(CCCCCC)CCCCCCCC] |
| L-3 | 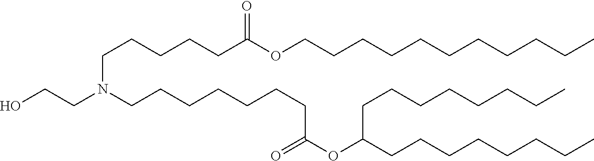 [SMILES: CCCCCCCCCCCOC(=O)CCCCCN(CCO)CCCCCCCC(=O)OC(CCCCCCCC)CCCCCCCC] |
| L-4 | 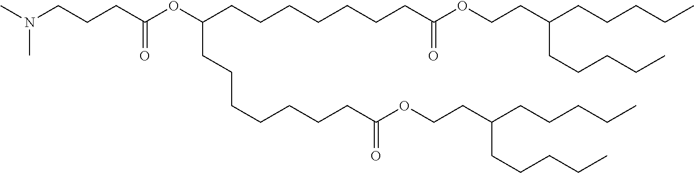 [SMILES: CCCCCC(CCCCC)CCOC(=O)CCCCCCCC(CCCCCCCC(=O)OCCC(CCCCC)CCCCC)OC(=O)CCCN(C)C] |
| L-5 | 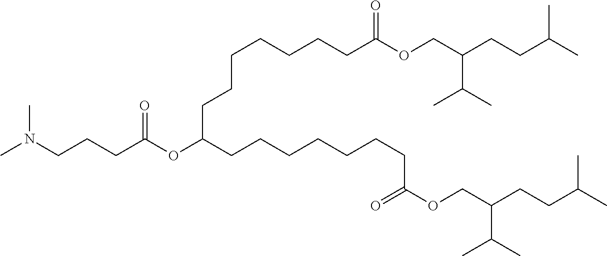 [SMILES: CC(C)CCC(COC(=O)CCCCCCCC(CCCCCCCC(=O)OCC(CCC(C)C)C(C)C)OC(=O)CCCN(C)C)C(C)C] |
| L-6 | 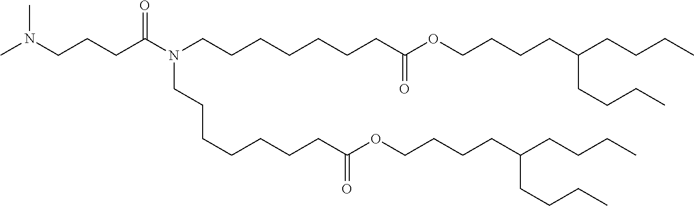 [SMILES: CCCCC(CCCC)CCCCOC(=O)CCCCCCCN(CCCCCCCC(=O)OCCCCC(CCCC)CCCC)C(=O)CCCN(C)C] |
| L-7 | 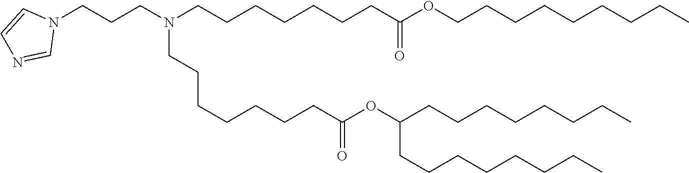 [SMILES: CCCCCCCCCOC(=O)CCCCCCCN(CCCCCCCC(=O)OC(CCCCCCCC)CCCCCCCC)CCCn1ccnc1] |
| L-8 | 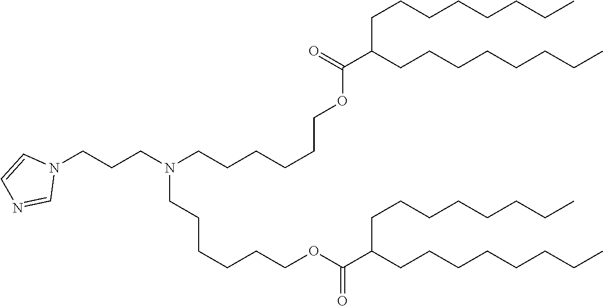 [SMILES: CCCCCCCCC(CCCCCCCC)C(=O)OCCCCCCN(CCCCCCOC(=O)C(CCCCCCCC)CCCCCCCC)CCCn1ccnc1] |
| L-9 | 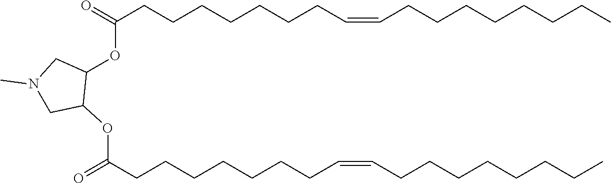 [SMILES: CCCCCCCC/C=C\CCCCCCCC(=O)OC1CN(C)CC1OC(=O)CCCCCCC/C=C\CCCCCCCC] |
| L-10 | 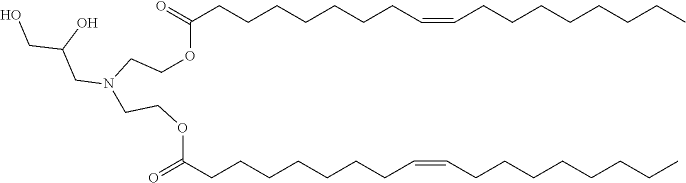 [SMILES: CCCCCCCC/C=C\CCCCCCCC(=O)OCCN(CCOC(=O)CCCCCCC/C=C\CCCCCCCC)CC(O)CO] |

In some embodiments, the ionizable lipid is MC3.

In some embodiments, an LNIP of the present disclosure comprises an ionizable lipid disclosed in PCT Application Publication WO2023044343A1, which is incorporated by reference herein, in its entirety.

### Formula VII-A)

In some embodiments, Lipids of the Disclosure have a structure of Formula (VII-A):

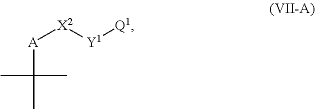

or a pharmaceutically acceptable salt thereof, wherein:
A is —N(—X¹R¹)—, —C(R′)(-L¹-N(R″)R⁶)—, —C(R′)(—OR^{7a})—, —C(R′)(—N(R″)R^{8a})—, —C(R′)(—C(═O)OR^{9a})—, —C(R′)(—C(═O)N(R″)R^{10a})—, or —C(═N—R^{11a})—;
T is —X^{2a}—Y^{1a}-Q^{1a} or —X³—C(═O)OR⁴;
X¹ is optionally substituted C₂-C₆ alkylenyl;
R¹ is —OH, —R^{1a},

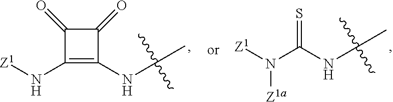

Z¹ is optionally substituted C₁-C₆ alkyl;
Z^{1a} is hydrogen or optionally substituted C₁-C₆ alkyl;
X² and X^{2a} are independently optionally substituted C₂-C₁₄ alkylenyl or optionally substituted C₂-C₁₄ alkenylenyl;
X³ is optionally substituted C₂-C₁₄ alkylenyl or optionally substituted C₂-C₁₄ alkenylenyl;
(i) Y¹ is

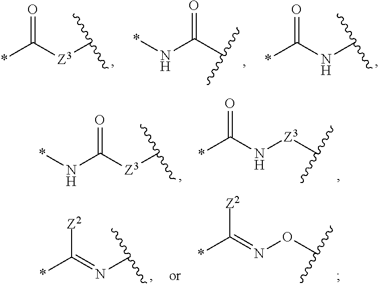

wherein the bond marked with an “*” is attached to X2
   Y^{1a} is

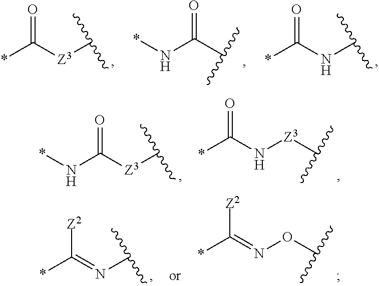

wherein the bond marked with an “*” is attached to X^{2a};
each Z² is independently H or optionally substituted C₁-C₈ alkyl;
each Z³ is independently optionally substituted C₁-C₆ alkylenyl;
Q¹ is —NR²R³, —CH(OR²)(OR³), —CR²═C(R³)(R¹²), or —C(R²)(R³)(R²);
Q^{1a} is —NR²R^{3′}, —CH(OR²)(OR^{3′}), —CR²═C(R³)(R²), or —C(R^{2′})(R^{3′})(R^{12′}); or
(ii) Y¹ is

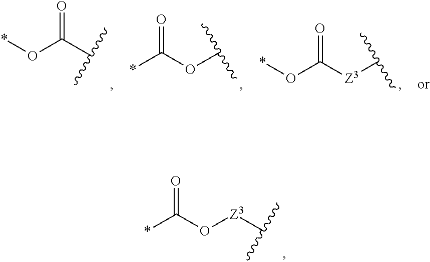

wherein the bond marked with an “*” is attached to X2
Y^{1a} is

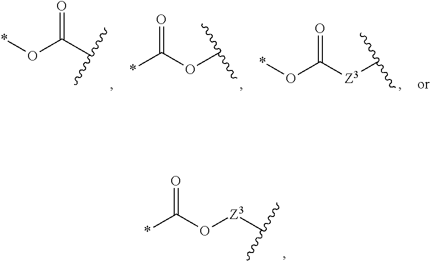

wherein the bond marked with an “*” is attached to X^{2a};
each Z² is independently H or optionally substituted C₁-C₈ alkyl;
each Z³ is independently optionally substituted C₁-C₆ alkylenyl;
Q¹ is —NR²R³;
Q^{1a} is —NR²R^{3′};
R², R³, and R¹² are independently hydrogen, optionally substituted C₁-C₁₄ alkyl, optionally substituted C₂-C₁₄ alkenylenyl, or —(CH₂)ₘ-G-(CH₂)ₙH;
R^{2′}, R^{3′}, and R^{12′} are independently hydrogen, optionally substituted C₁-C₁₄ alkyl, optionally substituted C₂-C₁₄ alkenylenyl, or —(CH₂)ₘ-G-(CH₂)ₙH;
G is a C₃-C₈ cycloalkylenyl;
each m is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
X³ is optionally substituted C₂-C₁₄ alkylenyl;
R⁴ is optionally substituted C₄-C₁₄ alkyl;
L¹ is C₁-C₈ alkylenyl;
R⁶ is C₁-C₆ alkyl, (hydroxy)C₁-C₆ alkyl, or (amino)C₁-C₆ alkyl
R^{7a} is —C(═O)N(R′″)R^{7b}, —C(═S)N(R′″)R^{7b}, —N═C(R^{7b})(R^{7c}), or

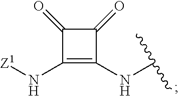

R^{7b} is C₁-C₆ alkyl, (hydroxy)C₁-C₆ alkyl, or (amino)C₁-C₆ alkyl;
R^{7c} is hydrogen or C₁-C₆ alkyl;
R^{8a} is —C(═O)N(R′″)R^{8b}, —C(═S)N(R′″)R^{8b}, —N═C(R^{8b})(R^{8c}), or

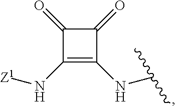

R^{8b} is C₁-C₆ alkyl, (hydroxy)C₁-C₆ alkyl, or (amino)C₁-C₆ alkyl;
R^{8c} is hydrogen or C₁-C₆ alkyl;
R^{9a} is —N═C(R^{9b})(R^{9c});
R^{9b} is C₁-C₆ alkyl, (hydroxy)C₁-C₆ alkyl, or (amino)C₁-C₆ alkyl;
R^{9c} is hydrogen or C₁-C₆ alkyl;
R^{10a} is —N═C(R^{10b})(R^{10c})
R^{10b} is C₁-C₆ alkyl, (hydroxy)C₁-C₆ alkyl, or (amino)C₁-C₆ alkyl;
R^{10c} is hydrogen or C₁-C₆ alkyl;
R^{11a} is —OR^{11b}, —N(R″)R^{11b}, —OC(═O)R^{11b} or —N(R″)C(═O)R^{11b};
R^{11b} is C₁-C₆ alkyl, (hydroxy)C₁-C₆ alkyl, or (amino)C₁-C₆ alkyl;
R′ is hydrogen or C₁-C₆ alkyl;
R″ is hydrogen or C₁-C₆ alkyl; and
R′″ is hydrogen or C₁-C₆ alkyl.

### Formula VIII-A)

In some embodiments, Lipids of the Disclosure have a structure of Formula (VII-A), wherein the Lipids of the Disclosure have a structure of Formula (VIII-A):

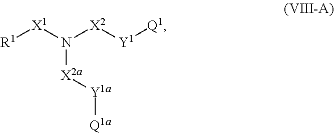

or a pharmaceutically acceptable salt thereof.

### Formula VII-B)

In some embodiments, Lipids of the Disclosure have a structure of Formula (VII-B):

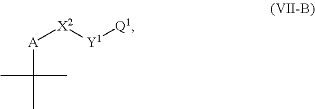

or a pharmaceutically acceptable salt thereof, wherein:
A is —C(R′)(-L¹-N(R″)R⁶)—, —C(R′)(—OR^{7a})—, —C(R′)(—N(R″)R^{8a})—, —C(R′)(—C(═O)OR^{9a})—, —C(R′)(—C(═O)N(R″)R^{10a})—, or —C(═N—R^{11a})—;
T is —X^{2a}—Y^{1a}-Q^{1a} or —X³—C(═O)OR⁴;
X² and X^{2a} are independently optionally substituted C₂-C₁₄ alkylenyl or optionally substituted C₂-C₁₄ alkenylenyl;
X³ is optionally substituted C₁-C₁₄ alkylenyl or optionally substituted C₂-C₁₄ alkenylenyl;
Y¹ is

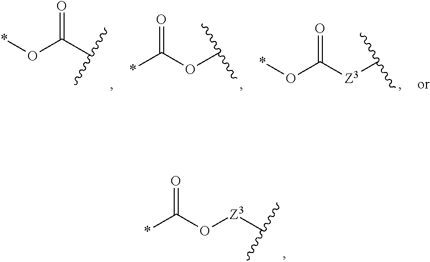

wherein the bond marked with an “*” is attached to X²;
Y^{1a} is

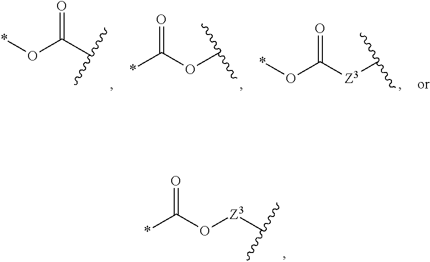

wherein the bond marked with an “*” is attached to X^{2a};
each Z³ is independently optionally substituted C₁-C₆ alkylenyl or optionally substituted C₂-C₁₄ alkenylenyl;
Q¹ is —NR²R³, —CH(OR²)(OR³), —CR²═C(R³)(R¹²), or —C(R²)(R³)(R²);
Q^{1a} is —NR^{2′}R^{3′}, —CH(OR^{2′})(OR³), —CR²═C(R³)(R²), or —C(Rz)(R^{3′})(R^{12′});
R², R³, and R¹² are independently hydrogen, optionally substituted C₁-C₁₄ alkyl, optionally substituted C₂-C₁₄ alkenylenyl, or —(CH₂)ₘ-G-(CH₂)ₙH;
R^{2′}, R^{3′}, and R^{12′} are independently hydrogen, optionally substituted C₁-C₁₄ alkyl, optionally substituted C₂-C₁₄ alkenylenyl, or —(CH₂)ₘ-G-(CH₂)ₙH;
G is a C₃-C₈ cycloalkylenyl;
each m is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
X³ is optionally substituted C₂-C₁₄ alkylenyl;
R⁴ is optionally substituted C₄-C₁₄ alkyl;
L¹ is C₁-C₈ alkylenyl;
R⁶ is (hydroxy)C₁-C₆ alkyl, or (amino)C₁-C₆ alkyl.
R^{7a} is —C(═O)N(R′″)R^{7b}, —C(═S)N(R′″)R^{7b}, —N═C(R^{7b})(R^{7c}),

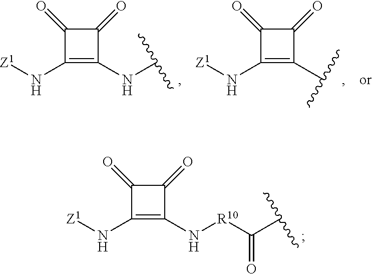

Z¹ is optionally substituted C₁-C₆ alkyl;
R¹⁰ is C₁-C₆ alkylenyl;
R^{7b} is C₁-C₆ alkyl, (hydroxy)C₁-C₆ alkyl, or (amino)C₁-C₆ alkyl;
R^{7c} is hydrogen or C₁-C₆ alkyl;
R^{8a} is —C(═O)N(R′″)R^{8b}, —C(═S)N(R′″)R^{8b}, —N═C(R^{8b})(R^{8c}),

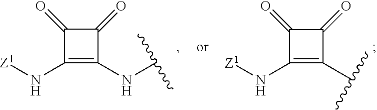

R^{8b} is C₁-C₆ alkyl, (hydroxy)C₁-C₆ alkyl, or (amino)C₁-C₆ alkyl;
R^{8c} is hydrogen or C₁-C₆ alkyl;
R^{9a} is —N═C(R^{9b})(R^{9c});
R^{9b} is C₁-C₆ alkyl, (hydroxy)C₁-C₆ alkyl, or (amino)C₁-C₆ alkyl;
R^{9c} is hydrogen or C₁-C₆ alkyl;
R^{10a} is —N═C(R^{10b})(R^{10c}),
R^{10b} is C₁-C₆ alkyl, (hydroxy)C₁-C₆ alkyl, or (amino)C₁-C₆ alkyl;
R^{10c} is hydrogen or C₁-C₆ alkyl;
R^{11a} is —OR^{11b}, —N(R″)R^{11b}, —OC(═O)R^{11b} or —N(R″)C(═O)R^{11b};
R^{11b} is C₁-C₆ alkyl, (hydroxy)C₁-C₆ alkyl, or (amino)C₁-C₆ alkyl;
R′ is hydrogen or C₁-C₆ alkyl;
R″ is hydrogen or C₁-C₆ alkyl; and
R′″ is hydrogen or C₁-C₆ alkyl.

In some embodiments, Lipids of the Disclosure have a structure of Formula (VII-B), wherein A is —C(R′)(-L¹-N(R″)R⁶)—.

In some embodiments, Lipids of the Disclosure have a structure of Formula (VII-B), wherein A is —C(R′)(—OR^{7a})—.

In some embodiments, Lipids of the Disclosure have a structure of Formula (VII-B), wherein A is —C(R′)(—N(R″)R^{8a}).

In some embodiments, Lipids of the Disclosure have a structure of Formula (VII-B), wherein A is —C(R′)(—C(═O)OR^{9a}).

In some embodiments, Lipids of the Disclosure have a structure of Formula (VII-B), wherein A is —C(R′)(—C(═O)N(R″)R^{10a})—.

In some embodiments, Lipids of the Disclosure have a structure of Formula (VII-B), wherein A is —C(═N—R^{11a})—.

In some embodiments, Lipids of the Disclosure have a structure of Formula (VII-B), wherein T is —X^{2a}—Y^{1a}-Q^{1a}.

In some embodiments, Lipids of the Disclosure have a structure of Formula (VII-B), wherein T is —X³—C(═O)OR⁴.

In some embodiments, Lipids of the Disclosure have a structure of Formula (VII-B), wherein X² and/or X^{2a} are/is optionally substituted C₂-C₁₄ alkylenyl (e.g., C₂-C₁₀ alkylenyl, C₂-C₈ alkylenyl, C₂, C₃, C₄, C₅, C₆, C₇, or C₈ alkylenyl). In some embodiments, Lipids of the Disclosure have a structure of Formula (VII-B), wherein X² is C₂-C₁₄ alkylenyl. In some embodiments, Lipids of the Disclosure have a structure of Formula (VII-B), wherein X^{2a} is C₂-C₁₄alkylenyl

In some embodiments, Lipids of the Disclosure have a structure of Formula (VII-B), wherein Y¹ and/or Y^{1a} are/is

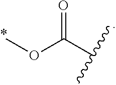 [SMILES: *C(=O)OC]

In some embodiments, Lipids of the Disclosure have a structure of Formula (VII-B), wherein Y¹ is

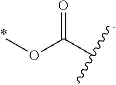 [SMILES: *C(=O)OC]

In some embodiments, Lipids of the Disclosure have a structure of Formula (VII-B), wherein Y^{1a} is

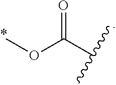 [SMILES: *C(=O)OC]

In some embodiments, Lipids of the Disclosure have a structure of Formula (VII-B), wherein Y¹ and/or Y^{1a} are/is

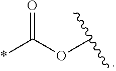 [SMILES: *OC(*)=O]

In some embodiments, Lipids of the Disclosure have a structure of Formula (VII-B), wherein Y¹ is

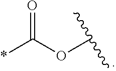 [SMILES: *OC(*)=O]

In some embodiments, Lipids of the Disclosure have a structure of Formula (VII-B), wherein Y^{1a} is

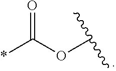 [SMILES: *OC(*)=O]

In some embodiments, Lipids of the Disclosure have a structure of Formula (VII-B), wherein Y¹ and/or Y^{1a} are/is

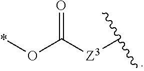

In some embodiments, Lipids of the Disclosure have a structure of Formula (VII-B), wherein Y¹ is

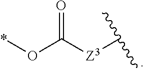

In some embodiments, Lipids of the Disclosure have a structure of Formula (VII-B), wherein Y^{1a} is

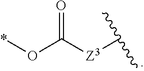

In some embodiments, Lipids of the Disclosure have a structure of Formula (VII-B), wherein Y¹ and/or Y^{1a} are/is

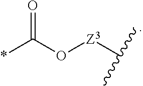

In some embodiments, Lipids of the Disclosure have a structure of Formula (VII-B), wherein Y¹ is

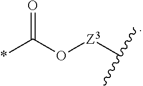

In some embodiments, Lipids of the Disclosure have a structure of Formula (VII-B), wherein Y^{1a} is

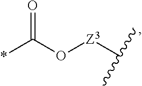

In some embodiments, Lipids of the Disclosure have a structure of Formula (VII-B), wherein Q¹ and/or Q^{1a} are/is —C(R″)(R³)(R^{12′}). In some embodiments, Lipids of the Disclosure have a structure of Formula (VII-B), wherein Q¹ is —C(R²)(R³)(R^{12′}). In some embodiments, Lipids of the Disclosure have a structure of Formula (VII-B), wherein Q^{1a} is —C(R^{2′})(R^{3′})(R^{12′}).

In some embodiments, Lipids of the Disclosure have a structure of Formula (VII-B), wherein X³ is optionally substituted C₁-C₁₄ alkylenyl (e.g., C₁-C₆, C₁-C₄ alkylenyl). In some embodiments, Lipids of the Disclosure have a structure of Formula (VII-B), wherein X³ is C₁-C₁₄ alkylenyl.

In some embodiments, Lipids of the Disclosure have a structure of Formula (VII-B), wherein R², R³, R¹², R^{2′}, R^{3′}, and/or R^{12′} are hydrogen. In some embodiments, Lipids of the Disclosure have a structure of Formula (VII-B), wherein R² is hydrogen. In some embodiments, Lipids of the Disclosure have a structure of Formula (VII-B), wherein R³ is hydrogen. In some embodiments, Lipids of the Disclosure have a structure of Formula (VII-B), wherein R¹² is hydrogen. In some embodiments, Lipids of the Disclosure have a structure of Formula (VII-B), wherein R^{2″} is hydrogen. In some embodiments, Lipids of the Disclosure have a structure of Formula (VII-B), wherein R^{3′} is hydrogen. In some embodiments, Lipids of the Disclosure have a structure of Formula (VII-B), wherein R^{12′} is hydrogen.

In some embodiments, Lipids of the Disclosure have a structure of Formula (VII-B), wherein R², R³, R¹², R^{2′}, R^{3′}, and/or R^{12′} are optionally substituted C₁-C₁₄ alkyl (e.g., C₄-C₁₀ alkyl, C₅, C₆. C₇. C₈, C₉ alkyl). In some embodiments, Lipids of the Disclosure have a structure of Formula (VII-B), wherein R² is C₄-C₁₀ alkyl. In some embodiments, Lipids of the Disclosure have a structure of Formula (VII-B), wherein R³ is C₄-C₁₀ alkyl. In some embodiments, Lipids of the Disclosure have a structure of Formula (VII-B), wherein R¹² is C₄-C₁₀ alkyl. In some embodiments, Lipids of the Disclosure have a structure of Formula (VII-B), wherein R^{2′} is C₄-C₁₀ alkyl. In some embodiments, Lipids of the Disclosure have a structure of Formula (VII-B), wherein R^{3′} is C₄-C₁₀ alkyl. In some embodiments, Lipids of the Disclosure have a structure of Formula (VII-B), wherein R^{12′} is C₄-C₁₀ alkyl.

In some embodiments, Lipids of the Disclosure have a structure of Formula (VII-B), wherein R⁴ is optionally substituted C₄-C₁₄ alkyl (e.g., C₈-C₁₄ alkyl, linear C₈-C₁₄ alkyl, C₈, C₉, C₁₀, C₁₁, C₁₂, C₁₃, or C₁₄ alkyl). In some embodiments, Lipids of the Disclosure have a structure of Formula (VII-B), wherein R⁴ is linear C₈-C₁₄ alkyl. In some embodiments, Lipids of the Disclosure have a structure of Formula (VII-B), wherein R⁴ is linear Cu alkyl.

In some embodiments, Lipids of the Disclosure have a structure of Formula (VII-B), wherein L¹ is C₁-C₃ alkylenyl.

In some embodiments, Lipids of the Disclosure have a structure of Formula (VII-B), wherein R⁶is (hydroxy)C₁-C₆ alkyl.

In some embodiments, Lipids of the Disclosure have a structure of Formula (VII-B), wherein R^{7a} is

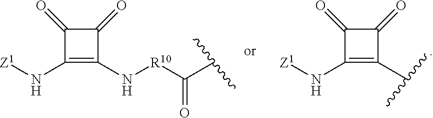

In some embodiments, Lipids of the Disclosure have a structure of Formula (VII-B), wherein R^{7a} is

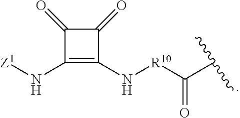

In some embodiments, Lipids of the Disclosure have a structure of Formula (VII-B), wherein R^{7a} is

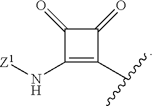

In some embodiments, Lipids of the Disclosure have a structure of Formula (VII-B), wherein R^{7a} is selected from the group consisting of —C(═O)N(R′)R^{7b}, —C(═S)N(R′″)R^{7b}, and —N═C(R^{7b})(R^{7c}). In some embodiments, Lipids of the Disclosure have a structure of Formula (VII-B), wherein R^{7a} is —C(═O)N(R′)R^{7b}. In some embodiments, Lipids of the Disclosure have a structure of Formula (VII-B), wherein R^{7a} is —C(═S)N(R′)R⁷¹. In some embodiments, Lipids of the Disclosure have a structure of Formula (VII-B), wherein R^{7a} is —N═C(R^{7b})(R^{7c}).

In some embodiments, Lipids of the Disclosure have a structure of Formula (VII-B), wherein R^{8a} is selected from the group consisting of —C(═O)N(R′)R^{8b}, —C(═S)N(R′)R^{8b}, and —N═C(R^{8b})(R^{8c}). In some embodiments, Lipids of the Disclosure have a structure of Formula (VII-B), wherein R^{8a} is —C(═O)N(R′)R^{8b}. In some embodiments, Lipids of the Disclosure have a structure of Formula (VII-B), wherein R^{8a} is —C(═S)N(R′)R^{8b}. In some embodiments, Lipids of the Disclosure have a structure of Formula (VII-B), wherein R^{8a} is —N═C(R^{8b})(R^{8c}).

In some embodiments, Lipids of the Disclosure have a structure of Formula (VII-B), wherein R^{8a} is

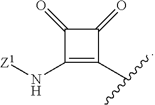

In some embodiments, Lipids of the Disclosure have a structure of Formula (VII-B), wherein R^{9a} is (hydroxy)C₁-C₆ alkyl.

In some embodiments, Lipids of the Disclosure have a structure of Formula (VII-B), wherein R^{10b} is (amino)C₁-C₆ alkyl.

In some embodiments, Lipids of the Disclosure have a structure of Formula (VII-B), wherein R^{11a} is —OR^{11b} or —OC(═O)R^{11b}. In some embodiments, Lipids of the Disclosure have a structure of Formula (VII-B), wherein R^{11a} is —OR^{11b}. In some embodiments, Lipids of the Disclosure have a structure of Formula (VII-B), wherein R^{11a} is —OC(═O)R^{11b}.

In some embodiments, Lipids of the Disclosure have a structure of Formula (VII-B), wherein R^{11a} is —N(R″)R^{11b} or —N(R″)C(═O)R^{11b}. In some embodiments, Lipids of the Disclosure have a structure of Formula (VII-B), wherein R^{11a} is —N(R″)R^{11b}. In some embodiments, Lipids of the Disclosure have a structure of Formula (VII-B), wherein R^{11a} is —N(R″)C(═O)R^{11b}.

In some embodiments, Lipids of the Disclosure have a structure of Formula (VII-B), wherein R^{11b} is (amino)C₁-C₆ alkyl.

### Formula III-C)

In some embodiments, Lipids of the Disclosure have a structure of Formula (III-C):

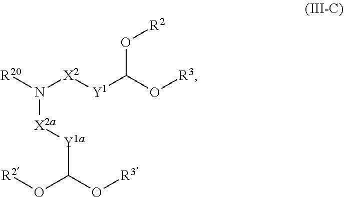

or a pharmaceutically acceptable salt thereof, wherein
R²⁰ is C₁-C₆ alkylenyl-NR^{20′}C(O)OR^{20″};
R^{20′} is hydrogen or optionally substituted C₁-C₆ alkyl;
R^{20″} is optionally substituted C₁-C₆ alkyl, phenyl, or benzyl;
Z¹ is optionally substituted C₁-C₆ alkyl;
X² and X^{2a} are independently optionally substituted C₂-C₁₄ alkylenyl;
Y¹ and Y^{1a} are independently

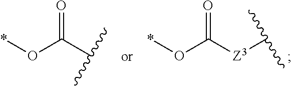

wherein the bond marked with an “*” is attached to X² or X^{2a};
Z³ is independently optionally substituted C₂-C₆ alkylenyl;
R² and R³ are independently optionally substituted C₄-C₁₄ alkyl; and
R² and R^{3′} are independently optionally substituted C₄-C₁₄ alkyl.

In some embodiments, Lipids of the Disclosure have a structure of Formula (III-C), wherein R²⁰ is —CH₂CH₂CH₂NHC(O)O-t-butyl or —CH₂CH₂CH₂NHC(O)O-benzyl. In some embodiments, Lipids of the Disclosure have a structure of Formula (III-C), wherein R²⁰ is —CH₂CH₂CH₂NHC(O)O-t-butyl. In some embodiments, Lipids of the Disclosure have a structure of Formula (III-C), wherein R²⁰ is —CH₂CH₂CH₂NHC(O)O-benzyl.

In some embodiments, Lipids of the Disclosure have a structure of Formula (III-C), wherein X² and X^{2a} are independently C₄-C₈ alkylenyl (e.g., C₅, C₆, C₇ alkylenyl). In some embodiments, Lipids of the Disclosure have a structure of Formula (III-C), wherein X² is C₆ alkyl. In some embodiments, Lipids of the Disclosure have a structure of Formula (III-C), wherein X^{2a} is C₆ alkyl

In some embodiments, Lipids of the Disclosure have a structure of Formula (III-C), wherein Y¹ and Y^{1a} are

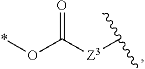

wherein Z³ is C₂-C₄alkylenyl (e.g., C₂ alkylenyl). In some embodiments, Lipids of the Disclosure have a structure of Formula (III-C), wherein Y¹ is

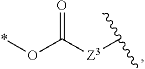

wherein Z³ is C₂-C₄alkylenyl (e.g., C₂ alkylenyl). In some embodiments, Lipids of the Disclosure have a structure of Formula (III-C), wherein Y^{1a} is

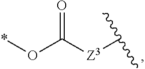

wherein Z³ is C₂-C₄alkylenyl (e.g., C2 alkylenyl).

In some embodiments, Lipids of the Disclosure have a structure of Formula (III-C), wherein R2, R3, R2′ and R3′ are independently optionally substituted C4-C10 alkyl (e.g., C6-C9alkyl, C6, C7, C8, C9 alkyl). In some embodiments, Lipids of the Disclosure have a structure of Formula (III-C), wherein R2 is C6-C9alkyl. In some embodiments, Lipids of the Disclosure have a structure of Formula (III-C), wherein R3 is C6-C9alkyl. In some embodiments, Lipids of the Disclosure have a structure of Formula (III-C), wherein R2′ is C6-C9alkyl. In some embodiments, Lipids of the Disclosure have a structure of Formula (III-C), wherein R^{3′} is C₆-C₉alkyl.

### Formula (III-D)

In some embodiments, Lipids of the Disclosure have a structure of Formula (III-D):

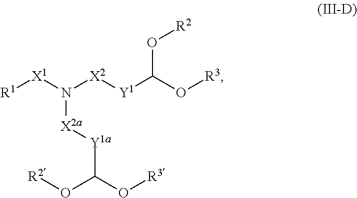

or a pharmaceutically acceptable salt thereof, wherein
R¹ is —OH;
X¹ is optionally substituted C₄ alkylenyl;
X² and X^{2a} are independently optionally substituted C₂-C₁₄ alkylenyl;
Y¹ and Y^{1a} are independently

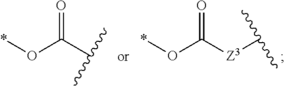

Z³ is independently optionally substituted C₂-C₆ alkylenyl;
R² and R³ are independently optionally substituted C₄-C₁₄ alkyl or C₁-C₂ alkyl substituted with optionally substituted cyclopropyl; or
R², and R³, are independently optionally substituted C₄-C₁₄ alkyl or C₁-C₂ alkyl substituted with optionally substituted cyclopropyl.

In some embodiments, Lipids of the Disclosure have a structure of Formula (III-D), wherein X¹ is C₄ alkylenyl.

In some embodiments, Lipids of the Disclosure have a structure of Formula (III-D), wherein X² and X^{2a} are independently optionally substituted C₄-C₁₀ alkylenyl (e.g., C₅, C₆, C₇, C₈, C₉, or C₁₀ alkylenyl). In some embodiments, Lipids of the Disclosure have a structure of Formula (III-D), wherein X² is C₄-C₁₀ alkylenyl. In some embodiments, Lipids of the Disclosure have a structure of Formula (III-D), wherein X^{2a} is C₄-C₁₀ alkylenyl.

In some embodiments, Lipids of the Disclosure have a structure of Formula (III-D), wherein Y¹ and Y^{1a} are independently

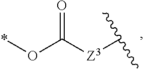

wherein Z³ is independently C₂-C₄ alkylenyl (e.g., C₂, C₄ alkylenyl).

In some embodiments, Lipids of the Disclosure have a structure of Formula (III-D), wherein R², R³, R^{2′} and R³, are independently C₆-C₁₄ alkyl (e.g., C₆, C₇, C₈, C₉, C₁₀, C₁₁, C₁₂, C₁₃, or C₁₄ alkyl) or C₁-C₂ alkyl substituted with optionally substituted cyclopropyl. In some embodiments, Lipids of the Disclosure have a structure of Formula (III-D), wherein R², R³, R^{2′} and R³, are independently C₆-C₁₄ alkyl (e.g., C₆, C₇, C₈, C₉, C₁₀, C₁₁, C₁₂, C₁₃, or C₁₄ alkyl). In some embodiments, Lipids of the Disclosure have a structure of Formula (III-D), wherein R² is C₆-C₁₄ alkyl. In some embodiments, Lipids of the Disclosure have a structure of Formula (III-D), wherein R³ is C₆-C₁₄ alkyl. In some embodiments, Lipids of the Disclosure have a structure of Formula (III-D), wherein R^{2′} is C₆-C₁₄ alkyl. In some embodiments, Lipids of the Disclosure have a structure of Formula (III-D), wherein R³ is C₆-C₁₄ alkyl. In some embodiments, Lipids of the Disclosure have a structure of Formula (III-D), wherein R² is C₁-C₂ alkyl substituted with substituted cyclopropyl. In some embodiments, Lipids of the Disclosure have a structure of Formula (III-D), wherein R³ is C_{L}-C₂ alkyl substituted with substituted cyclopropyl. In some embodiments, Lipids of the Disclosure have a structure of Formula (III-D), wherein R^{2′} is C₁-C₂ alkyl substituted with substituted cyclopropyl. In some embodiments, Lipids of the Disclosure have a structure of Formula (III-D), wherein R^{3′} is C₁-C₂ alkyl substituted with substituted cyclopropyl

In some embodiments, Lipids of the Disclosure have a structure of Formula (III-D), wherein R², R³, R^{2′} and R^{3′} are independently C₁-C₂ alkyl substituted with cyclopropylene-(C₁-C⁶alkylenyl optionally substituted with cyclopropylene substituted with C₁-C₆alkyl). In some embodiments, Lipids of the Disclosure have a structure of Formula (III-D), wherein R² is C₁-C₂ alkyl substituted with cyclopropylene-(C₁-C₆alkylenyl optionally substituted with cyclopropylene substituted with C₁-C₆alkyl). In some embodiments, Lipids of the Disclosure have a structure of Formula (III-D), wherein R³ is C₁-C₂ alkyl substituted with cyclopropylene-(C₁-C⁶alkylenyl optionally substituted with cyclopropylene substituted with C₁-C₆alkyl). In some embodiments, Lipids of the Disclosure have a structure of Formula (III-D), wherein R^{2′} is C₁-C₂ alkyl substituted with cyclopropylene-(C₁-C⁶alkylenyl optionally substituted with cyclopropylene substituted with C₁-C₆alkyl). In some embodiments, Lipids of the Disclosure have a structure of Formula (III-D), wherein R^{3′} is C₁-C₂ alkyl substituted with cyclopropylene-(C₁-C⁶alkylenyl optionally substituted with cyclopropylene substituted with C₁-C₆alkyl).

### Formula (III-E)

In some embodiments, Lipids of the Disclosure have a structure of Formula (III-E):

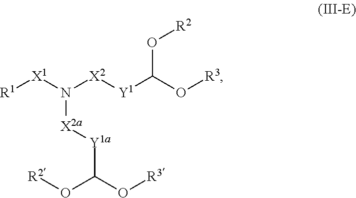

or a pharmaceutically acceptable salt thereof, wherein
R¹ is —OH;
X¹ is branched C₂-C₈ alkylenyl
X² and X^{2a} are independently optionally substituted C₂-C₁₄ alkylenyl;
Y¹ and Y^{1a} are independently

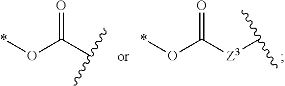

Z³ is independently optionally substituted C₂-C₆ alkylenyl;
R² and R³ are independently optionally substituted C₄-C₁₄ alkyl;
R^{2′} and R³ are independently optionally substituted C₄-C₁₄ alkyl.

In some embodiments, Lipids of the Disclosure have a structure of Formula (III-E), wherein X¹ is branched C₆ alkylenyl.

In some embodiments, Lipids of the Disclosure have a structure of Formula (III-E), wherein X² and X^{2a} are independently C₄-C₁₀ alkylenyl (e.g., C₆, C₇, C₈ alkylenyl). In some embodiments, Lipids of the Disclosure have a structure of Formula (III-E), wherein X² is C₄-C₁₀ alkylenyl. In some embodiments, Lipids of the Disclosure have a structure of Formula (III-E), wherein X^{2a} is C₄-C₁₀ alkylenyl

In some embodiments, Lipids of the Disclosure have a structure of Formula (III-E), wherein Y¹ and Y^{1a} are

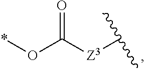

wherein Z³ is independently optionally substituted C₂ alkylenyl. In some embodiments, Lipids of the Disclosure have a structure of Formula (III-E), wherein Y¹ is

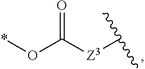

wherein Z³ is independently optionally substituted C₂ alkylenyl. In some embodiments, Lipids of the Disclosure have a structure of Formula (III-E), wherein Y^{1a} is

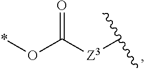

wherein Z³ is independently optionally substituted C₂ alkylenyl.

In some embodiments, Lipids of the Disclosure have a structure of Formula (III-E), wherein R², R³, R^{2′} and R^{3′} are independently C₆-C₁₂ alkyl (e.g., C₉ alkyl) or C₄-C₁₀ alkyl (e.g., C₄, C₆ alkyl) optionally substituted with C₂-C₈alkenylene (e.g., C₄, C₆ alkenylene). In some embodiments, Lipids of the Disclosure have a structure of Formula (III-E), wherein R² is C₆-C₁₂ alkyl. In some embodiments, Lipids of the Disclosure have a structure of Formula (III-E), wherein R³ is C₆-C₁₂ alkyl. In some embodiments, Lipids of the Disclosure have a structure of Formula (III-E), wherein R² is C₆-C₁₂ alkyl. In some embodiments, Lipids of the Disclosure have a structure of Formula (III-E), wherein R^{3′} is C₆-C₁₂ alkyl. In some embodiments, Lipids of the Disclosure have a structure of Formula (III-E), wherein R² is C₄-C₁₀ alkyl optionally substituted with C₂-C₈alkenylene. In some embodiments, Lipids of the Disclosure have a structure of Formula (III-E), wherein R³ is C₄-C₁₀ alkyl optionally substituted with C₂-C₈alkenylene. In some embodiments, Lipids of the Disclosure have a structure of Formula (III-E), wherein R^{2′} is C₄-C₁₀ alkyl optionally substituted with C₂-C₈alkenylene. In some embodiments, Lipids of the Disclosure have a structure of Formula (III-E), wherein R^{3′} is C₄-C₁₀ alkyl optionally substituted with C₂-C₈alkenylene.

### Formula III-F)

In some embodiments, Lipids of the Disclosure have a structure of Formula (III-F):

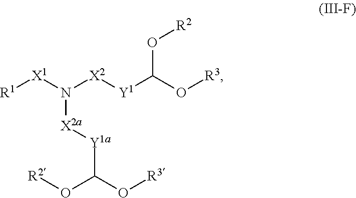

or a pharmaceutically acceptable salt thereof, wherein
R¹ is —OH;
X¹ is optionally substituted C₂-C₆ alkylenyl;
X² and X^{2a} are independently optionally substituted C₂-C₁₄ alkylenyl;
each of Y¹ and Y^{1a} is a bond;
R² and R³ are independently optionally substituted C₄-C₁₄ alkyl; and
R^{2′} and R^{3′} are independently optionally substituted C₄-C₁₄ alkyl.

In some embodiments, Lipids of the Disclosure have a structure of Formula (III-E), wherein X¹ is C₄ alkylenyl.

In some embodiments, Lipids of the Disclosure have a structure of Formula (III-E), wherein X² and X^{2a} are independently C₄-C₁₀ alkylenyl (e.g., C₆-C₈ alkylenyl, C₆, C₇, C₈ alkylenyl). In some embodiments, Lipids of the Disclosure have a structure of Formula (III-E), wherein X² is C₄-C₁₀ alkylenyl. In some embodiments, Lipids of the Disclosure have a structure of Formula (III-E), wherein X^{2a} is C₄-C₁₀ alkylenyl.

In some embodiments, Lipids of the Disclosure have a structure of Formula (III-E), wherein R², R³, R^{2′} and R^{3′} are independently C₆-C₁₀ alkyl (e.g., C₇, C₈ alkyl). In some embodiments, Lipids of the Disclosure have a structure of Formula (III-E), wherein R² is C₆-C₁₀ alkyl. In some embodiments, Lipids of the Disclosure have a structure of Formula (III-E), wherein R³ is C₆-C₁₀ alkyl. In some embodiments, Lipids of the Disclosure have a structure of Formula (III-E), wherein R^{2′} is C₆-C₁₀ alkyl. In some embodiments, Lipids of the Disclosure have a structure of Formula (III-E), wherein R^{3′} is C₆-C₁₀ alkyl.

### Formula VIII-B)

In some embodiments, Lipids of the Disclosure have a structure of Formula (VIII-B):

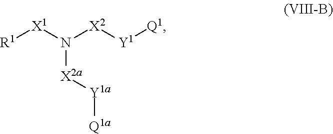

or a pharmaceutically acceptable salt thereof, wherein:
X¹ is a bond,
R¹ is C₁-C₆ alkyl,
X² is C₂-C₆ alkylenyl,
X^{2a} is C₂-C₁₄ alkylenyl,
wherein X² or X^{2a} is substituted with OH or C₁₋₄alkylenyl-OH,
Y¹ is

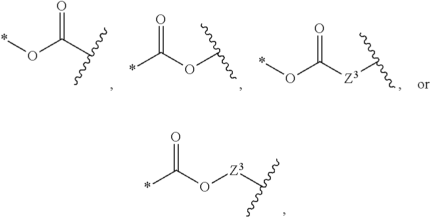

wherein the bond marked with an “*” is attached to X²;
Y^{1a} is

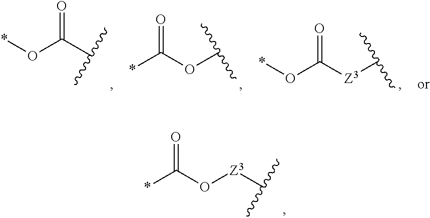

wherein the bond marked with an “*” is attached to X^{2a};
each Z³ is independently optionally substituted C₁-C₆ alkylenyl or optionally substituted C₂-C₁₄ alkenylenyl;
Q¹ is —C(R²)(R³)(R¹²);
Q^{1a} is —C(R^{2′})(R″)(R^{12′});
R², R³, and R¹² are independently hydrogen, optionally substituted C₁-C₁₄ alkyl, or optionally substituted C₂-C₁₄ alkenylenyl, and
R^{2′}, R^{3′}, and R^{12′} are independently hydrogen, optionally substituted C₁-C₁₄ alkyl, or optionally substituted C₂-C₁₄ alkenylenyl.

In some embodiments, Lipids of the Disclosure have a structure of Formula (VIII-B), wherein R¹ is methyl.

In some embodiments, Lipids of the Disclosure have a structure of Formula (VIII-B), wherein X² is C₄, C₅, or C₆ alkylenyl.

In some embodiments, Lipids of the Disclosure have a structure of Formula (VIII-B), wherein X^{2a} is C₄-C₈ alkylenyl (e.g., C₅, C₆, or C₇ alkylenyl).

In some embodiments, Lipids of the Disclosure have a structure of Formula (VIII-B), wherein Y¹ is

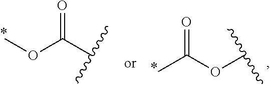

### and Y^{1a} is

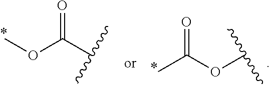

In some embodiments, Lipids of the Disclosure have a structure of Formula (VIII-B), wherein Y¹ is

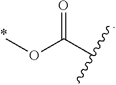 [SMILES: *C(=O)OC]

In some embodiments, Lipids of the Disclosure have a structure of Formula (VIII-B), wherein Y¹ is

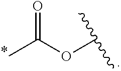 [SMILES: *OC(C)=O]

In some embodiments, Lipids of the Disclosure have a structure of Formula (VIII-B), wherein Y^{1a} is

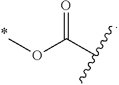 [SMILES: *C(=O)OC]

In some embodiments, Lipids of the Disclosure have a structure of Formula (VIII-B), wherein Y^{1a} is

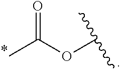 [SMILES: *OC(C)=O]

In some embodiments, Lipids of the Disclosure have a structure of Formula (VIII-B), wherein R², R³, R¹², R^{2′}, R^{3′}, and R^{12′} are independently hydrogen or C₅-C₁₂ alkyl (e.g., C₆, C₇, C₈, C₉, C₁₀, C₁₁ alkyl). In some embodiments, Lipids of the Disclosure have a structure of Formula (VIII-B), wherein R² is hydrogen. In some embodiments, Lipids of the Disclosure have a structure of Formula (VIII-B), wherein R³ is hydrogen. In some embodiments, Lipids of the Disclosure have a structure of Formula (VIII-B), wherein R^{2′} is hydrogen. In some embodiments, Lipids of the Disclosure have a structure of Formula (VIII-B), wherein R^{3′} is hydrogen. In some embodiments, Lipids of the Disclosure have a structure of Formula (VIII-B), wherein R² is C₅-C₁₂ alkyl. In some embodiments, Lipids of the Disclosure have a structure of Formula (VIII-B), wherein R³ is C₅-C₁₂ alkyl. In some embodiments, Lipids of the Disclosure have a structure of Formula (VIII-B), wherein R^{2′} is C₅-C₁₂ alkyl. In some embodiments, Lipids of the Disclosure have a structure of Formula (VIII-B), wherein R^{3′} is C₅-C₁₂ alkyl.

### Formula (X)

In some embodiments, Lipids of the Disclosure have a structure of Formula (X):

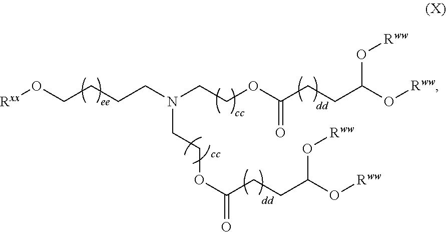

or a pharmaceutically acceptable salt thereof, wherein
each cc is independently selected from 3 to 9;
   R^{xx} is selected from hydrogen and optionally substituted C₁-C₆ alkyl; and
   (i) ee is 1,
      each dd is independently selected from 1 to 4; and
   each R^{ww} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any —(CH₂)₂— of the C₄-C₁₄ alkyl can be optionally replaced with C₂-C₆ cycloalkylenyl;
   (ii) ee is 0,
      each dd is 1; and
      each R^{ww} is linear C₄-C₁₂ alkyl.

In some embodiments, Lipids of the Disclosure have a structure of Formula (X), wherein R^{xx} is H. In some embodiments, Lipids of the Disclosure have a structure of Formula (X), wherein R^{xx} is optionally substituted C₁-C₆ alkyl. In some embodiments, Lipids of the Disclosure have a structure of Formula (X), wherein R^{xx} is C₁ alkyl. In some embodiments, Lipids of the Disclosure have a structure of Formula (X), wherein R^{xx} is C₂ alkyl. In some embodiments, Lipids of the Disclosure have a structure of Formula (X), wherein R^{xx} is C₃ alkyl. In some embodiments, Lipids of the Disclosure have a structure of Formula (X), wherein R^{xx} is C₄ alkyl. In some embodiments, Lipids of the Disclosure have a structure of Formula (X), wherein R^{xx} is C₅ alkyl. In some embodiments, Lipids of the Disclosure have a structure of Formula (X), wherein R^{xx} is C₆ alkyl.

In some embodiments, Lipids of the Disclosure have a structure of Formula (X), wherein each R^{ww} is independently selected from the group consisting of C₄-C₁₄ alkyl, branched C₄-C₁₂ alkenyl, C₄-C₁₂ alkenyl comprising at least two double bonds, and C₉-C₁₂ alkenyl, wherein any —(CH₂)₂— of the C₄-C₁₄ alkyl can be optionally replaced with C₂-C₆ cycloalkylenyl. In some embodiments, Lipids of the Disclosure have a structure of Formula (X), wherein each R^{ww} is C₄-C₁₄ alkyl, wherein any —(CH₂)₂— of the C₄-C₁₄ alkyl can be optionally replaced with C₂-C₆ cycloalkylenyl. In some embodiments, Lipids of the Disclosure have a structure of Formula (X), wherein each R^{ww} is C₄-C₁₄ alkyl, wherein any —(CH₂)₂— of the C₄-C₁₄ alkyl can be optionally replaced with cyclopropylene. In some embodiments, Lipids of the Disclosure have a structure of Formula (X), wherein each R^{ww} is branched C₄-C₁₂ alkenyl. In some embodiments, Lipids of the Disclosure have a structure of Formula (X), wherein each R^{ww} is C₄-C₁₂ alkenyl comprising at least two double bonds. In some embodiments, Lipids of the Disclosure have a structure of Formula (X), wherein each R^{ww} is C₉-C₁₂ alkenyl. In some embodiments, Lipids of the Disclosure have a structure of Formula (X), wherein each R^{ww} is linear C₄-C₁₂ alkyl. In some embodiments, Lipids of the Disclosure have a structure of Formula (X), wherein each R^{ww} is independently selected from the group consisting of C₆-C₁₄ alkyl, branched C₈-C₁₂ alkenyl, C₈-C₁₂ alkenyl comprising at least two double bonds, and C₉-C₁₂ alkenyl, wherein any —(CH₂)₂— of the C₆-C₁₄ alkyl can be optionally replaced with cyclopropylene. In some embodiments, Lipids of the Disclosure have a structure of Formula (X), wherein each R^{ww} is C₆-C₁₄ alkyl, wherein any —(CH₂)₂— of the C₆-C₁₄ alkyl can be optionally replaced with cyclopropylene. In some embodiments, Lipids of the Disclosure have a structure of Formula (X), wherein each R^{ww} is branched C₈-C₁₂ alkenyl, e.g., (linear or branched C₃-C₆ alkylenyl)-(branched C₅-C₇alkenyl), e.g., (branched C₅ alkylenyl)-(branched C₅alkenyl), e.g.,

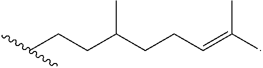

In some embodiments, Lipids of the Disclosure have a structure of Formula (X), wherein each R^{ww} is C₈-C₁₂ alkenyl comprising at least two double bonds. In some embodiments, Lipids of the Disclosure have a structure of Formula (X), wherein each R^{ww} is C₉-C₁₂ alkenyl.

In some embodiments, Lipids of the Disclosure have a structure of Formula (X), wherein each R^{ww} is independently selected from the group consisting of C₆-C₁₄ alkyl (e.g., C₆, C₈, C₉, C₁₀, C₁₁, C₁₃ alkyl), wherein any —(CH₂)₂— of the C₆-C₁₄ alkyl can be optionally replaced with cyclopropylene.

In some embodiments, Lipids of the Disclosure have a structure of Formula (X), wherein each R′ is independently branched C₈-C₁₂ alkenyl (e.g., branched C₁₀ alkenyl).

In some embodiments, Lipids of the Disclosure have a structure of Formula (X), wherein each R^{ww} is independently C₈-C₁₂ alkenyl comprising at least two double bonds (e.g., C₉ or C₁₀ alkenyl comprising two double bonds).

In some embodiments, Lipids of the Disclosure have a structure of Formula (X), wherein each R^{ww} is independently (C₁ alkylenyl)-(cyclopropylene-C₆ alkyl) or (C₂ alkylenyl)-(cyclopropylene-C₂ alkyl). In some embodiments, Lipids of the Disclosure have a structure of Formula (X), wherein each R″ is independently (C₁ alkylenyl)-(cyclopropylene-C₆ alkyl). In some embodiments, Lipids of the Disclosure have a structure of Formula (X), wherein each R^{ww} is independently (C₂ alkylenyl)-(cyclopropylene-C₂ alkyl).

In some embodiments, Lipids of the Disclosure have a structure of Formula (X), wherein each R^{ww} is C₄ alkyl. In some embodiments, Lipids of the Disclosure have a structure of Formula (X), wherein each R^{ww} is C₅ alkyl. In some embodiments, Lipids of the Disclosure have a structure of Formula (X), wherein each R^{ww} is C₆ alkyl. In some embodiments, Lipids of the Disclosure have a structure of Formula (X), wherein each R^{ww} is C₇ alkyl. In some embodiments, Lipids of the Disclosure have a structure of Formula (X), wherein each R^{ww} is C₈ alkyl. In some embodiments, Lipids of the Disclosure have a structure of Formula (X), wherein each R^{ww} is C₉ alkyl. In some embodiments, Lipids of the Disclosure have a structure of Formula (X), wherein each R^{ww} is C₁₀ alkyl. In some embodiments, Lipids of the Disclosure have a structure of Formula (X), wherein each R^{ww} is C₁₁ alkyl. In some embodiments, Lipids of the Disclosure have a structure of Formula (X), wherein each R^{ww} is C₁₂ alkyl. In some embodiments, Lipids of the Disclosure have a structure of Formula (X), wherein each R^{ww} is C₁₃ alkyl. In some embodiments, Lipids of the Disclosure have a structure of Formula (X), wherein each R^{ww} is C₁₄ alkyl.

In some embodiments, Lipids of the Disclosure have a structure of Formula (X), wherein each R^{ww} is C₉ alkenyl. In some embodiments, Lipids of the Disclosure have a structure of Formula (X), wherein each R^{ww} is C₁₀ alkenyl. In some embodiments, Lipids of the Disclosure have a structure of Formula (X), wherein each R^{ww} is C₁₁ alkenyl. In some embodiments, Lipids of the Disclosure have a structure of Formula (X), wherein each R^{ww} is C₁₂ alkenyl.

In some embodiments, Lipids of the Disclosure have a structure of Formula (X), wherein each R^{ww} is C₈ alkenyl comprising at least two double bonds. In some embodiments, Lipids of the Disclosure have a structure of Formula (X), wherein each R^{ww} is C₉ alkenyl comprising at least two double bonds. In some embodiments, Lipids of the Disclosure have a structure of Formula (X), wherein each R^{ww} is C₁₀ alkenyl comprising at least two double bonds. In some embodiments, Lipids of the Disclosure have a structure of Formula (X), wherein each R^{ww} is C₁₁ alkenyl comprising at least two double bonds. In some embodiments, Lipids of the Disclosure have a structure of Formula (X), wherein each R^{ww} is C₁₂ alkenyl comprising at least two double bonds. In some embodiments, Lipids of the Disclosure have a structure of Formula (X), wherein each R^{ww} is C₁₃ alkenyl comprising at least two double bonds. In some embodiments, Lipids of the Disclosure have a structure of Formula (X), wherein each R^{ww} is C₁₄ alkenyl comprising at least two double bonds.

In some embodiments, Lipids of the Disclosure have a structure of Formula (X), wherein each R^{ww} is C₉ alkyl, wherein one —(CH₂)₂— of the C₉ alkyl is replaced with C₂-C₆ cycloalkylenyl. In some embodiments, Lipids of the Disclosure have a structure of Formula (X), wherein each R^{ww} is C₉ alkyl, wherein one —(CH₂)₂— of the C₉ alkyl is replaced with cyclopropylene. In some embodiments, Lipids of the Disclosure have a structure of Formula (X), wherein each R^{ww} is C₉ alkyl, wherein two —(CH₂)₂— of the C₉ alkyl are replaced with C₂-C₆ cycloalkylenyl. In some embodiments, Lipids of the Disclosure have a structure of Formula (X), wherein each R^{ww} is C₉ alkyl, wherein two —(CH₂)₂— of the C₉ alkyl are replaced with cyclopropylene.

In some embodiments, Lipids of the Disclosure have a structure of Formula (X), wherein each R^{ww} is linear C₄ alkyl. In some embodiments, Lipids of the Disclosure have a structure of Formula (X), wherein each R^{ww} is linear C₅ alkyl. In some embodiments, Lipids of the Disclosure have a structure of Formula (X), wherein each R^{ww} is linear C₆ alkyl. In some embodiments, Lipids of the Disclosure have a structure of Formula (X), wherein each R^{ww} is linear C₇ alkyl. In some embodiments, Lipids of the Disclosure have a structure of Formula (X), wherein each R^{ww} is linear C₈ alkyl. In some embodiments, Lipids of the Disclosure have a structure of Formula (X), wherein each R^{ww} is linear C9 alkyl. In some embodiments, Lipids of the Disclosure have a structure of Formula (X), wherein each R^{ww} is linear C₁₀ alkyl. In some embodiments, Lipids of the Disclosure have a structure of Formula (X), wherein each R^{ww} is linear C₁₁ alkyl. In some embodiments, Lipids of the Disclosure have a structure of Formula (X), wherein each R^{ww} is linear C₁₂ alkyl. In some embodiments, Lipids of the Disclosure have a structure of Formula (X), wherein each R^{ww} is linear C₁₃ alkyl. In some embodiments, Lipids of the Disclosure have a structure of Formula (X), wherein each R^{ww} is linear C₁₄ alkyl.

In some embodiments, Lipids of the Disclosure have a structure of Formula (X), wherein each R^{ww} is branched C₈ alkenyl. In some embodiments, Lipids of the Disclosure have a structure of Formula (X), wherein each R^{ww} is branched C_{q} alkenyl. In some embodiments, Lipids of the Disclosure have a structure of Formula (X), wherein each R^{ww} is branched C₁₀ alkenyl. In some embodiments, Lipids of the Disclosure have a structure of Formula (X), wherein each R^{ww} is branched C₁₁ alkenyl. In some embodiments, Lipids of the Disclosure have a structure of Formula (X), wherein each R^{ww} is branched C₁₂ alkenyl.

In some embodiments, Lipids of the Disclosure have a structure of Formula (X), wherein each cc is independently selected from 3 to 7. In some embodiments, Lipids of the Disclosure have a structure of Formula (X), wherein each cc is 3. In some embodiments, Lipids of the Disclosure have a structure of Formula (X), wherein each cc is 4. In some embodiments, Lipids of the Disclosure have a structure of Formula (X), wherein each cc is 5. In some embodiments, Lipids of the Disclosure have a structure of Formula (X), wherein each cc is 6. In some embodiments, Lipids of the Disclosure have a structure of Formula (X), wherein each cc is 7. In some embodiments, Lipids of the Disclosure have a structure of Formula (X), wherein each cc is 8. In some embodiments, Lipids of the Disclosure have a structure of Formula (X), wherein each cc is 9.

In some embodiments, Lipids of the Disclosure have a structure of Formula (X), wherein each dd is independently selected from 1 to 4. In some embodiments, Lipids of the Disclosure have a structure of Formula (X), wherein each dd is 1. In some embodiments, Lipids of the Disclosure have a structure of Formula (X), wherein each dd is 2. In some embodiments, Lipids of the Disclosure have a structure of Formula (X), wherein each dd is 3. In some embodiments, Lipids of the Disclosure have a structure of Formula (X), wherein each dd is 4.

In some embodiments, Lipids of the Disclosure have a structure of Formula (X), wherein ee is 1.

In some embodiments, Lipids of the Disclosure have a structure of Formula (X), wherein ee is 0.

### Formula (X-A)

In some embodiments, Lipids of the Disclosure have a structure of Formula (X), wherein the Lipids of the Disclosure have a structure of Formula (X-A):

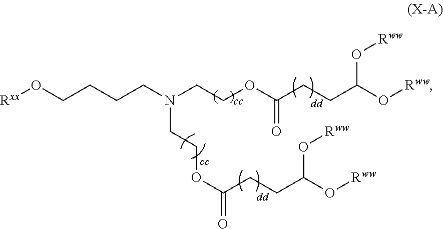

or a pharmaceutically acceptable salt thereof, wherein
each cc is independently selected from 3 to 7;
   each dd is independently selected from 1 to 4;
   R^{ww} is selected from hydrogen and optionally substituted C₁-C₆ alkyl; and
   each R^{ww} is independently selected from the group consisting of C₄-C₁₄ alkyl or (linear or branched C₃-C₅ alkylenyl)-(branched C₅-C₇alkenyl).

In some embodiments, Lipids of the Disclosure have a structure of Formula (X-A), wherein R^{xx} is hydrogen. In some embodiments, Lipids of the Disclosure have a structure of Formula (X-A), wherein R^{xx} is C₁ alkyl. In some embodiments, Lipids of the Disclosure have a structure of Formula (X-A), wherein R^{xx} is C₂ alkyl. In some embodiments, Lipids of the Disclosure have a structure of Formula (X-A), wherein R^{xx} is C₃ alkyl. In some embodiments, Lipids of the Disclosure have a structure of Formula (X-A), wherein R^{xx} is C₄ alkyl. In some embodiments, Lipids of the Disclosure have a structure of Formula (X-A), wherein R^{xx} is C₅ alkyl. In some embodiments, Lipids of the Disclosure have a structure of Formula (X-A), wherein R^{xx} is C₆ alkyl.

In some embodiments, Lipids of the Disclosure have a structure of Formula (X-A), wherein each cc is 4, 5, 6, or 7. In some embodiments, Lipids of the Disclosure have a structure of Formula (X-A), wherein each cc is 3. In some embodiments, Lipids of the Disclosure have a structure of Formula (X-A), wherein each cc is 4. In some embodiments, Lipids of the Disclosure have a structure of Formula (X-A), wherein each cc is 5. In some embodiments, Lipids of the Disclosure have a structure of Formula (X-A), wherein each cc is 6. In some embodiments, Lipids of the Disclosure have a structure of Formula (X-A), wherein each cc is 7.

In some embodiments, Lipids of the Disclosure have a structure of Formula (X-A), wherein each dd is 1 or 3. In some embodiments, Lipids of the Disclosure have a structure of Formula (X-A), wherein each dd is 1. In some embodiments, Lipids of the Disclosure have a structure of Formula (X-A), wherein each dd is 2. In some embodiments, Lipids of the Disclosure have a structure of Formula (X-A), wherein each dd is 3. In some embodiments, Lipids of the Disclosure have a structure of Formula (X-A), wherein each dd is 4.

In some embodiments, Lipids of the Disclosure have a structure of Formula (X-A), wherein each R^{ww} is C₄-C₁₄ alkyl. In some embodiments, Lipids of the Disclosure have a structure of Formula (X-A), wherein each R^{ww} is C₄ alkyl. In some embodiments, Lipids of the Disclosure have a structure of Formula (X-A), wherein each R^{ww} is C₅ alkyl. In some embodiments, Lipids of the Disclosure have a structure of Formula (X-A), wherein each R^{ww} is C₆ alkyl. In some embodiments, Lipids of the Disclosure have a structure of Formula (X-A), wherein each R^{ww} is C₇ alkyl. In some embodiments, Lipids of the Disclosure have a structure of Formula (X-A), wherein each R^{ww} is C₈ alkyl. In some embodiments, Lipids of the Disclosure have a structure of Formula (X-A), wherein each R^{ww} is C₉ alkyl. In some embodiments, Lipids of the Disclosure have a structure of Formula (X-A), wherein each R^{ww} is C₁₀ alkyl. In some embodiments, Lipids of the Disclosure have a structure of Formula (X-A), wherein each R^{ww} is C₁₁ alkyl. In some embodiments, Lipids of the Disclosure have a structure of Formula (X-A), wherein each R^{ww} is C₁₂ alkyl. In some embodiments, Lipids of the Disclosure have a structure of Formula (X-A), wherein each R^{ww} is C₁₃ alkyl. In some embodiments, Lipids of the Disclosure have a structure of Formula (X-A), wherein each R^{ww} is C₁₄ alkyl.

In some embodiments, Lipids of the Disclosure have a structure of Formula (X-A), wherein each R^{ww} is (linear or branched C₃-C₅ alkylenyl)-(branched C₅-C₇alkenyl), e.g., (branched C₅ alkylenyl)-(branched C₅alkenyl), e.g.,

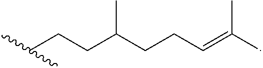

In some embodiments, Lipids of the Disclosure comprise an acyclic core. In some embodiments, Lipids of the Disclosure are selected from any lipid in Table (I) below or a pharmaceutically acceptable salt thereof:

TABLE (I)
Non-Limiting Examples of Ionizable Lipids with an Acyclic Core

| | Com- |
|---|---|
| | pound |
| Structure | No. |
| 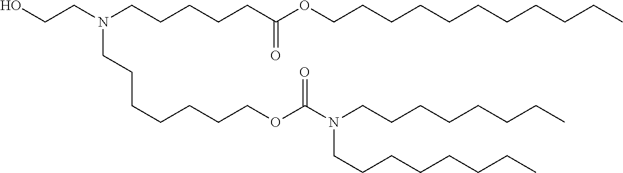 [SMILES: CCCCCCCCCCCOC(=O)CCCCCN(CCO)CCCCCCCOC(=O)N(CCCCCCCC)CCCCCCCC] | 1 |
| 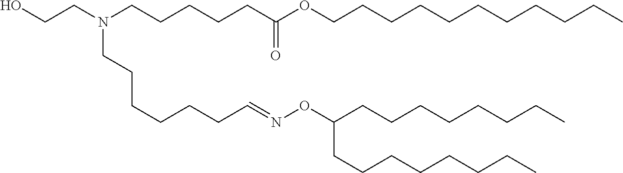 [SMILES: CCCCCCCCCCCOC(=O)CCCCCN(CCO)CCCCCC/C=N/OC(CCCCCCCC)CCCCCCCC] | 2 |
| 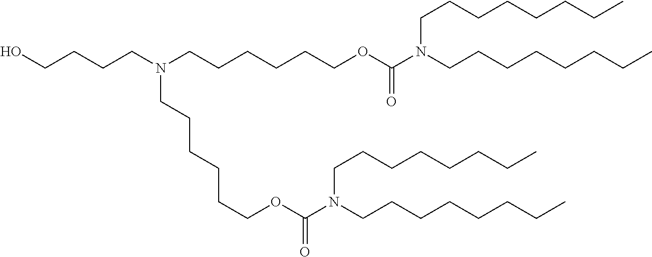 [SMILES: CCCCCCCCN(CCCCCCCC)C(=O)OCCCCCCN(CCCCO)CCCCCCOC(=O)N(CCCCCCCC)CCCCCCCC] | 3 |
| 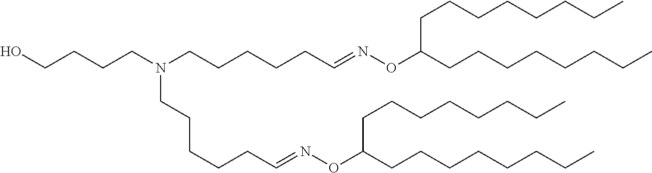 [SMILES: CCCCCCCCC(CCCCCCCC)O/N=C/CCCCCN(CCCCO)CCCCC/C=N/OC(CCCCCCCC)CCCCCCCC] | 4 |
| 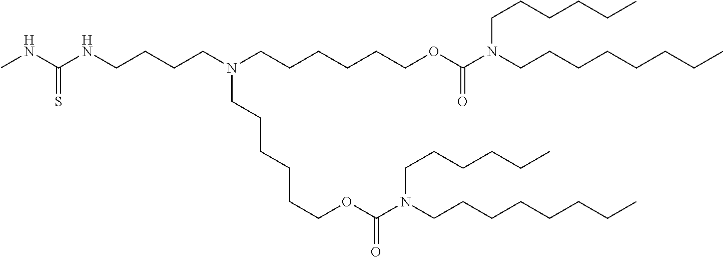 [SMILES: CCCCCCCCN(CCCCCC)C(=O)OCCCCCCN(CCCCCCOC(=O)N(CCCCCC)CCCCCCCC)CCCCNC(=S)NC] | 5 |
| 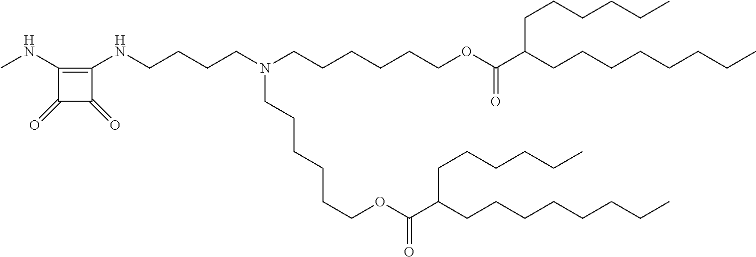 [SMILES: CCCCCCCCC(CCCCCC)C(=O)OCCCCCCN(CCCCCCOC(=O)C(CCCCCC)CCCCCCCC)CCCCNc1c(NC)c(=O)c1=O] | 6 |
| 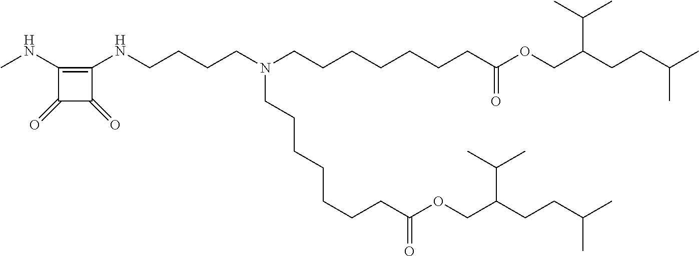 [SMILES: CNc1c(NCCCCN(CCCCCCCC(=O)OCC(CCC(C)C)C(C)C)CCCCCCCC(=O)OCC(CCC(C)C)C(C)C)c(=O)c1=O] | 7 |
| 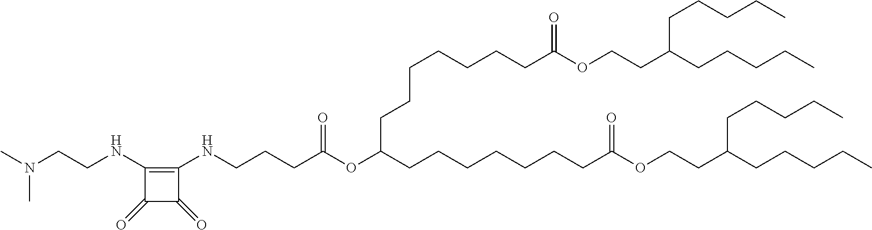 [SMILES: CCCCCC(CCCCC)CCOC(=O)CCCCCCCC(CCCCCCCC(=O)OCCC(CCCCC)CCCCC)OC(=O)CCCNc1c(NCCN(C)C)c(=O)c1=O] | 8 |
| 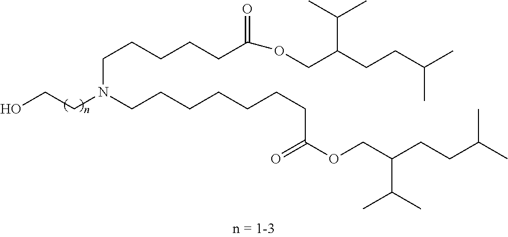 | 9 |
| 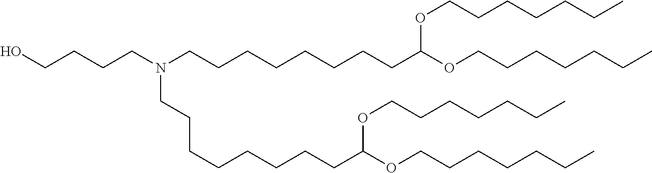 [SMILES: CCCCCCCOC(CCCCCCCCN(CCCCO)CCCCCCCCC(OCCCCCCC)OCCCCCCC)OCCCCCCC] | 10 |
| 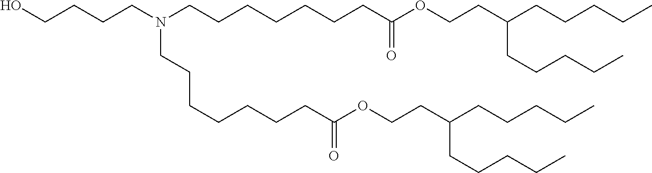 [SMILES: CCCCCC(CCCCC)CCOC(=O)CCCCCCCN(CCCCO)CCCCCCCC(=O)OCCC(CCCCC)CCCCC] | 11 |
| 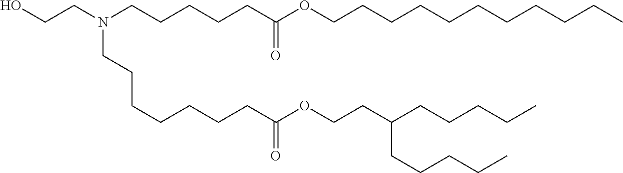 [SMILES: CCCCCCCCCCCOC(=O)CCCCCN(CCO)CCCCCCCC(=O)OCCC(CCCCC)CCCCC] | 13 |
| 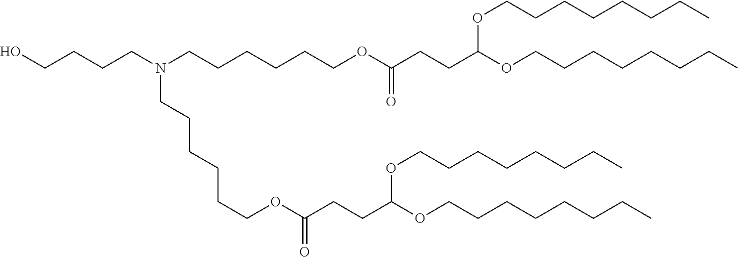 [SMILES: CCCCCCCCOC(CCC(=O)OCCCCCCN(CCCCO)CCCCCCOC(=O)CCC(OCCCCCCCC)OCCCCCCCC)OCCCCCCCC] | 14 |
| 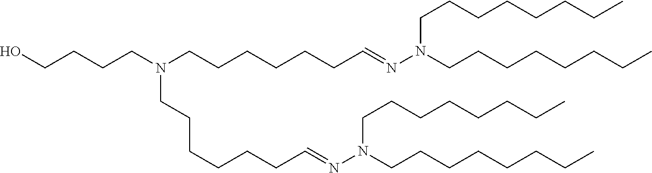 [SMILES: CCCCCCCCN(CCCCCCCC)/N=C/CCCCCCN(CCCCO)CCCCCC/C=N/N(CCCCCCCC)CCCCCCCC] | 15 |
| 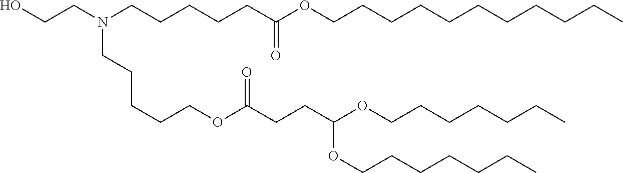 [SMILES: CCCCCCCCCCCOC(=O)CCCCCN(CCO)CCCCCOC(=O)CCC(OCCCCCCC)OCCCCCCC] | 16 |
| 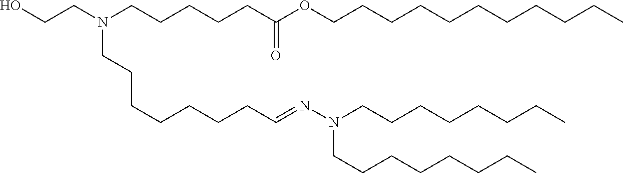 [SMILES: CCCCCCCCCCCOC(=O)CCCCCN(CCO)CCCCCCC/C=N/N(CCCCCCCC)CCCCCCCC] | 17 |
| 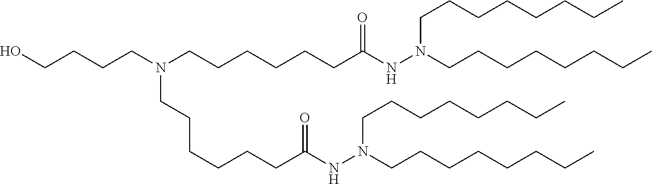 [SMILES: CCCCCCCCN(CCCCCCCC)NC(=O)CCCCCCN(CCCCO)CCCCCCC(=O)NN(CCCCCCCC)CCCCCCCC] | 18 |
| 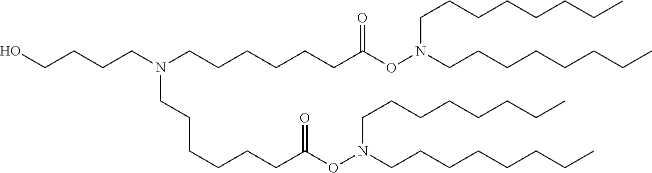 [SMILES: CCCCCCCCN(CCCCCCCC)OC(=O)CCCCCCN(CCCCO)CCCCCCC(=O)ON(CCCCCCCC)CCCCCCCC] | 19 |
| 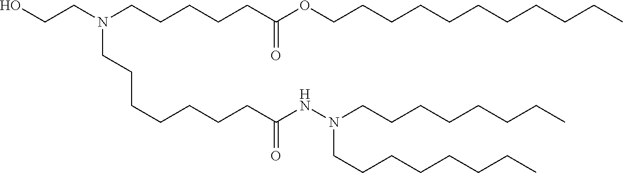 [SMILES: CCCCCCCCCCCOC(=O)CCCCCN(CCO)CCCCCCCC(=O)NN(CCCCCCCC)CCCCCCCC] | 20 |
| 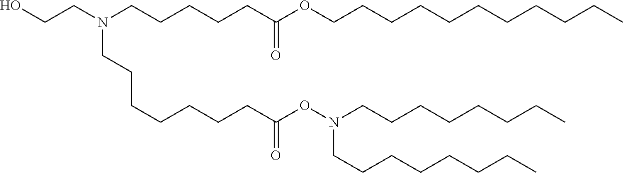 [SMILES: CCCCCCCCCCCOC(=O)CCCCCN(CCO)CCCCCCCC(=O)ON(CCCCCCCC)CCCCCCCC] | 21 |
| 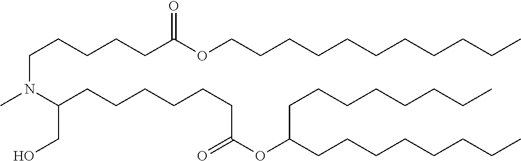 [SMILES: CCCCCCCCCCCOC(=O)CCCCCN(C)C(CO)CCCCCCC(=O)OC(CCCCCCCC)CCCCCCCC] | 22 |
| 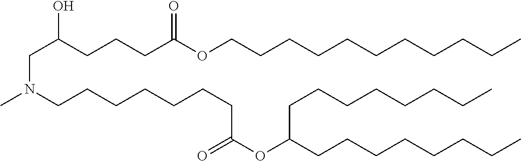 [SMILES: CCCCCCCCCCCOC(=O)CCCC(O)CN(C)CCCCCCCC(=O)OC(CCCCCCCC)CCCCCCCC] | 23 |
| 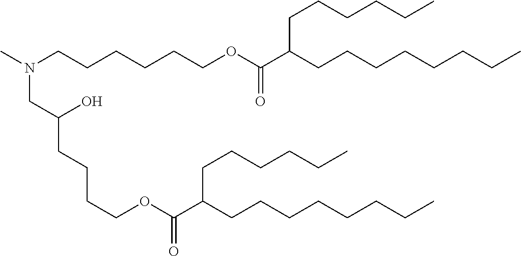 [SMILES: CCCCCCCCC(CCCCCC)C(=O)OCCCCCCN(C)CC(O)CCCCOC(=O)C(CCCCCC)CCCCCCCC] | 24 |
| 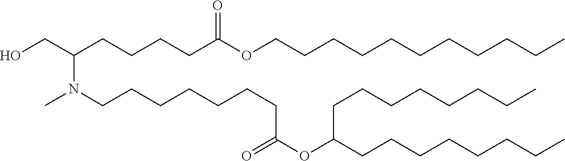 [SMILES: CCCCCCCCCCCOC(=O)CCCCC(CO)N(C)CCCCCCCC(=O)OC(CCCCCCCC)CCCCCCCC] | 25 |
| 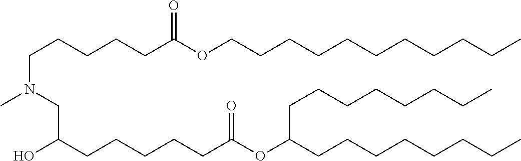 [SMILES: CCCCCCCCCCCOC(=O)CCCCCN(C)CC(O)CCCCCC(=O)OC(CCCCCCCC)CCCCCCCC] | 26 |
| 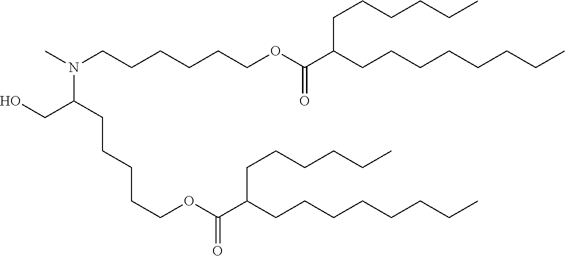 [SMILES: CCCCCCCCC(CCCCCC)C(=O)OCCCCCCN(C)C(CO)CCCCCOC(=O)C(CCCCCC)CCCCCCCC] | 27 |
| 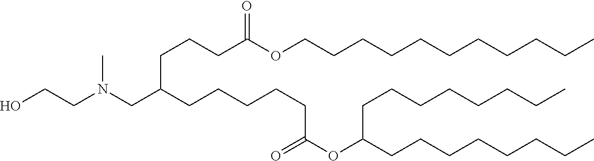 [SMILES: CCCCCCCCCCCOC(=O)CCCC(CCCCCC(=O)OC(CCCCCCCC)CCCCCCCC)CN(C)CCO] | 28 |
| 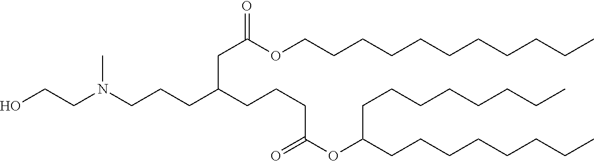 [SMILES: CCCCCCCCCCCOC(=O)CC(CCCC(=O)OC(CCCCCCCC)CCCCCCCC)CCCN(C)CCO] | 29 |
| 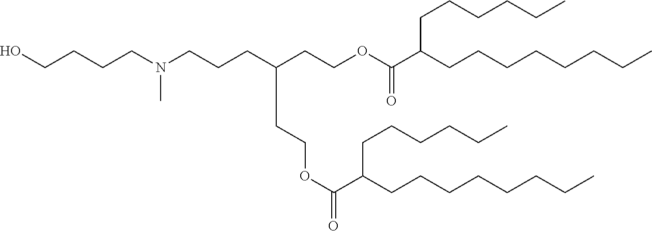 [SMILES: CCCCCCCCC(CCCCCC)C(=O)OCCC(CCCN(C)CCCCO)CCOC(=O)C(CCCCCC)CCCCCCCC] | 30 |
| 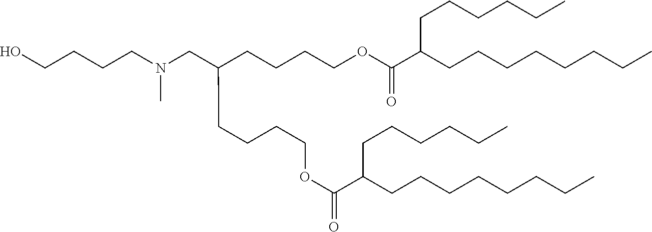 [SMILES: CCCCCCCCC(CCCCCC)C(=O)OCCCCC(CCCCOC(=O)C(CCCCCC)CCCCCCCC)CN(C)CCCCO] | 31 |
| 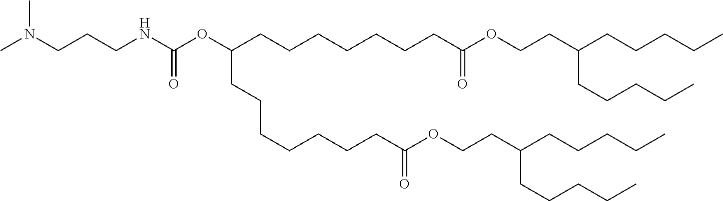 [SMILES: CCCCCC(CCCCC)CCOC(=O)CCCCCCCC(CCCCCCCC(=O)OCCC(CCCCC)CCCCC)OC(=O)NCCCN(C)C] | 32 |
| 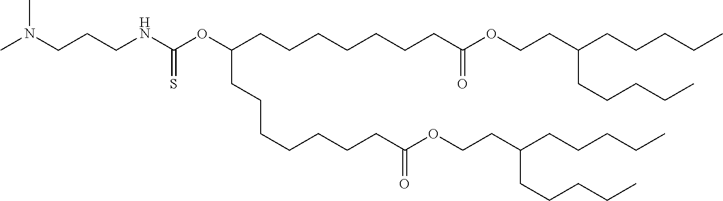 [SMILES: CCCCCC(CCCCC)CCOC(=O)CCCCCCCC(CCCCCCCC(=O)OCCC(CCCCC)CCCCC)OC(=S)NCCCN(C)C] | 33 |
| 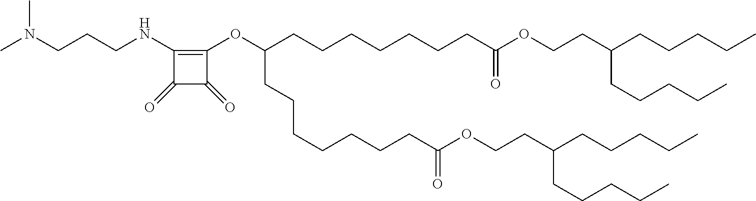 [SMILES: CCCCCC(CCCCC)CCOC(=O)CCCCCCCC(CCCCCCCC(=O)OCCC(CCCCC)CCCCC)Oc1c(NCCCN(C)C)c(=O)c1=O] | 34 |
| 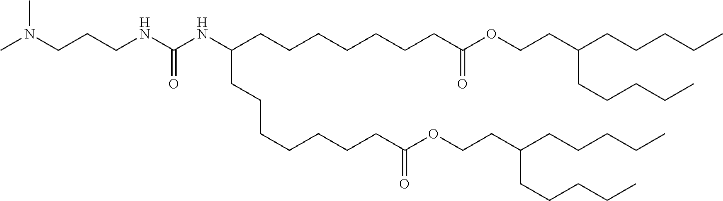 [SMILES: CCCCCC(CCCCC)CCOC(=O)CCCCCCCC(CCCCCCCC(=O)OCCC(CCCCC)CCCCC)NC(=O)NCCCN(C)C] | 35 |
| 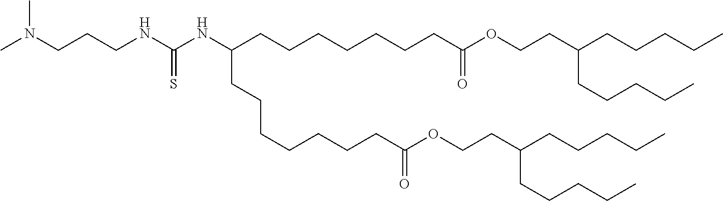 [SMILES: CCCCCC(CCCCC)CCOC(=O)CCCCCCCC(CCCCCCCC(=O)OCCC(CCCCC)CCCCC)NC(=S)NCCCN(C)C] | 36 |
| 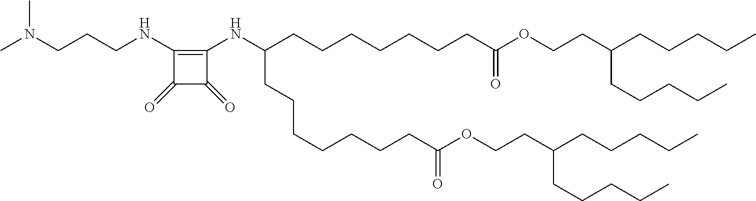 [SMILES: CCCCCC(CCCCC)CCOC(=O)CCCCCCCC(CCCCCCCC(=O)OCCC(CCCCC)CCCCC)Nc1c(NCCCN(C)C)c(=O)c1=O] | 37 |
| 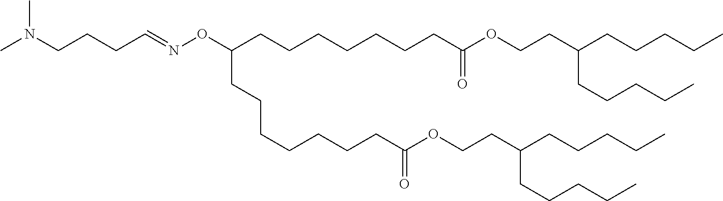 [SMILES: CCCCCC(CCCCC)CCOC(=O)CCCCCCCC(CCCCCCCC(=O)OCCC(CCCCC)CCCCC)O/N=C/CCCN(C)C] | 38 |
| 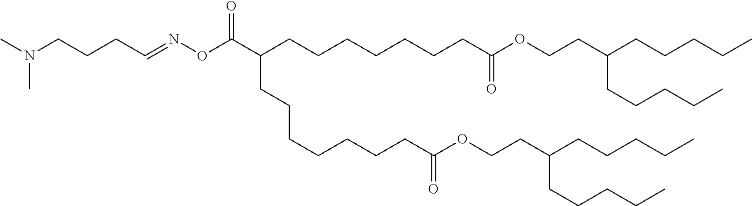 [SMILES: CCCCCC(CCCCC)CCOC(=O)CCCCCCCC(CCCCCCCC(=O)OCCC(CCCCC)CCCCC)C(=O)O/N=C/CCCN(C)C] | 39 |
| 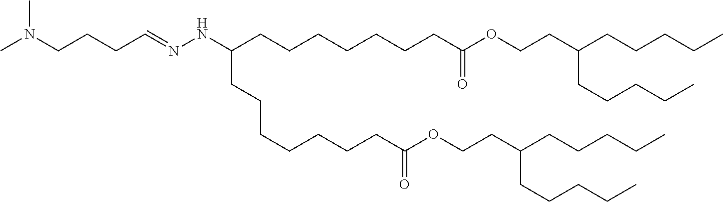 [SMILES: CCCCCC(CCCCC)CCOC(=O)CCCCCCCC(CCCCCCCC(=O)OCCC(CCCCC)CCCCC)N/N=C/CCCN(C)C] | 40 |
| 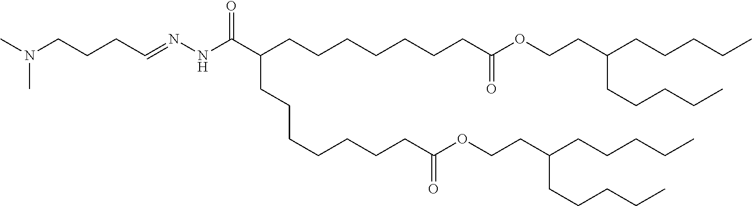 [SMILES: CCCCCC(CCCCC)CCOC(=O)CCCCCCCC(CCCCCCCC(=O)OCCC(CCCCC)CCCCC)C(=O)N/N=C/CCCN(C)C] | 41 |
| 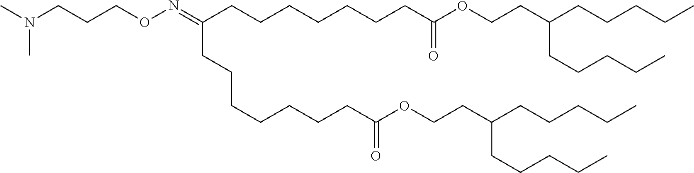 [SMILES: CCCCCC(CCCCC)CCOC(=O)CCCCCCCC(CCCCCCCC(=O)OCCC(CCCCC)CCCCC)=NOCCCN(C)C] | 42 |
| 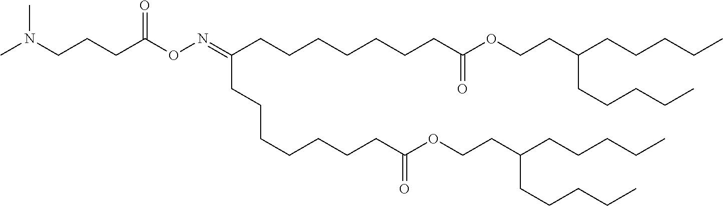 [SMILES: CCCCCC(CCCCC)CCOC(=O)CCCCCCCC(CCCCCCCC(=O)OCCC(CCCCC)CCCCC)=NOC(=O)CCCN(C)C] | 43 |
| 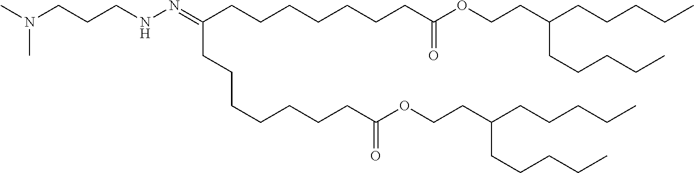 [SMILES: CCCCCC(CCCCC)CCOC(=O)CCCCCCCC(CCCCCCCC(=O)OCCC(CCCCC)CCCCC)=NNCCCN(C)C] | 44 |
| 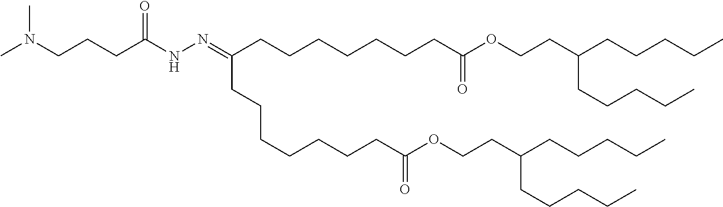 [SMILES: CCCCCC(CCCCC)CCOC(=O)CCCCCCCC(CCCCCCCC(=O)OCCC(CCCCC)CCCCC)=NNC(=O)CCCN(C)C] | 45 |
| 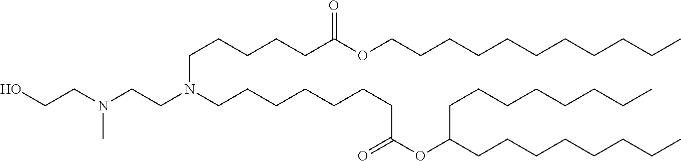 [SMILES: CCCCCCCCCCCOC(=O)CCCCCN(CCCCCCCC(=O)OC(CCCCCCCC)CCCCCCCC)CCN(C)CCO] | 46 |
| 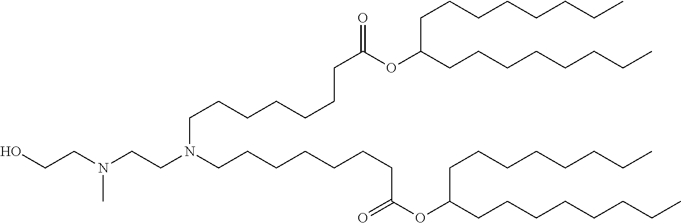 [SMILES: CCCCCCCCC(CCCCCCCC)OC(=O)CCCCCCCN(CCCCCCCC(=O)OC(CCCCCCCC)CCCCCCCC)CCN(C)CCO] | 47 |
| 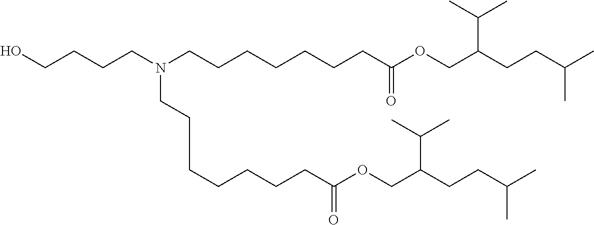 [SMILES: CC(C)CCC(COC(=O)CCCCCCCN(CCCCO)CCCCCCCC(=O)OCC(CCC(C)C)C(C)C)C(C)C] | 48 |
| 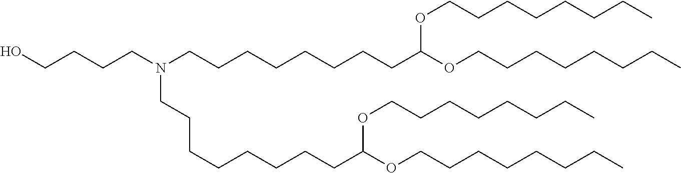 [SMILES: CCCCCCCCOC(CCCCCCCCN(CCCCO)CCCCCCCCC(OCCCCCCCC)OCCCCCCCC)OCCCCCCCC] | 49 |
| 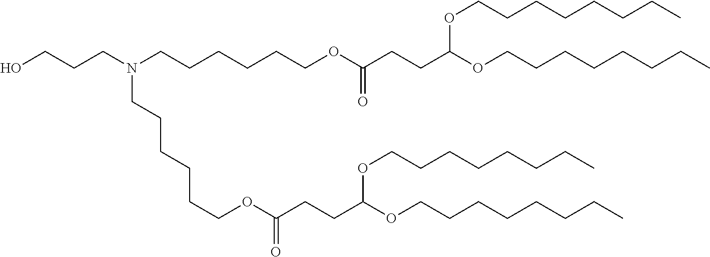 [SMILES: CCCCCCCCOC(CCC(=O)OCCCCCCN(CCCO)CCCCCCOC(=O)CCC(OCCCCCCCC)OCCCCCCCC)OCCCCCCCC] | 50 |
| 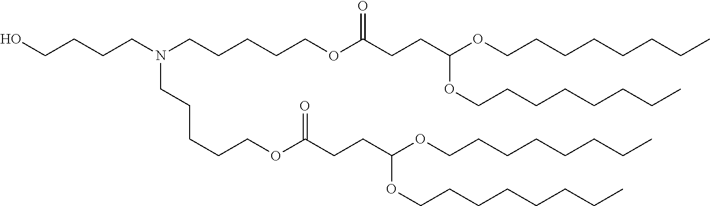 [SMILES: CCCCCCCCOC(CCC(=O)OCCCCCN(CCCCO)CCCCCOC(=O)CCC(OCCCCCCCC)OCCCCCCCC)OCCCCCCCC] | 51 |
| 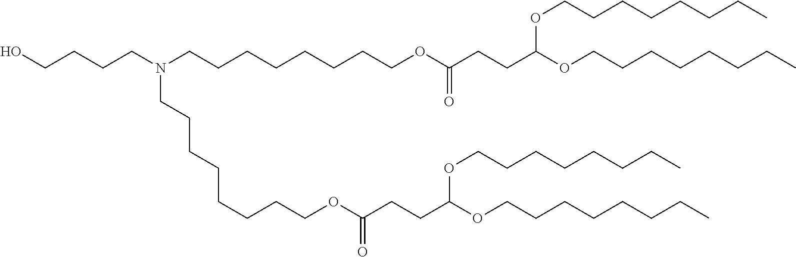 [SMILES: CCCCCCCCOC(CCC(=O)OCCCCCCCCN(CCCCO)CCCCCCCCOC(=O)CCC(OCCCCCCCC)OCCCCCCCC)OCCCCCCCC] | 52 |
| 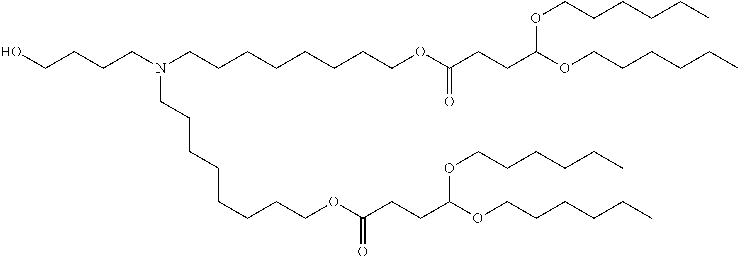 [SMILES: CCCCCCOC(CCC(=O)OCCCCCCCCN(CCCCO)CCCCCCCCOC(=O)CCC(OCCCCCC)OCCCCCC)OCCCCCC] | 53 |
| 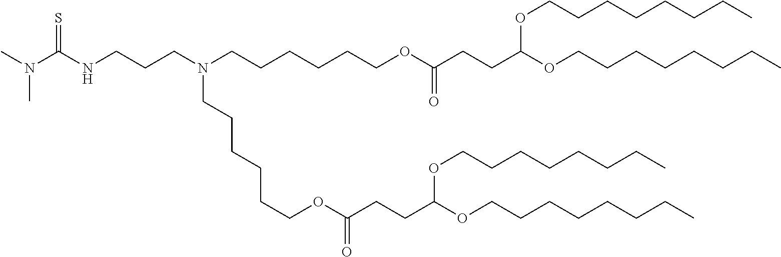 [SMILES: CCCCCCCCOC(CCC(=O)OCCCCCCN(CCCCCCOC(=O)CCC(OCCCCCCCC)OCCCCCCCC)CCCNC(=S)N(C)C)OCCCCCCCC] | 54 |
| 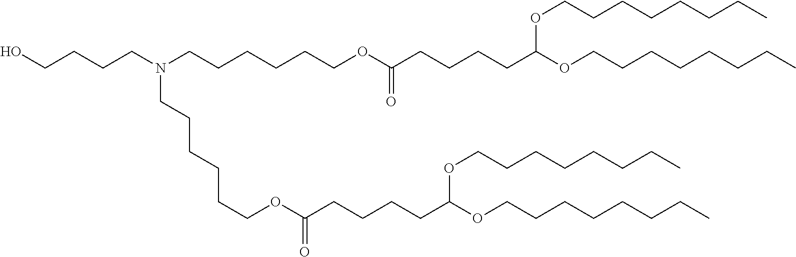 [SMILES: CCCCCCCCOC(CCCCC(=O)OCCCCCCN(CCCCO)CCCCCCOC(=O)CCCCC(OCCCCCCCC)OCCCCCCCC)OCCCCCCCC] | 55 |
| 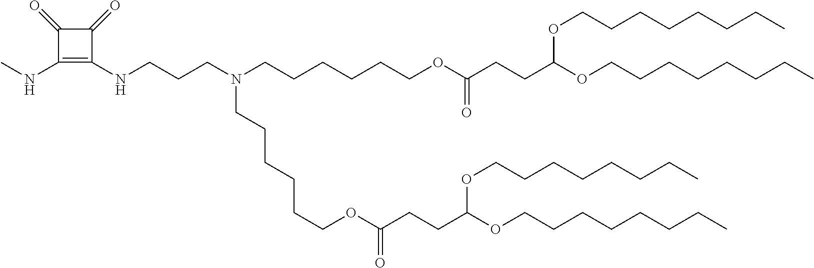 [SMILES: CCCCCCCCOC(CCC(=O)OCCCCCCN(CCCCCCOC(=O)CCC(OCCCCCCCC)OCCCCCCCC)CCCNc1c(NC)c(=O)c1=O)OCCCCCCCC] | 56 |
| 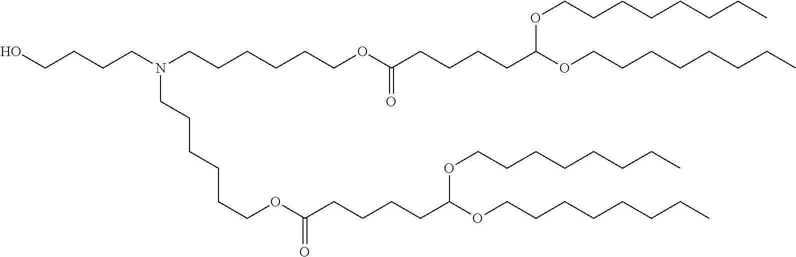 [SMILES: CCCCCCCCOC(CCCCC(=O)OCCCCCCN(CCCCO)CCCCCCOC(=O)CCCCC(OCCCCCCCC)OCCCCCCCC)OCCCCCCCC] | 57 |
| 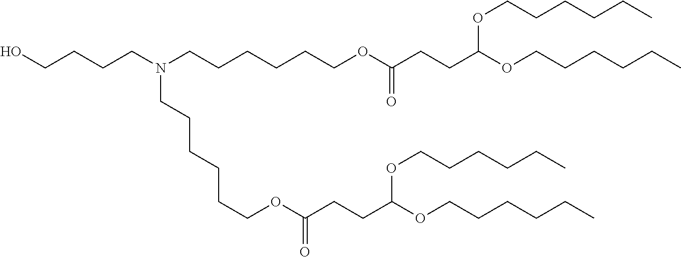 [SMILES: CCCCCCOC(CCC(=O)OCCCCCCN(CCCCO)CCCCCCOC(=O)CCC(OCCCCCC)OCCCCCC)OCCCCCC] | 58 |
| 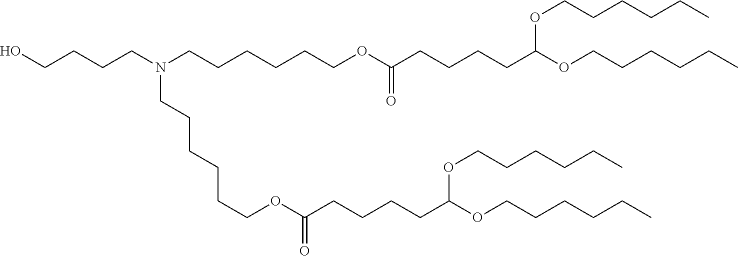 [SMILES: CCCCCCOC(CCCCC(=O)OCCCCCCN(CCCCO)CCCCCCOC(=O)CCCCC(OCCCCCC)OCCCCCC)OCCCCCC] | 59 |
| 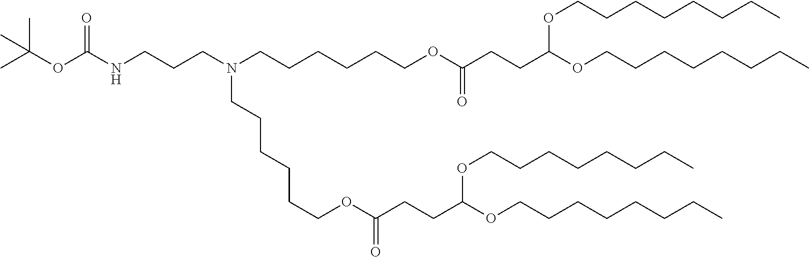 [SMILES: CCCCCCCCOC(CCC(=O)OCCCCCCN(CCCCCCOC(=O)CCC(OCCCCCCCC)OCCCCCCCC)CCCNC(=O)OC(C)(C)C)OCCCCCCCC] | 60 |
| 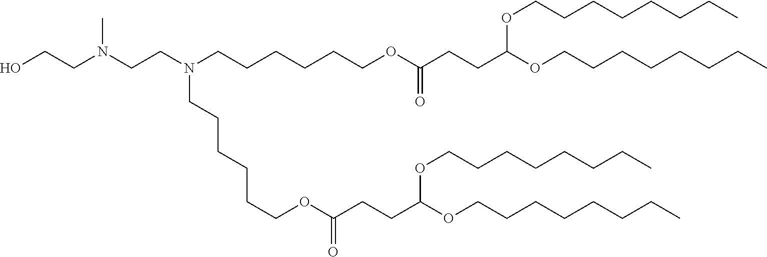 [SMILES: CCCCCCCCOC(CCC(=O)OCCCCCCN(CCCCCCOC(=O)CCC(OCCCCCCCC)OCCCCCCCC)CCN(C)CCO)OCCCCCCCC] | 61 |
| 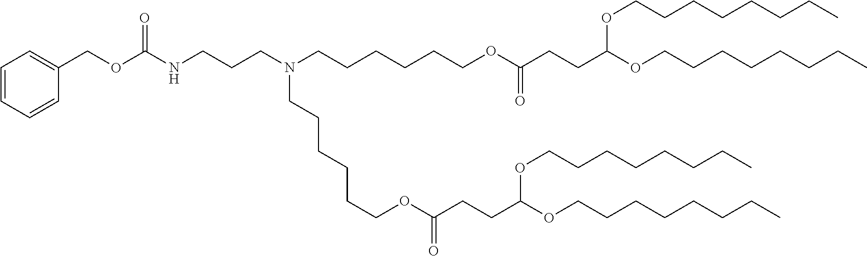 [SMILES: CCCCCCCCOC(CCC(=O)OCCCCCCN(CCCCCCOC(=O)CCC(OCCCCCCCC)OCCCCCCCC)CCCNC(=O)OCc1ccccc1)OCCCCCCCC] | 62 |
| 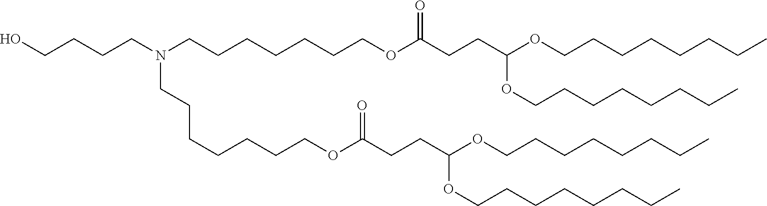 [SMILES: CCCCCCCCOC(CCC(=O)OCCCCCCCN(CCCCO)CCCCCCCOC(=O)CCC(OCCCCCCCC)OCCCCCCCC)OCCCCCCCC] | 64 |
| 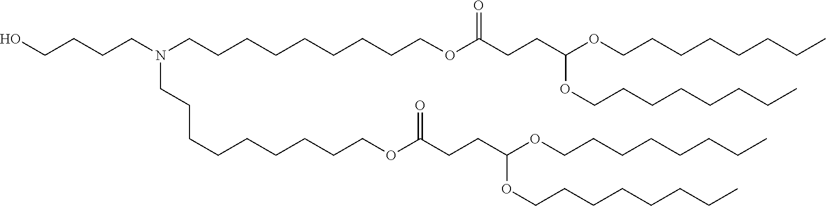 [SMILES: CCCCCCCCOC(CCC(=O)OCCCCCCCCCN(CCCCO)CCCCCCCCCOC(=O)CCC(OCCCCCCCC)OCCCCCCCC)OCCCCCCCC] | 65 |
| 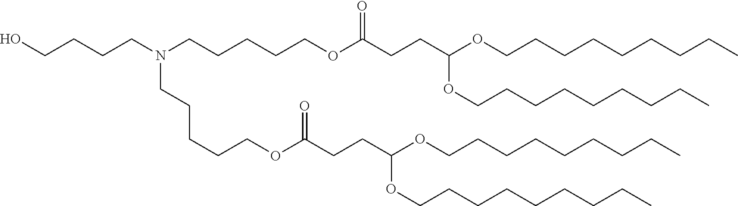 [SMILES: CCCCCCCCCOC(CCC(=O)OCCCCCN(CCCCO)CCCCCOC(=O)CCC(OCCCCCCCCC)OCCCCCCCCC)OCCCCCCCCC] | 66 |
| 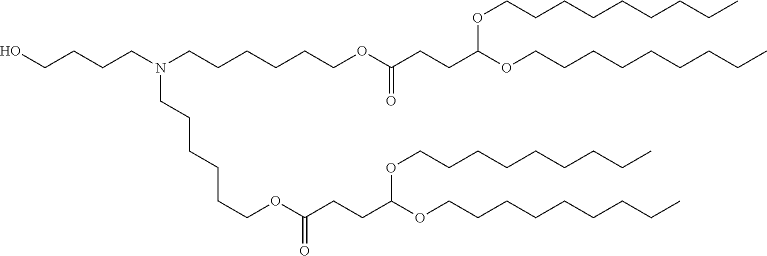 [SMILES: CCCCCCCCCOC(CCC(=O)OCCCCCCN(CCCCO)CCCCCCOC(=O)CCC(OCCCCCCCCC)OCCCCCCCCC)OCCCCCCCCC] | 67 |
| 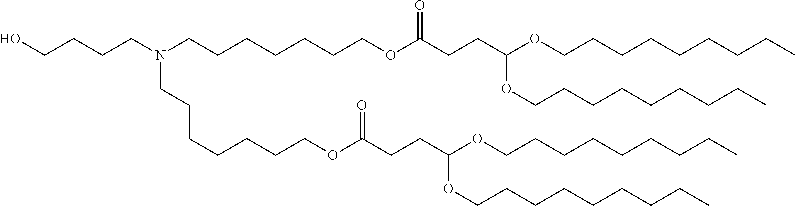 [SMILES: CCCCCCCCCOC(CCC(=O)OCCCCCCCN(CCCCO)CCCCCCCOC(=O)CCC(OCCCCCCCCC)OCCCCCCCCC)OCCCCCCCCC] | 68 |
| 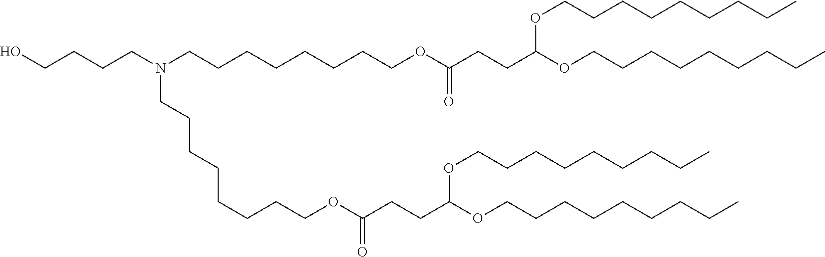 [SMILES: CCCCCCCCCOC(CCC(=O)OCCCCCCCCN(CCCCO)CCCCCCCCOC(=O)CCC(OCCCCCCCCC)OCCCCCCCCC)OCCCCCCCCC] | 69 |
| 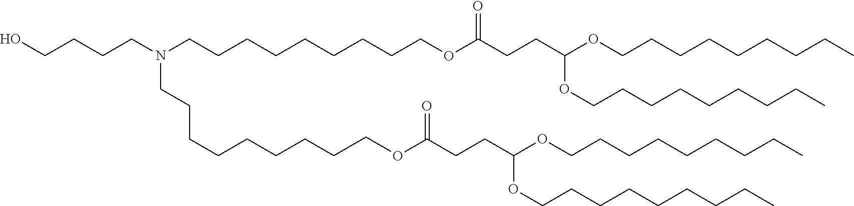 [SMILES: CCCCCCCCCOC(CCC(=O)OCCCCCCCCCN(CCCCO)CCCCCCCCCOC(=O)CCC(OCCCCCCCCC)OCCCCCCCCC)OCCCCCCCCC] | 70 |
| 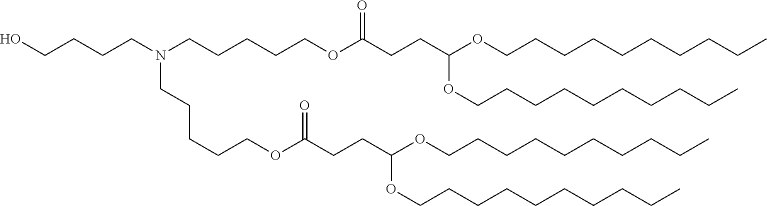 [SMILES: CCCCCCCCCCOC(CCC(=O)OCCCCCN(CCCCO)CCCCCOC(=O)CCC(OCCCCCCCCCC)OCCCCCCCCCC)OCCCCCCCCCC] | 71 |
| 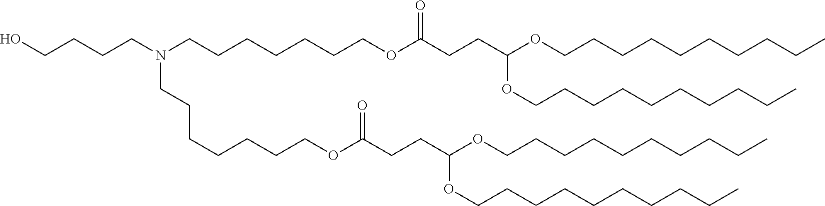 [SMILES: CCCCCCCCCCOC(CCC(=O)OCCCCCCCN(CCCCO)CCCCCCCOC(=O)CCC(OCCCCCCCCCC)OCCCCCCCCCC)OCCCCCCCCCC] | 72 |
| 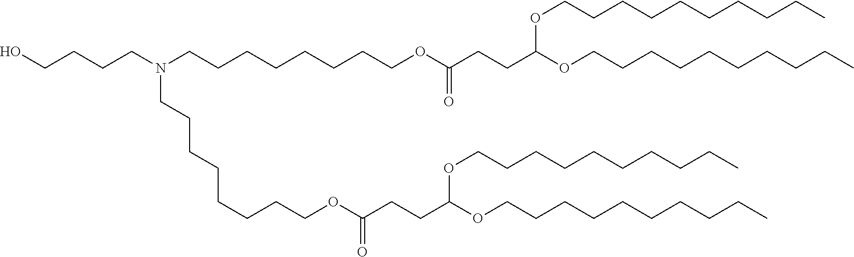 [SMILES: CCCCCCCCCCOC(CCC(=O)OCCCCCCCCN(CCCCO)CCCCCCCCOC(=O)CCC(OCCCCCCCCCC)OCCCCCCCCCC)OCCCCCCCCCC] | 73 |
| 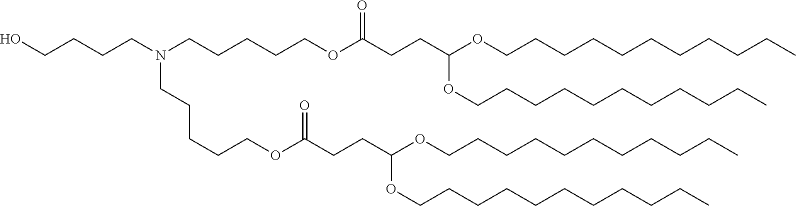 [SMILES: CCCCCCCCCCCOC(CCC(=O)OCCCCCN(CCCCO)CCCCCOC(=O)CCC(OCCCCCCCCCCC)OCCCCCCCCCCC)OCCCCCCCCCCC] | 74 |
| 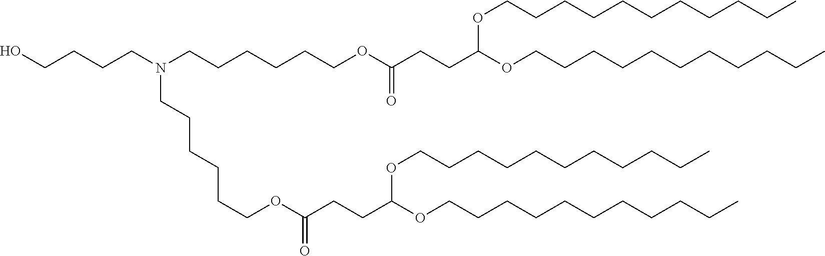 [SMILES: CCCCCCCCCCCOC(CCC(=O)OCCCCCCN(CCCCO)CCCCCCOC(=O)CCC(OCCCCCCCCCCC)OCCCCCCCCCCC)OCCCCCCCCCCC] | 75 |
| 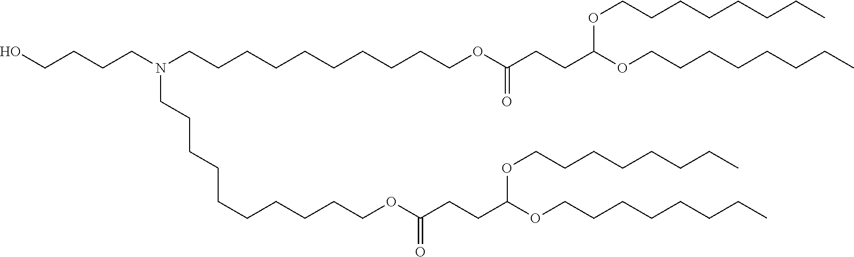 [SMILES: CCCCCCCCOC(CCC(=O)OCCCCCCCCCCN(CCCCO)CCCCCCCCCCOC(=O)CCC(OCCCCCCCC)OCCCCCCCC)OCCCCCCCC] | 76 |
| 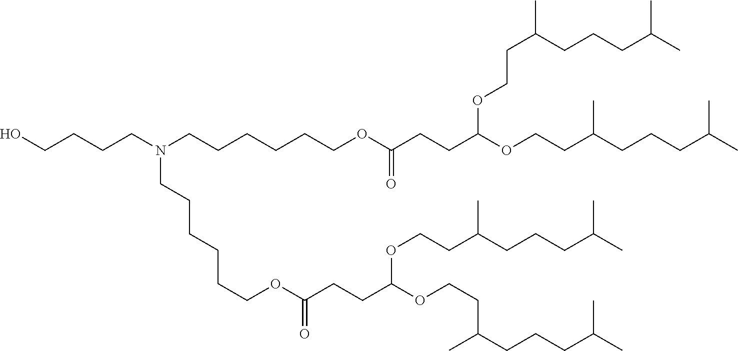 [SMILES: CC(C)CCCC(C)CCOC(CCC(=O)OCCCCCCN(CCCCO)CCCCCCOC(=O)CCC(OCCC(C)CCCC(C)C)OCCC(C)CCCC(C)C)OCCC(C)CCCC(C)C] | 77 |
| 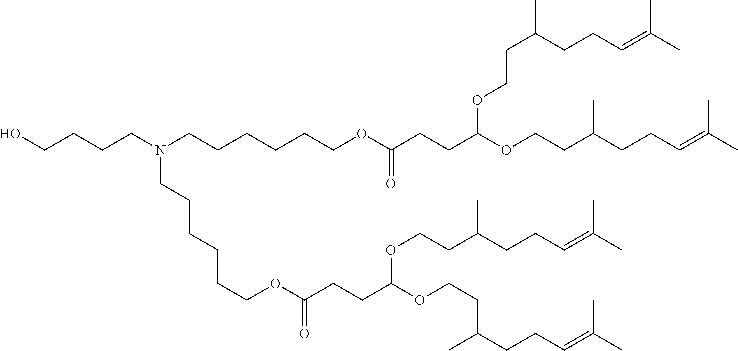 [SMILES: CC(C)=CCCC(C)CCOC(CCC(=O)OCCCCCCN(CCCCO)CCCCCCOC(=O)CCC(OCCC(C)CCC=C(C)C)OCCC(C)CCC=C(C)C)OCCC(C)CCC=C(C)C] | 78 |
| 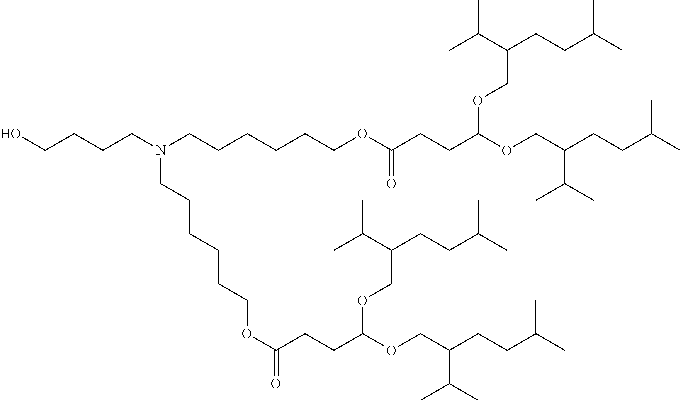 [SMILES: CC(C)CCC(COC(CCC(=O)OCCCCCCN(CCCCO)CCCCCCOC(=O)CCC(OCC(CCC(C)C)C(C)C)OCC(CCC(C)C)C(C)C)OCC(CCC(C)C)C(C)C)C(C)C] | 79 |
| 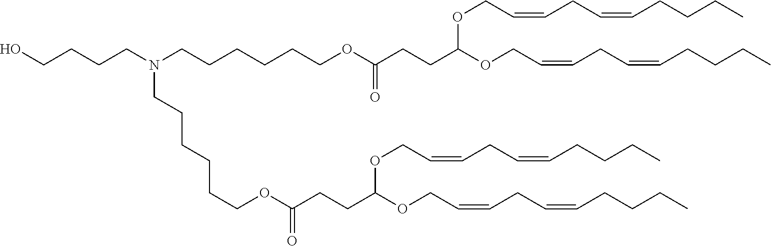 [SMILES: CCCC/C=C\C/C=C\COC(CCC(=O)OCCCCCCN(CCCCO)CCCCCCOC(=O)CCC(OC/C=C\C/C=C\CCCC)OC/C=C\C/C=C\CCCC)OC/C=C\C/C=C\CCCC] | 80 |
| 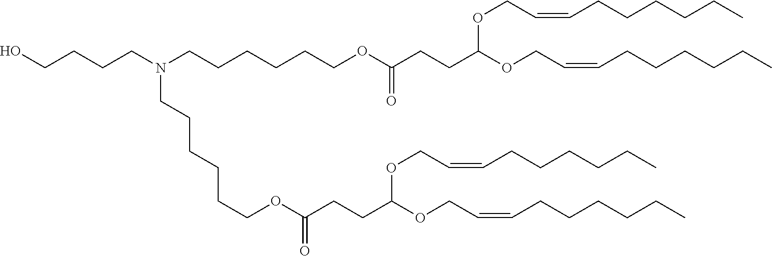 [SMILES: CCCCCC/C=C\COC(CCC(=O)OCCCCCCN(CCCCO)CCCCCCOC(=O)CCC(OC/C=C\CCCCCC)OC/C=C\CCCCCC)OC/C=C\CCCCCC] | 81 |
| 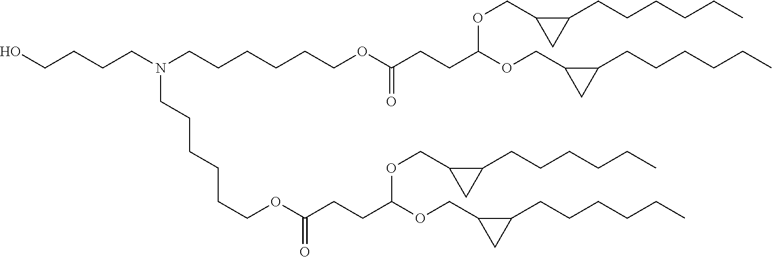 [SMILES: CCCCCCC1CC1COC(CCC(=O)OCCCCCCN(CCCCO)CCCCCCOC(=O)CCC(OCC1CC1CCCCCC)OCC1CC1CCCCCC)OCC1CC1CCCCCC] | 82 |
| 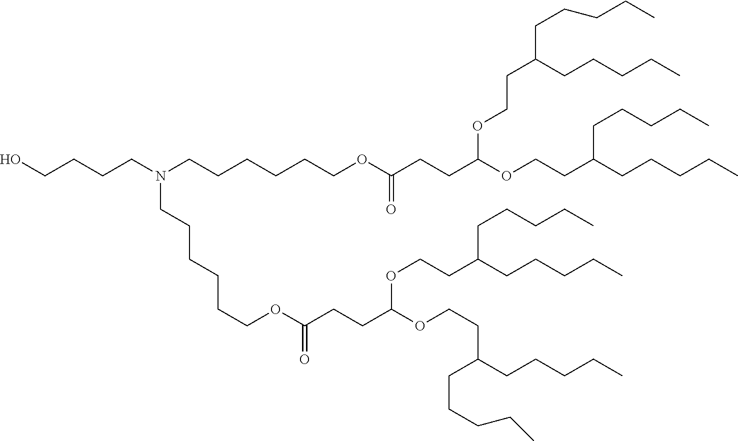 [SMILES: CCCCCC(CCCCC)CCOC(CCC(=O)OCCCCCCN(CCCCO)CCCCCCOC(=O)CCC(OCCC(CCCCC)CCCCC)OCCC(CCCCC)CCCCC)OCCC(CCCCC)CCCCC] | 83 |
| 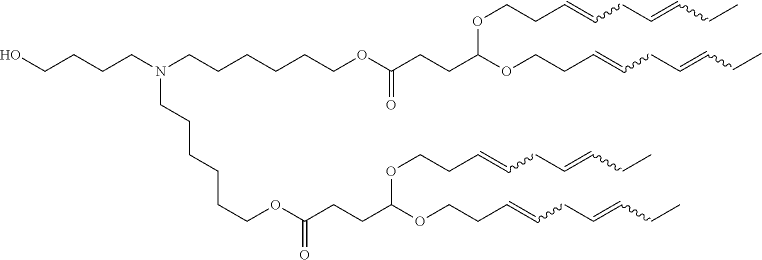 | 84 |
| 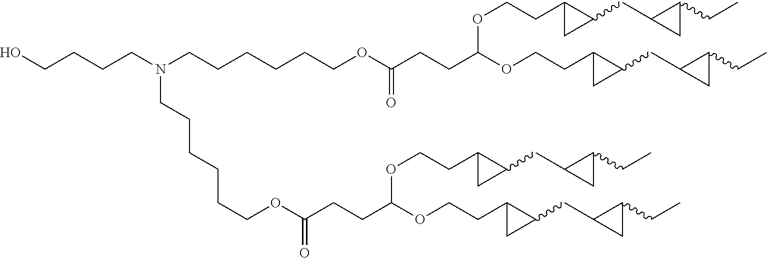 [SMILES: CCC1CC1CC1CC1CCOC(CCC(=O)OCCCCCCN(CCCCO)CCCCCCOC(=O)CCC(OCCC1CC1CC1CC1CC)OCCC1CC1CC1CC1CC)OCCC1CC1CC1CC1CC] | 85 |
| 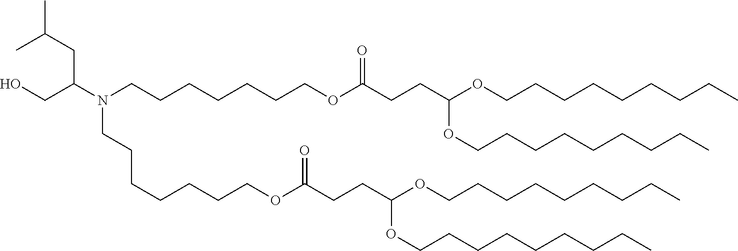 [SMILES: CCCCCCCCCOC(CCC(=O)OCCCCCCCN(CCCCCCCOC(=O)CCC(OCCCCCCCCC)OCCCCCCCCC)C(CO)CC(C)C)OCCCCCCCCC] | 86 |
| 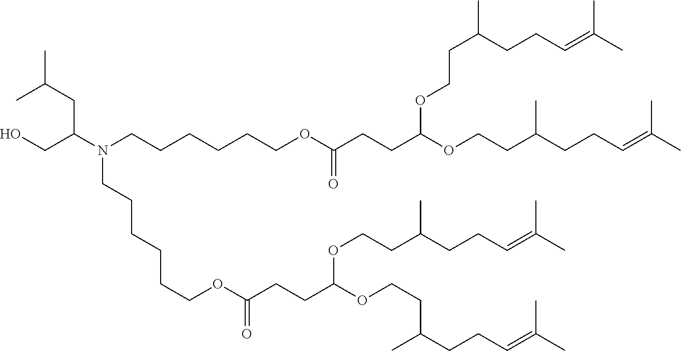 [SMILES: CC(C)=CCCC(C)CCOC(CCC(=O)OCCCCCCN(CCCCCCOC(=O)CCC(OCCC(C)CCC=C(C)C)OCCC(C)CCC=C(C)C)C(CO)CC(C)C)OCCC(C)CCC=C(C)C] | 87 |
| 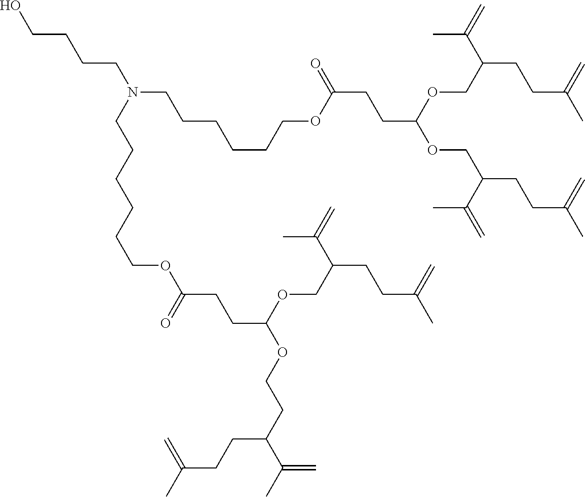 [SMILES: C=C(C)CCC(CCOC(CCC(=O)OCCCCCCN(CCCCO)CCCCCCOC(=O)CCC(OCC(CCC(=C)C)C(=C)C)OCC(CCC(=C)C)C(=C)C)OCC(CCC(=C)C)C(=C)C)C(=C)C] | 88 |
| 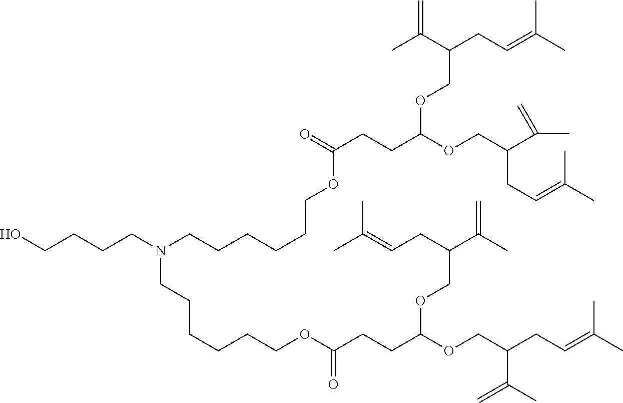 [SMILES: C=C(C)C(CC=C(C)C)COC(CCC(=O)OCCCCCCN(CCCCO)CCCCCCOC(=O)CCC(OCC(CC=C(C)C)C(=C)C)OCC(CC=C(C)C)C(=C)C)OCC(CC=C(C)C)C(=C)C] | 89 |

In some embodiments, an LNP of the present disclosure comprises an ionizable lipid disclosed in PCT Application Publication WO2023044333A1, which is incorporated by reference herein, in its entirety.

### Formula (CY)

In some embodiments, an LNP disclosed herein comprises an ionizable lipid of Formula (CY)

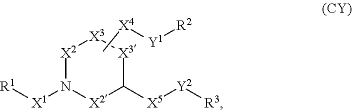

or a pharmaceutically acceptable salt thereof,
wherein:
R is selected from the group consisting of —OH, —OAc, R^{1a},

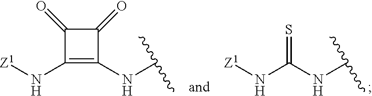

Z¹ is optionally substituted C₁-C₆ alkyl;
X¹ is optionally substituted C₂-C₆ alkylenyl;
X² is selected from the group consisting of a bond, —CH₂— and —CH₂CH₂—;
X^{2′} is selected from the group consisting of a bond, —CH₂— and —CH₂CH₂—;
X³ is selected from the group consisting of a bond, —CH₂— and —CH₂CH₂—;
X^{3′} is selected from the group consisting of a bond, —CH₂— and —CH₂CH₂—;
X⁴ and X⁵ are independently optionally substituted C₂-C₁₄ alkylenyl or optionally substituted C₂-C₁₄ alkenylenyl;
Y¹ and Y² are independently selected from the group consisting of

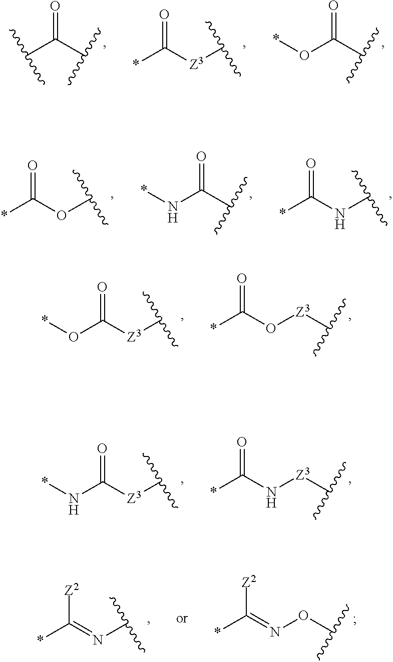

wherein the bond marked with an “*” is attached to X⁴ or X;
each Z² is independently H or optionally substituted C₁-C₈ alkyl;
each Z³ is independently optionally substituted C₁-C₆ alkylenyl;
R² is selected from the group consisting of optionally substituted C₄-C₂₀ alkyl, optionally substituted C₂-C₁₄ alkenyl, and —(CH₂)ₚCH(OR⁶)(OR⁷);
R³ is selected from the group consisting of optionally substituted C₄-C₂₀ alkyl, optionally substituted C₂-C₁₄ alkenyl, or —(CH₂)CH(OR⁸)(OR⁹);
R¹¹ is:

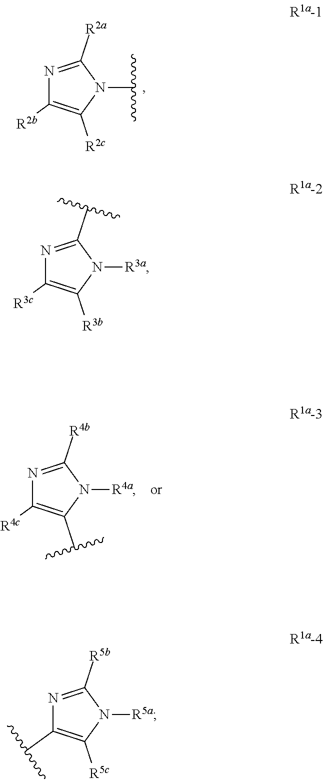

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl;
R^{5a}, R^{5b}, and R^{5c} are independently hydrogen and C₁-C₆ alkyl;
R⁶, R⁷, R⁸, and R⁹ are independently optionally substituted C₁-C₁₄ alkyl, optionally substituted C₂-C₁₄ alkenyl, or —(CH₂)ₘ-A-(CH₂)ₙH;
each A is independently a C₃-C₈ cycloalkylenyl;
each m is independently 0, 1, 2, 3, 4, 5, 6, 7, 8, 9, 10, 11, or 12;
each n is independently 0, 1, 2, 3, 4, 5, 6, 7, 8, 9, 10, 11, or 12;
p is selected from the group consisting of 0, 1, 2, 3, 4, 5, 6, and 7; and
q is selected from the group consisting of 0, 1, 2, 3, 4, 5, 6, and 7.

### Formulas (CY-I), (CY-II), (CY-III), (CY-IV), and (CY-V)

In some embodiments, the present disclosure includes a compound of Formula (CY-I), (CY-II), (CY-III), (CY-IV), or (CY-V):

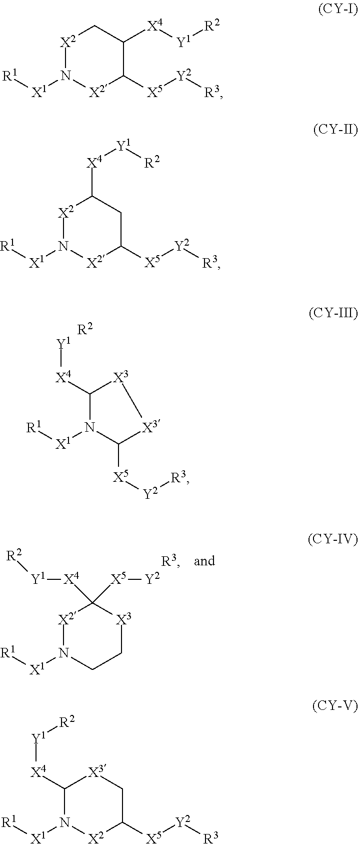

or a pharmaceutically acceptable salt thereof,
wherein X¹, X², X², X³, X^{3′}, X⁴, X⁵, Y¹, Y², R, R², and R³ are defined herein.

### Formulas (CY-VI) and (CY-VII)

In some embodiments, the present disclosure includes a compound of Formula (CY-VI) or (CY-VII):

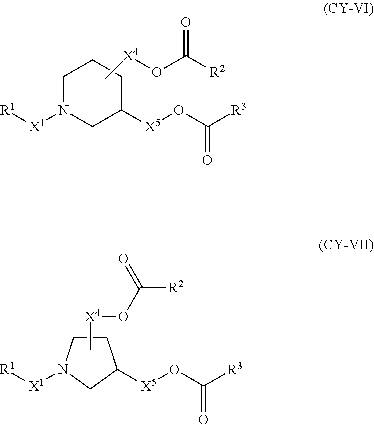

or a pharmaceutically acceptable salt thereof,
wherein X¹, X⁴, X⁵, R¹, R², and R³ are defined herein.

### Formulas (CY-VIII) and (CY-IX)

In some embodiments, the present disclosure includes a compound of Formula (CY-VIII) or (CY-IX):

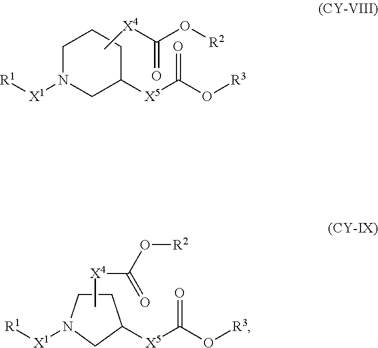

or pharmaceutically acceptable salt thereof
wherein X¹, X⁴, X⁵, R¹, R², and R³ are defined herein.

### Formulas (CY-IV-a), (CY-IV-b), and (CY-IV-c)

In some embodiments, the present disclosure includes a compound of Formula (CY-IV-a), (CY-IV-b), or (CY-IV-c)

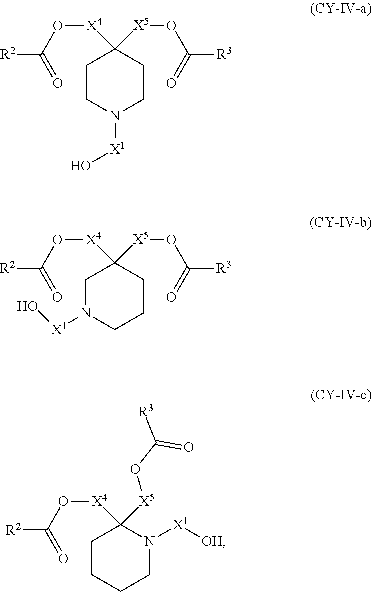

or pharmaceutically acceptable salt thereof.
wherein X¹, X⁴, X⁵, R², and R³ are defined herein. Formulas (CY-IV-d), (CY-IV-e), and (CY-IV-f)

In some embodiments, the present disclosure includes a compound of Formula (CY-IV-d), (CY-IV-e), or (CY-IV-f)

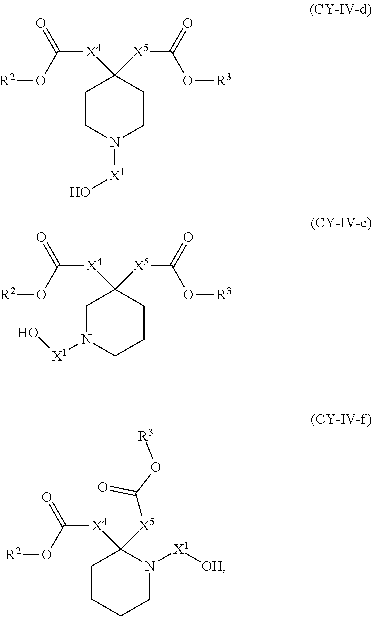

or pharmaceutically acceptable salt thereof.
wherein X¹, X⁴, X⁵, R², and R³ are defined herein.

### Formula CY-IV

In some embodiments, Lipids of the Disclosure have a structure of Formula (CY-IV′):

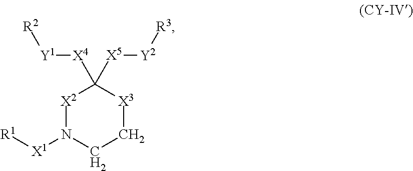

or a pharmaceutically acceptable salt thereof, wherein R¹, R², R³, X¹, X², X³, X⁴, X⁵, Y¹, and Y² are as defined in connection with Formula (CY-I′).

In some embodiments, Lipids of the Disclosure have a structure of Formula (CY-IV′), wherein:

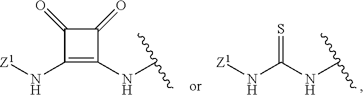

wherein Z¹ is optionally substituted C₁-C₆ alkyl;
X¹ is optionally substituted C₂-C₆ alkylenyl;
X² and X³ are independently a bond, —CH₂—, or —CH₂CH₂—;
X⁴ and X are independently optionally substituted C₂-C₁₄ alkylenyl;
Y¹ and Y² are independently

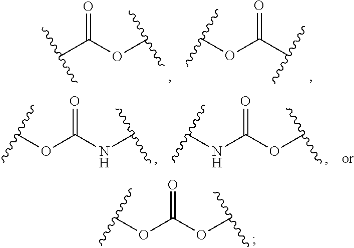 [SMILES: *OC(*)=O ; *OC(*)=O ; *NC(=O)O* ; *NC(=O)O* ; *OC(=O)O*]

R² and R³ are independently optionally substituted C₄-C₂₀ alkyl;
R^{1a} is:

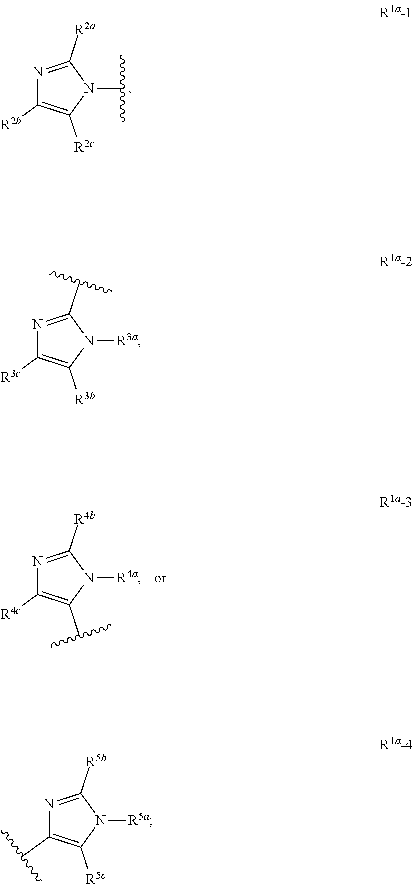

R^{2a}, R^{2b}, and R^{2c} are independently hydrogen and C₁-C₆ alkyl;
R^{3a}, R^{3b}, and R^{3c} are independently hydrogen and C₁-C₆ alkyl;
R^{4a}, R^{4b}, and R^{4c} are independently hydrogen and C₁-C₆ alkyl; and
R^{5a}, R^{5b}, and R^{5c} are independently hydrogen and C₁-C₆ alkyl

In some embodiments, Lipids of the Disclosure have a structure of Formula (CY-IV′), wherein R¹ is —OH,

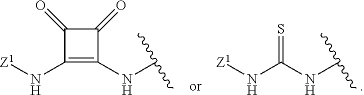

In some embodiments, Lipids of the Disclosure have a structure of Formula (CY-IV′), wherein Y¹ and Y² are independently:

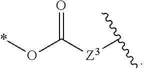

In some embodiments, Lipids of the Disclosure have a structure of Formula (CY-IV′), wherein R² is —CH(OR⁶)(OR⁷).

In some embodiments, Lipids of the Disclosure have a structure of Formula (CY-IV′), wherein R³ is —CH(OR⁸)(OR⁹).

Non-limiting examples of lipids having a structure of Formula (CY-IV′) include compounds CY7, CY8, CY19, CY20, CY21, CY28, CY29, CY40, CY41, CY42, CY48, CY49, CY58, CY59, and CY60.

### Formula (CY-VI′)

In some embodiments, Lipids of the Disclosure have a structure of Formula (CY-VI′):

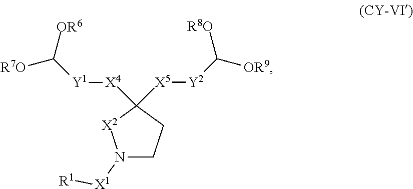

or a pharmaceutically acceptable salt thereof, wherein R¹, R⁶, R⁷, R⁸, R⁹, X¹, X², X³, X⁴, X⁵, Y¹, and Y² are as defined in connection with Formula (CY-I′).

In some embodiments, Lipids of the Disclosure have a structure of Formula (CY-VI′), or a pharmaceutically acceptable salt thereof, wherein R¹ is —OH.

In some embodiments, Lipids of the Disclosure have a structure of Formula (CY-VI′), or a pharmaceutically acceptable salt thereof, wherein X¹ is C₂-C₆ alkylenyl.

In some embodiments, Lipids of the Disclosure have a structure of Formula (CY-VI′), or a pharmaceutically acceptable salt thereof, wherein X² is —CH₂CH₂—.

In some embodiments, Lipids of the Disclosure have a structure of Formula (CY-VI′), or a pharmaceutically acceptable salt thereof, wherein X⁴ is C₂-C₆ alkylenyl.

In some embodiments, Lipids of the Disclosure have a structure of Formula (CY-VI′), or a pharmaceutically acceptable salt thereof, wherein X⁵ is C₂-C₆ alkylenyl.

In some embodiments, Lipids of the Disclosure have a structure of Formula (CY-VI′), or a pharmaceutically acceptable salt thereof, wherein Y¹ is:

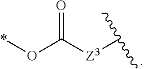

In some embodiments, Lipids of the Disclosure have a structure of Formula (CY-VI′), or a pharmaceutically acceptable salt thereof, wherein Y² is:

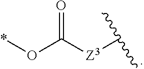

In some embodiments, Lipids of the Disclosure have a structure of Formula (CY-VI′), or a pharmaceutically acceptable salt thereof, wherein each Z³ is independently optionally substituted C₁-C₆ alkylenyl.

In some embodiments, Lipids of the Disclosure have a structure of Formula (CY-VI′), or a pharmaceutically acceptable salt thereof, wherein each Z³ is —CH₂CH₂—.

In some embodiments, Lipids of the Disclosure have a structure of Formula (CY-VI′), or a pharmaceutically acceptable salt thereof, wherein R⁶ is C₅-C₁₄ alkyl.

In some embodiments, Lipids of the Disclosure have a structure of Formula (CY-VI′), or a pharmaceutically acceptable salt thereof, wherein R⁷ is C₅-C₁₄ alkyl.

In some embodiments, Lipids of the Disclosure have a structure of Formula (CY-VI′), or a pharmaceutically acceptable salt thereof, wherein R⁶ is C₆-C₁₄ alkenyl.

In some embodiments, Lipids of the Disclosure have a structure of Formula (CY-VI′), or a pharmaceutically acceptable salt thereof, wherein R⁷ is C₆-C₁₄ alkenyl.

In some embodiments, Lipids of the Disclosure have a structure of Formula (CY-VI′), or a pharmaceutically acceptable salt thereof, wherein R⁸ is C₅-C₁₆ alkyl.

In some embodiments, Lipids of the Disclosure have a structure of Formula (CY-VI′), or a pharmaceutically acceptable salt thereof, wherein R⁹ is C₅-C₁₄ alkyl.

In some embodiments, Lipids of the Disclosure have a structure of Formula (CY-VI′), or a pharmaceutically acceptable salt thereof, wherein R⁸ is C₆-C₁₄ alkenyl.

In some embodiments, Lipids of the Disclosure have a structure of Formula (CY-VI′), or a pharmaceutically acceptable salt thereof, wherein R⁹ is C₆-C₁₄ alkenyl.

In some embodiments, Lipids of the Disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof. In some embodiments, Lipids of the Disclosure are selected from any lipid in Table (II) below or a pharmaceutically acceptable salt thereof:

### R¹

In some embodiments, R′ is selected from the group consisting of —OH, —OAc, R^{1a},

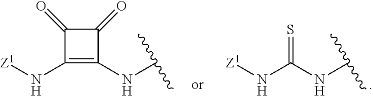

In some embodiments, R¹ is —OH or —OAc. In some embodiments, R¹ is OH. In some embodiments, R¹ is —OAc. In some embodiments, R¹ is R^{1a}. In some embodiments, R¹ is imidazolyl.

In some embodiments, R¹ is

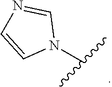 [SMILES: *n1ccnc1]

### R²

In some embodiments, R² is selected from the group consisting of optionally substituted C₄-C₂₀ alkyl, optionally substituted C₂-C₁₄ alkenyl, and —(CH₂)ₚCH(OR⁶)(OR⁷).

In some embodiments, R² is optionally substituted C₄-C₂₀ alkyl. In some embodiments, R² is optionally substituted C₈-C₁₇ alkyl. In some embodiments, R² is optionally substituted C₉-C₁₆ alkyl. In some embodiments, R² is optionally substituted C₈-C₁₀ alkyl. In some embodiments, R² is optionally substituted C₁₁-C₁₃ alkyl. In some embodiments, R² is optionally substituted C14-C₁₆ alkyl. In some embodiments, R² is optionally substituted C₉ alkyl. In some embodiments, R² is optionally substituted C₁₀ alkyl. In some embodiments, R² is optionally substituted C₁₁ alkyl. In some embodiments, R² is optionally substituted C₁₂ alkyl. In some embodiments, R² is optionally substituted C₁₃ alkyl. In some embodiments, R² is optionally substituted C14 alkyl. In some embodiments, R² is optionally substituted C₁₅ alkyl. In some embodiments, R² is optionally substituted C₁₆ alkyl.

In some embodiments, R² is optionally substituted C₂-C₁₄ alkenyl. In some embodiments, R² is optionally substituted C₅-C₁₄ alkenyl. In some embodiments, R² is optionally substituted C₇-C₁₄ alkenyl. In some embodiments, R² is optionally substituted C₉-C₁₄ alkenyl. In some embodiments, R² is optionally substituted C₁₀-C₁₄ alkenyl. In some embodiments, R² is optionally substituted C₁₂-C₁₄ alkenyl.

In some embodiments, R² is —(CH₂)ₚCH(OR⁶)(OR⁷). In some embodiments, R² is —CH(OR⁶)(OR⁷). In some embodiments, R² is —CH₂CH(OR⁶)(OR⁷). In some embodiments, R² is —(CH₂)₂CH(OR⁶)(OR⁷). In some embodiments, R² is —(CH₂)₃CH(OR⁶)(OR⁷). In some embodiments, R² is —(CH₂)₄CH(OR⁶)(OR⁷).

In some embodiments, R² is selected from the group consisting of

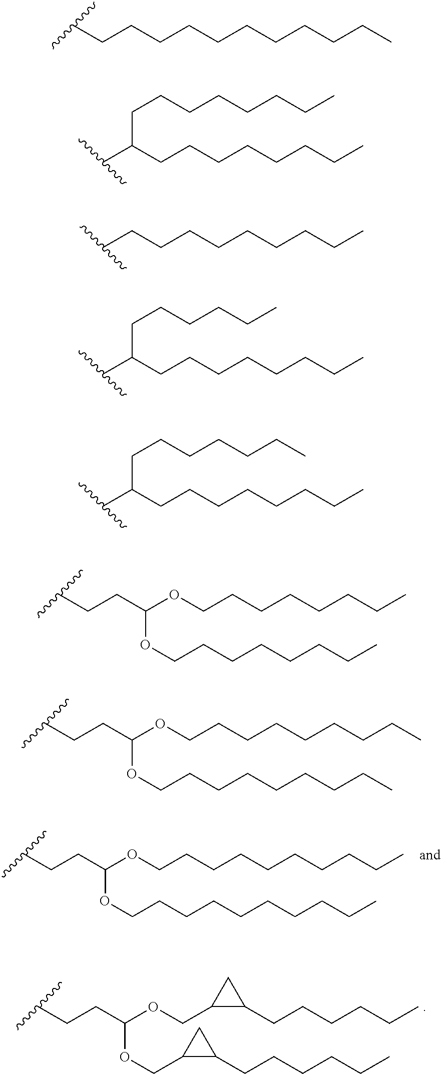

### R³

In some embodiments, R³ is selected from the group consisting of optionally substituted C₄-C₂₀ alkyl, optionally substituted C₂-C₁₄ alkenyl, and —(CH₂)_{q}CH(OR⁶)(OR⁷).

In some embodiments, R³ is optionally substituted C₄-C₂₀ alkyl. In some embodiments, R³ is optionally substituted C₈-C₁₇ alkyl. In some embodiments, R³ is optionally substituted C₉-C₁₆ alkyl. In some embodiments, R³ is optionally substituted C₈-C₁₀ alkyl. In some embodiments, R³ is optionally substituted C₁₁-C₁₃ alkyl. In some embodiments, R³ is optionally substituted C₁₄-C₁₆ alkyl. In some embodiments, R³ is optionally substituted C₉ alkyl. In some embodiments, R³ is optionally substituted C₁₀ alkyl. In some embodiments, R³ is optionally substituted C₁₁ alkyl. In some embodiments, R³ is optionally substituted C12 alkyl. In some embodiments, R³ is optionally substituted C₁₃ alkyl. In some embodiments, R³ is optionally substituted C₁₄ alkyl. In some embodiments, R³ is optionally substituted C₁₅ alkyl. In some embodiments, R³ is optionally substituted C₁₆ alkyl.

In some embodiments, R³ is optionally substituted C₂-C₁₄ alkenyl. In some embodiments, R³ is optionally substituted C₅-C₁₄ alkenyl. In some embodiments, R³ is optionally substituted C₇-C₁₄ alkenyl. In some embodiments, R³ is optionally substituted C₉-C₁₄ alkenyl. In some embodiments, R³ is optionally substituted C₁₀-C₁₄ alkenyl. In some embodiments, R³ is optionally substituted C₁₂-C₁₄ alkenyl.

In some embodiments, R³ is —(CH₂)_{q}CH(OR⁸)(OR⁹). In some embodiments, R³ is —CH(OR⁸)(OR⁹). In some embodiments, R³ is —CH₂CH(OR⁸)(OR⁹). In some embodiments, R³ is —(CH₂)₂CH(OR⁸)(OR⁹). In some embodiments, R³ is —(CH₂)₃CH(OR⁸)(OR⁹). In some embodiments, R³ is —(CH₂)₄CH(OR⁸)(OR⁹).

In some embodiments, R³ is selected from the group consisting of

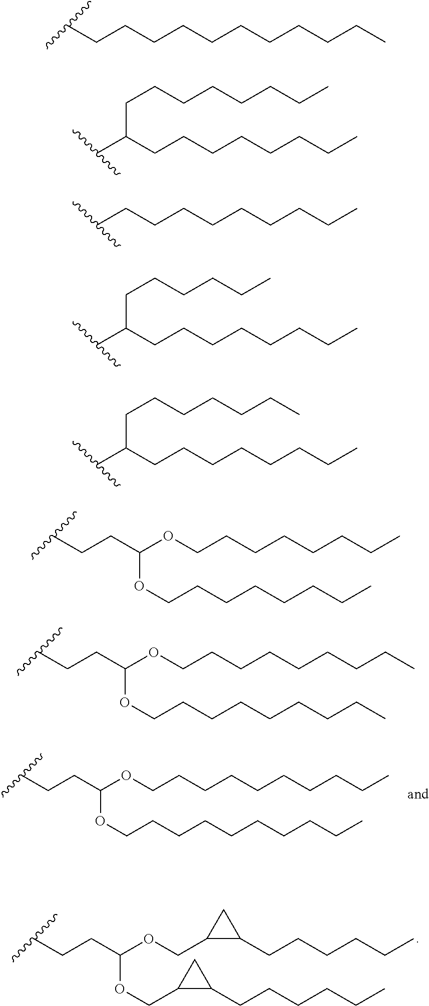

R⁶, R⁷, R⁸, R⁹

In some embodiments, R⁶, R⁷, R⁸, and R⁹ are independently optionally substituted C₁-C₁₄ alkyl, optionally substituted C₂-C₁₄ alkenyl, or —(CH₂)ₘ-A-(CH₂)ₙH. In some embodiments, R⁶, R⁷, R⁸, and R⁹ are independently optionally substituted C₁-C₁₄ alkyl. In some embodiments, R⁶, R⁷, R⁸, and R⁹ are independently optionally substituted C₂-C₁₄ alkenyl. In some embodiments, R⁶, R⁷, R⁸, and R⁹ are independently —(CH₂)ₘ-A-(CH₂)ₙH.

In some embodiments, R⁶ is optionally substituted C₁-C₁₄ alkyl, optionally substituted C₂-C₁₄ alkenyl, or —(CH₂)ₘ-A-(CH₂)ₙH. In some embodiments, R⁶ is optionally substituted C₃-C₁₀ alkyl. In some embodiments, R⁶ is optionally substituted C₄-C₁₀ alkyl. In some embodiments, R⁶ is independently optionally substituted C₅-C₁₀ alkyl. In some embodiments, R⁶ is optionally substituted C₉-C₁₀ alkyl. In some embodiments, R⁶ is optionally substituted C₁-C₁₄ alkyl. In some embodiments, R⁶ is optionally substituted C₂-C₁₄ alkenyl. In some embodiments, R⁶ is —(CH₂)ₘ-A-(CH₂)ₙH.

In some embodiments, R⁷ is optionally substituted C₁-C₁₄ alkyl, optionally substituted C₂-C₁₄ alkenyl, or —(CH₂)ₘ-A-(CH₂)ₙH. In some embodiments, R⁷ is optionally substituted C₃-C₁₀ alkyl. In some embodiments, R⁷ is optionally substituted C₄-C₁₀ alkyl. In some embodiments, R⁷ is optionally substituted C₅-C₁₀ alkyl. In some embodiments, R⁷ is optionally substituted C₉-C₁₀ alkyl. In some embodiments, R⁷ is optionally substituted C₁-C₁₄ alkyl. In some embodiments, R⁷ is optionally substituted optionally substituted C₂-C₁₄ alkenyl. In some embodiments, R⁷ is —(CH₂)ₘ-A-(CH₂)ₙH.

In some embodiments, R⁸ is optionally substituted C₁-C₁₄ alkyl, optionally substituted C₂-C₁₄ alkenyl, or (CH₂)ₘ-A-(CH₂)ₙH. In some embodiments, R⁸ is optionally substituted C₃-C₁₀ alkyl. In some embodiments, R⁸ is optionally substituted C₄-C₁₀ alkyl. In some embodiments, R⁸ is optionally substituted C₅-C₁₀ alkyl. In some embodiments, R⁸ is optionally substituted C₉-C₁₀ alkyl. In some embodiments, R⁸ is optionally substituted C₁-C₁₄ alkyl. In some embodiments, R′ is optionally substituted C₂-C₁₄ alkenyl. In some embodiments, R⁸ is —(CH₂)ₘ-A-(CH₂)ₙH.

In some embodiments, R⁹ is optionally substituted C₁-C₁₄ alkyl, optionally substituted C₂-C₁₄ alkenyl, or —(CH₂)ₘ-A-(CH₂)ₙH. In some embodiments, R⁹ is optionally substituted C₃-C₁₀ alkyl. In some embodiments, R⁹ is optionally substituted C₄-C₁₀ alkyl. In some embodiments, R⁹ is optionally substituted C₅-C₁₀ alkyl. In some embodiments, R⁹ is optionally substituted C₉-C₁₀ alkyl. In some embodiments, R⁹ is optionally substituted C₁-C₁₄ alkyl. In some embodiments, R⁹ is optionally substituted C₂-C₁₄ alkenyl. In some embodiments, R⁹ is —(CH₂)ₘ-A-(CH₂)ₙH.

In some embodiments, each m is independently 0, 1, 2, 3, 4, 5, 6, 7, 8, 9, 10, 11, or 12. In some embodiments, each m is 0. In some embodiments, each m is 1. In some embodiments, each m is 2. In some embodiments, each m is 3. In some embodiments, each m is 4. In some embodiments, each m is 5. In some embodiments, each m is 6. In some embodiments, each m is 7. In some embodiments, each m is 8. In some embodiments, each m is 9. In some embodiments, each m is 10. In some embodiments, each m is 11. In some embodiments, each m is 12.

In some embodiments, each n is independently 0, 1, 2, 3, 4, 5, 6, 7, 8, 9, 10, 11, or 12. In some embodiments, each n is 0. In some embodiments, each n is 1. In some embodiments, each n is 2. In some embodiments, each n is 3. In some embodiments, each n is 4. In some embodiments, each n is 5. In some embodiments, each n is 6. In some embodiments, each n is 7. In some embodiments, each n is 8. In some embodiments, each n is 9. In some embodiments, each n is 10. In some embodiments, each n is 11. In some embodiments, each n is 12.

In some embodiments, each A is independently a C₃-C₈ cycloalkylenyl. In some embodiments, each A is cyclopropylenyl.

### X¹

In some embodiments, X1 is optionally substituted C2-C6 alkylenyl. In some embodiments, X1 is optionally substituted C2-C5 alkylenyl. In some embodiments, X1 is optionally substituted C2-C4 alkylenyl. In some embodiments, X1 is optionally substituted C2-C3 alkylenyl. In some embodiments, X1 is optionally substituted C2 alkylenyl. In some embodiments, X1 is optionally substituted C3 alkylenyl. In some embodiments, X1 is optionally substituted C4 alkylenyl. In some embodiments, X1 is optionally substituted C5 alkylenyl. In some embodiments, X1 is optionally substituted C6 alkylenyl. In some embodiments, X1 is optionally substituted —(CH2)2-. In some embodiments, X1 is optionally substituted —(CH2)3-. In some embodiments, XI is optionally substituted —(CH2)4-. In some embodiments, X1 is optionally substituted —(CH2)5-. In some embodiments, X1 is optionally substituted —(CH₂)₆—.

### X²

In some embodiments, X2 is selected from the group consisting of a bond, —CH2- and —CH2CH2-. In some embodiments, X2 is a bond. In some embodiments, X2 is —CH2-. In some embodiments, X2 is —CH2CH2-.

### X^{2′}

In some embodiments, X^{2′} is selected from the group consisting of a bond, —CH₂— and —CH₂CH₂—. In some embodiments, X^{2′} is a bond. In some embodiments, X^{2′} is —CH₂—. In some embodiments, X^{2′} is —CH₂CH₂—.

### X³

In some embodiments, X³ is selected from the group consisting of a bond, —CH₂— and —CH₂CH₂—. In some embodiments, X³ is a bond. In some embodiments, X³ is —CH₂—. In some embodiments, X³ is —CH₂CH₂—.

### X^{3′}

In some embodiments, X^{3′} is selected from the group consisting of a bond, —CH₂— and —CH₂CH₂—. In some embodiments, X^{3′} is a bond. In some embodiments, X^{3′} is —CH₂—. In some embodiments, X^{3′} is —CH₂CH₂—.

### X⁴

In some embodiments, X⁴ is selected from the group consisting of optionally substituted C₂-C₁₄ alkylenyl and optionally substituted C₂-C₁₄ alkenylenyl. In some embodiments, X⁴ is optionally substituted C₂-C₁₄ alkylenyl. In some embodiments, X⁴ is optionally substituted C₂-C₁₀ alkylenyl. In some embodiments, X⁴ is optionally substituted C₂-C₈ alkylenyl. In some embodiments, X⁴ is optionally substituted C₂-C₆ alkylenyl. In some embodiments, X⁴ is optionally substituted C₃-C₆ alkylenyl. In some embodiments, X⁴ is optionally substituted C₃ alkylenyl. In some embodiments, X⁴ is optionally substituted C₄ alkylenyl. In some embodiments, X⁴ is optionally substituted C₅ alkylenyl. In some embodiments, X⁴ is optionally substituted C₆ alkylenyl. In some embodiments, X⁴ is optionally substituted —(CH₂)₂—. In some embodiments, X⁴ is optionally substituted —(CH₂)₃—. In some embodiments, X⁴ is optionally substituted —(CH₂)₄—. In some embodiments, X⁴ is optionally substituted —(CH₂)₅—. In some embodiments, X⁴ is optionally substituted —(CH₂)₆—.

### X⁵

In some embodiments, X⁵ is selected from the group consisting of optionally substituted C₂-C₁₄ alkylenyl and optionally substituted C₂-C₁₄ alkenylenyl. In some embodiments, X⁵ is optionally substituted C₂-C₁₄ alkylenyl. In some embodiments, X⁵ is optionally substituted C₂-C₁₀ alkylenyl. In some embodiments, X⁵ is optionally substituted C₂-C₈ alkylenyl. In some embodiments, X⁵ is optionally substituted C₂-C₆ alkylenyl. In some embodiments, X⁵ is optionally substituted C₃-C₆ alkylenyl. In some embodiments, X⁵ is optionally substituted C₃ alkylenyl. In some embodiments, X⁵ is optionally substituted C₄ alkylenyl. In some embodiments, X⁵ is optionally substituted C₅ alkylenyl. In some embodiments, X⁵ is optionally substituted C₆ alkylenyl. In some embodiments, X⁵ is optionally substituted —(CH₂)₂—. In some embodiments, X⁵ is optionally substituted —(CH₂)₃—. In some embodiments, X⁵ is optionally substituted —(CH₂)₄—. In some embodiments, X⁵ is optionally substituted —(CH₂)₅—. In some embodiments, X⁵ is optionally substituted —(CH₂)₆—.

### Y¹

In some embodiments, Y¹ is selected from the group consisting of

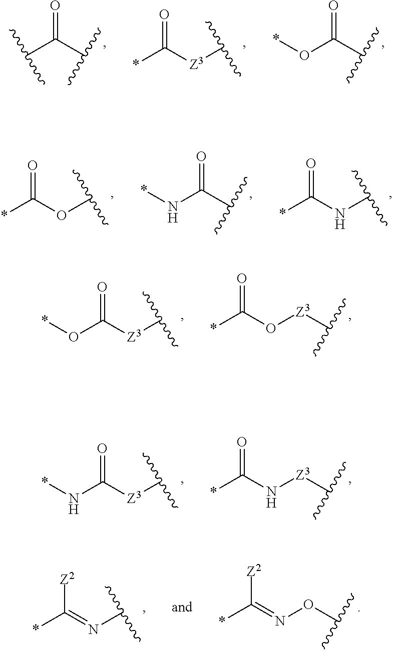

In some embodiments, Y¹ is

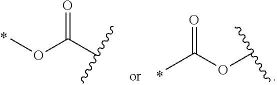

### Y²

In some embodiments, Y² is selected from the group consisting of

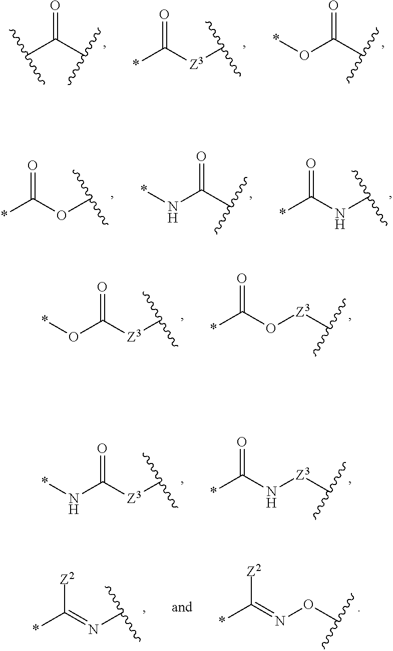

In some embodiments, Y² is

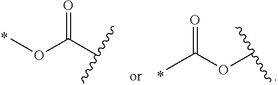

TABLE (II)
Non-Limiting Examples of Ionizable Lipids with a Cyclic Core

| | Compound |
|---|---|
| Structure | No. |
| 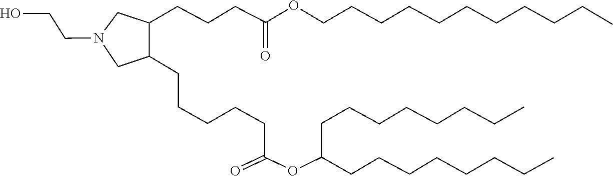 [SMILES: CCCCCCCCCCCOC(=O)CCCC1CN(CCO)CC1CCCCCC(=O)OC(CCCCCCCC)CCCCCCCC] | CY1 |
| 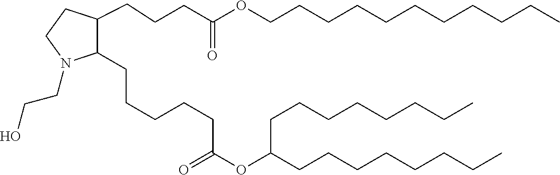 [SMILES: CCCCCCCCCCCOC(=O)CCCC1CCN(CCO)C1CCCCCC(=O)OC(CCCCCCCC)CCCCCCCC] | CY2 |
| 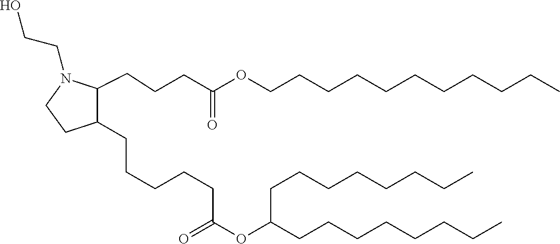 [SMILES: CCCCCCCCCCCOC(=O)CCCC1C(CCCCCC(=O)OC(CCCCCCCC)CCCCCCCC)CCN1CCO] | CY3 |
| 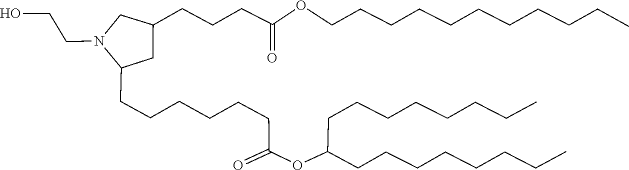 [SMILES: CCCCCCCCCCCOC(=O)CCCC1CC(CCCCCCC(=O)OC(CCCCCCCC)CCCCCCCC)N(CCO)C1] | CY4 |
| 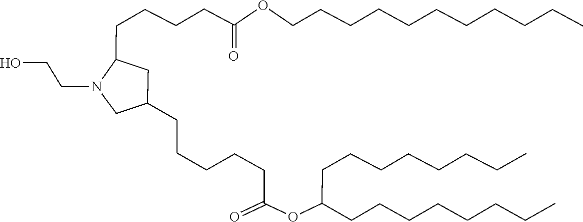 [SMILES: CCCCCCCCCCCOC(=O)CCCCC1CC(CCCCCC(=O)OC(CCCCCCCC)CCCCCCCC)CN1CCO] | CY5 |
| 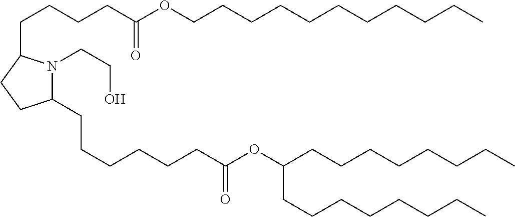 [SMILES: CCCCCCCCCCCOC(=O)CCCCC1CCC(CCCCCCC(=O)OC(CCCCCCCC)CCCCCCCC)N1CCO] | CY6 |
| 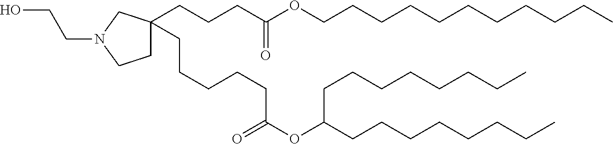 [SMILES: CCCCCCCCCCCOC(=O)CCCC1(CCCCCC(=O)OC(CCCCCCCC)CCCCCCCC)CCN(CCO)C1] | CY7 |
| 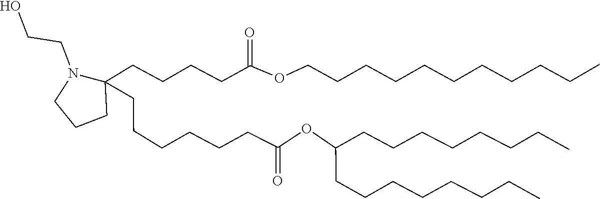 [SMILES: CCCCCCCCCCCOC(=O)CCCCC1(CCCCCCC(=O)OC(CCCCCCCC)CCCCCCCC)CCCN1CCO] | CY8 |
| 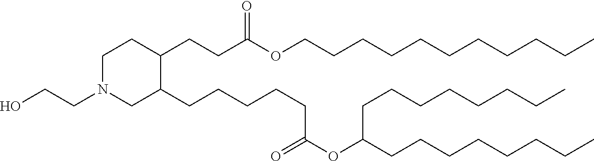 [SMILES: CCCCCCCCCCCOC(=O)CCC1CCN(CCO)CC1CCCCCC(=O)OC(CCCCCCCC)CCCCCCCC] | CY9 |
| 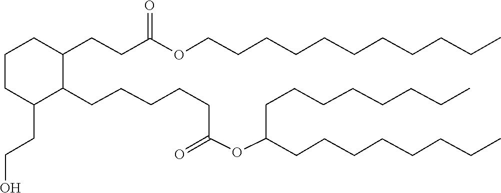 [SMILES: CCCCCCCCCCCOC(=O)CCC1CCCC(CCO)C1CCCCCC(=O)OC(CCCCCCCC)CCCCCCCC] | CY10 |
| 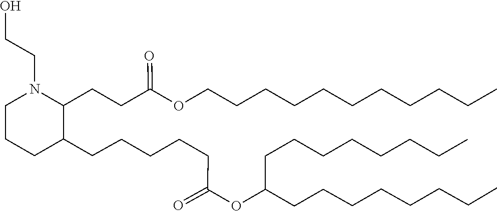 [SMILES: CCCCCCCCCCCOC(=O)CCC1C(CCCCCC(=O)OC(CCCCCCCC)CCCCCCCC)CCCN1CCO] | CY11 |
| 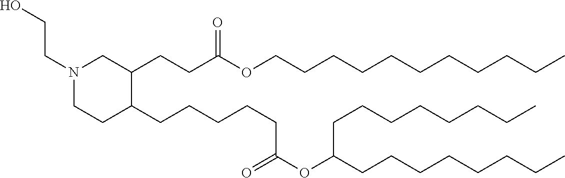 [SMILES: CCCCCCCCCCCOC(=O)CCC1CN(CCO)CCC1CCCCCC(=O)OC(CCCCCCCC)CCCCCCCC] | CY12 |
| 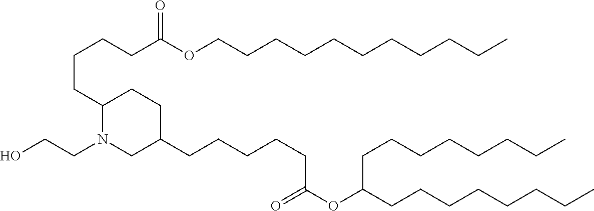 [SMILES: CCCCCCCCCCCOC(=O)CCCCC1CCC(CCCCCC(=O)OC(CCCCCCCC)CCCCCCCC)CN1CCO] | CY13 |
| 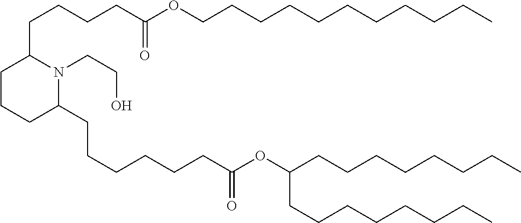 [SMILES: CCCCCCCCCCCOC(=O)CCCCC1CCCC(CCCCCCC(=O)OC(CCCCCCCC)CCCCCCCC)N1CCO] | CY14 |
| 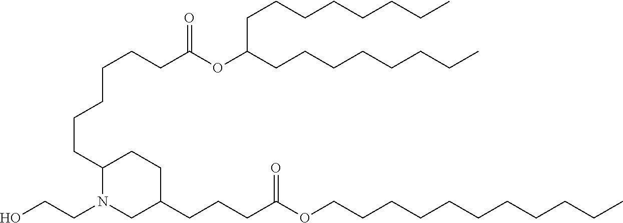 [SMILES: CCCCCCCCCCCOC(=O)CCCC1CCC(CCCCCCC(=O)OC(CCCCCCCC)CCCCCCCC)N(CCO)C1] | CY15 |
| 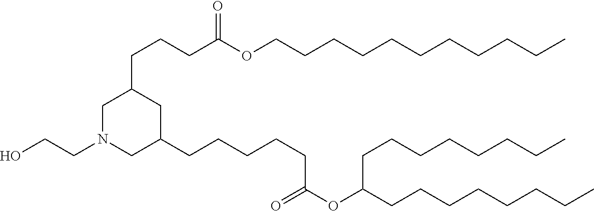 [SMILES: CCCCCCCCCCCOC(=O)CCCC1CC(CCCCCC(=O)OC(CCCCCCCC)CCCCCCCC)CN(CCO)C1] | CY16 |
| 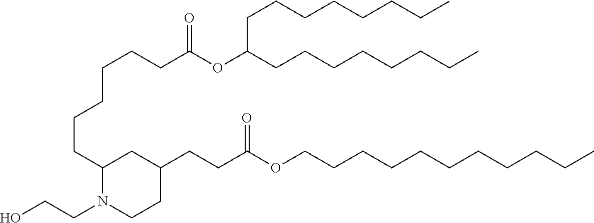 [SMILES: CCCCCCCCCCCOC(=O)CCC1CCN(CCO)C(CCCCCCC(=O)OC(CCCCCCCC)CCCCCCCC)C1] | CY17 |
| 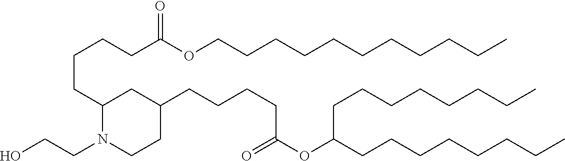 [SMILES: CCCCCCCCCCCOC(=O)CCCCC1CC(CCCCC(=O)OC(CCCCCCCC)CCCCCCCC)CCN1CCO] | CY18 |
| 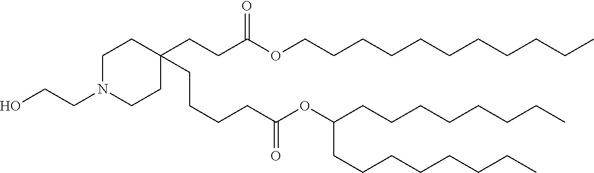 [SMILES: CCCCCCCCCCCOC(=O)CCC1(CCCCC(=O)OC(CCCCCCCC)CCCCCCCC)CCN(CCO)CC1] | CY19 |
| 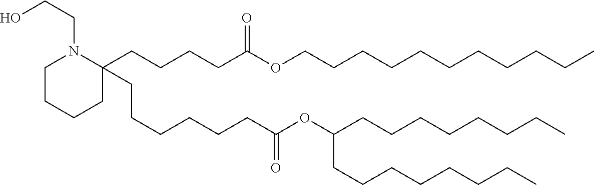 [SMILES: CCCCCCCCCCCOC(=O)CCCCC1(CCCCCCC(=O)OC(CCCCCCCC)CCCCCCCC)CCCCN1CCO] | CY20 |
| 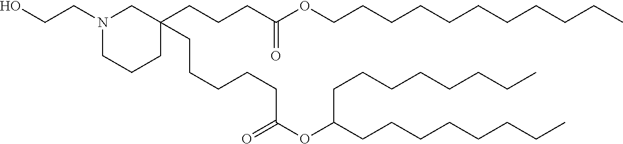 [SMILES: CCCCCCCCCCCOC(=O)CCCC1(CCCCCC(=O)OC(CCCCCCCC)CCCCCCCC)CCCN(CCO)C1] | CY21 |
| 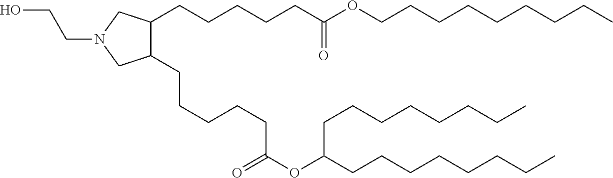 [SMILES: CCCCCCCCCOC(=O)CCCCCC1CN(CCO)CC1CCCCCC(=O)OC(CCCCCCCC)CCCCCCCC] | CY22 |
| 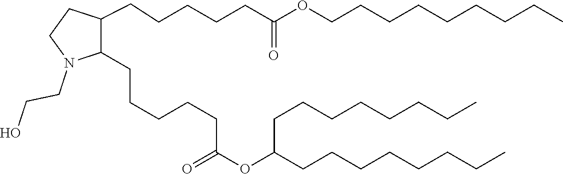 [SMILES: CCCCCCCCCOC(=O)CCCCCC1CCN(CCO)C1CCCCCC(=O)OC(CCCCCCCC)CCCCCCCC] | CY23 |
| 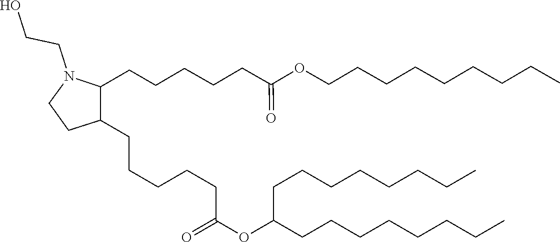 [SMILES: CCCCCCCCCOC(=O)CCCCCC1C(CCCCCC(=O)OC(CCCCCCCC)CCCCCCCC)CCN1CCO] | CY24 |
| 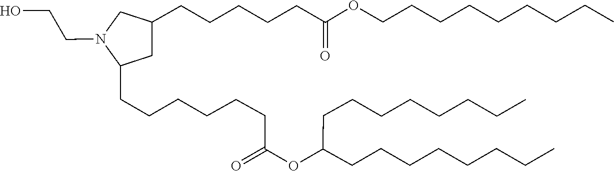 [SMILES: CCCCCCCCCOC(=O)CCCCCC1CC(CCCCCCC(=O)OC(CCCCCCCC)CCCCCCCC)N(CCO)C1] | CY25 |
| 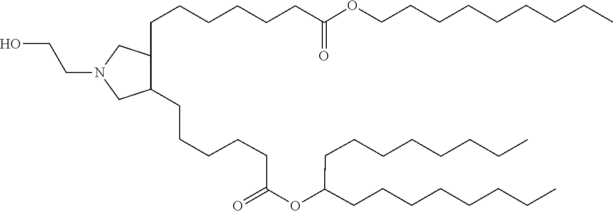 [SMILES: CCCCCCCCCOC(=O)CCCCCCC1CN(CCO)CC1CCCCCC(=O)OC(CCCCCCCC)CCCCCCCC] | CY26 |
| 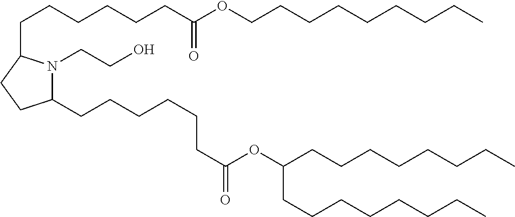 [SMILES: CCCCCCCCCOC(=O)CCCCCCC1CCC(CCCCCCC(=O)OC(CCCCCCCC)CCCCCCCC)N1CCO] | CY27 |
| 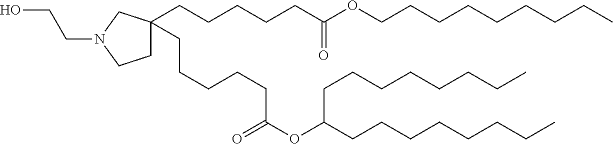 [SMILES: CCCCCCCCCOC(=O)CCCCCC1(CCCCCC(=O)OC(CCCCCCCC)CCCCCCCC)CCN(CCO)C1] | CY28 |
| 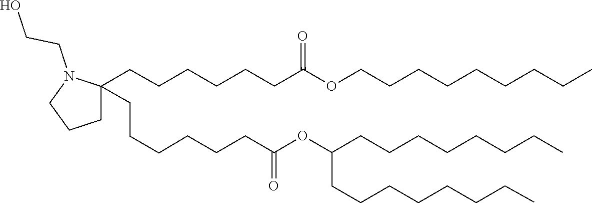 [SMILES: CCCCCCCCCOC(=O)CCCCCCC1(CCCCCCC(=O)OC(CCCCCCCC)CCCCCCCC)CCCN1CCO] | CY29 |
| 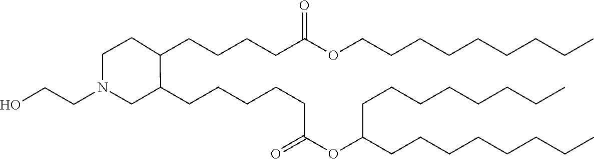 [SMILES: CCCCCCCCCOC(=O)CCCCC1CCN(CCO)CC1CCCCCC(=O)OC(CCCCCCCC)CCCCCCCC] | CY30 |
| 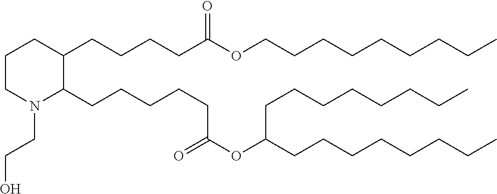 [SMILES: CCCCCCCCCOC(=O)CCCCC1CCCN(CCO)C1CCCCCC(=O)OC(CCCCCCCC)CCCCCCCC] | CY31 |
| 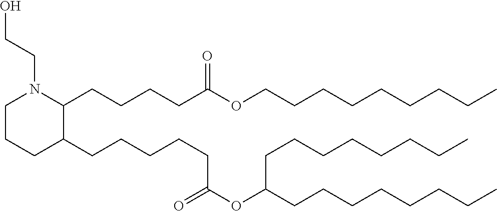 [SMILES: CCCCCCCCCOC(=O)CCCCC1C(CCCCCC(=O)OC(CCCCCCCC)CCCCCCCC)CCCN1CCO] | CY32 |
| 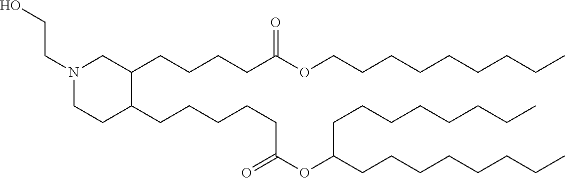 [SMILES: CCCCCCCCCOC(=O)CCCCC1CN(CCO)CCC1CCCCCC(=O)OC(CCCCCCCC)CCCCCCCC] | CY33 |
| 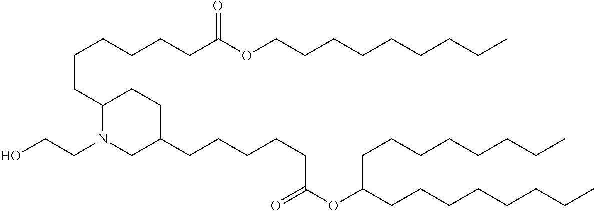 [SMILES: CCCCCCCCCOC(=O)CCCCCCC1CCC(CCCCCC(=O)OC(CCCCCCCC)CCCCCCCC)CN1CCO] | CY34 |
| 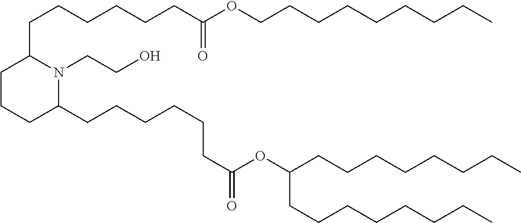 [SMILES: CCCCCCCCCOC(=O)CCCCCCC1CCCC(CCCCCCC(=O)OC(CCCCCCCC)CCCCCCCC)N1CCO] | CY35 |
| 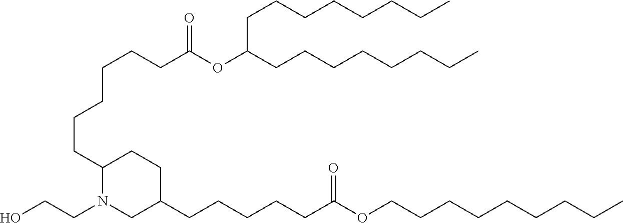 [SMILES: CCCCCCCCCOC(=O)CCCCCC1CCC(CCCCCCC(=O)OC(CCCCCCCC)CCCCCCCC)N(CCO)C1] | CY36 |
| 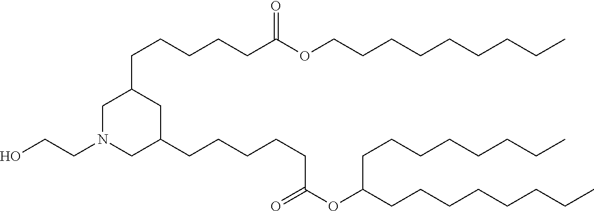 [SMILES: CCCCCCCCCOC(=O)CCCCCC1CC(CCCCCC(=O)OC(CCCCCCCC)CCCCCCCC)CN(CCO)C1] | CY37 |
| 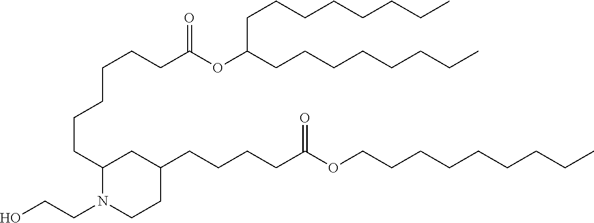 [SMILES: CCCCCCCCCOC(=O)CCCCC1CCN(CCO)C(CCCCCCC(=O)OC(CCCCCCCC)CCCCCCCC)C1] | CY38 |
| 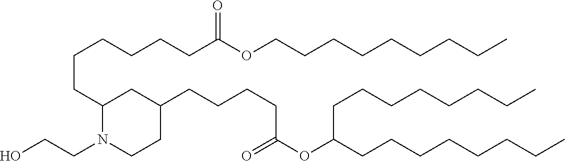 [SMILES: CCCCCCCCCOC(=O)CCCCCCC1CC(CCCCC(=O)OC(CCCCCCCC)CCCCCCCC)CCN1CCO] | CY39 |
| 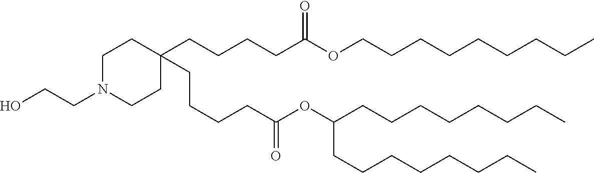 [SMILES: CCCCCCCCCOC(=O)CCCCC1(CCCCC(=O)OC(CCCCCCCC)CCCCCCCC)CCN(CCO)CC1] | CY40 |
| 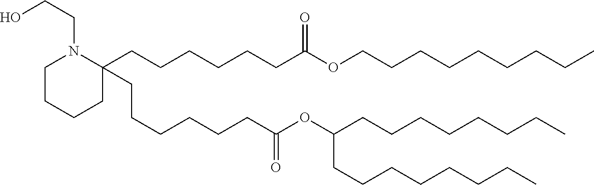 [SMILES: CCCCCCCCCOC(=O)CCCCCCC1(CCCCCCC(=O)OC(CCCCCCCC)CCCCCCCC)CCCCN1CCO] | CY41 |
| 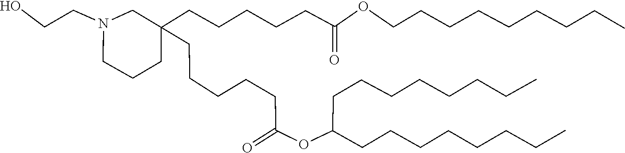 [SMILES: CCCCCCCCCOC(=O)CCCCCC1(CCCCCC(=O)OC(CCCCCCCC)CCCCCCCC)CCCN(CCO)C1] | CY42 |
| 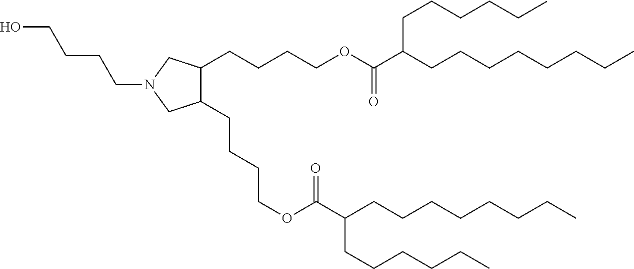 [SMILES: CCCCCCCCC(CCCCCC)C(=O)OCCCCC1CN(CCCCO)CC1CCCCOC(=O)C(CCCCCC)CCCCCCCC] | CY43 |
| 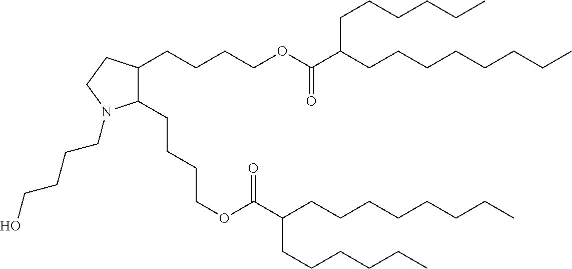 [SMILES: CCCCCCCCC(CCCCCC)C(=O)OCCCCC1CCN(CCCCO)C1CCCCOC(=O)C(CCCCCC)CCCCCCCC] | CY44 |
| 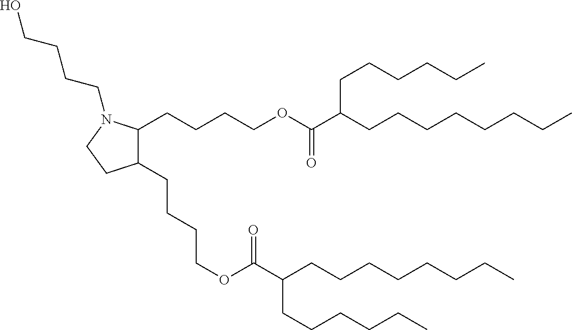 [SMILES: CCCCCCCCC(CCCCCC)C(=O)OCCCCC1CCN(CCCCO)C1CCCCOC(=O)C(CCCCCC)CCCCCCCC] | CY45 |
| 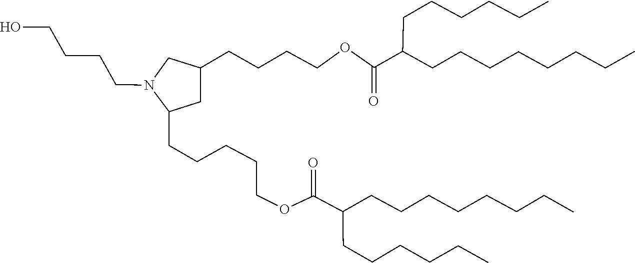 [SMILES: CCCCCCCCC(CCCCCC)C(=O)OCCCCCC1CC(CCCCOC(=O)C(CCCCCC)CCCCCCCC)CN1CCCCO] | CY46 |
| 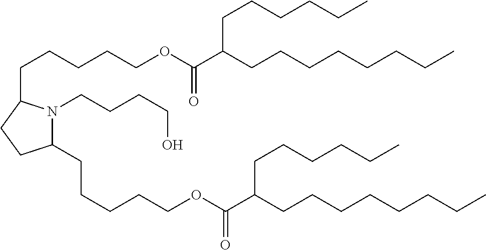 [SMILES: CCCCCCCCC(CCCCCC)C(=O)OCCCCCC1CCC(CCCCCOC(=O)C(CCCCCC)CCCCCCCC)N1CCCCO] | CY47 |
| 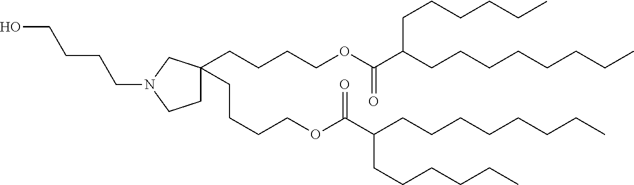 [SMILES: CCCCCCCCC(CCCCCC)C(=O)OCCCCC1(CCCCOC(=O)C(CCCCCC)CCCCCCCC)CCN(CCCCO)C1] | CY48 |
| 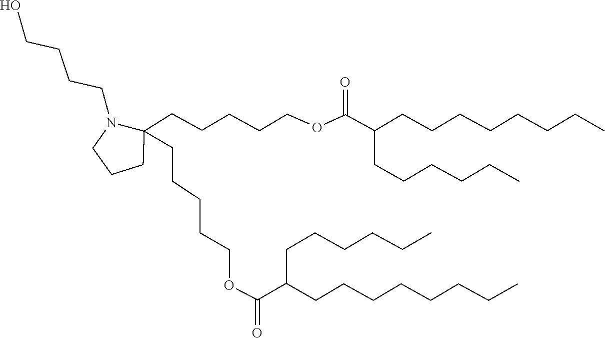 [SMILES: CCCCCCCCC(CCCCCC)C(=O)OCCCCCC1(CCCCCOC(=O)C(CCCCCC)CCCCCCCC)CCCN1CCCCO] | CY49 |
| 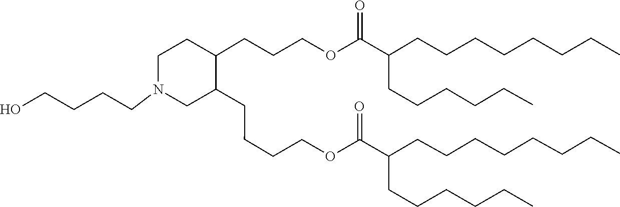 [SMILES: CCCCCCCCC(CCCCCC)C(=O)OCCCCC1CN(CCCCO)CCC1CCCOC(=O)C(CCCCCC)CCCCCCCC] | CY50 |
| 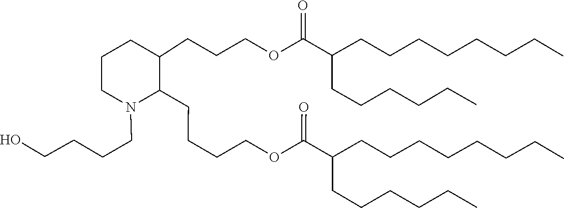 [SMILES: CCCCCCCCC(CCCCCC)C(=O)OCCCCC1C(CCCOC(=O)C(CCCCCC)CCCCCCCC)CCCN1CCCCO] | CY51 |
| 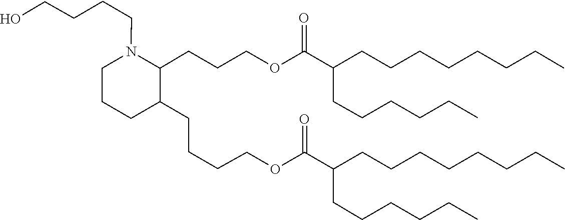 [SMILES: CCCCCCCCC(CCCCCC)C(=O)OCCCCC1CCCN(CCCCO)C1CCCOC(=O)C(CCCCCC)CCCCCCCC] | CY52 |
| 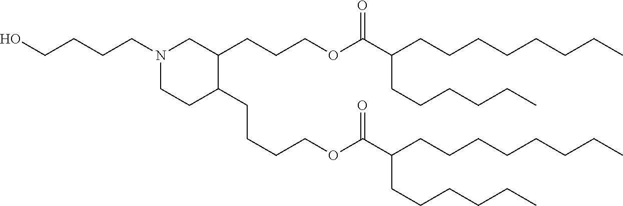 [SMILES: CCCCCCCCC(CCCCCC)C(=O)OCCCCC1CCN(CCCCO)CC1CCCOC(=O)C(CCCCCC)CCCCCCCC] | CY53 |
| 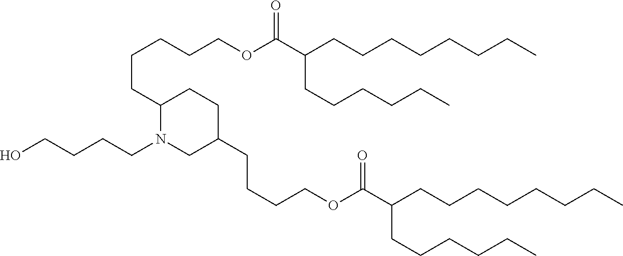 [SMILES: CCCCCCCCC(CCCCCC)C(=O)OCCCCCC1CCC(CCCCOC(=O)C(CCCCCC)CCCCCCCC)CN1CCCCO] | CY54 |
| 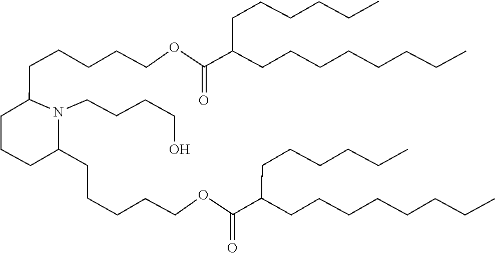 [SMILES: CCCCCCCCC(CCCCCC)C(=O)OCCCCCC1CCCC(CCCCCOC(=O)C(CCCCCC)CCCCCCCC)N1CCCCO] | CY55 |
| 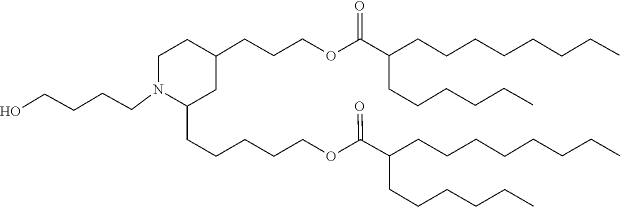 [SMILES: CCCCCCCCC(CCCCCC)C(=O)OCCCCCC1CC(CCCOC(=O)C(CCCCCC)CCCCCCCC)CCN1CCCCO] | CY56 |
| 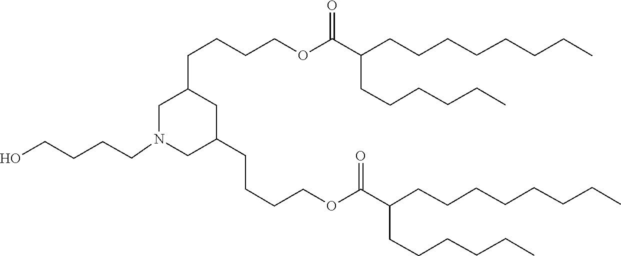 [SMILES: CCCCCCCCC(CCCCCC)C(=O)OCCCCC1CC(CCCCOC(=O)C(CCCCCC)CCCCCCCC)CN(CCCCO)C1] | CY57 |
| 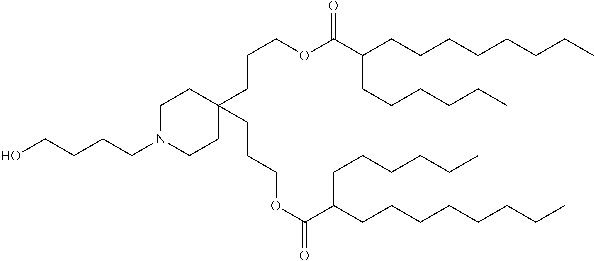 [SMILES: CCCCCCCCC(CCCCCC)C(=O)OCCCC1(CCCOC(=O)C(CCCCCC)CCCCCCCC)CCN(CCCCO)CC1] | CY58 |
| 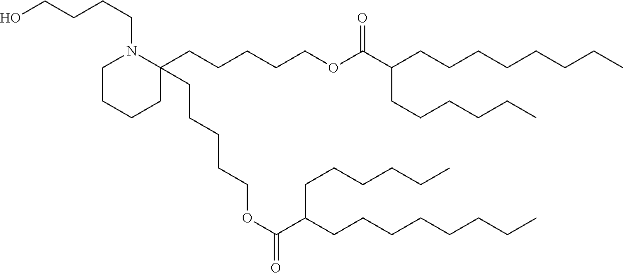 [SMILES: CCCCCCCCC(CCCCCC)C(=O)OCCCCCC1(CCCCCOC(=O)C(CCCCCC)CCCCCCCC)CCCCN1CCCCO] | CY59 |
| 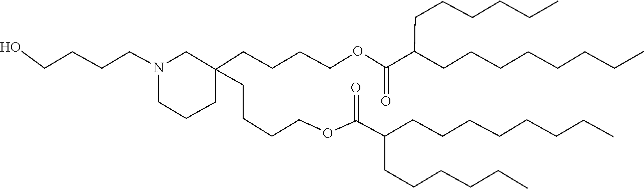 [SMILES: CCCCCCCCC(CCCCCC)C(=O)OCCCCC1(CCCCOC(=O)C(CCCCCC)CCCCCCCC)CCCN(CCCCO)C1] | CY60 |
| 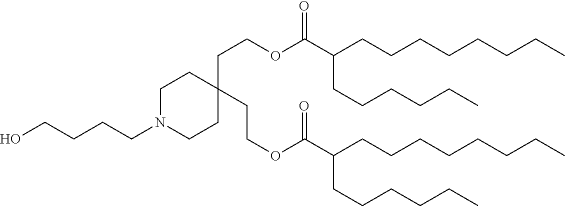 [SMILES: CCCCCCCCC(CCCCCC)C(=O)OCCC1(CCOC(=O)C(CCCCCC)CCCCCCCC)CCN(CCCCO)CC1] | CY61 |
| 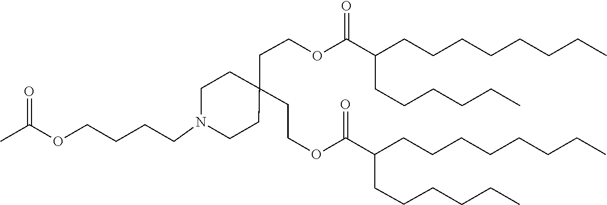 [SMILES: CCCCCCCCC(CCCCCC)C(=O)OCCC1(CCOC(=O)C(CCCCCC)CCCCCCCC)CCN(CCCCOC(C)=O)CC1] | CY62 |
| 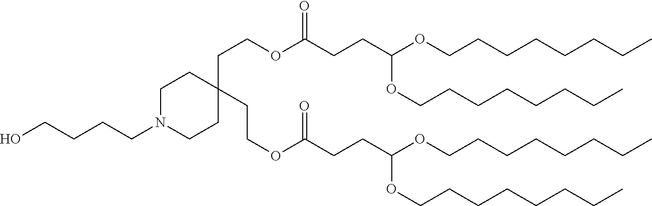 [SMILES: CCCCCCCCOC(CCC(=O)OCCC1(CCOC(=O)CCC(OCCCCCCCC)OCCCCCCCC)CCN(CCCCO)CC1)OCCCCCCCC] | CY63 |
| 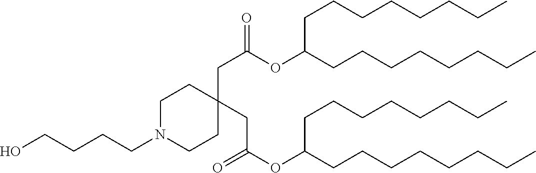 [SMILES: CCCCCCCCC(CCCCCCCC)OC(=O)CC1(CC(=O)OC(CCCCCCCC)CCCCCCCC)CCN(CCCCO)CC1] | CY64 |
| 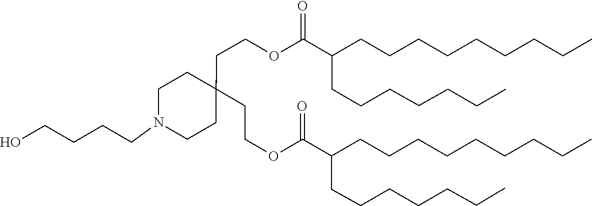 [SMILES: CCCCCCCCCC(CCCCCCC)C(=O)OCCC1(CCOC(=O)C(CCCCCCC)CCCCCCCCC)CCN(CCCCO)CC1] | CY65 |
| 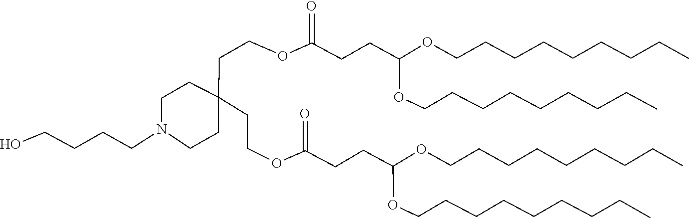 [SMILES: CCCCCCCCCOC(CCC(=O)OCCC1(CCOC(=O)CCC(OCCCCCCCCC)OCCCCCCCCC)CCN(CCCCO)CC1)OCCCCCCCCC] | CY66 |
| 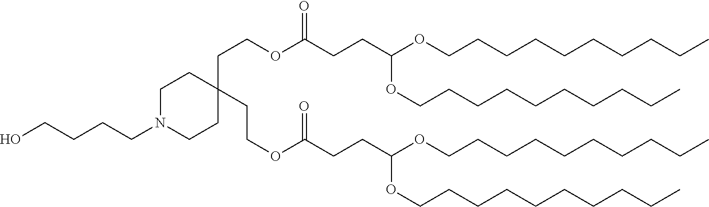 [SMILES: CCCCCCCCCCOC(CCC(=O)OCCC1(CCOC(=O)CCC(OCCCCCCCCCC)OCCCCCCCCCC)CCN(CCCCO)CC1)OCCCCCCCCCC] | CY67 |
| 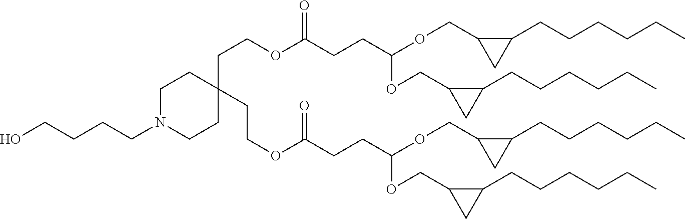 [SMILES: CCCCCCC1CC1COC(CCC(=O)OCCC1(CCOC(=O)CCC(OCC2CC2CCCCCC)OCC2CC2CCCCCC)CCN(CCCCO)CC1)OCC1CC1CCCCCC] | CY68 |
| 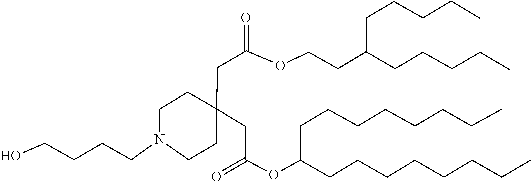 [SMILES: CCCCCCCCC(CCCCCCCC)OC(=O)CC1(CC(=O)OCCC(CCCCC)CCCCC)CCN(CCCCO)CC1] | CY69 |
| 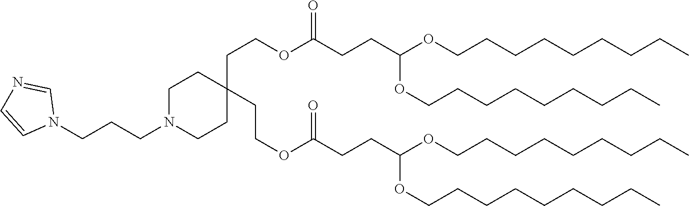 [SMILES: CCCCCCCCCOC(CCC(=O)OCCC1(CCOC(=O)CCC(OCCCCCCCCC)OCCCCCCCCC)CCN(CCCn2ccnc2)CC1)OCCCCCCCCC] | CY70 |
| 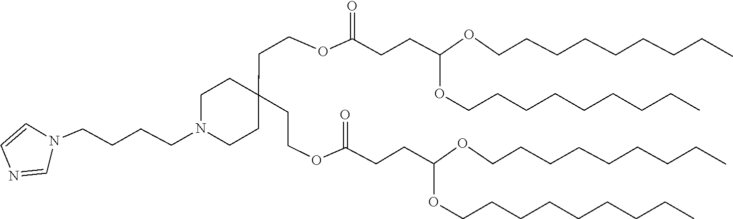 [SMILES: CCCCCCCCCOC(CCC(=O)OCCC1(CCOC(=O)CCC(OCCCCCCCCC)OCCCCCCCCC)CCN(CCCCn2ccnc2)CC1)OCCCCCCCCC] | CY71 |

In some embodiments, an LNP of the present disclosure comprises an ionizable lipid disclosed in PCT Application PCT/US2022/082276 (WO2023122752), which is incorporated by reference herein, in its entirety.

In one embodiment, the disclosure provides a compound of Formula IA:

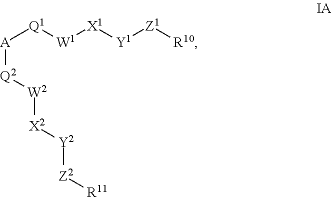

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

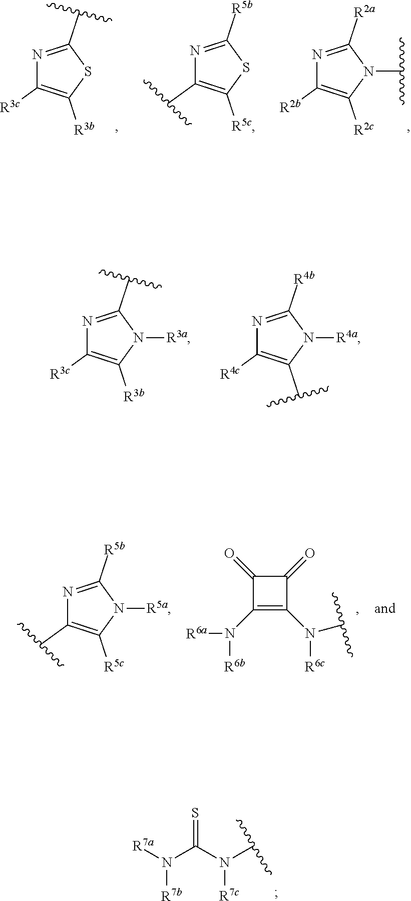

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

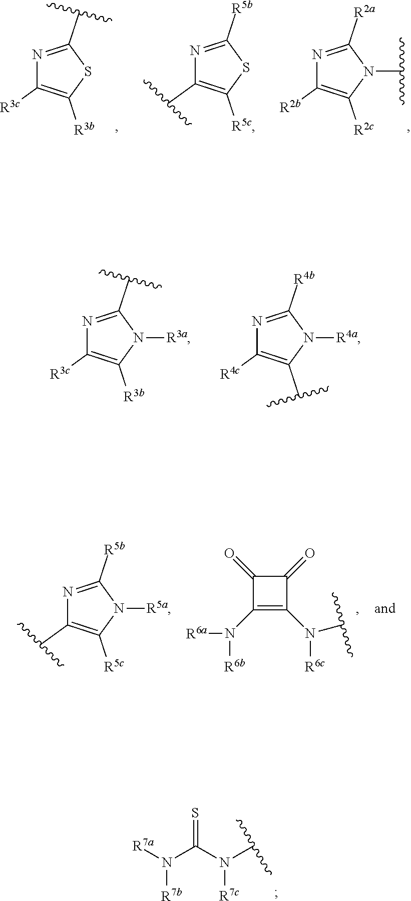

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂n alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₄-C₁₂ cycloalkylenyl,

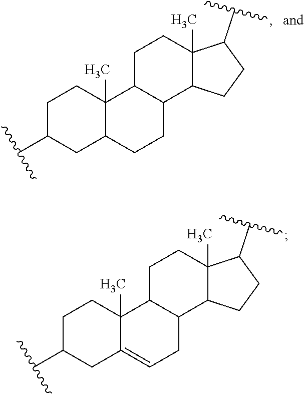 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X²is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl.

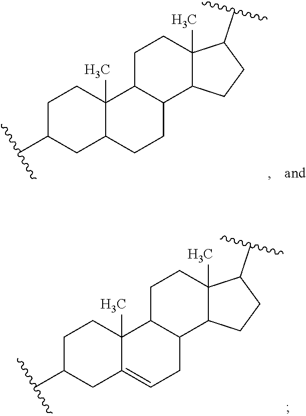 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IB:

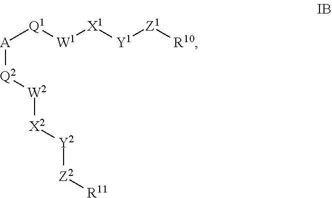

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

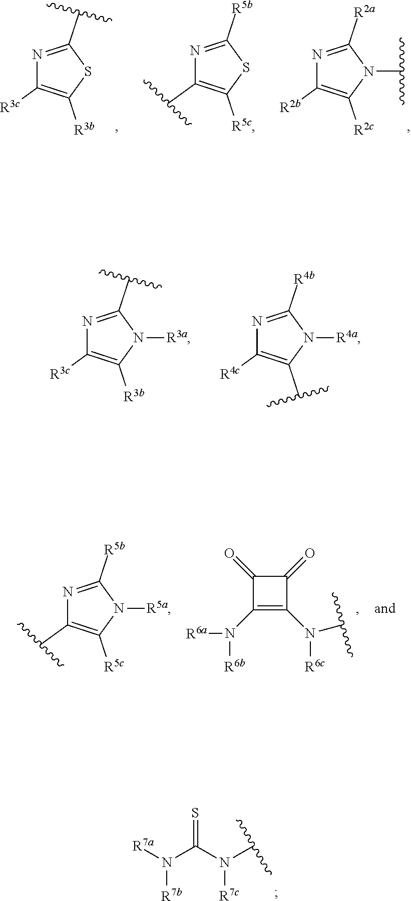

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl,
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

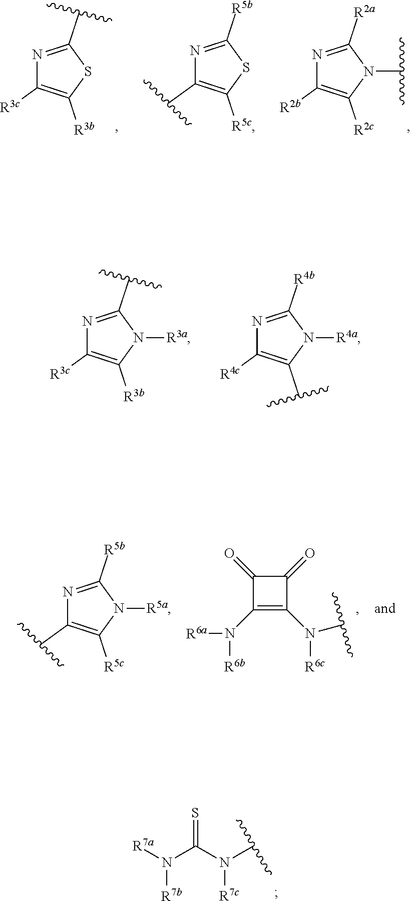

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₅-C₁₂ bridged cycloalkylenyl,

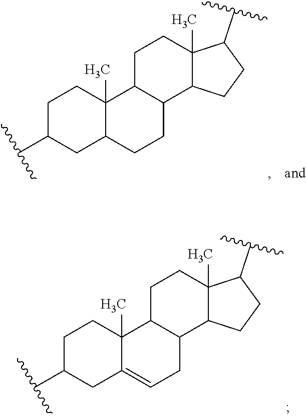 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X²is optionally substituted C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)ₚ—, optionally substituted C₄-C₁₂ cycloalkylenyl,

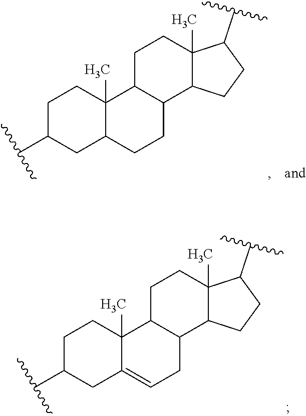 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R¹¹ is selected from the group consisting of hydrogen, C₁-C₁₀ alkyl, and C₂-C₁₀ alkenyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In one embodiment, the disclosure provides a compound of Formula IC:

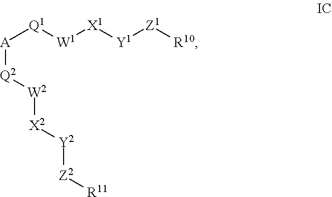

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—,
R¹ is selected from the group consisting of —OH,

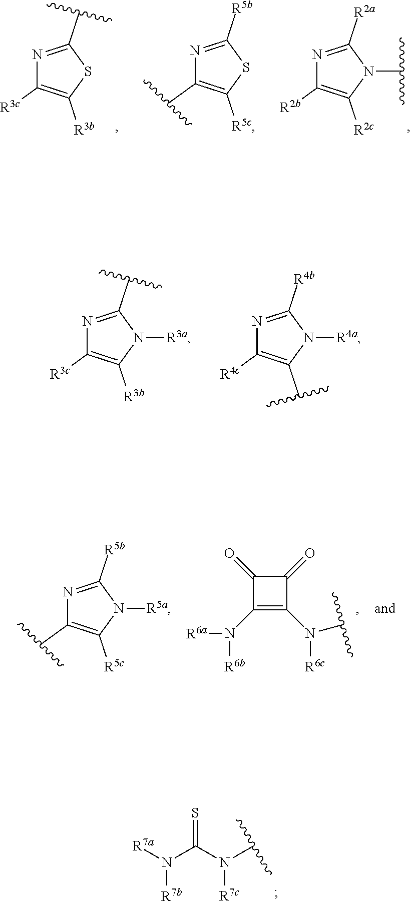

R^{2a}, R², and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₂₀ alkylenyl;
R⁸ is selected from the group consisting of —NRR^{9a}R^{9b},

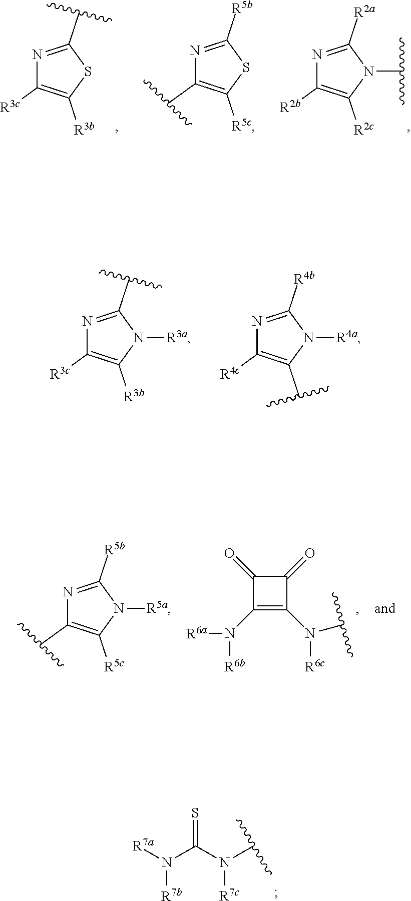

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{2a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is selected from the group consisting of optionally substituted C₄-C₁₂ cycloalkylenyl,

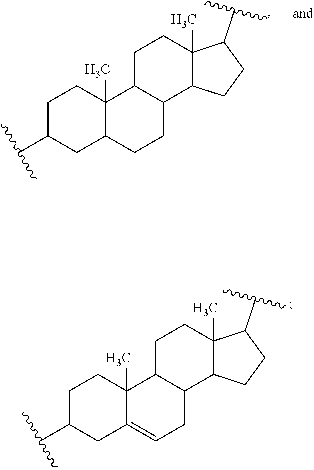 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is selected from the group consisting of —(CH₂)ₙ—, —O—, —S—, and —S—S—;
n is 0, 1, 2, 3, 4, 5, or 6;
Z² is of —(CH₂)ₚ—:
p is 0 or 1; and
R¹¹ is C₁-C₂₀ branched alkyl;
wherein one or more methylene linkages of X¹, X², Y¹, Y², Z¹, Z², R¹⁰, and R¹¹, are optionally and independently replaced with a group selected from —O—, —CH═CH—, —S— and C₃-C₆ cycloalkylenyl.

In some embodiments, the disclosure provides a compound of any one of Formulae IA, IB, IC, or a pharmaceutically acceptable salt or solvate thereof, wherein Z¹ is optionally substituted C₅-C₁₂ bridged cycloalkylenyl.

In some embodiments, the disclosure provides a compound of any one of Formulae IA, IB, IC, or a pharmaceutically acceptable salt or solvate thereof, wherein Z¹ is not adamantyl.

In one embodiment, the disclosure provides a compound of Formula ID:

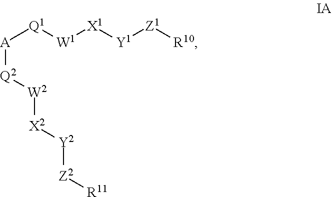

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl or —(CH₂)₂₋₆—OC(═O)—;
R¹ is selected from the group consisting of —OH,

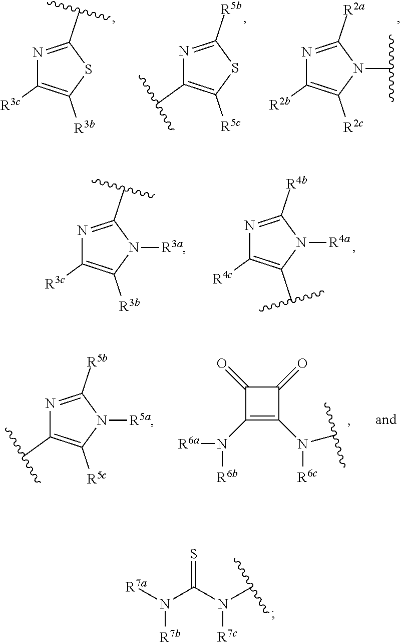

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is selected from the group consisting of —NR^{9a}R^{9b},

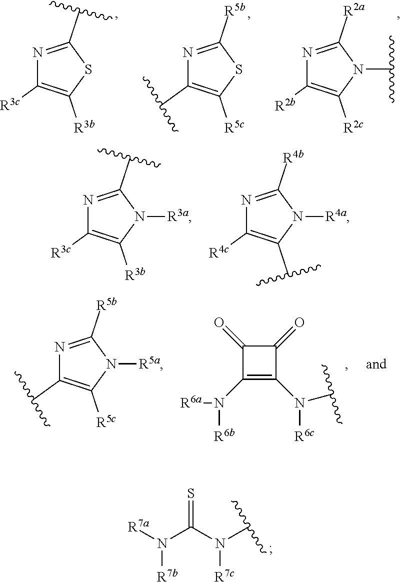

R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is optionally substituted branched C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6;
Z¹ is optionally substituted C₅-C₁₂ bridged cycloalkylenyl;
R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is optionally substituted C₁-C₁₅ alkylenyl; or
Y² is —(CH₂)ₙ—;
n is 0, 1, 2, 3, 4, 5, or 6;
Z² is of —(CH₂)ₚ—;
p is 0 or 1; and
R¹¹ is C₁-C₂₀ branched alkyl.

In some embodiments, the disclosure provides a compound of Formula ID or a pharmaceutically acceptable salt or solvate thereof, wherein Z¹ is not adamantyl.

In one embodiment, the disclosure provides a compound of Formula I:

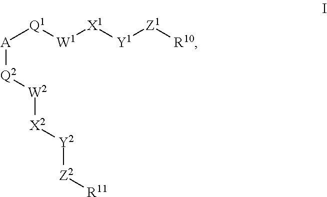

or a pharmaceutically acceptable salt or solvate thereof, wherein:
A is selected from the group consisting of —N(R^{1a})— and —C(R′)—OC(═O)(R^{8a})—;
R^{1a} is -L¹-R¹;
L¹ is C₂-C₆ alkylenyl;
R¹ is selected from the group consisting of —OH,

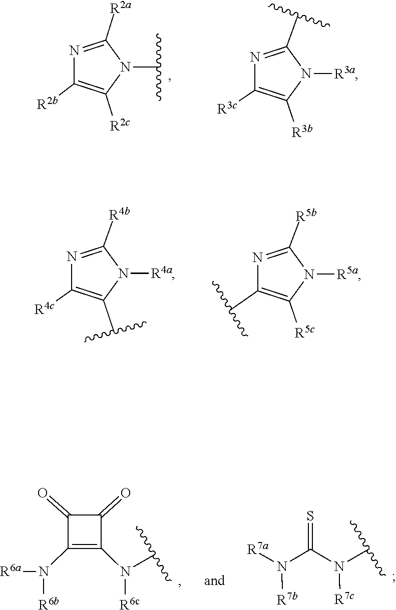

R^{2a}, R^{2b}, and R^{2c} are independently selected from the group consisting of hydrogen and C₁-C₆ alkyl;
R^{3a}, R^{3b}, and R^{3c} are independently selected from the group consisting of hydrogen and C₁-C₆ alkyl;
R^{4a}, R^{4b}, and R^{4c} are independently selected from the group consisting of hydrogen and C₁-C₆ alkyl;
R^{5a}, R^{5b}, and R^{5c} are independently selected from the group consisting of hydrogen and C₁-C₆ alkyl;
R^{6a}, R^{6b}, and R^{6c} are independently selected from the group consisting of hydrogen and C₁-C₆ alkyl; or
R^{6a} and R^{6b} taken together with the nitrogen atom to which they are attached form a 4- to 8-membered heterocyclo; and R^{6c} is selected from the group consisting of hydrogen and C₁-C₆ alkyl;
R^{7a}, R^{7b}, and R^{7c} are independently selected from the group consisting of hydrogen and C₁-C₆ alkyl; or
R^{7a} and R^{7b} taken together with the nitrogen atom to which they are attached form a 4- to 8-membered heterocyclo; and R^{7c} is selected from the group consisting of hydrogen and C₁-C₆ alkyl;
R′ is selected from the group consisting of hydrogen and C₁-C₆ alkyl;
R^{8a} is -L²-R⁸;
L² is C₂-C₆ alkylenyl;
R⁸ is —NR^{9a}R^{9b};
R^{9a} and R^{9b} are independently selected from the group consisting of hydrogen and C₁-C₆ alkyl; or
R^{9a} and R^{9b} taken together with the nitrogen atom to which they are attached form a 4- to 8-membered heterocyclo;
Q¹ is C₁-C₂₀ alkylenyl;
W¹ is selected from the group consisting of —C(═O)O—, —OC(═O)—, —C(═O)N(R^{2a})—, —(R^{12a})C(═O)—, —OC(═O)N(R^{12a})—, —N(R^{12a})C(═O)O—, and —OC(═O)O—;
R^{12a} is selected from the group consisting of hydrogen and C₁-C₆ alkyl;
X¹ is C₁-C₁₅ alkylenyl; or
X¹ is a bond;
Y¹ is selected from the group consisting of —(CH₂)ₘ—, —O—, —S—, and —S—S—;
m is 0, 1, 2, 3, 4, 5, or 6, Z¹ is selected from the group consisting of C₄-C₁₂ cycloalkylenyl,

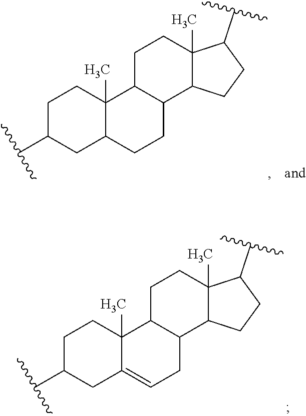 [SMILES: *C1CCC2(C)C(CCC3C4CCC(*)C4(C)CCC32)C1 ; *C1CCC2(C)C(=CCC3C2CCC2(C)C(*)CCC32)C1]

R¹⁰ is selected from the group consisting of hydrogen, C₁-C₂₀ alkyl, and C₂-C₂₀ alkenyl;
Q² is C₁-C₂₀ alkylenyl;
W² is selected from the group consisting of —C(═O)O—, —C(═O)N(R^{12b})—, —OC(═O)N(R^{12b})—, and —OC(═O)O—;
R^{12b} is selected from the group consisting of hydrogen and C₁-C₆ alkyl;
X² is C₁-C₁₅ alkylenyl; or
X² is a bond;
Y² is selected from the group consisting of —(CH₂)ₙ—, —O—, —S—, and —S—S—;
n is 0, 1, 2, 3, 4, 5, or 6;
Z² is selected from the group consisting of —(CH₂)—, C₄-C₁₂ cycloalkylenyl,

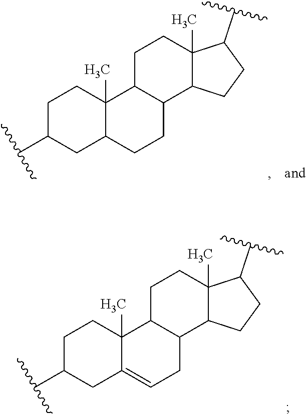 [SMILES: *C1CCC2(C)C(CCC3C4CCC(*)C4(C)CCC32)C1 ; *C1CCC2(C)C(=CCC3C2CCC2(C)C(*)CCC32)C1]

p is 0 or 1; and
R″ is selected from the group consisting of hydrogen, C₁-C₁₀ alkyl, and C₂-C₁₀ alkenyl.

In another embodiment, the disclosure provides a compound of Formula II:

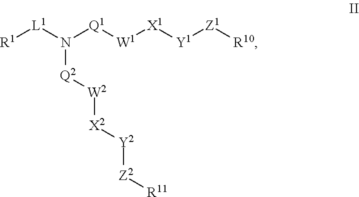

or a pharmaceutically acceptable salt or solvate thereof, wherein R¹, R¹⁰, R″, Q¹, Q², W¹, W², X¹, X², Y¹, Y², Z¹, and Z² are as defined herein in Formula IA, Formula IB, Formula IC, Formula ID, Formula I, or below

In another embodiment, the disclosure provides a compound of Formula III:

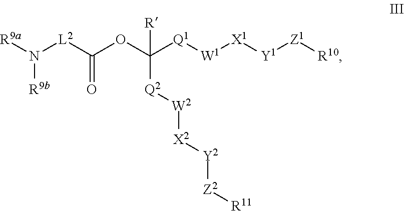

or a pharmaceutically acceptable salt or solvate thereof, wherein R′, R^{9a}, R^{9b}, R¹⁰, R¹¹, L², Q¹, Q², W¹, W², X¹, X², Y¹, Y², Z¹, and Z² are as defined herein in Formula IA, Formula IB, Formula IC, Formula ID, Formula I, or below.

In another embodiment, the disclosure provides a compound of Formula IV:

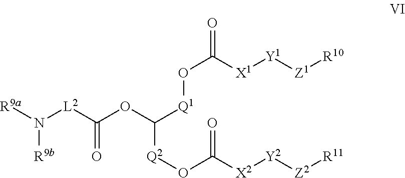

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below

In another embodiment, the disclosure provides a compound of Formula VI′:

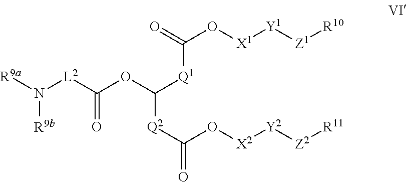

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below

In another embodiment, the disclosure provides a compound of Formula VI″:

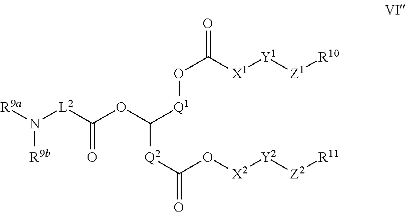

or a pharmaceutically acceptable salt or solvate thereof, wherein R^{9a}, R^{9b}, L², Q¹, Q², X¹, X², Y¹, Y², Z¹, Z², R¹⁰, an R¹¹ are as defined herein in Formula IA, Formula IB, Formula IC, Formula ID, Formula I, or below

In another embodiment, the disclosure provides a compound of Formula VI′″:

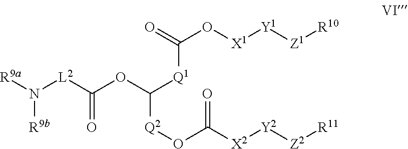

or a pharmaceutically acceptable salt or solvate thereof, wherein R^{9a}, R⁹, L², Q¹, Q², X², Y¹, Y², Z¹, Z², R¹⁰, an R¹¹ are as defined herein in Formula IA, Formula IB, Formula IC, Formula ID, Formula I, or below

In another embodiment, the disclosure provides a compound of Formula VII:

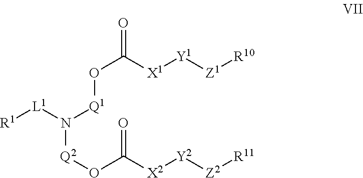

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VII′:

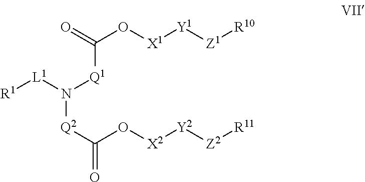

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VII″:

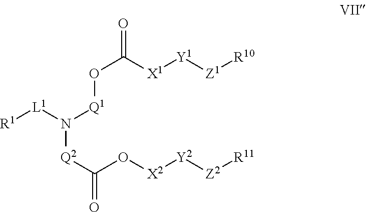

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula VII′″:

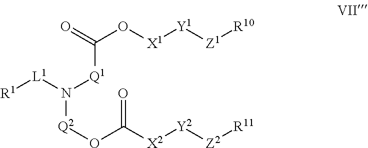

or a pharmaceutically acceptable salt or solvate thereof, wherein R¹, L¹, Q¹, Q², X¹, X², Y¹, Y², Z¹, Z², R¹⁰, an R¹¹ are as defined herein in Formula IA, Formula IB, Formula IC, Formula ID, Formula I, or below.

Formula IA, Formula IB, Formula IC, Formula I, In another embodiment, the disclosure provides a compound of Formula VIII:

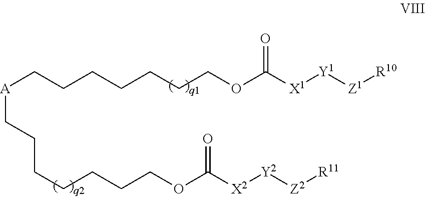

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII, wherein Z¹ is an optionally substituted C₅-C₁₂ bridged cycloalkylenyl.

In another embodiment, the disclosure provides a compound of Formula VIII′:

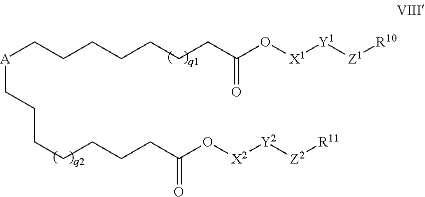

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII′, wherein Z¹ is an optionally substituted C₅-C₁₂ bridged cycloalkylenyl.

In another embodiment, the disclosure provides a compound of Formula VIII″:

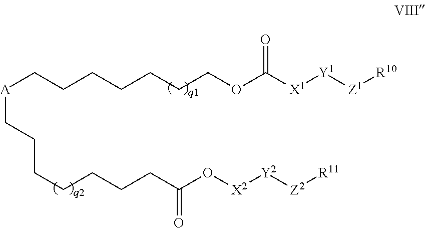

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII″, wherein Z¹ is an optionally substituted C₅-C₁₂ bridged cycloalkylenyl.

In another embodiment, the disclosure provides a compound of Formula VIII′″:

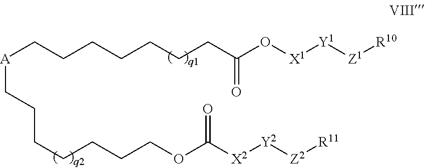

or a pharmaceutically acceptable salt or solvate thereof,
wherein
q¹ is 0, 1, 2, or 3:
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula VIII′″, wherein Z¹ is an optionally substituted C₅-C₁₂ bridged cycloalkylenyl.

In another embodiment, the disclosure provides a compound of Formula IX:

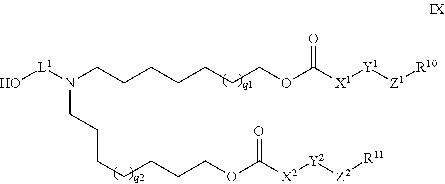

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX, wherein Z¹ is an optionally substituted C₅-C₁₂ bridged cycloalkylenyl.

In another embodiment, the disclosure provides a compound of Formula IX′:

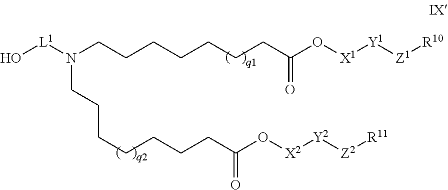

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX′, wherein Z¹ is an optionally substituted C₅-C₁₂ bridged cycloalkylenyl.

In another embodiment, the disclosure provides a compound of Formula IX″:

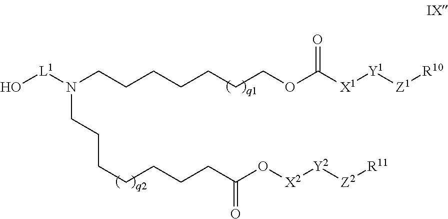

or a pharmaceutically acceptable salt or solvate thereof,
wherein
q¹ is 0, 1, 2, or 3;
q² is 0, 1, 2, or 3:
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX″, wherein Z¹ is an optionally substituted C₅-C₁₂ bridged cycloalkylenyl.

In another embodiment, the disclosure provides a compound of Formula IX′″:

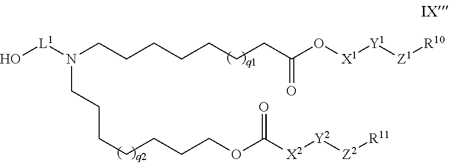

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula IX′″, wherein Z¹ is an optionally substituted C₅-C₁₂ bridged cycloalkylenyl.

In another embodiment, the disclosure provides a compound of Formula X:

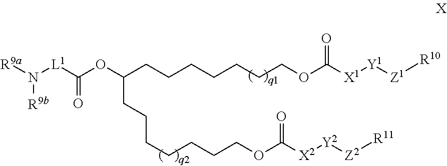

or a pharmaceutically acceptable salt or solvate thereof,
wherein
q¹ is 0, 1, 2, or 3,
q² is 0, 1, 2, or 3;
L¹, X¹, X², Y¹, Y², Z¹, Z², R^{9a}, R^{9b}, R¹⁰, an R¹¹ are as defined herein in Formula IA, Formula IB, Formula IC, Formula ID, Formula I or below.

In certain embodiments, the compound is a compound of Formula X, wherein Z¹ is an optionally substituted C₅-C₁₂ bridged cycloalkylenyl.

In another embodiment, the disclosure provides a compound of Formula X′:

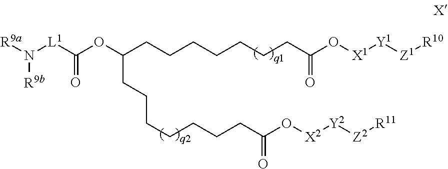

or a pharmaceutically acceptable salt or solvate thereof,
wherein
q¹ is 0, 1, 2, or 3:
q² is 0, 1, 2, or 3;
L¹, X¹, X², Y¹, Y², Z¹, Z², R^{9a}, R^{9b}, R¹⁰, an R¹¹ are as defined herein in Formula IA, Formula IB, Formula IC, Formula ID, Formula I or below.

In certain embodiments, the compound is a compound of Formula X′, wherein Z¹ is an optionally substituted C₅-C₁₂ bridged cycloalkylenyl.

In another embodiment, the disclosure provides a compound of Formula X″:

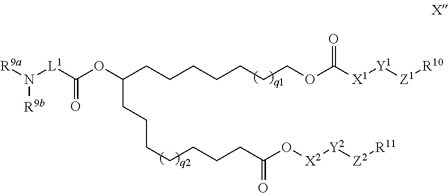

or a pharmaceutically acceptable salt or solvate thereof,
wherein
q¹ is 0, 1, 2, or 3;
q² is 0, 1, 2, or 3;
L¹, X¹, X², Y¹, Y², Z¹, Z², R^{9a}, R^{9b}, R¹⁰, an R¹¹ are as defined herein in Formula IA, Formula IB, Formula IC, Formula ID, Formula I or below.

In certain embodiments, the compound is a compound of Formula X″, wherein Z¹ is an optionally substituted C₅-C₁₂ bridged cycloalkylenyl.

In another embodiment, the disclosure provides a compound of Formula X′″:

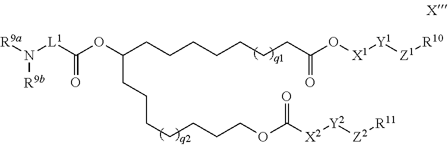

or a pharmaceutically acceptable salt or solvate thereof,
wherein
q¹ is 0, 1, 2, or 3:
q² is 0, 1, 2, or 3;
L¹, X¹, X², Y¹, Y², Z¹, Z², R^{9a}, R %, R¹⁰, an R¹¹ are as defined herein in Formula IA, Formula IB, Formula IC, Formula ID, Formula I or below.

In certain embodiments, the compound is a compound of Formula X′″, wherein Z¹ is an optionally substituted C₅-C₁₂ bridged cycloalkylenyl.

In another embodiment, the disclosure provides a compound of Formula XI:

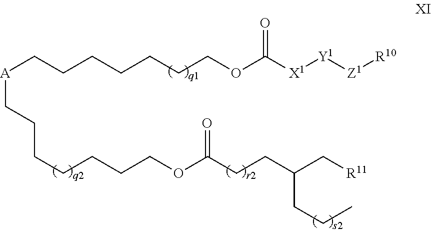

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI, wherein Z¹ is an optionally substituted C₅-C₁₂ bridged cycloalkylenyl.

In another embodiment, the disclosure provides a compound of Formula XI′:

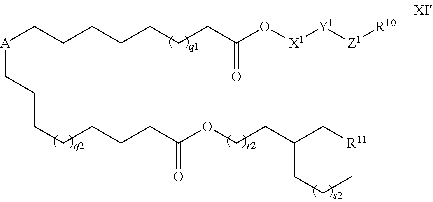

or a pharmaceutically acceptable salt or solvate thereof,
wherein
q¹ is 0, 1, 2, or 3:
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI′, wherein Z¹ is an optionally substituted C₅-C₁₂ bridged cycloalkylenyl.

In another embodiment, the disclosure provides a compound of Formula XI″:

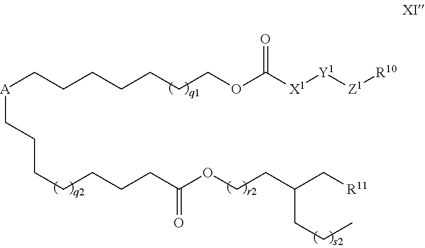

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI″, wherein Z¹ is an optionally substituted C₅-C₁₂ bridged cycloalkylenyl.

In another embodiment, the disclosure provides a compound of Formula XI′″:

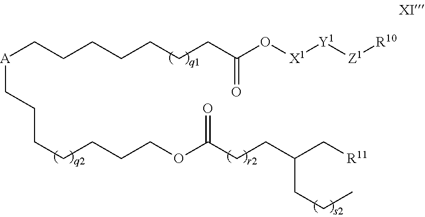

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XI′″, wherein Z¹ is an optionally substituted C₅-C₁₂ bridged cycloalkylenyl.

In another embodiment, the disclosure provides a compound of Formula XII:

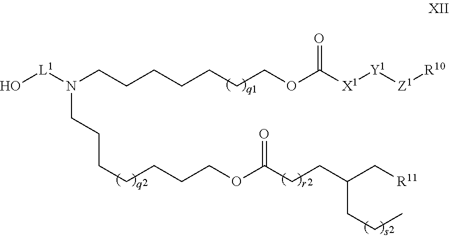

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I, or below.

In certain embodiments, the compound is a compound of Formula XII, wherein Z¹ is an optionally substituted C₅-C₁₂ bridged cycloalkylenyl.

In another embodiment, the disclosure provides a compound of Formula XII′:

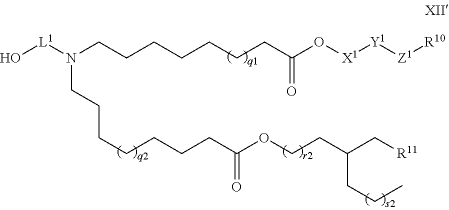

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I, or below.

In certain embodiments, the compound is a compound of Formula XII′, wherein Z¹ is an optionally substituted C₅-C₁₂ bridged cycloalkylenyl.

In another embodiment, the disclosure provides a compound of Formula XII″:

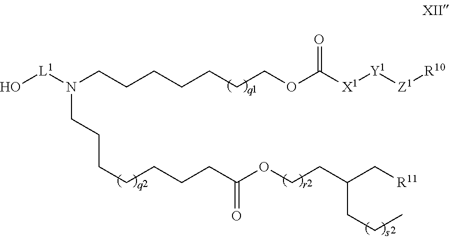

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I or below.

In certain embodiments, the compound is a compound of Formula XII″, wherein Z¹ is an optionally substituted C₅-C₁₂ bridged cycloalkylenyl.

In another embodiment, the disclosure provides a compound of Formula XII′″:

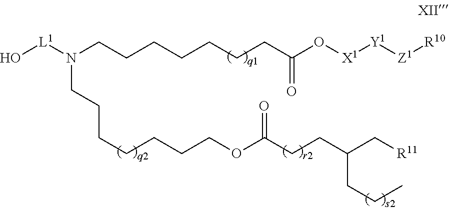

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula IC, Formula ID, Formula I or below.

In certain embodiments, the compound is a compound of Formula XII′″, wherein Z¹ is an optionally substituted C₅-C₂ bridged cycloalkylenyl.

In another embodiment, the disclosure provides a compound of Formula XIII:

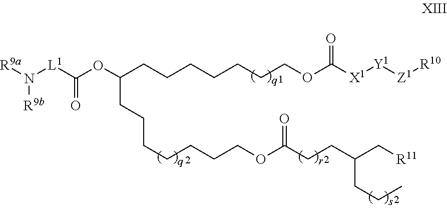

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula I or below.

In certain embodiments, the compound is a compound of Formula XIII, wherein Z¹ is an optionally substituted C₅-C₁₂ bridged cycloalkylenyl.

In another embodiment, the disclosure provides a compound of Formula XIII′:

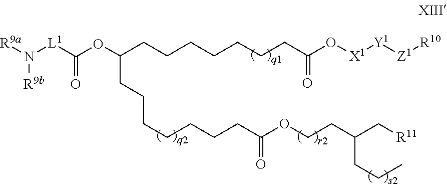

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In certain embodiments, the compound is a compound of Formula XIII′, wherein Z¹ is an optionally substituted C₅-C₁₂ bridged cycloalkylenyl.

In another embodiment, the disclosure provides a compound of Formula XIII″:

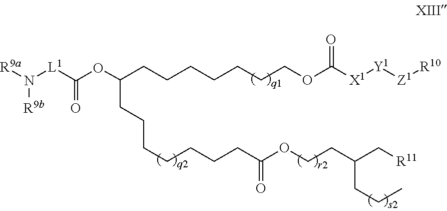

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In certain embodiments, the compound is a compound of Formula XIII″, wherein Z¹ is an optionally substituted C₅-C₁₂ bridged cycloalkylenyl.

In another embodiment, the disclosure provides a compound of Formula XIII′″:

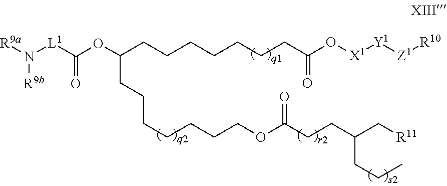

or a pharmaceutically acceptable salt or solvate thereof,
wherein
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I or below.

In certain embodiments, the compound is a compound of Formula XIII′″, wherein Z¹ is an optionally substituted C₅-C₁₂ bridged cycloalkylenyl.

In another embodiment, the disclosure provides a compound of Formula XIV:

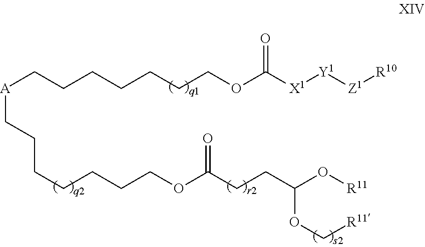

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XIV, wherein Z¹ is an optionally substituted C₅-C₁₂ bridged cycloalkylenyl. In certain embodiments, Z¹ is not adamantyl.

In another embodiment, the disclosure provides a compound of Formula XIV′:

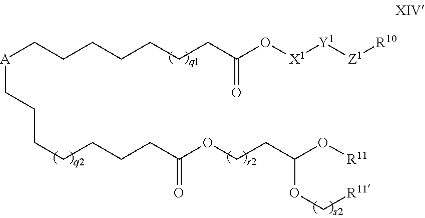

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I, or below.

In certain embodiments, the compound is a compound of Formula XIV′, wherein Z¹ is an optionally substituted C₅-C₁₂ bridged cycloalkylenyl. In certain embodiments, Z¹ is not adamantyl.

In another embodiment, the disclosure provides a compound of Formula XIV″:

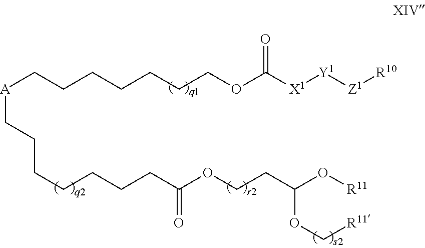

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I or below.

In certain embodiments, the compound is a compound of Formula XIV″, wherein Z¹ is an optionally substituted C₅-C₁₂ bridged cycloalkylenyl. In certain embodiments, Z¹ is not adamantyl.

In another embodiment, the disclosure provides a compound of Formula XIV′″:

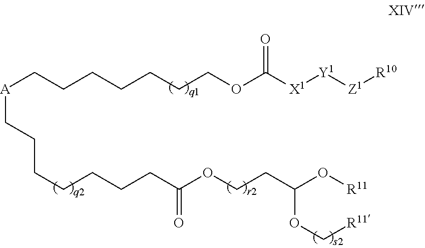

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
A, X¹, Y¹, Z¹, R¹⁰, and R¹¹ are as defined herein in Formula IA, Formula IB, Formula IC, Formula ID, Formula I or below.

In certain embodiments, the compound is a compound of Formula XIV′″, wherein Z¹ is an optionally substituted C₅-C₂ bridged cycloalkylenyl. In certain embodiments, Z¹ is not adamantyl.

In another embodiment, the disclosure provides a compound of Formula XV:

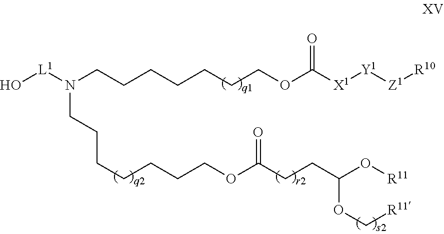

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV′:

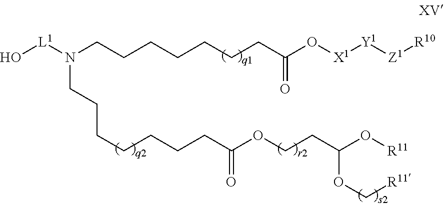

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV″:

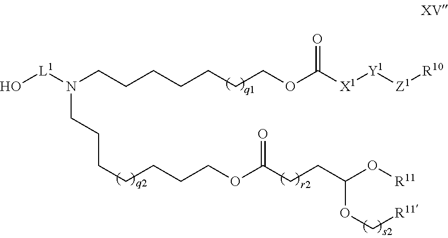

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XV′″:

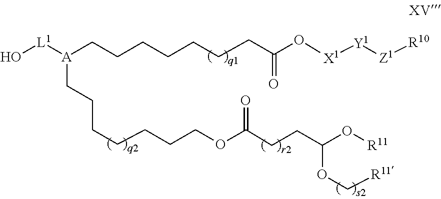

or a pharmaceutically acceptable salt or solvate thereof,
wherein
R^{11′} is selected from the group consisting of hydrogen, C₁-C₁₀ alkyl, and C₂-C₁₀ alkenyl; q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R¹⁰ and R¹¹ are as defined herein in Formula IA, Formula IB, Formula, IC, Formula I or below;
wherein Z¹ is not adamantyl.

In another embodiment, the disclosure provides a compound of Formula XVI:

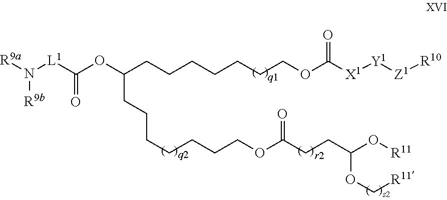

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI′:

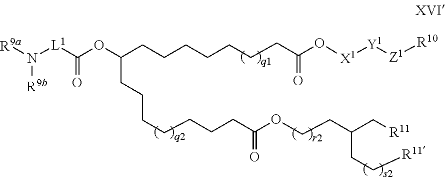

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI″:

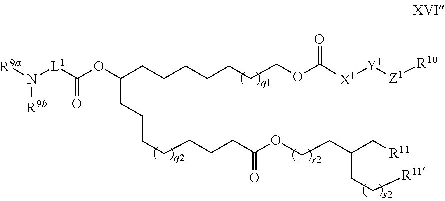

or a pharmaceutically acceptable salt or solvate thereof,
wherein
R^{11′} is selected from the group consisting of hydrogen. C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVI′″:

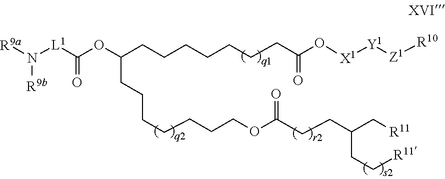

or a pharmaceutically acceptable salt or solvate thereof,
wherein
R^{11′} is selected from the group consisting of hydrogen, C₁-C₁₀ alkyl, and C₂-C₁₀ alkenyl;
q¹ is 0, 1, 2, or 3;
q² is 0, 1, 2, or 3;
r² is 0, 1, or 2;
s² is 0, 1, 2, 3, 4, 5, 6; and
L¹, X¹, Y¹, Z¹, R^{9a}, R^{9b}, R¹⁰ and R¹¹ are as defined herein in Formula IA, Formula IB, Formula IC, Formula ID, Formula I, or below.

In another embodiment, the disclosure provides a compound of Formula XVII:

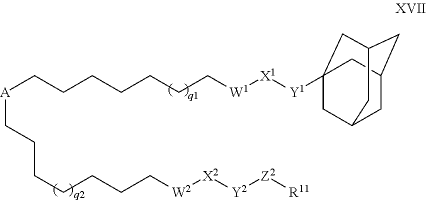

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In certain embodiments, the compound is a compound of Formula XVII, wherein one or more methylene linkages of X², Y², Z², and R¹¹, are not replaced with a group selected from —O—, —CH═CH—, —S— and C₃-C₆ cycloalkylenyl.

In another embodiment, the disclosure provides a compound of Formula XVIII:

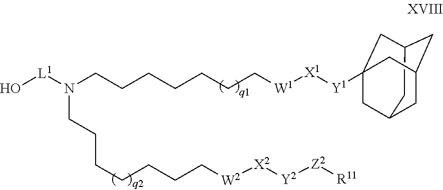

or a pharmaceutically acceptable salt or solvate thereof,
wherein
q¹ is 0, 1, 2, or 3;
q² is 0, 1, 2, or 3;
L¹, X¹, X², Y¹, Y², Z², an R¹¹ are as defined herein in Formula IA, Formula IB, Formula IC, Formula ID, Formula I, or below.

In certain embodiments, the compound is a compound of Formula XVIII, wherein one or more methylene linkages of X², Y², Z², and R″, are not replaced with a group selected from —O—, —CH═CH—, —S— and C₃-C₆ cycloalkylenyl.

In another embodiment, the disclosure provides a compound of Formula XVIII′:

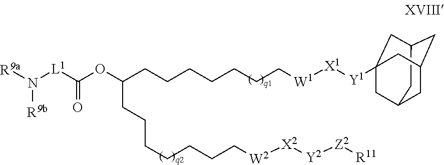

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA. Formula IB, Formula IC, Formula ID, Formula I, or below.

In another embodiment, the disclosure provides a compound of Formula XIX:

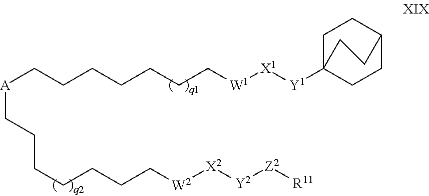

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

In another embodiment, the disclosure provides a compound of Formula XX:

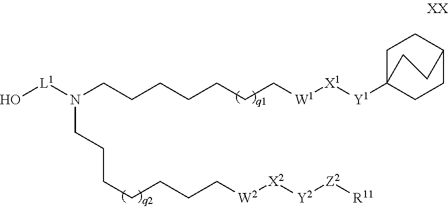

or a pharmaceutically acceptable salt or solvate thereof,
wherein
q¹ is 0, 1, 2, or 3;
q² is 0, 1, 2, or 3:
L¹, X¹, X², Y¹, Y², Z², an R¹¹ are as defined herein in Formula IA, Formula IB, Formula IC, Formula ID, Formula I, or below.

In another embodiment, the disclosure provides a compound of Formula XXI:

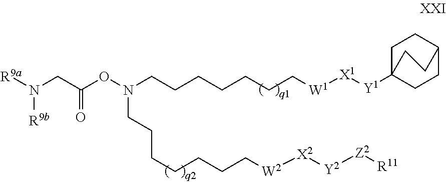

or a pharmaceutically acceptable salt or solvate thereof,
wherein
q¹ is 0, 1, 2, or 3;
q² is 0, 1, 2, or 3;
A, X¹, X², Y¹, Y², Z¹, Z², R¹⁰, an R¹¹ are as defined herein in Formula IA, Formula IB, Formula IC, Formula ID, Formula I, or below.

### L¹

In another embodiment, L1 is selected from the group consisting of —CH2CH2-, —CH2CH2CH2-, and —CH2CH2CH2CH2-. In another embodiment, L1 is —CH2CH2-. In another embodiment, L1 is —CH2CH2CH2-. In another embodiment, L¹ is —CH₂CH₂CH₂CH₂—. In certain embodiments, L¹ is —(CH₂)₂₋₆—OC(═O)—. In some embodiments, L¹ is —(CH₂)₂—OC(═O)—.

### R¹

In some embodiments, R¹ is

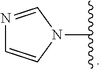 [SMILES: *n1ccnc1]

In another embodiment, R¹ is —OH. In some embodiments, R¹ is —N(R^{9a})(R^{9b}). In some embodiments, R¹ is —NMe₂. In some embodiments, R¹ is —NEt₂. In another embodiment, R¹ is

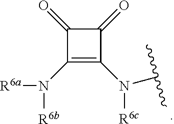

In another embodiment, R¹ is

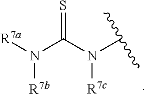

### L²

In another embodiment, L² is selected from the group consisting of —CH₂CH₂—, —CH₂CH₂CH₂—, and —CH₂CH₂CH₂CH₂—. In another embodiment, L² is —CH₂CH₂—. In another embodiment, L² is —CH₂CH₂CH₂—. In another embodiment, L² is —CH₂CH₂CH₂CH₂—.

### R⁸

In some embodiments, R⁸ is

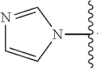 [SMILES: *n1ccnc1]

In another embodiment, R⁸ is —NR^{9a}R^{9b}. In some embodiments, R⁸ is —NMe₂. In some embodiments, R⁸ is —NEt₂. In another embodiment, R⁸ is —OH.
R^{9a}, R^{9b}

In another embodiment, R^{9a} and R^{9b} are independently selected from the group consisting of hydrogen and C₁-C₄ alkyl. In another embodiment, R^{9a} and R^{9b} are each methyl. In another embodiment, R^{9a} and R^{9b} are each ethyl.

### R′

In another embodiment, R′ is hydrogen. In some embodiments, R′ is C₁-C₆ alkyl.

In another embodiment, Q¹ is straight chain C₁-C₂₀ alkylenyl. In another embodiment, Q¹ is straight chain C₁-C₁₀ alkylenyl. In another embodiment, Q¹ is C₁-C₁₀ alkylenyl. In another embodiment, Q¹ is C₂-C₅ alkylenyl. Q¹ is C₆-C₉ alkylenyl. In another embodiment, Q¹ is selected from the group consisting of —CH₂CH₂—, —CH₂CH₂CH₂—, —CH₂(CH₂)₂CH₂—, —CH₂(CH₂)₃CH₂—, —CH₂(CH₂)₄CH₂—, —CH₂(CH₂)₅CH₂—, —CH₂(CH₂)₆CH₂—, —CH₂(CH₂)₇CH₂—, and —CH₂(CH₂)₈CH₂—. In another embodiment, Q¹ is —CH₂CH₂—. In another embodiment, Q¹ is —CH₂CH₂CH₂—. In another embodiment, Q¹ is —CH₂(CH₂)₂CH₂—. In another embodiment, Q¹ is —CH₂(CH₂)₃CH₂—. In another embodiment, Q¹ is —CH₂CH₂—. In another embodiment, Q¹ is —CH₂(CH₂)₄CH₂—. In another embodiment, Q¹ is —CH₂(CH₂)₅CH₂—. In another embodiment, Q¹ is —CH₂(CH₂)₆CH₂—. In another embodiment, Q¹ is —CH₂(CH₂)₇CH₂—. In another embodiment, Q¹ is —CH₂(CH₂)₈CH₂—.

### W¹

In another embodiment, W¹ is selected from the group consisting of —C(═O)O—, —OC(═O)—, —C(═O)N(R^{12a})—, —N(R^{12a})C(═O)—, —OC(═O)N(R^{12a})—, —N(R^{12a})C(═O)O—, and —OC(═O)O—. In another embodiment, W¹ is —C(═O)O—. In another embodiment, W¹ is —OC(═O)—. In another embodiment, W¹ is —C(═O)N(R^{12a})—. In another embodiment, W¹ is —N(R^{12a})C(═O)—. In another embodiment, W¹ is —OC(═O)N(R^{12a})—. In another embodiment, W¹ is —N(R^{12a})C(═O)O—. In another embodiment, W¹ is —OC(═O)O—.

### X¹

In another embodiment, X²is optionally substituted C₁-C₁₅ alkylenyl. In another embodiment, X²is branched C₁-C₁₅ alkylenyl. In another embodiment, X¹ is a bond or C₁-C₁₅ alkylenyl. In another embodiment, X¹ is a bond. In another embodiment, X is C₂-C₅ alkylenyl. In another embodiment, X¹ is C₆-C₉ alkylenyl. In another embodiment, X¹ is —CH₂—. In another embodiment, X² is —CH₂CH₂—. In another embodiment, X² is —CH₂CH₂CH₂—. In another embodiment, X² is —CH₂CH₂CH₂CH₂—. In another embodiment, X² is —CH₂CH₂CH₂CH₂CH₂—.

### Y¹

In another embodiment, Y¹ is selected from the group consisting of —(CH₂)ₘ—, —O—, —S—, and —S—S—. In another embodiment, Y¹ is —(CH₂)ₘ—. In some embodiments, Y¹ is —O—. In some embodiments, Y¹ is —S—. In another embodiment, Y¹ is —CH₂—. In another embodiment, Y² is —CH₂CH₂—.

m

In another embodiment, m is 0. In another embodiment, m is 1. In another embodiment, m is 2. In another embodiment, m is 3. In another embodiment, m is 4. In another embodiment, m is 5. In another embodiment, m is 6.

n

In another embodiment, n is 0. In another embodiment, n is 1. In another embodiment, n is 2. In another embodiment, n is 3. In another embodiment, n is 4. In another embodiment, n is 5. In another embodiment, n is 6.

### P

In another embodiment, p is 0. In another embodiment, p is 1.

### Z¹

In another embodiment, Z¹ is selected from the group consisting of C₄-C₁₂ cycloalkylenyl,

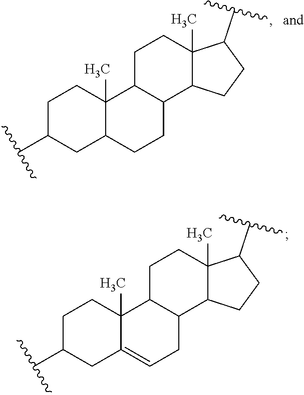 [SMILES: *C1CCC2(C)C(CCC3C4CCC(*)C4(C)CCC32)C1 ; *C1CCC2(C)C(=CCC3C2CCC2(C)C(*)CCC32)C1]

In certain embodiments, Z¹ is optionally substituted.

In another embodiment, Z¹ is

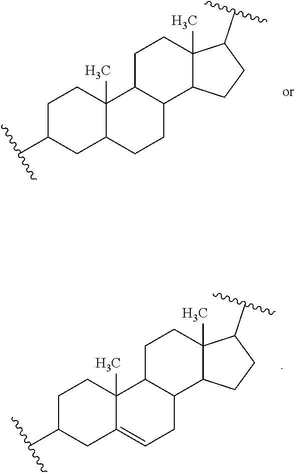 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, Z¹ is C₄-C₁₂ cycloalkylenyl. In another embodiment, Z¹ is a monocyclic C₄-C₈ cycloalkylenyl. In another embodiment, Z¹ is a monocyclic C₄-C₆ cycloalkylenyl. In another embodiment, Z¹ is a monocyclic C₄ cycloalkylenyl. In another embodiment, Z¹ is a monocyclic C₅ cycloalkylenyl. In another embodiment, Z¹ is a monocyclic C₆ cycloalkylenyl.

In another embodiment, Z¹ is an optionally substituted bridged bicyclic or multicyclic cycloalkylenyl. In some embodiments, Z¹ is optionally substituted C₅-C₁₂ bridged cycloalkylenyl. In some embodiments, Z¹ is optionally substituted C₆-C₁₀ bridged cycloalkylenyl. In some embodiments, Z¹ is a optionally substituted C₅-C₁₀ bridged cycloalkylenyl. selected from the group consisting of adamantyl, cubanyl, bicyclo[2.2.1]heptyl, bicyclo[2.2.2]octyl, bicyclo[1.1.1]pentyl, bicyclo[3.2.1]octyl, and bicyclo[3.1.1]heptyl.

In another embodiment, Z¹ is selected from the group consisting of:

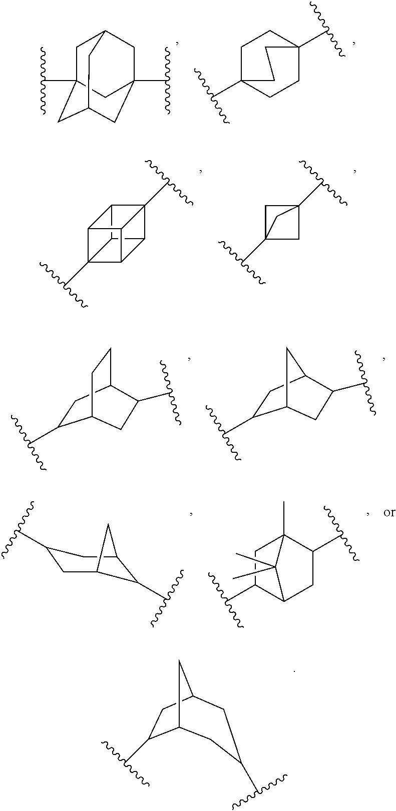

In another embodiment, Z¹ is selected from the group consisting of:

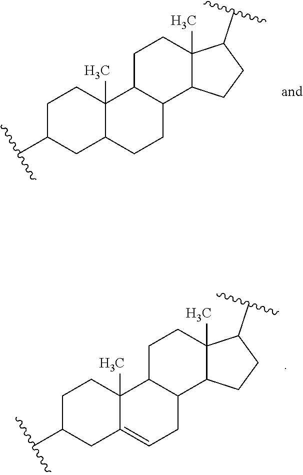 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, R¹⁰ is hydrogen.

In another embodiment, R¹⁰ is C₁-C₁₀ alkyl. In another embodiment, R¹⁰ is C₃-C₇ alkyl. In another embodiment, R¹⁰ is C₄-C₆ alkyl. In another embodiment, R¹⁰ is C₄. In another embodiment, R¹⁰ is C₅. In another embodiment, R¹⁰ is C₆.

In another embodiment, R¹⁰ is C₂-C₁₂ alkenyl. In another embodiment, R¹⁰ is C₆-C₁₂ alkenyl. In another embodiment, R¹⁰ is C₂-C₈ alkenyl.

### R¹¹

In another embodiment, R¹¹ is C₁-C₁₀ alkyl. In another embodiment, R¹¹ is optionally substituted C₁-C₂₀ alkyl. In another embodiment, R¹¹ is optionally substituted branched C₁-C₂₀ alkyl. In another embodiment, R¹¹ is optionally substituted C₁-C₁₅ alkyl. In another embodiment, R¹¹ is optionally substituted C₁-C₁₅ branched alkyl. In another embodiment, R¹¹ is optionally substituted C₁₀-C₁₅ alkyl. In another embodiment, R¹¹ is optionally substituted C₁₀-C₁₅ branched alkyl. In another embodiment, R¹¹ is selected from the group consisting of —CH₃, —CH₂CH₃, and —CH₂CH₂CH₃. In another embodiment, R¹¹ is selected from the group consisting of —CH₂(CH₂)₂CH₃, —CH₂(CH₂)₃CH₃, —CH₂(CH₂)₄CH₃, —CH₂(CH₂)₅CH₃, —CH₂(CH₂)₆CH₃, —CH₂(CH₂)₇CH₃, and —CH₂(CH₂)₈CH₃. In another embodiment, R¹¹ is —CH₃. In another embodiment, R¹¹ is —CH₂CH₃. In another embodiment, R¹¹ is —CH₂CH₂CH₃. In another embodiment, R¹¹ is —CH₂(CH₂)₂CH₃. In another embodiment, R¹¹ is —CH₂(CH₂)₃CH₃. In another embodiment, R¹¹ is —CH₂(CH₂)₄CH₃. In another embodiment, R¹¹ is —CH₂(CH₂)₅CH₃. In another embodiment, R¹¹ is CH₂(CH₂)₆CH₃. In another embodiment, R¹¹ is —CH₂(CH₂)₇CH₃. In another embodiment, R¹¹ is —CH₂(CH₂)₈CH₃.

In another embodiment, R¹¹ is C₂-C₁₀ alkenyl. In another embodiment, R¹¹ is C₂-C₁₂ alkenyl. In another embodiment, R¹¹ is C₆-C₁₂ alkenyl. In another embodiment, R¹¹ is C₂-C₈ alkenyl.

In another embodiment, the disclosure provides a compound of any one of Formulae IA, IB, IC, or I-XXI or a pharmaceutically acceptable salt or solvate thereof, wherein R¹¹ is hydrogen.

### Q²

In another embodiment, Q² is straight chain C₁-C₂₀ alkylenyl. In another embodiment, Q² is straight chain C₁-C₁₀ alkylenyl. In another embodiment, Q² is C₂-C₁₀ alkylenyl. In another embodiment, Q² is selected from the group consisting of —CH₂CH₂—, —CH₂CH₂CH₂—, —CH₂(CH₂)₂CH₂—, —CH₂(CH₂)₃CH₂—, —CH₂(CH₂)₄CH₂—, —CH₂(CH₂)₅CH₂—, —CH₂(CH₂)₆CH₂—, —CH₂(CH₂)₇CH₂—, and —CH₂(CH₂)₈.CH₂—. In another embodiment, Q² is —CH₂CH₂—. In another embodiment, Q² is —CH₂CH₂CH₂—. In another embodiment, Q² is —CH₂(CH₂)₃CH₂—. In another embodiment, Q² is —CH₂(CH₂)₄CH₂—. In another embodiment, Q² is —CH₂(CH₂)₅CH₂—. In another embodiment, Q² is —CH₂(CH₂)₆CH₂—. In another embodiment, Q² is —CH₂(CH₂)₇CH₂—. In another embodiment, Q² is —CH₂(CH₂)₈CH₂—.

### W²

In another embodiment, W² is selected from the group consisting of —C(═O)O— and —OC(═O)—. In another embodiment, W² is —C(═O)O—. In another embodiment, W² is —OC(═O)—.

### X²

In another embodiment, X²is optionally substituted C₁-C₁₅ alkylenyl. In another embodiment, X²is C₁-C₁₅ branched alkylenyl. In another embodiment, X² is C₁-C₆ alkylenyl or a bond. In another embodiment, X² is C₂-C₄ alkylenyl. In another embodiment, X² is C₃-C₅ alkylenyl. In another embodiment, X² is selected from the group consisting of —CH₂CH₂—, —CH₂CH₂CH₂—, —CH₂(CH₂)₂CH₂—, —CH₂(CH₂)₃CH₂—, and —CH₂(CH₂)₄CH₂—. In another embodiment, X² is —CH₂—. In another embodiment, X² is a bond. In another embodiment, X² is branched C₁-C₁₅ alkylenyl, wherein one or more methylene linkages of X² are optionally and independently replaced with a group selected from —O—, —CH═CH—, —S— and C₃-C₆ cycloalkylenyl.

### Y²

In another embodiment, Y² is selected from the group consisting of —(CH₂)ₘ— and —S—. In another embodiment, Y² is —(CH₂)ₘ—. In another embodiment, Y² is —S—.

### Z²

In another embodiment, Z² is —(CH₂)ₚ—. In another embodiment, Z² is —CH₂—. In another embodiment, Z² is —CH₂CH₂—. In another embodiment, Z² is C₄-C₁₂ cycloalkylenyl. In another embodiment, Z² is a monocyclic C₄-C₈ cycloalkylenyl. In certain embodiments, Z² is optionally substituted.

In another embodiment, Z² is an optionally substituted bridged bicyclic or multicyclic cycloalkylenyl. In some embodiments, Z² is optionally substituted C₅-C₁₂ bridged cycloalkylenyl. In some embodiments, Z² is optionally substituted C₆-C₁₀ bridged cycloalkylenyl. In some embodiments, Z² is a optionally substituted C₅-C₁₀ bridged cycloalkylenyl. selected from the group consisting of adamantyl, cubanyl, bicyclo[2.2.1]heptyl, bicyclo[2.2.2]octyl, bicyclo[1.1.1]pentyl, bicyclo[3.2.1]octyl, and bicyclo[3.1.1]heptyl.

In another embodiment, Z² is selected from the group consisting of:

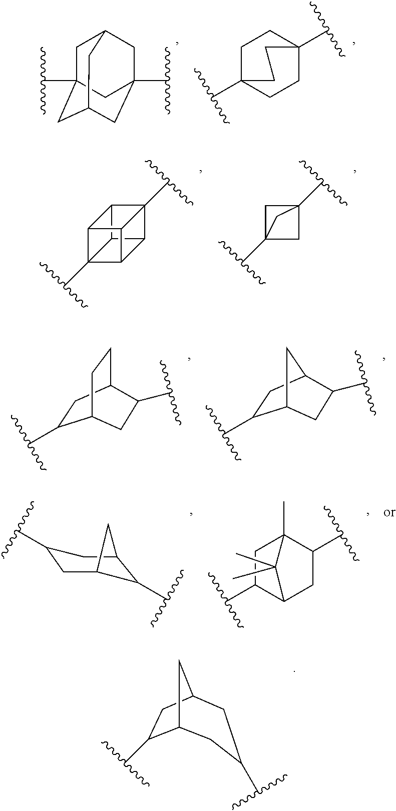

In another embodiment, Z² is selected from the group consisting of:

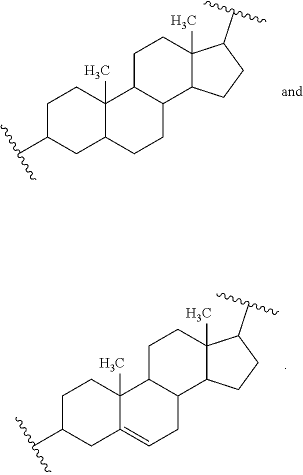 [SMILES: *C1CCC2(C)C(CCC3C4CCC(*)C4(C)CCC32)C1 ; *C1CCC2(C)C(=CCC3C2CCC2(C)C(*)CCC32)C1]

In another embodiment, the disclosure provides a compound selected from any one of more of the compounds of Table (III), or a pharmaceutically acceptable salt or solvate thereof.

TABLE (III)
Non-Limiting Examples of Ionizable Lipids with a Constrained Arm

| Compound | |
|---|---|
| No. | Structure |
| C1 | 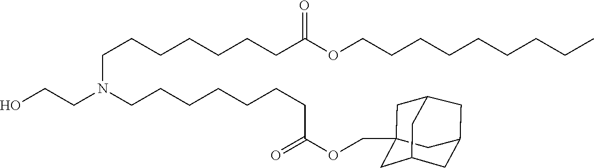 [SMILES: CCCCCCCCCOC(=O)CCCCCCCN(CCO)CCCCCCCC(=O)OCC12CC3CC(CC(C3)C1)C2] |
| C2 | 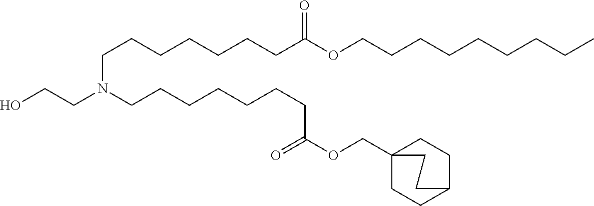 [SMILES: CCCCCCCCCOC(=O)CCCCCCCN(CCO)CCCCCCCC(=O)OCC12CCC(CC1)CC2] |
| C3 | 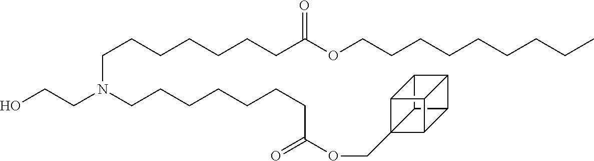 [SMILES: CCCCCCCCCOC(=O)CCCCCCCN(CCO)CCCCCCCC(=O)OCC12C3C4C5C3C1C5C42] |
| C4 | 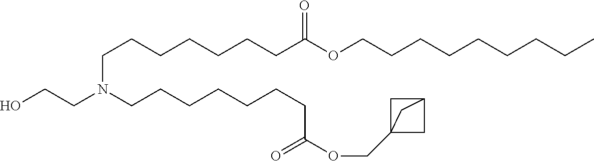 [SMILES: CCCCCCCCCOC(=O)CCCCCCCN(CCO)CCCCCCCC(=O)OCC12CC(C1)C2] |
| C5 | 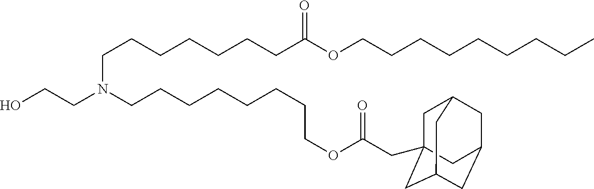 [SMILES: CCCCCCCCCOC(=O)CCCCCCCN(CCO)CCCCCCCCOC(=O)CC12CC3CC(CC(C3)C1)C2] |
| C6 | 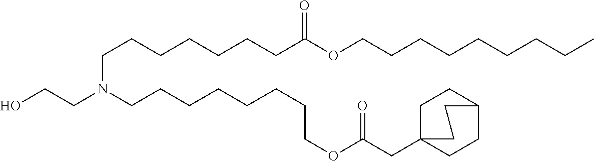 [SMILES: CCCCCCCCCOC(=O)CCCCCCCN(CCO)CCCCCCCCOC(=O)CC12CCC(CC1)CC2] |
| C7 | 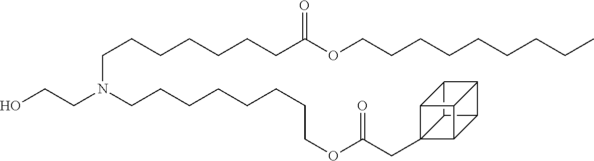 [SMILES: CCCCCCCCCOC(=O)CCCCCCCN(CCO)CCCCCCCCOC(=O)CC12C3C4C5C3C1C5C42] |
| C8 | 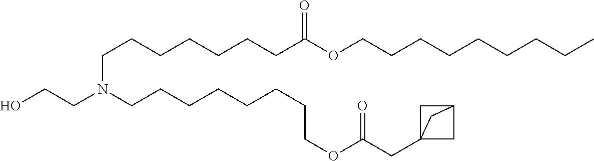 [SMILES: CCCCCCCCCOC(=O)CCCCCCCN(CCO)CCCCCCCCOC(=O)CC12CC(C1)C2] |
| C9 | 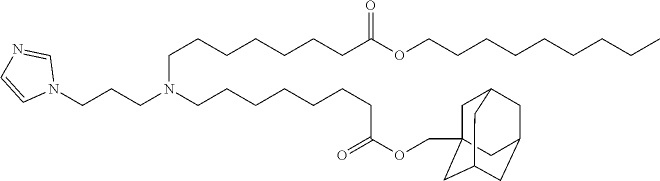 [SMILES: CCCCCCCCCOC(=O)CCCCCCCN(CCCCCCCC(=O)OCC12CC3CC(CC(C3)C1)C2)CCCn1ccnc1] |
| C10 | 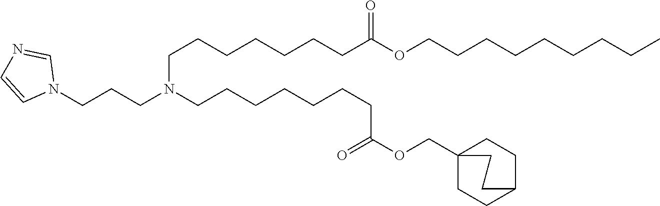 [SMILES: CCCCCCCCCOC(=O)CCCCCCCN(CCCCCCCC(=O)OCC12CCC(CC1)CC2)CCCn1ccnc1] |
| C11 | 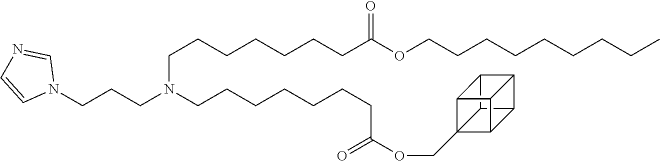 [SMILES: CCCCCCCCCOC(=O)CCCCCCCN(CCCCCCCC(=O)OCC12C3C4C5C3C1C5C42)CCCn1ccnc1] |
| C12 | 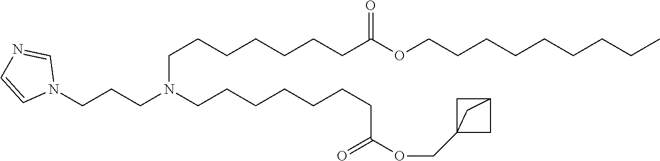 [SMILES: CCCCCCCCCOC(=O)CCCCCCCN(CCCCCCCC(=O)OCC12CC(C1)C2)CCCn1ccnc1] |
| C13 | 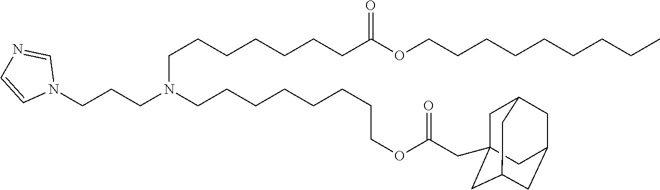 [SMILES: CCCCCCCCCOC(=O)CCCCCCCN(CCCCCCCCOC(=O)CC12CC3CC(CC(C3)C1)C2)CCCn1ccnc1] |
| C14 | 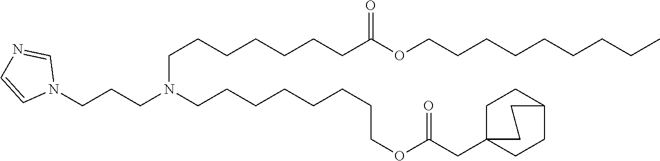 [SMILES: CCCCCCCCCOC(=O)CCCCCCCN(CCCCCCCCOC(=O)CC12CCC(CC1)CC2)CCCn1ccnc1] |
| C15 | 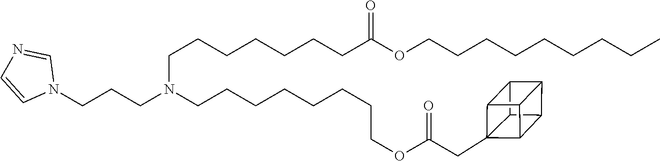 [SMILES: CCCCCCCCCOC(=O)CCCCCCCN(CCCCCCCCOC(=O)CC12C3C4C5C3C1C5C42)CCCn1ccnc1] |
| C16 | 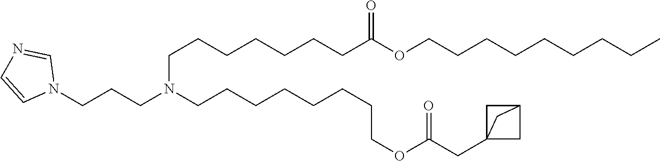 [SMILES: CCCCCCCCCOC(=O)CCCCCCCN(CCCCCCCCOC(=O)CC12CC(C1)C2)CCCn1ccnc1] |
| C17 | 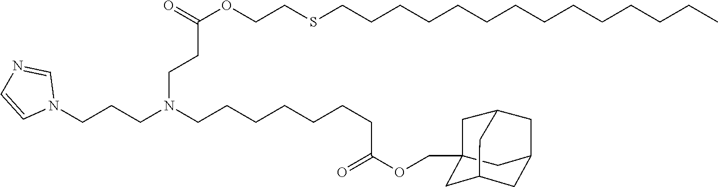 [SMILES: CCCCCCCCCCCCCCSCCOC(=O)CCN(CCCCCCCC(=O)OCC12CC3CC(CC(C3)C1)C2)CCCn1ccnc1] |
| C18 | 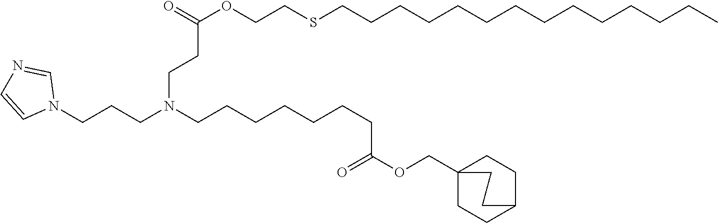 [SMILES: CCCCCCCCCCCCCCSCCOC(=O)CCN(CCCCCCCC(=O)OCC12CCC(CC1)CC2)CCCn1ccnc1] |
| C19 | 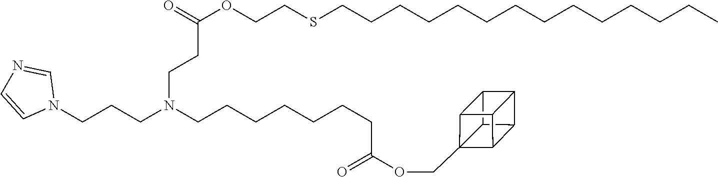 [SMILES: CCCCCCCCCCCCCCSCCOC(=O)CCN(CCCCCCCC(=O)OCC12C3C4C5C3C1C5C42)CCCn1ccnc1] |
| C20 | 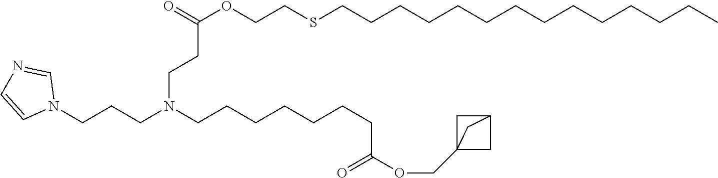 [SMILES: CCCCCCCCCCCCCCSCCOC(=O)CCN(CCCCCCCC(=O)OCC12CC(C1)C2)CCCn1ccnc1] |
| C21 | 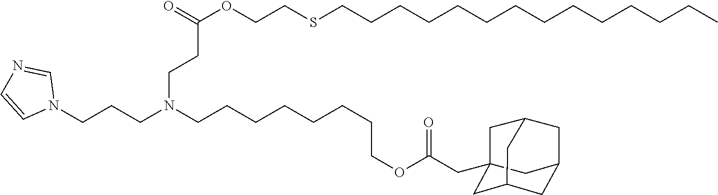 [SMILES: CCCCCCCCCCCCCCSCCOC(=O)CCN(CCCCCCCCOC(=O)CC12CC3CC(CC(C3)C1)C2)CCCn1ccnc1] |
| C22 | 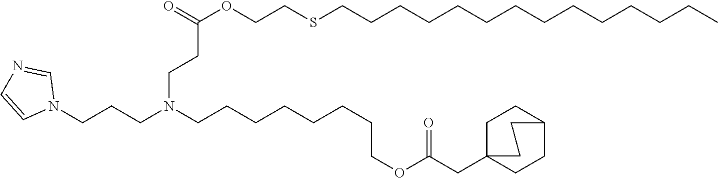 [SMILES: CCCCCCCCCCCCCCSCCOC(=O)CCN(CCCCCCCCOC(=O)CC12CCC(CC1)CC2)CCCn1ccnc1] |
| C23 | 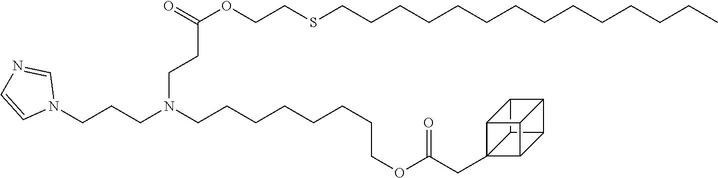 [SMILES: CCCCCCCCCCCCCCSCCOC(=O)CCN(CCCCCCCCOC(=O)CC12C3C4C5C3C1C5C42)CCCn1ccnc1] |
| C24 | 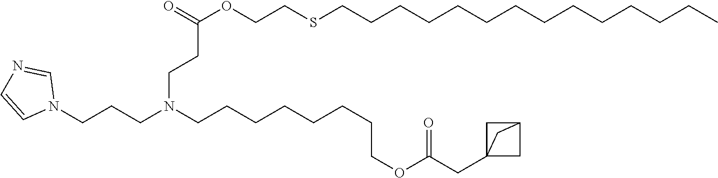 [SMILES: CCCCCCCCCCCCCCSCCOC(=O)CCN(CCCCCCCCOC(=O)CC12CC(C1)C2)CCCn1ccnc1] |
| C25 | 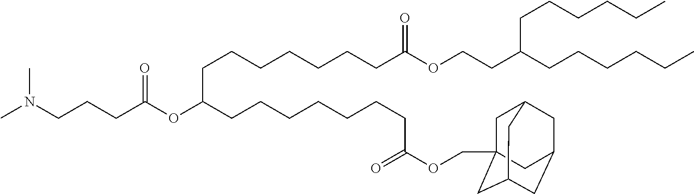 [SMILES: CCCCCCC(CCCCCC)CCOC(=O)CCCCCCCC(CCCCCCCC(=O)OCC12CC3CC(CC(C3)C1)C2)OC(=O)CCCN(C)C] |
| C26 | 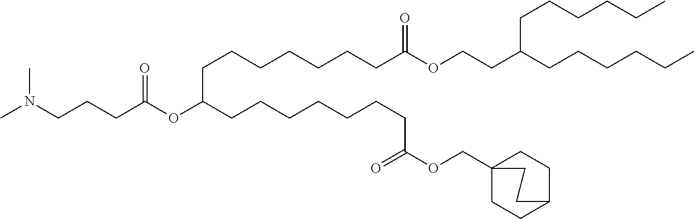 [SMILES: CCCCCCC(CCCCCC)CCOC(=O)CCCCCCCC(CCCCCCCC(=O)OCC12CCC(CC1)CC2)OC(=O)CCCN(C)C] |
| C27 | 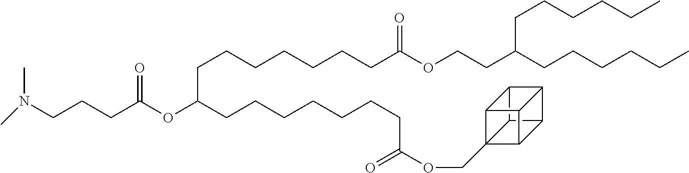 [SMILES: CCCCCCC(CCCCCC)CCOC(=O)CCCCCCCC(CCCCCCCC(=O)OCC12C3C4C5C3C1C5C42)OC(=O)CCCN(C)C] |
| | / |
| C28 | 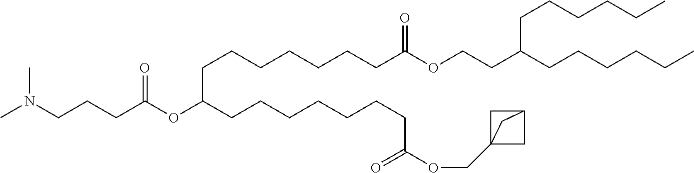 [SMILES: CCCCCCC(CCCCCC)CCOC(=O)CCCCCCCC(CCCCCCCC(=O)OCC12CC(C1)C2)OC(=O)CCCN(C)C] |
| C29 | 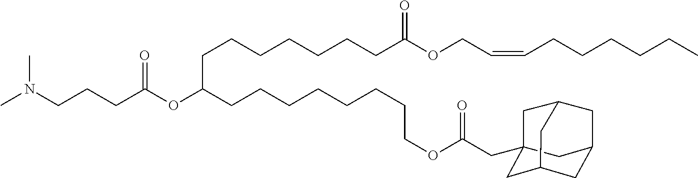 [SMILES: CCCCCC/C=C\COC(=O)CCCCCCCC(CCCCCCCCOC(=O)CC12CC3CC(CC(C3)C1)C2)OC(=O)CCCN(C)C] |
| C30 | 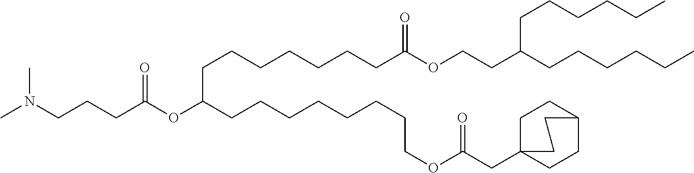 [SMILES: CCCCCCC(CCCCCC)CCOC(=O)CCCCCCCC(CCCCCCCCOC(=O)CC12CCC(CC1)CC2)OC(=O)CCCN(C)C] |
| C31 | 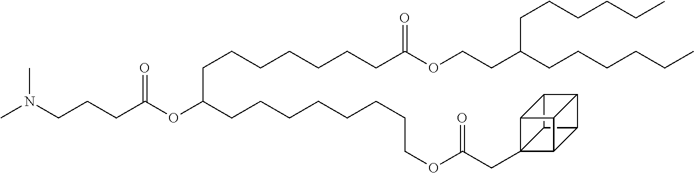 [SMILES: CCCCCCC(CCCCCC)CCOC(=O)CCCCCCCC(CCCCCCCCOC(=O)CC12C3C4C5C3C1C5C42)OC(=O)CCCN(C)C] |
| C32 | 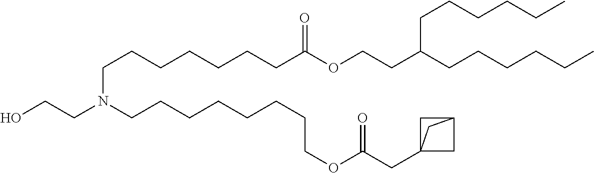 [SMILES: CCCCCCC(CCCCCC)CCOC(=O)CCCCCCCN(CCO)CCCCCCCCOC(=O)CC12CC(C1)C2] |
| C33 | 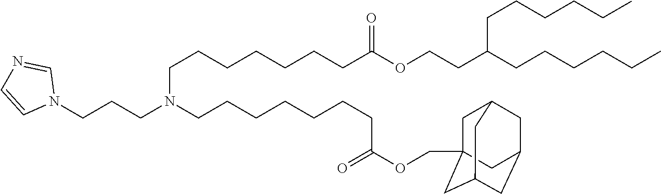 [SMILES: CCCCCCC(CCCCCC)CCOC(=O)CCCCCCCN(CCCCCCCC(=O)OCC12CC3CC(CC(C3)C1)C2)CCCn1ccnc1] |
| C34 | 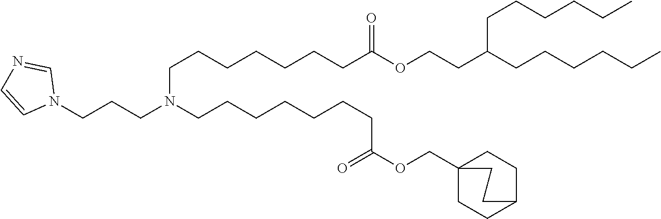 [SMILES: CCCCCCC(CCCCCC)CCOC(=O)CCCCCCCN(CCCCCCCC(=O)OCC12CCC(CC1)CC2)CCCn1ccnc1] |
| C35 | 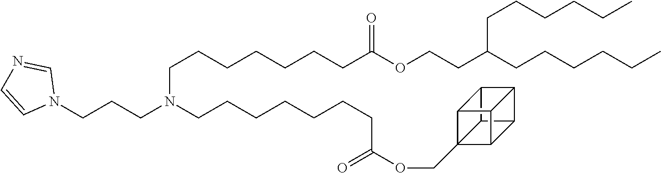 [SMILES: CCCCCCC(CCCCCC)CCOC(=O)CCCCCCCN(CCCCCCCC(=O)OCC12C3C4C5C3C1C5C42)CCCn1ccnc1] |
| C36 | 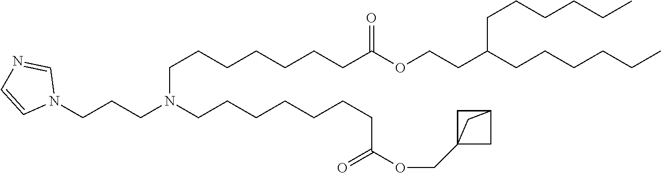 [SMILES: CCCCCCC(CCCCCC)CCOC(=O)CCCCCCCN(CCCCCCCC(=O)OCC12CC(C1)C2)CCCn1ccnc1] |
| C37 | 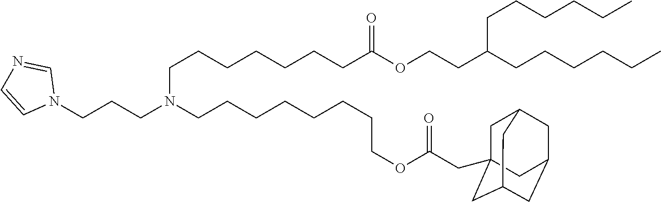 [SMILES: CCCCCCC(CCCCCC)CCOC(=O)CCCCCCCN(CCCCCCCCOC(=O)CC12CC3CC(CC(C3)C1)C2)CCCn1ccnc1] |
| C38 | 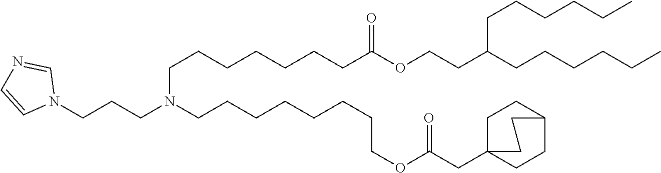 [SMILES: CCCCCCC(CCCCCC)CCOC(=O)CCCCCCCN(CCCCCCCCOC(=O)CC12CCC(CC1)CC2)CCCn1ccnc1] |
| C39 | 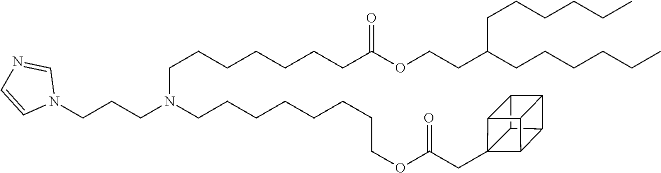 [SMILES: CCCCCCC(CCCCCC)CCOC(=O)CCCCCCCN(CCCCCCCCOC(=O)CC12C3C4C5C3C1C5C42)CCCn1ccnc1] |
| C40 | 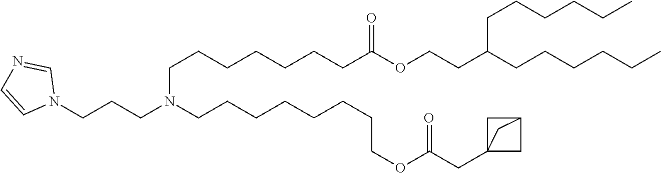 [SMILES: CCCCCCC(CCCCCC)CCOC(=O)CCCCCCCN(CCCCCCCCOC(=O)CC12CC(C1)C2)CCCn1ccnc1] |
| C41 | 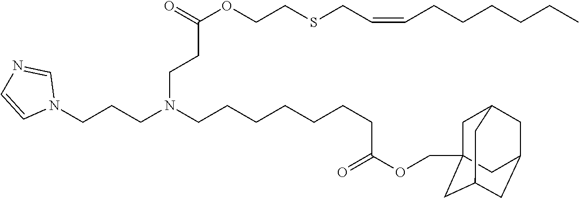 [SMILES: CCCCCC/C=C\CSCCOC(=O)CCN(CCCCCCCC(=O)OCC12CC3CC(CC(C3)C1)C2)CCCn1ccnc1] |
| C42 | 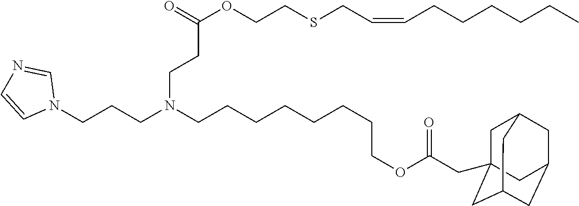 [SMILES: CCCCCC/C=C\CSCCOC(=O)CCN(CCCCCCCCOC(=O)CC12CC3CC(CC(C3)C1)C2)CCCn1ccnc1] |
| C43 | 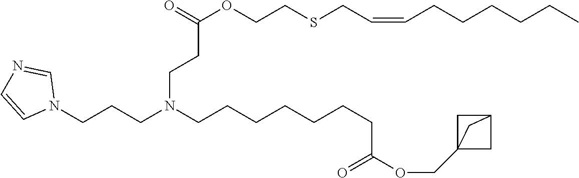 [SMILES: CCCCCC/C=C\CSCCOC(=O)CCN(CCCCCCCC(=O)OCC12CC(C1)C2)CCCn1ccnc1] |
| C44 | 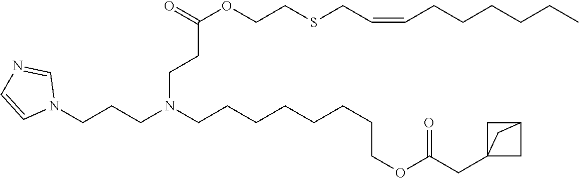 [SMILES: CCCCCC/C=C\CSCCOC(=O)CCN(CCCCCCCCOC(=O)CC12CC(C1)C2)CCCn1ccnc1] |
| C45 | 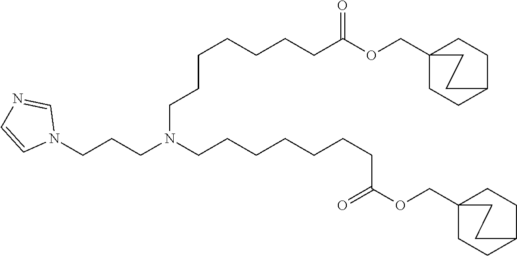 [SMILES: O=C(CCCCCCCN(CCCCCCCC(=O)OCC12CCC(CC1)CC2)CCCn1ccnc1)OCC12CCC(CC1)CC2] |
| C46 | 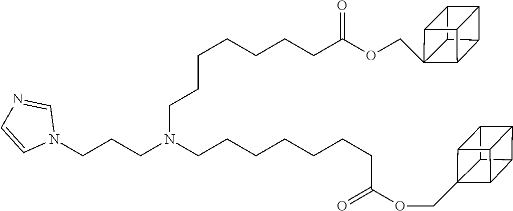 [SMILES: O=C(CCCCCCCN(CCCCCCCC(=O)OCC12C3C4C5C3C1C5C42)CCCn1ccnc1)OCC12C3C4C5C3C1C5C42] |
| C47 | 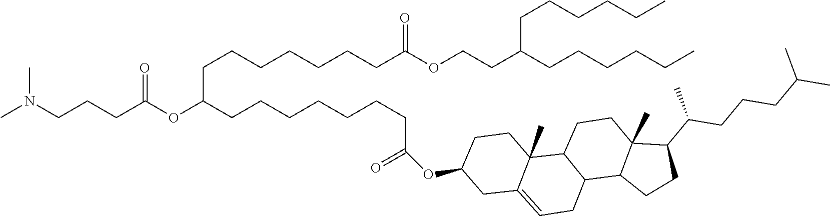 |
| C48 | 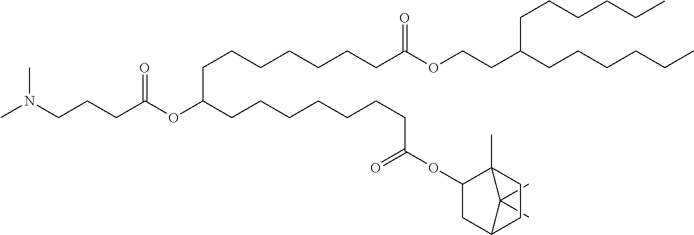 [SMILES: CCCCCCC(CCCCCC)CCOC(=O)CCCCCCCC(CCCCCCCC(=O)OC1CC2CCC1(C)C2(C)C)OC(=O)CCCN(C)C] |
| C49 | 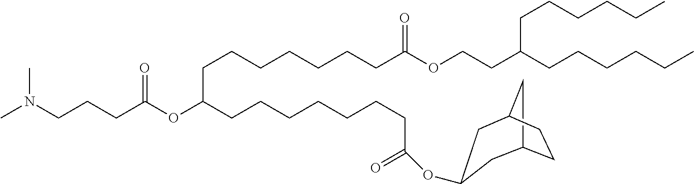 [SMILES: CCCCCCC(CCCCCC)CCOC(=O)CCCCCCCC(CCCCCCCC(=O)OC1CC2CCC(C2)C1)OC(=O)CCCN(C)C] |
| C50 | 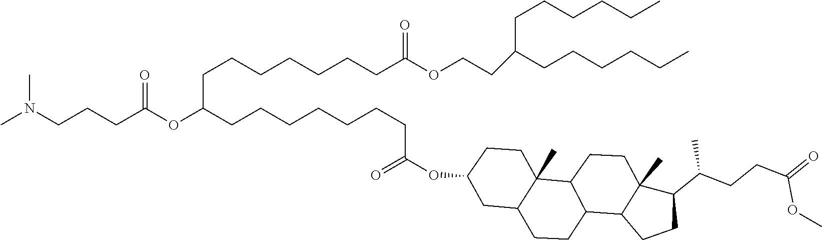 |
| C51 | 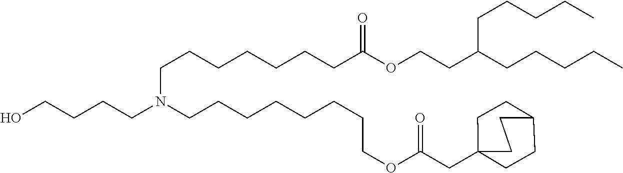 [SMILES: CCCCCC(CCCCC)CCOC(=O)CCCCCCCN(CCCCO)CCCCCCCCOC(=O)CC12CCC(CC1)CC2] |
| C52 | 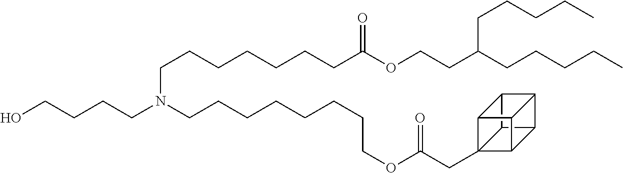 [SMILES: CCCCCC(CCCCC)CCOC(=O)CCCCCCCN(CCCCO)CCCCCCCCOC(=O)CC12C3C4C5C3C1C5C42] |
| C53 | 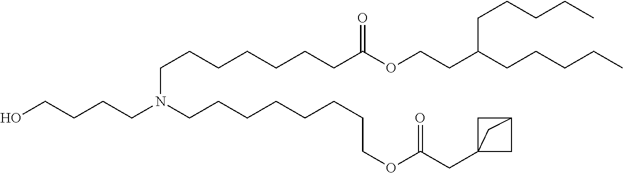 [SMILES: CCCCCC(CCCCC)CCOC(=O)CCCCCCCN(CCCCO)CCCCCCCCOC(=O)CC12CC(C1)C2] |
| C54 | 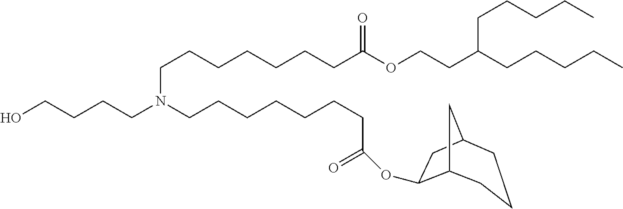 [SMILES: CCCCCC(CCCCC)CCOC(=O)CCCCCCCN(CCCCO)CCCCCCCC(=O)OC1CC2CCCC1C2] |
| C55 | 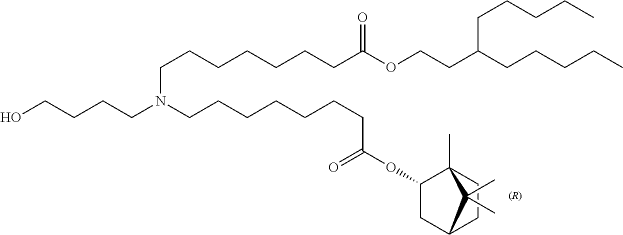 |
| C56 | 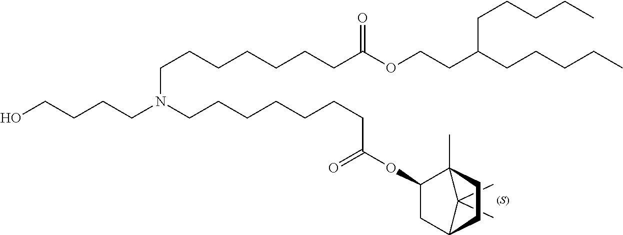 |
| C57 | 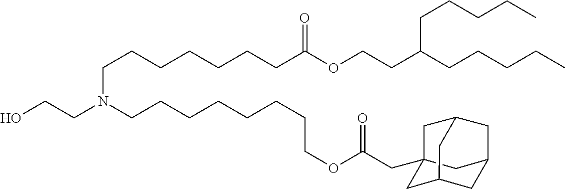 [SMILES: CCCCCC(CCCCC)CCOC(=O)CCCCCCCN(CCO)CCCCCCCCOC(=O)CC12CC3CC(CC(C3)C1)C2] |
| C58 | 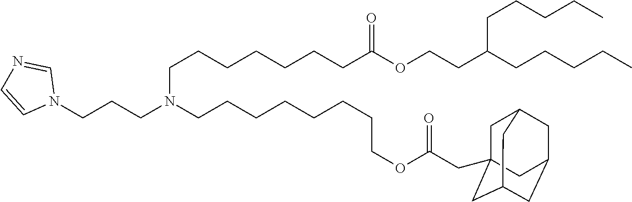 [SMILES: CCCCCC(CCCCC)CCOC(=O)CCCCCCCN(CCCCCCCCOC(=O)CC12CC3CC(CC(C3)C1)C2)CCCn1ccnc1] |
| C59 | 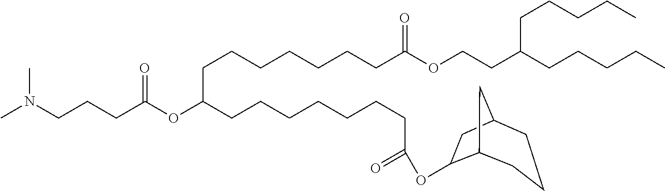 [SMILES: CCCCCC(CCCCC)CCOC(=O)CCCCCCCC(CCCCCCCC(=O)OC1CC2CCCC1C2)OC(=O)CCCN(C)C] |
| C60 | 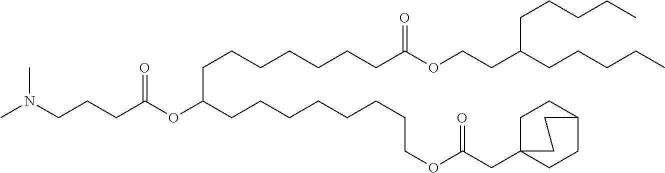 [SMILES: CCCCCC(CCCCC)CCOC(=O)CCCCCCCC(CCCCCCCCOC(=O)CC12CCC(CC1)CC2)OC(=O)CCCN(C)C] |
| C61 | 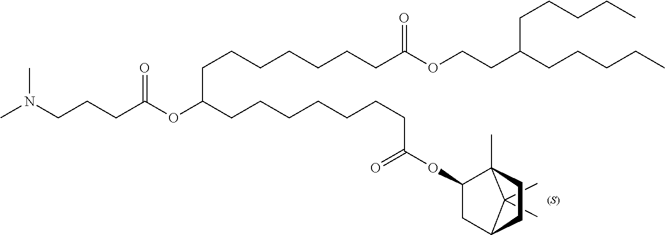 |
| C62 | 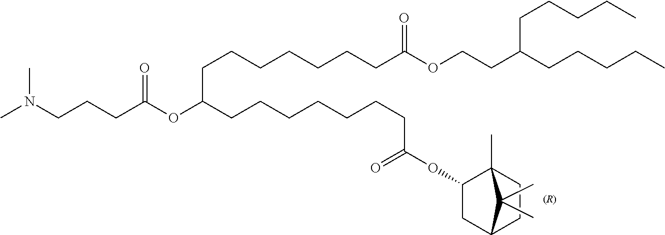 |
| C63 | 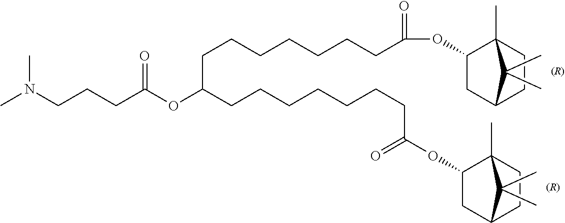 |
| C64 | 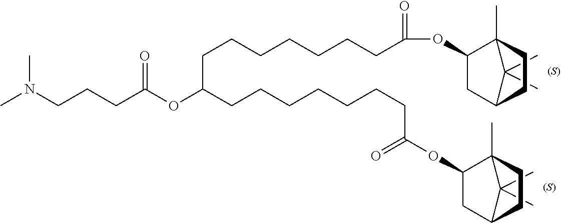 |
| C65 | 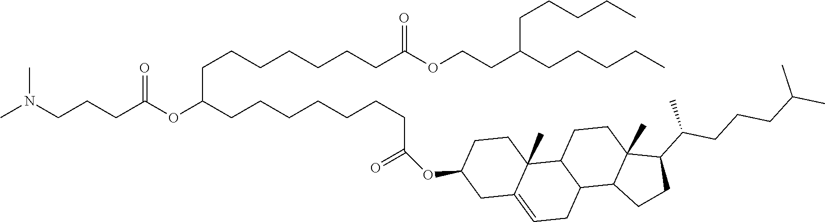 |
| C66 | 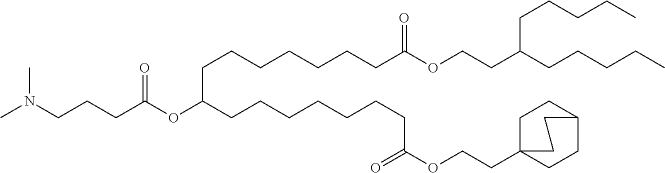 [SMILES: CCCCCC(CCCCC)CCOC(=O)CCCCCCCC(CCCCCCCC(=O)OCCC12CCC(CC1)CC2)OC(=O)CCCN(C)C] |
| C67 | 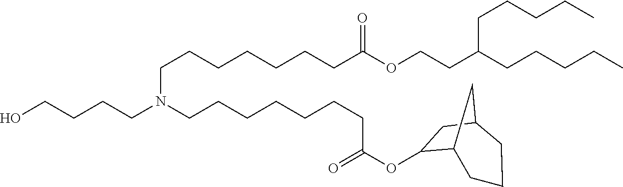 [SMILES: CCCCCC(CCCCC)CCOC(=O)CCCCCCCN(CCCCO)CCCCCCCC(=O)OC1CC2CCCC1C2] |
| C68 | 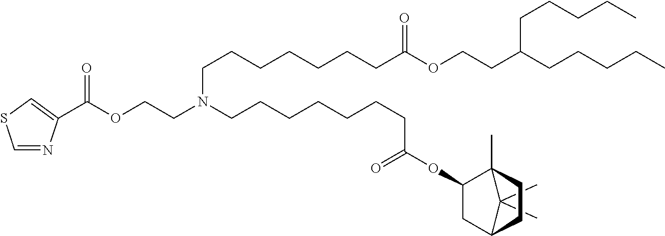 |
| C69 | 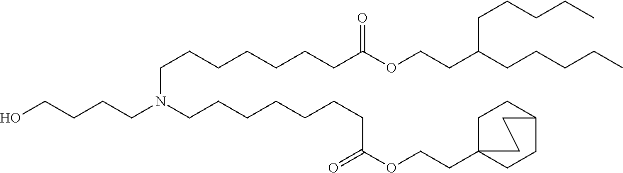 [SMILES: CCCCCC(CCCCC)CCOC(=O)CCCCCCCN(CCCCO)CCCCCCCC(=O)OCCC12CCC(CC1)CC2] |
| C70 | 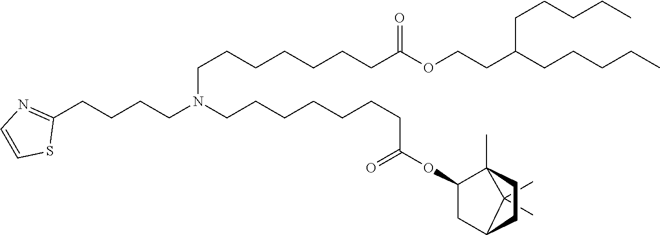 |
| C71 | 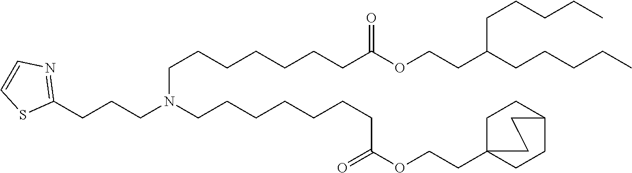 [SMILES: CCCCCC(CCCCC)CCOC(=O)CCCCCCCN(CCCCCCCC(=O)OCCC12CCC(CC1)CC2)CCCc1nccs1] |
| C72 | 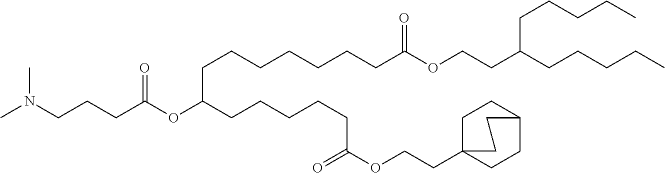 [SMILES: CCCCCC(CCCCC)CCOC(=O)CCCCCCCC(CCCCCC(=O)OCCC12CCC(CC1)CC2)OC(=O)CCCN(C)C] |
| C73 | 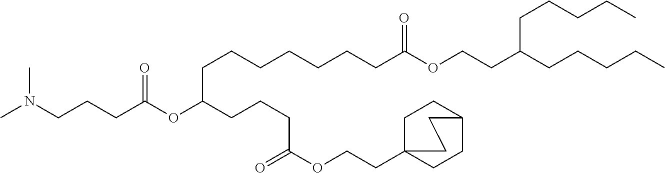 [SMILES: CCCCCC(CCCCC)CCOC(=O)CCCCCCCC(CCCC(=O)OCCC12CCC(CC1)CC2)OC(=O)CCCN(C)C] |
| C74 | 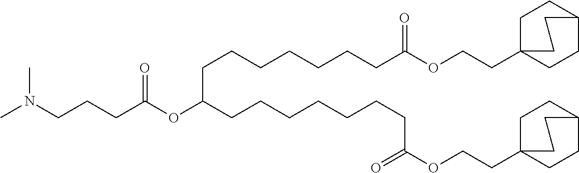 [SMILES: CN(C)CCCC(=O)OC(CCCCCCCC(=O)OCCC12CCC(CC1)CC2)CCCCCCCC(=O)OCCC12CCC(CC1)CC2] |
| C75 | 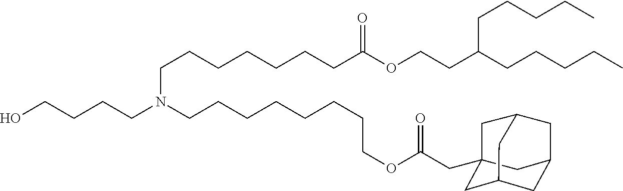 [SMILES: CCCCCC(CCCCC)CCOC(=O)CCCCCCCN(CCCCO)CCCCCCCCOC(=O)CC12CC3CC(CC(C3)C1)C2] |
| C76 | 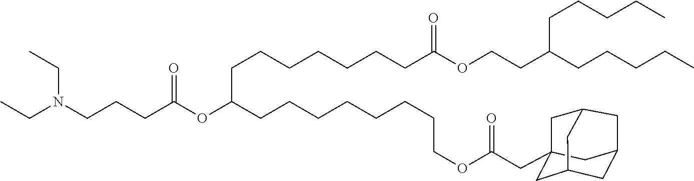 [SMILES: CCCCCC(CCCCC)CCOC(=O)CCCCCCCC(CCCCCCCCOC(=O)CC12CC3CC(CC(C3)C1)C2)OC(=O)CCCN(CC)CC] |
| C77 | 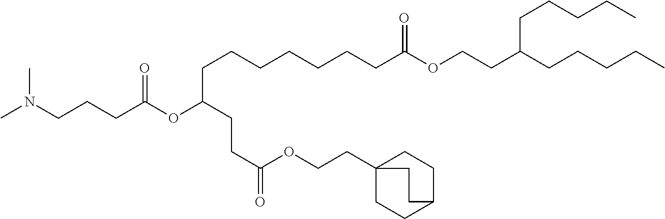 [SMILES: CCCCCC(CCCCC)CCOC(=O)CCCCCCCC(CCC(=O)OCCC12CCC(CC1)CC2)OC(=O)CCCN(C)C] |
| C78 | 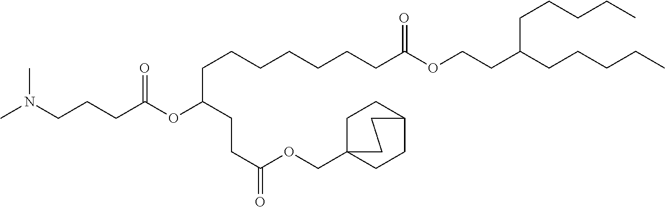 [SMILES: CCCCCC(CCCCC)CCOC(=O)CCCCCCCC(CCC(=O)OCC12CCC(CC1)CC2)OC(=O)CCCN(C)C] |
| C79 | 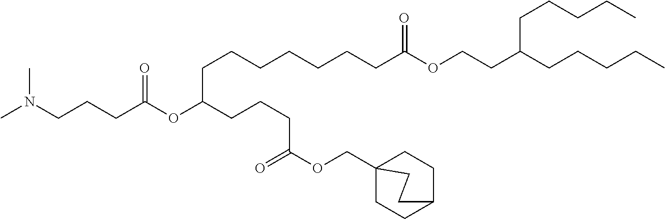 [SMILES: CCCCCC(CCCCC)CCOC(=O)CCCCCCCC(CCCC(=O)OCC12CCC(CC1)CC2)OC(=O)CCCN(C)C] |
| C80 | 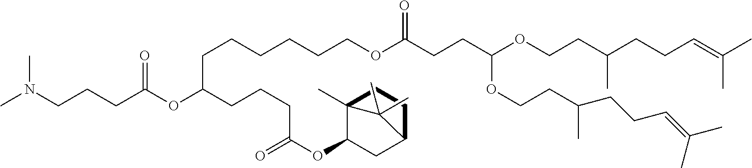 |
| C81 | 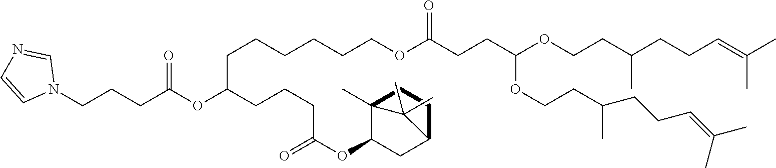 |

In some embodiments, an LNP of the present disclosure comprises an ionizable lipid disclosed in PCT Application PCT/US2023/065477, which is incorporated by reference herein, in its entirety.

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, a compound of the present disclosure is represented by Formula (CX-I):

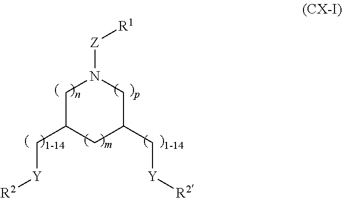

or a pharmaceutically acceptable salt thereof, wherein

Z is selected from the group consisting of a bond,

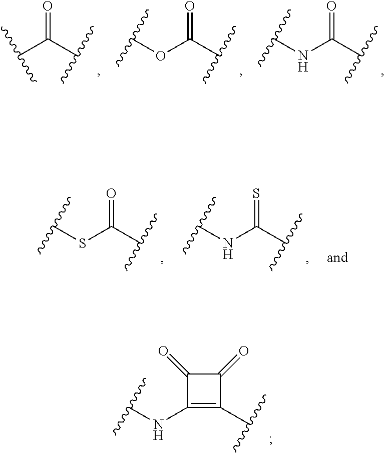

each Y is independently selected from the group consisting of

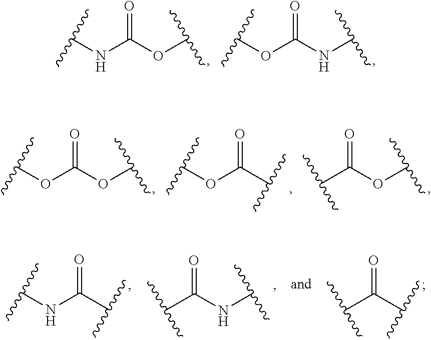

R¹ is —(CH₂)₁₋₆N(R^{a})₂ or —(CH₂)₁₋₆OH;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
R^{2′} is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—; each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-i):

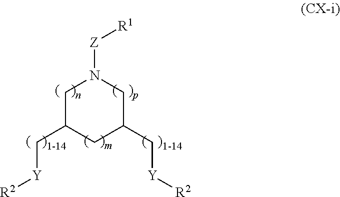

or a pharmaceutically acceptable salt thereof,
wherein
Z is selected from the group consisting of a bond,

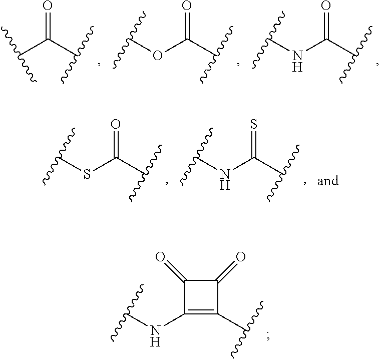

each Y is independently selected from the group consisting of

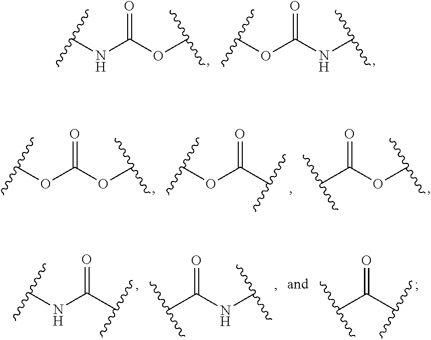

R¹ is —(CH₂)₁₋₆N(R^{a})₂;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
in is 0, 1, or 2;
n is 1 or 2; and
p is 1 or 2.

In some embodiments, a compound of the present disclosure is represented by Formula (CX-I′), (CX-I″), (CX-I′″),

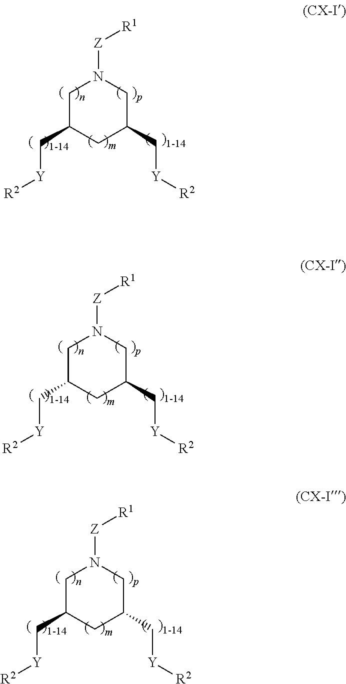

or a pharmaceutically acceptable salt thereof.

In some embodiments, a compound of the present disclosure is represented by Formula (CX-I-a), (CX-I-b), (CX-I-c), or (CX-I-d):

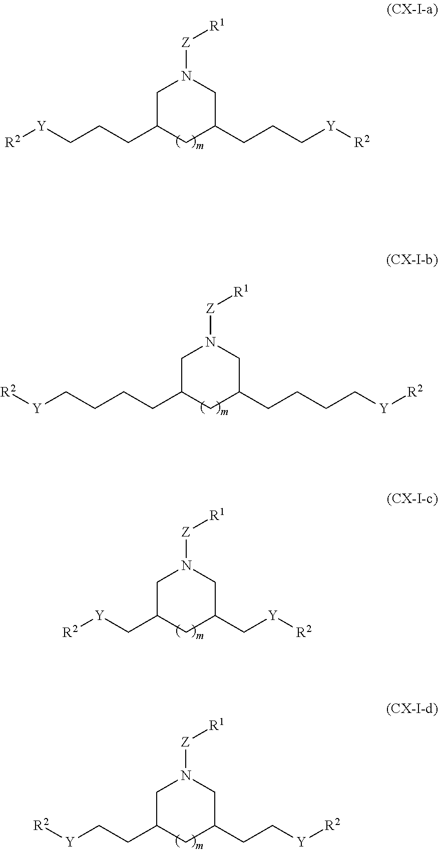

or a pharmaceutically acceptable salt thereof.

In some embodiments, a compound of the present disclosure is represented by Formula (CX-I-a′), (CX-I-b′), (CX-I-c′), or (CX-I-d′):

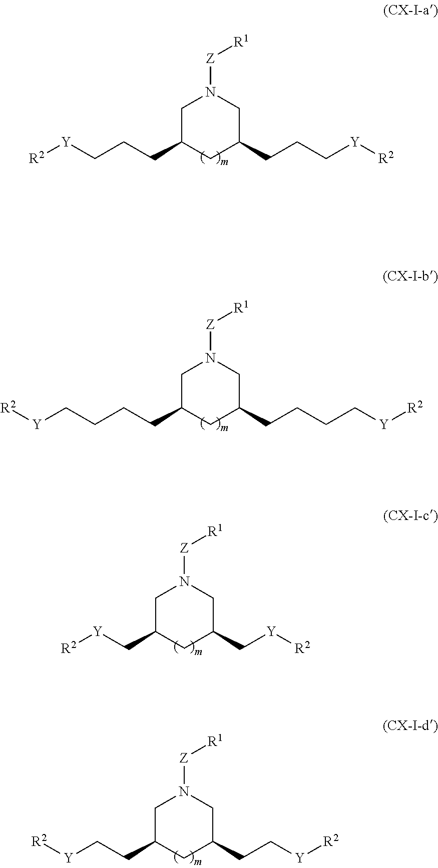

or a pharmaceutically acceptable salt thereof.

In some embodiments, a compound of the present disclosure is represented by Formula (CX-I-a″), (CX-I-b″), (CX-I-c″), or (CX-I-d″):

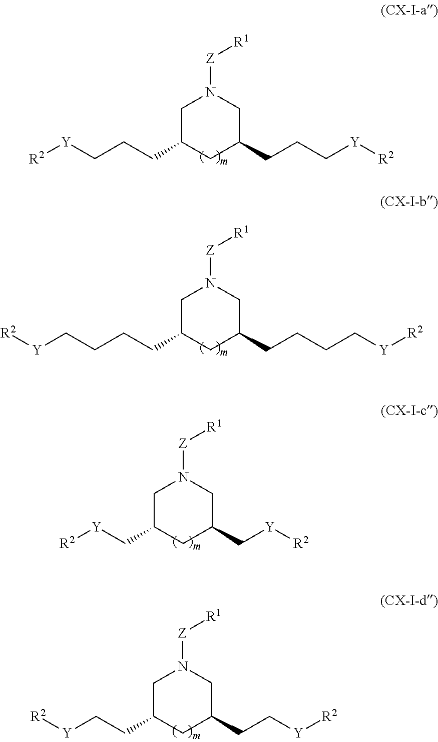

or a pharmaceutically acceptable salt thereof.

In some embodiments, a compound of the present disclosure is represented by Formula (CX-1-a′″), (CX-1-b′″), (CX-1-c′″), or (CX-1-d′″):

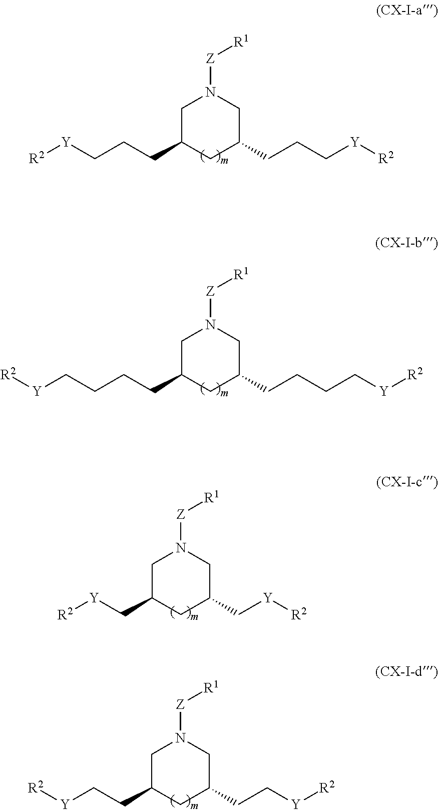

or a pharmaceutically acceptable salt thereof.

In some embodiments, a compound of the present disclosure is represented by Formula (CX-I-e) or (CX-I-f):

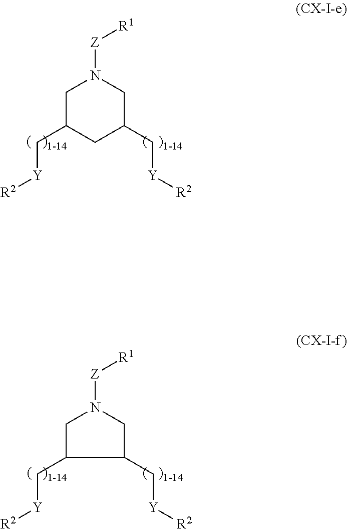

or a pharmaceutically acceptable salt thereof.

In some embodiments, a compound of the present disclosure is represented by Formula (CX-I-e′) or (CX-I-f′):

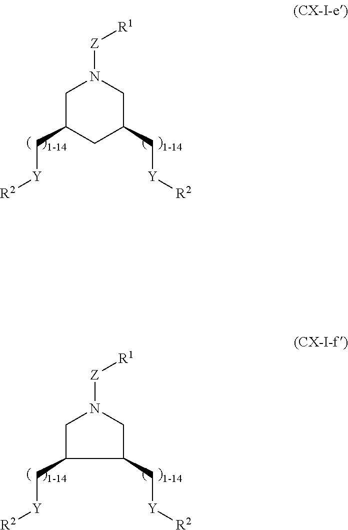

or a pharmaceutically acceptable salt thereof.

In some embodiments, a compound of the present disclosure is represented by Formula (CX-I-e″) or (CX-I-f″):

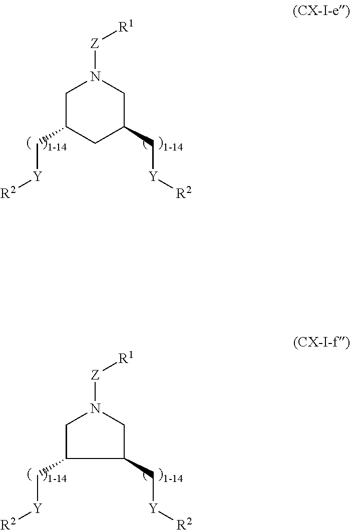

or a pharmaceutically acceptable salt thereof.

In some embodiments, a compound of the present disclosure is represented by Formula (CX-I-e′″) or (CX-I-f′″):

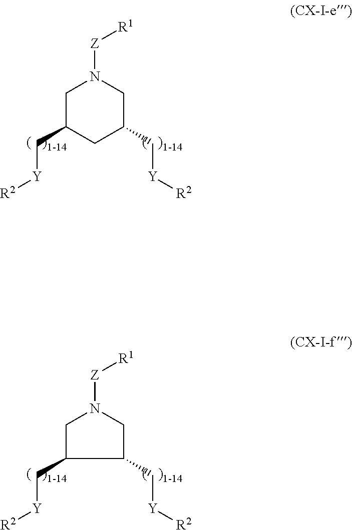

or a pharmaceutically acceptable salt thereof.

In some embodiments, a compound of the present disclosure is represented by Formula (CX-II):

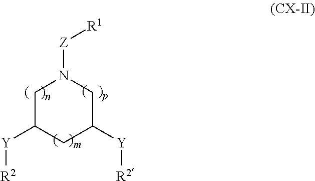

or a pharmaceutically acceptable salt thereof,
wherein
Z is selected from the group consisting of a bond,

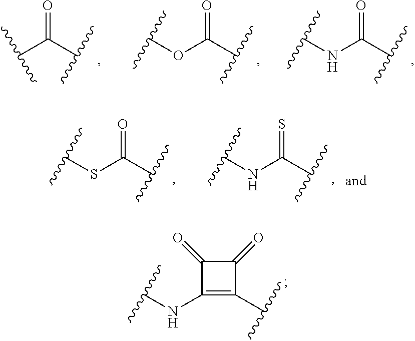

each Y is independently selected from the group consisting of

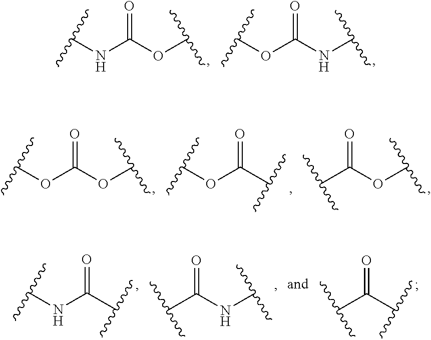

R¹ is —(CH₂)₁₋₆N(R^{a})₂ or —(CH₂)₁₋₆OH;
R² is optionally substituted C₅-C₃₆ alkyl or optionally substituted C₅-C₃₆ alkenyl, wherein 2 methylene units of R² are replaced with —O— to form an acetal within R², and wherein 1-6 methylene units of R² are optionally replaced with a group each independently selected from cyclopropylene, —O—, —OC(O)—, and —C(O)O—;
R^{2′} is optionally substituted C₁-C₃₆ alkyl or optionally substituted C₅-C₃₆ alkenyl, wherein 1-6 methylene units of R^{2′} are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CX-ii):

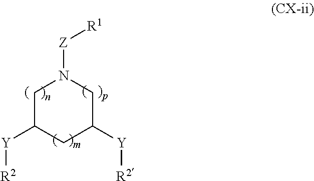

or a pharmaceutically acceptable salt thereof,
wherein
Z is selected from the group consisting of a bond,

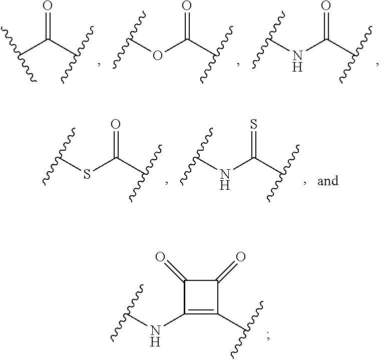

each Y is independently selected from the group consisting of

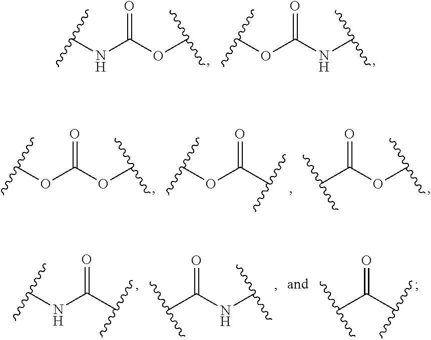

R¹ is —(CH₂)₁₋₆N(R^{a})₂;
R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
R^{2′} is optionally substituted C₁-C₃₆ alkyl, wherein 1-6 methylene units of R^{2′} are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by

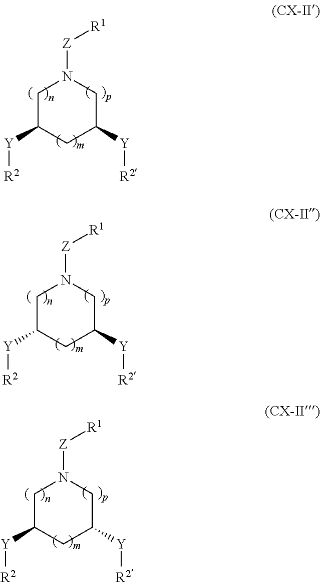

or a pharmaceutically acceptable salt thereof.

In some embodiments, a compound of the present disclosure is represented by Formula (CX-II-a)

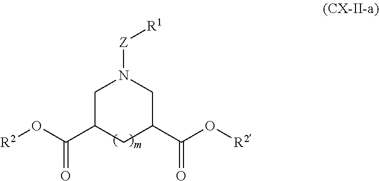

or a pharmaceutically acceptable salt thereof.

In some embodiments, a compound of the present disclosure is represented by Formula (CX-II-a′), (CX-II-a″), or (CX-II-a′″),

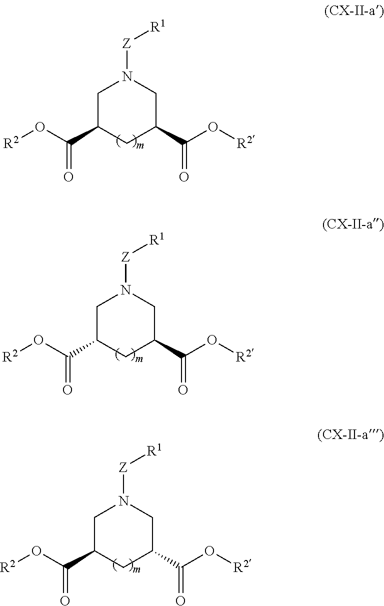

or a pharmaceutically acceptable salt thereof.

In some embodiments, a compound of the present disclosure is represented by Formula (CX-II-b), (CX-II-c), or (CX-II-d)

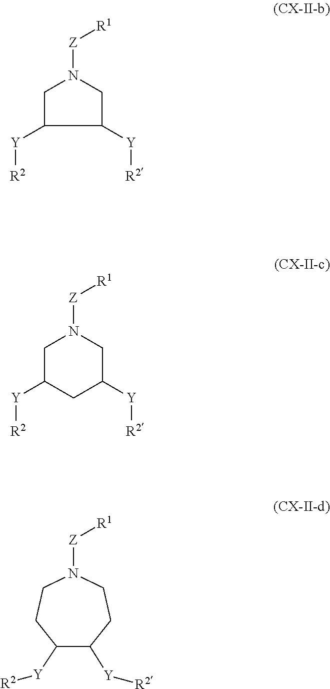

or a pharmaceutically acceptable salt thereof.

In some embodiments, a compound of the present disclosure is represented by Formula (CX-II-b′), (CX-II-c′), or (CX-II-d′)

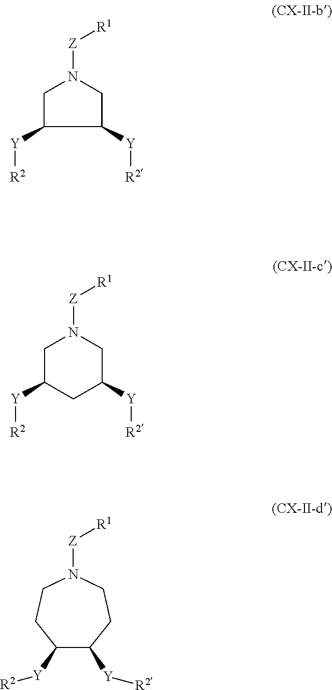

or a pharmaceutically acceptable salt thereof.

In some embodiments, a compound of the present disclosure is represented by Formula (CX-II-b″), (CX-II-c″), or (CX-II-d″)

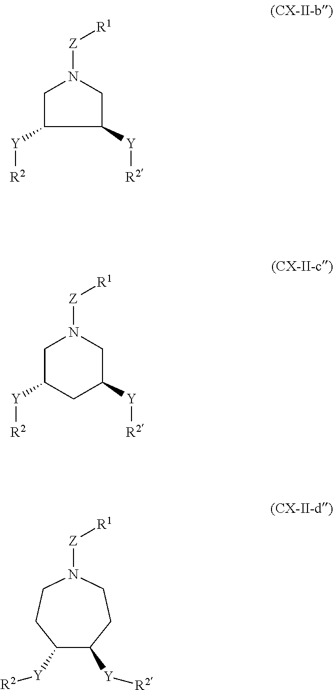

or a pharmaceutically acceptable salt thereof.

In some embodiments, a compound of the present disclosure is represented by Formula (CX-II-b′″), (CX-II-c′″), or (CX-II-d′″)

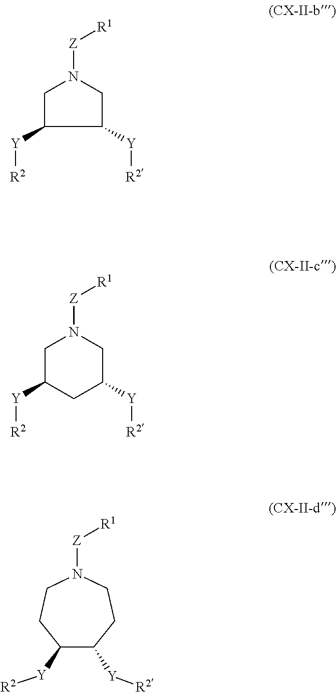

or a pharmaceutically acceptable salt thereof.

In some embodiments, a compound of the present disclosure is represented by is represented by formula (CX-II-e):

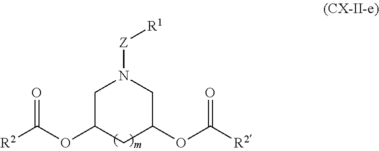

or a pharmaceutically acceptable salt thereof.

In some embodiments, a compound of the present disclosure is represented by Formula (CX-III)

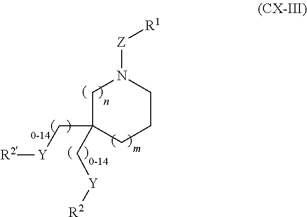

or a pharmaceutically acceptable salt thereof,
wherein
Z is selected from the group consisting of a bond,

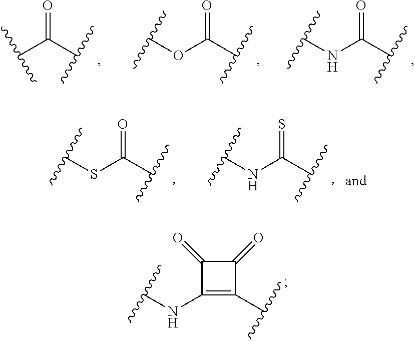

each Y is independently selected from the group consisting of

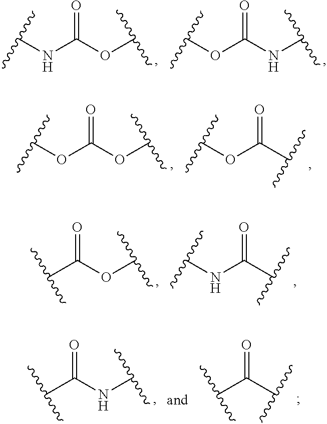

R¹ is —(CH₂)₁₋₆N(R^{a})₂ or —(CH₂)₁₋₆OH;
R² is optionally substituted C₅-C₃₆ alkyl or optionally substituted C₅-C₃₆ alkenyl, wherein 2 methylene units of R² are replaced with —O— to form an acetal within R², and wherein 1-6 methylene units of R² are optionally replaced with a group each independently selected from cyclopropylene, —O—, —OC(O)—, and —C(O)O—;
R^{2′} is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2; and
n is 1 or 2.

In some embodiments, a compound of the present disclosure is represented by Formula (CX-iii)

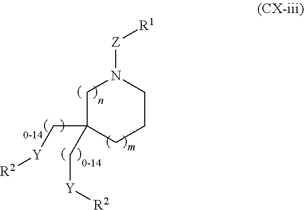

or a pharmaceutically acceptable salt thereof,
wherein
Z is selected from the group consisting of a bond,

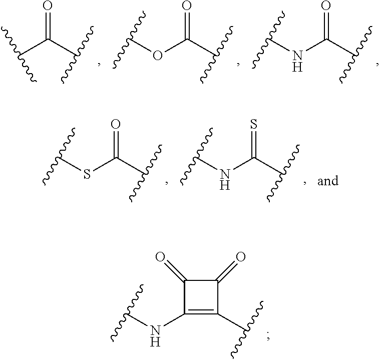

each Y is independently selected from the group consisting of

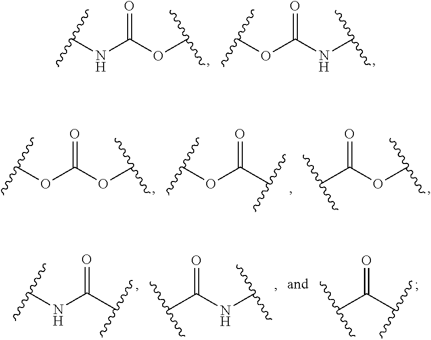

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2; and
n is 1 or 2.

In some embodiments, a compound of the present disclosure is represented by Formula (CX-III-a), (CX-III-b), or (CX-III-c):

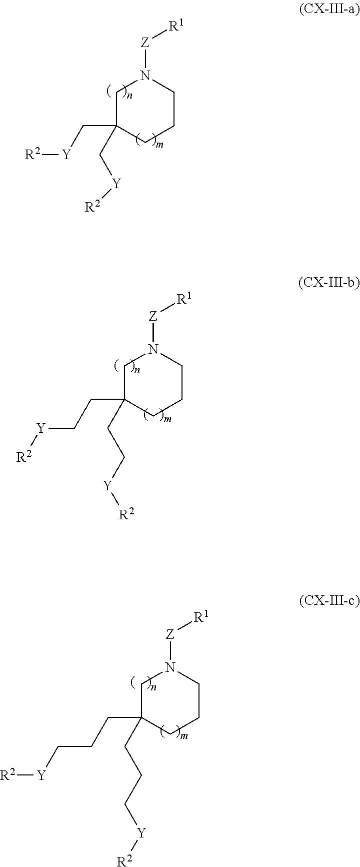

or a pharmaceutically acceptable salt thereof.

In some embodiments, a compound of the present disclosure is represented by Formula (CX-III-d) or (CX-III-e)

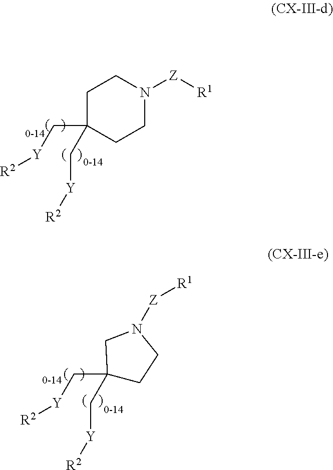

or a pharmaceutically acceptable salt thereof.

In some embodiments, a compound of the present disclosure is represented by Formula (CX-IV)

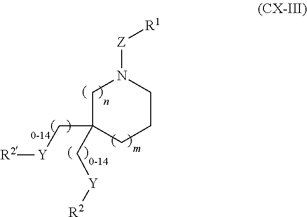

or a pharmaceutically acceptable salt thereof,
wherein
Z is selected from the group consisting of a bond,

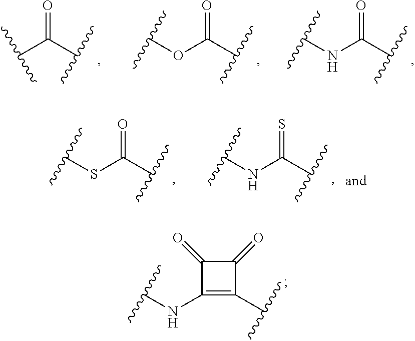

each Y is independently selected from the group consisting of

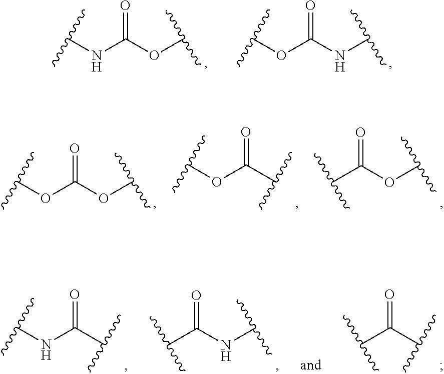

R¹ is —(CH₂)₁₋₆N(R^{a})₂ or —(CH₂)₁₋₆OH;
R² is C₃-C₃₆ branched alkyl or optionally substituted C₃-C₃₆ branched alkenyl, wherein 1-6 methylene units of R² are optionally replaced with a group each independently selected from cyclopropylene and —O—;
R^{2′} is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2; and
n is 1 or 2.

In some embodiments, a compound is represented by formula (CX-IV-a), (CX-IV-b), or (CX-IV-c):

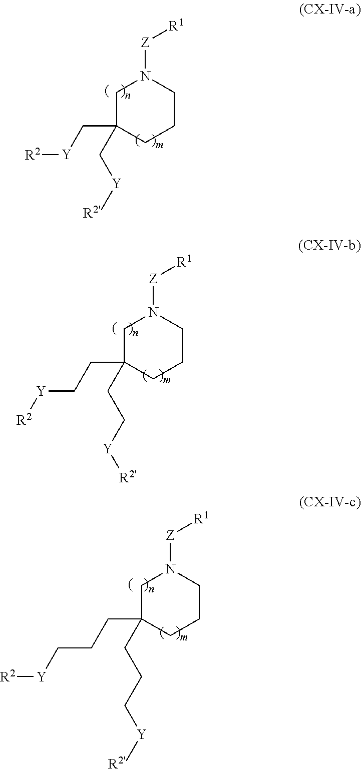

or a pharmaceutically acceptable salt thereof.

In some embodiments, a compound of the present disclosure compound is represented by formula (CX-IV-d) or (CX-IV-e):

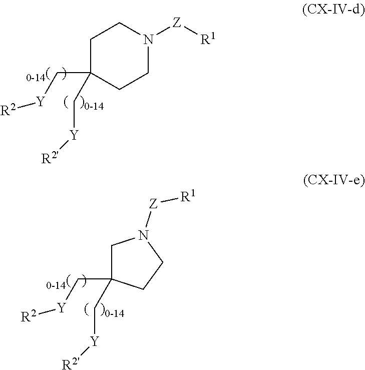

or a pharmaceutically acceptable salt thereof.

### Z

In some embodiments, Z is selected from the group consisting of a bond,

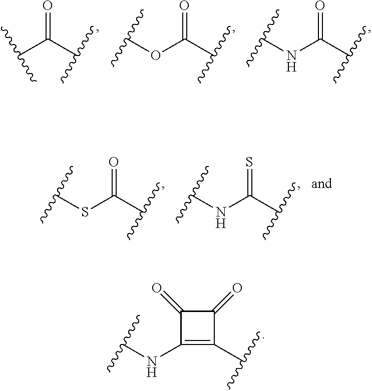

In some embodiments, Z is selected from the group consisting of,

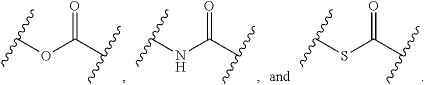 [SMILES: *OC(*)=O ; *NC(*)=O ; *SC(*)=O]

In some embodiments, Z is

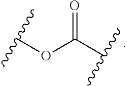 [SMILES: *OC(*)=O]

In some embodiments, Z is selected from the group consisting of a bond,

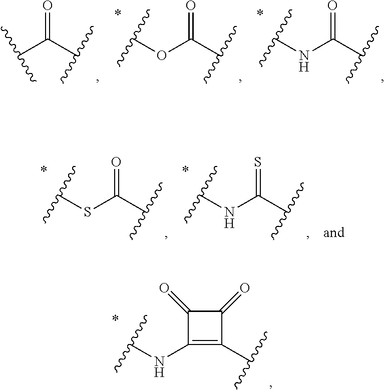

wherein R¹ is attached at the position denoted by *.

In some embodiments, Z is selected from the group consisting of

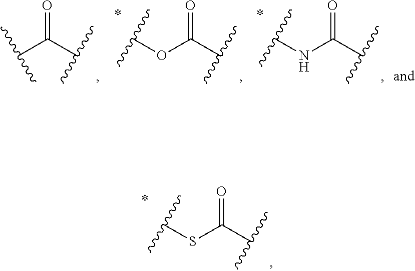

wherein R¹ is attached at the position denoted by *.

In some embodiments, Z is

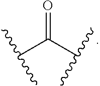 [SMILES: *C(*)=O]

In some embodiments, Z is

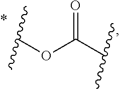

wherein R¹ is attached at the position denoted by *. In some embodiments, Z is

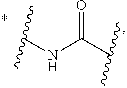

wherein R¹ is attached at the position denoted by *. In some embodiments, Z is

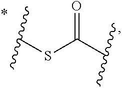

wherein R¹ is attached at the position denoted by *.

### Y

In some embodiments, each Y is independently selected from the group consisting

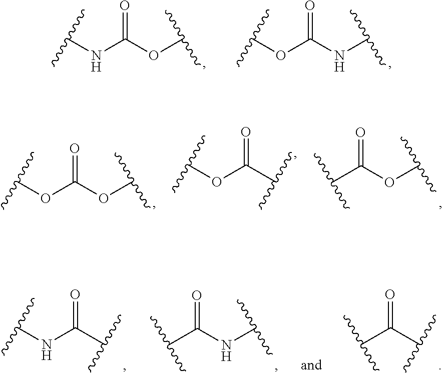

In some embodiments, Y is selected from the group consisting of

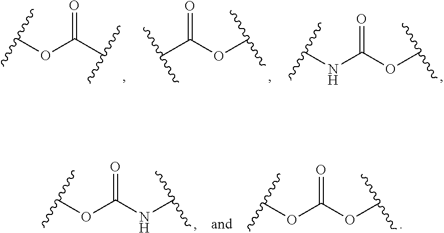 [SMILES: *OC(*)=O ; *OC(*)=O ; *NC(=O)O* ; *NC(=O)O* ; *OC(=O)O*]

In some embodiments, Y is selected from the group consisting of

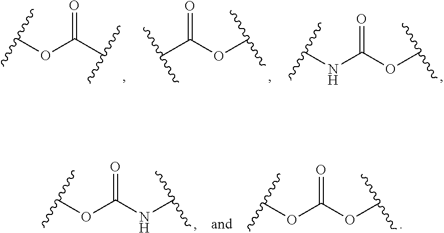

wherein R² is attached at the position denoted by *.

In some embodiments, Y is

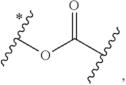

wherein R² is attached at the position denoted by *.

In some embodiments, Y is

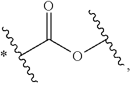

wherein R² is attached at the position denoted by *.

In some embodiments, Y is

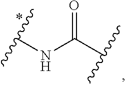

wherein R² is attached at the position denoted by *.

In some embodiments, Y is

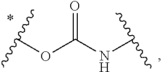

wherein R² is attached at the position denoted by *.

In some embodiments, Y is

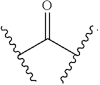 [SMILES: *C(*)=O]

In some embodiments, Y is

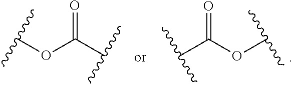 [SMILES: *OC(*)=O ; *OC(*)=O]

In some embodiments, Y is

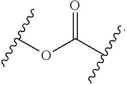 [SMILES: *OC(*)=O]

In some embodiments, Y is

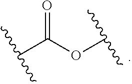 [SMILES: *OC(*)=O]

### R¹

In some embodiments, R¹ is —(CH₂)₁₋₆N(R^{a})₂ or —(CH₂)₁₋₆OH. In some embodiments, R¹ is —(CH₂)₁₋₆OH. In some embodiments, R¹ is —(CH₂)₁₋₆N(R^{a})₂. In some embodiments, R¹ is —(CH₂)₂N(R^{a})₂. In some embodiments, R¹ is —(CH₂)₃N(R^{a})₂. In some embodiments, R¹ is —(CH₂)₄N(R^{a})₂. In some embodiments, R¹ is —(CH₂)₁₋₆N(Me)₂. In some embodiments, R¹ is —(CH₂)₁₋₆N(Et)₂. In some embodiments, R¹ is —(CH₂)₁₋₆N(n-Pr)₂. In some embodiments, R¹ is —(CH₂)₁₋₆N(i-Pr)₂. In some embodiments, R¹ is —(CH₂)₂N(Me)₂. In some embodiments, R¹ is —(CH₂)₃N(Me)₂. In some embodiments, R¹ is —(CH₂)₄N(Me)₂. In some embodiments, R¹ is —(CH₂)₂N(Et)₂. In some embodiments, R¹ is —(CH₂)₃N(Et)₂. In some embodiments, R¹ is —(CH₂)₄N(Et)₂.

In some embodiments, R¹ is selected from the group consisting of

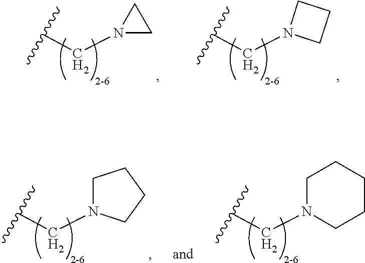

In some embodiments, R¹ is selected from the group consisting of

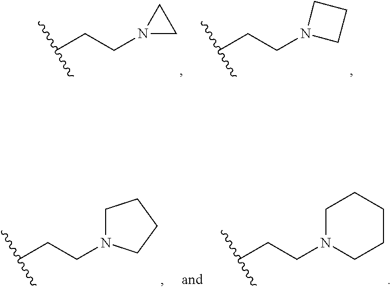

In some embodiments, R¹ is selected from the group consisting of

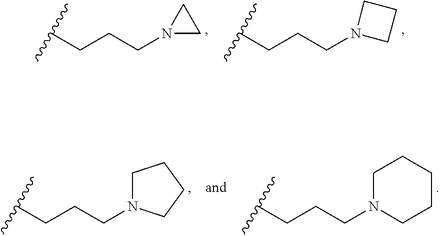

R² and R^{2′}

In some embodiments, R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—. In some embodiments, R² is optionally substituted C₁-C₃₂ alkyl or optionally substituted C₂-C₃₂ alkenyl, wherein 1-6 methylene units of R² are optionally replaced with a group each independently selected from cyclopropylene, —O—, —OC(O)—, and —C(O)O—. In some embodiments, R² is optionally substituted C₁-C₃₀ alkyl or optionally substituted C₂-C₃₀ alkenyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl, wherein 1-6 methylene units of R² are replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl. In some embodiments, R² is optionally substituted C₁₀-C₂₄ alkyl or optionally substituted C₁₀-C₂₄ alkenyl, wherein 1-6 methylene units of R² are replaced with —O—.

In some embodiments, R² is optionally substituted C₅-C₃₆ alkyl or optionally substituted C₅-C₃₆ alkenyl, wherein 2 methylene units of R² are replaced with —O— to form an acetal within R², and wherein 1-6 methylene units of R² are optionally replaced with a group each independently selected from cyclopropylene, —O—, —OC(O)—, and —C(O)O—; and R^{2′} is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—.

In some embodiments, R² is optionally substituted C₁₀-C₂₄ alkyl or optionally substituted C₁₀-C₂₄ alkenyl, wherein 2 methylene units of R² are replaced with —O— to form an acetal within R² and wherein 1-6 methylene units of R² are optionally replaced with a group each independently selected from cyclopropylene, —O—, —OC(O)—, and —C(O)O—; and R^{2′} is optionally substituted C₁₀-C₃₆ branched alkyl or optionally substituted C₁₀-C₃₆ alkenyl, wherein 1-6 methylene units of R² are optionally replaced with a group each independently selected from cyclopropylene, —O—, —OC(O)—, and —C(O)O—.

In some embodiments, R² is C₃-C₃₆ branched alkyl or optionally substituted C₃-C₃₆ branched alkenyl, wherein 1-6 methylene units of R² are optionally replaced with a group each independently selected from cyclopropylene and —O—; and R^{2′} is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)— and —C(O)O—. In some embodiments, R² is optionally substituted C₁₀-C₂₄ branched alkyl or optionally substituted C₁₀-C₂₄ branched alkenyl, wherein 1-3 methylene units of R² are optionally replaced with —O—; and R^{2′} is optionally substituted C₁₀-C₃₆ alkyl or optionally substituted C₁₀-C₃₆ alkenyl, wherein 1-6 methylene units of R² are optionally replaced with a group each independently selected from cyclopropylene, —O—, —OC(O)—, and —C(O)O—.

In some embodiments, R² and/or R^{2′} is

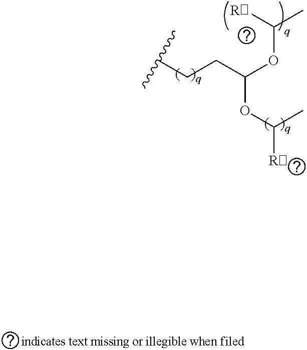

wherein each q is independently selected from 0-12 and each Rº is independently selected, and is as described and defined herein.

In some embodiments, R² and/or R^{2′} is

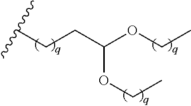

wherein each q is independently selected from 0-12.

In some embodiments, R² is optionally substituted C₅-C₃₆ alkyl or optionally substituted C₅-C₃₆ alkenyl, wherein 2 methylene units of R² are replaced with —O— to form an acetal within R², and wherein 1-6 methylene units of R² are optionally replaced with a group each independently selected from cyclopropylene, —O—, —OC(O)—, and —C(O)O—; R² is optionally substituted C₁-C₃₆ alkyl or optionally substituted C₅-C₃₆ alkenyl, wherein 1-6 methylene units of R^{2′} are optionally replaced with a group each independently selected from cyclopropylene, —O—, —OC(O)—, and —C(O)O—.

In some embodiments, R² is optionally substituted C₁₀-C₂₄ alkyl, wherein 2 methylene units of R² are replaced with —O— to form an acetal within R²; and R^{2′} is optionally substituted C₁₀-C₂₄ alkyl, wherein 2 methylene units of R² are replaced with —O— to form an acetal within R^{2′}.

In some embodiments, each q is independently selected from 0-6. In some embodiments, each q is independently selected from 0-8. In some embodiments, each q is independently selected from 0-10. In some embodiments, each q is independently selected from 0-12.

In some embodiments, R² is optionally substituted C₁₀-C₂₄ alkyl or optionally substituted C₁₀-C₂₄ alkenyl, wherein 2 methylene units of R² are replaced with —O— to form an acetal within R² and wherein 1-6 methylene units of R² are optionally replaced with a group each independently selected from cyclopropylene, —O—, —OC(O)—, and —C(O)O—; and R^{2′} is optionally substituted C₁₀-C₂₄ alkenyl, wherein 1-3 methylene units of R² are optionally replaced with —O—.

In some embodiments, R² is selected from the group consisting of

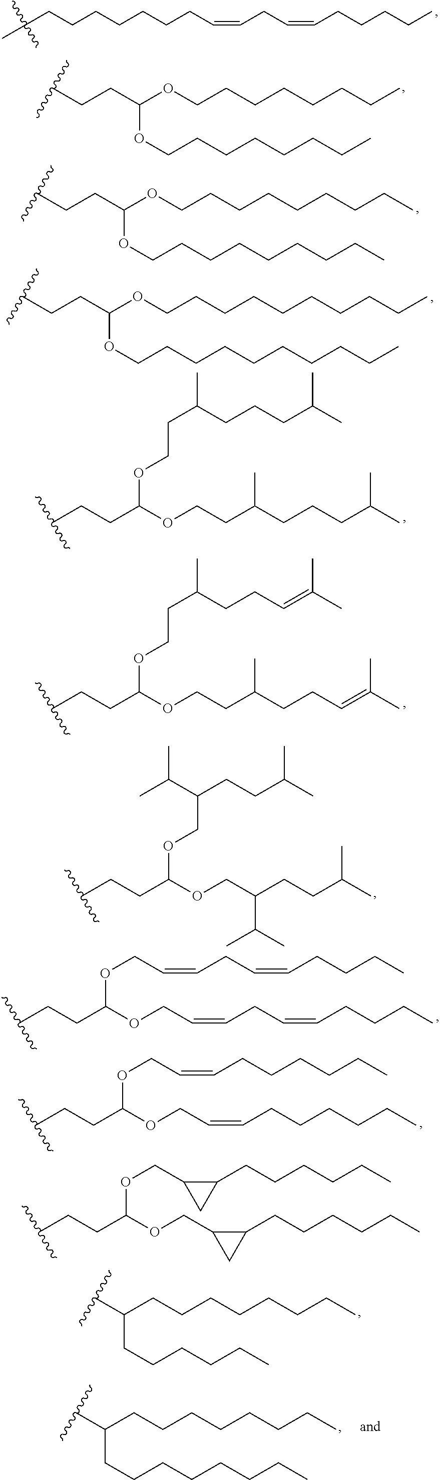

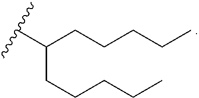 [SMILES: *C(CCCCC)CCCCC]

In some embodiments, R² is

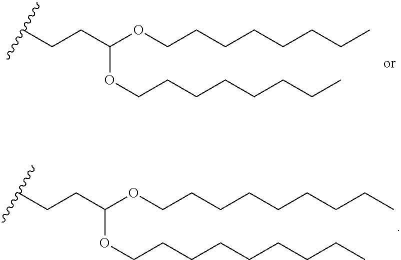 [SMILES: *CCC(OCCCCCCCC)OCCCCCCCC ; *CCC(OCCCCCCCCC)OCCCCCCCCC]

In some embodiments R² is

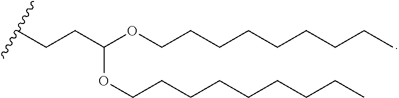 [SMILES: *CCC(OCCCCCCCCC)OCCCCCCCCC]

In some embodiments, R² is

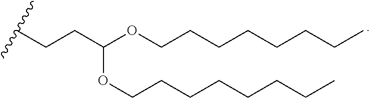 [SMILES: *CCC(OCCCCCCCC)OCCCCCCCC]

In some embodiments, R² is selected from the group consisting of

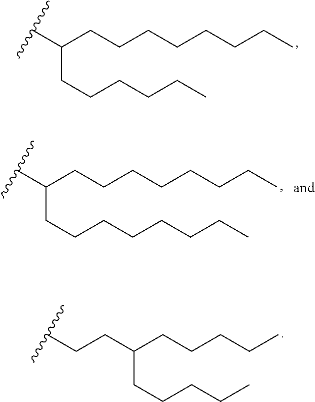 [SMILES: *C(CCCCCC)CCCCCCCC ; *C(CCCCCCCC)CCCCCCCC ; *CCC(CCCCC)CCCCC]

In some embodiments, R^{2′} is optionally substituted C₁-C₃₆ alkyl, wherein 1-6 methylene units of R^{2′} are optionally replaced with a group each independently selected from cyclopropylene, —O—, —OC(O)—, and —C(O)O—. In some embodiments, R^{2′} is optionally substituted C₁-C₃₂ alkyl, wherein 1-6 methylene units of R^{2′} are optionally replaced with a group each independently selected from cyclopropylene, —O—, —OC(O)—, and —C(O)O—. In some embodiments, R^{2′} is optionally substituted C₁-C₃₀ alkyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl, wherein 1-6 methylene units of R^{2′} are optionally replaced with a group each independently selected from cyclopropylene, —O—, —OC(O)—, and —C(O)O—. In some embodiments, R^{2′} is optionally substituted C₁-C₂₄ alkyl, wherein 1-6 methylene units of R^{2′} are replaced with a group each independently selected from —O—, —OC(O)—, and —C(O)O—. In some embodiments, R² is optionally substituted C₁-C₂₄ alkyl. In some embodiments, R^{2′} is optionally substituted C₁₀-C₂₄ alkyl, wherein 1-6 methylene units of R^{2′} are replaced with —O—.

In some embodiments, R^{2′} is selected from the group consisting of

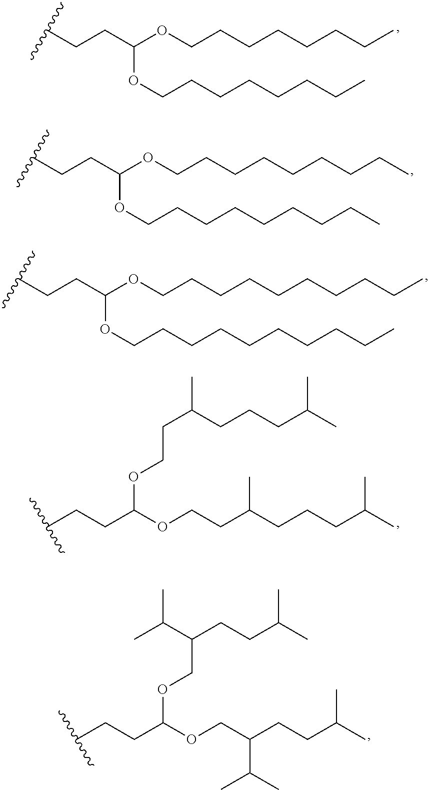

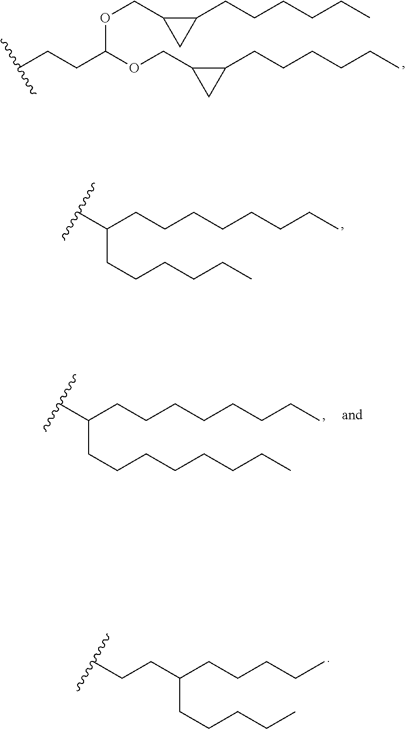 [SMILES: *CCC(OCC1CC1CCCCCC)OCC1CC1CCCCCC ; *C(CCCCCC)CCCCCCCC ; *C(CCCCCCCC)CCCCCCCC ; *CCC(CCCCC)CCCCC]

In some embodiments, R² and R^{2′} are each independently selected from the group consisting of

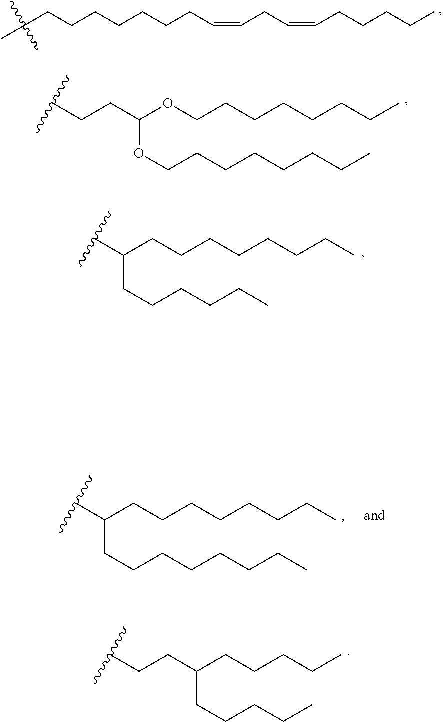 [SMILES: *CCCCCCC/C=C\C/C=C\CCCCC ; *CCC(OCCCCCCCC)OCCCCCCCC ; *C(CCCCCC)CCCCCCCC ; *C(CCCCCCCC)CCCCCCCC ; *CCC(CCCCC)CCCCC]

In some embodiments, R² is selected from the group consisting of

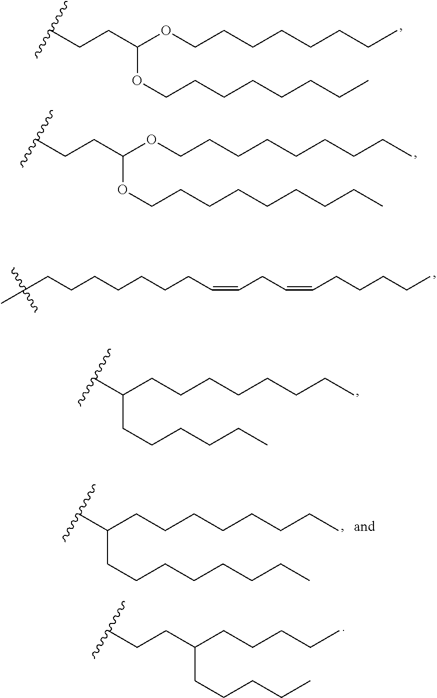

In some embodiments, R² is

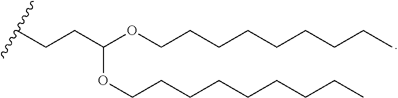 [SMILES: *CCC(OCCCCCCCCC)OCCCCCCCCC]

In some embodiments, R² is

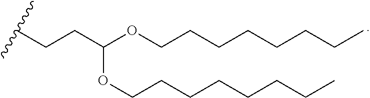 [SMILES: *CCC(OCCCCCCCC)OCCCCCCCC]

In some embodiments, R² is selected from the group consisting of

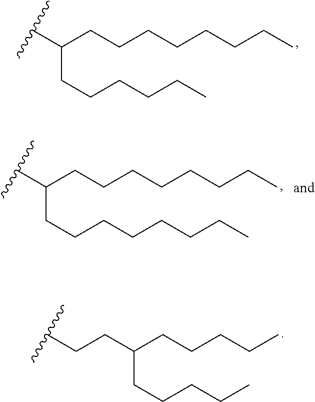 [SMILES: *C(CCCCCC)CCCCCCCC ; *C(CCCCCCCC)CCCCCCCC ; *CCC(CCCCC)CCCCC]

In some embodiments, R² is selected from the group consisting of

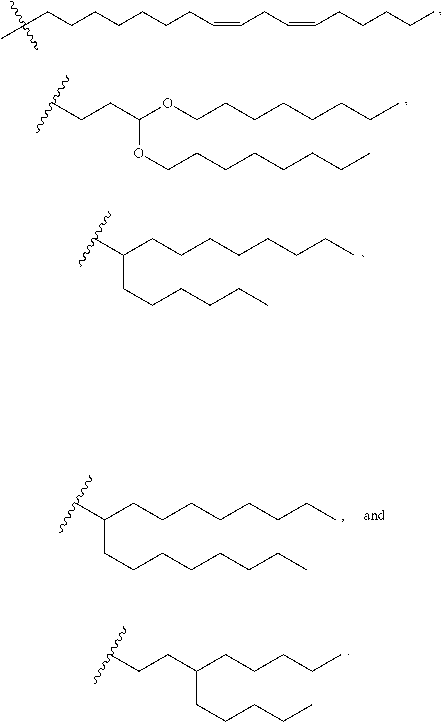 [SMILES: *CCCCCCC/C=C\C/C=C\CCCCC ; *CCC(OCCCCCCCC)OCCCCCCCC ; *C(CCCCCC)CCCCCCCC ; *C(CCCCCCCC)CCCCCCCC ; *CCC(CCCCC)CCCCC]

In some embodiments, R² is selected from the group consisting of

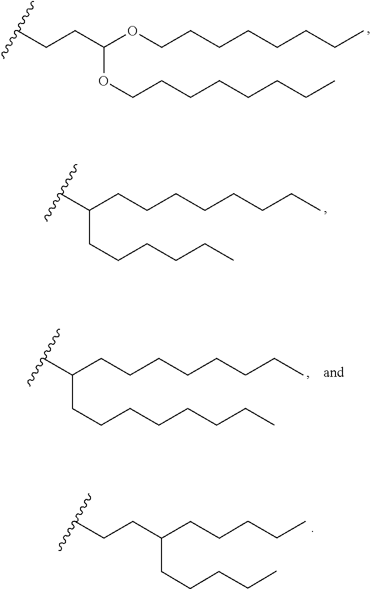 [SMILES: *CCC(OCCCCCCCC)OCCCCCCCC ; *C(CCCCCC)CCCCCCCC ; *C(CCCCCCCC)CCCCCCCC ; *CCC(CCCCC)CCCCC]

In some embodiments, the present disclosure includes a compound selected from any lipid in Table (IV) below or a pharmaceutically acceptable salt thereof:

Table (IV)
Non-Limiting Examples of Ionizable Lipids

| | Compound |
|---|---|
| Structure | No. |
| 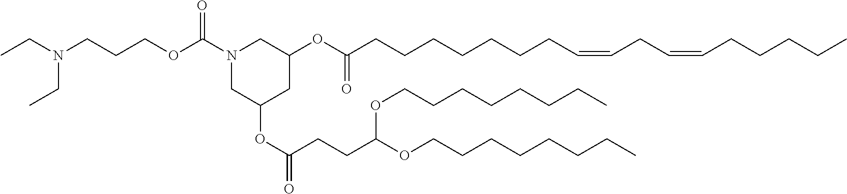 [SMILES: CCCCC/C=C\C/C=C\CCCCCCCC(=O)OC1CC(OC(=O)CCC(OCCCCCCCC)OCCCCCCCC)CN(C(=O)OCCCN(CC)CC)C1] | CX-1 |
| 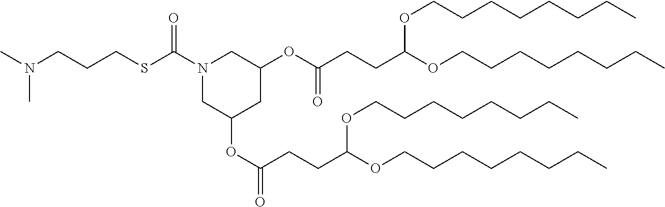 [SMILES: CCCCCCCCOC(CCC(=O)OC1CC(OC(=O)CCC(OCCCCCCCC)OCCCCCCCC)CN(C(=O)SCCCN(C)C)C1)OCCCCCCCC] | CX-2 |
| 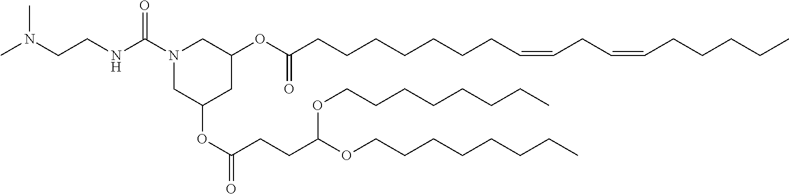 [SMILES: CCCCC/C=C\C/C=C\CCCCCCCC(=O)OC1CC(OC(=O)CCC(OCCCCCCCC)OCCCCCCCC)CN(C(=O)NCCN(C)C)C1] | CX-3 |
| 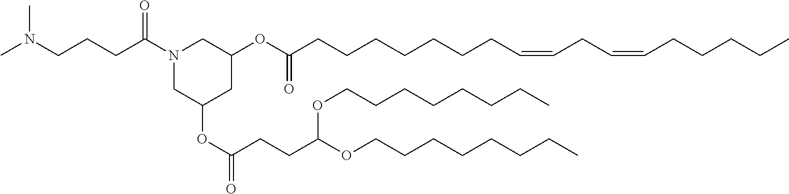 [SMILES: CCCCC/C=C\C/C=C\CCCCCCCC(=O)OC1CC(OC(=O)CCC(OCCCCCCCC)OCCCCCCCC)CN(C(=O)CCCN(C)C)C1] | CX-4 |
| 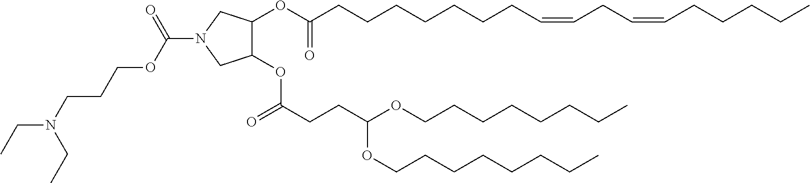 [SMILES: CCCCC/C=C\C/C=C\CCCCCCCC(=O)OC1CN(C(=O)OCCCN(CC)CC)CC1OC(=O)CCC(OCCCCCCCC)OCCCCCCCC] | CX-5 |
| 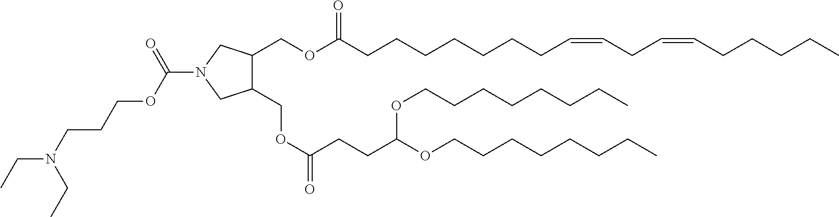 [SMILES: CCCCC/C=C\C/C=C\CCCCCCCC(=O)OCC1CN(C(=O)OCCCN(CC)CC)CC1COC(=O)CCC(OCCCCCCCC)OCCCCCCCC] | CX-6 |
| 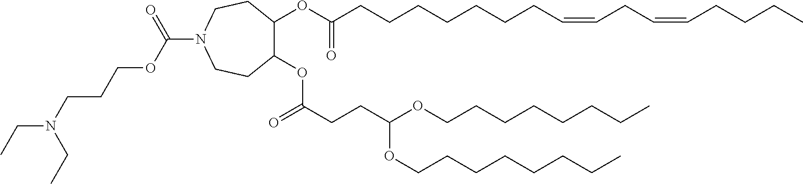 [SMILES: CCCC/C=C\C/C=C\CCCCCCCC(=O)OC1CCN(C(=O)OCCCN(CC)CC)CCC1OC(=O)CCC(OCCCCCCCC)OCCCCCCCC] | CX-7 |
| 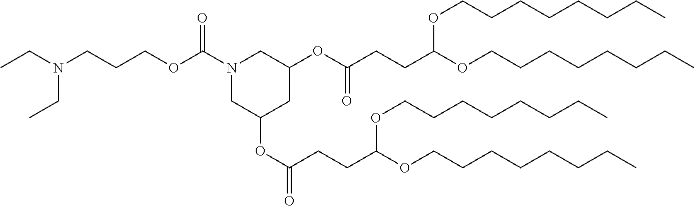 [SMILES: CCCCCCCCOC(CCC(=O)OC1CC(OC(=O)CCC(OCCCCCCCC)OCCCCCCCC)CN(C(=O)OCCCN(CC)CC)C1)OCCCCCCCC] | CX-8 |
| 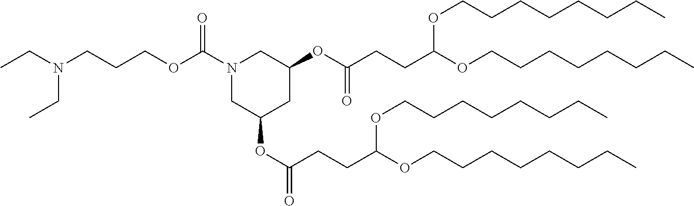 [SMILES: CCCCCCCCOC(CCC(=O)O[C@@H]1C[C@H](OC(=O)CCC(OCCCCCCCC)OCCCCCCCC)CN(C(=O)OCCCN(CC)CC)C1)OCCCCCCCC] | CX-8a |
| 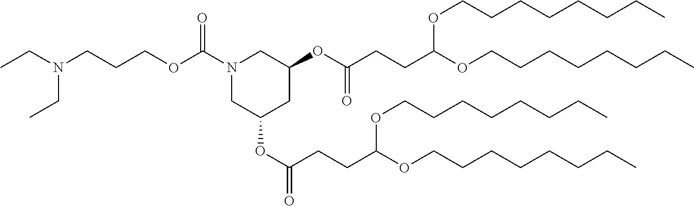 [SMILES: CCCCCCCCOC(CCC(=O)O[C@H]1C[C@H](OC(=O)CCC(OCCCCCCCC)OCCCCCCCC)CN(C(=O)OCCCN(CC)CC)C1)OCCCCCCCC] | CX-8b |
| 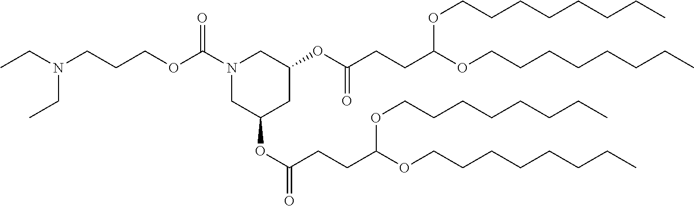 [SMILES: CCCCCCCCOC(CCC(=O)O[C@@H]1C[C@@H](OC(=O)CCC(OCCCCCCCC)OCCCCCCCC)CN(C(=O)OCCCN(CC)CC)C1)OCCCCCCCC] | CX-8c |
| 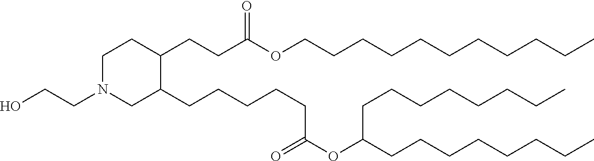 [SMILES: CCCCCCCCCCCOC(=O)CCC1CCN(CCO)CC1CCCCCC(=O)OC(CCCCCCCC)CCCCCCCC] | CX-9 |
| 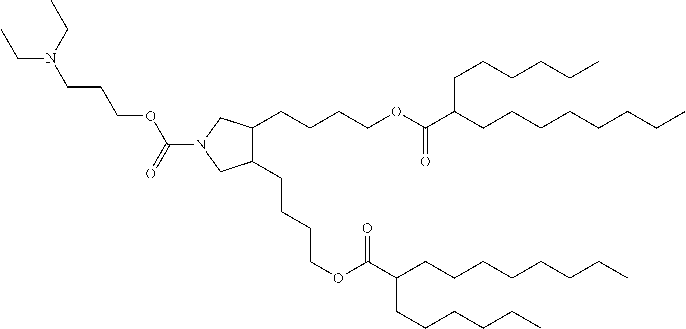 [SMILES: CCCCCCCCC(CCCCCC)C(=O)OCCCCC1CN(C(=O)OCCCN(CC)CC)CC1CCCCOC(=O)C(CCCCCC)CCCCCCCC] | CX-10 |
| 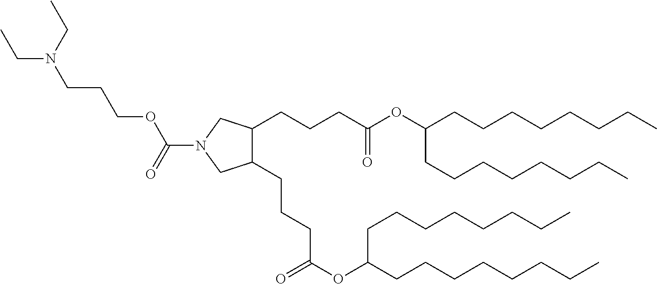 [SMILES: CCCCCCCCC(CCCCCCCC)OC(=O)CCCC1CN(C(=O)OCCCN(CC)CC)CC1CCCC(=O)OC(CCCCCCCC)CCCCCCCC] | CX-11 |
| 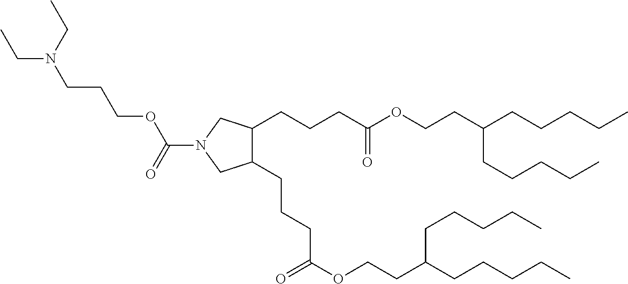 [SMILES: CCCCCC(CCCCC)CCOC(=O)CCCC1CN(C(=O)OCCCN(CC)CC)CC1CCCC(=O)OCCC(CCCCC)CCCCC] | CX-12 |
| 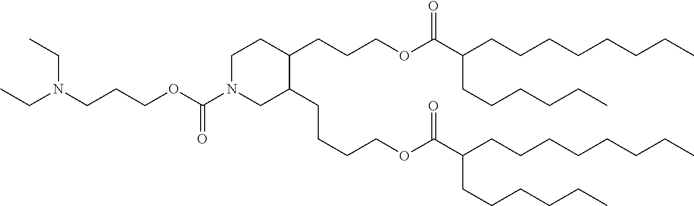 [SMILES: CCCCCCCCC(CCCCCC)C(=O)OCCCCC1CN(C(=O)OCCCN(CC)CC)CCC1CCCOC(=O)C(CCCCCC)CCCCCCCC] | CX-13 |
| 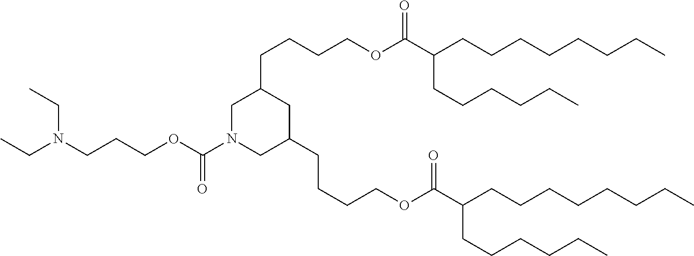 [SMILES: CCCCCCCCC(CCCCCC)C(=O)OCCCCC1CC(CCCCOC(=O)C(CCCCCC)CCCCCCCC)CN(C(=O)OCCCN(CC)CC)C1] | CX-14 |
| 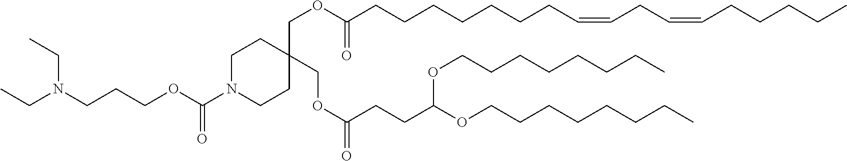 [SMILES: CCCCC/C=C\C/C=C\CCCCCCCC(=O)OCC1(COC(=O)CCC(OCCCCCCCC)OCCCCCCCC)CCN(C(=O)OCCCN(CC)CC)CC1] | CX-15 |
| 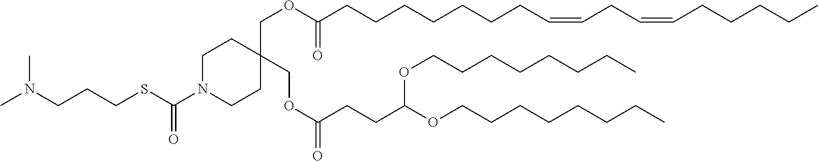 [SMILES: CCCCC/C=C\C/C=C\CCCCCCCC(=O)OCC1(COC(=O)CCC(OCCCCCCCC)OCCCCCCCC)CCN(C(=O)SCCCN(C)C)CC1] | CX-16 |
| 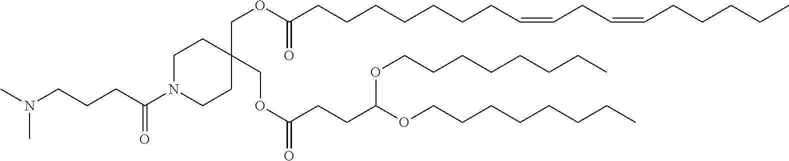 [SMILES: CCCCC/C=C\C/C=C\CCCCCCCC(=O)OCC1(COC(=O)CCC(OCCCCCCCC)OCCCCCCCC)CCN(C(=O)CCCN(C)C)CC1] | CX-17 |
| 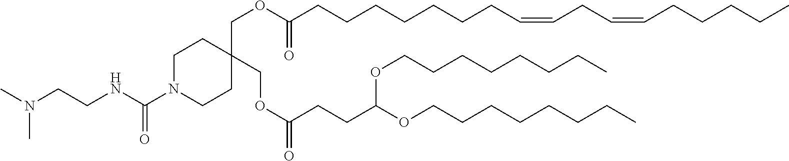 [SMILES: CCCCC/C=C\C/C=C\CCCCCCCC(=O)OCC1(COC(=O)CCC(OCCCCCCCC)OCCCCCCCC)CCN(C(=O)NCCN(C)C)CC1] | CX-18 |
| 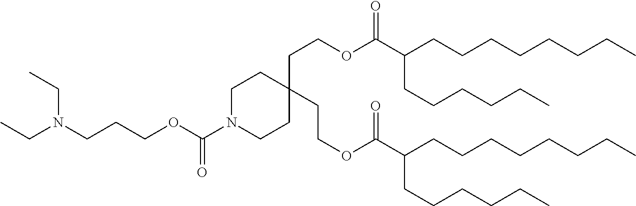 [SMILES: CCCCCCCCC(CCCCCC)C(=O)OCCC1(CCOC(=O)C(CCCCCC)CCCCCCCC)CCN(C(=O)OCCCN(CC)CC)CC1] | CX-19 |
| 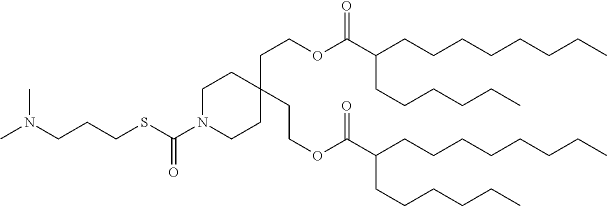 [SMILES: CCCCCCCCC(CCCCCC)C(=O)OCCC1(CCOC(=O)C(CCCCCC)CCCCCCCC)CCN(C(=O)SCCCN(C)C)CC1] | CX-20 |
| 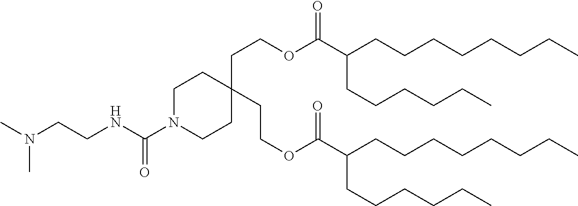 [SMILES: CCCCCCCCC(CCCCCC)C(=O)OCCC1(CCOC(=O)C(CCCCCC)CCCCCCCC)CCN(C(=O)NCCN(C)C)CC1] | CX-21 |
| 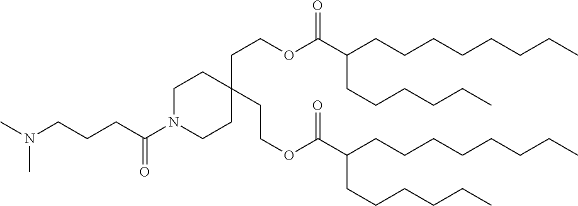 [SMILES: CCCCCCCCC(CCCCCC)C(=O)OCCC1(CCOC(=O)C(CCCCCC)CCCCCCCC)CCN(C(=O)CCCN(C)C)CC1] | CX-22 |
| 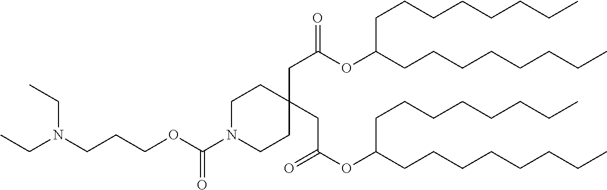 [SMILES: CCCCCCCCC(CCCCCCCC)OC(=O)CC1(CC(=O)OC(CCCCCCCC)CCCCCCCC)CCN(C(=O)OCCCN(CC)CC)CC1] | CX-23 |
| 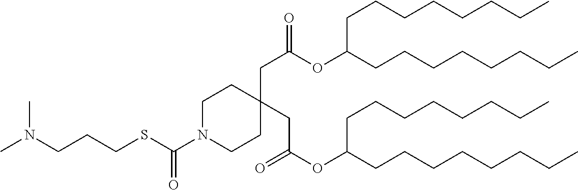 [SMILES: CCCCCCCCC(CCCCCCCC)OC(=O)CC1(CC(=O)OC(CCCCCCCC)CCCCCCCC)CCN(C(=O)SCCCN(C)C)CC1] | CX-24 |
| 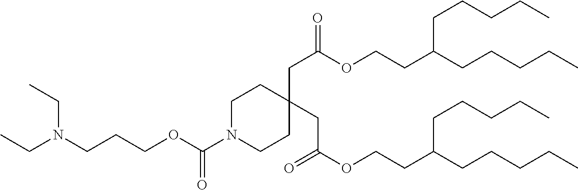 [SMILES: CCCCCC(CCCCC)CCOC(=O)CC1(CC(=O)OCCC(CCCCC)CCCCC)CCN(C(=O)OCCCN(CC)CC)CC1] | CX-25 |
| 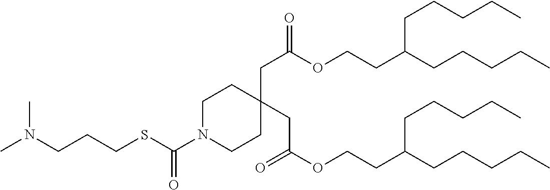 [SMILES: CCCCCC(CCCCC)CCOC(=O)CC1(CC(=O)OCCC(CCCCC)CCCCC)CCN(C(=O)SCCCN(C)C)CC1] | CX-26 |
| 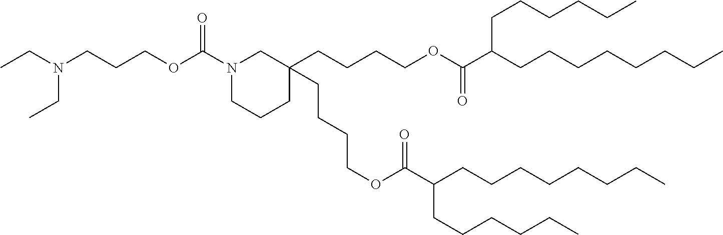 [SMILES: CCCCCCCCC(CCCCCC)C(=O)OCCCCC1(CCCCOC(=O)C(CCCCCC)CCCCCCCC)CCCN(C(=O)OCCCN(CC)CC)C1] | CX-27 |
| 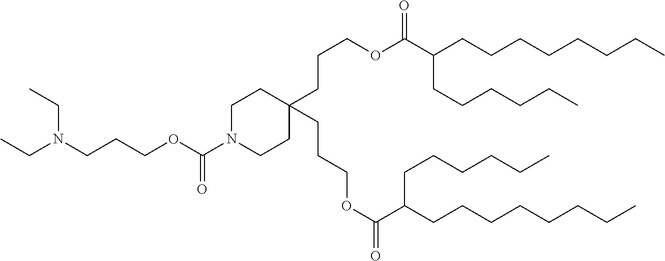 [SMILES: CCCCCCCCC(CCCCCC)C(=O)OCCCC1(CCCOC(=O)C(CCCCCC)CCCCCCCC)CCN(C(=O)OCCCN(CC)CC)CC1] | CX-28 |
| 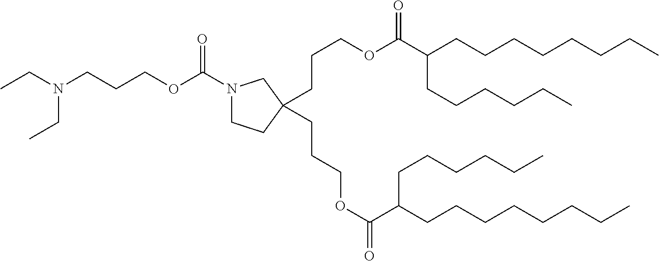 [SMILES: CCCCCCCCC(CCCCCC)C(=O)OCCCC1(CCCOC(=O)C(CCCCCC)CCCCCCCC)CCN(C(=O)OCCCN(CC)CC)C1] | CX-29 |
| 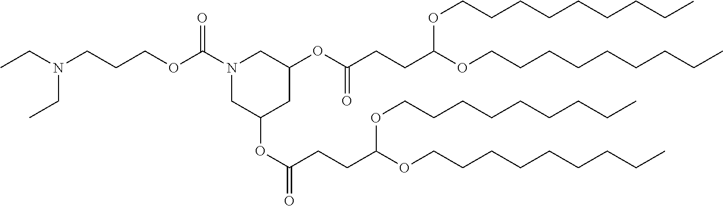 [SMILES: CCCCCCCCCOC(CCC(=O)OC1CC(OC(=O)CCC(OCCCCCCCCC)OCCCCCCCCC)CN(C(=O)OCCCN(CC)CC)C1)OCCCCCCCCC] | CX-30 |
| 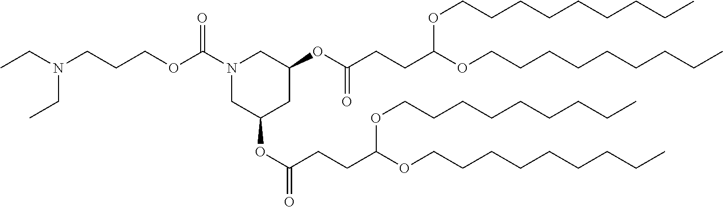 [SMILES: CCCCCCCCCOC(CCC(=O)O[C@@H]1C[C@H](OC(=O)CCC(OCCCCCCCCC)OCCCCCCCCC)CN(C(=O)OCCCN(CC)CC)C1)OCCCCCCCCC] | CX-30a |
| 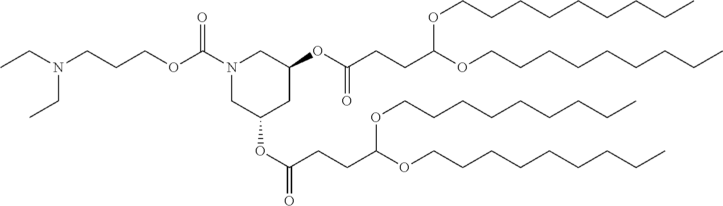 [SMILES: CCCCCCCCCOC(CCC(=O)O[C@H]1C[C@H](OC(=O)CCC(OCCCCCCCCC)OCCCCCCCCC)CN(C(=O)OCCCN(CC)CC)C1)OCCCCCCCCC] | CX-30b |
| 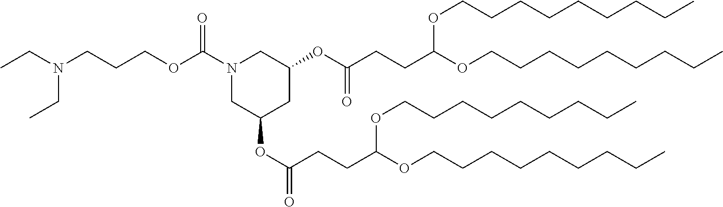 [SMILES: CCCCCCCCCOC(CCC(=O)O[C@@H]1C[C@@H](OC(=O)CCC(OCCCCCCCCC)OCCCCCCCCC)CN(C(=O)OCCCN(CC)CC)C1)OCCCCCCCCC] | CX-30c |

In some embodiments, lipids of the present disclosure comprise a heterocyclic core, wherein the heteroatom is nitrogen. In some embodiments, the heterocyclic core comprises pyrrolidine or a derivative thereof. In some embodiments, the heterocyclic core comprises piperidine or a derivative thereof.

In some embodiments, a compound of the present disclosure is represented by Formula (CZ-T)

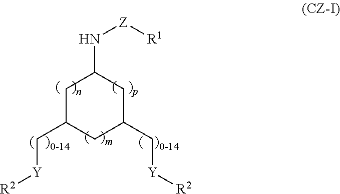

or a pharmaceutically acceptable salt thereof,
wherein
Z is selected from the group consisting of a bond,

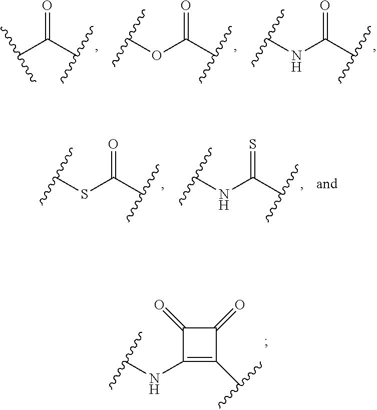

each Y is independently selected from the group consisting of,

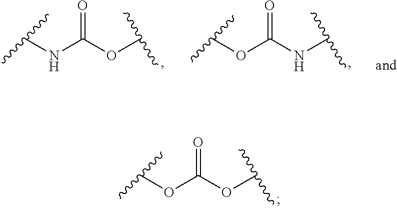 [SMILES: *NC(=O)O* ; *NC(=O)O* ; *OC(=O)O*]

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
two R^{a} are taken together, with the nitrogen on which they are attached, to form an optionally substituted 4-7 membered heterocyclyl ring;
m is 0, 1, or 2;
n is 1 or 2; and
p is 1 or 2.

In some embodiments, a compound of the present disclosure is represented by Formula (CZ-I-a), (CZ-I-b), (CZ-I-c), or (CZ-I-d)

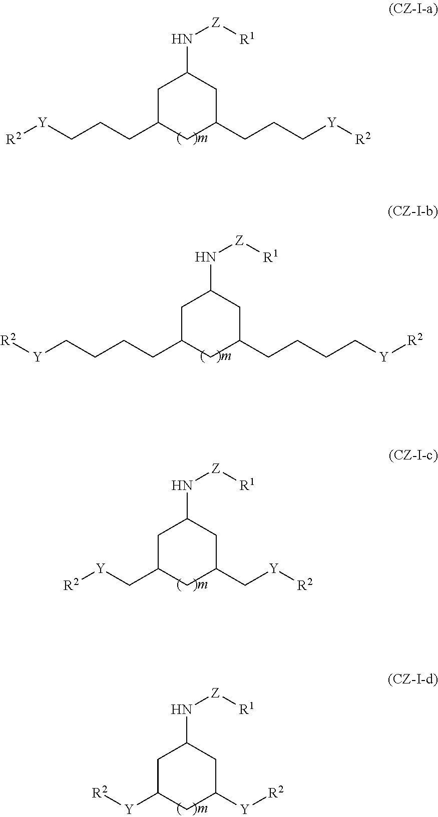

or a pharmaceutically acceptable salt thereof.

In some embodiments, a compound of the present disclosure is represented by Formula (CZ-I-e) or (CZ-I-f)

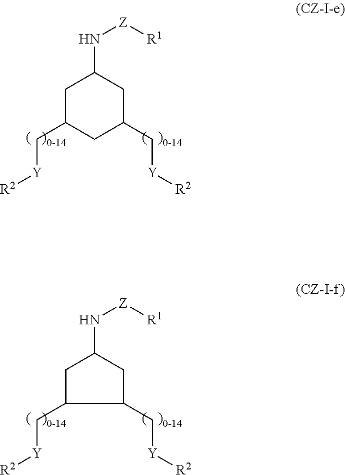

or a pharmaceutically acceptable salt thereof.

In some embodiments, a compound of the present disclosure is represented by Formula (CZ-I-g)

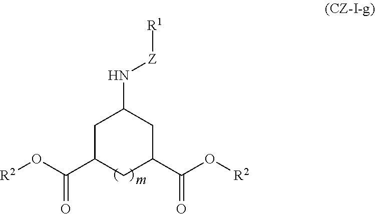

or a pharmaceutically acceptable salt thereof.

In some embodiments, a compound of the present disclosure is represented by Formula (CZ-II)

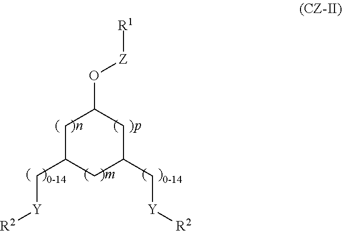

or a pharmaceutically acceptable salt thereof,
wherein
Z is selected from the group consisting of a bond,

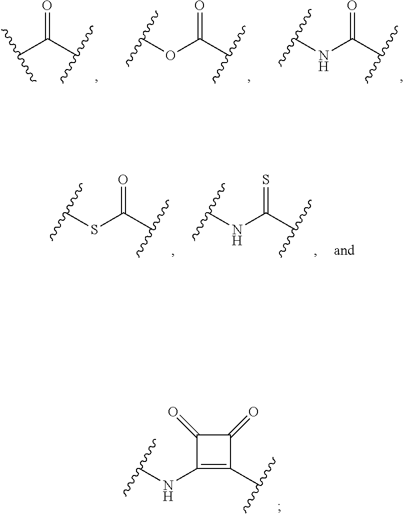

each Y is independently selected from the group consisting of,

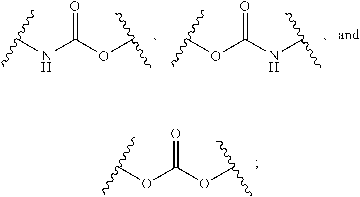 [SMILES: *NC(=O)O* ; *NC(=O)O* ; *OC(=O)O*]

R¹ is —(CH₂)₁₋₆N(R^{a})₂;
each R² is independently optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—;
each R^{a} is independently optionally substituted C₁-C₆ alkyl; or
   two R^{a} are taken together, with the nitrogen on which they are attached, to form an optionally substituted 4-7 membered heterocyclyl ring;
m is 0, 1, or 2;
n is 1 or 2; and
p is 1 or 2.
   or a pharmaceutically acceptable salt thereof.

In some embodiments, a compound of the present disclosure is represented by Formula (CZ-II-a), (CZ-II-b), (CZ-II-c) or (CZ-II-d):

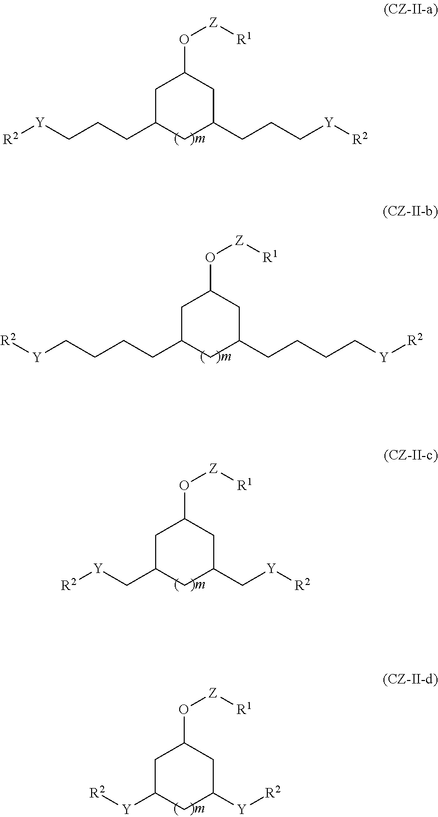

or a pharmaceutically acceptable salt thereof.

In some embodiments, a compound of the present disclosure is represented by Formula (CZ-II-e)

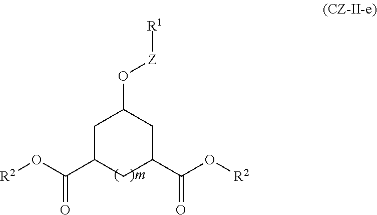

or a pharmaceutically acceptable salt thereof.

### Z

In some embodiments, Z is selected from the group consisting of a bond,

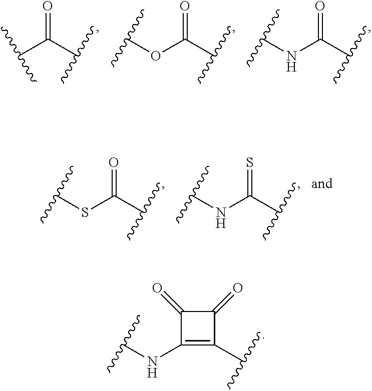

In some embodiments, Z is selected from the group consisting of

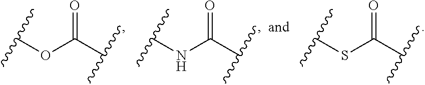 [SMILES: *OC(*)=O ; *NC(*)=O ; *SC(*)=O]

In some embodiments, Z is

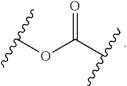 [SMILES: *OC(*)=O]

In some embodiments, Z is selected from the group consisting of a bond,

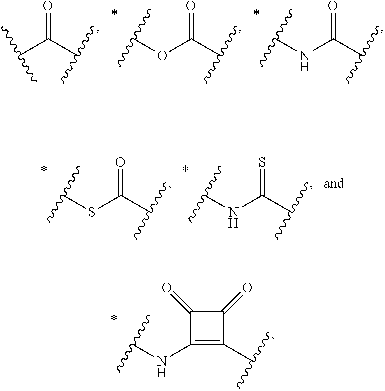

wherein R¹ is attached at the position denoted by *.

In some embodiments, Z is selected from the group consisting of

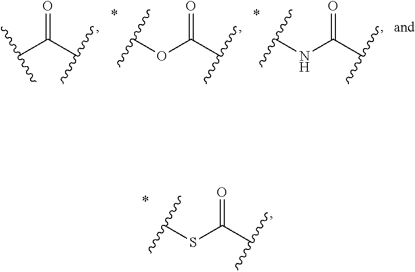

wherein R¹ is attached at the position denoted by *.

In some embodiments, Z is

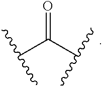 [SMILES: *C(*)=O]

In some embodiments, Z is

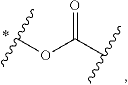

wherein R¹ is attached at the position denoted by *.

In some embodiments, Z is

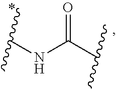

wherein R¹ is attached at the position denoted by *.

In some embodiments, Z is

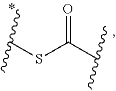

wherein R¹ is attached at the position denoted by *.

In some embodiments, Z is

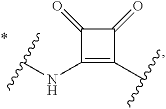

wherein R¹ is attached at the position denoted by *.

### Y

In some embodiments, Y is selected from the group consisting of,

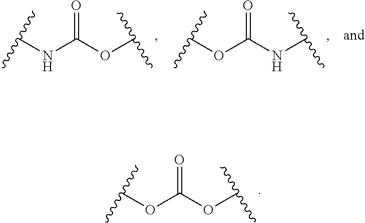 [SMILES: *NC(=O)O* ; *NC(=O)O* ; *OC(=O)O*]

In some embodiments, Y is

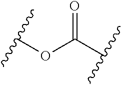 [SMILES: *OC(*)=O]

or.

In some embodiments, Y is selected from the group consisting of, H

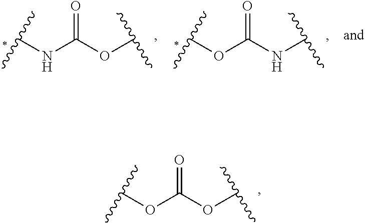

wherein R² is attached at the position denoted by *.

In some embodiments, Y is, wherein R² is attached at the position denoted by *. In some embodiments, Y is, wherein R² is attached at the position denoted by *.

In some embodiments, Y is

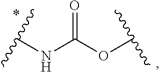

wherein R² is attached at the position denoted by *.

In some embodiments, Y is

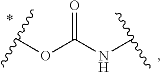

wherein R² is attached at the position denoted by *.

In some embodiments, Y is

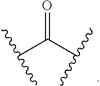 [SMILES: *C(*)=O]

In some embodiments, Y is

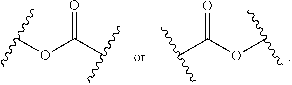 [SMILES: *OC(*)=O ; *OC(*)=O]

In some embodiments, Y is

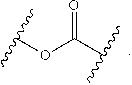 [SMILES: *OC(*)=O]

In some embodiments, Y is

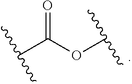 [SMILES: *OC(*)=O]

### R¹

In some embodiments, R¹ is —(CH₂)₁₋₆N(R^{a})₂. In some embodiments, R¹ is —(CH₂)₂N(R^{a})₂. In some embodiments, R¹ is —(CH₂)₃N(R^{a})₂. In some embodiments, R¹ is —(CH₂)₄N(R^{a})₂. In some embodiments, R¹ is —(CH₂)₁₋₆N(Me)₂. In some embodiments, R¹ is —(CH₂)₁₋₆N(Et)₂. In some embodiments, R¹ is —(CH₂)₁₋₆N(n-Pr)₂. In some embodiments, R¹ is —(CH₂)₁₋₆N(CZ—I—Pr)₂. In some embodiments, R¹ is —(CH₂)₂N(Me)₂. In some embodiments, R¹ is —(CH₂)₃N(Me)₂. In some embodiments, R¹ is —(CH₂)₄N(Me)₂. In some embodiments, R¹ is —(CH₂)₂N(Et)₂. In some embodiments, R¹ is —(CH₂)₃N(Et)₂. In some embodiments, R¹ is —(CH₂)₄N(Et)₂.

In some embodiments, R¹ is selected from the group consisting of

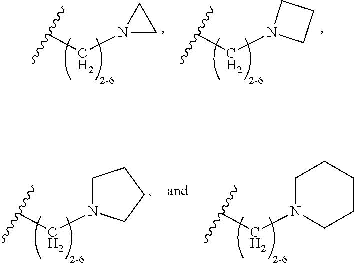

In some embodiments, R¹ is selected from the group consisting of

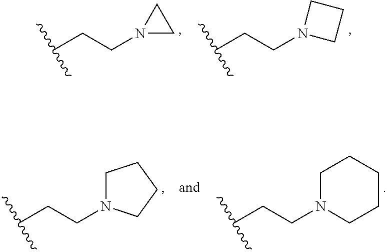

In some embodiments, R¹ is selected from the group consisting of

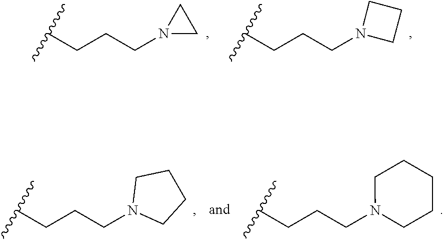 [SMILES: *CCCN1CC1 ; *CCCN1CCC1 ; *CCCN1CCCC1 ; *CCCN1CCCCC1]

### R²

In some embodiments, R² is optionally substituted C₁-C₃₆ alkyl or optionally substituted C₂-C₃₆ alkenyl, wherein 1-6 methylene units of R² are optionally replaced with a group each independently selected from cyclopropylene, —O—, —OC(O)—, and —C(O)O—. In some embodiments, R² is optionally substituted C₁-C₃₂ alkyl or optionally substituted C₂-C₃₂ alkenyl, wherein 1-6 methylene units of R² are optionally replaced with a group each independently selected from cyclopropylene, —O—, —OC(O)—, and —C(O)O—. In some embodiments, R² is optionally substituted C₁-C₃₀ alkyl or optionally substituted C₂-C₃₀ alkenyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl, wherein 1-6 methylene units of R² are optionally replaced with a group each independently selected from cyclopropylene, —O—, —OC(O)—, and —C(O)O—. In some embodiments, R² is optionally substituted C₁-C₂₄ alkyl or optionally substituted C₂-C₂₄ alkenyl, wherein 1-6 methylene units of R² are replaced with a group each independently selected from —O—, —OC(O)—, and —C(O)O—. In some embodiments, R² is optionally substituted C₁-C₂₄ alkyl or optionally substituted C₂-C₂₄ alkenyl. In some embodiments, R² is optionally substituted C₁₀-C₂₄ alkyl or optionally substituted C₁₀-C₂₄ alkenyl, wherein 1-6 methylene units of R² are replaced with —O—.

In some embodiments, R² is

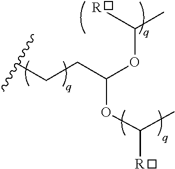

wherein each q is independently selected from 0-12 and each Rº is independently selected and defined herein.

In some embodiments, R² is

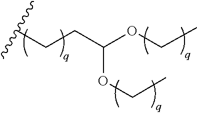

wherein each q is independently selected from 0-12.

In some embodiments, each q is independently selected from 0-6. In some embodiments, each q is independently selected from 0-8. In some embodiments, each q is independently selected from 0-10. In some embodiments, each q is independently selected from 0-12.

In some embodiments, R² is selected from the group consisting of

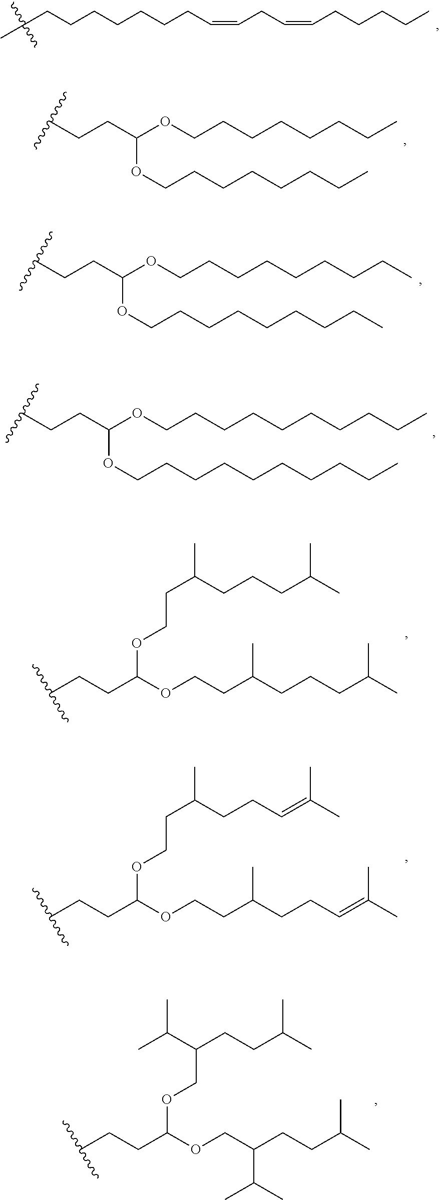

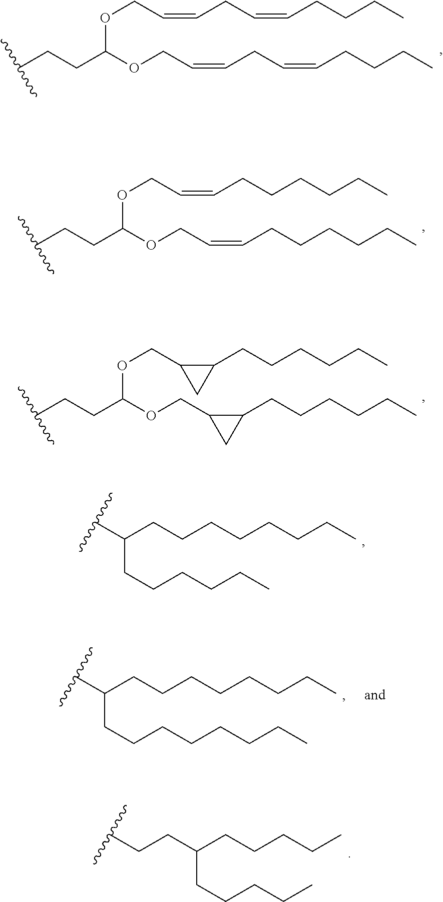 [SMILES: *CCC(OC/C=C\C/C=C\CCCC)OC/C=C\C/C=C\CCCC ; *CCC(OC/C=C\CCCCCC)OC/C=C\CCCCCC ; *CCC(OCC1CC1CCCCCC)OCC1CC1CCCCCC ; *C(CCCCCC)CCCCCCCC ; *C(CCCCCCCC)CCCCCCCC ; *CCC(CCCCC)CCCCC]

In some embodiments, R² is selected from the group consisting of

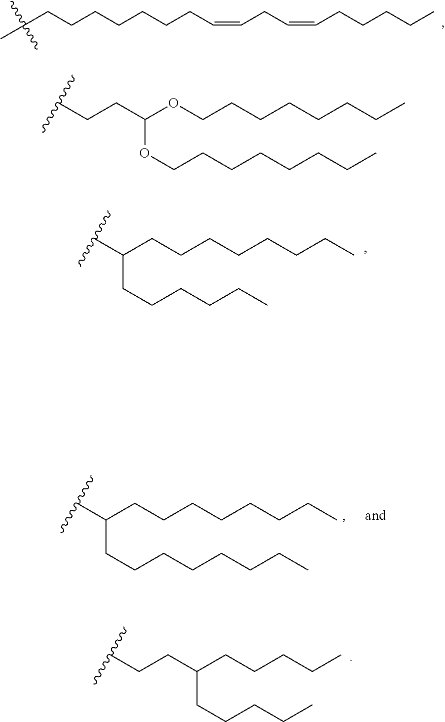 [SMILES: *CCCCCCC/C=C\C/C=C\CCCCC ; *CCC(OCCCCCCCC)OCCCCCCCC ; *C(CCCCCC)CCCCCCCC ; *C(CCCCCCCC)CCCCCCCC ; *CCC(CCCCC)CCCCC]

In some embodiments, the present disclosure includes a compound selected from any lipid in Table (V) below or a pharmaceutically acceptable salt thereof:

TABLE (V)
Non-Limiting Examples of Ionizable Lipids

| | Compound |
|---|---|
| Structure | No. |
| 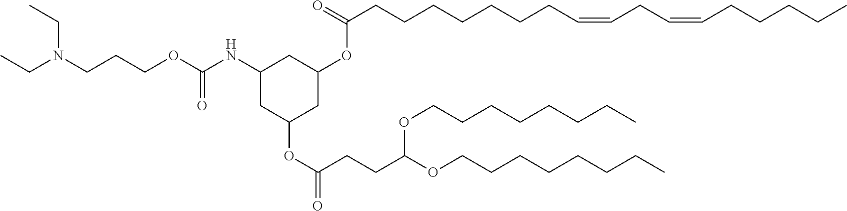 [SMILES: CCCCC/C=C\C/C=C\CCCCCCCC(=O)OC1CC(NC(=O)OCCCN(CC)CC)CC(OC(=O)CCC(OCCCCCCCC)OCCCCCCCC)C1] | CZ-1 |
| 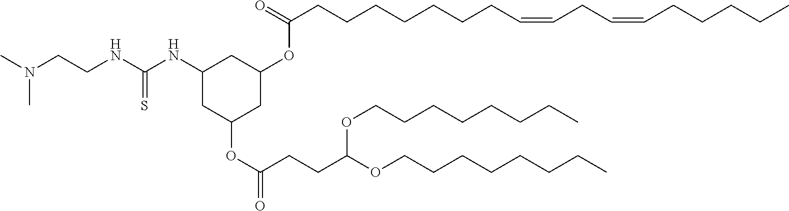 [SMILES: CCCCC/C=C\C/C=C\CCCCCCCC(=O)OC1CC(NC(=S)NCCN(C)C)CC(OC(=O)CCC(OCCCCCCCC)OCCCCCCCC)C1] | CZ-2 |
| 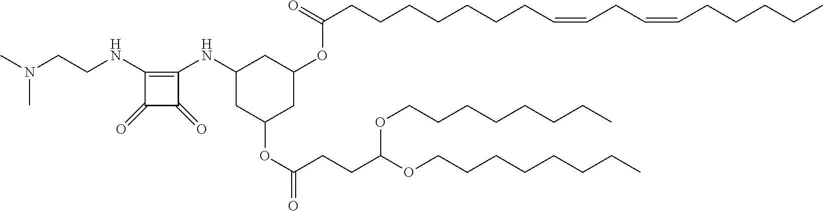 [SMILES: CCCCC/C=C\C/C=C\CCCCCCCC(=O)OC1CC(Nc2c(NCCN(C)C)c(=O)c2=O)CC(OC(=O)CCC(OCCCCCCCC)OCCCCCCCC)C1] | CZ-3 |
| 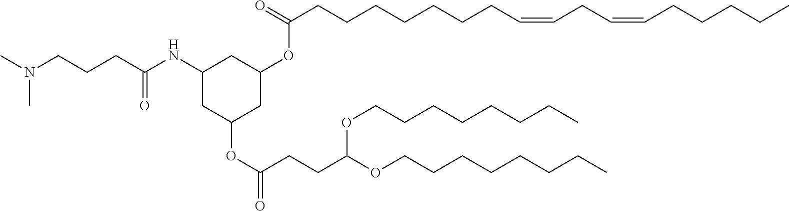 [SMILES: CCCCC/C=C\C/C=C\CCCCCCCC(=O)OC1CC(NC(=O)CCCN(C)C)CC(OC(=O)CCC(OCCCCCCCC)OCCCCCCCC)C1] | CZ-4 |
| 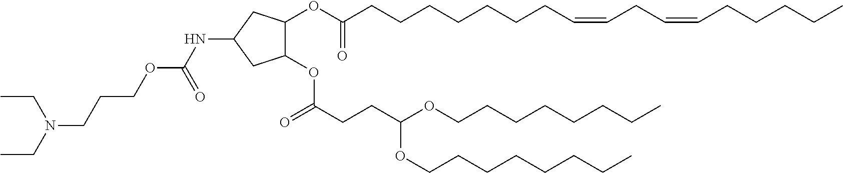 [SMILES: CCCCC/C=C\C/C=C\CCCCCCCC(=O)OC1CC(NC(=O)OCCCN(CC)CC)CC1OC(=O)CCC(OCCCCCCCC)OCCCCCCCC] | CZ-5 |
| 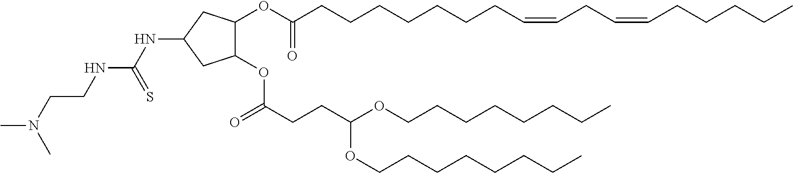 [SMILES: CCCCC/C=C\C/C=C\CCCCCCCC(=O)OC1CC(NC(=S)NCCN(C)C)CC1OC(=O)CCC(OCCCCCCCC)OCCCCCCCC] | CZ-6 |
| 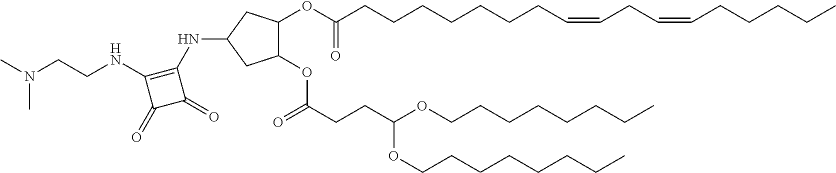 [SMILES: CCCCC/C=C\C/C=C\CCCCCCCC(=O)OC1CC(Nc2c(NCCN(C)C)c(=O)c2=O)CC1OC(=O)CCC(OCCCCCCCC)OCCCCCCCC] | CZ-7 |
| 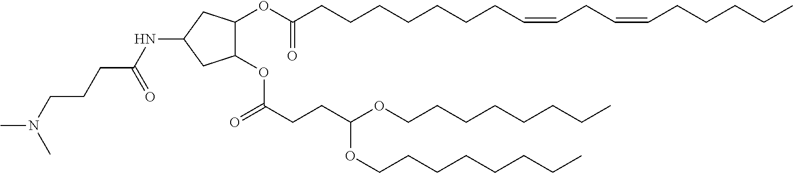 [SMILES: CCCCC/C=C\C/C=C\CCCCCCCC(=O)OC1CC(NC(=O)CCCN(C)C)CC1OC(=O)CCC(OCCCCCCCC)OCCCCCCCC] | CZ-8 |
| 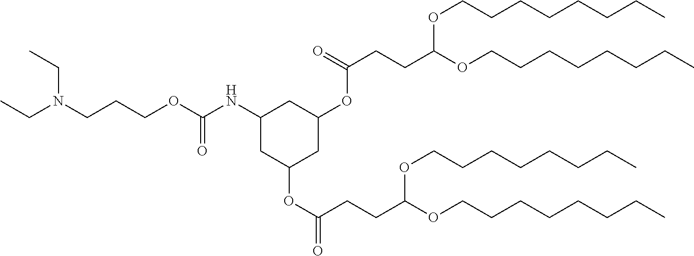 [SMILES: CCCCCCCCOC(CCC(=O)OC1CC(NC(=O)OCCCN(CC)CC)CC(OC(=O)CCC(OCCCCCCCC)OCCCCCCCC)C1)OCCCCCCCC] | CZ-9 |
| 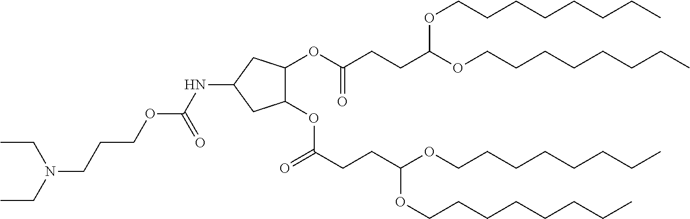 [SMILES: CCCCCCCCOC(CCC(=O)OC1CC(NC(=O)OCCCN(CC)CC)CC1OC(=O)CCC(OCCCCCCCC)OCCCCCCCC)OCCCCCCCC] | CZ-10 |
| 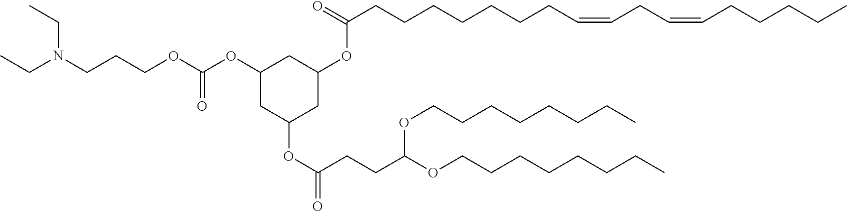 [SMILES: CCCCC/C=C\C/C=C\CCCCCCCC(=O)OC1CC(OC(=O)CCC(OCCCCCCCC)OCCCCCCCC)CC(OC(=O)OCCCN(CC)CC)C1] | CZ-11 |
| 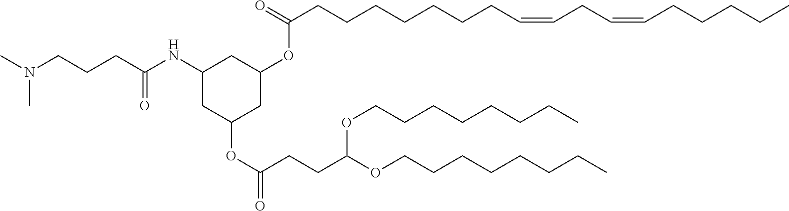 [SMILES: CCCCC/C=C\C/C=C\CCCCCCCC(=O)OC1CC(NC(=O)CCCN(C)C)CC(OC(=O)CCC(OCCCCCCCC)OCCCCCCCC)C1] | CZ-12 |
| 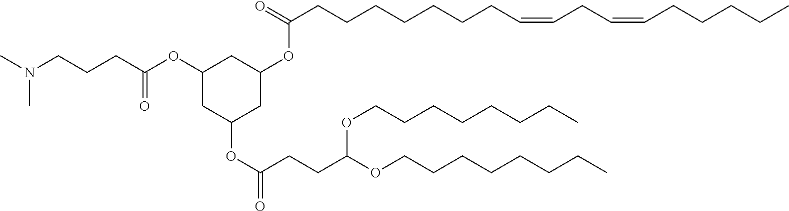 [SMILES: CCCCC/C=C\C/C=C\CCCCCCCC(=O)OC1CC(OC(=O)CCCN(C)C)CC(OC(=O)CCC(OCCCCCCCC)OCCCCCCCC)C1] | CZ-13 |
| 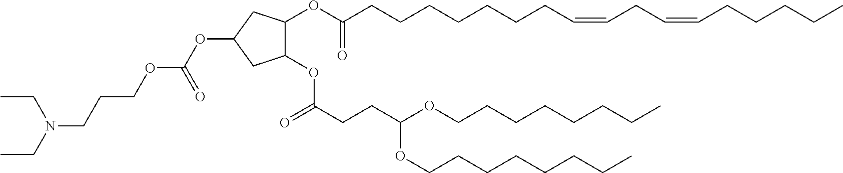 [SMILES: CCCCC/C=C\C/C=C\CCCCCCCC(=O)OC1CC(OC(=O)OCCCN(CC)CC)CC1OC(=O)CCC(OCCCCCCCC)OCCCCCCCC] | CZ-14 |
| 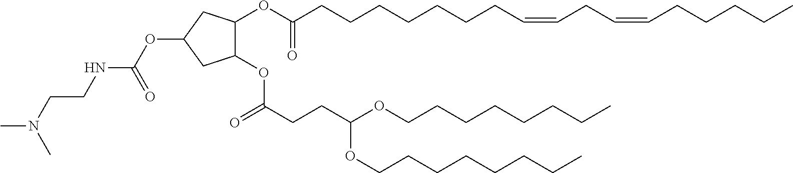 [SMILES: CCCCC/C=C\C/C=C\CCCCCCCC(=O)OC1CC(OC(=O)NCCN(C)C)CC1OC(=O)CCC(OCCCCCCCC)OCCCCCCCC] | CZ-15 |
| 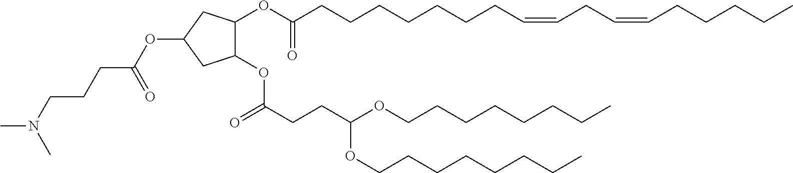 [SMILES: CCCCC/C=C\C/C=C\CCCCCCCC(=O)OC1CC(OC(=O)CCCN(C)C)CC1OC(=O)CCC(OCCCCCCCC)OCCCCCCCC] | CZ-16 |
| 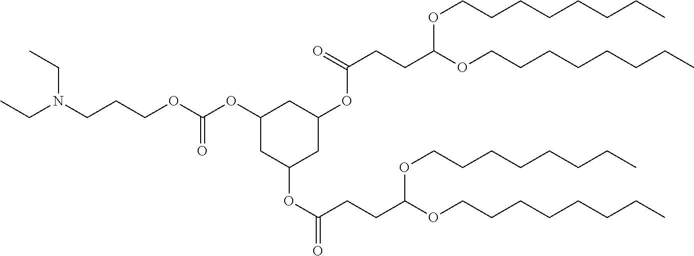 [SMILES: CCCCCCCCOC(CCC(=O)OC1CC(OC(=O)CCC(OCCCCCCCC)OCCCCCCCC)CC(OC(=O)OCCCN(CC)CC)C1)OCCCCCCCC] | CZ-17 |
| 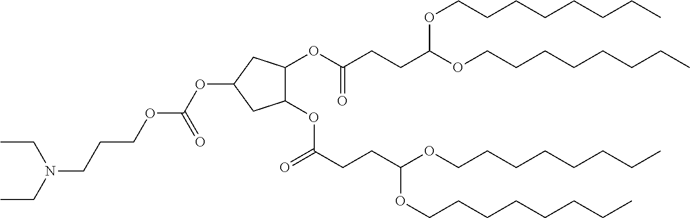 [SMILES: CCCCCCCCOC(CCC(=O)OC1CC(OC(=O)OCCCN(CC)CC)CC1OC(=O)CCC(OCCCCCCCC)OCCCCCCCC)OCCCCCCCC] | CZ-18 |

ii. Structural Lipids

In some embodiments, an LNP comprises a structural lipid. Structural lipids can be selected from the group consisting of, but are not limited to, cholesterol, fecosterol, fucosterol, beta sitosterol, sitosterol, ergosterol, campesterol, stigmasterol, brassicasterol, tomatidine, cholic acid, sitostanol, litocholic acid, tomatine, ursolic acid, alpha-tocopherol, Vitamin D3, Vitamin D2, Calcipotriol, botulin, lupeol, oleanolic acid, beta-sitosterol-acetate and mixtures thereof. In some embodiments, the structural lipid is cholesterol. In some embodiments, the structural lipid is a cholesterol analogue disclosed by Patel, et al., Nat Commun., 11, 983 (2020), which is incorporated herein by reference in its entirety. In some embodiments, the structural lipid includes cholesterol and a corticosteroid (such as prednisolone, dexamethasone, prednisone, and hydrocortisone), or any combinations thereof. In some embodiments, a structural lipid is described in international patent application WO2019152557A1, which is incorporated herein by reference in its entirety.

In some embodiments, a structural lipid is a cholesterol analog. Using a cholesterol analog may enhance endosomal escape as described in Patel et al., Naturally-occurring cholesterol analogues in lipid nanoparticles induce polymorphic shape and enhance intracellular delivery of mRNA, Nature Communications (2020), which is incorporated herein by reference.

In some embodiments, a structural lipid is a phytosterol. Using a phytosterol may enhance endosomal escape as described in Herrera et al., Illuminating endosomal escape of polymorphic lipid nanoparticles that boost mRNA delivery, Biomaterials Science (2020), which is incorporated herein by reference.

In some embodiments, a structural lipid contains plant sterol mimetics for enhanced endosomal release.

iii. PEGylated Lipids

A PEGylated lipid is a lipid modified with polyethylene glycol.

In some embodiments, an LNP comprises one, two or more PEGylated lipid or PEG-modified lipid. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, the PEGylated lipid is selected from (R)-2,3-bis(octadecyloxy)propyl-1-(methoxypoly(ethyleneglycol)2000)propylcarbamate, PEG-S-DSG, PEG-S-DMG, PEG-PE, PEG-PAA, PEG-OH DSPE C18, PEG-DSPE, PEG-DSG, PEG-DPG, PEG-DOMG, PEG-DMPE Na, PEG-DMPE, PEG-DMG2000, PEG-DMG C14, PEG-DMG 2000, PEG-DMG, PEG-DMA, PEG-Ceramide C16, PEG-C-DOMG, PEG-c-DMOG, PEG-c-DMA, PEG-cDMA, PEGA, PEG750-C-DMA, PEG400, PEG2k-DMG, PEG2k-C11, PEG2000-PE, PEG2000P, PEG2000-DSPE, PEG2000-DOMG, PEG2000-DMG, PEG2000-C-DMA, PEG2000, PEG200, PEG(2k)-DMG, PEG DSPE C18, PEG DMPE C14, PEG DLPE C12, PEG Click DMG C14, PEG Click C12, PEG Click C10, N(Carbonyl-methoxypolyethylenglycol-2000)-1,2-distearoyl-sn-glycero3-phosphoethanolamine, Myrj52, mPEG-PLA, MPEG-DSPE, mPEG3000-DMPE, MPEG-2000-DSPE, MPEG2000-DSPE, mPEG2000-DPPE, mPEG2000-DMPE, mPEG2000-DMG, mDPPE-PEG2000, 1,2-distearoyl-sn-glycero-3-phosphoethanolamine-PEG2000, HPEG-2K-LIPD, Folate PEG-DSPE, DSPE-PEGMA 500, DSPE-PEGMA, DSPE-PEG6000, DSPE-PEG5000, DSPE-PEG2K-NAG, DSPE-PEG2k, DSPE-PEG2000maleimide, DSPE-PEG2000, DSPE-PEG, DSG-PEGMA, DSG-PEG5000, DPPE-PEG-2K, DPPE-PEG, DPPE-mPEG2000, DPPE-mPEG, DPG-PEGMA, DOPE-PEG2000, DMPE-PEGMA, DMPE-PEG2000, DMPE-Peg, DMPE-mPEG2000, DMG-PEGMA, DMG-PEG2000, DMG-PEG, distearoyl-glycerol-polyethyleneglycol, C18PEG750, C18PEG5000, C18PEG3000, C18PEG2000, C16PEG2000, C14PEG2000, C18-PEG5000, C18PEG, C16PEG, C16 mPEG (polyethylene glycol) 2000 Ceramide, C14-PEG-DSPE200, C14-PEG2000, C14PEG2000, C14-PEG 2000, C14-PEG, C14PEG, 14.0-PEG2KPE, 1,2-distearoyl-sn-glycero-3-phosphoethanolamine-PEG2000, (R)-2,3-bis(octadecyloxy)propyl-1-(methoxypoly(ethyleneglycol)2000)propylcarbamate, (PEG)-C-DOMG, PEG-C-DMA, and DSPE-PEG-X.

In some embodiments, the LNP comprises a PEGylated lipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2015/0203446; US 2017/0210697; US 2014/0200257; or WO 2019/089828A1, each of which is incorporated by reference herein in their entirety.

In some embodiments, the LNP comprises a PEGylated lipid substitute in place of the PEGylated lipid. All embodiments disclosed herein that contemplate a PEGylated lipid should be understood to also apply to PEGylated lipid substitutes. In some embodiments, the LNP comprises a polysarcosine-lipid conjugate, such as those disclosed in US 2022/0001025 A1, which is incorporated by reference herein in its entirety.

iv. Phospholipids

In some embodiments, an LNP of the present disclosure comprises a phospholipid. Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sodium (S)-2-ammonio-3-((((R)-2-(oleoyloxy)-3-(stearoyloxy)propoxy)oxidophosphoryl)oxy)propanoate (L-α-phosphatidylserine; Brain PS), dimyristoyl phosphatidylcholine (DMPC), dimyristoyl phosphoethanolamine (DMPE), dimyristoylphosphatidylglycerol (DMPG), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), 1,2-Dielaidoyl-sn-phosphatidylethanolamine (DEPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), 1,2-dioleoyl-sn-glycero-3-phosphate (18:1 PA; DOPA), ammonium bis((S)-2-hydroxy-3-(oleoyloxy)propyl) phosphate (18:1 DMP; LBPA), 1,2-dioleoyl-sn-glycero-3-phospho-(1′-myo-inositol) (DOPI; 18:1 PI), 1,2-distearoyl-sn-glycero-3-phospho-L-serine (18:0 PS), 1,2-dilinoleoyl-sn-glycero-3-phospho-L-serine (18:2 PS), 1-palmitoyl-2-oleoyl-sn-glycero-3-phospho-L-serine (16:0-18:1 PS; POPS), 1-stearoyl-2-oleoyl-sn-glycero-3-phospho-L-serine (18:0-18:1 PS), 1-stearoyl-2-linoleoyl-sn-glycero-3-phospho-L-serine (18:0-18:2 PS), 1-oleoyl-2-hydroxy-sn-glycero-3-phospho-L-serine (18:1 Lyso PS), 1-stearoyl-2-hydroxy-sn-glycero-3-phospho-L-serine (18:0 Lyso PS), and sphingomyelin. In some embodiments, an LNP includes DSPC. In certain embodiments, an LNP includes DOPE. In some embodiments, an LNP includes both DSPC and DOPE.

In some embodiments, an LNP comprises a phospholipid selected from 1-pentadecanoyl-2-oleoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-palmitoyl-2-oleoyl-glycero-3-phosphocholine, 1-palmitoyl-2-linoleoyl-sn-glycero-3-phosphocholine, 1-palmitoyl-2-arachidonoyl-sn-glycero-3-phosphocholine, 1-palmitoyl-2-docosahexaenoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-stearoyl-2-oleoyl-sn-glycero-3-phosphocholine, 1-stearoyl-2-linoleoyl-sn-glycero-3-phosphocholine, 1-stearoyl-2-arachidonoyl-sn-glycero-3-phosphocholine, 1-stearoyl-2-docosahexaenoyl-sn-glycero-3-phosphocholine, 1-oleoyl-2-myristoyl-sn-glycero-3-phosphocholine, 1-oleoyl-2-palmitoyl-sn-glycero-3-phosphocholine, 1-oleoyl-2-stearoyl-sn-glycero-3-phosphocholine, 1-palmitoyl-2-acetyl-sn-glycero-3-phosphocholine, 1,2-dioleoyl-sn-glycero-3-phospho-(1′-myo-inositol-3′,4′-bisphosphate), 1,2-dioleoyl-sn-glycero-3-phospho-(1′-myo-inositol-3′,5′-bisphosphate), 1,2-dioleoyl-sn-glycero-3-phospho-(1′-myo-inositol-4′,5′-bisphosphate), 1,2-dioleoyl-sn-glycero-3-phospho-(1′-myo-inositol-3′,4′,5′-trisphosphate), 1,2-dioleoyl-sn-glycero-3-phospho-(1′-myo-inositol-3′-phosphate), 1,2-dioleoyl-sn-glycero-3-phospho-(1′-myo-inositol-4′-phosphate), 1,2-dioleoyl-sn-glycero-3-phospho-(1′-myo-inositol-5′-phosphate), 1,2-dioleoyl-sn-glycero-3-phospho-(1′-myo-inositol), 1,2-dioleoyl-sn-glycero-3-phospho-L-serine, and 1-(8Z-octadecenoyl)-2-palmitoyl-sn-glycero-3-phosphocholine.

In some embodiments, a phospholipid tail may be modified in order to promote endosomal escape as described in U.S. Application Publication 2021/0121411, which is incorporated herein by reference.

In some embodiments, the LNP comprises a phospholipid disclosed in one of US 2019/0240354; US 2010/0130588; US 2021/0087135; WO 2021/204179; US 2021/0128488; US 2020/0121809; US 2017/0119904; US 2013/0108685; US 2013/0195920; US 2015/0005363; US 2014/0308304; US 2013/0053572; WO 2019/232095A1; WO 2021/077067; WO 2019/152557; US 2017/0210697; or WO 2019/089828A1, each of which is incorporated by reference herein in their entirety.

In some embodiments, phospholipids disclosed in US 2020/0121809 have the following structure:

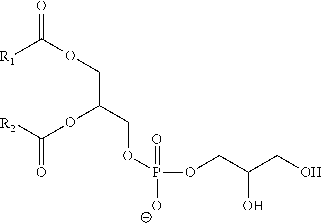

wherein R¹ and R² are each independently a branched or straight, saturated or unsaturated carbon chain (e.g., alkyl, alkenyl, alkynyl).
vi. Targeting Moieties

In some embodiments, the lipid nanoparticle further comprises a targeting moiety. The targeting moiety may be an antibody or a fragment thereof. The targeting moiety may be capable of binding to a target antigen.

In some embodiments, the pharmaceutical composition comprises a targeting moiety that is operably connected to a lipid nanoparticle. In some embodiments, the targeting moiety is capable of binding to a target antigen. In some embodiments, the target antigen is expressed in a target organ. In some embodiments, the target antigen is expressed more in the target organ than it is in the liver.

In some embodiments, the targeting moiety is an antibody as described in WO2016189532A1, which is incorporated herein by reference. For example, in some embodiments, the targeted particles are conjugated to a specific anti-CD38 monoclonal antibody (mAb), which allows specific delivery of the siRNAs encapsulated within the particles at a greater percentage to B-cell lymphocytes malignancies (such as MCL) than to other subtypes of leukocytes.

In some embodiments, the lipid nanoparticles may be targeted when conjugated/attached/associated with a targeting moiety such as an antibody.

vii. Zwitterionic Amino Lipids

In some embodiments, an LNP comprises a zwitterionic lipid. In some embodiments, an LNP comprising a zwitterionic lipid does not comprise a phospholipid.

Zwitterionic amino lipids have been shown to be able to self-assemble into LNPs without phospholipids to load, stabilize, and release mRNAs intracellularly as described in U.S. Patent Application 20210121411, which is incorporated herein by reference in its entirety. Zwitterionic, ionizable cationic and permanently cationic helper lipids enable tissue-selective mRNA delivery and CRISPR-Cas9 gene editing in spleen, liver and lungs as described in Liu et al., Membrane-destabilizing ionizable phospholipids for organ-selective mRNA delivery and CRISPR-Cas gene editing, Nat Mater. (2021), which is incorporated herein by reference in its entirety.

The zwitterionic lipids may have head groups containing a cationic amine and an anionic carboxylate as described in Walsh et al., Synthesis, Characterization and Evaluation of Ionizable Lysine-Based Lipids for siRNA Delivery, Bioconjug Chem. (2013), which is incorporated herein by reference in its entirety. Ionizable lysine-based lipids containing a lysine head group linked to a long-chain dialkylamine through an amide linkage at the lysine α-amine may reduce immunogenicity as described in Walsh et al., Synthesis, Characterization and Evaluation of Ionizable Lysine-Based Lipids for siRNA Delivery, Bioconjug Chem. (2013).

viii. Additional Lipid Components

In some embodiments, the LNP compositions of the present disclosure further comprise one or more additional lipid components capable of influencing the tropism of the LNP. In some embodiments, the LNP further comprises at least one lipid selected from DDAB, EPC, 14PA, 18BMP, DODAP, DOTAP, and C12-200 (see Cheng, et al. Nat Nanotechnol. 2020 April; 15(4): 313-320.; Dillard, et al. PNAS 2021 Vol. 118 No. 52.).

In some embodiments, the LNP compositions of the present disclosure comprise, or further comprise one or more lipids selected from 1,2-di-O-octadecenyl-sn-glycero-3-phosphocholine (18:0 Diether PC), 1,2-dilinolenoyl-sn-glycero-3-phosphocholine (18:3 PC), Acylcarnosine (AC), 1-hexadecyl-sn-glycero-3-phosphocholine (C16 Lyso PC), N-oleoyl-sphingomyelin (SPM) (C18:1), N-lignoceryl SPM (C24:0), N-nervonoylshphingomyelin (C24:1), Cardiolipin (CL), 1,2-bis(tricosa-10,12-diynoyl)-sn-glycero-3-phosphocholine (DC8-9PC), dicetyl phosphate (DCP), dihexadecyl phosphate (DCP1), 1,2-Dipalmitoylglycerol-3-hemisuccinate (DGSucc), short-chain bis-n-heptadecanoyl phosphatidylcholine (DHPC), dihexadecoyl-phosphoethanolamine (DHPE), 1,2-dilinoleoyl-sn-glycero-3-phosphocholine (DLPC), 1,2-dilauroyl-sn-glycero-3-PE (DLPE), dimyristoyl glycerol hemisuccinate (DMGS), dimyristoyl phosphatidylcholine (DMPC), dimyristoyl phosphoethanolamine (DMPE), dimyristoylphosphatidylglycerol (DMPG), dioleyloxybenzylalcohol (DOBA), 1,2-dioleoylglyceryl-3-hemisuccinate (DOGHEMS), N-[2-(2-{2-[2-(2,3-Bis-octadec-9-enyloxy-propoxy)-ethoxy]-ethoxy}-ethoxy)-ethyl]-3-(3,4,5-1rihydroxy-6-hydroxymethyl-1etrahydro-pyran-2-ylsulfanyl)-propionamide (DOGP4αMan), dioleoylphosphatidylcholine (DOPC), dioleoylphosphatidylethanolamine (DOPE), dioleoyl-phosphatidylethanolamine4-(N-maleimidomethyl)-cyclohexane-1-carboxylate (DOPE-mal), dioleoylphosphatidylglycerol (DOPG), 1,2-dioleoyl-sn-glycero-3-(phospho-L-serine) (DOPS), acell-fusogenicphospholipid (DPhPE), dipalmitoylphosphatidylethanolamine (DPPE), dipalmitoylphosphatidylglycerol (DPPG), dipalmitoylphosphatidylserine (DPPS), distearoylphosphatidylcholine (DSPC), distearoyl-phosphatidyl-ethanolamine (DSPE), distearoyl phosphoethanolamineimidazole (DSPEI), 1,2-diundecanoyl-sn-glycero-phosphocholine (DUPC), egg phosphatidylcholine (EPC), histaminedistearoylglycerol (HDSG), 1,2-Dipalmitoylglycerol-hemisuccinate-Nα-Histidinyl-Hemisuccinate (HistSuccDG), N-(5′-hydroxy-3′-oxypentyl)-10-12-pentacosadiynamide (h-Pegi-PCDA), 2-[1-hexyloxyethyl]-2-devinylpyropheophorbide-a (HPPH), hydrogenatedsoybeanphosphatidylcholine (HSPC), 1,2-Dipalmitoylglycerol-O-α-histidinyl-Na-hemisuccinate (IsohistsuccDG), mannosialized dipalmitoylphosphatidylethanolamine (ManDOG), 1,2-Dioleoyl-sn-Glycero-3-Phosphoethanolamine-N-[4-(p-maleimidomethyl)cyclohexane-carboxamide](MCC-PE), 1,2-diphytanoyl-sn-glycero-3-phosphoethanolamine (ME 16:0 PE), 1-myristoyl-2-hydroxy-sn-glycero-phosphocholine (MHPC), a thiol-reactive maleimide headgroup lipid e.g. 1,2-dioleoyl-sn-glycero-3-phosphoethanolamine-N-[4-(p-maleimidophenyl)but-yramid (MPB-PE), Nervonic Acid (NA), sodium cholate (NaChol), 1,2-dioleoyl-sn-glycero-3-[phosphoethanolamine-N-dodecanoyl (NC12-DOPE), 1-oleoyl-2-cholesteryl hemisuccinoyl-sn-glycero-3-phosphocholine (OChemsPC), phosphatidylethanolamine lipid (PE), PE lipid conjugated with polyethylene glycol (PEG) (e.g., polyethylene glycol-distearoylphosphatidylethanolamine lipid (PEG-PE)), phosphatidylglycerol (PG), partially hydrogenated soy phosphatidylchloline (PHSPC), phosphatidylinositol lipid (PI), phosphotidylinositol-4-phosphate (PIP), palmitoyloleoylphosphatidylcholine (POPC), phosphatidylethanolamine (POPE), palmitoyloleyolphosphatidylglycerol (POPG), phosphatidylserine (PS), lissamine rhodamine B-phosphatidylethanolamine lipid (Rh-PE), purified soy-derived mixture of phospholipids (SIOO), phosphatidylcholine (SM), 18-1-trans-PE,1-stearoyl-2-oleoyl-phosphatidyethanolamine (SOPE), soybean phosphatidylcholine (SPC), sphingomyelins (SPM), alpha,alpha-trehalose-6,6′-dibehenate (TDB), 1,2-dielaidoyl-sn-glycero-3-phophoethanolamine (transDOPE), ((23S,5R)-3-(bis(hexadecyloxy)methoxy)-5-(5-methyl-2,4-dioxo-3,4-dihydropyrimidin-1(2H)-yl)tetrahydrofuran-2-yl)methylmethylphosphate, 1,2-diarachidonoyl-sn-glycero-3-phosphocholine, 1,2-diarachidonoyl-sn-glycero-3-phosphoethanolamine, 1,2-didocosahexaenoyl-sn-glycero-3-phosphocholine, 1,2-didocosahexaenoyl-sn-glycero-3-phosphoethanolamine, 1,2-dilinolenoyl-sn-glycero-3-phosphocholine, 1,2-dilinolenoyl-sn-glycero-3-phosphoethanolamine, 1,2-dilinoleoyl-sn-glycero-3-phosphoethanolamine, 1,2-dioleyl-sn-glycero-3-phosphoethanolamine, 1,2-distearoyl-sn-glycero-3-phosphoethanolamine, 16-O-monomethyl PE, 16-O-dimethyl PE, and dioleylphosphatidylethanolamine.

viii. LNP Pharmaceutical Compositions

In some embodiments, a nanoparticle includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 18.5 mol % to about 48.5 mol % structural lipid, and about 0 mol % to about 10 mol % of PEG lipid, provided that the total mol % does not exceed 100%. In some embodiments, the lipid component of the nanoparticle composition includes about 35 mol % to about 55 mol % ionizable lipid, about 5 mol % to about 25 mol % phospholipid, about 30 mol % to about 40 mol % structural lipid, and about 0 mol % to about 10 mol % of PEG lipid. In a particular embodiment, the lipid component includes about 50 mol % ionizable lipid, about 10 mol % phospholipid, about 38.5 mol % structural lipid, and about 1.5 mol % of PEG lipid. In another particular embodiment, the lipid component includes about 40 mol % ionizable lipid, about 20 mol % phospholipid, about 38.5 mol % structural lipid, and about 1.5 mol % of PEG lipid. In another particular embodiment, the lipid component includes about 48.5 mol % ionizable lipid, about 10 mol % phospholipid, about 40 mol % structural lipid, and about 1.5 mol % of PEG lipid. In another particular embodiment, the lipid component includes about 48.5 mol % ionizable lipid, about 10 mol % phospholipid, about 39 mol % structural lipid, and about 2.5 mol % of PEG lipid. In some embodiments, the phospholipid may be DOPE or DSPC. In other embodiments, the PEG lipid may be PEG-DMG and/or the structural lipid may be cholesterol. The amount of active agent in a nanoparticle composition may depend on the size, composition, desired target and/or application, or other properties of the nanoparticle composition as well as on the properties of the active agent. For example, the amount of active agent useful in a nanoparticle composition may depend on the size, sequence, and other characteristics of the active agent. The relative amounts of active agent and other elements (e.g., lipids) in a nanoparticle composition may also vary. In some embodiments, the wt/wt ratio of the lipid component to an enzyme in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. The amount of a enzyme in a nanoparticle composition may, for example, be measured using absorption spectroscopy (e.g., ultraviolet-visible spectroscopy).

In some embodiments, a nanoparticle composition comprising an active agent of the present disclosure is formulated to provide a specific E:P ratio. The E:P ratio of the composition refers to the molar ratio of nitrogen atoms in one or more lipids to the number of phosphate groups in an RNA active agent. In general, a lower E:P ratio is preferred. The one or more enzymes, lipids, and amounts thereof may be selected to provide an E:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the E:P ratio may be from about 2:1 to about 8:1. In other embodiments, the E:P ratio is from about 5:1 to about 8:1. For example, the E:P ratio may be about 5.0:1, about 5.5:1, about 5.67:1, about 6.0:1, about 6.5:1, or about 7.0:1.

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition. Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure Zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, Such as particle size, polydispersity index, and Zeta potential.

The mean size of a nanoparticle composition may be between 10s of nm and 100s of nm, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25.

The Zeta potential of a nanoparticle composition may be used to indicate the electrokinetic potential of the composition. For example, the Zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the Zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV, to about +20 mV, from about 0 mV to about +15 mV, from about 0 mV to about +10 mV, from about 0 mV to about +5 mV, from about +5 mV to about +20 mV, from about +5 mV, to about +15 mV, or from about +5 mV to about +10 mV.

The efficiency of encapsulation of a payload describes the amount of payload that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of payload in a solution containing the nanoparticle composition before and after breaking up the nanoparticle composition with one or more organic solvents or detergents. Fluorescence may be used to measure the amount of free payload in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

Lipids and their method of preparation are disclosed in, e.g., U.S. Pat. Nos. 8,569,256, 5,965,542 and U.S. Patent Publication Nos. 2016/0199485, 2016/0009637, 2015/0273068, 2015/0265708, 2015/0203446, 2015/0005363, 2014/0308304, 2014/0200257, 2013/086373, 2013/0338210, 2013/0323269, 2013/0245107, 2013/0195920, 2013/0123338, 2013/0022649, 2013/0017223, 2012/0295832, 2012/0183581, 2012/0172411, 2012/0027803, 2012/0058188, 2011/0311583, 2011/0311582, 2011/0262527, 2011/0216622, 2011/0117125, 2011/0091525, 2011/0076335, 2011/0060032, 2010/0130588, 2007/0042031, 2006/0240093, 2006/0083780, 2006/0008910, 2005/0175682, 2005/017054, 2005/0118253, 2005/0064595, 2004/0142025, 2007/0042031, 1999/009076 and PCT Pub. Nos. WO 99/39741, WO 2017/117528, WO 2017/004143, WO 2017/075531, WO 2015/199952, WO 2014/008334, WO 2013/086373, WO 2013/086322, WO 2013/016058, WO 2013/086373, WO2011/141705, and WO 2001/07548 and Semple et. al, Nature Biotechnology, 2010, 28, 172-176, the full disclosures of which are herein incorporated by reference in their entirety for all purposes.

A nanoparticle composition may include any substance useful in pharmaceutical compositions. For example, the nanoparticle composition may include one or more pharmaceutically acceptable excipients or accessory ingredients such as, but not limited to, one or more solvents, dispersion media, diluents, dispersion aids, suspension aids, granulating aids, disintegrants, fillers, glidants, liquid vehicles, binders, surface active agents, isotonic agents, thickening or emulsifying agents, buffering agents, lubricating agents, oils, preservatives, and other species. Excipients such as waxes, butters, coloring agents, coating agents, flavorings, and perfuming agents may also be included. Pharmaceutically acceptable excipients are well known in the art (see for example Remington's The Science and Practice of Pharmacy, 21^{st} Edition, A. R. Gennaro: Lippincott, Williams & Wilkins, Baltimore, Md., 2006). Other different lipids or liposomal formulations including nanoparticles and methods of administration include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

In some embodiments, the LNP encapsulates the engineered retron, e.g., an engineered nucleic acid construct, ncRNA, vector system, RNA molecule, and/or engineered nucleic acid-enzyme construct as described herein.

In some embodiments, the lipid nanoparticle comprises: one or more ionizable lipids; one or more structural lipids; one or more PEGylated lipids; and one or more phospholipids. In some embodiments, the one or more ionizable lipids is selected from the group consisting of those disclosed in Table X.

In some embodiments, the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone. In some embodiments, the one or more PEGylated lipids are selected from the group consisting of PEG-c-DOMG, PEG-DMG, PEG-DLPE, PEG-DMPE, PEG-DPPC, and PEG-DSPE.

In some embodiments, the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

In some embodiments, the lipid nanoparticle comprises about 48.5 mol % ionizable lipid, about 10 mol % phospholipid, about 40 mol % structural lipid, and about 1.5 mol % of PEG lipid.

In some embodiments, the lipid nanoparticle comprises about 48.5 mol % ionizable lipid, about 10 mol % phospholipid, about 39 mol % structural lipid, and about 2.5 mol % of PEG lipid. In some embodiments, the LNP further comprises a targeting moiety operably connected to the LNP. In some embodiments, the LNP further comprises one or more additional components selected from the group consisting of DDAB, EPC, 14PA, 18BMP, DODAP, DOTAP, and C12-200.

In some embodiments, the engineered retron can be used for gene transfer, which may be performed under ex vivo or in vivo conditions. Ex vivo gene therapy refers to the isolation of cells from a subject, the delivery of a nucleic acid into cells in vitro, and the return of the modified cells back into the subject. This may involve the collection of a biological sample comprising cells from the subject. For example, blood can be obtained by venipuncture, and solid tissue samples can be obtained by surgical techniques according to methods well known in the art.

Usually, but not always, the subject who receives the cells (e.g., the recipient) is also the subject from whom the cells are harvested or obtained, which provides the advantage that the donated cells are autologous. However, cells can be obtained from another subject (e.g., a donor), a culture of cells from a donor, or from established cell culture lines. Accordingly, in some embodiments the cells are allogeneic to the recipient. Cells may be obtained from the same or a different species than the subject to be treated, but preferably are of the same species, and more preferably of the same immunological profile as the subject. Such cells can be obtained, for example, from a biological sample comprising cells from a close relative or matched donor, then transfected with nucleic acids (e.g., comprising an engineered retron), and administered to a subject in need of genome modification, for example, for treatment of a disease or condition.

In other embodiments, the engineered retron can be introduced in vivo (e.g., used in gene therapy) by physically delivering the engineered retron to a subject. Examples of physically introducing the engineered retron includes via injections, electroporation and transfection (e.g., calcium-mediated or liposome transfection, or the like).

### J. PAYLOADS

The retron-based gene editing systems and/or components thereof may be delivered by way of LNPs as described here. In various embodiments, the retron-based gene editing systems may be delivered by LNPs into cells, tissues, organs, or organisms. Depending on the chosen format, the retron-based gene editing systems and/or the individual or combined components thereof may be delivered as DNA molecules (e.g., encoded on one or more plasmids), RNA molecules (e.g., guide RNAs for targeting a programmable nuclease or linear or circular mRNAs coding for the retron RTs or programmable nuclease components of the retron-based gene editing systems), proteins (e.g., retron polypeptides, accessory proteins having other functions (e.g., recombinases, nucleases, polymerases, ligases, deaminases, or reverse transcriptases), or protein-nucleic acid complexes (e.g., complexes between a guide RNA and a programmable nuclease protein or fusion protein comprising a retron RT). These DNA, RNA, protein, or nucleoprotein corresponding to and/or encoding the retron-based gene editing systems or components thereof comprise the LNP cargo or payloads. In various embodiments, the LNP cargo or payloads may comprise nucleic acid payloads, including coding payloads such as linear and circular mRNA for encoding the various components of the retron-based editing system.

### 1. Nucleic Acid Payloads

In various embodiments, the LNP compositions described herein can be used to deliver a nucleic acid or polynucleotide payload, e.g., a linear or circular mRNA.

In various embodiments, the retron-based editing compositions described herein can include a nucleic acid or polynucleotide payload, e.g., a linear or circular mRNA. For example, the retron gene editing systems may comprise one or more coding mRNA (circular or linear) for encoding retron RT and other accessory proteins (e.g., programmable nuclease) and these RNA components may be delivered by LNPs.

In some embodiments, a LNP is capable of delivering a polynucleotide to a target cell, tissue, or organ. A polynucleotide, in its broadest sense of the term, includes any compound and/or substance that is or can be incorporated into an oligonucleotide chain. Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc. RNAs useful in the compositions and methods described herein can be selected from the group consisting of but are not limited to, shortimers, antagomirs, antisense, ribozymes, short interfering RNA (siRNA), asymmetrical interfering RNA (aiRNA), microRNA (miRNA), Dicer substrate RNA (dsRNA), short hairpin RNA (shRNA), transfer RNA (tRNA), messenger RNA (mRNA), and mixtures thereof. In some embodiments, a polynucleotide is mRNA. In some embodiments, a polynucleotide is circular RNA. In some embodiments, a polynucleotide encodes a protein, e.g., a nucleobase editing enzyme. A polynucleotide may encode any polypeptide of interest, including any naturally or non-naturally occurring or otherwise modified polypeptide. A polypeptide may be of any size and may have any secondary structure or activity. In some embodiments, a polypeptide encoded by an mRNA may have a therapeutic effect when expressed in a cell.

In other embodiments, a polynucleotide is an siRNA. An siRNA may be capable of selectively knocking down or down regulating expression of a gene of interest. For example, an siRNA could be selected to silence a gene associated with a particular disease, disorder, or condition upon administration to a subject in need thereof of a nanoparticle composition including the siRNA. An siRNA may comprise a sequence that is complementary to an mRNA sequence that encodes a gene or protein of interest. In some embodiments, the siRNA may be an immunomodulatory siRNA.

In some embodiments, a polynucleotide is an shRNA or a vector or plasmid encoding the same. An shRNA may be produced inside a target cell upon delivery of an appropriate construct to the nucleus. Constructs and mechanisms relating to shRNA are well known in the relevant arts.

A polynucleotide may include a first region of linked nucleosides encoding a polypeptide of interest (e.g., a coding region), a first flanking region located at the 5′-terminus of the first region (e.g., a 5′-UTR), a second flanking region located at the 3-terminus of the first region (e.g., a 3′-UTR), at least one 5′-cap region, and a 3-stabilizing region. In some embodiments, a polynucleotide further includes a poly-A region or a Kozak sequence (e.g., in the 5′-UTR). In some cases, polynucleotides may contain one or more intronic nucleotide sequences capable of being excised from the polynucleotide. In some embodiments, a polynucleotide (e.g., an mRNA) may include a 5′cap structure, a chain terminating nucleotide, a stem loop, a polyA sequence, and/or a polyadenylation signal. Any one of the regions of a nucleic acid may include one or more alternative components (e.g., an alternative nucleoside). For example, the 3′-stabilizing region may contain an alternative nucleoside such as an L-nucleoside, an inverted thymidine, or a 2′-O-methyl nucleoside and/or the coding region, 5′-UTR, 3′-UTR, or cap region may include an alternative nucleoside such as a 5-substituted uridine (e.g., 5-methoxyuridine), a 1-substituted pseudouridine (e.g., 1-methyl pseudouridine or 1-ethyl-pseudouridine), and/or a 5-substituted cytidine (e.g., 5-methyl-cytidine). In some embodiments, a polynucleotide contains only naturally occurring nucleosides.

In some cases, a polynucleotide is greater than 30 nucleotides in length. In another embodiment, the poly nucleotide molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 50 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

In some embodiments, a polynucleotide molecule, formula, composition or method associated therewith comprises one or more polynucleotides comprising features as described in WO2002/098443, WO2003/051401, WO2008/052770, WO2009/127230, WO2006/122828, WO2008/083949, WO2010/088927, WO2010/037539, WO2004/004743, WO2005/016376, WO2006/024518, WO2007/095976, WO2008/014979, WO2008/077592, WO2009/030481, WO2009/095226, WO2011/069586, WO2011/026641, WO2011/144358, WO2012/019780, WO2012/013326, WO2012/089338, WO2012/113513, WO2012/116811, WO2012/116810, WO2013/113502, WO2013/113501, WO2013/113736, WO2013/143698, WO2013/143699, WO2013/143700, WO2013/120626, WO2013/120627, WO2013/120628, WO2013/120629, WO2013/174409, WO2014/127917, WO2015/024669, WO2015/024668, WO2015/024667, WO2015/024665, WO2015/024666, WO2015/024664, WO2015/101415, WO2015/101414, WO2015/024667, WO2015/062738, WO2015/101416, all of which are incorporated by reference herein.

In some embodiments, a polynucleotide comprises one or more microRNA binding sites. In some embodiments, a microRNA binding site is recognized by a microRNA in a non-target organ. In some embodiments, a microRNA binding site is recognized by a microRNA in the liver. In some embodiments, a microRNA binding site is recognized by a microRNA in hepatic cells.

In certain embodiments, an RNA of the present disclosure comprises one or more phosphonate modifications selected from a phosphorothioate linkage (PS), phosphorodithioate linkage (PS2), methylphosphonate linkage (MP), methoxypropylphosphonate linkage (MOP), 5′-(E)-vinylphosphonate linkage (5′-(E)-VP), 5′-Methyl Phosphonate linkage (5′-MP), (S)-5′-C-methyl with phosphate linkage, 5′-phosphorothioate linkage (5′-PS), and a peptide nucleic acid linkage (PNA). In certain embodiments, an RNA of the present disclosure comprises one or more ribose modifications selected from a 2′-O-methyl (2′-OMe), 2′-O-methoxyethyl (2′-O-MOE), 2′-deoxy-2′-fluoro (2′-F), 2′-arabino-fluoro (2′-Ara-F), 2′-O-benzyl, 2′-O-methyl-4-pyridine (2′-O-CH2Py(4)), Locked nucleic acid (LNA), (S)-cET-BNA, tricyclo-DNA (tcDNA), PMO, Unlocked Nucleic Acid (UNA) and glycol nucleic acid (GNA). In certain embodiments, the RNA comprises a Locked Nucleic Acid (LNA) comprising a methyl bridge, an ethyl bridge, a propyl bridge, a butyl bridge or an optionally substituted variant of any of the aforementioned. In certain embodiments, an RNA of the present disclosure comprises one or more modified bases selected from a pseudouridine (y), 2′thiouridine (s2U), N6′-methyladenosine (m⁶A), 5′methylcytidine (m⁵C), 5′fluoro2′-deoxyuridine, N-ethylpiperidine 7′-EAA triazole modified adenine, N-ethylpiperidine 6′triazole modified adenine, 6′pheynlpyrrolo-cytosine (PhpC), 2′,4′-difluorotoluyl ribonucleoside (rF), and 5′-nitroindole.

### 2. Single-Stranded DNA Payloads

In various embodiments, the LNPs of the present disclosure may comprise a payload having at least one single stranded DNA. In certain embodiments, the single stranded DNA is a linear single stranded DNA. In certain embodiments, the single stranded DNA is a circular single stranded DNA. In certain embodiments, the payload further comprises a nucleobase editing system, such as an enzyme or polynucleotide encoding an enzyme capable of independently or co-dependently editing, modifying, or altering a target polynucleotide sequence or a target transcript comprising a nucleic acid sequence.

In certain embodiments, the circular single stranded DNA (CiSSD) payload is one described in PCT Publication WO2020142730A1, which is incorporated by reference herein in its entirety. In certain embodiments, the CiSSD is a donor template for use as part of a nucleobase editing system for targeted genome modification. In certain embodiments, the CiSSD comprises a DNA insert, a 5′ homology arm, and a 3′ homology arm. In some embodiments, the DNA insert is located between the 5′ homology arm and the 3′ homology arm. Homology arms as used herein refer to a series of nucleotides that are complementary to a series of nucleotides in an endogenous DNA sequence in the target region. The homology arms flanking the DNA insert allow for specific insertion of the DNA insert in the target region. A target region is a nucleic acid sequence where a desired insertion or modification occurs.

In certain embodiments, the DNA insert is at least 1 nucleotide. In certain embodiments, the DNA insert is at least about 0.5 kb, 2 kb, 2.5 kb, 5 kb, 10 kb, 20 kb, 40 kb, 80 kb, 100 kb, 150 kb, or 200 kb. In certain embodiments, the length of the DNA insert is about 0.5 kb to 5 kb, about 1 kb to 5 kb, about 1 kb to 10 kb, about 1.6 kb to 5 kb, about 1.6 kb to 10 kb, about 2 kb to 5 kb, about 2 kb to 20 kb, about 2.5 kb to 5 kb, about 2.5 kb to 10 kb, about 2.5 kb to 20 kb, and about 5 kb to 100 kb. In some embodiments, the DNA insert size may range from about 1 kb to about 3 kb, about 3 kb to about 6 kb, about 6 kb to about 9 kb, about 9 kb to about 12 kb, about 12 kb to about 15 kb, about 15 kb to about 18 kb, or about 18 kb to about 21 kb.

In some embodiments, the DNA insert may comprise a nucleotide sequence that encodes a maker or a reporter, e.g., a fluorescent marker, an antibiotic marker, or any suitable marker. A “marker” or “reporter” as used herein means a feature that allows for identification and selection of a desired cell, e.g., by fluorescence or antibiotic resistance. For example, the insert may include a nucleotide sequence encoding a reporter (e.g, GFP, RFP, or any suitable reporter) or a recombinase. For example, the reporter is an N-terminal GFP fusion reporter.

In some embodiments, the DNA insert may comprise a nucleotide sequence that encodes a transcription unit, wherein each transcription unit can produce a cellular product (e.g, protein or RNA). In some embodiments, the DNA insert may comprise a nucleotide sequence that encodes a protein, e.g, an immunomodulatory protein (e.g, a cytokine), an antibody, a chimeric antigen receptor (CAR), a growth factor, a T cell receptor, or another protein.

In certain embodiments, the CiSSD comprises a DNA insert that can be inserted at a nucleotide break in a target region of genomic DNA. In some embodiments, the break is a double stranded break (DSB). In certain embodiments, the break is a single stranded DNA break or a nick. Precision gene editing techniques, e.g, CRISPR, create a break near a desired sequence change (target sequence). CRISPR can be applied to produce deletions, disruptions, insertions, replacements, and repairs. The components of template donors for these different modifications is generally the same, consisting of three basic elements: a 5′ homology arm, a DNA insert, and a 3′ homology arm. CRISPR-based gene editing can generate gene knockouts by disrupting the gene sequence, however, efficiency for inserting exogenous DNA (knock-in) or replacement of genomic sequences is very poor using current methods. In certain embodiments, CiSSDs may be used with CRISPR by generating a knock-in modification. Double-stranded breaks can be introduced by any suitable mechanism, including, for example, by gene-editing systems using CRISPR, zinc finger nuclease, TALEN nuclease (Transcription Activator-Like Effector Nuclease), or meganuclease as described previously. Briefly, the CRISPR genome editing system generates a targeted DSB using the CRISPR programmable DNA endonuclease that can be targeted to a specific DNA sequence (target sequence) by a small “guide” RNA (crRNA). Guide RNAs for use in CRISPR-based modification (z.e., crRNAs and tracrRNAs) may be generated by any suitable method. In certain embodiments, crRNAs and tracrRNAs may be chemically synthesized. In other embodiments, a single guide RNA (sgRNA) may be constructed and synthesized by in vitro transcription.

In certain embodiments, an LNP of the present disclosure comprises a CiSSD disclosed herein and further comprises a precision gene editing system component such as a CRISPR, zinc finger nuclease, TALEN nuclease (Transcription Activator-Like Effector Nuclease), or meganuclease, or any other nucleobase editing system known in the art.

In certain embodiments, the single stranded DNA (SSD) payload is one described in PCT Publication WO2020232286A1, which is incorporated by reference herein in its entirety.

In certain embodiments, the SSD comprises an engineered initiator sequence and an engineered terminator sequence from a filamentous bacteriophage, and a DNA sequence of interest, wherein the DNA sequence of interest is located 3′ to the engineered initiator sequence and 5′ to the engineered terminator sequence. In certain embodiments, the SSD comprises a selectable marker.

In certain embodiments, the single stranded DNA (SSD) payload is made by a method described in PCT Publication WO2020232286A1. In certain embodiments, the SSD is made by a method comprising: (a) culturing a host cell of claim 11 under conditions suitable for producing a ssDNA from the DNA sequence of interest in the engineered nucleic acid and the plurality of bacteriophage proteins from the nucleic acid helper plasmid; (b) allowing the ssDNA and the plurality of bacteriophage proteins to assemble into an engineered phage; and (c) collecting the engineered phage. In certain embodiments, the method further comprises extracting the SSD from the engineered phage.

In certain embodiments, at least 90% of the SSD is the same length as the DNA sequence of interest. In certain embodiments, at least 95% of the ssDNA is the same length as the DNA sequence of interest. In certain embodiments, the SSD is between 100 and 20,000 nucleotides in length. In certain embodiments, the ssDNA is circular.

In certain embodiments, the single stranded DNA (SSD) payload is one described in PCT Publication WO2022011082A1, which is incorporated by reference herein in its entirety. In certain embodiments, the SSD comprises a first sequence from a filamentous bacteriophage, the first sequence having both initiator and terminator functions; a second sequence that is identical to the first sequence; and a single-strand DNA sequence of interest that is located between the first sequence and the second sequence. In certain embodiments, the SSD further comprises a selectable marker. In certain embodiments, the SSD is circular. In certain embodiments, the SSD is linear.

In certain embodiments, the single stranded DNA (SSD) payload is made by a method described in PCT Publication WO2022011082A1. In certain embodiments, the method comprises culturing a host cell under conditions suitable for producing the single-stranded DNA from the single-strand DNA sequence of interest in the isolated nucleic acid and producing the bacteriophage proteins from the nucleic acid helper plasmid; allowing the single-stranded DNA and bacteriophage proteins to assemble into an engineered phage; and collecting the engineered phage. In certain embodiments, the host cell comprises an isolated nucleic acid that includes: a first sequence from a filamentous bacteriophage, the first sequence having both initiator and terminator functions; a second sequence that is identical to the first sequence; and a single-strand DNA sequence of interest that is located between the first sequence and the second sequence, and a nucleic acid helper plasmid for expressing bacteriophage proteins capable of assembling a single-strand DNA into a bacteriophage. In certain embodiments, the method further comprises extracting the SSD from the engineered phage.

In certain embodiments, at least 90% of the SSD is the same length as the DNA sequence of interest. In certain embodiments, at least 95% of the ssDNA is the same length as the DNA sequence of interest. In certain embodiments, the SSD is between 100 and 20,000 nucleotides in length. In certain embodiments, the SSD is circular.

In certain embodiments, the single stranded DNA (SSD) payload is one described in PCT Publication WO2021055616A1, which is incorporated by reference herein in its entirety.

3. Linear mRNA Payloads

In various embodiments, the LNP-based pharmaceutical compositions described herein, e.g., LNP-based gene editing systems, may include one or more linear mRNA molecules or linear mRNA payloads. In various embodiments, the mRNA payloads may encode one or more components of the herein described gene editing systems. For example, an mRNA payload may encode an amino acid sequence-programmable DNA binding domain (e.g., retron RTs or programmable nucleases) or a nucleic acid sequence-programmable DNA binding domain (e.g., CRISPR Cas9, CRISPR Cas12a, CRISPR Cas12f, CRISPR Cas13a, CRISPR Cas13b, or TnpB).

mRNA payloads may also encode, depending upon the nature of the gene editing system, one or more effector domains that provide various functionalities that facilitate changes in nucleotide sequence and/or gene expression, such as, but not limited to, single-strand DNA binding proteins, nucleases, endonucleases, exonucleases, deaminases (e.g., cytidine deaminases or adenosine deaminases), polymerases (e.g., reverse transcriptases), integrases, recombinases, etc., and fusion proteins comprising one or more functional domains linked together.

Ribonucleic acid (RNA) is a molecule that is made up of nucleotides, which are ribose sugars attached to nitrogenous bases and phosphate groups. The nitrogenous bases include adenine (A), guanine (G), uracil (U), and cytosine (C). Generally, RNA mostly exists in the single-stranded form but can also exists double-stranded in certain circumstances. The length, form and structure of RNA is diverse depending on the purpose of the RNA. For example, the length of an RNA can vary from a short sequence (e.g., siRNA) to a long sequences (e.g., lncRNA), can be linear (e.g., mRNA) or circular (e.g., oRNA), and can either be a coding (e.g., mRNA) or a non-coding (e.g., lncRNA) sequence.

In various embodiments, the LNP-based gene editing systems, RNA therapeutics and pharmaceutical compositions thereof described herein can be used to deliver a mRNA payload that is a linear mRNA molecule. In embodiments, the mRNA payload may comprise one or more nucleotide sequences that encode a product of interest, such as, but not limited to a component of a gene editing system (e.g., an endonuclease, a prime editor, etc.) and/or a therapeutic protein.

In some embodiments, the RNA payload may be a linear mRNA. As used herein, the term “messenger RNA” (mRNA) refers to any polynucleotide which encodes a protein of interest and which is capable of being translated to produce the encoded protein of interest in vitro, in vivo, in situ or ex vivo.

Generally, a mRNA molecule comprises at least a coding region, a 5′ untranslated region (UTR), a 3′ UTR, a 5′ cap and a poly-A tail. In some aspects, one or more structural and/or chemical modifications or alterations may be included in the RNA which can reduce the innate immune response of a cell in which the mRNA is introduced. As used herein, a “structural” feature or modification is one in which two or more linked nucleotides are inserted, deleted, duplicated, inverted or randomized in a nucleic acid without significant chemical modification to the nucleotides themselves. Because chemical bonds will necessarily be broken and reformed to affect a structural modification, structural modifications are of a chemical nature and hence are chemical modifications. However, structural modifications will result in a different sequence of nucleotides. For example, the polynucleotide “ATCG” may be chemically modified to “AT-5meC-G”.

Generally, a coding region of interest in an mRNA used herein may encode a dipeptide, a tripeptide, a tetrapeptide, a pentapeptide, a hexapeptide, a heptapeptide, an octapeptide, a nonapeptide, or a decapeptide. In another embodiment, the mRNA may encode a peptide of 2-30 amino acids, e.g. 5-30, 10-30, 2-25, 5-25, 10-25, or 10-20 amino acids. The mRNA may encode a peptide of at least 10, 11, 12, 13, 14, 15, 17, 20, 25, 26, 27, 28, 29, 30, 31, 32, 33, 34, 35, 36, 37, 38, 39, or 40 amino acids, or a peptide that is no longer than 10, 11, 12, 13, 14, 15, 17, 20, 25, 26, 27, 28, 29, 30, 31, 32, 33, 34, 35, 36, 37, 38, 39, or 40 amino acids.

Generally, the length of the region of the mRNA encoding a product of interest is greater than about 30 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1000, 1,100, 1,200, 1,300, 1,400, 1,500, 1,600, 1,700, 1,800, 1,900, 2,000, 2,500, and 3,000, 4,000, 5,000, 6,000, 7,000, 8,000, 9,000, 10,000, 20,000, 30,000, 40,000, 50,000, 60,000, 70,000, 80,000, 90,000 or up to and including 100,000 nucleotides).

In some embodiments, the mRNA has a total length that spans from about 30 to about 100,000 nucleotides (e.g., from 30 to 50, from 30 to 100, from 30 to 250, from 30 to 500, from 30 to 1,000, from 30 to 1,500, from 30 to 3,000, from 30 to 5,000, from 30 to 7,000, from 30 to 10,000, from 30 to 25,000, from 30 to 50,000, from 30 to 70,000, from 100 to 250, from 100 to 500, from 100 to 1,000, from 100 to 1,500, from 100 to 3,000, from 100 to 5,000, from 100 to 7,000, from 100 to 10,000, from 100 to 25,000, from 100 to 50,000, from 100 to 70,000, from 100 to 100,000, from 500 to 1,000, from 500 to 1,500, from 500 to 2,000, from 500 to 3,000, from 500 to 5,000, from 500 to 7,000, from 500 to 10,000, from 500 to 25,000, from 500 to 50,000, from 500 to 70,000, from 500 to 100,000, from 1,000 to 1,500, from 1,000 to 2,000, from 1,000 to 3,000, from 1,000 to 5,000, from 1,000 to 7,000, from 1,000 to 10,000, from 1,000 to 25,000, from 1,000 to 50,000, from 1,000 to 70,000, from 1,000 to 100,000, from 1,500 to 3,000, from 1,500 to 5,000, from 1,500 to 7,000, from 1,500 to 10,000, from 1,500 to 25,000, from 1,500 to 50,000, from 1,500 to 70,000, from 1,500 to 100,000, from 2,000 to 3,000, from 2,000 to 5,000, from 2,000 to 7,000, from 2,000 to 10,000, from 2,000 to 25,000, from 2,000 to 50,000, from 2,000 to 70,000, and from 2,000 to 100,000 nucleotides).

In some embodiments, the region or regions flanking the region encoding the product of interest may range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, and 900 nucleotides or at least 30, 40, 45, 50, 55, 60, 70, 80, 90, 100, 120, 140, 160, 180, 200, 250, 300, 350, 400, 450, 500, 600, 700, 800, 900, and 1,000 nucleotides).

In some embodiments, the mRNA comprises a tailing sequence which can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length may be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

In some embodiments, the mRNA comprises a capping sequence which comprises a single cap or a series of nucleotides forming the cap. The capping sequence may be from 1 to 10, e.g. 2-9, 3-8, 4-7, 1-5, 5-10, or at least 2, or 10 or fewer nucleotides in length. In some embodiments, the caping sequence is absent.

In some embodiments, the mRNA comprises a region comprising a start codon. The region comprising the start codon may range from 3 to 40, e.g., 5-30, 10-20, 15, or at least 4, or 30 or fewer nucleotides in length.

In some embodiments, the mRNA comprises a region comprising a stop codon. The region comprising the stop codon may range from 3 to 40, e.g., 5-30, 10-20, 15, or at least 4, or 30 or fewer nucleotides in length.

In some embodiments, the mRNA comprises a region comprising a restriction sequence. The region comprising the restriction sequence may range from 3 to 40, e.g., 5-30, 10-20, 15, or at least 4, or 30 or fewer nucleotides in length.

### Untranslated Regions (UTRs)

In various embodiments, the mRNA payloads of the LNP-based nucleobase editing systems, RNA therapeutics and pharmaceutical compositions thereof described herein, may comprise at least one untranslated region (UTR) which flanks the region encoding the product of interest and/or is incorporated within the mRNA molecule. UTRs are transcribed by not translated. The mRNA payloads can include 5′ UTR sequences and 3′ UTR sequences, as well as internal UTRs.

The RNA payloads of the present disclosure may comprise one or more regions or parts which act or function as an untranslated region. Where nucleic acids are designed to encode at least one polypeptide of interest, the nucleic acid may comprise one or more of these untranslated regions (UTRs). Wild-type untranslated regions of a nucleic acid are transcribed but not translated. In mRNA, the 5′ UTR starts at the transcription start site and continues to the start codon but does not include the start codon; whereas, the 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the RNA payload molecules (e.g., linear and circular mRNA molecules) of the present disclosure to, among other things, enhance the stability of the molecule. The specific features can also be incorporated to ensure controlled down-regulation of the transcript in case they are misdirected to undesired organs sites. A variety of 5′UTR and 3′UTR sequences are known and available in the art.

In various embodiments, the mRNA payloads of the LNP-based nucleobase editing systems, RNA therapeutics and pharmaceutical compositions thereof described herein, may comprise at least one UTR that may be selected from any UTR sequence listed in Tables 19 or 20 of U.S. Pat. No. 10,709,779, which is incorporated herein by reference.

### 5′ UTR Regions

In various embodiments, the mRNA payloads of the LNP-based gene editing systems, RNA therapeutics and pharmaceutical compositions thereof described herein, may comprise at least one 5′ UTR.

A 5′ UTR is region of an mRNA that is directly upstream (5′) from the start codon (the first codon of an mRNA transcript translated by a ribosome). A 5′ UTR does not encode a protein (is non-coding). Natural 5′UTRs have features that play roles in translation initiation. They harbor signatures like Kozak sequences which are commonly known to be involved in the process by which the ribosome initiates translation of many genes. Kozak sequences have the consensus CCR(A/G)CCAUGG (SEQ ID NO: 19421), where R is a purine (adenine or guanine) three bases upstream of the start codon (AUG), which is followed by another ‘G’. 5′UTR also have been known to form secondary structures which are involved in elongation factor binding. 5′ UTR sequences are also known to be important for ribosome recruitment to the mRNA and have been reported to play a role in translation (Hinnebusch A, et al., (2016) Science, 352:6292: 1413-6). In addition, 5′ UTR sequences may confer increased half-life, increased expression and/or increased activity of a polypeptide encoded by the RNA payload described herein.

In various embodiments, the RNA payload constructs contemplated herein may include 5′UTRs that are found in nature and those that are not. For example, the 5′UTRs can be synthetic and/or can be altered in sequence with respect to a naturally occurring 5′UTR. Such altered 5′UTRs can include one or more modifications relative to a naturally occurring 5′UTR, such as, for example, an insertion, deletion, or an altered sequence, or the substitution of one or more nucleotide analogs in place of a naturally occurring nucleotide.

The 5′ UTR starts at the transcription start site and continues to the start codon but does not include the start codon; whereas, the 3′UTR starts immediately following the stop codon and continues until the transcriptional termination signal. While not wishing to be bound by theory, the UTRs may have a regulatory role in terms of translation and stability of the nucleic acid.

Natural 5′ UTRs usually include features which have a role in translation initiation as they tend to include Kozak sequences which are commonly known to be involved in the process by which the ribosome initiates translation of many genes. Kozak sequences have the consensus CCR(AuG)CCAUGG (SEQ TD NO: 19421), where R is a purine (adenine or guanine) three bases upstream of the start codon (AUG), which is followed by another ‘G’. 5′UTR also have been known to form secondary structures which are involved in elongation factor binding.

In an embodiment, the 5′ UTR comprises a sequence provided in Table X or a sequence having at least 80%, 85%, 90%, 95%, 96%, 97%, 98%, 99% or 100% sequence identity to a 5′ UTR sequence provided in Table Y or a variant or a fragment thereof (e.g., a fragment that lacks the first one, two, three, four, five, or six nucleotides of the 5′ UTR sequence provided in Table Y). In an embodiment, the 5′ UTR comprises a sequence with at least 80%, 85%, 90%, 95% 96%, 97%, 98%, 99% or 100% identity to any one of the sequences of Table Y.

TABLE Y
Exemplary nucleotide sequences of 5′ UTRs

| 5′ UTR Nucleotide Sequence | Sequence Identifier |
|---|---|
| ggaaaucgca aaauuugcuc uucgcguuag auuucuuuua guuuucucgc | SEQ ID NO: 19422 |
| aacuagcaag cuuuuuguuc ucgccgccgc c | |
| ggaaaucgca aaauuugcuc uucgcguuag auuucuuuua guuuucucgc | SEQ ID NO: 19423 |
| aacuagcaag cuuuuuguuc ucgccgccgc c | |
| ggaaaucgca aaauuugcuc uucgcguuag auuucuuuua guuuucuuuc | SEQ ID NO: 19424 |
| aacuagcaag cuuuuuguuc ucgccgccgc c | |
| ggaaaucgca aaauuugcuc ucuuuuucgc guuagauuuc uuuuaguuuu | SEQ ID NO: 19425 |
| cuykcaacua gcaagcuuuu uguucucgcc rcc | |
| ggaaaucccc acaaccgccu cauauccagg cucaagaaua gagcucagug | SEQ ID NO: 19426 |
| uuuuguuguu uaaucauucc gacguguuuu gcgauauucg cgcaaagcag | |
| ccagucgcgc gcuugcuuuu aaguagaguu guuuuuccac ccguuugcca | |
| ggcaucuuua auuuaacaua uuuuuuuuu ucagguaac cuacgccgcc | |
| acc | |
| ggaaauaaga gagaaaagaa gaguaagaag aaauauaaga ucucccugag | SEQ ID NO: 19427 |
| cuucagggag ccccggcgcc gccacc | |
| ggaaaccccc cacccccgua agagagaaaa gaagaguaag aagaaauaua | SEQ ID NO: 19428 |
| agaucucccu gagcuucagg gagccccggc gccgccacc | |
| ggagaacuuc cgcuuccguu gggcaagg cuuuuuuuuuuu uucugcuacc | SEQ ID NO: 19429 |
| gugacuaag | |
| ggaaauaaga gagaaaagaa gaguaagaag aaauauaaga gccacc | SEQ ID NO: 19430 |
| ggaaauaaga gagaaaagaa gaguaagaag aaauauaaga ccccggcgcc | SEQ ID NO: 19431 |
| gccacc | |
| ggaaacuuua uuuaguguua cuuuauuuuc uguuuauuug uguuucuuca | SEQ ID NO: 19432 |
| guggguuugu ucuaauuucc uuggccgcc | |
| ggaaaaucug uauuagguug gcguguucuu uggucgguug ggaguauugu | SEQ ID NO: 19433 |
| uguugauucg uuuguggucg guugccgcc | |
| ggaaaauuau uaacaucuug guauucucga uaaccauucg uuggauuuua | SEQ ID NO: 19434 |
| uuguauucgu aguuuggguu ccugccgcc | |
| ggaaauuauu auuauuucua guacaauuu aucauuguau uuuuuacu | SEQ ID NO: 19435 |
| auucaucauu auuuacuugg ugaucaaca | |
| ggaaauaggu uguuaaccaa guucaagccu aauaagcuug gauucuggug | SEQ ID NO: 19436 |
| acuugcuuca ccguuggogg gcaccgauc | |
| ggaaaucgua gagagucgua cuuaguacau aucgacuauc gguggacacc | SEQ ID NO: 19437 |
| aucaagauua uaaaccaggc caga | |
| ggaaacccgc ccaagcgacc ccaacauauc agcaguugcc caaucccaac | SEQ ID NO: 19438 |
| ucccaacaca auccccaagc aacgccgcc | |
| ggaaagcgau ugaaggcguc uuuucaacua cucgauuaag guuggguauc | SEQ ID NO: 19439 |
| gucgugggac uuggaaauuu guuguuucc | |
| ggaaacuaau cgaaauaaaa gagccccgua cucuuuuauu ucuauuaggu | SEQ ID NO: 19440 |
| uaggagccuu agcauuugua ucuuaggua | |
| ggaaauguga uuuccagcaa cuucuuuuga auauauugaa uuccuaauuc | SEQ ID NO: 19441 |
| aaagcgaaca aaucuacaag ccauauacc | |
| ggaaaucgua gagagucgua cuuacguggu cgccauugca uagcgcgcga | SEQ ID NO: 19442 |
| aagcaacagg aacaagaacg cgcc | |
| ggaaaucgua gagagucgua cuuagaauaa acagagucgg gucgacuugu | SEQ ID NO: 19443 |
| cucugauacu acgacgucac aauc | |
| ggaaaauuug ccuucggagu ugcguauccu gaacugccca gccuccugau | SEQ ID NO: 19444 |
| auacaacugu uccgcuuauu cgggccgcc | |
| ggaaaucuga gcaggaaucc uuugugcauu gaagacuuua gauuccucuc | SEQ ID NO: 19445 |
| ugogguagac gugcacuuau aaguauuug | |
| ggaaagcgau ugaaggcguc uuuucaacua cucgauuaag guuggguauc | SEQ ID NO: 19446 |
| gucgugggac uuggaaauuu guugccacc | |
| ggaaaauuuu agccuggaac guuagauaac uguccuguug ucuuuauaua | SEQ ID NO: 19447 |
| cuuggucccc aaguaguuug ucuuccaaa | |
| ggaaauuuuu uuuugauauu auaagaguuu uuuuuugaua uuaagaaaau | SEQ ID NO: 19448 |
| uuuuuuuuga uauuagaaga guaagaagaa auauaagacc ccggcgccgc | |
| cacc | |
| ggaaauaaga gagaaaagaa gaguaagaag aaauauaaga gccaaaaaaa | SEQ ID NO: 19449 |
| aaaaacc | |
| ggaaaucucc cugagcuuca gggaguaaga gagaaaagaa gaguaagaag | SEQ ID NO: 19450 |
| aaauauaaga ccccggcgcc gccacc | |

In some embodiments of the disclosure, a 5′ UTR is a heterologous UTR, i.e., is a UTR found in nature associated with a different mRNA. In another embodiment, a 5′ UTR is a synthetic UTR, i.e., does not occur in nature. Synthetic UTRs include UTRs that have been mutated to improve their properties, e.g., which increase gene expression as well as those which are completely synthetic. Exemplary 5′ UTRs include Xenopus or human derived alpha-globin or beta-globin (e.g., U.S. Pat. Nos. 8,278,063 and 9,012,219), human cytochrome b-245 polypeptide, and hydroxysteroid (17b) dehydrogenase, and Tobacco etch virus. CMV immediate-early 1 (IE1) gene (see US20140206753 and WO2013/185069), the sequence GGGAUCCUACC (SEQ ID NO: 19451) (WO2014144196) may also be used. In another embodiment, 5′ UTR of a TOP gene is a 5′ UTR of a TOP gene lacking the 5′ TOP motif (the oligopyrimidine tract) (e.g., WO/2015101414, WO2015101415, WO/2015/062738, WO2015024667, WO2015024667; 5′ UTR element derived from ribosomal protein Large 32 (L32) gene (WO/2015101414, WO2015101415, WO/2015/062738)), 5′ UTR element derived from the 5′UTR of an hydroxysteroid (17-β) dehydrogenase 4 gene (HSD17B4) (WO2015024667), or a 5′ UTR element derived from the 5′ UTR of ATP5A1 (WO2015024667) can be used. In one embodiment, an internal ribosome entry site (IRES) is used as a substitute for a 5′ UTR.

In some embodiments, a 5′ UTR of the present disclosure comprises a sequence selected from SEQ ID NO:19452 (GGGAAAUAAG AGAGAAAAGA AGAGUAAGAA GAAAUAUAAG AGCCACC), and SEQ ID NO: 19453 (GGGAAATAAG AGAGAAAAGA AGAGTAAGAA GAAATATAAG AGCCACC).

### 3′ UTR Regions

In various embodiments, the mRNA payloads of the LNP-based base editing systems, RNA therapeutics and pharmaceutical compositions thereof described herein, may comprise at least one 3′ UTR. 3′ UTRs may be heterologous or synthetic.

A 3′ UTR is region of an mRNA that is directly downstream (3′) from the stop codon (the codon of an mRNA transcript that signals a termination of translation). A 3′ UTR does not encode a protein (is non-coding). Natural or wild type 3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

3′ UTRs are known to have stretches of adenosines and uridines embedded in them. These AU rich signatures are particularly prevalent in genes with high rates of turnover. Based on their sequence features and functional properties, the AU rich elements (AREs) can be separated into three classes (Chen et al., 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-α. Class III ARES are less well defined. These U rich regions do not contain an AUUUA motif. c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of the mRNA payloads described herein. For example, one or more copies of an ARE can be introduced to make mRNA less stable and thereby curtail translation and decrease production of the resultant protein. Alternatively, AREs can be identified and removed or mutated to increase the intracellular stability and thus increase translation and production of the resultant protein.

In some embodiments, the introduction of features often expressed in genes of target organs the stability and protein production of the mRNA can be enhanced in a specific organ and/or tissue. As a non-limiting example, the feature can be a UTR. As another example, the feature can be introns or portions of introns sequences.

Those of ordinary skill in the art will understand that 5′ UTRs that are heterologous or synthetic may be used with any desired 3′ UTR sequence. For example, a heterologous 5′ UTR may be used with a synthetic 3′ UTR with a heterologous 3′ UTR.

Non-UTR sequences may also be used as regions or subregions within an RNA payload construct. For example, introns or portions of introns sequences may be incorporated into regions of nucleic acid of the disclosure. Incorporation of intronic sequences may increase protein production as well as nucleic acid levels.

Combinations of features may be included in flanking regions and may be contained within other features. For example, the polypeptide coding region of interest in an mRNA payload may be flanked by a 5′ UTR which may contain a strong Kozak translational initiation signal and/or a 3′ UTR which may include an oligo(dT) sequence for templated addition of a poly-A tail. 5′ UTR may comprise a first polynucleotide fragment and a second polynucleotide fragment from the same and/or different genes such as the 5′ UTRs described in US Patent Application Publication No. 20100293625 and PCT/US2014/069155, herein incorporated by reference in its entirety

It should be understood that any UTR from any gene may be incorporated into the regions of an RNA payload molecule (e.g., a linear mRNA). Furthermore, multiple wild-type UTRs of any known gene may be utilized. It is also within the scope of the present disclosure to provide artificial UTRs which are not variants of wild type regions. These UTRs or portions thereof may be placed in the same orientation as in the transcript from which they were selected or may be altered in orientation or location. Hence a 5′ or 3′ UTR may be inverted, shortened, lengthened, made with one or more other 5′ UTRs or 3′ UTRs. As used herein, the term “altered” as it relates to a UTR sequence, means that the UTR has been changed in some way in relation to a reference sequence. For example, a 3′ UTR or 5′ UTR may be altered relative to a wild-type or native UTR by the change in orientation or location as taught above or may be altered by the inclusion of additional nucleotides, deletion of nucleotides, swapping or transposition of nucleotides. Any of these changes producing an “altered” UTR (whether 3′ or 5′) comprise a variant UTR.

In some embodiments, a double, triple or quadruple UTR such as a 5′ UTR or 3′ UTR may be used. As used herein, a “double” UTR is one in which two copies of the same UTR are encoded either in series or substantially in series. For example, a double beta-globin 3′ UTR may be used as described in US Patent publication 20100129877, the contents of which are incorporated herein by reference in its entirety.

It is also within the scope of the present disclosure to have patterned UTRs. As used herein “patterned UTRs” are those UTRs which reflect a repeating or alternating pattern, such as ABABAB or AABBAABBAABB or ABCABCABC or variants thereof repeated once, twice, or more than 3 times. In these patterns, each letter, A, B, or C represent a different UTR at the nucleotide level.

In some embodiments, flanking regions are selected from a family of transcripts whose proteins share a common function, structure, feature or property. For example, polypeptides of interest may belong to a family of proteins which are expressed in a particular cell, tissue or at some time during development. The UTRs from any of these genes may be swapped for any other UTR of the same or different family of proteins to create a new polynucleotide. As used herein, a “family of proteins” is used in the broadest sense to refer to a group of two or more polypeptides of interest which share at least one function, structure, feature, localization, origin, or expression pattern.

The untranslated region may also include translation enhancer elements (TEE). As a non-limiting example, the TEE may include those described in US Application No. 20090226470, herein incorporated by reference in its entirety, and those known in the art.

### 5′ Capping

In various embodiments, the mRNA payloads of the LNP-based base editing systems, RNA therapeutics and pharmaceutical compositions thereof described herein, may comprise a 5′ cap structure.

The 5′ cap structure of an mRNA is involved in nuclear export, increasing mRNA stability and binds the mRNA Cap Binding Protein (CBP), which is responsible for mRNA stability in the cell and translation competency through the association of CBP with poly(A) binding protein to form the mature cyclic mRNA species. The cap further assists the removal of 5′ proximal introns removal during mRNA splicing.

Endogenous mRNA molecules may be 5′-end capped generating a 5′-ppp-5′-triphosphate linkage between a terminal guanosine cap residue and the 5′-terminal transcribed sense nucleotide of the mRNA molecule. This 5′-guanylate cap may then be methylated to generate an N7-methyl-guanylate residue. The ribose sugars of the terminal and/or anteterminal transcribed nucleotides of the 5′ end of the mRNA may optionally also be 2′-O-methylated. 5′-decapping through hydrolysis and cleavage of the guanylate cap structure may target a nucleic acid molecule, such as an mRNA molecule, for degradation.

Modifications to mRNA may generate a non-hydrolyzable cap structure preventing decapping and thus increasing mRNA half-life. Because cap structure hydrolysis requires cleavage of 5′-ppp-5′ phosphorodiester linkages, modified nucleotides may be used during the capping reaction. For example, a Vaccinia Capping Enzyme from New England Biolabs (Ipswich, MA) may be used with a-thio-guanosine nucleotides according to the manufacturer's instructions to create a phosphorothioate linkage in the 5′-ppp-5′ cap.

Additional modified guanosine nucleotides may be used such as a-methylphosphonate and seleno-phosphate nucleotides.

Additional modifications include, but are not limited to, 2-O-methylation of the ribose sugars of 5′-terminal and/or 5′-anteterminal nucleotides of the mRNA (as mentioned above) on the 2′-hydroxyl group of the sugar ring. Multiple distinct 5′-cap structures can be used to generate the 5′-cap of a nucleic acid molecule, such as an mRNA molecule.

Cap analogs, which herein are also referred to as synthetic cap analogs, chemical caps, chemical cap analogs, or structural or functional cap analogs, differ from natural (i.e. endogenous, wild-type or physiological) 5-caps in their chemical structure, while retaining cap function. Cap analogs may be chemically (i.e. non-enzymatically) or enzymatically synthesized and/or linked to a nucleic acid molecule.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which may equivalently be designated 3′ O-Me-m7G(5′)ppp(5′)G). The 3′-0 atom of the other, unmodified, guanine becomes linked to the 5-terminal nucleotide of the capped nucleic acid molecule (e.g. an mRNA). The N7- and 3′-O-methylated guanine provides the terminal moiety of the capped nucleic acid molecule (e.g. mRNA).

Another exemplary cap is mCAP, which is similar to ARCA but has a 2-O-methyl group on guanosine (i.e., N7,2′-O-dimethyl-guanosine-5′-triphosphate-5′-guanosine, m⁷Gm-ppp-G).

While cap analogs allow for the concomitant capping of a nucleic acid molecule in an in vitro transcription reaction, up to 20% of transcripts can remain uncapped. This, as well as the structural differences of a cap analog from an endogenous 5′-cap structures of nucleic acids produced by the endogenous, cellular transcription machinery, may lead to reduced translational competency and reduced cellular stability.

mRNA may also be capped post-transcriptionally, using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of an mRNA and a guanine cap nucleotide wherein the cap guanine contains an N7 methylation and the 5′-terminal nucleotide of the mRNA contains a 2-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5*)ppp(5*)N, pN2p (cap 0), 7mG(5*)ppp(5*)NlmpNp (cap 1), and 7mG(5*)-ppp(5′)NlmpN2mp (cap 2).

In some embodiments, the 5′ terminal caps may include endogenous caps or cap analogs.

In some embodiments, a 5′ terminal cap may comprise a guanine analog. Useful guanine analogs include, but are not limited to, inosine, Nl-methyl-guanosine, 2′fluoro-guanosine, 7-deaza-guanosine, 8-oxo-guanosine, 2-amino-guanosine, LNA-guanosine, and 2-azido-guanosine.

### IRES Sequences

In various embodiments, the mRNA payloads of the LNP-based gene editing systems, RNA therapeutics and pharmaceutical compositions thereof described herein, may comprise one or more IRES sequences.

In some embodiments, the mRNA may contain an internal ribosome entry site (IRES). First identified as a feature Picorna virus RNA, IRES plays an important role in initiating protein synthesis in absence of the 5′ cap structure. An IRES may act as the sole ribosome binding site, or may serve as one of multiple ribosome binding sites of an mRNA. An mRNA that contains more than one functional ribosome binding site may encode several peptides or polypeptides that are translated independently by the ribosomes. Non-limiting examples of IRES sequences that can be used include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (SIV) or cricket paralysis viruses (CrPV).

In some embodiments, the TRES is from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picorna-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human ATIR, Human BAG-1, Human BCL2, Human BiP, Human c-IAP1, Human c-myc, Human eIF4G, MouseNDST4L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NATOO, HRV14, HRV89, HRVC-02, HRV-A21, Salivirus A SHI, Salivirus FHB, Salivirus NG-JI, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV—PK15C, SF573 Dicistrovirus, Hubei Picorna-like Virus, CRPV, Salivirus A BNS, Salivirus A BN2, Salivirus A 02394, Salivirus A GUT, Salivirus A CH, Salivirus A SZ1, Salivirus FHB, CVB3, CVB1, Echovirus 7, CVBS, EVA71, CVA3, CVA12, EV24 or an aptamerto eIF4G.

### Poly-A Tails and 3′ Stabilizing Regions

In various embodiments, the mRNA payloads of the LNP-based gene editing systems, RNA therapeutics and pharmaceutical compositions thereof described herein, may comprise a poly-A tail.

During RNA processing, a long chain of adenine nucleotides (poly-A tail) may be added to a polynucleotide such as an mRNA molecules in order to increase stability. Immediately after transcription, the 3′ end of the transcript may be cleaved to free a 3′ hydroxyl. Then poly-A polymerase adds a chain of adenine nucleotides to the free 3′ hydroxyl end. The process, called polyadenylation, adds a poly-A tail of a certain length.

In some embodiments, the length of a poly-A tail is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides) and no more than about 50, 100, 200, 300, 400, 500, 600, 700, 800, 900, 1000, 2000, or 3000 nucleotides in length. In some embodiments, the mRNA includes a poly-A tail from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall mRNA. This design may be based on the length of the region coding for a target of interest, the length of a particular feature or region (such as a flanking region), or based on the length of the ultimate product expressed from the mRNA.

In this context the poly-A tail may be 10, 20, 30, 40, 50, 60, 70, 80, 90, or 100% greater in length than the mRNA or feature thereof. The poly-A tail may also be designed as a fraction of mRNA to which it belongs. In this context, the poly-A tail may be 10, 20, 30, 40, 50, 60, 70, 80, or 90% or more of the total length of the construct or the total length of the construct minus the poly-A tail. Further, engineered binding sites and conjugation of mRNA for poly-A binding protein may enhance expression.

Additionally, multiple distinct mRNA may be linked together to the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the mRNA are designed to include a polyA-G Quartet. The G-quartet is a cyclic hydrogen bonded array of four guanine nucleotides that can be formed by G-rich sequences in both DNA and RNA. In this embodiment, the G-quartet is incorporated at the end of the poly-A tail.

### Stop Codons

In various embodiments, the mRNA payloads of the LNP-based gene editing systems, RNA therapeutics and pharmaceutical compositions thereof described herein, may comprise one or more translation stop codons. Translational stop codons, UAA, UAG, and UGA, are an important component of the genetic code and signal the termination of translation of an mRNA. During protein synthesis, stop codons interact with protein release factors and this interaction can modulate ribosomal activity thus having an impact translation (Tate W P, et al., (2018) Biochem Soc Trans, 46(6):1615-162).

A stop element as used herein, refers to a nucleic acid sequence comprising a stop codon. The stop codon can be selected from TGA, TAA and TAG in the case of DNA, or from UGA, UAA and UAG in the case of RNA. In an embodiment, a stop element comprises two consecutive stop codons. In an embodiment, a stop element comprises three consecutive stop codons. In an embodiment, a stop element comprises four consecutive stop codons. In an embodiment, a stop element comprises five consecutive stop codons.

In some embodiments, the mRNA may include one stop codon. In some embodiments, the mRNA may include two stop codons. In some embodiments, the mRNA may include three stop codons. In some embodiments, the mRNA may include at least one stop codon. In some embodiments, the mRNA may include at least two stop codons. In some embodiments, the mRNA may include at least three stop codons. As non-limiting examples, the stop codon may be selected from TGA, TAA and TAG.

In other embodiments, the stop codon may be selected from one or more of the following stop elements of Table Z:

TABLE Z
Additional stop elements of linear mRNA

| Nucleotide sequence | |
|---|---|
| (5′ to 3′) | Sequence Identifier |
| UGAUAAUAG | SEQ ID NO: 19454 |
| UAAUAGUAA | SEQ ID NO: 19455 |
| UAAGUCUAA | SEQ ID NO: 19456 |
| UAAAGCUAA | SEQ ID NO: 19457 |
| UAAGUCUCC | SEQ ID NO: 19458 |
| UAAGGCUAA | SEQ ID NO: 19459 |
| UAAGCCCCUCCGGGG | SEQ ID NO: 19460 |
| UAAAGCUCCCCGGGG | SEQ ID NO: 19461 |
| UAAGCCCCU | SEQ ID NO: 19462 |
| UAAAGCUCC | SEQ ID NO: 19463 |
| UAAAGCUCC | SEQ ID NO: 19464 |
| UAGGGUUAA | SEQ ID NO: 19465 |
| UAAGCACCC | SEQ ID NO: 19466 |
| UGAUAGUAA | SEQ ID NO: 19467 |
| UAAAGCGCU | SEQ ID NO: 19468 |

In some embodiments, the mRNA includes the stop codon TGA and one additional stop codon. In a further embodiment the addition stop codon may be TAA.

### MicroRNA Binding Sites and Other Regulatory Elements

In various embodiments, the mRNA payloads of the LNP-based gene editing systems, RNA therapeutics and pharmaceutical compositions thereof described herein, may comprise one or more regulatory elements, including, but not limited to microRNA (miRNA) binding sites, structured mRNA sequences and/or motifs, artificial binding sites to bind to endogenous nucleic acid binding molecules, and combinations thereof.

### Chemically Unmodified Nucleotides

In some embodiments, the mRNA payloads of the LNP-based gene editing systems, RNA therapeutics and pharmaceutical compositions thereof described herein are not chemically modified and comprises the standard ribonucleotides consisting of adenosine, guanosine, cytosine and uridine. In some embodiments, nucleotides and nucleosides of the present disclosure comprise standard nucleoside residues such as those present in transcribed RNA (e.g. A, G, C, or U). In some embodiments, nucleotides and nucleosides of the present disclosure comprise standard deoxyribonucleosides such as those present in DNA (e.g. dA, dG, dC, or dT).

### Chemically Modified Nucleotides

In some embodiments, the mRNA payloads of the LNP-based gene editing systems, RNA therapeutics and pharmaceutical compositions thereof described herein comprise, in some embodiments, comprises at least one chemical modification.

The terms “chemical modification” and “chemically modified” refer to modification with respect to adenosine (A), guanosine (G), uridine (U), thymidine (T) or cytidine (C) ribonucleosides or deoxyribonucleosides in at least one of their position, pattern, percent or population. Generally, these terms do not refer to the ribonucleotide modifications in naturally occurring 5′-terminal mRNA cap moieties. With respect to a polypeptide, the term “modification” refers to a modification relative to the canonical set 20 amino acids. Polypeptides, as provided herein, are also considered “modified” if they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A “nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker may be incorporated into polynucleotides of the present disclosure.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m¹ψy), N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of 1-methyl-pseudouridine (m^{1ψ),} 5-methoxy-uridine (mo⁵U), 5-methyl-cytidine (m⁵C), pseudouridine (ψ), α-thio-guanosine and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (W) and 5-methyl-cytidine (m⁵C). In some embodiments, polynucleotides (e.g., RNA polynucleotides, such as mRNA polynucleotides) comprise 1-methyl-pseudouridine (m¹ψ). In some embodiments, polynucleotides (e.g., RNA polynucleotides, such as mRNA polynucleotides) comprise 1-methyl-pseudouridine (m¹ψ) and 5-methyl-cytidine (m⁵C). In some embodiments, polynucleotides (e.g., RNA polynucleotides, such as mRNA polynucleotides) comprise 2-thiouridine (s²U). In some embodiments, polynucleotides (e.g., RNA polynucleotides, such as mRNA polynucleotides) comprise 2-thiouridine and 5-methyl-cytidine (m⁵C). In some embodiments, polynucleotides (e.g., RNA polynucleotides, such as mRNA polynucleotides) comprise methoxy-uridine (mo⁵U). In some embodiments, polynucleotides (e.g., RNA polynucleotides, such as mRNA polynucleotides) comprise 5-methoxy-uridine (mo⁵U) and 5-methyl-cytidine (m⁵C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m⁵C). In some embodiments, polynucleotides (e.g., RNA polynucleotides, such as mRNA polynucleotides) comprise N6-methyl-adenosine (m⁶A). In some embodiments, polynucleotides (e.g., RNA polynucleotides, such as mRNA polynucleotides) comprise N6-methyl-adenosine (m⁶A) and 5-methyl-cytidine (mC).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m⁵C), meaning that all cytosine residues in the mRNA sequence are replaced with 5-methyl-cytidine (m⁵C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and In some embodiments, a modified nucleobase is a modified cytosine. nucleosides having a modified uridine include 5-cyano uridine, and 4′-thio uridine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the polyA tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

4. Circular mRNA Payloads

In various embodiments, the LNP-based pharmaceutical compositions described herein, e.g., LNP-based gene editing systems, may include one or more circular mRNA molecules or “oRNAs.” In various embodiments, the circular mRNA payloads may encode one or more components of the herein described gene editing systems or other therapeutic protein of interest. For example, a circular mRNA payload may encode an amino acid sequence-programmable DNA binding domain (e.g., retron RTs, CRISPR nucleases, TALENS and zinc finger-binding domains) or a nucleic acid sequence-programmable DNA binding domain (e.g., CRISPR Cas9, CRISPR Cas12a, CRISPR Cas12f, CRISPR Cas13a, CRISPR Cas13b, or TnpB).

The circular mRNA payloads may also encode, depending upon the nature of the gene editing system, one or more effector domains that provide various functionalities that facilitate changes in nucleotide sequence and/or gene expression, such as, but not limited to, single-strand DNA binding proteins, nucleases, endonucleases, exonucleases, deaminases (e.g., cytidine deaminases or adenosine deaminases), polymerases (e.g., reverse transcriptases), integrases, recombinases, etc., and fusion proteins comprising one or more functional domains linked together.

Circular RNA described herein are polyribonucleotides that form a continuous structure through covalent or non-covalent bonds. Due to the circular structure, oRNAs have improved stability, increased half-life, reduced immunogenicity, and/or improved functionality (e.g., of a function described herein) compared to a corresponding linear RNA.

In some embodiments, an oRNA binds a target. In some embodiments, an oRNA binds a substrate. In some embodiments, an oRNA binds a target and binds a substrate of the target. In some embodiments, an oRNA binds a target and mediates modulation of a substrate of the target. In some embodiments, an oRNA brings together a target and its substrate to mediate modification of the substrate, e.g., post-translational modification. In some embodiments, an oRNA brings together a target and its substrate to mediate a cellular process (e.g., alters protein degradation or signal transduction) involving the substrate. In some embodiments, a target is a target protein and a substrate is a substrate protein.

In some embodiments, an oRNA comprises a conjugation moiety for binding to chemical compound. The conjugation moiety can be a modified polyribonucleotide. The chemical compound can be conjugated to the oRNA by the conjugation moiety. In some embodiments, the chemical compound binds to a target and mediates modulation of a substrate of the target. In some embodiments, an oRNA binds a substrate of a target and a chemical compound conjugated to the oRNA by the conjugation moiety binds the target to bring together the target and its substrate to mediate modification of the substrate, e.g., post-translational modification. In some embodiments, an oRNA binds a substrate of a target and a chemical compound conjugated to the oRNA by the conjugation moiety binds the target to bring together the target and its substrate to mediate modification of the substrate to mediate a cellular process (e.g., alters protein degradation or signal transduction) involving the substrate. In some embodiments, a target is a target protein and a substrate is a substrate protein.

In some embodiments, the oRNA may be non-immunogenic in a mammal (e.g., a human, non-human primate, rabbit, rat, and mouse).

In some embodiments, the oRNA may be capable of replicating or replicates in a cell from an aquaculture animal (e.g., fish, crabs, shrimp, oysters etc.), a mammalian cell, a cell from a pet or zoo animal (e.g., cats, dogs, lizards, birds, lions, tigers and bears etc.), a cell from a farm or working animal (e.g., horses, cows, pigs, chickens etc.), a human cell, cultured cells, primary cells or cell lines, stem cells, progenitor cells, differentiated cells, germ cells, cancer cells (e.g., tumorigenic, metastatic), non-tumorigenic cells (e.g., normal cells), fetal cells, embryonic cells, adult cells, mitotic cells, non-mitotic cells, or any combination thereof.

In one aspect, provided herein is a pharmaceutical composition comprising: a circular RNA comprising, in the following order, a 3′ group I intron fragment, an Internal Ribosome Entry Site (IRES), an expression sequence encoding a polypeptide (e.g., a nucleobase editing system or component thereof), and a 5′ group I intron fragment, and a transfer vehicle comprising at least one of (i) an ionizable lipid, (ii) a structural lipid, and (iii) a PEG-modified lipid, wherein the transfer vehicle is capable of delivering the circular RNA polynucleotide to a cell (e.g., a human cell, such as an immune cell present in a human subject), such that the polypeptide is translated in the cell. In some embodiments, the pharmaceutical composition is formulated for intravenous administration to the human subject in need thereof. In some embodiments, the 3′ group I intron fragment and 5′ group I intron fragment are Anabaena group I intron fragments.

In certain embodiments, the 3′ intron fragment and 5′ intron fragment are defined by the L9a-5 permutation site in the intact intron. In certain embodiments, the 3′ intron fragment and 5′ intron fragment are defined by the L8-2 permutation site in the intact intron.

In some embodiments, the TRES is from Taura syndrome virus, Triatoma virus, Theiler's encephalomyelitis virus, Simian Virus 40, Solenopsis invicta virus 1, Rhopalosiphum padi virus, Reticuloendotheliosis virus, Human poliovirus 1, Plautia stall intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, Foot and mouth disease virus, Human enterovirus 71, Equine rhinitis virus, Ectropis obliqua picoma-like virus, Encephalomyocarditis virus, Drosophila C Virus, Human coxsackievirus B3, Crucifer tobamovirus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1/RUNX1, Drosophila antennapedia, Human AQP4, Human ATIR, Human BAG-1, Human BCL2, Human BiP, Human c-IAP1, Human c-myc, Human eIF4G, Mouse NDST4L, Human LEF1, Mouse HIF1 alpha, Human n.myc, Mouse Gtx, Human p27kipl, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Drosophila hairless, S. cerevisiae TFIID, S. cerevisiae YAP1, tobacco etch virus, turnip crinkle virus, EMCV-A, EMCV-B, EMCV-Bf, EMCV-Cf, EMCV pEC9, Picobirnavirus, HCV QC64, Human Cosavirus E/D, Human Cosavirus F, Human Cosavirus JMY, Rhinovirus NAT001, HRV14, HRV89, HRVC-02, HRV-A21, Salivirus A SHI, Salivirus FHB, Salivirus NG-JJ, Human Parechovirus 1, Crohivirus B, Yc-3, Rosavirus M-7, Shanbavirus A, Pasivirus A, Pasivirus A 2, Echovirus E14, Human Parechovirus 5, Aichi Virus, Hepatitis A Virus HA 16, Phopivirus, CVA10, Enterovirus C, Enterovirus D, Enterovirus J, Human Pegivirus 2, GBV-C GT110, GBV-C K1737, GBV-C Iowa, Pegivirus A 1220, Pasivirus A 3, Sapelovirus, Rosavirus B, Bakunsa Virus, Tremovirus A, Swine Pasivirus 1, PLV-CHN, Pasivirus A, Sicinivirus, Hepacivirus K, Hepacivirus A, BVDV1, Border Disease Virus, BVDV2, CSFV—PK15C, SF573 Dicistravirus, Hubei Picoma-like Virus, CRPV, Salivirus A BN5, Salivirus A BN2, Salivirus A 02394, Salivirus A GUT, Salivirus A CH, Salivirus A SZ1, Salivirus FHB, CVB3, CVB1, Echovirus 7, CVB5, EVA71, CVA3, CVA12, EV24 or an aptamer to eIF4G.

In some embodiments, the IRES comprises a CVB3 IRES or a fragment or variant thereof. In some embodiments, the pharmaceutical composition comprises a first internal spacer between the 3′ group I intron fragment and the TRES, and a second internal spacer between the expression sequence and the 5′ group I intron fragment. In certain embodiments, the first and second internal spacers each have a length of about 10 to about 60 nucleotides.

In some embodiments, the circular mRNA comprises a nucleotide sequence encoding a polypeptide of interest, such as a nucleobase editing system or therapeutic protein (e.g., a CAR or TCR complex protein).

In some embodiments, the LNP-based nucleobase editing systems, RNA therapeutics and pharmaceutical compositions described herein further comprise a targeting moiety. In certain embodiments, the targeting moiety mediates receptor-mediated endocytosis or direct fusion of the delivery vehicle (LNPs) into selected cells of a selected cell population or tissue in the absence of cell isolation or purification. In certain embodiments, the targeting moiety is capable of binding to a protein selected from the group CD3, CD4, CD8, CDS, CD7, PD-1, 4-1BB, CD28, Clq, and CD2. In certain embodiments, the targeting moiety comprises an antibody specific for a macrophage, dendritic cell, NK cell, NKT, or T cell antigen. In certain embodiments, the targeting moiety comprises a scFv, nanobody, peptide, minibody, polynucleotide aptamer, heavy chain variable region, light chain variable region or fragment thereof.

In some embodiments, the LNP-based nucleobase editing systems, RNA therapeutics and pharmaceutical compositions described herein are administered in an amount effective to treat a disease in the human subject (e.g., wherein the disease can be cancer, muscle disorder, or CNS disorder, etc.). In some embodiments, the LNP-based nucleobase editing systems, RNA therapeutics and pharmaceutical compositions have an enhanced safety profile when compared to a pharmaceutical composition comprising T cells or vectors comprising exogenous DNA encoding the same polypeptide.

In some embodiments, the LNP-based nucleobase editing systems and pharmaceutical compositions thereof are administered in an amount effective to induce a desire precise edit in a genome. In some embodiments, the LNP-based nucleobase editing systems and pharmaceutical compositions have an enhanced safety profile when compared to state of the art gene editing delivery compositions.

In another aspect, the present disclosure provides a circular RNA comprising, in the following order, a 3′ group I intron fragment, an Internal Ribosome Entry Site (IRES), an expression sequence encoding a polypeptide (e.g., a nucleobase editing system or component thereof), and a 5′ group I intron fragment.

In some embodiments, the 3′ group I intron fragment and 5′ group I intron fragment are Anabaena group I intron fragments. In certain embodiments, the 3′ intron fragment and 5′ intron fragment are defined by the L9a-5 permutation site in the intact intron. In certain embodiments, the 3′ intron fragment and 5′ intron fragment are defined by the L8-2 permutation site in the intact intron. In certain embodiments, the IRES comprises a CVB3 IRES or a fragment or variant thereof.

In some embodiments, the circular RNA comprises a first internal spacer between the 3′ group I intron fragment and the IRES, and a second internal spacer between the expression sequence and the 5′ group I intron fragment.

In certain embodiments, the first and second internal spacers each have a length of about 10 to about 60 nucleotides.

In some embodiments, the circular RNA payload of the LNP-based nucleobase editing systems, RNA therapeutics and pharmaceutical compositions described herein consists of natural nucleotides. In some embodiments, the circular RNA further comprises a second expression sequence encoding a therapeutic protein. In some embodiments, the therapeutic protein comprises a checkpoint inhibitor. In certain embodiments, the therapeutic protein comprises a cytokine.

In some embodiments, the circular RNA payload of the LNP-based nucleobase editing systems, RNA therapeutics and pharmaceutical compositions described herein consists of natural nucleotides.

In some embodiments, the circular RNA payload LNP-based nucleobase editing systems, RNA therapeutics and pharmaceutical compositions described herein comprises a nucleotide sequence that is codon optimized, either partially or fully. In some embodiments, the circular RNA is optimized to lack at least one microRNA binding site present in an equivalent pre-optimized polynucleotide. In some embodiments, the circular RNA is optimized to lack at least one endonuclease susceptible site present in an equivalent pre-optimized polynucleotide. In some embodiments, the circular RNA is optimized to lack at least one RNA-editing susceptible site present in an equivalent pre-optimized polynucleotide.

In some embodiments, the circular RNA payload of the LNP-based nucleobase editing systems, RNA therapeutics and pharmaceutical compositions described herein has an in vivo functional half-life in humans greater than that of an equivalent linear RNA having the same expression sequence. In some embodiments, the circular RNA has a length of about 100 nucleotides to about 10 kilobases. In some embodiments, the circular RNA has a functional half-life of at least about 20 hours. In some embodiments, the circular RNA has a duration of therapeutic effect in a human cell of at least about 20 hours. In some embodiments, the circular RNA has a duration of therapeutic effect in a human cell greater than or equal to that of an equivalent linear RNA comprising the same expression sequence. In some embodiments, the circular RNA has a functional half-life in a human cell greater than or equal to that of an equivalent linear RNA comprising the same expression sequence.

In some embodiments, the circular RNA payload of the LNP-based nucleobase editing systems, RNA therapeutics and pharmaceutical compositions described herein has a half-life of at least that of a linear counterpart. In some embodiments, the oRNA has a half-life that is increased over that of a linear counterpart. In some embodiments, the half-life is increased by about 5%, 10%, 15%, 20%, 25%, 30%, 35%, 40%, 45%, 50%, or greater. In some embodiments, the oRNA has a half-life or persistence in a cell for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In some embodiments, the oRNA has a half-life or persistence in a cell for no more than about 10 mins to about 7 days, or no more than about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 24 hours (1 day), 36 hours (1.5 days), 48 hours (2 days), 60 hours (2.5 days), 72 hours (3 days), 4 days, 5 days, 6 days, or 7 days.

In some embodiments, the circular RNA payload of the LNP-based nucleobase editing systems, RNA therapeutics and pharmaceutical compositions described herein has a half-life or persistence in a cell while the cell is dividing. In some embodiments, the oRNA has a half-life or persistence in a cell post division.

In certain embodiments, the circular RNA payload of the LNP-based nucleobase editing systems, RNA therapeutics and pharmaceutical compositions described herein has a half-life or persistence in a dividing cell for greater than about 10 minutes to about 30 days, or at least about 10 minutes, 15 minutes, 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, the circular RNA payload of the LNP-based nucleobase editing systems, RNA therapeutics and pharmaceutical compositions described herein modulates a cellular function, e.g., transiently or long term. In certain embodiments, the cellular function is stably altered, such as a modulation that persists for at least about 1 hour to about 30 days, or at least about 2 hours, 6 hours, 12 hours, 18 hours, 24 hours (1 day), 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer. In certain embodiments, the cellular function is transiently altered, e.g., such as a modulation that persists for no more than about 30 mins to about 7 days, or no more than about 30 minutes, 45 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours (1 day), 36 hours (1.5 days), 48 hours (2 days), 60 hours (2.5 days), 72 hours (3 days), 4 days, 5 days, 6 days, or 7 days.

In some embodiments, the circular RNA payload of the LNP-based nucleobase editing systems, RNA therapeutics and pharmaceutical compositions described herein is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the oRNA may be of a sufficient size to accommodate a binding site for a ribosome.

In some embodiments, the maximum size of the circular RNA payload of the LNP-based nucleobase editing systems, RNA therapeutics and pharmaceutical compositions described herein may be limited by the ability of packaging and delivering the RNA to a target. In some embodiments, the size of the oRNA is a length sufficient to encode polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides may be useful.

In some embodiments, the circular RNA payload of the LNP-based nucleobase editing systems, RNA therapeutics and pharmaceutical compositions described herein comprises one or more elements described elsewhere herein. In some embodiments, the elements may be separated from one another by a spacer sequence or linker. In some embodiments, the elements may be separated from one another by 1 nucleotide, 2 nucleotides, about 5 nucleotides, about 10 nucleotides, about 15 nucleotides, about 20 nucleotides, about 30 nucleotides, about 40 nucleotides, about 50 nucleotides, about 60 nucleotides, about 80 nucleotides, about 100 nucleotides, about 150 nucleotides, about 200 nucleotides, about 250 nucleotides, about 300 nucleotides, about 400 nucleotides, about 500 nucleotides, about 600 nucleotides, about 700 nucleotides, about 800 nucleotides, about 900 nucleotides, about 1000 nucleotides, up to about 1 kb, at least about 1000 nucleotides.

In some embodiments, one or more elements are contiguous with one another, e.g., lacking a spacer element.

In some embodiments, one or more elements is conformationally flexible. In some embodiments, the conformational flexibility is due to the sequence being substantially free of a secondary structure.

In some embodiments, the circular RNA payload of the LNP-based nucleobase editing systems, RNA therapeutics and pharmaceutical compositions described herein comprises a secondary or tertiary structure that accommodates a binding site for a ribosome, translation, or rolling circle translation.

In some embodiments, the circular RNA payload of the LNP-based nucleobase editing systems, RNA therapeutics and pharmaceutical compositions described herein comprises particular sequence characteristics. For example, the oRNA may comprise a particular nucleotide composition. In some such embodiments, the oRNA may include one or more purine rich regions (adenine or guanosine). In some such embodiments, the oRNA may include one or more purine rich regions (adenine or guanosine). In some embodiments, the oRNA may include one or more AU rich regions or elements (AREs). In some embodiments, the oRNA may include one or more adenine rich regions.

In some embodiments, the circular RNA payload of the LNP-based nucleobase editing systems, RNA therapeutics and pharmaceutical compositions described herein comprises one or more modifications described elsewhere herein.

In some embodiments, the circular RNA payload of the LNP-based nucleobase editing systems, RNA therapeutics and pharmaceutical compositions described herein comprises one or more expression sequences and is configured for persistent expression in a cell of a subject in vivo. In some embodiments, the oRNA is configured such that expression of the one or more expression sequences in the cell at a later time point is equal to or higher than an earlier time point. In such embodiments, the expression of the one or more expression sequences can be either maintained at a relatively stable level or can increase over time. The expression of the expression sequences can be relatively stable for an extended period of time. For instance, in some cases, the expression of the one or more expression sequences in the cell over a time period of at least 7, 8, 9, 10, 12, 14, 16, 18, 20, 22, 23 or more days does not decrease by 50%, 45%, 40%, 35%, 30%, 25%, 20%, 15%, 10%, or 5%. In some cases, in some cases, the expression of the one or more expression sequences in the cell is maintained at a level that does not vary by more than 50%, 45%, 40%, 35%, 30%, 25%, 20%, 15%, 10%, or 5% for at least 7, 8, 9, 10, 12, 14, 16, 18, 20, 22, 23 or more days.

### Regulatory Elements

In some embodiments, the circular RNA payload of the LNP-based nucleobase editing systems, RNA therapeutics and pharmaceutical compositions described herein comprises one or more regulatory elements. As used herein, a “regulatory element” is a sequence that modifies expression of an expression sequence, e.g., a nucleotide sequence encoding a nucleobase editing system or a therapeutic protein, i.e., a coding region of interest (CROI). The regulatory element may include a sequence that is located adjacent to a coding region of interest encoded on the circular RNA payload. The regulatory element may be operatively linked to a nucleotide sequence of the circular RNA that encodes a coding region of interest (e.g., a nucleobase editing system or therapeutic polypeptide).

In some embodiments, a regulatory element may increase an amount of expression of a coding region of interest encoded on the circular RNA payload as compared to an amount expressed when no regulatory element exists.

In some embodiments, a regulatory element may comprise a sequence to selectively initiates or activates translation of a coding sequence of interest encoded on the circular RNA payload.

In some embodiments, a regulatory element may comprise a sequence to initiate degradation of the oRNA or the payload or cargo. Non-limiting examples of the sequence to initiate degradation includes, but is not limited to, riboswitch aptazyme and miRNA binding sites.

In some embodiments, a regulatory element can modulate translation of a coding region of interest encoded on the oRNA. The modulation can create an increase (enhancer) or decrease (suppressor) in the expression of the coding region of interest. The regulatory element may be located adjacent to the CROI (e.g., on one side or both sides of the CROI).

### Translation Initiation Sequence

In some embodiments, a translation initiation sequence functions as a regulatory element. In some embodiments, the translation initiation sequence comprises an AUG/ATG codon. In some embodiments, a translation initiation sequence comprises any eukaryotic start codon such as, but not limited to, AUG/ATG, CUG/CTG, GUG/GTG, UUG/TTG, ACG, AUC/ATC, AUU, AAG, AUA/ATA, or AGG. In some embodiments, a translation initiation sequence comprises a Kozak sequence. In some embodiments, translation begins at an alternative translation initiation sequence, e.g., translation initiation sequence other than AUG/ATG codon, under selective conditions, e.g., stress induced conditions. As a non-limiting example, the translation of the circular polyribonucleotide may begin at alternative translation initiation sequence, such as ACG. As another non-limiting example, the circular polyribonucleotide translation may begin at alternative translation initiation sequence, CUG/CTG. As another non-limiting example, the translation may begin at alternative translation initiation sequence, GUG/GTG. As yet another non-limiting example, the translation may begin at a repeat-associated non-AUG (RAN) sequence such as an alternative translation initiation sequence that includes short stretches of repetitive RNA e.g. CGG, GGGGCC, CAG, or CTG.

In some embodiments, the oRNA encodes a polypeptide or peptide and may comprise a translation initiation sequence. The translation initiation sequence may comprise, but is not limited to a start codon, a non-coding start codon, a Kozak sequence or a Shine-Dalgarno sequence. The translation initiation sequence may be located adjacent to the payload or cargo (e.g., on one side or both sides of the coding region of interest).

In some embodiments, the translation initiation sequence provides conformational flexibility to the oRNA. In some embodiments, the translation initiation sequence is within a substantially single stranded region of the oRNA.

The oRNA may include more than 1 start codon such as, but not limited to, at least 2, at least 3, at least 4, at least 5, at least 6, at least 7, at least 8, at least 9, at least 10, at least 11, at least 12, at least 13, at least 14, at least 15 or more than 15 start codons. Translation may initiate on the first start codon or may initiate downstream of the first start codon.

In some embodiments, the oRNA may initiate at a codon which is not the first start codon, e.g., AUG. Translation of the circular polyribonucleotide may initiate at an alternative translation initiation sequence, such as, but not limited to, ACG, AGG, AAG, CUG/CTG, GUG/GTG, AUA/ATA, AUU/ATT, UUG/TTG. In some embodiments, translation begins at an alternative translation initiation sequence under selective conditions, e.g., stress induced conditions. As a non-limiting example, the translation of the oRNA may begin at alternative translation initiation sequence, such as ACG. As another non-limiting example, the oRNA translation may begin at alternative translation initiation sequence, CUG/CTG. As yet another non-limiting example, the oRNA translation may begin at alternative translation initiation sequence, GTG/GUG. As yet another non-limiting example, the oRNA may begin translation at a repeat-associated non-AUG (RAN) sequence, such as an alternative translation initiation sequence that includes short stretches of repetitive RNA e.g. CGG, GGGGCC, CAG, CTG.

### IRES Sequences

In some embodiments, the oRNA described herein comprises an internal ribosome entry site (IRES) element capable of engaging an eukaryotic ribosome. In some embodiments, the TRES element is at least about 5 nucleotides, at least about 8 nucleotides, at least about 9 nucleotides, at least about 10 nucleotides, at least about 15 nucleotides, at least about 20 nucleotides, at least about 25 nucleotides, at least about 30 nucleotides, at least about 40 nucleotides, at least about 50 nucleotides, at least about 100 nucleotides, at least about 200 nucleotides, at least about 250 nucleotides, at least about 350 nucleotides, or at least about 500 nucleotides. In one embodiment, the IRES element is derived from the DNA of an organism including, but not limited to, a virus, a mammal, and a Drosophila. Such viral DNA may be derived from, but is not limited to, picornavirus complementary DNA (cDNA), with encephalomyocarditis virus (EMCV) cDNA and poliovirus cDNA. In one embodiment, Drosophila DNA from which an TIRES element is derived includes, but is not limited to, an Antennapedia gene from Drosophila melanogaster.

In some embodiments, the IRES element is at least partially derived from a virus, for instance, it can be derived from a viral IRES element, such as ABPV_IGRpred, AEV, ALPV IGRpred, BQCV IGRpred, BVDV1_1-385, BVDV1_29-391, CrPV_5NCR, CrPV_IGR, crTMV_REScp, crTMV_IRESmp75, crTMV_IRESmp228, crTMV_IREScp, crTMV_IREScp, CSFV, CVB3, DCV_IGR, EMCV-R, EoPV_5NTR, ERAV 245-961, ERBV 162-920, EV71_1-748, FeLV-Notch2, FMDV type_C, GBV-A, GBV-B, GBV-C, gypsy_env, gypsyD5, gypsyD2, HAV_HM175, HCV type_la, HiPV_IGRpred, HIV-1, HoCV1_IGRpred, HRV-2, IAPV_IGRpred, idefix, KBV_IGRpred, LINE-1_ORF1_-101_to_-1, LINE-1_ORF1-302_to_-202, LINE-1_ORF2-138_to_-86, LINE-1_ORF1_-44to_-1, PSIV_IGR, PV_typel_Mahoney, PV_type3_Leon, REV-A, RhPV_5NCR, RhPV_IGR, SINV1_IGRpred, SV40_661-830, TMEV, TMV_UI_IRESmp228, TRV_5NTR, TrV_IGR, or TSV_IGR. In some embodiments, the IRES element is at least partially derived from a cellular TRES, such as AML1/RUNX1, Antp-D, Antp-DE, Antp-CDE, Apaf-1, Apaf-1, AQP4, AT1R_var1, ATIR_var2, AT1R_var3, AT1R_var4, BAG1_p36delta236 nt, BAG1_p36, BCL2, BiP_-222_-3, c-IAP1_285-1399, c-IAP1_1313-1462, c-jun, c-myc, Cat-1224, CCND1, DAPS, eIF4G, eIF4GI-ext, eIF4GII, eIF4GII-long, ELG1, ELH, FGF1A, FMR1, Gtx-133-141, Gtx-1-166, Gtx-1-120, Gtx-1-196, hairless, HAP4, HIFla, hSNM1, Hspl01, hsp70, hsp70, Hsp90, IGF2_leader2, Kv1.4_1.2, L-myc, LamB1_-335_-1, LEF1, MNT_75-267, MNT_36-160, MTG8a, MYB, MYT2_997-1152, n-MYC, NDST1, NDST2, NDST3, NDST4L, NDST4S, NRF_-653_-17, NtHSF1, ODC1, p27kip1, 03_128-269, PDGF2/c-sis, Pim-1, PITSLRE_p58, Rbm3, reaper, Scamper, TFIID, TIF4631, Ubx_1-966, Ubx_373-961, UNR, Ure2, UtrA, VEGF-A-133-1, XIAP 5-464, XIAP 305-466, or YAP1.

In another embodiment, the TRES is an IRES sequence from Coxsackievirus B3 (CVB3), the protein coding region encodes Guassia luciferase (Gluc) and the spacer sequences are polyA-C.

In some embodiments, the TRES, if present, is at least about 50 nucleotides in length. In one embodiment, the vector comprises an IRES that comprises a natural sequence. In one embodiment, the vector comprises an IRES that comprises a synthetic sequence.

An IRES may act as the sole ribosome binding site, or may serve as one of multiple ribosome binding sites of an mRNA. A polynucleotide containing more than one functional ribosome binding site may encode several peptides or polypeptides that are translated independently by the ribosomes (e.g., multicistronic mRNA). When polynucleotides are provided with an IRES, further optionally provided is a second translatable region. Examples of IRES sequences that can be used according to the present disclosure include without limitation, those from picornaviruses (e.g., FMDV), pest viruses (CFFV), polio viruses (PV), encephalomyocarditis viruses (ECMV), foot-and mouth disease viruses (FMDV), hepatitis C viruses (HCV), classical Swine fever viruses (CSFV), murine leukemia virus (MLV), simian immune deficiency viruses (SIV) or cricket paralysis viruses (CrPV).

### Termination Element

In some embodiments, the oRNA includes one or more coding regions of interest (i.e., also referred to as product expression sequences) which encode polypeptides of interest, including but not limited to nucleobase editing system and therapeutic proteins. In various embodiments, the product expression sequences may or may not have a termination element.

In some embodiments, the oRNA includes one or more product expression sequences that lack a termination element, such that the oRNA is continuously translated.

Exclusion of a termination element may result in rolling circle translation or continuous expression of the encoded peptides or polypeptides as the ribosome will not stall or fall-off. In such an embodiment, rolling circle translation expresses continuously through the product expression sequence.

In some embodiments, one or more product expression sequences in the oRNA comprise a termination element.

In some embodiments, not all of the product expression sequences in the oRNA comprise a termination element. In such instances, the product expression sequence may fall off the ribosome when the ribosome encounters the termination element and terminates translation.

### Rolling Circle Translation

In some embodiments, once translation of the oRNA is initiated, the ribosome bound to the oRNA does not disengage from the oRNA before finishing at least one round of translation of the oRNA. In some embodiments, the oRNA as described herein is competent for rolling circle translation. In some embodiments, during rolling circle translation, once translation of the oRNA is initiated, the ribosome bound to the oRNA does not disengage from the oRNA before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 10⁵ rounds, or at least 10⁶ rounds of translation of the oRNA.

In some embodiments, the rolling circle translation of the oRNA leads to generation of polypeptide that is translated from more than one round of translation of the oRNA. In some embodiments, the oRNA comprises a stagger element, and rolling circle translation of the oRNA leads to generation of polypeptide product that is generated from a single round of translation or less than a single round of translation of the oRNA.

### Circularization

In one embodiment, a linear RNA may be cyclized, or concatemerized. In some embodiments, the linear RNA may be cyclized in vitro prior to formulation and/or delivery. In some embodiments, the linear RNA may be cyclized within a cell.

In some embodiments, the mechanism of cyclization or concatemerization may occur through at least 3 different routes: 1) chemical, 2) enzymatic, and 3) ribozyme catalyzed. The newly formed 5′-/3′-linkage may be intramolecular or intermolecular.

In the first route, the 5′-end and the 3′-end of the nucleic acid contain chemically reactive groups that, when close together, form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a synthetic mRNA molecule will undergo a nucleophilic attack on the 5′-NHS-ester moiety forming a new 5′-/3′-amide bond.

In the second route, T4 RNA ligase may be used to enzymatically link a 5′-phosphorylated nucleic acid molecule to the 3′-hydroxyl group of a nucleic acid forming a new phosphorodiester linkage. In an example reaction, \hat{ }g of a nucleic acid molecule is incubated at 37° C. for 1 hour with 1-10 units of T4 RNA ligase (New England Biolabs, Ipswich, MA) according to the manufacturer's protocol. The ligation reaction may occur in the presence of a split oligonucleotide capable of base-pairing with both the 5′- and 3′-region in juxtaposition to assist the enzymatic ligation reaction.

In the third route, either the 5′- or 3′-end of the cDNA template encodes a ligase ribozyme sequence such that during in vitro transcription, the resultant nucleic acid molecule can contain an active ribozyme sequence capable of ligating the 5′-end of a nucleic acid molecule to the 3′-end of a nucleic acid molecule. The ligase ribozyme may be derived from the Group I Intron, Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, the oRNA is made via circularization of a linear RNA.

In some embodiments, the following elements are operably connected to each other and, in some embodiments, arranged in the following sequence: a) a 5′ homology arm, b) a 3′ group I intron fragment containing a 3′ splice site dinucleotide, c) a protein coding or noncoding region, d) a 5′ group I intron fragment containing a 5′ splice site dinucleotide, and e) a 3′ homology arm. In certain embodiments said vector allows production of a circular RNA that is translatable and/or biologically active inside eukaryotic cells. In some embodiments, the biologically active RNA is, for example, an miRNA sponge, or long noncoding RNA.

In some embodiments, the following elements are operably connected to each other and arranged in the following sequence: a) a 5′ homology arm, b) a 3′ group I intron fragment containing a 3′ splice site dinucleotide, c) optionally, a 5′ spacer sequence, d) optionally, an internal ribosome entry site (TRES), e) a protein coding or noncoding region, f) optionally, a 3′ spacer sequence, g) a 5′ group I intron fragment containing a 5′ splice site dinucleotide, and h) a 3′ homology arm. In certain embodiments, said vector allows production of a circular RNA that is translatable and/or biologically active inside eukaryotic cells.

In some embodiments, the following elements are operably connected to each other and arranged in the following sequence: a) a 5′ homology arm, b) a 3′ group I intron fragment containing a 3′ splice site dinucleotide, c) a 5′ spacer sequence, d) an internal ribosome entry site (IRES), e) a protein coding or noncoding region, f) a 5′ group I intron fragment containing a 5′ splice site dinucleotide, and g) a 3′ homology arm. In some embodiments, said vector allows production of a circular RNA that is translatable and/or biologically active inside eukaryotic cells.

In some embodiments, the following elements are operably connected to each other and arranged in the following sequence: a) a 5′ homology arm, b) a 3′ group I intron fragment containing a 3′ splice site dinucleotide, c) a 5′ spacer sequence, d) a protein coding or noncoding region, e) a 3′ spacer sequence, f) a 5′ group I intron fragment containing a 5′ splice site dinucleotide, and g) a 3′ homology arm. In some embodiments, said vector allows production of a circular RNA that is translatable and/or biologically active inside eukaryotic cells.

In some embodiments, the following elements are operably connected to each other and arranged in the following sequence: a) a 5′ homology arm, b) a 3′ group I intron fragment containing a 3′ splice site dinucleotide, c) an internal ribosome entry site (IRES), d) a protein coding or noncoding region, e) a 3′ spacer sequence, f) a 5′ group I intron fragment containing a 5′ splice site dinucleotide, and g) a 3′ homology arm. In some embodiments, said vector allows production of a circular RNA that is translatable and/or biologically active inside eukaryotic cells.

In some embodiments, the following elements are operably connected to each other and arranged in the following sequence: a) a 5′ homology arm, b) a 3′ group I intron fragment containing a 3′ splice site dinucleotide, c) a protein coding or noncoding region, d) a 3′ spacer sequence, e) a 5′ group I intron fragment containing a 5′ splice site dinucleotide, and f) a 3′ homology arm. In some embodiments, said vector allows production of a circular RNA that is translatable and/or biologically active inside eukaryotic cells.

In some embodiments, the following elements are operably connected to each other and arranged in the following sequence: a) a 5′ homology arm, b) a 3′ group I intron fragment containing a 3′ splice site dinucleotide, c) a 5′ spacer sequence, d) a protein coding or noncoding region, e) a 5′ group I intron fragment containing a 5′ splice site dinucleotide, and f) a 3′ homology arm. In some embodiments, said vector allows production of a circular RNA that is translatable and/or biologically active inside eukaryotic cells.

In some embodiments, the following elements are operably connected to each other and arranged in the following sequence: a) a 5′ homology arm, b) a 3′ group I intron fragment containing a 3′ splice site dinucleotide, c) an internal ribosome entry site (IRES), d) a protein coding or noncoding region, e) a 5′ group I intron fragment containing a 5′ splice site dinucleotide, and f) a 3′ homology arm. In some embodiments, said vector allows production of a circular RNA that is translatable and/or biologically active inside eukaryotic cells.

In some embodiments, the following elements are operably connected to each other and arranged in the following sequence: a) a 5′ homology arm, b) a 3′ group I intron fragment containing a 3′ splice site dinucleotide, c) a 5′ spacer sequence, d) an internal ribosome entry site (IRES), e) a protein coding or noncoding region, f) a 3′ spacer sequence, g) a 5′ group I intron fragment containing a 5′ splice site dinucleotide, and h) a 3′ homology arm. In some embodiments, said vector allowing production of a circular RNA that is translatable and/or biologically active inside eukaryotic cells.

In one embodiment, the 3′ group I intron fragment and/or the 5′ group I intron fragment is from a Cyanobacterium Anabaena sp. pre-tRNA-Leu gene or T4 phage Td gene.

In one embodiment, the 3′ group I intron fragment and/or the 5′ group I intron fragment is from a Cyanobacterium Anabaena sp. pre-tRNA-Leu gene.

In one embodiment, the protein coding region encodes a protein of eukaryotic or prokaryotic origin. In another embodiment, the protein coding region encodes human protein or non-human protein. In some embodiments, the protein coding region encodes one or more antibodies. For example, in some embodiments, the protein coding region encodes human antibodies. In one embodiment, the protein coding region encodes a protein selected from hFIX, SP-B, VEGF-A, human methylmalonyl-CoA mutase (hMVUT), CFTR, cancer self-antigens, and additional gene editing enzymes like Cpf1, zinc finger nucleases (ZFNs) and transcription activator-like effector nucleases (TALENs). In another embodiment, the protein coding region encodes a protein for therapeutic use. In one embodiment, the human antibody encoded by the protein coding region is an anti-HIV antibody. In one embodiment, the antibody encoded by the protein coding region is a bispecific antibody. In one embodiment, the bispecific antibody is specific for CD19 and CD22. In another embodiment, the bispecific antibody is specific for CD3 and CLDN6. In one embodiment, the protein coding region encodes a protein for diagnostic use. In one embodiment, the protein coding region encodes Gaussia luciferase (Gluc), Firefly luciferase (Fluc), enhanced green fluorescent protein (eGFP), human erythropoietin (hEPO), or Cas9 endonuclease.

In one embodiment, the 5′ homology arm is about 5-50 nucleotides in length. In another embodiment, the 5′ homology arm is about 9-19 nucleotides in length. In some embodiments, the 5′ homology arm is at least 5, 6, 7, 8, 9, 10, 11, 12, 13, 14, 15, 16, 17, 18 or 19 nucleotides in length. In some embodiments, the 5′ homology arm is no more than 50, 45, 40, 35, 30, 25 or 20 nucleotides in length. In some embodiments, the 5′ homology arm is 5, 6, 7, 8, 9, 10, 11, 12, 13, 14, 15, 16, 17, 18 or 19 nucleotides in length.

In one embodiment, the 3′ homology arm is about 5-50 nucleotides in length. In another embodiment, the 3′ homology arm is about 9-19 nucleotides in length. In some embodiments, the 3′ homology arm is at least 5, 6, 7, 8, 9, 10, 11, 12, 13, 14, 15, 16, 17, 18 or 19 nucleotides in length. In some embodiments, the 3′ homology arm is no more than 50, 45, 40, 35, 30, 25 or 20 nucleotides in length. In some embodiments, the 3′ homology arm is 5, 6, 7, 8, 9, 10, 11, 12, 13, 14, 15, 16, 17, 18 or 19 nucleotides in length.

In one embodiment, the 5′ spacer sequence is at least 10 nucleotides in length. In another embodiment, the 5′ spacer sequence is at least 15 nucleotides in length. In a further embodiment, the 5′ spacer sequence is at least 30 nucleotides in length. In some embodiments, the 5′ spacer sequence is at least 7, 8, 9, 10, 11, 12, 13, 14, 15, 16, 17, 18, 19, 20, 25 or 30 nucleotides in length. In some embodiments, the 5′ spacer sequence is no more than 100, 90, 80, 70, 60, 50, 45, 40, 35 or 30 nucleotides in length. In some embodiments the 5′ spacer sequence is between 20 and 50 nucleotides in length. In certain embodiments, the 5′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 5′ spacer sequence is a polyA sequence. In another embodiment, the 5′ spacer sequence is a polyA-C sequence.

In one embodiment, the 3′ spacer sequence is at least 10 nucleotides in length. In another embodiment, the 3′ spacer sequence is at least 15 nucleotides in length. In a further embodiment, the 3′ spacer sequence is at least 30 nucleotides in length. In some embodiments, the 3′ spacer sequence is at least 7, 8, 9, 10, 11, 12, 13, 14, 15, 16, 17, 18, 19, 20, 25 or 30 nucleotides in length. In some embodiments, the 3′ spacer sequence is no more than 100, 90, 80, 70, 60, 50, 45, 40, 35 or 30 nucleotides in length. In some embodiments the 3′ spacer sequence is between 20 and 50 nucleotides in length. In certain embodiments, the 3′ spacer sequence is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length. In one embodiment, the 3′ spacer sequence is a polyA sequence. In another embodiment, the 5′ spacer sequence is a polyA-C sequence.

### Extracellular Circularization

In some embodiments, the linear RNA is cyclized, or concatemerized using a chemical method to form an oRNA. In some chemical methods, the 5′-end and the 3′-end of the nucleic acid (e.g., a linear RNA) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA will undergo a nucleophilic attack on the 5′-NHS-ester moiety forming a new 5′-/3′-amide bond.

In one embodiment, a DNA or RNA ligase may be used to enzymatically link a 5′-phosphorylated nucleic acid molecule (e.g., a linear RNA) to the 3′-hydroxyl group of a nucleic acid (e.g., a linear nucleic acid) forming a new phosphorodiester linkage. In an example reaction, a linear RNA is incubated at 37C for 1 hour with 1-10 units of T4 RNA ligase according to the manufacturer's protocol. The ligation reaction may occur in the presence of a linear nucleic acid capable of base-pairing with both the 5′- and 3′-region in juxtaposition to assist the enzymatic ligation reaction. In one embodiment, the ligation is splint ligation where a single stranded polynucleotide (splint), like a single stranded RNA, can be designed to hybridize with both termini of a linear RNA, so that the two termini can be juxtaposed upon hybridization with the single-stranded splint. Splint ligase can thus catalyze the ligation of the juxtaposed two termini of the linear RNA, generating an oRNA.

In one embodiment, a DNA or RNA ligase may be used in the synthesis of the oRNA. As a non-limiting example, the ligase may be a circ ligase or circular ligase.

In one embodiment, either the 5′- or 3′-end of the linear RNA can encode a ligase ribozyme sequence such that during in vitro transcription, the resultant linear RNA includes an active ribozyme sequence capable of ligating the 5′-end of the linear RNA to the 3′-end of the linear RNA. The ligase ribozyme may be derived from the Group I Intron, Hepatitis Delta Virus, Hairpin ribozyme or may be selected by SELEX (systematic evolution of ligands by exponential enrichment).

In one embodiment, a linear RNA may be cyclized or concatemerized by using at least one non-nucleic acid moiety. In one aspect, the at least one non-nucleic acid moiety may react with regions or features near the 5′ terminus and/or near the 3′ terminus of the linear RNA in order to cyclize or concatermerize the linear RNA. In another aspect, the at least one non-nucleic acid moiety may be located in or linked to or near the 5′ terminus and/or the 3′ terminus of the linear RNA. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In one embodiment, a linear RNA may be cyclized or concatemerized due to a non-nucleic acid moiety that causes an attraction between atoms, molecular surfaces at, near or linked to the 5′ and 3′ ends of the linear RNA. As a non-limiting example, one or more linear RNA may be cyclized or concatemerized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In one embodiment, the linear RNA may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear RNA to cyclize or concatemerize. In another aspect, the peptides covalently linked to the ribozyme RNA near the 5′ terminus and the 3′ terminus may cause the linear RNA to cyclize or concatemerize after being subjected to ligated using various methods known in the art such as, but not limited to, protein ligation.

In some embodiments, the linear RNA may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear RNA into a 5′ monophosphate may occur by a two-step reaction comprising: (a) contacting the 5′ nucleotide of the linear RNA with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, RNA may be circularized using the methods described in WO2017222911 and WO2016197121, the contents of each of which are herein incorporated by reference in their entirety.

In some embodiments, RNA may be circularized, for example, by back splicing of a non-mammalian exogenous intron or splint ligation of the 5′ and 3′ends of a linear RNA. In one embodiment, the circular RNA is produced from a recombinant nucleic acid encoding the target RNA to be made circular. As a non-limiting example, the method comprises: a) producing a recombinant nucleic acid encoding the target RNA to be made circular, wherein the recombinant nucleic acid comprises in 5′ to 3′order: i) a 3′portion of an exogenous intron comprising a 3′ splice site, ii) a nucleic acid sequence encoding the target RNA, and iii) a 5′ portion of an exogenous intron comprising a 5′ splice site; b) performing transcription, whereby RNA is produced from the recombinant nucleic acid; and c) performing splicing of the RNA, whereby the RNA circularizes to produce a oRNA.

While not wishing to be bound by theory, circular RNAs generated with exogenous introns are recognized by the immune system as “non-self” and trigger an innate immune response. On the other hand, circular RNAs generated with endogenous introns are recognized by the immune system as “self” and generally do not provoke an innate immune response, even if carrying an exon comprising foreign RNA.

Accordingly, circular RNAs can be generated with either an endogenous or exogenous intron to control immunological self/non-self discrimination as desired. Numerous intron sequences from a wide variety of organisms and viruses are known and include sequences derived from genes encoding proteins, ribosomal RNA (rRNA), or transfer RNA (tRNA).

Circular RNAs can be produced from linear RNAs in a number of ways. In some embodiments, circular RNAs are produced from a linear RNA by backsplicing of a downstream 5′ splice site (splice donor) to an upstream 3′ splice site (splice acceptor). Circular RNAs can be generated in this manner by any nonmammalian splicing method. For example, linear RNAs containing various types of introns, including self-splicing group I introns, self-splicing group II introns, spliceosomal introns, and tRNA introns can be circularized. In particular, group I and group II introns have the advantage that they can be readily used for production of circular RNAs in vitro as well as in vivo because of their ability to undergo self-splicing due to their autocatalytic ribozyme activity.

In some embodiments, circular RNAs can be produced in vitro from a linear RNA by chemical or enzymatic ligation of the 5′ and 3′ ends of the RNA. Chemical ligation can be performed, for example, using cyanogen bromide (BrCN) or ethyl-3-(3′-dimethylaminopropyl) carbodiimide (EDC) for activation of a nucleotide phosphomonoester group to allow phosphodiester bond formation. See e.g., Sokolova (1988) FEBS Lett 232: 153-155; Dolinnaya et al. (1991) Nucleic Acids Res., 19:3067-3072; Fedorova (1996) Nucleosides Nucleotides Nucleic Acids 15: 1 137-1 147; herein incorporated by reference. Alternatively, enzymatic ligation can be used to circularize RNA. Exemplary ligases that can be used include T4 DNA ligase (T4 Dnl), T4 RNA ligase 1 (T4 Rnl 1), and T4 RNA ligase 2 (T4 Rnl 2).

In some embodiments, splint ligation using an oligonucleotide splint that hybridizes with the two ends of a linear RNA can be used to bring the ends of the linear RNA together for ligation. Hybridization of the splint, which can be either a DNA or a RNA, orientates the 5′-phosphate and 3′-OH of the RNA ends for ligation. Subsequent ligation can be performed using either chemical or enzymatic techniques, as described above. Enzymatic ligation can be performed, for example, with T4 DNA ligase (DNA splint required), T4 RNA ligase 1 (RNA splint required) or T4 RNA ligase 2 (DNA or RNA splint). Chemical ligation, such as with BrCN or EDC, in some cases is more efficient than enzymatic ligation if the structure of the hybridized splint-RNA complex interferes with enzymatic activity.

In some embodiments, the oRNA may further comprise an internal ribosome entry site (IRES) operably linked to an RNA sequence encoding a polypeptide. Inclusion of an IRES permits the translation of one or more open reading frames from a circular RNA. The IRES element attracts a eukaryotic ribosomal translation initiation complex and promotes translation initiation. See, e.g., Kaufman et al., Nuc. Acids Res. (1991) 19:4485-4490; Gurtu et al., Biochem. Biophys. Res. Comm. (1996) 229:295-298; Rees et al., BioTechniques (1996) 20: 102-110; Kobayashi et al., BioTechniques (1996) 21:399-402; and Mosser et al., BioTechniques 1997 22 150-161).

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

### Splicing Element

In some embodiments, the oRNA includes at least one splicing element. The splicing element can be a complete splicing element that can mediate splicing of the oRNA or the spicing element can be a residual splicing element from a completed splicing event. For instance, in some cases, a splicing element of a linear RNA can mediate a splicing event that results in circularization of the linear RNA, thereby the resultant oRNA comprises a residual splicing element from such splicing-mediated circularization event. In some cases, the residual splicing element is not able to mediate any splicing. In other cases, the residual splicing element can still mediate splicing under certain circumstances. In some embodiments, the splicing element is adjacent to at least one expression sequence. In some embodiments, the oRNA includes a splicing element adjacent each expression sequence. In some embodiments, the splicing element is on one or both sides of each expression sequence, leading to separation of the expression products, e.g., peptide(s) and or polypeptide(s).

In some embodiments, the oRNA includes an internal splicing element that when replicated the spliced ends are joined together. Some examples may include miniature introns (<100 nt) with splice site sequences and short inverted repeats (30-40 nt) such as AluSq2, AluJr, and AluSz, inverted sequences in flanking introns, Alu elements in flanking introns, and motifs found in (suptable4 enriched motifs) cis-sequence elements proximal to backsplice events such as sequences in the 200 bp preceding (upstream of) or following (downstream from) a backsplice site with flanking exons. In some embodiments, the oRNA includes at least one repetitive nucleotide sequence described elsewhere herein as an internal splicing element. In such embodiments, the repetitive nucleotide sequence may include repeated sequences from the Alu family of introns. See, e.g., U.S. Pat. No. 11,058,706.

In some embodiments, the oRNA may include canonical splice sites that flank head-to-tail junctions of the oRNA.

In some embodiments, the oRNA may include a bulge-helix-bulge motif, comprising a 4-base pair stem flanked by two 3-nucleotide bulges. Cleavage occurs at a site in the bulge region, generating characteristic fragments with terminal 5′-hydroxyl group and 2′,3′-cyclic phosphate. Circularization proceeds by nucleophilic attack of the 5′-OH group onto the 2′, 3′-cyclic phosphate of the same molecule forming a 3′, 5′-phosphodiester bridge.

In some embodiments, the oRNA may include a sequence that mediates self-ligation. Non-limiting examples of sequences that can mediate self-ligation include a self-circularizing intron, e.g., a 5′ and 3′ slice junction, or a self-circularizing catalytic intron such as a Group I, Group II or Group III Introns. Non-limiting examples of group I intron self-splicing sequences may include self-splicing permuted intron-exon sequences derived from T4 bacteriophage gene td, and the intervening sequence (IVS) rRNA of Tetrahymena.

### Other Circularization Methods

In some embodiments, linear RNA may include complementary sequences, including either repetitive or nonrepetitive nucleic acid sequences within individual introns or across flanking introns. In some embodiments, the oRNA includes a repetitive nucleic acid sequence. In some embodiments, the repetitive nucleotide sequence includes poly CA or poly UG sequences. In some embodiments, the oRNA includes at least one repetitive nucleic acid sequence that hybridizes to a complementary repetitive nucleic acid sequence in another segment of the oRNA, with the hybridized segment forming an internal double strand. In some embodiments, repetitive nucleic acid sequences and complementary repetitive nucleic acid sequences from two separate oRNA that hybridize to generate a single oRNA, with the hybridized segments forming internal double strands. In some embodiments, the complementary sequences are found at the 5′ and 3′ ends of the linear RNA. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the oRNA. Such methods may include, but are not limited to click chemistry (e.g., alkyne- and azide-based methods, or clickable bases), olefin metathesis, phosphoramidate ligation, hemiaminal-imine crosslinking, base modification, and any combination thereof. In some embodiments, enzymatic methods of circularization may be used to generate the oRNA. In some embodiments, a ligation enzyme, e.g., DNA or RNA ligase, may be used to generate a template of the oRNA or complement, a complementary strand of the oRNA, or the oRNA.

Any of the circular polynucleotides as taught in for example U.S. Provisional Application No. 61/873,010 filed Sep. 3, 2013 or U.S. Pat. No. 10,709,779, may be used herein. The contents of these references are incorporated herein by reference in their entirety. In addition, any of the circular RNAs, methods for making circular RNAs, circular RNA compositions that are described in the following publications are contemplated herein and are incorporated by reference in their entireties are part of the instant specification: U.S. Pat. Nos. 11,352,640, 11,352,641, 11,203,767, 10,683,498, 5,773,244, and 5,766,903; US Application Publications US 2022/0177540, US 2021/0371494, US 2022/0090137, US 2019/0345503, and US 2015/0299702; and PCT Application Publications WO 2021/226597, WO 2019/236673, WO 2017/222911, WO2016/187583, WO2014/082644 and WO 1997/007825.

### K. KITS

Also provided are kits comprising engineered retrons (e.g., engineered nucleic acid constructs, or engineered nucleic acid-enzyme constructs) as described herein.

In some embodiments, the kit provides an engineered retron construct or a vector system comprising such a retron construct. In some embodiments, the engineered retron construct, included in the kit, comprises a heterologous sequence capable of providing a cell with a nucleic acid encoding a protein or regulatory RNA of interest, a cellular barcode, a donor polynucleotide suitable for use in gene editing, e.g., by homology directed repair (HDR) or recombination-mediated genetic engineering (recombineering), or a CRISPR protospacer DNA sequence for use in molecular recording. Other agents may also be included in the kit such as transfection agents, host cells, suitable media for culturing cells, buffers, and the like.

In the context of a kit, agents can be provided in liquid or solid form in any convenient packaging (e.g., stick pack, dose pack, etc.). The agents of a kit can be present in the same or separate containers. The kits may contain any one or more of the components described herein in one or more containers. The components may be prepared sterilely, packaged in a syringe and shipped refrigerated. Alternatively it may be housed in a vial or other container for storage. A second container may have other components prepared sterilely. Alternatively the kits may include the active agents premixed and shipped in a vial, tube, or other container.

The kits may have a variety of forms, such as a blister pouch, a shrink wrapped pouch, a vacuum sealable pouch, a sealable thermoformed tray, or a similar pouch or tray form, with the accessories loosely packed within the pouch, one or more tubes, containers, a box or a bag. The kits may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kits may also include other components, depending on the specific application, for example, containers, cell media, salts, buffers, reagents, syringes, needles, a fabric, such as gauze, for applying or removing a disinfecting agent, disposable gloves, a support for the agents prior to administration, etc. Some aspects of this disclosure provide kits comprising a nucleic acid construct comprising a nucleotide sequence encoding the various components of the retron-based editing system described herein.

In addition to the above components, the subject kits may further include (in some embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a remote site. In some embodiments, information provided on the website is periodically updated to provide, for example, the most up-to-date information. The written instructions may be in a form prescribed by a governmental agency regulating the manufacture, use, or sale of pharmaceuticals or biological products, which can also reflect approval by the agency of manufacture, use or sale for animal administration. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the disclosure.

Other aspects of this disclosure provide kits comprising one or more nucleic acid constructs (e.g., one or more mRNA or circular RNA molecules encoding the components of the retron-based genome editing system) In various embodiments, all nucleic acid constructs can be based on RNA molecules, i.e., and “all-RNA system.” For example, each of the components of the editing system could be expressed from a mRNA molecule, which would be delivered to a target cell by one or more delivery methods (e.g., LNP delivery).

### L. CELLS

One aspect of the disclosure provides an isolated host cell that includes one or more of the compositions described herein, including, but not limited to, engineered retrons and/or retron components, engineered ncRNAs, engineered msDNA, engineered RT, nucleic acid molecules encoding the engineered retrons and/or retron components, and vector or vector systems encoding the engineered retrons and/or retron components, and any combinations thereof. In some embodiments, the host cell is a prokaryotic cell, an archaeal cell, or a eukaryotic host cell. In some embodiments, the eukaryotic host cell is a mammalian cell, such as a human cell, a non-human cell, or a non-human mammalian cell. In some embodiments, the host cell is an artificial cell or genetically modified cell. In some embodiments, the host cell is in vitro, such as a tissue culture cell. In some embodiments, the host cell is within a living host organism.

Cells that may contain any of the compositions described herein. The methods described herein are used to deliver recombinant retrons or components thereof into a eukaryotic cell (e.g., a mammalian cell, such as a human cell). In some embodiments, the cell is in vitro (e.g., cultured cell. In some embodiments, the cell is in vivo (e.g., in a subject such as a human subject). In some embodiments, the cell is ex vivo (e.g., isolated from a subject and may be administered back to the same or a different subject).

The present disclosure contemplates the use of any suitable host cell. For example, the cell host can be a mammalian cell. Mammalian cells of the present disclosure include human cells, primate cells (e.g., vero cells), rat cells (e.g., GH3 cells, OC23 cells) or mouse cells (e.g., MC3T3 cells). There are a variety of human cell lines, including, without limitation, human embryonic kidney (HEK) cells, HeLa cells, cancer cells from the National Cancer Institute's 60 cancer cell lines (NCI60), DU145 (prostate cancer) cells, Lncap (prostate cancer) cells, MCF-7 (breast cancer) cells, MDA-MB-438 (breast cancer) cells, PC3 (prostate cancer) cells, T47D (breast cancer) cells, THP-1 (acute myeloid leukemia) cells, U87 (glioblastoma) cells, SHSY5Y human neuroblastoma cells (cloned from a myeloma) and Saos-2 (bone cancer) cells. In some embodiments, the cells can be human embryonic kidney (HEK) cells (e.g., HEK 293 or HEK 293T cells). In some embodiments, the cells can be stem cells (e.g., human stem cells) such as, for example, pluripotent stem cells (e.g., human pluripotent stem cells including human induced pluripotent stem cells (hiPSCs)). A stem cell refers to a cell with the ability to divide for indefinite periods in culture and to give rise to specialized cells. A pluripotent stem cell refers to a type of stem cell that is capable of differentiating into all tissues of an organism, but not alone capable of sustaining full organismal development. A human induced pluripotent stem cell refers to a somatic (e.g., mature or adult) cell that has been reprogrammed to an embryonic stem cell-like state by being forced to express genes and factors important for maintaining the defining properties of embryonic stem cells (see, e.g., Takahashi and Yamanaka, Cell 126 (4): 663-76, 2006, incorporated by reference herein). Human induced pluripotent stem cells express stem cell markers and are capable of generating cells characteristic of all three germ layers (ectoderm, endoderm, mesoderm).

Some aspects of this disclosure provide cells comprising any of the compositions disclosed herein, including, but not limited to, engineered retrons and/or retron components, engineered ncRNAs, engineered msDNA, engineered RT, nucleic acid molecules encoding the engineered retrons and/or retron components, and vector or vector systems encoding the engineered retrons and/or retron components, and any combinations thereof. In some embodiments, a host cell is transiently or non-transiently transfected with one or more delivery systems described herein, including virus-based systems, virus-like particle systems, and non-virus-base delivery, including LNPs and liposomes. In some embodiments, a cell is transfected as it naturally occurs in a subject. In some embodiments, a cell that is transfected is taken from a subject, i.e., ex vivo transfection. In some embodiments, the cell is derived from cells taken from a subject, such as a cell line. A wide variety of cell lines for tissue culture are known in the art. Examples of cell lines include, but are not limited to, C8161, CCRF-CEM, MOLT, mTMCD-3, NHDF, HeLa—S3, Huh1, Huh4, Huh7, HUVEC, HASMC, HEKn, HEKa, MiaPaCell, Panc1, PC-3, TFI, CTLL-2, C1R, Rat6, CV1, RPTE, A10, T24, J82, A375, ARH-77, Calul, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS—C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO—IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, H1B55, HCA2, HEK-293, HeLa, Hepalclc7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI—H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

Cell lines are available from a variety of sources known to those with skill in the art (see, e.g., the American Type Culture Collection (ATCC) (Manassas, Va.)). In some embodiments, a cell transfected with one or more retron delivery systems described herein is used to establish a new cell line comprising one or more nucleic acid molecules encoding the recombinant retron-based gene editing systems described herein, or encoding at last a component of said systems (e.g., a recombinant ncRNA or a recombinant retron RT).

### M. PHARMACEUTICAL COMPOSITIONS

The engineered retron-based genome editing systems described herein, or one or more components thereof (e.g., engineered ncRNAs, engineered msDNA, engineered RT, nucleic acid molecules encoding the engineered retrons and/or retron components, guide RNAs, programmable nucleases) may be provided as pharmaceutical compositions. For example, one or more LNPs or other non-virus-based delivery system comprising one or more circular or linear RNA molecules encoding each of the components of the retron-based genome editing system may be formulated as a pharmaceutical composition for administering to a subject in need (e.g., a human in need of gene editing).

Formulations can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients. As used herein, the phrase “active ingredient” generally refers an engineered retron as described herein.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Other aspects of the present disclosure relate to pharmaceutical compositions comprising any of the various components of the recombinant retron-based genome editing systems described herein, including, but not limited to, engineered retrons and/or retron components, engineered ncRNAs, engineered msDNA, engineered RT, nucleic acid molecules encoding the engineered retrons and/or retron components, programmable nucleases (e.g., RNA-guided nucleases), guide RNAs, and vector or vector systems encoding the engineered retrons and/or retron components, and any combinations thereof. The term“pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g. for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is“acceptable” in the sense of being compatible with the other ingredients of the formulation and not injurious to the tissue of the subject (e.g., physiologically compatible, sterile, physiologic pH, etc.).

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as“excipient”, “carrier”, “pharmaceutically acceptable carrier” or the like are used interchangeably herein.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., tumor site). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105). Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal or LNP, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

Further, the pharmaceutical composition can be provided as a pharmaceutical kit comprising (a) a container containing a recombinant retron-based genome editing system or one or more components thereof in lyophilized form and (b) a second container containing a pharmaceutically acceptable diluent (e.g., sterile water) for injection. The pharmaceutically acceptable diluent can be used for reconstitution or dilution of the lyophilized system of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierce-able by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### N. USE AND METHODS OF USE

The engineered retron comprising the heterologous nucleic acid sequence can be used in a variety of applications, several non-limiting examples of which are described herein. In general, the engineered retron can be used in any suitable organism. In some embodiments, the organism is a eukaryote.

In some embodiments, the organism is an animal. In some embodiments, the animal is a fish, an amphibian, a reptile, a mammal, or a bird. In some embodiments, the animal is a farm animal or agriculture animal. Non-limiting examples of farm and agriculture animals include horses, goats, sheep, swine, cattle, llamas, alpacas, and birds, e.g., chickens, turkeys, ducks, and geese. In some embodiments, the animal is a non-human primate, e.g., baboons, capuchin monkeys, chimpanzees, lemurs, macaques, marmosets, tamarins, spider monkeys, squirrel monkeys, and vervet monkeys. In some embodiments, the animal is a pet. Non-limiting examples of pets include dogs, cats horses, wolfs, rabbits, ferrets, gerbils, hamsters, chinchillas, fancy rats, guinea pigs, canaries, parakeets, and parrots.

In some embodiments, the organism is a plant. Plants that may be transfected with an engineered retron include monocots and dicots. Particular examples include, but are not limited to, corn (maize), sorghum, wheat, sunflower, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers. Vegetables include, but are not limited to, crucifers, peppers, tomatoes, lettuce, green beans, lima beans, peas, and members of the genus Cucumis such as cucumber, cantaloupe, and musk melon. Ornamentals include, but are not limited to, azalea, hydrangea, hibiscus, roses, tulips, daffodils, petunias, carnation, poinsettia, and chrysanthemum.

In some embodiments, heterologous nucleic acid sequences can be added to the subject engineered retron to provide a cell with a heterologous nucleic acid encoding a protein or regulatory RNA of interest, a cellular barcode, a donor polynucleotide suitable for use in gene editing, e.g., by homology directed repair (HDR) or recombination-mediated genetic engineering (recombineering), or a CRISPR protospacer DNA sequence for use in molecular recording, as discussed further below. Such heterologous sequences may be inserted, for example, into the msr locus or the msd locus such that the heterologous sequence is transcribed by the retron reverse transcriptase as part of the msDNA product.

In some embodiments, the engineered retrons described herein may be used for research tools, such as kits, functional genomics assays, and generating engineered cell lines and animal models for research and drug screening. The kit may comprise one or more reagents in addition to the engineered retron, such as a buffer, a control reagent, a control vector, a control RNA polynucleotide, a reagent for in vitro production of the polypeptide from DNA, and adaptors for sequencing. A buffer can be, for example, a stabilization buffer, a reconstituting buffer, a diluting buffer, a wash buffer, or a buffer for introducing a polypeptide and/or polynucleotide of the kit into a cell. In some instances, a kit can comprise one or more additional reagents specific for plants. One or more additional reagents for plants can include, for example, soil, nutrients, plants, seeds, spores, Agrobacterium, a T-DNA vector, and a pBINAR vector.

### Gene Editing

In some embodiments, the retron is used for genome editing a desired site. A retron is engineered with a heterologous nucleic acid sequence encoding a donor polynucleotide suitable for use with nuclease genome editing system. The nuclease is designed to specifically target a location proximal to the desired edit (the nuclease should be designed such that it will not cut the target once the edit is properly installed). The nuclease (e.g., CAS or non-CAS) is linked to the retron, either by direct fusion to the RT or by fusion of the msDNA to the gRNA (only applicable for RNA-guided nucleases). A heterologous nucleic acid sequence is inserted into the retron msd. See for example FIG. 3, which shows a marker representing the edit.

In some embodiments, the heterologous nucleic acid sequence has 10-100 or more bp of homologous nucleic acid sequence to the genome on both sides of the desired edit. The desired edit (insertion, deletion, or mutation) is in between the homologous sequence.

In some embodiments, donor polynucleotides comprise a sequence comprising an intended genome edit flanked by a pair of homology arms responsible for targeting the donor polynucleotide to the target locus to be edited in a cell. The donor polynucleotide typically comprises a 5′ homology arm that hybridizes to a 5′ genomic target sequence and a 3′ homology arm that hybridizes to a 3′ genomic target sequence. The homology arms are referred to herein as 5′ and 3′ (i.e., upstream and downstream) homology arms, which relate to the relative position of the homology arms to the nucleotide sequence comprising the intended edit within the donor polynucleotide. The 5′ and 3′ homology arms hybridize to regions within the target locus in the genomic DNA to be modified, which are referred to herein as the “5′ target sequence” and “3′ target sequence,” respectively.

The homology arm must be sufficiently complementary for hybridization to the target sequence to mediate homologous recombination between the donor polynucleotide and genomic DNA at the target locus. For example, a homology arm may comprise a nucleotide sequence having at least about 80-100% sequence identity to the corresponding genomic target sequence, including any percent identity within this range, such as at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity thereto, wherein the nucleotide sequence comprising the intended edit can be integrated into the genomic DNA by HDR at the genomic target locus recognized (i.e., having sufficient complementary for hybridization) by the 5′ and 3′ homology arms.

In some embodiments, the corresponding homologous nucleotide sequences in the genomic target sequence (i.e., the “5′ target sequence” and “3′ target sequence”) flank a specific site for cleavage and/or a specific site for introducing the intended edit. The distance between the specific cleavage site and the homologous nucleotide sequences (e.g., each homology arm) can be several hundred nucleotides. In some embodiments, the distance between a homology arm and the cleavage site is 200 nucleotides or less (e.g., 0, 10, 20, 30, 50, 75, 100, 125, 150, 175, and 200 nucleotides). In most cases, a smaller distance may give rise to a higher gene targeting rate. In some embodiments, the donor polynucleotide is substantially identical to the target genomic sequence, across its entire length except for the sequence changes to be introduced to a portion of the genome that encompasses both the specific cleavage site and the portions of the genomic target sequence to be altered.

A homology arm can be of any length, e.g. 10 nucleotides or more, 15 nucleotides or more, 20 nucleotides or more, 50 nucleotides or more, 100 nucleotides or more, 250 nucleotides or more, 300 nucleotides or more, 350 nucleotides or more, 400 nucleotides or more, 450 nucleotides or more, 500 nucleotides or more, 1000 nucleotides (1 kb) or more, 5000 nucleotides (5 kb) or more, 10000 nucleotides (10 kb) or more, etc. In some instances, the 5′ and 3′ homology arms are substantially equal in length to one another. However, in some instances the 5′ and 3′ homology arms are not necessarily equal in length to one another. For example, one homology arm may be 30% shorter or less than the other homology arm, 20% shorter or less than the other homology arm, 10% shorter or less than the other homology arm, 5% shorter or less than the other homology arm, 2% shorter or less than the other homology arm, or only a few nucleotides less than the other homology arm. In other instances, the 5′ and 3′ homology arms are substantially different in length from one another, e.g. one may be 40% shorter or more, 50% shorter or more, sometimes 60% shorter or more, 70% shorter or more, 80% shorter or more, 90% shorter or more, or 95% shorter or more than the other homology arm.

The donor polynucleotide may be used in combination with an RNA-guided nuclease, which is targeted to a particular genomic sequence (i.e., genomic target sequence to be modified) by a guide RNA. A target-specific guide RNA comprises a nucleotide sequence that is complementary to a genomic target sequence, and thereby mediates binding of the nuclease-gRNA complex by hybridization at the target site. For example, the gRNA can be designed with a sequence complementary to the sequence of a minor allele to target the nuclease-gRNA complex to the site of a mutation. The mutation may comprise an insertion, a deletion, or a substitution. For example, the mutation may include a single nucleotide variation, gene fusion, translocation, inversion, duplication, frameshift, missense, nonsense, or other mutation associated with a phenotype or disease of interest. The targeted minor allele may be a common genetic variant or a rare genetic variant. In some embodiments, the gRNA is designed to selectively bind to a minor allele with single base-pair discrimination, for example, to allow binding of the nuclease-gRNA complex to a single nucleotide polymorphism (SNP). In particular, the gRNA may be designed to target disease-relevant mutations of interest for the purpose of genome editing to remove the mutation from a gene. Alternatively, the gRNA can be designed with a sequence complementary to the sequence of a major or wild-type allele to target the nuclease-gRNA complex to the allele for the purpose of genome editing to introduces a mutation into a gene in the genomic DNA of the cell, such as an insertion, deletion, or substitution. Such genetically modified cells can be used, for example, to alter phenotype, confer new properties, or produce disease models for drug screening.

In some embodiments, the RNA-guided nuclease used for genome modification is a clustered regularly interspersed short palindromic repeats (CRISPR) system Cas nuclease. Any RNA-guided Cas nuclease capable of catalyzing site-directed cleavage of DNA to allow integration of donor polynucleotides by the HDR mechanism can be used in genome editing, including CRISPR system Class 1, Type I, II, or III Cas nucleases; Class 2, Type II nuclease (such as Cas9); a Class 2, Type V nuclease (such as Cpf1), or a Class 2, Type VI nuclease (such as C2c2). Examples of Cas proteins include Cas1, Cas1B, Cas2, Cas3, Cas4, Cas5, CasSe (CasD), Cas6, Cas6e, Cas6f, Cas7, Cas8a1, Cas8a2, Cas8b, Cas8c, Cas9 (Csnl or Csxl2), CaslO, CaslOd, CasF, CasG, CasH, Csyl, Csy2, Csy3, Csel (CasA), Cse2 (CasB), Cse3 (CasE), Cse4 (CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csxl7, Csx14, CsxlO, Csxl6, CsaX, Csx3, Csxl, Csxl5, Csfl, Csf2, Csf3, Csf4, and Cul966, and homologs or modified versions thereof.

In some embodiments, a Class 1, type II CRISPR system Cas9 endonuclease is used. Cas9 nucleases from any species, or biologically active fragments, variants, analogs, or derivatives thereof that retain Cas9 endonuclease activity (i.e., catalyze site-directed cleavage of DNA to generate double-strand breaks) may be used to perform genome modification as described herein. The Cas9 need not be physically derived from an organism but may be synthetically or recombinantly produced. Cas9 sequences from a number of bacterial species are well known in the art and listed in the National Center for Biotechnology Information (NCBI) database. See, for example, NCBI entries for Cas9 from: Streptococcus pyogenes (WP 002989955, WP_038434062, WP_011528583); Campylobacter jejuni (WP_022552435, YP 002344900), Campylobacter coli (WP 060786116); Canipylobacter fetus (WP 059434633); Corynebacterium ulcerans (NC_015683, NC_017317); Corynebacterium diphtheria (NC_016782, NC_016786); Enterococcus faecalis (WP 033919308); Spiroplasma syrphidicola (NC 021284); Prevotella intermedia (NC 017861); Spiroplasma taiwanense (NC 021846); Streptococcus iniae (NC 021314); Belliella baltica (NC 018010); Psychroflexus torquisl (NC O 18721); Streptococcus thermophilus (YP 820832), Streptococcus mutans (WP 061046374, WP 024786433); Listeria innocua (NP 472073); Listeria monocytogenes (WP 061665472); Legionella pneumophila (WP 062726656); Staphylococcus aureus (WP_001573634); Francisella tularensis (WP_032729892, WP_014548420), Enterococcus faecalis (WP 033919308); Lactobacillus rhamnosus (WP 048482595, WP_032965177); and Neisseria meningitidis (WP_061704949, YP_002342100); all of which sequences (as entered by the date of filing of this application) are herein incorporated by reference in their entireties. Any of these sequences or a variant thereof comprising a sequence having at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity thereto, can be used for genome editing, as described herein. See also Fonfara et al. (2014) Nucleic Acids Res. 42(4):2577-90; Kapitonov et al. (2015) J. Bacterid. 198(5): 797-807, Shmakov et al. (2015) Mol. Cell. 60(3):385-397, and Chylinski et al. (2014) Nucleic Acids Res. 42(10):6091-6105); for sequence comparisons and a discussion of genetic diversity and phylogenetic analysis of Cas9.

The genomic target site will typically comprise a nucleotide sequence that is complementary to the gRNA and may further comprise a protospacer adjacent motif (PAM). In some embodiments, the target site comprises 20-30 base pairs in addition to a 3 or more base pair PAM. Typically, the first nucleotide of a PAM can be any nucleotide, while the two or more other nucleotides will depend on the specific Cas9 protein that is chosen. Exemplary PAM sequences are known to those of skill in the art and include, without limitation, NNG, NGN, NAG, and NGG, wherein N represents any nucleotide. In some embodiments, the allele targeted by a gRNA comprises a mutation that creates a PAM within the allele, wherein the PAM promotes binding of the Cas9-gRNA complex to the allele.

In some embodiments, the gRNA is 5-50 nucleotides, 10-30 nucleotides, 15-25 nucleotides, 18-22 nucleotides, or 19-21 nucleotides in length, or any length between the stated ranges, including, for example, 10, 11, 12, 13, 14, 15, 16, 17, 18, 19, 20, 21, 22, 23, 24, 25, 26, 27, 28, 29, 30, 31, 32, 33, 34, or 35 nucleotides in length. The guide RNA may be a single guide RNA comprising crRNA and tracrRNA sequences in a single RNA molecule, or the guide RNA may comprise two RNA molecules with crRNA and tracrRNA sequences residing in separate RNA molecules.

In another embodiment, the CRISPR nuclease from Prevotella and Francisella 1 (Cpf1, or Cas12a) is used. Cpf1 is another class 11 CRISPR/Cas system RNA-guided nuclease with similarities to Cas9 and may be used analogously. Unlike Cas9, Cpf1 does not require a tracrRNA and only depends on a crRNA in its guide RNA, which provides the advantage that shorter guide RNAs can be used with Cpf1 for targeting than Cas9. Cpf1 is capable of cleaving either DNA or RNA. The PAM sites recognized by Cpf1 have the sequences 5′-YTN-3′ (where “Y” is a pyrimidine and “N” is any nucleobase) or 5′-TTN-3′, in contrast to the G-rich PAM site recognized by Cas9. Cpf1 cleavage of DNA produces double-stranded breaks with a sticky-ends having a 4 or 5 nucleotide overhang. For a discussion of Cpf1, see, e.g., Ledford et al. (2015) Nature. 526 (7571):17-17, Zetsche et al. (2015) Cell. 163 (3):759-771, Murovec et al. (2017) Plant Biotechnol. J. 15(8):917-926, Zhang et al. (2017) Front. Plant Sci. 8:177, Fernandes et al. (2016) Postepy Biochem. 62(3):315-326; herein incorporated by reference.

C2c1 (Cas12b) is another class II CRISPR/Cas system RNA-guided nuclease that may be used. C2c1, similarly to Cas9, depends on both a crRNA and tracrRNA for guidance to target sites. See, e.g., Shmakov et al. (2015) Mol Cell. 60(3):385-397, Zhang et al. (2017) Front Plant Sci. 8:177; herein incorporated by reference.

In yet another embodiment, an engineered RNA-guided Fokl nuclease may be used. RNA-guided Fokl nucleases comprise fusions of inactive Cas9 (dCas9) and the Fokl endonuclease (FokI-dCas9), wherein the dCas9 portion confers guide RNA-dependent targeting on Fokl. For a description of engineered RNA-guided Fold nucleases, see, e.g., Havlicek et al. (2017) Mol. Ther. 25(2):342-355, Pan et al. (2016) Sci Rep. 6:35794, Tsai et al. (2014) Nat Biotechnol. 32(6):569-576; herein incorporated by reference.

In other embodiments, any other Cas enzymes and variants described in other sections of the application (all incorporated herein) can be used similarly.

In some embodiments, the RNA-guided nuclease is provided in the form of a protein, optionally where the nuclease is complexed with a gRNA to form a ribonucleoprotein (RNP) complex. In some embodiments, the RNA-guided nuclease is provided by a nucleic acid encoding the RNA-guided nuclease, such as an RNA (e.g., messenger RNA) or DNA (expression vector). In some embodiments, the RNA-guided nuclease and the gRNA are both provided by vectors, such as the vectors and the vector system described in other parts of the application (all incorporated herein by reference). Both can be expressed by a single vector or separately on different vectors. The vectors encoding the RNA-guided nuclease and gRNA may be included in the vector system comprising the engineered retron msr gene, msd gene and ret gene sequences. In some embodiments, the RNA-guided nuclease is fused to the RT and/or the msDNA.

The RNP complex may be administered to a subject or delivered into a cell by methods known in the art, such as those described in U.S. Pat. No. 11,390,884, which is incorporated by reference herein in its entirety. In some embodiments, the endonuclease/gRNA ribonucleoprotein (RNP) complexes are delivered to cells by electroporation. Direct delivery of the RNP complex to a subject or cell eliminates the need for expression from nucleic acids (e.g., transfection of plasmids encoding Cas9 and gRNA). It also eliminates unwanted integration of DNA segments derived from nucleic acid delivery (e.g., transfection of plasmids encoding Cas9 and gRNA). An endonuclease/gRNA ribonucleoprotein (RNP) complex usually is formed prior to administration.

Codon usage may be optimized to further improve production of an RNA-guided nuclease and/or reverse transcriptase (RT) in a particular cell or organism. For example, a nucleic acid encoding an RNA-guided nuclease or reverse transcriptase can be modified to substitute codons having a higher frequency of usage in a yeast cell, a bacterial cell, a human cell, a non-human cell, a mammalian cell, a rodent cell, a mouse cell, a rat cell, or any other host cell of interest, as compared to the naturally occurring polynucleotide sequence. When a nucleic acid encoding the RNA-guided nuclease or reverse transcriptase is introduced into cells, the protein can be transiently, conditionally, or constitutively expressed in the cell.

In some embodiments, the engineered retron used for genome editing with nuclease genome editing systems can further include accessory or enhancer proteins for recombination. Examples of recombination enhancers can include nonhomologous end joining (NHEJ) inhibitors (e.g., inhibitor of DNA ligase IV, a KU inhibitor (e.g., KU70 or KU80), a DNA-PKc inhibitor, or an artemis inhibitor) and homologous directed repair (HDR) promoters, or both, that can enhance or improve more precise genome editing and/or the efficiency of homologous recombination. In some embodiments, the recombination accessory or enhancers can comprise C-terminal binding protein interacting protein (CtIP), cyclinB2, Rad family members (e.g. Rad50, Rad51, Rad52, etc).

CtIP is a transcription factor containing C2H2 zinc fingers that are involved in early steps of homologous recombination. Mammalian CtIP and its orthologs in other eukaryotes promote the resection of DNA double-strand breaks and are essential for meiotic recombination. HDR may be enhanced by using Cas9 nuclease associated (e.g. fused) to an N-terminal domain of CtIP, an approach that forces CtIP to the cleavage site and increases transgene integration by HDR. In some embodiments, an N-terminal fragment of CtIP, called HE for HDR enhancer, may be sufficient for HDR stimulation and requires the CtIP multimerization domain and CDK phosphorylation sites to be active. HDR stimulation by the Cas9-HE fusion depends on the guide RNA used, and therefore the guide RNA will be designed accordingly.

Using the gene editing system described herein, any target gene or sequence in a host cell can be edited or modified for a desired trait, including but not limited to: Myostatin (e.g., GDF8) to increase muscle growth; Pc POLLED to induce hairlessness; KISS1R to induce bore taint; Dead end protein (dnd) to induce sterility; Nano2 and DDX to induce sterility; CD163 to induce PRRSV resistance; RELA to induce ASFV resilience; CD18 to induce Mannheimia (Pasteurella) haemolytica resilience; NRAMPl to induce tuberculosis resilience; Negative regulators of muscle mass (e.g., Myostatin) to increase muscle mass.

### Diseases and Disorders

Provided herein are methods of treating a disease or disorder, the methods comprising administering to a subject in need thereof a pharmaceutical composition of the present disclosure which includes a retron-based gene editing system and/or components thereof. In various embodiments of the invention, target genome or epigenetic modifications include cells with monogenic diseases or disorders. Various monogenic diseases include but are not limited to: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; Tay-Sachs Disease; hereditary tyrosinemia I; Influenza; SARS-CoV-2; Alzheimer's disease; Parkinson's disease.

Target sequences related to certain diseases and disorders are known in some cases. Target sequences or target editing sites include disease-associated or causative mutations for one or more of 10,000 monogenic disorders. A list of target sequences can be generated based on the monogenic disorders. Common genetic disorders that may be correctable by the gene editing systems described here including but are not limited to: Adenosine Deaminase (ADA) Deficiency; Alpha-1 Antitrypsin Deficiency; Cystic Fibrosis; Duchenne Muscular Dystrophy; Galactosemia; Hemochromatosis; Huntington's Disease; Maple Syrup Urine Disease; Marfan Syndrome; Neurofibromatosis Type 1; Pachyonychia Congenita; Phenylkeotnuria; Severe Combined Immunodeficiency; Sickle Cell Disease; Smith-Lemli-Opitz Syndrome; and Tay-Sachs Disease. In other embodiments, the disease-associated gene can be associated with a polygenic disorder selected from the group consisting of: heart disease; high blood pressure; Alzheimer's disease; arthritis; diabetes; cancer; and obesity.

The gene editing systems disclosed herein may also be used to treat the following genetic disorders by editing a defect in the disease-associated gene, as follows:

| Genetic disease | Disease gene |
|---|---|
| Arenoleukodystrophy (ALD) | ABCD1 |
| Agammaglobulinemia non-Bruton type | IGHM |
| Alport syndrome | COL4A5 |
| Amyloid neuropathy - Andrade disease | TTR |
| Angioneurotic oedema | C1NH |
| Alpha1-antitrypsin deficiency | SERPINEA 1 |
| Bartter syndrome type 4 | BSND |
| Blepharophimosis - ptosis - epicanthus inversus | FOXL2 |
| syndrome (BEPS) | |
| Brugada sindrome - Long QT syndrome-3 | SCN5A |
| Bruton agammaglobulinemia tyrosine kinase | BTK |
| Ceroid lipofuscinosis neuronal type 2 | CLN2 |
| Charcot Marie Tooth type 1A (CMT1A) | PMP22 |
| Charcot Marie Tooth type X (CMTX) | CMTX |
| Chronic granulomatous disease (CGD) | CYBB |
| Cystic Fibrosis (CF) | CFTR |
| Congenital adrenal hyperplasia (CAH) | CYP21A2 |
| Congenital disorder of glycosylation type Ia (CDG Ia) | PMM2 |
| Congenital fibrosis of extraocular muscles 1 (CFEOM1) | KIF21A |
| Crigler-Najjar syndrome | UGT1A1 |
| Deafness, autosomal recessive | CX26 |
| Diamond-Blackfan anemia (DBA) | RPS19 |
| Duchenne-Becker muscular dystrophy (DMD/DMB) | DMD |
| Duncan disease - X-linked lymphoproliferative syndrome | SH2D1A |
| (XLPD) | |
| Ectrodactyly ectodermal dysplasia and cleft lip/palate | p63 |
| syndrome (EEC) | |
| Epidermolysis bullosa dystrophica/pruriginosa | COL7A1 |
| Exostoses multiple type I (EXT1) | EXT1 |
| Exostoses multiple type II (EXT2) | EXT2 |
| Facioscapulohumeral muscular dystrophy | FRG1 |
| Factor VII deficiency | F7 |
| Familial Mediterranean Fever (FMF) | MEFV |
| Fanconi anemia A | FANCA |
| Fanconi anemia G | FANCG |
| Fragile-X | FRAXA |
| Gangliosidosis (GM1) | GLB1 |
| Gaucher disease (GD) | GBA |
| Glanzmann thrombasthenia | ITGA2B |
| Glucose-6-phosphate dehydrogenase deficiency | G6PD |
| Glutaric acidemia I | GCDH |
| Haemophilia A | F8 |
| Haemophilia B | F9 |
| Hand-foot-uterus syndrome | HOXD13 |
| Hemophagocytic lymphohistiocytosis familial, type 2 | PRF1 |
| (FHL2) | |
| Hypomagnesaemia primary | CLDN16 |
| HYPOPHOSPHATASIA | ALPL |
| Holt-Oram Sindrome (HOS) | TBX5 |
| Homocystinuria | MTHFR |
| Incontinentia pigmenti | NEMO |
| Lesch-Nyhan syndrome | HPRT |
| Limb-girdle muscular dystrophy type 2C (LGMD2C) | SGCG |
| Long QT syndrome-1 | KCNQ1 |
| Mannosidosis Alpha | MAN2B1 |
| Marfan syndrome | FBN1 |
| Methacrylic Aciduria, deficiency of beta- | HIBCH |
| hydroxyisobutyryl-CoA deacylase | |
| Mevalonic aciduria | MVK |
| Myotonic dystrophy (DM) | DMPK |
| Myotonic dystrophy type 2 (DM2) | ZNF9 |
| Mucopolysaccharidosis Type I - Hurler syndrome | IDUA |
| Mucopolysaccharidosis Type IIIA - Sanfilippo sindrome | SGSH |
| A (MPS3A) | |
| Mucopolysaccharidosis Type IIIB - Sanfilippo sindrome | NAGLU |
| B (MPS3B) | |
| Mucopolysaccharidosis Type VI (MPS VI) - Maroteaux- | ARSB |
| Lamy Syndrome | |
| Neuronal ceroid lipofuscinosis 1 - Batten's disease | PPT1 |
| (CLN1) | |
| Niemann-Pick disease | SMPD1 |
| Noonan sindrome | PTPN11 |
| Pancreatitis, hereditary (PCTT) | PRSS1 |
| Paramyotonia congenita (PMC) | SCN4A |
| Phenylketonuria | PAH |
| Polycystic kidney disease type 1 (PKD1) | PKD1 |
| Polycystic kidney disease type 2 (PKD2) | PKD2 |
| Polycystic kidney and hepatic disease-1 (ARPKD) | PKHD1 |
| Schwartz-Jampel/Stuve-Wiedemann syndrome | LIFR |
| Sickle cell anemia | HBB |
| Synpolydactyly (SPD1) | HOXA13 |
| Smith-Lemli-Opitz syndrome | DHCR7 |
| Spastic paraplegia type 3 | SPG3A |
| Spinal Muscular Atrophy (SMA) | SMN |
| Spinocerebellar ataxia 3 (SCA3) | ATXN3 |
| Spinocerebellar ataxia 7 (SCA7) | ATXN7 |
| Stargardt disease | ABCA4 |
| Tay Sachs (TSD) | HEXA |
| Thalassemia-α mental retardation syndrome | ATRX |
| Thalassemia-β | HBB |
| Torsion dystonia, early onset (EOTD) | DYT1 |
| Tyrosinaemia type 1 | FAH |
| Tuberosclerosis 1 | TSC1 |
| Tuberosclerosis 2 | TSC2 |
| Wiskott-Aldrich Sindrome (WAS) | WAS |

In addition, the gene editing systems disclosed herein may also be used to treat the following genetic disorders by editing a defect in the disease-associated gene, or in more than one gene associated with a particular disorders, as follows:

| | B | | | | |
|---|---|---|---|---|---|
| | Disease- | C | D | E | F |
| A | associated | Most common | Encoded product | Accession | Type of product |
| Genetic disease | genes | of (B) | of (C) | No. of (C) | of (C) |
| Adrenal hyperplasia due | CYP21A2 | CYP21A2 | Cytochrome | P08686 | Enzyme |
| to 21-hydroxylase | | (196) | P450 family 21 | | |
| deficiency (21-OHD CAH) | | | subfamily A | | |
| | | | member 2 | | |
| Aicardi-Goutières | ADAR; | RNASEH2B | Ribonuclease H2 | Q5TBB1 | Enzyme |
| syndrome encephalopathy | IFIH1; | (28) | subunit B | | |
| | RNASEH2A; | | | | |
| | RNASEH2B; | | | | |
| | RNASEH2C; | | | | |
| | SAMHD1; | | | | |
| | TREX1 | | | | |
| Alpha-1-antitrypsin | SERPINA1 | SERPINA1 | Serpin family A | P01009 | Enzyme |
| (A1AT) deficiency (AATD) | | (83) | member 1 | | inhibitor |
| Arrhythmogenic right | 13 different | PKP2 (138) | Plakophilin 2 | Q99959 | Adhesion |
| ventricular | genes linked | | | | protein in |
| cardiomyopathy/ | to this | | | | junctions and |
| dysplasia (ARVC, ARVD) | disorder, | | | | intermediate |
| | so far. | | | | filaments. |
| Autosomal dominant | BICC1; | PKD1 (1154) | Polycystin 1 | P98161 | Subunit of ion |
| polycystic kidney | GANAB; | | | | channel |
| disease (ADPKD) | PKD1; | | | | complex |
| | PKD2 | | | | |
| Brugada syndrome | 22 different | SCN5A (725) | Sodium voltage-gated | Q14524 | Ion channel |
| ventricular fibrillation | genes linked | | channel alpha subunit | | |
| | to this | | 5 | | |
| | disorder, | | | | |
| | so far | | | | |
| Catecholaminergic | CALM1; | RYR2 (288) | Ryanodine receptor 2 | Q92736 | Ion channel |
| polymorphic ventricular | CALM2; | | | | |
| tachycardia (CPVT) | CALM3; | | | | |
| | CASQ2; | | | | |
| | RYR2; | | | | |
| | TECRL; | | | | |
| | TRDN | | | | |
| Charcot- Marie-Tooth^{d} | 75 different | PMP22 (63) | Peripheral myelin | Q01453 | Ill-defined |
| disease/Hereditary | genes linked | | protein 22^{d} | | role in myelin |
| motor and sensory | to this | | | | and Schwann |
| neuropathy | disorder, | | | | cells |
| | so far. | | | | |
| Congenital adrenal | CYP11B1; | CYP21A2 | Cytochrome P450 | P08686 | Enzyme |
| hyperplasia (CAH) | CYP17A1; | (214) | family 21 subfamily A | | |
| | CYP21A2; | | member 2 | | |
| | HSD3B2; | | | | |
| | POR; STAR | | | | |
| Congenital sucrase- | SI | SI (23) | Sucrase-isomaltase | P14410 | Enzyme |
| isomaltase deficiency | | | | | |
| (CSID) | | | | | |
| Congenital bilateral | CFTR; | CFTR (120) | Cystic fibrosis | Q20BH0 | Ion channel |
| absence of vas deferens | ADGRG2 | | transmembrane | | |
| | | | conductance regulator | | |
| Cystic fibrosis | CFTR; | CFTR (1053) | Cystic fibrosis | Q20BH0 | Ion channel |
| | CLCA4; | | transmembrane | | |
| | DCTN4; | | conductance regulator | | |
| | STX1A; | | | | |
| | TGFB1 | | | | |
| Cystinuria-lysinuria | SLC3A1; | SLC7A9 (83) | Solute carrier | P82251 | Membrane |
| syndrome/Cystinuria | SLC7A9 | | family 7 member 9 | | transporter |
| Cytomegalic congenital | NR0B1 | NR0B1 (112) | Nuclear receptor | P51843 | Nuclear |
| adrenal hypoplasia | | | subfamily 0 group B | | receptor |
| (AHC) (subtype of | | | member 1 | | |
| congenital adrenal | | | | | |
| hypoplasia) | | | | | |
| Dentinogenes is | DSPP | DSPP (11) | Dentin sialophospho- | Q9NZW4 | Seeds |
| imperfecta (DGI) (all | | | protein | | biomineralization, |
| types) | | | | | Dentinogenesis |
| Duchenne muscular | DMD; | DMD (830) | Dystrophin | P11532 | Structural |
| dystrophy (DMD) | LTBP4 | | | | protein |
| Dysbetalipoproteinemia/ | APOE | APOE (42) | Apolipoprotein E | P02649 | Lipid carrier, |
| Hyperliproteinemia | | | | | lipoprotein |
| type 3 | | | | | |
| Ehlers-Danlos syndrome | COL1A1; | COL5A1 | Collagen type V alpha | P20908, | Structural |
| | COL5A1; | (106) | 1 chain, collagen type | P05997 | protein |
| | COL5A2 | | V alpha 2 chain | | |
| Familial adenomatous | APC; | APC (539) | Adenomatous | P25054 | Tumor |
| polyposis (FAP) | MUTYH | | polyposis coli protein | | suppressor, |
| | | | | | regulatory |
| | | | | | protein |
| Gardner syndrome | APC | APC (539) | Adenomatous | P25054 | Tumor |
| (subtype of familial | | | polyposis coli protein | | suppressor, |
| adenomatous polyposis) | | | | | associated with |
| | | | | | microtublules |
| Familial cerebral | CCM2; | KRIT1 (80) | Krev interaction | O00522 | Regulatory |
| cavernous malformation | KRIT1; | | trapped protein 1 | | protein |
| | PDCD10 | | | | |
| Familial hypocalciuric | CASR | CASR (373) | Calcium sensing | P41180 | G protein- |
| hypercalcemia type 1 | | | receptor | | coupled |
| (FHH) | | | | | receptor |
| Famililal | APOB; | LDLR (1254) | Low density | P01130 | Lipoprotein |
| hypercholesterolemia | LDLR; | | lipoprotein receptor | | receptor |
| | LDLRAP1; | | | | |
| | PCSK | | | | |
| Familial isolated | 45 different | TTN (672) | Titin | Q8WZ42 | Muscle |
| dilated cardiomyopathy | genes linked | | | | protein |
| | to this | | | | |
| | disorder, | | | | |
| | so far. | | | | |
| Familial long QT | 19 different | KCNQ1 (448) | Potassium voltage- | P51787 | Ion Channel |
| syndrome (LQTS), | genes linked | | gated channel | | |
| including Romano-Ward | to this | | subfamily Q member 1 | | |
| syndrome | disorder, | | | | |
| | so far. | | | | |
| Fragile X syndrome/ | FMR1 | FMR1 (7) | Fragile X mental | Q06787 | Regulator of |
| Martin-bell syndrome | | | retardation 1 | | mRNA biology |
| Glucose-6-phosphate | G6PD | G6PD (218) | Glucose-6-phosphate | P11413 | Enzymes |
| dehydrogenase | | | 1 dehydrogenase | | |
| deficiency | | | | | |
| Glycogen storage | 27 different | AGL (117) | Glycogen debranching | P35573 | Enzyme |
| disease | genes linked | | enzyme | | |
| | to this | | | | |
| | disorder, | | | | |
| | so far. | | | | |
| GM2 gangliosidosis | GM2A; | HEXA (124) | Hexosaminidase | P06865 | Enzyme |
| | HEXA; | | subunit alpha | | |
| | HEXB | | | | |
| Hemochromatosis | BMP6; | HFE (43) | Hereditary | Q30201 | Binds |
| | HAMP; | | hemochromatosis | | transferrin |
| | HFE; HJV; | | protein | | receptor |
| | SLC40A1; | | | | |
| | TFR2 | | | | |
| Hemolytic anemia due | PKLR | PKLR (237) | Pyruvate kinase | P30613 | Enzyme |
| to red cell pyruvate | | | | | |
| kinase deficiency | | | | | |
| Hemophilia A and B | F8; F9 | F8 (1898) | Coagulation factor | P00451 | Cofactor for |
| | | | VIII | | factor IXa |
| Hemophilia A | F8 | F8 (3364) | Coagulation factor | P00451 | Cofactor for |
| | | | VIII | | factor IXa |
| Hemorrhagic | ACVRL1; | ENG (187) | Endoglin | P17813 | Regulation of |
| telangiectasia/Osler | ENG; | | | | angiogenesis |
| Weder Rendu disease | GDF2; | | | | |
| | SMAD4 | | | | |
| Hereditary angioedema | ANGPT1; | SERPING1 | Serpin family G | P05155 | Enzyme |
| (HAE)/Angioneurotic | F12; PLG; | (252) | member 1 | | inhibitor |
| edema | SERPING1 | | | | |
| Hereditary breast | 14 different | BRCA1 | Breast cancer type 1 | P38398 | E3 ubiquitin- |
| and ovarian cancer | genes linked | (1262) | susceptibility protein | | protein ligase |
| syndrome | to this | | | | |
| | disorder | | | | |
| | so far. | | | | |
| Hereditary fructose | ALDOB | ALDOB (32) | Aldolase, fructose- | P05062 | Enzymes |
| intolerance/Fructosemia | | | bisphosphate B | | |
| Hereditary xanthinuria/ | MOCOS; | MOCOS (8); | Molybdenum cofactor | Q9C5X8, | Enzymes |
| Xanthine stone disease | XDH | XDH (17) | sulfurase, xanthine | P47989 | |
| | | | dehydrogenase | | |
| Hypohidrotic ectodermal | 10 different | EDA (199) | Ectodysplasin A | Q92838 | Cytokine |
| dysplasia (HED) | genes linked | | | | |
| | to this | | | | |
| | disorder, | | | | |
| | so far. | | | | |
| Iminoglycinuria | SLC36A2; | SLC36A2 (1) | Solute carrier family | Q495M3 | Membrane |
| | SLC6A18; | | 36 member 2 | | transporter |
| | SLC6A19; | | | | |
| | SLC6A20 | | | | |
| Li-Fraumeni syndrome | CDKN2A; | TP53 (417) | Tumor protein p53 | P04637 | Tumor |
| sarcoma, breast, | CHEK2; | | | | suppressor, |
| leukemia, and adrenal | MDM2; | | | | gene |
| gland (SBLA) syndrome | TP53 | | | | regulation |
| Long chain 3- | HADHA | HADHA (35) | Hydroxyacyl-CoA | P40939 | Enzyme |
| hydroxyacyl-CoA | | | dehydrogenase | | |
| dehydrogenase | | | trifunctional | | |
| deficiency (LCHAD) | | | multienzyme complex | | |
| | | | subunit alpha | | |
| Lynch syndrome | 11 different | MSH2 (34) | DNA mismatch repair | P43246 | DNA repair, |
| | genes linked | | protein Msh2 | | binds DNA, |
| | to this | | | | ATPase |
| | disorder | | | | |
| | so far | | | | |
| Marfan syndrome A | FBN1; | FBN1 (1893) | Fibrillin 1 | P35555 | Structural |
| | TGFBR2 | | | | protein, |
| | | | | | extracellular |
| | | | | | matrix |
| Maternal | PAH | PAH (690) | Phenylalanine | P00439 | Enzyme |
| phenylketonuria/ | | | hydroxylase | | |
| Phenylketonuric | | | | | |
| embryopathy | | | | | |
| Medium chain acyl-CoA | ACADM | ACADM | Acyl-CoA | P11310 | Enzyme |
| dehydrogenase | | (136) | dehydrogenase | | |
| deficiency (MCADD) | | | medium chain | | |
| Mucolipidosis type III | GNPTAB | GNPTAB (68) | N-acetylglucosamine | Q3T906 | Enzyme |
| (ML3) alpha/beta | | | 1 phosphate | | |
| | | | transferase, Subunits | | |
| | | | alpha and beta | | |
| Mucopolysaccharidosis | GALNS | GALNS (269) | Galactosamine (N- | P34059 | Enzyme |
| type 4A (MPS4A)/Morquio | | | acetyl)-6-sulfatase | | |
| disease type A | | | | | |
| Multiple endocrine | RET | RET (130) | Ret proto-oncogene | P07949 | Receptor |
| neoplasia type 2 | | | receptor tyrosine | | tyrosine |
| | | | kinase | | kinase |
| Multiple epiphyseal | COL2A1; | COMP (155) | Cartilage oligomeric | P49747 | Structural |
| dysplasia (MED) | COL9A1; | | matrix protein | | protein |
| | COL9A2; | | | | |
| | COL9A3/ | | | | |
| | collagen | | | | |
| | type IX | | | | |
| | alpha 3 | | | | |
| | chain; | | | | |
| | COMP; | | | | |
| | KIF7; | | | | |
| | MATN3; | | | | |
| | SLC26A2 | | | | |
| Neurofibromatosis | NF1 | NF1 (1208) | Neurofibromin 1 | P21359 | Regulator of |
| type 1 (NF1)/Von | | | | | Ras GTPase |
| Recklinghausen | | | | | activity |
| disease | | | | | |
| Oculocutaneous | LRMDA; | TYR (352) | Tyrosinase | P14679 | Enzyme |
| albinism (OCA) | MC1R; | | | | |
| | OCA2; | | | | |
| | SLC24A5; | | | | |
| | SLC45A2; | | | | |
| | TYR; | | | | |
| | TYRP1 | | | | |
| Osteogenesis imperfecta/ | 15 different | COL1A1 | Collagen type I alpha | P02452, | Structural |
| brittle bone disease | genes linked | (547); | 1 chain, collagen type | P08123 | protein |
| | to this | COL1A2 | I alpha 2 chain | | |
| | disorder, | (466) | | | |
| | so far. | | | | |
| Pendred syndrome (PDS)/ | FOXI1; | SLC26A4 | Solute carrier family | O43511 | Membrane |
| Deafness with goiter | KCNJ10; | (404) | 26 member 4 | | transporter |
| | SLC26A4 | | | | |
| Phenylketonuria (PKU)/ | PAH | PAH (690) | Phenylalanine | P00439 | Enzyme |
| Phenylalanine | | | hydroxylase | | |
| hydroxylase deficiency | | | | | |
| (PAH deficiency) | | | | | |
| Proximal spinal | NAIP; | SMN1 (47) | Survival motor neuron | Q16637 | RNA splicing |
| muscular atrophy (SMA) | SMN1; | | protein | | |
| | SMN2 | | | | |
| Retinitis Pigmentosa | 82 different | RHO (204) | Rhodopsin | P08100 | G-protein |
| (RP) | genes linked | | | | coupled |
| | to this | | | | receptor |
| | disorder, | | | | |
| | so far. | | | | |
| Recessive X-linked | STS | STS (28) | Steroid sulfatase | P08842 | Enzyme |
| ichthyosis (XLI) | | | | | |
| Retinoblastoma | NMYC; | RB1 (292) | RB transcriptional | P06400 | Tumor |
| (RB bilateral (40% | RB1 | | corepressor 1 | | suppressor, |
| of cases) and | | | | | cell cycle |
| unilateral (60% of | | | | | regulation |
| cases-de novo mutation) | | | | | |
| Rett syndrome | MECP2 | MECP2 (246) | Methyl-CpG binding | P51608 | Binds to |
| | | | protein 2 | | methylated |
| | | | | | DNA, gene |
| | | | | | regulation |
| Sickle cell anemia | HBB | HBB (433) | Hemoglobin subunit | P68871 | Oxygen |
| | | | beta | | carrier |
| Sotos syndrome/cerebral | APC2; | NSD1 (228) | Nuclear receptor | Q96L73 | Enzyme |
| gigantism | NSD1; | | binding SET domain | | |
| | SETD2 | | protein 1 | | |
| Stargardt disease/ | ABCA4; | ABCA4 (789) | ATP binding cassette | P78363 | Membrane |
| Fundus flavimaculatus | CNGB3; | | subfamily A member 4 | | transporter |
| | ELOVL4; | | | | |
| | PROM1; | | | | |
| | PRPH2 | | | | |
| Stickler syndrome/ | COL11A1; | COL2A1 | Collagen type II alpha | P02458 | Structural |
| hereditary progressive | COL2A1; | (335) | 1 chain | | protein |
| arthroophthalmopathy | COL11A2; | | | | |
| | COL9A1; | | | | |
| | COL9A2; | | | | |
| | COL9A3; | | | | |
| | LOXL3 | | | | |
| Supravalvular aortic | ELN | ELN (25) | Elastin | P15502 | Structural |
| stenosis (SVAS) | | | | | protien |
| β-Thalassemia | HBB | HBB (434) | Hemoglobin B chain | P68871 | Oxygen |
| | | | | | carrier |
| Tibial muscular | TTN | TTN (53) | Titin | Q8WZ42 | Muscle |
| dystrophy/Upp myopathy | | | | | protein |
| Tuberous sclerosis | TSC1; | TSC2 (518) | Tuberin | P49815 | Tumor |
| complex/Bourneville | TSC2 | | | | suppressor, |
| syndrome | | | | | Regulation of |
| | | | | | mTORC1 |
| | | | | | signaling |
| Von-Hippel Lindau | VHL | VHL (218) | Von Hippel-Lindau | P40337 | Tumor |
| disease | | | tumor suppressor | | suppressor, |
| | | | | | role in E3 |
| | | | | | ubiquitin |
| | | | | | ligase |
| | | | | | complex |
| Von Willebrand | VWF | VWF (636) | Von Willebrand factor | P04275 | Collagen |
| disease | | | | | binding, |
| | | | | | chaperone for |
| | | | | | coagulation |
| | | | | | factor VIII |
| X-linked | ABCD1 | ABCD1 (425) | ATP binding cassette | P33897 | Membrane |
| adrenoleukodystrophy | | | subfamily D member 1 | | transporter |
| (ALD) | | | | | |

Accordingly, to treat one or more such diseases or disorders, in various aspects of the invention, one or more targeted polynucleotide sequence related to certain diseases and disorders, e.g., a genetic mutation, is contacted by a retron-based gene editing system disclosed herein; and a guide RNA, wherein the guide RNA comprises a complementary sequence to that of a targeted polynucleotide sequence.

In some embodiments, the guide RNA directs a programmable nuclease of a retron-based editing system to the target site or the targeted polynucleotide sequence; and optionally forms a ribonucleoprotein complex with the polypeptide and the guide RNA.

Additional therapeutic applications for the genome editing systems disclosed herein include base editing, prime editing, gene insertions and/or deletions.

Diagnostic applications for the genome editing system include probes, diagnostics, theranostics.

The editing system comprising the heterologous nucleic acid sequence can be used in a variety of applications, several non-limiting examples of which are described herein. In general, the editing system can be used in any suitable organism. In some embodiments, the organism is a eukaryote.

In some embodiments, the organism is an animal. In some embodiments, the animal is a fish, an amphibian, a reptile, a mammal, or a bird. In some embodiments, the animal is a farm animal or agriculture animal. Non-limiting examples of farm and agriculture animals include horses, goats, sheep, swine, cattle, llamas, alpacas, and birds, e.g., chickens, turkeys, ducks, and geese. In some embodiments, the animal is a non-human primate, e.g., baboons, capuchin monkeys, chimpanzees, lemurs, macaques, marmosets, tamarins, spider monkeys, squirrel monkeys, and vervet monkeys. In some embodiments, the animal is a pet. Non-limiting examples of pets include dogs, cats, horses, rabbits, ferrets, gerbils, hamsters, chinchillas, fancy rats, guinea pigs, canaries, parakeets, and parrots.

In some embodiments, the organism is a plant. Plants that may be transfected with an Cas12a editing system include monocots and dicots. Particular examples include, but are not limited to, corn (maize), sorghum, wheat, sunflower, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers. Vegetables include, but are not limited to, crucifers, peppers, tomatoes, lettuce, green beans, lima beans, peas, and members of the genus Cucumis such as cucumber, cantaloupe, and musk melon. Ornamentals include, but are not limited to, azalea, hydrangea, hibiscus, roses, tulips, daffodils, petunias, carnation, poinsettia, and chrysanthemum.

In some embodiments, heterologous nucleic acid sequences can be added to the subject editing system to provide a cell with a heterologous nucleic acid encoding a protein or regulatory RNA of interest, a cellular barcode, a donor polynucleotide suitable for use in gene editing, e.g., by homology directed repair (HDR) or recombination-mediated genetic engineering (recombineering), or a CRISPR protospacer DNA sequence for use in molecular recording, as discussed further below. In embodiments relating to retron-based gene editing systems, such heterologous sequences may be inserted, for example, into the msr locus or the msd locus such that the heterologous sequence is transcribed by the retron reverse transcriptase as part of the msDNA product.

In some embodiments, the editing systems described herein may be used for research tools, such as kits, functional genomics assays, and generating engineered cell lines and animal models for research and drug screening. The kit may comprise one or more reagents in addition to the editing system, such as a buffer, a control reagent, a control vector, a control RNA polynucleotide, a reagent for in vitro production of the polypeptide from DNA, and adaptors for sequencing. A buffer can be, for example, a stabilization buffer, a reconstituting buffer, a diluting buffer, a wash buffer, or a buffer for introducing a polypeptide and/or polynucleotide of the kit into a cell. In some instances, a kit can comprise one or more additional reagents specific for plants. One or more additional reagents for plants can include, for example, soil, nutrients, plants, seeds, spores, Agrobacterium, a T-DNA vector, and a pBINAR vector.

### Production of Protein or RNA

In some embodiments, the single-stranded msDNA generated by the engineered retron of the invention can be used to produce a desired product of interest in cells.

In some embodiments, the retron is engineered with a heterologous sequence encoding a polypeptide of interest to allow production of the polypeptide from the retron msDNA generated in a cell. The polypeptide of interest may be any type of protein/peptide including, without limitation, an enzyme, an extracellular matrix protein, a receptor, transporter, ion channel, or other membrane protein, a hormone, a neuropeptide, an antibody, or a cytoskeletal protein, a functional fragment thereof, or a biologically active domain of interest. In some embodiments, the protein is a therapeutic protein, therapeutic antibody for use in treatment of a disease, or a template to fix a mutation or mutated exon in the genome.

Non-limiting examples of polypeptides of interest include: growth hormones, insulin-like growth factors (IGF-1), Fat-1, Phytase, xylanase, beta-glucanase, Lysozyme or lysostaphin, Histone deacetylase such as HDAC6, CD163, etc.

In other embodiments, the retron is engineered with a heterologous sequence encoding an RNA of interest to allow production of the RNA from the retron in a cell. The RNA of interest may be any type of RNA including, without limitation, a RNA interference (RNAi) nucleic acid or regulatory RNA such as, but not limited to, a microRNA (miRNA), a small interfering RNA (siRNA), a short hairpin RNA (shRNA), a small nuclear RNA (snRNA), a long non-coding RNA (lncRNA), an antisense nucleic acid, and the like.

### Recombineering

Recombineering (recombination-mediated genetic engineering) can be used in modifying chromosomal as well as episomal replicons in cells, for example, to create gene replacements, gene knockouts, deletions, insertions, inversions, or point mutations. Recombineering can also be used to modify a plasmid or bacterial artificial chromosome (BAC), for example, to clone a gene or insert markers or tags.

The engineered retrons described herein can be used in recombineering applications to provide linear single-stranded or double-stranded DNA for recombination. Homologous recombination may be mediated by bacteriophage proteins such as RecE/RecT from Rac prophage or Redobd from bacteriophage lambda. The linear DNA should have sufficient homology at the 5′ and 3′ ends to a target DNA molecule present in a cell (e.g., plasmid, BAC, or chromosome) to allow recombination.

The linear double-stranded or single-stranded DNA molecule used in recombineering (i.e. donor polynucleotide) comprises a sequence having the intended edit to be inserted flanked by two homology arms that target the linear DNA molecule to a target site for homologous recombination. Homology anns for recombineering typically range in length from 13-300 nucleotides, or 20 to 200 nucleotides, including any length within this range such as 13, 14, 15, 16, 17, 18, 19, 20, 22, 24, 26, 28, 30, 32, 34, 36, 38, 40, 42, 44, 46, 48, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 nucleotides in length. In some embodiments, a homology arm is at least 15, at least 20, at least 30, at least 40, or at least 50 or more nucleotides in length. Homology arms ranging from 40-50 nucleotides in length generally have sufficient targeting efficiency for recombination; however, longer homology arms ranging from 150 to 200 bases or more may further improve targeting efficiency. In some embodiments, the 5′ homology arm and the 3′ homology arm differ in length. For example, the linear DNA may have about 50 bases at the 5′ end and about 20 bases at the 3′ end with homology to the region to be targeted.

The bacteriophage homologous recombination proteins can be provided to a cell as proteins or by one or more vectors encoding the recombination proteins, such as the vector or vector system. In some embodiments, one or more vectors encoding the bacteriophage recombination proteins are included in the vector system comprising the engineered retron msr gene, msd gene, and/or ret gene sequences. Additionally, a number of bacterial strains containing prophage recombination systems are available for recombineering, including, without limitation, DY380, containing a defective 1 prophage with recombination proteins exo, bet, and gam; EL250, derived from DY380, which in addition to the recombination genes found in DY380, also contains a tightly controlled arabinose-inducible flpe gene (flpe mediates recombination between two identical frt sites); EL350, also derived from DY380, which in addition to the recombination genes found in DY380, also contains a tightly controlled arabinose-inducible ere gene (ere mediates recombination between two identical loxP sites; SW102, derived from DY380, which is designed for BAC recombineering using a galK positive/negative selection; SW105, derived from EL250, which can also be used for galK positive/negative selection, but like EL250, contain an ara-inducible Flpe gene; and SW106, derived from EL350, which can be used for galK positive/negative selection, but like EL350, contains an ara-inducible Cre gene. Recombineering can be carried out by transfecting bacterial cells of such strains with an engineered retron comprising a heterologous sequence encoding a linear DNA suitable for recombineering. For a discussion of recombineering systems and protocols, see, e.g., Sharan et al. (2009) Nat Protoc. 4(2): 206-223, Zhang et al. (1998) Nature Genetics 20: 123-128, Muyrers et al. (1999) Nucleic Acids Res. 27: 1555-1557, Yu et al. (2000) Proc. Natl. Acad. Sci U.S.A. 97 (11):5978-5983; herein incorporated by reference.

### Molecular Recording

In some embodiments, the heterologous sequence in the engineered retron construct comprises a synthetic CRISPR protospacer DNA sequence to allow molecular recording. The endogenous CRISPR Cas1-Cas2 system is normally utilized by bacteria and archaea to keep track of foreign DNA sequences originating from viral infections by storing short sequences (i.e., protospacers) that confer sequence-specific resistance to invading viral nucleic acids within genome-based arrays. These arrays not only preserve the spacer sequences but also record the order in which the sequences are acquired, generating a temporal record of acquisition events.

This system can be adapted to record arbitrary DNA sequences into a genomic CRISPR array in the form of “synthetic protospacers” that are introduced into cells using engineered retrons. Engineered retrons carrying the protospacer sequences can be used for integration of synthetic CRISPR protospacer sequences at a specific genomic locus by utilizing the CRISPR system Cas1-Cas2 complex. Molecular recording can be used to keep track of certain biological events by producing a stable genetic memory tracking code. See, e.g., Shipman etaL. (2016) Science 353(6298): aafl 175 and International Patent Application Publication No. WO/2018/191525; herein incorporated by reference in their entireties.

In some embodiments, the CRISPR-Cas system is harnessed to record specific and arbitrary DNA sequences into a bacterial genome. The DNA sequences can be produced by an engineered retron within the cell. For example, the engineered retron can be used to produce the protospacers within the cell, which are inserted into a CRISPR array within the cell. The cell may be modified to include one or more engineered returns (or vector systems encoding them) that can produce one or more synthetic protospacers in the cell, wherein the synthetic protospacers are added to the CRISPR array. A record of defined sequences, recorded over many days, and in multiple modalities can be generated.

In some embodiments, the engineered retron comprises an msd protospacer nucleic acid region or an msr protospacer nucleic acid region. In the case of a msr protospacer nucleic acid region, the protospacer sequence is first incorporated into the msr RNA, which is reverse transcribed into protospacer DNA. Double stranded protospacer DNA is produced when two complementary protospacer DNA sequences having complementary sequences hybridize, or when a double-stranded structure (such as a hairpin) is formed in a single stranded protospacer DNA (e.g., a single msDNA can form an appropriate hairpin structure to provide the double stranded DNA protospacer).

In some embodiments, a single stranded DNA produced in vivo from a first engineered retron may be hybridized with a complementary single-stranded DNA produced in vivo from the same retron or a second engineered retron or may form a hairpin structure and then used as a protospacer sequence to be inserted into a CRISPR array as a spacer sequence. The engineered retron(s) should provide sufficient levels of the protospacer sequence within a cell for incorporation into the CRISPR array. The use of protospacers generated within the cell extends the in vivo molecular recording system from only capturing information known to a user, to capturing biological or environmental information that may be previously unknown to a user. For example, an msDNA protospacer sequence in an engineered retron construct may be driven by a promoter that is downstream of a sensor pathway for a biological phenomenon or environmental toxin. The capture and storage of the protospacer sequence in the CRISPR array records the event. If multiple msDNA protospacers are driven by different promoters, the activity of those promoters is recorded (along with anything that may be upstream of the promoters) as well as the relative order of promoter activity (based on the relative position of spacer sequences in the CRISPR array). At any point after the recording has taken place, the CRISPR array may be sequenced to determine whether a given biological or environmental event has taken place and the order of multiple events, given by the presence and relative position of msDNA-derived spacers in the CRISPR array.

In some embodiments, the synthetic protospacer further comprises an AAG PAM sequence at its 5′ end. Protospacers including the 5′ AAG PAM are acquired by the CRISPR array with greater efficiency than those that do not include a PAM sequence.

In some embodiments, Cas1 and Cas2 are provided by a vector that expresses the Cas1 and Cas2 at a level sufficient to allow the synthetic protospacer sequences produced by engineered retrons to be acquired by a CRISPR array in a cell. Such a vector system can be used to allow molecular recording in a cell that lacks endogenous Cas proteins.

### Therapeutic Applications

Also provided herein are methods of diagnosing, prognosing, treating, and/or preventing a disease, state, or condition in or of a subject, using the engineered retron of the invention.

Generally, the methods of diagnosing, prognosing, treating, and/or preventing a disease, state, or condition in or of a subject can include modifying a polynucleotide in a subject or cell thereof using a composition, system, or component thereof of the engineered retron as described herein, and/or include detecting a diseased or healthy polynucleotide in a subject or cell thereof using a composition, system, or component thereof of the engineered retron as described herein.

In some embodiments, the method of treatment or prevention can include using a composition, system, or component of the engineered retron to modify a polynucleotide of an infectious organism (e.g. bacterial or virus) within a subject or cell thereof.

In some embodiments, the method of treatment or prevention can include using a composition, system, or component of the engineered retron to modify a polynucleotide of an infectious organism or symbiotic organism within a subject.

In some embodiments, the composition, system, and components of the engineered retron can be used to develop models of diseases, states, or conditions.

In some embodiments, the composition, system, and components of the engineered retron can be used to detect a disease state or correction thereof, such as by a method of treatment or prevention described herein.

In some embodiments, the composition, system, and components of the engineered retron can be used to screen and select cells that can be used, for example, as treatments or preventions described herein.

In some embodiments, the composition, system, and components thereof can be used to develop biologically active agents that can be used to modify one or more biologic functions or activities in a subject or a cell thereof.

In general, the method can include delivering a composition, system, and/or component of the engineered retron to a subject or cell thereof, or to an infectious or symbiotic organism by a suitable delivery technique and/or composition. Once administered, the components can operate as described elsewhere herein to elicit a nucleic acid modification event. In some embodiments, the nucleic acid modification event can occur at the genomic, epigenomic, and/or transcriptomic level. DNA and/or RNA cleavage, gene activation, and/or gene deactivation can occur.

The composition, system, and components of the engineered retron as described elsewhere herein can be used to treat and/or prevent a disease, such as a genetic and/or epigenetic disease, in a subject; to treat and/or prevent genetic infectious diseases in a subject, such as bacterial infections, viral infections, fungal infections, parasite infections, and combinations thereof, to modify the composition or profile of a microbiome in a subject, which can in turn modify the health status of the subject; to modify cells ex vivo, which can then be administered to the subject whereby the modified cells can treat or prevent a disease or symptom thereof; or to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein of the engineered retron, and administering them to the subject.

Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component of the engineered retron, and comprising multiple Cas effectors.

Also provided is a method of treating a subject, e.g., a subject in need thereof, comprising inducing gene editing by transforming the subject with the Cas effector(s), and encoding and expressing in vivo the remaining portions of the composition, system, (e.g., RNA, guides), complex or component of the engineered retron. A suitable repair template may also be provided by the engineered retron as described herein elsewhere.

Also provided is a method of treating a subject, e.g., a subject in need thereof, comprising inducing transcriptional activation or repression by transforming the subject with the systems or compositions herein.

Also provided is a method of inducing one or more polynucleotide modifications in a eukaryotic or prokaryotic cell or component thereof (e.g. a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In some embodiments, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of the particle delivery system or the delivery system or the virus vector (in viral particle) of any one of the above embodiment or the cell of any one of the above embodiment in ex vivo or in vivo gene or genome editing; or for use in in vitro, ex vivo or in vivo gene therapy.

Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In some embodiments, target polynucleotide modification using the subject engineered retron and the associated composition, vectors, system and methods comprises addition, deletion, or substitution of 1-about 10k nucleotides at each target sequence of said polynucleotide of said cell(s). The modification can include the addition, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, 75, 100, 200, 250, 300, 500, 1000, 1500, 2000, 2500, 3000, 3500, 4000, 5000, 6000, 7000, 8000, 9000, 10,000 or more nucleotides at each target sequence.

In some embodiments, formation of system or complex results in cleavage, nicking, and/or another modification of one or both strands in or near (e.g. within 1, 2, 3, 4, 5, 6, 7, 8, 9, 10, 20, 50, or more base pairs from) the target sequence.

In some embodiments, a method of modifying a target polynucleotide in a cell to treat or prevent a disease can include allowing a composition, system, or component of the subject engineered retron to bind to the target polynucleotide, e.g., to effect cleavage, nicking, or other modification as the composition, system, is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a tracr mate sequence, which in turn can hybridize to a tracr sequence. In some embodiments, modification can include cleaving or nicking one or two strands at the location of the target sequence by one or more components of the composition, system, or component thereof.

In some embodiments, the engineered retron and the associated compositions, systems, vectors, uses, and methods of use, can be used to treat diseases of the circulatory system. In some embodiments, the treatment can be carried out by using an AAV or a lentiviral vector to deliver the engineered retron, composition, system, and/or vector described herein to modify hematopoietic stem cells (HSCs) or iPSCs in vivo or ex vivo. In some embodiments, the treatment can be carried out by correcting HSCs or iPSCs as to the disease using a composition, system, herein or a component thereof, wherein the composition, system, optionally includes a suitable HDR repair template (e.g., a template in the msDNA of the engineered retron).

In some embodiments, the treatment or prevention for treating a circulatory system or blood disease can include modifying a human cord blood cell. In some embodiments, the treatment or prevention for treating a circulatory system or blood disease can include modifying a granulocyte colony-stimulating factor-mobilized peripheral blood cell (mPB) with any modification described herein. In some embodiments, the human cord blood cell or mPB can be CD34+. In some embodiments, the cord blood cells or mPB cells modified are autologous. In some embodiments, the cord blood cells or mPB cells are allogenic. In addition to the modification of the disease genes, allogenic cells can be further modified using the composition, system, described herein to reduce the immunogenicity of the cells when delivered to the recipient. The modified cord blood cells or mPB cells can be optionally expanded in vitro. The modified cord blood cell(s) or mPB cells can be derived to a subject in need thereof using any suitable delivery technique.

The composition and system may be engineered to target genetic locus or loci in HSCs. In some embodiments, the components of the systems can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles, such as the lipid nanoparticle delivery system described herein. The particles may be formed by the components of the systems herein being admixed.

In some embodiments, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20):4082-9. doi: 10.1182/blood-2012-09-455204. Epub 2013 Mar. 21.

In some embodiments, the HSCs or iPSCs modified are autologous. In some embodiments, the HSCs or iPSCs are allogenic. In addition to the modification of the disease genes, allogenic cells can be further modified using the composition, system, described herein to reduce the immunogenicity of the cells when delivered to the recipient.

In some embodiments, the engineered retron and the associated compositions, systems, vectors, uses, and methods of use, can be used to treat neurological diseases. In some embodiments, the neurological diseases comprise diseases of the brain and CNS.

Delivery options for the diseases in the brain include encapsulation of the systems in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors or vector systems of the invention. In other embodiments, an artificial virus can be generated for CNS and/or brain delivery.

In some embodiments, the engineered retron and the associated compositions, systems, vectors, uses, and methods of use, can be used to treat hearing diseases or hearing loss in one or both ears. Deafness is often caused by lost or damaged hair cells that cannot relay signals to auditory neurons. In some embodiments, the composition, system, or modified cells can be delivered to one or both ears for treating or preventing hearing disease or loss by any suitable method or technique known in the art, such as US20120328580 (e.g., auricular administration), by intratympanic injection (e.g., into the middle ear), and/or injections into the outer, middle, and/or inner ear; administration in situ, via a catheter or pump (U.S. 2006/0030837) and Jacobsen (U.S. Pat. No. 7,206,639). Also see US20120328580. Cells resulting from such methods can then be transplanted or implanted into a patient in need of such treatment.

In some embodiments, the engineered retron and the associated compositions, systems, vectors, uses, and methods of use, can be used to treat diseases in non-dividing cells. Exemplary non-dividing cells include muscle cells or neurons. In such cells, homologous recombination (HR) is generally suppressed in the G1 cell-cycle phase, but can be turned back on using art-recognized methods, such as Orthwein et al. (Nature. 2015 Dec. 17; 528(7582): 422-426).

In some embodiments, the engineered retron and the associated compositions, systems, vectors, uses, and methods of use, can be used to treat diseases of the eye.

In some embodiments, the engineered retron and the associated compositions, systems, vectors, uses, and methods of use, can be used to treat muscle diseases and cardiovascular diseases.

In some embodiments, the engineered retron and the associated compositions, systems, vectors, uses, and methods of use, can be used to treat diseases of the liver and kidney.

In some embodiments, the engineered retron and the associated compositions, systems, vectors, uses, and methods of use, can be used to treat epithelial and lung diseases.

In some embodiments, the engineered retron and the associated compositions, systems, vectors, uses, and methods of use, can be used to treat diseases of the skin.

In some embodiments, the engineered retron and the associated compositions, systems, vectors, uses, and methods of use, can be used to treat cancer.

In some embodiments, the engineered retron and the associated compositions, systems, vectors, uses, and methods of use, can be used in adoptive cell therapy.

In some embodiments, the engineered retron and the associated compositions, systems, vectors, uses, and methods of use, can be used to treat infectious diseases.

In some embodiments, the engineered retron and the associated compositions, systems, vectors, uses, and methods of use, can be used to treat mitochondrial diseases.

All publications, published patent documents, and patent applications cited herein are hereby incorporated by reference to the same extent as though each individual publication, published patent document, or patent application was specifically and individually indicated as being incorporated by reference.

### O. Sequences

### Sequence Summary Table

The following sequences form part of the specification.

| SEQ ID NO: | Description |
|---|---|
| 1-7257 | Table A Sequences (AA) - Retron reverse transcriptase |
| | amino acid sequences computationally discovered pursuant |
| | to Example 3 and tested/evaluated throughout the Examples |
| | (sequences included in Sequence Listing filed with the |
| | Specification identified as |
| | RTX009P1_Sequence_Listing_26Jul23.xml) |
| 7258-14498 | Table A Sequences (NT) - Retron reverse transcriptase |
| | nucleotide sequences computationally discovered pursuant |
| | to Example 3 and tested/evaluated throughout the Examples |
| | (sequences included in Sequence Listing filed with the |
| | Specification identified as |
| | RTX009P1_Sequence_Listing_26Jul23.xml) |
| 14499-19108 | Table B Sequences (NT) - Retron non-coding RNA |
| | (ncRNA) nucleotide sequences discovered pursuant to |
| | Example 3 and tested/evaluated throughout the Examples |
| | (sequences included in Sequence Listing filed with the |
| | Specification identified as |
| | RTX009P1_Sequence_Listing_26Jul23.xml) |
| 19109-19125 | Examples: Select RT and ncRNA sequences (sequence |
| | included in this Specification) |
| 19126-19211 | Examples: RNA sequences (sequence included in this |
| | Specification) |
| 19212-19217 | Examples: Primer sequences (sequence included in this |
| | Specification) |
| 19218-19221 | Examples: qPCR primer sequences (sequence included in |
| | this Specification) |
| 19222-12261 | Examples: RT variants (coding sequences) (sequence |
| | included in this Specification) |
| 12262-19364 | Examples: Plasmid sequences (sequence included in this |
| | Specification) |
| 19365-19398 | Table C - Retron reverse transcriptase consensus amino acid |
| | sequences for most of the phylogenetic clades identified in |
| | Table A (sequence included in this Specification) |
| Sequences disclosed | Table X - sequences disclosed in Mestre et al., Systematic |
| in reference | Prediction of Genes Functionally Associated with Bacterial |
| | Retrons and Classification of The Encoded Tripartite |
| | Systems, Nucleic Acids Research, Volume 48, Issue 22, 16 |
| | Dec. 2020, Pages 12632-12647, which is incorporated |
| | herein by reference in its entirety. |
| 19399-19401, 19420, | NLS sequences |
| 19529-19542 | |
| 19402-19415 | TALE related sequences |
| 19416 | Meganuclease |
| 19417 | RE cut site |
| 19418-19419, 19469-19528 | Linkers |
| 19421 | Kozak sequence |
| 19422-19453 | Table Y and related disclosure - exemplary nucleotide |
| | sequences of 5′ UTRs |
| 19454-19468 | Table Z - additional stop elements of linear mRNA |
| 19543-19926 | Table 31A - optimized ncRNA library |
| 19927-19930 | RTX_6342 ncRNAs |

### Retron Sequences

The instant Specification discloses and claims recombinant retrons and components thereof (e.g., recombinant ncRNAs and recombinant retron RTs), as well as genome modification systems comprising said recombinant retrons and/or recombinant components thereof. Without limitation, such systems include recombinant retron-based genome editing systems, recombineering systems, and cell-recording systems. The Specification also discloses and claims the various components and aspects making up the systems, as well as their uses and applications, including without limitation: (a) recombinant nucleic acid molecules encoding recombinant retrons and retron-based genome modification systems, (b) vector systems (including viral and non-viral) comprising one or more components of said retron-based genome modification systems, including all-DNA vector systems, all-RNA vector systems, and DNA/RNA vector systems, (c) delivery systems for delivering said vector systems and/or components of the retron-based genome modification systems (e.g., lipid particles, lipid nanoparticles, and other delivery vehicle formats), (d) formulations comprising any of the aforementioned components for delivery to cells and/or tissues, including in vitro, in vivo, and ex vivo delivery, (e) cells modified by the recombinant retron-based genome modification systems and methods described herein, and (f) methods of modifying cells by conducting genome editing, recombineering, or cell recording using the herein disclosed retron-based genome modification systems, (g) methods of making the recombinant retrons, retron-based genome modification systems, vectors, and formulations described herein, and (h) pharmaceutical compositions and kits for modifying cells under in vitro, in vivo, and ex vivo conditions.

The recombinant retrons and retron-based genome modification systems described herein can be made, in various embodiments, by introducing one or more modifications into a known retron, e.g., a retron that is publicly known or previous published (e.g., Table X) or novel retron sequences (Tables A and B, as identified in the Examples).

### Table X: Previously Known Retron Reverse Transcriptases

Table X provides non-limiting examples of 1928 retron reverse transcriptases that may be modified in accordance with the herein methods to obtain a recombinant retron reverse transcriptase for use in the compositions, systems, and methods described herein. These retron sequences were reported in Mestre et al., “Systematic Prediction of Genes Functionally Associated with Bacterial Retrons and Classification of The Encoded Tripartite Systems,” Nucleic Acids Research, Volume 48, Issue 22, 16 Dec. 2020, Pages 12632-12647, the contents of which are incorporated herein by reference. In some embodiments, the Table X retrons are meant to be excluded from the scope of the claimed subject matter.

Table A with Rows A1-A45: Novel Retron Reverse Transcriptase Sequences

Table A provides non-limiting examples of retron reverse transcriptases that may be modified in accordance with the herein methods to obtain a recombinant retron reverse transcriptase or use in the compositions, systems, and methods described herein.

In particular, Table A provides sequence identifiers corresponding to the novel retron RTs identified as a result of the computational discovery work described in the Examples. The table provides sequence identifiers corresponding to the contents of the Sequence Listing included as part of this Specification. The table includes both RT amino acid sequences and RT nucleic acid sequences. The table is organized into forty-five rows each of which represents those sequences forming a single phylogenetic clade of related retron RTs as determined by the computational work described in the Example 3.

| | Seq ID Nos (sequences presented in the Sequence Listing) | | Phylogenetic |
|---|---|---|---|
| Table | RT Amino Acid Sequence | RT Nucleic Acid Sequence | Clade |
| A1 | 3980-4178 | 11231-11429 | I-A |
| A2 | 4671-4825 | 11922-12075 | I-B1 |
| A3 | 4980-5143 | 12229-12392 | I-B2 |
| A4 | 367-368, 427-441, 494- | 7624-7625, 7684-7698, 7751- | I-C |
| | 521, 526-527, 536, 626, | 7778, 7783-7784, 7793, 7883, | |
| | 649, 660-668, 675, 679, | 7906, 7917-7925, 7932, 7936, | |
| | 687-692, 695, 697, 703, | 7944-7949, 7952, 7954, 7960, | |
| | 716, 721-722, 751-763, | 7973, 7978-7979, 8008-8020, | |
| | 767, 770-1411, 1456-1462 | 8024, 8027-8667, 8712-8718 | |
| A5 | 1529-1569 | 8784-8823 | I-D |
| A6 | 6697-6701 | 13943-13947 | II |
| A7 | 4179-4670 | 11430-11921 | II-A1 |
| A8 | 4884-4909 | 12134-12159 | II-A1 other |
| A9 | 6919-6972 | 14163-14215 | II-A2 |
| A10 | 2786-2866, 2887-2938 | 10039-10119, 10140-10191 | II-A3 |
| A11 | 4826-4863 | 12076-12113 | II-A4 |
| A12 | 4864-4875 | 12114-12125 | II-A4 fused |
| A13 | 6974-7002 | 14217-14244 | II-A5 |
| A14 | 2598-2600, 2759-2785 | 9851-9853, 10012-10038 | III |
| A15 | 2445-2582 | 9699-9836 | III-A1 |
| A16 | 1983-2158 | 9237-9412 | III-A2 |
| A17 | 1612-1982 | 8866-9236 | III-A3 |
| A18 | 2601-2678 | 9854-9931 | III-A4 |
| A19 | 2679-2758 | 9932-10011 | III-A5 |
| A20 | 3442-3603 | 10694-10855 | IV |
| A21 | 3604-3708 | 10856-10959 | V |
| A22 | 2939-3441, 3709-3979, | 10192-10693, 10960-11230, | VI |
| | 5177-5192 | 12426-12441 | |
| A23 | 7003-7033 | 14245-14275 | VII-A1 |
| A24 | 7054-7133 | 14296-14374 | VII-A2 |
| A25 | 7034-7049 | 14276-14291 | VIII |
| A26 | 6835-6918 | 14079-14162 | IX |
| A27 | 6823-6834 | 14068-14078 | X |
| A28 | 298-366, 369-373, 442- | 7555-7623, 2626-7630, 7699- | XI |
| | 493, 522-525, 528-535, | 7750, 7785-7792, 7794, 7808- | |
| | 537, 551-554, 557, 560- | 7811, 7814, 7817-7882, 7929- | |
| | 625, 672-674, 680-681, | 7931, 7937-7938, 7941-7943, | |
| | 684-686, 696, 698, 702, | 7953, 7955, 7959, 7980-7999, | |
| | 723-742, 764-766, 1412- | 8021-8023, 8668-8706, 8708- | |
| | 1450, 1452-1453, 1463- | 8709, 8719-8722, 8825-8831 | |
| | 1466, 1571-1577 | | |
| A29 | 374-426, 539-550, 555- | 7631-7683, 7796-7807, 7812- | XII |
| | 556, 558-559, 671, 682- | 7813, 7815-7816, 7928, 7939- | |
| | 683, 743, 745-750 | 7940, 800, 8002-8007 | |
| A30 | 5942-6665 | 13189-13911 | XIII |
| A31 | 1-297, 715, 1580-1603 | 7258-7554, 7972, 8834-8857 | XIV |
| A32 | 705-714 | 7962-7971 | XV |
| A33 | 6681-6694 | 13927-13940 | XVI |
| A34 | 6788-6803 | 14033-14048 | XVII |
| A35 | 1469-1526, 5147-5151 | 8725-8781, 12396-12400 | CRISPR- |
| | | | associated |
| A36 | 2159-2428 | 9413-9682 | Ec107-like |
| A37 | 646-648 | 7903-7905 | RT-atpase |
| A38 | 2592-2595 | 9846-9849 | RT-DUF4116 |
| A39 | 676-678, 717-720 | 7933-7935, 7974-7977 | RT-HTH |
| A40 | 538, 669, 704, 1454 | 7795, 7926, 7961, 8710 | RT-pddex |
| A41 | 670, 699-701 | 7927, 7956-7958 | RT-unk |
| A42 | 4917-4979 | 12167-12228 | Phage |
| A43 | 4910-4916 | 12160-12166 | Jumbophage |
| A44 | 5195-5941 | 12444-13188 | Outgroup |
| A45 | 627-645, 650-659, 693- | 7884-7902, 7907-7916, 7950- | unclassified |
| | 694, 744, 768-769, | 7951, 8001, 8025-8026, 8707, | |
| | 1451, 1455, 1467-1468, | 8711, 8723-8724, 8782-8783, | |
| | 1527-1528, 1570, 1578, | 8824, 8832-8833, 8858-8865, | |
| | 1579, 1604-1611, 2429- | 9683-9698, 9837-9845, 9850, | |
| | 2444, 2583-2591, 2596- | 10120-10139, 12126-12133, | |
| | 2597, 2867-2886, 4876- | 12393-12395, 12401-12425, | |
| | 4883, 5144-5146, 5152- | 12442-12443, 13912-13926, | |
| | 5176, 5193-5194, 6666- | 13941-13942, 13948-14032, | |
| | 6680, 6695-6696, 6702- | 14049-14067, 14216, 14292- | |
| | 6787, 6804-6822, 6973, | 14295, 14375-14498 | |
| | 7050-7053, 7134-7257 | | |

Table B with Rows B1-B45: Exemplary ncRNA Sequences

Table B provides non-limiting examples of retron ncRNA sequences that may be modified in accordance with the herein methods to obtain a recombinant retron ncRNA sequences for use in the compositions, systems, and methods described herein. The sequences were discovered by the method of Example 3.

In particular, Table B provides sequence identifiers corresponding to the novel retron RTs identified as a result of the computational discovery work described in the Examples. The table provides sequence identifiers corresponding to the contents of the Sequence Listing included as part of this Specification. The table is organized into forty-five rows each of which represent those sequences forming a single phylogenetic clade of related retron ncRNAs as determined by the computational work described in the Example 3.

In various embodiments and in the claims, the disclosure provides recombinant retron-based genome editing systems which comprise combining in a cell through various delivery strategies a retron RT together with an ncDNA. In various aspects, the retron RTs and the ncDNA constituting the recombinant retron-based genome editing system can be based on pairing together such components that are naturally found associated to one another in nature, i.e., sourced from the same bacterial species. These are referred to as the “cognate” pairings of retron RT and retron ncRNA. A cognate pairing of a ncRNA and an RT (amino acid or nucleotide sequence) can easily be identified from information presented/included in the Sequence Listing by identifying those ncRNA sequences from Table B and those RT sequences from Table A have the same genomic accession number that is indicated for each sequence in the Sequence Listing. In various other aspects, the retron RT component and the ncRNA component can be from different bacterial species, i.e., are not found together in nature as cognate pairs. In still other embodiments, the retron RT component and the ncRNA component can both be from the same phylogenetic functional type (e.g., Type I-A, Type I-B1, Type I-B2, Type IC, etc.). For example, a recombinant retron-based genome editing system may be comprised of a retron RT from Type I-A (i.e., SEQ ID Nos: 3980-4178 for AA and SEQ TD Nos: 11231-11429 for NT-see Table A) and a retron ncRNA also from Type I-A (i.e., SEQ TD Nos: 16886-17078—see Table B).

| | Seq ID Nos (sequences presented | Phylogenetic |
|---|---|---|
| Table | in the Sequence Listing) | Clade |
| B1 | 16886-17078 | I-A |
| B2 | 17478-17622 | I-B1 |
| B3 | 17677-17756 | I-B2 |
| B4 | 14831-14833, 14838, 14847, 14850-15460 | IC |
| B5 | N/A | 1D |
| B6 | N/A | II |
| B7 | 17079-17477 | II-A1 |
| B8 | 17660-17676 | II-A1 other |
| B9 | 19031-19080 | II-A2 |
| B10 | 16414-16516 | II-A3 |
| B11 | 17623-17659 | II-A4 |
| B12 | N/A | II-A4 fused |
| B13 | 19081-19108 | II-A5 |
| B14 | 16397-16413 | III |
| B15 | 16195-16320 | III-A1 |
| B16 | 15779-15925 | III-A2 |
| B17 | 15476-15778 | III-A3 |
| B18 | 16321-16366 | III-A4 |
| B19 | 16367-16396 | III-A5 |
| B20 | 16705-16814 | IV |
| B21 | 16815-16885 | V |
| B22 | 16517-16704 | VI |
| B23 | N/A | VII-A1 |
| B24 | N/A | VII-A2 |
| B25 | N/A | VIII |
| B26 | 18949-19030 | IX |
| B27 | N/A | X |
| B28 | 14657-14716, 14778-14824, 14834, | XI |
| | 14835-14836, 14839, 15461-15475 | |
| B29 | 14717-14777, 14841-14846 | XII |
| B30 | 18413-18936 | XIII |
| B31 | 14499-14656 | XIV |
| B32 | N/A | XV |
| B33 | 18939 | XVI |
| B34 | N/A | XVII |
| B35 | N/A | CRISPR- |
| | | associated |
| B36 | 15926-16178 | Ec107-like |
| B37 | N/A | RT-atpase |
| B38 | N/A | RT-DUF4116 |
| B39 | N/A | RT-pddex |
| B40 | N/A | RT-HTH |
| B41 | 14837 | RT-unk |
| B42 | N/A | Phage |
| B43 | N/A | Jumbophage |
| B44 | 17757-18412 | Outgroup |
| B45 | 14825-14830, 14840, 14848-14849, | unclassified |
| | 16179-16194, 18937-18938, 18940-18948 | |

### Table C: Consensus RT Amino Acid Sequence

Table C provides a consensus amino acid sequence for most of the phylogenetic types of RT identified in Table A.

| RT Type | | |
|---|---|---|
| (based | | |
| on Phylo- | | SEQ |
| genetic | | ID |
| Clade) | Consensus Amino Acid Sequence | NO: |
| TypeIA | YKVYXIPKRXXGXRXIAXPXXXLKXXQXXX | 19365 |
| | XXXXXXXXXXHXXXXAYXXXXXIKXNAXXH | |
| | XXXXYXLKXDXXXFFNSIXXXXXXXXXXXX | |
| | XXXXXXXXXXXXXXXXFWXXXXXXXXXLXL | |
| | SXGAPSSPXXSNXXMXXFDXXXXXXCXXXX | |
| | XXYXRYADDXTFSTXXXXXLXXXPXXXXXX | |
| | LXXXXXXXXXXNXXKTXFSSKAHNRHXTGX | |
| | TXXNXXXXSXGRXXKRXIXXLXXXXXXX | |
| TypeIB1 | XXXXXXXXXXXXXXXXXLKXXXXFXXXXXX | 19366 |
| | XXXXXXXXXVXSYRKGXXXXXAVXXHXXXX | |
| | XFXXXDXXXFFXSIXXXXXXXXXXXXXXXX | |
| | PXXDXXXXXXXXXXXXXXXXXLPXGXXTSP | |
| | XXSNXXLXXFDXXXXXXCXXXXXXYTRYXD | |
| | DXIXSXXXXXXXXXXXXXXXXXLXXXXXXX | |
| | XXXNXXKXKXXXXGXXXKXLGXXILPXGXX | |
| | XXXXXXKXXXEXXXXXXXXX | |
| TypeIB2 | XXXXXXXXXXXXXXXXXXXXXXXXXXXXXX | 19367 |
| | XXXXXXXXXXXXXXXXXXXXXSYXXXXXXH | |
| | XXXXXXXRXDIXXFFXSIXXXXXXXXXXXX | |
| | XXXXXXXXXXXXXXXXXXXXXXXXXXXXXX | |
| | XXXXPXGXXXSPXXSNXXFRXXDXXIXXXC | |
| | XXXXXXYXRYADDXLFSXXXXXXXXXXXXF | |
| | XXXIXXXXXXXXXXXNXXKXXXXXXXXSLN | |
| | GXXXXXXXXXXXXXXXXXXXXXXXXXXXXX | |
| | X | |
| TypeIC | YXXFXIXKXXGXXRXIXAPXXXLKXXQXXL | 19368 |
| | XXXLXXXXXXXXXXXXXXXXXXHXFXXXXX | |
| | IXXNAXXHXXXXXVXNXDLXXFFXXXXFGR | |
| | VXGXFXXXXXFXXXXXXAXXXAQXXCXXXX | |
| | LPQGXPXSPXIXNXIXXXLDXXXXXXXXXX | |
| | XXYXRYADDXTFSTXXXXXXXXXXXXXXXX | |
| | XXXXXXXXXXXXXXGFXXNXXKTRXXXXXX | |
| | RQXVTGLXVNXXXNXXXXYXXXXRXXXXXX | |
| | XXX | |
| TypeID | YXXXXXXKKXGGXRXIXXPXXXLXXXQXXL | 19369 |
| | XXXLXXXYXXXXXXXXXGFXXXXXXXXXXX | |
| | XIXXNAXXIIXXKXXXLNXDXXXFFXSIXX | |
| | XXXXXXXXXXXFXXXXXXAXXXXLLXTXXX | |
| | XLPXGAPXSPXXSNXXCXXXDXXLXXXXXX | |
| | XXXXYXRYADDLTFSXXXXXXXXXXXXXXX | |
| | XIXXXXFXXNXKKXRXXXXXXXQXVTGXXV | |
| | NXKXNXXRXXXXXXRAXXHXXXXX | |
| TypeIIAl | YKXXXIXKXXGXXRXIXXPXXXXKXXQXXX | 19370 |
| | XXXXXXXXXXHXXAXAYXXXXXIXXNAXXH | |
| | XXXXXXXXXDFXXFFXSIXXXXXXXXXXXX | |
| | XXXXXXXXXXXXXXXXXXXXXXXXXXXXLX | |
| | IGXPXSPXXSNXXXXXXDXXXXXXXXXXXX | |
| | XYXRYADDXXXSXXXXXXXXXXXXXXXXXX | |
| | XXXXXXXXXXNXXKTXXXXXXXXXXXTGXX | |
| | XXXXXXXXXGRXXKRXXXXXXXXXXXX | |
| TypeID | YXXXXXXKKXGGXRXIXXPXXXLXXXQXXL | 19371 |
| | XXXLXXXYXXXXXXXXXGFXXXXXXXXXXX | |
| | XIXXNAXXHXXKXXXLNXDXXXFFXSIXXX | |
| | XXXXXXXXXXFXXXXXXAXXXXLLXTXXXX | |
| | LPXGAPXSPXXSNXXCXXXDXXLXXXXXXX | |
| | XXXYXRYADDLTFSXXXXXXXXXXXXXXXX | |
| | IXXXXFXXNXKKXRXXXXXXXQXVTGXXVN | |
| | XKXNXXRXXXXXXRAXXHXXXXX | |
| TypeIIA1 | YXXXXXXXXXXXXXXXXPXXXLKXXQXWXX | 19372 |
| | XXXXXXXXXXXXXXAYXXXXSXXXXAXXHX | |
| | XXXXXXXXDIXXFFXSXXXXXXXXXXXXXX | |
| | XXXXXXXXXXXXXXXXXXXXXXLXXGXXXS | |
| | PXIXNXXMXXXDXXXXXXXXXXXXXYXRYX | |
| | DDIXXSSXXXIXXXXXXXXXXXLXXXXXXX | |
| | NXXKTXXXXXXXXXXXTGXXXXXXXXXXGX | |
| | XXXXXXXXXXXXXXXX | |
| TypeIIA2 | YRXFXXXKXXGXXRXIXXPRXFXKXXQXXX | 19373 |
| | XDXXLXXLXXHXXXXXXXXXXSXXXNAXXH | |
| | XXXXXXXXXDIXXXFXXIXXXXXXXXXXXX | |
| | XXXXXXXXXXXXXXTXXXXLPQGAPTSPXX | |
| | SNXXLXXFDXXXXXXXXXXXXXYXRYXDDX | |
| | TXSXXXXXXXXXXXXXXXXXLXXXXXXXNX | |
| | XKXRXXXXXXXQXVTGXXXNXXXXPXRXXR | |
| | XXXRAXXXXAXX | |
| TypeIIA3 | YXXXXXXXXXXXXRXIXXPXXXLKXXQXWI | 19374 |
| | LXXILXXXXXSXXXXXFXXXXXXXXNAXXH | |
| | XXXXXXLXXDXXXFFXXXXXXXVXXXFXXX | |
| | GYXXXXXXXLXXXCXXXXXLPQGXXXSPXX | |
| | XNLXXXXLDXRXXXXXXXXXXXYTRYADDX | |
| | XXSXXXXXXXXXXXXXXXXIXXXEXXXXNX | |
| | XKXXXXXXXXXXXXTGXXXXXXXXXXXXXX | |
| | XXXRXXXXXXXX | |
| TypeIIA4 | XXXXXIXXXXXXRKIXTXXXXXXXXXXXXX | 19375 |
| | XXXXXXXXXXXXXFXKAYXXXXSIXXNAXX | |
| | HMYNDXFXXXDIXXFFXXIXHXXLXXXLXX | |
| | XXXXXXXXXXXXXXXXXXXXXXXXXXXGLX | |
| | XGXXXSPXLXNXYXKXFDXIXYGXLKXXXX | |
| | XXXIYTRYADDXXISFKXXXXXXXXXXXIX | |
| | XXXXXXLXXXXLXXNXXKXXXXXXXXSNHV | |
| | XITGXXIXXXXXXXRXXXVGXXXXXXLXXX | |
| | AXXXXXX | |
| TypeIIA4 | XXXXXXXXXXKXRXXXXXXXXXXGXXXXXX | 19376 |
| | HXXXXXXXXXXXXXXXXSYAYXXXXSIXXC | |
| | XXXHXXXXXFXKXDIXXFFNSIXXXXLXXX | |
| | XXXXXXXXXXXXXXXXXXXXXXXXXXXXPX | |
| | GLXXSPXXSDXYXXXXXXXXXXXXXXXXXX | |
| | YTRYADDIXISXXXXXXXXXXXXIXXXXXX | |
| | XLXXXXLXXNXXKXXXXXXXXXXXXXXXXG | |
| | XNIXXXXXXXXXXVGXXXXXXXXXXXXXXX | |
| | XX | |
| TypeIIA5 | YRXFXXXKXXGXXRXIXXPXTYLKVXQWWI | 19377 |
| | XDXIXXXXXXXXXXXGFXXGXXXXXNAXXH | |
| | XXXXXXLNXDXXXFFXSXXXXXXXXXFXXX | |
| | GXXXXXXXXLXXLXXXXXXXPXGAPTSPXX | |
| | XNXXXXXXDXXLXXXXXXXXXXYXRYADDX | |
| | TFSXXXXXXXXXXXXXXXXXXXXGFXXXXX | |
| | KTXXXGXXXRXXVTGXXXNXXXXXXXXXRX | |
| | XXRXXXHXXXXX | |
| TypeIIIA1 | YRXXXIXKXXGXXRXIXEPLPXLKXIQXWI | 19378 |
| | LXXILXXXXXSXXAKAXXXXXXXXXNXXXH | |
| | XXXXXXXXXDXXXFFXXIXXXXXXXXFXXX | |
| | GYXXXXXXXLXXLCXXXXXLPQGAPTSPXL | |
| | SNXXXXXXDXXXXXXXXXXXXXYTRYADDX | |
| | XFSGXXXXXXXXXXXXXXXXXXXXXXNXXK | |
| | XXXXXXXXXQXVTGXVVNXKXQXXXXXRXX | |
| | XRXXXXXIXK | |
| TypeIIIA2 | YRXFXIXKXXGGXRXIXXPXXXLXXXQXXI | 19379 |
| | XXXILXXXXXXXXXXXXXXXXSXXXNAXXH | |
| | XXXXXXLKXDXXXFFXSIXXXXXXXXFXXX | |
| | GYXXXXXXXLXXXCXXXXXLPQGAXTSPXL | |
| | SNXXXXXXDXXLXXXXXXXXXXYXRYADDX | |
| | XXSGXXXXXXXXXXXXXXXXXXGXXXNXXK | |
| | XXXXXXXXXXIXTGXXXXXXXXXXPXXXXR | |
| | XXXXXXXXXXXX | |
| TypeIIIA3 | YXXXXXXXXXXXXRXIXXPXXXLXXXQXXI | 19380 |
| | XXXXLXXXXXHXXXXAXXXXXXXXXXAXXH | |
| | XXXXWXXKXDXXXFFXXXXEXXXXXXFXXX | |
| | GYXXLXXXEXARXCTXXXXXXXXXXXXXXX | |
| | XXXXXXXXXXXXGXLPQGAPTSXXLXNLXX | |
| | XXXDXXXXXXAXXXXXXYTRYXDDXXXSXX | |
| | XXXXXXXXXXXXXXXXXXXXXXGXXXXXXX | |
| | XXXXXPGXXXXVLGLXVXXXXXXLXXXXXX | |
| | XXXXHXXXXXXX | |
| TypeIIIA4 | YXXXXXXKXXGGXRXIXXPXXXLXXXQXWI | 19381 |
| | XXNILXXXXXXXXXXGFXXXXSIXXNAXXH | |
| | XXXXXXLXXDLXXFXXXIXXXXXXXXFXXX | |
| | GYXXXXXXXXAXXXTXXXXXXXXXXXXXXX | |
| | XXXXXLPQGAPXSPXXXNXXXXXXDXRXXX | |
| | XXXXXXXXYXRYADDXTFSXXXXXXXXXXX | |
| | XXXIXXXEXXXXNXXKXXXXXXXXXXXVTG | |
| | LXXXXXXXXXXXXXXXXXXXXXXCXK | |
| TypeIIIA5 | YXXXXIXKXXGXXRXXXXPXXXLKXXQXWI | 19382 |
| | LXXILXXXXXXXXXXGFXXXXSIXXNAXXH | |
| | XXXXXXXXXDXXXFFPXIXXXXXXXXFXXX | |
| | GYXXXXXXXXXXXCTXXXXLPQGXPXSPXX | |
| | XNXXXXXXDXRXXXXXXXXXXXYXRYADDX | |
| | TXSGXXXXXXXXXXXXXIXXXXXXXXNXXK | |
| | XXXXXXXXXQXVTGXXVNXXXXXXXXXXXX | |
| | XXXXIYXXXKX | |
| TypeIIIunk | YXXXXXXXXXXXXXXXXPXXXLKXXQXWIX | 19383 |
| | XNILXXXXXXXXXXXXXXXXSIXXNAXXHX | |
| | XXXXXXXXDIXXFFXSIXXXXXXXXFXXXX | |
| | XXXXXXXXXXXXXXXXXXXXQGXPXSPXXX | |
| | NXXXXXXDXXXXXXXXXXXXXYXRYADDXX | |
| | XSXXXXXXXXXXXXXXXXXXXXXXXXNXXK | |
| | TXXXXXXXXXXXTGXXXXXXXXXXXXXXXX | |
| | XXXEXXYCXX | |
| TypeIV | YXXXXXXKXXGXXRXXXXPXXXXRXXQXRI | 19384 |
| | NXRIFXXXXXWPXXXXGSXPXXXXXXXXXX | |
| | XDYXXCAXXHCXXKXXLKXDIXXFFXNXXX | |
| | XXVXXXFXXXXXXXXXXXXXXXXCXXXXXX | |
| | XQGXXTSSYXAXLXLXXXEXXXXXXXXXKX | |
| | LXYTRXVDDITXSSXXXXXXFXXXXXXXXX | |
| | MLXXXXLPXNXXKXXXXXXXXXXLXVHGLR | |
| | XXXXXPRXPXXEXXXIRXXVXXXXXX | |
| TypeV | YXXXXXXXXXXXKXRXXXXPXXXLKXXQKR | 19385 |
| | INXXIFXXXXXPXYLXGGXXXXXXXRDYXX | |
| | NXXXHXXXXXXIXLDXXXFYXXIXXXXVXX | |
| | XXXXXXXFXXXVXXXLXXLXTXXXXXPQGX | |
| | CTSSYXANLXXXXXEYXXXXXXXXXXXXYX | |
| | RLLDDXTXSXXXXXXXXXXXXXXXXXXXXX | |
| | XXXLXXXXXXXXXXXXXXXXXXXXVTGLWX | |
| | XXXXPXXXXXXRXXIRXXVXXCXXX | |
| TypeVI | XXXXXXXXXXXXXRXXXXXXXXLXXXXXXX | 19386 |
| | XXXXXXXXXPXXXXXXXXXXXXXXNAXXHX | |
| | XXXXXXXXDXXXFXXXXXXXXXXXXXXXXX | |
| | XXXXXXXXXXXXXXXXXXXXXXGXXXSXXX | |
| | XXXXXXXXXXXXXXXXXXXXXXXXXXXDDX | |
| | XXSXXXXXXXXXXXXXXXXXXXXXXXXXXX | |
| | KXXXXXXXXXXXXTGXXXXXXXXXXXXXXX | |
| | XXXXXXXXXXXXX | |
| TypeVIIA1 | XXXXXXXXXXXXXXXRXVWEXXXXXXXXXK | 19387 |
| | XXXRXXXXFXXXXXXXXPHXXXXGYXXGRX | |
| | XRXNAXXHXGXXXXXXXDXXXFFPSIXXXR | |
| | XXXXXXXXGXXXXXXXXLXXFXTIXXXLPL | |
| | GLXXSPXXXNXXXXXXDXXLXXLAXXXXXX | |
| | YXRYXDDXXXSXXXXXPXXXXXXXXXXXXX | |
| | FXXXXXKXXXSKXGQXHXVTGLSXXXXXXP | |
| | HXPRXXKXXLRQELXXXXXX | |
| TypeVIIA2 | XXXXXXXXXXXXXRXXXXXXXXXXXXXKXX | 19388 |
| | XXXXXXXXXXXXXXGFXXXXXXXXNAXXHX | |
| | XXXXXXXXDXXXFFXXIXXXXXXXXXXXXX | |
| | XXXXXXXXXXXXXTXXXXLXXGXXTSPXXX | |
| | NXXXXXXDXXXXXXXXXXXXXXRYXDDXXF | |
| | SXXXXXXXXXXXXXXXXXXXXXXXXNXXKX | |
| | XXXXXGXXQXVTGLXXXXXXXPRXXXXXKX | |
| | XXRXXXXXXXX | |
| TypeVIII | YXXXXXXKRXXXXXGEXRXVXXAXXXXXXX | 19389 |
| | XHRXXXXXXXXXXXFGXHVQGFXXXRSXXX | |
| | NAXXHXXXXXXXHADIXXFFXXITXXQVXX | |
| | XXXXXXXXXXXAXXXAXXCTIDGXLXQGTR | |
| | CSPXXXNXVCXXXDXXXLXLAXXXXXXXXR | |
| | YADXXTFSGXXXXXXXXXXXXXXXXGFXLR | |
| | XXXCYXQXXGXXQXVTGLXVXDXXXPRLPK | |
| | XXKXXLRLXXXXXXKX | |
| TypeIX | YRXFXIXXXXXXXRXIXAPXVXLKXXQXWX | 19390 |
| | XXXXXXXXXXXXXVXGFXXGXXXXXAAXXH | |
| | XXAXWXXSXDXXXFFXXXXXXXXXXXLXXX | |
| | GYXXXXXXXXXXXXXXXXXLXQGXPXSPXX | |
| | SNXXXXXXDXXXXXXXXXXXXXXXRYADDX | |
| | XFSGXXXXXXXXXXXXXXXXXXXXWXXXXX | |
| | XXXXXXXPXRLKVHGLLVXXXXXXLTKGYR | |
| | NXXRAXXHXXXXX | |
| TypeX | YXXXPXXXXXXXXRWIEAPXXXLKXXQRXX | 19391 |
| | LXXXXYXXXXXXXAHGFXXGRSIXXNAXXH | |
| | XGXXXVVXXDXXXFFPXXXXXXXXXXXXXX | |
| | XXXXXXXXXXXXLXXXXXXLPQGAPTSPXL | |
| | XNLVXXXXDXXLXXXAXXXXXXYTRYADDL | |
| | XFSXXXXXXXXXXXXXXXXXXIXXXXGXXX | |
| | XXXKXXXXXXXQRQXVTGXVVNXXXXLPXX | |
| | XRRXLRAXXXXXXXX | |
| TypeXI | YXXFXIXKXXGXXRXIXXPXXXLKXXQXXX | 19392 |
| | XXXLXXXXXXXXXXXGFXXXXXXXXNAXXH | |
| | XXXXXXXNXDLXXFFXXIXXXRXXXXXXXX | |
| | XXXXXXXXAXXXAXXXXXXXXLPQGAPXSP | |
| | XXSNXXXXXXDXXLXXXAXXXXXXYTRYAD | |
| | DXTXSXXXXXXXXXXXXXXXXXXXXXXXXX | |
| | XXXXXXXXFXXNXXKXXXXXXXXXXXVTGX | |
| | XXNXXXNXXRXXXXXXXXXXXXXXXX | |
| TypeXII | YXXFXXXKXXGGXRXIXAPXXXLXXXQXXX | 19393 |
| | XXXXXXXXXXXXXAHGFXXXXSXXXNAXXH | |
| | XXXXXXXXXDXXXFFPXXXXXRVXGXFXXX | |
| | GYXXXXAXXXAXXXTXXXXXXXXXXXXXXX | |
| | XXXXXRXLPQGAXXSPXXXNXXXXXLDXRL | |
| | XXXAXXXXXXYTRYADDXTFSXXXXXXXXX | |
| | XXXXXXXXXXXXXEGFXXXXXKXXXXXXXX | |
| | XQXVTGXXVNXXXXXXRXXXXXXRAXXXXX | |
| | XXX | |
| TypeXIII | YXXFXXPKXXGGXRXIXAPXXXLXXXQXXX | 19394 |
| | LXXXXXXXXXXXXAIIGFXXXXSXXXNAXX | |
| | IIXXXXXXXXXDXXXFFPXXXXXRVXGXFX | |
| | XXGYXXXXAXXXXLXXTXXXXXXXXXXXXX | |
| | XXXXXXXRXLPQGAXXSPXXXNXXXXXLDX | |
| | RLXXXAXXXGXXYTRYADDLTFSXXXXXXX | |
| | XXXXXXXXXXXXXXXEGFXXXXXKXXXXXX | |
| | XXQXVTGXVVNXXXXXXRXXXXXXRAXXXX | |
| | XXXX | |
| TypeXIV | YXXFXIXKKXGXXRXIXXPXXXLXXXXXXX | 19395 |
| | XXXXXXXXXXXXXXXGFXXXXSXXXNAXXH | |
| | XXXXXVXNXDLXDFFXSXXXXRXXXXXXXX | |
| | PXXXXXXXXAXXXXXLCXXXXXXXXXXXXX | |
| | LPQGXPXSPXXXNXXCXXLDXXLXXXAXXX | |
| | XXXYXRYADDXTFSXXXXXXXXXXXXXXXX | |
| | XXXXXXXXXNXXKTRXXXXXXRQEVTGXXV | |
| | XXXXNVXXXYXXXXRXXLXXWXXX | |
| TypeXV | YXXFXXXKKSGGXRXIXXPXKSLXIXQXKL | 19396 |
| | SQXLYXXYXPXXXVHGXXXXXSIXTNAXXH | |
| | XXKXFXLNXDIXDFFXSINXGRVRGXFIAX | |
| | PYXLXXXVATXXAXICCXXNKLPQGAPXSP | |
| | IXSNLICXXXDXELQXFAXXXXXXYTRYAD | |
| | DITXSXXXXXLPXXLXXXXXXXXXXXXLGX | |
| | ELXXIIXXNGFXINXXKXRLXYXXQXQXVT | |
| | GLXVNXXVNVXRKYIRNXXXXLHAWEKX | |
| TypeXVI | YXXFXXXKXXGXXRXIXAPXXXLKXXQXXI | 19397 |
| | LXXXLXXVXLXXXAXGFRXXRSIXTNAXXH | |
| | XXXXXXXKXDXKXFFPSXXXXRVXGXXXXL | |
| | GYPXXXXXXLTXLXTXXXXLPXGAPTSPXX | |
| | XNXXXXRXDXRXXXLXXKXXFXYSRYADDX | |
| | XXSSXXXXXXXXIPFFXXIXXXEGFXXNEX | |
| | KXXIXRXGXRQXXTGXVVNXKXNXXXXEXX | |
| | XLRAVXXNCXXX | |
| TypeXVII | YRXFXXXKXDGXXRXXXXPXXXLKXXQXXX | 19398 |
| | XXXXLXXXXXHPXAXXFXXXXSXXXXAXXH | |
| | AXXXXXXTXDXXDFFXXTXXXRVXXXXXXX | |
| | XXXXXXXXXLXXLXXXXXXLPQGAPTSPXL | |
| | SNXVNXXXDXXXXXXXXXXXXXYTRYXDDX | |
| | XFSWXXXXXPXXFXXXXXXXLXXXGYXXXP | |
| | XKXXXXXXXXXXXPXXTGXXLXXXGXXXXP | |
| | XXXXXXXXXXXX | |

Table X—Previously known retron RTs (Accession No.: Organism)

The following sequences are disclosed in Mestre et al., Systematic Prediction of Genes Functionally Associated with Bacterial Retrons and Classification of The Encoded Tripartite Systems, Nucleic Acids Research, Volume 48, Issue 22, 16 Dec. 2020, Pages 12632-12647, which is incorporated herein by reference in its entirety. The sequences are described as sequence reference numbers and the source organism.

Sequence Accession No.; source organism

FIG|670897.3.peg.2382; Escherichia coli 2362-75
WP_000111473.1; Escherichia coli (Retron-Eco7)
FIG|286156.4.peg.5031; Photorhabdus australis
FIG|171439.3.peg.1995; Photorhabdus luminescens subsp. luminescens
FIG|1004151.3.peg.110; Photorhabdus khanii NC19
FIG|1736225.3.peg.2969; Erwinia sp. Leaf53
FIG|1897730.3.peg.2912; Citrobacter sp. CFSAN044567
FIG|286156.4.peg.5031; Aeromonas australiensis
FIG|1460083.3.peg.4429; Serratia liquefaciens FK01
FIG|585.10.peg.2369; Proteus vulgaris
WP_140315795.1; Vibrio parahaemolyticus (Retron-Vpa1)
FIG|670.147.peg.3463; Vibrio parahaemolyticus
FIG|1516159.4.peg.4737; Vibrio coralliirubri
FIG|190893.12.peg.246; Vibrio coralliilyticus
FIG|643674.5.peg.820; Paenalcaligenes hominis
FIG|1122619.3.peg.2381; Oligella ureolytica DSM 18253
FIG|29489.5.peg.3423; Aeromonas enteropelogenes
FIG|1899355.18.peg.3566; Oceanospirillaceae bacterium
FIG|49186.3.peg.4362; Marinobacterium stanieri
FIG|672.375.peg.4377; Vibrio vulnificus
FIG|584.202.peg.1668; Proteus mirabilis
FIG|394935.10.peg.4407; Chromobacterium haemolyticum
FIG|1196083.117.peg.637; Snodgrassella alvi
FIG|1196083.120.peg.2046; Snodgrassella alvi
FIG|1196083.114.peg.825; Snodgrassella alvi
FIG|550.250.peg.2975; Enterobacter cloacae
FIG|680.27.peg.793; Vibrio campbellii
FIG|1348393.3.peg.352; Pseudoalteromonas sp. H105
FIG|1234128.4.peg.4777; Vibrio parahaemolyticus SNUVpS-1
FIG|69219.6.peg.2213; Enterobacter cloacae subsp. dissolvens
FIG|208224.13.peg.2962; Enterobacter kobei
FIG|672.332.peg.2758; Vibrio vulnificus
FIG|1777131.3.peg.2267; Chromobacterium sp. F49
FIG|945550.3.peg.1167; Vibrio sinaloensis DSM 21326
FIG|648.75.peg.922; Aeromonas caviae
FIG|1238221.3.peg.2053; Vibrio parahaemolyticus VPTS-2009
FIG|56192.3.peg.3860; Photobacterium iliopiscarium
FIG|1806667.7.peg.3169; Marinomonas gallaica
FIG|272773.3.peg.1019; Salinivibrio costicola subsp. alcaliphilus
WP_073265166.1; Pseudomonas punonensis
FIG|1946584.3.peg.2789; Halomonas sp. UBA3074
FIG|2030880.3.peg.665; SAR86 cluster bacterium
FIG|80854.14.peg.530; Moritella viscosa
FIG|1902503.3.peg.1072; Marinomonas sp. QM202
FIG|1122212.3.peg.1985; Marinospirillum minutulum DSM 6287
FIG|40576.4.peg.4387; Xenorhabdus bovienii
FIG|287094.3.peg.78; Alteromonas addita
FIG|1805633.3.peg.1469; Acinetobacter sp. SFA
FIG|1945927.3.peg.1017; Acinetobacter sp. UBA1497
FIG|202956.9.peg.1680; Acinetobacter towneri
FIG|1811612.3.peg.155; Moraxellaceae bacterium REDSEA-S32_B1
FIG|573.14330.peg.438; Klebsiella pneumoniae
FIG|470.1294.peg.971; Acinetobacter baumannii
FIG|762966.3.peg.2452; Parasutterella excrementihominis YIT 11859
FIG|470.3514.peg.1550; Acinetobacter baumannii
FIG|470.2538.peg.3022; Acinetobacter baumannii
FIG|48296.130.peg.276; Acinetobacter pittii
FIG|663.91.peg.4688; Vibrio alginolyticus
FIG|296199.3.peg.4813; Vibrio gigantis
FIG|1367490.3.peg.3583; Aliivibrio fischeri ETJB5C
FIG|326537.3.peg.3698; Colwellia polaris
FIG|1175631.4.peg.4191; Pectobacterium wasabiae CFBP 3304
WP_001403504.1; Escherichia coli (Retron-Eco4/Ec83)
FIG|549.21.peg.1734; Pantoea agglomerans
FIG|140100.3.peg.2972; Vibrio cholerae
FIG|693153.4.peg.1176; Vibrio atlanticus
FIG|1238430.3.peg.1911; Vibrio nigripulchritudo AM115
FIG|1123036.3.peg.144; Psychromonas arctica DSM 14288
FIG|173990.3.peg.3319; Rheinheimera pacifica
FIG|1869214.4.peg.3809; Rheinheimera sp.
FIG|1898113.7.peg.1514; Idiomarinaceae bacterium
FIG|29484.39.peg.1876; Yersinia frederiksenii
FIG|1761793.3.peg.274; Marinobacter sp. DSM 26671
FIG|587.48.peg.2666; Providencia rettgeri
FIG|573.4147.peg.1684; Klebsiella pneumoniae
FIG|1263833.3.peg.2872; Serratia marcescens VGH107
FIG|1690502.3.peg.467; Pantoea sp. CFSAN033090
FIG|1029989.3.peg.5037; Salmonella enterica subsp. enterica serovar Agona str. 0292
FIG|211759.3.peg.770; Serratia marcescens
FIG|29483.5.peg.2283; Yersinia aldovae
FIG|1268238.3.peg.3466; Escherichia coli O5:K4(L):H4 str. ATCC 23502
FIG|548.121.peg.2368; Klebsiella aerogenes
FIG|196024.6.peg.1825; Aeromonas dhakensis
FIG|386429.3.peg.3784; Pseudoalteromonas sp. BSi20495
FIG|666.2089.peg.3167; Vibrio cholerae
WP_159353404.1; Vibrio cholerae (Retron-Vch1/Vc95)
FIG|670.362.peg.2186; Vibrio parahaemolyticus
FIG|615.398.peg.1671; Serratia marcescens
FIG|571.188.peg.5401; Klebsiella oxytoca
FIG|1389422.3.peg.2794; Klebsiella pneumoniae LAU-KP1
FIG|1082704.3.peg.1242; Lonsdalea britannica
FIG|1686379.3.peg.3365; Citrobacter sp. MGH104
FIG|83655.55.peg.221; Leclercia adecarboxylata
FIG|550.532.peg.617; Enterobacter cloacae
FIG|349965.6.peg.153; Yersinia intermedia ATCC 29909
FIG|1947028.3.peg.31; Pantoea sp. UBA2708
FIG|29484.34.peg.3725; Yersinia frederiksenii
FIG|314608.4.peg.222; Shewanella benthica KT99
FIG|585.16.peg.3620; Proteus vulgaris
FIG|1117313.3.peg.4128; Pseudoalteromonas arctica A 37-1-2
FIG|1236543.3.peg.1328; Shewanella putrefaciens JCM 20190 = NBRC 3908
FIG|550.520.peg.1818; Enterobacter cloacae
FIG|592316.4.peg.43; Pantoea sp. At-9b
FIG|1903 177.3.peg.4556; Vibrio sp. 10N.261.45.E1
FIG|1435069.3.peg.925; Vibrio tritonius
FIG|666.3258.peg.1211; Vibrio cholerae
FIG|1579504.3.peg.1822; Shewanella sp. ECSMB14102
FIG|727.548.peg.1576; Haemophilus influenzae
EIJ70524.1; Haemophilus parahaemolyticus HK385
FIG|1121935.3.peg.14; Hahella ganghwensis DSM 17046
FIG|400668.8.peg.2509; Marinomonas sp. MWYL1
FIG|1777491.3.peg.1212; Alteromonas sp. Mac1
FIG|2013797.3.peg.1728; Gammaproteobacteria bacterium HGW-Gammaproteobacteria-15
FIG|1008297.7.peg.4158; Salmonella enterica subsp. enterica serovar Typhimurium str. 798
EDM6246721.1; Salmonella enterica subsp. enterica serovar Typhimurium (Retron-Sen2 (St85))
FIG|421.19.peg.3278; Methylomonas methanica
FIG|758.17.peg.102; Rodentibacter pneumotropicus
FIG|726.60.peg.864; Haemophilus haemolyticus
FIG|1035188.3.peg.348; Haemophilus pittmaniae HK 85
FIG|670.79.peg.3738; Vibrio parahaemolyticus
FIG|1481663.12.peg.913; Vibrio metoecus
FIG|1123402.3.peg.611; Thorsellia anophelis DSM 18579
FIG|668.83.peg.3088; Aliivibrio fischeri
FIG|290110.6.peg.2319; Xenorhabdus budapestensis
FIG|568766.10.peg.822; Dickeya sp. NCPPB 3274
FIG|470.4268.peg.2217; Acinetobacter baumannii
FIG|1977881.3.peg.1569; Acinetobacter sp. ANC 4470
FIG|548.171.peg.2395; Klebsiella aerogenes
FIG|584.105.peg.1823; Proteus mirabilis
FIG|1275975.3.peg.1756; Salmonella enterica subsp. enterica serovar Newport str. Henan_3
FIG|615.474.peg.3994; Serratia marcescens
FIG|61647.13.peg.3699; Pluralibacter gergoviae
FIG|549.22.peg.222; Pantoea agglomerans
FIG|991944.3.peg.3216; Vibrio cholerae HE-25
WP_001022871.1; Vibrio cholerae (Retron-Vch2 (Vc81))
FIG|1638949.3.peg.1051; Vibrio sp. ECSMB14106
FIG|73010.3.peg.2815; Aeromonas encheleia
FIG|1444141.3.peg.3893; Escherichia coli 3-373-03_S3_C1
FIG|232.5.peg.1080; Alteromonas sp.
FIG|1175295.3.peg.21; Pseudoalteromonas sp. PAMC 22718
FIG|265726.7.peg.3430; Photobacterium halotolerans
WP_009585554.1; Acinetobacter
FIG|2004649.3.peg.1632; Acinetobacter sp. WCHA29
FIG|1324350.3.peg.2817; Acinetobacter equi
FIG|2048003.3.peg.1682; Alteromonas flava
FIG|571.171.peg.5963; Klebsiella oxytoca
FIG|573.4060.peg.3574; Klebsiellapneumoniae
FIG|1173850.3.peg.2995; Salmonella enterica subsp. enterica serovar Indiana str. ATCC 51959
FIG|1123516.3.peg.1267; Hydrogenovibrio halophilus DSM 15072
FIG|1981674.3.peg.1814; Pseudomonas sp. R9(2017)
FIG|1947311.3.peg.2053; Pseudomonas sp. UBA2684
FIG|1198309.3.peg.4291; Pseudomonas fluorescens ICMP 11288
FIG|715451.3.peg.1743; Alteromonas naphthalenivorans
FIG|316.285.peg.730; Pseudomonas stutzeri
FIG|1190606.3.peg.313; Enterovibrio calviensis 1F-211
WP_009176189.1;
WP_097050713.1; Thalassospira xiamenensis
FIG|1208323.3.peg.893; Celeribacter baekdonensis B30
KZK95863.1; Pseudovibrio sp. Ad46
FIG|101571.310.peg.3956; Burkholderia ubonensis
FIG|1882791.3.peg.1790; Burkholderia sp. CF099
FIG|1736536.3.peg.4809; Variovorax sp. Root434
PIG30812.1; Janthinobacterium sp. 35
FIG|1798244.3.peg.1046; Gallionellales bacterium GWA2_55_18
FIG|1131551.3.peg.1124; Methylotenera sp. 1P/1
FIG|1843082.3.peg.1574; Macromonas sp. BK-30
FIG|279058.16.peg.4721; Collimonas arenae
FIG|1548123.6.peg.1144; Pusillimonas sp. T2
FIG|380394.4.peg.276; Acidithiobacillus ferrooxidans ATCC 53993
WP_080292858.1;
FIG|101571.162.peg.3605; Burkholderia ubonensis
FIG|1382803.3.peg.22; Chromobacterium amazonense
FIG|930.4.peg.3851; Acidithiobacillus thiooxidans
FIG|1261658.3.peg.1787; Bibersteinia trehalosi Y31
FIG|1679001.3.peg.631; Pasteurellaceae bacterium NI1060
FIG|1334187.3.peg.653; Haemophilus influenzae KR494
FIG|1581107.3.peg.1286; Neisseria sp. HMSC15G01
FIG|486.24.peg.152; Neisseria lactamica
FIG|1953412.3.peg.1956; bacterium UBP10_UBA1160
WP_090322045.1; Nitrosomonas oligotropha
FIG|2013740.3.peg.1400; Deltaproteobacteria bacterium HGW-Deltaproteobacteria-13
FIG|1907413.3.peg.3170; Rhizobium sp. RU33A
FIG|1817963.3.peg.856; Roseomonas deserti
FIG|2035448.3.peg.1752; Rhizobium sp. C5
WP_014077019.1;
FIG|1648404.4.peg.2797; Erythrobacter atlanticus
FIG|359.11.peg.6331; Agrobacterium rhizogenes
FIG|887144.4.peg.573; Rhizobium taibaishanense
FIG|1116389.3.peg.333; Devosia insulae DS-56
FIG|121719.10.peg.3421; Pannonibacter phragmitetus
FIG|34002.6.peg.3570; Paracoccus alcaliphilus
FIG|1940281.4.peg.1560; Hoeflea sp.
FIG|1040981.5.peg.1561; Mesorhizobium ciceri WSM4083
FIG|410764.3.peg.807; Rhizobium multihospitium
FIG|1825934.3.peg.3111; Rhizobium anhuiense
FIG|1952824.3.peg.3061; Rhodobiaceae bacterium UBA3976
FIG|1871086.3.peg.2153; Brevundimonas sp.
FIG|588932.9.peg.647; Brevundimonas naejangsanensis
FIG|1951751.3.peg.1538; Erythrobacteraceae bacterium UBA1460
FIG|1843368.3.peg.904; Sphingobium sp. RAC03
FIG|155892.10.peg.3219; Caulobacter vibrioides
FIG|43057.4.peg.4537; Rhodobacter azotoformans
FIG|1514904.3.peg.974; Ahrensia marina
FIG|1338034.3.peg.722; Vibrio parahaemolyticus O1:Kuk str. FDA_R31
FIG|150340.18.peg.1837; Vibrio antiquarius
FIG|196024.5.peg.3821; Aeromonas dhakensis
FIG|244366.32.peg.1886; Klebsiella variicola
FIG|180957.35.peg.1654; Pectobacterium brasiliense
FIG|55601.149.peg.665; Vibrio anguillarum
FIG|121723.5.peg.2901; Photobacterium sp. SKA34
FIG|584.170.peg.837; Proteus mirabilis
FIG|40324.136.peg.3276; Stenotrophomonas maltophilia
FIG|1122188.5.peg.411; Lysobacter spongiicola DSM 21749
FIG|2032566.3.peg.2826; Xanthomonadaceae bacterium NML93-0792
FIG|287.1731.peg.2578; Pseudomonas aeruginosa
FIG|251702.3.peg.1529; Pseudomonas syringae pv. antirrhini
FIG|1960829.3.peg.5912; Pseudomonas sp. MF6394
FIG|76759.17.peg.5093; Pseudomonas monteilii
FIG|1981678.3.peg.5241; Pseudomonas sp. R45(2017)
FIG|1699620.3.peg.3028; Pseudomonas sp. RIT-PI-r
FIG|191391.4.peg.2140; Pseudomonas salomonii
FIG|1844093.4.peg.7190; Pseudomonas sp. 22 E 5
FIG|287.1744.peg.1414; Pseudomonas aeruginosa
FIG|287.1987.peg.910; Pseudomonas aeruginosa
FIG|287.4372.peg.4481; Pseudomonas aeruginosa
FIG|1856685.4.peg.2159; Pseudomonas sp. TCU-HL1
FIG|1718920.3.peg.3357; Pseudomonas sp. ICMP 8385
FIG|1781066.3.peg.2816; Duganella sp. HH101
FIG|95485.5.peg.60; Burkholderia stabilis
FIG|1572871.6.peg.588; Janthinobacterium sp. BJB304
WP_034208069.1; Burkholderia cepacia
WP_074283015.1; Burkholderia sp. GAS332
FIG|1168169.3.peg.2570; Methylomonas sp. 11b
FIG|1899355.16.peg.1328; Oceanospirillaceae bacterium
WP_093197597.1; Variovorax sp. YR750
FIG|1660091.3.peg.1650; Bordetella sp. SCN 67-23
FIG|134375.17.peg.4387; Achromobacter sp.
FIG|426114.10.peg.1990; Thiomonas arsenitoxydans
FIG|1947551.3.peg.1903; Stenotrophomonas sp. UBA2302
FIG|1914330.4.peg.2242; Salinisphaera sp.
FIG|1947037.3.peg.890; Pantoea sp. UBA5707
WP_094422719.1; Kosakonia cowanii
WP_079496884.1;
WP_088126255.1; Enterobacter kobei
WP_049614309.1; Yersinia
WP_048263135.1; Pectobacterium peruviense
WP_040197602.1; Klebsiella pneumoniae
FIG|669.34.peg.1586; Vibrio harveyi
FIG|672.219.peg.1032; Vibrio vulnificus
FIG|670.1028.peg.1775; Vibrio parahaemolyticus
WP_065207673.1; Photobacterium phosphoreum
FIG|1869214.3.peg.2231; Rheinheimera sp.
WP_029795910.1; Vibrio parahaemolyticus
FIG|1191302.3.peg.1081; Vibrio crassostreae 9ZC77
FIG|668.70.peg.1192; Aliivibrio fischeri
FIG|28229.4.peg.4229; Colwellia psychrerythraea
FIG|1855726.3.peg.270; Burkholderia sp. KK1
FIG|1674888.3.peg.829; Burkholderiales bacterium Beta_02
FIG|687412.4.peg.1108; Pseudorhodobacter aquimaris
WP_092465129.1; Donghicola eburneus
FIG|1120653.3.peg.5479; Ensifer sp. LC384
FIG|121719.5.peg.401; Pannonibacter phragmitetus
FIG|1798804.3.peg.1597; Rhizobium sp. 58
FIG|1946675.3.peg.3089; Kordiimonas sp. UBA4487
FIG|36861.5.peg.1400; Thiobacillus denitrificans
FIG|1115835.3.peg.1003; Methylotenera versatilis 79
FIG|1797188.3.peg.1508; Acidobacteria bacterium RIFCSPLOWO2_12_FULL_60_22
FIG|57320.3.peg.123; Pseudodesulfovibrio profundus
FIG|1267534.3.peg.1238; Acidobacteriaceae bacterium KBS 89
FIG|1951344.3.peg.527; Acidobacteriaceae bacterium UBA1307
WP_006226461.1; Achromobacter marplatensis
FIG|1503054.4.peg.5764; Burkholderia stagnalis
WP_006159686.1; Cupriavidus basilensis
WP_090191767.1; unclassified Duganella
FIG|539.8.peg.1698; Eikenella corrodens
FIG|1946925.3.peg.2129; Micavibrio sp. UBA5701
WP_047031309.1; Hoeflea sp. IMCC20628
FIG|1946134.3.peg.1092; Brevundimonas sp. UBA6547
WP_093914930.1; Sulfitobacter marinus
FIG|1862950.3.peg.1234; Rhizobiales bacterium NRL2
FIG|1166078.4.peg.1483; Aureimonas phyllosphaerae
FIG|709015.3.peg.734; Pontibacter actiniarum DSM 19842
WP_092160028.1; Desulfovibrio ferrireducens
FIG|2026749.3.peg.3364; Ignavibacteriae bacterium
WP_033771991.1; Pantoea agglomerans
WP_097097099.1; unclassified Enterobacteriaceae (miscellaneous)
FIG|1444151.3.peg.2733; Escherichia coli 2-177-06_S3_C2
WP_137545672.1; Escherichia coli Retron-Eco3 (Ec73)
FIG|573.14856.peg.3852; Klebsiella pneumoniae
WP_072021595.1; Serratia marcescens
FIG|29571.3.peg.478; Halomonas subglaciescola
WP_004534676.1;
WP_095622523.1; Halomonas sp. WRN001
FIG|376427.4.peg.3223; Halomonas gudaonensis
FIG|862908.3.peg.745; Halobacteriovorax marinus SJ
SCJ40239.1; uncultured Clostridium sp.
FIG|717962.3.peg.287; Coprococcus catus GD/7
WP_014642259.1; Halobacillus halophilus
FIG|2009042.3.peg.2106; Pseudomonas sp. Irchel 3H7
FIG|1981718.3.peg.4346; Pseudomonas sp. B39(2017)
FIG|665135.13.peg.1401; Pseudomonas sp. In5
FIG|1949067.3.peg.5629; Pseudomonas sp. PICF141
WP_007948552.1; Pseudomonas sp. GM21
SFB61662.1; Delftia tsuruhatensis
WP_011615687.1;
WP_014778098.1;
FIG|1429083.4.peg.2612; Pseudomonas hussainii
WP_095024014.1; Pseudomonas
WP_090203690.1; Pseudomonas asplenii
FIG|564423.8.peg.1646; Pseudomonas tolaasii NCPPB 2192
WP_078802277.1; Pseudomonas fluorescens
WP_090453229.1; Pseudomonas
FIG|1306420.5.peg.1032; Burkholderia pseudomallei MSHR5848
FIG|1357270.3.peg.1923; Pseudomonas syringae UB246
FIG|2018067.3.peg.2950; Pseudomonas sp. FDAARGOS_380
FIG|317.311.peg.3241; Pseudomonas syringae
FIG|287.2309.peg.126; Pseudomonas aeruginosa
WP_039522442.1; Pectobacterium brasiliense
WP_080861357.1; Klebsiella pneumoniae
WP_014542745.1; Erwinia sp. Ejp617
OSL25696.1; Escherichia coli TA255
FIG|1125693.3.peg.761; Proteus mirabilis WGLW4
KMK80587.1; Pectobacterium atrosepticum ICMP 1526
FIG|550.717.peg.2037; Enterobacter cloacae
ACS86154.1; Dickeya paradisiaca Ech703
WP_050122514.1; Yersinia frederiksenii
WP_081334048.1; Alteromonas macleodii
WP_055016254.1; Pseudoalteromonas sp. P1-13-1a
FIG|56799.5.peg.478; Colwellia sp.
FIG|666.3375.peg.2486; Vibrio cholerae
PIW62005.1; Shewanella sp. CG12_big_fil_rev_8_21_14_0_65_47_15
OCA54994.1; Photorhabdus namnaonensis
CNK75559.1; Yersinia frederiksenii
WP_024248662.1; Escherichia
WP_088618141.1; Methylovulum psychrotolerans
WP_051669880.1;
PCJ98666.1; Alteromonadaceae bacterium
WP_081919471.1; Acidithiobacillus ferrivorans
WP_055769167.1; Stenotrophomonas
WP_039422954.1; Xanthomonas vesicatoria
WP_078568253.1; Xanthomonas campestris
WP_093486747.1; unclassified Pseudoxanthomonas
WP_077445058.1; Rhodanobacter sp. C05
WP_092576562.1; Achromobacter sp. NFACC18-2
FIG|1330528.3.peg.2198; Escherichia coli NCCP 15656
FIG|83655.67.peg.2965; Leclercia adecarboxylata
FIG|573.10044.peg.2850; Klebsiella pneumoniae
WP_071888955.1; Enterobacterales
FIG|1799789.3.peg.4357; Paraglaciecola hydrolytica
FIG|2024839.8.peg.1563; Marinovum sp.
FIG|1381081.7.peg.1167; Vibrio panuliri
FIG|670.908.peg.3444; Vibrio parahaemolyticus
FIG|626887.3.peg.2431; Marinobacter nanhaiticus D15-8W
FIG|1913989.101.peg.1616; Gammaproteobacteria bacterium
FIG|262489.9.peg.2938; delta proteobacterium MLMS-1
FIG|2035207.3.peg.545; Janthinobacterium sp. 67
FIG|28095.13.peg.1040; Burkholderia gladioli
FIG|941449.3.peg.1262; Desulfovibrio sp. X2
FIG|1768806.3.peg.778; Rhodospirillaceae bacterium CCH5-H10
WP_083634830.1; Desulfovibrio sp. DV
FIG|1231.4.peg.574; Nitrosospira multiformis
FIG|604089.3.peg.1142; Flavobacterium sinopsychrotolerans
FIG|357523.3.peg.1851; Flavobacterium sp. 11
FIG|1423323.5.peg.321; Flavobacterium sp. AED
FIG|178356.3.peg.502; Flavobacterium xinjiangense
FIG|1946545.3.peg.3457; Flavobacterium sp. UBA4120
FIG|150146.3.peg.2822; Flavobacterium gillisiae
FIG|229203.4.peg.1981; Flavobacterium degerlachei
FIG|280093.5.peg.432; Flavobacterium granuli
FIG|728056.4.peg.1154; Flavobacterium oncorhynchi
FIG|143224.8.peg.2343; Zobellia uliginosa
FIG|1225176.3.peg.4300; Cecembia lonarensis LW9
FIG|1434700.3.peg.581; Moheibacter sediminis
FIG|996.47.peg.468; Flavobacterium columnare
FIG|172045.56.peg.2231; Elizabethkingia miricola
FIG|2024823.3.peg.2086; Altibacter sp.
FIG|2026728.18.peg.4090; Crocinitomicaceae bacterium
FIG|980584.3.peg.2930; Aquimarina agarivorans
FIG|1946744.3.peg.1682; Leeuwenhoekiella sp. UBA1003
FIG|1046627.3.peg.2526; Bizionia argentinensis JUB59
FIG|906888.15.peg.37; Nonlabens ulvanivorans
FIG|407022.4.peg.2865; Olivibacter domesticus
FIG|1500282.3.peg.3713; Chryseobacterium sp. CF365
WP_084550290.1; Chryseobacterium scophthalmum
FIG|190304.8.peg.741; Fusobacterium nucleatum subsp. nucleatum ATCC 25586
FIG|1352.1731.peg.603; Enterococcus faecium
FIG|1428.658.peg.666; Bacillus thuringiensis
FIG|1497681.3.peg.3095; Listeria newyorkensis
FIG|1396.1440.peg.4237; Bacillus cereus
FIG|1917876.3.peg.2997; Blautia sp. Marseille-P3087
FIG|1952168.3.peg.215; Lachnospiraceae bacterium UBA7480
FIG|1907659.3.peg.1085; Blautia sp. Marseille-P3201T
FIG|1265309.16.peg.461; Epibacterium mobile F1926
FIG|853.163.peg.215; Faecalibacterium prausnitzii
FIG|1264.5.peg.4; Ruminococcus albus
FIG|1500289.3.peg.4469; Chryseobacterium sp. OV705
FIG|1197728.3.peg.2386; Prevotella conceptionensis 9403948
FIG|1947486.3.peg.2515; Sphingobacterium sp. UBA1897
FIG|529.12.peg.1303; Ochrobactrum anthropi
FIG|1523429.3.peg.2936; Rhizobium sp. AAP116
FIG|1761878.3.peg.469; Paenibacillus sp. cl6col
FIG|1462996.4.peg.2634; Paenibacillus yonginensis
FIG|582475.4.peg.4724; Lysinibacillus xylanilyticus
FIG|1773.7915.peg.7638; Mycobacterium tuberculosis
FIG|360310.3.peg.4853; Bacillus sp. CDB3
FIG|1396.515.peg.2936; Bacillus cereus
FIG|662367.4.peg.242; Spirosoma endophyticum
FIG|1895719.3.peg.2950; Bacteroidales bacterium 45-6
FIG|906888.9.peg.926; Nonlabens ulvanivorans
FIG|694433.3.peg.2346; Saprospira grandis DSM 2844
FIG|1167006.5.peg.2941; Desulfocapsa sulfexigens DSM 10523
FIG|649724.3.peg.304; Clostridium sp. ATCC BAA-442
FIG|1505.32.peg.2959; Paeniclostridium sordellii
FIG|1953142.3.peg.1858; Bacteroidetes bacterium UBA1947
FIG|2029590.3.peg.2754; Mucilaginibacter sp. MD40
FIG|29581.33.peg.2300; Janthinobacterium lividum
FIG|40324.292.peg.236; Stenotrophomonas maltophilia
FIG|1403329.3.peg.287; Listeria monocytogenes Lm25180
FIG|1121865.3.peg.1262; Enterococcus columbae DSM 7374 = ATCC 51263
FIG|1120746.3.peg.3113; bacterium MS4
FIG|1952299.3.peg.221; Ruminococcaceae bacterium UBA2656
FIG|1965604.3.peg.686; Anaeromassilibacillus sp. An250
FIG|1673717.3.peg.805; Anaeromassilibacillus senegalensis
WP_116884683.1; Victivallis vadensis
FIG|1948697.3.peg.196; Lentisphaeria bacterium UBA4640
FIG|1232460.3.peg.46; Clostridiales bacterium VE202-28
WP_007864340.1; Clostridiales
WP_055649738.1; Hungatella hathewayi
FIG|1226325.3.peg.2005; Clostridium sp. KLE 1755
FIG|1432052.10.peg.3 166; Eisenbergiella tayi
FIG|208479.8.peg.4376; Enterocloster bolteae
FIG|1298920.3.peg.1959; [Desulfotomaculum] guttoideum DSM 4024
FIG|1776047.3.peg.4241; Clostridium sp. C105KSO15
FIG|1946596.3.peg.2399; Hungatella sp. UBA4396
FIG|1946603.3.peg.924; Hungatella sp. UBA7603
FIG|1410651.3.peg.407; [Clostridium] aerotolerans DSM 5434
FIG|1697784.3.peg.9617; Clostridia bacterium UC5.1-1D4
FIG|1745713.3.peg.3865; Bariatricus massiliensis
FIG|180332.3.peg.1515; Robinsoniella peoriensis
WP_072851604.1; Lactonifactor longoviformis
WP_003507561.1; Clostridiales
FIG|1111728.3.peg.587; Budvicia aquatica DSM 5075 = ATCC 35567
FIG|1122977.4.peg.2473; Pragia fontium DSM 5563 = ATCC 49100
FIG|1950915.3.peg.189; Clostridiales bacterium UBA644
FIG|1950927.3.peg.912; Clostridiales bacterium UBA7187
ERK60856.1; Oscillibacter sp. KLE 1728
WP_009260579.1; Flavonifractor plautii
FIG|1235797.3.peg.2409; Oscillibacter sp. 1-3
FIG|1520815.3.peg.1262; Ruminococcaceae bacterium D5
FIG|1855302.3.peg.1138; Pseudobutyrivibrio sp. JW11
FIG|43305.5.peg.3631; Butyrivibrio proteoclasticus
FIG|411463.15.peg.1791; Eubacterium ventriosum ATCC 27560
FIG|1235792.3.peg.3837; Lachnospiraceae bacterium M18-1
FIG|97139.3.peg.669; Schaedlerella arabinosiphila
FIG|1291051.3.peg.1165; Mediterraneibacter glycyrrhizinilyticus JCM 13369
FIG|1532.6.peg.4793; Blautia coccoides
FIG|1121114.4.peg.5478; Blautia producta ATCC 27340 = DSM 2950
FIG|1262776.3.peg.1908; Clostridium sp. CAG:149
FIG|1262792.3.peg.1164; Clostridium sp. CAG:299
FIG|1262995.3.peg.2852; Firmicutes bacterium CAG:646
FIG|537007.17.peg.3146; Blautia hansenii DSM 20583
FIG|1965569.3.peg.1928; Lachnoclostridium sp. An169
FIG|1952411.3.peg.2018; Ruminococcaceae bacterium UBA6353
FIG|1965578.3.peg.1947; Pseudoflavonifractor sp. An187
WP_001775049.1; Escherichia coli Retron-Eco5 (Ec107)
WP_012602583.1;
WP_015962464.1; Enterobacteriaceae bacterium strain FGI 57
FIG|1005999.3.peg.3342; Leminorella grimontii ATCC 33999 = DSM 5078
FIG|1378073.3.peg.795; Enterobacter sp. CC120223-11
FIG|911023.3.peg.138; Yokenella regensburgei ATCC 49455
FIG|1834193.3.peg.4113; Enterococcus sp. 9E7_DIV0242
FIG|1649188.10.peg.406; Listeria goaensis
FIG|1430899.3.peg.278; Listeria fleischmannii 1991
FIG|1211844.4.peg.748; Candidatus Stoquefichus massiliensis AP9
FIG|1658109.3.peg.34; Candidatus Stoquefichus sp. SB1
FIG|1262793.3.peg.950; Clostridium sp. CAG:302
FIG|1262908.3.peg.1120; Mycoplasma sp. CAG:956
FIG|1674844.3.peg.242; Clostridiales bacterium Firm_06
FIG|1410672.3.peg.2823; Ruminococcus flavefaciens ND2009
FIG|1947424.3.peg.1718; Ruminococcus sp. UBA4310
FIG|1265.9.peg.2602; Ruminococcus flavefaciens
FIG|1336236.3.peg.1817; Ruminococcus flavefaciens ATCC 19208
CDC65895.1; Ruminococcus sp. CAG:57
WP_092946213.1; Ruminococcaceae bacterium YRB3002
FIG|1307.1644.peg.1532; Streptococcus suis
WP_050516365.1; Escherichia coli
WP_097505494.1; Escherichia coli Retron-Eco5 (Ec107)
FIG|573.15585.peg.2343; Klebsiella pneumoniae
WP_023581669.1; Proteus hauseri
WP_079656969.1; Serratia marcescens
WP_090085157.1; Phytobacter sp. SCO41
FIG|573.15584.peg.1543; Klebsiella pneumoniae
WP_023330997.1; Enterobacter cloacae complex
FIG|72407.673.peg.2552; Klebsiella pneumoniae subsp. pneumoniae
CNM01182.1; Yersinia pseudotuberculosis
CNG88012.1; Yersinia enterocolitica
FIG|1925763.3.peg.649; Marinobacter salexigens
PKW24121.1; Marinobacter sp. LV10R510-8
WP_045597342.1; Vibrio vulnificus
WP_098972386.1; Aeromonas sp. CU5
WP_005172873.1; Yersinia enterocolitica
WP_052979504.1; Enterobacteriaceae
WP_083069261.1; Pantoea vagans
WP_053911905.1; Pseudoalteromonas sp. SW0106-04
FIG|1916082.18.peg.39; Alteromonadaceae bacterium
WP_046555216.1; Arsukibacterium sp. MJ3
KPW01986.1; Pseudoalteromonas sp. P1-8
WP_094277737.1; Oceanimonas baumannii
FIG|1414654.3.peg.2005; Oceanisphaera psychrotolerans
WP_008133621.1; unclassified Pseudoalteromonas
KQA22543.1; Vibrio metoecus
WP_000284440.1; Vibrio cholerae
WP_011261677.1; Aliivibrio fischeri
KEE40622.1;
WP_012982829.1;
ALL66139.1; Paraburkholderia caribensis MBA4
WP_093223969.1; Pseudomonas vancouverensis
FIG|2015553.3.peg.2940; Pseudomonas sp. PGPPP1
WP_096082869.1; Pseudomonas aeruginosa
ONM67687.1; Pseudomonas aeruginosa
FIG|316.213.peg.2906; Pseudomonas stutzeri
WP_078734267.1; Pseudomonas fluorescens
WP_079384669.1; Pseudomonas aeruginosa
WP_095948157.1; Variovorax boronicumulans
WP_011625020.1; Shewanella sp. MR-7
WP_100292553.1; Aeromonas cavernicola
WP_055021484.1; Pseudoalteromonas sp. P1-26
PHS01491.1; Oceanobacter sp.
FIG|2024618.3.peg.1141; Acinetobacter sp. BS1
WP_114139108.1; Klebsiella pneumoniae
WP_077749737.1; Pseudomonas sp. FSL W5-0299
WP_078451378.1; Pseudomonas aeruginosa
WP_007245785.1; Pseudomonas syringae group
FIG|316.280.peg.1454; Pseudomonas stutzeri
WP_086822222.1; Pseudomonas aeruginosa
WP_073268605.1; Pseudomonas punonensis
WP_095280108.1; Lelliottia jeotgali
WP_095715328.1; Citrobacter sp. TSA-1
WP_050111525.1; Yersinia
WP_013724211.1; Aeromonas veronii
WP_021140819.1; Aeromonas salmonicida
FIG|1094342.5.peg.1611; Alcanivorax xenomutans
FIG|1932666.4.peg.1886; Haliea sp.
WP_087148323.1; Crenothrix polyspora
WP_064022638.1; Methylomonas sp. DH-1
PIY64876.1; Shewanella sp. CG_4_10_14_0_8 um_filter_42_13
WP_006710190.1; Vibrio ichthyoenteri
WP_045040928.1; Photobacterium iliopiscarium
WP_054543201.1; Vibrio splendidus
WP_080540293.1; Vibrio vulnificus
FIG|2032624.3.peg.2540; Halomonas sp. WN018
KJT50308.1; Salmonella enterica subsp. enterica serovar Heidelberg str. RI-11-014588 Retron-Sen1 (Se72)
WP_005761319.1;
ODQ05744.1; Shigella sp. FC130
KKW01006.1; Candidatus Saccharibacteria bacterium GW2011_GWC2_48_9
KMZ12260.1; Candidatus Burkholderia humilis
SFQ04394.1; Ralstonia sp. NFACC01
WP_025373922.1; Advenella mimigardefordensis
WP_093341200.1; Variovorax sp. PDC80
WP_091453700.1; Giesbergeria anulus
SAY51889.1; Neisseria weaveri
WP_065255232.1; Moraxella lacunata
WP_049330876.1; Neisseria
FIG|1196095.197.peg.151; Gilliamella apicola
WP_072956843.1; Vibrio gazogenes
FIG|857087.3.peg.3286; Methylomonas methanica MC09
FIG|1952222.3.peg.1307; Methylococcaceae bacterium UBA3127
WP_039486261.1; Vibrio sinaloensis
WP_065545234.1; Vibrio scophthalmi
WP_033094845.1; Colwellia psychrerythraea
WP_057552475.1; Vibrio cholerae
WP_004726393.1; Vibrio furnissii
FIG|2020862.3.peg.1934; Halobacteriovorax sp.
FIG|624.1260.peg.1437; Shigella sonnei
WP_011516221.1; Burkholderiales
FIG|1947370.3.peg.1923; Pusillimonas sp. UBA4517
WP_038400955.1; Yersinia pseudotuberculosis
FIG|1951903.3.peg.117; Halieaceae bacterium UBA3099
WP_024914507.1; Chania multitudinisentens
WP_042893228.1; Enterobacteriaceae
WP_038238211.1; Xenorhabdus szentirmaii
EXI65661.1; Candidatus Accumulibacter sp. SK-12
WP_016452106.1; Delftia
WP_013517170.1; Alicycliphilus denitrificans
OXC73828.1; Caballeronia sordidicola
AIO65205.1; Burkholderia oklahomensis
WP_013234866.1; Herbaspirillum seropedicae
WP_082884385.1; Piscirickettsiaceae bacterium NZ-RLO1
FIG|2006849.4.peg.371; Xanthomonadales bacterium
WP_074262787.1; Paraburkholderia phenazinium
WP_009906786.1; Burkholderia thailandensis
WP_022524328.1;
WP_081817450.1; Halomonas sp. HL-48
WP_020312233.1; Pseudomonas syringae
KPY75916.1; Pseudomonas amygdali pv. tabaci
FIG|1793966.3.peg.180; Pseudomonas fluvialis
FIG|1891229.16.peg.2033; Pseudomonadales bacterium
WP_099454886.1; Pseudomonas putida
WP_092400423.1; Pseudomonas sp. NFACC39-1
WP_012315430.1; Pseudomonas putida
WP_020799819.1; Pseudomonas sp. G5(2012)
WP_004574016.1;
FIG|1435425.3.peg.787; Pseudomonas sp. QTF5
WP_045490543.1; Pseudomonas sp. StFLB209
WP_011506503.1; Chromohalobacter salexigens
FIG|1609967.3.peg.3047; Halomonas sp. HG01
FIG|1492738.3.peg.2698; Flavobacterium seoulense
WP_092849245.1; Algibacter pectinivorans
WP_025835957.1; Bacteroides
FIG|2025877.3.peg.668; Parabacteroides sp. AT13
FIG|246787.6.peg.2081; Bacteroides cellulosilyticus
FIG|1339287.3.peg.1113; Bacteroides fragilis str. 3986 T(B)9
FIG|1946017.3.peg.1516; Alistipes sp. UBA940
WP_038655380.1; Mucinivorans hirudinis
WP_093669272.1; Tenacibaculum sp. MAR_2009_124
WP_073241067.1; Flavobacterium flevense
WP_096193803.1; Cytophagales bacterium TFI 002
WP_076357635.1;
WP_073238193.1; Pedobacter caeni
WP_076451370.1;
WP_091906542.1; Porphyromonadaceae bacterium KH3R12
WP_051365712.1; Flavobacterium saliperosum
FIG|1938609.3.peg.1765; Flavobacterium sp. LM4
SDJ72221.1; Flavobacterium noncentrifugens
FIG|1985174.3.peg.2584; Chitinophagaceae bacterium IBVUCB2
WP_092737749.1; Riemerella columbipharyngis
FIG|192149.3.peg.42; Muricauda sp.
FIG|418630.3.peg.1685; Rhodobacter megalophilus
FIG|1915314.3.peg.3469; Thioclava sp. DLFJ5-1
FIG|2030815.3.peg.2725; Marinosulfonomonas sp.
FIG|2035451.3.peg.4632; Rhizobium sp. L18
WP_043872258.1; Celeribacter indicus
WP_055683826.1; Jannaschia rubra
FIG|1947537.3.peg.498; Sphingopyxis sp. UBA6198
WP_069065961.1; Sphingobium sp. RAC03
WP_084280100.1; Novosphingobium sp. B1
FIG|1895845.3.peg.487; Sphingobium sp. 66-54
GAK73419.1; Agrobacterium rubi TR3 = NBRC 13261
WP_090966398.1; Aureimonas phyllosphaerae
WP_091860144.1; Bosea robiniae
WP_085092006.1; Azospirillum oryzae
FIG|1528100.4.peg.28; Methylomagnum ishizawai
FIG|32057.3.peg.9515; Calothrix sp. PCC 7103
FIG|103690.10.peg.3571; Nostoc sp. PCC 7120 = FACHB-418
FIG|1137095.11.peg.15; Scytonema sp. HK-05
CDZ48826.1; Neorhizobium galegae bv. officinalis
WP_072340070.1; Devosia enhydra
OYR18277.1; Ochrobactrum thiophenivorans
WP_093509439.1; Sphingopyxis sp. YR583
WP_081799025.1; Novosphingobium resinovorum
PIY55545.1; Zetaproteobacteria bacterium CG_4_10_14_0_8_um_filter_49_80
SDT44912.1; Bradyrhizobium canariense
WP_096350346.1;
WP_074962594.1; Jannaschia rubra
WP_038724888.1; Burkholderia pseudomallei
WP_012217410.1; Burkholderia multivorans
WP_100428762.1; Janthinobacterium sp. 67
WP_082161008.1; Candidatus Competibacter denitrificans
AFL73219.1; Thiocystis violascens DSM 198
WP_014427842.1;
FIG|364030.3.peg.3554; Thiomonas delicata
KGW20495.1; Burkholderia pseudomallei MSHR2451
SFE83076.1; Variovorax sp. OK212
WP_013028226.1; Sideroxydans lithotrophicus
WP_080311424.1; Burkholderia pseudomallei
FIG|337.13.peg.3872; Burkholderia glumae
WP_082643860.1; Pseudomonas
CKH90039.1; Pseudomonas aeruginosa
WP_083287254.1; unclassified Janthinobacterium
WP_122648546.1; Burkholderia pseudomallei
WP_082706753.1; unclassified Pseudomonas
WP_080936076.1; Klebsiella pneumoniae
WP_000746343.1; Enterobacteriaceae
EMX54653.1; Escherichia coli MP020980.2
WP_053270700.1; Escherichia coli
FIG|1736224.3.peg.3731; Serratia sp. Leaf51
FIG|1175299.4.peg.709; Dickeya zeae ZJU1202
WP_001461245.1; Enterobacteriaceae
FIG|617145.3.peg.3535; Vibrio splendidus 1F-157
FIG|1440054.3.peg.3851; Vibrio sp. OY15
FIG|617135.3.peg.594; Aliivibrio fischeri ZF-211
WP_023267764.1; Shewanella decolorationis
FIG|1481663.36.peg.3628; Vibrio metoecus
FIG|670.893.peg.2716; Vibrio parahaemolyticus
FIG|680.33.peg.5391; Vibrio campbellii
FIG|298386.8.peg.4344; Photobacterium profundum SS9
FIG|663.73.peg.714; Vibrio alginolyticus
FIG|1333511.3.peg.3208; Pseudoalteromonas haloplanktis TAB23
WP_064574154.1; Hafnia paralvei
WP_064645509.1; Obesumbacterium proteus
FIG|630.105.peg.4248; Yersinia enterocolitica
FIG|400673.7.peg.1969; Legionella pneumophila str. Corby
WP_092678546.1; Rosenbergiella nectarea
WP_069476513.1; Raoultella ornithinolytica
FIG|1267535.3.peg.2394; Bryobacterales bacterium KBS 96
WP_000446053.1; Acinetobacter baumannii
FIG|1948587.3.peg.786; Gammaproteobacteria bacterium UBA1902
WP_014949305.1; Alteromonas macleodii
FIG|1797397.3.peg.2386; Bdellovibrionales bacterium RIFOXYC1_FULL_54_43
FIG|1386968.3.peg.847; Francisella tularensis subsp. novicida PA10-7858
WP_074900850.1;
FIG|1975705.3.peg.898; Psychrobacter sp. FDAARGOS_221
WP_066184577.1; Arcobacter
FIG|1780380.4.peg.4010; Eubacteriaceae bacterium CHKCI004
FIG|556261.3.peg.2546; Clostridium sp. D5
FIG|1193534.6.peg.2375; uncultured Flavonifractor sp.
FIG|1042163.3.peg.3771; Brevibacillus laterosporus LMG 15441
WP_062492190.1; Paenibacillus sp. 32O-W
WP_081674606.1; Lactobacillus harbinensis
WP_050781686.1; Lactobacillus coryniformis
WP_021109137.1; Enterococcus faecium
WP_046309803.1; Staphylococcus
CBL03706.1; Gordonibacter pamelaeae 7-10-1-b
WP_090944285.1; Pelosinus propionicus
WP_077305443.1; Clostridium beijerinckii
FIG|410072.5.peg.40; Coprococcus comes
WP_011669870.1; Leptospira borgpetersenii
WP_015565235.1; Faecalibacterium prausnitzii
CUO23478.1; Faecalibacterium prausnitzii
WP_085748688.1; Rhizobacter gummiphilus
WP_093270014.1; Psychrobacillus sp. OK032
SHE86352.1; Atopostipes suicloacalis DSM 15692
WP_000346292.1; unclassified Streptococcus
WP_080465410.1; Lactobacillus plantarum
WP_080662531.1; Lactobacillus brevis
WP_093131554.1; Salinibacillus kushneri
WP_093336905.1; Salibacterium halotolerans
FIG|1974627.3.peg.386; Candidatus Levybacteria bacterium CG_4_9_14_0_2_um_filter_35_21
FIG|1802603.3.peg.453; Candidatus Woykebacteria bacterium
RIFCSPHIGHO2_12_FULL_45_10
FIG|392734.5.peg.3006; Terriglobus roseus
AGL61879.1; Candidatus Saccharimonas aalborgensis
FIG|319224.16.peg.2726; Shewanella putrefaciens CN-32
FIG|1720343.3.peg.1263; Pseudoalteromonas sp. 1_2015MBL_MicDiv
FIG|1136158.3.peg.3691; Vibrio cyclitrophicus 1F97
FIG|666.3017.peg.1000; Vibrio cholerae
FIG|1909458.3.peg.2277; Salinivibrio sp. ML198
FIG|1638949.3.peg.831; Vibrio sp. ECSMB14106
FIG|493915.3.peg.158; Pseudoalteromonas sp. NJ631
BAC94535.1; Vibrio vulnificus YJ016
FIG|1191313.3.peg.1135; Vibrio splendidus 1S-124
FIG|670.1244.peg.3807; Vibrio parahaemolyticus
FIG|1659714.3.peg.4264; Citrobacter braakii
FIG|1192730.4.peg.1976; Salmonella enterica subsp. enterica serovar Kintambo
FIG|550.1216.peg.4296; Enterobacter cloacae
WP_072269713.1; Serratia
WP_053898075.1; Escherichia coli
FIG|624.1264.peg.1635; Shigella sonnei
FIG|1181777.3.peg.78; Escherichia coli KTE233
FIG|1802256.3.peg.310; Sulfurimonas sp. RIFOXYB12_FULL_35_9
PHR73342.1; Arcobacter sp.
FIG|2014260.3.peg.3813; bacterium (Candidatus Blackallbacteria)
CG13_big_fil rev_8_21_14_2_50_49_14
WP_042497590.1; Vibrio maritimus
WP_063522799.1; Vibrio sp. HI00D65
WP_004186757.1; Enterobacteriaceae
WP_040122746.1; Vibrio
WP_086046550.1; Vibrio harveyi group
WP_063849005.1; Enterobacter cloacae
WP_023486614.1; Enterobacteriaceae
WP_070992278.1; Pseudoalteromonas byunsanensis
FIG|1005665.3.peg.2532; Kosakonia oryzendophytica
FIG|1219066.3.peg.3636; Vibrio parahaemolyticus NBRC 12711
FIG|1225184.4.peg.1222; Pantoea sp. A4
FIG|675814.3.peg.1256; Vibrio coralliilyticus ATCC BAA-450
SFR59865.1; Pseudobutyrivibrio sp. NOR37
FIG|853.16.peg.1112; Faecalibacterium prausnitzii
FIG|1965572.3.peg.1423; Pseudoflavonifractor sp. An176
FIG|588581.3.peg.3589; Ruminiclostridium papyrosolvens DSM 2782
FIG|1396.1409.peg.4169; Bacillus cereus
FIG|1428.538.peg.4047; Bacillus thuringiensis
FIG|1465.16.peg.946; Brevibacillus laterosporus
WP_087385137.1;
AIF42417.1; Virgibacillus sp. SK37
WP_076543941.1; Halanaerobium kushneri
FIG|1121093.3.peg.3089; Bacillus panaciterrae DSM 19096
FIG|29367.3.peg.2029; Clostridium puniceum
WP_089719707.1; Halanaerobium congolense
FIG|307249.3.peg.3585; uncultured Sporomusa sp.
WP_072949666.1; Ruminococcus flavefaciens
CCX81854.1; Ruminococcus sp. CAG:108
FIG|1491.669.peg.2217; Clostridium botulinum
FIG|1872455.3.peg.401; Alkaliphilus sp.
FIG|576117.5.peg.4005; Celeribacter halophilus
FIG|1225647.3.peg.1829; Phaeobacter sp. 11ANDIMAR09
FIG|1380380.4.peg.1574; Ahrensia sp. 13_GOM-1096m
FIG|293.7.peg.2956; Brevundimonas diminuta
WP_095437634.1; Rhizobium sp. 11515TR
FIG|1912891.7.peg.702; Sphingobium sp.
FIG|1736574.3.peg.4024; Pseudoxanthomonas sp. Root630
FIG|227946.13.peg.4105; Xanthomonas translucens pv. poae
FIG|1761791.3.peg.4793; Lysobacter sp. yr284
FIG|1560195.5.peg.485; Janthinobacterium sp. BJB301
FIG|1503054.43.peg.6257; Burkholderia stagnalis
FIG|1207504.10.peg.4279; Burkholderia pseudomultivorans
WP_092172515.1; unclassified Pseudomonas
WP_074815429.1; Pseudomonas syringae
FIG|150146.3.peg.3162; Flavobacterium gillisiae
FIG|76832.8.peg.3775; Myroides odoratimimus
FIG|1202724.3.peg.994; Flavobacterium akiainvivens
FIG|1805473.3.peg.3678; Chryseobacterium timonianum
FIG|253.33.peg.3826; Chryseobacterium indologenes
WP_076561634.1; Chryseobacterium indoltheticum
FIG|2024823.3.peg.95; Altibacter sp.
FIG|1250278.4.peg.3462; Salegentibacter sp. Hel_I_6
FIG|1797342.3.peg.689; Bacteroidetes bacterium GWF2_33_38
WP_084184261.1; Chryseobacterium ureilyticum
FIG|1948560.3.peg.3003; Deltaproteobacteria bacterium UBA6106
FIG|1392.364.peg.2564; Bacillus anthracis
FIG|872970.3.peg.1713; Amphibacillus marinus
FIG|1385514.3.peg.313; Pontibacillus yanchengensis Y32
FIG|76853.4.peg.2614; Solibacillus silvestris
FIG|1423774.3.peg.1262; Lactobacillus nantensis DSM 16982
FIG|1410670.3.peg.2844; Ruminococcus flavefaciens MA2007
FIG|169435.7.peg.1348; Anaerotruncus colihominis
FIG|1946597.3.peg.2104; Hungatella sp. UBA4568
FIG|1948087.3.peg.796; Firmicutes bacterium UBA6113
FIG|642492.3.peg.2638; Cellulosilyticum lentocellum DSM 5427
FIG|1950841.3.peg.2383; Clostridiales bacterium UBA2436
FIG|555512.3.peg.1251; Salipiger marinus
FIG|383381.3.peg.2538; Erythrobacter sp. JL475
WP_081629462.1;
FIG|1736258.3.peg.3392; Methylobacterium sp. Leaf112
FIG|1950192.3.peg.426; Anaerolineales bacterium UBA2232
FIG|170623.6.peg.4661; Azotobacter beijerinckii
FIG|170623.7.peg.704; Azotobacter beijerinckii
FIG|1981099.3.peg.513; Niveispirillum lacus
FIG|1250539.3.peg.3491; Pelagibaca abyssi
FIG|1947582.3.peg.2979; Sulfitobacter sp. UBA1132
FIG|1909294.17.peg.3456; Rhizobiales bacterium
FIG|1735583.3.peg.1657; Pseudovibrio sp. W64
FIG|670.1220.peg.4688; Vibrio parahaemolyticus
FIG|1004786.3.peg.925; Alteromonas mediterranea DE1
FIG|2013797.3.peg.2109; Gammaproteobacteria bacterium HGW-Gammaproteobacteria-15
FIG|1948580.3.peg.3400; Gammaproteobacteria bacterium UBA1012
FIG|1714300.3.peg.306; Marinobacterium profundum
FIG|1961547.3.peg.1371; Desulfobulbaceae bacterium UBA2273
FIG|441162.10.peg.6621; Burkholderia oklahomensis C6786
FIG|615.307.peg.4666; Serratia marcescens
FIG|631.3.peg.1883; Yersinia intermedia
FIG|1763535.3.peg.1547; Hydrogenophaga crassostreae
FIG|43263.5.peg.2702; Pseudomonas alcaligenes
FIG|244366.46.peg.3595; Klebsiella variicola
FIG|1224150.8.peg.3856; Dickeya paradisiaca NCPPB 2511
FIG|61645.10.peg.2019; Enterobacter asburiae
FIG|1948706.3.peg.2225; Opitutae bacterium UBA1333
FIG|2026771.13.peg.1697; Opitutae bacterium
FIG|2026771.11.peg.1955; Opitutae bacterium
FIG|2026772.5.peg.424; Opitutales bacterium
FIG|2026801.20.peg.1798; Verrucomicrobiales bacterium
FIG|2026801.14.peg.1176; Verrucomicrobiales bacterium
FIG|1951369.3.peg.1157; Akkermansiaceae bacterium UBA6946
FIG|1977087.12.peg.1918; Proteobacteria bacterium
FIG|2026779.14.peg.4171; Planctomycetaceae bacterium
FIG|2026779.28.peg.3264; Planctomycetaceae bacterium
FIG|2026779.30.peg.3181; Planctomycetaceae bacterium
FIG|2026779.29.peg.2310; Planctomycetaceae bacterium
FIG|1797235.3.peg.3; Acinetobacter sp. RIFCSPHIGHO2_12_41_5
FIG|316.284.peg.937; Pseudomonas stutzeri
FIG|296.11.peg.442; Pseudomonas fragi
FIG|1981714.3.peg.993; Pseudomonas sp. B5(2017)
FIG|50340.44.peg.6020; Pseudomonas fuscovaginae
FIG|1761897.3.peg.509; Pseudomonas sp. ok272
FIG|1402514.3.peg.154; Pseudomonas aeruginosa BWHPSA014
FIG|1938440.3.peg.5997; Pseudomonas sp. T
FIG|1566250.3.peg.959; Pseudomonas sp. NFACC02
FIG|316.357.peg.479; Pseudomonas stutzeri
FIG|287.4433.peg.2945; Pseudomonas aeruginosa
FIG|1970515.3.peg.709; Hydrogenophilales bacterium 12-61-10
FIG|95486.85.peg.1748; Burkholderia cenocepacia
FIG|292.61.peg.8104; Burkholderia cepacia
FIG|1408450.3.peg.3766; Methylobacter tundripaludum 21/22
FIG|157910.3.peg.5727; Paraburkholderia tuberum
FIG|279058.16.peg.4239; Collimonas arenae
FIG|1537272.3.peg.1916; Janthinobacterium sp. HH100
FIG|1218081.3.peg.1751; Paraburkholderia kururiensis subsp. thiooxydans NBRC 107107
FIG|573.14059.peg.3113; Klebsiella pneumoniae
FIG|40324.192.peg.51; Stenotrophomonas maltophilia
FIG|1219041.3.peg.4613; Sphingomonas azotifigens NBRC 15497
FIG|1561196.3.peg.560; Burkholderia sp. E7m39
FIG|1882750.3.peg.1035; Burkholderia sp. GAS332
FIG|1736266.3.peg.1145; Duganella sp. Leaf126
FIG|2015350.3.peg.1640; Burkholderia sp. AU18528
FIG|58133.4.peg.815; Nitrosospira sp. NpAV
FIG|1691980.3.peg.1912; Rhodocyclaceae bacterium Paddy-1
FIG|305.393.peg.1023; Ralstonia solanacearum
FIG|56449.3.peg.3604; Xanthomonas bromi
FIG|1281282.5.peg.1894; Xanthomonas campestris pv. campestris str. CN14
FIG|40324.334.peg.1103; Stenotrophomonas maltophilia
FIG|1349793.3.peg.2529; Hydrogenophaga taeniospiralis NBRC 102512
FIG|1842727.3.peg.1491; Rhodoferax koreense
FIG|1619952.3.peg.5158; Burkholderiaceae bacterium 16
FIG|1970380.3.peg.1914; Halothiobacillus sp. 14-55-98
FIG|2015568.3.peg.2963; Burkholderiales bacterium PBB6
FIG|1752215.3.peg.23 12; Gammaproteobacteria bacterium Ga0077554
FIG|1706231.5.peg.3125; Janthinobacterium sp. CG23_2
FIG|2013716.3.peg.2169; Betaproteobacteria bacterium HGW-Betaproteobacteria-4
FIG|1946997.3.peg.3049; Nitrospira sp. UBA7655
FIG|765913.3.peg.2527; Thiorhodococcus drewsii AZ1
FIG|1743159.3.peg.1891; Polynucleobacter yangtzensis
FIG|1597955.3.peg.3923; Limnohabitans sp. DM1
FIG|1184267.3.peg.1626; Bdellovibrio exovorus JSS
FIG|101571.190.peg.3007; Burkholderia ubonensis
FIG|123899.5.peg.1710; Bordetella trematum
FIG|463035.3.peg.3900; Bordetella genomosp. 12
FIG|1395608.4.peg.211; Bordetella genomosp. 5
FIG|1947379.3.peg.2784; Rhodoferax sp. UBA5149
WP_074294985.1; Paraburkholderia phenazinium
FIG|1324617.3.peg.820; Paraburkholderia aspalathi
FIG|80868.3.peg.3458; Acidovorax cattleyae
FIG|1388764.3.peg.1840; Pseudogulbenkiania ferrooxidans EGD-HP2
FIG|251747.15.peg.4695; Chromobacterium subtsugae
FIG|670.1020.peg.382; Vibrio parahaemolyticus
FIG|1055803.3.peg.1434; Pseudoalteromonas sp. TB51
FIG|1201036.3.peg.177; Pseudochrobactrum sp. AO18b
FIG|1220581.4.peg.1434; Agrobacterium rhizogenes NBRC 13257
FIG|398.6.peg.6695; Rhizobium tropici
FIG|931866.6.peg.8184; Bradyrhizobium ottawaense
FIG|142585.3.peg.1658; Bradyrhizobium sp. C9
FIG|1082933.13.peg.1537; Mesorhizobium amorphae CCNWGS0123
FIG|1768789.3.peg.791; Methylobacterium sp. CCH7-A2
FIG|1381123.3.peg.3819; Aliihoeflea sp. 2WW
FIG|1297570.3.peg.1970; Mesorhizobium sp. STM 4661
FIG|935546.3.peg.3816; Mesorhizobium loti NZP2037
FIG|1128253.3.peg.1960; Bradyrhizobium japonicum CCBAU 15354
FIG|1444315.4.peg.3983; Lysobacter capsici AZ78
FIG|1185327.3.peg.1608; Xanthomonas axonopodis pv. manihotis str. Xam668
FIG|1881043.3.peg.2597; Pseudoxanthomonas sp. GM95
ALN84423.1; Lysobacter capsici
FIG|56460.15.peg.1977; Xanthomonas vesicatoria
FIG|1317116.6.peg.2759; Oceanicola sp. 22II-s10i
FIG|564137.3.peg.4320; Roseicitreum antarcticum
FIG|1952800.3.peg.3583; Rhodobacteraceae bacterium UBA2553
FIG|218673.12.peg.3041; Sulfitobacter dubius
FIG|1912092.3.peg.2119; Nioella sediminis
FIG|1736558.3.peg.5006; Ensifer sp. Root558
FIG|91360.5.peg.3717; Desulforhopalus singaporensis
FIG|1948756.3.peg.2576; Spirochaetia bacterium UBA2205
FIG|1855322.3.peg.103; Bradyrhizobium sp. Rc3b
FIG|1437360.11.peg.2429; Bradyrhizobium erythrophlei
FIG|1871052.3.peg.1026; Afipia sp.
FIG|1038860.3.peg.8756; Bradyrhizobium elkanii WSM2783
FIG|1898112.54.peg.3758; Rhodospirillaceae bacterium
FIG|1660129.3.peg.4854; Phenylobacterium sp. SCN 70-31
FIG|1482074.3.peg.4109; Hartmannibacter diazotrophicus
FIG|1970306.3.peg.552; Acidocella sp. 35-58-6
FIG|1686310.5.peg.1409; Bartonella apis
FIG|1798192.3.peg.1953; Thalassospira sp. KO164
FIG|1235461.17.peg.11; Sinorhizobium meliloti GR4
FIG|442.12.peg.222; Gluconobacter oxydans
FIG|1938607.3.peg.1954; Sphingomonas sp. LM7
FIG|1231624.3.peg.39; Asaia bogorensis NBRC 16594
FIG|1121271.3.peg.4112; Gemmobacter nectariphilus DSM 15620
FIG|33059.16.peg.1690; Acidithiobacillus caldus
FIG|502025.10.peg.925; Haliangium ochraceum DSM 14365
FIG|1734406.3.peg.691; Alphaproteobacteria bacterium BRH_c36
FIG|1979207.3.peg.4304; Parvularcula sp.
FIG|1953057.3.peg.74; Parvularculaceae bacterium UBA4496
FIG|858423.3.peg.10004; Bradyrhizobium arachidis
FIG|267128.3.peg.2015; Sphingopyxis granuli
FIG|582667.3.peg.5553; Methylobacterium pseudosasicola
FIG|1187852.3.peg.2712; Methylobacterium tarhaniae
FIG|582675.3.peg.1247; Methylobacterium gossipiicola
FIG|1951640.3.peg.515; Deferribacteraceae bacterium UBA6799
FIG|1948417.4.peg.1606; Alphaproteobacteria bacterium UBA6187
FIG|45074.5.peg.981; Legionella santicrucis
FIG|1434232.4.peg.2927; Magnetofaba australis IT-1
FIG|1945950.3.peg.3568; Acinetobacter sp. UBA6526
FIG|106654.22.peg.994; Acinetobacter nosocomialis
FIG|1977883.3.peg.3023; Acinetobacter sp. ANC 3903
FIG|1945948.3.peg.700; Acinetobacter sp. UBA5984
FIG|1226327.3.peg.2796; Acinetobacter kookii
FIG|1879049.4.peg.5949; Acinetobacter sp. WCHAc010034
FIG|1945955.3.peg.1951; Acinetobacter sp. UBA7614
FIG|1675530.3.peg.2149; Acinetobacter genomosp. 33YU
FIG|1310638.3.peg.1006; Acinetobacter baumannii 1437282
FIG|1400001.4.peg.34; Necropsobacter massiliensis
FIG|1132496.5.peg.136; Pasteurella multocida subsp. multocida str. HN06
FIG|1908263.4.peg.2604; Rodentibacter trehalosifermentans
FIG|375432.4.peg.200; Haemophilus influenzae R3021
FIG|400668.8.peg.3776; Marinomonas sp. MWYL1
FIG|1913989.193.peg.841; Gammaproteobacteria bacterium
FIG|856793.5.peg.1975; Micavibrio aeruginosavorus ARL-13
SBW23286.1; Citrobacter europaeus
FIG|1736225.3.peg.985; Erwinia sp. Leaf53
FIG|29486.12.peg.818; Yersinia ruckeri
FIG|914128.3.peg.2502; Serratia symbiotica str. Tucson
FIG|1796497.3.peg.952; Grimontia celer
FIG|1095649.3.peg.3298; Vibrio cholerae O1 str. EM-1676A
FIG|137584.4.peg.1627; Thalassomonas viridans
FIG|173990.3.peg.1773; Rheinheimera pacifica
FIG|1720343.3.peg.3189; Pseudoalteromonas sp. 1_2015MBL_MicDiv
FIG|1202962.4.peg.1481; Moritella marina ATCC 15381
FIG|669.50.peg.2993; Vibrio harveyi
FIG|691.32.peg.1517; Vibrio natriegens
FIG|156578.3.peg.2521; Alteromonadales bacterium TW-7
FIG|661.14.peg.380; Photobacterium angustum
FIG|654.94.peg.1733; Aeromonas veronii
FIG|703.9.peg.319; Plesiomonas shigelloides
FIG|589873.36.peg.1971; Alteromonas australica
FIG|28107.3.peg.3571; Pseudoalteromonas espejiana
FIG|1547444.3.peg.4264; Pseudoalteromonas sp. PLSV
FIG|629266.7.peg.847; Pseudomonas syringae pv. actinidiae str. M302091
FIG|251722.19.peg.4059; Pseudomonas amygdali pv. aesculi
FIG|587851.4.peg.1470; Pseudomonas chlororaphis subsp. aureofaciens
FIG|1265490.3.peg.2330; Pseudomonas sp. URMO17WK12:I8
FIG|316.101.peg.3534; Pseudomonas stutzeri
FIG|1916993.3.peg.4917; Pseudomonas putida
FIG|1628833.3.peg.2448; Pseudomonas sp. ES3-33
FIG|1283291.4.peg.1991; Pseudomonas sp. URMO17WK12:I11
FIG|83963.5.peg.3885; Pseudomonas syringae pv. papulans
FIG|1206777.3.peg.4334; Pseudomonas sp. Lz4W
FIG|113268.3.peg.3785; Bathymodiolus platifrons methanotrophic gill symbiont
FIG|1131284.3.peg.1562; zeta proteobacterium SCGC AB-137-C09
FIG|2026807.7.peg.2258; Zetaproteobacteria bacterium
FIG|281689.4.peg.2060; Desulfuromonas acetoxidans DSM 684
FIG|1188231.4.peg.1200; Mariprofundus ferrooxydans M34
FIG|1367489.3.peg.682; Aliivibrio fischeri SA1G
FIG|1873135.3.peg.4249; Shewanella sp. SACH
FIG|663.73.peg.2465; Vibrio alginolyticus
FIG|1588629.3.peg.1134; Aeromonas sp. L_1B5_3
FIG|1121922.3.peg.3454; Glaciecola pallidula DSM 14239 = ACAM 615
FIG|351745.9.peg.2506; Shewanella sp. W3-18-1
FIG|29497.20.peg.3798; Vibrio splendidus
FIG|1367486.3.peg.187; Aliivibrio fischeri CB37
FIG|511062.4.peg.1890; Oceanimonas sp. GK1
FIG|654.12.peg.188; Aeromonas veronii
FIG|29497.21.peg.4482; Vibrio splendidus
FIG|1659713.3.peg.560; Enterobacter bugandensis
FIG|1124991.3.peg.3617; Morganella morganii subsp. morganii KT
FIG|104623.3.peg.1381; Serratia sp. ATCC 39006
FIG|1256989.3.peg.902; Providencia alcalifaciens R90-1475
FIG|1125694.3.peg.1143; Proteus mirabilis WGLW6
FIG|574096.6.peg.2693; Pantoea allii
FIG|1095774.3.peg.2623; Pantoea ananatis PA13
FIG|869692.4.peg.2910; Escherichia coli 3003
WP_140159440.1; Escherichia coli Retron-Eco2 (Ec67)
FIG|550.437.peg.1444; Enterobacter cloacae
FIG|573.13605.peg.2600; Klebsiella pneumoniae
FIG|550.285.peg.3783; Enterobacter cloacae
FIG|1265672.3.peg.3869; Salmonella enterica subsp. enterica serovar Agona str. 70.E.05
FIG|573.10028.peg.542; Klebsiella pneumoniae
FIG|749537.3.peg.218; Escherichia coli MS 115-1
ANK06786.1; Escherichia coli O25b:H4
FIG|670.880.peg.975; Vibrio parahaemolyticus
FIG|1192730.4.peg.3; Salmonella enterica subsp. enterica serovar Kintambo
FIG|1224144.4.peg.4030; Dickeya sp. CSL RW240
FIG|568766.10.peg.2937; Dickeya sp. NCPPB 3274
FIG|1076549.3.peg.4260; Pantoea rodasii
FIG|548.102.peg.3401; Klebsiella aerogenes
FIG|630.90.peg.1795; Yersinia enterocolitica
FIG|79883.5.peg.266; Bacillus horikoshii
FIG|180861.3.peg.3762; Bacillus thuringiensis serovar sumiyoshiensis
FIG|1390.157.peg.339; Bacillus amyloliquefaciens
FIG|293386.15.peg.304; Bacillus stratosphericus
FIG|1053181.3.peg.3820; Bacillus cereus BAG2X1-3
FIG|1884375.3.peg.681; Paenibacillus sp. PDC88
FIG|334735.5.peg.923; Sporosarcina koreensis
FIG|79884.3.peg.1120; Bacillus pseudalcaliphilus
FIG|1628206.3.peg.4802; Bacillus sp. LK2
FIG|1396.1605.peg.6235; Bacillus cereus
FIG|182710.3.peg.317; Oceanobacillus iheyensis
FIG|860.10.peg.486; Fusobacterium periodonticum
FIG|1855308.3.peg.1467; Trichococcus ilyis
FIG|931626.3.peg.151; Acetobacterium woodii DSM 1030
FIG|1965575.3.peg.2547; Lachnoclostridium sp. An181
FIG|1352.2757.peg.71; Enterococcus faecium
FIG|1299895.3.peg.900; Listeria monocytogenes CFSAN002349
FIG|53346.29.peg.1591; Enterococcus mundtii
FIG|1649188.10.peg.1545; Listeria goaensis
FIG|158847.6.peg.432; Megamonas hypermegale
FIG|1121289.3.peg.2775; Clostridiisalibacter paucivorans DSM 22131
FIG|1950885.3.peg.858; Clostridiales bacterium UBA4693
FIG|1965576.3.peg.1978; Pseudoflavonifractor sp. An184
FIG|1952416.3.peg.1629; Ruminococcaceae bacterium UBA642
FIG|1262803.3.peg.8; Clostridium sp. CAG:413
FIG|28037.216.peg.60; Streptococcus mitis
FIG|1074052.3.peg.33; Streptococcus sobrinus TCI-9
FIG|1304.207.peg.1536; Streptococcus salivarius
FIG|1154859.3.peg.955; Streptococcus agalactiae LMG 14609
FIG|1080071.3.peg.332; Streptococcus orisasini
FIG|1139219.3.peg.2194; Enterococcus dispar ATCC 51266
FIG|1834176.3.peg.811; Enterococcus sp. 3G1_DIV0629
FIG|1622.15.peg.947; Lactobacillus murinus
FIG|565651.6.peg.1942; Enterococcus faecalis ARO1/DG
FIG|1473546.3.peg.703; Lysinibacillus sp. BF-4
FIG|37734.13.peg.137; Enterococcus casseliflavus
FIG|492670.92.peg.623; Bacillus velezensis
FIG|1639.1907.peg.2641; Listeria monocytogenes
FIG|1123489.3.peg.170; Veillonella magna DSM 19857
FIG|1280687.3.peg.1880; Butyrivibrio fibrisolvens YRB2005
FIG|1262889.3.peg.680; Eubacterium sp. CAG:38
FIG|1235800.3.peg.2226; Lachnospiraceae bacterium 10-1
FIG|1897035.3.peg.445; Firmicutes bacterium CAG:552_39_19
FIG|199.588.peg.774; Campylobacter concisus
FIG|1111133.4.peg.219; Peptoniphilus sp. BV3AC2
FIG|936589.3.peg.875; Veillonella sp. AS16
WP_070600378.1;
FIG|1896998.3.peg.1750; Coprococcus sp. CAG:131-related_45_246
FIG|41170.3.peg.3013; Exiguobacterium acetylicum
FIG|59620.44.peg.897; uncultured Clostridium sp.
FIG|1262843.3.peg.313; Clostridium sp. CAG:813
FIG|1262834.3.peg.1287; Clostridium sp. CAG:715
FIG|1256219.3.peg.760; Lactobacillus paracasei subsp. paracasei Lpp230
FIG|115778.31.peg.1994; Leuconostoc gelidum subsp. gasicomitatum
FIG|29385.174.peg.531; Staphylococcus saprophyticus
FIG|1295.21.peg.75; Staphylococcus schleiferi
FIG|148814.13.peg.1360; Lactobacillus kunkeei
FIG|1282.1242.peg.673; Staphylococcus epidermidis
FIG|1581078.3.peg.1186; Staphylococcus sp. HMSC10C03
FIG|1891097.3.peg.280; Macrococcus goetzii
WP_080703103.1;
FIG|1214184.3.peg.1129; Streptococcus suis 22083
FIG|1154771.3.peg.209; Streptococcus agalactiae FSL C1-487
FIG|1415765.3.peg.1578; Streptococcus mitis 21/39
FIG|1581074.3.peg.720; Granulicatella sp. HMSC31F03
FIG|1349.233.peg.712; Streptococcus uberis
FIG|1946281.3.peg.392; Catabacter sp. UBA5893
FIG|1328309.5.peg.1889; Lactobacillus plantarum IPLA88
FIG|1214190.3.peg.2034; Streptococcus suis YS17
FIG|29385.135.peg.2098; Staphylococcus saprophyticus
FIG|1715184.3.peg.1265; Aerococcus sp. HMSC035B07
FIG|1881068.3.peg.2940; Sphingomonas sp. OV641
FIG|1522072.3.peg.3829; Sphingobium sp. ba1
FIG|1802172.3.peg.237; Sphingopyxis sp. RIFCSPHIGHO2_12_FULL_65_19
FIG|1128204.3.peg.2189; Bradyrhizobium elkanii CCBAU 43297
FIG|1708715.5.peg.4517; Ensifer aridi
FIG|195105.3.peg.2062; Haematobacter massiliensis
FIG|1283312.3.peg.4182; Sphingomonas wittichii DC-6
FIG|1120654.4.peg.406; Ensifer sp. LC499
FIG|529.36.peg.3144; Ochrobactrum anthropi
FIG|1194716.3.peg.4774; Sinorhizobium meliloti AK75
FIG|1660088.4.peg.2967; Agrobacterium sp. SCN 61-19
FIG|1951259.3.peg.2515; Sphingomonadales bacterium UBA6174
FIG|1912891.5.peg.2102; Sphingobium sp.
FIG|1670800.3.peg.1844; Mesorhizobium oceanicum
FIG|2032658.3.peg.157; Alphaproteobacteria bacterium WMHbin7
FIG|1819565.5.peg.2208; Flavimaricola marinus
FIG|1245469.3.peg.1160; Bradyrhizobium oligotrophicum S58
FIG|1615890.4.peg.173; Bradyrhizobium sp. LTSP849
FIG|56454.3.peg.3464; Xanthomonas hortorum
FIG|40324.384.peg.1060; Stenotrophomonas maltophilia
FIG|1801972.3.peg.1832; Planctomycetes bacterium RBG_19FT_COMBO_48_8
FIG|1978765.3.peg.3488; Nitrospira sp. ST-bin5
FIG|2009322.3.peg.2770; Leptolyngbya ohadii IS1
FIG|1325564.3.peg.3733; Nitrospira japonica
FIG|43662.9.peg.1688; Pseudoalteromonas piscicida
FIG|670.134.peg.4439; Vibrio parahaemolyticus
FIG|998520.3.peg.3325; Pseudoalteromonas agarivorans
FIG|1723759.3.peg.401; Pseudoalteromonas sp. P1-26
FIG|672.133.peg.585; Vibrio vulnificus
FIG|1324960.19.peg.585; Aeromonas salmonicida subsp. pectinolytica 34mel
FIG|196024.16.peg.3965; Aeromonas dhakensis
FIG|654.27.peg.4266; Aeromonas veronii
FIG|1802253.3.peg.1045; Sulfurimonas sp. RIFCSPLOWO2_12_36_12
FIG|636.16.peg.3905; Edwardsiella tarda
FIG|1124958.3.peg.5012; Salmonella enterica subsp. enterica serovar Muenster str. 0315
FIG|573.10007.peg.225; Klebsiella pneumoniae
FIG|1946737.3.peg.4002; Leclercia sp. UBA1284
FIG|1398203.3.peg.3712; Xenorhabdus bovienii str. kraussei Quebec
FIG|615.247.peg.2151; Serratia marcescens
FIG|52441.3.peg.3752; Nitrosomonas aestuarii
FIG|1951948.3.peg.242; Hyphomonadaceae bacterium UBA2389
FIG|165186.29.peg.27; uncultured Ruminococcus sp.
FIG|2013842.3.peg.1881; Synergistetes bacterium HGW-Synergistetes-1
FIG|411484.7.peg.436; Clostridium sp. SS2/1
FIG|460384.4.peg.447; Enterocloster lavalensis
FIG|1761781.3.peg.2961; Clostridium sp. DSM 8431
FIG|1451.25.peg.614; Paenibacillus amylolyticus
FIG|1776378.3.peg.2009; Paenibacillus phocaensis
FIG|1866315.3.peg.2122; Bacillus sp. N35-10-4
FIG|1034836.4.peg.4077; Bacillus amyloliquefaciens XH7
FIG|1397.14.peg.5097; Bacillus circulans
FIG|1497681.5.peg.772; Listeria newyorkensis
FIG|1053224.3.peg.4333; Bacillus cereus VD021
FIG|1374.4.peg.2798; Planococcus kocurii
FIG|458233.11.peg.419; Macrococcus caseolyticus JCSC5402
FIG|417368.6.peg.944; Enterococcus thailandicus
FIG|1353.16.peg.736; Enterococcus gallinarum
FIG|1639.1307.peg.2578; Listeria monocytogenes
FIG|1649188.4.peg.450; Listeria goaensis
FIG|333990.5.peg.1279; Carnobacterium sp. AT7
FIG|1121085.3.peg.4805; Bacillus aidingensis DSM 18341
FIG|659243.6.peg.1163; Bacillus siamensis
FIG|1965645.3.peg.1428; Alistipes sp. An54
FIG|1950664.3.peg.363; Bacteroidales bacterium UBA5918
FIG|681398.3.peg.1596; Paludibacter jiangxiensis
FIG|1947481.3.peg.1596; Sphingobacterium sp. UBA1498
FIG|1946424.3.peg.2345; Dysgonomonas sp. UBA4861
FIG|188932.3.peg.968; Pedobacter cryoconitis
FIG|505249.7.peg.1802; Arcobacter marinus
FIG|1802259.3.peg.374; Sulfurimonas sp. RIFOXYD12_FULL_33_39
FIG|1872629.13.peg.663; Arcobacter sp.
FIG|497650.4.peg.949; Sulfurovum sp. enrichment culture clone C5
FIG|1981711.3.peg.707; Pseudomonas sp. B8(2017)
FIG|287.926.peg.3808; Pseudomonas aeruginosa
FIG|157782.3.peg.183; Pseudomonas parafulva
FIG|1225174.5.peg.576; Pseudomonas mendocina S5.2
FIG|237610.8.peg.4301; Pseudomonas psychrotolerans
FIG|1116369.3.peg.182; Hoeflea sp. 108
WP_080858354.1;
FIG|1679460.3.peg.2715; Marinibacterium profundimaris
FIG|1811547.3.peg.510; Maritimibacter sp. REDSEA-S28_B5
FIG|93684.8.peg.518; Roseivivax halotolerans
EMZ69714.1; Escherichia coli 174900
FIG|103796.87.peg.3165; Pseudomonas syringae pv. actinidiae
WP_078828851.1; Pantoea ananatis
FIG|2018067.3.peg.1734; Pseudomonas sp. FDAARGOS_380
FIG|294.255.peg.5151; Pseudomonas fluorescens
FIG|287.4271.peg.5445; Pseudomonas aeruginosa
FIG|46677.3.peg.3237; Pseudomonas agarici
FIG|83964.10.peg.849; Pseudomonas coronafaciens pv. porri
FIG|1932113.4.peg.2793; Pseudomonas sp. PA1(2017)
FIG|1712677.3.peg.189; Pseudomonas sp. 2822-15
FIG|1479235.3.peg.2741; Halomonas sp. HL-48
FIG|227946.12.peg.3247; Xanthomonas translucens pv. poae
FIG|40324.220.peg.2801; Stenotrophomonas maltophilia
FIG|227946.13.peg.35; Xanthomonas translucens pv. poae
FIG|487909.15.peg.4212; Xanthomonas translucens pv. undulosa
FIG|40324.145.peg.2120; Stenotrophomonas maltophilia
FIG|1182783.3.peg.8; Xanthomonas campestris JX
FIG|1736581.3.peg.4144; Lysobacter sp. Root667
FIG|470.4256.peg.2128; Acinetobacter baumannii
FIG|1804984.3.peg.4735; Burkholderia sp. OLGA172
FIG|1882792.3.peg.5959; Burkholderia sp. CF145
FIG|1458357.5.peg.7849; Caballeronia jiangsuensis
FIG|674703.3.peg.3992; Rhodoplanes sp. Z2-YC6860
FIG|1230476.3.peg.595; Bradyrhizobium sp. DFCI-1
FIG|1752222.3.peg.1730; Rhizobiales bacterium Ga0077525
FIG|1948848.3.peg.320; Patescibacteria group bacterium UBA6220
FIG|1860092.3.peg.3966; Alphaproteobacteria bacterium MedPE-SWcel
FIG|398.7.peg.3301; Rhizobium tropici
FIG|418630.3.peg.960; Rhodobacter megalophilus
FIG|56.40.peg.5712; Sorangium cellulosum
FIG|1660160.3.peg.2510; Acidobacteria bacterium SCN 69-37
FIG|1661042.3.peg.2224; Pseudomonas sp. NBRC 111127
FIG|1712678.3.peg.4198; Pseudomonas sp. 2822-17
FIG|1736561.3.peg.128; Pseudomonas sp. Root562
FIG|76760.8.peg.1730; Pseudomonas rhodesiae
FIG|1295133.4.peg.7170; Pseudomonas putida JCM 18798
FIG|1718917.3.peg.3132; Pseudomonas sp. ICMP 460
FIG|237306.3.peg.591; Pseudomonas syringae pv. persicae
FIG|1079060.3.peg.1479; Pseudomonas savastanoi pv. phaseolicola 1644R
FIG|1981714.3.peg.1068; Pseudomonas sp. B5(2017)
FIG|1419583.3.peg.4516; Pseudomonas mandelii PD30
FIG|1718918.3.peg.4166; Pseudomonas sp. ICMP 561
FIG|64988.7.peg.76; Alcanivorax jadensis
FIG|1961564.3.peg.685; Desulfovibrionaceae bacterium UBA5546
FIG|2004648.3.peg.1747; Acinetobacter sp. WCHA39
FIG|1080187.3.peg.399; Cupriavidus sp. UYPR2.512
FIG|76114.8.peg.258; Aromatoleum aromaticum EbN1
FIG|196367.9.peg.6286; Caballeronia sordidicola
FIG|1217418.3.peg.694; Cupriavidus sp. HPC(L)
FIG|1752216.3.peg.4007; Nitrosomonadales bacterium Ga0074132
FIG|1249621.3.peg.3614; Cupriavidus sp. HMR-1
FIG|179879.8.peg.6514; Burkholderia anthina
FIG|1246301.3.peg.4482; Variovorax paradoxus B4
WP_092746164.1; Acidovorax valerianellae
FIG|536.30.peg.857; Chromobacterium violaceum
FIG|1961112.3.peg.115; Planctomycetes bacterium UTPLA1
FIG|44574.5.peg.4575; Nitrosomonas communis
FIG|265901.4.peg.190; Photobacterium sp. J15
FIG|80852.21.peg.1289; Aliivibrio wodanis
FIG|1136159.3.peg.2534; Vibrio cyclitrophicus 1F111
FIG|24.6.peg.4539; Shewanella putrefaciens
FIG|888433.3.peg.1974; Pseudoalteromonas sp. GutCa3
FIG|196024.6.peg.3651; Aeromonas dhakensis
FIG|1352943.3.peg.5028; Vibrio harveyi E385
WP_088124663.1; Vibrio cholerae
FIG|29497.15.peg.1220; Vibrio splendidus
FIG|670.413.peg.1227; Vibrio parahaemolyticus
FIG|584.91.peg.3337; Proteus mirabilis
FIG|263819.5.peg.201; Yersinia aleksiciae
FIG|1656094.3.peg.1449; Alteromonas confluentis
FIG|634.5.peg.607; Yersinia bercovieri
FIG|630.85.peg.4080; Yersinia enterocolitica
FIG|1212491.3.peg.1847; Legionella fallonii LLAP-10
FIG|1498499.3.peg.2812; Legionella norrlandica
FIG|1844092.4.peg.3143; Pseudomonas sp. 8 R 14
FIG|1441629.3.peg.2119; Pseudomonas cichorii JBC1
WP_092369835.1; Pseudomonas seleniipraecipitans
FIG|477228.3.peg.2040; Pseudomonas stutzeri TS44
FIG|317.249.peg.4299; Pseudomonas syringae
FIG|1597.16.peg.2055; Lactobacillus paracasei
FIG|1184720.6.peg.2708; Rhizobium anhuiense
FIG|1566263.3.peg.185; Rhizobium sp. NFR03
FIG|1951216.3.peg.1622; Rhizobiales bacterium UBA1909
FIG|1219052.3.peg.3572; Sphingomonas pruni NBRC 15498
FIG|376620.8.peg.122; Gluconobacter japonicus
FIG|1736587.3.peg.2638; Devosia sp. Root685
WP_011269850.1; Xanthomonas campestris
FIG|1195246.3.peg.467; Alishewanella agri BL06
FIG|1931276.3.peg.1294; Haliangium sp. UPWRP_2
FIG|1931204.4.peg.12; Confluentimicrobium sp.
FIG|2052957.3.peg.3497; Pseudorhodobacter sp. MZDSW-24AT
FIG|1952825.3.peg.2772; Rhodobiaceae bacterium UBA4205
FIG|1189622.3.peg.1716; Pseudomonas amygdali pv. tabaci str. 6605
FIG|294.173.peg.2741; Pseudomonas fluorescens
FIG|294.122.peg.3307; Pseudomonas fluorescens
FIG|1198456.3.peg.4053; Pseudomonas guguanensis
FIG|1855380.3.peg.1951; Pseudomonas sp. Z003-0.4C(8344-21)
FIG|1144330.3.peg.3879; Pseudomonas sp. GM48
FIG|86265.3.peg.2799; Pseudomonas thivervalensis
FIG|1881035.3.peg.3817; Mitsuaria sp. PDC51
FIG|511.8.peg.774; Alcaligenes faecalis
FIG|1095552.3.peg.2955; Methylobacter luteus IMV-B-3098
FIG|1690268.3.peg.1172; Acidovorax sp. SD340
FIG|871652.3.peg.1451; Poseidonocella sedimentorum
FIG|1946868.3.peg.175; Methylophaga sp. UBA1490
FIG|1924940.3.peg.1147; Mameliella sp.
FIG|1912891.7.peg.5370; Sphingobium sp.
FIG|1236503.3.peg.1539; Acetobacter persici JCM 25330
FIG|1745182.3.peg.1942; Paracoccus sp. MKU1
FIG|1112.5.peg.2875; Porphyrobacter neustonensis
WP_051585410.1; Sphingomonas paucimobilis
FIG|1082931.4.peg.3584; Pelagibacterium halotolerans B2
FIG|1907665.3.peg.5475; Agrobacterium sp. DSM 25558
FIG|1841652.4.peg.3782; Agrobacterium sp. 13-626
FIG|1736312.3.peg.3441; Rhizobium sp. Leaf262
FIG|1768770.3.peg.4687; Caulobacter sp. CCH5-E12
FIG|355591.9.peg.1867; Marinobacter vinifirmus
FIG|1869214.4.peg.1848; Rheinheimera sp.
FIG|1946470.3.peg.3546; Erythrobacter sp. UBA2510
FIG|1860090.3.peg.231; Roseobacter sp. MedPE-SWde
FIG|2020902.8.peg.1814; Ponticaulis sp.
FIG|940286.3.peg.3612; Komagataeibacter oboediens 174Bp2
FIG|1736380.3.peg.1842; Rhizobium sp. Leaf453
FIG|665126.3.peg.2283; Prosthecomicrobium hirschii
FIG|2029410.3.peg.1956; Mesorhizobium sp. WSM4311
WP_003169203.1; Brevundimonas diminuta
FIG|1884373.3.peg.3317; Mesorhizobium sp. YR577
FIG|989436.3.peg.3203; Pseudovibrio sp. Ad5
FIG|1736359.3.peg.3976; Rhizobium sp. Leaf386
FIG|104102.12.peg.3797; Acetobacter tropicalis
FIG|1500305.3.peg.4736; Rhizobium sp. OK665
FIG|1842535.30.peg.6; Blastomonas sp. RAC04
FIG|70775.16.peg.91; Pseudomonas plecoglossicida
FIG|287.4262.peg.2063; Pseudomonas aeruginosa
WP_017702484.1; Pseudomonas syringae
FIG|1357292.3.peg.4700; Pseudomonas syringae pv. pisi str. PP1
FIG|287.2436.peg.3554; Pseudomonas aeruginosa
FIG|76758.3.peg.4722; Pseudomonas orientalis
FIG|1904755.3.peg.3469; Pseudomonas sp. 43NM1
FIG|47879.37.peg.693; Pseudomonas corrugata
FIG|1771311.3.peg.1935; Pseudomonas sp. ATCC PTA-122608
FIG|2008975.3.peg.1976; Pseudomonas sp. Irchel 3E13
FIG|1259798.3.peg.1121; Pseudomonas sp. LAMO17WK12:I2
FIG|1736487.3.peg.2103; Noviherbaspirillum sp. Root189
FIG|1706231.5.peg.1557; Janthinobacterium sp. CG23_2
FIG|1804984.3.peg.4700; Burkholderia sp. OLGA172
FIG|54067.3.peg.2925; Xylophilus ampelinus
FIG|40324.357.peg.1426; Stenotrophomonas maltophilia
FIG|1967657.4.peg.913; Salmonella enterica subsp. enterica serovar Telelkebir
FIG|573.14330.peg.816; Klebsiella pneumoniae
FIG|615.357.peg.16; Serratia marcescens
FIG|1122616.3.peg.231; Oceanospirillum beijerinckii DSM 7166
FIG|314276.4.peg.1389; Idiomarina baltica OS145
FIG|1038921.4.peg.2790; Pseudomonas chlororaphis subsp. aureofaciens 30-84
FIG|292.72.peg.3280; Burkholderia cepacia
FIG|1899355.18.peg.947; Oceanospirillaceae bacterium
FIG|2015356.3.peg.5401; Burkholderia sp. AU33647
FIG|206665.3.peg.1731; Desulfonauticus submarinus
FIG|1987165.3.peg.2564; Sphingobium sp. GW456-12-10-14-TSB1
FIG|1283312.3.peg.2182; Sphingomonas wittichii DC-6
FIG|1223566.3.peg.1810; Bradyrhizobium sp. CCGE-LA001
FIG|76761.16.peg.744; Pseudomonas veronii
PIY00499.1; Hydrogenophilales bacterium CG_4_10_14_3_um_filter_58_23
FIG|305.94.peg.4778; Ralstonia solanacearum
FIG|1758178.5.peg.2546; Celeribacter ethanolicus
FIG|1354263.4.peg.2524; Hafnia paralvei ATCC 29927
FIG|1125979.3.peg.1941; Rhizobium sp. PDO1-076
FIG|1338032.3.peg.3393; Vibrio parahaemolyticus O1:K33 str. CDC_K4557
FIG|1898112.54.peg.3344; Rhodospirillaceae bacterium
FIG|1432558.3.peg.4265; Klebsiella pneumoniae ISC21
FIG|333962.3.peg.2767; Providencia heimbachae
FIG|60552.10.peg.2414; Burkholderia vietnamiensis
WP_011808964.1; Verminephrobacter eiseniae
FIG|1844107.4.peg.2966; Pseudomonas sp. 58 R 12
FIG|1952916.3.peg.906; Synergistaceae bacterium UBA5549
FIG|458817.8.peg.542; Shewanella halifaxensis HAW-EB4
FIG|1674859.3.peg.1291; Spirochaetales bacterium Spiro_03
FIG|1121434.3.peg.22; Halodesulfovibrio aestuarii DSM 10141
FIG|1262899.3.peg.286; Fusobacterium sp. CAG:439
FIG|57320.3.peg.1084; Pseudodesulfovibrio profundus
FIG|1736444.3.peg.3753; Acinetobacter sp. Root1280
FIG|1310670.3.peg.2122; Acinetobacter sp. 907131
FIG|505345.6.peg.150; Gallibacterium genomosp. 3
FIG|670.887.peg.4391; Vibrio parahaemolyticus
FIG|196024.16.peg.2750; Aeromonas dhakensis
FIG|663.48.peg.1321; Vibrio alginolyticus
FIG|28141.133.peg.4717; Cronobacter sakazakii
FIG|1117315.3.peg.3; Pseudoalteromonas haloplanktis ATCC 14393
FIG|1917164.4.peg.2739; Shewanella sp. UCD-KL21
FIG|2006083.3.peg.3222; Photobacterium sp. CECT 9192
FIG|584.227.peg.2146; Proteus mirabilis
FIG|1792834.4.peg.1793; Marinicella sediminis
FIG|1333513.3.peg.3775; Pseudoalteromonas haloplanktis TAE56
FIG|1305826.3.peg.1246; Streptomyces sp. Amel2xC10
WP_048809063.1; Microbacterium ginsengisoli
FIG|1987376.3.peg.4246; Pseudonocardia sp. N23
FIG|164115.3.peg.6832; Streptomyces niveiscabiei
FIG|285676.33.peg.4994; Micromonospora saelicesensis
SFF52649.1; Streptomyces alni
FIG|1100822.3.peg.6408; Streptomyces sp. AmelKG-E11A
WP_098467790.1;
FIG|1190417.3.peg.2916; Geodermatophilus telluris
SDS16714.1; Agrococcus carbonis
FIG|692370.5.peg.1108; Altererythrobacter dongtanensis
FIG|1736370.3.peg.1383; Sphingomonas sp. Leaf412
FIG|1759074.3.peg.2615; Sphingopyxis sp. HIX
FIG|1120928.3.peg.922; Acinetobacter tjernbergiae DSM 14971 = CIP 107465
FIG|470.4268.peg.2032; Acinetobacter baumannii
FIG|1217627.3.peg.995; Acinetobacter baumannii NIPH 67
FIG|28450.149.peg.5786; Burkholderia pseudomallei
FIG|2032650.3.peg.3543; Magnetococcales bacterium HCHbin5
FIG|101571.169.peg.3909; Burkholderia ubonensis
FIG|396597.7.peg.2128; Burkholderia ambifaria MEX-5
FIG|869212.3.peg.3514; Turneriella parva DSM 21527
FIG|1196083.80.peg.1885; Snodgrassella alvi
FIG|1304886.3.peg.1257; Desulfotignum balticum DSM 7044
FIG|555.16.peg.1362; Pectobacterium carotovorum subsp. carotovorum
FIG|1421338.3.peg.151; Enterobacter asburiae L1
FIG|443144.3.peg.785; Geobacter sp. M21
FIG|1265503.3.peg.1271; Colwellia piezophila ATCC BAA-637
FIG|55601.100.peg.1601; Vibrio anguillarum
FIG|299766.9.peg.4890; Enterobacter hormaechei subsp. steigerwaltii
FIG|243231.5.peg.1360; Geobacter sulfurreducens PCA
FIG|1263083.3.peg.558; Klebsiella variicola CAG:634
FIG|57706.9.peg.1106; Citrobacter braakii
FIG|1619244.3.peg.1323; Enterobacter bugandensis
SHO56340.1; Vibrio quintilis
FIG|688.15.peg.906; Aliivibrio logei
FIG|663.75.peg.4800; Vibrio alginolyticus
FIG|1967612.3.peg.4508; Salmonella enterica subsp. houtenae serovar 50:z4, z23:—
FIG|82985.3.peg.3508; Pragia fontium
FIG|55207.5.peg.1840; Pectobacterium betavasculorum
FIG|582.25.peg.388; Morganella morganii
FIG|1006598.5.peg.80; Serratia plymuthica RVH1
FIG|82977.3.peg.3268; Buttiauxella agrestis
CRY53703.1; Yersinia intermedia
FIG|595494.3.peg.706; Tolumonas auensis DSM 9187
FIG|1217694.3.peg.3300; Acinetobacter sp. CIP 64.2
FIG|470.2679.peg.715; Acinetobacter baumannii
FIG|1879050.4.peg.2797; Acinetobacter wuhouensis
FIG|2004650.3.peg.1818; Acinetobacter chinensis
FIG|648.80.peg.1405; Aeromonas caviae
FIG|1217722.3.peg.1866; Pseudomonas sp. S13.1.2
FIG|294.88.peg.4234; Pseudomonas fluorescens
FIG|629262.5.peg.1917; Pseudomonas syringae pv. japonica str. M301072
WP_053932309.1; Pseudomonas coronafaciens
FIG|1844101.3.peg.4702; Pseudomonas sp. 31 R 17
FIG|380021.13.peg.6149; Pseudomonas protegens
FIG|287.3716.peg.2163; Pseudomonas aeruginosa
FIG|287.3208.peg.1464; Pseudomonas aeruginosa
FIG|1952221.3.peg.555; Methylococcaceae bacterium UBA2780
FIG|1869214.3.peg.3542; Rheinheimera sp.
FIG|375286.7.peg.830; Janthinobacterium sp. Marseille
FIG|536.26.peg.4616; Chromobacterium violaceum
FIG|983548.3.peg.2977; Dokdonia sp. 4H-3-7-5
FIG|307480.5.peg.1780; Chryseobacterium vrystaatense
FIG|1262921.3.peg.2213; Prevotella sp. CAG:1185
FIG|1965649.3.peg.4193; Butyricimonas sp. An62
FIG|1951558.3.peg.3731; Chitinophagaceae bacterium UBA4411
FIG|1950669.3.peg.2035; Bacteroidales bacterium UBA6192
FIG|1869230.3.peg.3025; Chryseobacterium sp. CBo1
FIG|1500294.3.peg.2814; Chryseobacterium sp. YR485
FIG|1756149.11.peg.2545; Elizabethkingia bruuniana
FIG|1137281.3.peg.1436; Xanthomarina gelatinilytica
FIG|1964365.5.peg.2525; Sneathiella sp.
FIG|28450.428.peg.5049; Burkholderia pseudomallei
FIG|1628751.3.peg.813; Nostoc linckia z16
FIG|60137.10.peg.1138; Sulfitobacter pontiacus
FIG|1580596.3.peg.2701; Phaeobacter piscinae
FIG|1041141.4.peg.4741; Rhizobium leguminosarum bv. viciae 128C53
WP_063290764.1; unclassified Pseudovibrio
FIG|1816219.4.peg.1873; Colwellia sp. PAMC 21821
FIG|651.3.peg.13; Aeromonas media
FIG|134375.17.peg.3222; Achromobacter sp.
WP_011296194.1; Cupriavidus pinatubonensis
FIG|1513890.4.peg.2322; Pseudomonas chlororaphis subsp. piscium
FIG|294.193.peg.980; Pseudomonas fluorescens
FIG|287.2516.peg.114; Pseudomonas aeruginosa
FIG|2006083.3.peg.3219; Photobacterium sp. CECT 9192
FIG|458817.8.peg.538; Shewanella halifaxensis HAW-EB4
FIG|1073383.3.peg.1289; Aeromonas veronii AMC34
FIG|190893.14.peg.2110; Vibrio coralliilyticus
FIG|663.144.peg.2659; Vibrio alginolyticus
FIG|55601.106.peg.926; Vibrio anguillarum
FIG|669.51.peg.5531; Vibrio harveyi
FIG|1250059.5.peg.3511; Tenacibaculum sp. MAR_2009_124
FIG|906888.6.peg.2449; Nonlabens ulvanivorans
WP_042276051.1; Nonlabens sediminis
FIG|1953167.3.peg.1254; Bacteroidetes bacterium UBA6221
FIG|991.14.peg.629; Flavobacterium hydatis
FIG|1121890.3.peg.5; Flavobacterium frigidarium DSM 17623
FIG|387094.4.peg.1115; Flavobacterium hercynium
FIG|1946558.3.peg.2413; Flavobacterium sp. UBA7665
FIG|253.27.peg.2882; Chryseobacterium indologenes
FIG|1685010.5.peg.4376; Chryseobacterium glaciei
FIG|1500289.3.peg.4103; Chryseobacterium sp. OV705
FIG|1500298.3.peg.2823; Chryseobacterium sp. YR561
FIG|1797331.3.peg.2180; Bacteroidetes bacterium GWE2_29_8
FIG|1947498.3.peg.1366; Sphingobacterium sp. UBA4616
WP_074239321.1; Chitinophaga niabensis
FIG|192149.7.peg.174; Muricauda sp.
FIG|718222.3.peg.4924; Bacillus cereus TIAC219
FIG|1053210.3.peg.211; Bacillus cereus HuB4-10
FIG|2026089.3.peg.6077; Paenibacillus sp. XY044
FIG|1938610.3.peg.3678; Flavobacterium sp. LM5
FIG|1947482.3.peg.632; Sphingobacterium sp. UBA1575
FIG|1948844.3.peg.823; Patescibacteria group bacterium UBA6130
FIG|986.7.peg.83; Flavobacterium johnsoniae
FIG|1950382.3.peg.461; Bacteroidales bacterium UBA1181
FIG|1947145.3.peg.376; Prevotella sp. UBA3765
FIG|1122989.3.peg.367; Prevotella oris DSM 18711 = JCM 12252
FIG|1896974.3.peg.2001; Bacteroides sp. 43_108
FIG|2014804.3.peg.4306; Lewinellaceae bacterium SD302
FIG|1428.517.peg.2380; Bacillus thuringiensis
FIG|1428.574.peg.3351; Bacillus thuringiensis
FIG|1428.590.peg.5685; Bacillus thuringiensis
FIG|720554.3.peg.188; Hungateiclostridium clariflavum DSM 19732
FIG|1122203.4.peg.2283; Marinococcus halotolerans DSM 16375
FIG|1462525.3.peg.3489; Thalassobacillus sp. TM-1
FIG|1395513.3.peg.363; Sporolactobacillus laevolacticus DSM 442
FIG|1262834.3.peg.1229; Clostridium sp. CAG:715
SCI87282.1; uncultured Roseburia sp.
FIG|1952116.3.peg.2349; Lachnospiraceae bacterium UBA6480
WP_069150959.1; Lachnospiraceae
FIG|1265.10.peg.3100; Ruminococcus flavefaciens
FIG|1120998.3.peg.2858; Anaerovorax odorimutans DSM 5092
WP_072702499.1; Butyrivibrio hungatei
FIG|1232453.3.peg.2795; Clostridiales bacterium VE202-21
FIG|39485.11.peg.251; Lachnospira eligens
WP_072832325.1;
FIG|1509.24.peg.2487; Clostridium sporogenes
SCI88558.1; uncultured Clostridium sp.
FIG|1490.6.peg.2635; Paraclostridium bifermentans
FIG|1947399.3.peg.1322; Hungateiclostridiaceae bacterium UBA3548
FIG|1953138.3.peg.796; Bacteroidetes bacterium UBA1312
FIG|1950875.3.peg.364; Clostridiales bacterium UBA4139
FIG|1396.1518.peg.4860; Bacillus cereus
FIG|1305675.3.peg.2174; Bacillus solimangrovi
FIG|1423.436.peg.4365; Bacillus subtilis
FIG|361277.6.peg.1539; Terribacillus saccharophilus
FIG|1392.356.peg.4724; Bacillus anthracis
FIG|1053189.3.peg.4219; Bacillus cereus BAG5X1-1
WP_079442297.1; Clostridium chromiireducens
FIG|1953262.3.peg.783; Candidatus Omnitrophica bacterium UBA1562
FIG|1797955.3.peg.2304; Elusimicrobia bacterium RIFOXYA12_FULL_51_18
FIG|1953111.3.peg.2436; Acidobacteria bacterium UBA7540
WP_099010551.1; Escherichia coli Retron-Eco1 (Ec86)
FIG|1005565.3.peg.1153; Escherichia coli 3006
FIG|158822.8.peg.1905; Cedecea neteri
FIG|1444060.3.peg.4830; Escherichia coli 4-203-08_S1_C1
FIG|29484.22.peg.4571; Yersinia frederiksenii
FIG|529823.3.peg.332; Cellvibrio sp. OA-2007
FIG|48296.218.peg.3142; Acinetobacter pittii
FIG|550.518.peg.3445; Enterobacter cloacae
FIG|204773.6.peg.4; Herminiimonas arsenicoxydans
FIG|670.190.peg.4348; Vibrio parahaemolyticus
FIG|44577.7.peg.209; Nitrosomonas ureae
FIG|1125747.3.peg.1; Paraglaciecola agarilytica NO2
FIG|1338034.3.peg.2437; Vibrio parahaemolyticus O1:Kuk str. FDA_R31
FIG|1952844.3.peg.2619; Rhodocyclaceae bacterium UBA5533
FIG|1288788.3.peg.2384; Vibrio parahaemolyticus 3631
FIG|644.31.peg.975; Aeromonas hydrophila
FIG|498292.3.peg.28; Flavobacterium swingsii
FIG|1948088.3.peg.4515; Firmicutes bacterium UBA6132
FIG|1408433.3.peg.3094; Crocinitomix catalasitica ATCC 23190
WP_074236572.1; Chryseobacterium zeae
FIG|1127353.3.peg.1738; Salmonella enterica subsp. enterica serovar Newport str. #11-4
FIG|1881110.4.peg.120; Pantoea sesami
FIG|34038.6.peg.27; Rahnella aquatilis
FIG|630.95.peg.4256; Yersinia enterocolitica
FIG|149387.11.peg.1139; Salmonella enterica subsp. enterica serovar Brandenburg
FIG|1343738.3.peg.2232; Vibrio cholerae 2012EL-1759 Retron-Vch3 (Vc137)
FIG|1423.175.peg.4339; Bacillus subtilis
FIG|2021695.3.peg.3399; Bacillus sp. 7894-2
FIG|189426.10.peg.597; Paenibacillus odorifer
FIG|2020949.3.peg.856; Romboutsia weinsteinii
FIG|1243664.3.peg.1004; Bacillus massiliogorillae
FIG|1855345.3.peg.2971; Bacillus sp. RRD69
FIG|1946358.3.peg.2514; Clostridium sp. UBA4108
FIG|1520.90.peg.2502; Clostridium beijerinckii
FIG|79672.3.peg.288; Bacillus thuringiensis serovar medellin
FIG|189426.19.peg.3973; Paenibacillus odorifer
FIG|1497.3.peg.4049; Clostridium formicaceticum
FIG|169760.4.peg.4269; Paenibacillus stellifer
WP_073588670.1; Anaerocolumna xylanovorans
FIG|1950815.3.peg.1585; Clostridiales bacterium UBA1341
FIG|1897004.3.peg.2166; Eubacterium sp. 45_250
FIG|1946293.3.peg.290; Catabacter sp. UBA7571
FIG|1796620.3.peg.3489; Acutalibacter muris
FIG|76857.53.peg.2245; Fusobacterium nucleatum subsp. polymorphum
FIG|2013784.3.peg.1260; Firmicutes bacterium HGW-Firmicutes-3
FIG|79884.3.peg.1106; Bacillus pseudalcaliphilus
FIG|135461.47.peg.1552; Bacillus subtilis subsp. subtilis
WP_016122013.1; Bacillus cereus group
FIG|1499688.3.peg.3214; Bacillus sp. LF1
FIG|1318.8.peg.1495; Streptococcus parasanguinis
FIG|1381091.3.peg.1173; Streptococcus equi subsp. zooepidemicus SzAM60
FIG|1409369.3.peg.815; Staphylococcus aureus AMMC6050
FIG|1497681.5.peg.3014; Listeria newyorkensis
FIG|1095727.3.peg.409; Streptococcus sp. SK643
FIG|1318.12.peg.313; Streptococcus parasanguinis
FIG|1681184.3.peg.4899; Lysinibacillus sp. ZYM-1
FIG|561879.29.peg.3569; Bacillus safensis
FIG|1884359.3.peg.2978; Psychrobacillus sp. OK028
FIG|29367.3.peg.1112; Clostridium puniceum
FIG|225345.3.peg.1103; Clostridium chromiireducens
FIG|1345695.10.peg.2438; Clostridium saccharobutylicum DSM 13864
FIG|119641.3.peg.784; Clostridium uliginosum
FIG|1761781.3.peg.88; Clostridium sp. DSM 8431
FIG|1946346.3.peg.2999; Clostridium sp. UBA1056
FIG|1492.48.peg.3952; Clostridium butyricum
FIG|1121302.3.peg.4511; Clostridium cavendishii DSM 21758
FIG|398512.4.peg.5301; Pseudobacteroides cellulosolvens ATCC 35603 = DSM 2933
FIG|1946357.3.peg.500; Clostridium sp. UBA3947
FIG|642492.3.peg.3338; Cellulosilyticum lentocellum DSM 5427
FIG|1946690.3.peg.1553; Lachnoclostridium sp. UBA3320
FIG|397290.3.peg.150; Lachnospiraceae bacterium A2
FIG|97138.3.peg.1713; Clostridium sp. ASF356
FIG|1965545.3.peg.499; Tyzzerella sp. An114
FIG|1047063.3.peg.240; WS1 bacterium JGI 0000059-K21
FIG|1192034.3.peg.1508; Chondromyces apiculatus DSM 436
AAA88323.1; Myxococcus xanthus Retron-Mxa2 (Mx65)
FIG|33.8.peg.8196; Myxococcus fulvus
FIG|215803.3.peg.1485; Enhygromyxa salina
FIG|1406225.3.peg.2150; Archangium violaceum Cb vi76
FIG|1952931.3.peg.5137; Verrucomicrobia subdivision 3 bacterium UBA6082
FIG|1972460.3.peg.279; Anaerolineaceae bacterium 4572_78
FIG|1950201.3.peg.3297; Anaerolineales bacterium UBA2796
WP_015247705.1;
FIG|1799658.3.peg.2777; Planctomycetaceae bacterium SCGC AG-212-F19
FIG|214688.26.peg.6659; Gemmata obscuriglobus UQM 2246
FIG|2023130.3.peg.4250; Rhodopirellula sp. MGV
FIG|52.7.peg.9046; Chondromyces crocatus
FIG|448385.16.peg.2914; Sorangium cellulosum So ce56
FIG|888845.4.peg.14202; Minicystis rosea
FIG|1752210.3.peg.1275; Deltaproteobacteria bacterium Ga0077539
FIG|1391654.3.peg.3562; Labilithrix luteola
FIG|1752218.3.peg.3670; Planctomycetaceae bacterium Ga0077529
WP_006981058.1; Chthoniobacter flavus
FIG|1952939.3.peg.2584; Verrucomicrobiaceae bacterium UBA1938
FIG|2024858.3.peg.3711; Sandaracinus sp.
WP_009096166.1; Rhodopirellula sp. SWK7
FIG|595453.3.peg.1506; Rhodopirellula sp. SM50
FIG|1263868.3.peg.4100; Rhodopirellula europaea SH398
FIG|167547.3.peg.303; Prochlorococcus marinus str. MIT 9311
FIG|1499501.3.peg.459; Prochlorococcus sp. SS52
FIG|1905359.3.peg.4335; marine bacterium AO1-C
WP_002700020.1; Microscilla marina
FIG|1913989.145.peg.263; Gammaproteobacteria bacterium
WP_073154989.1; Seinonella peptonophila
FIG|46223.3.peg.3648; Thermoflavimicrobium dichotomicum
FIG|1329796.3.peg.1947; Risungbinella massiliensis
FIG|1123252.3.peg.3225; Shimazuella kribbensis DSM 45090
FIG|714067.3.peg.3719; Kroppenstedtia eburnea
FIG|1341151.3.peg.628; Laceyella sacchari 1-1
FIG|2026763.3.peg.3089; Myxococcales bacterium
FIG|1797895.3.peg.2518; Deltaproteobacteria bacterium RIFOXYA12_FULL_58_15
FIG|373672.4.peg.4082; Chryseobacterium gambrini
FIG|1416778.5.peg.4374; Chryseobacterium arachidis
FIG|1603293.4.peg.829; Flavobacterium sp. 316
CCB70859.1; Flavobacterium branchiophilum FL-15
FIG|1986952.3.peg.951; Sphingobacteriaceae bacterium GW460-11-11-14-LB5
FIG|1476464.3.peg.4304; Pedobacter xixiisoli
FIG|1761785.3.peg.3112; Flavobacterium sp. ov086
FIG|1664068.3.peg.3690; bacterium 336/3
FIG|880071.3.peg.3500; Bernardetia litoralis DSM 6794
FIG|1121902.3.peg.2906; Eisenibacter elegans DSM 3317
FIG|1509483.4.peg.1923; Flectobacillus sp. BAB-3569
FIG|1166018.3.peg.5312; Fibrella aestuarina BUZ 2
FIG|634771.3.peg.967; Chitinophaga eiseniae
FIG|29529.3.peg.396; Chitinophaga arvensicola
FIG|1891659.3.peg.6032; Chitinophaga sp. CB10
FIG|2033437.3.peg.3442; Chitinophaga sp. MD30
FIG|1004.4.peg.1677; Chitinophaga sancti
FIG|1881041.3.peg.3698; Chitinophaga sp. YR627
FIG|1123078.3.peg.2010; Runella zeae DSM 19591
FIG|354355.3.peg.1846; Niastella yeongjuensis
FIG|1951546.3.peg.1574; Chitinophagaceae bacterium UBA1946
FIG|1812911.3.peg.633; Flavihumibacter sp. CACIAM 22H1
FIG|477680.4.peg.4668; Filimonas lacunae
FIG|221126.7.peg.3781; Algibacter lectus
FIG|342954.4.peg.734; Lacinutrix algicola
FIG|1871037.5.peg.2180; Flavobacteriaceae bacterium
FIG|669041.3.peg.843; Tenacibaculum dicentrarchi
WP_074538568.1; Cellulophaga baltica
FIG|1248440.3.peg.1511; Polaribacter franzmannii ATCC 700399
FIG|1121007.3.peg.1574; Aquimarina muelleri DSM 19832
FIG|688867.3.peg.2332; Ohtaekwangia koreensis
FIG|926565.3.peg.717; Sporocytophaga myxococcoides DSM 11118
FIG|1257021.3.peg.5265; Flammeovirgaceae bacterium 311
FIG|2044937.5.peg.2350; candidate division KSB3 bacterium
FIG|1499966.3.peg.180; Candidatus Moduliflexus flocculans
FIG|1948269.3.peg.1254; Verrucomicrobia bacterium UBA6053
FIG|694433.3.peg.3103; Saprospira grandis DSM 2844
FIG|2008677.3.peg.3337; Mitsuaria noduli
FIG|946333.3.peg.3570; Rhizobacter gummiphilus
FIG|1736433.3.peg.5559; Rhizobacter sp. Root1221
FIG|1500265.3.peg.5760; Methylibium sp. YR605
FIG|1121349.4.peg.2836; Comamonas composti DSM 21721
FIG|1082851.3.peg.91; Comamonas serinivorans
FIG|1121480.5.peg.5468; Pseudoduganella violaceinigra DSM 15887
FIG|2045208.3.peg.1247; Massilia violaceinigra
FIG|1736455.3.peg.3692; Massilia sp. Root133
FIG|34073.25.peg.8569; Variovorax paradoxus
FIG|1884311.3.peg.7121; Variovorax sp. OK202
FIG|1123487.3.peg.1763; Uliginosibacterium gangwonense DSM 18521
FIG|2029111.3.peg.3032; Comamonadaceae bacterium NML120219
FIG|1977087.20.peg.1226; Proteobacteria bacterium
FIG|754436.4.peg.4454; Photobacterium aphoticum
FIG|265726.7.peg.1038; Photobacterium halotolerans
FIG|1121867.3.peg.59; Enterovibrio calviensis DSM 14347
FIG|1238431.3.peg.2655; Vibrio nigripulchritudo BLFn1
FIG|1384589.3.peg.2721; [Erwinia] teleogrylli
FIG|1261127.3.peg.2947; Citrobacter amalonaticus Y19
FIG|349521.8.peg.3588; Hahella chejuensis KCTC 2396
FIG|525918.3.peg.1501; Thiothrix caldifontis
FIG|1737490.4.peg.4974; Agarilytica rhodophyticola
FIG|251229.3.peg.427; Chroococcidiopsis thermalis PCC 7203
FIG|1245923.3.peg.9587; Scytonema millei VB511283
FIG|1503470.5.peg.10896; cyanobacterium TDX16
FIG|2005460.3.peg.1118; Chondrocystis sp. NIES-4102
FIG|179408.3.peg.4679; Oscillatoria nigro-viridis PCC 7112
FIG|1612423.3.peg.5384; Nostoc linckia z1
FIG|63737.11.peg.472; Nostoc punctiforme PCC 73102
FIG|224013.5.peg.7163; Nostoc piscinale CENA21
FIG|1932621.3.peg.7363; Nostoc sp. T09
FIG|373994.3.peg.3383; Rivularia sp. PCC 7116
FIG|2005463.3.peg.257; Calothrix sp. NIES-4105
FIG|2005459.3.peg.7019; Tolypothrix sp. NIES-4075
FIG|184925.3.peg.2602; Chlorogloeopsis fritschii PCC 9212
FIG|454136.5.peg.3127; Phormidium ambiguum IAM M-71
FIG|203124.6.peg.2732; Trichodesmium erythraeum IMS101
FIG|2040638.3.peg.3067; Tychonema bourrellyi FEM_GT703
FIG|1880991.4.peg.2927; Oscillatoriales cyanobacterium USR001
FIG|1173028.3.peg.7115; Oscillatoria sp. PCC 10802
FIG|568701.4.peg.2073; Moorea bouillonii PNG
FIG|927677.3.peg.4187; Synechocystis sp. PCC 7509
FIG|179408.3.peg.6267; Oscillatoria nigro-viridis PCC 7112
FIG|1710894.3.peg.2079; Aphanizomenon flos-aquae LD13
FIG|1947888.3.peg.4484; Cyanobacteria bacterium UBA6047
FIG|1705388.3.peg.1178; Planktothricoides sp. SR001
FIG|454136.5.peg.4162; Phormidium ambiguum IAM M-71
FIG|2005458.3.peg.378; Nostoc sp. NIES-4103
FIG|1947874.3.peg.4590; Cyanobacteria bacterium UBA1583
FIG|1781255.3.peg.802; Desertifilum sp. IPPAS B-1220
FIG|1128427.4.peg.2346; filamentous cyanobacterium ESFC-1
FIG|1946321.3.peg.3928; Chloracidobacterium sp. UBA7656
FIG|118173.3.peg.1030; Pseudanabaena sp. PCC 6802
FIG|1922337.4.peg.4802; Leptolyngbya sp. ‘hensonii’
FIG|927668.3.peg.2766; Pseudanabaena biceps PCC 7429
FIG|1173020.3.peg.6191; Chamaesiphon minutus PCC 6605
FIG|329726.14.peg.4440; Acaryochloris marina MBIC11017
FIG|215803.3.peg.1649; Enhygromyxa salina
FIG|1920190.3.peg.9548; Archangium sp. Cb G35
FIG|1961464.3.peg.5181; Myxococcales bacterium UBA2376
FIG|765913.3.peg.336; Thiorhodococcus drewsii AZ1
FIG|1396141.3.peg.2891; Haloferula sp. BvORR071
FIG|1961463.3.peg.5253; Myxococcales bacterium UBA1671
AAL40743.1; Nannocystis exedens Retron-Nex2 (Ne144)
FIG|54.3.peg.4123; Nannocystis exedens
FIG|53367.3.peg.3417; Asanoa ferruginea
FIG|460265.11.peg.3882; Methylobacterium nodulans ORS 2060
FIG|298794.3.peg.462; Methylobacterium variabile
FIG|190148.4.peg.3492; Bradyrhizobium paxllaeri
FIG|1075417.3.peg.445; Catalinimonas alkaloidigena
FIG|1429438.4.peg.7505; Candidatus Entotheonella factor
FIG|1977087.12.peg.1756; Proteobacteria bacterium
FIG|92487.3.peg.3972; Thiothrix eikelboomii
FIG|1977087.20.peg.1473; Proteobacteria bacterium
FIG|1123400.3.peg.3276; Thiofilum flexile DSM 14609
FIG|34062.8.peg.73; Moraxella osloensis
FIG|1699623.3.peg.1502; Psychrobacter sp. P11G3
FIG|1123509.3.peg.848; Zooshikella ganghwensis DSM 15267
FIG|2026735.3.peg.2222; Deltaproteobacteria bacterium
FIG|1977087.12.peg.2982; Proteobacteria bacterium
FIG|2026763.4.peg.1195; Myxococcales bacterium
FIG|1977087.12.peg.510; Proteobacteria bacterium
FIG|1123508.3.peg.7252; Zavarzinella formosa DSM 19928
FIG|214688.26.peg.3091; Gemmata obscuriglobus UQM 2246
FIG|1908690.5.peg.1204; Fimbriiglobus ruber
FIG|1805126.3.peg.4431; Deltaproteobacteria bacterium CG2_30_63_29
FIG|1882752.4.peg.1962; Singulisphaera sp. GP187
FIG|1636152.3.peg.5364; Planctomyces sp. SH-PL62
APR75442.1; Minicystis rosea
FIG|54.3.peg.8798; Nannocystis exedens
FIG|980254.4.peg.4083; Roseimaritima ulvae
FIG|1856297.3.peg.3627; Gammaproteobacteria bacterium 45_16_T64
FIG|1219077.3.peg.1945; Vibrio azureus NBRC 104587
FIG|1334629.3.peg.167; Myxococcus fulvus 124B02
AAA25405.1; Myxococcus xanthus Retron-Mxal (Mx162)
FIG|378806.16.peg.4444; Stigmatella aurantiaca DW4/3-1
WP_002615305.1; Stigmatella aurantiaca Retron-Sau1 (Sa163)
FIG|48.3.peg.757; Archangium gephyra
FIG|448385.16.peg.2083; Sorangium cellulosum So ce56
FIG|52.7.peg.5100; Chondromyces crocatus
FIG|1752210.3.peg.5621; Deltaproteobacteria bacterium Ga0077539
FIG|2024858.3.peg.6345; Sandaracinus sp.
WP_012826728.1; Haliangium ochraceum
FIG|927083.3.peg.3408; Sandaracinus amylolyticus
WP_006977315.1; Plesiocystis pacifica
FIG|1400863.5.peg.627; Candidatus Competibacter denitrificans_Run A_D11
FIG|1961463.3.peg.4303; Myxococcales bacterium UBA1671
FIG|1898731.3.peg.3099; Curtobacterium sp. MCBA15_001
FIG|1279028.3.peg.3374; Curtobacterium sp. 314Chir4.1
FIG|1898733.3.peg.2056; Curtobacterium sp. MCBA15_004
FIG|1795630.3.peg.3476; Frondihabitans sp. PAMC 28766
FIG|2033654.3.peg.3461; Curtobacterium sp. ‘Ferrero’
FIG|1736329.5.peg.1436; Frondihabitans sp. Leaf304
FIG|1736292.3.peg.1083; Rathayibacter sp. Leaf185
FIG|1736327.3.peg.206; Rathayibacter sp. Leaf296
FIG|1736311.3.peg.3668; Curtobacterium sp. Leaf261
FIG|1736308.3.peg.2333; Frigoribacterium sp. Leaf254
FIG|656366.8.peg.2905; Arthrobacter alpinus
FIG|494023.3.peg.138; Paeniglutamicibacter antarcticus
ASN40093.1; Arthrobacter sp. 7749
FIG|1494608.3.peg.465; Arthrobacter sp. PAMC 25486
FIG|656366.4.peg.2620; Arthrobacter alpinus
FIG|656366.3.peg.1944; Arthrobacter alpinus
FIG|1132441.3.peg.1888; Arthrobacter sp. 35W
FIG|1704044.3.peg.520; Arthrobacter sp. ERGS1:01
FIG|1496689.3.peg.681; Arthrobacter sp. L77
FIG|1681197.3.peg.149; Arthrobacter sp. RIT-PI-e
FIG|37921.12.peg.1481; Arthrobacter agilis
FIG|1736303.3.peg.982; Arthrobacter sp. Leaf234
FIG|1312978.3.peg.1472; Arthrobacter sp. H41
FIG|1348338.3.peg.1472; Leifsonia rubra CMS 76R
FIG|1452536.3.peg.1955; Microbacterium sp. Cr-K20
FIG|1736525.3.peg.446; Leifsonia sp. Root4
FIG|1529318.3.peg.434; Cryobacterium sp. MLB-32
FIG|1267973.3.peg.3479; Arthrobacter sp. H5
FIG|150121.3.peg.1900; Agreia pratensis
FIG|123316.3.peg.955; Agreia sp. VKM Ac-2052
FIG|1052260.3.peg.3617; Klenkia soli
FIG|1566299.3.peg.3962; Klenkia marina
FIG|1736356.3.peg.3150; Modestobacter sp. Leaf380
FIG|1736354.3.peg.1787; Geodermatophilus sp. Leaf369
FIG|479431.6.peg.3115; Nakamurella multipartita DSM 44233
FIG|1090615.3.peg.2397; Nakamurella panacisegetis
FIG|1306174.4.peg.4778; Kineosporia aurantiaca JCM 3230
FIG|546871.3.peg.1120; Friedmanniella luteola
FIG|630515.4.peg.525; Microlunatus soli
FIG|546874.3.peg.1181; Friedmanniella sagamiharensis
BAK35674.1; Microlunatus phosphovorus NM-1
FIG|1380390.4.peg.72; Solirubrobacterales bacterium URHD0059
FIG|1283299.3.peg.2784; Conexibacter woesei Iso977N
FIG|929712.3.peg.3165; Patulibacter minatonensis DSM 18081
FIG|1123262.3.peg.3125; Solirubrobacter soli DSM 22325
FIG|1861.4.peg.5240; Geodermatophilus obscurus
FIG|1137993.4.peg.1318; Geodermatophilus africanus
FIG|1070870.3.peg.778; Geodermatophilus nigrescens
FIG|1190417.3.peg.1785; Geodermatophilus telluris
FIG|477641.3.peg.1023; Modestobacter marinus
FIG|1798228.3.peg.3756; Blastococcus sp. DSM 46838
WP_091929708.1; Blastococcus sp. DSM 46786
SHH20361.1; Jatrophihabitans endophyticus
FIG|1844.3.peg.1151; Nocardioides luteus
FIG|748909.6.peg.1418; Nocardioides alpinus
FIG|402596.3.peg.987; Nocardioides exalbidus
FIG|1736322.3.peg.1963; Nocardioides sp. Leaf285
FIG|1445613.3.peg.3490; Sciscionella sp. SE31
FIG|543632.4.peg.9742; Actinoplanes subtropicus
FIG|1036182.3.peg.2958; Actinoplanes atraurantiacus
FIG|1246995.3.peg.737; Actinoplanes friuliensis DSM 7358
FIG|56427.3.peg.3052; Couchioplanes caeruleus subsp. caeruleus
FIG|1710355.3.peg.2225; Actinoplanes sp. TFC3
FIG|649831.3.peg.2352; Actinoplanes sp. N902-109
FIG|35754.4.peg.6321; Dactylosporangium aurantiacum
FIG|1881.4.peg.2703; Micromonospora viridifaciens
FIG|47863.3.peg.3975; Micromonospora globosa
FIG|285665.4.peg.2050; Micromonospora coriariae
FIG|1192034.3.peg.4668; Chondromyces apiculatus DSM 436
FIG|1198133.3.peg.2442; Myxococcus xanthus DZ2
FIG|33.8.peg.521; Myxococcus fulvus
FIG|394193.3.peg.7794; Amycolatopsis saalfeldensis
FIG|369932.4.peg.5621; Amycolatopsis niigatensis
FIG|1238180.3.peg.5340; Amycolatopsis azurea DSM 43854
FIG|589385.3.peg.7940; Amycolatopsis xylanica
FIG|1068980.3.peg.1527; Amycolatopsis nigrescens CSC17Ta-90
FIG|1854586.3.peg.2100; Amycolatopsis antarctica
FIG|587909.3.peg.3086; Yuhushiella deserti
FIG|2030.3.peg.3051; Kibdelosporangium aridum
FIG|1382595.4.peg.3164; Saccharopolyspora erythraea D
WP_013675061.1; Pseudonocardia dioxanivorans
FIG|1660131.3.peg.2805; Pseudonocardia sp. SCN 72-86
FIG|366584.3.peg.4349; Pseudonocardia oroxyli
FIG|1885031.4.peg.5241; Pseudonocardia sp. Ae331_Ps2
FIG|1690815.5.peg.5350; Pseudonocardia sp. HH130630-07
FIG|1123023.3.peg.3229; Pseudonocardia acaciae DSM 45401
FIG|1449976.3.peg.8114; Kutzneria albida DSM 43870
WP_007238159.1;
FIG|1220583.3.peg.1582; Gordonia aichiensis NBRC 108223
GAB07179.1; Gordonia amarae NBRC 15530
FIG|1223545.3.peg.704; Gordonia soli NBRC 108243
FIG|1223540.3.peg.3237; Gordonia desulfuricans NBRC 100010
FIG|1112204.3.peg.4913; Gordonia polyisoprenivorans VH2
AFR49048.1; Gordonia sp. KTR9
FIG|402289.3.peg.1558; Rhodococcus sp. HA99
FIG|1077144.3.peg.224; Dietzia alimentaria 72
FIG|1344003.3.peg.1864; Williamsia sterculiae
FIG|1463823.3.peg.3407; Microbispora sp. NRRL B-24597
FIG|1903117.3.peg.1566; Williamsia sp. 1138
FIG|1603258.4.peg.1828; Williamsia herbipolensis
FIG|644548.3.peg.652; Gordonia neofelifaecis NRRL B-59395
FIG|1136941.3.peg.1310; Gordonia phthalatica
FIG|1223542.3.peg.3439; Gordonia malaquae NBRC 108250
FIG|473 12.10.peg.4232; Tsukamurella pulmonis
FIG|57704.14.peg.421; Tsukamurella tyrosinosolvens
FIG|521096.6.peg.2443; Tsukamurella paurometabola DSM 20162
FIG|1123241.3.peg.3642; Nakamurella lactea DSM 19367
FIG|1210073.4.peg.1031; Nocardia salmonicida NBRC 100378
FIG|1206740.4.peg.4617; Nocardia thailandica NBRC 100428
FIG|1210064.4.peg.2434; Nocardia altamirensis NBRC 108246
FIG|1123258.3.peg.1651; Smaragdicoccus niigatensis DSM 44881 = NBRC 103563
FIG|1443888.3.peg.2891; Rhodococcus fascians 02-815
FIG|1517936.4.peg.882; Rhodococcus sp. CUA-806
FIG|398843.6.peg.4214; Rhodococcus kyotonensis
FIG|1813677.3.peg.4031; Rhodococcus sp. EPR-157
WP_008711873.1; organism not named
FIG|1381122.3.peg.6103; Rhodococcus erythropolis DN1
FIG|1736210.3.peg.2766; Rhodococcus sp. Leaf7
FIG|1736300.3.peg.1279; Rhodococcus sp. Leaf225
FIG|1219012.3.peg.1705; Rhodococcus corynebacterioides NBRC 14404
FIG|1219023.3.peg.2791; Rhodococcus rhodnii NBRC 100604
FIG|616997.3.peg.2548; Hoyosella altamirensis
FIG|1303689.4.peg.2934; Rhodococcus koreensis JCM 10743 = NBRC 100607
FIG|644.85.peg.4392; Aeromonas hydrophila.

Use of Sequence Listing to Identify Cognate RT ncRNA Pairs

A cognate pairing of a ncRNA and an RT (amino acid or nucleotide sequence) can easily be identified from information presented/included in the sequence listing by identifying those ncRNA sequences from Table B and those RT sequences from Table A have the same genomic accession number that is indicated for each sequence in the Sequence Listing. In various other aspects, the retron RT component and the ncRNA component can be from different bacterial species, i.e., are not found together in nature as cognate pairs. In still other embodiments, the retron RT component and the ncRNA component can both be from the same phylogenetic functional type (e.g., Type I-A, Type I-B1, Type I-B2, Type IC, etc.). For example, a recombinant retron-based genome editing system may be comprised of a retron RT from Type I-A (i.e., SEQ ID Nos: 3980-4178 for AA and SEQ ID Nos: 11231-11429 for NT-see Table A) and a retron ncRNA also from Type I-A (i.e., SEQ ID Nos: 16886-17078—see Table B).

The sequence listing that is/was co-filed with this PCT Application forms a part of this Specification as originally filed. As noted above, the sequence listing may be consulted to identify a cognate pairing between a retron RT (of Table A-AA or NT) and a retron ncRNA (of Table B). The following steps may be followed:
Step 1. Pick an RT sequence. Select any RT sequence from Table A by its sequence identifier (e.g., SEQ ID NO: 4178).
Step 2. Locate the sequence entry in the sequence listing. Identify the selected sequence identifier (e.g., SEQ ID NO: 4178) in the sequence listing using any text-find function in a suitable text editing software used to read the sequence listing file (e.g., Microsoft Word) that was submitted with this Specification at the time of filing and which forms a part of this Specification as originally filed. Taking into account that the sequence listing as originally included in this Specification is in the format of an XML file, the search would be for the following text (in {brackets}): {sequenceIDNumber-“4178” }.
Step 3. Determine which organism and/or NCBI sequence reference number is associated with the sequence entry. Once Step 2 locates the searched text, inspect that sequence entry for the several text lines referring to “</INSDQualifier_value>” and note the NCBI reference sequence number displayed to the left of one of the identified lines. The designator “</INSDQualifier_value>” also indicates the source organism as well. For example, for entry {sequenceIDNumber-“4178” }, the organism identified at a first “</INSDQualifier_value>” is “Shewanell sp.” and the NCBI reference number identified at a second “</INSDQualifier_value>” is “Related to NZ_JWGX01000025.1” Thus the NCBI reference number is NZ_JWGX01000025.1 and the organism is Shewanella sp. for SEQ ID NO: 4178.
Step 4. Find the coganate retron ncRNA sequence. Conduct a text search of the sequence listing for the NCBI reference number identified in Step 3. Inspect the entry for the associated SEQ ID NO:. In the case of SEQ ID NO: 4178, the NCBI reference number is “NZ_JWGX01000025.1” and the associated ncRNA sequence is SEQ ID NO: 17078.

This further exemplified as follows:
Step 1. Pick any RT sequence from Table A (e.g., SEQ ID NO: 4178).
Step 2. Search and identify SEQ ID NO: 4178 in the sequence listing file by searching for the text in brackets as follows: {sequenceIDNumber=“4178” }. Inspect the hit region, which is imaged as the following computer screen image and which shows in bold the (a) amino acid sequence of SEQ ID NO: 4178, (b) the associated organism as “Shewanella sp.”, and (c) the associate NCBI reference number as “NZ_JWGX01000025.1”.

```
                              <INSDSeq_sequence>
MKKLLDYLTSSDNLKNYLKLTYPQISYDDDVFLINGKYHLSSLIKSAPKMYKFYTIPKRNGGRR
AIAKPSKKLKVTQRDLSTYLEKILPVADCAFAYRANKSIKDNALIHVGNKELLKMDLYNFENSI
TPKMLINEIKESDLSFEESELDYIKKLAFWCPSKEDNGKLILSVGAPSSPLLSNFIMYRFDSVL
SEVAKSNKVKYSRYADDIFLSINVPNVLKLFVNIVKIILKEMIKDELIINEMKIVESSKAHNRH
VIGITITNDNKISIGRQNKRMISSLVHKYSLGYLDTSKIIRLRGLISFASSIEPDFIIRLENKY
TENLINKLIKGLD</INSDSeq_sequence>
                        <INSDSeq>
                        <INSDSeq_length>339</INSDSeq_length>
                        <INSDSeq_moltype>AA</INSDSeq_moltype>
                        <INSDSeq_division>PAT</INSDSeq_division>
                        <INSDSeq_feature-table>
                            <INSDFeature>
                                <INSEFeature_key>
source</INSDFeature_key>
            <INSDFeature_location>1..339</INSDFeature_location>
                                <INSDFeature_quals>
                                    <INSDQualifier>
                                        <INSDQualifier_name>
mol_type</INSDQualifier_name>
                                        <INSDQualifier_value>other
protein</INSDQualifier_value>
                                      </INSDQualifier>
                                      <INSDQualifier>
                                          <INSDQualifier_name>
organism</INSDQualifier_name>
                                          <INSDQualifier_value>
Shewanella sp.</INSDQualifier_value>
                                      </INSDQualifier>
                                      <INSDQualifier>
                                          <INSDQualifier_name>
note</INSDQualifier_name>
                                        <INSDQualifier_value>
Related to NZ_JWGX01000025.1</INSDQualifier_value>
                                    </INSDQualifier>
                                </INSDFeature_quals>
                            </INSDFeature>
                        </INSDSeq_feature-table>
                        <INSDSeq_sequence>
HEELQKSLLASNEVKEEFSQKASHIFSESGQKLPNAAESLALAALQNVKYQGFYMYKVYTIPKE
```

Step 3. Identify the NCBJ reference number. In this example, the NCBI reference number is NZ_JWGX01000025.1.

Step 4. The NCBJ reference number is then used to locate the cognate retron ncRNA sequence in the sequence listing. In this case, the cognate ncRNA sequence is SEQ ID NO: 17078, as shown in the below computer screen image of the ncRNA sequence entry for SEQ ID NO: 17078, which as can be seen by inspection, shares the NCBJ reference number of “NZ_JWGX010000025.1” and the organism of “Shewanella sp.”

```
                          <INSDSeq_sequence>
actctttagcgttcgggttaaatattaatttacctagtcgggcgattagccagactgaaactta
ttgaacgggtttttgggtgcgaaagcatcacatcctaaattcgttcctcatttaggatgtgatg
ctttcgcttcctctagtaaagggtt</INSDSeq_sequence>
                        </INSDSeq>
        </sequenceData>
        <SequenceData sequenceIDNumber=“17078”>
                        <INSDSeq>
                        <INSDSeq_length>190</INSDSeq_length>
                        <INSDSeq_moltype>RNA</INSDSeq_moltype>
                        <INSDSeq_division>PAT</INSDSeq_division>
                        <INSDSeq_feature-table>
                            <INSDFeature>
                                <INSDFeature_key>
source</INSDFeature_key>
            <INSDFeature_location>1..190</INSDFeature_location>
                                <INSDFeature_quals>
                                    <INSDQualifier>
                                        <INSDQualifier_name>
mol_type</INSDQualifier_name>
                                        <INSDQualifier_value>other
RNA</INSDQualifier_value>
                                      </INSDQualifier>
                                      <INSDQualifier>
                                          <INSDQualifier_name>
organism</INSDQualifier_name>
                                          <INSDQualifier_value>
Shewanella sp.</INSDQualifier_value>
                                      </INSDQualifier>
                                      <INSDQualifier>
                                          <INSDQualifier_name>
note</INSDQualifier_name>
                                        <INSDQualifier_value>
Related to NZ_JWGX01000025.1</INSDQualifier_value>
                                    </INSDQualifier>
                                </INSDFeature_quals>
                            </INSDFeature>
                        </INSDSeq_feature-table>
                        <INSDSeq_sequence>
gctgtttagcgttttggggttttaaggtgttcaatgatcaatgtaatcagcctagtcgggtgta
```

Thus, the cognate retron RT from Table A and retron ncRNA from Table B may be easily identified for embodiments involving a retron editing system that comprises a retron RT paired with its cognate retron ncRNA.

### EXEMPLARY EMBODIMENTS

Exemplary embodiment Group A

The following paragraphs describe exemplary and non-limiting embodiments of the present disclosure.

Paragraph 1. An engineered retron, comprising:
a) an msr locus (that encodes the msr RNA portion of an msDNA);
b) an msd locus encoding the msd RNA portion of the msDNA;
c) a retron reverse transcriptase (RT), wherein said msd RNA is capable of being reverse transcribed to form the msDNA by the retron reverse transcriptase (RT);
d) a heterologous nucleic acid inserted at or within the msd locus, upstream of the msr locus, upstream or downstream of the msd locus; and,
e) a sequence modification (e.g., insertion, deletion, and/or substitution of one or more nucleotide(s)) in the msr locus and/or the msd locus that:
   i) modulates (e.g., enhances) reverse transcription, processivity, accuracy/fidelity, and/or production of the msDNA (e.g., in the mammalian cell);
   ii) modulates (e.g., reduces) immunogenicity of ncRNA encoded by the engineered retron (e.g., the msr locus and/or the msd locus) in a host (e.g., a host comprising the mammalian cell); and/or
   iii) comprises a nucleotide sequence that modulates (e.g., inhibits, either permanently or transiently) a function of the msDNA; and/or
   iv) modulates (e.g., improves) efficiency of targeted genome editing/engineering.

Paragraph 2. The engineered retron of paragraph 1, wherein said engineered retron is engineered based on and/or to resemble a secondary structure of a wild-type or consensus retron encoding a wild-type or consensus retron ncRNA encompassed by:
1) any one of the sequences and/or structures as depicted in SEQ ID NOs: 1-21 and/or FIGS. 2-22; or
2) a variant of 1), having:
   A) up to 1, 2, or 3 (e.g., up to 1) nucleotide changes per 10 red lettered-nucleotides;
   B) up to 4, 5, or 6 (e.g., up to 1 or 2) nucleotide changes per 10 black lettered-nucleotides; and/or
   C) up to 7, 8, or 9 (e.g., up to 3 or 4) nucleotide changes per 10 grey lettered-nucleotides; and/or
optionally further comprising:
   a) 7, 8, 9, or 10 (e.g., 9 or 10) nucleotides present per 10 red-circled nucleotides;
   b) 6, 7, 8, 9, or 10 (e.g., 8, 9 or 10) nucleotides present per 10 black-circled nucleotides;
   c) 4, 5, 6, 7, 8, 9, or 10 (e.g., 6, 7, 8, 9 or 10) nucleotides present per 10 grey-circled nucleotides; and/or
   d) 2, 3, 4, 5, 6, 7, 8, 9, or 10 (e.g., 4, 5, 6, 7, 8, 9, or 10) nucleotides present per 10 white-circled nucleotides.

Paragraph 3. The engineered retron of any one of paragraphs 1-2, wherein said engineered retron is engineered by introducing said sequence modification into a wild-type retron encoding a wild-type ncRNA, or into a synthetic/artificial retron encoding the consensus retron ncRNA.

Paragraph 4. The engineered retron of any one of paragraphs 1-3, wherein the sequence modification comprises, in the encoded retron ncRNA, one or more of:
(i) a modified (e.g., mutated, reduced, or eliminated) bulge in a1, a2 or both a1 and a2;
(ii) an extension or shortening of a1, a2 or both a1 and a2;
(iii) an extension or shortening of a spacer sequence between hairpin loops (e.g., S1, S2, S3, S4, S5 and/or S6);
(iv) a modified (e.g., mutated or eliminated) bulge in hairpin loops (e.g., L1, L2, L3, L4, L5 and/or L6 (e.g., by removing unpaired bases in the bulge, or by replacing unpaired bases with an equivalent number of base pairs));
(v) a modified (e.g., extended or shortened) length of hairpin loops (e.g., L1, L2, L3, L4, L5 and/or L6);
(vi) an alternative L1 and/or L2 having complement, reverse, or reverse complement sequences;
(vii) a modified (e.g., increased) number of unpaired bases at the tip of hairpin loops (e.g., L1, L2, L3, L4, L5 and/or L6);
(viii) a modified (e.g., increased or decreased) GC content in hairpin loops (e.g., L1, L2, L3, L4, L5 and/or L6);
(ix) an insertion of the heterologous nucleic acid in spacer sequences between hairpin loops (e.g., S1, S2, S3, S4, S5 and/or S6), or at the tip of hairpin loops (e.g., L1, L2, L3, L4, L5 and/or L6);
(x) a deletion of hairpin loops (e.g., L1, L2, L3, L4, L5 and/or L6);
(xi) an addition of a new loop in spacer sequences between hairpin loops (e.g., S1, S2, S3, S4, S5 and/or S6);
(xii) circularization of the ncRNA with the 5′ end and the 3′ end of the ncRNA being connected either directly, or via a spacer sequence;
(xiii) a repositioned branching guanosine capable of initiating reverse transcription priming;
(xiv) a staggered end sequence that reduces immunogenicity of the retron ncRNA, created by, e.g., adding or removing the 5′ al nucleotides and/or the 3′ a2 nucleotides; and/or,
(xv) an antisense sequence complementary to a CRISPR/Cas guide RNA (gRNA) sequence encoded by the heterologous nucleic acid, wherein the antisense sequence hybridizes to and inhibits said gRNA in the encoded retron ncRNA, and wherein said antisense sequence is removed upon reverse transcription of the msDNA.

Paragraph 5. The engineered retron of any one of paragraphs 1-4, comprising:
(i) a heterologous nucleic acid (such as the coding sequence for an RNA aptamer or a ribozyme) inserted into the msr locus or the msd locus (such as in an S region (e.g., S1, S2, S3, S4, S5 and/or S6), or the tip of an L region (e.g., L1, L2, L3, L4, L5 and/or L6)), or upstream or downstream of either the msr locus or the msd locus; or,
(ii) a first heterologous nucleic acid inserted into the msd locus, and a second heterologous nucleic acid inserted either upstream of the msr locus or downstream of the msd locus, wherein the second heterologous nucleic acid encodes a CRISPR/Cas guide RNA (gRNA).

Paragraph 6. The engineered retron of any one of paragraphs 1-5, wherein said retron reverse transcriptase (RT) is a cognate RT, a retron RT from a species within the same clade of the cognate RT, or a retron RT not within the same clade of the cognate RT (such as an unrelated RT or an engineered RT).

Paragraph 7. The engineered retron of any one of paragraphs 1-6, wherein said heterologous nucleic acid encodes a protein or peptide of interest, or wherein said heterologous nucleic acid comprises or encodes a donor/template sequence (e.g., a donor that corrects/repairs/removes a mutation at the target genome site, such as a mutated exon in a disease gene; a functional DNA element (such as a promoter, an enhancer, a protein binding sequence, a methylation site, a homology region for assisting gene editing, etc.); or a coding sequence for a functional RNA element (ncRNAs, etc)).

Paragraph 8. The engineered retron paragraph 7, wherein said protein or peptide of interest comprises a therapeutic protein (such as a wt protein defective in a disease cell, or a therapeutic antibody or antigen-binding fragment thereof) useful in treating a disease.

Paragraph 9. The engineered retron of paragraph 7, wherein said donor/template sequence corrects/repairs/removes a mutation at the target genome site (such as a mutated exon in a disease gene).

Paragraph 10. The engineered retron of any one of paragraphs 1-9, further comprising or encoding a sequence-specific nuclease (such as a CRISPR/Cas effector enzyme, a ZFN, a TALEN, a meganuclease, TnpB, IscB, or a restriction endonuclease (RE)), and/or a DNA-repair modulating biomolecule.

Paragraph 11. The engineered retron of paragraph 10, wherein the sequence-specific nuclease is fused to the RT, optionally via a flexible linker (e.g., a flexible linker comprising Gly and Ser rich sequences such as G4S repeats or GS repeats) or by a generally disordered protein sequence (such as unstructured hydrophilic, biodegradable protein polymer, e.g., an XTEN peptide polymer).

Paragraph 12. The engineered retron of paragraph 10 or 11, wherein the nuclease is a CRISPR/Cas effector enzyme that forms a complex with a guide RNA (gRNA) recognizing a target sequence, wherein the gRNA is linked to the ncRNA and/or the msDNA, either directly or through a linker/spacer polynucleotide.

Paragraph 13. The engineered retron of paragraph 10, wherein the DNA-repair modulating biomolecule is a regulatory protein that modulates (e.g., enhances) HDR, and the regulatory protein is fused to the RT or to the sequence-specific nuclease, optionally via a flexible linker (e.g., the flexible linker comprising Gly and Ser rich sequences such as G4S repeats or GS repeats) or by a generally disordered protein sequence (such as unstructured hydrophilic, biodegradable protein polymer, e.g., an XTEN peptide polymer).

Paragraph 14. A vector system comprising a vector comprising the engineered retron of any one of paragraphs 1-13.

Paragraph 15. The vector system of paragraph 14, wherein the msr locus, the msd locus, and the RT gene are provided by the same vector.

Paragraph 16. The vector system of paragraph 14 or 15, wherein the same vector comprises a promoter operably linked to the msr locus and/or the msd locus.

Paragraph 17. The vector system of any one of paragraphs 14-16, wherein the promoter is further operably linked to the RT gene.

Paragraph 18. The vector system of paragraph 14, wherein the msr locus, the msd locus, and the RT gene are provided by at least two different vectors.

Paragraph 19. The vector system of paragraph 14 or 18, wherein said RT gene is provided in trans with respect to the msr gene and/or the msd gene.

Paragraph 20. The vector system of any one of paragraphs 14-19, wherein the one or more vectors comprise a viral vector and/or a nonviral vector.

Paragraph 21. The vector system of paragraph 20, wherein the nonviral vector comprises a plasmid.

Paragraph 22. The vector system of any one of paragraphs 14-21, comprising a vector encoding a sequence-specific nuclease.

Paragraph 23. The vector system of paragraph 22, wherein the sequence-specific nuclease comprises an RNA-guided sequence-specific nuclease (e.g., a CRISPR/Cas effector enzyme, an engineered RNA-guided FokI-nuclease (e.g., dCas-FokI), an RNA-guided DNA endonuclease, TnpB, IscB, or a transposon-associated nuclease), or a non-RNA-guided sequence-specific nuclease (e.g., a meganuclease, a zinc finger nuclease (ZFN), a TALE nuclease (TALEN), or a restriction endonuclease (RE)).

Paragraph 24. The vector system of paragraph 23, wherein the Cas effector enzyme is a Class 1, Type I, II, or III Cas; a Class 2, Type II Cas (e.g., Cas9); or a Class 2, Type V Cas (e.g., Cpf1).

Paragraph 25. The vector system of paragraph 23, wherein:
1) the RNA-guided sequence-specific nuclease comprises the CRISPR/Cas effector enzyme, the engineered RNA-guided FokI-nuclease (e.g., dCas-FokI), the RNA-guided DNA endonuclease, TnpB, IscB, IsrB, or the transposon-associated nuclease; or,
2) non-RNA-guided sequence-specific nuclease comprises the meganuclease, the zinc finger nuclease (ZFN), the TALE nuclease (TALEN), or the restriction endonuclease (RE).

Paragraph 26. The vector system of any one of paragraphs 14-25, further comprising a vector encoding a homologous recombination enhancer protein.

Paragraph 27. An isolated host cell comprising the engineered retron of any one of paragraphs 1-13, or the vector system of any of paragraphs 14-26.

Paragraph 28. The host cell of paragraph 27, wherein the host cell is a prokaryotic, archeon, or eukaryotic host cell.

Paragraph 29. The host cell of paragraph 28, wherein the eukaryotic host cell is a mammalian host cell.

Paragraph 30. The host cell of paragraph 28, wherein the eukaryotic host cell is a non-human host cell.

Paragraph 31. The host cell of paragraph 29, wherein the mammalian host cell is a human host cell.

Paragraph 32. The host cell of any of paragraphs 28-31, wherein the host cell is an artificial cell or genetically modified cell.

Paragraph 33. A pharmaceutical composition comprising the engineered retron of any one of paragraphs 1-13.

Paragraph 34. A delivery vehicle comprising the engineered retron of any one of paragraphs 1-13 or ncRNA encoded by the engineered retron of any one of paragraphs 1-13, the vector of any one of paragraphs 14-26, the host cell of any one of paragraphs 27-32, or the pharmaceutical composition of paragraph 33.

Paragraph 35. The delivery vehicle of paragraph 34, which is a lipid nanoparticle.

Paragraph 36. The delivery vehicle of paragraph 35, wherein the lipid nanoparticle comprises at least one cationic lipid selected from the group consists of: a lipid in Table (I), a lipid having a structure of Formula (I), a lipid having a structure of Formula (II), a lipid having a structure of Formula (III), a lipid having a structure of Formula (IV), a lipid having a structure of Formula (V), a lipid having a structure of Formula (VI), and combinations thereof.

Paragraph 37. A kit comprising the engineered retron of any one of paragraphs 1-13 or ncRNA encoded by the engineered retron of any one of paragraphs 1-13, the vector or vector system of any one of paragraphs 14-26, the host cell of any one of paragraphs 27-32, the pharmaceutical composition of paragraph 33, or the delivery vehicle of any one of paragraphs 34-36, and instructions for genetically modifying a cell with said engineered retron, ncRNA, vector/vector system, host cell, pharmaceutical composition, or delivery vehicle.

Paragraph 38. A method of modifying a target DNA sequence in a host (e.g., mammalian) cell, the method comprising introducing into the mammalian cell the engineered retron of any one of paragraphs 1-13 or ncRNA encoded by the engineered retron of any one of paragraphs 1-13, the vector or vector system of any one of paragraphs 14-26, the pharmaceutical composition of paragraph 33, or the delivery vehicle of any one of paragraphs 34-36, to allow the production of the msDNA in the host (e.g., mammalian) cell, wherein the encoded heterologous nucleic acid in the msDNA is integrated into the genome of the host (e.g., mammalian) cell at the target DNA sequence.

Paragraph 39. Use of the engineered retron of any one of paragraphs 1-13 or ncRNA encoded by the engineered retron of any one of paragraphs 1-13, the vector or vector system of any one of paragraphs 14-26, the pharmaceutical composition of paragraph 33, or the delivery vehicle of any one of paragraphs 34-36, to effect modification of a target DNA sequence in a host (e.g., mammalian) cell.

### Exemplary Embodiment Group B

The following paragraphs further describe exemplary and non-limiting embodiments of the present disclosure.

Paragraph 1. An engineered nucleic acid construct comprising:
a) a first polynucleotide encoding a non-coding RNA (ncRNA), said first polynucleotide comprising:
   1) an msr locus encoding the msr RNA portion of a multi-copy single-stranded DNA (msDNA); and
   2) an msd locus encoding the msd RNA portion of the msDNA; and
b) one or more heterologous nucleic acids inserted at or within a location selected from: the msd locus, upstream of the msr locus, upstream of the msd locus, and downstream of the msd locus,
wherein the ncRNA comprises:
(I) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity with an ncRNA listed in Table B; and/or
(II) an ncRNA having a conserved structure of any one of the ncRNA structures of FIGS. 2-27; and
wherein the ncRNA optionally excludes any ncRNA associated in nature with any one of the retron reverse transcriptases of Table X.

Paragraph 2. The engineered nucleic acid construct of paragraph 1, further comprising a second polynucleotide encoding a reverse transcriptase (RT), or a portion thereof, wherein the encoded RT or portion thereof is capable of synthesizing a DNA copy of at least a portion of the msd locus encoding the msDNA.

Paragraph 3. The engineered nucleic acid construct of paragraph 2,
wherein the second polynucleotide comprises:
III) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide listed in Table A; and/or
IV) encodes a consensus amino acid sequence of Table C, or encodes an amino acid sequence having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an amino acid sequence listed in Table C; and/or
wherein the second polynucleotide encodes:
V) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or
VI) a polypeptide comprising a polypeptide consensus sequence listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an amino acid sequence listed in Table C; and/or wherein the second polynucleotide optionally does not encode an amino acid sequence listed in Table X.

Paragraph 4. An engineered nucleic acid construct comprising:
a) a first polynucleotide encoding a non-coding RNA (ncRNA), said first polynucleotide comprising:
   1) an msr locus encoding the msr RNA portion of a multi-copy single-stranded DNA (msDNA); and
   2) an msd locus encoding the msd RNA portion of the msDNA;
b) one or more heterologous nucleic acids inserted at or within a location selected from: the msd locus, upstream of the msr locus, upstream of the msd locus, and downstream of the msd locus; and
c) a second polynucleotide encoding a reverse transcriptase (RT), or a portion thereof, wherein the encoded RT or portion thereof is capable of synthesizing a DNA copy of at least a portion of the msd locus encoding the msDNA, and,
wherein the non-coding RNA (ncRNA) of the first polynucleotide optionally has a conserved structure of any one of the ncRNA structures of FIGS. 2-27;
wherein the second polynucleotide comprises:
I) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide listed in Table A; and/or
wherein the second polynucleotide encodes:
II) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or
IV) a polypeptide comprising a polypeptide consensus sequence listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table C; and
wherein the second polynucleotide optionally does not encode an amino acid sequence of Table X.

Paragraph 4a. An engineered nucleic acid construct, comprising:
1) an msr locus (that encodes the msr RNA portion of an msDNA);
2) an msd locus encoding the msd RNA portion of the msDNA;
3) a sequence encoding a retron reverse transcriptase (RT), wherein said msd RNA is capable of being reverse transcribed to form the msDNA by the retron reverse transcriptase (RT); and,
4) a heterologous nucleic acid inserted at or within the msd locus, upstream of the msr locus, upstream or downstream of the msd locus;
wherein the engineered nucleic acid construct optionally has (a) a secondary structure of a wild-type ncRNA of any one of FIGS. 2-27 or
b) a variant of a), having:
   i) up to 1, 2, or 3 (e.g., up to 1) nucleotide changes per 10 red lettered-nucleotides;
   ii) up to 4, 5, or 6 (e.g., up to 1 or 2) nucleotide changes per 10 black lettered-nucleotides; and/or
   iii) up to 7, 8, or 9 (e.g., up to 3 or 4) nucleotide changes per 10 grey lettered-nucleotides.

Paragraph 5. The engineered nucleic acid construct of any one of paragraphs 1 to 4a, comprising one or more sequence modifications (e.g., an insertion, deletion, and/or substitution of one or more nucleotide(s)) in the msr locus and/or the msd locus that:
a) modulates (e.g., enhances) reverse transcription, processivity, accuracy/fidelity, and/or production of the msDNA (e.g., in the mammalian cell);
b) modulates (e.g., reduces) immunogenicity of ncRNA encoded by the engineered retron (e.g., the msr locus and/or the msd locus) in a host (e.g., a host comprising the mammalian cell);
c) modulates (e.g., inhibits, either permanently or transiently) a function of the msDNA; and/or
d) modulates (e.g., improves) efficiency of targeted genome editing/engineering.

Paragraph 6. The engineered nucleic acid construct of any one of paragraphs 1 to 4, wherein said engineered nucleic acid construct has a secondary structure of a wild-type retron encoding a wild-type retron ncRNA encompassed by:
a) any one of the structures as depicted in FIGS. 2-27, or
b) a variant of a), having:
   i) up to 1, 2, or 3 (e.g., up to 1) nucleotide changes per 10 red lettered-nucleotides;
   ii) up to 4, 5, or 6 (e.g., up to 1 or 2) nucleotide changes per 10 black lettered-nucleotides; and/or
   iii) up to 7, 8, or 9 (e.g., up to 3 or 4) nucleotide changes per 10 grey lettered-nucleotides; and/or
optionally further comprising:
   i) 7, 8, 9, or 10 (e.g., 9 or 10) nucleotides present per 10 red-circled nucleotides;
   ii) 6, 7, 8, 9, or 10 (e.g., 8, 9 or 10) nucleotides present per 10 black-circled nucleotides;
   iii) 4, 5, 6, 7, 8, 9, or 10 (e.g., 6, 7, 8, 9 or 10) nucleotides present per 10 grey-circled nucleotides; and/or
   iv) 2, 3, 4, 5, 6, 7, 8, 9, or 10 (e.g., 4, 5, 6, 7, 8, 9, or 10) nucleotides present per 10 white-circled nucleotides.

Paragraph 7. The engineered nucleic acid construct of any one of paragraphs 1-6, wherein the nucleic acid construct is engineered by introducing the one or more sequence modifications into a wild-type retron encoding a wild-type ncRNA listed in Table B.

Paragraph 8. The engineered nucleic acid construct of any one of paragraphs 1-7, wherein the one or more sequence modifications in the ncRNA comprises one or more of:
(i) a modified (e.g., mutated, reduced, or eliminated) bulge in a1, a2, or both a1 and a2;
(ii) an extension or shortening of a1, a2, or both a1 and a2;
(iii) an extension or shortening of a spacer sequence between hairpin loops (e.g., S1, S2, S3, and/or S4);
(iv) an additional or modified (e.g., mutated or eliminated) bulge in hairpin loops (e.g., L2 and/or L3 (e.g., by removing unpaired bases in the bulge, or by replacing unpaired bases with an equivalent number of base pairs));
(v) a modified (e.g., extended or shortened) length of hairpin loops (e.g., L1, L2, L3, and/or L4);
(vi) an alternative L1 and/or L2 having complement, reverse, or reverse complement sequences;
(vii) a modified (e.g., increased) number of unpaired bases at the tip of hairpin loops (e.g., L1, L2, L3, and/or L4);
(viii) a modified (e.g., increased or decreased) GC content in hairpin loops (e.g., L1, L2, L3, and/or L4);
(ix) an insertion of the heterologous nucleic acid in spacer sequences between hairpin loops (e.g., S1, S2, S3 and/or S4), or at the tip of hairpin loops (e.g., L1, L2, L3, and/or L4);
(x) a deletion of one or more hairpin loops (e.g., L1, L2, L3 and/or L4);
(xi) an addition of a new loop in a spacer sequence between hairpin loops (e.g., SI, S2, S3, and/or S4);
(xii) circularization of the ncRNA with the 5′ end and the 3′ end of the ncRNA being connected either directly, or via a spacer sequence;
(xiii) a repositioned branching guanosine capable of initiating reverse transcription priming;
(xiv) a staggered end sequence that reduces immunogenicity of the retron ncRNA, created by, e.g., adding or removing the 5′ al nucleotides and/or the 3′ a2 nucleotides; and/or,
(xv) an antisense sequence complementary to a CRISPR/Cas guide RNA (gRNA) sequence encoded by the heterologous nucleic acid, wherein the antisense sequence hybridizes to and inhibits said gRNA in the encoded retron ncRNA, and wherein said antisense sequence is removed upon reverse transcription of the msDNA.

Paragraph 9. The engineered nucleic acid construct of any one of paragraphs 1-8, wherein the one or more heterologous nucleic acid sequences comprise:
a) a heterologous nucleic acid (such as the coding sequence for an RNA aptamer or a ribozyme) inserted into the msr locus or the msd locus (such as in an S region (e.g., S1, S2, S3 and/or S4), or the tip of an L region (e.g., L1, L2, L3 and/or L4), or upstream or downstream of either the msr locus or the msd locus; or
b) a first heterologous nucleic acid inserted into the msd locus, and a second heterologous nucleic acid inserted either upstream of the msr locus or downstream of the msd locus, wherein the second heterologous nucleic acid encodes a guide RNA.

Paragraph 10. The engineered nucleic acid construct of any one of paragraphs 1-9, wherein said heterologous nucleic acid encodes:
(a) a protein or peptide of interest, or wherein said heterologous nucleic acid comprises;
(b) a DNA donor template sequence;
(c) a functional DNA element selected from a promoter, an enhancer, a protein binding sequence, a methylation site, a homology region for assisting gene editing, and the like; or
(d) a coding sequence for a functional RNA element selected from a guide RNA and a ncRNA.

Paragraph 11. The engineered nucleic acid construct of paragraph 10, wherein said protein or peptide of interest comprises a therapeutic protein useful in treating a disease.

Paragraph 12. The engineered nucleic acid construct of paragraph 10, wherein said DNA donor template sequence corrects/repairs/removes a mutation at the target genome site.

Paragraph 13. The engineered nucleic acid construct of any one of paragraphs 1-12, further comprising or encoding a sequence-specific nuclease (such as a CRISPR/Cas effector enzyme, a ZFN, a TALEN, a meganuclease, TnpB, IscB, or a restriction endonuclease (RE)), and/or a DNA-repair modulating biomolecule.

Paragraph 13b. The engineered nucleic acid construct of paragraphs 1-13 wherein the engineered nucleic acid is an all-RNA component system.

Paragraph 13c. The engineered nucleic acid construct of paragraphs 1-13 wherein the engineered nucleic acid is an all-DNA molecule system.

Paragraph 14. The engineered nucleic acid construct of paragraph 13, wherein the sequence-specific nuclease is fused to the RT, optionally via a flexible linker (e.g., a flexible linker comprising Gly and Ser rich sequences such as G4S repeats or GS repeats) or by a generally disordered protein sequence (such as unstructured hydrophilic, biodegradable protein polymer, e.g., an XTEN peptide polymer).

Paragraph 15. The engineered nucleic acid construct of paragraph 13 or 14, wherein the nuclease is a CRISPR/Cas effector enzyme that forms a complex with a guide RNA (gRNA) recognizing a target sequence, wherein the gRNA is linked to the ncRNA and/or the msDNA, either directly or through a linker/spacer polynucleotide.

Paragraph 16. The engineered nucleic acid construct of paragraph 13, wherein the DNA-repair modulating biomolecule is a regulatory protein that modulates (e.g., enhances) HDR, and the regulatory protein is optionally fused to the RT or to the sequence-specific nuclease, optionally via a flexible linker (e.g., the flexible linker comprising Gly and Ser rich sequences such as G4S repeats or GS repeats) or by a generally disordered protein sequence (such as unstructured hydrophilic, biodegradable protein polymer, e.g., an XTEN peptide polymer).

Paragraph 17. A vector system comprising one or more vectors comprising the engineered nucleic acid construct of any one of paragraphs 1-16, wherein the vector system is optionally all-RNA.

Paragraph 18. The vector system of paragraph 17, wherein the msr locus, the msd locus, and the polynucleotide encoding the RT are comprised within the same vector.

Paragraph 19. The vector system of paragraph 17 or 18, wherein the same vector further comprises a promoter operably linked to the msr locus and/or the msd locus.

Paragraph 20. The vector system of paragraph 19, wherein the promoter is further operably linked to the polynucleotide encoding the RT.

Paragraph 21. A vector system comprising one or more vectors, comprising the engineered nucleic acid construct of paragraph 1 or 2, wherein the vector system further comprises a second polynucleotide encoding a reverse transcriptase (RT), or a portion thereof, wherein the encoded RT is capable of synthesizing a DNA copy of at least a portion of the msd locus encoding the msDNA, and wherein the msr locus, the msd locus, and the second polynucleotide encoding the RT are provided by at least two different vectors.

Paragraph 22. The vector system of paragraph 21, wherein:
a) the second polynucleotide comprises:
   i) a polynucleotide listed in Table A, or a polynucleotide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polynucleotide listed in Table A; and/or
b) the second polynucleotide encodes:
   i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or
   ii) a polypeptide listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table C; and
wherein the second polynucleotide optionally does not encode a polypeptide listed in Table X.

Paragraph 23. The vector system of paragraph 21 or 22, wherein the polynucleotide encoding the RT is provided in trans with respect to the msr gene and/or the msd gene.

Paragraph 24. The vector system of any one of paragraphs 17-23, wherein the one or more vectors comprise a viral vector.

Paragraph 25. The vector system of paragraph 24, wherein the viral vector is a retroviral vector, a lentiviral vector, an adenoviral vector, an adeno-associated viral vector, a vaccinia viral vector, a poxviral vector, or a herpes simplex viral vector.

Paragraph 26. The vector system of any one of paragraphs 17-23, wherein the one or more vectors comprise a non-viral vector.

Paragraph 27. The vector system of paragraph 26, wherein the non-viral vector comprises a plasmid.

Paragraph 28. The vector system of paragraph 26, wherein the non-viral vector comprises a liposome, a lipid nanoparticle (LNP), a cationic polymer, a vesicle, or a gold nanoparticle.

Paragraph 29. The vector system of any one of paragraphs 17-28, comprising a vector encoding a sequence-specific nuclease.

Paragraph 30. The vector system of paragraph 29, wherein the sequence-specific nuclease comprises an RNA-guided sequence-specific nuclease (e.g., a CRISPR/Cas effector enzyme, an engineered RNA-guided FokI-nuclease (e.g., dCas-FokI), an RNA-guided DNA endonuclease, TnpB, IscB, or a transposon-associated nuclease), or a non-RNA-guided sequence-specific nuclease (e.g., a meganuclease, a zinc finger nuclease (ZFN), a TALE nuclease (TALEN), or a restriction endonuclease (RE)).

Paragraph 31. The vector system of paragraph 30, wherein the Cas effector enzyme is a Class 1, Type I, II, or III Cas; a Class 2, Type II Cas (e.g., Cas9); or a Class 2, Type V Cas (e.g., Cpf1).

Paragraph 32. The vector system of paragraph 30, wherein: 1) the RNA-guided sequence-specific nuclease comprises the CRISPR/Cas effector enzyme, the engineered RNA-guided FokI-nuclease (e.g., dCas-FokI), the RNA-guided DNA endonuclease, TnpB, IscB, IsrB, or the transposon-associated nuclease; or, 2) non-RNA-guided sequence-specific nuclease comprises the meganuclease, the zinc finger nuclease (ZFN), the TALE nuclease (TALEN), or the restriction endonuclease (RE).

Paragraph 33. The vector system of any one of paragraphs 17-32, further comprising a vector encoding a homologous recombination enhancer protein.

Paragraph 34. An RNA molecule encoded by the engineered nucleic acid construct of any one of paragraphs 1-16.

Paragraph 35. A recombinant retron-based genome editing system comprising:
a) a non-coding RNA (ncRNA) comprising:
   i) an nsr locus encoding the msr RNA portion of a multi-copy single-stranded DNA (msDNA); and
   ii) an msd locus encoding the msd RNA portion of the msDNA;
b) a heterologous nucleic acid inserted at or within a location selected from: the msd locus, upstream of the msr locus, upstream of the msd locus, and downstream of the msd locus; and
c) a sequence encoding a reverse transcriptase (RT), or a domain thereof comprising:
   i) a polypeptide listed in Table A, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table A; and/or
   ii) a polypeptide listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to a polypeptide listed in Table C; and
wherein, the RT optionally does not comprise a polypeptide listed in Table X.

Paragraph 36. A recombinant retron-based genome editing system comprising:
a) a non-coding RNA (ncRNA) comprising:
   i) an msr locus encoding the msr RNA portion of a multi-copy single-stranded DNA (msDNA); and
   ii) an msd locus encoding the msd RNA portion of the msDNA;
wherein the ncRNA comprises:
   i) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B; and/or
   ii) an ncRNA having a conserved structure of any one of the ncRNA structures of FIGS. 2-27; and
wherein the ncRNA optionally excludes any ncRNA associated in nature with any one of the retron reverse transcriptases of Table X;
b) a heterologous nucleic acid inserted at or within a location selected from: the msd locus; upstream of the msr locus; upstream of the msd locus; and downstream of the msd locus; and
c) a reverse transcriptase (RT), or a portion thereof, wherein the RT is capable of synthesizing a DNA copy of at least a portion of the msd locus encoding the msDNA.

Paragraph 37. A recombinant retron-based genome editing system comprising:
a) a non-coding RNA (ncRNA) comprising:
   i) an msr locus encoding the msr RNA portion of a multi-copy single-stranded DNA (msDNA); and
   ii) an msd locus encoding the msd RNA portion of the msDNA;
wherein, the ncRNA comprises:
   i) an ncRNA listed in Table B, or an ncRNA having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an ncRNA listed in Table B; and/or
   ii) an ncRNA having a conserved structure of any one of the ncRNA structures of FIGS. 2-27; and
   wherein the ncRNA optionally excludes any ncRNA associated in nature with any one of the retron reverse transcriptases of Table X;
b) a heterologous nucleic acid inserted at or within a location selected from: the msd locus, upstream of the msr locus, upstream of the msd locus, and downstream of the msd locus; and
c) a reverse transcriptase (RT) or a domain thereof: wherein the RT comprises:
   i) an RT listed in Table A, or an RT having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an RT listed in Table A; and/or
   ii) a consensus sequence listed in Table C, or a polypeptide having at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8% or at least 99.9% sequence identity to an amino acid sequence listed in Table C; and
wherein the RT does not optionally comprise an RT listed in Table X.

Paragraph 38. An isolated host cell comprising the engineered nucleic acid construct of any one of paragraphs 1-16, the vector system of any of paragraphs 17-33, the RNA molecule of paragraph 34, or the recombinant retron-based genome editing system of any one of paragraphs 35-37.

Paragraph 39. The isolated host cell of paragraph 38, wherein the host cell is a prokaryotic, archeon, or eukaryotic host cell.

Paragraph 40. The isolated host cell of paragraph 38, wherein the eukaryotic host cell is a mammalian host cell.

Paragraph 41. The isolated host cell of paragraph 39, wherein the eukaryotic host cell is a non-human host cell.

Paragraph 42. The isolated host cell of paragraph 40, wherein the mammalian host cell is a human host cell.

Paragraph 43. The isolated host cell of any one of paragraphs 38-42, wherein the host cell is an artificial cell or genetically modified cell.

Paragraph 44. A pharmaceutical composition comprising:
a) the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, the recombinant retron-based genome editing system of any one of paragraphs 35-37, and/or the isolated host cell of any one of paragraphs 38-43; and
b) a pharmaceutically acceptable carrier.

Paragraph 45. A pharmaceutical composition comprising:
a) a lipid nanoparticle (LNP); and
b) the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, and/or the recombinant retron-based genome editing system of any one of paragraphs 35-37.

Paragraph 46. The pharmaceutical composition of paragraph 45, wherein the LNP encapsulates the engineered nucleic acid construct, ncRNA, vector system, RNA molecule, and/or engineered nucleic acid-enzyme construct.

Paragraph 47. The pharmaceutical composition of paragraph 45 or 46, wherein the lipid nanoparticle comprises:
a) one or more ionizable lipids;
b) one or more structural lipids;
c) one or more PEGylated lipids; and
d) one or more phospholipids.

Paragraph 48. The pharmaceutical composition of paragraph 47, wherein the one or more ionizable lipids is selected from the group consisting of those disclosed in Table 2.

Paragraph 49. The pharmaceutical composition of paragraph 47 or 48, wherein the one or more structural lipids are selected from the group consisting of cholesterol, fecosterol, beta sitosterol, sitosterol, ergosterol, campesterol, stigmasterol, brassicasterol, tomatidine, tomatine, ursolic acid, alpha-tocopherol, prednisolone, dexamethasone, prednisone, and hydrocortisone.

Paragraph 50. The pharmaceutical composition of any one of paragraphs 47-49, wherein the one or more PEGylated lipids are selected from the group consisting of PEG-c-DOMG, PEG-DMG, PEG-DLPE, PEG-DMPE, PEG-DPPC, and PEG-DSPE.

Paragraph 51. The pharmaceutical composition of any one of paragraphs 47-50, wherein the one or more phospholipids are selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1.2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocho line (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoylsn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin.

Paragraph 52. The pharmaceutical composition of any one of paragraphs 47-51, wherein the lipid nanoparticle comprises about 48.5 mol % ionizable lipid, about 10 mol % phospholipid, about 40 mol % structural lipid, and about 1.5 mol % of PEG lipid.

Paragraph 53. The pharmaceutical composition of any one of paragraphs 47-52, wherein the lipid nanoparticle comprises about 48.5 mol % ionizable lipid, about 10 mol % phospholipid, about 39 mol % structural lipid, and about 2.5 mol % of PEG lipid.

Paragraph 54. The pharmaceutical composition of any one of paragraphs 47-53, wherein the LNP further comprises a targeting moiety operably connected to the LNP.

Paragraph 55. The pharmaceutical composition of any one of paragraphs 47-54, wherein the LNP further comprises one or more additional components selected from the group consisting of DDAB, EPC, 14PA, 18BMP, DODAP, DOTAP, and C12-200.

Paragraph 56. The pharmaceutical composition of paragraph 45, wherein the lipid nanoparticle comprises at least one cationic lipid selected from the group consisting of: a lipid in Table 2, a lipid having a structure of Formula (I), a lipid having a structure of Formula (II), a lipid having a structure of Formula (III), a lipid having a structure of Formula (IV), a lipid having a structure of Formula (V), a lipid having a structure of Formula (VI), and combinations thereof.

Paragraph 57. A kit comprising the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, the recombinant retron-based genome editing system of any one of paragraphs 35-37, the host cell of any one of paragraphs 38-43, or the pharmaceutical composition of any one of paragraphs 44-56, and instructions for genetically modifying a cell with said engineered nucleic acid construct, ncRNA, vector system, host cell, or pharmaceutical composition.

Paragraph 58. A method of modifying a target DNA sequence in a host (e.g., mammalian) cell, the method comprising introducing into the mammalian cell the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, the recombinant retron-based genome editing system of any one of paragraphs 35-37, or the pharmaceutical composition of any one of paragraphs 44-56, to allow production of the msDNA in the host (e.g., mammalian) cell, wherein the heterologous nucleic acid in the msDNA modifies the genome of the host.

Paragraph 59. The method of paragraph 58, wherein the modifying comprises introducing an insertion, deletion and/or substitution into the target DNA sequence.

Paragraph 60. A method of treating a disease or condition in a subject in need thereof, the method comprising administering a therapeutically effective amount of the engineered nucleic acid construct of any one of paragraphs 1-16, ncRNA encoded by the engineered nucleic acid construct of any one of paragraphs 1-16, the vector system of any one of paragraphs 17-33, the RNA molecule of paragraph 34, the recombinant retron-based genome editing system of any one of paragraphs 35-37, the host cell of any one of paragraphs 38-43, or the pharmaceutical composition of any one of paragraphs 44-56 to the subject, thereby treating the disease or condition in the subject.

Paragraph 61. A method of treating a disease or condition in a subject in need thereof, the method comprising administering a therapeutically effective amount of the host cell of any one of paragraphs 38-43 to the subject, thereby treating the disease or condition in the subject.

Paragraph 62. The method of paragraph 61, wherein the host cell is autologous to the subject.

Paragraph 63. The method of paragraph 61, wherein the host cell is allogeneic to the subject.

### Exemplary Embodiment Group C

The following paragraphs also describe exemplary and non-limiting embodiments of the present disclosure.

Paragraph 1. A gene editing system comprising one or more delivery vehicles, wherein:
the delivery vehicle(s) comprise RNA cargo,
said RNA cargo comprises (a) at least one mRNA molecule encoding (i) a nucleic acid programmable nuclease and (ii) a retron reverse transcriptase, (b) an engineered retron ncRNA, and (c) guide RNA for the programmable nuclease,
each delivery vehicle contains (a)(i) and/or (a)(ii) and/or (b) and/or (c),
whereby one delivery vehicle or more than one delivery vehicle delivers (a)(i), (a)(ii), (b), and (c).

Paragraph 2. The gene editing system of paragraph 1, wherein (a)(i) and (a)(ii) comprise a single mRNA molecule encoding the nucleic acid programmable nuclease and the retron reverse transcriptase.

Paragraph 3. The gene editing system of paragraph 2, wherein (a)(i) and (a)(ii) are encoded and expressed as a fusion protein.

Paragraph 4. The gene editing system of paragraph 3, wherein the fusion protein comprises the C-terminal end of the nucleic acid programmable nuclease fused to the N-terminal end of the retron reverse transcriptase (nuclease:RT fusion).

Paragraph 5. The gene editing system of paragraph 3, wherein the fusion protein comprises the N-terminal end of the nucleic acid programmable nuclease fused to the C-terminal end of the retron reverse transcriptase (RT:nuclease fusion).

Paragraph 6. The gene editing system of paragraph 1, wherein (a)(i) and (a)(ii) comprise a first mRNA molecule encoding the nucleic acid programmable nuclease and a second mRNA molecule encoding the retron reverse transcriptase.

Paragraph 7. The gene editing system of paragraph 1, wherein (c) is separate from (a)(i), (a)(ii) and (b) or is provided in trans.

Paragraph 8. The gene editing system of paragraph 1, wherein (b) the engineered retron ncRNA, and (c) the guide RNA are fused or are provided in cis.

Paragraph 9. The gene editing system of paragraph 8, wherein the guide RNA is fused to the 5′ end of the retron ncRNA.

Paragraph 10. The gene editing system of paragraph 8, wherein the guide RNA is fused to the 3′ end of the retron ncRNA.

Paragraph 11. The gene editing system of paragraph 8, wherein the engineered ncRNA comprises a first guide RNA fused to the 5′ end of the retron ncRNA, and a second guide RNA fused to the 3′ end of the retron ncRNA, and the first and second guide RNAs target different sequences.

Paragraph 12. The gene editing system of paragraph 1, wherein the one or more delivery vehicles comprise a liposome or a lipid nanoparticle (LNP).

Paragraph 13. The gene editing system of paragraph 1, wherein (a) the at least one mRNA molecule encoding (i) the nucleic acid programmable nuclease and (ii) the retron reverse transcriptase, and (b) the engineered retron ncRNA are in the same delivery vehicle.

Paragraph 14. The gene editing system of paragraph 1, wherein the (a) the at least one mRNA molecule encoding (i) the nucleic acid programmable nuclease and (ii) the retron reverse transcriptase, and (b) the engineered retron ncRNA are in separate delivery vehicles.

Paragraph 15. The gene editing system of paragraph 1, wherein the nucleic acid programmable nuclease and the retron reverse transcriptase are encoded on separate mRNA molecules and those separate mRNA molecules of (a)(i) and (a)(ii) are contained in the same delivery vehicle.

Paragraph 16. The gene editing system of paragraph 1, wherein the nucleic acid programmable nuclease and the retron reverse transcriptase are encoded on separate mRNA molecules and those separate mRNA molecules of (a)(i) and (a)(ii) are contained in different delivery vehicles.

Paragraph 17. The gene editing system of paragraph 1, wherein the engineered retron ncRNA includes a sequence of interest encoding a donor polynucleotide comprising an intended edit to be integrated at a target sequence in a cell, and wherein the donor polynucleotide is flanked by a 5′ homology arm that hybridizes to a sequence 5′ to the target sequence and a 3′ homology arm that hybridizes to a sequence 3′ to the target sequence.

Paragraph 18. The gene editing system of paragraph 1, wherein the nucleic acid programmable nuclease comprises a Cas9 nuclease, a TnpB nuclease, or a Cas12a nuclease.

Paragraph 19. The gene editing system of paragraph 18, wherein the nucleic acid programmable nuclease comprises a Cas9 nuclease.

Paragraph 20. The gene editing system of paragraph 1, wherein the retron reverse transcriptase has an amino acid sequence of Table A, or an amino acid sequence having at least 90%, 95%, 99%, or 100% sequence identity with any amino acid sequence of Table A.

Paragraph 21. The gene editing system of paragraph 1, wherein the engineered retron ncRNA comprises:
A) a pre-msr sequence having a first complementary region of the retron ncRNA;
B) an msr sequence including an msr stem-loop structure;
C) an msd sequence including an msd stem-loop structure and a sequence of interest, wherein said msd sequence templates a single strand DNA product (RT-DNA) in the presence of the retron reverse transcriptase; and
D) a post-msd sequence having a second complementary region, wherein the first and second complementary regions form an al/a2 duplex region of the retron ncRNA,
   wherein the msr stem-loop structure, the msd stem-loop structure, or the al/a2 duplex comprise a modification which result in increased editing efficiency in the presence of a nucleic acid programmable nuclease that associates with the one or more guide RNAs, and
   wherein optionally one or more of the guide RNAs of (c) are coupled to the pre-msr sequence, the post-msd sequence, or both the pre-msr sequence and the post-msd sequence.

Paragraph 22. The gene editing system of paragraph 21, wherein the sequence of interest encodes a donor polynucleotide comprising an intended edit to be integrated at a target sequence of a cell, wherein the donor polynucleotide is flanked by a 5′ homology arm that hybridizes to a sequence 5′ to the target sequence and a 3′ homology arm that hybridizes to a sequence 3′ to the target sequence.

Paragraph 23. The gene editing system of paragraph 21, wherein the ncRNA has a nucleotide sequence of any sequences of Table B, or a nucleotide sequence having at least 90%, 95%, 99%, or 100% sequence identity with any sequence of Table B.

Paragraph 24. An isolated cell comprising the gene editing system of paragraph 1.

Paragraph 25. The isolated cell of paragraph 24, wherein the isolated cell is a mammalian cell.

Paragraph 26. The isolated cell of paragraph 25, wherein the mammalian cell is a human cell.

Paragraph 27. A composition comprising:
a) the gene editing system of paragraph 1; and
b) a pharmaceutically or veterinarily acceptable carrier.

Paragraph 28. The composition of paragraph 27, wherein the delivery vehicle is a lipid nanoparticle comprising:
a) one or more ionizable lipids;
b) one or more structural lipids;
c) one or more PEGylated lipids; and
d) one or more phospholipids.

Paragraph 29. The composition of paragraph 28, wherein the one or more ionizable lipids comprises an ionizable lipid set forth in Table 2.

Paragraph 30. A method of genetically modifying a cell comprising:
contacting the gene editing system of paragraph 17 with the cell, thereby delivering the RNA cargo to the cell,
   wherein:
      the nucleic acid programmable nuclease forms a complex with the guide RNA, wherein said guide RNA directs the complex to the target sequence,
      the nucleic acid programmable nuclease creates a double-stranded break in in the target sequence,
      the retron reverse transcriptase and engineered retron ncRNA create RT DNA that comprises the donor polynucleotide, and
      the donor polynucleotide becomes integrated at the target sequence.

### Examples

### Example 1: Retron Engineering

This example demonstrates that an engineered (or recombinant) retron as described herein can be engineered based on existing sequence information in a sequence database.

Specifically, retron-like reverse transcriptases (RTs) are first identified from various genomic or metagenomic sequence databases. The identified ncRNA regions of the retrons are then predicted in silico, and/or are determined empirically by, for example, reconstituting the putative retron system in a living cell, to assay for msDNA production.

Once a particular wild-type retron is identified and confirmed, one or more sequence elements of the retron are modified based on the methods described herein, and/or the associated RT is modified or engineered to enhance the overall activity and/or processivity of the retron. For example, the wild-type retron can be engineered by one or more of the following methods: (a) addition of a heterologous nucleic acid sequence of interest (e.g., a nucleotide sequence encoding an HDR donor template) to the various portions and structures of the msd locus; (b) performing any/all of the structural modifications described herein; (c) optionally linking the engineered retron ncRNA to one or more CRISPR gRNA, e.g., a gRNA linked to the 3′ end of a retron ncRNA, or a gRNA linked to the 5′ end of a retron ncRNA, or a pair of gRNAs linked one to the 3′ end and one to the 5′ end of a retron ncRNA.

The engineered retron or its encoded ncRNA are optionally linked to a sequence-specific nuclease such as CRISPR/Cas enzyme and/or gRNA, ZFN, TALEN, TnpBs, or IscBs, and the like

For example, the RT is fused to the CRISPR/Cas enzyme (such as Cas9 or Cpf1) as a N- or C-terminal fusion, optionally further fused to a nuclear localization signal (NLS) at the N- and/or C-terminal of the fusion. The ncRNA or the resulting msDNA after reverse transcription is also linked to a guide RNA (gRNA) at the 5′ or 3′ end, and can be used with the cognate CRISPR/Cas enzyme in the method of the invention.

In another example, the RT is linked to a DNA-repair modulating biomolecule, such as a HDR promoter and/or an NHEJ peptide inhibitor, as described herein above.

### Example 2A: Genome Engineering

This example demonstrates that the engineered retron can be used to introduce a heterologous nucleic acid sequence (e.g., a targeting DNA donor or template) into a host cell genome (e.g., a human cell).

First, a targeting DNA is introduced into the msd portion of the engineered retron, as shown in FIG. 3 as a “marker.” The heterologous nucleic acid sequence is designed such that it is flanked by 10-100 or more base pairs of homologous sequence substantially identical/homologous to the genomic sequence at the target site. Thus, the desired edit on the “marker” is in between the homologous sequence arms, and includes an insertion, a deletion, and/or other mutations.

A sequence-specific nuclease, such as a Cas9 nuclease, forms a complex with a guide RNA that specifically targets a location at or near the desired editing site on the genomic sequence. The nuclease is designed such that it does not cut the target once the edit is properly installed. In this experiment, the Cas9 nuclease is linked to the retron reverse transcriptase (RT) as a fusion protein. The Cas9 gRNA is also linked to the ncRNA or msDNA produced by the engineered retron.

The engineered retron encoding the sequence-specific nuclease (and the fused RT) as well as the ncRNA (and the linked gRNA) are then introduced into a host cell, such as a human cell.

The engineered retron is introduced as part of a plasmid for transfection into the cell, or as part of a viral vector (such as an AAV vector) for infecting the cell.

Alternatively, the transcribed ncRNA (and the linked gRNA) can be formulated in vitro in, for example, the lipid nanoparticle or delivery system of the invention, for direct delivery into the host cell. The sequence-specific nuclease (and the fused RT) can either be separately delivered into the host cell (using transfection of plasmid or infection by AAV, etc.), or delivered with the ncRNA in lipid nanoparticles. For example, the coding sequence (e.g., mRNA) of the Cas9-RT fusion is formulated with the ncRNA in the same lipid nanoparticle, or separately formulated as lipid nanoparticles to be delivered together, either simultaneously or one after another.

Once present inside the cell, the Cas9-RT fusion is translated by the host cell translation machinery, while the ncRNA is transcribed from the engineered retron (if the ncRNA is not directly delivered into the cell). The RT portion of the fusion proceeds to reverse transcribe the ncRNA and convert it to msDNA, which includes the heterologous nucleic acid sequence as the cargo/donor/template. Meanwhile, double-stranded breaks (DSBs) are generated at the target site by the CRISPR/Cas9 nuclease. The cargo/donor/template sequence is then integrated into the host genome at the target site, via host cell DNA repair (e.g., HDR) after the Cas9 nuclease forms the DSB.

The cells of interest are then assayed for correct installation of the edit on the heterologous nucleic acid sequence, either through phenotypic changes that occur due to the edit, or by direct DNA sequencing of the target site, or both.

### Example 2B. Level of Precise Retron Editing in Mammalian Cells is Comparable Between Plasmid-Delivered Retrons Versus RNA-Delivered Retrons

While CRISPR/Cas technology has revolutionized genome editing technology, precise editing of pathologic mutations or insertion of large DNA fragments at specific locations to restore fitness of cells is still at its infancy. Delivery of donor DNA for precise editing is a current bottleneck for therapeutic applications. Current therapeutic ex-vivo and in vivo delivery of donor DNA relies on AAV (adeno-associated virus) transduction. However, AAV manufacturing is complicated and expensive. Additionally, AAV has raised safety concerns in some gene therapy clinical trials. The inventors sought to develop an approach for delivery DNA donors that avoids the drawbacks of AAV-based DNA delivery.

The inventors sought instead to develop an RNA-based approach for delivering DNA donors to cells for carrying out precise editing.

Retrons have been discovered in many bacterial species. These genetic elements are defined by their unique ability to produce an unusual satellite DNA known as msDNA (multicopy single-stranded DNA). The DNA includes a reverse transcriptase (RT)-coding gene (ret) and two contiguous, inverted non-coding sequences (named msr and msd). The ret gene and the non-coding RNA (ncRNA, msr and msd) are transcribed as a single RNA, which is folded into a specific secondary structure. Once translated, the RT binds the RNA template downstream from the msd locus, initiating reverse transcription of the RNA towards its 5′ end, assisted by the 2′OH group present in a conserved branching guanosine residue that acts as a primer. Reverse transcription halts before reaching the msr locus, and the resulting DNA, the msDNA, remains covalently attached to the RNA template via a 2′-5′ phosphodiester bond and base-pairing between the 3′ ends of the msDNA and the RNA template. In this Example, these unique features of retrons were utilized to produce donor DNA for precise genome editing through a validated RNA delivery system and were shown to produce similar levels of precise Cas9-based editing in mammalian cells as compared to plasmid-delivered retrons.

FIGS. 1L and 1M show the results of the first set of experiments. Three different kinds of retrons were evaluated (Eco1-R1, Eco3-R2, Eco5-R3) as to their capability of inserting a 16 base pair insertion at a specific genomic site, the EMX1 gene. mRNA of each reverse transcriptase and the cognate non-coding retron RNA were electroporated into Cas9-expressing HEK293T mammalian cells. The guide RNA of the Cas9 is fused with the non-coding retron RNA to ensure that guide RNA and the in situ generated single strand donor DNA (i.e., the product of reverse transcription of the ncRNA template) are colocalized at the editing target site. As a control, a previously established plasmid system encoding Eco1 reverse transcriptase and its non-coding RNA was lipofected into the same cell line. Edited sequences were PCR amplified from extracted genomic DNA and made into NGS libraries and analyzed by sequencing. The results demonstrate that retron components delivered as RNAs could provide donor DNA template for precise editing as efficiently as those delivered via plasmid transfection.

In the second set of experiments (data not shown), the ratio between the reverse transcriptase mRNA and its non-coding RNA template were optimized. Incremental amounts of non-coding RNA template were combined with a given amount of reverse transcriptase mRNA (tested at two different fixed concentrations of reverse transcriptase mRNA). The results indicate that the ratio between the reverse transcriptase mRNA and its non-coding RNA template can be optimized in order to increase the efficiency of editing. For example, the data show that increasing the non-coding RNA template to a given amount of RT enzyme improved the integration rate of the 16 bp insert.

### Example 3: Computational Discovery of Novel Retrons and Phylogenetic Analysis to Increase Retron Diversity for Genome Editing Applications

Reverse transcriptases (RTs), also called RNA-directed DNA polymerases, are enzymes capable of synthesizing DNA using RNA as a template. Although they are present in the three domains of life and viruses; prokaryotic RTs have been traditionally less explored in comparison with their eukaryotic counterparts. Prokaryotic RTs can be divided into 6 main groups: (1) Group II introns, (2) CRISPR-associated RTs, (3) Diversity Generating Retroelements, (4) Retrons, (5) Abi (Abortive infection) RTs and (6) Unknown Groups of RTs (UG). In the last five years, a burst of research has increased the knowledge about prokaryotic RTs that led to the discovery of novel putative systems with potential antiphage properties, including retrons. In this Example, a systematic search of public databases was performed with the aim of increasing the number and diversity of known retrons for possible use in genome editing applications. As a result, new types of retrons have been identified, and the increase in data has allowed the identification of new associated ncRNAs.

As a first step in this work, a set of known retron RTs was manually curated, trimmed, and aligned to create suitable input, which was then used to train an HMM model for identifying new retron RTs.

Next, this model was applied to existing databases of protein sequences (e.g., nr database from NCBI) to identify potential candidate retron RTs.

As a next step, the identified candidates were then grouped by sequence identity and individual representative sequences from each group were chosen. These RT domains of these representative candidates were then aligned, and an initial phylogenetic tree was built.

Next, using information about other known classes of RTs, the full phylogeny was separated into bonafide retron RT candidates and RTs that could belong to other classes (such as group II introns, DGRs, CRISPR-Cas RTs, etc). A new alignment and phylogenetic tree were then constructed from these verified candidates, as shown in FIG. 28. For all sequences in the new alignment, a matrix of the protein neighborhood was built, indicating which proteins were present in the vicinity of the candidate retron RTs. From there, types and subtypes of retrons were defined based on the RT phylogeny and identity of associated effector proteins. The candidate RT sequences are presented in Table A

To predict ncRNAs proximal to the candidate retron RTs, genomic regions proximal to the candidate retron RTs were extracted, aligned, and analyzed for structural covariance using an iterative convergence model.

ncRNA classification methodology:

To identify what type of ncRNA a sequence may belong to, sequences can be checked against the covariance model of existing ncRNA types. The Infernal suite (http://eddylab.org/infernal) provides tools to do this. Briefly, covariance models can be built from structural alignment of known ncRNAs for each type, then collated into a CM database. From there, new sequences can be searched against the databases to verify whether the sequence fits with any of the represented families.

The resulting retron RT sequences are provided herein at Table A. The resulting and corresponding retron ncRNA sequences are provided herein at Table B.

### Example 4: Design and Demonstration of Retron-Based Editing System in Cells

Current genome editing methods can efficiently disrupt target genes by inserting and deleting genetic information using programmable nucleases. However, gene disruption that does not discriminate mutant from wild type allele impairs not only mutant allele in pathogenesis but also wild type allele in normal physiology of the organism. In Transthyretin (TTR) induced amyloidosis, for example, mutated TTR alleles cause the disease but non-pathogenic wild type allele plays a role in neuro protection and injury response (1). As a single nucleotide exchange is a dominant variant in TTR (2), the vast majority of pathogenic alleles in human differ from their nonpathogenic counterparts by small modifications that require much more precise editing technologies to correct. Homology-directed repair (HDR) stimulated by nuclease-mediated DNA breaks has been widely used to install precise editing. However, HDR relies on the delivery of exogenous donor DNA, that can trigger strong immune response and has been shown inefficient to deliver universally high abundance in recipients (3). Current therapeutical ex-vivo and in vivo delivery of donor DNA relies on AAV (adeno-associated virus) transduction. However, AAV manufacturing is complicated and expensive. Additionally, AAV has raised safety concerns in 30% of 255 complete or ongoing clinical trials surveyed by meta-analysis (4). RNA delivery in contrast, successfully demonstrated its safety, easier manufacturing and efficacy in two SARS-Covid2 vaccines. Based on this amenability of RNA as a cargo, we searched the system, where donor DNA for precise editing could be generated from delivered RNA within cells.

Retrons are defined by their unique ability to produce an unusual satellite DNA known as msDNA (multicopy single-stranded DNA) from RNA inside the cell (5). These bacterial elements are involved in phage defense (6) and consist of a non-coding RNA and a reverse transcriptase (RT) that can reverse transcribe non-coding RNA (ncRNA) into msDNA. Their tightly defined sites of reverse transcription enable to insert donor DNA sequences in non-coding RNA substrate. Moreover, their compact size is amenable to deliver along with programmable nuclease in all RNA format. These features place retron an attractive tool for precise genome editing in therapeutic application.

Independent approach to generate new DNA sequences by reverse transcription in the cell is prime editing and its virus originating RT can achieve up to 44 bp insertion (7). Retron in natural sequence can produce up to 163 nucleotide single strand DNA (8), suggesting that longer insertion in retron mediated editing is feasible. In line with this, newly characterized retrons could insert 100 bp with 6 bp deletion in EMX1 locus of HEK293T cells in S. pyogenes Cas9 nuclease system.

A. Evidence of Retron from Different Clades in Precise Editing

As a first step, retron/retron-like RTs were searched in bacterial genomes. Phylogenetic analysis of the over 7,000 retron/retron-like sequences identified from searches against bacterial genomes was conducted, resulting in the phylogenetic tree of FIG. 28. Non-coding RNA gene was searched in the genomic neighborhood of the retron/retron-like RT sequences for conserved RNA secondary structures resembling characterized msr-msd transcripts: self-hybridizing inverted repeat at the ends, hairpin structures in msr and msd, such as those represented by FIGS. 2-27. Using covariance models and consensus structure detection, ncRNAs were identified with a high degree of confidence in certain RTs. To evaluate gene editing applicability of retron across diverse phylogeny, Eco1, Eco3, Eco5, Aco1, RTX3-2042, 6083 v1 and 6943 were selected for further analysis (FIG. 30). Eco1, Eco3 and Eco5 were previously experimentally validated retrons to show msDNA production (9). Aco1 was recently annotated in the literature but has not been experimentally validated as producing msDNA (10). RTX3-2042, 6083 v1 and 6943 are novel retrons.

A gene editing assay was first performed in plasmid DNA format. Retron elements are assembled in a plasmid, where RT is transcribed under CAG promoter and ncRNA fused at its 3′ end to the 5′ end of a single guide RNA (sgRNA) of Cas9 nuclease under U6 or H1 RNA polymerase III promoter (see FIGS. 31A and 31B). Desired sequence to insert at EMX1 targeting site flanked with homology arm at both sides is inserted within msD stem loop of ncRNA of each retron. As described in FIGS. 32 and 33, the plasmid was transfected to Cas9 expressing human embryonic kidney 293T (HEK293T) cells via lipofection. Three days after transfection, EMX1 target genomic locus was amplified by PCR and sequences were analyzed by next generation sequencing (NGS). The precise edit assayed is characterized as a 10 bp insertion and a 6 bp substitution and its percentage is calculated by CRISPResso 2 analysis among other editing outcomes.

A representative editing outcome is shown in FIG. 35. Eco1 (FIG. 36), Aco1 (FIG. 37), RTX3-2042 (FIG. 38), RTX3-6083 v1 and 6943 (FIG. 39A) showed precise editing efficiency of 0.3%, 0.1%, 0.25%, 0.06% and 0.05% (left graph in FIGS. 36, 37, 38, and 39, respectively). Undesired editing outcome defined as indels that incorporated random nucleotides or deleted sequences near Cas9 cutting site amounted to 50%, 3%, 5%, 2% and 4% (right graph in FIGS. 36, 37, 38, and 39A, respectively). Follow up experiments using the same assay indicated that RTX3_6083v1 and RTX3_6943 generated 3-4 fold more precise edits than Eco1 while indels generated from these two retrons were 2-3 fold lower (FIGS. 39B and 39C). RTX3_2042 showed precise editing at similar frequencies to Eco1 but had more variability than other samples (FIGS. 39B and 39C). These data demonstrated that recently reported (Aco1) and three novel retrons (RTX3-2042, RTX6083 v1, RTX3-6943) enable precise gene editing in human cells, in addition to previously characterized Eco1 (11).

Follow up experiments using the same assay indicated that RTX3-6342S and RTX3-6342L and RTX-1262 generated precise edits 10-20 fold more frequently than Eco1 and approximately 5-10 fold more frequent than RTX3-2042, 6083v1, 6943, and 0637 (see FIG. 39E—left). Furthermore, indels generated using RTX3-6342S, 6342L, 1262, 0637, and 6083v1 were two-fold less frequent compared to Eco1 (FIG. 39E—right). Indels generated using RTX3-2042 were decreased by 80% compared to Eco1 (FIG. 39E—right). Additionally, these retrons all generated precise edits more frequently than another literature validated retron, Eco3, albeit with similar or higher frequencies of indels (FIG. 39E). A small group of retrons were also modified with deletions in the msd stem to assess if precise editing could be improved. We noted that precise editing was increased after shortening the msd stem in six retrons while precise editing was lowered or unchanged in the remaining three retrons (FIG. 57 and FIG. 58).

These data demonstrated that recently reported (Aco1) and six novel retrons (RTX3-0637, RTX3-1262, RTX3-2042, RTX6083 v1, RTX3-6342, RTX3-6943) enable precise gene editing in human cells, in addition to previously characterized Eco1 and Eco3 in gene editing. Furthermore, modifying the msd stem length can alter precise editing but the effects can be retron specific.

B. Evidence of Retron-Mediated Gene Editing in all-RNA System

To establish RNA-based editing system, gene editing assay with two RNA components (RT mRNA and ncRNA fused to sgRNA of Cas9 nuclease) was performed first, in HEK293T cells constitutively expressing Cas9 nuclease. With RT and ncRNA being separated in RNA format, the condition for higher precise editing and lower indels could be easier identified than when they are together in the same plasmid by optimizing the ratio between RT enzyme and its substrate. EcoI, Eco3, and Eco5, three previously known retrons, were chosen for this first set of experiments with an all-RNA format.

RT mRNA of each retron and their cognate ncRNAs fused to sgRNA of Cas9 nuclease were produced by in vitro transcription. Following experimental scheme in FIG. 40. Eco1, Eco3 and Eco5 mediated gene editing activity was compared at two different ratios of RT mRNA and ncRNA-sgRNA fusion in Cas9 expressing HEK293T cells. More amount of ncRNA than RT mRNA was transfected to maximize msDNA production for precise gene editing. Results showed precise edits up to 0.4% for Eco3 and as low as 10% indels for Eco3 (FIG. 41). Precise editing frequency in RNA based assay is comparable to that in plasmid-based assay.

Given that Eco3 produced highest editing, RNA load and RT mRNA: ncRNA-sgRNA fusion ratio was further optimized in Eco3 retron system. To 0.2 or 0.5 μg of RT mRNA, ncRNA-sgRNA fusion was added at ratios of RT mRNA to ncRNA, 1:2, 1:3, 1:4, 1:5, 1:8, 1:10, respectively. 0.5 g of RT mRNA gave rise to more precise editing than 0.2 ug at any ratio and increasing ncRNA correlated with higher precise editing (FIG. 42, left). Indels was as low as 6% (FIG. 42, right). With two RNA components, precise editing was achieved up to 1% of cell population.

Next, Cas9 mRNA was added to RT mRNA and ncRNA-sgRNA fusion, making all RNA editing system in HEK293T cells (FIG. 43). At the optimized RNA load and ratio of RT mRNA and ncRNA-sgRNA fusion, the amount of Cas9 mRNA was titrated with Eco3 retron (FIG. 44). Precise editing was observed up to 0.1% of cell population with 0.2 μg of Cas9 mRNA and adding more it did not increase editing efficiency. While the precise editing frequency is an order of magnitude lower than two RNA system, the editing occurred by specific action of Cas9 nuclease and retron, since the absence of either abrogated the precise editing.

The delivery of all RNA system to cells was also made via lipofection using MessengerMAX reagent (FIG. 45). This approach is more resembling in vivo delivery of therapeutic RNAs loaded in lipid nanoparticle (LNP), in terms of formation of RNA/lipid complexes and cell uptake mechanism. When applied to Aco1 retron system that consists of Aco1 RT mRNA, Aco1 ncRNA-sgRNA fusion, and Cas9 mRNA, precise editing of 56 bp insertion with 6 bp deletion was observed in 0.1% of cell population and the frequency of indels was about 1.5% (FIG. 46). Aco1 retron was recently annotated but has not been experimentally validated yet (10). The precise gene editing mediated by Aco1 strongly suggests that Aco1 retron could produce msDNA inside cells. Moreover, the length of insertion in precise editing exceeds those made by other reverse transcriptase mediated editing (7).

C. Enhanced Retron-Mediated Gene Editing in all-RNA System with sgRNA Spike-In

In all experiments above, retron ncRNA fused with sgRNA of Cas9 nuclease was used. This fusion RNA serves as a template of RT and at same time sgRNA portion could complex with Cas9 nuclease. Two enzymes acting on a single RNA piece could create steric effects that affect the activity of either enzyme. In line with this hypothesis, frequency of indels that tells overall Cas9 nuclease activity is only about 1.5% with 400 ng of ncRNA-sgRNA fusion (FIG. 46, right).

To test systematically the hypothesis, Cas9 nuclease activity by frequency of indels was compared between equimolar amounts of ncRNA-sgRNA fusion and separated sgRNA. When electroporated with 200 ng of Cas9 mRNA, 1 μg (7.5 μmol) of ncRNA-sgRNA fusion shows 20-fold lower activity than 0.266 μg (7.5 μmol) of separated sgRNA alone (FIG. 47) The result suggests that ncRNA-sgRNA fusion might inhibit either formation of complex with Cas9 protein or the activity of Cas9-sgRNA complex or both. In addition, chemically modified sgRNA shows 6-fold higher activity than unmodified sgRNA. These results indicate that affected Cas9 cleavage activity by ncRNA-sgRNA fusion could limit precise editing and addition of separated sgRNA could compensate for it. Modification of separated sgRNA is expected to further enhance precise editing.

FIG. 48 summarizes the assay of sgRNA spike-in in all RNA system and FIG. R. shows the results using Eco3 retron. At 0.2 μg of Cas9 mRNA and 0.5 μg of RT mRNA, the amount of sgRNA spike-in is titrated at 50, 100, and 200 ng (FIG. 41, left). The titration was performed at two different ratios of RT mRNA: ncRNA-sgRNA fusion=1:6 or 1:8. With addition of 50 ng of sgRNA spike-in to 1:6 ratio, the precise editing was achieved 40-fold and gradually increased with more sgRNA, reaching up to 50-fold increase at 200 ng. 1:8 ratio of RT mRNA:ncRNA-sgRNA fusion responded similarly to sgRNA spike-in and obtained slightly higher activity than 1:6, up to 13% of precise editing. Higher precise editing with sgRNA spike-in came along with higher indels on the right graph.

The effect of sgRNA spike-in in precise editing was confirmed using orthogonal delivery method, lipofection (FIG. 50). Although overall amount of RNA to transfect was five times lower than in electroporation, 3.4% of precise editing was obtained without sgRNA spike-in, which is 7.5-fold higher than that achieved by electroporation (FIG. 49 and FIG. 51, left graph). With addition of sgRNA as low as 2 ng, precise editing further increased up to 3.5-fold, reaching 12% efficiency (FIG. 51, left). Increasing the amount of sgRNA up to 10 ng did not further change the efficiency. The precise editing observed is dependent on retron since the absence of retron components (RT mRNA and ncRNA-sgRNA fusion) completely abrogated the editing.

These data support that physical fusion of ncRNA and sgRNA of Cas9 nuclease could be a limiting factor for Cas9 activity and consequently precise editing and spike-in of sgRNA significantly enhances editing by complementing it.

D. Separation of ncRNA and sgRNA

The impact of separating sgRNA from ncRNA on editing was tested in Eco3 retron system comparing the editing efficiency between ncRNA-sgRNA fusion and separated ncRNA and sgRNA side by side (FIG. 52). Increasing amount of sgRNA was added to 300 ng of ncRNA that is equimolar amount of 400 ng of ncRNA-sgRNA fusion. With no sgRNA added, no precise editing was observed as expected. With 10 ng of sgRNA, precise editing peaked at 2.23% compared to 1.78% obtained with ncRNA-sgRNA fusion (FIG. 52, left). The increasing amount of sgRNA beyond 10 ng did not further improve precise editing. Frequency of indels (FIG. 52, right) showed a similar trend as precise editing.

These data demonstrates that separation of ncRNA and sgRNA could achieve comparable or even higher precise editing than ncRNA-sgRNA fusion.

E. Lead Retrons from Different Clades Show Precise Editing Activity in an Alternative Cell Type

These examples have shown robust precise editing activity in 293T cells. To confirm that these results can be extended to other cell types, a plasmid encoding lead retron reverse transcriptases and ncRNA/EMX1 sgRNA fusions were co-electroporated, along with plasmid encoding Cas9-mCherry, into K562 cells, an erythroblast leukemia cell line (FIG. 87). FIG. 88 (left) shows that RTX3_2042, 6083v1, 6943, 1262, 6342L, and 6342S can generate precise editing frequencies of 1.3%, 1.2%, 1%, 2.9%, 5%, and 6.7% respectively. The precise editing frequency of RTX3_6342S could be boosted to 10.5% by including an EMX1 gRNA expression cassette on the Cas9-mCherry plasmid. This system also generated robust indel frequencies in all samples. These frequencies varied between retrons and did not correlate with precise editing frequencies (FIG. 88, right). The lack of correlation between indels and precise edits could be explained by steric effects in the ncRNA/sgRNA fusion that influence interactions between Cas9 and gRNA or reverse transcriptase and ncRNA.

The precise editing efficiencies using our novel retrons were also compared to several experimentally validated retrons (10) in this system using the Cas9-mCherry EMX1 gRNA plasmid. Retrons Eco1, Eco3, Aco1, Saul, Sen1, RTX3_2781, RTX3_1262, RTX3_6342L, and RTX3_6342S were tested and observed having precise editing efficiencies of 0.1%, 0.8%, 14%, 3.4%, 0.1%, 14.2%, 19%, and 19.5%, respectively (FIGS. 89 and 90, left). Indels also varied between samples and did not correlate with precise editing levels, which could be explained by the same steric effects mentioned earlier (FIGS. 89 and 90, right). These data indicate that RTX3_1262, 6342L, and 63425 generate precise edits as or more frequently than previously documented retrons.

### F. Lead Retrons can Generate Precise Edits Robustly Using an RNA-Guided Nickase

Previous experiments have shown that retrons can install precise edits using double-strand breaks (DSBs) as recombination initiating events. However, using DSBs to initiate repair can lead to indel formation at off-target sites and chromosomal rearrangements, an undesirable effect for therapeutic development (12). To avoid indels, a ssDNA nick can be used to initiate recombination rather than DSBs (13). To determine if the Cas9 D10A nickase could initiate retron-mediated precise editing, a Cas9 D10A-mCherry EMX1 gRNA plasmid was co-electroporated with a plasmid encoding a retron RT and ncRNA/EMX1 sgRNA fusion into K562 cells. Significant editing was not observed when using retrons Eco1, Eco3, RTX3_6083v1, or RTX3_6943 (FIG. 91, left). However, RTX3_2042, RTX3_1262, RTX3_6342L, and RTX3_6342S saw precise editing frequencies of ˜0.2%, ˜0.6%, ˜0.2%, and ˜0.4% respectively (FIG. 91, left). As expected, precise editing frequencies initiated by a nick were >ten-fold lower than those initiated using a DSB and indel frequencies were much lower compared to the frequencies seen using the Cas9 nuclease (FIG. 91, right). These data suggest that RNA-guided nickases can install edits encoded by novel retrons using a plasmid-based system in vitro.

G. Retron-Mediated Gene Editing is Compatible with LbCas12a and TnpB Nucleases

The examples have used Cas9 nuclease or nickase to perform retron-mediated gene editing. However, alternative nucleases, such as, but not limited to, LbCas12a (14) and TnpB (15) could be used in a retron-based gene editing system in place of a Cas9 nuclease or nickase. To determine if Cas12a or TnpB could induce retron-mediated gene editing, a plasmid encoding LbCas12a, TnpB, or Cas9 was co-electroporated and their corresponding EMX1 gRNAs and a plasmid encoding retron RTX3_6083v1 reverse transcriptase and ncRNA template for each nuclease into K562 cells. Using this approach, it could be noted that LbCas12a and RTX3_6083v1 could mediate precise editing of ˜0.6% (FIG. 92, left) with about 30% less indels than the Cas9 comparator (FIG. 92, right). However, precise editing frequencies generated by TnpB and RTX3_6083v1 were ˜0.04% with one guide and undetectable with another (FIG. 93, left). This is likely due to limited cutting activity with gRNA #2 as no indels were detected above background. Despite similar indel frequencies seen using Cas12a (FIG. 93, right) with sgRNA #1 precise editing frequencies were substantially lower than those seen with Cas12a (FIG. 92, left). The discrepancy in precise editing frequencies could be due to differences in DSB formation, DSB resolution, or protein expression of the nucleases. Additionally, it was noted that precise editing frequencies using a separated Cas9 gRNA and retron ncRNA led to precise editing efficiencies similar to those seen when ncRNA/sgRNA fusions are co-electroporated with a plasmid encoding Cas9 and an EMX1 gRNA expression cassette in prior experiments (FIG. 91, left). In all, retron-mediated gene editing can be achieved with Cas12a-like and TnpB-like nucleases and fusion of gRNA to the retron ncRNA is not necessary to facilitate precise editing using a plasmid based retron gene editor.

H. Retron-Mediated Gene Editing can be Performed with Truncated RT Proteins

Retrons contain partner domains that are potentially critical for physiological phage defense functions but unnecessary for reverse transcriptase activity (6). RTX3_6342 contains an N-terminal region consisting of several alpha helices that are not predicted to align to the reverse transcriptase domain (FIG. 94) (16). To determine if this region was necessary for precise editing, N-terminal truncations of different lengths were created and precise editing activity was assessed by NGS. It is noted that deletions of residues 1-49 AA, 1-99 AA, and 1-144 AA did not compromise the precise editing activity of RTX3_6342 (FIG. 95). As expected, indels were unaffected (FIG. 95). These data suggest that the non-RT domains in 6342S are not necessary for reverse transcription.

### I. Retron-Mediated Installation of Exon-Sized 200-400 Base Pair Inserts

To determine if retron-mediated gene editing could install edits larger than 10 bp, edits of 10, 25, 205, 305, and 405 bp at the EMX1 locus were attempted. Precise editing efficiencies of ˜35%, ˜40%, ˜30%, ˜35%, and ˜40% were observed for 10 bp, 25 bp, 205 bp, 305 bp, and 405 bp, respectively, using RTX3_6342 (FIG. 96, left). RTX3_1262 was also tested in a similar manner and observed as having precise editing efficiencies of ˜22%, ˜35%, ˜28%, ˜2% and ˜10% for edits of 10 bp, 25 bp, 205 bp, 305 bp, and 405 bp, respectively. It is noted that indel frequencies were similar across samples and therefore nuclease activity doesn't limit the installation of these edits (FIG. 96, right). Together these data indicate that inserts >205 bp can be efficiently installed using retron-mediated editing, which exceeds the sizes installed by prior reverse transcriptase-based gene-editors (7). Furthermore, RTX3_6342S tends to install longer edits at more robust frequencies than RTX3_1262.

Reverse transcriptases (RTs) tend to be observed as error-prone compared to replicative DNA polymerases due to lack of a proofreading domain and structural differences in the enzyme active site (17). To determine if longer edits were installed accurately using RTX3_6342S reverse transcriptase, the prior data was re-analyzed using CRISPResso2 parameters that quantified substitutions in edited alleles. It is noted that the percentage of precise edits declined as the inserts became longer when substitutions were quantified using CRISPResso2 (FIG. 97, left, compare circles to triangles). This trend was not observed when quantifying indels (FIG. 97, right, compare circles to triangles) suggesting that reverse transcriptase fidelity is responsible for the mutations seen in precise edits installed by RTX3_6342S. This suggests that further optimization of reverse transcriptase fidelity can be performed to accurately install large edits.

### J. Rational Mutagenesis of Retron RTs to Improve Processivity and Fidelity

The biochemical properties of many RTs, such as processivity and fidelity, are extensively characterized in the literature (17). Furthermore, some RTs have been extensively engineered for high processivity and fidelity for use in applications such as RNA-Sequencing library preparation and prime editing (17) (18). However, Retron RT fidelity and processivity have not been characterized, nor has anyone attempted to improve these traits. The palm, fingers, and thumb domains inside most DNA polymerases interact with the template molecule and direct incoming nucleotides to the catalytic core for efficient DNA synthesis (19). Here select residues in these domains were mutated based on structural comparison of an Alphafold predicted structure of RTX3_6342S with crystal structures of MMLV or Marathon RT and Eco1 (Table J1, below).

TABLE J1
Mutation of select residues in RTX3_6342S.

| Region | MMLV | Eco1 | 6342 | MMLV | Eco1 | 6342 |
|---|---|---|---|---|---|---|
| Thumb | 301 | 279 | 465 | ARG | GLU | ASN |
| Thumb | 302 | 280 | 466 | GLU | HIS | GLU |
| Thumb | 304 | 282 | 468 | LEU | ARG | SER |
| Thumb | 306 | 284 | 470 | THR | TRP | MET |
| Thumb | 308 | 286 | 472 | GLY | SER | SER |
| Thumb | 309 | 287 | 473 | PHE | PHE | TRP |
| Thumb | 311 | 289 | 475 | ARG | LEU | LYS |
| Thumb | 312 | 290 | 476 | LEU | SER | SER |
| Thumb | 313 | 291 | 477 | TRP | VAL | VAL |

These residues were prioritized based on their proximity to the RNA/DNA template present in MMLV/Eco1 structures and prior engineered MMLV or Marathon variants referenced in the literature (17) (20).

As proof of concept, 32 RT variants were engineered across nine residues in the RTX3_6342 RT thumb domain. In particular, the following 32 RT variants were constructed based on RTX3_6342S:
(1) E466K, E466N, E466Q, E466R;
(2) K475N, K475Q, K475R;
(3) M470K, M470N, M470Q, M470R;
(4) N465K, N465Q, N465R;
(5) S468K, S468N, S468Q, S468R;
(6) S472G, S472P;
(7) S476K, S476N, S476Q, S476R;
(8) V477F, V477I, V477L, V477W; and
(9) W473F, W473K, W473R, W473Y.

These constructs were then co-electroporated into K562 cells along with a plasmid encoding Cas9, an EMX1 sgRNA, and the RTX3_6342S ncRNA with a 10 bp or 305 bp insert. After 72 hrs, the cells were harvested for amplicon sequencing analysis and editing quantified using CRISPResso. Most rationally designed mutations modestly decreased the ratio of precise edits to indels (PEIR) using a 10 bp or 305 bp insert (FIG. 98, top). However, it is noted that N465K increased the PEIR while installing a 305 bp insertion, suggesting that the mutation increased the processivity or total ssDNA production of the RTX_6342 reverse transcriptase (FIG. 98, bottom). It was observed that literature mutations improving MMLV activity (T306K and W313F) decreased the PEIR for both 10 bp and 305 bp inserts using orthologous mutants of RTX3_6342 (M470K, and V477F) both as single and combo mutants (FIG. 98 and FIG. 99). It is also noted that RTX3_6342 V265P, an ortholog of MMLV L139P, decreased the PEIR of a 305 bp insert, had no effect on a 205 bp insert, and increased the ratio using a 405 bp insert (FIG. 99). These results could be due to differing sequence characteristics or secondary structures present in each individual insert. A second set of mutations was also screened based on prior engineered marathon RT variants or favorable Rosetta scores after modeling the 6342 RT. It was observed that the mutations E238R, E479K, and K255P all resulted in increases in PEIR over WT 6342 RT (FIG. 100). An additional N-terminal truncation was constructed that preserves an α-helix that potentially makes contact with the retron RNA (FIG. 101). It is noted that the precise edits:indel ratios while installing longer edits were increased using this partial truncation, rather than eliminating the N-terminal domain entirely (FIG. 102).

### K. Protein Fusions to Improve Retron RT Processivity

DNA polymerase processivity has not only been optimized through mutagenesis of the polymerase itself but domain fusions are also known to improve processivity (19). The small DNA binding proteins Sso7d and Sac7d have been fused to MMLV RT to improve processivity during PCR reactions nearly eight-fold on both DNA and RNA templates. Furthermore, attempts were made to improve the editing efficiencies of prime editors by fusing these proteins to the MMLV RT but no significant improvement in editing efficiency was observed (18). However, the edits installed in these experiments were small <90 bp and native MMLV has a processivity of 69±14 bp/binding event suggesting that much longer edits would need to be installed to observe an improvement (18) (19). This specification teaches and demonstrates that edits as long as 400 bp can be installed using retron-mediated genome-editing. To determine if fusing Sso7d or Sac7d could improve retron-mediated genome editing efficiencies, two variants each of Sac7d or Sso7d were fused to retrons RTs from RTX3_1262 and RTX2_6342, at the N or C-terminus. The installation of a 405 bp insertion into the EMX1 locus was then evaluated. It was observed that fusing these domains to the N-terminus of either RT tended to be more robust than C-terminal fusions. Furthermore, these N-terminal fusions led to an approximately ten-fold increase in the installation frequencies of a 405 bp insert with RTX3_1262 and approximately a two-fold increase using RTX3_1262 (FIG. 103). It is thought that this difference in magnitude could be due to differences in the baseline processivity or ssDNA generation of RTX3_6342 (˜4%) vs RTX3_1262 (˜0.4%) and the effect of these fusions on RTs like RTX3_6342 might be more pronounced with longer inserts.

L. Identifying Alternative Retrons from the RTX3_6342 Family

In addition to engineering retrons, screening additional retrons in the same family as certain high performing retrons (e.g., RTX3_6342, 6943, 6083, and 1262) could identify novel retrons with more ideal characteristics for gene-editing. An additional 99 unique retrons were chosen from an initial dataset with at least 70% similarity to RTX3_6342, 6943, 6083, or 1262 RT or their corresponding ncRNAs. These retrons were cloned into plasmid vectors expressing the RT from the CAG promoter and the ncRNA as a ncRNA-gRNA fusion designed to insert 305 bp into the hEMX1 locus. This plasmid was co-electroporated into K562 cells with a second plasmid containing Cas9 and the same hEMX1 sgRNA to boost editing efficiencies. After 72 hrs the genomic DNA was isolated and the hEMX1 locus amplified for sequencing. It was observed that all retrons in this dataset capable of precise editing frequencies >1% were similar to the lead retron RTX3_6342, indicating this family is particularly enriched for RTs compatible with retron-mediated gene-editing (FIG. 104). However, none of these RTs showed precise editing frequencies on par with RTX3_6342.

### Example 5: Improved ncRNAs Generated by Transcription from Template Linearized Plasmid Having Blunt Ends Show Higher Precise Editing Efficiency

Previously made in vitro transcription experiments to produce ncRNA used a double-stranded DNA template containing a 3′ overhang (on same strand as T7 promoter sequence). A new template with a blunt end was designed and tested. See FIG. 52. As shown in FIG. 53, four RNAs (cas9 mRNA, Eco3 RT mRNA, gRNA targeting EMX1 locus and ncRNA) were transfected into 293T cells using MessengerMAX lipofection reagent. Cells were harvested after 3 days, genomic DNA isolated, the EMX1 locus amplified, the Illumina libraries produced and sequenced on NextSeq. The percent of precise edits improves more than 5-fold for ncRNA produced using blunt end template as compared to the precise editing by ncRNA produced using overhang template.

### Example 6: Improved ncRNAs Due to Improved In Vitro Template Optimization and ncRNA End Protection with MS2 Hairpin

Additional elements can be added to the ncRNA by encoding them on the plasmid template. FIG. 53 shows a modified Eco3 ncRNA that comprises an MS2 stem loop hairpin structure added to its 3′ end. As shown in FIG. 54, the precise editing of ncRNA comprising the 3′ MS2 structure resulted in nearly 15-fold increase in precise editing compared to an ncRNA generated from a overhang template, and nearly 3-fold increase in precise editing compared to an ncRNA generated from a blunt end template.

FIG. 53 schematic describes the modification made to improve RNA quality. First approach was about how to linearize plasmid DNA template for in vitro transcription (FIG. 53, A). Two identical plasmids encoding Eco3 ncRNA were made except different restriction enzyme sites at 3′ of ncRNA: One generates 3′ overhang and the other generates blunt end. Templates with 3′ overhangs can cause the RNA polymerase to extend on the opposite strand, resulting in longer transcripts containing the sequence of the complementary strand (13). Second consideration was to add a MS2 hairpin at 3′ of ncRNA (FIG. 53, B). Retron ncRNA contains mutually reverse complementary sequences at 5′ and 3 ends that forms a stem loop structure. However, these sequences could also mediate intermolecular interactions, creating higher-order structures. The addition of MS2 hairpin at one end could mitigate such intermolecular interactions. MS2 sequence is a bacteriophage-derived RNA aptamer that does not interact with endogenous proteins in mammalian cells.

Testing these new RNAs in gene editing assay is shown in FIG. 54. Four components of all RNA system (RT mRNA, Cas9 mRNA, ncRNA, sgRNA) were delivered to HEK293T cells by Lipofectamine MessengerMAX. All RNA was transfected at a fixed amount as shown on the graphs. Using RNA with the template containing a 3′ overhang produced 1.35% precise edits. Using the template containing a blunt end increased precise editing to 5.94%. Adding an MS2 stem loop to the 3′ end of the ncRNA further increased precise editing to 12.39%. On the right graph, frequency of indels is shown for respective conditions. Altogether, precise editing increased nine (9) fold by optimizing in vitro transcription template and 3′ end protection by MS2 hairpin.

### Example 7: Modified ncRNAs with Caps and/or Tails

ncRNA performance can be modified by adding a cap structure to the 5′ end and/or adding a poly(A) tail at the 3′ end, as depicted in FIG. 55. As shown in FIG. 56, using ncRNA with either or both protection by cap and tail lowered indels as compared uncapped/untailed ncRNAs. In this experiment, a 4 component all-RNA system (RT mRNA+Cas9 mRNA+ncRNA-sgRNA+sgRNA) was delivered to HEK293T cells by Lipofectamine MessengerMAX. All RNA was transfected at a fixed amount RT mRNA 100 ng, ncRNA-sgRNA 400 ng, Cas9 mRNA 100 ng, and sgRNA 5 ng. ncRNA-gRNA fusion was either capped (+cap −tail) or poly-A tailed (−cap +tail) or both capped and poly-A tailed (+cap +tail). Using RNA without end protection (−cap −tail) produced ˜4.5% precise edits and the editing was dependent on retron since the absence of RT abrogated precise editing. Using RNA with either or both protection by cap and tail produced lower precise editing (left graph) but lowered indels (right graph) than without cap and tail.

### Example 8: Precise Editing with Cas9 Nickase

Wild Type (WT) Cas9 nuclease generates double strand break (DSB) that can potentially increase genomic instability by generating larger deletion or chromosome translocation. Moreover, DSB induces p53-mediated DNA damage response pathway that often compromises the physiology of edited cells. Single point mutations in either of Cas9 nuclease domains can generate nickases that cut only one strand of targeting site. D10A variant cuts target strand using intact HNH nuclease domain and H840A variant cuts non-target strand using intact RuvC domain. Nicks can initiate homology-directed repair (HDR) and cause less mutagenic end joining than DSBs. These features draw a considerable interest in using nickases in retron-mediated precise editing.

FIG. 73 shows the results testing Cas9 nickases activity in 4 component all-RNA system (RT mRNA+Cas9 mRNA+ncRNA+sgRNA) delivered to HEK293T cells by Lipofectamine MessengerMAX. D10A and H840A nickases were used in parallel with WT Cas9 and they were tested either with separated ncRNA or with ncRNA-sgRNA fusion. All RNA was transfected at a fixed amount RT mRNA 100 ng, ncRNA/ncRNA-sgRNA 400 ng, Cas9 variant mRNA 100 ng, and sgRNA 5 ng. Precise editing by Cas9 WT (Wild Type) was ˜9% when either ncRNA or ncRNA-sgRNA was used with additional sgRNA. With Cas9 H480A mutant that nicks non-target strand, activity was barely seen above background in any condition. With Cas9 D10A mutant that nicks target strand, precise editing was observed ˜0.1% frequency in both separated and fused ncRNA and sgRNA format. Indels are shown at the right graph at respective conditions of left graph. As expected, indels are extremely low for either nickases with or without additional sgRNA and only WT Cas9 generated significant indels.

FIG. 74 shows negative controls that lack one of components (Cas9 variant or ncRNA/ncRNA-sgRNA or RT) in all RNA system. They were tested with or without additional sgRNA. All controls in top graph do not show precise editing activity beyond background level, supporting that precise editing achieved by Cas9 WT or Cas9 nickase is dependent on each component of all RNA system.

### Example 9: Dual sgRNAs Achieve Higher Precise Editing than Single sgRNA

It was reasoned that use of multiple sgRNAs at the proximity of targeting site could generate more free DNA ends upon cleavage and recruit more repair machinery, that would provide higher chance of precise editing. Two pairs of sgRNA were found to test this hypothesis, sgRNA1 at immediately left side of insertion in combination with either sgRNA2 or sgRNA3 at the right side of insertion on EMX1 gene (FIG. 68 schematic at right). Results of testing dual sgRNAs vs single sgRNA was shown in FIG. 37. RTX2042 retron in all RNA system was delivered to HEK293T cells by lipofection. At given amount of RT mRNA, Cas9 mRNA and ncRNA-sgRNA, additional sgRNA was added. The same ncRNA-sgRNA with template encoded a 25 bp insertion on the Cas9 sgRNA's non-targeting strand was used for all conditions. With one additional sgRNA (#1), precise editing was 0.27%, which increased up to 5-fold to either 1.33% (#2) or 1.13% (#3) in the presence of a second sgRNA. On the bottom graph, frequency of indels is shown for respective conditions.

These data demonstrates that dual sgRNA at the vicinity of targeting site could enhance the precise editing mediated by retron.

### Example 10. Editing with Larger Insert Size

Most of experiments so far tested 10 bp insertion at 3′ of EMX1 gene by retron-mediated precise editing. The feasibility of longer insertion was examined by designing new ncRNAs with gradually increased insert length: 25, 50, 75 and 100 bp for Eco3, Aco1, RTX-2042, and RTX-6943 retrons. Sequences to insert are randomly chosen that they do not share any homology with human genome and GC content is 50% for all inserts to generate no bias. At given amount of RT mRNA (100 ng) and Cas9 mRNA (100 ng), 400 ng of ncRNA-sgRNA fusion with different template lengths ranging from 10 to 100 bp were added and delivered to HEK 293T cells via lipofection. Overall, the efficiency is comparable cross the size of insert for all retrons. For 100 bp insertions, precise editing was achieved at 1.04% for Eco3, 1.52% for Aco1, 1.37% for RTX2042 and 0.05% frequency for RTX6943 retron (FIG. 59A). On the bottom row of graphs, frequency of indels for respective conditions shows a very similar trend to precise editing (FIG. 59B).

When sgRNA spike-in was added to above conditions (FIGS. 60A and 60B), the overall activity increased and activity was not compromised with longer inserts. For 100 bp insertions, precise editing increased 1.8˜8 fold from without sgRNA spike-in, 1.82% for Eco3, 4.50% for Aco1, 4.40% for RTX-2042 and 0.4% for RTX-6943. At the bottom panel, frequency of indels also increased for respective conditions.

In combination with Cas9 nuclease, retron can insert 100 bp precisely at target site of the genome up to 4.5% efficiency.

### Example 11. Inserting a Whole Gene by Retron-Mediated Precise Editing

Correcting large mutation hot spot or insert a moderate size of gene precisely into the genome broadens the applicability of Retron-mediated gene editing. To interrogate this, a GFP gene with its own promoter and poly-A signal (1267 bp, FIG. 61A) was integrated in ncRNA of retron and gene editing assay was performed with three retrons, Eco3, Aco1 and RTX6943. As a readout of insertion, 5′ and 3′ junctions between EMX1 host and insert sequences were amplified by qPCR. ΔCt (Ct of without RT sample−Ct of with RT sample) value was used for relative quantification of integration. The bigger value of ΔCt indicates the higher frequency of insert in the sample. With all three retrons, amplicons of both 5′ and 3′ junctions are detectable significantly above background level (FIG. 61B). Amplicon of 3′ junction is more abundant than that of 5′ junction probably because insertion at 3′ is facilitated by enriched 5′ end of msDNA from reverse transcription. FIG. 61D shows PCR products from Aco1 retron analyzed by Agilent Tapestation High Sensitivity D1000 screentape. In the presence of RT (+RT sample, left on the gel), the expected size of amplicons were detected at both 5′ and 3′ junctions of edited alleles as indicated by arrows. Edited alleles were not detected in the absence of RT (−RT sample, right on the gel). Triangle indicates the expected amplicon for unedited alleles, which is present in both −RT and +RT samples. In addition, FIG. 61C shows qPCR data depicting GFP insertion at 5′ or 3′ junctions at EMX1 locus by two retrons. ncRNA-sgRNA engineered to contain GFP gene insert was transfected to HEK293T cells, together with RT mRNA of each retron, Cas9 mRNA, with/without additional sgRNA. Genomic DNA was analyzed by indicated primer pairs in the table to detect GFP gene insertion at 5′ or 3′ junctions on the edited allele. ΔCt was obtained by subtracting junction Ct value from EMX1 Ct value for relative quantification of GFP insertion at EMX1 locus. With all two retrons, amplicons of both 5′ and 3′ junctions are detectable significantly above background level. Amplicon of 3′ junction is slightly more abundant than that of 5′ junction probably because insertion at 3′ is facilitated by more enriched 5′ end of msDNA from reverse transcription. Thus, two different retrons have been shown to insert a whole gene by retron editing.

In all, these results demonstrated the feasibility of inserting an entire gene by retron-mediated precise editing.

### Example 12. Order of sgRNA and ncRNA in Fused RNA

Next, the order of sgRNA and ncRNA in fusion was addressed. The order might play an important role in ensuring the activity of two enzymes (Cas9 and RT of retron) on one fused substrate. A. ncRNA-sgRNA and B. sgRNA-ncRNA order were compared for precise editing with Eco3, Aco1, and RTX-2042 retron (FIG. 62A). For Eco3 and RTX-2042, sgRNA-ncRNA order allowed significantly higher activity for both precise editing (6- and 3-fold, respectively) and indels (3- and 2-fold respectively) than ncRNA-sgRNA order. The same order provided higher indels for Aco1 (2 fold), suggesting that sgRNA at 5′ of the fusion permits higher Cas9 activity. However, precise editing with Aco1 was comparable between two orders.

When sgRNA spike-in was added to above conditions, the overall activity slightly increased (FIG. 62B). The addition of sgRNA spike-in compensated inferior Cas9 activity with ncRNA-sgRNA (to that with sgRNA-ncRNA), so that precise editing and indels became comparable to the inverse order for all retrons.

The precise editing was dependent on the presence of RT and Cas9 (FIG. 63). When any one of these components is removed, precise editing is undetectable or at background levels with either order of ncRNA and sgRNA fusion (FIG. 63 top graphs). This was consistent with all three retrons. On the bottom graphs, frequency of indels is shown for respective conditions (FIG. 63 bottom graphs).

### Example 13. Separation of ncRNA and sgRNA

The addition of sgRNA spike-in to either order of ncRNA and sgRNA fusion increased precise editing and indels (FIGS. 62A, 62B and FIG. 63). These data suggest that physical fusion of ncRNA and sgRNA of Cas9 nuclease could be a limiting factor for Cas9 activity and consequently precise editing thus separation of ncRNA and sgRNA could be a better strategy for precise editing.

The impact of separating sgRNA from ncRNA on editing was tested in Eco3, Aco1, RTX-2042 and RTX-6943 retron system comparing the editing efficiency between A. ncRNA-sgRNA or B. sgRNA-ncRNA fusion and C. separated ncRNA and sgRNA with or without additional sgRNA (FIG. 64). With Eco3, the separation significantly outperformed (˜5 fold) any order of fusion of ncRNA and sgRNA in precise editing. With no sgRNA added to separated ncRNA, no precise editing was observed, as expected. On the other hand, separated version of Aco1 and RTX-2042 ncRNAs achieved a comparable level of precise editing to fused RNA. Separated ncRNA of RTX-6943 showed lower activity than fused version but the overall activity of this retron is a magnitude lower than others.

These data demonstrates that separation of ncRNA and sgRNA could achieve comparable or even higher precise editing than ncRNA and sgRNA fusion depending on the type of retron.

### Example 14. Retron Mediated Precise Editing Occurs at Another Genomic Locus, AATVS1

All previous gene editing assays were done at EMX1 gene locus. With Eco3, Aco1, RTX2042 and RTX6943 retrons, AAVS1 locus was targeted for precise editing. AAVS1 locus, in intron 1 of PPP1R12C (protein phosphatase 1 regulatory subunit 12C) gene, is known as a “safe harbor”, because its disruption does not have adverse effects on the cell, and robust transcription of locus is permissive for stable expression of an exogenously inserted gene. Within msD stem loop of ncRNA from each retron, 25 bp insertion donor sequence was incorporated and to the 3′ end of engineered ncRNA, sgRNA targeting AAVS1 was fused.

FIG. 65 shows the result of testing four retrons to edit AAVS1 locus. For all four retrons, precise editing was enhanced in the presence of additional sgRNA and ranged from 0.73% to 2.05%. On the right, frequency of indels at respective conditions shares a similar trend of precise editing. Removing either RT, ncRNA-sgRNA or Cas9 mRNA abrogated precise editing showing Retron and Cas9 specificity of editing (FIG. 66), while indels are robust in the bottom graph.

### Example 15. Testing Template Strand Bias for Precise Editing

Cas9 uses different endonuclease domain to make cleavage on either sgRNA targeting or non-targeting strand. Differential release timing of each nuclease domains of Cas9 from DNA substrates after cutting might influence how single strand donor interact with broken DNA and repair pathway (12).

Results of testing donor templates on the opposite strand relative to Cas9 sgRNA targeting strand are shown in FIG. 67. Eco3 and Aco1 retrons in all RNA system were delivered to HEK293T cells by lipofection. The given amount of RT mRNA, Cas9 mRNA, additional sgRNA targeting EMX1 locus, and appropriate ncRNA-sgRNA described underneath the graph were added. All donor templates tested contained 25 bp insert. All ncRNAs used in previous experiments encoded the donor template of the strand targeted by the Cas9 sgRNA and denoted by the letter T. ncRNA that contains reverse complementary sequence of Cas9 targeting strand is denoted as NT. For Eco3, in the presence of additional sgRNA, precise editing was 1.67% when template was on the targeting strand and 0.85% when the template was on the non-targeting strand. For Aco1, in the presence of additional sgRNA, precise editing was 2.96% when template was on the targeting strand and 3.40% when the template was on the non-targeting strand. On the bottom row of graphs, frequency of indels is shown for respective conditions.

At least in EMX1 locus, retron ncRNA encoding either Cas9 targeting or non-targeting strand produced a similar level of precise editing.

### Example 16. Retron Mediated Precise Editing by Cas9 Nickase is Improved by Combining Dual sgRNAs

Retron mediated precise editing with Cas9 nickase was observed but at low efficiency (˜0.1%) in previous study. This is consistent with other studies that a single nick does not efficiently induce gene editing, because the induced single-strand breaks are very efficiently repaired by the base excision repair pathway (22) (23). In contrast, combination of a pair of PAM (protospacer-adjacent motif)-out sgRNAs at proximity with Cas9 nickase led to efficient gene editing (precise edits and indels) (23). This double-nick approach induces a site-specific double strand break leading to target gene indels but off-targets are significantly reduced by 50-1000 fold than Cas9 WT.

Whether double-nick approach supports retron-mediated precise editing, three sgRNAs were selected at the 3′ of last exon of EMX1 (FIG. 70). Combination of sgRNA 1+3, 2+3 and 1+2 were tested with Cas9 D10A nickase in parallel with Cas9 WT using sgRNA1. All RNA was transfected at a fixed amount RT mRNA 100 ng, ncRNA-sgRNA 400 ng, Cas9 variant mRNA 100 ng, and sgRNA 5 ng each. sgRNA 1+3 combination with nickase gave the highest precise editing at 3% efficiency (FIG. 70, top left graph). This efficiency with dual sgRNA is ˜30 fold higher than that achieved using single sgRNA but still lower than Cas9 WT guided by sgRNA1 (13% editing efficiency). The editing was dependent on retron system since the absence of RT abrogated the editing. Precise edits were not proportional but correlated with indel frequency (FIG. 54, bottom left graph).

### Example 17. A Novel Retron R6083 is Capable of Inserting Up to 100 bp in all RNA System

FIG. 71 shows result of testing different template lengths/ncRNA and sgRNA separation or fusion with R6083 retron in all RNA system. All RNA was delivered to HEK293T cells by lipofection. At given amount of RT mRNA and Cas9 mRNA, ncRNA-sgRNA fusion with different template lengths ranging from 25 to 100 bp or ncRNA with 25 bp insertion were added. Precise insertion of 100 bp to EMX1 locus was observed at ˜2% efficiency (FIG. 71 top left graph). Separated ncRNA exhibited lower activity than ncRNA-sgRNA fusion with 25 bp insertion. The precise editing was dependent on the presence of RT and ncRNA as shown in top, right graph. On the bottom row of graphs, frequency of indels is shown for respective conditions.

FIG. 72 shows that R6083 retron was able to insert 25 bp template into AAVS1 locus in all RNA system. With additional sgRNA, 3% of editing efficiency was observed and the activity was completely dependent on the presence of either RT or ncRNA (FIG. 72 left graph).

### Example 18. Cas9-RT Fusion Vs Separated Cas9 and RT

In all previous studies, Cas9 and RT mRNA were delivered separately. Physically fused Cas9−RT could be beneficial for colocalization of two enzymes in cellular compartment but also be harmful by interfering activity of each enzyme. To address this point, Cas9 and Eco3 RT were fused through a SGGSx2-XTEN-SGGSx2 (SEQ ID NO:19932) flexible linker and tested for precise gene editing in parallel with separated format. The same molar concentration of Cas9 (150 ng) and RT mRNA (50 ng) corresponding to 200 ng of Cas9-RT fusion were used in separation and the editing efficiency of fused or separated enzyme were compared for either ncRNA alone (left on left graph of FIG. 73) or ncRNA-sgRNA fusion (right on right graph of FIG. 73). Separated Cas9 and RT achieved higher editing efficiency than when they were fused with both ncRNA (3-fold) and ncRNA-sgRNA (1.5-fold). Higher editing by separated enzymes was more pronounced when ncRNA and sgRNA was separated, suggesting that separated enzymes are more flexible to search for respective targets. Consistent with precise editing, indels from fused enzyme was lower in both ncRNA and ncRNA-sgRNA fusion. These results suggest that Cas9 and RT in fused format could constrain each other even with a flexible linker between.

### Example 19. Retron ncRNA End Protection by Cap and Tail (New Experiment)

5′ and 3′ end of eukaryotic mRNAs are modified by capping and tailing, respectively. Both the cap and the tail protect the transcript from exonucleases and help it get exported from the nucleus and translated on the ribosomes. More recently, long non coding RNAs (>200 nt) are shown to be often capped and tailed (21).

To attempt to increase half-life of retron ncRNA in cells, either cap or tail and both cap and tail were added to ncRNA (FIG. 55 schematic). FIG. 74 shows the results of end protection of ncRNA-sgRNA fusion by cap and tail in 4 component all-RNA system (RT mRNA+Cas9 mRNA+ncRNA+sgRNA) delivered to HEK293T cells by Lipofectamine MessengerMAX. All RNA was transfected at a fixed amount RT mRNA 100 ng, ncRNA-sgRNA 400 ng, Cas9 mRNA 100 ng, and sgRNA 5 ng. ncRNA-gRNA fusion was either capped (+cap −tail) or poly-A tailed (−cap +tail) or both capped and poly-A tailed (+cap +tail). ncRNA without end protection (−cap −tail) produced ˜4.5% precise edits when additional sgRNA was added and the editing was dependent on the presence of RT. While capping alone did not change editing efficiency, tailing alone slightly increased editing and both cap and tail significantly improved editing efficiency about 2-fold (left graph). Indels at all conditions were comparable as shown in the right graph.

These results support that end protection of ncRNA by cap and tail promotes retron-mediated precise editing possibly by enhancing stability of ncRNA within cells.

### Example 20. Circularization of ncRNA

We looked for an alternative way of stabilizing ncRNA. Circular RNA forms a covalently closed continuous loop and this endless form endows extended durability. FIG. 75 describes how circular ncRNA is made. Circularization leveraged on group I self-splicing intron method that has been reported to be more applicable to long RNA circularization and requires only the addition of GTP and Mg2⁺ as cofactors (22). ncRNA is surrounded by self-splicing intron and complementary homology sequences (FIG. 75 step1). External and internal homology sequences bring two introns into proximity (step2). Exogenously added GTP initiates a series of trans-esterification that excises the introns and ligates ncRNA in a circular form (step3).

FIG. 76 summarizes the results of testing modified ncRNAs for precise editing at EMX1 locus by Eco3 all RNA system in HEK293T cells. All RNA was transfected at a fixed amount RT mRNA 100 ng, ncRNA 400 ng, Cas9 mRNA 100 ng, and sgRNA 5 ng. With the addition of MS2 stem loop at 3′ end of ncRNA, the activity nearly doubled at 15% efficiency from unmodified ncRNA (˜8%). Circularization of ncRNA achieved further increased efficiency, reaching at 22%. Circularization of ncRNA was done as described in FIG. 75. Without sgRNA, both precise edit and indels were not detectable as expected. Indels are shown at the right graph at respective conditions of left graph.

### Example 21. RT and its Cognate ncRNA from Same Retron is Required to Support Precise Editing

RT of retron does not require an exogenous primer but instead utilizes a specific branching G residue within a folded secondary structure of template ncRNA to initiate reverse transcription (23). Some early discovered RTs were shown to recognize specifically secondary structure and priming G in their cognate ncRNA for msD synthesis (24). This RT-ncRNA specificity was tested with a newly characterized Aco1 retron in gene editing system in parallel with Eco3, one of early retrons. In consistent with previous studies, Aco1 supported precise editing only when paired with its cognate ncRNA and the activity was abrogated when paired with Eco3 ncRNA (FIG. 77, left graph). The same observation was made with Eco3 retron and Eco3, only with its cognate ncRNA contributed to precise editing. Indels are shown at the right graph at respective conditions of left graph.

### Example 22. Precise Insertion of Exon-Size Sequences

Exon-size long insertion has clinical value to replace a mutation enriched exon causing genetic diseases. For example, exon 8 is the mutation hot spot in Wilson's disease patients with ˜500 mutations that are regionally and ethnically different (25). We tested insertion of different size up to 305 bp with Aco1, Eco3 and a completely novel R1262 retron at EMX1 locus of HEK293T cells. Aco1 achieved precise insertions of 10, 100 and 205 bp at 8, 4, and 1.3% (FIG. 78, left in top graphs). Eco3 made precise insertions of 10 and 205 bp at 12 and 2.3%, respectively (FIG. 78, middle in top graphs). A novel retron R1262 obtained 25, 205, and 305 bp insertion at 20, 15 and 8.8% efficiency (FIG. 78, right in top graphs). The activity of R1262 is notably robust with insignificant drop from 25 to 205 bp size increase, in contrast to Aco1 or Eco3. Furthermore, the ratio of precise edit over indels is the least (1:2) with R1262 (FIG. 78 indels in bottom graphs). Precise editing was abrogated in the absence of RT or sgRNA, indicating that the system is dependent on both retron and site-specific nuclease.

In addition to EMX1 locus, R1262 retron supported 205 bp precise insertion at AAVS1 locus with 11% efficiency (FIG. 79). These data expand the potential of retron-mediated gene editing as a genetic medicine.

### Example 23. Optimization of RT:ncRNA Ratio for >200 bp Long Insertion with R1262 Retron

ncRNA is the source of single strand DNA that serves as a repair template and the more single strand DNA production could induce higher editing. However, ncRNA might shortest live inside cells among RT, Cas9 mRNA, and sgRNA and be a limiting factor in retron based editing system since ncRNA without chemical modification is vulnerable to cellular nucleases and upon reverse transcription, ncRNA is degraded by cellular RNase H (28). Based on these hypotheses, all RNA system is formulated with ncRNA occupying the highest proportion of cocktail and previous studies with Eco3 retron agreed with this point. FIG. 80 shows the results of testing RT: ncRNA ratio for >200 bp long insertion at EMX1 locus with a novel reton R1262. RT: ncRNA ratio was tested at 1:12.5, 1:16.7 and 1:25 molar ratio. 1:16.7 ratio corresponds to 1:4 mass ratio that was used in the rest of studies. With ncRNA (right in the left graph), the best editing was observed at 1:12.5 ratio for both 205 and 305 bp insertion. With ncRNA-sgRNA fusion (left at left graph), all ratio tested generated comparable editing but a weak tendency of increased editing was seen with increased amount of ncRNA-sgRNA.

### Example 24. A Novel Retron R2781 is Capable of Inserting Up to 100 bp in all RNA System

FIG. 105 shows results of testing different template lengths of ncRNA with a novel retron R2781 in an all-RNA system. All RNA was delivered to HEK293T cells by lipofection. At given amount of RT mRNA and Cas9 mRNA, ncRNA with different template length insertion were added. In FIG. 16, R2781 demonstrated 10 bp insertion at EMX1 locus at similar activity as other hits, R1262, R6342S, 6342L in plasmid system. Here, the activity of R2781 for 25 bp, 205 bp and 405 bp insertion at EMX1 locus was evaluated with two different homology arm (HA) length (30 bp for both arms or 49 bp left/65 bp right). Precise insertion of 25 bp with 30 bp homology arm was achieved at 11% efficiency (left graph). The efficiency of same length insertion was reduced to half with longer homology arm (49/65 bp) suggesting that this retron reverse transcriptase might have limited enzymatic processivity. Conversely, the activity inserting 205 or 405 bp significantly dropped. Indels are shown at the right graph at respective conditions of left graph.

### Example 25. Precise Editing with Cas9 Nickase and Retron R6342S

Wild Type (WT) Cas9 nuclease generates double strand break (DSB) that can potentially increase genomic instability by generating larger deletion or chromosome translocation or chromosome loss (32). Single point mutations in either of Cas9 nuclease domains can generate nickases that cut only one strand of targeting site. D10A variant cuts target strand using intact HNH nuclease domain and H840A variant cuts non-target strand using intact RuvC domain. Nicks can initiate homology-directed repair (HDR) and cause less mutagenic end joining than DSBs (13). These features draw considerable interest in using nickases in retron-based precise editing.

Previously, retron based precise insertion of 10 bp was achieved at 0.1% efficiency with Eco3 retron combined with D10A nickase (FIG. 76). Here, DOA wild type and D10A nickase with R221K/N394K mutations previously shown to improve Cas9 nuclease activity (33) was tested with one of lead retrons, R6342S. Precise editing activity by R6342S was observed 3-4 times higher than Eco3 with wild type nickase and further enhanced (7 times higher) with R221K/N394K nickase mutant (FIG. 106).

### Example 26. Retron Meditated Precise Deletion of Genomic DNA

A number of genetic diseases could be corrected by simply deleting aberrant genomic sequences. Dystrophin gene in muscle disease (34) and Col7A1 gene in skin disease (35) and repeat expansion disorders (36) are good examples to delete undesired mutation containing sequences. Here, precise deletion mediated by retron R1262 was tested in an all-RNA system. In FIG. 107, two deletion strategies are depicted in the top panel. Retron ncRNA is designed by juxtaposing left and right homology arms sequences to delete the intervening sequence. Dell is deleting 214 bp upstream of Cas9 cutting site and del2 is deleting 248 bp downstream of Cas9 cutting site at EMX1 locus. Retron mediated deletion was compared with direct delivery of increasing amounts of single strand DNA (ssDNA) donors (150 and 300 ng). In bottom panel, left graph shows that R1262 was able to delete 248 bp (del2) from EMX1 locus at similar activity as ssDNA donor. Indels are shown on the right graph at respective conditions of left graph. Of note, unintended indel activity by retron-mediated deletion was ˜6 fold lower than ssDNA donor mediated deletion. Deletion activity of 214 bp (dell) by R1262 was not detected.

This data suggests that retron could be used for precise deletion of genomic DNA at similar activity as ssDNA donor but with lower unintended activity.

### Example 27. NHEJ-Based Insertion of Retron-Derived Double Strand DNA at Target Site

The current approach for retron mediated gene editing is dependent on homology dependent repair (HDR) mechanism, which is only active in dividing cells. Most retrons generate single strand DNA (ssDNA), which can be used for HDR template. In order to apply retron mediated editing to non-dividing cells where HDR is not active (e.g., in non-dividing cells), alternative approaches could be considered. One such alternative approach is to utilize nonhomologous DNA end-joining (NHEJ) to integrate double strand DNA (dsDNA) at a target cut site. NHEJ based integration of dsODN/dsDNA has been demonstrated various times in literature and has worked in cells and in vivo. This approach can be used for small insertions as well as large insertions such as exon replacement and safe harbour knock-in.

A dsDNA template may be formed from retrons by employing two ncRNAs of any retron whose encoded RT ssDNA products are complementary to each other and which form a duplex to become dsDNA (FIG. 108, schematics in left). These dsDNAs contain msR and msD spacer sequences at both extremes that do not hybridize each other and could be inhibitory for end-joining. The inclusion of sgRNA recognition sequences (same as genomic target sequence) in tandem at both ends of insert leads to blunt-ended double strand DNA and one direction of insertion is more stably installed than the other since it prevents from recutting by Cas9. Insertion in the other direction allows Cas9 recutting thus the edited sequence is less stable. Using this strategy, R1262 retron with two complementary ncRNAs inserted ˜120 bp dsDNA on EMX1 locus at ˜1% efficiency, higher when insert sequence was flanked with two sgRNA sequences in tandem (FIG. 108, top right graph). Basal insertion activity of R6342 without tandem sgRNA sequences was slightly higher than that of R1262. Indels are shown at the right, bottom graph at respective conditions of top graph. This data demonstrates that retron-derived dsDNA could directly integrate at the target site by an NHEJ mechanism and does not require homology with the site of integration.

### Example 28. Retron RNAs do not Trigger Overt Immune Response

Potential immunogenicity of retron mediated editing was tested in an all-RNA system using human peripheral blood mononuclear cells (hPBMCs). PBMCs contain both innate and adaptive immune cells and these cells are equipped with sensors to detect foreign nucleic acids. RNA quality and the presence of 5′ triphosphate of RNA generated by in vitro transcription could activate immune sensors. In addition, single strand DNAs made by Retron RT using ncRNA as template could potentially trigger immune response. To reduce immune reaction, uridine in RT mRNA was replaced by less immunogenic m1Ψ (37) and 5′ triphosphate of ncRNA is capped with m7G. These modified versions of RNA were by themselves or in combination, electroporated to hPBMCs along with GFP mRNA without base modification (TriLink) as a control. After overnight culture, the supernatant was analyzed for cytokine production. Among 25 cytokines and chemokines examined, those above detection limit are shown in FIG. 109. Type I interferon, a hallmark of immune response to foreign nucleic acids was not detected in any retron RNA transfected cells and some inflammatory cytokines/chemokines were detected at low level in retron RNA transfected cells, but at much lower level than GFP mRNA control except RT mRNA without U modification. Of note, ncRNA was not modified for U as GFP mRNA control and was electroporated four times more than RT mRNA or GFP mRNA reflecting the condition used for gene editing. In spite of this similarity and difference, ncRNA transfected cells produced much less cytokines/chemokines than GFP mRNA control.

### Example 29. Evidence of Retron-Mediated Precise Editing in Primary Cells

First, bone marrow derived CD34+human stem cells (HSCs) were used to evaluate the performance of retron-mediated gene editing in primary cells. HSCs are long live and capable of generating entire hematopoietic system so that HSC transplant has been used for decades to treat hematopoietic disorders such as genetic diseases or leukemias. While transplant therapy has been supportive for patients, it lacks definitive curative outcome. In contrast, direct correction of mutations present in the genome of patient HSCs holds the potential for a curative effect. The confirmation of this notion will eventually emerge through HSC gene editing therapy in clinical setting.

In FIG. 110, results of testing retron-mediated gene editing in human stem progenitor cells in an all-RNA system are shown. Frozen HSCs were thawed to expand for three days in the presence of hSCF, hFLT3-L, hTPO cytokines to prevent differentiation. A total of 3 or 5 μg of Cas9 mRNA, guide RNA (gRNA), R6342 RT mRNA and ncRNA were mixed at mass ratio indicated in each label at the left graph, and electroporated to HSCs. After another three-day culture, genomic DNA was extracted and sequenced to measure editing frequency. With total of 5 microgram of RNA, divided at ratio of 1:0.3:4:1=Cas9:gRNA:ncRNA:RT, precise insertion of 25 bp at AAVS1 locus was observed at 0.1% of frequency (left graph). Indels in their respective condition are shown at the right graph.

Second, T-cells are the cells of the immune system responsible for seeking out and destroying unhealthy cells. These unhealthy cells are typically cells infected with harmful pathogen, but some T-cells can also naturally recognize and kill cancer cells. Genetic engineering of T-cell genome enhances such T-cell function and immunotherapies with enhanced T-cell function have provided remarkable clinical success to treat B cell acute lymphoblastic leukemia (38). Based on these successes, a wide range of clinical developments against cancer, infectious diseases, and autoimmune diseases have been leveraging on T-cell genome engineering.

In FIG. 111, results of testing retron-mediated gene editing in human T-cells in all-RNA system are shown. Frozen human pan T-cells from peripheral blood were thawed and activated for two days in the presence of anti-CD3/anti-CD28 conjugated magnetic beads and IL-2 cytokine. After the culture, anti-CD3/anti-CD28 beads were removed from cells by magnet. A total of 3 or 5 μg of Cas9 mRNA, guide RNA (gRNA), R6342 RT mRNA and ncRNA were mixed at mass ratio indicated in each label at the left graph, and electroporated to T-cells by either Neon or Lonza electroporator. After an additional three-day culture, genomic DNA was extracted and sequenced to measure editing frequency. With total of 3 μg of RNA, divided at 1:0.3:3:1=Cas9:gRNA:ncRNA:RT, precise insertion of 25 bp at AAVS1 locus was observed up to 1.7% of frequency using Neon machine (left graph). Indels in their respective condition are shown in the right graph.

### Example 30. Retron-Based Gene Editing In Vivo Using LNP

In certain embodiments, retron-mediated gene editing can be achieved with an-all RNA system without requiring a viral DNA donor or exogenous DNA donor since retrons are able to produce donor DNA from a ncRNA template inside cells. As proposed herein, this feature is particularly apt for lipid nanoparticle (LNP)-based platform. Following the methods described herein, the performance of retron-based gene editing in vivo is investigated. Retron-based gene editing system could be formulated in one or more LNPs.

An exemplary and non-limiting LNP formulation system may be prepared for in vivo delivery of a retron editing system.

### LNP Formulation for In Vivo Delivery of Retron-Based Editing System

Ionizable lipids, phospholipids, cholesterol, and PEG-lipids are dissolved in pure ethanol at specified molar ratios (exemplary formulations shown below as Formulation Molar Ratios A, B, C, and D), with a total lipid concentration of about 7.2 mM. A polynucleotide solution (exemplary concentration of 0.067 mg/mL) is prepared using acidic buffer (pH 4.0-5.0) containing a gene editing system, such as a 1:0.3:4:1 ratio of a Cas9 mRNA/gRNA/ncRNA/RT. The nucleotide and lipid solutions are mixed at a 3:1 volume ratio using a NanoAssemblr microfluidic system at a 12 mL/min total flow rate resulting in rapid mixing and self-assembly of LNPs. Formulations are further dialyzed against PBS (pH 7.4) overnight at 4° C., concentrated using centrifugal filtration and filtered (0.2 μm pore size). The particle size and polydispersity index (PDI) of formulations was measured by dynamic light scattering (DLS) using a Zetasizer Ultra (Malvern Panalytical). RNA encapsulation efficiency (EE %) was determined by Ribogreen assay.

### Formulation Molar Ratio A:

| Ionizable Lipid | DSPC | Cholesterol | DMG PEG-2k |
|---|---|---|---|
| 48.5 | 10 | 39 | 2.5 |

### Formulation Molar Ratio B:

| Ionizable Lipid | DSPC | Cholesterol | DMG PEG-2k |
|---|---|---|---|
| 55 | 10 | 32.5 | 2.5 |

### Formulation Molar Ratio C:

| Ionizable Lipid | DSPC | Cholesterol | DMPE PEG-2k |
|---|---|---|---|
| 55 | 10 | 32.5 | 2.5 |

### Formulation Molar Ratio D:

| Ionizable Lipid | DSPC | Cholesterol | DSPE PEG-2k |
|---|---|---|---|
| 48.5 | 10 | 40 | 1.5 |

### In Vivo Delivery Protocol of LNP-Based Retron Editing System

CD-1 female mice, ranging from new-born to adult age (6-10 weeks) are used in each study. LNPs are dosed via the lateral tail vein in a volume of approximately 5 mL per kilogram body weight. The animals are periodically observed for adverse effects for at least 24 hours post dose. Mice are dosed at 2 mpk. Each formulation is dosed in 5 animals. Animals are euthanized at 7 days by exsanguination via cardiac puncture under isoflurane anesthesia. On-target/off-target tissues are collected from each animal for DNA extraction and analysis. Cohorts of mice are measured for editing by Next-Generation Sequencing (NGS). Overall health and well-being of animals is recorded to determine whether delivery of gene editing payloads resulted in deleterious off-target editing.

### Example 31. Retron ncRNA Sequence Optimization Via High-Throughput Screening of ncRNA Variant Library

Retrons are native to prokaryotes and hence, the ncRNA sequences can be further engineered to accommodate their application and maximize efficiency in human cells.

To understand and optimize the editing efficiency by retron ncRNA in human cells, a pooled ncRNA library with 191 variants (sequences provided in Table 31A below) plus 2 controls (a positive and negative control) of different lengths and sequences in several key elements (such as, but not limited to, modifications to the lengths of the a1:a2 stem, msR stem-loop) was developed. Each of these variants is associated with unique barcodes designed in the donor region, and the variant library is synthesized as a pooled oligo library. The synthesized oligo library is cloned into a pooled DNA library, from which pooled ncRNAs are produced by in vitro transcription. RNA library is then transfected to the 293T cell line and we harvest cells at different timepoints post-transfection. Next-generation sequencing is then applied for the unique barcodes to measure the abundance of each RNA variant, production of the ssDNA donor, and the precise insertion at the edited locus. Through comparing these data sets, the stability and efficacy of each ncRNA variant can be scored. From the scoring insight, enhanced ncRNA can be optimized/engineered by integrating the most effective modified features.

### Methods:

191 ncRNA variants are designed with variation introduced to the following ncRNA elements: (1) al a2; (2) msD stem; (3) msR stem; (4) msR loop; (5) termination sequence (within msR spacer region); (6) msD, msR spacer deletions (minimal variants). In addition, one positive control (WT R6342S ncRNA) and a negative control with disrupted reverse transcription priming sites. Each of these variants is associated with several unique barcodes (e.g., “ACCTATCATTCANNNNNNNN”) designed in the donor region which targets human EMX1 gene. The variant library is synthesized as a pooled oligo library. The synthesized oligo library is assembled into a pooled DNA library, from which pooled ncRNAs are produced by in vitro transcription. The ncRNA variant sequences and the control sequences are provided in Table 31A. A representative library ncRNA construct is depicted in FIG. 112. The types of variations in the library is depicted in FIG. 113.

TABLE 31A
ncRNA variant sequences and barcodes

| | | SEQ | | SEQ | |
|---|---|---|---|---|---|
| | DNA sequence (changed region are labelled with | ID | | ID | |
| Variants | lower case) | NO: | Barcodes | NO: | Type |
| Positive | CATAGATTTCTTGGCCTTTATGCTGTGGTGTTGC | | ACCTAT | | Positive |
| control | GCCACGGTGGAGATTTGTCAAATACACATCATT | | CATTCA | | control |
| (WT) | AGGTTGCGACAAACGGCAGAAGCTGGAGGAGG | | NNNNN | | |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Negative | CATAGATTTCTTatCCTTTATGCTGTGGTGTTGCG | | ACCTAT | | Negative |
| control | CCACGGTGGAGATTTGTCAAATACACATCATTA | | CATTCA | | control |
| | GGTTGCGACAAACGGCAGAAGCTGGAGGAGGA | | NNNNN | | |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0001 | tataaGCCTTTATGCTGTGGTGTTGCGCCACGGTG | 19543 | ACCTAT | 19735 | ala2 |
| | GAGATTTGTCAAATACACATCATTAGGTTGCGA | | CATTCA | | variant |
| | CAAACGGCAGAAGCTGGAGGAGGAAGGGCCTG | | NNNNN | | |
| | AGTCCGAGCAGAAGAACCTGCAGGACCTATCA | | NNN | | |
| | TTCANNNNNNNNAAAGTTCTCCCATCACATCAA | | | | |
| | CCGGTGGCGCATTGCCACGAAGCAGGCCAATG | | | | |
| | GGGAGGACATCGATGTCACACAACCAAATATA | | | | |
| | AGAATTGTTAGttata | | | | |
| Var0002 | tgataGCCTTTATGCTGTGGTGTTGCGCCACGGTG | 19544 | ACCTAT | 19736 | ala2 |
| | GAGATTTGTCAAATACACATCATTAGGTTGCGA | | CATTCA | | variant |
| | CAAACGGCAGAAGCTGGAGGAGGAAGGGCCTG | | NNNNN | | |
| | AGTCCGAGCAGAAGAACCTGCAGGACCTATCA | | NNN | | |
| | TTCANNNNNNNNAAAGTTCTCCCATCACATCAA | | | | |
| | CCGGTGGCGCATTGCCACGAAGCAGGCCAATG | | | | |
| | GGGAGGACATCGATGTCACACAACCAAATATA | | | | |
| | AGAATTGTTAGtatca | | | | |
| Var0003 | tctacGCCTTTATGCTGTGGTGTTGCGCCACGGTG | 19545 | ACCTAT | 19737 | ala2 |
| | GAGATTTGTCAAATACACATCATTAGGTTGCGA | | CATTCA | | variant |
| | CAAACGGCAGAAGCTGGAGGAGGAAGGGCCTG | | NNNNN | | |
| | AGTCCGAGCAGAAGAACCTGCAGGACCTATCA | | NNN | | |
| | TTCANNNNNNNNAAAGTTCTCCCATCACATCAA | | | | |
| | CCGGTGGCGCATTGCCACGAAGCAGGCCAATG | | | | |
| | GGGAGGACATCGATGTCACACAACCAAATATA | | | | |
| | AGAATTGTTAGgtaga | | | | |
| Var0004 | gatccGCCTTTATGCTGTGGTGTTGCGCCACGGTG | 19546 | ACCTAT | 19738 | ala2 |
| | GAGATTTGTCAAATACACATCATTAGGTTGCGA | | CATTCA | | variant |
| | CAAACGGCAGAAGCTGGAGGAGGAAGGGCCTG | | NNNNN | | |
| | AGTCCGAGCAGAAGAACCTGCAGGACCTATCA | | NNN | | |
| | TTCANNNNNNNNAAAGTTCTCCCATCACATCAA | | | | |
| | CCGGTGGCGCATTGCCACGAAGCAGGCCAATG | | | | |
| | GGGAGGACATCGATGTCACACAACCAAATATA | | | | |
| | AGAATTGTTAGggatc | | | | |
| Var0005 | acgcgGCCTTTATGCTGTGGTGTTGCGCCACGGTG | 19547 | ACCTAT | 19739 | ala2 |
| | GAGATTTGTCAAATACACATCATTAGGTTGCGA | | CATTCA | | variant |
| | CAAACGGCAGAAGCTGGAGGAGGAAGGGCCTG | | NNNNN | | |
| | AGTCCGAGCAGAAGAACCTGCAGGACCTATCA | | NNN | | |
| | TTCANNNNNNNNAAAGTTCTCCCATCACATCAA | | | | |
| | CCGGTGGCGCATTGCCACGAAGCAGGCCAATG | | | | |
| | GGGAGGACATCGATGTCACACAACCAAATATA | | | | |
| | AGAATTGTTAGcgcgt | | | | |
| Var0006 | cgcgcGCCTTTATGCTGTGGTGTTGCGCCACGGTG | 19548 | ACCTAT | 19740 | ala2 |
| | GAGATTTGTCAAATACACATCATTAGGTTGCGA | | CATTCA | | variant |
| | CAAACGGCAGAAGCTGGAGGAGGAAGGGCCTG | | NNNNN | | |
| | AGTCCGAGCAGAAGAACCTGCAGGACCTATCA | | NNN | | |
| | TTCANNNNNNNNAAAGTTCTCCCATCACATCAA | | | | |
| | CCGGTGGCGCATTGCCACGAAGCAGGCCAATG | | | | |
| | GGGAGGACATCGATGTCACACAACCAAATATA | | | | |
| | AGAATTGTTAGgcgcg | | | | |
| Var0007 | tattaaatttGCCTTTATGCTGTGGTGTTGCGCCACGG | 19549 | ACCTAT | 19741 | ala2 |
| | TGGAGATTTGTCAAATACACATCATTAGGTTGC | | CATTCA | | variant |
| | GACAAACGGCAGAAGCTGGAGGAGGAAGGGCC | | NNNNN | | |
| | TGAGTCCGAGCAGAAGAACCTGCAGGACCTAT | | NNN | | |
| | CATTCANNNNNNNNAAAGTTCTCCCATCACATC | | | | |
| | AACCGGTGGCGCATTGCCACGAAGCAGGCCAA | | | | |
| | TGGGGAGGACATCGATGTCACACAACCAAATA | | | | |
| | TAAGAATTGTTAGaaatttaata | | | | |
| Var0008 | tataatacgaGCCTTTATGCTGTGGTGTTGCGCCACG | 19550 | ACCTAT | 19742 | ala2 |
| | GTGGAGATTTGTCAAATACACATCATTAGGTTG | | CATTCA | | variant |
| | CGACAAACGGCAGAAGCTGGAGGAGGAAGGGC | | NNNNN | | |
| | CTGAGTCCGAGCAGAAGAACCTGCAGGACCTA | | NNN | | |
| | TCATTCANNNNNNNNAAAGTTCTCCCATCACAT | | | | |
| | CAACCGGTGGCGCATTGCCACGAAGCAGGCCA | | | | |
| | ATGGGGAGGACATCGATGTCACACAACCAAAT | | | | |
| | ATAAGAATTGTTAGtcgtattata | | | | |
| Var0009 | ttctgccaatGCCTTTATGCTGTGGTGTTGCGCCACGG | 19551 | ACCTAT | 19743 | ala2 |
| | TGGAGATTTGTCAAATACACATCATTAGGTTGC | | CATTCA | | variant |
| | GACAAACGGCAGAAGCTGGAGGAGGAAGGGCC | | NNNNN | | |
| | TGAGTCCGAGCAGAAGAACCTGCAGGACCTAT | | NNN | | |
| | CATTCANNNNNNNNAAAGTTCTCCCATCACATC | | | | |
| | AACCGGTGGCGCATTGCCACGAAGCAGGCCAA | | | | |
| | TGGGGAGGACATCGATGTCACACAACCAAATA | | | | |
| | TAAGAATTGTTAGattggcagaa | | | | |
| Var0010 | tctcctcgagGCCTTTATGCTGTGGTGTTGCGCCACG | 19552 | ACCTAT | 19744 | ala2 |
| | GTGGAGATTTGTCAAATACACATCATTAGGTTG | | CATTCA | | variant |
| | CGACAAACGGCAGAAGCTGGAGGAGGAAGGGC | | NNNNN | | |
| | CTGAGTCCGAGCAGAAGAACCTGCAGGACCTA | | NNN | | |
| | TCATTCANNNNNNNNAAAGTTCTCCCATCACAT | | | | |
| | CAACCGGTGGCGCATTGCCACGAAGCAGGCCA | | | | |
| | ATGGGGAGGACATCGATGTCACACAACCAAAT | | | | |
| | ATAAGAATTGTTAGctcgaggaga | | | | |
| Var0011 | ccgggttcgcGCCTTTATGCTGTGGTGTTGCGCCACG | 19553 | ACCTAT | 19745 | ala2 |
| | GTGGAGATTTGTCAAATACACATCATTAGGTTG | | CATTCA | | variant |
| | CGACAAACGGCAGAAGCTGGAGGAGGAAGGGC | | NNNNN | | |
| | CTGAGTCCGAGCAGAAGAACCTGCAGGACCTA | | NNN | | |
| | TCATTCANNNNNNNNAAAGTTCTCCCATCACAT | | | | |
| | CAACCGGTGGCGCATTGCCACGAAGCAGGCCA | | | | |
| | ATGGGGAGGACATCGATGTCACACAACCAAAT | | | | |
| | ATAAGAATTGTTAGgcgaacccgg | | | | |
| Var0012 | ggccgggcccGCCTTTATGCTGTGGTGTTGCGCCAC | 19554 | ACCTAT | 19746 | ala2 |
| | GGTGGAGATTTGTCAAATACACATCATTAGGTT | | CATTCA | | variant |
| | GCGACAAACGGCAGAAGCTGGAGGAGGAAGGG | | NNNNN | | |
| | CCTGAGTCCGAGCAGAAGAACCTGCAGGACCT | | NNN | | |
| | ATCATTCANNNNNNNNAAAGTTCTCCCATCACA | | | | |
| | TCAACCGGTGGCGCATTGCCACGAAGCAGGCC | | | | |
| | AATGGGGAGGACATCGATGTCACACAACCAAA | | | | |
| | TATAAGAATTGTTAGgggcccggcc | | | | |
| Var0013 | aaatttaattataaaGCCTTTATGCTGTGGTGTTGCGCCA | 19555 | ACCTAT | 19747 | ala2 |
| | CGGTGGAGATTTGTCAAATACACATCATTAGGT | | CATTCA | | variant |
| | TGCGACAAACGGCAGAAGCTGGAGGAGGAAGG | | NNNNN | | |
| | GCCTGAGTCCGAGCAGAAGAACCTGCAGGACC | | NNN | | |
| | TATCATTCANNNNNNNNAAAGTTCTCCCATCAC | | | | |
| | ATCAACCGGTGGCGCATTGCCACGAAGCAGGC | | | | |
| | CAATGGGGAGGACATCGATGTCACACAACCAA | | | | |
| | ATATAAGAATTGTTAGtttataattaaattt | | | | |
| Var0014 | atattctattacttgGCCTTTATGCTGTGGTGTTGCGCCA | 19556 | ACCTAT | 19748 | ala2 |
| | CGGTGGAGATTTGTCAAATACACATCATTAGGT | | CATTCA | | variant |
| | TGCGACAAACGGCAGAAGCTGGAGGAGGAAGG | | NNNNN | | |
| | GCCTGAGTCCGAGCAGAAGAACCTGCAGGACC | | NNN | | |
| | TATCATTCANNNNNNNNAAAGTTCTCCCATCAC | | | | |
| | ATCAACCGGTGGCGCATTGCCACGAAGCAGGC | | | | |
| | CAATGGGGAGGACATCGATGTCACACAACCAA | | | | |
| | ATATAAGAATTGTTAGcaagtaatagaatat | | | | |
| Var0015 | gcgtatagtaatctgGCCTTTATGCTGTGGTGTTGCGCC | 19557 | ACCTAT | 19749 | ala2 |
| | ACGGTGGAGATTTGTCAAATACACATCATTAGG | | CATTCA | | variant |
| | TTGCGACAAACGGCAGAAGCTGGAGGAGGAAG | | NNNNN | | |
| | GGCCTGAGTCCGAGCAGAAGAACCTGCAGGAC | | NNN | | |
| | CTATCATTCANNNNNNNNAAAGTTCTCCCATCA | | | | |
| | CATCAACCGGTGGCGCATTGCCACGAAGCAGG | | | | |
| | CCAATGGGGAGGACATCGATGTCACACAACCA | | | | |
| | AATATAAGAATTGTTAGcagattactatacgc | | | | |
| Var0016 | aacgcgaaacgctggGCCTTTATGCTGTGGTGTTGCGC | 19558 | ACCTAT | 19750 | ala2 |
| | CACGGTGGAGATTTGTCAAATACACATCATTAG | | CATTCA | | variant |
| | GTTGCGACAAACGGCAGAAGCTGGAGGAGGAA | | NNNNN | | |
| | GGGCCTGAGTCCGAGCAGAAGAACCTGCAGGA | | NNN | | |
| | CCTATCATTCANNNNNNNNAAAGTTCTCCCATC | | | | |
| | ACATCAACCGGTGGCGCATTGCCACGAAGCAG | | | | |
| | GCCAATGGGGAGGACATCGATGTCACACAACC | | | | |
| | AAATATAAGAATTGTTAGccagcgtttcgcgtt | | | | |
| Var0017 | gaggcgggtgccgcaGCCTTTATGCTGTGGTGTTGCGC | 19559 | ACCTAT | 19751 | ala2 |
| | CACGGTGGAGATTTGTCAAATACACATCATTAG | | CATTCA | | variant |
| | GTTGCGACAAACGGCAGAAGCTGGAGGAGGAA | | NNNNN | | |
| | GGGCCTGAGTCCGAGCAGAAGAACCTGCAGGA | | NNN | | |
| | CCTATCATTCANNNNNNNNAAAGTTCTCCCATC | | | | |
| | ACATCAACCGGTGGCGCATTGCCACGAAGCAG | | | | |
| | GCCAATGGGGAGGACATCGATGTCACACAACC | | | | |
| | AAATATAAGAATTGTTAGtgcggcacccgcctc | | | | |
| Var0018 | gcggccgcgggcgggGCCTTTATGCTGTGGTGTTGCGC | 19560 | ACCTAT | 19752 | ala2 |
| | CACGGTGGAGATTTGTCAAATACACATCATTAG | | CATTCA | | variant |
| | GTTGCGACAAACGGCAGAAGCTGGAGGAGGAA | | NNNNN | | |
| | GGGCCTGAGTCCGAGCAGAAGAACCTGCAGGA | | NNN | | |
| | CCTATCATTCANNNNNNNNAAAGTTCTCCCATC | | | | |
| | ACATCAACCGGTGGCGCATTGCCACGAAGCAG | | | | |
| | GCCAATGGGGAGGACATCGATGTCACACAACC | | | | |
| | AAATATAAGAATTGTTAGcccgcccgcggccgc | | | | |
| Var0019 | aatattatattaaatattatGCCTTTATGCTGTGGTGTTGCGC | 19561 | ACCTAT | 19753 | ala2 |
| | CACGGTGGAGATTTGTCAAATACACATCATTAG | | CATTCA | | variant |
| | GTTGCGACAAACGGCAGAAGCTGGAGGAGGAA | | NNNNN | | |
| | GGGCCTGAGTCCGAGCAGAAGAACCTGCAGGA | | NNN | | |
| | CCTATCATTCANNNNNNNNAAAGTTCTCCCATC | | | | |
| | ACATCAACCGGTGGCGCATTGCCACGAAGCAG | | | | |
| | GCCAATGGGGAGGACATCGATGTCACACAACC | | | | |
| | AAATATAAGAATTGTTAGataatatttaatataatatt | | | | |
| Var0020 | gaacaaactaaatataaatcGCCTTTATGCTGTGGTGTTGC | 19562 | ACCTAT | 19754 | ala2 |
| | GCCACGGTGGAGATTTGTCAAATACACATCATT | | CATTCA | | variant |
| | AGGTTGCGACAAACGGCAGAAGCTGGAGGAGG | | NNNNN | | |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CAAATATAAGAATTGTTAGgatttatatttagtttgttc | | | | |
| Var0021 | cgatttctaagacttcggtaGCCTTTATGCTGTGGTGTTGCG | 19563 | ACCTAT | 19755 | ala2 |
| | CCACGGTGGAGATTTGTCAAATACACATCATTA | | CATTCA | | variant |
| | GGTTGCGACAAACGGCAGAAGCTGGAGGAGGA | | NNNNN | | |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGtaccgaagtcttagaaatcg | | | | |
| Var0022 | gtcagggaactccaggaggaGCCTTTATGCTGTGGTGTTG | 19564 | ACCTAT | 19756 | ala2 |
| | CGCCACGGTGGAGATTTGTCAAATACACATCAT | | CATTCA | | variant |
| | TAGGTTGCGACAAACGGCAGAAGCTGGAGGAG | | NNNNN | | |
| | GAAGGGCCTGAGTCCGAGCAGAAGAACCTGCA | | NNN | | |
| | GGACCTATCATTCANNNNNNNNAAAGTTCTCCC | | | | |
| | ATCACATCAACCGGTGGCGCATTGCCACGAAGC | | | | |
| | AGGCCAATGGGGAGGACATCGATGTCACACAA | | | | |
| | CCAAATATAAGAATTGTTAGtcctcctggagttccctgac | | | | |
| Var0023 | cgtgggccgcgcttgagccgGCCTTTATGCTGTGGTGTTG | 19565 | ACCTAT | 19757 | ala2 |
| | CGCCACGGTGGAGATTTGTCAAATACACATCAT | | CATTCA | | variant |
| | TAGGTTGCGACAAACGGCAGAAGCTGGAGGAG | | NNNNN | | |
| | GAAGGGCCTGAGTCCGAGCAGAAGAACCTGCA | | NNN | | |
| | GGACCTATCATTCANNNNNNNNAAAGTTCTCCC | | | | |
| | ATCACATCAACCGGTGGCGCATTGCCACGAAGC | | | | |
| | AGGCCAATGGGGAGGACATCGATGTCACACAA | | | | |
| | CCAAATATAAGAATTGTTAGcggctcaagcgcggcccacg | | | | |
| Var0024 | cggccgccggcgccggcgcgGCCTTTATGCTGTGGTGTTG | 19566 | ACCTAT | 19758 | ala2 |
| | CGCCACGGTGGAGATTTGTCAAATACACATCAT | | CATTCA | | variant |
| | TAGGTTGCGACAAACGGCAGAAGCTGGAGGAG | | NNNNN | | |
| | GAAGGGCCTGAGTCCGAGCAGAAGAACCTGCA | | NNN | | |
| | GGACCTATCATTCANNNNNNNNAAAGTTCTCCC | | | | |
| | ATCACATCAACCGGTGGCGCATTGCCACGAAGC | | | | |
| | AGGCCAATGGGGAGGACATCGATGTCACACAA | | | | |
| | CCAAATATAAGAATTGTTAGcgcgccggcgccggcggcc | | | | |
| | g | | | | |
| Var0025 | attaatatataaatttaattatataGCCTTTATGCTGTGGTGTTG | 19567 | ACCTAT | 19759 | ala2 |
| | CGCCACGGTGGAGATTTGTCAAATACACATCAT | | CATTCA | | variant |
| | TAGGTTGCGACAAACGGCAGAAGCTGGAGGAG | | NNNNN | | |
| | GAAGGGCCTGAGTCCGAGCAGAAGAACCTGCA | | NNN | | |
| | GGACCTATCATTCANNNNNNNNAAAGTTCTCCC | | | | |
| | ATCACATCAACCGGTGGCGCATTGCCACGAAGC | | | | |
| | AGGCCAATGGGGAGGACATCGATGTCACACAA | | | | |
| | CCAAATATAAGAATTGTTAGtatataattaaatttatatattaat | | | | |
| Var0026 | gttatactaataagaattatctgaaGCCTTTATGCTGTGGTGTT | 19568 | ACCTAT | 19760 | ala2 |
| | GCGCCACGGTGGAGATTTGTCAAATACACATCA | | CATTCA | | variant |
| | TTAGGTTGCGACAAACGGCAGAAGCTGGAGGA | | NNNNN | | |
| | GGAAGGGCCTGAGTCCGAGCAGAAGAACCTGC | | NNN | | |
| | AGGACCTATCATTCANNNNNNNNAAAGTTCTCC | | | | |
| | CATCACATCAACCGGTGGCGCATTGCCACGAAG | | | | |
| | CAGGCCAATGGGGAGGACATCGATGTCACACA | | | | |
| | ACCAAATATAAGAATTGTTAGttcagataattcttattagtat | | | | |
| | aac | | | | |
| Var0027 | ttaagatagaggcacttctagtgagGCCTTTATGCTGTGGTGT | 19569 | ACCTAT | 19761 | ala2 |
| | TGCGCCACGGTGGAGATTTGTCAAATACACATC | | CATTCA | | variant |
| | ATTAGGTTGCGACAAACGGCAGAAGCTGGAGG | | NNNNN | | |
| | AGGAAGGGCCTGAGTCCGAGCAGAAGAACCTG | | NNN | | |
| | CAGGACCTATCATTCANNNNNNNNAAAGTTCTC | | | | |
| | CCATCACATCAACCGGTGGCGCATTGCCACGAA | | | | |
| | GCAGGCCAATGGGGAGGACATCGATGTCACAC | | | | |
| | AACCAAATATAAGAATTGTTAGctcactagaagtgcctct | | | | |
| | atcttaa | | | | |
| Var0028 | caggcagcacgcacacagccgaaatGCCTTTATGCTGTGGTG | 19570 | ACCTAT | 19762 | ala2 |
| | TTGCGCCACGGTGGAGATTTGTCAAATACACAT | | CATTCA | | variant |
| | CATTAGGTTGCGACAAACGGCAGAAGCTGGAG | | NNNNN | | |
| | GAGGAAGGGCCTGAGTCCGAGCAGAAGAACCT | | NNN | | |
| | GCAGGACCTATCATTCANNNNNNNNAAAGTTCT | | | | |
| | CCCATCACATCAACCGGTGGCGCATTGCCACGA | | | | |
| | AGCAGGCCAATGGGGAGGACATCGATGTCACA | | | | |
| | CAACCAAATATAAGAATTGTTAGatttcggctgtgtgcgt | | | | |
| | gctgcctg | | | | |
| Var0029 | caccactggcgccgccagcaggcggGCCTTTATGCTGTGGTG | 19571 | ACCTAT | 19763 | ala2 |
| | TTGCGCCACGGTGGAGATTTGTCAAATACACAT | | CATTCA | | variant |
| | CATTAGGTTGCGACAAACGGCAGAAGCTGGAG | | NNNNN | | |
| | GAGGAAGGGCCTGAGTCCGAGCAGAAGAACCT | | NNN | | |
| | GCAGGACCTATCATTCANNNNNNNNAAAGTTCT | | | | |
| | CCCATCACATCAACCGGTGGCGCATTGCCACGA | | | | |
| | AGCAGGCCAATGGGGAGGACATCGATGTCACA | | | | |
| | CAACCAAATATAAGAATTGTTAGccgcctgctggcggc | | | | |
| | gccagtggtg | | | | |
| Var0030 | cgcggccgcgccgggcgcgcgcccgGCCTTTATGCTGTGGT | 19572 | ACCTAT | 19764 | ala2 |
| | GTTGCGCCACGGTGGAGATTTGTCAAATACACA | | CATTCA | | variant |
| | TCATTAGGTTGCGACAAACGGCAGAAGCTGGA | | NNNNN | | |
| | GGAGGAAGGGCCTGAGTCCGAGCAGAAGAACC | | NNN | | |
| | TGCAGGACCTATCATTCANNNNNNNNAAAGTTC | | | | |
| | TCCCATCACATCAACCGGTGGCGCATTGCCACG | | | | |
| | AAGCAGGCCAATGGGGAGGACATCGATGTCAC | | | | |
| | ACAACCAAATATAAGAATTGTTAGcgggcgcgcgccc | | | | |
| | ggcgcggccgcg | | | | |
| Var0031 | aataattattatataataatatattaataaGCCTTTATGCTGTGGTG | 19573 | ACCTAT | 19765 | ala2 |
| | TTGCGCCACGGTGGAGATTTGTCAAATACACAT | | CATTCA | | variant |
| | CATTAGGTTGCGACAAACGGCAGAAGCTGGAG | | NNNNN | | |
| | GAGGAAGGGCCTGAGTCCGAGCAGAAGAACCT | | NNN | | |
| | GCAGGACCTATCATTCANNNNNNNNAAAGTTCT | | | | |
| | CCCATCACATCAACCGGTGGCGCATTGCCACGA | | | | |
| | AGCAGGCCAATGGGGAGGACATCGATGTCACA | | | | |
| | CAACCAAATATAAGAATTGTTAGttattaatatattattatat | | | | |
| | aataattatt | | | | |
| Var0032 | aataatataatcccttagtctattagtttaGCCTTTATGCTGTGGTG | 19574 | ACCTAT | 19766 | ala2 |
| | TTGCGCCACGGTGGAGATTTGTCAAATACACAT | | CATTCA | | variant |
| | CATTAGGTTGCGACAAACGGCAGAAGCTGGAG | | NNNNN | | |
| | GAGGAAGGGCCTGAGTCCGAGCAGAAGAACCT | | NNN | | |
| | GCAGGACCTATCATTCANNNNNNNNAAAGTTCT | | | | |
| | CCCATCACATCAACCGGTGGCGCATTGCCACGA | | | | |
| | AGCAGGCCAATGGGGAGGACATCGATGTCACA | | | | |
| | CAACCAAATATAAGAATTGTTAGtaaactaatagactaa | | | | |
| | gggattatattatt | | | | |
| Var0033 | taatttacaggacagggactttactcgatcGCCTTTATGCTGTGGT | 19575 | ACCTAT | 19767 | ala2 |
| | GTTGCGCCACGGTGGAGATTTGTCAAATACACA | | CATTCA | | variant |
| | TCATTAGGTTGCGACAAACGGCAGAAGCTGGA | | NNNNN | | |
| | GGAGGAAGGGCCTGAGTCCGAGCAGAAGAACC | | NNN | | |
| | TGCAGGACCTATCATTCANNNNNNNNAAAGTTC | | | | |
| | TCCCATCACATCAACCGGTGGCGCATTGCCACG | | | | |
| | AAGCAGGCCAATGGGGAGGACATCGATGTCAC | | | | |
| | ACAACCAAATATAAGAATTGTTAGgatcgagtaaagtc | | | | |
| | cctgtcctgtaaatta | | | | |
| Var0034 | tatctggcaacggccagagagagcgcctgaGCCTTTATGCTGTG | 19576 | ACCTAT | 19768 | ala2 |
| | GTGTTGCGCCACGGTGGAGATTTGTCAAATACA | | CATTCA | | variant |
| | CATCATTAGGTTGCGACAAACGGCAGAAGCTG | | NNNNN | | |
| | GAGGAGGAAGGGCCTGAGTCCGAGCAGAAGAA | | NNN | | |
| | CCTGCAGGACCTATCATTCANNNNNNNNAAAG | | | | |
| | TTCTCCCATCACATCAACCGGTGGCGCATTGCC | | | | |
| | ACGAAGCAGGCCAATGGGGAGGACATCGATGT | | | | |
| | CACACAACCAAATATAAGAATTGTTAGtcaggcgct | | | | |
| | ctctctggccgttgccagata | | | | |
| Var0035 | gccgtcccgcgcggcctgcccaaacgccctGCCTTTATGCTGTG | 19577 | ACCTAT | 19769 | ala2 |
| | GTGTTGCGCCACGGTGGAGATTTGTCAAATACA | | CATTCA | | variant |
| | CATCATTAGGTTGCGACAAACGGCAGAAGCTG | | NNNNN | | |
| | GAGGAGGAAGGGCCTGAGTCCGAGCAGAAGAA | | NNN | | |
| | CCTGCAGGACCTATCATTCANNNNNNNNAAAG | | | | |
| | TTCTCCCATCACATCAACCGGTGGCGCATTGCC | | | | |
| | ACGAAGCAGGCCAATGGGGAGGACATCGATGT | | | | |
| | CACACAACCAAATATAAGAATTGTTAGagggcgttt | | | | |
| | gggcaggccgcgcgggacggc | | | | |
| Var0036 | cgccgccggccgggcgcgggcgccggcggcGCCTTTATGCTGT | 19578 | ACCTAT | 19770 | ala2 |
| | GGTGTTGCGCCACGGTGGAGATTTGTCAAATAC | | CATTCA | | variant |
| | ACATCATTAGGTTGCGACAAACGGCAGAAGCT | | NNNNN | | |
| | GGAGGAGGAAGGGCCTGAGTCCGAGCAGAAGA | | NNN | | |
| | ACCTGCAGGACCTATCATTCANNNNNNNNAAA | | | | |
| | GTTCTCCCATCACATCAACCGGTGGCGCATTGC | | | | |
| | CACGAAGCAGGCCAATGGGGAGGACATCGATG | | | | |
| | TCACACAACCAAATATAAGAATTGTTAGgccgccg | | | | |
| | gcgcccgcgcccggccggcggcg | | | | |
| Var0037 | aatatatataaatattaataataatattaataaatGCCTTTATGCTGTG | 19579 | ACCTAT | 19771 | ala2 |
| | GTGTTGCGCCACGGTGGAGATTTGTCAAATACA | | CATTCA | | variant |
| | CATCATTAGGTTGCGACAAACGGCAGAAGCTG | | NNNNN | | |
| | GAGGAGGAAGGGCCTGAGTCCGAGCAGAAGAA | | NNN | | |
| | CCTGCAGGACCTATCATTCANNNNNNNNAAAG | | | | |
| | TTCTCCCATCACATCAACCGGTGGCGCATTGCC | | | | |
| | ACGAAGCAGGCCAATGGGGAGGACATCGATGT | | | | |
| | CACACAACCAAATATAAGAATTGTTAGatttattaatat | | | | |
| | tattattaatatttatatatatt | | | | |
| Var0038 | ttaatatctaacaattattgaagttgctttatttcGCCTTTATGCTGTG | 19580 | ACCTAT | 19772 | ala2 |
| | GTGTTGCGCCACGGTGGAGATTTGTCAAATACA | | CATTCA | | variant |
| | CATCATTAGGTTGCGACAAACGGCAGAAGCTG | | NNNNN | | |
| | GAGGAGGAAGGGCCTGAGTCCGAGCAGAAGAA | | NNN | | |
| | CCTGCAGGACCTATCATTCANNNNNNNNAAAG | | | | |
| | TTCTCCCATCACATCAACCGGTGGCGCATTGCC | | | | |
| | ACGAAGCAGGCCAATGGGGAGGACATCGATGT | | | | |
| | CACACAACCAAATATAAGAATTGTTAGgaaataaag | | | | |
| | caacttcaataattgttagatattaa | | | | |
| Var0039 | cagttatagcgcgaactagtttgacctgatttgtaGCCTTTATGCTGT | 19581 | ACCTAT | 19773 | ala2 |
| | GGTGTTGCGCCACGGTGGAGATTTGTCAAATAC | | CATTCA | | variant |
| | ACATCATTAGGTTGCGACAAACGGCAGAAGCT | | NNNNN | | |
| | GGAGGAGGAAGGGCCTGAGTCCGAGCAGAAGA | | NNN | | |
| | ACCTGCAGGACCTATCATTCANNNNNNNNAAA | | | | |
| | GTTCTCCCATCACATCAACCGGTGGCGCATTGC | | | | |
| | CACGAAGCAGGCCAATGGGGAGGACATCGATG | | | | |
| | TCACACAACCAAATATAAGAATTGTTAGtacaaatc | | | | |
| | aggtcaaactagttcgcgctataactg | | | | |
| Var0040 | atagactggccgcccgtaggaacttgaggacgcacGCCTTTATGCT | 19582 | ACCTAT | 19774 | ala2 |
| | GTGGTGTTGCGCCACGGTGGAGATTTGTCAAAT | | CATTCA | | variant |
| | ACACATCATTAGGTTGCGACAAACGGCAGAAG | | NNNNN | | |
| | CTGGAGGAGGAAGGGCCTGAGTCCGAGCAGAA | | NNN | | |
| | GAACCTGCAGGACCTATCATTCANNNNNNNNA | | | | |
| | AAGTTCTCCCATCACATCAACCGGTGGCGCATT | | | | |
| | GCCACGAAGCAGGCCAATGGGGAGGACATCGA | | | | |
| | TGTCACACAACCAAATATAAGAATTGTTAGgtgcg | | | | |
| | tcctcaagttcctacgggggccagtctat | | | | |
| Var0041 | gcgagcgcgaggacgccacgagcacgcgccctgccGCCTTTATGC | 19583 | ACCTAT | 19775 | ala2 |
| | TGTGGTGTTGCGCCACGGTGGAGATTTGTCAAA | | CATTCA | | variant |
| | TACACATCATTAGGTTGCGACAAACGGCAGAA | | NNNNN | | |
| | GCTGGAGGAGGAAGGGCCTGAGTCCGAGCAGA | | NNN | | |
| | AGAACCTGCAGGACCTATCATTCANNNNNNNN | | | | |
| | AAAGTTCTCCCATCACATCAACCGGTGGCGCAT | | | | |
| | TGCCACGAAGCAGGCCAATGGGGAGGACATCG | | | | |
| | ATGTCACACAACCAAATATAAGAATTGTTAGggc | | | | |
| | agggcgcgtgctcgtggcgtcctcgcgctcgc | | | | |
| Var0042 | cggccggcgcccgggccggggggcccgcgggcggGCCTTTATG | 19584 | ACCTAT | 19776 | ala2 |
| | CTGTGGTGTTGCGCCACGGTGGAGATTTGTCAA | | CATTCA | | variant |
| | ATACACATCATTAGGTTGCGACAAACGGCAGA | | NNNNN | | |
| | AGCTGGAGGAGGAAGGGCCTGAGTCCGAGCAG | | NNN | | |
| | AAGAACCTGCAGGACCTATCATTCANNNNNNN | | | | |
| | NAAAGTTCTCCCATCACATCAACCGGTGGCGCA | | | | |
| | TTGCCACGAAGCAGGCCAATGGGGAGGACATC | | | | |
| | GATGTCACACAACCAAATATAAGAATTGTTAGc | | | | |
| | cgcccgcgggcccgcccggcccgggcgccggccg | | | | |
| Var0043 | catagagGCCTTTATGCTGTGGTGTTGCGCCACGGT | 19585 | ACCTAT | 19777 | ala2 |
| | GGAGATTTGTCAAATACACATCATTAGGTTGCG | | CATTCA | | variant |
| | ACAAACGGCAGAAGCTGGAGGAGGAAGGGCCT | | NNNNN | | |
| | GAGTCCGAGCAGAAGAACCTGCAGGACCTATC | | NNN | | |
| | ATTCANNNNNNNNAAAGTTCTCCCATCACATCA | | | | |
| | ACCGGTGGCGCATTGCCACGAAGCAGGCCAAT | | | | |
| | GGGGAGGACATCGATGTCACACAACCAAATAT | | | | |
| | AAGAATTGTTAGctctatg | | | | |
| Var0044 | CATAGATTTCTTGGCCTTTATGCTGTGGTGTTGC | 19586 | ACCTAT | 19778 | msD |
| | GCCACGGTGGAGATTTGTCAAATACACATCATT | | CATTCA | | stem |
| | AggttgcgcgcaattcgctacgctaccaatactgtgACAAACGGCA | | NNNNN | | variant |
| | GAAGCTGGAGGAGGAAGGGCCTGAGTCCGAGC | | NNN | | |
| | AGAAGAACCTGCAGGACCTATCATTCANNNNN | | | | |
| | NNNAAAGTTCTCCCATCACATCAACCGGTGGCG | | | | |
| | CATTGCCACGAAGCAGGCCAATGGGGAGGACA | | | | |
| | TCGATGTCAcacagtattggtgctacgctgaagtgtcacaaccAAA | | | | |
| | TATAAGAATTGTTAGCAAGAAATCTATG | | | | |
| Var0045 | CATAGATTTCTTGGCCTTTATGCTGTGGTGTTGC | 19587 | ACCTAT | 19779 | msD |
| | GCCACGGTGGAGATTTGTCAAATACACATCATT | | CATTCA | | stem |
| | AggttgcgcgcaattcgctacgctaccaataACAAACGGCAGAA | | NNNNN | | variant |
| | GCTGGAGGAGGAAGGGCCTGAGTCCGAGCAGA | | NNN | | |
| | AGAACCTGCAGGACCTATCATTCANNNNNNNN | | | | |
| | AAAGTTCTCCCATCACATCAACCGGTGGCGCAT | | | | |
| | TGCCACGAAGCAGGCCAATGGGGAGGACATCG | | | | |
| | ATGTCAtattggtgctacgctgaagtgtcacaaccAAATATAAG | | | | |
| | AATTGTTAGCAAGAAATCTATG | | | | |
| Var0046 | CATAGATTTCTTGGCCTTTATGCTGTGGTGTTGC | 19588 | ACCTAT | 19780 | msD |
| | GCCACGGTGGAGATTTGTCAAATACACATCATT | | CATTCA | | stem |
| | AggttgcgcgcaattcgctacgctacACAAACGGCAGAAGCT | | NNNNN | | variant |
| | GGAGGAGGAAGGGCCTGAGTCCGAGCAGAAGA | | NNN | | |
| | ACCTGCAGGACCTATCATTCANNNNNNNNAAA | | | | |
| | GTTCTCCCATCACATCAACCGGTGGCGCATTGC | | | | |
| | CACGAAGCAGGCCAATGGGGAGGACATCGATG | | | | |
| | TCAgtgctacgctgaagtgtcacaaccAAATATAAGAATTGT | | | | |
| | TAGCAAGAAATCTATG | | | | |
| Var0047 | CATAGATTTCTTGGCCTTTATGCTGTGGTGTTGC | 19589 | ACCTAT | 19781 | msD |
| | GCCACGGTGGAGATTTGTCAAATACACATCATT | | CATTCA | | stem |
| | AggttgcgcgcaattcgctacACAAACGGCAGAAGCTGGA | | NNNNN | | variant |
| | GGAGGAAGGGCCTGAGTCCGAGCAGAAGAACC | | NNN | | |
| | TGCAGGACCTATCATTCANNNNNNNNAAAGTTC | | | | |
| | TCCCATCACATCAACCGGTGGCGCATTGCCACG | | | | |
| | AAGCAGGCCAATGGGGAGGACATCGATGTCAta | | | | |
| | cgctgaagtgtcacaaccAAATATAAGAATTGTTAGCAA | | | | |
| | GAAATCTATG | | | | |
| Var0048 | CATAGATTTCTTGGCCTTTATGCTGTGGTGTTGC | 19590 | ACCTAT | 19782 | msD |
| | GCCACGGTGGAGATTTGTCAAATACACATCATT | | CATTCA | | stem |
| | AggttgcgcgcaattcACAAACGGCAGAAGCTGGAGGA | | NNNNN | | variant |
| | GGAAGGGCCTGAGTCCGAGCAGAAGAACCTGC | | NNN | | |
| | AGGACCTATCATTCANNNNNNNNAAAGTTCTCC | | | | |
| | CATCACATCAACCGGTGGCGCATTGCCACGAAG | | | | |
| | CAGGCCAATGGGGAGGACATCGATGTCAgaagtgt | | | | |
| | cacaaccAAATATAAGAATTGTTAGCAAGAAATCT | | | | |
| | ATG | | | | |
| Var0049 | CATAGATTTCTTGGCCTTTATGCTGTGGTGTTGC | 19591 | ACCTAT | 19783 | msD |
| | GCCACGGTGGAGATTTGTCAAATACACATCATT | | CATTCA | | stem |
| | AggttgcgcgcACAAACGGCAGAAGCTGGAGGAGG | | NNNNN | | variant |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCAgtgtcacaacc | | | | |
| | AAATATAAGAATTGTTAGCAAGAAATCTATG | | | | |
| Var0050 | CATAGATTTCTTGGCCTTTATGCTGTGGTGTTGC | 19592 | ACCTAT | 19784 | msD |
| | GCCACGGTGGAGATTTGTCAAATACACATCATT | | CATTCA | | stem |
| | AggttgACAAACGGCAGAAGCTGGAGGAGGAAGG | | NNNNN | | variant |
| | GCCTGAGTCCGAGCAGAAGAACCTGCAGGACC | | NNN | | |
| | TATCATTCANNNNNNNNAAAGTTCTCCCATCAC | | | | |
| | ATCAACCGGTGGCGCATTGCCACGAAGCAGGC | | | | |
| | CAATGGGGAGGACATCGATGTCAcaaccAAATAT | | | | |
| | AAGAATTGTTAGCAAGAAATCTATG | | | | |
| Var0051 | CATAGATTTCTTGGCCTTTATGCTGTGGTGTTGC | 19593 | ACCTAT | 19785 | msD |
| | GCCACGGTGGAGATTTGTCAAATACACATCATT | | CATTCA | | stem |
| | AtataaACAAACGGCAGAAGCTGGAGGAGGAAGG | | NNNNN | | variant |
| | GCCTGAGTCCGAGCAGAAGAACCTGCAGGACC | | NNN | | |
| | TATCATTCANNNNNNNNAAAGTTCTCCCATCAC | | | | |
| | ATCAACCGGTGGCGCATTGCCACGAAGCAGGC | | | | |
| | CAATGGGGAGGACATCGATGTCAttataAAATATA | | | | |
| | AGAATTGTTAGCAAGAAATCTATG | | | | |
| Var0052 | CATAGATTTCTTGGCCTTTATGCTGTGGTGTTGC | 19594 | ACCTAT | 19786 | msD |
| | GCCACGGTGGAGATTTGTCAAATACACATCATT | | CATTCA | | stem |
| | AtgataACAAACGGCAGAAGCTGGAGGAGGAAGG | | NNNNN | | variant |
| | GCCTGAGTCCGAGCAGAAGAACCTGCAGGACC | | NNN | | |
| | TATCATTCANNNNNNNNAAAGTTCTCCCATCAC | | | | |
| | ATCAACCGGTGGCGCATTGCCACGAAGCAGGC | | | | |
| | CAATGGGGAGGACATCGATGTCAtatcaAAATATA | | | | |
| | AGAATTGTTAGCAAGAAATCTATG | | | | |
| Var0053 | CATAGATTTCTTGGCCTTTATGCTGTGGTGTTGC | 19595 | ACCTAT | 19787 | msD |
| | GCCACGGTGGAGATTTGTCAAATACACATCATT | | CATTCA | | stem |
| | AtctacACAAACGGCAGAAGCTGGAGGAGGAAGG | | NNNNN | | variant |
| | GCCTGAGTCCGAGCAGAAGAACCTGCAGGACC | | NNN | | |
| | TATCATTCANNNNNNNNAAAGTTCTCCCATCAC | | | | |
| | ATCAACCGGTGGCGCATTGCCACGAAGCAGGC | | | | |
| | CAATGGGGAGGACATCGATGTCAgtagaAAATAT | | | | |
| | AAGAATTGTTAGCAAGAAATCTATG | | | | |
| Var0054 | CATAGATTTCTTGGCCTTTATGCTGTGGTGTTGC | 19596 | ACCTAT | 19788 | msD |
| | GCCACGGTGGAGATTTGTCAAATACACATCATT | | CATTCA | | stem |
| | AgatccACAAACGGCAGAAGCTGGAGGAGGAAGG | | NNNNN | | variant |
| | GCCTGAGTCCGAGCAGAAGAACCTGCAGGACC | | NNN | | |
| | TATCATTCANNNNNNNNAAAGTTCTCCCATCAC | | | | |
| | ATCAACCGGTGGCGCATTGCCACGAAGCAGGC | | | | |
| | CAATGGGGAGGACATCGATGTCAggatcAAATAT | | | | |
| | AAGAATTGTTAGCAAGAAATCTATG | | | | |
| Var0055 | CATAGATTTCTTGGCCTTTATGCTGTGGTGTTGC | 19597 | ACCTAT | 19789 | msD |
| | GCCACGGTGGAGATTTGTCAAATACACATCATT | | CATTCA | | stem |
| | AacgcgACAAACGGCAGAAGCTGGAGGAGGAAG | | NNNNN | | variant |
| | GGCCTGAGTCCGAGCAGAAGAACCTGCAGGAC | | NNN | | |
| | CTATCATTCANNNNNNNNAAAGTTCTCCCATCA | | | | |
| | CATCAACCGGTGGCGCATTGCCACGAAGCAGG | | | | |
| | CCAATGGGGAGGACATCGATGTCAcgcgtAAATA | | | | |
| | TAAGAATTGTTAGCAAGAAATCTATG | | | | |
| Var0056 | CATAGATTTCTTGGCCTTTATGCTGTGGTGTTGC | 19598 | ACCTAT | 19790 | msD |
| | GCCACGGTGGAGATTTGTCAAATACACATCATT | | CATTCA | | stem |
| | AcgcgcACAAACGGCAGAAGCTGGAGGAGGAAG | | NNNNN | | variant |
| | GGCCTGAGTCCGAGCAGAAGAACCTGCAGGAC | | NNN | | |
| | CTATCATTCANNNNNNNNAAAGTTCTCCCATCA | | | | |
| | CATCAACCGGTGGCGCATTGCCACGAAGCAGG | | | | |
| | CCAATGGGGAGGACATCGATGTCAgcgcgAAATA | | | | |
| | TAAGAATTGTTAGCAAGAAATCTATG | | | | |
| Var0057 | CATAGATTTCTTGGCCTTTATGCTGTGGTGTTGC | 19599 | ACCTAT | 19791 | msD |
| | GCCACGGTGGAGATTTGTCAAATACACATCATT | | CATTCA | | stem |
| | AtattaaatttACAAACGGCAGAAGCTGGAGGAGGAA | | NNNNN | | variant |
| | GGGCCTGAGTCCGAGCAGAAGAACCTGCAGGA | | NNN | | |
| | CCTATCATTCANNNNNNNNAAAGTTCTCCCATC | | | | |
| | ACATCAACCGGTGGCGCATTGCCACGAAGCAG | | | | |
| | GCCAATGGGGAGGACATCGATGTCAaaatttaataAA | | | | |
| | ATATAAGAATTGTTAGCAAGAAATCTATG | | | | |
| Var0058 | CATAGATTTCTTGGCCTTTATGCTGTGGTGTTGC | 19600 | ACCTAT | 19792 | msD |
| | GCCACGGTGGAGATTTGTCAAATACACATCATT | | CATTCA | | stem |
| | AtataatacgaACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCAtcgtattataA | | | | |
| | AATATAAGAATTGTTAGCAAGAAATCTATG | | | | |
| Var0059 | CATAGATTTCTTGGCCTTTATGCTGTGGTGTTGC | 19601 | ACCTAT | 19793 | msD |
| | GCCACGGTGGAGATTTGTCAAATACACATCATT | | CATTCA | | stem |
| | AttctgccaatACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCAattggcagaa | | | | |
| | AAATATAAGAATTGTTAGCAAGAAATCTATG | | | | |
| Var0060 | CATAGATTTCTTGGCCTTTATGCTGTGGTGTTGC | 19602 | ACCTAT | 19794 | msD |
| | GCCACGGTGGAGATTTGTCAAATACACATCATT | | CATTCA | | stem |
| | AtctcctcgagACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCActcgaggaga | | | | |
| | AAATATAAGAATTGTTAGCAAGAAATCTATG | | | | |
| Var0061 | CATAGATTTCTTGGCCTTTATGCTGTGGTGTTGC | 19603 | ACCTAT | 19795 | msD |
| | GCCACGGTGGAGATTTGTCAAATACACATCATT | | CATTCA | | stem |
| | AccgggttcgcACAAACGGCAGAAGCTGGAGGAGG | | NNNNN | | variant |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCAgcgaacccgg | | | | |
| | AAATATAAGAATTGTTAGCAAGAAATCTATG | | | | |
| Var0062 | CATAGATTTCTTGGCCTTTATGCTGTGGTGTTGC | 19604 | ACCTAT | 19796 | msD |
| | GCCACGGTGGAGATTTGTCAAATACACATCATT | | CATTCA | | stem |
| | AggccgggcccACAAACGGCAGAAGCTGGAGGAGG | | NNNNN | | variant |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCAgggcccggc | | | | |
| | cAAATATAAGAATTGTTAGCAAGAAATCTATG | | | | |
| Var0063 | CATAGATTTCTTGGCCTTTATGCTGTGGTGTTGC | 19605 | ACCTAT | 19797 | msL |
| | GCCACGGTGGAGATTTGTCAAATACACATCATT | | CATTCA | | stem |
| | AaaatttaattataaaACAAACGGCAGAAGCTGGAGGAG | | NNNNN | | variant |
| | GAAGGGCCTGAGTCCGAGCAGAAGAACCTGCA | | NNN | | |
| | GGACCTATCATTCANNNNNNNNAAAGTTCTCCC | | | | |
| | ATCACATCAACCGGTGGCGCATTGCCACGAAGC | | | | |
| | AGGCCAATGGGGAGGACATCGATGTCAtttataattaa | | | | |
| | atttAAATATAAGAATTGTTAGCAAGAAATCTATG | | | | |
| Var0064 | CATAGATTTCTTGGCCTTTATGCTGTGGTGTTGC | 19606 | ACCTAT | 19798 | msD |
| | GCCACGGTGGAGATTTGTCAAATACACATCATT | | CATTCA | | stem |
| | AatattctattacttgACAAACGGCAGAAGCTGGAGGAG | | NNNNN | | variant |
| | GAAGGGCCTGAGTCCGAGCAGAAGAACCTGCA | | NNN | | |
| | GGACCTATCATTCANNNNNNNNAAAGTTCTCCC | | | | |
| | ATCACATCAACCGGTGGCGCATTGCCACGAAGC | | | | |
| | AGGCCAATGGGGAGGACATCGATGTCAcaagtaata | | | | |
| | gaatatAAATATAAGAATTGTTAGCAAGAAATCTA | | | | |
| | TG | | | | |
| Var0065 | CATAGATTTCTTGGCCTTTATGCTGTGGTGTTGC | 19607 | ACCTAT | 19799 | msD |
| | GCCACGGTGGAGATTTGTCAAATACACATCATT | | CATTCA | | stem |
| | AgcgtatagtaatctgACAAACGGCAGAAGCTGGAGGA | | NNNNN | | variant |
| | GGAAGGGCCTGAGTCCGAGCAGAAGAACCTGC | | NNN | | |
| | AGGACCTATCATTCANNNNNNNNAAAGTTCTCC | | | | |
| | CATCACATCAACCGGTGGCGCATTGCCACGAAG | | | | |
| | CAGGCCAATGGGGAGGACATCGATGTCAcagattac | | | | |
| | tatacgcAAATATAAGAATTGTTAGCAAGAAATCT | | | | |
| | ATG | | | | |
| Var0066 | CATAGATTTCTTGGCCTTTATGCTGTGGTGTTGC | 19608 | ACCTAT | 19800 | msD |
| | GCCACGGTGGAGATTTGTCAAATACACATCATT | | CATTCA | | stem |
| | AaacgcgaaacgctggACAAACGGCAGAAGCTGGAGG | | NNNNN | | variant |
| | AGGAAGGGCCTGAGTCCGAGCAGAAGAACCTG | | NNN | | |
| | CAGGACCTATCATTCANNNNNNNNAAAGTTCTC | | | | |
| | CCATCACATCAACCGGTGGCGCATTGCCACGAA | | | | |
| | GCAGGCCAATGGGGAGGACATCGATGTCAccagc | | | | |
| | gtttcgcgttAAATATAAGAATTGTTAGCAAGAAATC | | | | |
| | TATG | | | | |
| Var0067 | CATAGATTTCTTGGCCTTTATGCTGTGGTGTTGC | 19609 | ACCTAT | 19801 | msD |
| | GCCACGGTGGAGATTTGTCAAATACACATCATT | | CATTCA | | stem |
| | AgaggcgggtgccgcaACAAACGGCAGAAGCTGGAGG | | NNNNN | | variant |
| | AGGAAGGGCCTGAGTCCGAGCAGAAGAACCTG | | NNN | | |
| | CAGGACCTATCATTCANNNNNNNNAAAGTTCTC | | | | |
| | CCATCACATCAACCGGTGGCGCATTGCCACGAA | | | | |
| | GCAGGCCAATGGGGAGGACATCGATGTCAtgcgg | | | | |
| | cacccgcctcAAATATAAGAATTGTTAGCAAGAAAT | | | | |
| | CTATG | | | | |
| Var0068 | CATAGATTTCTTGGCCTTTATGCTGTGGTGTTGC | 19610 | ACCTAT | 19802 | msD |
| | GCCACGGTGGAGATTTGTCAAATACACATCATT | | CATTCA | | stem |
| | AgcggccgcggggggACAAACGGCAGAAGCTGGAG | | NNNNN | | variant |
| | GAGGAAGGGCCTGAGTCCGAGCAGAAGAACCT | | NNN | | |
| | GCAGGACCTATCATTCANNNNNNNNAAAGTTCT | | | | |
| | CCCATCACATCAACCGGTGGCGCATTGCCACGA | | | | |
| | AGCAGGCCAATGGGGAGGACATCGATGTCAcccg | | | | |
| | cccgcggccgcAAATATAAGAATTGTTAGCAAGAAA | | | | |
| | TCTATG | | | | |
| Var0069 | CATAGATTTCTTGGCCTTTATGCTGTGGTGTTGC | 19611 | ACCTAT | 19803 | msD |
| | GCCACGGTGGAGATTTGTCAAATACACATCATT | | CATTCA | | stem |
| | AaatattatattaaatattatACAAACGGCAGAAGCTGGAG | | NNNNN | | variant |
| | GAGGAAGGGCCTGAGTCCGAGCAGAAGAACCT | | NNN | | |
| | GCAGGACCTATCATTCANNNNNNNNAAAGTTCT | | | | |
| | CCCATCACATCAACCGGTGGCGCATTGCCACGA | | | | |
| | AGCAGGCCAATGGGGAGGACATCGATGTCAataa | | | | |
| | tatttaatataatattAAATATAAGAATTGTTAGCAAGAA | | | | |
| | ATCTATG | | | | |
| Var0070 | CATAGATTTCTTGGCCTTTATGCTGTGGTGTTGC | 19612 | ACCTAT | 19804 | msD |
| | GCCACGGTGGAGATTTGTCAAATACACATCATT | | CATTCA | | stem |
| | AgaacaaactaaatataaatcACAAACGGCAGAAGCTGGA | | NNNNN | | variant |
| | GGAGGAAGGGCCTGAGTCCGAGCAGAAGAACC | | NNN | | |
| | TGCAGGACCTATCATTCANNNNNNNNAAAGTTC | | | | |
| | TCCCATCACATCAACCGGTGGCGCATTGCCACG | | | | |
| | AAGCAGGCCAATGGGGAGGACATCGATGTCAga | | | | |
| | tttatatttagtttgttcAAATATAAGAATTGTTAGCAAGAA | | | | |
| | ATCTATG | | | | |
| Var0071 | CATAGATTTCTTGGCCTTTATGCTGTGGTGTTGC | 19613 | ACCTAT | 19805 | msD |
| | GCCACGGTGGAGATTTGTCAAATACACATCATT | | CATTCA | | stem |
| | AcgatttctaagacttcggtaACAAACGGCAGAAGCTGGA | | NNNNN | | variant |
| | GGAGGAAGGGCCTGAGTCCGAGCAGAAGAACC | | NNN | | |
| | TGCAGGACCTATCATTCANNNNNNNNAAAGTTC | | | | |
| | TCCCATCACATCAACCGGTGGCGCATTGCCACG | | | | |
| | AAGCAGGCCAATGGGGAGGACATCGATGTCAta | | | | |
| | ccgaagtcttagaaatcgAAATATAAGAATTGTTAGCAAG | | | | |
| | AAATCTATG | | | | |
| Var0072 | CATAGATTTCTTGGCCTTTATGCTGTGGTGTTGC | 19614 | ACCTAT | 19806 | msD |
| | GCCACGGTGGAGATTTGTCAAATACACATCATT | | CATTCA | | stem |
| | AgtcagggaactccaggaggaACAAACGGCAGAAGCTGG | | NNNNN | | variant |
| | AGGAGGAAGGGCCTGAGTCCGAGCAGAAGAAC | | NNN | | |
| | CTGCAGGACCTATCATTCANNNNNNNNAAAGTT | | | | |
| | CTCCCATCACATCAACCGGTGGCGCATTGCCAC | | | | |
| | GAAGCAGGCCAATGGGGAGGACATCGATGTCAt | | | | |
| | cctcctggagttccctgacAAATATAAGAATTGTTAGCAA | | | | |
| | GAAATCTATG | | | | |
| Var0073 | CATAGATTTCTTGGCCTTTATGCTGTGGTGTTGC | 19615 | ACCTAT | 19807 | msD |
| | GCCACGGTGGAGATTTGTCAAATACACATCATT | | CATTCA | | stem |
| | AcgtgggccgcgcttgagccgACAAACGGCAGAAGCTGG | | NNNNN | | variant |
| | AGGAGGAAGGGCCTGAGTCCGAGCAGAAGAAC | | NNN | | |
| | CTGCAGGACCTATCATTCANNNNNNNNAAAGTT | | | | |
| | CTCCCATCACATCAACCGGTGGCGCATTGCCAC | | | | |
| | GAAGCAGGCCAATGGGGAGGACATCGATGTCA | | | | |
| | cggctcaagcgcggcccacgAAATATAAGAATTGTTAGC | | | | |
| | AAGAAATCTATG | | | | |
| Var0074 | CATAGATTTCTTGGCCTTTATGCTGTGGTGTTGC | 19616 | ACCTAT | 19808 | msD |
| | GCCACGGTGGAGATTTGTCAAATACACATCATT | | CATTCA | | stem |
| | AcggccgccggcgccggcgcgACAAACGGCAGAAGCTG | | NNNNN | | variant |
| | GAGGAGGAAGGGCCTGAGTCCGAGCAGAAGAA | | NNN | | |
| | CCTGCAGGACCTATCATTCANNNNNNNNAAAG | | | | |
| | TTCTCCCATCACATCAACCGGTGGCGCATTGCC | | | | |
| | ACGAAGCAGGCCAATGGGGAGGACATCGATGT | | | | |
| | CAcgcgccggcgccggcggccgAAATATAAGAATTGTTA | | | | |
| | GCAAGAAATCTATG | | | | |
| Var0075 | CATAGATTTCTTGGCCTTTATGCTGTGGTGTTGC | 19617 | ACCTAT | 19809 | msD |
| | GCCACGGTGGAGATTTGTCAAATACACATCATT | | CATTCA | | stem |
| | AattaatatataaatttaattatataACAAACGGCAGAAGCTGG | | NNNNN | | variant |
| | AGGAGGAAGGGCCTGAGTCCGAGCAGAAGAAC | | NNN | | |
| | CTGCAGGACCTATCATTCANNNNNNNNAAAGTT | | | | |
| | CTCCCATCACATCAACCGGTGGCGCATTGCCAC | | | | |
| | GAAGCAGGCCAATGGGGAGGACATCGATGTCAt | | | | |
| | atataattaaatttatatattaatAAATATAAGAATTGTTAGCA | | | | |
| | AGAAATCTATG | | | | |
| Var0076 | CATAGATTTCTTGGCCTTTATGCTGTGGTGTTGC | 19618 | ACCTAT | 19810 | msD |
| | GCCACGGTGGAGATTTGTCAAATACACATCATT | | CATTCA | | stem |
| | AgttatactaataagaattatctgaaACAAACGGCAGAAGCTG | | NNNNN | | variant |
| | GAGGAGGAAGGGCCTGAGTCCGAGCAGAAGAA | | NNN | | |
| | CCTGCAGGACCTATCATTCANNNNNNNNAAAG | | | | |
| | TTCTCCCATCACATCAACCGGTGGCGCATTGCC | | | | |
| | ACGAAGCAGGCCAATGGGGAGGACATCGATGT | | | | |
| | CAttcagataattcttattagtataacAAATATAAGAATTGTTA | | | | |
| | GCAAGAAATCTATG | | | | |
| Var0077 | CATAGATTTCTTGGCCTTTATGCTGTGGTGTTGC | 19619 | ACCTAT | 19811 | msD |
| | GCCACGGTGGAGATTTGTCAAATACACATCATT | | CATTCA | | stem |
| | AttaagatagaggcacttctagtgagACAAACGGCAGAAGCT | | NNNNN | | variant |
| | GGAGGAGGAAGGGCCTGAGTCCGAGCAGAAGA | | NNN | | |
| | ACCTGCAGGACCTATCATTCANNNNNNNNAAA | | | | |
| | GTTCTCCCATCACATCAACCGGTGGCGCATTGC | | | | |
| | CACGAAGCAGGCCAATGGGGAGGACATCGATG | | | | |
| | TCActcactagaagtgcctctatcttaaAAATATAAGAATTGT | | | | |
| | TAGCAAGAAATCTATG | | | | |
| Var0078 | CATAGATTTCTTGGCCTTTATGCTGTGGTGTTGC | 19620 | ACCTAT | 19812 | msD |
| | GCCACGGTGGAGATTTGTCAAATACACATCATT | | CATTCA | | stem |
| | AcaggcagcacgcacacagccgaaatACAAACGGCAGAAGC | | NNNNN | | variant |
| | TGGAGGAGGAAGGGCCTGAGTCCGAGCAGAAG | | NNN | | |
| | AACCTGCAGGACCTATCATTCANNNNNNNNAA | | | | |
| | AGTTCTCCCATCACATCAACCGGTGGCGCATTG | | | | |
| | CCACGAAGCAGGCCAATGGGGAGGACATCGAT | | | | |
| | GTCAatttcggctgtgtgcgtgctgcctgAAATATAAGAATTG | | | | |
| | TTAGCAAGAAATCTATG | | | | |
| Var0079 | CATAGATTTCTTGGCCTTTATGCTGTGGTGTTGC | 19621 | ACCTAT | 19813 | msD |
| | GCCACGGTGGAGATTTGTCAAATACACATCATT | | CATTCA | | stem |
| | AcaccactggcgccgccagcaggggACAAACGGCAGAAGC | | NNNNN | | variant |
| | TGGAGGAGGAAGGGCCTGAGTCCGAGCAGAAG | | NNN | | |
| | AACCTGCAGGACCTATCATTCANNNNNNNNAA | | | | |
| | AGTTCTCCCATCACATCAACCGGTGGCGCATTG | | | | |
| | CCACGAAGCAGGCCAATGGGGAGGACATCGAT | | | | |
| | GTCAccgcctgctggcggcgccagtggtgAAATATAAGAATT | | | | |
| | GTTAGCAAGAAATCTATG | | | | |
| Var0080 | CATAGATTTCTTGGCCTTTATGCTGTGGTGTTGC | 19622 | ACCTAT | 19814 | msD |
| | GCCACGGTGGAGATTTGTCAAATACACATCATT | | CATTCA | | stem |
| | AcgcggccgcgccgggcgcgcgcccgACAAACGGCAGAAG | | NNNNN | | variant |
| | CTGGAGGAGGAAGGGCCTGAGTCCGAGCAGAA | | NNN | | |
| | GAACCTGCAGGACCTATCATTCANNNNNNNNA | | | | |
| | AAGTTCTCCCATCACATCAACCGGTGGCGCATT | | | | |
| | GCCACGAAGCAGGCCAATGGGGAGGACATCGA | | | | |
| | TGTCAcgggcgcgcgcccggcgcggccgcgAAATATAAGA | | | | |
| | ATTGTTAGCAAGAAATCTATG | | | | |
| Var0081 | CATAGATTTCTTGGCCTTTATGCTGTGGTGTTGC | 19623 | ACCTAT | 19815 | msD |
| | GCCACGGTGGAGATTTGTCAAATACACATCATT | | CATTCA | | stem |
| | AaataattattatataataatatattaataaACAAACGGCAGAAGC | | NNNNN | | variant |
| | TGGAGGAGGAAGGGCCTGAGTCCGAGCAGAAG | | NNN | | |
| | AACCTGCAGGACCTATCATTCANNNNNNNNAA | | | | |
| | AGTTCTCCCATCACATCAACCGGTGGCGCATTG | | | | |
| | CCACGAAGCAGGCCAATGGGGAGGACATCGAT | | | | |
| | GTCAttattaatatattattatataataattattAAATATAAGAATT | | | | |
| | GTTAGCAAGAAATCTATG | | | | |
| Var0082 | CATAGATTTCTTGGCCTTTATGCTGTGGTGTTGC | 19624 | ACCTAT | 19816 | msD |
| | GCCACGGTGGAGATTTGTCAAATACACATCATT | | CATTCA | | stem |
| | AaataatataatcccttagtctattagtttaACAAACGGCAGAAGC | | NNNNN | | variant |
| | TGGAGGAGGAAGGGCCTGAGTCCGAGCAGAAG | | NNN | | |
| | AACCTGCAGGACCTATCATTCANNNNNNNNAA | | | | |
| | AGTTCTCCCATCACATCAACCGGTGGCGCATTG | | | | |
| | CCACGAAGCAGGCCAATGGGGAGGACATCGAT | | | | |
| | GTCAtaaactaatagactaagggattatattattAAATATAAGAAT | | | | |
| | TGTTAGCAAGAAATCTATG | | | | |
| Var0083 | CATAGATTTCTTGGCCTTTATGCTGTGGTGTTGC | 19625 | ACCTAT | 19817 | msD |
| | GCCACGGTGGAGATTTGTCAAATACACATCATT | | CATTCA | | stem |
| | AtaatttacaggacagggactttactcgatcACAAACGGCAGAA | | NNNNN | | variant |
| | GCTGGAGGAGGAAGGGCCTGAGTCCGAGCAGA | | NNN | | |
| | AGAACCTGCAGGACCTATCATTCANNNNNNNN | | | | |
| | AAAGTTCTCCCATCACATCAACCGGTGGCGCAT | | | | |
| | TGCCACGAAGCAGGCCAATGGGGAGGACATCG | | | | |
| | ATGTCAgatcgagtaaagtccctgtcctgtaaattaAAATATAAG | | | | |
| | AATTGTTAGCAAGAAATCTATG | | | | |
| Var0084 | CATAGATTTCTTGGCCTTTATGCTGTGGTGTTGC | 19626 | ACCTAT | 19818 | msD |
| | GCCACGGTGGAGATTTGTCAAATACACATCATT | | CATTCA | | stem |
| | AtatctggcaacggccagagagagcgcctgaACAAACGGCAGA | | NNNNN | | variant |
| | AGCTGGAGGAGGAAGGGCCTGAGTCCGAGCAG | | NNN | | |
| | AAGAACCTGCAGGACCTATCATTCANNNNNNN | | | | |
| | NAAAGTTCTCCCATCACATCAACCGGTGGCGCA | | | | |
| | TTGCCACGAAGCAGGCCAATGGGGAGGACATC | | | | |
| | GATGTCAtcaggcgctctctctggccgttgccagataAAATATAA | | | | |
| | GAATTGTTAGCAAGAAATCTATG | | | | |
| Var0085 | CATAGATTTCTTGGCCTTTATGCTGTGGTGTTGC | 19627 | ACCTAT | 19819 | msD |
| | GCCACGGTGGAGATTTGTCAAATACACATCATT | | CATTCA | | stem |
| | AgccgtcccggggcctgcccaaacgccctACAAACGGCAGA | | NNNNN | | variant |
| | AGCTGGAGGAGGAAGGGCCTGAGTCCGAGCAG | | NNN | | |
| | AAGAACCTGCAGGACCTATCATTCANNNNNNN | | | | |
| | NAAAGTTCTCCCATCACATCAACCGGTGGCGCA | | | | |
| | TTGCCACGAAGCAGGCCAATGGGGAGGACATC | | | | |
| | GATGTCAagggcgtttgggcaggccgcgcgggacggcAAATAT | | | | |
| | AAGAATTGTTAGCAAGAAATCTATG | | | | |
| Var0086 | CATAGATTTCTTGGCCTTTATGCTGTGGTGTTGC | 19628 | ACCTAT | 19820 | msD |
| | GCCACGGTGGAGATTTGTCAAATACACATCATT | | CATTCA | | stem |
| | AcgccgccggccgggcgcgggcgccggcggcACAAACGGCAG | | NNNNN | | variant |
| | AAGCTGGAGGAGGAAGGGCCTGAGTCCGAGCA | | NNN | | |
| | GAAGAACCTGCAGGACCTATCATTCANNNNNN | | | | |
| | NNAAAGTTCTCCCATCACATCAACCGGTGGCGC | | | | |
| | ATTGCCACGAAGCAGGCCAATGGGGAGGACAT | | | | |
| | CGATGTCAgccgccggcgcccgcgcccggccggcggcgAAAT | | | | |
| | ATAAGAATTGTTAGCAAGAAATCTATG | | | | |
| Var0087 | CATAGATTTCTTGGCCTTTATGCTGTGGTGTTGC | 19629 | ACCTAT | 19821 | msD |
| | GCCACGGTGGAGATTTGTCAAATACACATCATT | | CATTCA | | stem |
| | AaatatatataaatattaataataatattaataaatACAAACGGCAGA | | NNNNN | | variant |
| | AGCTGGAGGAGGAAGGGCCTGAGTCCGAGCAG | | NNN | | |
| | AAGAACCTGCAGGACCTATCATTCANNNNNNN | | | | |
| | NAAAGTTCTCCCATCACATCAACCGGTGGCGCA | | | | |
| | TTGCCACGAAGCAGGCCAATGGGGAGGACATC | | | | |
| | GATGTCAatttattaatattattattaatatttatatatattAAATATAA | | | | |
| | GAATTGTTAGCAAGAAATCTATG | | | | |
| Var0088 | CATAGATTTCTTGGCCTTTATGCTGTGGTGTTGC | 19630 | ACCTAT | 19822 | msD |
| | GCCACGGTGGAGATTTGTCAAATACACATCATT | | CATTCA | | stem |
| | AttaatatctaacaattattgaagttgctttatttcACAAACGGCAGA | | NNNNN | | variant |
| | AGCTGGAGGAGGAAGGGCCTGAGTCCGAGCAG | | NNN | | |
| | AAGAACCTGCAGGACCTATCATTCANNNNNNN | | | | |
| | NAAAGTTCTCCCATCACATCAACCGGTGGCGCA | | | | |
| | TTGCCACGAAGCAGGCCAATGGGGAGGACATC | | | | |
| | GATGTCAgaaataaagcaacttcaataattgttagatattaaAAATA | | | | |
| | TAAGAATTGTTAGCAAGAAATCTATG | | | | |
| Var0089 | CATAGATTTCTTGGCCTTTATGCTGTGGTGTTGC | 19631 | ACCTAT | 19823 | msD |
| | GCCACGGTGGAGATTTGTCAAATACACATCATT | | CATTCA | | stem |
| | AcagttatagcgcgaactagtttgacctgatttgtaACAAACGGCAG | | NNNNN | | variant |
| | AAGCTGGAGGAGGAAGGGCCTGAGTCCGAGCA | | NNN | | |
| | GAAGAACCTGCAGGACCTATCATTCANNNNNN | | | | |
| | NNAAAGTTCTCCCATCACATCAACCGGTGGCGC | | | | |
| | ATTGCCACGAAGCAGGCCAATGGGGAGGACAT | | | | |
| | CGATGTCAtacaaatcaggtcaaactagttcgcgctataactgAAAT | | | | |
| | ATAAGAATTGTTAGCAAGAAATCTATG | | | | |
| Var0090 | CATAGATTTCTTGGCCTTTATGCTGTGGTGTTGC | 19632 | ACCTAT | 19824 | msD |
| | GCCACGGTGGAGATTTGTCAAATACACATCATT | | CATTCA | | stem |
| | AatagactggccgcccgtaggaacttgaggacgcacACAAACGGC | | NNNNN | | variant |
| | AGAAGCTGGAGGAGGAAGGGCCTGAGTCCGAG | | NNN | | |
| | CAGAAGAACCTGCAGGACCTATCATTCANNNN | | | | |
| | NNNNAAAGTTCTCCCATCACATCAACCGGTGGC | | | | |
| | GCATTGCCACGAAGCAGGCCAATGGGGAGGAC | | | | |
| | ATCGATGTCAgtgcgtcctcaagttcctacgggggccagtctatA | | | | |
| | AATATAAGAATTGTTAGCAAGAAATCTATG | | | | |
| Var0091 | CATAGATTTCTTGGCCTTTATGCTGTGGTGTTGC | 19633 | ACCTAT | 19825 | msD |
| | GCCACGGTGGAGATTTGTCAAATACACATCATT | | CATTCA | | stem |
| | AgcgagcgcgaggacgccacgagcacgcgccctgccACAAACGG | | NNNNN | | variant |
| | CAGAAGCTGGAGGAGGAAGGGCCTGAGTCCGA | | NNN | | |
| | GCAGAAGAACCTGCAGGACCTATCATTCANNN | | | | |
| | NNNNNAAAGTTCTCCCATCACATCAACCGGTGG | | | | |
| | CGCATTGCCACGAAGCAGGCCAATGGGGAGGA | | | | |
| | CATCGATGTCAggcagggcgcgtgctcgtggcgtcctcgcgctcg | | | | |
| | cAAATATAAGAATTGTTAGCAAGAAATCTATG | | | | |
| Var0092 | CATAGATTTCTTGGCCTTTATGCTGTGGTGTTGC | 19634 | ACCTAT | 19826 | msD |
| | GCCACGGTGGAGATTTGTCAAATACACATCATT | | CATTCA | | stem |
| | AcggccggcgcccgggccggggggcccgcgggcggACAAACG | | NNNNN | | variant |
| | GCAGAAGCTGGAGGAGGAAGGGCCTGAGTCCG | | NNN | | |
| | AGCAGAAGAACCTGCAGGACCTATCATTCANN | | | | |
| | NNNNNNAAAGTTCTCCCATCACATCAACCGGTG | | | | |
| | GCGCATTGCCACGAAGCAGGCCAATGGGGAGG | | | | |
| | ACATCGATGTCAccgcccgcgggcccgcccggcccgggcgccg | | | | |
| | gccgAAATATAAGAATTGTTAGCAAGAAATCTAT | | | | |
| | G | | | | |
| Var0093 | CATAGATTTCTTGGCCTTTATGCTGTGGTGtgtCG | 19635 | ACCTAT | 19827 | msR |
| | CCACGGTGGAGATTTGTCAAATACACATCATTA | | CATTCA | | loop |
| | GGTTGCGACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0094 | CATAGATTTCTTGGCCTTTATGCTGTGGTGgttCG | 19636 | ACCTAT | 19828 | msR |
| | CCACGGTGGAGATTTGTCAAATACACATCATTA | | CATTCA | | loop |
| | GGTTGCGACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0095 | CATAGATTTCTTGGCCTTTATGCTGTGGTGttcCG | 19637 | ACCTAT | 19829 | msR |
| | CCACGGTGGAGATTTGTCAAATACACATCATTA | | CATTCA | | loop |
| | GGTTGCGACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0096 | CATAGATTTCTTGGCCTTTATGCTGTGGTGtctCG | 19638 | ACCTAT | 19830 | msR |
| | CCACGGTGGAGATTTGTCAAATACACATCATTA | | CATTCA | | loop |
| | GGTTGCGACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0097 | CATAGATTTCTTGGCCTTTATGCTGTGGTGcttCG | 19639 | ACCTAT | 19831 | msR |
| | CCACGGTGGAGATTTGTCAAATACACATCATTA | | CATTCA | | loop |
| | GGTTGCGACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0098 | CATAGATTTCTTGGCCTTTATGCTGTGGTGtttCG | 19640 | ACCTAT | 19832 | msR |
| | CCACGGTGGAGATTTGTCAAATACACATCATTA | | CATTCA | | loop |
| | GGTTGCGACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0099 | CATAGATTTCTTGGCCTTTATGCTGTGGTGttttCG | 19641 | ACCTAT | 19833 | msR |
| | CCACGGTGGAGATTTGTCAAATACACATCATTA | | CATTCA | | loop |
| | GGTTGCGACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0100 | CATAGATTTCTTGGCCTTTATGCTGTGGTGttgcC | 19642 | ACCTAT | 19834 | msR |
| | GCCACGGTGGAGATTTGTCAAATACACATCATT | | CATTCA | | loop |
| | AGGTTGCGACAAACGGCAGAAGCTGGAGGAGG | | NNNNN | | variant |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0101 | CATAGATTTCTTGGCCTTTATGCTGTGGTGcttgC | 19643 | ACCTAT | 19835 | msR |
| | GCCACGGTGGAGATTTGTCAAATACACATCATT | | CATTCA | | loop |
| | AGGTTGCGACAAACGGCAGAAGCTGGAGGAGG | | NNNNN | | variant |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0102 | CATAGATTTCTTGGCCTTTATtataaTTGttataGGAG | 19644 | ACCTAT | 19836 | msR |
| | ATTTGTCAAATACACATCATTAGGTTGCGACAA | | CATTCA | | stem |
| | ACGGCAGAAGCTGGAGGAGGAAGGGCCTGAGT | | NNNNN | | variant |
| | CCGAGCAGAAGAACCTGCAGGACCTATCATTCA | | NNN | | |
| | NNNNNNNNAAAGTTCTCCCATCACATCAACCG | | | | |
| | GTGGCGCATTGCCACGAAGCAGGCCAATGGGG | | | | |
| | AGGACATCGATGTCACACAACCAAATATAAGA | | | | |
| | ATTGTTAGCAAGAAATCTATG | | | | |
| Var0103 | CATAGATTTCTTGGCCTTTATtgataTTGtatcaGGAG | 19645 | ACCTAT | 19837 | msR |
| | ATTTGTCAAATACACATCATTAGGTTGCGACAA | | CATTCA | | stem |
| | ACGGCAGAAGCTGGAGGAGGAAGGGCCTGAGT | | NNNNN | | variant |
| | CCGAGCAGAAGAACCTGCAGGACCTATCATTCA | | NNN | | |
| | NNNNNNNNAAAGTTCTCCCATCACATCAACCG | | | | |
| | GTGGCGCATTGCCACGAAGCAGGCCAATGGGG | | | | |
| | AGGACATCGATGTCACACAACCAAATATAAGA | | | | |
| | ATTGTTAGCAAGAAATCTATG | | | | |
| Var0104 | CATAGATTTCTTGGCCTTTATtctacTTGgtagaGGAG | 19646 | ACCTAT | 19838 | msR |
| | ATTTGTCAAATACACATCATTAGGTTGCGACAA | | CATTCA | | stem |
| | ACGGCAGAAGCTGGAGGAGGAAGGGCCTGAGT | | NNNNN | | variant |
| | CCGAGCAGAAGAACCTGCAGGACCTATCATTCA | | NNN | | |
| | NNNNNNNNAAAGTTCTCCCATCACATCAACCG | | | | |
| | GTGGCGCATTGCCACGAAGCAGGCCAATGGGG | | | | |
| | AGGACATCGATGTCACACAACCAAATATAAGA | | | | |
| | ATTGTTAGCAAGAAATCTATG | | | | |
| Var0105 | CATAGATTTCTTGGCCTTTATgatccTTGggatcGGA | 19647 | ACCTAT | 19839 | msR |
| | GATTTGTCAAATACACATCATTAGGTTGCGACA | | CATTCA | | stem |
| | AACGGCAGAAGCTGGAGGAGGAAGGGCCTGAG | | NNNNN | | variant |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0106 | CATAGATTTCTTGGCCTTTATacgcgTTGcgcgtGGA | 19648 | ACCTAT | 19840 | msR |
| | GATTTGTCAAATACACATCATTAGGTTGCGACA | | CATTCA | | stem |
| | AACGGCAGAAGCTGGAGGAGGAAGGGCCTGAG | | NNNNN | | variant |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0107 | CATAGATTTCTTGGCCTTTATcgcgcTTGgcgcgGGA | 19649 | ACCTAT | 19841 | msR |
| | GATTTGTCAAATACACATCATTAGGTTGCGACA | | CATTCA | | stem |
| | AACGGCAGAAGCTGGAGGAGGAAGGGCCTGAG | | NNNNN | | variant |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0108 | CATAGATTTCTTGGCCTTTATtattaaatttTTGaaatttaat | 19650 | ACCTAT | 19842 | msR |
| | aGGAGATTTGTCAAATACACATCATTAGGTTGC | | CATTCA | | stem |
| | GACAAACGGCAGAAGCTGGAGGAGGAAGGGCC | | NNNNN | | variant |
| | TGAGTCCGAGCAGAAGAACCTGCAGGACCTAT | | NNN | | |
| | CATTCANNNNNNNNAAAGTTCTCCCATCACATC | | | | |
| | AACCGGTGGCGCATTGCCACGAAGCAGGCCAA | | | | |
| | TGGGGAGGACATCGATGTCACACAACCAAATA | | | | |
| | TAAGAATTGTTAGCAAGAAATCTATG | | | | |
| Var0109 | CATAGATTTCTTGGCCTTTATtataatacgaTTGtgtatta | 19651 | ACCTAT | 19843 | msR |
| | taGGAGATTTGTCAAATACACATCATTAGGTTGC | | CATTCA | | stem |
| | GACAAACGGCAGAAGCTGGAGGAGGAAGGGCC | | NNNNN | | variant |
| | TGAGTCCGAGCAGAAGAACCTGCAGGACCTAT | | NNN | | |
| | CATTCANNNNNNNNAAAGTTCTCCCATCACATC | | | | |
| | AACCGGTGGCGCATTGCCACGAAGCAGGCCAA | | | | |
| | TGGGGAGGACATCGATGTCACACAACCAAATA | | | | |
| | TAAGAATTGTTAGCAAGAAATCTATG | | | | |
| Var0110 | CATAGATTTCTTGGCCTTTATttctgccaatTTGattggca | 19652 | ACCTAT | 19844 | msR |
| | gaaGGAGATTTGTCAAATACACATCATTAGGTTG | | CATTCA | | stem |
| | CGACAAACGGCAGAAGCTGGAGGAGGAAGGGC | | NNNNN | | variant |
| | CTGAGTCCGAGCAGAAGAACCTGCAGGACCTA | | NNN | | |
| | TCATTCANNNNNNNNAAAGTTCTCCCATCACAT | | | | |
| | CAACCGGTGGCGCATTGCCACGAAGCAGGCCA | | | | |
| | ATGGGGAGGACATCGATGTCACACAACCAAAT | | | | |
| | ATAAGAATTGTTAGCAAGAAATCTATG | | | | |
| Var0111 | CATAGATTTCTTGGCCTTTATtctcctcgagTTGctcgag | 19653 | ACCTAT | 19845 | msR |
| | gagaGGAGATTTGTCAAATACACATCATTAGGTT | | CATTCA | | stem |
| | GCGACAAACGGCAGAAGCTGGAGGAGGAAGGG | | NNNNN | | variant |
| | CCTGAGTCCGAGCAGAAGAACCTGCAGGACCT | | NNN | | |
| | ATCATTCANNNNNNNNAAAGTTCTCCCATCACA | | | | |
| | TCAACCGGTGGCGCATTGCCACGAAGCAGGCC | | | | |
| | AATGGGGAGGACATCGATGTCACACAACCAAA | | | | |
| | TATAAGAATTGTTAGCAAGAAATCTATG | | | | |
| Var0112 | CATAGATTTCTTGGCCTTTATccgggttcgcTTGgcgaa | 19654 | ACCTAT | 19846 | msR |
| | cccggGGAGATTTGTCAAATACACATCATTAGGTT | | CATTCA | | stem |
| | GCGACAAACGGCAGAAGCTGGAGGAGGAAGGG | | NNNNN | | variant |
| | CCTGAGTCCGAGCAGAAGAACCTGCAGGACCT | | NNN | | |
| | ATCATTCANNNNNNNNAAAGTTCTCCCATCACA | | | | |
| | TCAACCGGTGGCGCATTGCCACGAAGCAGGCC | | | | |
| | AATGGGGAGGACATCGATGTCACACAACCAAA | | | | |
| | TATAAGAATTGTTAGCAAGAAATCTATG | | | | |
| Var0113 | CATAGATTTCTTGGCCTTTATggccgggcccTTGgggc | 19655 | ACCTAT | 19847 | msR |
| | ccggccGGAGATTTGTCAAATACACATCATTAGGT | | CATTCA | | stem |
| | TGCGACAAACGGCAGAAGCTGGAGGAGGAAGG | | NNNNN | | variant |
| | GCCTGAGTCCGAGCAGAAGAACCTGCAGGACC | | NNN | | |
| | TATCATTCANNNNNNNNAAAGTTCTCCCATCAC | | | | |
| | ATCAACCGGTGGCGCATTGCCACGAAGCAGGC | | | | |
| | CAATGGGGAGGACATCGATGTCACACAACCAA | | | | |
| | ATATAAGAATTGTTAGCAAGAAATCTATG | | | | |
| Var0114 | CATAGATTTCTTGGCCTTTATaaatttaattataaaTTGttta | 19656 | ACCTAT | 19848 | msR |
| | taattaaatttGGAGATTTGTCAAATACACATCATTAG | | CATTCA | | stem |
| | GTTGCGACAAACGGCAGAAGCTGGAGGAGGAA | | NNNNN | | variant |
| | GGGCCTGAGTCCGAGCAGAAGAACCTGCAGGA | | NNN | | |
| | CCTATCATTCANNNNNNNNAAAGTTCTCCCATC | | | | |
| | ACATCAACCGGTGGCGCATTGCCACGAAGCAG | | | | |
| | GCCAATGGGGAGGACATCGATGTCACACAACC | | | | |
| | AAATATAAGAATTGTTAGCAAGAAATCTATG | | | | |
| Var0115 | CATAGATTTCTTGGCCTTTATatattctattacttgTTGcaa | 19657 | ACCTAT | 19849 | msR |
| | gtaatagaatatGGAGATTTGTCAAATACACATCATTA | | CATTCA | | stem |
| | GGTTGCGACAAACGGCAGAAGCTGGAGGAGGA | | NNNNN | | variant |
| | AGGGCCTGAGTCCGAGCAGAAGAACCTGCAGG | | NNN | | |
| | ACCTATCATTCANNNNNNNNAAAGTTCTCCCAT | | | | |
| | CACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0116 | CATAGATTTCTTGGCCTTTATgcgtatagtaatctgTTGca | 19658 | ACCTAT | 19850 | msR |
| | gattactatacgcGGAGATTTGTCAAATACACATCATT | | CATTCA | | stem |
| | AGGTTGCGACAAACGGCAGAAGCTGGAGGAGG | | NNNNN | | variant |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CAAATATAAGAATTGTTAGCAAGAAATCTATG | | | | |
| Var0117 | CATAGATTTCTTGGCCTTTATaacgcgaaacgctggTTG | 19659 | ACCTAT | 19851 | msR |
| | ccagcgtttcgcgttGGAGATTTGTCAAATACACATCAT | | CATTCA | | stem |
| | TAGGTTGCGACAAACGGCAGAAGCTGGAGGAG | | NNNNN | | variant |
| | GAAGGGCCTGAGTCCGAGCAGAAGAACCTGCA | | NNN | | |
| | GGACCTATCATTCANNNNNNNNAAAGTTCTCCC | | | | |
| | ATCACATCAACCGGTGGCGCATTGCCACGAAGC | | | | |
| | AGGCCAATGGGGAGGACATCGATGTCACACAA | | | | |
| | CCAAATATAAGAATTGTTAGCAAGAAATCTATG | | | | |
| Var0118 | CATAGATTTCTTGGCCTTTATgaggcgggtgccgcaTTG | 19660 | ACCTAT | 19852 | msR |
| | tgcggcacccgcctcGGAGATTTGTCAAATACACATCA | | CATTCA | | stem |
| | TTAGGTTGCGACAAACGGCAGAAGCTGGAGGA | | NNNNN | | variant |
| | GGAAGGGCCTGAGTCCGAGCAGAAGAACCTGC | | NNN | | |
| | AGGACCTATCATTCANNNNNNNNAAAGTTCTCC | | | | |
| | CATCACATCAACCGGTGGCGCATTGCCACGAAG | | | | |
| | CAGGCCAATGGGGAGGACATCGATGTCACACA | | | | |
| | ACCAAATATAAGAATTGTTAGCAAGAAATCTAT | | | | |
| | G | | | | |
| Var0119 | CATAGATTTCTTGGCCTTTATgcggccgcgggcgggTT | 19661 | ACCTAT | 19853 | msR |
| | GcccgcccgcggccgcGGAGATTTGTCAAATACACATC | | CATTCA | | stem |
| | ATTAGGTTGCGACAAACGGCAGAAGCTGGAGG | | NNNNN | | variant |
| | AGGAAGGGCCTGAGTCCGAGCAGAAGAACCTG | | NNN | | |
| | CAGGACCTATCATTCANNNNNNNNAAAGTTCTC | | | | |
| | CCATCACATCAACCGGTGGCGCATTGCCACGAA | | | | |
| | GCAGGCCAATGGGGAGGACATCGATGTCACAC | | | | |
| | AACCAAATATAAGAATTGTTAGCAAGAAATCTA | | | | |
| | TG | | | | |
| Var0120 | CATAGATTTCTTGGCCTTTATaatattatattaaatattatTT | 19662 | ACCTAT | 19854 | msR |
| | GataatatttaatataatattGGAGATTTGTCAAATACACAT | | CATTCA | | stem |
| | CATTAGGTTGCGACAAACGGCAGAAGCTGGAG | | NNNNN | | variant |
| | GAGGAAGGGCCTGAGTCCGAGCAGAAGAACCT | | NNN | | |
| | GCAGGACCTATCATTCANNNNNNNNAAAGTTCT | | | | |
| | CCCATCACATCAACCGGTGGCGCATTGCCACGA | | | | |
| | AGCAGGCCAATGGGGAGGACATCGATGTCACA | | | | |
| | CAACCAAATATAAGAATTGTTAGCAAGAAATCT | | | | |
| | ATG | | | | |
| Var0121 | CATAGATTTCTTGGCCTTTATgaacaaactaaatataaatcT | 19663 | ACCTAT | 19855 | msR |
| | TGgatttatatttagtttgttcGGAGATTTGTCAAATACACAT | | CATTCA | | stem |
| | CATTAGGTTGCGACAAACGGCAGAAGCTGGAG | | NNNNN | | variant |
| | GAGGAAGGGCCTGAGTCCGAGCAGAAGAACCT | | NNN | | |
| | GCAGGACCTATCATTCANNNNNNNNAAAGTTCT | | | | |
| | CCCATCACATCAACCGGTGGCGCATTGCCACGA | | | | |
| | AGCAGGCCAATGGGGAGGACATCGATGTCACA | | | | |
| | CAACCAAATATAAGAATTGTTAGCAAGAAATCT | | | | |
| | ATG | | | | |
| Var0122 | CATAGATTTCTTGGCCTTTATcgatttctaagacttcggtaT | 19664 | ACCTAT | 19856 | msR |
| | TGtaccgaagtcttagaaatcgGGAGATTTGTCAAATACAC | | CATTCA | | stem |
| | ATCATTAGGTTGCGACAAACGGCAGAAGCTGG | | NNNNN | | variant |
| | AGGAGGAAGGGCCTGAGTCCGAGCAGAAGAAC | | NNN | | |
| | CTGCAGGACCTATCATTCANNNNNNNNAAAGTT | | | | |
| | CTCCCATCACATCAACCGGTGGCGCATTGCCAC | | | | |
| | GAAGCAGGCCAATGGGGAGGACATCGATGTCA | | | | |
| | CACAACCAAATATAAGAATTGTTAGCAAGAAA | | | | |
| | TCTATG | | | | |
| Var0123 | CATAGATTTCTTGGCCTTTATgtcagggaactccaggagg | 19665 | ACCTAT | 19857 | msR |
| | aTTGtcctcctggagttccctgacGGAGATTTGTCAAATAC | | CATTCA | | stem |
| | ACATCATTAGGTTGCGACAAACGGCAGAAGCT | | NNNNN | | variant |
| | GGAGGAGGAAGGGCCTGAGTCCGAGCAGAAGA | | NNN | | |
| | ACCTGCAGGACCTATCATTCANNNNNNNNAAA | | | | |
| | GTTCTCCCATCACATCAACCGGTGGCGCATTGC | | | | |
| | CACGAAGCAGGCCAATGGGGAGGACATCGATG | | | | |
| | TCACACAACCAAATATAAGAATTGTTAGCAAGA | | | | |
| | AATCTATG | | | | |
| Var0124 | CATAGATTTCTTGGCCTTTATcgtgggccgcgcttgagccg | 19666 | ACCTAT | 19858 | msR |
| | TTGcggctcaagcgcggcccacgGGAGATTTGTCAAATAC | | CATTCA | | stem |
| | ACATCATTAGGTTGCGACAAACGGCAGAAGCT | | NNNNN | | variant |
| | GGAGGAGGAAGGGCCTGAGTCCGAGCAGAAGA | | NNN | | |
| | ACCTGCAGGACCTATCATTCANNNNNNNNAAA | | | | |
| | GTTCTCCCATCACATCAACCGGTGGCGCATTGC | | | | |
| | CACGAAGCAGGCCAATGGGGAGGACATCGATG | | | | |
| | TCACACAACCAAATATAAGAATTGTTAGCAAGA | | | | |
| | AATCTATG | | | | |
| Var0125 | CATAGATTTCTTGGCCTTTATcggccgccggcgccggcg | 19667 | ACCTAT | 19859 | msR |
| | cgTTGcgcgccggcgccggcggccgGGAGATTTGTCAAAT | | CATTCA | | stem |
| | ACACATCATTAGGTTGCGACAAACGGCAGAAG | | NNNNN | | variant |
| | CTGGAGGAGGAAGGGCCTGAGTCCGAGCAGAA | | NNN | | |
| | GAACCTGCAGGACCTATCATTCANNNNNNNNA | | | | |
| | AAGTTCTCCCATCACATCAACCGGTGGCGCATT | | | | |
| | GCCACGAAGCAGGCCAATGGGGAGGACATCGA | | | | |
| | TGTCACACAACCAAATATAAGAATTGTTAGCAA | | | | |
| | GAAATCTATG | | | | |
| Var0126 | CATAGATTTCTTGGCCTTTATattaatatataaatttaattatat | 19668 | ACCTAT | 19860 | msR |
| | aTTGtatataattaaatttatatattaatGGAGATTTGTCAAATA | | CATTCA | | stem |
| | CACATCATTAGGTTGCGACAAACGGCAGAAGCT | | NNNNN | | variant |
| | GGAGGAGGAAGGGCCTGAGTCCGAGCAGAAGA | | NNN | | |
| | ACCTGCAGGACCTATCATTCANNNNNNNNAAA | | | | |
| | GTTCTCCCATCACATCAACCGGTGGCGCATTGC | | | | |
| | CACGAAGCAGGCCAATGGGGAGGACATCGATG | | | | |
| | TCACACAACCAAATATAAGAATTGTTAGCAAGA | | | | |
| | AATCTATG | | | | |
| Var0127 | CATAGATTTCTTGGCCTTTATgttatactaataagaattatctg | 19669 | ACCTAT | 19861 | msR |
| | aaTTGttcagataattcttattagtataacGGAGATTTGTCAAAT | | CATTCA | | stem |
| | ACACATCATTAGGTTGCGACAAACGGCAGAAG | | NNNNN | | variant |
| | CTGGAGGAGGAAGGGCCTGAGTCCGAGCAGAA | | NNN | | |
| | GAACCTGCAGGACCTATCATTCANNNNNNNNA | | | | |
| | AAGTTCTCCCATCACATCAACCGGTGGCGCATT | | | | |
| | GCCACGAAGCAGGCCAATGGGGAGGACATCGA | | | | |
| | TGTCACACAACCAAATATAAGAATTGTTAGCAA | | | | |
| | GAAATCTATG | | | | |
| Var0128 | CATAGATTTCTTGGCCTTTATttaagatagaggcacttctagt | 19670 | ACCTAT | 19862 | msR |
| | gagTTGctcactagaagtgcctctatcttaaGGAGATTTGTCAA | | CATTCA | | stem |
| | ATACACATCATTAGGTTGCGACAAACGGCAGA | | NNNNN | | variant |
| | AGCTGGAGGAGGAAGGGCCTGAGTCCGAGCAG | | NNN | | |
| | AAGAACCTGCAGGACCTATCATTCANNNNNNN | | | | |
| | NAAAGTTCTCCCATCACATCAACCGGTGGCGCA | | | | |
| | TTGCCACGAAGCAGGCCAATGGGGAGGACATC | | | | |
| | GATGTCACACAACCAAATATAAGAATTGTTAGC | | | | |
| | AAGAAATCTATG | | | | |
| Var0129 | CATAGATTTCTTGGCCTTTATcaggcagcacgcacacagc | 19671 | ACCTAT | 19863 | msR |
| | cgaaatTTGatttcggctgtgtgcgtgctgcctgGGAGATTTGTC | | CATTCA | | stem |
| | AAATACACATCATTAGGTTGCGACAAACGGCA | | NNNNN | | variant |
| | GAAGCTGGAGGAGGAAGGGCCTGAGTCCGAGC | | NNN | | |
| | AGAAGAACCTGCAGGACCTATCATTCANNNNN | | | | |
| | NNNAAAGTTCTCCCATCACATCAACCGGTGGCG | | | | |
| | CATTGCCACGAAGCAGGCCAATGGGGAGGACA | | | | |
| | TCGATGTCACACAACCAAATATAAGAATTGTTA | | | | |
| | GCAAGAAATCTATG | | | | |
| Var0130 | CATAGATTTCTTGGCCTTTATcaccactggcgccgccagc | 19672 | ACCTAT | 19864 | msR |
| | aggcggTTGccgcctgctggcggcgccagtggtgGGAGATTTGT | | CATTCA | | stem |
| | CAAATACACATCATTAGGTTGCGACAAACGGCA | | NNNNN | | variant |
| | GAAGCTGGAGGAGGAAGGGCCTGAGTCCGAGC | | NNN | | |
| | AGAAGAACCTGCAGGACCTATCATTCANNNNN | | | | |
| | NNNAAAGTTCTCCCATCACATCAACCGGTGGCG | | | | |
| | CATTGCCACGAAGCAGGCCAATGGGGAGGACA | | | | |
| | TCGATGTCACACAACCAAATATAAGAATTGTTA | | | | |
| | GCAAGAAATCTATG | | | | |
| Var0131 | CATAGATTTCTTGGCCTTTATcgcggccgcgccgggcgc | 19673 | ACCTAT | 19865 | msR |
| | gcgcccgTTGcgggcgcgcgcccggcgcggccgcgGGAGATTT | | CATTCA | | stem |
| | GTCAAATACACATCATTAGGTTGCGACAAACGG | | NNNNN | | variant |
| | CAGAAGCTGGAGGAGGAAGGGCCTGAGTCCGA | | NNN | | |
| | GCAGAAGAACCTGCAGGACCTATCATTCANNN | | | | |
| | NNNNNAAAGTTCTCCCATCACATCAACCGGTGG | | | | |
| | CGCATTGCCACGAAGCAGGCCAATGGGGAGGA | | | | |
| | CATCGATGTCACACAACCAAATATAAGAATTGT | | | | |
| | TAGCAAGAAATCTATG | | | | |
| Var0132 | CATAGATTTCTTGGCCTTTATaataattattatataataatatat | 19674 | ACCTAT | 19866 | msR |
| | taataaTTGttattaatatattattatataataattattGGAGATTTGTC | | CATTCA | | stem |
| | AAATACACATCATTAGGTTGCGACAAACGGCA | | NNNNN | | variant |
| | GAAGCTGGAGGAGGAAGGGCCTGAGTCCGAGC | | NNN | | |
| | AGAAGAACCTGCAGGACCTATCATTCANNNNN | | | | |
| | NNNAAAGTTCTCCCATCACATCAACCGGTGGCG | | | | |
| | CATTGCCACGAAGCAGGCCAATGGGGAGGACA | | | | |
| | TCGATGTCACACAACCAAATATAAGAATTGTTA | | | | |
| | GCAAGAAATCTATG | | | | |
| Var0133 | CATAGATTTCTTGGCCTTTATaataatataatcccttagtctat | 19675 | ACCTAT | 19867 | msR |
| | tagtttaTTGtaaactaatagactaagggattatattattGGAGATTTG | | CATTCA | | stem |
| | TCAAATACACATCATTAGGTTGCGACAAACGGC | | NNNNN | | variant |
| | AGAAGCTGGAGGAGGAAGGGCCTGAGTCCGAG | | NNN | | |
| | CAGAAGAACCTGCAGGACCTATCATTCANNNN | | | | |
| | NNNNAAAGTTCTCCCATCACATCAACCGGTGGC | | | | |
| | GCATTGCCACGAAGCAGGCCAATGGGGAGGAC | | | | |
| | ATCGATGTCACACAACCAAATATAAGAATTGTT | | | | |
| | AGCAAGAAATCTATG | | | | |
| Var0134 | CATAGATTTCTTGGCCTTTATtaatttacaggacagggacttt | 19676 | ACCTAT | 19868 | msR |
| | actcgatcTTGgatcgagtaaagtccctgtcctgtaaattaGGAGATT | | CATTCA | | stem |
| | TGTCAAATACACATCATTAGGTTGCGACAAACG | | NNNNN | | variant |
| | GCAGAAGCTGGAGGAGGAAGGGCCTGAGTCCG | | NNN | | |
| | AGCAGAAGAACCTGCAGGACCTATCATTCANN | | | | |
| | NNNNNNAAAGTTCTCCCATCACATCAACCGGTG | | | | |
| | GCGCATTGCCACGAAGCAGGCCAATGGGGAGG | | | | |
| | ACATCGATGTCACACAACCAAATATAAGAATTG | | | | |
| | TTAGCAAGAAATCTATG | | | | |
| Var0135 | CATAGATTTCTTGGCCTTTATtatctggcaacggccagaga | 19677 | ACCTAT | 19869 | msR |
| | gagcgcctgaTTGtcaggcgctctctctggccgttgccagataGGAGA | | CATTCA | | stem |
| | TTTGTCAAATACACATCATTAGGTTGCGACAAA | | NNNNN | | variant |
| | CGGCAGAAGCTGGAGGAGGAAGGGCCTGAGTC | | NNN | | |
| | CGAGCAGAAGAACCTGCAGGACCTATCATTCA | | | | |
| | NNNNNNNNAAAGTTCTCCCATCACATCAACCG | | | | |
| | GTGGCGCATTGCCACGAAGCAGGCCAATGGGG | | | | |
| | AGGACATCGATGTCACACAACCAAATATAAGA | | | | |
| | ATTGTTAGCAAGAAATCTATG | | | | |
| Var0136 | CATAGATTTCTTGGCCTTTATgccgtcccgcgcggcctgcc | 19678 | ACCTAT | 19870 | msR |
| | caaacgccctTTGagggcgtttgggcaggccgcgcgggacggcGGA | | CATTCA | | stem |
| | GATTTGTCAAATACACATCATTAGGTTGCGACA | | NNNNN | | variant |
| | AACGGCAGAAGCTGGAGGAGGAAGGGCCTGAG | | NNN | | |
| | TCCGAGCAGAAGAACCTGCAGGACCTATCATTC | | | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0137 | CATAGATTTCTTGGCCTTTATcgccgccggccgggcgcg | 19679 | ACCTAT | 19871 | msR |
| | ggcgccggcggcTTGgccgccggcgcccgcgcccggccggcggcgG | | CATTCA | | stem |
| | GAGATTTGTCAAATACACATCATTAGGTTGCGA | | NNNNN | | variant |
| | CAAACGGCAGAAGCTGGAGGAGGAAGGGCCTG | | NNN | | |
| | AGTCCGAGCAGAAGAACCTGCAGGACCTATCA | | | | |
| | TTCANNNNNNNNAAAGTTCTCCCATCACATCAA | | | | |
| | CCGGTGGCGCATTGCCACGAAGCAGGCCAATG | | | | |
| | GGGAGGACATCGATGTCACACAACCAAATATA | | | | |
| | AGAATTGTTAGCAAGAAATCTATG | | | | |
| Var0138 | CATAGATTTCTTGGCCTTTATaatatatataaatattaataata | 19680 | ACCTAT | 19872 | msR |
| | atattaataaatTTGatttattaatattattattaatatttatatatattGGAGAT | | CATTCA | | stem |
| | TTGTCAAATACACATCATTAGGTTGCGACAAAC | | NNNNN | | variant |
| | GGCAGAAGCTGGAGGAGGAAGGGCCTGAGTCC | | NNN | | |
| | GAGCAGAAGAACCTGCAGGACCTATCATTCAN | | | | |
| | NNNNNNNAAAGTTCTCCCATCACATCAACCGGT | | | | |
| | GGCGCATTGCCACGAAGCAGGCCAATGGGGAG | | | | |
| | GACATCGATGTCACACAACCAAATATAAGAATT | | | | |
| | GTTAGCAAGAAATCTATG | | | | |
| Var0139 | CATAGATTTCTTGGCCTTTATttaatatctaacaattattgaag | 19681 | ACCTAT | 19873 | msR |
| | ttgctttatttcTTGgaaataaagcaacttcaataattgttagatattaaGGA | | CATTCA | | stem |
| | GATTTGTCAAATACACATCATTAGGTTGCGACA | | NNNNN | | variant |
| | AACGGCAGAAGCTGGAGGAGGAAGGGCCTGAG | | NNN | | |
| | TCCGAGCAGAAGAACCTGCAGGACCTATCATTC | | | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0140 | CATAGATTTCTTGGCCTTTATcagttatagcgcgaactagttt | 19682 | ACCTAT | 19874 | msR |
| | gacctgatttgtaTTGtacaaatcaggtcaaactagttcgcgctataactgG | | CATTCA | | stem |
| | GAGATTTGTCAAATACACATCATTAGGTTGCGA | | NNNNN | | variant |
| | CAAACGGCAGAAGCTGGAGGAGGAAGGGCCTG | | NNN | | |
| | AGTCCGAGCAGAAGAACCTGCAGGACCTATCA | | | | |
| | TTCANNNNNNNNAAAGTTCTCCCATCACATCAA | | | | |
| | CCGGTGGCGCATTGCCACGAAGCAGGCCAATG | | | | |
| | GGGAGGACATCGATGTCACACAACCAAATATA | | | | |
| | AGAATTGTTAGCAAGAAATCTATG | | | | |
| Var0141 | CATAGATTTCTTGGCCTTTATatagactggccgcccgtagg | 19683 | ACCTAT | 19875 | msR |
| | aacttgaggacgcacTTGgtgcgtcctcaagttcctacgggcggccagtct | | CATTCA | | stem |
| | atGGAGATTTGTCAAATACACATCATTAGGTTGC | | NNNNN | | variant |
| | GACAAACGGCAGAAGCTGGAGGAGGAAGGGCC | | NNN | | |
| | TGAGTCCGAGCAGAAGAACCTGCAGGACCTAT | | | | |
| | CATTCANNNNNNNNAAAGTTCTCCCATCACATC | | | | |
| | AACCGGTGGCGCATTGCCACGAAGCAGGCCAA | | | | |
| | TGGGGAGGACATCGATGTCACACAACCAAATA | | | | |
| | TAAGAATTGTTAGCAAGAAATCTATG | | | | |
| Var0142 | CATAGATTTCTTGGCCTTTATgcgagcgcgaggacgccac | 19684 | ACCTAT | 19876 | msR |
| | gagcacgcgccctgccTTGggcagggcgcgtgctcgtggcgtcctcgcg | | CATTCA | | stem |
| | ctcgcGGAGATTTGTCAAATACACATCATTAGGTT | | NNNNN | | variant |
| | GCGACAAACGGCAGAAGCTGGAGGAGGAAGGG | | NNN | | |
| | CCTGAGTCCGAGCAGAAGAACCTGCAGGACCT | | | | |
| | ATCATTCANNNNNNNNAAAGTTCTCCCATCACA | | | | |
| | TCAACCGGTGGCGCATTGCCACGAAGCAGGCC | | | | |
| | AATGGGGAGGACATCGATGTCACACAACCAAA | | | | |
| | TATAAGAATTGTTAGCAAGAAATCTATG | | | | |
| Var0143 | CATAGATTTCTTGGCCTTTATcggccggcgcccgggccg | 19685 | ACCTAT | 19877 | msR |
| | ggcgggcccgcgggcggTTGccgcccgcgggcccgcccggcccgggc | | CATTCA | | stem |
| | gccggccgGGAGATTTGTCAAATACACATCATTAG | | NNNNN | | variant |
| | GTTGCGACAAACGGCAGAAGCTGGAGGAGGAA | | NNN | | |
| | GGGCCTGAGTCCGAGCAGAAGAACCTGCAGGA | | | | |
| | CCTATCATTCANNNNNNNNAAAGTTCTCCCATC | | | | |
| | ACATCAACCGGTGGCGCATTGCCACGAAGCAG | | | | |
| | GCCAATGGGGAGGACATCGATGTCACACAACC | | | | |
| | AAATATAAGAATTGTTAGCAAGAAATCTATG | | | | |
| Var0144 | CATAGATTTCTTGgcatGCTGTGGTGTTGCGCCAC | 19686 | ACCTAT | 19878 | Minimal |
| | GGTGGAGATTTGTCAAATACACATCATTAGGTT | | CATTCA | | variant |
| | GCGACAAACGGCAGAAGCTGGAGGAGGAAGGG | | NNNNN | | |
| | CCTGAGTCCGAGCAGAAGAACCTGCAGGACCT | | NNN | | |
| | ATCATTCANNNNNNNNAAAGTTCTCCCATCACA | | | | |
| | TCAACCGGTGGCGCATTGCCACGAAGCAGGCC | | | | |
| | AATGGGGAGGACATCGATGTCACACAACCAAA | | | | |
| | TATAAGAATTGTTAGCAAGAAATCTATG | | | | |
| Var0145 | CATAGATTTCTTGGCCTTTATGCTGTGGTGTTGC | 19687 | ACCTAT | 19879 | Minimal |
| | GCCACGGTggtgtcaaatacacatcattaGGTTGCGACAAA | | CATTCA | | variant |
| | CGGCAGAAGCTGGAGGAGGAAGGGCCTGAGTC | | NNNNN | | |
| | CGAGCAGAAGAACCTGCAGGACCTATCATTCA | | NNN | | |
| | NNNNNNNNAAAGTTCTCCCATCACATCAACCG | | | | |
| | GTGGCGCATTGCCACGAAGCAGGCCAATGGGG | | | | |
| | AGGACATCGATGTCACACAACCAAATATAAGA | | | | |
| | ATTGTTAGCAAGAAATCTATG | | | | |
| Var0146 | CATAGATTTCTTGGCCTTTATGCTGTGGTGTTGC | 19688 | ACCTAT | 19880 | Minimal |
| | GCCACGGTggagatttgtcaaatacactaGGTTGCGACAAA | | CATTCA | | variant |
| | CGGCAGAAGCTGGAGGAGGAAGGGCCTGAGTC | | NNNNN | | |
| | CGAGCAGAAGAACCTGCAGGACCTATCATTCA | | NNN | | |
| | NNNNNNNNAAAGTTCTCCCATCACATCAACCG | | | | |
| | GTGGCGCATTGCCACGAAGCAGGCCAATGGGG | | | | |
| | AGGACATCGATGTCACACAACCAAATATAAGA | | | | |
| | ATTGTTAGCAAGAAATCTATG | | | | |
| Var0147 | CATAGATTTCTTGGCCTTTATGCTGTGGTGTTGC | 19689 | ACCTAT | 19881 | Minimal |
| | GCCACGGTggaatacacatcattaGGTTGCGACAAACGG | | CATTCA | | variant |
| | CAGAAGCTGGAGGAGGAAGGGCCTGAGTCCGA | | NNNNN | | |
| | GCAGAAGAACCTGCAGGACCTATCATTCANNN | | NNN | | |
| | NNNNNAAAGTTCTCCCATCACATCAACCGGTGG | | | | |
| | CGCATTGCCACGAAGCAGGCCAATGGGGAGGA | | | | |
| | CATCGATGTCACACAACCAAATATAAGAATTGT | | | | |
| | TAGCAAGAAATCTATG | | | | |
| Var0148 | CATAGATTTCTTGGCCTTTATGCTGTGGTGTTGC | 19690 | ACCTAT | 19882 | Minimal |
| | GCCACGGTggagatttgtcaaataGGTTGCGACAAACGG | | CATTCA | | variant |
| | CAGAAGCTGGAGGAGGAAGGGCCTGAGTCCGA | | NNNNN | | |
| | GCAGAAGAACCTGCAGGACCTATCATTCANNN | | NNN | | |
| | NNNNNAAAGTTCTCCCATCACATCAACCGGTGG | | | | |
| | CGCATTGCCACGAAGCAGGCCAATGGGGAGGA | | | | |
| | CATCGATGTCACACAACCAAATATAAGAATTGT | | | | |
| | TAGCAAGAAATCTATG | | | | |
| Var0149 | CATAGATTTCTTGGCCTTTATGCTGTGGTGTTGC | 19691 | ACCTAT | 19883 | Minimal |
| | GCCACGGTggcatcattaGGTTGCGACAAACGGCAG | | CATTCA | | variant |
| | AAGCTGGAGGAGGAAGGGCCTGAGTCCGAGCA | | NNNNN | | |
| | GAAGAACCTGCAGGACCTATCATTCANNNNNN | | NNN | | |
| | NNAAAGTTCTCCCATCACATCAACCGGTGGCGC | | | | |
| | ATTGCCACGAAGCAGGCCAATGGGGAGGACAT | | | | |
| | CGATGTCACACAACCAAATATAAGAATTGTTAG | | | | |
| | CAAGAAATCTATG | | | | |
| Var0150 | CATAGATTTCTTGGCCTTTATGCTGTGGTGTTGC | 19692 | ACCTAT | 19884 | Minimal |
| | GCCACGGTggagattttaGGTTGCGACAAACGGCAGA | | CATTCA | | variant |
| | AGCTGGAGGAGGAAGGGCCTGAGTCCGAGCAG | | NNNNN | | |
| | AAGAACCTGCAGGACCTATCATTCANNNNNNN | | NNN | | |
| | NAAAGTTCTCCCATCACATCAACCGGTGGCGCA | | | | |
| | TTGCCACGAAGCAGGCCAATGGGGAGGACATC | | | | |
| | GATGTCACACAACCAAATATAAGAATTGTTAGC | | | | |
| | AAGAAATCTATG | | | | |
| Var0151 | CATAGATTTCTTGGCCTTTATGCTGTGGTGTTGC | 19693 | ACCTAT | 19885 | Minimal |
| | GCCACGGTggtaGGTTGCGACAAACGGCAGAAGC | | CATTCA | | variant |
| | TGGAGGAGGAAGGGCCTGAGTCCGAGCAGAAG | | NNNNN | | |
| | AACCTGCAGGACCTATCATTCANNNNNNNNAA | | NNN | | |
| | AGTTCTCCCATCACATCAACCGGTGGCGCATTG | | | | |
| | CCACGAAGCAGGCCAATGGGGAGGACATCGAT | | | | |
| | GTCACACAACCAAATATAAGAATTGTTAGCAAG | | | | |
| | AAATCTATG | | | | |
| Var0152 | CATAGATTTCTTGGCCTTTATGCTGTGGTGTTGC | 19694 | ACCTAT | 19886 | Minimal |
| | GCCACGGTGGAGATTTGTCAAATACACATCATT | | CATTCA | | variant |
| | AGGTTGCGACAAACGGCAGAAGCTGGAGGAGG | | NNNNN | | |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CaaagaattgttagCAAGAAATCTATG | | | | |
| Var0153 | CATAGATTTCTTGGCCTTTATGCTGTGGTGTTGC | 19695 | ACCTAT | 19887 | Minimal |
| | GCCACGGTGGAGATTTGTCAAATACACATCATT | | CATTCA | | variant |
| | AGGTTGCGACAAACGGCAGAAGCTGGAGGAGG | | | | |
| | AAGGGCCTGAGTCCGAGCAGAAGAACCTGCAG | | NNNNN | | |
| | GACCTATCATTCANNNNNNNNAAAGTTCTCCCA | | NNN | | |
| | TCACATCAACCGGTGGCGCATTGCCACGAAGCA | | | | |
| | GGCCAATGGGGAGGACATCGATGTCACACAAC | | | | |
| | CaaatataagaaagCAAGAAATCTATG | | | | |
| Var0154 | CATAGATTTCTTGGCCTTTATGCTGTGGTGTTGC | 19696 | ACCTAT | 19888 | Minimal |
| | GCCACGGTGGAGATTTGTCAAATACACATCATT | | CATTCA | | variant |
| | AGGTTGCGACAAACGGCAGAAGCTGGAGGAGG | | NNNNN | | |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CaatgttagCAAGAAATCTATG | | | | |
| Var0155 | CATAGATTTCTTGGCCTTTATGCTGTGGTGTTGC | 19697 | ACCTAT | 19889 | Minimal |
| | GCCACGGTGGAGATTTGTCAAATACACATCATT | | CATTCA | | variant |
| | AGGTTGCGACAAACGGCAGAAGCTGGAGGAGG | | NNNNN | | |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CaaatatagCAAGAAATCTATG | | | | |
| Var0156 | CATAGATTTCTTGGCCTTTATGCTGTGGTGTTGC | 19698 | ACCTAT | 19890 | Minimal |
| | GCCACGGTGGAGATTTGTCAAATACACATCATT | | CATTCA | | variant |
| | AGGTTGCGACAAACGGCAGAAGCTGGAGGAGG | | NNNNN | | |
| | AAGGGCCTGAGTCCGAGCAGAAGAACCTGCAG | | NNN | | |
| | GACCTATCATTCANNNNNNNNAAAGTTCTCCCA | | | | |
| | TCACATCAACCGGTGGCGCATTGCCACGAAGCA | | | | |
| | GGCCAATGGGGAGGACATCGATGTCACACAAC | | | | |
| | CaaagCAAGAAATCTATG | | | | |
| Var0157 | CATAGATTTCTTGGCCTTTATGCTGTGGTGTTGC | 19699 | ACCTAT | 19891 | Termination |
| | GCCACGGTggagatttgtcaaatacacataattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0158 | CATAGATTTCTTGGCCTTTATGCTGTGGTGTTGC | 19700 | ACCTAT | 19892 | Termination |
| | GCCACGGTggagatttgtcaaatacacattattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0159 | CATAGATTTCTTGGCCTTTATGCTGTGGTGTTGC | 19701 | ACCTAT | 19893 | Termination |
| | GCCACGGTggagatttgtcaaatacaaatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0160 | CATAGATTTCTTGGCCTTTATGCTGTGGTGTTGC | 19702 | ACCTAT | 19894 | Termination |
| | GCCACGGTggagatttgtcaaatacatatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0161 | CATAGATTTCTTGGCCTTTATGCTGTGGTGTTGC | 19703 | ACCTAT | 19895 | Termination |
| | GCCACGGTggagatttgtcaaatacaaataattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0162 | CATAGATTTCTTGGCCTTTATGCTGTGGTGTTGC | 19704 | ACCTAT | 19896 | Termination |
| | GCCACGGTggagatttgtcaaatacaaattattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0163 | CATAGATTTCTTGGCCTTTATGCTGTGGTGTTGC | 19705 | ACCTAT | 19897 | Termination |
| | GCCACGGTggagatttgtcaaatacatataattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0164 | CATAGATTTCTTGGCCTTTATGCTGTGGTGTTGC | 19706 | ACCTAT | 19898 | Termination |
| | GCCACGGTggagatttgtcaaatacatattattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0165 | CATAGATTTCTTGGCCTTTATGCTGTGGTGTTGC | 19707 | ACCTAT | 19899 | Termination |
| | GCCACGGTggagatttgtcaaataaacatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0166 | CATAGATTTCTTGGCCTTTATGCTGTGGTGTTGC | 19708 | ACCTAT | 19900 | Termination |
| | GCCACGGTggagatttgtcaaatatacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0167 | CATAGATTTCTTGGCCTTTATGCTGTGGTGTTGC | 19709 | ACCTAT | 19901 | Termination |
| | GCCACGGTggagatttgtcaaataaaaatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0168 | CATAGATTTCTTGGCCTTTATGCTGTGGTGTTGC | 19710 | ACCTAT | 19902 | Termination |
| | GCCACGGTggagatttgtcaaataaatatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0169 | CATAGATTTCTTGGCCTTTATGCTGTGGTGTTGC | 19711 | ACCTAT | 19903 | Termination |
| | GCCACGGTggagatttgtcaaatataaatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0170 | CATAGATTTCTTGGCCTTTATGCTGTGGTGTTGC | 19712 | ACCTAT | 19904 | Termination |
| | GCCACGGTggagatttgtcaaatatatatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0171 | CATAGATTTCTTGGCCTTTATGCTGTGGTGTTGC | 19713 | ACCTAT | 19905 | Termination |
| | GCCACGGTggagatttgtaaaatacacatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0172 | CATAGATTTCTTGGCCTTTATGCTGTGGTGTTGC | 19714 | ACCTAT | 19906 | Termination |
| | GCCACGGTggagatttgttaaatacacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0173 | CATAGATTTCTTGGCCTTTATGCTGTGGTGTTGC | 19715 | ACCTAT | 19907 | Termination |
| | GCCACGGTggagatttgtaaaataaacatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0174 | CATAGATTTCTTGGCCTTTATGCTGTGGTGTTGC | 19716 | ACCTAT | 19908 | Termination |
| | GCCACGGTggagatttgtaaaatatacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0175 | CATAGATTTCTTGGCCTTTATGCTGTGGTGTTGC | 19717 | ACCTAT | 19909 | Termination |
| | GCCACGGTggagatttgttaaataaacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0176 | CATAGATTTCTTGGCCTTTATGCTGTGGTGTTGC | 19718 | ACCTAT | 19910 | Termination |
| | GCCACGGTggagatttgttaaatatacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0177 | CATAGATTTCTTGGCCTTTATGCTGTGGTGTTGC | 19719 | ACCTAT | 19911 | Termination |
| | GCCACGGTggagatttatcaaatacacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0178 | CATAGATTTCTTGGCCTTTATGCTGTGGTGTTGC | 19720 | ACCTAT | 19912 | Termination |
| | GCCACGGTggagatttttcaaatacacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0179 | CATAGATTTCTTGGCCTTTATGCTGTGGTGTTGC | 19721 | ACCTAT | 19913 | Termination |
| | GCCACGGTggagatttataaaatacacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0180 | CATAGATTTCTTGGCCTTTATGCTGTGGTGTTGC | 19722 | ACCTAT | 19914 | Termination |
| | GCCACGGTggagatttattaaatacacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0181 | CATAGATTTCTTGGCCTTTATGCTGTGGTGTTGC | 19723 | ACCTAT | 19915 | Termination |
| | GCCACGGTggagatttttaaaatacacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0182 | CATAGATTTCTTGGCCTTTATGCTGTGGTGTTGC | 19724 | ACCTAT | 19916 | Termination |
| | GCCACGGTggagattttttaaatacacatcattaGGTTGCGACA | | CATTCA | | variant |
| | AACGGCAGAAGCTGGAGGAGGAAGGGCCTGAG | | NNNNN | | |
| | TCCGAGCAGAAGAACCTGCAGGACCTATCATTC | | NNN | | |
| | ANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0183 | CATAGATTTCTTGGCCTTTATGCTGTGGTGTTGC | 19725 | ACCTAT | 19917 | Termination |
| | GCCACGGTggagatttgtcaaatacacatccttaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0184 | CATAGATTTCTTGGCCTTTATGCTGTGGTGTTGC | 19726 | ACCTAT | 19918 | Termination |
| | GCCACGGTggagatttgtcaaatacacatcgttaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0185 | CATAGATTTCTTGGCCTTTATGCTGTGGTGTTGC | 19727 | ACCTAT | 19919 | Termination |
| | GCCACGGTggaggtttgtcaaatacacaccattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0186 | CATAGATTTCTTGGCCTTTATGCTGTGGTGTTGC | 19728 | ACCTAT | 19920 | Termination |
| | GCCACGGTggagctttgtcaaatacacagcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0187 | CATAGATTTCTTGGCCTTTATGCTGTGGTGTTGC | 19729 | ACCTAT | 19921 | Termination |
| | GCCACGGTggagattggtcaaatacccatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0188 | CATAGATTTCTTGGCCTTTATGCTGTGGTGTTGC | 19730 | ACCTAT | 19922 | Termination |
| | GCCACGGTggagattcgtcaaatacgcatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0189 | CATAGATTTCTTGGCCTTTATGCTGTGGTGTTGC | 19731 | ACCTAT | 19923 | Termination |
| | GCCACGGTggagatttgtccaatacacatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0190 | CATAGATTTCTTGGCCTTTATGCTGTGGTGTTGC | 19732 | ACCTAT | 19924 | Termination |
| | GCCACGGTggagatttgtcgaatacacatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |
| Var0191 | CATAGATTTCTTGGCCTTTATGCTGTGGTGTTGC | 19733 | ACCTAT | 19925 | Termination |
| | GCCACGGTggagatttgccaaatacacatcattaGGTTGCGAC | | CATTCA | | variant |
| | AAACGGCAGAAGCTGGAGGAGGAAGGGCCTGA | | NNNNN | | |
| | GTCCGAGCAGAAGAACCTGCAGGACCTATCATT | | NNN | | |
| | CANNNNNNNNAAAGTTCTCCCATCACATCAACC | | | | |
| | GGTGGCGCATTGCCACGAAGCAGGCCAATGGG | | | | |
| | GAGGACATCGATGTCACACAACCAAATATAAG | | | | |
| | AATTGTTAGCAAGAAATCTATG | | | | |

The screen methodology is depicted in FIG. 114. Essentially, the ncRNA library is co-transfected into 293T cells with Cas9 mRNA, reverse transcriptase mRNA, and guide RNA targeting EMX1 site. Total nucleic acid including RNA, ssDNA is collected 16 hours after transfection and genomic DNA (gDNA) is harvested 96 hours after transfection. Barcode abundances are measured by in-house high-throughput sequencing of ncRNA, ssDNA template, and genomic EMX1 sites. Barcode counts in ssDNA and gDNA are first normalized to the input ncRNA level from initial ncRNA library. Then the per variant ssDNA template production level or gDNA insertion level are calculated by averaging unique barcodes associated with such variant.

### Results:

Results are shown in the scatter plot of FIG. 115. Genomic insertion level is correlated with the ssDNA template production level (Figure), and the negative control ncRNA exhibits low efficiency in both ssDNA production as well as gDNA insertion. ncRNA variants that exhibit higher activity than the WT R6342S ncRNA can be identified in FIG. 115 as those data points falling in the top right quadrant. Below in Table 31B are listed 13 of the top performing ncRNA variants that show over 1.5-fold higher activity in both levels of gDNA insertion and ssDNA production.

TABLE 31B
top performing ncRNA variants

| Variants with improved performance | Type of variation |
|---|---|
| Var0015 | a1a2 variant |
| Var0020 | a1a2 variant |
| Var0026 | a1a2 variant |
| Var0031 | a1a2 variant |
| Var0044 | msD stem variant |
| Var0060 | msD stem variant |
| Var0061 | msD stem variant |
| Var0062 | msD stem variant |
| Var0065 | msD stem variant |
| Var0066 | msD stem variant |
| Var0070 | msD stem variant |
| Var0071 | msD stem variant |
| Var0076 | msD stem variant |

### Examples: Materials and Methods

Mammalian cell culture HEK293T (ATCC CRF-3216) or 293T-Cas9 (Genecopoeia SL502) cells were cultured in Dulbecco's modified Eagle's medium (DMEM) plus GlutaMAX (Thermo Fisher Scientific), supplemented with 10% (v/v) fetal bovine serum (Gibco). Cells were maintained at 37° C. with 5% C02.

Genomic DNA extraction After incubation, the media was removed from the cells and genomic DNA was extracted by the addition of prepGEM reagent (Thomas Scientific: PUN0050) directly into each well of the tissue culture plate. Lysed cells were transferred to a 96 well PCR plate and incubated at 72° C. for 10 minutes, followed by 95° C. enzyme inactivation step for 2 minutes.

High-throughput DNA sequencing of genomic DNA samples Human EMX1 gene or AAVS1 locus was amplified from genomic DNA samples and sequenced on an Illumina NextSeq. Briefly, amplification primers containing Illumina forward and reverse adapters were used for a first round of PCR (PCR1) amplifying EMX1 targeting site. 25 μl PCR1 reactions were performed with 0.3 μM of each forward and reverse primer, 1 μl of genomic DNA extract and 12.5 μl of KAPA HIFI HOTSTART PCR master mix. PCR reactions were carried out as follows: 95° C. for 1 minute, then 25 cycles of [98° C. for 20 seconds, 65° C. for 15 seconds, and 72° C. for 15 seconds], followed by a final 72° C. extension for 2 minutes. PCR reactions were purified using Ampure XP beads (Beckman Coulter) and eluted in 20 μl H20. Unique Illumina barcoding primer pairs were added to each sample in a secondary PCR reaction (PCR2). Specifically, 25 μl PCR2 reactions were performed with 5 μl of IDT for Illumina UDI primers (Illumina), 1 μl of purified PCR1 reaction, and 12.5 μl of KAPA HIFI HOTSTART PCR master mix. PCR reactions were carried out as follows: 95° C. for 3 minutes, then 10 cycles of [95° C. for 30 seconds, 55° C. for 30 seconds, and 72° C. for 30 seconds], followed by a final 72° C. extension for 5 minutes. PCR2 reactions were purified by SequalPrep Normalization plate kit (Thermo Fisher Scientific) and pooled. Size and purity were evaluated by Tape station D1000 assay (Agilent). DNA concentration was measured by fluorometric quantification (Qubit, Thermo Fisher Scientific) and libraries were sequenced with 30% PhiX sequencing control on an Illumina NextSeq 2000 instrument using P1 or P2 300 cycle kit. Sequencing reads were demultiplexed and alignment of amplicon sequences to a reference sequence was performed using CRISPresso2. CRISPresso2 was run in HDR mode using the desired sequence as the expected allele and precise editing yield was calculated as the number of HDR aligned reads divided by total aligned reads.

Junction qPCR for GFP integration qPCR was used to determine presence of GFP at the EMX1 locus post editing. Primers were designed to flank the 5′ and 3′ junction of GFP insertion at EMX1 locus. Briefly, 25 μl qPCR reactions were performed with 12.5 μl of KAPA HiFi HotStart ReadyMix (Roche), 0.3 μM of each forward and reverse primer, 2 μl of gDNA, 1× of SYBR Green (Thermo Fisher Scientific: 57563), and 1× of Rox Reference Dye (Thermo Fisher Scientific: 54881). qPCR reactions were carried out as follows: 50° C. for 2 minutes, 95° C. for 3 minutes, then 40 cycles of [98° C. for 30 seconds, 67° C. for 30 seconds, and 72° C. for 30 seconds], 72° C. extension for 2 minutes, followed by a melt curve analysis with 95° C. for 15 seconds, 60° C. for 1 minute and 95° C. for 1 second. Delta Ct was obtained by subtracting PCR surrounding junction's Ct value from PCR surrounding cutting's Ct value to relatively quantify GFP insertion at EMX1 locus. Additionally, qPCR samples were run on Tapestation 4200 (Agilent) with High-Sensitivity D1000 Reagents (Agilent) to confirm band size at expectation.

Plasmid based gene editing. Plasmids encoding retrons were synthesized by Twist Bioscience. Plasmids containing Cas12a and TnpB were synthesized by Azenta Life Sciences. The Cas9-mCherry and Cas9-mCherry EMX1 sgRNA plasmids were synthesized by Vector Builder Inc. Quintara Biosciences synthesized deletion variants of RTX3_6342S. For 293T-Cas9 transfections, 25,000 293T-Cas9 cells (Genecopoeia: SL502) were transfected using Lipofectamine 3000 (Thermofisher: L3000001) according to the manufacturer's protocol. Transfected cells were incubated at 37° C. for 72 hrs before lysis in prepGEM reagent (Thomas Scientific: PUN0050) according to the manufacturer's protocol. For K562 cells, A Neon or Lonza Electroporation system was used. 100,000 K562 cells were electroporated in OPTI-MEM (Neon) or SF buffer (Lonza) using settings of 1450V with three 10 ms pulses (Neon) or EH120 (Lonza) and 500 ng of total plasmid DNA (250 ng Retron RT+ncRNA/EMX1 sgRNA fusion, and either 250 ng Cas9-mCherry, 250 ng Cas9-mCherry EMX1 sgRNA, 250 ng Cas12a EMX1, or 250 ng TnpB EMX1. Cells were allowed to incubate at 37° C. for 72 hrs. A 1 μL aliquot of crude lysate was used as template for amplification of the EMX1 target site by PCR. Illumina adaptors and UDIs were added in a second round of PCR before loading onto an Illumina NextSeq. The resulting FASTQ files were used as input for CRISPResso2 which quantified precise editing and indels at the EMX1 locus.

In vitro transcription Plasmid templates were synthesized by Twist Bioscience. T7 promoter was inserted upstream of RT or ncRNA sequences. Plasmid was linearized by Sbf1-HF enzyme (NEB, R3642S) or EcoR V (NEB, R0195L) for 16 hour at 37° C. and the complete linearization was checked by agarose gel electrophoresis. Linearized template was purified by GeneRT PCR purification kit (K0701, Thermo Fisher Scientific) and quantified by Nanodrop. mRNA was synthesized for 16 hour at 37° C. using HiScribe T7 mRNA kit with CleanCap Reagent AG (NEB, E2080S) and ncRNA using HiScribe T7 High Yield RNA synthesis kit (NEB, 2040S) following manufacturer's protocol. After DNAsel treatment, RNA was purified using Qiagen RNeasy midi kit (Qiagen, 75144). RNA concentration was measured by Nanodrop (Thermo Fisher) and purity and size were evaluated by RNA ScreenTape analysis on Tape station (Agilent). RT mRNA was further poly (A) tailed by E. coli Poly(A) polymerase (NEB, M0276) and purified by Qiagen RNeasy midi kit. Tail length was estimated by RNA ScreenTape analysis by comparing before and after poly (A) adenylation.

RNA Transfection by Neon Electroporation system HEK293T (ATCC CRF-3216) or 293T-Cas9 (Genecopoeia SL502) cells were harvested and resuspended in R buffer. 50,000 cells with 1 μl of RNA mixture were electroporated by 10 l Neon tip at 1150 voltage, 20 pulses. Electroporated cells were gently transferred to 96 well plate (VWR) containing prewarmed culture media and cultured for 72 hours before genomic DNA extraction.

RNA Transfection by Lipofectamine MessengerMAX 20,000 HEK293T (ATCC CRF-3216) cells were seeded in 96 well plates. 120,000 cells were seeded in 24 well plates. 16-24 hours post-seeding, cells were transfected with 0.3 μl (or 0.1 μl of Lipofectamine MessengerMax per 100 ng of RNA) of Lipofectamine MessengerMax (Thermo Fisher Scientific) with appropriate amount of RNA mixture. Cells were cultured for 72 hours before genomic DNA extraction.

Circularization of ncRNA The template was designed adopting the strategy and sequences shown in (27). In vitro transcription was performed similarly as described above except that 7.5 mM pf NTPs were used instead of 10 mM concentration. After DNAsel treatment, RNA was purified using Qiagen RNeasy midi kit (Qiagen, 75144). RNA was treated with RNAse R (Biosearch, RNR0725) to enrich circular RNA and purified using Qiagen Rneasy modi kit. RNA concentration was measured by Nanodrop (Thermo Fisher) and purity and size were evaluated by RNA ScreenTape analysis on Tape station (Agilent).

### EXAMPLES: REFERENCES

1. Are we creating a new phenotype? M F, Dohrn. 2021, Neurological Research and Practice.
2. Val50Met hereditary transthyretin amyloidosis: not just a medical problem but a psychosocial burden. J, Gonzalez-Moreno. s.l.: Orphanet Journal of Rare Diseases, 2021, Vol. 16.
3. Synthetic DNA delivery systems. D, Luo. 2000, Nature Biotechnology, pp. 33-37.
4. rAAV immunogenicity, toxicity, and durability in 255 clinical trials: A meta-analysis. W, Shen. 2022, Frontiers in Immunology.
5. Reverse transcriptase associated with the biosynthesis of the branched RNA-linked msDNA in Myxococcus xanthus. S, inouye. s.l.: Cell, 1989, Vol. 56. 709-717.
6. Bacterial retrons function in anti-phage defense. A, Millman. s.l.: Cell, 2020, Vol. 183. 1551-1561.
7. Search-and-replace genome editing without double-strand breaks or donor DNA. A V, anzalone. 2019, Nature, pp. 149-157.
8. Branched RNA covalently linked to the 5′ end of a single-stranded DNA in Stigmatella aurantiaca. T, Furuichi. 1, s.l.: Cell, 1987, Vol. 48. 47-53.
9. Retrons and their applications in genome engineering. A J, Simon. 21, s.l.: Nucleic Acids Research, 2019, Vol. 47.
10. Systematic prediction of genes functionally associated with bacterial retrons and classification of the encoded tripartite system. M R, Mestre. 22, s.l.: Nucleic Acids Research, 2020, Vol. 48.
11. Precise genome editing across kingdoms of life using retron-derived DNA. S C, lopez. 2, s.l. Nature Chemical Biology, 2021, Vol. 18.
12. Precise Gene Editing Preserves Hematopoietic Stem Cell Function following Transient p53-mediated DNA Damage Response. Schiorli, Giulia. s.l.: Cell Stem Cell, 2019, Vol. 24.
13. Initiation of homologous recombination at DNA nicks. Maizels N, Davis L. 14, s.l.: Nucleic Acids Research, 2018, Vol. 46.
14. Cpf1 is a single RNA-guided endonuclease of a class 2 CRISPR-Cas system. Zetsche B, Gootenberg J S, Abudayyeh 00, Slaymaker I M, Makarova K S, Essletzbichler P, Volz S E, Joung J, Van der Oost J, Regev A, Koonin E V, Zhang F. 3, s.l.: Cell, 2015, Vol. 163.
15. Transposase-associated TnpB is a programmable RNA-guided DNA endonuclease. Karvelis, T., Druteika, G., Bigelyte, G. s.l.: Nature, 2021, Vol. 599.
16. Highly accurate protein structure prediction with AlphaFold. Jumper, J., Evans, R., Pritzel, A. s.l.: Nature, 2021, Vol. 596.
17. M-MuLV reverse transcriptase: Selected properties and improved mutants. Oscorbin I P, Filipenko M L. s.l.: Comput Struct Biotechnol J, 2021, Vol. 19.
18. Drag-and-drop genome insertion of large sequences without double-strand DNA cleavage using CRISPR-directed integrases. Yarnall MTN, Ioannidi E O, Schmitt-Ulms C, Krajeski R N et al. 4, s.l.: Nat Biotechnol, 2023, Vol. 41.
19. The attachment of a DNA-binding Sso7D-like protein improves processivity and resistance to inhibitors of M-MuLV reverse transcriptase. Oscorbib I P, Wong P F et al. 24, s.l.: FEBS Lett, 2020, Vol. 594.
20. Engineered CRISPR primer editors with compact, untethered reverse transcriptases. Grünewald, J., Miller, B. R., Szalay, R. N et al. s.l.: Nat Biotechnol, 2023, Vol. 41.
21. Enhancing homology-directed genome editing by active and inactive CRISPR CAS9 using asymmetric donor DNA. Richardson, Christopher D. s.l.: Nature Biotechnology, 2016, Vol. 34.
22. Self-coded 3′ extension of run-off transcripts produces aberrant products during in vitro transcription with T7polymerase. F J Triana-Alonso, M Dabrowski, J Wadzack, K H Nierhaus. 11, s.l.: J Biol Chem, 1995, Vol. 270.
23. Imaging Single mRNA molecules in mammalian cells using an optimized MS2-MCP system. M Vera, E Tutucci, R H Singer. s.l.: Methods Mol Biol, 2019, Vol. 2038.
24. Gene regulation by long non-coding RNAs and its biological functions. L Statello, C-J Guo, L-L Chen, M Huarte. s.l.: Nature reviews molecular cell biology, 2021, Vol. 22.
25. Precise and efficient nucleotide substitution near genomic nick via noncanonical homology-directed repair. Nakajuma K, Zhou Y, Tomita A, Hirade Y, Gurumurthy C B, Nakada S. 2, s.l.: Genome Research, 2018, Vol. 28.
26. Double nicking by RNA-guided CRISPR Cas9 for enhanced genome editing specificity. Ran F A, Hsu P D, Lin C Y, Gootenberg J S, Konermann S, Trevino A E, Scott D A, Inoue A, Matoba, S, Zhang Y, Zhang F. 6, s.l.: Cell, 2013, Vol. 154.
27. Engineering circular RNA for potent and stable translation in eukaryotic cells. Wesselhoeft R A, Kowalski P S, Anderson D G. s.l.: Nature Communications, 2018, Vol. 9.
28. Retrons, msDNA, and the bacterial genome. Lampson B C, Inouye M, Inouye S. s.l.: Cytogenet Genome Res, 2005, Vol. 110.
29. Reverse transcriptase from bacterial retrons require specific secondary structure at the 5′-end of the template for the cDNA priming reaction. Shimamoto T, Hsu M Y, Inouye S, Inoye M. 4, s.l.: JBC, 1993, Vol. 268.
30. In vivo Exon Replacement in the Mouse Atp7b Gene by the Cas9 system. Liu L, Cao J, Chang Q, Xing F, Yan G, Fu L, Wang H, Ma Z, Chen X, Li Y, Li S. 9, s.l.: Human Gene Therapy, 2019, Vol. 30.
31. Retron reverse transcriptase termination and phage defense are dependent on host RNase H1. Palka C, Fishman C B, Bhattarai-Kline S, Myers S A, Shipman S L. 6, s.l.: Nucleic Acids Res, 2022, Vol. 50.
32. Frequent aneuploidy in primary human T cells after CRISPR-Cas9 cleavage. Nahmad A D, Reuveni E, Goldschmidt E et al. s.l.: Nat Biotechnol, 2022, Vol. 10.
33. Deep mutational scanning of S. pyogenes Cas9 reveals important functional domains. Spencer J M, Zhang X. s.l.: Sci. Rep., 2017, Vol. 7.
34. In vivo genome editing improves muscle function in a mouse model of Duchenne muscular dystrophy. Nelson C E, Hakim C H, Ousterout D G et al. 6271, s.l.: Science, 2016, Vol. 351.
35. Clinically relevant correction of Recessive Dystrophic Epidermolysis Bullosa by dual sgRNA CRISPR/Casi-mediated gene editing. Bonafont J, Mencia A, Garcia M et al. 5, s.l.: Mol Ther, 2019, Vol. 27.
36. CRISPR/Cas9-mediated excision of ALS/FTD-causing hexanucleotide repeat expansion in C90RF72 rescues major disease mechanisms in vivo and in vitro. Meij boom K E, Abdallah A, Forham N P et al. 6286, s.l.: Nature Communications, 2022, Vol. 13.
37. Modifications in an Emergency: The role of N1-Methylpseudouridine in COVID-19 Vaccines. Nace K D, Meier J L. 5, s.l.: ACS Cent Sci, 2021, Vol. 7.
38. Genetic engineering of T cells for immunotherapy. Ellis G I, Sheppard N C, Riley J L. s.l. Nature reviews genetics, 2021, Vol. 22.
39. Lipid nanoparticles for mRNA delivery. Hou Z, Zaks T, Langer R, Dong Y. s.l.: Nature Reviews Materials, 2021, Vol. 6.

### Examples: Sequences

Select RT and ncRNA Sequences—Summary

| Description | SEQ ID NO: |
|---|---|
| EMX1 gRNA sequence (nt) | 19109 |
| Eco1 ncRNA (nt) | 19110 |
| AcoI ncRNA (nt) | 19111 |
| RTX_1262 RT (aa) | 1262 |
| RTX_1262 ncRNA (nt) | 15327 |
| RTX3_2042 RT (aa) | 2042 or 19112 |
| RTX_2042 ncRNA (nt) | 19113 |
| RTX_2781 RT (aa) | 2781 |
| RTX_2781 ncRNA (nt) | 16411 |
| RTX_6083v1 RT (aa) | 6083 or 19114 |
| RTX3_6083v1 ncRNA (nt) | 18547 |
| RTX_6342 RT (aa) | 6342 |
| RTX_6342 ncRNA (nt) | 18731 |
| RTX_6342L ncRNA (nt) | 19927 |
| RTX_6342S ncRNA (nt) | 19928 |
| Engineered 6342S ncRNA + HDR template | 19929 |
| Engineered 6342L ncRNA + HDR template | 19930 |
| RTX_6943 RT (aa) | 6943 or 19116 |
| RTX_6943 ncRNA (nt) | 19053 |
| Engineered RTX_6943 ncRNA | 19117 |
| Eco1 RT (nt | 19118 |
| Eco1 ncRNA (nt) | 19119 |
| Eco3 RT (nt) | 19120 |
| Eco3 ncRNA (nt) | 19121 |
| Eco5 RT - (nt) | 19122 |
| Eco5 ncRNA (nt) | 19123 |
| EMX1 - sgRNA modified (nt) | 19124 |
| EMX1 sgRNA unmodified (nt) | 19125 |

Select RT and ncRNA Sequences

EMX1 s.p Cas9 gRNA
(SEQ ID NO: 19109)
GAGTCCGAGCAGAAGAAGAA
Ecol ncRNA + HDR Template
(SEQ ID NO: 19110)
TGATAAGATTCCGTATGCGCACCCTTAGCGAGAGGTTTATCATTAAGGTC
AACCTCTGGATGTTGTTTCGGCATCCTGCATTGAATCTGAGTTACTGTCT
GTTTTCCTACAAACGGCAGAAGCTGGAGGAGGAAGGGCCTGAGTCCGAGC
AGAAGAAAAAGTTCTCCCATCACATCAACCGGTGGCGCATTGCCACGAAG
CAGGCCAATGGGGAGGACATCGATGTCAAGGAAACCCGTTTCTTCTGACG
TAAGGGTGCGCATACGGAATCTTATCA
Acol ncRNA + HDR Template
(SEQ ID NO: 19111)
CCGTAGTGGGAGCCTCAGGCGAGGGTGTGTATCATGCCCGTTCTGCCAAG
ACCCACCAAAGAAGGGCACCGTGGAGGACAAACGGCAGAAGCTGGAGGAG
GAAGGGCCTGAGTCCGAGCAGAAGAAACAGAAGTGCAAAGTTCTCCCATC
ACATCAACCGGTGGCGCATTGCCACGAAGCAGGCCAATGGGGAGGACATC
GATGTCACCTCCACGGTGCAATGCGAAAGCAACTTGAGGCTTTGCTTAGT
ATGAGGCTCCCACTACGG
Retron 1262
RT
(SEQ ID NO: 1262)
MISFSEIKSRNDFADALQIPRSVLTHVLYIAKPESFYESFTIPKKNGEDR
IIMAPKGTLKSIQTKLSKQLVEYRASISQKGQEKSNISHGFEREKSIITN
AQIHRNKRYVINYDLKDFFDSFHFGRVVGFFEKNKHFLLPYEVAVIIAQL
TCYNGRLPQGAPTSPVITNLICEILDYRVLKIAKRYKLDYTRYADDLTFS
TNYSRFLEVFDSFAKELLQEISNSGFTINQSKTRLLYRDSRQEVTGLVVN
KKIGVNREYVKSTRAMAQALYSTGEFTINGIPGTIKQLEGRFGFIDQLDH
YNNVIDDAKHDAYSLNGREKQFQEFLFFKTFFFNEYPLVITEGKTDIRYL
KAALKSLHQKYPELICKEDDGTFRFKISFFRRSKRWKYFFGISKDGADAM
KLLYRFFTGQKGVKNYYRLFAEKYKAVQRNPVIMLFDNEMESKRPLNKFI
SEEVKIPSSEQQLFKEQLYYHLIPGSKTYLMTHPLPPGKTEAEIEDLFPT
EVLGVKLDGKSFSTKDKFDTSKFYGKDIFSSYVYEHWKSIDFSGFIPLLD
KINMLVQNEKKPGLNT
ncRNA
(SEQ ID NO: 15327)
GAGCGTGTGACAAGATTTTGGGCTTGTGTTTCGCAAGCTTTGATTAAAAT
GGTAGATGGATTTGCTATCTATCGTCATTTCCAGTACTGTTATGTTTATG
TTCTGTTTACCCGTATGCACCATCCGCATAATGAGTTGTCACACGCTC
Retron 2042
RT
(SEQ ID NO: 19112 or SEQ ID NO: 2042)
MKDDQYSQWKKYYESRGILPEIQDKLLNYAKIHIDNNTPVIFNFEHLTLL
LGREKNYLSSVVNSPDSHYRKFKIKKRSGGEREITAPYLSLLEMQYWIYR
NILINVKIHYAAHGFAQDKSIITNSRNHLGQKHLLKMDLKDFFPSIKLNR
IIYIFKSLGYPNIIAFYLASICSYKGHLPQGSPTSPILSNIVSITLDNRL
VKFARKMKLRYSRYADDLTFSGDKIPTNYIKYITDIINDEGFEVNDTKTK
LYLKAGKRIVTGISVIGNDPKLPREYKRKLKQELHYIFTYGIGSHMAKKK
IKKINYLYRIIGKVNFWLNIEPDNEYARNAKAKLLLLIDN*
Cognate ncRNA
(SEQ ID NO: 15833)
AGGTGGTTATATTCTAGTATTTATGAAGTGTAGTCGCTTCGATCGTTAAG
GCTGATTTTAACCTCTGCATAATAATATCGGTAGATATTATTATGCACGC
TCCCTTTAGCAGAGCTAAGAATCGCTCACTCAGGCACAAGCTTTGAGGAG
CGACCTCCTCAAAGCTTGTGCCTGAGTGAGAGCTAAAGAAAAGAAAAGTA
GAATAAGCCACCT
Engineered 2042 ncRNA: RTX3_2042
ncRNA + HDR_Template
(SEQ ID NO: 19113)
GTTAAGGTGGTTATATTCTAGTATTTATGAAGTGTAGTCGCTTCGATCGT
TAAGGCTGATTTTAACCTCTGCATAATAATATCGGTAGATATTATTATGC
ACGCTCCCTTTAGCAGAGCTAAGAATCGCTCACTCAGGCACAAGCTTTGA
GGACAAACGGCAGAAGCTGGAGGAGGAAGGGCCTGAGTCCGAGCAGAAGA
ATCCTAAGAGAAAAGTTCTCCCATCACATCAACCGGTGGCGCATTGCCAC
GAAGCAGGCCAATGGGGAGGACATCGATGTCACCTCAAAGCTTGTGCCTG
AGTGAGAGCTAAAGAAAAGAAAAGTAGAATAAGCCACCTTAAC
Retron 2781
RT
(SEQ ID NO: 2781)
MSGRLWKYLTPQQEREFILSLNLLPASCRKRMSGEAQLALLYAISNHVER
HYRKVRIPKKDGKSRRLLVPDGLLKGVQRNILRHCLDGRSVSEYACAYRR
GLSAADNAAPHAGGGKDKLLLKLDIRDFFDSILFWQVYGAAFPGSLFPPA
AAGLLTHLCCCGDRLPQGAPTSPAISNLVMKPFDEFMGAWCRQRQIVYTR
YCDDLTFSGAFDPKAVYHKARHLLEAMGFALNAEKTALRNRGMRQSVTGL
VVNERVRTGVPYRRRIRQEMYYCRKYGVGEHLQAVGRLTGTETQEEAAEA
ERAFLCALLGKIGYVLLLEPENREFLEYREICRGMQAEQEKASCPPACGE
REQKRRGGKEK
Cognate ncRNA
(SEQ ID NO: 16411)
AAGAGCAACTAGATTGAGGCGATTCGCCTCCTTGGAAAAGGGTACTAAGT
TTCTGTCGCACACCAATTTATAAGCTTATAAATTGGTGTGCGACAGAAAT
GAAATAAATAGTAGTTGCTCTT
Retron 6083
RT
(SEQ ID NO: 19114 or SEQ ID NO: 6083)
MSNPQPTRAEIFERIKQSSKQEVILEEMQRLGFWPRSEGQPEVAADLIQR
EGELQRELAELNKKLAVKRNPERALREMRKQRMKDARDKREVTKRAQAQQ
RYDKALLWHEKRASHVAYLGPGVSASLHENSSATQEQGDKGKPKRARDRA
VPDLQRLTLNGLPALISAAQLAESMGVSVAELRFLSFHREVARTNHYHSF
TLPKKTGGERLISAPMPRLKRAQYWVLDNVLAKMPAHDAAHGFLAGRSII
SNAKPHAGQDVVINLDVKDFFPSIAFGRIKGVFRQLGYGESIATVFALLC
SENRAQAWQVDGERLFVGGKARERVLPQGAPTSPMLTNLLCRRMDRRLLG
LAKQLGFVYTRYADDLTFSASGEPARDNVGKLLSRVRWILRDEGFTPHPD
KERVMRKGRRQEVTGLVVNSDTPSVSRETRRRLRAALHRASQPDAASKPA
HWQGHTAQPSQLLGLATFVHQIDPKQGKTLLADAQQLMRSPIDRANDAAK
SASRADAAQQSFRVLAAAGKPPVLADGKNWWQPAPPATPVLEKTDQQRRE
ERQATRRQQAAAAAPPPSSTRRNERPQQAAHEQQGDAQPQNEAPPRFDPD
QYAPPPRNVMTYWAQIAISFFLGSILHNRLITIFAMVAVIALYYMRRQRW
DVFMGILVVATLLGYLVRGMG*
Cognate ncRNA
(SEQ ID NO: 18547)
CCGGAGCAATGAGCAGGCTCTTGCAATCCGGGCGGTGTTTCGCCGCCCTT
GTGAACTGCCGTTTCATGCACCACGGGCGCCGTTTTCACTGTGCCACCCC
AGCCACGGTAGTGCTTTTCCTGAGTGGCTTGCCACAAGCGAAGCAGCACT
ACCGTGGCGGGGTGGCGTCGAGCGAACAGCTCCCGTCCCGTGAGCCCTAC
AGGCTCTTCGACGAGATGCACATTGCTCCG
Engineered 6083 ncRNA: RTX3_6083v1
ncRNA + HDR Template
(SEQ ID NO: 19115)
GCTCCGGAGCAATGAGCAGGCTCTTGCAATCCGGGCGGTGTTTCGCCGCC
CTTGTGAACTGCCGTTTCATGCACCACGGGCGCCGTTTTCACTGTGCCAC
CCCAGCCACGGTAGTGCTACAAACGGCAGAAGCTGGAGGAGGAAGGGCCT
GAGTCCGAGCAGAAGAACTACAAGGTTAAAGTTCTCCCATCACATCAACC
GGTGGCGCATTGCCACGAAGCAGGCCAATGGGGAGGACATCGATGTCAAG
CACTACCGTGGCGGGGTGGCGTCGAGCGAACAGCTCCCGTCCCGTGAGCC
CTACAGGCTCTTCGACGAGATGCACATTGCTCCGGAGC
Retron 6342
RT
(SEQ ID NO: 6342)
MMENQENNMNNRKNYREAVNTMGKKEFTLIKMQEYGFWPKNLPTPYERQE
SETEEQYKERKCLLEKYEKVIDEISKLYEEKDKINLKLRELQKKYDETWD
YERIRLDVSKTIMQESIERRAERKRQRELEKEQRSEEWKKEKENKIVFIG
KGYSSLLYDKETDENKLLLQELPIIKDDKELANFLGIEYKKLRFLVYHRD
VISVDNYHRYTIPKKKGGVRNIAAPKSILKNSQRIILEEILSKIPTSNYS
HGFLKGKSVVSAAKVHVKKPDLLINIDLEDFFPTITFERVRGMFKSFGYS
GYVASMLAMICTYCERMKVEVRGEEKYVKISDRILPQGSPASPMITNIIC
VKLDKRINGLSTKYDFIYTRYADDMSFSFTGDINELSVGSFMGLVSKIVK
EEGFNINKDKTKFLRKNNRQCITGIVINNEEIGVPKKWIKILRAAIYNAN
KVKNSGEILSNKVINEISGMTSWVKSVNEERYKDIINDAMNLINN
Cognate ncRNA
(SEQ ID NO: 18731)
ACATAGATTTCTTGGCCTTTATGCTGTGGTGTTGCGCCACGGTGGAGATT
TGTCAAATACACATCATTAGGTTGCGCGCAATTCGCTACGCTACCAATAC
TGTGGGCATTAAAAATCGAGCCTTCGGTTATGCCACAGTATTGGTGCTAC
GCTGAAGTGTCACAACCAAATATAAGAATTGTTAGCAAGAAATCTATG
RTX_6342S ncRNA
(SEQ ID NO: 19927)
CAUAGAUUUCUUGGCCUUUAUGCUGUGGUGUUGCGCCACGGUGGAGAUUU
GUCAAAUACACAUCAUUAGGUUGCG__place_insert_here__CACA
ACCAAAUAUAAGAAUUGUUAGCAAGAAAUCUAUG
RTX_6342L ncRNA
(SEQ ID NO: 19928)
CAUAGAUUUCUUGGCCUUUAUGCUGUGGUGUUGCGCCACGGUGGAGAUUU
GUCAAAUACACAUCAUUAGGUUGCGCGCAAUUCGCUACGCUACCAAUACU
GUGGGCAU__place_insert_here__AUGCCACAGUAUUGGUGCUAC
GCUGAAGUGUCACAACCAAAUAUAAGAAUUGUUAGCAAGAAAUCUAUG
RTX_6342S ncRNA + HDR
Template Insert
(SEQ ID NO: 19929)
CAUAGAUUUCUUGOCCUUUAUGCUGUGGUGUUGCGCCACOGUGGAGAUUU
GUCAAAUACACAUCAUUAGGUUGCGACAAACGGCAGAAGCTGGAGGAGGA
AGGGCCTGAGTCCGAGCAGAAGAATCGTCAGTGCAAAGTTCTCCCATCAC
ATCAACCGGTGGCGCATTGCCACGAAGCAGGCCAATGGGGAGGACATCGA
TGTCACACAACCAAAUAUAAGAAUUGUUAGCAAGAAAUCUAUG
RTX_6342L ncRNA + HDR
Template Insert
(SEQ ID NO: 19930)
CAUAGAUUUCUUGGCCUUUAUGCUGUGGUGUUGCGCCACGGUGGAGAUUU
GUCAAAUACACAUCAUUAGGUUGCGCGCAAUUCGCUACGCUACCAAUACU
GUGGGCAUACAAACGGCAGAAGCTGGAGGAGGAAGGGCCTGAGTCCGAGC
AGAAGAACGTAATCCGGAAAGTTCTCCCATCACATCAACCGGTGGCGCAT
TGCCACGAAGCAGGCCAATGGGGAGGACATCGATGTCAAUGCCACAGUAU
UGGUGCUACGCUGAAGUGUCACAACCAAAUAUAAGAAUUGUUAGCAAGAA
AUCUAUG
Retron 6943
RT
(SEQ ID NO: 19116 or SEQ ID NO: 6943))
MEESTNYKLLVWGLSVIQPATPNEVLNYLTSTLNDNGLLPDVEKMIHYFE
LLDQLGYIHQVSKRNNLYSLTPRGNERLTPALKRLRDKIRLFMLDNCHSI
SKLGVLASTDTENMGGDSPSLQLRHNLKEVPHPSLSWAAGTLPSSPRQAW
VRIYEQLNIGSMSSDEASTPTTARNAPLSFVGRLGFSLNYYSFNKIDEPL
FNNDGVTAIASCIGISPGLITAMVKSPKRYYRTFNLRKKSGGFRSILAPR
KFIKTIQYWLKDHVLNRLKIHSSCYSYRSGVSIKDNAINHVKKKFVASID
ISDYFGSINKKMVKDCFYKNNIPDHIVNTISGIVTYNDVLPQGAPTSPII
SNAILFEFDEEMTAHALTLDCIYTRYSDDISISSDYKENIAILINIAEAN
LLSAGFTLNRQKQRIASDNSRQVVTGILVNESIRPTRCYRKKIRSAFDHA
LKEQDGSQLTINKLRGYLNYLKSFETYGFKFNEKKYKETLDFLIALKQS*
ncRNA
(SEQ ID NO: 19053)
ACTGATACAGAGAATATGGGCGGTGATTCGCCGTCTTTACAGTTAAGGCA
CAATTTAAAAGAGGTTCCGCACCCAAGCCTGTCTTGGGCTGCAGGGACCC
TGCCCAGCAGCCCAAGACAGGCTTGGGTGCGGATCTACGAGCAATTAAAT
ATTGGTTCGATGTCCAGT
Engineered ncRNA: RTX3_6943
ncRNA + HDR Template
(SEQ ID NO: 19117)
GCGGAGTGCTGGCCTCAACTGATACAGAGAATATGGGCGGTGATTCGCCG
TCTTTACAGTTAAGGCACAATTTAAAAGAGGTTCCGCACCCAAGCCTGTC
TTGGGCTGCACAAACGGCAGAAGCTGGAGGAGGAAGGGCCTGAGTCCGAG
CAGAAGAACGTATGGCCTAAAGTTCTCCCATCACATCAACCGGTGGCGCA
TTGCCACGAAGCAGGCCAATGGGGAGGACATCGATGTCAGCAGCCCAAGA
CAGGCTTGGGTGCGGATCTACGAGCAATTAAATATTGGTTCGATGTCCAG
TGATGAGGCCAGCACCCGC
Eco1 RT [symbol]
Coding sequence only. RNA was produced
from a CRO and proprietary 5′cap, 5′UTR,
3′UTR and encoded polyA of around 120
bases were added. Full substitution with
IN-methyl-pseudouridine)
(SEQ ID NO: 19118)
AUGAAAUCUGCAGAGUAUCUGAAUACGUUCCGCCUUAGGAAUUUGGGCCU
CCCCGUGAUGAACAAUCUCCACGAUAUGAGCAAGGCGACUCGAAUAUCCG
UGGAAACGCUGAGACUGCUCAUCUAUACAGCAGACUUUCGGUACAGGAUC
UACACGGUCGAAAAGAAGGGGCCUGAGAAACGCAUGCGAACAAUUUAUCA
ACCUAGCCGAGAGCUCAAGGCGUUGCAGGGCUGGGUUCUUCGAAACAUCC
UUGACAAACUCUCAUCAUCACCCUUUAGUAUUGGGUUUGAAAAGCACCAA
AGCAUCCUUAACAACGCGACGCCACACAUAGGUGCCAAUUUCAUAUUGAA
CAUCGACUUGGAGGAUUUUUUUCCGAGCCUCACAGCCAAUAAAGUGUUCG
GUGUUUUUCACAGUCUUGGGUACAAUCGCCUUAUUAGUUCCGUUCUUACC
AAGAUUUGUUGUUACAAGAAUCUCUUGCCCCAGGGAGCACCCAGCAGUCC
GAAAUUGGCGAAUUUGAUUUGUUCCAAGCUCGAUUAUCGAAUACAAGGGU
ACGCGGGCAGCCGGGGACUCAUCUAUACCCGCUACGCAGACGAUCUUACG
CUGUCUGCCCAAUCAAUGAAGAAGGUCGUAAAGGCGCGGGAUUUCUUGUU
UUCUAUCAUCCCGUCCGAGGGCUUGGUAAUUAAUUCCAAAAAGACUUGUA
UCUCAGGACCACGAUCUCAGCGAAAAGUGACAGGACUCGUCAUUUCUCAA
GAAAAAGUCGGUAUAGGGAGAGAGAAGUAUAAGGAAAUCCGCGCGAAGAU
CCACCACAUAUUCUGUGGCAAGAGCAGCGAGAUAGAACACGUCCGAGGCU
GGUUGUCCUUCAUACUGAGCGUGGACUCAAAAAGCCACCGCCGGUUGAUC
ACCUAUAUUUCAAAACUGGAAAAGAAAUAUGGAAAGAACCCACUCAACAA
AGCUAAAACACCACCAAAGAAGAAAAGAAAGGUCUGA
EcolncRNA
ncRNA-sgRNA fusion (6 bp
substition in EMX1 gene)
(SEQ ID NO: 19119)
GAAAUGAUAAGAUUCCGUAUGCGCACCCUUAGCGAGAGGUUUAUCAUUAA
GGUCAACCUCUGGAUGUUGUUUCGGCAUCCUGCAUUGAAUCUGAGUUACU
GUCUGUUUUCCUACAAACGGCAGAAGCUGGAGGAGGAAGGGCCUGAGUCC
GAGCAGAAGAAAAAGUUCUCCCAUCACAUCAACCGGUGGCGCAUUGCCAC
GAAGCAGGCCAAUGGGGAGGACAUCGAUGUCAAGGAAACCCGUUUCUUCU
GACGUAAGGGUGCGCAUACGGAAUCUUAUCAGAGUCCGAGCAGAAGAAGA
AGUUUCAGAGCUAUGCUGGAAACAGCAUAGCAAGUUGAAAUAAGGCUAGU
CCGUUAUCAACUUGAAAAAGUGGCACCGAGUCGGUGCUUUUUUUUUUUU
Eco3 RT [symbol]
Coding sequence only. RNA was produced
from a CRO and proprietary 5′cap, 5′UTR,
3′UTR and encoded polyA of around 120
bases were added. Full substitution with
IN-methyl-pseudouridine
(SEQ ID NO: 19120)
AUGCGCAUUUACUCUCUGAUCGACAGCCAAACCUUAAUGACCAAAGGGUU
CGCAUCCGAGGUCAUGAGGAGCCCAGAACCCCCUAAGAAGUGGGACAUUG
CGAAGAAGAAGGGCGGAAUGCGUACGAUAUACCAUCCCUCUUCUAAGGUG
AAGCUGAUACAGUACUGGCUGAUGAACAACGUGUUCUCCAAAUUGCCGAU
GCACAACGCCGCGUACGCUUUCGUGAAGAAUAGAUCUAUCAAGUCUAACG
CACUGCUGCACGCAGAGAGUAAGAACAAAUACUACGUUAAGAUUGACCUG
AAGGACUUCUUUCCAAGCAUCAAGUUCACAGACUUCGAAUAUGCCUUUAC
CCGGUACCGUGACAGAAUAGAGUUCACGACCGAGUACGACAAAGAACUGC
UUCAGCUGAUUAAGACCAUUUGUUUCAUUUCUGACUCUACACUGCCAAUA
GGCUUCCCCACUUCCCCUCUUAUAGCCAAUUUCGUCGCCAGGGAGCUGGA
CGAGAAGCUCACUCAGAAGCUGAACGCUAUAGACAAGCUCAACGCUACGU
ACACUCGCUACGCAGACGACAUAAUCGUGAGCACGAACAUGAAGGGCGCC
UCUAAGCUGAUCUUAGACUGCUUCAAGCGGACCAUGAAGGAAAUCGGACC
CGAUUUCAAGAUCAAUAUCAAGAAGUUCAAAAUAUGCUCUGCCAGUGGCG
GCUCAAUUGUCGUGACGGGCCUUAAGGUCUGUCAUGACUUCCACAUAACU
CUGCACCGGUCUAUGAAGGACAAGAUCCGCCUGCACCUCUCUCUCCUGUC
CAAAGGUAUUCUGAAGGACGAGGACCACAACAAGCUGUCCGGGUACAUCG
CCUACGCUAAGGACAUCGAUCCACACUUCUACACCAAGCUCAAUAGGAAG
UACUUCCAGGAGAUCAAGUGGAUACAAAACCUGCAUAAUAAGGUGGAGCC
ACCAAAGAAGAAAAGAAAGGUCUGA
Eco3 ncRNA
ncRNA-sgRNA fusion (10 bp insertion
in EMX1 gene)
(SEQ ID NO: 19121)
GAAAUGAUAAGAUUCCGUAAGAGCCAAACCUAGCAUUUUAUGGGUUAAUA
GCCCAUCGGGCCAUGAGUCAUGGUUUCGCCUAGUAUUUUAGCUAUGCCCG
UCGUUCAGUUCGCUGAACAAACGGCAGAAGCUGGAGGAGGAAGGGCCUGA
GUCCGAGCAGAAGAAUUACGUCUGCAAAGUUCUCCCAUCACAUCAACCGG
UGGCGCAUUGCCACGAAGCAGGCCAAUGGGGAGGACAUCGAUGUCAUCAG
CGAACUGAUCGACGUGCUCAAGUAGGUUUGGCUCUUACGGAAUCUUAUCA
GAGUCCGAGCAGAAGAAGAAGUUUCAGAGCUAUGCUGGAAACAGCAUAGC
AAGUUGAAAUAAGGCUAGUCCGUUAUCAACUUGAAAAAGUGGCACCGAGU
CGGUGCUUUUUUUUUUUU
Eco5 RT [symbol]
Coding sequence only. RNA was produced
from a CRO and proprietary 5′cap, 5′UTR,
3′UTR and encoded polyA of around 120
bases were added. Full substitution with
IN-methyl-pseudouridine
(SEQ ID NO: 19122)
AUGGACGCUACCAGAACGACUCUCCUUGCAUUGGAUCUCUUCGGAUCUCC
AGGUUGGUCCGCCGAUAAAGAAAUUCAGAGGCUUCAUGCGCUCAGUAAUC
AUGCUGGAAGGCAUUACAGAAGGAUUAUAUUAAGUAAAAGGCACGGCGGA
CAGCGUCUUGUGCUUGCACCUGAUUACUUGUUAAAGACCGUUCAGCGCAA
CAUUUUGAAGAACGUUUUGAGUCAAUUUCCACUGUCACCAUUUGCUACAG
CCUACAGACCGGGAUGCCCAAUCGUGUCUAACGCGCAGCCACACUGCCAA
CAGCCACAGAUCUUGAAACUCGAUAUAGAAAACUUCUUCGAUUCUAUUAG
UUGGUUGCAGGUGUGGCGGGUGUUUCGCCAGGCCCAGUUGCCCCGAAAUG
UCGUAACGAUGCUCACUUGGAUAUGUUGUUAUAACGACGCACUUCCGCAG
GGUGCCCCUACAUCCCCUGCAAUUUCCAAUCUCGUCAUGAGAAGGUUUGA
UGAACGGAUUGGAGAAUGGUGUCAGGCUCGAGGGAUUACCUACACUCGCU
ACUGCGAUGACAUGACGUUUAGUGGACACUUCAAUGCAAGGCAGGUCAAG
AAUAAAGUCUGCGGUCUCUUAGCUGAGCUGGGCCUUUCCCUGAAUAAACG
GAAAGGCUGCCUCAUAGCGGCUUGUAAGCGCCAGCAAGUCACCGGCAUUG
UUGUGAAUCACAAGCCACAGCUUGCCCGAGAAGCCAGGCGUGCCCUGCGU
CAGGAAGUGCACCUGUGCCAGAAAUAUGGAGUUAUCUCUCAUCUCUCACA
UAGAGGUGAACUGGAUCCUAGCGGAGAUCUGCACGCUCAGGCGACAGCGU
AUCUCUAUGCACUCCAGGGGAGAAUUAACUGGCUUCUUCAAAUUAACCCU
GAGGAUGAGGCGUUUCAACAGGCCCGGGAGUCCGUUAAGAGGAUGUUAGU
UGCCUGGCCACCAAAGAAGAAAAGAAAGGUCUGA
Eco5 ncRNA [symbol]
ncRNA-sgRNA fusion (10 bp insertion
in EMX1 gene)
(SEQ ID NO: 19123)
GAAAUGAUAAGAUUCCGUACGCCAGCAGUGGCAAUAGCGUUUCCGGCCUU
UUGUGCCGGGAGGGUCGGCGAGUCGCUGACUUAACGCCAGUAGUAUGUCC
AUAUACCCAAAGUCGCUUCAUUGUAACAAACGGCAGAAGCUGGAGGAGGA
AGGGCCUGAGUCCGAGCAGAAGAAUCCUUCGAGCAAAGUUCUCCCAUCAC
AUCAACCGGUGGCGCAUUGCCACGAAGCAGGCCAAUGGGGAGGACAUCGA
UGUCAUACAGUUACGCGCCUUCGGGAUGGUUUAAUGGUAUUGCCGCUGUU
GGCGUACGGAAUCUUAUCAGAGUCCGAGCAGAAGAAGAAGUUUCAGAGCU
AUGCUGGAAACAGCAUAGCAAGUUGAAAUAAGGCUAGUCCGUUAUCAACU
UGAAAAAGUGGCACCGAGUCGGUGCUUUUUUUUUUUU
EMX1 sgRNA modified [symbol]
Contains chemical modifications from
Synthego: 2′-F, 2′-O-methyl,
phosphorothioates
(SEQ ID NO: 19124)
GAGUCCGAGCAGAAGAAGAAGUUUCAGAGCUAUGCUGGAAACAGCAUAGC
AAGUUGAAAUAAGGCUAGUCCGUUAUCAACUUGAAAAAGUGGCACCGAGU
CGGUGCUUUUU
EMX1 sgRNA unmodified [symbol]
No modifications
(SEQ ID NO: 19125)
GAGUCCGAGCAGAAGAAGAAGUUUCAGAGCUAUGCUGGAAACAGCAUAGC
AAGUUGAAAUAAGGCUAGUCCGUUAUCAACUUGAAAAAGUGGCACCGAGU
CGGUGCUUUUU

### RANA Sequences—Summary

| | | SEQ ID |
|---|---|---|
| Sequence Name | Description | NO: |
| Eco1 RT | Coding sequence only. RNA was produced from a CRO and | 19126 |
| | proprietary 5′ cap, 5′ UTR, 3′ UTR and encoded poly A of around 120 | |
| | bases were added. Full substitution with 1N-methyl-pseudouridine | |
| Eco1 ncRNA-sgRNA | ncRNA-sgRNA fusion (6 bp substition in EMX1 gene) | 19127 |
| Eco3 RT | Coding sequence only. RNA was produced from a CRO and | 19128 |
| | proprietary 5′ cap, 5′ UTR, 3′ UTR and encoded poly A of around 120 | |
| | bases were added. Full substitution with 1N-methyl-pseudouridine | |
| Eco3 ncRNA-sgRNA | ncRNA-sgRNA fusion (10 bp insertion in EMX1 gene) | 19129 |
| Eco5 RT | Coding sequence only. RNA was produced from a CRO and | 19130 |
| | proprietary 5′ cap, 5′ UTR, 3′ UTR and encoded poly A of around 120 | |
| | bases were added. Full substitution with 1N-methyl-pseudouridine | |
| Eco5 ncRNA-sgRNA | ncRNA-sgRNA fusion (10 bp insertion in EMX1 gene) | 19131 |
| Cas9 | All U are N-1-methylpseudouridine. | 19132 |
| EMX1 sgRNA modified | Contains chemical modifications from Synthego: 2′-F, 2′-O-methyl, | 19133 |
| | phosphorothioates | |
| EMX1 sgRNA unmodified | No modifications | 19134 |
| Eco3 ncRNA | Eco3 ncRNA only with 10 bp insertion | 19135 |
| Eco3 ncRNA-MS2 | Eco3 ncRNA only with 10 bp insertion and 3′ MS2 stem loop | 19136 |
| Aco1 RT | Aco1 RT RNA sequence including 5′ and 3′ UTRs | 19137 |
| Aco1 ncRNA-sgRNA_50 | Aco1 ncRNA-sgRNA with 50 bp insertion | 19138 |
| Aco1 sgRNA-ncRNA_50 | Aco1 sgRNA-ncRNA with 50 bp insertion | 19139 |
| Eco3_EMX1 gRNA_ncRNA_25 | Eco3 ncRNA containing 25 bp insertion in EMX1 gene fused to | 19140 |
| | EMX1 sgRNA at 5′ end | |
| Eco3_ncRNA only | Eco3 ncRNA containing 25 bp insertion in EMX1 gene | 19141 |
| Eco3_ncRNA_AAVS1 gRNA_25 | Eco3 ncRNA containing 25 bp insertion in AAVS1 gene fused to | 19142 |
| | AAVS1 sgRNA at 3′ end | |
| Eco3_ncRNA_EMX1 gRNA_25 | Eco3 ncRNA containing 25 bp insertion in EMX1 gene fused to | 19143 |
| | EMX1 sgRNA at 3′ end | |
| Eco3_ncRNA_EMX1 gRNA_50 | Eco3 ncRNA containing 50 bp insertion in EMX1 gene fused to | 19144 |
| | EMX1 sgRNA at 3′ end | |
| Eco3_ncRNA_EMX1 gRNA_75 | Eco3 ncRNA containing 75 bp insertion in EMX1 gene fused to | 19145 |
| | EMX1 sgRNA at 3′ end | |
| Eco3_ncRNA_EMX1 gRNA_100 | Eco3 ncRNA containing 100 bp insertion in EMX1 gene fused to | 19146 |
| | EMX1 sgRNA at 3′ end | |
| Eco3_ncRNA_EMX1 gRNA_GFP | Eco3 ncRNA containing GFP gene insertion in EMX1 gene fused to | 19147 |
| gene | EMX1 sgRNA at 3′ end. Entire GFP cassette is in antisense | |
| | orientation and contains mini EF1a promoter and beta globin polyA | |
| | signal. | |
| Eco3_NT_ncRNA_EMX gRNA_25 | Eco3 ncRNA containing 25 bp insertion (on opposite strand of | 19148 |
| | sgRNA cut) in EMX1 gene fused to EMX1 sgRNA at 3′ end | |
| Aco1_EMX1 gRNA_ncRNA_25 | Aco1 ncRNA containing 25 bp insertion in EMX1 gene fused to | 19149 |
| | EMX1 sgRNA at 5′ end | |
| Aco1_ncRNA only | Aco1 ncRNA containing 25 bp insertion in EMX1 gene | 19150 |
| Aco1_ncRNA_AAVS1 gRNA_25 | Aco1 ncRNA containing 25 bp insertion in AAVS1 gene fused to | 19151 |
| | AAVS1 sgRNA at 3′ end | |
| Aco1_ncRNA_EMX1 gRNA_25 | Aco1 ncRNA containing 25 bp insertion in EMX1 gene fused to | 19152 |
| | EMX1 sgRNA at 3′ end | |
| Aco1_ncRNA_EMX1 gRNA_50 | Aco1 ncRNA containing 50 bp insertion in EMX1 gene fused to | 19153 |
| | EMX1 sgRNA at 3′ end | |
| Aco1_ncRNA_EMX1 gRNA_75 | Aco1 ncRNA containing 75 bp insertion in EMX1 gene fused to | 19154 |
| | EMX1 sgRNA at 3′ end | |
| Aco1_ncRNA_EMX1 gRNA_100 | Aco1 ncRNA containing 100 bp insertion in EMX1 gene fused to | 19155 |
| | EMX1 sgRNA at 3′ end | |
| Aco1_ncRNA_EMX1 gRNA_GFP | Aco1 ncRNA containing GFP gene insertion in EMX1 gene fused to | 19156 |
| gene | EMX1 sgRNA at 3′ end. Entire GFP cassette is in antisense | |
| | orientation and contains mini EF1a promoter and bcta globin poly A | |
| | signal. | |
| Aco1_NT_ncRNA_EMX1 gRNA_25 | Aco1 ncRNA containing 25 bp insertion (on opposite strand of | 19157 |
| | sgRNA cut) in EMX1 gene fused to EMX1 sgRNA at 3′ end | |
| R2042_EMX1 gRNA_ncRNA_25 | R2042 ncRNA containing 25 bp insertion in EMX1 gene fused to | 19158 |
| | EMX1 sgRNA at 5′ end | |
| R2042_ncRNA only_25 | R2042 ncRNA containing 25 bp insertion in EMX1 genc | 19159 |
| R2042_ncRNA_AAVS1 gRNA_25 | R2042 ncRNA containing 25 bp insertion in EMX1 gene fused to | 19160 |
| | AAVS1 sgRNA at 3′ cnd | |
| R2042_ncRNA_EMX1 gRNA_25 | R2042 ncRNA containing 25 bp insertion in EMX1 gene fused to | 19161 |
| | EMX1 sgRNA at 3′ end | |
| R2042_ncRNA_EMX1 gRNA_50 | R2042 ncRNA containing 50 bp insertion in EMX1 gene fused to | 19162 |
| | EMX1 sgRNA at 3′ end | |
| R2042_ncRNA_EMX1 gRNA_75 | R2042 ncRNA containing 75 bp insertion in EMX1 gene fused to | 19163 |
| | EMX1 sgRNA at 3′ end | |
| R2042_ncRNA_EMX1 gRNA_100 | R2042 ncRNA containing 100 bp insertion in EMX1 gene fused to | 19164 |
| | EMX1 sgRNA at 3′ end | |
| R2042_ncRNA_NT_EMX1 | R2042 ncRNA containing 25 bp insertion (on opposite strand of | 19165 |
| gRNA_25_dual guides | sgRNA cut) in EMX1 gene fused to EMX1 sgRNA at 3′ end | |
| R2042_RT | R2042 RT mRNA sequence, coding sequence only | 19166 |
| R6943_RT | R6943 RT mRNA sequence, coding sequence only | 19167 |
| R6943_ncRNA_EMX1gRNA_GFP | R6943 ncRNA containing GFP gene insertion in EMX1 gene fused | 19168 |
| gene | to EMX1 sgRNA at 3′ end. Entire GFP cassette is in antisense | |
| | orientation and contains mini EF1a promoter and beta globin poly A | |
| | signal. | |
| R6943_ncRNA_EMX1gRNA_100bp | R6943 ncRNA containing 100 bp insertion in EMX1 gene fused to | 19169 |
| | EMX1 sgRNA at 3′ end | |
| R6943_ncRNA_EMX1gRNA_75bp | R6943 ncRNA containing 75 bp insertion in EMX1 gene fused to | 19170 |
| | EMX1 sgRNA at 3′ end | |
| R6943_ncRNA_EMX1gRNA_50bp | R6943 ncRNA containing 50 bp insertion in EMX1 gene fused to | 19171 |
| | EMX1 sgRNA at 3′ end | |
| R6943_ncRNA_EMX1gRNA_25bp | R6943 ncRNA containing 25 bp insertion in EMX1 gene fused to | 19172 |
| | EMX1 sgRNA at 3′ end | |
| R6943_ncRNA_AAVS1 | R6943 ncRNA containing 25 bp insertion in AAVS1 gene fused to | 19173 |
| gRNA_25bp | AAVS1 sgRNA at 3′ end | |
| R6943_ncRNA only_25bp | R6943 ncRNA containing 25 bp insertion in EMX1 gene | 19174 |
| AAVS1 sgRNA | Contains chemical modifications from Synthego: 2′-F, 2′-O-methyl, | 19175 |
| | phosphorothioates | |
| R1262_RT-1 (R1262 RT) | R1262 retron RT mRNA | 19176 |
| R1262_nc-5 (R1262_ncRNA- | R1262 ncRNA containing 505 bp insertion in EMX1 site | 19177 |
| EMX1_505) | | |
| R1262_nc-3 (R1262_ncRNA- | R1262 ncRNA containing 305 bp insertion in EMX1 site | 19178 |
| EMX1_305) | | |
| R1262_nc-1 (R1262_ncRNA- | R1262 ncRNA containing 25 bp insertion in EMX1 site | 19179 |
| EMX1_25) | | |
| R1262_nc-4 (R1262_ncRNA- | R1262 ncRNA containing 405 bp insertion in EMX1 site | 19180 |
| EMX1_405) | | |
| R1262_nc-2 (R1262_ncRNA- | R1262 ncRNA containing 205 bp insertion in EMX1 site | 19181 |
| EMX1_205) | | |
| R1262_nc-19 (R1262_ncRNA- | R1262 ncRNA containing 205 bp insertion in EMX1 site combined | 19182 |
| sgEMX1_205) | with EMX1 sgRNA at 3′ end | |
| R1262_nc-20 (R1262_sgEMX1- | R1262 ncRNA containing 205 bp insertion in EMX1 site combined | 19183 |
| ncRNA_205) | with EMX1 sgRNA at 5′ end | |
| R1262_nc-13 (R1262_ncRNA- | R1262 ncRNA containing 25 bp insertion in AAVS1 site | 19184 |
| AAVS1_25) | | |
| R1262_nc-14 (R1262_ncRNA- | R1262 ncRNA containing 205 bp insertion in AAVS1 site | 19185 |
| AAVS1_205) | | |
| R1262_nc-15 (R1262_ncRNA- | R1262 ncRNA containing 505 bp insertion in AAVS1 site | 19186 |
| AAVS1_505) | | |
| R1262_nc-18 (R1262_ncRNA- | R1262 ncRNA containing 505 bp insertion in AAVS1 site and MS2 | 19187 |
| AAVS1-MS2_505) | stem loop at 3′ end | |
| R1262_nc-17 (R1262_ncRNA- | R1262 ncRNA containing 205 bp insertion in AAVS1 site and MS2 | 19188 |
| AAVS1-MS2 205) | stem loop at 3′ end | |
| R1262_nc-16 (R1262_ncRNA- | R1262 ncRNA containing 25 bp insertion in AAVS1 site and MS2 | 19189 |
| AAVS1-MS2_25) | stem loop at 3′ end | |
| R1262_nc-9 (R1262_ncRNA-EMX1- | R1262 ncRNA containing 305 bp insertion in EMX1 site and MS2 | 19190 |
| MS2_305) | stem loop at 3′ end | |
| R1262_nc-7 (R1262_ncRNA-EMX1- | R1262 ncRNA containing 25 bp insertion in EMX1 site and MS2 stem | 19191 |
| MS2_25) | loop at 3′ end | |
| R1262_nc-6 (R1262_ncRNA- | R1262 ncRNA containing P2A-GFP insertion in EMX1 site | 19192 |
| EMX1_P2A-GFP) | | |
| R1262_nc-22 (R1262_sgAAVS1- | R1262 ncRNA containing 205 bp insertion in AAVS1 site combined | 19193 |
| ncRNA_205) | with sgAAVS1 at 5′ end | |
| R1262_nc-8 (R1262_ncRNA-EMX1- | R1262 ncRNA containing 205 bp insertion in EMX1 site and MS2 | 19194 |
| MS2_205) | stem loop at 3′ end | |
| R1262_nc-21 (R1262_ncRNA- | R1262 ncRNA containing 205 bp insertion in AAVS1 site combined | 19195 |
| sgAAVS1_205) | with sgAAVS1 at 3′ end | |
| R1262_nc-10 (R1262_ncRNA- | R1262 ncRNA containing 405 bp insertion in EMX1 site and MS2 | 19196 |
| EMX1-MS2_405) | stem loop at 3′ end | |
| R1262_nc-11 (R1262_ncRNA- | R1262 ncRNA containing 505 bp insertion in EMX1 site and MS2 | 19197 |
| EMX1-MS2_505) | stem loop at 3′ end | |
| R1262_nc-12 (R1262_ncRNA- | R1262 ncRNA containing P2A-GFP insertion in EMX1 site and MS2 | 19198 |
| EMX1-MS2_P2A-GFP) | stem loop at 3′ end | |
| R1262_nc-23 (R1262_ncRNA- | Same as R1262_nc-6 but with longer homology arms | 19199 |
| EMX1_P2A-GFP_LongHA) | | |
| R1262_nc-24 (R1262_ncRNA- | Same as R1262_nc-12 but with longer homology arms | 19200 |
| EMX1-MS2_P2A-GFP_LongHA) | | |
| 6083 v1 RT | 6083 retron RT mRNA | 19201 |
| 6083 v1_ncRNA_AAVS1 | R6083 retron containing 25 bp insertion at AAVS1 site | 19202 |
| gRNA_25bp | | |
| 6083 v1_ncRNA only_25bp | R6083 retron containing 25 bp insertion at EMX1 site | 19203 |
| 6083 v1_ncRNA EMX1 | R6083 retron containing 25 bp insertion at EMX1 site and sgEMX1 at | 19204 |
| gRNA_25bp | 3′ end | |
| 6083 v1_ncRNA_EMX1 gRNA_GFP | R6083 retron containing GFP gene insertion at EMX1 site and | 19205 |
| gene | sgEMX1 at 3′ end | |
| 6083 v1_ncRNA_EMX1 | R6083 retron containing 50 bp insertion at EMX1 site and sgEMX1 at | 19206 |
| gRNA_50bp | 3′ end | |
| 6083 v1_ncRNA_EMX1 | R6083 retron containing 100 bp insertion at EMX1 site and sgEMX1 | 19207 |
| gRNA_100bp | at 3′ end | |
| 6083 v1_ncRNA_EMX1 | R6083 retron containing 75 bp insertion at EMX1 site and sgEMX1 at | 19208 |
| gRNA_75bp | 3′ end | |
| Aco1_ncRNA-EMX1_100 | Aco1 ncRNA containing 100 bp insertion in EMX1 gene | 19209 |
| Aco1_ncRNA-EMX1_205 | Aco1 ncRNA containing 205 bp insertion in EMX1 gene | 19210 |
| Eco3 ncRNA-EMX1_205 | Eco3 ncRNA containing 205 bp insertion in EMX1 gene | 19211 |

| Sequence | | |
|---|---|---|
| Name | Description | Sequence |
| Eco1 RT | Coding sequence only. RNA | AUGAAAUCUGCAGAGUAUCUGAAUACGUUCCGCCUUAGGAAUU |
| | was produced from a CRO and | UGGGCCUCCCCGUGAUGAACAAUCUCCACGAUAUGAGCAAGGCG |
| | proprietary 5′ cap, 5′ UTR, 3′ | ACUCGAAUAUCCGUGGAAACGCUGAGACUGCUCAUCUAUACAGC |
| | UTR and encoded polyA of | AGACUUUCGGUACAGGAUCUACACGGUCGAAAAGAAGGGGCCU |
| | around 120 bases were added. | GAGAAACGCAUGCGAACAAUUUAUCAACCUAGCCGAGAGCUCAA |
| | Full substitution with 1N- | GGCGUUGCAGGGCUGGGUUCUUCGAAACAUCCUUGACAAACUCU |
| | methyl-pseudouridine | CAUCAUCACCCUUUAGUAUUGGGUUUGAAAAGCACCAAAGCAUC |
| | | CUUAACAACGCGACGCCACACAUAGGUGCCAAUUUCAUAUUGAA |
| | | CAUCGACUUGGAGGAUUUUUUUCCGAGCCUCACAGCCAAUAAAG |
| | | UGUUCGGUGUUUUUCACAGUCUUGGGUACAAUCGCCUUAUUAG |
| | | UUCCGUUCUUACCAAGAUUUGUUGUUACAAGAAUCUCUUGCCCC |
| | | AGGGAGCACCCAGCAGUCCGAAAUUGGCGAAUUUGAUUUGUUCC |
| | | AAGCUCGAUUAUCGAAUACAAGGGUACGCGGGCAGCCGGGGACU |
| | | CAUCUAUACCCGCUACGCAGACGAUCUUACGCUGUCUGCCCAAU |
| | | CAAUGAAGAAGGUCGUAAAGGCGCGGGAUUUCUUGUUUUCUAU |
| | | CAUCCCGUCCGAGGGCUUGGUAAUUAAUUCCAAAAAGACUUGUA |
| | | UCUCAGGACCACGAUCUCAGCGAAAAGUGACAGGACUCGUCAUU |
| | | UCUCAAGAAAAAGUCGGUAUAGGGAGAGAGAAGUAUAAGGAAA |
| | | UCCGCGCGAAGAUCCACCACAUAUUCUGUGGCAAGAGCAGCGAG |
| | | AUAGAACACGUCCGAGGCUGGUUGUCCUUCAUACUGAGCGUGGA |
| | | CUCAAAAAGCCACCGCCGGUUGAUCACCUAUAUUUCAAAACUGG |
| | | AAAAGAAAUAUGGAAAGAACCCACUCAACAAAGCUAAAACACCA |
| | | CCAAAGAAGAAAAGAAAGGUCUGA (SEQ ID NO: 19126) |
| Eco1 ncRNA- | ncRNA-sgRNA fusion (6 bp | GAAAUGAUAAGAUUCCGUAUGCGCACCCUUAGCGAGAGGUUUA |
| sgRNA | substition in EMX1 gene) | UCAUUAAGGUCAACCUCUGGAUGUUGUUUCGGCAUCCUGCAUUG |
| | | AAUCUGAGUUACUGUCUGUUUUCCUACAAACGGCAGAAGCUGG |
| | | AGGAGGAAGGGCCUGAGUCCGAGCAGAAGAAAAAGUUCUCCCA |
| | | UCACAUCAACCGGUGGCGCAUUGCCACGAAGCAGGCCAAUGGGG |
| | | AGGACAUCGAUGUCAAGGAAACCCGUUUCUUCUGACGUAAGGG |
| | | UGCGCAUACGGAAUCUUAUCAGAGUCCGAGCAGAAGAAGAAGU |
| | | UUCAGAGCUAUGCUGGAAACAGCAUAGCAAGUUGAAAUAAGGC |
| | | UAGUCCGUUAUCAACUUGAAAAAGUGGCACCGAGUCGGUGCUU |
| | | UUUUUUUUUU (SEQ ID NO: 19127) |
| Eco3 RT | Coding sequence only. RNA | AUGCGCAUUUACUCUCUGAUCGACAGCCAAACCUUAAUGACCAA |
| | was produced from a CRO and | AGGGUUCGCAUCCGAGGUCAUGAGGAGCCCAGAACCCCCUAAGA |
| | proprietary 5′ cap, 5′ UTR, 3′ | AGUGGGACAUUGCGAAGAAGAAGGGCGGAAUGCGUACGAUAUA |
| | UTR and encoded polyA of | CCAUCCCUCUUCUAAGGUGAAGCUGAUACAGUACUGGCUGAUGA |
| | around 120 bases were added. | ACAACGUGUUCUCCAAAUUGCCGAUGCACAACGCCGCGUACGCU |
| | Full substitution with 1N- | UUCGUGAAGAAUAGAUCUAUCAAGUCUAACGCACUGCUGCACGC |
| | methyl-pseudouridine | AGAGAGUAAGAACAAAUACUACGUUAAGAUUGACCUGAAGGAC |
| | | UUCUUUCCAAGCAUCAAGUUCACAGACUUCGAAUAUGCCUUUAC |
| | | CCGGUACCGUGACAGAAUAGAGUUCACGACCGAGUACGACAAAG |
| | | AACUGCUUCAGCUGAUUAAGACCAUUUGUUUCAUUUCUGACUCU |
| | | ACACUGCCAAUAGGCUUCCCCACUUCCCCUCUUAUAGCCAAUUU |
| | | CGUCGCCAGGGAGCUGGACGAGAAGCUCACUCAGAAGCUGAACG |
| | | CUAUAGACAAGCUCAACGCUACGUACACUCGCUACGCAGACGAC |
| | | AUAAUCGUGAGCACGAACAUGAAGGGCGCCUCUAAGCUGAUCUU |
| | | AGACUGCUUCAAGCGGACCAUGAAGGAAAUCGGACCCGAUUUCA |
| | | AGAUCAAUAUCAAGAAGUUCAAAAUAUGCUCUGCCAGUGGCGG |
| | | CUCAAUUGUCGUGACGGGCCUUAAGGUCUGUCAUGACUUCCACA |
| | | UAACUCUGCACCGGUCUAUGAAGGACAAGAUCCGCCUGCACCUC |
| | | UCUCUCCUGUCCAAAGGUAUUCUGAAGGACGAGGACCACAACAA |
| | | GCUGUCCGGGUACAUCGCCUACGCUAAGGACAUCGAUCCACACU |
| | | UCUACACCAAGCUCAAUAGGAAGUACUUCCAGGAGAUCAAGUGG |
| | | AUACAAAACCUGCAUAAUAAGGUGGAGCCACCAAAGAAGAAAA |
| | | GAAAGGUCUGA (SEQ ID NO: 19128) |
| Eco3 ncRNA- | ncRNA-sgRNA fusion (10 bp | GAAAUGAUAAGAUUCCGUAAGAGCCAAACCUAGCAUUUUAUGG |
| sgRNA | insertion in EMX1 gene) | GUUAAUAGCCCAUCGGGCCAUGAGUCAUGGUUUCGCCUAGUAUU |
| | | UUAGCUAUGCCCGUCGUUCAGUUCGCUGAACAAACGGCAGAAGC |
| | | UGGAGGAGGAAGGGCCUGAGUCCGAGCAGAAGAAUUACGUCUG |
| | | CAAAGUUCUCCCAUCACAUCAACCGGUGGCGCAUUGCCACGAAG |
| | | CAGGCCAAUGGGGAGGACAUCGAUGUCAUCAGCGAACUGAUCGA |
| | | CGUGCUCAAGUAGGUUUGGCUCUUACGGAAUCUUAUCAGAGUCC |
| | | GAGCAGAAGAAGAAGUUUCAGAGCUAUGCUGGAAACAGCAUAG |
| | | CAAGUUGAAAUAAGGCUAGUCCGUUAUCAACUUGAAAAAGUGG |
| | | CACCGAGUCGGUGCUUUUUUUUUUUU (SEQ ID NO: 19129) |
| Eco5 RT | Coding sequence only. RNA | AUGGACGCUACCAGAACGACUCUCCUUGCAUUGGAUCUCUUCGG |
| | was produced from a CRO and | AUCUCCAGGUUGGUCCGCCGAUAAAGAAAUUCAGAGGCUUCAUG |
| | proprietary 5′ cap, 5′ UTR, 3′ | CGCUCAGUAAUCAUGCUGGAAGGCAUUACAGAAGGAUUAUAUU |
| | UTR and encoded polyA of | AAGUAAAAGGCACGGCGGACAGCGUCUUGUGCUUGCACCUGAUU |
| | around 120 bases were added. | ACUUGUUAAAGACCGUUCAGCGCAACAUUUUGAAGAACGUUUU |
| | Full substitution with 1N- | GAGUCAAUUUCCACUGUCACCAUUUGCUACAGCCUACAGACCGG |
| | methyl-pseudouridine | GAUGCCCAAUCGUGUCUAACGCGCAGCCACACUGCCAACAGCCA |
| | | CAGAUCUUGAAACUCGAUAUAGAAAACUUCUUCGAUUCUAUUA |
| | | GUUGGUUGCAGGUGUGGCGGGUGUUUCGCCAGGCCCAGUUGCCC |
| | | CGAAAUGUCGUAACGAUGCUCACUUGGAUAUGUUGUUAUAACG |
| | | ACGCACUUCCGCAGGGUGCCCCUACAUCCCCUGCAAUUUCCAAU |
| | | CUCGUCAUGAGAAGGUUUGAUGAACGGAUUGGAGAAUGGUGUC |
| | | AGGCUCGAGGGAUUACCUACACUCGCUACUGCGAUGACAUGACG |
| | | UUUAGUGGACACUUCAAUGCAAGGCAGGUCAAGAAUAAAGUCU |
| | | GCGGUCUCUUAGCUGAGCUGGGCCUUUCCCUGAAUAAACGGAAA |
| | | GGCUGCCUCAUAGCGGCUUGUAAGCGCCAGCAAGUCACCGGCAU |
| | | UGUUGUGAAUCACAAGCCACAGCUUGCCCGAGAAGCCAGGCGUG |
| | | CCCUGCGUCAGGAAGUGCACCUGUGCCAGAAAUAUGGAGUUAUC |
| | | UCUCAUCUCUCACAUAGAGGUGAACUGGAUCCUAGCGGAGAUCU |
| | | GCACGCUCAGGCGACAGCGUAUCUCUAUGCACUCCAGGGGAGAA |
| | | UUAACUGGCUUCUUCAAAUUAACCCUGAGGAUGAGGCGUUUCA |
| | | ACAGGCCCGGGAGUCCGUUAAGAGGAUGUUAGUUGCCUGGCCAC |
| | | CAAAGAAGAAAAGAAAGGUCUGA (SEQ ID NO: 19130) |
| Eco5 ncRNA- | ncRNA-sgRNA fusion (10 bp | GAAAUGAUAAGAUUCCGUACGCCAGCAGUGGCAAUAGCGUUUCC |
| sgRNA | insertion in EMX1 gene) | GGCCUUUUGUGCCGGGAGGGUCGGCGAGUCGCUGACUUAACGCC |
| | | AGUAGUAUGUCCAUAUACCCAAAGUCGCUUCAUUGUAACAAACG |
| | | GCAGAAGCUGGAGGAGGAAGGGCCUGAGUCCGAGCAGAAGAAU |
| | | CCUUCGAGCAAAGUUCUCCCAUCACAUCAACCGGUGGCGCAUUG |
| | | CCACGAAGCAGGCCAAUGGGGAGGACAUCGAUGUCAUACAGUUA |
| | | CGCGCCUUCGGGAUGGUUUAAUGGUAUUGCCGCUGUUGGCGUAC |
| | | GGAAUCUUAUCAGAGUCCGAGCAGAAGAAGAAGUUUCAGAGCU |
| | | AUGCUGGAAACAGCAUAGCAAGUUGAAAUAAGGCUAGUCCGUU |
| | | AUCAACUUGAAAAAGUGGCACCGAGUCGGUGCUUUUUUUUUUU |
| | | U (SEQ ID NO: 19131) |
| Cas9 | All U are N-1- | GGGUCCCGCAGUCGGCGUCCAGCGGCUCUGCUUGUUCGUGUGUG |
| | methylpseudouridine. | UGUCGUUGCAGGCCUUAUUCGGAUCCGCCACCAUGGACAAGAAG |
| | | UACAGCAUCGGACUGGACAUCGGAACAAACAGCGUCGGAUGGGC |
| | | AGUCAUCACAGACGAAUACAAGGUCCCGAGCAAGAAGUUCAAGG |
| | | UCCUGGGAAACACAGACAGACACAGCAUCAA |
| | | GAAGAACCUGAUCGGAGCACUGCUGUUCGACAGCGGAGAAACAG |
| | | CAGAAGCAACAAGACUGAAGAGAAC |
| | | AGCAAGAAGAAGAUACACAAGAAGAAAGAACAGAAUCUGCUAC |
| | | CUGCAGGAAAUCUUCAGCAACGAAAU |
| | | GGCAAAGGUCGACGACAGCUUCUUCCACAGACUGGAAGAAAGCU |
| | | UCCUGGUCGAAGAAGACAAGAAGCA |
| | | CGAAAGACACCCGAUCUUCGGAAACAUCGUCGACGAAGUCGCAU |
| | | ACCACGAAAAGUACCCGACAAUCUAC |
| | | CACCUGAGAAAGAAGCUGGUCGACAGCACAGACAAGGCAGACCU |
| | | GAGACUGAUCUACCUGGCACUGGCA |
| | | CACAUGAUCAAGUUCAGAGGACACUUCCUGAUCGAAGGAGACCU |
| | | GAACCCGGACAACAGCGACGUCGAC |
| | | AAGCUGUUCAUCCAGCUGGUCCAGACAUACAACCAGCUGUUCGA |
| | | AGAAAACCCGAUCAACGCAAGCGGA |
| | | GUCGACGCAAAGGCAAUCCUGAGCGCAAGACUGAGCAAGAGCAG |
| | | AAGACUGGAAAACCUGAUCGCACAG |
| | | CUGCCGGGAGAAAAGAAGAACGGACUGUUCGGAAACCUGAUCGC |
| | | ACUGAGCCUGGGACUGACACCGAAC |
| | | UUCAAGAGCAACUUCGACCUGGCAGAAGACGCAAAGCUGCAGCU |
| | | GAGCAAGGACACAUACGACGACGAC |
| | | CUGGACAACCUGCUGGCACAGAUCGGAGACCAGUACGCAGACCU |
| | | GUUCCUGGCAGCAAAGAACCUGAGC |
| | | GACGCAAUCCUGCUGAGCGACAUCCUGAGAGUCAACACAGAAAU |
| | | CACAAAGGCACCGCUGAGCGCAAGC |
| | | AUGAUCAAGAGAUACGACGAACACCACCAGGACCUGACACUGCU |
| | | GAAGGCACUGGUCAGACAGCAGCUG |
| | | CCGGAAAAGUACAAGGAAAUCUUCUUCGACCAGAGCAAGAACGG |
| | | AUACGCAGGAUACAUCGACGGAGGA |
| | | GCAAGCCAGGAAGAAUUCUACAAGUUCAUCAAGCCGAUCCUGGA |
| | | AAAGAUGGACGGAACAGAAGAACUG |
| | | CUGGUCAAGCUGAACAGAGAAGACCUGCUGAGAAAGCAGAGAA |
| | | CAUUCGACAACGGAAGCAUCCCGCAC |
| | | CAGAUCCACCUGGGAGAACUGCACGCAAUCCUGAGAAGACAGGA |
| | | AGACUUCUACCCGUUCCUGAAGGAC |
| | | AACAGAGAAAAGAUCGAAAAGAUCCUGACAUUCAGAAUCCCGU |
| | | ACUACGUCGGACCGCUGGCAAGAGGA |
| | | AACAGCAGAUUCGCAUGGAUGACAAGAAAGAGCGAAGAAACAA |
| | | UCACACCGUGGAACUUCGAAGAAGUC |
| | | GUCGACAAGGGAGCAAGCGCACAGAGCUUCAUCGAAAGAAUGAC |
| | | AAACUUCGACAAGAACCUGCCGAAC |
| | | GAAAAGGUCCUGCCGAAGCACAGCCUGCUGUACGAAUACUUCAC |
| | | AGUCUACAACGAACUGACAAAGGUC |
| | | AAGUACGUCACAGAAGGAAUGAGAAAGCCGGCAUUCCUGAGCG |
| | | GAGAACAGAAGAAGGCAAUCGUCGAC |
| | | CUGCUGUUCAAGACAAACAGAAAGGUCACAGUCAAGCAGCUGAA |
| | | GGAAGACUACUUCAAGAAGAUCGAA |
| | | UGCUUCGACAGCGUCGAAAUCAGCGGAGUCGAAGACAGAUUCAA |
| | | CGCAAGCCUGGGAACAUACCACGAC |
| | | CUGCUGAAGAUCAUCAAGGACAAGGACUUCCUGGACAACGAAGA |
| | | AAACGAAGACAUCCUGGAAGACAUC |
| | | GUCCUGACACUGACACUGUUCGAAGACAGAGAAAUGAUCGAAG |
| | | AAAGACUGAAGACAUACGCACACCUG |
| | | UUCGACGACAAGGUCAUGAAGCAGCUGAAGAGAAGAAGAUACA |
| | | CAGGAUGGGGAAGACUGAGCAGAAAG |
| | | CUGAUCAACGGAAUCAGAGACAAGCAGAGCGGAAAGACAAUCCU |
| | | GGACUUCCUGAAGAGCGACGGAUUC |
| | | GCAAACAGAAACUUCAUGCAGCUGAUCCACGACGACAGCCUGAC |
| | | AUUCAAGGAAGACAUCCAGAAGGCA |
| | | CAGGUCAGCGGACAGGGAGACAGCCUGCACGAACACAUCGCAAA |
| | | CCUGGCAGGAAGCCCGGCAAUCAAG |
| | | AAGGGAAUCCUGCAGACAGUCAAGGUCGUCGACGAACUGGUCAA |
| | | GGUCAUGGGAAGACACAAGCCGGAA |
| | | AACAUCGUCAUCGAAAUGGCAAGAGAAAACCAGACAACACAGAA |
| | | GGGACAGAAGAACAGCAGAGAAAGAAUGAAGAGAAUCGAAGAA |
| | | GGAAUCAAGGAACUGGGAAGCCAGAUCCUGAAGGAACACCCGGU |
| | | CGAAAAC |
| | | ACACAGCUGCAGAACGAAAAGCUGUACCUGUACUACCUGCAGAA |
| | | CGGAAGAGACAUGUACGUCGACCAG |
| | | GAACUGGACAUCAACAGACUGAGCGACUACGACGUCGACCACAU |
| | | CGUCCCGCAGAGCUUCCUGAAGGACG |
| | | ACAGCAUCGACAACAAGGUCCUGACAAGAAGCGACAAGAACAGA |
| | | GGAAAGAGCGACAACGUCCCGAGCG |
| | | AAGAAGUCGUCAAGAAGAUGAAGAACUACUGGAGACAGCUGCU |
| | | GAACGCAAAGCUGAUCACACAGAGAA |
| | | AGUUCGACAACCUGACAAAGGCAGAGAGAGGAGGACUGAGCGA |
| | | ACUGGACAAGGCAGGAUUCAUCAAGA |
| | | GACAGCUGGUCGAAACAAGACAGAUCACAAAGCACGUCGCACAG |
| | | AUCCUGGACAGCAGAAUGAACACAA |
| | | AGUACGACGAAAACGACAAGCUGAUCAGAGAAGUCAAGGUCAU |
| | | CACACUGAAGAGCAAGCUGGUCAGCG |
| | | ACCUCAGAAAGGACUUCCAGUUCUACAAGGUCAGAGAAAUCAAC |
| | | AACUACCACCACGCACACGACGCAU |
| | | ACCUGAACGCAGUCGUCGGAACAGCACUGAUCAAGAAGUACCCG |
| | | AAGCUGGAAAGCGAAUUCGUCUACG |
| | | GAGACUACAAGGUCUACGACGUCAGAAAGAUGAUCGCAAAGAG |
| | | CGAACAGGAAAUCGGAAAGGCAACAG |
| | | CAAAGUACUUCUUCUACAGCAACAUCAUGAACUUCUUCAAGACA |
| | | GAAAUCACACUGGCAAACGGAGAAA |
| | | UCAGAAAGAGACCGCUGAUCGAAACAAACGGAGAAACAGGAGA |
| | | AAUCGUCUGGGACAAGGGAAGAGACU |
| | | UCGCAACAGUCAGAAAGGUCCUGAGCAUGCCGCAGGUCAACAUC |
| | | GUCAAGAAGACAGAAGUCCAGACAG |
| | | GAGGAUUCAGCAAGGAAAGCAUCCUGCCGAAGAGAAACAGCGAC |
| | | AAGCUGAUCGCAAGAAAGAAGGACU |
| | | GGGACCCGAAGAAGUACGGAGGAUUCGACAGCCCGACAGUCGCA |
| | | UACAGCGUCCUGGUCGUCGCAAAGG |
| | | UCGAAAAGGGAAAGAGCAAGAAGCUGAAGAGCGUCAAGGAACU |
| | | GCUGGGAAUCACAAUCAUGGAAAGAAGCAGCUUCGAAAAGAAC |
| | | CCGAUCGACUUCCUGGAAGCAAAGGGAUACAAGGAAGUCAAGA |
| | | AGGACCUGA |
| | | UCAUCAAGCUGCCGAAGUACAGCCUGUUCGAACUGGAAAACGGA |
| | | AGAAAGAGAAUGCUGGCAAGCGCAG |
| | | GAGAACUGCAGAAGGGAAACGAACUGGCACUGCCGAGCAAGUAC |
| | | GUCAACUUCCUGUACCUGGCAAGCC |
| | | ACUACGAAAAGCUGAAGGGAAGCCCGGAAGACAACGAACAGAA |
| | | GCAGCUGUUCGUCGAACAGCACAAGC |
| | | ACUACCUGGACGAAAUCAUCGAACAGAUCAGCGAAUUCAGCAAG |
| | | AGAGUCAUCCUGGCAGACGCAAACC |
| | | UGGACAAGGUCCUGAGCGCAUACAACAAGCACAGAGACAAGCCG |
| | | AUCAGAGAACAGGCAGAAAACAUCA |
| | | UCCACCUGUUCACACUGACAAACCUGGGAGCACCGGCAGCAUUC |
| | | AAGUACUUCGACACAACAAUCGACAG |
| | | AAAGAGAUACACAAGCACAAAGGAAGUCCUGGACGCAACACUGA |
| | | UCCACCAGAGCAUCACAGGACUGUA |
| | | CGAAACAAGAAUCGACCUGAGCCAGCUGGGAGGAGACGGAGGA |
| | | GGAAGCCCGAAGAAGAAGAGAAAGGU |
| | | CUAGCUAGCCAUCACAUUUAAAAGCAUCUCAGCCUACCAUGAGA |
| | | AUAAGAGAAAGAAAAUGAAGAUCAA |
| | | UAGCUUAUUCAUCUCUUUUUCUUUUUCGUUGGUGUAAAGCCAA |
| | | CACCCUGUCUAAAAAACAUAAAUUUC |
| | | UUUAAUCAUUUUGCCUCUUUUCUCUGUGCUUCAAUUAAUAAAA |
| | | AAUGGAAAGAACCUCGAGAAAAAAAA |
| | | AAAAAAAAAAAAAAAAAAAAAAAAAAAAAAAAAAAAAAAAAAA |
| | | AAAAAAAAAAAAAAAAAAAAAAAAA |
| | | AAAAAAAAAAAAAAUCUAG (SEQ ID NO: 19132) |
| EMX1 | Contains chemical | GAGUCCGAGCAGAAGAAGAAGUUUCAGAGCUAUGCUGGAAACA |
| sgRNA | modifications from Synthego: | GCAUAGCAAGUUGAAAUAAGGCUAGUCCGUUAUCAACUUGAAA |
| modified | 2′-F, 2′-O-methyl, | AAGUGGCACCGAGUCGGUGCUUUUU (SEQ ID NO: 19133) |
| | phosphorothioates | |
| EMX1 | No modifications | GAGUCCGAGCAGAAGAAGAAGUUUCAGAGCUAUGCUGGAAACA |
| sgRNA | | GCAUAGCAAGUUGAAAUAAGGCUAGUCCGUUAUCAACUUGAAA |
| unmodified | | AAGUGGCACCGAGUCGGUGCUUUUU (SEQ ID NO: 19134) |
| Eco3 ncRNA | Eco3 ncRNA only with 10 bp | GGGAUAAUUGAUAAGAUUCCGUAAGAGCCAAACCUAGCAUUUU |
| | insertion | AUGGGUUAAUAGCCCAUCGGGCCAUGAGUCAUGGUUUCGCCUAG |
| | | UAUUUUAGCUAUGCCCGUCGUUCAGUUCGCUGAACAAACGGCAG |
| | | AAGCUGGAGGAGGAAGGGCCUGAGUCCGAGCAGAAGAAUUACG |
| | | UCUGCAAAGUUCUCCCAUCACAUCAACCGGUGGCGCAUUGCCAC |
| | | GAAGCAGGCCAAUGGGGAGGACAUCGAUGUCAUCAGCGAACUG |
| | | AUCGACGUGCUCAAGUAGGUUUGGCUCUUACGGAAUCUUAUCA |
| | | GAU (SEQ ID NO: 19135) |
| Eco3 ncRNA- | Eco3 ncRNA only with 10 bp | GGGAUAAUUGAUAAGAUUCCGUAAGAGCCAAACCUAGCAUUUU |
| MS2 | insertion and 3′ MS2 stem loop | AUGGGUUAAUAGCCCAUCGGGCCAUGAGUCAUGGUUUCGCCUAG |
| | | UAUUUUAGCUAUGCCCGUCGUUCAGUUCGCUGAACAAACGGCAG |
| | | AAGCUGGAGGAGGAAGGGCCUGAGUCCGAGCAGAAGAAUUACG |
| | | UCUGCAAAGUUCUCCCAUCACAUCAACCGGUGGCGCAUUGCCAC |
| | | GAAGCAGGCCAAUGGGGAGGACAUCGAUGUCAUCAGCGAACUG |
| | | AUCGACGUGCUCAAGUAGGUUUGGCUCUUACGGAAUCUUAUCA |
| | | ACAUGAGGAUCACCCAUGUGAU (SEQ ID NO: 19136) |
| Aco1 RT | Aco1 RT RNA sequence | AGGGGGAAAUAAGAGAGAAAAGAAGAGUAAGAAGAAAUAUAAG |
| | including 5′ and 3′ UTRs | AGCCACCAUGGAGCCCAAUGACUACGUAAAUAGGUUGCGACAUG |
| | | CUAUGGAAAUAAGUGAAAACCCGCGCUUUAGCCCUGAGUACAUU |
| | | GCUCAAUGUUGUACUUACGCCGAGAAUCUCCUCAAGCAAGGGCU |
| | | GCCAGUUCUGUUCGAUCAAACGCAUAUUCGGAAAGUUCUGGGG |
| | | AUGGCGGCACCUCGAUUGUGUGAUUAUCACAGAUUCACAAUCCC |
| | | AAAGCACAACGGAUCUAGAAUUAUCACGGCCCCAUCUAGGAAGC |
| | | UGAAGCUUCGACAACAAUGGAUAUACCAGAAUAUCCUUAUACG |
| | | AAAGGAGGCUUCACCGUACACGCACGGAUUUGUUCCUGAACGCA |
| | | GCAUCGUGACUAACGCAAUCCUCCAUAUAGGAUACGCAUACACC |
| | | UACUGCGUGGAUAUCACGGAUUUCUUUCCUAGCAUCACUAAGAA |
| | | GCAGGUCUUGCCUAUAUUCCGAAAUAUGGGCUAUAGUGGUUCU |
| | | GCUGCAAAUACUCUCUGCGACCUCUGUUGCUAUGACGGGGUCCU |
| | | CCCCCAGGGGGCGCCUACUAGCCCAUACCUCAGUAACAUGAUUU |
| | | GUCGCGAUCUUGAUGACGAAUUGGGGGCUAUGGCGCGGCGGUU |
| | | CCGGGGGAUUUUUACACGGUAUGCGGAUGACAUAGCUAUCUCCA |
| | | CAAACCAACAACAGCCGCAACUUUUGGAUGCCUUGGGACUUAUC |
| | | CUCGGGAAGCACGGAUUUCUUAUGAAUCUCGAUAAGUGUCGAG |
| | | UCUAUAAUCCUGGACAGCCCAAAAGAAUUACUGGAUUGACCGUU |
| | | CACAAUAGAGUAUCAGUUCCGAAAACCUUUAAGCGGAAAUUGC |
| | | GGCAGGAAAUACAUUACUGUCAGAAGUUCGGAGUGACUGCACA |
| | | UUUGGAAAACACGAAGGCUGCACGAUCCAUCCACUAUAGGGAAC |
| | | AUCUGUAUGGAAAGGCAUACUAUGUUAAAAUGGUUGAGCCUGA |
| | | GCUCGGGGCGCACUUCCUCGAUGAGUUGUCAAAGGUAGACUGGC |
| | | CAGAGCCACCAAAGAAGAAAAGAAAGGUCUGAUAAUAGGCUGG |
| | | AGCCUCGGUGGCCAUGCUUCUUGCCCCUUGGGCCUCCCCCCAGC |
| | | CCCUCCUCCCCUUCCUGCACCCGUACCCCCGUGGUCUUUGAAUA |
| | | AAGUCUGAGUGGGCGGCCCUGCA (SEQ ID NO: 19137) |
| Aco1 | Aco1 ncRNA-sgRNA with 50 | GGGGUAUAAAACCGGGAACGAUCAGACCGGGGUGAAUUCGCCCC |
| ncRNA- | bp insertion | CUUGAUCAAACGGCACUAACCACUGUUUGCCGUGCGUGCGUACA |
| sgRNA_50 | | AACGGCAGAAGCUGGAGGAGGAAGGGCCUGAGUCCGAGCAGAA |
| | | GAAAACUCGAGCUCUGAUCAGUAUCAUGGGGCCCAUGGUUGAA |
| | | UGACUCCUAUAAAGUUCUCCCAUCACAUCAACCGGUGGCGCAUU |
| | | GCCACGAAGCAGGCCAAUGGGGAGGACAUCGAUGUCAACGCACG |
| | | CACGGCAAACAGACAGAUCCAUUACUAUUACAAUUUAUUUAGU |
| | | GAUCGUUCCCGGUUUUAUACGAGUCCGAGCAGAAGAAGAAGUU |
| | | UCAGAGCUAUGCUGGAAACAGCAUAGCAAGUUGAAAUAAGGCU |
| | | AGUCCGUUAUCAACUUGAAAAAGUGGCACCGAGUCGGUGCUUU |
| | | UUCCUGCA (SEQ ID NO: 19138) |
| Aco1 sgRNA- | Aco1 sgRNA-ncRNA with 50 | GGGGAGUCCGAGCAGAAGAAGAAGUUUCAGAGCUAUGCUGGAA |
| ncRNA_50 | bp insertion | ACAGCAUAGCAAGUUGAAAUAAGGCUAGUCCGUUAUCAACUUG |
| | | AAAAAGUGGCACCGAGUCGGUGCUUUUUGUAUAAAACCGGGAA |
| | | CGAUCAGACCGGGGUGAAUUCGCCCCCUUGAUCAAACGGCACUA |
| | | ACCACUGUUUGCCGUGCGUGCGUACAAACGGCAGAAGCUGGAGG |
| | | AGGAAGGGCCUGAGUCCGAGCAGAAGAAAACUCGAGCUCUGAUC |
| | | AGUAUCAUGGGGCCCAUGGUUGAAUGACUCCUAUAAAGUUCUCC |
| | | CAUCACAUCAACCGGUGGCGCAUUGCCACGAAGCAGGCCAAUGG |
| | | GGAGGACAUCGAUGUCAACGCACGCACGGCAAACAGACAGAUCC |
| | | AUUAUUAUUACAAUUUAUUUAGUGAUCGUUCCCGGUUUUAUAC |
| | | CCUGCA (SEQ ID NO: 19139) |
| Eco3_EMX1 | Eco3 ncRNA containing 25 bp | GAGUCCGAGCAGAAGAAGAAGUUUCAGAGCUAUGCUGGAAACA |
| gRNA_ | insertion in EMX1 gene fused | GCAUAGCAAGUUGAAAUAAGGCUAGUCCGUUAUCAACUUGAAA |
| ncRNA_25 | to EMX1 sgRNA at 5′ end | AAGUGGCACCGAGUCGGUGCUUUUUAAUAACAACGAAAUGAUA |
| | | AGAUUCCGUAAGAGCCAAACCUAGCAUUUUAUGGGUUAAUAGC |
| | | CCAUCGGGCCAUGAGUCAUGGUUUCGCCUAGUAUUUUAGCUAUG |
| | | CCCGUCGUUCAGUUCGCUGAACAAACGGCAGAAGCUGGAGGAGG |
| | | AAGGGCCUGAGUCCGAGCAGAAGAAaACUCGAGCUCUGAAUGAC |
| | | UCCUAUAaaguuCUCCCAUCACAUCAACCGGUGGCGCAUUGCCACG |
| | | AAGCAGGCCAAUGGGGAGGACAUCGAUGUCAUCAGCGAACUGA |
| | | UCGACGUGCUCAAGUAGGUUUGGCUCUUACGGAAUCUUAUCA |
| | | (SEQ ID NO: 19140) |
| Eco3_ncRNA | Eco3 ncRNA containing 25 bp | GAAAUGAUAAGAUUCCGUAAGAGCCAAACCUAGCAUUUUAUGG |
| only | insertion in EMX1 gene | GUUAAUAGCCCAUCGGGCCAUGAGUCAUGGUUUCGCCUAGUAUU |
| | | UUAGCUAUGCCCGUCGUUCAGUUCGCUGAACAAACGGCAGAAGC |
| | | UGGAGGAGGAAGGGCCUGAGUCCGAGCAGAAGAAUUACGUCUG |
| | | CAAAGUUCUCCCAUCACAUCAACCGGUGGCGCAUUGCCACGAAG |
| | | CAGGCCAAUGGGGAGGACAUCGAUGUCAUCAGCGAACUGAUCGA |
| | | CGUGCUCAAGUAGGUUUGGCUCUUACGGAAUCUUAUCA(SEQ ID |
| | | NO: 19141) |
| Eco3_ncRNA_ | Eco3 ncRNA containing 25 bp | GAAAUGAUAAGAUUCCGUAAGAGCCAAACCUAGCAUUUUAUGG |
| AAVSI | insertion in AAVSI gene fused | GUUAAUAGCCCAUCGGGCCAUGAGUCAUGGUUUCGCCUAGUAUU |
| gRNA_25 | to AAVSI sgRNA at 3′ end | UUAGCUAUGCCCGUCGUUCAGUUCGCUGACUGUCCCCUCCACCC |
| | | CACAGUGGGGCCACUAGGGACAGAAAUGACAGUGGUUGGUGCU |
| | | CUAAAAAUUAAUGACAGAAAAGCCCCAUCCUUAGGCCUCCUCCU |
| | | UCCUAGUCUCCUGAUAUUGGGUCUAACCCCCAUCAGCGAACUGA |
| | | UCGACGUGCUCAAGUAGGUUUGGCUCUUACGGAAUCUUAUCAA |
| | | AUAACAACGGGGCCACUAGGGACAGGAUGUUUCAGAGCUAUGC |
| | | UGGAAACAGCAUAGCAAGUUGAAAUAAGGCUAGUCCGUUAUCA |
| | | ACCUGAAAAAGUGGCACCGAGUCGGUGCUUUUU(SEQ ID NO: |
| | | 19142) |
| Eco3_ncRNA_ | Eco3 ncRNA containing 25 bp | GAAAUGAUAAGAUUCCGUAAGAGCCAAACCUAGCAUUUUAUGG |
| EMX1 | insertion in EMX1 gene fused | GUUAAUAGCCCAUCGGGCCAUGAGUCAUGGUUUCGCCUAGUAUU |
| gRNA_25 | to EMX1 sgRNA at 3′ end | UUAGCUAUGCCCGUCGUUCAGUUCGCUGAACAAACGGCAGAAGC |
| | | UGGAGGAGGAAGGGCCUGAGUCCGAGCAGAAGAAaACUCGAGCU |
| | | CUGAAUGACUCCUAUAaaguuCUCCCAUCACAUCAACCGGUGGCGC |
| | | AUUGCCACGAAGCAGGCCAAUGGGGAGGACAUCGAUGUCAUCAG |
| | | CGAACUGAUCGACGUGCUCAAGUAGGUUUGGCUCUUACGGAAUC |
| | | UUAUCAAAUAACAACGAGUCCGAGCAGAAGAAGAAGUUUCAGA |
| | | GCUAUGCUGGAAACAGCAUAGCAAGUUGAAAUAAGGCUAGUCC |
| | | GUUAUCAACUUGAAAAAGUGGCACCGAGUCGGUGCUUUUU(SEQ |
| | | ID NO: 19143) |
| Eco3_ncRNA_ | Eco3 ncRNA containing 50 bp | GAAAUGAUAAGAUUCCGUAAGAGCCAAACCUAGCAUUUUAUGG |
| EMX1 | insertion in EMX1 gene fused | GUUAAUAGCCCAUCGGGCCAUGAGUCAUGGUUUCGCCUAGUAUU |
| gRNA_50 | to EMX1 sgRNA at 3′ end | UUAGCUAUGCCCGUCGUUCAGUUCGCUGAACAAACGGCAGAAGC |
| | | UGGAGGAGGAAGGGCCUGAGUCCGAGCAGAAGAAaACUCGAGCU |
| | | CUGAUCAGUAUCAUGGGGCCCAUGGUUGAAUGACUCCUAUAaagu |
| | | uCUCCCAUCACAUCAACCGGUGGCGCAUUGCCACGAAGCAGGCC |
| | | AAUGGGGAGGACAUCGAUGUCAUCAGCGAACUGAUCGACGUGC |
| | | UCAAGUAGGUUUGGCUCUUACGGAAUCUUAUCAAAUAACAACG |
| | | AGUCCGAGCAGAAGAAGAAGUUUCAGAGCUAUGCUGGAAACAG |
| | | CAUAGCAAGUUGAAAUAAGGCUAGUCCGUUAUCAACUUGAAAA |
| | | AGUGGCACCGAGUCGGUGCUUUUU(SEQ ID NO: 19144) |
| Eco3_ncRNA_ | Eco3 ncRNA containing 75 bp | GAAAUGAUAAGAUUCCGUAAGAGCCAAACCUAGCAUUUUAUGG |
| EMX1 | insertion in EMX1 gene fused | GUUAAUAGCCCAUCGGGCCAUGAGUCAUGGUUUCGCCUAGUAUU |
| gRNA_75 | to EMX1 sgRNA at 3′ end | UUAGCUAUGCCCGUCGUUCAGUUCGCUGAACAAACGGCAGAAGC |
| | | UGGAGGAGGAAGGGCCUGAGUCCGAGCAGAAGAAaACUCGAGCU |
| | | CUGAGCCCCACCGCAGCUUGCCAGCACUUUCAGUAUCAUGGGGC |
| | | CCAUGGUUGAAUGACUCCUAUAaaguuCUCCCAUCACAUCAACCGG |
| | | UGGCGCAUUGCCACGAAGCAGGCCAAUGGGGAGGACAUCGAUGU |
| | | CAUCAGCGAACUGAUCGACGUGCUCAAGUAGGUUUGGCUCUUAC |
| | | GGAAUCUUAUCAAAUAACAACGAGUCCGAGCAGAAGAAGAAGU |
| | | UUCAGAGCUAUGCUGGAAACAGCAUAGCAAGUUGAAAUAAGGC |
| | | UAGUCCGUUAUCAACUUGAAAAAGUGGCACCGAGUCGGUGCUU |
| | | UUU(SEQ ID NO: 19145) |
| Eco3_ncRNA_ | Eco3 ncRNA containing 100 | GAAAUGAUAAGAUUCCGUAAGAGCCAAACCUAGCAUUUUAUGG |
| EMX1 | bp insertion in EMX1 gene | GUUAAUAGCCCAUCGGGCCAUGAGUCAUGGUUUCGCCUAGUAUU |
| gRNA_100 | fused to EMX1 sgRNA at 3′ | UUAGCUAUGCCCGUCGUUCAGUUCGCUGAACAAACGGCAGAAGC |
| | end | UGGAGGAGGAAGGGCCUGAGUCCGAGCAGAAGAAaACUCGAGCU |
| | | CUGAGCCCCACUGUCGAGAAGUAUGUAUCUCGCUCCCGCAGCUU |
| | | GCCAGCACUUUCAGUAUCAUGGGGCCCAUGGUUGAAUGACUCCU |
| | | AUAaaguuCUCCCAUCACAUCAACCGGUGGCGCAUUGCCACGAAGC |
| | | AGGCCAAUGGGGAGGACAUCGAUGUCAUCAGCGAACUGAUCGAC |
| | | GUGCUCAAGUAGGUUUGGCUCUUACGGAAUCUCAUCAAAUAAC |
| | | AACGAGUCCGAGCAGAAGAAGAAGUUUCAGAGCUAUGCUGGAA |
| | | ACAGCAUAGCAAGUUGAAAUAAGGCUAGUCCGUUAUCAACUUG |
| | | AAAAAGUGGCACCGAGUCGGUGCUUUUU(SEQ ID NO: 19146) |
| Eco3_ncRNA_ | Eco3 ncRNA containing GFP | GAAAUGAUAAGAUUCCGUAAGAGCCAAACCUAGCAUUUUAUGG |
| EMX1 | gene insertion in EMX1 gene | GUUAAUAGCCCAUCGGGCCAUGAGUCAUGGUUUCGCCUAGUAUU |
| gRNA_GFP | fused to EMX1 sgRNA at 3′ | UUAGCUAUGCCCGUCGUUCAGUUCGCUGAACAAACGGCAGAAGC |
| gene | end. Entire GFP cassette is in | UGGAGGAGGAAGGGCCUGAGUCCGAGCAGAAGAAcccauauguccuuc |
| | antisense orientation and | cgagugagagacacaaaaaauuccaacacacuauugcaaugaaaauaa |
| | contains mini EFla promoter | auuuccuuuuuagccagaagucagaugcucaaggggcuucaugauguc |
| | and beta globin polyA signal. | cccauaauuuuuggcagagggaaaaagaucucagugguauuugugagc |
| | | cagggcauuggccacaccagccaccaccuucugauaggcagccugcac |
| | | cugaggagugcggccgcuuuacuuguacagcucguccaugccgagagu |
| | | gaucccggggggucacgaacucagugguugucgggcagcagcacgggg |
| | | ccgucgccgauggggguguucugcugguaguggucggcgcuugucggc |
| | | caugauauagacguuguggcuguuuuuuuuuugagauucaccucgggg |
| | | ggucuuguaguugccgucguccuugaagaagaugguggcuccuggacg |
| | | uagccuucgggcauggcggacuugaagaagucgugcugcuucaugugg |
| | | ucgggguagcggcugaagcacugcacgccguaggucaggguggucacg |
| | | agggugggccagggcacgggcagcuugccgguggugcagaugaacuuc |
| | | agggucaguugccguagguggcaucgcccucgcccucgccggacacgu |
| | | gaacuuguggccguuuacgucgccguccagcucgaccaggaugggcac |
| | | caccccggugaacagcuccucgcccuugcucaccaugguggcgaccgg |
| | | uggaucccgggcccgcgguaccgucgacugcagaauucgaagcuugag |
| | | cucgagaucugaguccgguagcgcuagggaucugacgguucacuaaac |
| | | ccuguguuccacccucgggaaaaaggcggagccaguacacgacaucac |
| | | uuucccaguuuaccccgcgccaccuucucuaggcaccgguucaauugc |
| | | cgaccccuccccccaacuucucggggacuguggggaugugcgcu |
| | | cugcccguuCUCCCAUCACAUCAACCGGUGGCGCAUUGCCACGAAGC |
| | | AGGCCAAUGGGGAGGACAUCGAUGUCAUCAGCGAACUGAUCGAC |
| | | GUGCUCAAGUAGGUUUGGCUCUUACGGAAUCUUAUCAAAUAAC |
| | | AACGAGUCCGAGCAGAAGAAGAAGUUUCAGAGCUAUGCUGGAA |
| | | ACAGCAUAGCAAGUUGAAAUAAGGCUAGUCCGUUAUCAACUUG |
| | | AAAAAGUGGCACCGAGUCGGUGCUUUUU(SEQ ID NO: 19147) |
| Eco3_NT_nc | Eco3 ncRNA containing 25 bp | GAAAUGAUAAGAUUCCGUAAGAGCCAAACCUAGCAUUUUAUGG |
| RNA_EMX | insertion (on opposite strand of | GUUAAUAGCCCAUCGGGCCAUGAGUCAUGGUUUCGCCUAGUAUU |
| gRNA_25 | sgRNA cut) in EMX1 gene | UUAGCUAUGCCCGUCGUUCAGUUCGCUGAugacaucgauguccucccca |
| | fused to EMX1 sgRNA at 3′ | uuggccugcuucguggcaaugcgccaccgguugaugugaugggagaacu |
| | end | uUAUAGGAGUCAuuuguUCAGCGAACUGAUCGACGUGCUCAAGUAGGUU |
| | | UGGCUCUUACGGAAUCUUAUCAAAUAACAACGAGUCCGAGCAGAAGAAG |
| | | AAGUUUCAGAGCUAUGCUGGAAACAGCAUAGCAAGUUGAAAUAAG |
| | | GCUAGUCCGUUAUCAACUUGAAAAAGUGGCACCGAGUCGGUGCU |
| | | UUUU(SEQ ID NO: 19148) |
| Aco1_EMX1 | Aco1 ncRNA containing 25 bp | gaguccgagcagaagaagaaguuucagagcuaugcuggaaacagcauag |
| gRNA_ncRN | insertion in EMX1 gene fused | caaguugaaauaaggcuaguccguuaucaacuugaaaaaguggcaccga |
| A 25 | to EMX1 sgRNA at 5′ end | gucggugcuuuuuAAUAACAACguauaaaaccgggaacgaucagaccgg |
| | | ggugaauucgcccccuugaucaaacggcacuaaccacuguuugccgugc |
| | | gugcguacaaacggcagaagcuggaggaggaagggccugaguccgagca |
| | | gaagaaaACUCGAGCUCUGAAUGACUCCUAUAaaguucucccaucacau |
| | | caaccgguggcgcauugccacgaagcaggccaauggggaggacaucgau |
| | | gucaacgcacgcacggcaaacagacagauccauuauuuuuuuuuuu |
| | | (SEQ ID NO: 19149) |
| Aco1_ncRNA | Aco1 ncRNA containing 25 bp | guauaaaaccgggaacgaucagaccggggugaauucgcccccuugaucaa |
| only | insertion in EMX1 gene | acggcacuaaccacuguuugccgugcgugcguacaaacggcagaagcugg |
| | | aggaggaagggccugaguccgagcagaagaaaACUCGAGCUCUGAAUGAC |
| | | UCCUAUAaaguucucccaucacaucaaccgguggcgcauugccacgaagc |
| | | aggccaauggggaggacaucgaugucaacgcacgcacggcaaacagacag |
| | | auccauuauuauuacaauuuauuuagugaucguucccgguuuuauac |
| | | (SEQ ID NO: 19150) |
| Aco1_ncRNA_ | Aco1 ncRNA containing 25 bp | guauaaaaccgggaacgaucagaccggggugaauucgcccccuugaucaaa |
| AAVS1 | insertion in AAVSI gene fused | cggcacuaaccacuguuugccgugcgugcguUCUGUCCCCUCCACCCCACA |
| gRNA_25 | to AAVSI sgRNA at 3′ end | GUGGGGCCACUAGGGACAGAAAUGACAGUGGUUGGUGCUCUAAAAAUUAAU |
| | | GACAGAAAAGCCCCAUCCUUAGGCCUCCUCCUUCCUAGUCUCCUGAUAUUG |
| | | GGUCUAACCCCCAacgcacgcacggcaaacagacagauccauuauuauuac |
| | | aauuuauuuagugaucguucccgguuuuauacAAUAACAACGGGGCCACUA |
| | | GGGACAGGAUguuucagagcuaugcuggaaacagcauagcaaguugaaaua |
| | | aggcuaguccguuaucaacuugaaaaaguggcaccgagucggugcuuuuu |
| | | (SEQ ID NO: 19151) |
| Aco1_ncRNA_ | Aco1 ncRNA containing 25 bp | guauaaaaccgggaacgaucagaccggggugaauucgcccccuugaucaa |
| EMX1 | insertion in EMX1 gene fused | acggcacuaaccacuguuugccgugcgugcguacaaacggcagaagcugg |
| gRNA_25 | to EMX1 sgRNA at 3′ end | aggaggaagggccugaguccgagcagaagaaaACUCGAGCUCUGAAUGAC |
| | | UCCUAUAaaguucucccaucacaucaaccgguggcgcauugccacgaagc |
| | | aggccaauggggaggacaucgaugucaacgcacgcacggcaaacagacag |
| | | aucgcagaagaagaaguuucagagcuaugcuggaaacagcauagcaaguu |
| | | gaaauaaggcuaguccguuaucaacuugaaaaaguggcaccgagucggug |
| | | cuuuuu(SEQ ID NO: 19152) |
| Aco1_ncRNA_ | Aco1 ncRNA containing 50 bp | guauaaaaccgggaacgaucagaccggggugaauucgcccccuugaucaa |
| EMX1 | insertion in EMX1 gene fused | acggcacuaaccacuguuugccgugcgugcguacaaacggcagaagcugg |
| gRNA_50 | to EMX1 sgRNA at 3′ end | aggaggaagggccugaguccgagcagaagaaaACUCGAGCUCUGAAUGAC |
| | | UCCUAUAaaguucucccaucacaucaaccgguggcgcauugccacgaagc |
| | | aggccaauggggaggacaucgaugucaacgcacgcacggcaaacagacag |
| | | aucgcagaagaagaaguuucagagcuaugcuggaaacagcauagcaaguu |
| | | gaaauaaggcuaguccguuaucaacuugaaaaaguggcaccgagucggug |
| | | cuuuuu(SEQ ID NO: 19153) |
| Aco1_ncRNA_ | Aco1 ncRNA containing 75 bp | guauaaaaccgggaacgaucagaccggggugaauucgcccccuugaucaa |
| EMX1 | insertion in EMX1 gene fused | acggcacuaaccacuguuugccgugcgugcguacaaacggcagaagcugg |
| gRNA_75 | to EMX1 sgRNA at 3′ end | aggaggaagggccugaguccgagcagaagaaaACUCGAGCUCUGAGCCCC |
| | | ACCGCAGCUUGCCAGCACUUUCAGUAUCAUGGGGCCCAUGGUUGAAUGAC |
| | | UCCUAUAaaguucucccaucacaucaaccgguggcgcauugccacgaagc |
| | | aggccaauggggaggacaucgaugucaacgcacgcacggcaaaACgaguc |
| | | cgagcagaagaagaaguuucagagcuaugcuggaaacagcauagcaaguu |
| | | gaaauaaggcuaguccguuaucaacuugaaaaaguggcaccgagucggug |
| | | cuuuuu(SEQ ID NO: 19154) |
| Aco1_ncRNA_ | Aco1 ncRNA containing 100 | guauaaaaccgggaacgaucagaccggggugaauucgcccccuugaucaa |
| EMX1 | bp insertion in EMX1 gene | acggcacuaaccacuguuugccgugcgugcguacaaacggcagaagcugg |
| gRNA_100 | fused to EMX1 sgRNA at 3′ | aggaggaagggccugaguccgagcagaagaaa |
| | end | ACUCGAGCUCUGAGCCCCACUGUCGAGAAGUAUGUAUCUCGCUC |
| | | CCGCAGCUUGCCAGCACUUUCAGUAUCAUGGGGCCCAUGGUUGA |
| | | AUGACUCCUAUAaaguucucccaucacaucaaccgguggcgcauugccac |
| | | gaagcaggccaauggggaggacaucgaugucaacgcacgcacggcaaaca |
| | | gacagauccauuauuauuacaauuuagagcuaugcuggaaacagcauagc |
| | | aaguugaaauaaggcuaguccguuaucaacuugaaaaaguggcaccgagu |
| | | cggugcuuuuu(SEQ ID NO: 19155) |
| Aco1_ncRNA_ | Aco1 ncRNA containing GFP | guauaaaaccgggaacgaucagaccggggugaauucgcccccuugaucaa |
| EMX1 | gene insertion in EMX1 gene | acggcacuaaccacuguuugccgugcgugcguacaaacggcagaagcugg |
| gRNA_GFP | fused to EMX1 sgRNA at 3′ | aggaggaagggccugaguccgagcagaagaacccauauguccuuccgagu |
| gene | end. Entire GFP cassette is in | gagagacacaaaaaauuccaacacacuauugcaaugaaaauaaauuuccu |
| | antisense orientation and | uuauuagccagaagucagaugcucaaggggcuucaugauguccccauaau |
| | contains mini EFla promoter | uuuuggcagagggaaaaagaucucagugguauuugugagccagggcauug |
| | and beta globin polyA signal. | gccacaccagccaccaccuucugauaggcagccugcaccugaggagugcg |
| | | gccgcuuuacuuguacagcucguccaugccgagagugaucccggcggcgg |
| | | ucacgaacuccagcaggaccaugugaucgcgcuucucguuggggucuuug |
| | | cucagggggacugggugcucagguagugguugucgggcagcagcacgggg |
| | | ccgucgccgauggggguguucugcugguaguggucggcgagcugcacgcu |
| | | gccguccucgauguuguggcggaucuugaaguucaccuugaugccguucu |
| | | ucugcuugucggccaugauauagacguuguggcuguuguaguuguacucc |
| | | agcuugugccccaggauguugccguccuccuugaagucgaugcccuucag |
| | | cucgaugcgguucaccagggugucgcccucgaacuucaccucgggggguc |
| | | uuguaguugccgucuccuugaagaagauggugcgcuccuggacguagccu |
| | | ucgggcauggggacuugaagaagucgugcugcuucauguggucgggguag |
| | | cggcugaagcacugcacgccguaggucaggguggucacgagggugggcca |
| | | gggcacgggcagcuugccgguggugcagaugaacuucagggucaguugcc |
| | | guagguggaugucugccggacacgcugaacuuguggccguuuacgucgcc |
| | | guccagcucgaccaggaugggcaccaccccggugaacagcuccucgcccu |
| | | ugcucaccaugguggcgaccgguggaucccgggcccgcgguaccgucgac |
| | | ugcagaauucgaagcuugagcucgagaucugaguccgguagcgcuagcgg |
| | | aucugacgguucacuaaacccuguguucuggggcaaacccguugcgaaaa |
| | | agaacguucacggcgacuacugcacuuauauacgguucucccccacccuc |
| | | gggaaaaaggcggagccaguacacgacaucacuuucccaguuuaccccgc |
| | | gccaccuucucuaggcaccgguucaauugccgaccccuccccccaacuuc |
| | | ucggggacugugggcgaugugcgcucugcccguucucccaucacaucaac |
| | | cgguggcgcauugccacgaagcaggccaauggggaggacaucgaugucaa |
| | | cgcacgcacggcaaacagacagauccauuauuauuacaauuuauuuagug |
| | | aucguucccgguuuuauacAAUAACAACgaguccgagcagaagaagaagu |
| | | uucagagcuaugcuggaaacagcauagcaaguugaaauaaggcuaguccg |
| | | uuaucaacuugaaaaaguggcaccgagucggugcuuuuu(SEQ ID NO: |
| | | 19156) |
| Aco1_NT_nc | Aco1 ncRNA containing 25 bp | guauaaaaccgggaacgaucagaccggggugaauucgcccccuugaucaa |
| RNA_EMX1 | insertion (on opposite strand of | acggcacuaaccacuguuugccgugcgugcguugacaucgauguccuccc |
| gRNA_25 | sgRNA cut) in EMX1 gene | cauuggccugcuucguggcaaugcgccaccgguugaugugaugggagaac |
| | fused to EMX1 sgRNA at 3′ | uuUAUAGGAGUCAUUCAGAGCUCGAGUuuucuucugcucggacucaggcc |
| | end | cuuccuccuccagcuucugccguuuguacgcacgcacggcaaacagacag |
| | | auccauuauuauuacaauuuauuuagugaucguucccgguuuuauacAAU |
| | | AACAACgaguccgagcagaagaagaaguuucagagcuaugcuggaaacag |
| | | cauagcaaguugaaauaaggcuaguccguuaucaacuugaaaaaguggca |
| | | ccgagucggugcuuuuu(SEQ ID NO: 19157) |
| R2042_EMX1 | R2042 ncRNA containing 25 | gaguccgagcagaagaagaaguuucagagcuaugcuggaaacag |
| gRNA_ncRN | bp insertion in EMX1 gene | cauagcaaguugaaauaaggcuaguccguuaucaacuugaaaaa |
| A_25 | fused to EMX1 sgRNA at 5′ | guggcaccgagucggugcuuuuuAAUAACAACGCGU |
| | end | UAAGGUGGUUAUAUUCUAGUAUUUAUGAAGUGUAGUCGCUUCG |
| | | AUCGUUAAGGCUGAUUUUAACCUCUGCAUAAUAAUAUCGGUAG |
| | | AUAUUAUUAUGCACGCUCCCUUUAGCAGAGCUAAGAAUCGCUCA |
| | | CUCAGGCACAAGCUUUGAGGACAAACGGCAGAAGCUGGAGGAG |
| | | GAAGGGCCUGAGUCCGAGCAGAAGAAaACUCGAGCUCUGAAUGA |
| | | CUCCUAUAAAGUUCUCCCAUCACAUCAACCGGUGGCGCAUUGCC |
| | | ACGAAGCAGGCCAAUGGGGAGGACAUCGAUGUCACCUCAAAGCU |
| | | UGUGCCUGAGUGAGAGCUAAAGAAAAGAAAAGUAGAAUAAGCC |
| | | ACCUUAACGC(SEQ ID NO: 19158) |
| R2042_ncRN | R2042 ncRNA containing 25 | GCGUUAAGGUGGUUAUAUUCUAGUAUUUAUGAAGUGUAGUCGC |
| A only_25 | bp insertion in EMX1 gene | UUCGAUCGUUAAGGCUGAUUUUAACCUCUGCAUAAUAAUAUCG |
| | | GUAGAUAUUAUUAUGCACGCUCCCUUUAGCAGAGCUAAGAAUC |
| | | GCUCACUCAGGCACAAGCUUUGAGGACAAACGGCAGAAGCUGGA |
| | | GGAGGAAGGGCCUGAGUCCGAGCAGAAGAAaACUCGAGCUCUGA |
| | | AUGACUCCUAUAAAGUUCUCCCAUCACAUCAACCGGUGGCGCAU |
| | | UGCCACGAAGCAGGCCAAUGGGGAGGACAUCGAUGUCACCUCAA |
| | | AGCUUGUGCCUGAGUGAGAGCUAAAGAAAAGAAAAGUAGAAUA |
| | | AGCCACCUUAACGC(SEQ ID NO: 19159) |
| R2042_ncRN | R2042 ncRNA containing 25 | GCGUUAAGGUGGUUAUAUUCUAGUAUUUAUGAAGUGUAGUCGC |
| A_AAVS1 | bp insertion in EMX1 gene | UUCGAUCGUUAAGGCUGAUUUUAACCUCUGCAUAAUAAUAUCG |
| gRNA_25 | fused to AAVSI sgRNA at 3′ | GUAGAUAUUAUUAUGCACGCUCCCUUUAGCAGAGCUAAGAAUC |
| | end | GCUCACUCAGGCACAAGCUUUGAGGUCUGUCCCCUCCACCCCAC |
| | | AGUGGGGCCACUAGGGACAGAAAUGACAGUGGUUGGUGCUCUA |
| | | AAAAUUAAUGACAGAAAAGCCCCAUCCUUAGGCCUCCUCCUUCC |
| | | UAGUCUCCUGAUAUUGGGUCUAACCCCCACCUCAAAGCUUGUGC |
| | | CUGAGUGAGAGCUAAAGAAAAGAAAAGUAGAAUAAGCCACCUU |
| | | AACGCAAUAACAACGGGGCCACUAGGGACAGGAUguuucagag |
| | | cuaugcuggaaacagcauagcaaguugaaauaaggcuaguccg |
| | | uuaucaacuugaaaaaguggcaccgagucggugcuuuuu(SEQ |
| | | ID NO: 19160) |
| R2042_ncRN | R2042 ncRNA containing 25 | GCGUUAAGGUGGUUAUAUUCUAGUAUUUAUGAAGUGUAGUCGC |
| A_EMX1 | bp insertion in EMX1 gene | UUCGAUCGUUAAGGCUGAUUUUAACCUCUGCAUAAUAAUAUCG |
| gRNA_25 | fused to EMX1 sgRNA at 3′ | GUAGAUAUUACUAUGCACGCUCCCUUUAGCAGAGCUAAGAAUC |
| | end | GCUCACUCAGGCACAAGCUUUGAGGACAAACGGCAGAAGCUGGA |
| | | GGAGGAAGGGCCUGAGUCCGAGCAGAAGAAaACUCGAGCUCUGA |
| | | AUGACUCCUAUAAAGUUCUCCCAUCACAUCAACCGGUGGCGCAU |
| | | UGCCACGAAGCAGGCCAAUGGGGAGGACAUCGAUGUCACCUCAA |
| | | AGCUUGUGCCUGAGUGAGAGCUAAAGAAAAGAAAAGUAGAAUA |
| | | AGCCACCUUAACGCAAUAACAACgaguccgagcagaagaagaag |
| | | uuucagagcuaugcuggaaacagcauagcaaguugaaauaaggc |
| | | uaguccguuaucaacuugaaaaaguggcaccgagucggu |
| | | gcuuuuu(SEQ ID NO: 19161) |
| R2042_ncRN | R2042 ncRNA containing 50 | GCGUUAAGGUGGUUAUAUUCUAGUAUUUAUGAAGUGUAGUCGC |
| A_EMX1 | bp insertion in EMX1 gene | UUCGAUCGUUAAGGCUGAUUUUAACCUCUGCAUAAUAAUAUCG |
| gRNA_50 | fused to EMX1 sgRNA at 3′ | GUAGAUAUUAUUAUGCACGCUCCCUUUAGCAGAGCUAAGAAUC |
| | end | GCUCACUCAGGCACAAGCUUUGAGGACAAACGGCAGAAGCUGGA |
| | | GGAGGAAGGGCCUGAGUCCGAGCAGAAGAAaACUCGAGCUCUGA |
| | | UCAGUAUCAUGGGGCCCAUGGUUGAAUGACUCCUAUAaaguuCUC |
| | | CCAUCACAUCAACCGGUGGCGCAUUGCCACGAAGCAGGCCAAUG |
| | | GGGAGGACAUCGAUGUCACCUCAAAGCUUGUGCCUGAGUGAGA |
| | | GCUAAAGAAAAGAAAAGUAGAAUAAGCCACCUUAACGCAAUAA |
| | | CAACgaguccgagcagaagaagaaguuucagagcuaugcuggaa |
| | | acagcauagcaaguugaaauaaggcuaguccguuaucaacuuga |
| | | aaaaguggcaccgagucggugcuuuuu(SEQ ID NO: 19162) |
| R2042_ncRN | R2042 ncRNA containing 75 | GCGUUAAGGUGGUUAUAUUCUAGUAUUUAUGAAGUGUAGUCGC |
| A_EMX1 | bp insertion in EMX1 gene | UUCGAUCGUUAAGGCUGAUUUUAACCUCUGCAUAAUAAUAUCG |
| gRNA_75 | fused to EMX1 sgRNA at 3′ | GUAGAUAUUAUAUGCACGCUCCCUUUAGCAGAGCUAAGAAUC |
| | end | GCUCACUCAGGCACAAGCUUUGAGGACAAACGGCAGAAGCUGGA |
| | | GGAGGAAGGGCCUGAGUCCGAGCAGAAGAAaACUCGAGCUCUGA |
| | | GCCCCACCGCAGCUUGCCAGCACUUUCAGUAUCAUGGGGCCCAU |
| | | GGUUGAAUGACUCCUAUAaaguuCUCCCAUCACAUCAACCGGUGG |
| | | CGCAUUGCCACGAAGCAGGCCAAUGGGGAGGACAUCGAUGUCAC |
| | | CUCAAAGCUUGUGCCUGAGUGAGAGCUAAAGAAAAGAAAAGUA |
| | | GAAUAAGCCACCUUAACGCAAUAACAACgaguccgagcagaagaa |
| | | gaaguuucagagcuaugcuggaaacagcauagcaaguugaaauaa |
| | | ggcuaguccguuaucaacuugaaaaaguggcaccgagucggugcu |
| | | uuuu(SEQ ID NO: 19163) |
| R2042_ncRN | R2042 ncRNA containing 100 | GCGUUAAGGUGGUUAUAUUCUAGUAUUUAUGAAGUGUAGUCGC |
| A_EMX1 | bp insertion in EMX1 gene | UUCGAUCGUUAAGGCUGAUUUUAACCUCUGCAUAAUAAUAUCG |
| gRNA_100 | fused to EMX1 sgRNA at 3′ | GUAGAUAUUACUAUGCACGCUCCCUUUAGCAGAGCUAAGAAUC |
| | end | GCUCACUCAGGCACAAGCUUUGAGGACAAACGGCAGAAGCUGGA |
| | | GGAGGAAGGGCCUGAGUCCGAGCAGAAGAAaACUCGAGCUCUGA |
| | | GCCCCACUGUCGAGAAGUAUGUAUCUCGCUCCCGCAGCUUGCCA |
| | | GCACUUUCAGUAUCAUGGGGCCCAUGGUUGAAUGACUCCUAUAa |
| | | aguuCUCCCAUCACAUCAACCGGUGGCGCAUUGCCACGAAGCAGG |
| | | CCAAUGGGGAGGACAUCGAUGUCACCUCAAAGCUUGUGCCUGAG |
| | | UGAGAGCUAAAGAAAAGAAAAGUAGAAUAAGCCACCUUAACGC |
| | | AAUAACAACgaguccgagcagaagaagaaguuucagagcuaugcu |
| | | ggaaacagcauagcaaguugaaauaaggcuaguccguuaucaacu |
| | | ugaaaaaguggcaccgagucgguguuuuu(SEQ ID NO: 19164) |
| R2042_ncRN | R2042 ncRNA containing 25 | GCGUUAAGGUGGUUAUAUUCUAGUAUUUAUGAAGUGUAGUCGC |
| A_NT_EMX1 | bp insertion (on opposite strand | UUCGAUCGUUAAGGCUGAUUUUAACCUCUGCAUAAUAAUAUCG |
| gRNA_25_dual | of sgRNA cut) in EMX1 gene | GUAGAUAUUAUUAUGCACGCUCCCUUUAGCAGAGCUAAGAAUC |
| guides | fused to EMX1 sgRNA at 3′ | GCUCACUCAGGCACAAGCUUUGAGGugacaucgauguccuccc |
| | end | cauuggccugcuucguggcaaugcgccaccuauaguuagugua |
| | | cugcaacuuUAUAGGAGUCAUUCACCUCAAAGCUUGUGCCUGA |
| | | GUGAGAGCUAAAGAAAAGAAAAGUAGAAUAAGCCACCUUAACG |
| | | CAAUAACAACgaguccgagcagaagaagaaguuucagagcuau |
| | | gcuggaaacagcauagcaaguugaaauaaggcuaguccguuau |
| | | caacuugaaaaaguggcaccgagucggugcuuuuu(SEQ ID |
| | | NO: 19165) |
| R2042_RT | R2042 RT mRNA sequence, | AUGAAGGACGACCAGUACUCUCAGUGGAAGAAGUACUACGAGA |
| | coding sequence only | GCAGGGGCAUCCUGCCCGAGAUCCAGGACAAGCUCCUGAACUAC |
| | | GCCAAGAUCCACAUCGACAACAACACCCCGGUGAUCUUCAACUU |
| | | CGAGCACCUGACCCUGCUGCUGGGCAGGGAGAAGAACUACCUGU |
| | | CCAGCGUGGUGAACAGCCCCGACAGCCACUACAGAAAGUUCAAG |
| | | AUCAAGAAGAGAUCCGGAGGCGAGAGGGAGAUCACCGCUCCCUA |
| | | UCUCAGCCUCCUGGAGAUGCAGUACUGGAUCUACAGGAACAUCC |
| | | UGAUCAACGUGAAGAUCCACUACGCCGCUCACGGCUUCGCUCAG |
| | | GAUAAGAGCAUUAUCACCAACUCCAGGAACCACCUGGGGCAGAA |
| | | ACAUCUGCUCAAGAUGGAUCUGAAGGAUUUCUUCCCCAGCAUCA |
| | | AGCUGAACAGAAUCAUCUACAUCUUCAAGAGCCUGGGCUACCCC |
| | | AAUAUCAUCGCCUUCUACCUGGCCAGCAUCUGCAGCUACAAGGG |
| | | CCACCUGCCCCAGGGCAGCCCCACAAGCCCUAUCCUGAGCAACA |
| | | UCGUGAGCAUCACCCUGGACAACAGACUGGUGAAGUUCGCCAGA |
| | | AAGAUGAAGCUGAGAUACAGCAGGUACGCCGACGACCUGACGUU |
| | | CAGCGGGGACAAGAUCCCCACCAACUACAUCAAGUACAUCACCG |
| | | ACAUCAUCAAUGAUGAGGGCUUCGAGGUGAACGACACCAAGACC |
| | | AAGCUCUACCUGAAGGCCGGGAAGAGAAUCGUGACGGGCAUUUC |
| | | CGUGAUCGGAAAUGACCCGAAGCUUCCGCGGGAAUACAAGCGGA |
| | | AGCUGAAGCAGGAGCUGCACUACAUCUUCACCUACGGCAUCGGC |
| | | AGCCACAUGGCCAAGAAGAAGAUCAAGAAGAUCAACUACCUCUA |
| | | CAGGAUCAUCGGCAAGGUGAACUUCUGGCUGAACAUCGAGCCCG |
| | | ACAACGAGUACGCCAGAAACGCCAAGGCCAAGCUGCUGCUGCUC |
| | | AUCGACAACccaccaaagaagaaaagaaaggucuga(SEQ ID |
| | | NO: 19166) |
| R6943 RT | R6943 RT mRNA sequence, | AUGGAGGAGAGCACCAACUACAAGCUGCUGGUGUGGGGACUGA |
| | coding sequence only | GCGUGAUCCAACCCGCUACCCCCAACGAGGUGCUGAACUACCUC |
| | | ACCAGCACCCUGAACGAUAACGGGCUGCUGCCCGACGUGGAGAA |
| | | GAUGAUCCACUACUUUGAGCUGCUGGACCAGCUGGGCUACAUCC |
| | | ACCAGGUGAGCAAGAGGAACAACCUCUACUCACUGACCCCCAGG |
| | | GGCAACGAAAGGUUGACCCCUGCCCUGAAGAGACUCAGGGACAA |
| | | GAUCAGACUGUUCAUGCUGGACAACUGCCACAGCAUCAGCAAGC |
| | | UGGGCGUGCUGGCCAGCACAGAUACAGAGAACAUGGGGGGCGAC |
| | | AGCCCCUCACUCCAGCUGAGGCACAACCUCAAGGAGGUGCCUCA |
| | | UCCCAGCCUUAGCUGGGCUGCUGGAACCCUGCCUAGCUCUCCUA |
| | | GGCAGGCUUGGGUUCGGAUCUACGAACAGCUGAACAUCGGCAGC |
| | | AUGAGCAGCGACGAGGCUAGCACACCCACCACCGCCAGAAAUGC |
| | | CCCCCUGAGCUUCGUGGGCAGGCUGGGCUUCAGCCUGAACUACU |
| | | ACAGCUUCAACAAGAUCGACGAGCCCCUGUUCAACAACGAUGGC |
| | | GUGACCGCCAUCGCCAGCUGCAUCGGCAUCAGCCCCGGGCUGAU |
| | | UACCGCUAUGGUGAAGUCACCAAAGCGGUACUACAGGACCUUCA |
| | | ACCUGAGAAAGAAGUCCGGGGGCUUCAGAUCCAUUCUGGCCCCC |
| | | AGAAAGUUCAUCAAGACCAUCCAGUACUGGCUGAAGGAUCAUG |
| | | UGCUGAACAGGCUCAAGAUCCACAGCUCCUGUUACAGCUACAGG |
| | | AGCGGCGUGUCCAUCAAGGACAACGCCAUCAACCACGUGAAGAA |
| | | GAAGUUCGUGGCCAGCAUCGACAUUUCCGAUUACUUCGGAAGCA |
| | | UCAACAAGAAGAUGGUGAAGGACUGCUUUUACAAGAACAAUAU |
| | | UCCCGAUCACAUCGUGAAUACCAUCAGCGGCAUCGUGACCUACA |
| | | ACGACGUGCUGCCUCAGGGCGCUCCCACCAGCCCUAUCAUUAGC |
| | | AACGCCAUCCUGUUCGAGUUCGACGAGGAGAUGACGGCUCAUGC |
| | | CCUCACUCUCGACUGUAUCUACACCAGAUACAGCGACGACAUCU |
| | | CGAUAUCCUCCGACUAUAAGGAGAAUAUCGCCAUCCUGAUCAAC |
| | | AUCGCCGAGGCCAACCUGUUGAGCGCUGGAUUCACGCUCAACAG |
| | | ACAGAAGCAAAGGAUUGCUUCUGACAACAGCCGCCAGGUUGUGA |
| | | CCGGCAUCCUGGUGAACGAGAGCAUCAGACCCACCAGAUGCUAC |
| | | AGAAAGAAGAUCAGAAGCGCCUUUGAUCACGCCCUGAAGGAGCA |
| | | GGACGGCUCCCAGCUGACAAUCAACAAGUUGAGGGGCUACCUCA |
| | | ACUACCUGAAGUCCUUCGAGACCUACGGCUUCAAGUUCAACGAG |
| | | AAGAAGUAUAAGGAGACCCUGGAUUUCCUGAUCGCUCUGAAGC |
| | | AGAGCccaccaaagaagaaaagaaaggucuga(SEQ ID NO: |
| | | 19167) |
| R6943_ncRN | R6943 ncRNA containing GFP | GCGGAGUGCUGGCCUCAACUGAUACAGAGAAUAUGGGCGGUGA |
| A_EMX1gR | gene insertion in EMX1 gene | UUCGCCGUCUUUACAGUUAAGGCACAAUUUAAAAGAGGUUCCGC |
| NA_GFP | fused to EMX1 sgRNA at 3′ | ACCCAAGCCUGUCUUGGGCUGCacaaacggcagaagcuggaggag |
| gene | end. Entire GFP cassette is in | gaagggccugaguccgagcagaagaacccauauguccuuccgagu |
| | antisense orientation and | gagagacacaaaaaauuccaacacacuauugcaaugaaaauaaau |
| | contains mini EFla promoter | uuccuuuauuagccagaagucagaugcucaaggggcuucaugaug |
| | and beta globin polyA signal. | uccccauaauuuuuggcagagggaaaaagaucucagugguauuug |
| | | ugagccagggcauuggccacaccagccaccaccuucugauaggca |
| | | gccugcaccugaggagugcggccgcuuuacuuguacagcucgucc |
| | | augccgagagugaucccggcggcggucacgaacuccagcaggacc |
| | | augugaucgcgcuucucguuggggucuuugcucagggggacuggg |
| | | ugcucagguagugguugucgggcagcagcacggggccgucgccga |
| | | uggggguguucugcugguaguggucggcgagcugcacgcugccgu |
| | | ccucgauguuguggggagaugcgguucaccagggugucgccccga |
| | | acuucaccuggggggggccuugaagaagauggugcgcuccuggac |
| | | guagccuucgggcauggcggacuugaagaagucgugcugcuucau |
| | | guggucgggguagcggcugaagcacugcacgccguaggucagggu |
| | | ggucacgagggugggccagggcacgggcagcuugccgguggugca |
| | | gaugaacuucagggucagcuugccguagguggcaucgcccucgcc |
| | | cucgccggacacgcugaacuuguggccguuuacgucgccguccag |
| | | cucgaccaggaugggcaccaccccggugaacagcuccucgcccuu |
| | | gcucaccaugguggcgaccgguggaucccgggcccgcgguaccgu |
| | | cgacugcagaauucgaagcuugagcucgagaucugaguccgguag |
| | | cgcuagcggaucugacgguucacuaaacccuguguucuggcggca |
| | | aacccguugcgaaaaagaacguucacggcgacuacugcacuuaua |
| | | uacgguucucccccacccucgggaaaaaggcggagccaguacacg |
| | | acaacuucucggggacugugggcgaugugcgcucugcccguucuc |
| | | ccaucacaucaaccgguggcgcauugccacgaagcaggccaaugg |
| | | ggaggacaucgaugucaGCAGCCCAAGACAGGCUUGGGUGCGGAU |
| | | CUACGAGCAAUUAAAUAUUGGUUCGAUGUCCAGUGAUGAGGCCAG |
| | | CACCCGCAAUAACAACgaguccgagcagaagaagaaguuucagag |
| | | cuaugcuggaaacagcauagcaaguugaaauaaggcuaguccguu |
| | | aucaacuugaaaaaguggcaccgagucggugcuuuuu(SEQ ID |
| | | NO: 19168) |
| R6943_ncRN | R6943 ncRNA containing 100 | GCGGAGUGCUGGCCUCAACUGAUACAGAGAAUAUGGGCGGUGA |
| A_EMX1gR | bp insertion in EMX1 gene | UUCGCCGUCUUUACAGUUAAGGCACAAUUUAAAAGAGGUUCCGC |
| NA_100bp | fused to EMX1 sgRNA at 3′ | ACCCAAGCCUGUCUUGGGCUGCacaaacggcagaagcuggaggaggaagggccug |
| | end | aguccgagcagaagaaaACUCGAGCUCUGAGCCCCACUGUCGAGAAGUAU |
| | | GUAUCUCGCUCCCGCAGCUUGCCAGCACUUUCAGUAUCAUGGGG |
| | | CCCAUGGUUGAAUGACUCCUAUAaaguucucccaucacaucaaccgguggcgcau |
| | | ugccacgaagcaggccaauggggaggacaucgaugucaGCAGCCCAAGACAGGCUUG |
| | | GGUGCGGAUCUACGAGCAAUUAAAUAUUGGUUCGAUGUCCAGU |
| | | GAUGAGGCCAGCACCCGCAAUAACAACgaguccgagcagaagaagaaguuuca |
| | | gagcuaugcuggaaacagcauagcaaguugaaauaaggcuaguccguuaucaa |
| | | cuugaaaaaguggcaccgagucggugcuuuuu(SEQ ID NO: 19169) |
| R6943_ncRN | R6943 ncRNA containing 75 | GCGGAGUGCUGGCCUCAACUGAUACAGAGAAUAUGGGCGGUGA |
| A_EMX1gR | bp insertion in EMX1 gene | UUCGCCGUCUUUACAGUUAAGGCACAAUUUAAAAGAGGUUCCGCACCCA |
| NA_75bp | fused to EMX1 sgRNA at 3′ | AGCCUGUCUUGGGCUGCacaaacggcagaagcuggaggaggaagggccug |
| | end | aguccgagcagaagaaaACUCGAGCUCUGAGCCCCACCGCAGCUUGCCAG |
| | | CACUUUCAGUAUCAUGGGGCCCAUGGUUGAAUGACUCCUAUAaaguucuc |
| | | ccaucacaucaaccgguggcgcauugccacgaagcaggccaauggggagg |
| | | acaucgaugucaGCAGCCCAAGACAGGCUUGGGUGCGGAUCUACGAGCAA |
| | | UUAAAUAUUGGUUCGAUGUCCAGUGAUGAGGCCAGCACCCGCAAUAACAA |
| | | Cgaguccgagcagaagaagaaguuucagagcuaugcuggaaacagcauag |
| | | caaguugaaauaaggcuaguccguuaucaacuugaaaaaguggcaccgag |
| | | ucgguguuuuu(SEQ ID NO: 19170) |
| R6943_ncRN | R6943 ncRNA containing 50 | GCGGAGUGCUGGCCUCAACUGAUACAGAGAAUAUGGGCGGUGA |
| A_EMX1gR | bp insertion in EMX1 gene | UUCGCCGUCUUUACAGUUAAGGCACAAUUUAAAAGAGGUUCCGC |
| NA_50bp | fused to EMX1 sgRNA at 3′ | ACCCAAGCCUGUCUUGGGCUGCacaaacggcagaagcuggaggaggaagggccug |
| | end | aguccgagcagaagaaaACUCGAGCUCUGAUCAGUAUCAUGGGGCCCAUGGU |
| | | UGAAUGACUCCUAUAaaguucucccaucacaucaaccgguggcgcauugccacga |
| | | agcaggccaauggggaggacaucgaugucaGCAGCCCAAGACAGGCUUGGGUGC |
| | | GGAUCUACGAGCAAUUAAAUAUUGGUUCGAUGUCCAGUGAUGA |
| | | GGCCAGCACCCGCAAUAACAACgaguccgagcagaagaagaaguuucagagcuau |
| | | gcuggaaacagcauagcaaguugaaauaaggcuaguccguuaucaacuugaaaaa |
| | | guggcaccgagucggugcuuuuu(SEQ ID NO: 19171) |
| R6943_ncRN | R6943 ncRNA containing 25 | GCGGAGUGCUGGCCUCAACUGAUACAGAGAAUAUGGGCGGUGAUUCGCCG |
| A_EMX1gR | bp insertion in EMX1 gene | UCUUUACAGUUAAGGCACAAUUUAAAAGAGGUUCCGCACCCAAGCCUGUC |
| NA_25bp | fused to EMX1 sgRNA at 3′ | UUGGGCUGCacaaacggcagaagcuggaggaggaagggccugaguccgag |
| | end | cagaagaaaACUCGAGCUCUGAAUGACUCCUAUAaaguucucccaucaca |
| | | ucaaccgguggcgcauugccacgaagcaggccaauggggaggacaucgau |
| | | gucaGCAGCCCAAGACAGGCUUGGGUGCGGAUCUACGAGCAAUUAAAUAU |
| | | UGGUUCGAUGUCCAGUGAUGAGGCCAGCACCCGCAAUAACAACgaguccg |
| | | agcagaagaagaaguuucagagcuaugcuggaaacagcauagcaaguuga |
| | | aauaaggcuaguccguuaucaacuugaaaaaguggcaccgagucggugcu |
| | | uuuu(SEQ ID NO: 19172) |
| R6943_ncRN | R6943 ncRNA containing 25 | GCGGAGUGCUGGCCUCAACUGAUACAGAGAAUAUGGGCGGUGA |
| A_AAVSI | bp insertion in AAVSI gene | UUCGCCGUCUUUACAGUUAAGGCACAAUUUAAAAGAGGUUCCGC |
| gRNA_25bp | fused to AAVSI sgRNA at 3′ | ACCCAAGCCUGUCUUGGGCUGCUCUGUCCCCUCCACCCCACAGU |
| | end | GGGGCCACUAGGGACAGAAAUGACAGUGGUUGGUGCUCUAAAA |
| | | AUUAAUGACAGAAAAGCCCCAUCCUUAGGCCUCCUCCUUCCUAG |
| | | UCUCCUGAUAUUGGGUCUAACCCCCAGCAGCCCAAGACAGGCUU |
| | | GGGUGCGGAUCUACGAGCAAUUAAAUAUUGGUUCGAUGUCCAG |
| | | UGAUGAGGCCAGCACCCGCAAUAACAACGGGGCCACUAGGGACAGGAUguuuc |
| | | agagcuaugcuggaaacagcauagcaaguugaaauaaggcuaguccguuauca |
| | | acuugaaaaaguggcaccgagucggugcuuuuu(SEQ ID NO: 19173) |
| R6943_ncRN | R6943 ncRNA containing 25 | GCGGAGUGCUGGCCUCAACUGAUACAGAGAAUAUGGGCGGUGA |
| A only_25bp | bp insertion in EMX1 gene | UUCGCCGUCUUUACAGUUAAGGCACAAUUUAAAAGAGGUUCCGC |
| | | ACCCAAGCCUGUCUUGGGCUGCacaaacggcagaagcuggaggaggaagggccug |
| | | aguccgagcagaagaaaACUCGAGCUCUGAAUGACUCCUAUAaaguucucccau |
| | | cacaucaaccgguggcgcauugccacgaagcaggccaauggggaggacaucgaug |
| | | ucaGCAGCCCAAGACAGGCUUGGGUGCGGAUCUACGAGCAAUUAAAUAUUG |
| | | GUUCGAUGUCCAGUGAUGAGGCCAGCACCCGC(SEQ ID NO: 19174) |
| AAVS1 | Contains chemical | GGGGCCACUAGGGACAGGAUguuucagagcuaugcuggaaacagcauagcaaguug |
| sgRNA | modifications from Synthego: | aaauaaggcuaguccguuaucaacuugaaaaaguggcaccgagucggugcuuuuu |
| | 2′-F, 2′-O-methyl, | (SEQ ID NO: 19175) |
| | phosphorothioates | |
| R1262_RT-1 | R1262 retron RT mRNA | AGGAUAAUGGGAAAUAAGAGAGAAAAGAAGAGUAAGAAGAAAU |
| (R1262 RT) | | AUAAGAGCCACCAUGAUCAGCUUCAGCGAGAUCAAGAGCAGAAA |
| | | CGAUUUUGCAGACGCUCUGCAGAUUCCUCGGAGCGUGCUGACGC |
| | | ACGUUCUGUACAUUGCCAAGCCCGAGAGCUUCUACGAGAGCUUC |
| | | ACCAUCCCCAAGAAGAAUGGGGAGGACAGAAUCAUCAUGGCCCC |
| | | CAAGGGCACCCUGAAGUCCAUCCAGACCAAGCUGAGCAAGCAGC |
| | | UGGUGGAGUACAGAGCCUCCAUCAGCCAGAAAGGCCAGGAGAAG |
| | | UCCAACAUCUCCCAUGGCUUCGAGAGGGAGAAGUCCAUCAUCAC |
| | | CAAUGCUCAGAUACACCGCAACAAGCGGUAUGUCAUCAACUACG |
| | | ACCUGAAGGACUUCUUCGACUCCUUCCACUUCGGCAGAGUGGUG |
| | | GGAUUCUUCGAGAAGAACAAGCACUUCCUGCUGCCCUACGAGGU |
| | | GGCCGUGAUCAUCGCCCAGCUGACCUGCUAUAAUGGCAGGCUGC |
| | | CCCAGGGCGCCCCCACAAGCCCUGUGAUCACCAACCUGAUCUGC |
| | | GAGAUCCUGGACUACAGGGUGCUCAAGAUCGCCAAGAGAUACAA |
| | | GCUGGACUACACCAGGUACGCUGACGACCUGACCUUCAGCACCA |
| | | ACUACUCCAGAUUCCUGGAGGUGUUCGACAGCUUCGCCAAGGAG |
| | | CUGCUCCAGGAGAUCAGCAACUCUGGUUUUACCAUAAACCAGAG |
| | | CAAGACCAGGCUGCUGUACAGAGACAGCCGCCAGGAGGUCACAG |
| | | GCCUCGUGGUGAAUAAGAAGAUCGGCGUGAACAGAGAGUACGU |
| | | CAAGAGCACUAGGGCGAUGGCCCAGGCUCUGUACAGCACCGGCG |
| | | AGUUCACCAUCAACGGCAUCCCCGGCACCAUCAAACAGCUGGAG |
| | | GGCAGGUUCGGCUUCAUCGACCAGCUGGACCACUACAACAACGU |
| | | GAUCGACGAUGCCAAGCACGACGCCUACUCCCUGAACGGCAGGG |
| | | AGAAGCAGUUCCAGGAGUUCCUGUUCUUCAAGACAUUCUUCUUC |
| | | AACGAGUACCCCCUGGUGAUCACCGAGGGCAAGACCGACAUCAG |
| | | AUACCUGAAGGCCGCCCUCAAGAGCCUGCACCAGAAGUACCCCG |
| | | AGCUGAUCUGCAAGGAGGACGACGGAACCUUUCGGUUCAAGAUC |
| | | AGCUUCUUCAGAAGAUCCAAGAGAUGGAAGUACUUCUUCGGCAU |
| | | CAGCAAGGAUGGCGCUGAUGCCAUGAAGCUGCUGUACCGGUUCU |
| | | UUACCGGACAGAAGGGCGUGAAGAACUACUACAGGCUGUUCGCC |
| | | GAGAAGUACAAGGCCGUGCAGAGAAACCCCGUGAUCAUGCUGUU |
| | | UGACAACGAGAUGGAGAGCAAGAGACCCCUCAACAAGUUCAUCU |
| | | CCGAGGAGGUGAAGAUUCCCUCCUCGGAGCAGCAGCUGUUCAAG |
| | | GAGCAGCUGUACUACCACCUGAUCCCCGGCAGCAAGACCUACCU |
| | | GAUGACCCACCCCUUGCCUCCUGGCAAGACAGAGGCCGAGAUCG |
| | | AGGACCUGUUCCCUACCGAGGUGUUGGGCGUGAAGCUCGACGGC |
| | | AAGAGCUUCAGCACCAAGGACAAGUUCGACACCAGCAAGUUCUA |
| | | CGGCAAGGACAUCUUCAGCAGCUACGUGUACGAGCACUGGAAGU |
| | | CCAUCGACUUCAGCGGCUUCAUCCCCCUGCUGGACAAGAUCAAC |
| | | AUGCUCGUGCAGAACGAGAAGAAGCCCGGGCUGAACACACCACC |
| | | AAAGAAGAAAAGAAAGGUCUGAGCUGGAGCCUCGGUGGCCAUGC |
| | | UUCUUGCCCCUUGGGCCUCCCCCCAGCCCCUCCUCCCCUUCCUG |
| | | CACCCGUACCCCCGUGGUCUUUGAAUAAAGUCUGAGUGGGCGGC |
| | | GAU (SEQ ID NO: 19176) |
| R1262_nc-5 | R1262 ncRNA containing 505 | GGGAUAAUGAGCGUGUGACAAGAUUUUGGGCUUGUGUUUCGCA |
| (R1262_ncR | bp insertion in EMX1 site | AGCUUUGAUUAAAAUGGUAGAUGGAUUUGCUAUCUAUCGUCAU |
| NA- | | UUCCAGUACUGUUAUGUACAAACGGCAGAAGCUGGAGGAGGAA |
| EMX1_505) | | GGGCCUGAGUCCGAGCAGAAGAAACUUCAGCUAAGGAAGCUACC |
| | | AAUAUUUAGUUUCUGAGUCUCACGACAGACCUCGCGCGUAGAUU |
| | | GCCAUGCGUAGAGCUAACGAGCCAGCGGAAAGCGUGAGGCGCUU |
| | | UUAAGCAUGGCGAGUAAGUGAUCCAACGCUUCGGAUAUGACUA |
| | | UAUACUUAGGUUCGAUCUCGUCCCGAGAAUUCUAAGCCUCAACA |
| | | UCUAUGAGUUAUGAGGUUAGCCGAAAAAGCACGUGGUGGCGCCC |
| | | ACCGACUGUUCCCAGACUGUAGCUCUUUGUUCUGUCAAGGCCCG |
| | | ACCUUCAUCGCGGCCGAUUCCUUCUGCGGACCAUACCGUCCUGA |
| | | UACUUUGGUCAUGUUUCCGUUGUAGGAGUGAACCCACUUGCCUU |
| | | UGCGUCUUAAUACCAAUGAAAAACCUAUGCACUUUGUACAGGGU |
| | | ACCAUCGGGAUUCUGAACCCUCAGAUAGUGGGGAUCCCGGGUAU |
| | | AGACCUUUAUCUGCGGUCCAACUUAGGCAUAAACCUCCAUGCUA |
| | | CAAAGUUCUCCCAUCACAUCAACCGGUGGCGCAUUGCCACGAAG |
| | | CAGGCCAAUGGGGAGGACAUCGAUGUCAGCAUAAUGAGUUGUC |
| | | ACACGCUCGAU (SEQ ID NO: 19177) |
| R1262_nc-3 | R1262 ncRNA containing 305 | GGGAUAAUGAGCGUGUGACAAGAUUUUGGGCUUGUGUUUCGCA |
| (R1262_ncR | bp insertion in EMX1 site | AGCUUUGAUUAAAAUGGUAGAUGGAUUUGCUAUCUAUCGUCAU |
| NA- | | UUCCAGUACUGUUAUGUACAAACGGCAGAAGCUGGAGGAGGAA |
| EMX1_305) | | GGGCCUGAGUCCGAGCAGAAGAACGACUACCAAAUCCGCAUGUU |
| | | ACGGGACUUCUUAUUAAUUCUUUUUUCGUGAGGAGCAGCGGAU |
| | | CUUAAUGGAUGGCCGCAGGUGGUAUGGAAGCUAAUAGCGCGGG |
| | | UGAGAGGGUAAUCAGCCGUGUCCACCAACACAACGCUAUCGGGC |
| | | GAUUCUAUAAGAUUCCGCAUUGCGUCUACUUAUAAGAUGUCUCA |
| | | ACGGUAUCCGCAACUUGCGAUGUGCCUGCUAUCCUUAAAUGCAU |
| | | AUCUCGCCCAGUAGCUUCCCAAUAUGAGAGCAUCAAUUGUAGAU |
| | | CGGGCCGGGAUAGUCAUGUCGUAAAGUUCUCCCAUCACAUCAAC |
| | | CGGUGGCGCAUUGCCACGAAGCAGGCCAAUGGGGAGGACAUCGA |
| | | UGUCAGCAUAAUGAGUUGUCACACGCUCGAU (SEQ ID NO: 19178) |
| R1262_nc-1 | R1262 ncRNA containing 25 bp | GGGAUAAUGAGCGUGUGACAAGAUUUUGGGCUUGUGUUUCGCA |
| (R1262_ncR | insertion in EMX1 site | AGCUUUGAUUAAAAUGGUAGAUGGAUUUGCUAUCUAUCGUCAU |
| NA- | | UUCCAGUACUGUUAUGUACAAACGGCAGAAGCUGGAGGAGGAA |
| EMX1_25) | | GGGCCUGAGUCCGAGCAGAAGAAAACUCGAGCUCUGAAUGACUC |
| | | CUAUAAAGUUCUCCCAUCACAUCAACCGGUGGCGCAUUGCCACG |
| | | AAGCAGGCCAAUGGGGAGGACAUCGAUGUCAGCAUAAUGAGUU |
| | | GUCACACGCUCGAU (SEQ ID NO: 19179) |
| R1262_nc-4 | R1262 ncRNA containing 405 | GGGAUAAUGAGCGUGUGACAAGAUUUUGGGCUUGUGUUUCGCA |
| (R1262_ncR | bp insertion in EMX1 site | AGCUUUGAUUAAAAUGGUAGAUGGAUUUGCUAUCUAUCGUCAU |
| NA- | | UUCCAGUACUGUUAUGUACAAACGGCAGAAGCUGGAGGAGGAA |
| EMX1_405) | | GGGCCUGAGUCCGAGCAGAAGAACUGCUAAAUCCGCGUGAUAGG |
| | | GGAUUUGAAGUUUAAUCUUCUAUCGCAAGGAACUGCCGAUCUU |
| | | AAUGGAUGGCCGGAGGUGGUAUGGAAGCUAUAAGCGCGGGUGA |
| | | GAGGGUAAUUAGGCGUGUUCACCUACGCUACGCUAACGGGCGAU |
| | | UCUAUAAGAUUGCACAUUGCGUCAACUCAUAAGAUGUCUCAACG |
| | | GCAUGCGCAACUUGUGAAGUGUCUACUAUCCUUAAACGCAUAUC |
| | | UCGCACAGUAACUCCCGAAUAUGUCGGCAUCUGAUGUUGCCCGG |
| | | GCCGAGUUAGUGUUGAGCUCACGGAACUUAUUGUAUGAGUAGU |
| | | GAUUUGUAAGAGUUGUCAGUUAGCUCGUUCAGGUAAUAGUUGC |
| | | CCACACAACGUCAAAAUAAGAGAACGGUCGUAACAUAAAGUUCU |
| | | CCCAUCACAUCAACCGGUGGCGCAUUGCCACGAAGCAGGCCAAU |
| | | GGGGAGGACAUCGAUGUCAGCAUAAUGAGUUGUCACACGCUCGA |
| | | U (SEQ ID NO: 19180) |
| R1262_nc-2 | R1262 ncRNA containing 205 | GGGAUAAUGAGCGUGUGACAAGAUUUUGGGCUUGUGUUUCGCA |
| (R1262_ncR | bp insertion in EMX1 site | AGCUUUGAUUAAAAUGGUAGAUGGAUUUGCUAUCUAUCGUCAU |
| NA- | | UUCCAGUACUGUUAUGUACAAACGGCAGAAGCUGGAGGAGGAA |
| EMX1_205) | | GGGCCUGAGUCCGAGCAGAAGAAAAAGCGGUCUUACGGUCAGUC |
| | | GUAUUCCUUCUCGAGUUCCGUCCAGUUGAGCGUGUCACUCCCAG |
| | | UGUACCUGCAAGCCGAGAUGGCUGUGCUUGGAGUCAAUCGCAUG |
| | | UAGGAUGGUCUCCAGACACCGGGGCACCAGUUUUCACGCCUAAA |
| | | GCAUAAACGACGAGCAGUCAUGAAAGUCUUAGUACUGGACGUGC |
| | | CGUUUCACAAAGUUCUCCCAUCACAUCAACCGGUGGCGCAUUGC |
| | | CACGAAGCAGGCCAAUGGGGAGGACAUCGAUGUCAGCAUAAUGA |
| | | GUUGUCACACGCUCGAU (SEQ ID NO: 19181) |
| R1262_nc-19 | R1262 ncRNA containing 205 | GGGAUAAUGAGCGUGUGACAAGAUUUUGGGCUUGUGUUUCGCA |
| (R1262_ncR | bp insertion in EMX1 site | AGCUUUGAUUAAAAUGGUAGAUGGAUUUGCUAUCUAUCGUCAU |
| NA- | combined with EMX1 sgRNA | UUCCAGUACUGUUAUGUACAAACGGCAGAAGCUGGAGGAGGAA |
| sgEMX1_205) | at 3′ end | GGGCCUGAGUCCGAGCAGAAGAAAAAGCGGUCUUACGGUCAGUC |
| | | GUAUUCCUUCUCGAGUUCCGUCCAGUUGAGCGUGUCACUCCCAG |
| | | UGUACCUGCAAGCCGAGAUGGCUGUGCUUGGAGUCAAUCGCAUG |
| | | UAGGAUGGUCUCCAGACACCGGGGCACCAGUUUUCACGCCUAAA |
| | | GCAUAAACGACGAGCAGUCAUGAAAGUCUUAGUACUGGACGUGC |
| | | CGUUUCACAAAGUUCUCCCAUCACAUCAACCGGUGGCGCAUUGC |
| | | CACGAAGCAGGCCAAUGGGGAGGACAUCGAUGUCAGCAUAAUGA |
| | | GUUGUCACACGCUCAAUAACAACGAGUCCGAGCAGAAGAAGAAG |
| | | UUUCAGAGCUAUGCUGGAAACAGCAUAGCAAGUUGAAAUAAGG |
| | | CUAGUCCGUUAUCAACUUGAAAAAGUGGCACCGAGUCGGUGCUU |
| | | UUUGAU (SEQ ID NO: 19182) |
| R1262_nc-20 | R1262 ncRNA containing 205 | GGGAUAAUGAGUCCGAGCAGAAGAAGAAGUUUCAGAGCUAUGC |
| (R1262_sgE | bp insertion in EMX1 site | UGGAAACAGCAUAGCAAGUUGAAAUAAGGCUAGUCCGUUAUCA |
| MX1- | combined with EMX1 sgRNA | ACUUGAAAAAGUGGCACCGAGUCGGUGCUUUUUAAUAACAACG |
| ncRNA_205) | at 5′ end | AGCGUGUGACAAGAUUUUGGGCUUGUGUUUCGCAAGCUUUGAU |
| | | UAAAAUGGUAGAUGGAUUUGCUAUCUAUCGUCAUUUCCAGUAC |
| | | UGUUAUGUACAAACGGCAGAAGCUGGAGGAGGAAGGGCCUGAG |
| | | UCCGAGCAGAAGAAAAAGCGGUCUUACGGUCAGUCGUAUUCCUU |
| | | CUCGAGUUCCGUCCAGUUGAGCGUGUCACUCCCAGUGUACCUGC |
| | | AAGCCGAGAUGGCUGUGCUUGGAGUCAAUCGCAUGUAGGAUGG |
| | | UCUCCAGACACCGGGGCACCAGUUUUCACGCCUAAAGCAUAAAC |
| | | GACGAGCAGUCAUGAAAGUCUUAGUACUGGACGUGCCGUUUCAC |
| | | AAAGUUCUCCCAUCACAUCAACCGGUGGCGCAUUGCCACGAAGC |
| | | AGGCCAAUGGGGAGGACAUCGAUGUCAGCAUAAUGAGUUGUCA |
| | | CACGCUCGAU (SEQ ID NO: 19183) |
| R1262_nc-13 | R1262 ncRNA containing 25 bp | GGGAUAAUGAGCGUGUGACAAGAUUUUGGGCUUGUGUUUCGCA |
| (R1262_ncR | insertion in AAVSI site | AGCUUUGAUUAAAAUGGUAGAUGGAUUUGCUAUCUAUCGUCAU |
| NA- | | UUCCAGUACUGUUAUGUUCUGUCCCCUCCACCCCACAGUGGGGC |
| AAVS1_25) | | CACUAGGGACAGAAAGCGGUCUUACGGUCAGUCGUAUUCCUUCU |
| | | CGAGUUCCGUCCAGUUGAGCGUGUCACUCCCAGUGUACCUGCAA |
| | | GCCGAGAUGGCUGUGCUUGGAGUCAAUCGCAUGUAGGAUGGUC |
| | | UCCAGACACCGGGGCACCAGUUUUCACGCCUAAAGCAUAAACGA |
| | | CGAGCAGUCAUGAAAGUCUUAGUACUGGACGUGCCGUUUCACAA |
| | | UUAAUGACAGAAAAGCCCCAUCCUUAGGCCUCCUCCUUCCUAGU |
| | | CUCCUGAUAUUGGGUCUAACCCCCAGCAUAAUGAGUUGUCACAC |
| | | GCUCGAU (SEQ ID NO: 19184) |
| R1262_nc-14 | R1262 ncRNA containing 205 | GGGAUAAUGAGCGUGUGACAAGAUUUUGGGCUUGUGUUUCGCA |
| (R1262_ncR | bp insertion in AAVSI site | AGCUUUGAUUAAAAUGGUAGAUGGAUUUGCUAUCUAUCGUCAU |
| NA- | | UUCCAGUACUGUUAUGUUCUGUCCCCUCCACCCCACAGUGGGGC |
| AAVS1_205) | | CACUAGGGACAGAAAGCGGUCUUACGGUCAGUCGUAUUCCUUCU |
| | | CGAGUUCCGUCCAGUUGAGCGUGUCACUCCCAGUGUACCUGCAA |
| | | GCCGAGAUGGCUGUGCUUGGAGUCAAUCGCAUGUAGGAUGGUC |
| | | UCCAGACACCGGGGCACCAGUUUUCACGCCUAAAGCAUAAACGA |
| | | CGAGCAGUCAUGAAAGUCUUAGUACUGGACGUGCCGUUUCACAA |
| | | UUAAUGACAGAAAAGCCCCAUCCUUAGGCCUCCUCCUUCCUAGU |
| | | CUCCUGAUAUUGGGUCUAACCCCCAGCAUAAUGAGUUGUCACAC |
| | | GCUCGAU (SEQ ID NO: 19185) |
| R1262_nc-15 | R1262 ncRNA containing 505 | GGGAUAAUGAGCGUGUGACAAGAUUUUGGGCUUGUGUUUCGCA |
| (R1262_ncR | bp insertion in AAVSI site | AGCUUUGAUUAAAAUGGUAGAUGGAUUUGCUAUCUAUCGUCAU |
| NA- | | UUCCAGUACUGUUAUGUUCUGUCCCCUCCACCCCACAGUGGGGC |
| AAVS1_505) | | CACUAGGGACAGACUUCAGCUAAGGAAGCUACCAAUAUUUAGUU |
| | | UCUGAGUCUCACGACAGACCUCGCGCGUAGAUUGCCAUGCGUAG |
| | | AGCUAACGAGCCAGCGGAAAGCGUGAGGCGCUUUUAAGCAUGGC |
| | | GAGUAAGUGAUCCAACGCUUCGGAUAUGACUAUAUACUUAGGU |
| | | UCGAUCUCGUCCCGAGAAUUCUAAGCCUCAACAUCUAUGAGUUA |
| | | UGAGGUUAGCCGAAAAAGCACGUGGUGGCGCCCACCGACUGUUC |
| | | CCAGACUGUAGCUCUUUGUUCUGUCAAGGCCCGACCUUCAUCGC |
| | | GGCCGAUUCCUUCUGCGGACCAUACCGUCCUGAUACUUUGGUCA |
| | | UGUUUCCGUUGUAGGAGUGAACCCACUUGCCUUUGCGUCUUAAU |
| | | ACCAAUGAAAAACCUAUGCACUUUGUACAGGGUACCAUCGGGAU |
| | | UCUGAACCCUCAGAUAGUGGGGAUCCCGGGUAUAGACCUUUAUC |
| | | UGCGGUCCAACUUAGGCAUAAACCUCCAUGCUACAAUUAAUGAC |
| | | AGAAAAGCCCCAUCCUUAGGCCUCCUCCUUCCUAGUCUCCUGAU |
| | | AUUGGGUCUAACCCCCAGCAUAAUGAGUUGUCACACGCUCGAU |
| | | (SEQ ID NO: 19186) |
| R1262_nc-18 | R1262 ncRNA containing 505 | GGGAUAAUGAGCGUGUGACAAGAUUUUGGGCUUGUGUUUCGCA |
| (R1262_ncR | bp insertion in AAVSI site and | AGCUUUGAUUAAAAUGGUAGAUGGAUUUGCUAUCUAUCGUCAU |
| NA-AAVSI- | MS2 stem loop at 3′ end | UUCCAGUACUGUUAUGUUCUGUCCCCUCCACCCCACAGUGGGGC |
| MS2_505) | | CACUAGGGACAGACUUCAGCUAAGGAAGCUACCAAUAUUUAGUU |
| | | UCUGAGUCUCACGACAGACCUCGCGCGUAGAUUGCCAUGCGUAG |
| | | AGCUAACGAGCCAGCGGAAAGCGUGAGGCGCUUUUAAGCAUGGC |
| | | GAGUAAGUGAUCCAACGCUUCGGAUAUGACUAUAUACUUAGGU |
| | | UCGAUCUCGUCCCGAGAAUUCUAAGCCUCAACAUCUAUGAGUUA |
| | | UGAGGUUAGCCGAAAAAGCACGUGGUGGCGCCCACCGACUGUUC |
| | | CCAGACUGUAGCUCUUUGUUCUGUCAAGGCCCGACCUUCAUCGC |
| | | GGCCGAUUCCUUCUGCGGACCAUACCGUCCUGAUACUUUGGUCA |
| | | UGUUUCCGUUGUAGGAGUGAACCCACUUGCCUUUGCGUCUUAAU |
| | | ACCAAUGAAAAACCUAUGCACUUUGUACAGGGUACCAUCGGGAU |
| | | UCUGAACCCUCAGAUAGUGGGGAUCCCGGGUAUAGACCUUUAUC |
| | | UGCGGUCCAACUUAGGCAUAAACCUCCAUGCUACAAUUAAUGAC |
| | | AGAAAAGCCCCAUCCUUAGGCCUCCUCCUUCCUAGUCUCCUGAU |
| | | AUUGGGUCUAACCCCCAGCAUAAUGAGUUGUCACACGCUCACAU |
| | | GAGGAUCACCCAUGUGAU (SEQ ID NO: 19187) |
| R1262_nc-17 | R1262 ncRNA containing 205 | GGGAUAAUGAGCGUGUGACAAGAUUUUGGGCUUGUGUUUCGCA |
| (R1262_ncR | bp insertion in AAVSI site and | AGCUUUGAUUAAAAUGGUAGAUGGAUUUGCUAUCUAUCGUCAU |
| NA-AAVSI- | MS2 stem loop at 3′ end | UUCCAGUACUGUUAUGUUCUGUCCCCUCCACCCCACAGUGGGGC |
| MS2_205) | | CACUAGGGACAGAAAGCGGUCUUACGGUCAGUCGUAUUCCUUCU |
| | | CGAGUUCCGUCCAGUUGAGCGUGUCACUCCCAGUGUACCUGCAA |
| | | GCCGAGAUGGCUGUGCUUGGAGUCAAUCGCAUGUAGGAUGGUC |
| | | UCCAGACACCGGGGCACCAGUUUUCACGCCUAAAGCAUAAACGA |
| | | CGAGCAGUCAUGAAAGUCUUAGUACUGGACGUGCCGUUUCACAA |
| | | UUAAUGACAGAAAAGCCCCAUCCUUAGGCCUCCUCCUUCCUAGU |
| | | CUCCUGAUAUUGGGUCUAACCCCCAGCAUAAUGAGUUGUCACAC |
| | | GCUCACAUGAGGAUCACCCAUGUGAU (SEQ ID NO: 19188) |
| R1262_nc-16 | R1262 ncRNA containing 25 bp | GGGAUAAUGAGCGUGUGACAAGAUUUUGGGCUUGUGUUUCGCA |
| (R1262_ncR | insertion in AAVSI site and | AGCUUUGAUUAAAAUGGUAGAUGGAUUUGCUAUCUAUCGUCAU |
| NA-AAVSI- | MS2 stem loop at 3′ end | UUCCAGUACUGUUAUGUUCUGUCCCCUCCACCCCACAGUGGGGC |
| MS2_25) | | CACUAGGGACAGAACUCGAGCUCUGAAUGACUCCUAUAAUUAAU |
| | | GACAGAAAAGCCCCAUCCUUAGGCCUCCUCCUUCCUAGUCUCCU |
| | | GAUAUUGGGUCUAACCCCCAGCAUAAUGAGUUGUCACACGCUCA |
| | | CAUGAGGAUCACCCAUGUGAU (SEQ ID NO: 19189) |
| R1262_nc-9 | R1262 ncRNA containing 305 | GGGAUAAUGAGCGUGUGACAAGAUUUUGGGCUUGUGUUUCGCA |
| (R1262_ncR | bp insertion in EMX1 site and | AGCUUUGAUUAAAAUGGUAGAUGGAUUUGCUAUCUAUCGUCAU |
| NA-EMX1- | MS2 stem loop at 3′ end | UUCCAGUACUGUUAUGUACAAACGGCAGAAGCUGGAGGAGGAA |
| MS2_305) | | GGGCCUGAGUCCGAGCAGAAGAACGACUACCAAAUCCGCAUGUU |
| | | ACGGGACUUCUUAUUAAUUCUUUUUUCGUGAGGAGCAGCGGAU |
| | | CUUAAUGGAUGGCCGCAGGUGGUAUGGAAGCUAAUAGCGCGGG |
| | | UGAGAGGGUAAUCAGCCGUGUCCACCAACACAACGCUAUCGGGC |
| | | GAUUCUAUAAGAUUCCGCAUUGCGUCUACUUAUAAGAUGUCUCA |
| | | ACGGUAUCCGCAACUUGCGAUGUGCCUGCUAUCCUUAAAUGCAU |
| | | AUCUCGCCCAGUAGCUUCCCAAUAUGAGAGCAUCAAUUGUAGAU |
| | | CGGGCCGGGAUAGUCAUGUCGUAAAGUUCUCCCAUCACAUCAAC |
| | | CGGUGGCGCAUUGCCACGAAGCAGGCCAAUGGGGAGGACAUCGA |
| | | UGUCAGCAUAAUGAGUUGUCACACGCUCACAUGAGGAUCACCCA |
| | | UGUGAU (SEQ ID NO: 19190) |
| R1262 nc-7 | R1262 ncRNA containing 25 bp | GGGAUAAUGAGCGUGUGACAAGAUUUUGGGCUUGUGUUUCGCA |
| (R1262_ncR | insertion in EMX1 site and MS2 | AGCUUUGAUUAAAAUGGUAGAUGGAUUUGCUAUCUAUCGUCAU |
| NA-EMX1- | stem loop at 3′ end | UUCCAGUACUGUUAUGUACAAACGGCAGAAGCUGGAGGAGGAA |
| MS2_25) | | GGGCCUGAGUCCGAGCAGAAGAAAACUCGAGCUCUGAAUGACUC |
| | | CUAUAAAGUUCUCCCAUCACAUCAACCGGUGGCGCAUUGCCACG |
| | | AAGCAGGCCAAUGGGGAGGACAUCGAUGUCAGCAUAAUGAGUU |
| | | GUCACACGCUCACAUGAGGAUCACCCAUGUGAU (SEQ ID NO: |
| | | 19191) |
| R1262_nc-6 | R1262 ncRNA containing P2A- | GGGAUAAUGAGCGUGUGACAAGAUUUUGGGCUUGUGUUUCGCA |
| (R1262_ncR | GFP insertion in EMX1 site | AGCUUUGAUUAAAAUGGUAGAUGGAUUUGCUAUCUAUCGUCAU |
| NA- | | UUCCAGUACUGUUAUGUACAAACGGCAGAAGCUGGAGGAGGAA |
| EMX1_P2A- | | GGGCCUGAGUCCGAGCAGAAGAAGGAAGCGGAGCCACUAACUUC |
| GFP) | | UCCCUGUUGAAACAAGCAGGGGAUGUCGAAGAGAAUCCCGGGCC |
| | | AGUGAGCAAGGGCGAGGAGCUGUUCACCGGGGUGGUGCCCAUCC |
| | | UGGUCGAGCUGGACGGCGACGUAAACGGCCACAAGUUCAGCGUG |
| | | UCCGGCGAGGGCGAGGGCGAUGCCACCUACGGCAAGCUGACCCU |
| | | GAAGUUCAUCUGCACCACCGGCAAGCUGCCCGUGCCCUGGCCCA |
| | | CCCUCGUGACCACCCUGACCUACGGCGUGCAGUGCUUCAGCCGC |
| | | UACCCCGACCACAUGAAGCAGCACGACUUCUUCAAGUCCGCCAU |
| | | GCCCGAAGGCUACGUCCAGGAGCGCACCAUCUUCUUCAAGGACG |
| | | ACGGCAACUACAAGACCCGCGCCGAGGUGAAGUUCGAGGGCGAC |
| | | ACCCUGGUGAACCGCAUCGAGCUGAAGGGCAUCGACUUCAAGGA |
| | | GGACGGCAACAUCCUGGGGCACAAGCUGGAGUACAACUACAACA |
| | | GCCACAACGUCUAUAUCAUGGCCGACAAGCAGAAGAACGGCAUC |
| | | AAGGUGAACUUCAAGAUCCGCCACAACAUCGAGGACGGCAGCGU |
| | | GCAGCUCGCCGACCACUACCAGCAGAACACCCCCAUCGGCGACG |
| | | GCCCCGUGCUGCUGCCCGACAACCACUACCUGAGCACCCAGUCC |
| | | GCCCUGAGCAAAGACCCCAACGAGAAGCGCGAUCACAUGGUCCU |
| | | GCUGGAGUUCGUGACCGCCGCCGGGAUCACUCUCGGCAUGGACG |
| | | AGCUGUACAAGUAGAAAGUUCUCCCAUCACAUCAACCGGUGGCG |
| | | CAUUGCCACGAAGCAGGCCAAUGGGGAGGACAUCGAUGUCAGCA |
| | | UAAUGAGUUGUCACACGCUCGAU (SEQ ID NO: 19192) |
| R1262_nc-22 | R1262 ncRNA containing 205 | GGGAUAAUGGGGCCACUAGGGACAGGAUGUUUCAGAGCUAUGC |
| (R1262_sgA | bp insertion in AAVSI site | UGGAAACAGCAUAGCAAGUUGAAAUAAGGCUAGUCCGUUAUCA |
| AVS1- | combined with sgAAVSI at 5′ | ACUUGAAAAAGUGGCACCGAGUCGGUGCUUUUUAAUAACAACG |
| ncRNA_205) | end | AGCGUGUGACAAGAUUUUGGGCUUGUGUUUCGCAAGCUUUGAU |
| | | UAAAAUGGUAGAUGGAUUUGCUAUCUAUCGUCAUUUCCAGUAC |
| | | UGUUAUGUUCUGUCCCCUCCACCCCACAGUGGGGCCACUAGGGA |
| | | CAGAAAGCGGUCUUACGGUCAGUCGUAUUCCUUCUCGAGUUCCG |
| | | UCCAGUUGAGCGUGUCACUCCCAGUGUACCUGCAAGCCGAGAUG |
| | | GCUGUGCUUGGAGUCAAUCGCAUGUAGGAUGGUCUCCAGACACC |
| | | GGGGCACCAGUUUUCACGCCUAAAGCAUAAACGACGAGCAGUCA |
| | | UGAAAGUCUUAGUACUGGACGUGCCGUUUCACAAUUAAUGACA |
| | | GAAAAGCCCCAUCCUUAGGCCUCCUCCUUCCUAGUCUCCUGAUA |
| | | UUGGGUCUAACCCCCAGCAUAAUGAGUUGUCACACGCUCGAU |
| | | (SEQ ID NO: 19193) |
| R1262_nc-8 | R1262 ncRNA containing 205 | GGGAUAAUGAGCGUGUGACAAGAUUUUGGGCUUGUGUUUCGCA |
| (R1262_ncR | bp insertion in EMX1 site and | AGCUUUGAUUAAAAUGGUAGAUGGAUUUGCUAUCUAUCGUCAU |
| NA-EMX1- | MS2 stem loop at 3′ end | UUCCAGUACUGUUAUGUACAAACGGCAGAAGCUGGAGGAGGAA |
| MS2_205) | | GGGCCUGAGUCCGAGCAGAAGAAAAAGCGGUCUUACGGUCAGUC |
| | | GUAUUCCUUCUCGAGUUCCGUCCAGUUGAGCGUGUCACUCCCAG |
| | | UGUACCUGCAAGCCGAGAUGGCUGUGCUUGGAGUCAAUCGCAUG |
| | | UAGGAUGGUCUCCAGACACCGGGGCACCAGUUUUCACGCCUAAA |
| | | GCAUAAACGACGAGCAGUCAUGAAAGUCUUAGUACUGGACGUGC |
| | | CGUUUCACAAAGUUCUCCCAUCACAUCAACCGGUGGCGCAUUGC |
| | | CACGAAGCAGGCCAAUGGGGAGGACAUCGAUGUCAGCAUAAUGA |
| | | GUUGUCACACGCUCACAUGAGGAUCACCCAUGUGAU (SEQ ID NO: |
| | | 19194) |
| R1262_nc-21 | R1262 ncRNA containing 205 | GGGAUAAUGAGCGUGUGACAAGAUUUUGGGCUUGUGUUUCGCA |
| (R1262_ncR | bp insertion in AAVSI site | AGCUUUGAUUAAAAUGGUAGAUGGAUUUGCUAUCUAUCGUCAU |
| NA- | combined with sgAAVSI at 3′ | UUCCAGUACUGUUAUGUUCUGUCCCCUCCACCCCACAGUGGGGC |
| sgAAVS1_205) | end | CACUAGGGACAGAAAGCGGUCUUACGGUCAGUCGUAUUCCUUCU |
| | | CGAGUUCCGUCCAGUUGAGCGUGUCACUCCCAGUGUACCUGCAA |
| | | GCCGAGAUGGCUGUGCUUGGAGUCAAUCGCAUGUAGGAUGGUC |
| | | UCCAGACACCGGGGCACCAGUUUUCACGCCUAAAGCAUAAACGA |
| | | CGAGCAGUCAUGAAAGUCUUAGUACUGGACGUGCCGUUUCACAA |
| | | UUAAUGACAGAAAAGCCCCAUCCUUAGGCCUCCUCCUUCCUAGU |
| | | CUCCUGAUAUUGGGUCUAACCCCCAGCAUAAUGAGUUGUCACAC |
| | | GCUCAAUAACAACGGGGCCACUAGGGACAGGAUGUUUCAGAGCU |
| | | AUGCUGGAAACAGCAUAGCAAGUUGAAAUAAGGCUAGUCCGUU |
| | | AUCAACUUGAAAAAGUGGCACCGAGUCGGUGCUUUUUGAU (SEQ |
| | | ID NO: 19195) |
| R1262_nc-10 | R1262 ncRNA containing 405 | GGGAUAAUGAGCGUGUGACAAGAUUUUGGGCUUGUGUUUCGCA |
| (R1262_ncR | bp insertion in EMX1 site and | AGCUUUGAUUAAAAUGGUAGAUGGAUUUGCUAUCUAUCGUCAU |
| NA-EMX1- | MS2 stem loop at 3′ end | UUCCAGUACUGUUAUGUACAAACGGCAGAAGCUGGAGGAGGAA |
| MS2_405) | | GGGCCUGAGUCCGAGCAGAAGAACUGCUAAAUCCGCGUGAUAGG |
| | | GGAUUUGAAGUUUAAUCUUCUAUCGCAAGGAACUGCCGAUCUU |
| | | AAUGGAUGGCCGGAGGUGGUAUGGAAGCUAUAAGCGCGGGUGA |
| | | GAGGGUAAUUAGGCGUGUUCACCUACGCUACGCUAACGGGCGAU |
| | | UCUAUAAGAUUGCACAUUGCGUCAACUCAUAAGAUGUCUCAACG |
| | | GCAUGCGCAACUUGUGAAGUGUCUACUAUCCUUAAACGCAUAUC |
| | | UCGCACAGUAACUCCCGAAUAUGUCGGCAUCUGAUGUUGCCCGG |
| | | GCCGAGUUAGUGUUGAGCUCACGGAACUUAUUGUAUGAGUAGU |
| | | GAUUUGUAAGAGUUGUCAGUUAGCUCGUUCAGGUAAUAGUUGC |
| | | CCACACAACGUCAAAAUAAGAGAACGGUCGUAACAUAAAGUUCU |
| | | CCCAUCACAUCAACCGGUGGCGCAUUGCCACGAAGCAGGCCAAU |
| | | GGGGAGGACAUCGAUGUCAGCAUAAUGAGUUGUCACACGCUCAC |
| | | AUGAGGAUCACCCAUGUGAU (SEQ ID NO: 19196) |
| R1262_nc-11 | R1262 ncRNA containing 505 | GGGAUAAUGAGCGUGUGACAAGAUUUUGGGCUUGUGUUUCGCA |
| (R1262_ncR | bp insertion in EMX1 site and | AGCUUUGAUUAAAAUGGUAGAUGGAUUUGCUAUCUAUCGUCAU |
| NA-EMX1- | MS2 stem loop at 3′ end | UUCCAGUACUGUUAUGUACAAACGGCAGAAGCUGGAGGAGGAA |
| MS2_505) | | GGGCCUGAGUCCGAGCAGAAGAAACUUCAGCUAAGGAAGCUACC |
| | | AAUAUUUAGUUUCUGAGUCUCACGACAGACCUCGCGCGUAGAUU |
| | | GCCAUGCGUAGAGCUAACGAGCCAGCGGAAAGCGUGAGGCGCUU |
| | | UUAAGCAUGGCGAGUAAGUGAUCCAACGCUUCGGAUAUGACUA |
| | | UAUACUUAGGUUCGAUCUCGUCCCGAGAAUUCUAAGCCUCAACA |
| | | UCUAUGAGUUAUGAGGUUAGCCGAAAAAGCACGUGGUGGCGCCC |
| | | ACCGACUGUUCCCAGACUGUAGCUCUUUGUUCUGUCAAGGCCCG |
| | | ACCUUCAUCGCGGCCGAUUCCUUCUGCGGACCAUACCGUCCUGA |
| | | UACUUUGGUCAUGUUUCCGUUGUAGGAGUGAACCCACUUGCCUU |
| | | UGCGUCUUAAUACCAAUGAAAAACCUAUGCACUUUGUACAGGGU |
| | | ACCAUCGGGAUUCUGAACCCUCAGAUAGUGGGGAUCCCGGGUAU |
| | | AGACCUUUAUCUGCGGUCCAACUUAGGCAUAAACCUCCAUGCUA |
| | | CAAAGUUCUCCCAUCACAUCAACCGGUGGCGCAUUGCCACGAAG |
| | | CAGGCCAAUGGGGAGGACAUCGAUGUCAGCAUAAUGAGUUGUC |
| | | ACACGCUCACAUGAGGAUCACCCAUGUGAU (SEQ ID NO: 19197) |
| R1262_nc-12 | R1262 ncRNA containing P2A- | GGGAUAAUGAGCGUGUGACAAGAUUUUGGGCUUGUGUUUCGCA |
| (R1262_ncR | GFP insertion in EMX1 site and | AGCUUUGAUUAAAAUGGUAGAUGGAUUUGCUAUCUAUCGUCAU |
| NA-EMX1- | MS2 stem loop at 3′ end | UUCCAGUACUGUUAUGUACAAACGGCAGAAGCUGGAGGAGGAA |
| MS2_P2A- | | GGGCCUGAGUCCGAGCAGAAGAAGGAAGCGGAGCCACUAACUUC |
| GFP) | | UCCCUGUUGAAACAAGCAGGGGAUGUCGAAGAGAAUCCCGGGCC |
| | | AGUGAGCAAGGGCGAGGAGCUGUUCACCGGGGUGGUGCCCAUCC |
| | | UGGUCGAGCUGGACGGCGACGUAAACGGCCACAAGUUCAGCGUG |
| | | UCCGGCGAGGGCGAGGGCGAUGCCACCUACGGCAAGCUGACCCU |
| | | GAAGUUCAUCUGCACCACCGGCAAGCUGCCCGUGCCCUGGCCCA |
| | | CCCUCGUGACCACCCUGACCUACGGCGUGCAGUGCUUCAGCCGC |
| | | UACCCCGACCACAUGAAGCAGCACGACUUCUUCAAGUCCGCCAU |
| | | GCCCGAAGGCUACGUCCAGGAGCGCACCAUCUUCUUCAAGGACG |
| | | ACGGCAACUACAAGACCCGCGCCGAGGUGAAGUUCGAGGGCGAC |
| | | ACCCUGGUGAACCGCAUCGAGCUGAAGGGCAUCGACUUCAAGGA |
| | | GGACGGCAACAUCCUGGGGCACAAGCUGGAGUACAACUACAACA |
| | | GCCACAACGUCUAUAUCAUGGCCGACAAGCAGAAGAACGGCAUC |
| | | AAGGUGAACUUCAAGAUCCGCCACAACAUCGAGGACGGCAGCGU |
| | | GCAGCUCGCCGACCACUACCAGCAGAACACCCCCAUCGGCGACG |
| | | GCCCCGUGCUGCUGCCCGACAACCACUACCUGAGCACCCAGUCC |
| | | GCCCUGAGCAAAGACCCCAACGAGAAGCGCGAUCACAUGGUCCU |
| | | GCUGGAGUUCGUGACCGCCGCCGGGAUCACUCUCGGCAUGGACG |
| | | AGCUGUACAAGUAGAAAGUUCUCCCAUCACAUCAACCGGUGGCG |
| | | CAUUGCCACGAAGCAGGCCAAUGGGGAGGACAUCGAUGUCAGCA |
| | | UAAUGAGUUGUCACACGCUCACAUGAGGAUCACCCAUGUGAU |
| | | (SEQ ID NO: 19198) |
| R1262_nc-23 | Same as R1262_nc-6 but with | GGGAUAAUGAGCGUGUGACAAGAUUUUGGGCUUGUGUUUCGCA |
| (R1262_ncR | longer homology arms | AGCUUUGAUUAAAAUGGUAGAUGGAUUUGCUAUCUAUCGUCAU |
| NA- | | UUCCAGUACUGUUAUGUGAGGCCCCAGUGGCUGCUCUGGGGGCC |
| EMX1 P2A- | | UCCUGAGUUUCUCAUCUGUGCCCCUCCCUCCCUGGCCCAGGUGA |
| GFP_LongIIA) | | AGGUGUGGUUCCAGAACCGGAGGACAAAGUACAAACGGCAGAA |
| | | GCUGGAGGAGGAAGGGCCUGAGUCCGAGCAGAAGAAGGAAGCG |
| | | GAGCCACUAACUUCUCCCUGUUGAAACAAGCAGGGGAUGUCGAA |
| | | GAGAAUCCCGGGCCAGUGAGCAAGGGCGAGGAGCUGUUCACCGG |
| | | GGUGGUGCCCAUCCUGGUCGAGCUGGACGGCGACGUAAACGGCC |
| | | ACAAGUUCAGCGUGUCCGGCGAGGGCGAGGGCGAUGCCACCUAC |
| | | GGCAAGCUGACCCUGAAGUUCAUCUGCACCACCGGCAAGCUGCC |
| | | CGUGCCCUGGCCCACCCUCGUGACCACCCUGACCUACGGCGUGC |
| | | AGUGCUUCAGCCGCUACCCCGACCACAUGAAGCAGCACGACUUC |
| | | UUCAAGUCCGCCAUGCCCGAAGGCUACGUCCAGGAGCGCACCAU |
| | | CUUCUUCAAGGACGACGGCAACUACAAGACCCGCGCCGAGGUGA |
| | | AGUUCGAGGGCGACACCCUGGUGAACCGCAUCGAGCUGAAGGGC |
| | | AUCGACUUCAAGGAGGACGGCAACAUCCUGGGGCACAAGCUGGA |
| | | GUACAACUACAACAGCCACAACGUCUAUAUCAUGGCCGACAAGC |
| | | AGAAGAACGGCAUCAAGGUGAACUUCAAGAUCCGCCACAACAUC |
| | | GAGGACGGCAGCGUGCAGCUCGCCGACCACUACCAGCAGAACAC |
| | | CCCCAUCGGCGACGGCCCCGUGCUGCUGCCCGACAACCACUACCU |
| | | GAGCACCCAGUCCGCCCUGAGCAAAGACCCCAACGAGAAGCGCG |
| | | AUCACAUGGUCCUGCUGGAGUUCGUGACCGCCGCCGGGAUCACU |
| | | CUCGGCAUGGACGAGCUGUACAAGUAGAAAGUUCUCCCAUCACA |
| | | UCAACCGGUGGCGCAUUGCCACGAAGCAGGCCAAUGGGGAGGAC |
| | | AUCGAUGUCACCUCCAAUGACUAGGGUGGGCAACCACAAACCCA |
| | | CGAGGGCAGAGUGCUGCUUGCUGCUGGCCAGGCCCCUGCGUGGG |
| | | CCCAAGCGCAUAAUGAGUUGUCACACGCUCGAU (SEQ ID NO: |
| | | 19199) |
| R1262_nc-24 | Same as R1262_nc-12 but with | GGGAUAAUGAGCGUGUGACAAGAUUUUGGGCUUGUGUUUCGCA |
| (R1262_ncR | longer homology arms | AGCUUUGAUUAAAAUGGUAGAUGGAUUUGCUAUCUAUCGUCAU |
| NA-EMX1- | | UUCCAGUACUGUUAUGUGAGGCCCCAGUGGCUGCUCUGGGGGCC |
| MS2 P2A- | | UCCUGAGUUUCUCAUCUGUGCCCCUCCCUCCCUGGCCCAGGUGA |
| GFP_LongHA) | | AGGUGUGGUUCCAGAACCGGAGGACAAAGUACAAACGGCAGAA |
| | | GCUGGAGGAGGAAGGGCCUGAGUCCGAGCAGAAGAAGGAAGCG |
| | | GAGCCACUAACUUCUCCCUGUUGAAACAAGCAGGGGAUGUCGAA |
| | | GAGAAUCCCGGGCCAGUGAGCAAGGGCGAGGAGCUGUUCACCGG |
| | | GGUGGUGCCCAUCCUGGUCGAGCUGGACGGCGACGUAAACGGCC |
| | | ACAAGUUCAGCGUGUCCGGCGAGGGCGAGGGCGAUGCCACCUAC |
| | | GGCAAGCUGACCCUGAAGUUCAUCUGCACCACCGGCAAGCUGCC |
| | | CGUGCCCUGGCCCACCCUCGUGACCACCCUGACCUACGGCGUGC |
| | | AGUGCUUCAGCCGCUACCCCGACCACAUGAAGCAGCACGACUUC |
| | | UUCAAGUCCGCCAUGCCCGAAGGCUACGUCCAGGAGCGCACCAU |
| | | CUUCUUCAAGGACGACGGCAACUACAAGACCCGCGCCGAGGUGA |
| | | AGUUCGAGGGCGACACCCUGGUGAACCGCAUCGAGCUGAAGGGC |
| | | AUCGACUUCAAGGAGGACGGCAACAUCCUGGGGCACAAGCUGGA |
| | | GUACAACUACAACAGCCACAACGUCUAUAUCAUGGCCGACAAGC |
| | | AGAAGAACGGCAUCAAGGUGAACUUCAAGAUCCGCCACAACAUC |
| | | GAGGACGGCAGCGUGCAGCUCGCCGACCACUACCAGCAGAACAC |
| | | CCCCAUCGGCGACGGCCCCGUGCUGCUGCCCGACAACCACUACCU |
| | | GAGCACCCAGUCCGCCCUGAGCAAAGACCCCAACGAGAAGCGCG |
| | | AUCACAUGGUCCUGCUGGAGUUCGUGACCGCCGCCGGGAUCACU |
| | | CUCGGCAUGGACGAGCUGUACAAGUAGAAAGUUCUCCCAUCACA |
| | | UCAACCGGUGGCGCAUUGCCACGAAGCAGGCCAAUGGGGAGGAC |
| | | AUCGAUGUCACCUCCAAUGACUAGGGUGGGCAACCACAAACCCA |
| | | CGAGGGCAGAGUGCUGCUUGCUGCUGGCCAGGCCCCUGCGUGGG |
| | | CCCAAGCGCAUAAUGAGUUGUCACACGCUCACAUGAGGAUCACC |
| | | CAUGUGAU (SEQ ID NO: 19200) |
| 6083 v1 RT | 6083 retron RT mRNA | AUGAGCAACCCUCAGCCCACCAGGGCUGAGAUCUUCGAGCGCAU |
| | | CAAACAGAGCUCGAAGCAGGAGGUGAUCCUGGAGGAGAUGCAG |
| | | AGACUGGGCUUCUGGCCAAGGUCAGAAGGCCAGCCUGAGGUCGC |
| | | UGCCGACCUCAUCCAGAGAGAAGGCGAGCUCCAAAGGGAGCUCG |
| | | CCGAACUCAACAAGAAGUUGGCGGUCAAACGCAACCCCGAGAGG |
| | | GCCCUCAGAGAAAUGAGGAAGCAGAGAAUGAAGGACGCCAGAG |
| | | ACAAGAGAGAGGUGACCAAGAGAGCCCAGGCUCAGCAGAGAUAC |
| | | GACAAGGCCCUGCUGUGGCACGAGAAGAGGGCCAGCCACGUGGC |
| | | CUAUCUUGGCCCUGGUGUGAGCGCCAGCUUACACGAGAACAGCU |
| | | CUGCUACCCAGGAGCAGGGCGACAAGGGGAAACCCAAGAGAGCC |
| | | CGAGAUCGGGCUGUGCCAGACCUGCAGAGGCUGACACUGAACGG |
| | | GCUGCCUGCUCUGAUUAGCGCAGCCCAACUCGCUGAGUCCAUGG |
| | | GAGUCAGCGUGGCUGAGCUGAGAUUCCUCAGCUUCCACAGGGAG |
| | | GUGGCUAGGACAAACCACUACCACAGCUUCACGCUCCCCAAGAA |
| | | GACAGGUGGGGAGCGUCUGAUCAGCGCCCCCAUGCCCAGACUGA |
| | | AGAGAGCCCAGUACUGGGUGCUGGACAACGUGCUGGCAAAGAUG |
| | | CCAGCGCACGAUGCGGCUCACGGCUUCCUGGCCGGCAGAAGCAU |
| | | CAUCAGCAAUGCCAAGCCCCAUGCGGGGCAGGAUGUGGUCAUUA |
| | | ACUUGGACGUUAAGGACUUCUUCCCCAGCAUCGCCUUCGGCAGA |
| | | AUCAAGGGCGUGUUCAGACAGCUGGGCUACGGCGAAAGCAUCGC |
| | | CACAGUGUUCGCCCUGCUGUGCAGCGAGAACAGAGCCCAGGCCU |
| | | GGCAAGUGGACGGAGAGAGACUCUUCGUCGGCGGCAAGGCCAGA |
| | | GAAAGAGUGCUGCCUCAGGGCGCUCCUACCAGCCCCAUGCUGAC |
| | | CAACCUGCUGUGCCGGCGGAUGGAUAGACGUCUGCUGGGUCUCG |
| | | CGAAGCAGCUGGGCUUCGUGUACACCAGAUACGCCGACGACCUG |
| | | ACCUUCAGCGCCUCCGGCGAACCCGCAAGGGAUAACGUCGGAAA |
| | | GCUGCUGAGCAGGGUCCGGUGGAUCCUGAGGGAUGAGGGGUUC |
| | | ACCCCUCAUCCCGAUAAGGAGAGAGUGAUGAGAAAGGGCAGAAG |
| | | ACAGGAGGUGACCGGCCUUGUGGUCAACUCCGACACUCCCAGCG |
| | | UGAGCAGGGAGACCAGAAGAAGGCUGAGAGCCGCUCUGCACAGA |
| | | GCCUCGCAGCCUGACGCUGCGAGCAAACCUGCACAUUGGCAGGG |
| | | CCAUACGGCCCAGCCAAGUCAGCUGCUGGGCCUGGCCACAUUCG |
| | | UGCAUCAGAUCGACCCCAAGCAGGGCAAGACCCUGCUCGCUGAC |
| | | GCUCAGCAACUGAUGCGCAGCCCUAUCGACCGCGCUAAUGACGC |
| | | GGCGAAGUCUGCUAGCAGGGCUGACGCUGCUCAGCAGAGCUUCA |
| | | GAGUGCUGGCUGCUGCCGGCAAGCCACCAGUUCUUGCCGACGGC |
| | | AAGAACUGGUGGCAACCUGCUCCUCCUGCCACACCUGUGCUGGA |
| | | GAAGACCGACCAGCAGAGAAGAGAGGAAAGGCAGGCUACCAGAA |
| | | GGCAGCAGGCCGCUGCUGCUGCUCCUCCUCCUAGCAGCACAAGA |
| | | AGAAACGAGCGGCCGCAACAGGCCGCUCAUGAGCAGCAGGGAGA |
| | | UGCCCAGCCUCAGAACGAGGCCCCUCCUAGAUUCGACCCCGACC |
| | | AGUACGCUCCUCCCCCGAGGAACGUGAUGACCUACUGGGCCCAG |
| | | AUCGCCAUCAGCUUCUUUCUGGGCAGCAUCCUGCACAACAGACU |
| | | GAUCACGAUCUUCGCCAUGGUGGCGGUGAUCGCUCUGUACUACA |
| | | UGAGAAGACAGAGAUGGGAUGUCUUCAUGGGCAUCCUGGUGGU |
| | | GGCCACCCUGCUGGGAUACCUGGUCAGGGGCAUGGGCccaccaaagaa |
| | | gaaaagaaaggucuga (SEQ ID NO: 19201) |
| 6083 | R6083 retron containing 25 bp | GCUCCGGAGCAAUGAGCAGGCUCUUGCAAUCCGGGCGGUGUUUC |
| vl_ncRNA_A | insertion at AAVSI site | GCCGCCCUUGUGAACUGCCGUUUCAUGCACCACGGGCGCCGUUU |
| AVS1 | | UCACUGUGCCACCCCAGCCACGGUAGUGCUUCUGUCCCCUCCAC |
| gRNA_25bp | | CCCACAGUGGGGCCACUAGGGACAGAAAUGACAGUGGUUGGUGC |
| | | UCUAAAAAUUAAUGACAGAAAAGCCCCAUCCUUAGGCCUCCUCC |
| | | UUCCUAGUCUCCUGAUAUUGGGUCUAACCCCCAAGCACUACCGU |
| | | GGCGGGGUGGCGUCGAGCGAACAGCUCCCGUCCCCUGAGCCCUA |
| | | CAGGCUCUUGGACGAGAUGCACAUUGCUCCGGAGCAAUAACAAC |
| | | GGGGCCACUAGGGACAGGAUguuucagagcuaugcuggaaacag |
| | | cauagcaaguugaaauaaggcuaguccguuaucaacuugaaaaa |
| | | guggcaccgagucggugcuuuuu (SEQ ID NO: 19202) |
| 6083 | R6083 retron containing 25 bp | GCUCCGGAGCAAUGAGCAGGCUCUUGCAAUCCGGGCGGUGUUUC |
| vl_ncRNA | insertion at EMX1 site | GCCGCCCUUGUGAACUGCCGUUUCAUGCACCACGGGCGCCGUUU |
| only_25bp | | UCACUGUGCCACCCCAGCCACGGUAGUGCUacaaacggcagaagcuggagga |
| | | ggaagggccugaguccgagcagaagaaaACUCGAGCUCUGAAUGACUCCUAUAa |
| | | aguucucccaucacaucaaccgguggcgcauugccacgaagcaggccaaugggg |
| | | aggacaucgaugucaAGCACUACCGUGGCGGGGUGGCGUCGAGCGAACAGCUCC |
| | | CGUCCCCUGAGCCCUACAGGCUCUUGGACGAGAUGCACAUUGCUCCG |
| | | GAGC (SEQ ID NO: 19203) |
| 6083 | R6083 retron containing 25 bp | GCUCCGGAGCAAUGAGCAGGCUCUUGCAAUCCGGGCGGUGUUUC |
| vl_ncRNA_E | insertion at EMX1 site and | GCCGCCCUUGUGAACUGCCGUUUCAUGCACCACGGGCGCCGUUU |
| MX1 | sgEMX1 at 3′ end | UCACUGUGCCACCCCAGCCACGGUAGUGCUacaaacggcagaagcuggagga |
| gRNA_25bp | | ggaagggccugaguccgagcagaagaaaACUCGAGCUCUGAAUGACUCCUAUAa |
| | | aguucucccaucacaucaaccgguggcgcauugccacgaagcaggccaaugggg |
| | | aggacaucgaugucaAGCACUACCGUGGCGGGGUGGCGUCGAGCGAACAGCUCC |
| | | CGUCCCCUGAGCCCUACAGGCUCUUGGACGAGAUGCACAUUGCUCCG |
| | | GAGCAAUAACAACgaguccgagcagaagaagaaguuucagagcuaugcuggaaa |
| | | cagca(SEQ ID NO: 19204) |
| 6083 | R6083 retron containing GFP | GCUCCGGAGCAAUGAGCAGGCUCUUGCAAUCCGGGCGGUGUUUC |
| vl_ncRNA_E | gene insertion at EMX1 site and | GCCGCCCUUGUGAACUGCCGUUUCAUGCACCACGGGCGCCGUUU |
| MX1 | sgEMX1 at 3′ end | UCACUGUGCCACCCCAGCCACGGUAGUGCUacaaacggcagaagcuggagga |
| gRNA_GFP | | ggaagggccugaguccgagcagaagaacccauauguccuuccgagugagaga |
| gene | | cacaaaaaauuccaacacacuauugcaaugaaaauaaauuuccuuuauuagc |
| | | cagaagucagaugcucaaggggcuucaugauguccccauaauuuuuggcaga |
| | | gggaaaaagaucucagugguauuugugagccagggcauuggccacaccagccac |
| | | caccuucugauaggcagccugcaccugaggagugcggccgcuuuacuuguacag |
| | | cucguccaugccgagagugaucccggggggucacgaacuccagcaggaccaugu |
| | | gaucgcgcuucucguuggggucuuugcucagggggacugggugcucagguagug |
| | | guugucgggcagcagcacggggccgucgccgauggggguguucugugguagggg |
| | | ggggcacgugcuccugauuugccgucguccuugaagaagauggugcgcuccugg |
| | | acguagccuucgggcauggcggacuugaagaagucgugcugcuucauguggucg |
| | | ggguagcggcugaagcacugcacgccguaggucaggguggucacgagggugggc |
| | | cagggcacgggcagcuugccgguggugcagaugaacuucagggucagcuugagc |
| | | ucgaccaggaugggcaccaccccggugaacagcuccucgcccuugcucaccaug |
| | | guggcgaccgguggaucccgggcccgcgguaccgucgacugcagaauucgaagc |
| | | uugagcucgagaucugaguccgguagcgcuagcggaucugacgguucacuaaac |
| | | ccuguguucuggggcaaacccguugcgaaaaagaacguucacggcgacuacugc |
| | | acuuauauacgguucucccccacccucgggaaaaaggcggagccaguacacgac |
| | | aucacuuucccaguuuaccccgcgccaccuucucuaggcaccgguucaauugcc |
| | | gaccccuccccccaacuucucggggacuguggggauguggcucugccguucucc |
| | | caucacaucaaccgguggcgcauugccacgaagcaggccaauggggaggacauc |
| | | gaugucaAGCACUACCGUGGCGGGGUGGCGUCGAGCGAACAGCUCCCGUCCCCU |
| | | GAGCCCUACAGGCUCUUGGACGAGAUGCACAUUGCUCCGGAGCAAUAACAAC |
| | | gaguccgagcagaagaagaaguuucagagcuaugcuggaaacagcauagcaa |
| | | guugaaauaaggcuaguccguuaucaacuugaaaaaguggcaccgagucggu |
| | | guuuuu (SEQ ID NO: 19205) |
| 6083 | R6083 retron containing 50 bp | GCUCCGGAGCAAUGAGCAGGCUCUUGCAAUCCGGGCGGUGUUUCGCCGCC |
| vl_ncRNA_E | insertion at EMX1 site and | CUUGUGAACUGCCGUUUCAUGCACCACGGGCGCCGUUUUCACUGUGCCAC |
| MX1 | sgEMX1 at 3′ end | CCCAGCCACGGUAGUGCUacaaacggcagaagcuggaggaggaagggccu |
| gRNA_50bp | | gaguccgagcagaagaaaACUCGAGCUCUGAUCAGUAUCAUGGGGCCCAU |
| | | GGUUGAAUGACUCCUAUAaaguucucccaucacaucaaccgguggcgcauu |
| | | gccacgaagcaggccaauggggaggacaucgaugucaAGCACUACCGUGG |
| | | CGGGGUGGCGUCGAGCGAACAGCUCCCGUCCCCUGAGCCCUACAGGCU |
| | | CUUGGACGAGAUGCACAUUGCUCCGGAGCAAUAACAACgaguccgagcag |
| | | aagaagaaguuucagagcuaugcuggaaacagcauagcaaguugaaauaa |
| | | ggcuaguccguuaucaacuugaaaaaguggcaccgagucggugcuuuuu |
| | | (SEQ ID NO: 19206) |
| 6083 | R6083 retron containing 100 bp | GCUCCGGAGCAAUGAGCAGGCUCUUGCAAUCCGGGCGGUGUUUC |
| vl_ncRNA_E | insertion at EMX1 site and | GCCGCCCUUGUGAACUGCCGUUUCAUGCACCACGGGCGCCGUUU |
| MX1 | sgEMX1 at 3′ end | UCACUGUGCCACCCCAGCCACGGUAGUGCUacaaacggcagaagcuggagga |
| gRNA_100bp | | ggaagggccugaguccgagcagaagaaaACUCGAGCUCUGAGCCCCACUGUCG |
| | | AGAAGUAUGUAUCUCGCUCCCGCAGCUUGCCAGCACUUUCAGUAUCAUGGG |
| | | GCCCAUGGUUGAAUGACUCCUAUAaaguucucccaucacaucaaccgguggcg |
| | | cauugccacgaagcaggccaauggggaggacaucgaugucaAGCACUACCG |
| | | UGGCGGGGUGGCGUCGAGCGAACAGCUCCCGUCCCCUGAGCCCU |
| | | ACAGGCUCUUGGACGAGAUGCACAUUGCUCCGGAGCAAUAACAA |
| | | Cgaguccgagcagaagaagaaguuucagagcuaugcuggaaaca |
| | | gcauagcaaguugaaauaaggcuaguccguuaucaacuugaaaa |
| | | aguggcaccgagucggugcuuuuu (SEQ ID NO: 19207) |
| 6083 | R6083 rctron containing 75 bp | GCUCCGGAGCAAUGAGCAGGCUCUUGCAAUCCGGGCGGUGUUUC |
| vl_ncRNA_E | insertion at EMX1 site and | GCCGCCCUUGUGAACUGCCGUUUCAUGCACCACGGGCGCCGUUU |
| MX1 | sgEMX1 at 3′ end | UCACUGUGCCACCCCAGCCACGGUAGUGCUacaaacggcagaagcuggagga |
| gRNA_75bp | | ggaagggccugaguccgagcagaagaaaACUCGAGCUCUGAGCCCCACCGCAGC |
| | | UUGCCAGCACUUUCAGUAUCAUGGGGCCCAUGGUUGAAUGACUCCUAUAaagu |
| | | ucucccaucacaucaaccgguggcgcauugccacgaagcaggccaauggggagga |
| | | caucgaugucaAGCACUACCGUGGCGGGGUGGCGUCGAGCGAACAGC |
| | | UCCCGUCCCCUGAGCCCUACAGGCUCUUGGACGAGAUGCACAUU |
| | | GCUCCGGAGCAAUAACAACgaguccgagcagaagaagaaguuucagagcuaugcu |
| | | ggaaacagcauagcaaguugaaauaaggcuaguccguuaucaacuugaaaaagug |
| | | gcaccgagucggugcuuuuu (SEQ ID NO: 19208) |
| Aco1_ncRNA- | Aco1 ncRNA containing 100 bp | GGGAUAAUGUAUAAAACCGGGAACGAUCAGACCGGGGUGAAUU |
| EMX1_100 | insertion in EMX1 gene | CGCCCCCUUGAUCAAACGGCACUAACCACUGUUUGCCGUGCGUG |
| | | CGUACAAACGGCAGAAGCUGGAGGAGGAAGGGCCUGAGUCCGAG |
| | | CAGAAGAAAACUCGAGCUCUGAGCCCCACUGUCGAGAAGUAUGU |
| | | AUCUCGCUCCCGCAGCUUGCCAGCACUUUCAGUAUCAUGGGGCC |
| | | CAUGGUUGAAUGACUCCUAUAAAGUUCUCCCAUCACAUCAACCG |
| | | GUGGCGCAUUGCCACGAAGCAGGCCAAUGGGGAGGACAUCGAUG |
| | | UCAACGCACGCACGGCAAACAGACAGAUCCAUUAUUAUUACAAU |
| | | UUAUUUAGUGAUCGUUCCCGGUUUUAUACGAU (SEQ ID NO: |
| | | 19209) |
| Aco1_ncRNA- | Aco1 ncRNA containing 205 bp | GGGAUAAUGUAUAAAACCGGGAACGAUCAGACCGGGGUGAAUU |
| EMX1_205 | insertion in EMX1 gene | CGCCCCCUUGAUCAAACGGCACUAACCACUGUUUGCCGUGCGUG |
| | | CGUACAAACGGCAGAAGCUGGAGGAGGAAGGGCCUGAGUCCGAG |
| | | CAGAAGAAAAAGCGGUCUUACGGUCAGUCGUAUUCCUUCUCGAG |
| | | UUCCGUCCAGUUGAGCGUGUCACUCCCAGUGUACCUGCAAGCCG |
| | | AGAUGGCUGUGCUUGGAGUCAAUCGCAUGUAGGAUGGUCUCCA |
| | | GACACCGGGGCACCAGUUUUCACGCCUAAAGCAUAAACGACGAG |
| | | CAGUCAUGAAAGUCUUAGUACUGGACGUGCCGUUUCACAAAGUU |
| | | CUCCCAUCACAUCAACCGGUGGCGCAUUGCCACGAAGCAGGCCA |
| | | AUGGGGAGGACAUCGAUGUCAACGCACGCACGGCAAACAGACAG |
| | | AUCCAUUAUUAUUACAAUUUAUUUAGUGAUCGUUCCCGGUUUU |
| | | AUACGAU (SEQ ID NO: 19210) |
| Eco3_ncRNA- | Eco3 ncRNA containing 205 bp | GGGAUAAUUGAUAAGAUUCCGUAAGAGCCAAACCUAGCAUUUU |
| EMX1 205 | insertion in EMX1 gene | AUGGGUUAAUAGCCCAUCGGGCCAUGAGUCAUGGUUUCGCCUAG |
| | | UAUUUUAGCUAUGCCCGUCGUUCAGUUCGCUGAACAAACGGCAG |
| | | AAGCUGGAGGAGGAAGGGCCUGAGUCCGAGCAGAAGAAAAAGC |
| | | GGUCUUACGGUCAGUCGUAUUCCUUCUCGAGUUCCGUCCAGUUG |
| | | AGCGUGUCACUCCCAGUGUACCUGCAAGCCGAGAUGGCUGUGCU |
| | | UGGAGUCAAUCGCAUGUAGGAUGGUCUCCAGACACCGGGGCACC |
| | | AGUUUUCACGCCUAAAGCAUAAACGACGAGCAGUCAUGAAAGUC |
| | | UUAGUACUGGACGUGCCGUUUCACAAAGUUCUCCCAUCACAUCA |
| | | ACCGGUGGCGCAUUGCCACGAAGCAGGCCAAUGGGGAGGACAUC |
| | | GAUGUCAUCAGCGAACUGAUCGACGUGCUCAAGUAGGUUUGGCU |
| | | CUUACGGAAUCUUAUCAGAU (SEQ ID NO: 19211) |

### Primer Sequences

| Description | Sequence |
|---|---|
| AAVSI_NGS_F | TCGTCGGCAGCGTCAGATGTGTATAAGAGACAGAGCTG |
| | GGACCACCTTATATTCC(SEQ ID NO: 19212) |
| AAVS1_NGS_R | GTCTCGTGGGCTCGGAGATGTGTATAAGAGACAGGTTC |
| | TGGCAAGGAGAGAGATG(SEQ ID NO: 19213) |
| EMX1_NGS_F3 | TCGTCGGCAGCGTCAGATGTGTATAAGAGACAGaggtg |
| | aaggtgtggttccag(SEQ ID NO: 19214) |
| EMX1_NGS_R3 | GTCTCGTGGGCTCGGAGATGTGTATAAGAGACAGtagt |
| | cattggaggtgacatcg(SEQ ID NO: 19215) |
| EMX1_NGS_F1 | TCGTCGGCAGCGTCAGATGTGTATAAGAGACAGaggtg |
| | aaggtgtggttccag(SEQ ID NO: 19216) |
| EMX1_NGS_R1 | GTCTCGTGGGCTCGGAGATGTGTATAAGAGACAGgcca |
| | gagtccagcttggg(SEQ ID NO: 19217) |

qPCR Primers

| qPCR Primers- NOT NGS | |
|---|---|
| EMX1 Forward Primer | aggtgaaggtgtggttccag |
| | (SEQ ID NO: 19218) |
| EMX1 Reverse Primer | tagtcattggaggtgacatcg |
| | (SEQ ID NO: 19219) |
| 5′ Junction GFP | ccccttgagcatctgacttc |
| Reverse Primer | (SEQ ID NO: 19220) |
| 3′ Junction GFP | catcactttcccagtttaccc |
| Forward Primer | (SEQ ID NO: 19221) |

### RT Variants (Coding Sequences)

| RT Variant | ORF Sequence | SEQ ID NO: |
|---|---|---|
| 6342S_N_465 | ATGATGGAGAATCAGGAGAACAACATGAACAACAGAAAGAACTACAGAGA | SEQ ID NO: |
| _K | GGCCGTGAACACCATGGGCAAGAAGGAATTCACCCTGATCAAGATGCAGG | 19222 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAAGGAGAT | |
| | CAGCGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_N_465 | ATGATGGAGAATCAGGAGAACAACATGAACAACAGAAAGAACTACAGAGA | SEQ ID NO: |
| _Q | GGCCGTGAACACCATGGGCAAGAAGGAATTCACCCTGATCAAGATGCAGG | 19223 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCCAGGAGAT | |
| | CAGCGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_N_465 | ATGATGGAGAATCAGGAGAACAACATGAACAACAGAAAGAACTACAGAGA | SEQ ID NO: |
| _R | GGCCGTGAACACCATGGGCAAGAAGGAATTCACCCTGATCAAGATGCAGG | 19224 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCCGGGAGAT | |
| | CAGCGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_E_466 | ATGATGGAGAATCAGGAGAACAACATGAACAACAGAAAGAACTACAGAGA | SEQ ID NO: |
| _K | GGCCGTGAACACCATGGGCAAGAAGGAATTCACCCTGATCAAGATGCAGG | 19225 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACAAGAT | |
| | CAGCGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_E_466 | ATGATGGAGAATCAGGAGAACAACATGAACAACAGAAAGAACTACAGAGA | SEQ ID NO: |
| _N | GGCCGTGAACACCATGGGCAAGAAGGAATTCACCCTGATCAAGATGCAGG | 19226 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACAACAT | |
| | CAGCGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_E_466 | ATGATGGAGAATCAGGAGAACAACATGAACAACAGAAAGAACTACAGAGA | SEQ ID NO: |
| _O | GGCCGTGAACACCATGGGCAAGAAGGAATTCACCCTGATCAAGATGCAGG | 19227 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACCAGAT | |
| | CAGCGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_E_466 | ATGATGGAGAATCAGGAGAACAACATGAACAACAGAAAGAACTACAGAGA | SEQ ID NO: |
| _R | GGCCGTGAACACCATGGGCAAGAAGGAATTCACCCTGATCAAGATGCAGG | 19228 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACCGGAT | |
| | CAGCGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_S_468 | ATGATGGAGAATCAGGAGAACAACATGAACAACAGAAAGAACTACAGAGA | SEQ ID NO: |
| _K | GGCCGTGAACACCATGGGCAAGAAGGAATTCACCCTGATCAAGATGCAGG | 19229 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAAGGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_S_468 | ATGATGGAGAATCAGGAGAACAACATGAACAACAGAAAGAACTACAGAGA | SEQ ID NO: |
| _N | GGCCGTGAACACCATGGGCAAGAAGGAATTCACCCTGATCAAGATGCAGG | 19230 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAACGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_S_468 | ATGATGGAGAATCAGGAGAACAACATGAACAACAGAAAGAACTACAGAGA | SEQ ID NO: |
| _Q | GGCCGTGAACACCATGGGCAAGAAGGAATTCACCCTGATCAAGATGCAGG | 19231 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CCAGGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_S_468 | ATGATGGAGAATCAGGAGAACAACATGAACAACAGAAAGAACTACAGAGA | SEQ ID NO: |
| _R | GGCCGTGAACACCATGGGCAAGAAGGAATTCACCCTGATCAAGATGCAGG | 19232 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CCGGGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_M_470 | ATGATGGAGAATCAGGAGAACAACATGAACAACAGAAAGAACTACAGAGA | SEQ ID NO: |
| _K | GGCCGTGAACACCATGGGCAAGAAGGAATTCACCCTGATCAAGATGCAGG | 19233 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCAAGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_M_470 | ATGATGGAGAATCAGGAGAACAACATGAACAACAGAAAGAACTACAGAGA | SEQ ID NO: |
| _N | GGCCGTGAACACCATGGGCAAGAAGGAATTCACCCTGATCAAGATGCAGG | 19234 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCAACACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_M_470 | ATGATGGAGAATCAGGAGAACAACATGAACAACAGAAAGAACTACAGAGA | SEQ ID NO: |
| _Q | GGCCGTGAACACCATGGGCAAGAAGGAATTCACCCTGATCAAGATGCAGG | 19235 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCCAGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_M_470 | ATGATGGAGAATCAGGAGAACAACATGAACAACAGAAAGAACTACAGAGA | SEQ ID NO: |
| _R | GGCCGTGAACACCATGGGCAAGAAGGAATTCACCCTGATCAAGATGCAGG | 19236 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCCGGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_S_472_ | ATGATGGAGAATCAGGAGAACAACATGAACAACAGAAAGAACTACAGAGA | SEQ ID NO: |
| G | GGCCGTGAACACCATGGGCAAGAAGGAATTCACCCTGATCAAGATGCAGG | 19237 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCGGCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_S_472_P | ATGATGGAGAATCAGGAGAACAACATGAACAACAGAAAGAACTACAGAGA | SEQ ID NO: |
| | GGCCGTGAACACCATGGGCAAGAAGGAATTCACCCTGATCAAGATGCAGG | 19238 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCCCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_W_473 | ATGATGGAGAATCAGGAGAACAACATGAACAACAGAAAGAACTACAGAGA | SEQ ID NO: |
| _F | GGCCGTGAACACCATGGGCAAGAAGGAATTCACCCTGATCAAGATGCAGG | 19239 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTTCGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_W_473 | ATGATGGAGAATCAGGAGAACAACATGAACAACAGAAAGAACTACAGAGA | SEQ ID NO: |
| _K | GGCCGTGAACACCATGGGCAAGAAGGAATTCACCCTGATCAAGATGCAGG | 19240 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCAAGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_W_473_ | ATGATGGAGAATCAGGAGAACAACATGAACAACAGAAAGAACTACAGAGA | SEQ ID NO: |
| R | GGCCGTGAACACCATGGGCAAGAAGGAATTCACCCTGATCAAGATGCAGG | 19241 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCCGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_W_473_ | ATGATGGAGAATCAGGAGAACAACATGAACAACAGAAAGAACTACAGAGA | SEQ ID NO: |
| Y | GGCCGTGAACACCATGGGCAAGAAGGAATTCACCCTGATCAAGATGCAGG | 19242 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTACGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_K_ | ATGATGGAGAATCAGGAGAACAACATGAACAACAGAAAGAACTACAGAGA | SEQ ID NO: |
| 475_N | GGCCGTGAACACCATGGGCAAGAAGGAATTCACCCTGATCAAGATGCAGG | 19243 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAACTCCGTCAACGAGGAGAGATACAAGG | |
| | ACATCATCAACGATGCCATGAACCTGATCAACAACccaccaaagaagaaa | |
| | agaaaggtctga | |
| 6342S_K_ | ATGATGGAGAATCAGGAGAACAACATGAACAACAGAAAGAACTACAGAGA | SEQ ID NO: |
| 475_Q | GGCCGTGAACACCATGGGCAAGAAGGAATTCACCCTGATCAAGATGCAGG | 19244 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGCAGTCCGTCAACGAGGAGAGATACAAGG | |
| | ACATCATCAACGATGCCATGAACCTGATCAACAACccaccaaagaagaaa | |
| | agaaaggtctga | |
| 6342S_K_475_ | ATGATGGAGAATCAGGAGAACAACATGAACAACAGAAAGAACTACAGAGA | SEQ ID NO: |
| R | GGCCGTGAACACCATGGGCAAGAAGGAATTCACCCTGATCAAGATGCAGG | 19245 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGCGGTCCGTCAACGAGGAGAGATACAAGG | |
| | ACATCATCAACGATGCCATGAACCTGATCAACAACccaccaaagaagaaa | |
| | agaaaggtctga | |
| 6342S_S_476_ | ATGATGGAGAATCAGGAGAACAACATGAACAACAGAAAGAACTACAGAGA | SEQ ID NO: |
| K | GGCCGTGAACACCATGGGCAAGAAGGAATTCACCCTGATCAAGATGCAGG | 19246 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGAAGGTCAACGAGGAGAGATACAAGG | |
| | ACATCATCAACGATGCCATGAACCTGATCAACAACccaccaaagaagaaa | |
| | agaaaggtctga | |
| 6342S_S_476_ | ATGATGGAGAATCAGGAGAACAACATGAACAACAGAAAGAACTACAGAGA | SEQ ID NO: |
| N | GGCCGTGAACACCATGGGCAAGAAGGAATTCACCCTGATCAAGATGCAGG | 19247 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGAACGTCAACGAGGAGAGATACAAGG | |
| | ACATCATCAACGATGCCATGAACCTGATCAACAACccaccaaagaagaaa | |
| | agaaaggtctga | |
| 6342S_S_476_ | ATGATGGAGAATCAGGAGAACAACATGAACAACAGAAAGAACTACAGAGA | SEQ ID NO: |
| Q | GGCCGTGAACACCATGGGCAAGAAGGAATTCACCCTGATCAAGATGCAGG | 19248 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGCAGGTCAACGAGGAGAGATACAAGG | |
| | ACATCATCAACGATGCCATGAACCTGATCAACAACccaccaaagaagaaa | |
| | agaaaggtctga | |
| 6342S S_476_ | ATGATGGAGAATCAGGAGAACAACATGAACAACAGAAAGAACTACAGAGA | SEQ ID NO: |
| R | GGCCGTGAACACCATGGGCAAGAAGGAATTCACCCTGATCAAGATGCAGG | 19249 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGCGGGTCAACGAGGAGAGATACAAGG | |
| | ACATCATCAACGATGCCATGAACCTGATCAACAACccaccaaagaagaaa | |
| | agaaaggtctga | |
| 6342S_V_477_ | ATGATGGAGAATCAGGAGAACAACATGAACAACAGAAAGAACTACAGAGA | SEQ ID NO: |
| F | GGCCGTGAACACCATGGGCAAGAAGGAATTCACCCTGATCAAGATGCAGG | 19250 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGTCCTTCAACGAGGAGAGATACAAGG | |
| | ACATCATCAACGATGCCATGAACCTGATCAACAACccaccaaagaagaaa | |
| | agaaaggtctga | |
| 6342S_V_477_I | ATGATGGAGAATCAGGAGAACAACATGAACAACAGAAAGAACTACAGAGA | SEQ ID NO: |
| | GGCCGTGAACACCATGGGCAAGAAGGAATTCACCCTGATCAAGATGCAGG | 19251 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGTCCATCAACGAGGAGAGATACAAGG | |
| | ACATCATCAACGATGCCATGAACCTGATCAACAACccaccaaagaagaaa | |
| | agaaaggtctga | |
| 6342S_V_477_ | ATGATGGAGAATCAGGAGAACAACATGAACAACAGAAAGAACTACAGAGA | SEQ ID NO: |
| L | GGCCGTGAACACCATGGGCAAGAAGGAATTCACCCTGATCAAGATGCAGG | 19252 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGTCCCTGAACGAGGAGAGATACAAGG | |
| | ACATCATCAACGATGCCATGAACCTGATCAACAACccaccaaagaagaaa | |
| | agaaaggtctga | |
| 6342S_V_477_ | ATGATGGAGAATCAGGAGAACAACATGAACAACAGAAAGAACTACAGAGA | SEQ ID NO: |
| W | GGCCGTGAACACCATGGGCAAGAAGGAATTCACCCTGATCAAGATGCAGG | 19253 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGTCCTGGAACGAGGAGAGATACAAGG | |
| | ACATCATCAACGATGCCATGAACCTGATCAACAACccaccaaagaagaaa | |
| | agaaaggtctga | |
| 6342S_V_265_ | ATGATGGAGAATCAGGAGAACAACATGAACAACAGAAAGAACTACAGAGA | SEQ ID NO: |
| P | GGCCGTGAACACCATGGGCAAGAAGGAATTCACCCTGATCAAGATGCAGG | 19254 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGCCTCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAC | |
| | AAGGTGAAGAACAGCGGCGAGATCCTGAGCAACAAGGTGATCAACGAGAT | |
| | CAGCGGCATGACCTCCTGGGTGAAGTCCGTCAACGAGGAGAGATACAAGG | |
| | ACATCATCAACGATGCCATGAACCTGATCAACAACccaccaaagaagaaa | |
| | agaaaggtctga | |
| 6342S_M470K | ATGATGGAGAATCAGGAGAACAACATGAACAACAGAAAGAACTACAGAGA | SEQ ID NO: |
| _V477W | GGCCGTGAACACCATGGGCAAGAAGGAATTCACCCTGATCAAGATGCAGG | 19255 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGGTGCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAT | |
| | AAGGTTAAGAATAGTGGAGAAATTCTAAGTAATAAAGTCATAAACGAGAT | |
| | CAGCGGCAAGACCTCCTGGGTGAAGTCCTTCAACGAGGAGAGATACAAGG | |
| | ACATCATCAACGATGCCATGAACCTGATCAACAACCCACCAAAGAAGAAA | |
| | AGAAAGGTCTGA | |
| 6342S_M470K_ | ATGATGGAGAATCAGGAGAACAACATGAACAACAGAAAGAACTACAGAGA | SEQ ID NO: |
| V477W_V265P | GGCCGTGAACACCATGGGCAAGAAGGAATTCACCCTGATCAAGATGCAGG | 19256 |
| | AGTACGGCTTCTGGCCCAAGAACCTGCCTACTCCCTACGAGAGGCAGGAA | |
| | AGCGAGACCGAGGAGCAGTACAAGGAGAGAAAGTGCCTGCTCGAGAAGTA | |
| | CGAGAAGGTGATCGACGAGATCAGCAAGCTGTACGAAGAGAAGGACAAGA | |
| | TCAACCTGAAGCTGAGGGAGCTCCAGAAGAAGTACGACGAGACCTGGGAC | |
| | TATGAGCGCATCCGGCTGGATGTGAGCAAGACCATCATGCAGGAGAGCAT | |
| | CGAGAGGAGAGCTGAAAGAAAGCGCCAGCGGGAGCTGGAGAAGGAGCAGC | |
| | GCTCCGAGGAGTGGAAGAAGGAGAAGGAGAACAAGATCGTCTTCATCGGC | |
| | AAGGGGTACAGCAGCCTGCTGTACGACAAGGAGACCGACGAGAACAAGCT | |
| | GCTGCTGCAGGAGCTCCCTATCATCAAGGATGATAAGGAGCTGGCCAACT | |
| | TCCTGGGCATCGAGTACAAGAAGCTGAGATTCCTGGTGTACCACCGGGAC | |
| | GTGATCTCCGTGGACAACTACCACAGATACACCATCCCCAAGAAGAAGGG | |
| | CGGCGTGAGAAACATCGCCGCTCCCAAGAGCATCCTGAAGAACTCCCAGA | |
| | GGATTATCCTCGAGGAGATTCTGAGCAAGATCCCCACCTCCAACTACAGC | |
| | CACGGCTTCCTGAAGGGGAAGTCCGTGGTGAGCGCCGCCAAGCCCCACGT | |
| | GAAGAAACCCGACCTCCTGATCAACATCGATCTGGAGGACTTCTTCCCCA | |
| | CCATCACCTTCGAGAGGGTGAGAGGCATGTTCAAGAGCTTCGGCTACAGC | |
| | GGATACGTGGCCAGCATGCTGGCCATGATCTGCACCTACTGCGAGAGGAT | |
| | GAAGGTGGAGGTGAGGGGCGAGGAGAAGTACGTCAAGATCAGCGACAGAA | |
| | TTCTGCCCCAGGGCAGCCCTGCCAGCCCCATGATCACCAACATCATCTGC | |
| | GTGAAGCTGGACAAGAGACTGAACGGCCTGAGCACCAAGTATGACTTCAT | |
| | TTATACTCGGTACGCTGACGACATGTCGTTCAGCTTCACCGGCGACATCA | |
| | ACGAACTGAGCGTCGGCAGCTTCATGGGCCTGGTGAGCAAGATCGTGAAG | |
| | GAAGAGGGCTTCAACATCAACAAGGACAAGACCAAGTTCCTGAGAAAGAA | |
| | CAACAGACAGTGCATCACCGGCATCGTGATCAACAACGAGGAGATCGGCG | |
| | TGCCCAAGAAGTGGATCAAGATCCTGAGAGCCGCCATCTACAACGCCAAT | |
| | AAGGTTAAGAATAGTGGAGAAATTCTAAGTAATAAAGTCATAAACGAGAT | |
| | CAGCGGCAAGACCTCCTGGGTGAAGTCCTTCAACGAGGAGAGATACAAGG | |
| | ACATCATCAACGATGCCATGAACCTGATCAACAACCCACCAAAGAAGAAA | |
| | AGAAAGGTCTGA | |
| 6342S_ | ATGGAGAGGAGAGCTGAAAGAAAGCGCCAGCGGGAGCTGGAGAAGGAGCA | SEQ ID NO: |
| ntermtrunc_ | GCGCTCCGAGGAGTGGAAGAAGGAGAAGGAGAACAAGATCGTCTTCATCG | 19257 |
| whelix | GCAAGGGGTACAGCAGCCTGCTGTACGACAAGGAGACCGACGAGAACAAG | |
| | CTGCTGCTGCAGGAGCTCCCTATCATCAAGGATGATAAGGAGCTGGCCAA | |
| | CTTCCTGGGCATCGAGTACAAGAAACTGAGATTCCTGGTGTACCACCGGG | |
| | ACGTGATCTCCGTGGACAACTACCACAGATACACCATCCCCAAGAAGAAG | |
| | GGCGGCGTGAGAAACATCGCCGCTCCCAAGAGCATCCTGAAGAACTCCCA | |
| | GAGGATTATCCTCGAGGAGATTCTGAGCAAGATCCCCACCTCCAACTACA | |
| | GCCACGGCTTCCTGAAGGGGAAGTCCGTGGTGAGCGCCGCCAAGGTGCAC | |
| | GTGAAGAAACCCGACCTCCTGATCAACATCGATCTGGAGGACTTCTTCCC | |
| | CACCATCACCTTCGAGAGGGTGAGAGGCATGTTCAAGAGCTTCGGCTACA | |
| | GCGGATACGTGGCCAGCATGCTGGCCATGATCTGCACCTACTGCGAGAGG | |
| | ATGAAGGTGGAGGTGAGGGGCGAGGAGAAGTACGTCAAGATCAGCGACAG | |
| | AATTCTGCCCCAGGGCAGCCCTGCCAGCCCCATGATCACCAACATCATCT | |
| | GCGTGAAGCTGGACAAGAGACTGAACGGCCTGAGCACCAAGTATGACTTC | |
| | ATTTATACTCGGTACGCTGACGACATGTCGTTCAGCTTCACCGGCGACAT | |
| | CAACGAACTGAGCGTCGGCAGCTTCATGGGCCTGGTGAGCAAGATCGTGA | |
| | AGGAAGAGGGCTTCAACATCAACAAGGACAAGACCAAGTTCCTGAGAAAG | |
| | AACAACAGACAGTGCATCACCGGCATCGTGATCAACAACGAGGAGATCGG | |
| | CGTGCCCAAGAAGTGGATCAAGATCCTGAGAGCCGCCATCTACAACGCCA | |
| | ACAAGGTGAAGAACAGCGGCGAGATCCTGAGCAATAAGGTGATCAACGAG | |
| | ATCAGCGGCATGACCTCCTGGGTGAAGTCCGTCAACGAAGAGAGATACAA | |
| | GGACaTTATCAACGATGCCATGAACCTGATCAACAACccaccaaagaaga | |
| | aaCGGaaggtctga | |
| Sso7d-PASTE | ATGACCGTCAAGTTCAAGTACAAGGGTGAGGAACTTGAAGTTGATATTAG | SEQ ID NO: |
| (v2)_WT 6342S | CAAAATCAAGAAGGTTTGGCGCGTTGGTAAAATGATATCTTTTACTTATG | 19258 |
| | ACGACAACGGCAAGACAGGTAGAGGGGCAGTGTCTGAGAAAGACGCCCCC | |
| | AAGGAGCTGTTGCAAATGTTGGAAAAGTCTGGGAAAAAGTCTGGCGGCTC | |
| | AAAAAGAACCGCCGACGGCAGCGAATTCGAGCCCAAGAAGAAGAGGAAAG | |
| | TCGGGACAGGTGGCGGTGGTGTCGAGAATCAGGAGAACAACATGAACAAC | |
| | AGAAAGAACTACAGAGAGGCCGTGAACACCATGGGCAAGAAGGAATTCAC | |
| | CCTGATCAAGATGCAGGAGTACGGCTTCTGGCCCAAGAACCTGCCTACTC | |
| | CCTACGAGAGGCAGGAAAGCGAGACCGAGGAGCAGTACAAGGAGAGAAAG | |
| | TGCCTGCTCGAGAAGTACGAGAAGGTGATCGACGAGATCAGCAAGCTGTA | |
| | CGAAGAGAAGGACAAGATCAACCTGAAGCTGAGGGAGCTCCAGAAGAAGT | |
| | ACGACGAGACCTGGGACTATGAGCGCATCCGGCTGGATGTGAGCAAGACC | |
| | ATCATGCAGGAGAGCATCGAGAGGAGAGCTGAAAGAAAGCGCCAGCGGGA | |
| | GCTGGAGAAGGAGCAGCGCTCCGAGGAGTGGAAGAAGGAGAAGGAGAACA | |
| | AGATCGTCTTCATCGGCAAGGGGTACAGCAGCCTGCTGTACGACAAGGAG | |
| | ACCGACGAGAACAAGCTGCTGCTGCAGGAGCTCCCTATCATCAAGGATGA | |
| | TAAGGAGCTGGCCAACTTCCTGGGCATCGAGTACAAGAAGCTGAGATTCC | |
| | TGGTGTACCACCGGGACGTGATCTCCGTGGACAACTACCACAGATACACC | |
| | ATCCCCAAGAAGAAGGGCGGCGTGAGAAACATCGCCGCTCCCAAGAGCAT | |
| | CCTGAAGAACTCCCAGAGGATTATCCTCGAGGAGATTCTGAGCAAGATCC | |
| | CCACCTCCAACTACAGCCACGGCTTCCTGAAGGGGAAGTCCGTGGTGAGC | |
| | GCCGCCAAGGTGCACGTGAAGAAACCCGACCTCCTGATCAACATCGATCT | |
| | GGAGGACTTCTTCCCCACCATCACCTTCGAGAGGGTGAGAGGCATGTTCA | |
| | AGAGCTTCGGCTACAGCGGATACGTGGCCAGCATGCTGGCCATGATCTGC | |
| | ACCTACTGCGAGAGGATGAAGGTGGAGGTGAGGGGCGAGGAGAAGTACGT | |
| | CAAGATCAGCGACAGAATTCTGCCCCAGGGCAGCCCTGCCAGCCCCATGA | |
| | TCACCAACATCATCTGCGTGAAGCTGGACAAGAGACTGAACGGCCTGAGC | |
| | ACCAAGTATGACTTCATTTATACTCGGTACGCTGACGACATGTCGTTCAG | |
| | CTTCACCGGCGACATCAACGAACTGAGCGTCGGCAGCTTCATGGGCCTGG | |
| | TGAGCAAGATCGTGAAGGAAGAGGGCTTCAACATCAACAAGGACAAGACC | |
| | AAGTTCCTGAGAAAGAACAACAGACAGTGCATCACCGGCATCGTGATCAA | |
| | CAACGAGGAGATCGGCGTGCCCAAGAAGTGGATCAAGATCCTGAGAGCCG | |
| | CCATCTACAACGCCAACAAGGTGAAGAACAGCGGCGAGATCCTGAGCAAC | |
| | AAGGTGATCAACGAGATCAGCGGCATGACCTCCTGGGTGAAGTCCGTCAA | |
| | CGAGGAGAGATACAAGGACATCATCAACGATGCCATGAACCTGATCAACA | |
| | AC | |
| Sso7d- | ATGACAGTGAAGTTCAAGTACAAGGGCGAGGAAAAGGAAGTCGACATCAG | SEQ ID NO: |
| ncbiprotein | CAAGATCAAGAAAGTGTGGCGGGTGGGCAAGATGATCAGCTTCACCTACG | 19259 |
| (v1)_WT 6342S | ACGAGGGCGGAGGAAAGACCGGCAGAGGCGCCGTGTCTGAGAAAGATGCC | |
| | CCTAAGGAGCTGCTGCAGATGCTGGAAAAACAGAAGAAGCCCAAGAAGAA | |
| | GAGGAAAGTCGGGACAGGTGGCGGTGGTGTCGAGAATCAGGAGAACAACA | |
| | TGAACAACAGAAAGAACTACAGAGAGGCCGTGAACACCATGGGCAAGAAG | |
| | GAATTCACCCTGATCAAGATGCAGGAGTACGGCTTCTGGCCCAAGAACCT | |
| | GCCTACTCCCTACGAGAGGCAGGAAAGCGAGACCGAGGAGCAGTACAAGG | |
| | AGAGAAAGTGCCTGCTCGAGAAGTACGAGAAGGTGATCGACGAGATCAGC | |
| | AAGCTGTACGAAGAGAAGGACAAGATCAACCTGAAGCTGAGGGAGCTCCA | |
| | GAAGAAGTACGACGAGACCTGGGACTATGAGCGCATCCGGCTGGATGTGA | |
| | GCAAGACCATCATGCAGGAGAGCATCGAGAGGAGAGCTGAAAGAAAGCGC | |
| | CAGCGGGAGCTGGAGAAGGAGCAGCGCTCCGAGGAGTGGAAGAAGGAGAA | |
| | GGAGAACAAGATCGTCTTCATCGGCAAGGGGTACAGCAGCCTGCTGTACG | |
| | ACAAGGAGACCGACGAGAACAAGCTGCTGCTGCAGGAGCTCCCTATCATC | |
| | AAGGATGATAAGGAGCTGGCCAACTTCCTGGGCATCGAGTACAAGAAGCT | |
| | GAGATTCCTGGTGTACCACCGGGACGTGATCTCCGTGGACAACTACCACA | |
| | GATACACCATCCCCAAGAAGAAGGGCGGCGTGAGAAACATCGCCGCTCCC | |
| | AAGAGCATCCTGAAGAACTCCCAGAGGATTATCCTCGAGGAGATTCTGAG | |
| | CAAGATCCCCACCTCCAACTACAGCCACGGCTTCCTGAAGGGGAAGTCCG | |
| | TGGTGAGCGCCGCCAAGGTGCACGTGAAGAAACCCGACCTCCTGATCAAC | |
| | ATCGATCTGGAGGACTTCTTCCCCACCATCACCTTCGAGAGGGTGAGAGG | |
| | CATGTTCAAGAGCTTCGGCTACAGCGGATACGTGGCCAGCATGCTGGCCA | |
| | TGATCTGCACCTACTGCGAGAGGATGAAGGTGGAGGTGAGGGGCGAGGAG | |
| | AAGTACGTCAAGATCAGCGACAGAATTCTGCCCCAGGGCAGCCCTGCCAG | |
| | CCCCATGATCACCAACATCATCTGCGTGAAGCTGGACAAGAGACTGAACG | |
| | GCCTGAGCACCAAGTATGACTTCATTTATACTCGGTACGCTGACGACATG | |
| | TCGTTCAGCTTCACCGGCGACATCAACGAACTGAGCGTCGGCAGCTTCAT | |
| | GGGCCTGGTGAGCAAGATCGTGAAGGAAGAGGGCTTCAACATCAACAAGG | |
| | ACAAGACCAAGTTCCTGAGAAAGAACAACAGACAGTGCATCACCGGCATC | |
| | GTGATCAACAACGAGGAGATCGGCGTGCCCAAGAAGTGGATCAAGATCCT | |
| | GAGAGCCGCCATCTACAACGCCAACAAGGTGAAGAACAGCGGCGAGATCC | |
| | TGAGCAACAAGGTGATCAACGAGATCAGCGGCATGACCTCCTGGGTGAAG | |
| | TCCGTCAACGAGGAGAGATACAAGGACATCATCAACGATGCCATGAACCT | |
| | GATCAACAAC | |
| Sac7d- | ATGGTGAAGGTGAAATTCAAGTACAAGGGCGAGGAAAAAGAGGTGGATAC | SEQ ID NO: |
| ncbiprotein | AAGCAAGATCAAGAAAGTGTGGAGAGTGGGAAAGATGGTCAGCTTCACCT | 19260 |
| (v2)_WT 6342S | ACGACGACAACGGCAAGACCGGCAGAGGCGCCGTGTCTGAGAAGGACGCC | |
| | CCTAAGGAACTGCTGGATATGCTGGCTAGAGCCGAGCGGGAAAAGAAGCC | |
| | CAAGAAGAAGAGGAAAGTCGGGACAGGTGGCGGTGGTGTCGAGAATCAGG | |
| | AGAACAACATGAACAACAGAAAGAACTACAGAGAGGCCGTGAACACCATG | |
| | GGCAAGAAGGAATTCACCCTGATCAAGATGCAGGAGTACGGCTTCTGGCC | |
| | CAAGAACCTGCCTACTCCCTACGAGAGGCAGGAAAGCGAGACCGAGGAGC | |
| | AGTACAAGGAGAGAAAGTGCCTGCTCGAGAAGTACGAGAAGGTGATCGAC | |
| | GAGATCAGCAAGCTGTACGAAGAGAAGGACAAGATCAACCTGAAGCTGAG | |
| | GGAGCTCCAGAAGAAGTACGACGAGACCTGGGACTATGAGCGCATCCGGC | |
| | TGGATGTGAGCAAGACCATCATGCAGGAGAGCATCGAGAGGAGAGCTGAA | |
| | AGAAAGCGCCAGCGGGAGCTGGAGAAGGAGCAGCGCTCCGAGGAGTGGAA | |
| | GAAGGAGAAGGAGAACAAGATCGTCTTCATCGGCAAGGGGTACAGCAGCC | |
| | TGCTGTACGACAAGGAGACCGACGAGAACAAGCTGCTGCTGCAGGAGCTC | |
| | CCTATCATCAAGGATGATAAGGAGCTGGCCAACTTCCTGGGCATCGAGTA | |
| | CAAGAAGCTGAGATTCCTGGTGTACCACCGGGACGTGATCTCCGTGGACA | |
| | ACTACCACAGATACACCATCCCCAAGAAGAAGGGCGGCGTGAGAAACATC | |
| | GCCGCTCCCAAGAGCATCCTGAAGAACTCCCAGAGGATTATCCTCGAGGA | |
| | GATTCTGAGCAAGATCCCCACCTCCAACTACAGCCACGGCTTCCTGAAGG | |
| | GGAAGTCCGTGGTGAGCGCCGCCAAGGTGCACGTGAAGAAACCCGACCTC | |
| | CTGATCAACATCGATCTGGAGGACTTCTTCCCCACCATCACCTTCGAGAG | |
| | GGTGAGAGGCATGTTCAAGAGCTTCGGCTACAGCGGATACGTGGCCAGCA | |
| | TGCTGGCCATGATCTGCACCTACTGCGAGAGGATGAAGGTGGAGGTGAGG | |
| | GGCGAGGAGAAGTACGTCAAGATCAGCGACAGAATTCTGCCCCAGGGCAG | |
| | CCCTGCCAGCCCCATGATCACCAACATCATCTGCGTGAAGCTGGACAAGA | |
| | GACTGAACGGCCTGAGCACCAAGTATGACTTCATTTATACTCGGTACGCT | |
| | GACGACATGTCGTTCAGCTTCACCGGCGACATCAACGAACTGAGCGTCGG | |
| | CAGCTTCATGGGCCTGGTGAGCAAGATCGTGAAGGAAGAGGGCTTCAACA | |
| | TCAACAAGGACAAGACCAAGTTCCTGAGAAAGAACAACAGACAGTGCATC | |
| | ACCGGCATCGTGATCAACAACGAGGAGATCGGCGTGCCCAAGAAGTGGAT | |
| | CAAGATCCTGAGAGCCGCCATCTACAACGCCAACAAGGTGAAGAACAGCG | |
| | GCGAGATCCTGAGCAACAAGGTGATCAACGAGATCAGCGGCATGACCTCC | |
| | TGGGTGAAGTCCGTCAACGAGGAGAGATACAAGGACATCATCAACGATGC | |
| | CATGAACCTGATCAACAAC | |
| Sac7d-ncbigene | ATGGTCAAGGTGAAGTTCAAGTACAAGGGCGAGGAAAAAGAGGTGGATAC | SEQ ID NO: |
| (v1)_WT 6342S | AAGCAAGATCAAGAAAGTGTGGAGAGTGGGAAAAATGGTGAGCTTCACCT | 19261 |
| | ACGACGACAACGGCAAGACCGGCAGAGGCGCCGTGTCTGAGAAGGACGCC | |
| | CCTAAGGAACTGCTGGATATGCTGGCTAGAGCCGAGCGGGAAAAGAAGCC | |
| | CAAGAAGAAGAGGAAAGTCGGGACAGGTGGCGGTGGTGTCGAGAATCAGG | |
| | AGAACAACATGAACAACAGAAAGAACTACAGAGAGGCCGTGAACACCATG | |
| | GGCAAGAAGGAATTCACCCTGATCAAGATGCAGGAGTACGGCTTCTGGCC | |
| | CAAGAACCTGCCTACTCCCTACGAGAGGCAGGAAAGCGAGACCGAGGAGC | |
| | AGTACAAGGAGAGAAAGTGCCTGCTCGAGAAGTACGAGAAGGTGATCGAC | |
| | GAGATCAGCAAGCTGTACGAAGAGAAGGACAAGATCAACCTGAAGCTGAG | |
| | GGAGCTCCAGAAGAAGTACGACGAGACCTGGGACTATGAGCGCATCCGGC | |
| | TGGATGTGAGCAAGACCATCATGCAGGAGAGCATCGAGAGGAGAGCTGAA | |
| | AGAAAGCGCCAGCGGGAGCTGGAGAAGGAGCAGCGCTCCGAGGAGTGGAA | |
| | GAAGGAGAAGGAGAACAAGATCGTCTTCATCGGCAAGGGGTACAGCAGCC | |
| | TGCTGTACGACAAGGAGACCGACGAGAACAAGCTGCTGCTGCAGGAGCTC | |
| | CCTATCATCAAGGATGATAAGGAGCTGGCCAACTTCCTGGGCATCGAGTA | |
| | CAAGAAGCTGAGATTCCTGGTGTACCACCGGGACGTGATCTCCGTGGACA | |
| | ACTACCACAGATACACCATCCCCAAGAAGAAGGGCGGCGTGAGAAACATC | |
| | GCCGCTCCCAAGAGCATCCTGAAGAACTCCCAGAGGATTATCCTCGAGGA | |
| | GATTCTGAGCAAGATCCCCACCTCCAACTACAGCCACGGCTTCCTGAAGG | |
| | GGAAGTCCGTGGTGAGCGCCGCCAAGGTGCACGTGAAGAAACCCGACCTC | |
| | CTGATCAACATCGATCTGGAGGACTTCTTCCCCACCATCACCTTCGAGAG | |
| | GGTGAGAGGCATGTTCAAGAGCTTCGGCTACAGCGGATACGTGGCCAGCA | |
| | TGCTGGCCATGATCTGCACCTACTGCGAGAGGATGAAGGTGGAGGTGAGG | |
| | GGCGAGGAGAAGTACGTCAAGATCAGCGACAGAATTCTGCCCCAGGGCAG | |
| | CCCTGCCAGCCCCATGATCACCAACATCATCTGCGTGAAGCTGGACAAGA | |
| | GACTGAACGGCCTGAGCACCAAGTATGACTTCATTTATACTCGGTACGCT | |
| | GACGACATGTCGTTCAGCTTCACCGGCGACATCAACGAACTGAGCGTCGG | |
| | CAGCTTCATGGGCCTGGTGAGCAAGATCGTGAAGGAAGAGGGCTTCAACA | |
| | TCAACAAGGACAAGACCAAGTTCCTGAGAAAGAACAACAGACAGTGCATC | |
| | ACCGGCATCGTGATCAACAACGAGGAGATCGGCGTGCCCAAGAAGTGGAT | |
| | CAAGATCCTGAGAGCCGCCATCTACAACGCCAACAAGGTGAAGAACAGCG | |
| | GCGAGATCCTGAGCAACAAGGTGATCAACGAGATCAGCGGCATGACCTCC | |
| | TGGGTGAAGTCCGTCAACGAGGAGAGATACAAGGACATCATCAACGATGC | |
| | CATGAACCTGATCAACAAC | |

### Plasmid Sequences

| Name | Desc | Sequence | SEQ ID NO: |
|---|---|---|---|
| Eco1 RT | Coding sequence only. | AUGAAAUCUGCAGAGUAUCUGAAUACGUUCCGCCUU | SEQ ID NO: |
| | RNA was produced from a | AGGAAUUUGGGCCUCCCCGUGAUGAACAAUCUCCAC | 19262 |
| | CRO and proprietary 5′ cap, | GAUAUGAGCAAGGCGACUCGAAUAUCCGUGGAAACG | |
| | 5′ UTR, 3′ UTR and | CUGAGACUGCUCAUCUAUACAGCAGACUUUCGGUAC | |
| | encoded poly A of around | AGGAUCUACACGGUCGAAAAGAAGGGGCCUGAGAAA | |
| | 120 bases were added. Full | CGCAUGCGAACAAUUUAUCAACCUAGCCGAGAGCUC | |
| | substitution with 1N- | AAGGCGUUGCAGGGCUGGGUUCUUCGAAACAUCCUU | |
| | methyl-pseudouridine | GACAAACUCUCAUCAUCACCCUUUAGUAUUGGGUUU | |
| | | GAAAAGCACCAAAGCAUCCUUAACAACGCGACGCCA | |
| | | CACAUAGGUGCCAAUUUCAUAUUGAACAUCGACUUG | |
| | | GAGGAUUUUUUUCCGAGCCUCACAGCCAAUAAAGUG | |
| | | UUCGGUGUUUUUCACAGUCUUGGGUACAAUCGCCUU | |
| | | AUUAGUUCCGUUCUUACCAAGAUUUGUUGUUACAAG | |
| | | AAUCUCUUGCCCCAGGGAGCACCCAGCAGUCCGAAA | |
| | | UUGGCGAAUUUGAUUUGUUCCAAGCUCGAUUAUCGA | |
| | | AUACAAGGGUACGCGGGCAGCCGGGGACUCAUCUAU | |
| | | ACCCGCUACGCAGACGAUCUUACGCUGUCUGCCCAA | |
| | | UCAAUGAAGAAGGUCGUAAAGGCGCGGGAUUUCUUG | |
| | | UUUUCUAUCAUCCCGUCCGAGGGCUUGGUAAUUAAU | |
| | | UCCAAAAAGACUUGUAUCUCAGGACCACGAUCUCAG | |
| | | CGAAAAGUGACAGGACUCGUCAUUUCUCAAGAAAAA | |
| | | GUCGGUAUAGGGAGAGAGAAGUAUAAGGAAAUCCGC | |
| | | GCGAAGAUCCACCACAUAUUCUGUGGCAAGAGCAGC | |
| | | GAGAUAGAACACGUCCGAGGCUGGUUGUCCUUCAUA | |
| | | CUGAGCGUGGACUCAAAAAGCCACCGCCGGUUGAUC | |
| | | ACCUAUAUUUCAAAACUGGAAAAGAAAUAUGGAAAG | |
| | | AACCCACUCAACAAAGCUAAAACACCACCAAAGAAG | |
| | | AAAAGAAAGGUCUGA | |
| Eco1 ncRNA- | ncRNA-sgRNA fusion (6 | GAAAUGAUAAGAUUCCGUAUGCGCACCCUUAGCGAG | SEQ ID NO: |
| sgRNA | bp substition in EMX1 | AGGUUUAUCAUUAAGGUCAACCUCUGGAUGUUGUUU | 19263 |
| | gene) | CGGCAUCCUGCAUUGAAUCUGAGUUACUGUCUGUUU | |
| | | UCCUACAAACGGCAGAAGCUGGAGGAGGAAGGGCCU | |
| | | GAGUCCGAGCAGAAGAAAAAGUUCUCCCAUCACAUC | |
| | | AACCGGUGGCGCAUUGCCACGAAGCAGGCCAAUGGG | |
| | | GAGGACAUCGAUGUCAAGGAAACCCGUUUCUUCUGA | |
| | | CGUAAGGGUGCGCAUACGGAAUCUUAUCAGAGUCCG | |
| | | AGCAGAAGAAGAAGUUUCAGAGCUAUGCUGGAAACA | |
| | | GCAUAGCAAGUUGAAAUAAGGCUAGUCCGUUAUCAA | |
| | | CUUGAAAAAGUGGCACCGAGUCGGUGCUUUUUUUUU | |
| | | UUU | |
| Eco3 RT | Coding sequence only. | AUGCGCAUUUACUCUCUGAUCGACAGCCAAACCUUA | SEQ ID NO: |
| | RNA was produced from a | AUGACCAAAGGGUUCGCAUCCGAGGUCAUGAGGAGC | 19264 |
| | CRO and proprietary 5′ cap, | CCAGAACCCCCUAAGAAGUGGGACAUUGCGAAGAAG | |
| | 5′ UTR, 3′ UTR and | AAGGGCGGAAUGCGUACGAUAUACCAUCCCUCUUCU | |
| | encoded poly A of around | AAGGUGAAGCUGAUACAGUACUGGCUGAUGAACAAC | |
| | 120 bases were added. Full | GUGUUCUCCAAAUUGCCGAUGCACAACGCCGCGUAC | |
| | substitution with 1N- | GCUUUCGUGAAGAAUAGAUCUAUCAAGUCUAACGCA | |
| | methyl-pseudouridine | CUGCUGCACGCAGAGAGUAAGAACAAAUACUACGUU | |
| | | AAGAUUGACCUGAAGGACUUCUUUCCAAGCAUCAAG | |
| | | UUCACAGACUUCGAAUAUGCCUUUACCCGGUACCGU | |
| | | GACAGAAUAGAGUUCACGACCGAGUACGACAAAGAA | |
| | | CUGCUUCAGCUGAUUAAGACCAUUUGUUUCAUUUCU | |
| | | GACUCUACACUGCCAAUAGGCUUCCCCACUUCCCCUC | |
| | | UUAUAGCCAAUUUCGUCGCCAGGGAGCUGGACGAGA | |
| | | AGCUCACUCAGAAGCUGAACGCUAUAGACAAGCUCA | |
| | | ACGCUACGUACACUCGCUACGCAGACGACAUAAUCG | |
| | | UGAGCACGAACAUGAAGGGCGCCUCUAAGCUGAUCU | |
| | | UAGACUGCUUCAAGCGGACCAUGAAGGAAAUCGGAC | |
| | | CCGAUUUCAAGAUCAAUAUCAAGAAGUUCAAAAUAU | |
| | | GCUCUGCCAGUGGCGGCUCAAUUGUCGUGACGGGCC | |
| | | UUAAGGUCUGUCAUGACUUCCACAUAACUCUGCACC | |
| | | GGUCUAUGAAGGACAAGAUCCGCCUGCACCUCUCUC | |
| | | UCCUGUCCAAAGGUAUUCUGAAGGACGAGGACCACA | |
| | | ACAAGCUGUCCGGGUACAUCGCCUACGCUAAGGACA | |
| | | UCGAUCCACACUUCUACACCAAGCUCAAUAGGAAGU | |
| | | ACUUCCAGGAGAUCAAGUGGAUACAAAACCUGCAUA | |
| | | AUAAGGUGGAGCCACCAAAGAAGAAAAGAAAGGUCU | |
| | | GA | |
| Eco3 ncRNA- | ncRNA-sgRNA fusion (10 | GAAAUGAUAAGAUUCCGUAAGAGCCAAACCUAGCAU | SEQ ID NO: |
| sgRNA | bp insertion in EMX1 gene) | UUUAUGGGUUAAUAGCCCAUCGGGCCAUGAGUCAUG | 19265 |
| | | GUUUCGCCUAGUAUUUUAGCUAUGCCCGUCGUUCAG | |
| | | UUCGCUGAACAAACGGCAGAAGCUGGAGGAGGAAGG | |
| | | GCCUGAGUCCGAGCAGAAGAAUUACGUCUGCAAAGU | |
| | | UCUCCCAUCACAUCAACCGGUGGCGCAUUGCCACGA | |
| | | AGCAGGCCAAUGGGGAGGACAUCGAUGUCAUCAGCG | |
| | | AACUGAUCGACGUGCUCAAGUAGGUUUGGCUCUUAC | |
| | | GGAAUCUUAUCAGAGUCCGAGCAGAAGAAGAAGUUU | |
| | | CAGAGCUAUGCUGGAAACAGCAUAGCAAGUUGAAAU | |
| | | AAGGCUAGUCCGUUAUCAACUUGAAAAAGUGGCACC | |
| | | GAGUCGGUGCUUUUUUUUUUUU | |
| Eco5 RT | Coding sequence only. | AUGGACGCUACCAGAACGACUCUCCUUGCAUUGGAU | SEQ ID NO: |
| | RNA was produced from a | CUCUUCGGAUCUCCAGGUUGGUCCGCCGAUAAAGAA | 19266 |
| | CRO and proprietary 5′ cap, | AUUCAGAGGCUUCAUGCGCUCAGUAAUCAUGCUGGA | |
| | 5′ UTR, 3′ UTR and | AGGCAUUACAGAAGGAUUAUAUUAAGUAAAAGGCAC | |
| | encoded poly A of around | GGCGGACAGCGUCUUGUGCUUGCACCUGAUUACUUG | |
| | 120 bases were added. Full | UUAAAGACCGUUCAGCGCAACAUUUUGAAGAACGUU | |
| | substitution with 1N- | UUGAGUCAAUUUCCACUGUCACCAUUUGCUACAGCC | |
| | mcthyl-pscudouridinc | UACAGACCGGGAUGCCCAAUCGUGUCUAACGCGCAG | |
| | | CCACACUGCCAACAGCCACAGAUCUUGAAACUCGAU | |
| | | AUAGAAAACUUCUUCGAUUCUAUUAGUUGGUUGCAG | |
| | | GUGUGGCGGGUGUUUCGCCAGGCCCAGUUGCCCCGA | |
| | | AAUGUCGUAACGAUGCUCACUUGGAUAUGUUGUUAU | |
| | | AACGACGCACUUCCGCAGGGUGCCCCUACAUCCCCU | |
| | | GCAAUUUCCAAUCUCGUCAUGAGAAGGUUUGAUGAA | |
| | | CGGAUUGGAGAAUGGUGUCAGGCUCGAGGGAUUACC | |
| | | UACACUCGCUACUGCGAUGACAUGACGUUUAGUGGA | |
| | | CACUUCAAUGCAAGGCAGGUCAAGAAUAAAGUCUGC | |
| | | GGUCUCUUAGCUGAGCUGGGCCUUUCCCUGAAUAAA | |
| | | CGGAAAGGCUGCCUCAUAGCGGCUUGUAAGCGCCAG | |
| | | CAAGUCACCGGCAUUGUUGUGAAUCACAAGCCACAG | |
| | | CUUGCCCGAGAAGCCAGGCGUGCCCUGCGUCAGGAA | |
| | | GUGCACCUGUGCCAGAAAUAUGGAGUUAUCUCUCAU | |
| | | CUCUCACAUAGAGGUGAACUGGAUCCUAGCGGAGAU | |
| | | CUGCACGCUCAGGCGACAGCGUAUCUCUAUGCACUC | |
| | | CAGGGGAGAAUUAACUGGCUUCUUCAAAUUAACCCU | |
| | | GAGGAUGAGGCGUUUCAACAGGCCCGGGAGUCCGUU | |
| | | AAGAGGAUGUUAGUUGCCUGGCCACCAAAGAAGAAA | |
| | | AGAAAGGUCUGA | |
| Eco5 ncRNA- | ncRNA-sgRNA fusion (10 | GAAAUGAUAAGAUUCCGUACGCCAGCAGUGGCAAUA | SEQ ID NO: |
| sgRNA | bp insertion in EMX1 gene) | GCGUUUCCGGCCUUUUGUGCCGGGAGGGUCGGCGAG | 19267 |
| | | UCGCUGACUUAACGCCAGUAGUAUGUCCAUAUACCC | |
| | | AAAGUCGCUUCAUUGUAACAAACGGCAGAAGCUGGA | |
| | | GGAGGAAGGGCCUGAGUCCGAGCAGAAGAAUCCUUC | |
| | | GAGCAAAGUUCUCCCAUCACAUCAACCGGUGGCGCA | |
| | | UUGCCACGAAGCAGGCCAAUGGGGAGGACAUCGAUG | |
| | | UCAUACAGUUACGCGCCUUCGGGAUGGUUUAAUGGU | |
| | | AUUGCCGCUGUUGGCGUACGGAAUCUUAUCAGAGUC | |
| | | CGAGCAGAAGAAGAAGUUUCAGAGCUAUGCUGGAAA | |
| | | CAGCAUAGCAAGUUGAAAUAAGGCUAGUCCGUUAUC | |
| | | AACUUGAAAAAGUGGCACCGAGUCGGUGCUUUUUUU | |
| | | UUUUU | |
| Cas9 | Sequence taken from | GGGUCCCGCAGUCGGCGUCCAGCGGCUCUGCUUGUU | SEQ ID NO: |
| | Intellia patent. All U are N- | CGUGUGUGUGUCGUUGCAGGCCUUAUUCGGAUC | 19268 |
| | 1-methylpseudouridine. | CGCCACCAUGGACAAGAAGUACAGCAUCGGACUGGA | |
| | | CAUCGGAACAAACAGCGUCGGAUGGGCAGUCAU | |
| | | CACAGACGAAUACAAGGUCCCGAGCAAGAAGUUCAA | |
| | | GGUCCUGGGAAACACAGACAGACACAGCAUCAA | |
| | | GAAGAACCUGAUCGGAGCACUGCUGUUCGACAGCGG | |
| | | AGAAACAGCAGAAGCAACAAGACUGAAGAGAAC | |
| | | AGCAAGAAGAAGAUACACAAGAAGAAAGAACAGAAU | |
| | | CUGCUACCUGCAGGAAAUCUUCAGCAACGAAAU | |
| | | GGCAAAGGUCGACGACAGCUUCUUCCACAGACUGGA | |
| | | AGAAAGCUUCCUGGUCGAAGAAGACAAGAAGCA | |
| | | CGAAAGACACCCGAUCUUCGGAAACAUCGUCGACGA | |
| | | AGUCGCAUACCACGAAAAGUACCCGACAAUCUAC | |
| | | CACCUGAGAAAGAAGCUGGUCGACAGCACAGACAAG | |
| | | GCAGACCUGAGACUGAUCUACCUGGCACUGGCA | |
| | | CACAUGAUCAAGUUCAGAGGACACCUCCUGAUCGAA | |
| | | GGAGACCUGAACCCGGACAACAGCGACGUCGAC | |
| | | AAGCUGUUCAUCCAGCUGGUCCAGACAUACAACCAG | |
| | | CUGUUCGAAGAAAACCCGAUCAACGCAAGCGGA | |
| | | GUCGACGCAAAGGCAAUCCUGAGCGCAAGACUGAGC | |
| | | AAGAGCAGAAGACUGGAAAACCUGAUCGCACAG | |
| | | CUGCCGGGAGAAAAGAAGAACGGACUGUUCGGAAAC | |
| | | CUGAUCGCACUGAGCCUGGGACUGACACCGAAC | |
| | | UUCAAGAGCAACUUCGACCUGGCAGAAGACGCAAAG | |
| | | CUGCAGCUGAGCAAGGACACAUACGACGACGAC | |
| | | CUGGACAACCUGCUGGCACAGAUCGGAGACCAGUAC | |
| | | GCAGACCUGUUCCUGGCAGCAAAGAACCUGAGC | |
| | | GACGCAAUCCUGCUGAGCGACAUCCUGAGAGUCAAC | |
| | | ACAGAAAUCACAAAGGCACCGCUGAGCGCAAGC | |
| | | AUGAUCAAGAGAUACGACGAACACCACCAGGACCUG | |
| | | ACACUGCUGAAGGCACUGGUCAGACAGCAGCUG | |
| | | CCGGAAAAGUACAAGGAAAUCUUCUUCGACCAGAGC | |
| | | AAGAACGGAUACGCAGGAUACAUCGACGGAGGA | |
| | | GCAAGCCAGGAAGAAUUCUACAAGUUCAUCAAGCCG | |
| | | AUCCUGGAAAAGAUGGACGGAACAGAAGAACUG | |
| | | CUGGUCAAGCUGAACAGAGAAGACCUGCUGAGAAAG | |
| | | CAGAGAACAUUCGACAACGGAAGCAUCCCGCAC | |
| | | CAGAUCCACCUGGGAGAACUGCACGCAAUCCUGAGA | |
| | | AGACAGGAAGACUUCUACCCGUUCCUGAAGGAC | |
| | | AACAGAGAAAAGAUCGAAAAGAUCCUGACAUUCAGA | |
| | | AUCCCGUACUACGUCGGACCGCUGGCAAGAGGA | |
| | | AACAGCAGAUUCGCAUGGAUGACAAGAAAGAGCGAA | |
| | | GAAACAAUCACACCGUGGAACUUCGAAGAAGUC | |
| | | GUCGACAAGGGAGCAAGCGCACAGAGCUUCAUCGAA | |
| | | AGAAUGACAAACUUCGACAAGAACCUGCCGAAC | |
| | | GAAAAGGUCCUGCCGAAGCACAGCCUGCUGUACGAA | |
| | | UACUUCACAGUCUACAACGAACUGACAAAGGUC | |
| | | AAGUACGUCACAGAAGGAAUGAGAAAGCCGGCAUUC | |
| | | CUGAGCGGAGAACAGAAGAAGGCAAUCGUCGAC | |
| | | CUGCUGUUCAAGACAAACAGAAAGGUCACAGUCAAG | |
| | | CAGCUGAAGGAAGACUACUUCAAGAAGAUCGAA | |
| | | UGCUUCGACAGCGUCGAAAUCAGCGGAGUCGAAGAC | |
| | | AGAUUCAACGCAAGCCUGGGAACAUACCACGAC | |
| | | CUGCUGAAGAUCAUCAAGGACAAGGACUUCCUGGAC | |
| | | AACGAAGAAAACGAAGACAUCCUGGAAGACAUC | |
| | | GUCCUGACACUGACACUGUUCGAAGACAGAGAAAUG | |
| | | AUCGAAGAAAGACUGAAGACAUACGCACACCUG | |
| | | UUCGACGACAAGGUCAUGAAGCAGCUGAAGAGAAGA | |
| | | AGAUACACAGGAUGGGGAAGACUGAGCAGAAAG | |
| | | CUGAUCAACGGAAUCAGAGACAAGCAGAGCGGAAAG | |
| | | ACAAUCCUGGACUUCCUGAAGAGCGACGGAUUC | |
| | | GCAAACAGAAACUUCAUGCAGCUGAUCCACGACGAC | |
| | | AGCCUGACAUUCAAGGAAGACAUCCAGAAGGCA | |
| | | CAGGUCAGCGGACAGGGAGACAGCCUGCACGAACAC | |
| | | AUCGCAAACCUGGCAGGAAGCCCGGCAAUCAAG | |
| | | AAGGGAAUCCUGCAGACAGUCAAGGUCGUCGACGAA | |
| | | CUGGUCAAGGUCAUGGGAAGACACAAGCCGGAA | |
| | | AACAUCGUCAUCGAAAUGGCAAGAGAAAACCAGACA | |
| | | ACACAGAAGGGACAGAAGAACAGCAGAGAAAGAAUG | |
| | | AAGAGAAUCGAAGAAGGAAUCAAGGAACUGGGAAGC | |
| | | CAGAUCCUGAAGGAACACCCGGUCGAAAAC | |
| | | ACACAGCUGCAGAACGAAAAGCUGUACCUGUACUAC | |
| | | CUGCAGAACGGAAGAGACAUGUACGUCGACCAG | |
| | | GAACUGGACAUCAACAGACUGAGCGACUACGACGUC | |
| | | GACCACAUCGUCCCGCAGAGCUUCCUGAAGGACG | |
| | | ACAGCAUCGACAACAAGGUCCUGACAAGAAGCGACA | |
| | | AGAACAGAGGAAAGAGCGACAACGUCCCGAGCG | |
| | | AAGAAGUCGUCAAGAAGAUGAAGAACUACUGGAGAC | |
| | | AGCUGCUGAACGCAAAGCUGAUCACACAGAGAA | |
| | | AGUUCGACAACCUGACAAAGGCAGAGAGAGGAGGAC | |
| | | UGAGCGAACUGGACAAGGCAGGAUUCAUCAAGA | |
| | | GACAGCUGGUCGAAACAAGACAGAUCACAAAGCACG | |
| | | UCGCACAGAUCCUGGACAGCAGAAUGAACACAA | |
| | | AGUACGACGAAAACGACAAGCUGAUCAGAGAAGUCA | |
| | | AGGUCAUCACACUGAAGAGCAAGCUGGUCAGCG | |
| | | ACUUCAGAAAGGACUUCCAGUUCUACAAGGUCAGAG | |
| | | AAAUCAACAACUACCACCACGCACACGACGCAU | |
| | | ACCUGAACGCAGUCGUCGGAACAGCACUGAUCAAGA | |
| | | AGUACCCGAAGCUGGAAAGCGAAUUCGUCUACG | |
| | | GAGACUACAAGGUCUACGACGUCAGAAAGAUGAUCG | |
| | | CAAAGAGCGAACAGGAAAUCGGAAAGGCAACAG | |
| | | CAAAGUACUUCUUCUACAGCAACAUCAUGAACUUCU | |
| | | UCAAGACAGAAAUCACACUGGCAAACGGAGAAA | |
| | | UCAGAAAGAGACCGCUGAUCGAAACAAACGGAGAAA | |
| | | CAGGAGAAAUCGUCUGGGACAAGGGAAGAGACU | |
| | | UCGCAACAGUCAGAAAGGUCCUGAGCAUGCCGCAGG | |
| | | UCAACAUCGUCAAGAAGACAGAAGUCCAGACAG | |
| | | GAGGAUUCAGCAAGGAAAGCAUCCUGCCGAAGAGAA | |
| | | ACAGCGACAAGCUGAUCGCAAGAAAGAAGGACU | |
| | | GGGACCCGAAGAAGUACGGAGGAUUCGACAGCCCGA | |
| | | CAGUCGCAUACAGCGUCCUGGUCGUCGCAAAGG | |
| | | UCGAAAAGGGAAAGAGCAAGAAGCUGAAGAGCGUCA | |
| | | AGGAACUGCUGGGAAUCACAAUCAUGGAAAGAA | |
| | | GCAGCUUCGAAAAGAACCCGAUCGACUUCCUGGAAG | |
| | | CAAAGGGAUACAAGGAAGUCAAGAAGGACCUGA | |
| | | UCAUCAAGCUGCCGAAGUACAGCCUGUUCGAACUGG | |
| | | AAAACGGAAGAAAGAGAAUGCUGGCAAGCGCAG | |
| | | GAGAACUGCAGAAGGGAAACGAACUGGCACUGCCGA | |
| | | GCAAGUACGUCAACUUCCUGUACCUGGCAAGCC | |
| | | ACUACGAAAAGCUGAAGGGAAGCCCGGAAGACAACG | |
| | | AACAGAAGCAGCUGUUCGUCGAACAGCACAAGC | |
| | | ACUACCUGGACGAAAUCAUCGAACAGAUCAGCGAAU | |
| | | UCAGCAAGAGAGUCAUCCUGGCAGACGCAAACC | |
| | | UGGACAAGGUCCUGAGCGCAUACAACAAGCACAGAG | |
| | | ACAAGCCGAUCAGAGAACAGGCAGAAAACAUCA | |
| | | UCCACCUGUUCACACUGACAAACCUGGGAGCACCGG | |
| | | CAGCAUUCAAGUACUUCGACACAACAAUCGACAG | |
| | | AAAGAGAUACACAAGCACAAAGGAAGUCCUGGACGC | |
| | | AACACUGAUCCACCAGAGCAUCACAGGACUGUA | |
| | | CGAAACAAGAAUCGACCUGAGCCAGCUGGGAGGAGA | |
| | | CGGAGGAGGAAGCCCGAAGAAGAAGAGAAAGGU | |
| | | CUAGCUAGCCAUCACAUUUAAAAGCAUCUCAGCCUA | |
| | | CCAUGAGAAUAAGAGAAAGAAAAUGAAGAUCAA | |
| | | UAGCUUAUUCAUCUCUUUUUCUUUUUCGUUGGUGUA | |
| | | AAGCCAACACCCUGUCUAAAAAACAUAAAUUUC | |
| | | UUUAAUCAUUUUGCCUCUUUUCUCUGUGCUUCAAUU | |
| | | AAUAAAAAAUGGAAAGAACCUCGAGAAAAAAAA | |
| | | AAAAAAAAAAAAAAAAAAAAAAAAAAAAAAAAAAA | |
| | | AAAAAAAAAAAAAAAAAAAAAAAAAAAAAAAAA | |
| | | AAAAAAAAAAAAAAUCUAG | |
| EMX1 sgRNA | Contains chemical | GAGUCCGAGCAGAAGAAGAAGUUUCAGAGCUAUGCU | SEQ ID NO: |
| modified | modifications from | GGAAACAGCAUAGCAAGUUGAAAUAAGGCUAGUCCG | 19269 |
| | Synthego: 2′-F, 2′-O- | UUAUCAACUUGAAAAAGUGGCACCGAGUCGGUGCUU | |
| | methyl, phosphorothioates | UUU | |
| EMX1 sgRNA | No modifications | GAGUCCGAGCAGAAGAAGAAGUUUCAGAGCUAUGCU | SEQ ID NO: |
| unmodified | | GGAAACAGCAUAGCAAGUUGAAAUAAGGCUAGUCCG | 19270 |
| | | UUAUCAACUUGAAAAAGUGGCACCGAGUCGGUGCUU | |
| | | UUU | |
| Eco3 ncRNA | Eco3 ncRNA only with 10 | GGGAUAAUUGAUAAGAUUCCGUAAGAGCCAAACCUA | SEQ ID NO: |
| | bp insertion | GCAUUUUAUGGGUUAAUAGCCCAUCGGGCCAUGAGU | 19271 |
| | | CAUGGUUUCGCCUAGUAUUUUAGCUAUGCCCGUCGU | |
| | | UCAGUUCGCUGAACAAACGGCAGAAGCUGGAGGAGG | |
| | | AAGGGCCUGAGUCCGAGCAGAAGAAUUACGUCUGCA | |
| | | AAGUUCUCCCAUCACAUCAACCGGUGGCGCAUUGCC | |
| | | ACGAAGCAGGCCAAUGGGGAGGACAUCGAUGUCAUC | |
| | | AGCGAACUGAUCGACGUGCUCAAGUAGGUUUGGCUC | |
| | | UUACGGAAUCUUAUCAGAU | |
| Eco3 ncRNA- | Eco3 ncRNA only with 10 | GGGAUAAUUGAUAAGAUUCCGUAAGAGCCAAACCUA | SEQ ID NO: |
| MS2 | bp insertion and 3′ MS2 | GCAUUUUAUGGGUUAAUAGCCCAUCGGGCCAUGAGU | 19272 |
| | stem loop | CAUGGUUUCGCCUAGUAUUUUAGCUAUGCCCGUCGU | |
| | | UCAGUUCGCUGAACAAACGGCAGAAGCUGGAGGAGG | |
| | | AAGGGCCUGAGUCCGAGCAGAAGAAUUACGUCUGCA | |
| | | AAGUUCUCCCAUCACAUCAACCGGUGGCGCAUUGCC | |
| | | ACGAAGCAGGCCAAUGGGGAGGACAUCGAUGUCAUC | |
| | | AGCGAACUGAUCGACGUGCUCAAGUAGGUUUGGCUC | |
| | | UUACGGAAUCUUAUCAACAUGAGGAUCACCCAUGUG | |
| | | AU | |
| Eco3 cicular | Eco3 ncRNA only wih | UACCGGCGAAACAAAAGAAAAAACCAAAAAAACAAA | SEQ ID NO: |
| ncRNA | 10 bp insertion in circular | ACACAUGAUAAGAUUCCGUAAGAGCCAAACCUAGCA | 19273 |
| | format | UUUUAUGGGUUAAUAGCCCAUCGGGCCAUGAGUCAU | |
| | | GGUUUCGCCUAGUAUUUUAGCUAUGCCCGUCGUUCA | |
| | | GUUCGCUGAACAAACGGCAGAAGCUGGAGGAGGAAG | |
| | | GGCCUGAGUCCGAGCAGAAGAAUUACGUCUGCAAAG | |
| | | UUCUCCCAUCACAUCAACCGGUGGCGCAUUGCCACG | |
| | | AAGCAGGCCAAUGGGGAGGACAUCGAUGUCAUCAGC | |
| | | GAACUGAUCGACGUGCUCAAGUAGGUUUGGCUCUUA | |
| | | CGGAAUCUUAUCAAAAAAACAAAAAACAAAACGGCU | |
| | | AUUAUGCGUUACCGGCG | |
| Aco1 RT | Aco1 RT RNA sequence | AGGGGGAAAUAAGAGAGAAAAGAAGAGUAAGAAGA | SEQ ID NO: |
| | including 5′ and 3′ UTRs | AAUAUAAGAGCCACCAUGGAGCCCAAUGACUACGUA | 19274 |
| | | AAUAGGUUGCGACAUGCUAUGGAAAUAAGUGAAAAC | |
| | | CCGCGCUUUAGCCCUGAGUACAUUGCUCAAUGUUGU | |
| | | ACUUACGCCGAGAAUCUCCUCAAGCAAGGGCUGCCA | |
| | | GUUCUGUUCGAUCAAACGCAUAUUCGGAAAGUUCUG | |
| | | GGGAUGGCGGCACCUCGAUUGUGUGAUUAUCACAGA | |
| | | UUCACAAUCCCAAAGCACAACGGAUCUAGAAUUAUC | |
| | | ACGGCCCCAUCUAGGAAGCUGAAGCUUCGACAACAA | |
| | | UGGAUAUACCAGAAUAUCCUUAUACGAAAGGAGGCU | |
| | | UCACCGUACACGCACGGAUUUGUUCCUGAACGCAGC | |
| | | AUCGUGACUAACGCAAUCCUCCAUAUAGGAUACGCA | |
| | | UACACCUACUGCGUGGAUAUCACGGAUUUCUUUCCU | |
| | | AGCAUCACUAAGAAGCAGGUCUUGCCUAUAUUCCGA | |
| | | AAUAUGGGCUAUAGUGGUUCUGCUGCAAAUACUCUC | |
| | | UGCGACCUCUGUUGCUAUGACGGGGUCCUCCCCCAG | |
| | | GGGGCGCCUACUAGCCCAUACCUCAGUAACAUGAUU | |
| | | UGUCGCGAUCUUGAUGACGAAUUGGGGGCUAUGGCG | |
| | | CGGCGGUUCCGGGGGAUUUUUACACGGUAUGCGGAU | |
| | | GACAUAGCUAUCUCCACAAACCAACAACAGCCGCAA | |
| | | CUUUUGGAUGCCUUGGGACUUAUCCUCGGGAAGCAC | |
| | | GGAUUUCUUAUGAAUCUCGAUAAGUGUCGAGUCUAU | |
| | | AAUCCUGGACAGCCCAAAAGAAUUACUGGAUUGACC | |
| | | GUUCACAAUAGAGUAUCAGUUCCGAAAACCUUUAAG | |
| | | CGGAAAUUGCGGCAGGAAAUACAUUACUGUCAGAAG | |
| | | UUCGGAGUGACUGCACAUUUGGAAAACACGAAGGCU | |
| | | GCACGAUCCAUCCACUAUAGGGAACAUCUGUAUGGA | |
| | | AAGGCAUACUAUGUUAAAAUGGUUGAGCCUGAGCUC | |
| | | GGGGCGCACUUCCUCGAUGAGUUGUCAAAGGUAGAC | |
| | | UGGCCAGAGCCACCAAAGAAGAAAAGAAAGGUCUGA | |
| | | UAAUAGGCUGGAGCCUCGGUGGCCAUGCUUCUUGCC | |
| | | CCUUGGGCCUCCCCCCAGCCCCUCCUCCCCUUCCUGC | |
| | | ACCCGUACCCCCGUGGUCUUUGAAUAAAGUCUGAGU | |
| | | GGGCGGCCCUGCA | |
| Aco1 | Aco1 ncRNA containing 12 | guauaaaaccgggaacgaucagaccggggugaauucgcccccuugaucaaacggc | SEQ ID NO: |
| ncRNA_12 | bp insertion in EMX1 gene | acuaaccacuguuugccguggugcguCAGAAGAAGAAGGGCUCC | 19275 |
| | | CAUCACAUCAACCGGUGGCGCAUUGCCACGAAGCAG | |
| | | GCCAAUGGGGAGGACAUCGAUGUCACCUCCAAUGuuc | |
| | | gaacgaucgGGUGGGCAACCACAAACCCACGAGGGCAGA | |
| | | GUGCUGCUUGCUGCUacgcacgcacggcaaacagacagauccauuau | |
| | | uauuacaauuuauuuagugaucguucccgguuuuauacAAUAACAACG | |
| | | UCACCUCCAAUGACUAGGGguuucagagcuaugcuggaaacagca | |
| | | uagcaaguugaaauaaggcuaguccguuaucaacuugaaaaaguggcaccgagu | |
| | | cggugcuuuuu | |
| Aco1 ncRNA- | Aco1 ncRNA-sgRNA with | GGGGUAUAAAACCGGGAACGAUCAGACCGGGGUGAA | SEQ ID NO: |
| sgRNA_50 | 50 bp insertion | UUCGCCCCCUUGAUCAAACGGCACUAACCACUGUUU | 19276 |
| | | GCCGUGCGUGCGUACAAACGGCAGAAGCUGGAGGAG | |
| | | GAAGGGCCUGAGUCCGAGCAGAAGAAAACUCGAGCU | |
| | | CUGAUCAGUAUCAUGGGGCCCAUGGUUGAAUGACUC | |
| | | CUAUAAAGUUCUCCCAUCACAUCAACCGGUGGCGCA | |
| | | UUGCCACGAAGCAGGCCAAUGGGGAGGACAUCGAUG | |
| | | UCAACGCACGCACGGCAAACAGACAGAUCCAUUAUU | |
| | | AUUACAAUUUAUUUAGUGAUCGUUCCCGGUUUUAUA | |
| | | CGAGUCCGAGCAGAAGAAGAAGUUUCAGAGCUAUGC | |
| | | UGGAAACAGCAUAGCAAGUUGAAAUAAGGCUAGUCC | |
| | | GUUAUCAACUUGAAAAAGUGGCACCGAGUCGGUGCU | |
| | | UUUUCCUGCA | |
| Aco1 sgRNA- | Aco1 sgRNA-ncRNA with | GGGGAGUCCGAGCAGAAGAAGAAGUUUCAGAGCUAU | SEQ ID NO: |
| ncRNA_50 | 50 bp insertion | GCUGGAAACAGCAUAGCAAGUUGAAAUAAGGCUAGU | 19277 |
| | | CCGUUAUCAACUUGAAAAAGUGGCACCGAGUCGGUG | |
| | | CUUUUUGUAUAAAACCGGGAACGAUCAGACCGGGGU | |
| | | GAAUUCGCCCCCUUGAUCAAACGGCACUAACCACUG | |
| | | UUUGCCGUGCGUGCGUACAAACGGCAGAAGCUGGAG | |
| | | GAGGAAGGGCCUGAGUCCGAGCAGAAGAAAACUCGA | |
| | | GCUCUGAUCAGUAUCAUGGGGCCCAUGGUUGAAUGA | |
| | | CUCCUAUAAAGUUCUCCCAUCACAUCAACCGGUGGC | |
| | | GCAUUGCCACGAAGCAGGCCAAUGGGGAGGACAUCG | |
| | | AUGUCAACGCACGCACGGCAAACAGACAGAUCCAUU | |
| | | AUUAUUACAAUUUAUUUAGUGAUCGUUCCCGGUUUU | |
| | | AUACCCUGCA | |
| Eco3_EMX1 | Eco3 ncRNA containing 25 | GAGUCCGAGCAGAAGAAGAAGUUUCAGAGCUAUGCU | SEQ ID NO: |
| gRNA_ncRNA_ | bp insertion in EMX1 gene | GGAAACAGCAUAGCAAGUUGAAAUAAGGCUAGUCCG | 19278 |
| 25 | fused to EMX1 sgRNA at | UUAUCAACUUGAAAAAGUGGCACCGAGUCGGUGCUU | |
| | 5′ end | UUUAAUAACAACGAAAUGAUAAGAUUCCGUAAGAGC | |
| | | CAAACCUAGCAUUUUAUGGGUUAAUAGCCCAUCGGG | |
| | | CCAUGAGUCAUGGUUUCGCCUAGUAUUUUAGCUAUG | |
| | | CCCGUCGUUCAGUUCGCUGAACAAACGGCAGAAGCU | |
| | | GGAGGAGGAAGGGCCUGAGUCCGAGCAGAAGAAaAC | |
| | | UCGAGCUCUGAAUGACUCCUAUAaaguuCUCCCAUCAC | |
| | | AUCAACCGGUGGCGCAUUGCCACGAAGCAGGCCAAU | |
| | | GGGGAGGACAUCGAUGUCAUCAGCGAACUGAUCGAC | |
| | | GUGCUCAAGUAGGUUUGGCUCUUACGGAAUCUUAUC | |
| | | A | |
| Eco3_ncRNA | Eco3 ncRNA containing 25 | GAAAUGAUAAGAUUCCGUAAGAGCCAAACCUAGCAU | SEQ ID NO: |
| only | bp insertion in EMX1 gene | UUUAUGGGUUAAUAGCCCAUCGGGCCAUGAGUCAUG | 19279 |
| | | GUUUCGCCUAGUAUUUUAGCUAUGCCCGUCGUUCAG | |
| | | UUCGCUGAACAAACGGCAGAAGCUGGAGGAGGAAGG | |
| | | GCCUGAGUCCGAGCAGAAGAAUUACGUCUGCAAAGU | |
| | | UCUCCCAUCACAUCAACCGGUGGCGCAUUGCCACGA | |
| | | AGCAGGCCAAUGGGGAGGACAUCGAUGUCAUCAGCG | |
| | | AACUGAUCGACGUGCUCAAGUAGGUUUGGCUCUUAC | |
| | | GGAAUCUUAUCA | |
| Eco3_ncRNA_ | Eco3 ncRNA containing 25 | GAAAUGAUAAGAUUCCGUAAGAGCCAAACCUAGCAU | SEQ ID NO: |
| AAVS1 | bp insertion in AAVS1 | UUUAUGGGUUAAUAGCCCAUCGGGCCAUGAGUCAUG | 19280 |
| gRNA_25 | gene fused to AAVS1 | GUUUCGCCUAGUAUUUUAGCUAUGCCCGUCGUUCAG | |
| | sgRNA at 3′ end | UUCGCUGACUGUCCCCUCCACCCCACAGUGGGGCCAC | |
| | | UAGGGACAGAAAUGACAGUGGUUGGUGCUCUAAAAA | |
| | | UUAAUGACAGAAAAGCCCCAUCCUUAGGCCUCCUCC | |
| | | UUCCUAGUCUCCUGAUAUUGGGUCUAACCCCCAUCA | |
| | | GCGAACUGAUCGACGUGCUCAAGUAGGUUUGGCUCU | |
| | | UACGGAAUCUUAUCAAAUAACAACGGGGCCACUAGG | |
| | | GACAGGAUGUUUCAGAGCUAUGCUGGAAACAGCAUA | |
| | | GCAAGUUGAAAUAAGGCUAGUCCGUUAUCAACUUGA | |
| | | AAAAGUGGCACCGAGUCGGUGCUUUUU | |
| Eco3_ncRNA_ | Eco3 ncRNA containing 25 | GAAAUGAUAAGAUUCCGUAAGAGCCAAACCUAGCAU | SEQ ID NO: |
| EMX1 | bp insertion in EMX1 gene | UUUAUGGGUUAAUAGCCCAUCGGGCCAUGAGUCAUG | 19281 |
| gRNA_25 | fused to EMX1 sgRNA at | GUUUCGCCUAGUAUUUUAGCUAUGCCCGUCGUUCAG | |
| | 3′ end | UUCGCUGAACAAACGGCAGAAGCUGGAGGAGGAAGG | |
| | | GCCUGAGUCCGAGCAGAAGAAaACUCGAGCUCUGAA | |
| | | UGACUCCUAUAaaguuCUCCCAUCACAUCAACCGGUGG | |
| | | CGCAUUGCCACGAAGCAGGCCAAUGGGGAGGACAUC | |
| | | GAUGUCAUCAGCGAACUGAUCGACGUGCUCAAGUAG | |
| | | GUUUGGCUCUUACGGAAUCUUAUCAAAUAACAACGA | |
| | | GUCCGAGCAGAAGAAGAAGUUUCAGAGCUAUGCUGG | |
| | | AAACAGCAUAGCAAGUUGAAAUAAGGCUAGUCCGUU | |
| | | AUCAACUUGAAAAAGUGGCACCGAGUCGGUGCUUUU | |
| | | U | |
| Eco3_ncRNA_ | Eco3 ncRNA containing 50 | GAAAUGAUAAGAUUCCGUAAGAGCCAAACCUAGCAU | SEQ ID NO: |
| EMXI | bp insertion in EMXI gene | UUUAUGGGUUAAUAGCCCAUCGGGCCAUGAGUCAUG | 19282 |
| gRNA_50 | fused to EMX1 sgRNA at | GUUUCGCCUAGUAUUUUAGCUAUGCCCGUCGUUCAG | |
| | 3′ end | UUCGCUGAACAAACGGCAGAAGCUGGAGGAGGAAGG | |
| | | GCCUGAGUCCGAGCAGAAGAAaACUCGAGCUCUGAU | |
| | | CAGUAUCAUGGGGCCCAUGGUUGAAUGACUCCUAUA | |
| | | aaguuCUCCCAUCACAUCAACCGGUGGCGCAUUGCCAC | |
| | | GAAGCAGGCCAAUGGGGAGGACAUCGAUGUCAUCAG | |
| | | CGAACUGAUCGACGUGCUCAAGUAGGUUUGGCUCUU | |
| | | ACGGAAUCUUAUCAAAUAACAACGAGUCCGAGCAGA | |
| | | AGAAGAAGUUUCAGAGCUAUGCUGGAAACAGCAUAG | |
| | | CAAGUUGAAAUAAGGCUAGUCCGUUAUCAACUUGAA | |
| | | AAAGUGGCACCGAGUCGGUGCUUUUU | |
| Eco3_ncRNA_ | Eco3 ncRNA containing 75 | GAAAUGAUAAGAUUCCGUAAGAGCCAAACCUAGCAU | SEQ ID NO: |
| EMX1 | bp insertion in EMX1 gene | UUUAUGGGUUAAUAGCCCAUCGGGCCAUGAGUCAUG | 19283 |
| gRNA_75 | fused to EMX1 sgRNA at | GUUUCGCCUAGUAUUUUAGCUAUGCCCGUCGUUCAG | |
| | 3′ end | UUCGCUGAACAAACGGCAGAAGCUGGAGGAGGAAGG | |
| | | GCCUGAGUCCGAGCAGAAGAAaACUCGAGCUCUGAG | |
| | | CCCCACCGCAGCUUGCCAGCACUUUCAGUAUCAUGG | |
| | | GGCCCAUGGUUGAAUGACUCCUAUAaaguuCUCCCAUC | |
| | | ACAUCAACCGGUGGCGCAUUGCCACGAAGCAGGCCA | |
| | | AUGGGGAGGACAUCGAUGUCAUCAGCGAACUGAUCG | |
| | | ACGUGCUCAAGUAGGUUUGGCUCUUACGGAAUCUUA | |
| | | UCAAAUAACAACGAGUCCGAGCAGAAGAAGAAGUUU | |
| | | CAGAGCUAUGCUGGAAACAGCAUAGCAAGUUGAAAU | |
| | | AAGGCUAGUCCGUUAUCAACUUGAAAAAGUGGCACC | |
| | | GAGUCGGUGCUUUUU | |
| Eco3_ncRNA_ | Eco3 ncRNA containing | GAAAUGAUAAGAUUCCGUAAGAGCCAAACCUAGCAU | SEQ ID NO: |
| EMX1 | 100 bp insertion in EMX1 | UUUAUGGGUUAAUAGCCCAUCGGGCCAUGAGUCAUG | 19284 |
| gRNA_100 | gene fused to EMX1 | GUUUCGCCUAGUAUUUUAGCUAUGCCCGUCGUUCAG | |
| | sgRNA at 3′ end | UUCGCUGAACAAACGGCAGAAGCUGGAGGAGGAAGG | |
| | | GCCUGAGUCCGAGCAGAAGAAaACUCGAGCUCUGAG | |
| | | CCCCACUGUCGAGAAGUAUGUAUCUCGCUCCCGCAG | |
| | | CUUGCCAGCACUUUCAGUAUCAUGGGGCCCAUGGUU | |
| | | GAAUGACUCCUAUAaaguuCUCCCAUCACAUCAACCGG | |
| | | UGGCGCAUUGCCACGAAGCAGGCCAAUGGGGAGGAC | |
| | | AUCGAUGUCAUCAGCGAACUGAUCGACGUGCUCAAG | |
| | | UAGGUUUGGCUCUUACGGAAUCUUAUCAAAUAACAA | |
| | | CGAGUCCGAGCAGAAGAAGAAGUUUCAGAGCUAUGC | |
| | | UGGAAACAGCAUAGCAAGUUGAAAUAAGGCUAGUCC | |
| | | GUUAUCAACUUGAAAAAGUGGCACCGAGUCGGUGCU | |
| | | UUUU | |
| Eco3_ncRNA_ | Eco3 ncRNA containing | GAAAUGAUAAGAUUCCGUAAGAGCCAAACCUAGCAU | SEQ ID NO: |
| EMX1 | GFP gene insertion in | UUUAUGGGUUAAUAGCCCAUCGGGCCAUGAGUCAUG | 19285 |
| gRNA_GFP | EMX1 gene fused to EMX1 | GUUUCGCCUAGUAUUUUAGCUAUGCCCGUCGUUCAG | |
| gene | sgRNA at 3′ end. Entire | UUCGCUGAACAAACGGCAGAAGCUGGAGGAGGAAGG | |
| | GFP cassette is in antisense | GCCUGAGUCCGAGCAGAAGAAcccauauguccuuccgagugaga | |
| | orientation and contains | gacacaaaaaauuccaacacacuauugcaaugaaauaaauuuccuuuauuagcc | |
| | mini EF1a promoter and | agaagucagaugcucaaggggcuucaugauguccccauaauuuuuggcagaggg | |
| | beta globin polyA signal. | aaaaagaucucagugguauuugugagccagggcauuggccacaccagccaccacc | |
| | | uucugauaggcagccugcaccugaggagugcggccgcuuuacuuguacagcucg | |
| | | uccaugccgagagugaucccggcggcggucacgaacuccagcaggaccauguga | |
| | | ucgcgcuucucguuggggucuuugcucagggcggacugggugcucagguagug | |
| | | guugucgggcagcagcacggggccgucgccgauggggguguucugcugguagu | |
| | | ggucggcgagcugcacgcugccguccucgauguuguggggaucuugaaguuc | |
| | | accuugaugccguucuucugcuugucggccaugauauagacguuguggcuguu | |
| | | guaguuguacuccagcuugugccccaggauguugccguccuccuugaagucgau | |
| | | gcccuucagcucgaugegguucaccagggugucgcccucgaacuucaccucggc | |
| | | gcgggucuuguaguugccgucguccuugaagaagaugguggcuccuggacgu | |
| | | agccuucgggcauggcggacuugaagaagucgugcugcuucauguggucgggg | |
| | | uagcggcugaagcacugcacgccguaggucaggguggucacgagggugggccag | |
| | | ggcacgggcagcuugccgguggugcagaugaacuucagggucagcuugccguag | |
| | | guggcaucgcccucgcccucgccggacacgcugaacuuguggccguuuacgucg | |
| | | ccguccagcucgaccaggaugggcaccaccccggugaacagcuccucgcccuugc | |
| | | ucaccaugguggcgaccgguggaucccgggccegcgguaccgucgacugcagaa | |
| | | uucgaagcuugagcucgagaucugaguccgguagegcuageggaucugacgguu | |
| | | cacuaaacccuguguucuggeggcaaacccguugcgaaaaagaacguucacggcg | |
| | | acuacugcacuuauauacgguucucccccacccucgggaaaaaggeggagccagu | |
| | | acacgacaucacuuucccaguuuaccccgcgccaccuucucuaggcaccgguuca | |
| | | auugccgaccccuccccccaacuucucggggacuguggggaugugegcucugc | |
| | | ccguuCUCCCAUCACAUCAACCGGUGGCGCAUUGCCAC | |
| | | GAAGCAGGCCAAUGGGGAGGACAUCGAUGUCAUCAG | |
| | | CGAACUGAUCGACGUGCUCAAGUAGGUUUGGCUCUU | |
| | | ACGGAAUCUUAUCAAAUAACAACGAGUCCGAGCAGA | |
| | | AGAAGAAGUUUCAGAGCUAUGCUGGAAACAGCAUAG | |
| | | CAAGUUGAAAUAAGGCUAGUCCGUUAUCAACUUGAA | |
| | | AAAGUGGCACCGAGUCGGUGCUUUUU | |
| Eco3_NT_ | Eco3 ncRNA containing 25 | GAAAUGAUAAGAUUCCGUAAGAGCCAAACCUAGCAU | SEQ ID NO: |
| ncRNA_EMX | bp insertion (on opposite | UUUAUGGGUUAAUAGCCCAUCGGGCCAUGAGUCAUG | 19286 |
| gRNA_25 | strand of sgRNA cut) in | GUUUCGCCUAGUAUUUUAGCUAUGCCCGUCGUUCAG | |
| | EMX1 gene fused to EMX1 | UUCGCUGAugacaucgauguccuccccauuggccugcuucguggcaaugc | |
| | sgRNA at 3′ end | gccaccgguugaugugaugggagaacuuUAUAGGAGUCAUUCAGA | |
| | | GCUCGAGUuuucuucugcucggacucaggcccuuccuccuccagcuucug | |
| | | ccguuuguUCAGCGAACUGAUCGACGUGCUCAAGUAGG | |
| | | UUUGGCUCUUACGGAAUCUUAUCAAAUAACAACGAG | |
| | | UCCGAGCAGAAGAAGAAGUUUCAGAGCUAUGCUGGA | |
| | | AACAGCAUAGCAAGUUGAAAUAAGGCUAGUCCGUUA | |
| | | UCAACUUGAAAAAGUGGCACCGAGUCGGUGCUUUUU | |
| Aco1_EMX1 | Aco1 ncRNA containing 25 | gaguccgagcagaagaagaaguuucagagcuaugcuggaaacagcauagcaagu | SEQ ID NO: |
| gRNA_ncRNA_ | bp insertion in EMX1 gene | ugaaauaaggcuaguccguuaucaacuugaaaaaguggcaccgagucggugcuu | 19287 |
| 25 | fused to EMX1 sgRNA at | uuuAAUAACAACguauaaaaccgggaacgaucagaccggggugaauucgc | |
| | 5′ end | ccccuugaucaaacggcacuaaccacuguuugccgugcgugcguacaaacggcag | |
| | | aagcuggaggaggaagggccugaguccgagcagaagaaaACUCGAGCUC | |
| | | UGAAUGACUCCUAUAaaguucucccaucacaucaaccgguggcgcau | |
| | | ugccacgaagcaggccaauggggaggacaucgaugucaacgcacgcacggcaaac | |
| | | agacagauccauuauuauuacaauuuauuuagugaucguucccgguuuuauac | |
| Aco1_ncRNA | Aco1 ncRNA containing 25 | guauaaaaccgggaacgaucagaccggggugaauucgcccccuugaucaaacggc | SEQ ID NO: |
| only | bp insertion in EMX1 gene | acuaaccacuguuugccgugcgugcguacaaacggcagaagcuggaggaggaag | 19288 |
| | | ggccugaguccgagcagaagaaaACUCGAGCUCUGAAUGACUCC | |
| | | UAUAaaguucucccaucacaucaaccgguggcgcauugccacgaagcaggcca | |
| | | auggggaggacaucgaugucaacgcacgcacggcaaacagacagauccauuauua | |
| | | uuacaauuuauuuagugaucguucccgguuuuauac | |
| Aco1_ncRNA_ | Aco1 ncRNA containing 25 | guauaaaaccgggaacgaucagaccggggugaauucgcccccuugaucaaacggc | SEQ ID NO: |
| AAVS1 | bp insertion in AAVS1 | acuaaccacuguuugccguggugcguUCUGUCCCCUCCACCCCA | 19289 |
| gRNA_25 | gene fused to AAVS1 | CAGUGGGGCCACUAGGGACAGAAAUGACAGUGGUUG | |
| | sgRNA at 3′ end | GUGCUCUAAAAAUUAAUGACAGAAAAGCCCCAUCCU | |
| | | UAGGCCUCCUCCUUCCUAGUCUCCUGAUAUUGGGUC | |
| | | UAACCCCCAacgcacgcacggcaaacagacagauccauuauuuuacaauu | |
| | | uauuuagugaucguucccgguuuuauacAAUAACAACGGGGCCAC | |
| | | UAGGGACAGGAUguuucagagcuaugcuggaaacagcauagcaaguug | |
| | | aaauaaggcuaguccguuaucaacuugaaaaguggcaccgagucggugcuuuu | |
| | | u | |
| Aco1_ncRNA_ | Aco1 ncRNA containing 25 | guauaaaaccgggaacgaucagaccggggugaauucgcccccuugaucaaacggc | SEQ ID NO: |
| EMX1 | bp insertion in EMXI gene | acuaaccacuguuugccgugcgugcguacaaacggcagaagcuggaggaggaag | 19290 |
| gRNA_25 | fused to EMX1 sgRNA at | ggccugaguccgagcagaagaaaACUCGAGCUCUGAAUGACUCC | |
| | 3′ end | UAUAaaguucucccaucacaucaaccgguggegcauugccacgaagcaggcca | |
| | | auggggaggacaucgaugucaacgcacgcacggcaaacagacagauccauuauua | |
| | | uuacaauuuauuugugaucguucccgguuuuauacAAUAACAACgagu | |
| | | ccgagcagaagaagaaguuucagagcuaugcuggaaacagcauagcaaguugaaa | |
| | | uaaggcuaguccguuaucaacuugaaaaguggcaccgagucggugcuuuuu | |
| Aco1_ncRNA_ | Aco1 ncRNA containing 50 | guauaaaaccgggaacgaucagaccggggugaauucgcccccuugaucaaacggc | SEQ ID NO: |
| EMX1 | bp insertion in EMX1 gene | acuaaccacuguuugccgugcgugcguacaaacggcagaagcuggaggaggaag | 19291 |
| gRNA_50 | fused to EMX1 sgRNA at | ggccugaguccgagcagaagaaaACUCGAGCUCUGAAUGACUCC | |
| | 3′ end | UAUAaaguucucccaucacaucaaccgguggcgcauugccacgaagcaggcca | |
| | | auggggaggacaucgaugucaacgcacgcacggcaaacagacagauccauuauua | |
| | | uuacaauuuauuugugaucguucccgguuuuauacAAUAACAACgagu | |
| | | ccgagcagaagaagaaguuucagagcuaugcuggaaacagcauagcaaguugaaa | |
| | | uaaggcuaguccguuaucaacuugaaaaguggcaccgagucggugcuuuuu | |
| Aco1_ncRNA_ | Aco1 ncRNA containing 75 | guauaaaaccgggaacgaucagaccggggugaauucgcccccuugaucaaacggc | SEQ ID NO: |
| EMX1 | bp insertion in EMX1 gene | acuaaccacuguuugccgugcgugcguacaaacggcagaagcuggaggaggaag | 19292 |
| gRNA_75 | fused to EMX1 sgRNA at | ggccugaguccgagcagaagaaaACUCGAGCUCUGAGCCCCACC | |
| | 3′ end | GCAGCUUGCCAGCACUUUCAGUAUCAUGGGGCCCAU | |
| | | GGUUGAAUGACUCCUAUAaaguucucccaucacaucaaccggugg | |
| | | cgcauugccacgaagcaggccaauggggaggacaucgaugucaacgcacgcacgg | |
| | | caaacagacagauccauuauuauuacaauuuauuuagugaucguucccgguuuu | |
| | | auacAAUAACAACgaguccgagcagaagaagaaguuucagagcuaugcug | |
| | | gaaacagcauagcaaguugaaauaaggcuaguccguuaucaacuugaaaaagug | |
| | | gcaccgagucggugcuuuuu | |
| Aco1_ncRNA_ | Aco1 ncRNA containing | guauaaaaccgggaacgaucagaccggggugaauucgcccccuugaucaaacggc | SEQ ID NO: |
| EMX1 | 100 bp insertion in EMX1 | acuaaccacuguuugccgugcgugcguacaaacggcagaagcuggaggaggaag | 19293 |
| gRNA_100 | gene fused to EMX1 | ggccugaguccgagcagaagaaaACUCGAGCUCUGAGCCCCACU | |
| | sgRNA at 3′ end | GUCGAGAAGUAUGUAUCUCGCUCCCGCAGCUUGCCA | |
| | | GCACUUUCAGUAUCAUGGGGCCCAUGGUUGAAUGAC | |
| | | UCCUAUAaaguucucccaucacaucaaccgguggcgcauugccacgaagca | |
| | | ggccaauggggaggacaucgaugucaacgcacgcacggcaaacagacagauccau | |
| | | uauuauuacaauuuauuuagugaucguucccgguuuuauacAAUAACAA | |
| | | Cgaguccgagcagaagaagaaguuucagagcuaugcuggaaacagcauagcaag | |
| | | uugaaauaaggcuaguccguuaucaacuugaaaaaguggcaccgagucggugcu | |
| | | uuuu | |
| Aco1_ncRNA_ | Aco1 ncRNA containing | guauaaaaccgggaacgaucagaccggggugaauucgcccccuugaucaaacggc | SEQ ID NO: |
| EMX1 | GFP gene insertion in | acuaaccacuguuugccgugcgugcguacaaacggcagaagcuggaggaggaag | 19294 |
| gRNA_GFP | EMX1 gene fused to EMX1 | ggccugaguccgagcagaagaacccauauguccuuccgagugagagacacaaaaa | |
| gene | sgRNA at 3′ end. Entire | auuccaacacacuauugcaaugaaaauaaauuuccuuuauuagccagaagucaga | |
| | GFP cassette is in antisense | ugcucaaggggcuucaugauguccccauaauuuuuggcagagggaaaaagaucu | |
| | orientation and contains | cagugguauuugugagccagggcauuggccacaccagccaccaccuucugauag | |
| | mini EF1a promoter and | gcagccugcaccugaggagugcggccgcuuuacuuguacagcucguccaugccg | |
| | beta globin polyA signal. | agagugaucccggcggcggucacgaacuccagcaggaccaugugaucgcgcuuc | |
| | | ucguuggggucuuugcucagggcggacugggugcucagguagugguugucggg | |
| | | cagcagcacggggccgucgccgauggggguguucugcugguaguggucggcga | |
| | | gcugcacgcugccguccucgauguuguggeggaucuugaaguucaccuugaugc | |
| | | cguucuucugcuugucggccaugauauagacguuguggcuguuguaguuguac | |
| | | uccagcuugugccccaggauguugccguccuccuugaagucgaugcccuucagc | |
| | | ucgaugegguucaccagggugugcccucgaacuucaccuggggggucu | |
| | | uaguugccgucguccuugaagaagaugguggcuccuggaguagccuucgggc | |
| | | auggcggacuugaagaagucgugcugcuucauguggucgggguagggcugaa | |
| | | gcacugcacgccguaggucaggguggucacgagggugggccagggcacgggcag | |
| | | cuugccgguggugcagaugaacuucagggucagcuugccguagguggcaucgcc | |
| | | cucgcccucgccggacacgcugaacuuguggccguuuacguegccguccagcuc | |
| | | gaccaggaugggcaccaccccggugaacagcuccucgcccuugcucaccauggug | |
| | | gcgaccgguggaucccgggcccgcgguaccgucgacugcagaauucgaagcuug | |
| | | agcucgagaucugaguccgguagegcuageggaucugacgguucacuaaacccu | |
| | | guguucuggeggcaaacccguugcgaaaaagaacguucacggcgacuacugcac | |
| | | uuauauacgguucucccccacccucgggaaaaaggeggagccaguacacgacauc | |
| | | acuuucccaguuuaccccgcgccaccuucucuaggcaccgguucaauugccgacc | |
| | | ccuccccccaacuucucggggucugugggcguugugcgcucugcccguucuccc | |
| | | aucacaucaaccgguggcgcauugccacgaagcaggccaauggggaggacaucga | |
| | | ugucaacgcacgcacggcaaacagacagauccauuauuauuacaauuuauuuagu | |
| | | gaucguucccgguuuuauacAAUAACAACgaguccgagcagaagaagaag | |
| | | uuucagagcuaugcuggaaacagcauagcaaguugaaauaaggcuaguccguua | |
| | | ucaacuugaaaaaguggcaccgagucggugcuuuuu | |
| Aco1_NT_ | Aco1 ncRNA containing 25 | guauaaaaccgggaacgaucagaccggggugaauucgcccccuugaucaaacggc | SEQ ID NO: |
| ncRNA_EMX1 | bp insertion (on opposite | acuaaccacuguuugccgugcgugcguugacaucgauguccuccccauuggccu | 19295 |
| gRNA_25 | strand of sgRNA cut) in | gcuucguggcaaugcgccaccgguugaugugaugggagaacuuUAUAGGA | |
| | EMX1 gene fused to EMX1 | GUCAUUCAGAGCUCGAGUuuucuucugcucggacucaggcecuuc | |
| | sgRNA at 3′ end | cuccuccagcuucugccguuuguacgcacgcacggcaaacagacagauccauuau | |
| | | uauuacaauuuauuuagugaucguucccgguuuuauacAAUAACAACga | |
| | | guccgagcagaagaagaaguuucagagcuaugcuggaaacagcauagcaaguug | |
| | | aaauaaggcuaguccguuaucaacuugaaaaaguggcaccgagucggugcuuuu | |
| | | u | |
| R2042_EMX1 | R2042 ncRNA containing | gaguccgagcagaagaagaaguuucagagcuaugcuggaaacagcauagcaagu | SEQ ID NO: |
| gRNA_ncRNA_ | 25 bp insertion in EMX1 | ugaaauaaggcuaguccguuaucaacuugaaaaaguggcaccgagucggugcuu | 19296 |
| 25 | gene fused to EMX1 | uuuAAUAACAACGCGUUAAGGUGGUUAUAUUCUAGU | |
| | sgRNA at 5′ end | AUUUAUGAAGUGUAGUCGCUUCGAUCGUUAAGGCUG | |
| | | AUUUUAACCUCUGCAUAAUAAUAUCGGUAGAUAUUA | |
| | | UUAUGCACGCUCCCUUUAGCAGAGCUAAGAAUCGCU | |
| | | CACUCAGGCACAAGCUUUGAGGACAAACGGCAGAAG | |
| | | CUGGAGGAGGAAGGGCCUGAGUCCGAGCAGAAGAAa | |
| | | ACUCGAGCUCUGAAUGACUCCUAUAAAGUUCUCCCA | |
| | | UCACAUCAACCGGUGGCGCAUUGCCACGAAGCAGGC | |
| | | CAAUGGGGAGGACAUCGAUGUCACCUCAAAGCUUGU | |
| | | GCCUGAGUGAGAGCUAAAGAAAAGAAAAGUAGAAU | |
| | | AAGCCACCUUAACGC | |
| R2042_ncRNA | R2042 ncRNA containing | GCGUUAAGGUGGUUAUAUUCUAGUAUUUAUGAAGU | SEQ ID NO: |
| only_25 | 25 bp insertion in EMX1 | GUAGUCGCUUCGAUCGUUAAGGCUGAUUUUAACCUC | 19297 |
| | gene | UGCAUAAUAAUAUCGGUAGAUAUUAUUAUGCACGCU | |
| | | CCCUUUAGCAGAGCUAAGAAUCGCUCACUCAGGCAC | |
| | | AAGCUUUGAGGACAAACGGCAGAAGCUGGAGGAGGA | |
| | | AGGGCCUGAGUCCGAGCAGAAGAAaACUCGAGCUCU | |
| | | GAAUGACUCCUAUAAAGUUCUCCCAUCACAUCAACC | |
| | | GGUGGCGCAUUGCCACGAAGCAGGCCAAUGGGGAGG | |
| | | ACAUCGAUGUCACCUCAAAGCUUGUGCCUGAGUGAG | |
| | | AGCUAAAGAAAAGAAAAGUAGAAUAAGCCACCUUAA | |
| | | CGC | |
| R2042_ncRNA_ | R2042 ncRNA containing | GCGUUAAGGUGGUUAUAUUCUAGUAUUUAUGAAGU | SEQ ID NO: |
| AAVS1 | 25 bp insertion in EMX1 | GUAGUCGCUUCGAUCGUUAAGGCUGAUUUUAACCUC | 19298 |
| gRNA_25 | gene fused to AAVS1 | UGCAUAAUAAUAUCGGUAGAUAUUAUUAUGCACGCU | |
| | sgRNA at 3′ end | CCCUUUAGCAGAGCUAAGAAUCGCUCACUCAGGCAC | |
| | | AAGCUUUGAGGUCUGUCCCCUCCACCCCACAGUGGG | |
| | | GCCACUAGGGACAGAAAUGACAGUGGUUGGUGCUCU | |
| | | AAAAAUUAAUGACAGAAAAGCCCCAUCCUUAGGCCU | |
| | | CCUCCUUCCUAGUCUCCUGAUAUUGGGUCUAACCCC | |
| | | CACCUCAAAGCUUGUGCCUGAGUGAGAGCUAAAGAA | |
| | | AAGAAAAGUAGAAUAAGCCACCUUAACGCAAUAACA | |
| | | ACGGGGCCACUAGGGACAGGAUguuucagagcuaugcuggaaa | |
| | | cagcauagcaaguugaaauaaggcuaguccguuaucaacuugaaaaaguggcacc | |
| | | gagucggugcuuuuu | |
| R2042_ncRNA_ | R2042 ncRNA containing | GCGUUAAGGUGGUUAUAUUCUAGUAUUUAUGAAGU | SEQ ID NO: |
| EMX1 | 25 bp insertion in EMX1 | GUAGUCGCUUCGAUCGUUAAGGCUGAUUUUAACCUC | 19299 |
| gRNA_25 | gene fused to EMX1 | UGCAUAAUAAUAUCGGUAGAUAUUAUUAUGCACGCU | |
| | sgRNA at 3′ end | CCCUUUAGCAGAGCUAAGAAUCGCUCACUCAGGCAC | |
| | | AAGCUUUGAGGACAAACGGCAGAAGCUGGAGGAGGA | |
| | | AGGGCCUGAGUCCGAGCAGAAGAAaACUCGAGCUCU | |
| | | GAAUGACUCCUAUAAAGUUCUCCCAUCACAUCAACC | |
| | | GGUGGCGCAUUGCCACGAAGCAGGCCAAUGGGGAGG | |
| | | ACAUCGAUGUCACCUCAAAGCUUGUGCCUGAGUGAG | |
| | | AGCUAAAGAAAAGAAAAGUAGAAUAAGCCACCUUAA | |
| | | CGCAAUAACAACgaguccgagcagaagaagaaguuucagagcuaugcu | |
| | | ggaaacagcauagcaaguugaaauaaggcuaguccguuaucaacuugaaaaagu | |
| | | ggcaccgagucggugcuuuuu | |
| R2042_ncRNA_ | R2042 ncRNA containing | GCGUUAAGGUGGUUAUAUUCUAGUAUUUAUGAAGU | SEQ ID NO: |
| EMX1 | 50 bp insertion in EMX1 | GUAGUCGCUUCGAUCGUUAAGGCUGAUUUUAACCUC | 19300 |
| gRNA_50 | gene fused to EMX1 | UGCAUAAUAAUAUCGGUAGAUAUUAUUAUGCACGCU | |
| | sgRNA at 3′ end | CCCUUUAGCAGAGCUAAGAAUCGCUCACUCAGGCAC | |
| | | AAGCUUUGAGGACAAACGGCAGAAGCUGGAGGAGGA | |
| | | AGGGCCUGAGUCCGAGCAGAAGAAaACUCGAGCUCU | |
| | | GAUCAGUAUCAUGGGGCCCAUGGUUGAAUGACUCCU | |
| | | AUAaaguuCUCCCAUCACAUCAACCGGUGGCGCAUUGC | |
| | | CACGAAGCAGGCCAAUGGGGAGGACAUCGAUGUCAC | |
| | | CUCAAAGCUUGUGCCUGAGUGAGAGCUAAAGAAAAG | |
| | | AAAAGUAGAAUAAGCCACCUUAACGCAAUAACAACga | |
| | | guccgagcagaagaagaaguuucagagcuaugcuggaaacagcauagcaaguug | |
| | | u | |
| R2042_ncRNA_ | R2042 ncRNA containing | GCGUUAAGGUGGUUAUAUUCUAGUAUUUAUGAAGU | SEQ ID NO: |
| EMX1 | 75 bp insertion in EMX1 | GUAGUCGCUUCGAUCGUUAAGGCUGAUUUUAACCUC | 19301 |
| gRNA_75 | gene fused to EMX1 | UGCAUAAUAAUAUCGGUAGAUAUUAUUAUGCACGCU | |
| | sgRNA at 3′ end | CCCUUUAGCAGAGCUAAGAAUCGCUCACUCAGGCAC | |
| | | AAGCUUUGAGGACAAACGGCAGAAGCUGGAGGAGGA | |
| | | AGGGCCUGAGUCCGAGCAGAAGAAaACUCGAGCUCU | |
| | | GAGCCCCACCGCAGCUUGCCAGCACUUUCAGUAUCA | |
| | | UGGGGCCCAUGGUUGAAUGACUCCUAUAaaguuCUCCC | |
| | | AUCACAUCAACCGGUGGCGCAUUGCCACGAAGCAGG | |
| | | CCAAUGGGGAGGACAUCGAUGUCACCUCAAAGCUUG | |
| | | UGCCUGAGUGAGAGCUAAAGAAAAGAAAAGUAGAA | |
| | | UAAGCCACCUUAACGCAAUAACAACgaguccgagcagaagaa | |
| | | gaaguuucagagcuaugcuggaaacagcauagcaaguugaaauaaggcuagucc | |
| | | guuaucaacuugaaaaaguggcaccgagucggugcuuuuu | |
| R2042_ncRNA_ | R2042 ncRNA containing | GCGUUAAGGUGGUUAUAUUCUAGUAUUUAUGAAGU | SEQ ID NO: |
| EMX1 | 100 bp insertion in EMX1 | GUAGUCGCUUCGAUCGUUAAGGCUGAUUUUAACCUC | 19302 |
| gRNA_100 | gene fused to EMX1 | UGCAUAAUAAUAUCGGUAGAUAUUAUUAUGCACGCU | |
| | sgRNA at 3′ end | CCCUUUAGCAGAGCUAAGAAUCGCUCACUCAGGCAC | |
| | | AAGCUUUGAGGACAAACGGCAGAAGCUGGAGGAGGA | |
| | | AGGGCCUGAGUCCGAGCAGAAGAAaACUCGAGCUCU | |
| | | GAGCCCCACUGUCGAGAAGUAUGUAUCUCGCUCCCG | |
| | | CAGCUUGCCAGCACUUUCAGUAUCAUGGGGCCCAUG | |
| | | GUUGAAUGACUCCUAUAaaguuCUCCCAUCACAUCAAC | |
| | | CGGUGGCGCAUUGCCACGAAGCAGGCCAAUGGGGAG | |
| | | GACAUCGAUGUCACCUCAAAGCUUGUGCCUGAGUGA | |
| | | GAGCUAAAGAAAAGAAAAGUAGAAUAAGCCACCUUA | |
| | | ACGCAAUAACAACgaguccgagcagaagaagaaguuucagagcuaugc | |
| | | uggaaacagcauagcaaguugaaauaaggcuaguccguuaucaacuugaaaaag | |
| | | uggcaccgagucggugcuuuuu | |
| R2042_ncRNA_ | R2042 ncRNA containing | GCGUUAAGGUGGUUAUAUUCUAGUAUUUAUGAAGU | SEQ ID NO: |
| NT_EMX1 | 25 bp insertion (on opposite | GUAGUCGCUUCGAUCGUUAAGGCUGAUUUUAACCUC | 19303 |
| gRNA_25_ | strand of sgRNA cut) in | UGCAUAAUAAUAUCGGUAGAUAUUAUUAUGCACGCU | |
| dua1 guides | EMX1 gene fused to EMX1 | CCCUUUAGCAGAGCUAAGAAUCGCUCACUCAGGCAC | |
| | sgRNA at 3′ end | AAGCUUUGAGGugacaucgauguccuccccauuggccugcuucguggc | |
| | | aaugcgccaccuauaguuaguguacugcaacuuUAUAGGAGUCAUUC | |
| | | AGAGCUCGAGUuuucuucugcucggacucaggcccuuccuccuccagcu | |
| | | ucugccguuuguCCUCAAAGCUUGUGCCUGAGUGAGAGCU | |
| | | AAAGAAAAGAAAAGUAGAAUAAGCCACCUUAACGCA | |
| | | AUAACAACgaguccgagcagaagaagaaguuucagagcuaugcuggaaac | |
| | | agcauagcaaguugaaauaaggcuaguccguuaucaacuugaaaaaguggcacc | |
| | | gagucggugcuuuuu | |
| R2042_RT | R2042 RT mRNA | AUGAAGGACGACCAGUACUCUCAGUGGAAGAAGUAC | SEQ ID NO: |
| | sequence, coding sequence | UACGAGAGCAGGGGCAUCCUGCCCGAGAUCCAGGAC | 19304 |
| | only | AAGCUCCUGAACUACGCCAAGAUCCACAUCGACAAC | |
| | | AACACCCCGGUGAUCUUCAACUUCGAGCACCUGACC | |
| | | CUGCUGCUGGGCAGGGAGAAGAACUACCUGUCCAGC | |
| | | GUGGUGAACAGCCCCGACAGCCACUACAGAAAGUUC | |
| | | AAGAUCAAGAAGAGAUCCGGAGGCGAGAGGGAGAUC | |
| | | ACCGCUCCCUAUCUCAGCCUCCUGGAGAUGCAGUAC | |
| | | UGGAUCUACAGGAACAUCCUGAUCAACGUGAAGAUC | |
| | | CACUACGCCGCUCACGGCUUCGCUCAGGAUAAGAGC | |
| | | AUUAUCACCAACUCCAGGAACCACCUGGGGCAGAAA | |
| | | CAUCUGCUCAAGAUGGAUCUGAAGGAUUUCUUCCCC | |
| | | AGCAUCAAGCUGAACAGAAUCAUCUACAUCUUCAAG | |
| | | AGCCUGGGCUACCCCAAUAUCAUCGCCUUCUACCUG | |
| | | GCCAGCAUCUGCAGCUACAAGGGCCACCUGCCCCAG | |
| | | GGCAGCCCCACAAGCCCUAUCCUGAGCAACAUCGUG | |
| | | AGCAUCACCCUGGACAACAGACUGGUGAAGUUCGCC | |
| | | AGAAAGAUGAAGCUGAGAUACAGCAGGUACGCCGAC | |
| | | GACCUGACGUUCAGCGGGGACAAGAUCCCCACCAAC | |
| | | UACAUCAAGUACAUCACCGACAUCAUCAAUGAUGAG | |
| | | GGCUUCGAGGUGAACGACACCAAGACCAAGCUCUAC | |
| | | CUGAAGGCCGGGAAGAGAAUCGUGACGGGCAUUUCC | |
| | | GUGAUCGGAAAUGACCCGAAGCUUCCGCGGGAAUAC | |
| | | AAGCGGAAGCUGAAGCAGGAGCUGCACUACAUCUUC | |
| | | ACCUACGGCAUCGGCAGCCACAUGGCCAAGAAGAAG | |
| | | AUCAAGAAGAUCAACUACCUCUACAGGAUCAUCGGC | |
| | | AAGGUGAACUUCUGGCUGAACAUCGAGCCCGACAAC | |
| | | GAGUACGCCAGAAACGCCAAGGCCAAGCUGCUGCUG | |
| | | CUCAUCGACAACccaccaaagaagaaaagaaaggucuga | |
| R6943 RT | R6943 RT mRNA | AUGGAGGAGAGCACCAACUACAAGCUGCUGGUGUGG | SEQ ID NO: |
| | sequence, coding sequence | GGACUGAGCGUGAUCCAACCCGCUACCCCCAACGAG | 19305 |
| | only | GUGCUGAACUACCUCACCAGCACCCUGAACGAUAAC | |
| | | GGGCUGCUGCCCGACGUGGAGAAGAUGAUCCACUAC | |
| | | UUUGAGCUGCUGGACCAGCUGGGCUACAUCCACCAG | |
| | | GUGAGCAAGAGGAACAACCUCUACUCACUGACCCCC | |
| | | AGGGGCAACGAAAGGUUGACCCCUGCCCUGAAGAGA | |
| | | CUCAGGGACAAGAUCAGACUGUUCAUGCUGGACAAC | |
| | | UGCCACAGCAUCAGCAAGCUGGGCGUGCUGGCCAGC | |
| | | ACAGAUACAGAGAACAUGGGGGGCGACAGCCCCUCA | |
| | | CUCCAGCUGAGGCACAACCUCAAGGAGGUGCCUCAU | |
| | | CCCAGCCUUAGCUGGGCUGCUGGAACCCUGCCUAGC | |
| | | UCUCCUAGGCAGGCUUGGGUUCGGAUCUACGAACAG | |
| | | CUGAACAUCGGCAGCAUGAGCAGCGACGAGGCUAGC | |
| | | ACACCCACCACCGCCAGAAAUGCCCCCCUGAGCUUCG | |
| | | UGGGCAGGCUGGGCUUCAGCCUGAACUACUACAGCU | |
| | | UCAACAAGAUCGACGAGCCCCUGUUCAACAACGAUG | |
| | | GCGUGACCGCCAUCGCCAGCUGCAUCGGCAUCAGCC | |
| | | CCGGGCUGAUUACCGCUAUGGUGAAGUCACCAAAGC | |
| | | GGUACUACAGGACCUUCAACCUGAGAAAGAAGUCCG | |
| | | GGGGCUUCAGAUCCAUUCUGGCCCCCAGAAAGUUCA | |
| | | UCAAGACCAUCCAGUACUGGCUGAAGGAUCAUGUGC | |
| | | UGAACAGGCUCAAGAUCCACAGCUCCUGUUACAGCU | |
| | | ACAGGAGCGGCGUGUCCAUCAAGGACAACGCCAUCA | |
| | | ACCACGUGAAGAAGAAGUUCGUGGCCAGCAUCGACA | |
| | | UUUCCGAUUACUUCGGAAGCAUCAACAAGAAGAUGG | |
| | | UGAAGGACUGCUUUUACAAGAACAAUAUUCCCGAUC | |
| | | ACAUCGUGAAUACCAUCAGCGGCAUCGUGACCUACA | |
| | | ACGACGUGCUGCCUCAGGGCGCUCCCACCAGCCCUA | |
| | | UCAUUAGCAACGCCAUCCUGUUCGAGUUCGACGAGG | |
| | | AGAUGACGGCUCAUGCCCUCACUCUCGACUGUAUCU | |
| | | ACACCAGAUACAGCGACGACAUCUCGAUAUCCUCCG | |
| | | ACUAUAAGGAGAAUAUCGCCAUCCUGAUCAACAUCG | |
| | | CCGAGGCCAACCUGUUGAGCGCUGGAUUCACGCUCA | |
| | | ACAGACAGAAGCAAAGGAUUGCUUCUGACAACAGCC | |
| | | GCCAGGUUGUGACCGGCAUCCUGGUGAACGAGAGCA | |
| | | UCAGACCCACCAGAUGCUACAGAAAGAAGAUCAGAA | |
| | | GCGCCUUUGAUCACGCCCUGAAGGAGCAGGACGGCU | |
| | | CCCAGCUGACAAUCAACAAGUUGAGGGGCUACCUCA | |
| | | ACUACCUGAAGUCCUUCGAGACCUACGGCUUCAAGU | |
| | | UCAACGAGAAGAAGUAUAAGGAGACCCUGGAUUUCC | |
| | | UGAUCGCUCUGAAGCAGAGCccaccaaagaagaaaagaaaggucu | |
| | | ga | |
| R6943_ncRNA_ | R6943 ncRNA containing | GCGGAGUGCUGGCCUCAACUGAUACAGAGAAUAUGG | SEQ ID NO: |
| EMX1gRNA_ | GFP gene insertion in | GCGGUGAUUCGCCGUCUUUACAGUUAAGGCACAAUU | 19306 |
| GFP gene | EMX1 gene fused to EMX1 | UAAAAGAGGUUCCGCACCCAAGCCUGUCUUGGGCUG | |
| | sgRNA at 3′ end. Entire | Cacaaacggcagaagcuggaggaggaagggccugaguccgagcagaagaaccca | |
| | GFP cassette is in antisense | uauguccuuccgagugagagacacaaaaaauuccaacacacuauugcaaugaaaa | |
| | orientation and contains | uaaauuuccuuuauuagccagaagucagaugcucaaggggcuucaugauguccc | |
| | mini EFla promoter and | cauaauuuuuggcagagggaaaaagaucucagugguauuugugagccagggcau | |
| | beta globin polyA signal. | uggccacaccagccaccaccuucugauaggcagccugcaccugaggagugeggcc | |
| | | gcuuuacuuguacagcucguccaugccgagagugaucccggggggucacgaa | |
| | | cuccagcaggaccaugugaucgcgcuucucguuggggucuuugcucagggcgga | |
| | | cugggugcucagguagugguugucgggcagcagcacggggccgucgccgaugg | |
| | | ggguguucugcugguaguggucggcgagcugcacgcugccguccucgauguug | |
| | | uggcggaucuugaaguucaccuugaugccguucuucugcuugucggccaugau | |
| | | auagacguuguggcuguuguaguuguacuccagcuugugccccaggauguugc | |
| | | cguccuccuugaagucgaugcccuucagcucgaugcgguucaccagggugucgc | |
| | | ccucgaacuucaccucggcgcgggucuuguaguugccgucguccuugaagaaga | |
| | | uggugcgcuccuggacguagccuucgggcauggggacuugaagaagucgugc | |
| | | ugcuucauguggucgggguagcggcugaagcacugcacgccguaggucagggu | |
| | | ggucacgagggugggccagggcacgggcagcuugccgguggugcagaugaacu | |
| | | ucagggucagcuugccguagguggcaucgcccucgcccucgccggacacgcuga | |
| | | acuuguggccguuuacgucgccguccagcucgaccaggaugggcaccaccccgg | |
| | | ugaacagcuccucgcccuugcucaccaugguggegaccgguggauccegggccc | |
| | | gcgguaccgucgacugcagaauucgaagcuugagcucgagaucugaguccggua | |
| | | gcgcuagggaucugacgguncacuaaacccuguguucuggggcaaacccguu | |
| | | gcgaaaaagaacguucacggegacuacugcacuuauauacgguucucccccaccc | |
| | | ucgggaaaaaggeggagccaguacacgacaucacuuucccaguuuaccccgegcc | |
| | | accuucucuaggcacegguucaauugcegaccccuccccccaacuucucggggac | |
| | | ugugggcgaugugegcucugcccguucucccaucacaucaaccgguggcgcauu | |
| | | gccacgaagcaggccaauggggaggacaucgaugucaGCAGCCCAAGAC | |
| | | AGGCUUGGGUGCGGAUCUACGAGCAAUUAAAUAUUG | |
| | | GUUCGAUGUCCAGUGAUGAGGCCAGCACCCGCAAUA | |
| | | ACAACgaguccgagcagaagaagaaguuucagagcuaugcuggaaacagcau | |
| | | agcaaguugaaauaaggcuaguccguuaucaacuugaaaaaguggcaccgaguc | |
| | | ggugcuuuuu | |
| R6943_ncRNA_ | R6943 ncRNA containing | GCGGAGUGCUGGCCUCAACUGAUACAGAGAAUAUGG | SEQ ID NO: |
| EMX1gRNA_ | 100 bp insertion in EMX1 | GCGGUGAUUCGCCGUCUUUACAGUUAAGGCACAAUU | 19307 |
| 100 bp | gene fused to EMX1 | UAAAAGAGGUUCCGCACCCAAGCCUGUCUUGGGCUG | |
| | sgRNA at 3′ end | CacaaacggcagaagcuggaggaggaagggccugaguccgagcagaagaaaAC | |
| | | UCGAGCUCUGAGCCCCACUGUCGAGAAGUAUGUAUC | |
| | | UCGCUCCCGCAGCUUGCCAGCACUUUCAGUAUCAUG | |
| | | GGGCCCAUGGUUGAAUGACUCCUAU Aaaguucucccaucac | |
| | | aucaaccgguggcgcauugccacgaagcaggccaauggggaggacaucgauguc | |
| | | aGCAGCCCAAGACAGGCUUGGGUGCGGAUCUACGAG | |
| | | CAAUUAAAUAUUGGUUCGAUGUCCAGUGAUGAGGCC | |
| | | AGCACCCGCAAUAACAACgaguccgagcagaagaagaaguuucaga | |
| | | gcuaugcuggaaacagcauagcaaguugaaauaaggcuaguccguuaucaacuu | |
| | | gaaaaaguggcuccgagucggugcuuuuu | |
| R6943_ncRNA_ | R6943 ncRNA containing | GCGGAGUGCUGGCCUCAACUGAUACAGAGAAUAUGG | SEQ ID NO: |
| EMX1gRNA_ | 75 bp insertion in EMX1 | GCGGUGAUUCGCCGUCUUUACAGUUAAGGCACAAUU | 19308 |
| 75 bp | gene fused to EMX1 | UAAAAGAGGUUCCGCACCCAAGCCUGUCUUGGGCUG | |
| | sgRNA at 3′ end | CacaaacggcagaagcuggaggaggaagggccugaguccgagcagaagaaaAC | |
| | | UCGAGCUCUGAGCCCCACCGCAGCUUGCCAGCACUU | |
| | | UCAGUAUCAUGGGGCCCAUGGUUGAAUGACUCCUAU | |
| | | Aaaguucucccaucacaucaaccgguggcgcauugccacgaagcaggccaaugg | |
| | | ggaggacaucgaugucaGCAGCCCAAGACAGGCUUGGGUGCG | |
| | | GAUCUACGAGCAAUUAAAUAUUGGUUCGAUGUCCAG | |
| | | UGAUGAGGCCAGCACCCGCAAUAACAACgaguccgagcag | |
| | | aagaagaaguuucagagcuaugcuggaaacagcauagcaaguugaaauaaggcu | |
| | | aguccguuaucaacuugaaaaaguggcaccgagucggugcuuuuu | |
| R6943_ncRNA_ | R6943 ncRNA containing | GCGGAGUGCUGGCCUCAACUGAUACAGAGAAUAUGG | SEQ ID NO: |
| EMX1gRNA_ | 50 bp insertion in EMX1 | GCGGUGAUUCGCCGUCUUUACAGULAAGGCACAAUU | 19309 |
| 50 bp | gene fused to EMX1 | UAAAAGAGGUUCCGCACCCAAGCCUGUCUUGGGCUG | |
| | sgRNA at 3′ end | CacaaacggcagaagcuggaggaggaagggccugaguccgagcagaagaaaAC | |
| | | UCGAGCUCUGAUCAGUAUCAUGGGGCCCAUGGUUGA | |
| | | AUGACUCCUAUAaaguucucccaucacaucaaccgguggcgcauugcca | |
| | | cgaagcaggccaauggggaggacaucgaugucaGCAGCCCAAGACAG | |
| | | GCUUGGGUGCGGAUCUACGAGCAAUUAAAUAUUGGU | |
| | | UCGAUGUCCAGUGAUGAGGCCAGCACCCGCAAUAAC | |
| | | AACgaguccgagcagaagaagaaguuucagagcuaugcuggaaacagcauagc | |
| | | aaguugaaauaaggcuaguccguuaucaacuugaaaaaguggcaccgagucggu | |
| | | gcuuuuu | |
| R6943_ncRNA_ | R6943 ncRNA containing | GCGGAGUGCUGGCCUCAACUGAUACAGAGAAUAUGG | SEQ ID NO: |
| EMX1gRNA_ | 25 bp insertion in EMX1 | GCGGUGAUUCGCCGUCUUUACAGUUAAGGCACAAUU | 19310 |
| 25 bp | gene fused to EMX1 | UAAAAGAGGUUCCGCACCCAAGCCUGUCUUGGGCUG | |
| | sgRNA at 3′ end | CacaaacggcagaagcuggaggaggaagggccugaguccgagcagaagaaaAC | |
| | | UCGAGCUCUGAAUGACUCCUAUAaaguucucccaucacaucaa | |
| | | ccgguggcgcauugccacgaagcaggccaauggggaggacaucgaugucaGCA | |
| | | GCCCAAGACAGGCUUGGGUGCGGAUCUACGAGCAAU | |
| | | UAAAUAUUGGUUCGAUGUCCAGUGAUGAGGCCAGCA | |
| | | CCCGCAAUAACAACgaguccgagcagaagaagaaguuucagagcuau | |
| | | gcuggaaacagcauagcaaguugaaauaaggcuaguccguuaucaacuugaaaaa | |
| | | guggcaccgagucggugcuuuuu | |
| R6943_ncRNA_ | R6943 ncRNA containing | GCGGAGUGCUGGCCUCAACUGAUACAGAGAAUAUGG | SEQ ID NO: |
| AAVS1 | 25 bp insertion in AAVS1 | GCGGUGAUUCGCCGUCUUUACAGUUAAGGCACAAUU | 19311 |
| gRNA_25 bp | gene fused to AAVS1 | UAAAAGAGGUUCCGCACCCAAGCCUGUCUUGGGCUG | |
| | sgRNA at 3′ end | CUCUGUCCCCUCCACCCCACAGUGGGGCCACUAGGG | |
| | | ACAGAAAUGACAGUGGUUGGUGCUCUAAAAAUUAAU | |
| | | GACAGAAAAGCCCCAUCCUUAGGCCUCCUCCUUCCU | |
| | | AGUCUCCUGAUAUUGGGUCUAACCCCCAGCAGCCCA | |
| | | AGACAGGCUUGGGUGCGGAUCUACGAGCAAUUAAAU | |
| | | AUUGGUUCGAUGUCCAGUGAUGAGGCCAGCACCCGC | |
| | | AAUAACAACGGGGCCACUAGGGACAGGAUguuucagagc | |
| | | uaugcuggaaacagcauagcaaguugaaauaaggcuaguccguuaucaacuuga | |
| | | aaaaguggcaccgagucggugcuuuuu | |
| R6943_ncRNA | R6943 ncRNA containing | GCGGAGUGCUGGCCUCAACUGAUACAGAGAAUAUGG | SEQ ID NO: |
| only_25 bp | 25 bp insertion in EMX1 | GCGGUGAUUCGCCGUCUUUACAGUUAAGGCACAAUU | 19312 |
| | gene | UAAAAGAGGUUCCGCACCCAAGCCUGUCUUGGGCUG | |
| | | CacaaacggcagaagcuggaggaggaagggccugaguccgagcagaagaaaAC | |
| | | UCGAGCUCUGAAUGACUCCUAUAaaguucucccaucacaucaa | |
| | | ccgguggcgcauugccacgaagcaggccaauggggaggacaucgaugucaGCA | |
| | | GCCCAAGACAGGCUUGGGUGCGGAUCUACGAGCAAU | |
| | | UAAAUAUUGGUUCGAUGUCCAGUGAUGAGGCCAGCA | |
| | | CCCGC | |
| AAVS1 | Contains chemical | GGGGCCACUAGGGACAGGAUguuucagagcuaugcuggaaacag | SEQ ID NO: |
| sgRNA | modifications from | cauagcaaguugaaauaaggcuaguccguuaucaacuugaaaaaguggcaccga | 19313 |
| | Synthego: 2′-F, 2′-O- | gucggugcuuuuu | |
| | methyl, phosphorothioates | | |
| R1262_RT-1 | R1262 retron RT mRNA | AGGAUAAUGGGAAAUAAGAGAGAAAAGAAGAGUAA | SEQ ID NO: |
| (R1262_RT) | | GAAGAAAUAUAAGAGCCACCAUGAUCAGCUUCAGCG | 19314 |
| | | AGAUCAAGAGCAGAAACGAUUUUGCAGACGCUCUGC | |
| | | AGAUUCCUCGGAGCGUGCUGACGCACGUUCUGUACA | |
| | | UUGCCAAGCCCGAGAGCUUCUACGAGAGCUUCACCA | |
| | | UCCCCAAGAAGAAUGGGGAGGACAGAAUCAUCAUGG | |
| | | CCCCCAAGGGCACCCUGAAGUCCAUCCAGACCAAGC | |
| | | UGAGCAAGCAGCUGGUGGAGUACAGAGCCUCCAUCA | |
| | | GCCAGAAAGGCCAGGAGAAGUCCAACAUCUCCCAUG | |
| | | GCUUCGAGAGGGAGAAGUCCAUCAUCACCAAUGCUC | |
| | | AGAUACACCGCAACAAGCGGUAUGUCAUCAACUACG | |
| | | ACCUGAAGGACUUCUUCGACUCCUUCCACUUCGGCA | |
| | | GAGUGGUGGGAUUCUUCGAGAAGAACAAGCACUUCC | |
| | | UGCUGCCCUACGAGGUGGCCGUGAUCAUCGCCCAGC | |
| | | UGACCUGCUAUAAUGGCAGGCUGCCCCAGGGCGCCC | |
| | | CCACAAGCCCUGUGAUCACCAACCUGAUCUGCGAGA | |
| | | UCCUGGACUACAGGGUGCUCAAGAUCGCCAAGAGAU | |
| | | ACAAGCUGGACUACACCAGGUACGCUGACGACCUGA | |
| | | CCUUCAGCACCAACUACUCCAGAUUCCUGGAGGUGU | |
| | | UCGACAGCUUCGCCAAGGAGCUGCUCCAGGAGAUCA | |
| | | GCAACUCUGGUUUUACCAUAAACCAGAGCAAGACCA | |
| | | GGCUGCUGUACAGAGACAGCCGCCAGGAGGUCACAG | |
| | | GCCUCGUGGUGAAUAAGAAGAUCGGCGUGAACAGAG | |
| | | AGUACGUCAAGAGCACUAGGGCGAUGGCCCAGGCUC | |
| | | UGUACAGCACCGGCGAGUUCACCAUCAACGGCAUCC | |
| | | CCGGCACCAUCAAACAGCUGGAGGGCAGGUUCGGCU | |
| | | UCAUCGACCAGCUGGACCACUACAACAACGUGAUCG | |
| | | ACGAUGCCAAGCACGACGCCUACUCCCUGAACGGCA | |
| | | GGGAGAAGCAGUUCCAGGAGUUCCUGUUCUUCAAGA | |
| | | CAUUCUUCUUCAACGAGUACCCCCUGGUGAUCACCG | SEQ ID NO: |
| | | AGGGCAAGACCGACAUCAGAUACCUGAAGGCCGCCC | |
| | | UCAAGAGCCUGCACCAGAAGUACCCCGAGCUGAUCU | |
| | | GCAAGGAGGACGACGGAACCUUUCGGUUCAAGAUCA | |
| | | GCUUCUUCAGAAGAUCCAAGAGAUGGAAGUACUUCU | |
| | | UCGGCAUCAGCAAGGAUGGCGCUGAUGCCAUGAAGC | |
| | | UGCUGUACCGGUUCUUUACCGGACAGAAGGGCGUGA | |
| | | AGAACUACUACAGGCUGUUCGCCGAGAAGUACAAGG | |
| | | CCGUGCAGAGAAACCCCGUGAUCAUGCUGUUUGACA | |
| | | ACGAGAUGGAGAGCAAGAGACCCCUCAACAAGUUCA | |
| | | UCUCCGAGGAGGUGAAGAUUCCCUCCUCGGAGCAGC | |
| | | AGCUGUUCAAGGAGCAGCUGUACUACCACCUGAUCC | |
| | | CCGGCAGCAAGACCUACCUGAUGACCCACCCCUUGCC | |
| | | UCCUGGCAAGACAGAGGCCGAGAUCGAGGACCUGUU | |
| | | CCCUACCGAGGUGUUGGGCGUGAAGCUCGACGGCAA | |
| | | GAGCUUCAGCACCAAGGACAAGUUCGACACCAGCAA | |
| | | GUUCUACGGCAAGGACAUCUUCAGCAGCUACGUGUA | |
| | | CGAGCACUGGAAGUCCAUCGACUUCAGCGGCUUCAU | |
| | | CCCCCUGCUGGACAAGAUCAACAUGCUCGUGCAGAA | |
| | | CGAGAAGAAGCCCGGGCUGAACACACCACCAAAGAA | |
| | | GAAAAGAAAGGUCUGAGCUGGAGCCUCGGUGGCCAU | |
| | | GCUUCUUGCCCCUUGGGCCUCCCCCCAGCCCCUCCUC | |
| | | CCCUUCCUGCACCCGUACCCCCGUGGUCUUUGAAUA | |
| | | AAGUCUGAGUGGGCGGCGAU | |
| R1262_nc-5 | R1262 ncRNA containing | GGGAUAAUGAGCGUGUGACAAGAUUUUGGGCUUGU | 19315 |
| (R1262_ncRNA- | 505 bp insertion in EMX1 | GUUUCGCAAGCUUUGAUUAAAAUGGUAGAUGGAUU | |
| EMX1_505) | site | UGCUAUCUAUCGUCAUUUCCAGUACUGUUAUGUACA | |
| | | AACGGCAGAAGCUGGAGGAGGAAGGGCCUGAGUCCG | |
| | | AGCAGAAGAAACUUCAGCUAAGGAAGCUACCAAUAU | |
| | | UUAGUUUCUGAGUCUCACGACAGACCUCGCGCGUAG | |
| | | AUUGCCAUGCGUAGAGCUAACGAGCCAGCGGAAAGC | |
| | | GUGAGGCGCUUUUAAGCAUGGCGAGUAAGUGAUCCA | |
| | | ACGCUUCGGAUAUGACUAUAUACUUAGGUUCGAUCU | |
| | | CGUCCCGAGAAUUCUAAGCCUCAACAUCUAUGAGUU | |
| | | AUGAGGUUAGCCGAAAAAGCACGUGGUGGCGCCCAC | |
| | | CGACUGUUCCCAGACUGUAGCUCUUUGUUCUGUCAA | |
| | | GGCCCGACCUUCAUCGCGGCCGAUUCCUUCUGCGGA | |
| | | CCAUACCGUCCUGAUACUUUGGUCAUGUUUCCGUUG | |
| | | UAGGAGUGAACCCACUUGCCUUUGCGUCUUAAUACC | |
| | | AAUGAAAAACCUAUGCACUUUGUACAGGGUACCAUC | |
| | | GGGAUUCUGAACCCUCAGAUAGUGGGGAUCCCGGGU | |
| | | AUAGACCUUUAUCUGCGGUCCAACUUAGGCAUAAAC | |
| | | CUCCAUGCUACAAAGUUCUCCCAUCACAUCAACCGG | |
| | | UGGCGCAUUGCCACGAAGCAGGCCAAUGGGGAGGAC | |
| | | AUCGAUGUCAGCAUAAUGAGUUGUCACACGCUCGAU | |
| R1262_nc-3 | R1262 ncRNA containing | GGGAUAAUGAGCGUGUGACAAGAUUUUGGGCUUGU | SEQ ID NO: |
| (R1262_ncRNA- | 305 bp insertion in EMX1 | GUUUCGCAAGCUUUGAUUAAAAUGGUAGAUGGAUU | 19316 |
| EMX1_305) | site | UGCUAUCUAUCGUCAUUUCCAGUACUGUUAUGUACA | |
| | | AACGGCAGAAGCUGGAGGAGGAAGGGCCUGAGUCCG | |
| | | AGCAGAAGAACGACUACCAAAUCCGCAUGUUACGGG | |
| | | ACUUCUUAUUAAUUCUUUUUUCGUGAGGAGCAGCGG | |
| | | AUCUUAAUGGAUGGCCGCAGGUGGUAUGGAAGCUAA | |
| | | UAGCGCGGGUGAGAGGGUAAUCAGCCGUGUCCACCA | |
| | | ACACAACGCUAUCGGGCGAUUCUAUAAGAUUCCGCA | |
| | | UUGCGUCUACUUAUAAGAUGUCUCAACGGUAUCCGC | |
| | | AACUUGCGAUGUGCCUGCUAUCCUUAAAUGCAUAUC | |
| | | UCGCCCAGUAGCUUCCCAAUAUGAGAGCAUCAAUUG | |
| | | UAGAUCGGGCCGGGAUAGUCAUGUCGUAAAGUUCUC | |
| | | CCAUCACAUCAACCGGUGGCGCAUUGCCACGAAGCA | |
| | | GGCCAAUGGGGAGGACAUCGAUGUCAGCAUAAUGAG | |
| | | UUGUCACACGCUCGAU | |
| R1262_nc-1 | R1262 ncRNA containing | GGGAUAAUGAGCGUGUGACAAGAUUUUGGGCUUGU | SEQ ID NO: |
| (R1262_ncRNA- | 25 bp insertion in EMX1 | GUUUCGCAAGCUUUGAUUAAAAUGGUAGAUGGAUU | 19317 |
| EMX1_25) | site | UGCUAUCUAUCGUCAUUUCCAGUACUGUUAUGUACA | |
| | | AACGGCAGAAGCUGGAGGAGGAAGGGCCUGAGUCCG | |
| | | AGCAGAAGAAAACUCGAGCUCUGAAUGACUCCUAUA | |
| | | AAGUUCUCCCAUCACAUCAACCGGUGGCGCAUUGCC | |
| | | ACGAAGCAGGCCAAUGGGGAGGACAUCGAUGUCAGC | |
| | | AUAAUGAGUUGUCACACGCUCGAU | |
| R1262_nc-4 | R1262 ncRNA containing | GGGAUAAUGAGCGUGUGACAAGAUUUUGGGCUUGU | SEQ ID NO: |
| (R1262_ncRNA- | 405 bp insertion in EMX1 | GUUUCGCAAGCUUUGAUUAAAAUGGUAGAUGGAUU | 19318 |
| EMX1_405) | site | UGCUAUCUAUCGUCAUUUCCAGUACUGUUAUGUACA | |
| | | AACGGCAGAAGCUGGAGGAGGAAGGGCCUGAGUCCG | |
| | | AGCAGAAGAACUGCUAAAUCCGCGUGAUAGGGGAUU | |
| | | UGAAGUUUAAUCUUCUAUCGCAAGGAACUGCCGAUC | |
| | | UUAAUGGAUGGCCGGAGGUGGUAUGGAAGCUAUAA | |
| | | GCGCGGGUGAGAGGGUAAUUAGGCGUGUUCACCUAC | |
| | | GCUACGCUAACGGGCGAUUCUAUAAGAUUGCACAUU | |
| | | GCGUCAACUCAUAAGAUGUCUCAACGGCAUGCGCAA | |
| | | CUUGUGAAGUGUCUACUAUCCUUAAACGCAUAUCUC | |
| | | GCACAGUAACUCCCGAAUAUGUCGGCAUCUGAUGUU | |
| | | GCCCGGGCCGAGUUAGUGUUGAGCUCACGGAACUUA | |
| | | UUGUAUGAGUAGUGAUUUGUAAGAGUUGUCAGUUA | |
| | | GCUCGUUCAGGUAAUAGUUGCCCACACAACGUCAAA | |
| | | AUAAGAGAACGGUCGUAACAUAAAGUUCUCCCAUCA | |
| | | CAUCAACCGGUGGCGCAUUGCCACGAAGCAGGCCAA | |
| | | UGGGGAGGACAUCGAUGUCAGCAUAAUGAGUUGUCA | |
| | | CACGCUCGAU | |
| R1262_nc-2 | R1262 ncRNA containing | GGGAUAAUGAGCGUGUGACAAGAUUUUGGGCUUGU | SEQ ID NO: |
| (R1262_ncRNA- | 205 bp insertion in EMX1 | GUUUCGCAAGCUUUGAUUAAAAUGGUAGAUGGAUU | 19319 |
| EMX1_205) | site | UGCUAUCUAUCGUCAUUUCCAGUACUGUUAUGUACA | |
| | | AACGGCAGAAGCUGGAGGAGGAAGGGCCUGAGUCCG | |
| | | AGCAGAAGAAAAAGCGGUCUUACGGUCAGUCGUAUU | |
| | | CCUUCUCGAGUUCCGUCCAGUUGAGCGUGUCACUCC | |
| | | CAGUGUACCUGCAAGCCGAGAUGGCUGUGCUUGGAG | |
| | | UCAAUCGCAUGUAGGAUGGUCUCCAGACACCGGGGC | |
| | | ACCAGUUUUCACGCCUAAAGCAUAAACGACGAGCAG | |
| | | UCAUGAAAGUCUUAGUACUGGACGUGCCGUUUCACA | |
| | | AAGUUCUCCCAUCACAUCAACCGGUGGCGCAUUGCC | |
| | | ACGAAGCAGGCCAAUGGGGAGGACAUCGAUGUCAGC | |
| | | AUAAUGAGUUGUCACACGCUCGAU | |
| R1262_nc-19 | R1262 ncRNA containing | GGGAUAAUGAGCGUGUGACAAGAUUUUGGGCUUGU | SEQ ID NO: |
| (R1262_ncRNA- | 205 bp insertion in EMX1 | GUUUCGCAAGCUUUGAUUAAAAUGGUAGAUGGAUU | 19320 |
| sgEMX1_205) | site combined with EMX1 | UGCUAUCUAUCGUCAUUUCCAGUACUGUUAUGUACA | |
| | sgRNA at 3′ end | AACGGCAGAAGCUGGAGGAGGAAGGGCCUGAGUCCG | |
| | | AGCAGAAGAAAAAGCGGUCUUACGGUCAGUCGUAUU | |
| | | CCUUCUCGAGUUCCGUCCAGUUGAGCGUGUCACUCC | |
| | | CAGUGUACCUGCAAGCCGAGAUGGCUGUGCUUGGAG | |
| | | UCAAUCGCAUGUAGGAUGGUCUCCAGACACCGGGGC | |
| | | ACCAGUUUUCACGCCUAAAGCAUAAACGACGAGCAG | |
| | | UCAUGAAAGUCUUAGUACUGGACGUGCCGUUUCACA | |
| | | AAGUUCUCCCAUCACAUCAACCGGUGGCGCAUUGCC | |
| | | ACGAAGCAGGCCAAUGGGGAGGACAUCGAUGUCAGC | |
| | | AUAAUGAGUUGUCACACGCUCAAUAACAACGAGUCC | |
| | | GAGCAGAAGAAGAAGUUUCAGAGCUAUGCUGGAAAC | |
| | | AGCAUAGCAAGUUGAAAUAAGGCUAGUCCGUUAUCA | |
| | | ACUUGAAAAAGUGGCACCGAGUCGGUGCUUUUUGAU | |
| R1262_nc-20 | R1262 ncRNA containing | GGGAUAAUGAGUCCGAGCAGAAGAAGAAGUUUCAGA | SEQ ID NO: |
| (R1262_ | 205 bp insertion in EMX1 | GCUAUGCUGGAAACAGCAUAGCAAGUUGAAAUAAGG | 19321 |
| sgEMX1- | site combined with EMX1 | CUAGUCCGUUAUCAACUUGAAAAAGUGGCACCGAGU | |
| ncRNA 205) | sgRNA at 5′ end | CGGUGCUUUUUAAUAACAACGAGCGUGUGACAAGAU | |
| | | UUUGGGCUUGUGUUUCGCAAGCUUUGAUUAAAAUGG | |
| | | UAGAUGGAUUUGCUAUCUAUCGUCAUUUCCAGUACU | |
| | | GUUAUGUACAAACGGCAGAAGCUGGAGGAGGAAGGG | |
| | | CCUGAGUCCGAGCAGAAGAAAAAGCGGUCUUACGGU | |
| | | CAGUCGUAUUCCUUCUCGAGUUCCGUCCAGUUGAGC | |
| | | GUGUCACUCCCAGUGUACCUGCAAGCCGAGAUGGCU | |
| | | GUGCUUGGAGUCAAUCGCAUGUAGGAUGGUCUCCAG | |
| | | ACACCGGGGCACCAGUUUUCACGCCUAAAGCAUAAA | |
| | | CGACGAGCAGUCAUGAAAGUCUUAGUACUGGACGUG | |
| | | CCGUUUCACAAAGUUCUCCCAUCACAUCAACCGGUG | |
| | | GCGCAUUGCCACGAAGCAGGCCAAUGGGGAGGACAU | |
| | | CGAUGUCAGCAUAAUGAGUUGUCACACGCUCGAU | |
| R1262_nc-13 | R1262 ncRNA containing | GGGAUAAUGAGCGUGUGACAAGAUUUUGGGCUUGU | SEQ ID NO: |
| (R1262_ncRNA- | 25 bp insertion in AAVS1 | GUUUCGCAAGCUUUGAUUAAAAUGGUAGAUGGAUU | 19322 |
| AAVS1_25) | site | UGCUAUCUAUCGUCAUUUCCAGUACUGUUAUGUUCU | |
| | | GUCCCCUCCACCCCACAGUGGGGCCACUAGGGACAG | |
| | | AAAGCGGUCUUACGGUCAGUCGUAUUCCUUCUCGAG | |
| | | UUCCGUCCAGUUGAGCGUGUCACUCCCAGUGUACCU | |
| | | GCAAGCCGAGAUGGCUGUGCUUGGAGUCAAUCGCAU | |
| | | GUAGGAUGGUCUCCAGACACCGGGGCACCAGUUUUC | |
| | | ACGCCUAAAGCAUAAACGACGAGCAGUCAUGAAAGU | |
| | | CUUAGUACUGGACGUGCCGUUUCACAAUUAAUGACA | |
| | | GAAAAGCCCCAUCCUUAGGCCUCCUCCUUCCUAGUC | |
| | | UCCUGAUAUUGGGUCUAACCCCCAGCAUAAUGAGUU | |
| | | GUCACACGCUCGAU | |
| R1262_nc-14 | R1262 ncRNA containing | GGGAUAAUGAGCGUGUGACAAGAUUUUGGGCUUGU | SEQ ID NO: |
| (R1262_ncRNA- | 205 bp insertion in AAVS1 | GUUUCGCAAGCUUUGAUUAAAAUGGUAGAUGGAUU | 19323 |
| AAVS1_205) | site | UGCUAUCUAUCGUCAUUUCCAGUACUGUUAUGUUCU | |
| | | GUCCCCUCCACCCCACAGUGGGGCCACUAGGGACAG | |
| | | AAAGCGGUCUUACGGUCAGUCGUAUUCCUUCUCGAG | |
| | | UUCCGUCCAGUUGAGCGUGUCACUCCCAGUGUACCU | |
| | | GCAAGCCGAGAUGGCUGUGCUUGGAGUCAAUCGCAU | |
| | | GUAGGAUGGUCUCCAGACACCGGGGCACCAGUUUUC | |
| | | ACGCCUAAAGCAUAAACGACGAGCAGUCAUGAAAGU | |
| | | CUUAGUACUGGACGUGCCGUUUCACAAUUAAUGACA | |
| | | GAAAAGCCCCAUCCUUAGGCCUCCUCCUUCCUAGUC | |
| | | UCCUGAUAUUGGGUCUAACCCCCAGCAUAAUGAGUU | |
| | | GUCACACGCUCGAU | |
| R1262_nc-15 | R1262 ncRNA containing | GGGAUAAUGAGCGUGUGACAAGAUUUUGGGCUUGU | SEQ ID NO: |
| (R1262_ncRNA- | 505 bp insertion in AAVS1 | GUUUCGCAAGCUUUGAUUAAAAUGGUAGAUGGAUU | 19324 |
| AAVS1_505) | site | UGCUAUCUAUCGUCAUUUCCAGUACUGUUAUGUUCU | |
| | | GUCCCCUCCACCCCACAGUGGGGCCACUAGGGACAG | |
| | | ACUUCAGCUAAGGAAGCUACCAAUAUUUAGUUUCUG | |
| | | AGUCUCACGACAGACCUCGCGCGUAGAUUGCCAUGC | |
| | | GUAGAGCUAACGAGCCAGCGGAAAGCGUGAGGCGCU | |
| | | UUUAAGCAUGGCGAGUAAGUGAUCCAACGCUUCGGA | |
| | | UAUGACUAUAUACUUAGGUUCGAUCUCGUCCCGAGA | |
| | | AUUCUAAGCCUCAACAUCUAUGAGUUAUGAGGUUAG | |
| | | CCGAAAAAGCACGUGGUGGCGCCCACCGACUGUUCC | |
| | | CAGACUGUAGCUCUUUGUUCUGUCAAGGCCCGACCU | |
| | | UCAUCGCGGCCGAUUCCUUCUGCGGACCAUACCGUC | |
| | | CUGAUACUUUGGUCAUGUUUCCGUUGUAGGAGUGAA | |
| | | CCCACCUGCCUUUGCGUCUUAAUACCAAUGAAAAAC | |
| | | CUAUGCACUUUGUACAGGGUACCAUCGGGAUUCUGA | |
| | | ACCCUCAGAUAGUGGGGAUCCCGGGUAUAGACCUUU | |
| | | AUCUGCGGUCCAACUUAGGCAUAAACCUCCAUGCUA | |
| | | CAAUUAAUGACAGAAAAGCCCCAUCCUUAGGCCUCC | |
| | | UCCUUCCUAGUCUCCUGAUAUUGGGUCUAACCCCCA | |
| | | GCAUAAUGAGUUGUCACACGCUCGAU | |
| R1262_nc-18 | R1262 ncRNA containing | GGGAUAAUGAGCGUGUGACAAGAUUUUGGGCUUGU | SEQ ID NO: |
| (R1262_ncRNA- | 505 bp insertion in AAVS1 | GUUUCGCAAGCUUUGAUUAAAAUGGUAGAUGGAUU | 19325 |
| AAVS1- | site and MS2 stem loop at | UGCUAUCUAUCGUCAUUUCCAGUACUGUUAUGUUCU | |
| MS2_505) | 3′ end | GUCCCCUCCACCCCACAGUGGGGCCACUAGGGACAG | |
| | | ACUUCAGCUAAGGAAGCUACCAAUAUUUAGUUUCUG | |
| | | AGUCUCACGACAGACCUCGCGCGUAGAUUGCCAUGC | |
| | | GUAGAGCUAACGAGCCAGCGGAAAGCGUGAGGCGCU | |
| | | UUUAAGCAUGGCGAGUAAGUGAUCCAACGCUUCGGA | |
| | | UAUGACUAUAUACUUAGGUUCGAUCUCGUCCCGAGA | |
| | | AUUCUAAGCCUCAACAUCUAUGAGUUAUGAGGUUAG | |
| | | CCGAAAAAGCACGUGGUGGCGCCCACCGACUGUUCC | |
| | | CAGACUGUAGCUCUUUGUUCUGUCAAGGCCCGACCU | |
| | | UCAUCGCGGCCGAUUCCUUCUGCGGACCAUACCGUC | |
| | | CUGAUACUUUGGUCAUGUUUCCGUUGUAGGAGUGAA | |
| | | CCCACUUGCCUUUGCGUCUUAAUACCAAUGAAAAAC | |
| | | CUAUGCACUUUGUACAGGGUACCAUCGGGAUUCUGA | |
| | | ACCCUCAGAUAGUGGGGAUCCCGGGUAUAGACCUUU | |
| | | AUCUGCGGUCCAACUUAGGCAUAAACCUCCAUGCUA | |
| | | CAAUUAAUGACAGAAAAGCCCCAUCCUUAGGCCUCC | |
| | | UCCUUCCUAGUCUCCUGAUAUUGGGUCUAACCCCCA | |
| | | GCAUAAUGAGUUGUCACACGCUCACAUGAGGAUCAC | |
| | | CCAUGUGAU | |
| R1262_nc-17 | R1262 ncRNA containing | GGGAUAAUGAGCGUGUGACAAGAUUUUGGGCUUGU | SEQ ID NO: |
| (R1262_ncRNA- | 205 bp insertion in AAVS1 | GUUUCGCAAGCUUUGAUUAAAAUGGUAGAUGGAUU | 19326 |
| AAVS1- | site and MS2 stem loop at | UGCUAUCUAUCGUCAUUUCCAGUACUGUUAUGUUCU | |
| MS2_205) | 3′ end | GUCCCCUCCACCCCACAGUGGGGCCACUAGGGACAG | |
| | | AAAGCGGUCUUACGGUCAGUCGUAUUCCUUCUCGAG | |
| | | UUCCGUCCAGUUGAGCGUGUCACUCCCAGUGUACCU | |
| | | GCAAGCCGAGAUGGCUGUGCUUGGAGUCAAUCGCAU | |
| | | GUAGGAUGGUCUCCAGACACCGGGGCACCAGUUUUC | |
| | | ACGCCUAAAGCAUAAACGACGAGCAGUCAUGAAAGU | |
| | | CUUAGUACUGGACGUGCCGUUUCACAAUUAAUGACA | |
| | | GAAAAGCCCCAUCCUUAGGCCUCCUCCUUCCUAGUC | |
| | | UCCUGAUAUUGGGUCUAACCCCCAGCAUAAUGAGUU | |
| | | GUCACACGCUCACAUGAGGAUCACCCAUGUGAU | |
| R1262_nc-16 | R1262 ncRNA containing | GGGAUAAUGAGCGUGUGACAAGAUUUUGGGCUUGU | SEQ ID NO: |
| (R1262_ncRNA- | 25 bp insertion in AAVS1 | GUUUCGCAAGCUUUGAUUAAAAUGGUAGAUGGAUU | 19327 |
| AAVS1- | site and MS2 stem loop at | UGCUAUCUAUCGUCAUUUCCAGUACUGUUAUGUUCU | |
| MS2_25) | 3′ end | GUCCCCUCCACCCCACAGUGGGGCCACUAGGGACAG | |
| | | AACUCGAGCUCUGAAUGACUCCUAUAAUUAAUGACA | |
| | | GAAAAGCCCCAUCCUUAGGCCUCCUCCUUCCUAGUC | |
| | | UCCUGAUAUUGGGUCUAACCCCCAGCAUAAUGAGUU | |
| | | GUCACACGCUCACAUGAGGAUCACCCAUGUGAU | |
| R1262_nc-9 | R1262 ncRNA containing | GGGAUAAUGAGCGUGUGACAAGAUUUUGGGCUUGU | SEQ ID NO: |
| (R1262_ncRNA- | 305 bp insertion in EMX1 | GUUUCGCAAGCUUUGAUUAAAAUGGUAGAUGGAUU | 19328 |
| EMX1- | site and MS2 stem loop at | UGCUAUCUAUCGUCAUUUCCAGUACUGUUAUGUACA | |
| MS2_305) | 3′ end | AACGGCAGAAGCUGGAGGAGGAAGGGCCUGAGUCCG | |
| | | AGCAGAAGAACGACUACCAAAUCCGCAUGUUACGGG | |
| | | ACUUCUUAUUAAUUCUUUUUUCGUGAGGAGCAGCGG | |
| | | AUCUUAAUGGAUGGCCGCAGGUGGUAUGGAAGCUAA | |
| | | UAGCGCGGGUGAGAGGGUAAUCAGCCGUGUCCACCA | |
| | | ACACAACGCUAUCGGGCGAUUCUAUAAGAUUCCGCA | |
| | | UUGCGUCUACUUAUAAGAUGUCUCAACGGUAUCCGC | |
| | | AACUUGCGAUGUGCCUGCUAUCCUUAAAUGCAUAUC | |
| | | UCGCCCAGUAGCUUCCCAAUAUGAGAGCAUCAAUUG | |
| | | UAGAUCGGGCCGGGAUAGUCAUGUCGUAAAGUUCUC | |
| | | CCAUCACAUCAACCGGUGGCGCAUUGCCACGAAGCA | |
| | | GGCCAAUGGGGAGGACAUCGAUGUCAGCAUAAUGAG | |
| | | UUGUCACACGCUCACAUGAGGAUCACCCAUGUGAU | |
| R1262_nc-7 | R1262 ncRNA containing | GGGAUAAUGAGCGUGUGACAAGAUUUUGGGCUUGU | SEQ ID NO: |
| (R1262_ncRNA- | 25 bp insertion in EMX1 | GUUUCGCAAGCUUUGAUUAAAAUGGUAGAUGGAUU | 19329 |
| EMX1- | site and MS2 stem loop at | UGCUAUCUAUCGUCAUUUCCAGUACUGUUAUGUACA | |
| MS2_25) | 3′ end | AACGGCAGAAGCUGGAGGAGGAAGGGCCUGAGUCCG | |
| | | AGCAGAAGAAAACUCGAGCUCUGAAUGACUCCUAUA | |
| | | AAGUUCUCCCAUCACAUCAACCGGUGGCGCAUUGCC | |
| | | ACGAAGCAGGCCAAUGGGGAGGACAUCGAUGUCAGC | |
| | | AUAAUGAGUUGUCACACGCUCACAUGAGGAUCACCC | |
| | | AUGUGAU | |
| R1262_nc-6 | R1262 ncRNA containing | GGGAUAAUGAGCGUGUGACAAGAUUUUGGGCUUGU | SEQ ID NO: |
| (R1262_ncRNA- | P2A-GFP insertion in | GUUUCGCAAGCUUUGAUUAAAAUGGUAGAUGGAUU | 19330 |
| EMX1_P2A- | EMX1 site | UGCUAUCUAUCGUCAUUUCCAGUACUGUUAUGUACA | |
| GFP) | | AACGGCAGAAGCUGGAGGAGGAAGGGCCUGAGUCCG | |
| | | AGCAGAAGAAGGAAGCGGAGCCACUAACUUCUCCCU | |
| | | GUUGAAACAAGCAGGGGAUGUCGAAGAGAAUCCCGG | |
| | | GCCAGUGAGCAAGGGCGAGGAGCUGUUCACCGGGGU | |
| | | GGUGCCCAUCCUGGUCGAGCUGGACGGCGACGUAAA | |
| | | CGGCCACAAGUUCAGCGUGUCCGGCGAGGGCGAGGG | |
| | | CGAUGCCACCUACGGCAAGCUGACCCUGAAGUUCAU | |
| | | CUGCACCACCGGCAAGCUGCCCGUGCCCUGGCCCACC | |
| | | CUCGUGACCACCCUGACCUACGGCGUGCAGUGCUUC | |
| | | AGCCGCUACCCCGACCACAUGAAGCAGCACGACUUC | |
| | | UUCAAGUCCGCCAUGCCCGAAGGCUACGUCCAGGAG | |
| | | CGCACCAUCUUCUUCAAGGACGACGGCAACUACAAG | |
| | | ACCCGCGCCGAGGUGAAGUUCGAGGGCGACACCCUG | |
| | | GUGAACCGCAUCGAGCUGAAGGGCAUCGACUUCAAG | |
| | | GAGGACGGCAACAUCCUGGGGCACAAGCUGGAGUAC | |
| | | AACUACAACAGCCACAACGUCUAUAUCAUGGCCGAC | |
| | | AAGCAGAAGAACGGCAUCAAGGUGAACUUCAAGAUC | |
| | | CGCCACAACAUCGAGGACGGCAGCGUGCAGCUCGCC | |
| | | GACCACUACCAGCAGAACACCCCCAUCGGCGACGGCC | |
| | | CCGUGCUGCUGCCCGACAACCACUACCUGAGCACCCA | |
| | | GUCCGCCCUGAGCAAAGACCCCAACGAGAAGCGCGA | |
| | | UCACAUGGUCCUGCUGGAGUUCGUGACCGCCGCCGG | |
| | | GAUCACUCUCGGCAUGGACGAGCUGUACAAGUAGAA | |
| | | AGUUCUCCCAUCACAUCAACCGGUGGCGCAUUGCCA | |
| | | CGAAGCAGGCCAAUGGGGAGGACAUCGAUGUCAGCA | |
| | | UAAUGAGUUGUCACACGCUCGAU | |
| R1262_nc-22 | R1262 ncRNA containing | GGGAUAAUGGGGCCACUAGGGACAGGAUGUUUCAGA | SEQ ID NO: |
| (R1262_ | 205 bp insertion in AAVS1 | GCUAUGCUGGAAACAGCAUAGCAAGUUGAAAUAAGG | 19331 |
| sgAAVS1- | site combined with | CUAGUCCGUUAUCAACUUGAAAAAGUGGCACCGAGU | |
| ncRNA_205) | sgAAVS1 at 5′ end | CGGUGCUUUUUAAUAACAACGAGCGUGUGACAAGAU | |
| | | UUUGGGCUUGUGUUUCGCAAGCUUUGAUUAAAAUGG | |
| | | UAGAUGGAUUUGCUAUCUAUCGUCAUUUCCAGUACU | |
| | | GUUAUGUUCUGUCCCCUCCACCCCACAGUGGGGCCA | |
| | | CUAGGGACAGAAAGCGGUCUUACGGUCAGUCGUAUU | |
| | | CCUUCUCGAGUUCCGUCCAGUUGAGCGUGUCACUCC | |
| | | CAGUGUACCUGCAAGCCGAGAUGGCUGUGCUUGGAG | |
| | | UCAAUCGCAUGUAGGAUGGUCUCCAGACACCGGGGC | |
| | | ACCAGUUUUCACGCCUAAAGCAUAAACGACGAGCAG | |
| | | UCAUGAAAGUCUUAGUACUGGACGUGCCGUUUCACA | |
| | | AUUAAUGACAGAAAAGCCCCAUCCUUAGGCCUCCUC | |
| | | CUUCCUAGUCUCCUGAUAUUGGGUCUAACCCCCAGC | |
| | | AUAAUGAGUUGUCACACGCUCGAU | |
| R1262_nc-8 | R1262 ncRNA containing | GGGAUAAUGAGCGUGUGACAAGAUUUUGGGCUUGU | SEQ ID NO: |
| (R1262_ncRNA- | 205 bp insertion in EMX1 | GUUUCGCAAGCUUUGAUUAAAAUGGUAGAUGGAUU | 19332 |
| EMX1- | site and MS2 stem loop at | UGCUAUCUAUCGUCAUUUCCAGUACUGUUAUGUACA | |
| MS2_205) | 3′ end | AACGGCAGAAGCUGGAGGAGGAAGGGCCUGAGUCCG | |
| | | AGCAGAAGAAAAAGCGGUCUUACGGUCAGUCGUAUU | |
| | | CCUUCUCGAGUUCCGUCCAGUUGAGCGUGUCACUCC | |
| | | CAGUGUACCUGCAAGCCGAGAUGGCUGUGCUUGGAG | |
| | | UCAAUCGCAUGUAGGAUGGUCUCCAGACACCGGGGC | |
| | | ACCAGUUUUCACGCCUAAAGCAUAAACGACGAGCAG | |
| | | UCAUGAAAGUCUUAGUACUGGACGUGCCGUUUCACA | |
| | | AAGUUCUCCCAUCACAUCAACCGGUGGCGCAUUGCC | |
| | | ACGAAGCAGGCCAAUGGGGAGGACAUCGAUGUCAGC | |
| | | AUAAUGAGUUGUCACACGCUCACAUGAGGAUCACCC | |
| | | AUGUGAU | |
| R1262_nc-21 | R1262 ncRNA containing | GGGAUAAUGAGCGUGUGACAAGAUUUUGGGCUUGU | SEQ ID NO: |
| (R1262_ncRNA- | 205 bp insertion in AAVS1 | GUUUCGCAAGCUUUGAUUAAAAUGGUAGAUGGAUU | 19333 |
| sgAAVS1_205) | site combined with | UGCUAUCUAUCGUCAUUUCCAGUACUGUUAUGUUCU | |
| | sgAAVS1 at 3′ end | GUCCCCUCCACCCCACAGUGGGGCCACUAGGGACAG | |
| | | AAAGCGGUCUUACGGUCAGUCGUAUUCCUUCUCGAG | |
| | | UUCCGUCCAGUUGAGCGUGUCACUCCCAGUGUACCU | |
| | | GCAAGCCGAGAUGGCUGUGCUUGGAGUCAAUCGCAU | |
| | | GUAGGAUGGUCUCCAGACACCGGGGCACCAGUUUUC | |
| | | ACGCCUAAAGCAUAAACGACGAGCAGUCAUGAAAGU | |
| | | CUUAGUACUGGACGUGCCGUUUCACAAUUAAUGACA | |
| | | GAAAAGCCCCAUCCUUAGGCCUCCUCCUUCCUAGUC | |
| | | UCCUGAUAUUGGGUCUAACCCCCAGCAUAAUGAGUU | |
| | | GUCACACGCUCAAUAACAACGGGGCCACUAGGGACA | |
| | | GGAUGUUUCAGAGCUAUGCUGGAAACAGCAUAGCAA | |
| | | GUUGAAAUAAGGCUAGUCCGUUAUCAACUUGAAAAA | |
| | | GUGGCACCGAGUCGGUGCUUUUUGAU | |
| R1262_nc-10 | R1262 ncRNA containing | GGGAUAAUGAGCGUGUGACAAGAUUUUGGGCUUGU | SEQ ID NO: |
| (R1262_ncRNA- | 405 bp insertion in EMX1 | GUUUCGCAAGCUUUGAUUAAAAUGGUAGAUGGAUU | 19334 |
| EMX1- | site and MS2 stem loop at | UGCUAUCUAUCGUCAUUUCCAGUACUGUUAUGUACA | |
| MS2_405) | 3′ end | AACGGCAGAAGCUGGAGGAGGAAGGGCCUGAGUCCG | |
| | | AGCAGAAGAACUGCUAAAUCCGCGUGAUAGGGGAUU | |
| | | UGAAGUUUAAUCUUCUAUCGCAAGGAACUGCCGAUC | |
| | | UUAAUGGAUGGCCGGAGGUGGUAUGGAAGCUAUAA | |
| | | GCGCGGGUGAGAGGGUAAUUAGGCGUGUUCACCUAC | |
| | | GCUACGCUAACGGGCGAUUCUAUAAGAUUGCACAUU | |
| | | GCGUCAACUCAUAAGAUGUCUCAACGGCAUGCGCAA | |
| | | CUUGUGAAGUGUCUACUAUCCUUAAACGCAUAUCUC | |
| | | GCACAGUAACUCCCGAAUAUGUCGGCAUCUGAUGUU | |
| | | GCCCGGGCCGAGUUAGUGUUGAGCUCACGGAACUUA | |
| | | UUGUAUGAGUAGUGAUUUGUAAGAGUUGUCAGUUA | |
| | | GCUCGUUCAGGUAAUAGUUGCCCACACAACGUCAAA | |
| | | AUAAGAGAACGGUCGUAACAUAAAGUUCUCCCAUCA | |
| | | CAUCAACCGGUGGCGCAUUGCCACGAAGCAGGCCAA | |
| | | UGGGGAGGACAUCGAUGUCAGCAUAAUGAGUUGUCA | |
| | | CACGCUCACAUGAGGAUCACCCAUGUGAU | |
| R1262_nc-11 | R1262 ncRNA containing | GGGAUAAUGAGCGUGUGACAAGAUUUUGGGCUUGU | SEQ ID NO: |
| (R1262_ncRNA- | 505 bp insertion in EMX1 | GUUUCGCAAGCUUUGAUUAAAAUGGUAGAUGGAUU | 19335 |
| EMX1- | site and MS2 stem loop at | UGCUAUCUAUCGUCAUUUCCAGUACUGUUAUGUACA | |
| MS2_505) | 3′ end | AACGGCAGAAGCUGGAGGAGGAAGGGCCUGAGUCCG | |
| | | AGCAGAAGAAACUUCAGCUAAGGAAGCUACCAAUAU | |
| | | UUAGUUUCUGAGUCUCACGACAGACCUCGCGCGUAG | |
| | | AUUGCCAUGCGUAGAGCUAACGAGCCAGCGGAAAGC | |
| | | GUGAGGCGCUUUUAAGCAUGGCGAGUAAGUGAUCCA | |
| | | ACGCUUCGGAUAUGACUAUAUACUUAGGUUCGAUCU | |
| | | CGUCCCGAGAAUUCUAAGCCUCAACAUCUAUGAGUU | |
| | | AUGAGGUUAGCCGAAAAAGCACGUGGUGGCGCCCAC | |
| | | CGACUGUUCCCAGACUGUAGCUCUUUGUUCUGUCAA | |
| | | GGCCCGACCUUCAUCGCGGCCGAUUCCUUCUGCGGA | |
| | | CCAUACCGUCCUGAUACUUUGGUCAUGUUUCCGUUG | |
| | | UAGGAGUGAACCCACUUGCCUUUGCGUCUUAAUACC | |
| | | AAUGAAAAACCUAUGCACUUUGUACAGGGUACCAUC | |
| | | GGGAUUCUGAACCCUCAGAUAGUGGGGAUCCCGGGU | |
| | | AUAGACCUUUAUCUGCGGUCCAACUUAGGCAUAAAC | |
| | | CUCCAUGCUACAAAGUUCUCCCAUCACAUCAACCGG | |
| | | UGGCGCAUUGCCACGAAGCAGGCCAAUGGGGAGGAC | |
| | | AUCGAUGUCAGCAUAAUGAGUUGUCACACGCUCACA | |
| | | UGAGGAUCACCCAUGUGAU | |
| R1262_nc-12 | R1262 ncRNA containing | GGGAUAAUGAGCGUGUGACAAGAUUUUGGGCUUGU | SEQ ID NO: |
| (R1262_ncRNA- | P2A-GFP insertion in | GUUUCGCAAGCUUUGAUUAAAAUGGUAGAUGGAUU | 19336 |
| EMX1- | EMX1 site and MS2 stem | UGCUAUCUAUCGUCAUUUCCAGUACUGUUAUGUACA | |
| MS2_P2A- | loop at 3′ cnd | AACGGCAGAAGCUGGAGGAGGAAGGGCCUGAGUCCG | |
| GFP) | | AGCAGAAGAAGGAAGCGGAGCCACUAACUUCUCCCU | |
| | | GUUGAAACAAGCAGGGGAUGUCGAAGAGAAUCCCGG | |
| | | GCCAGUGAGCAAGGGCGAGGAGCUGUUCACCGGGGU | |
| | | GGUGCCCAUCCUGGUCGAGCUGGACGGCGACGUAAA | |
| | | CGGCCACAAGUUCAGCGUGUCCGGCGAGGGCGAGGG | |
| | | CGAUGCCACCUACGGCAAGCUGACCCUGAAGUUCAU | |
| | | CUGCACCACCGGCAAGCUGCCCGUGCCCUGGCCCACC | |
| | | CUCGUGACCACCCUGACCUACGGCGUGCAGUGCUUC | |
| | | AGCCGCUACCCCGACCACAUGAAGCAGCACGACUUC | |
| | | UUCAAGUCCGCCAUGCCCGAAGGCUACGUCCAGGAG | |
| | | CGCACCAUCUUCUUCAAGGACGACGGCAACUACAAG | |
| | | ACCCGCGCCGAGGUGAAGUUCGAGGGCGACACCCUG | |
| | | GUGAACCGCAUCGAGCUGAAGGGCAUCGACUUCAAG | |
| | | GAGGACGGCAACAUCCUGGGGCACAAGCUGGAGUAC | |
| | | AACUACAACAGCCACAACGUCUAUAUCAUGGCCGAC | |
| | | AAGCAGAAGAACGGCAUCAAGGUGAACUUCAAGAUC | |
| | | CGCCACAACAUCGAGGACGGCAGCGUGCAGCUCGCC | |
| | | GACCACUACCAGCAGAACACCCCCAUCGGCGACGGCC | |
| | | CCGUGCUGCUGCCCGACAACCACUACCUGAGCACCCA | |
| | | GUCCGCCCUGAGCAAAGACCCCAACGAGAAGCGCGA | |
| | | UCACAUGGUCCUGCUGGAGUUCGUGACCGCCGCCGG | |
| | | GAUCACUCUCGGCAUGGACGAGCUGUACAAGUAGAA | |
| | | AGUUCUCCCAUCACAUCAACCGGUGGCGCAUUGCCA | |
| | | CGAAGCAGGCCAAUGGGGAGGACAUCGAUGUCAGCA | |
| | | UAAUGAGUUGUCACACGCUCACAUGAGGAUCACCCA | |
| | | UGUGAU | |
| R1262_nc-23 | Same as R1262_nc-6 but | GGGAUAAUGAGCGUGUGACAAGAUUUUGGGCUUGU | SEQ ID NO: |
| (R1262_ncRNA- | with longer homology arms | GUUUCGCAAGCUUUGAUUAAAAUGGUAGAUGGAUU | 19337 |
| EMX1_P2A- | | UGCUAUCUAUCGUCAUUUCCAGUACUGUUAUGUGAG | |
| GFP_LongHA) | | GCCCCAGUGGCUGCUCUGGGGGCCUCCUGAGUUUCU | |
| | | CAUCUGUGCCCCUCCCUCCCUGGCCCAGGUGAAGGU | |
| | | GUGGUUCCAGAACCGGAGGACAAAGUACAAACGGCA | |
| | | GAAGCUGGAGGAGGAAGGGCCUGAGUCCGAGCAGAA | |
| | | GAAGGAAGCGGAGCCACUAACUUCUCCCUGUUGAAA | |
| | | CAAGCAGGGGAUGUCGAAGAGAAUCCCGGGCCAGUG | |
| | | AGCAAGGGCGAGGAGCUGUUCACCGGGGUGGUGCCC | |
| | | AUCCUGGUCGAGCUGGACGGCGACGUAAACGGCCAC | |
| | | AAGUUCAGCGUGUCCGGCGAGGGCGAGGGCGAUGCC | |
| | | ACCUACGGCAAGCUGACCCUGAAGUUCAUCUGCACC | |
| | | ACCGGCAAGCUGCCCGUGCCCUGGCCCACCCUCGUGA | |
| | | CCACCCUGACCUACGGCGUGCAGUGCUUCAGCCGCU | |
| | | ACCCCGACCACAUGAAGCAGCACGACUUCUUCAAGU | |
| | | CCGCCAUGCCCGAAGGCUACGUCCAGGAGCGCACCA | |
| | | UCUUCUUCAAGGACGACGGCAACUACAAGACCCGCG | |
| | | CCGAGGUGAAGUUCGAGGGCGACACCCUGGUGAACC | |
| | | GCAUCGAGCUGAAGGGCAUCGACUUCAAGGAGGACG | |
| | | GCAACAUCCUGGGGCACAAGCUGGAGUACAACUACA | |
| | | ACAGCCACAACGUCUAUAUCAUGGCCGACAAGCAGA | |
| | | AGAACGGCAUCAAGGUGAACUUCAAGAUCCGCCACA | |
| | | ACAUCGAGGACGGCAGCGUGCAGCUCGCCGACCACU | |
| | | ACCAGCAGAACACCCCCAUCGGCGACGGCCCCGUGCU | |
| | | GCUGCCCGACAACCACUACCUGAGCACCCAGUCCGCC | |
| | | CUGAGCAAAGACCCCAACGAGAAGCGCGAUCACAUG | |
| | | GUCCUGCUGGAGUUCGUGACCGCCGCCGGGAUCACU | |
| | | CUCGGCAUGGACGAGCUGUACAAGUAGAAAGUUCUC | |
| | | CCAUCACAUCAACCGGUGGCGCAUUGCCACGAAGCA | |
| | | GGCCAAUGGGGAGGACAUCGAUGUCACCUCCAAUGA | |
| | | CUAGGGUGGGCAACCACAAACCCACGAGGGCAGAGU | |
| | | GCUGCUUGCUGCUGGCCAGGCCCCUGCGUGGGCCCA | |
| | | AGCGCAUAAUGAGUUGUCACACGCUCGAU | |
| R1262_nc-24 | Same as R1262_nc-12 but | GGGAUAAUGAGCGUGUGACAAGAUUUUGGGCUUGU | SEQ ID NO: |
| (R1262_ncRNA- | with longer homology arms | GUUUCGCAAGCUUUGAUUAAAAUGGUAGAUGGAUU | 19338 |
| EMX1- | | UGCUAUCUAUCGUCAUUUCCAGUACUGUUAUGUGAG | |
| MS2_P2A- | | GCCCCAGUGGCUGCUCUGGGGGCCUCCUGAGUUUCU | |
| GFP_LongHA) | | CAUCUGUGCCCCUCCCUCCCUGGCCCAGGUGAAGGU | |
| | | GUGGUUCCAGAACCGGAGGACAAAGUACAAACGGCA | |
| | | GAAGCUGGAGGAGGAAGGGCCUGAGUCCGAGCAGAA | |
| | | GAAGGAAGCGGAGCCACUAACUUCUCCCUGUUGAAA | |
| | | CAAGCAGGGGAUGUCGAAGAGAAUCCCGGGCCAGUG | |
| | | AGCAAGGGCGAGGAGCUGUUCACCGGGGUGGUGCCC | |
| | | AUCCUGGUCGAGCUGGACGGCGACGUAAACGGCCAC | |
| | | AAGUUCAGCGUGUCCGGCGAGGGCGAGGGCGAUGCC | |
| | | ACCUACGGCAAGCUGACCCUGAAGUUCAUCUGCACC | |
| | | ACCGGCAAGCUGCCCGUGCCCUGGCCCACCCUCGUGA | |
| | | CCACCCUGACCUACGGCGUGCAGUGCUUCAGCCGCU | |
| | | ACCCCGACCACAUGAAGCAGCACGACUUCUUCAAGU | |
| | | CCGCCAUGCCCGAAGGCUACGUCCAGGAGCGCACCA | |
| | | UCUUCUUCAAGGACGACGGCAACUACAAGACCCGCG | |
| | | CCGAGGUGAAGUUCGAGGGCGACACCCUGGUGAACC | |
| | | GCAUCGAGCUGAAGGGCAUCGACUUCAAGGAGGACG | |
| | | GCAACAUCCUGGGGCACAAGCUGGAGUACAACUACA | |
| | | ACAGCCACAACGUCUAUAUCAUGGCCGACAAGCAGA | |
| | | AGAACGGCAUCAAGGUGAACUUCAAGAUCCGCCACA | |
| | | ACAUCGAGGACGGCAGCGUGCAGCUCGCCGACCACU | |
| | | ACCAGCAGAACACCCCCAUCGGCGACGGCCCCGUGCU | |
| | | GCUGCCCGACAACCACUACCUGAGCACCCAGUCCGCC | |
| | | CUGAGCAAAGACCCCAACGAGAAGCGCGAUCACAUG | |
| | | GUCCUGCUGGAGUUCGUGACCGCCGCCGGGAUCACU | |
| | | CUCGGCAUGGACGAGCUGUACAAGUAGAAAGUUCUC | |
| | | CCAUCACAUCAACCGGUGGCGCAUUGCCACGAAGCA | |
| | | GGCCAAUGGGGAGGACAUCGAUGUCACCUCCAAUGA | |
| | | CUAGGGUGGGCAACCACAAACCCACGAGGGCAGAGU | |
| | | GCUGCUUGCUGCUGGCCAGGCCCCUGCGUGGGCCCA | |
| | | AGCGCAUAAUGAGUUGUCACACGCUCACAUGAGGAU | |
| | | CACCCAUGUGAU | |
| 6083 v1 RT | 6083 retron RT mRNA | AUGAGCAACCCUCAGCCCACCAGGGCUGAGAUCUUC | SEQ ID NO: |
| | | GAGCGCAUCAAACAGAGCUCGAAGCAGGAGGUGAUC | 19339 |
| | | CUGGAGGAGAUGCAGAGACUGGGCUUCUGGCCAAGG | |
| | | UCAGAAGGCCAGCCUGAGGUCGCUGCCGACCUCAUC | |
| | | CAGAGAGAAGGCGAGCUCCAAAGGGAGCUCGCCGAA | |
| | | CUCAACAAGAAGUUGGCGGUCAAACGCAACCCCGAG | |
| | | AGGGCCCUCAGAGAAAUGAGGAAGCAGAGAAUGAAG | |
| | | GACGCCAGAGACAAGAGAGAGGUGACCAAGAGAGCC | |
| | | CAGGCUCAGCAGAGAUACGACAAGGCCCUGCUGUGG | |
| | | CACGAGAAGAGGGCCAGCCACGUGGCCUAUCUUGGC | |
| | | CCUGGUGUGAGCGCCAGCUUACACGAGAACAGCUCU | |
| | | GCUACCCAGGAGCAGGGCGACAAGGGGAAACCCAAG | |
| | | AGAGCCCGAGAUCGGGCUGUGCCAGACCUGCAGAGG | |
| | | CUGACACUGAACGGGCUGCCUGCUCUGAUUAGCGCA | |
| | | GCCCAACUCGCUGAGUCCAUGGGAGUCAGCGUGGCU | |
| | | GAGCUGAGAUUCCUCAGCUUCCACAGGGAGGUGGCU | |
| | | AGGACAAACCACUACCACAGCUUCACGCUCCCCAAG | |
| | | AAGACAGGUGGGGAGCGUCUGAUCAGCGCCCCCAUG | |
| | | CCCAGACUGAAGAGAGCCCAGUACUGGGUGCUGGAC | |
| | | AACGUGCUGGCAAAGAUGCCAGCGCACGAUGCGGCU | |
| | | CACGGCUUCCUGGCCGGCAGAAGCAUCAUCAGCAAU | |
| | | GCCAAGCCCCAUGCGGGGCAGGAUGUGGUCAUUAAC | |
| | | UUGGACGUUAAGGACUUCUUCCCCAGCAUCGCCUUC | |
| | | GGCAGAAUCAAGGGCGUGUUCAGACAGCUGGGCUAC | |
| | | GGCGAAAGCAUCGCCACAGUGUUCGCCCUGCUGUGC | |
| | | AGCGAGAACAGAGCCCAGGCCUGGCAAGUGGACGGA | |
| | | GAGAGACUCUUCGUCGGCGGCAAGGCCAGAGAAAGA | |
| | | GUGCUGCCUCAGGGCGCUCCUACCAGCCCCAUGCUG | |
| | | ACCAACCUGCUGUGCCGGCGGAUGGAUAGACGUCUG | |
| | | CUGGGUCUCGCGAAGCAGCUGGGCUUCGUGUACACC | |
| | | AGAUACGCCGACGACCUGACCUUCAGCGCCUCCGGC | |
| | | GAACCCGCAAGGGAUAACGUCGGAAAGCUGCUGAGC | |
| | | AGGGUCCGGUGGAUCCUGAGGGAUGAGGGGUUCACC | |
| | | CCUCAUCCCGAUAAGGAGAGAGUGAUGAGAAAGGGC | |
| | | AGAAGACAGGAGGUGACCGGCCUUGUGGUCAACUCC | |
| | | GACACUCCCAGCGUGAGCAGGGAGACCAGAAGAAGG | |
| | | CUGAGAGCCGCUCUGCACAGAGCCUCGCAGCCUGAC | |
| | | GCUGCGAGCAAACCUGCACAUUGGCAGGGCCAUACG | |
| | | GCCCAGCCAAGUCAGCUGCUGGGCCUGGCCACAUUC | |
| | | GUGCAUCAGAUCGACCCCAAGCAGGGCAAGACCCUG | |
| | | CUCGCUGACGCUCAGCAACUGAUGCGCAGCCCUAUC | |
| | | GACCGCGCUAAUGACGCGGCGAAGUCUGCUAGCAGG | |
| | | GCUGACGCUGCUCAGCAGAGCUUCAGAGUGCUGGCU | |
| | | GCUGCCGGCAAGCCACCAGUUCUUGCCGACGGCAAG | |
| | | AACUGGUGGCAACCUGCUCCUCCUGCCACACCUGUG | |
| | | CUGGAGAAGACCGACCAGCAGAGAAGAGAGGAAAGG | |
| | | CAGGCUACCAGAAGGCAGCAGGCCGCUGCUGCUGCU | |
| | | CCUCCUCCUAGCAGCACAAGAAGAAACGAGCGGCCG | |
| | | CAACAGGCCGCUCAUGAGCAGCAGGGAGAUGCCCAG | |
| | | CCUCAGAACGAGGCCCCUCCUAGAUUCGACCCCGACC | |
| | | AGUACGCUCCUCCCCCGAGGAACGUGAUGACCUACU | |
| | | GGGCCCAGAUCGCCAUCAGCUUCUUUCUGGGCAGCA | |
| | | UCCUGCACAACAGACUGAUCACGAUCUUCGCCAUGG | |
| | | UGGCGGUGAUCGCUCUGUACUACAUGAGAAGACAGA | |
| | | GAUGGGAUGUCUUCAUGGGCAUCCUGGUGGUGGCCA | |
| | | CCCUGCUGGGAUACCUGGUCAGGGGCAUGGGCccaccaa | |
| | | agaagaaaagaaaggucuga | |
| 6083 | R6083 retron containing 25 | GCUCCGGAGCAAUGAGCAGGCUCUUGCAAUCCGGGC | SEQ ID NO: |
| v1_ncRNA_ | bp insertion at AAVS1 site | GGUGUUUCGCCGCCCUUGUGAACUGCCGUUUCAUGC | 19340 |
| AAVS1 | | ACCACGGGCGCCGUUUUCACUGUGCCACCCCAGCCAC | |
| gRNA_25 bp | | GGUAGUGCUUCUGUCCCCUCCACCCCACAGUGGGGC | |
| | | CACUAGGGACAGAAAUGACAGUGGUUGGUGCUCUAA | |
| | | AAAUUAAUGACAGAAAAGCCCCAUCCUUAGGCCUCC | |
| | | UCCUUCCUAGUCUCCUGAUAUUGGGUCUAACCCCCA | |
| | | AGCACUACCGUGGCGGGGUGGCGUCGAGCGAACAGC | |
| | | UCCCGUCCCCUGAGCCCUACAGGCUCUUGGACGAGA | |
| | | UGCACAUUGCUCCGGAGCAAUAACAACGGGGCCACU | |
| | | AGGGACAGGAUguuucagagcuaugcuggaaacagcauagcaaguuga | |
| | | aauaaggcuaguccguuaucaacuugaaaaaguggcaccgagucggugcuuuuu | |
| 6083 | R6083 retron containing 25 | GCUCCGGAGCAAUGAGCAGGCUCUUGCAAUCCGGGC | SEQ ID NO: |
| v1_ncRNA | bp insertion at EMX1 site | GGUGUUUCGCCGCCCUUGUGAACUGCCGUUUCAUGC | 19341 |
| only_25 bp | | ACCACGGGCGCCGUUUUCACUGUGCCACCCCAGCCAC | |
| | | GGUAGUGCUacaaacggcagaagcuggaggaggaagggccugaguccgag | |
| | | cagaagaaaACUCGAGCUCUGAAUGACUCCUAU Aaaguucuc | |
| | | ccaucacaucaaccgguggcgcauugccacgaagcaggccaauggggaggacauc | |
| | | gaugucaAGCACUACCGUGGCGGGGUGGCGUCGAGCGA | |
| | | ACAGCUCCCGUCCCCUGAGCCCUACAGGCUCUUGGA | |
| | | CGAGAUGCACAUUGCUCCGGAGC | |
| 6083 | R6083 retron containing 25 | GCUCCGGAGCAAUGAGCAGGCUCUUGCAAUCCGGGC | SEQ ID NO: |
| v1_ncRNA_ | bp insertion at EMX1 site | GGUGUUUCGCCGCCCUUGUGAACUGCCGUUUCAUGC | 19342 |
| EMX1 | and sgEMX1 at 3′ end | ACCACGGGCGCCGUUUUCACUGUGCCACCCCAGCCAC | |
| gRNA_25 bp | | GGUAGUGCUacaaacggcagaagcuggaggaggaagggccugaguccgag | |
| | | cagaagaaaACUCGAGCUCUGAAUGACUCCUAUAaaguucuc | |
| | | ccaucacaucaaccgguggcgcauugccacgaagcaggccaauggggaggacauc | |
| | | gaugucaAGCACUACCGUGGCGGGGUGGCGUCGAGCGA | |
| | | ACAGCUCCCGUCCCCUGAGCCCUACAGGCUCUUGGA | |
| | | CGAGAUGCACAUUGCUCCGGAGCAAUAACAACgagucc | |
| | | gagcagaagaagaaguuucagagcuaugcuggaaacagcauagcaaguugaaau | |
| | | aaggcuaguccguuaucaacuugaaaaaguggcaccgagucggugcuuuuu | |
| 6083 | R6083 retron containing | GCUCCGGAGCAAUGAGCAGGCUCUUGCAAUCCGGGC | SEQ ID NO: |
| v1_ncRNA_ | GFP gene insertion at | GGUGUUUCGCCGCCCUUGUGAACUGCCGUUUCAUGC | 19343 |
| EMX1 | EMX1 site and sgEMX1 at | ACCACGGGCGCCGUUUUCACUGUGCCACCCCAGCCAC | |
| gRNA_GFP | 3′ end | GGUAGUGCUacaaacggcagaagcuggaggaggaagggccugaguccgag | |
| gene | | cagaagaacccauauguccuuccgagugagagacacaaaaaauuccaacacacua | |
| | | uugcaaugaaaauaaauuuccuuuauuagccagaagucagaugcucaaggggcu | |
| | | ucaugauguccccauaauuuuggcagagggaaaaagaucucagugguauuugu | |
| | | gagccagggcauuggccacaccagccaccaccuucugauaggcagccugcaccug | |
| | | aggagugcggccgcuuuacuuguacagcucguccaugccgagagugaucccggc | |
| | | ggcggucacgaacuccagcaggaccaugugaucgcgcuucucguuggggucuuu | |
| | | gcucagggggacugggugcucagguagugguugucgggcagcagcacggggc | |
| | | cgucgccgauggggguguucugcugguaguggucggcgagcugcacgcugccg | |
| | | uccucgauguuguggcggaucuugaaguucaccuugaugccguucuucugcuu | |
| | | gucggccaugauauagacguuguggcuguuguaguuguacuccagcuugugcc | |
| | | ccaggauguugccguccuccuugaagucgaugcccuucagcucgaugcgguuca | |
| | | ccagggugucgcccucgaacuucaccucggcgcgggucuuguaguugccgucgu | |
| | | ccuugaagaagauggugcgcuccuggacguagccuucgggcauggeggacuuga | |
| | | agaagucgugcugcuucauguggucgggguageggcugaagcacugcacgccgu | |
| | | aggucaggguggucacgagggugggccagggcacgggcagcuugccgguggug | |
| | | cagaugaacuucagggucagcuugccguagguggcaucgcccucgcccucgccg | |
| | | gacacgcugaacuuguggccguuuacgucgccguccagcucgaccaggaugggc | |
| | | accaccccggugaacagcuccucgcccuugcucaccaugguggcgaccggugga | |
| | | ucccgggcccgegguaccgucgacugcagaauucgaagcuugagcucgagaucu | |
| | | gaguccgguagcgcuagcggaucugacgguucacuaaacccuguguucugggg | |
| | | caaacccguugcgaaaaagaacguucacggcgacuacugcacuuauauacgguuc | |
| | | ucccccacccucgggaaaaaggeggagccaguacacgacaucacuuucccaguuu | |
| | | accccgcgccaccuucucuaggcacegguucaauugccgaccccuccccccaacu | |
| | | ucucggggacugugggcgaugugegcucugcccguucucccaucacaucaaccg | |
| | | guggcgcauugccacgaagcaggccaauggggaggacaucgaugucaAGCAC | |
| | | UACCGUGGCGGGGUGGCGUCGAGCGAACAGCUCCCG | |
| | | UCCCCUGAGCCCUACAGGCUCUUGGACGAGAUGCAC | |
| | | AUUGCUCCGGAGCAAUAACAACgaguccgagcagaagaagaag | |
| | | uuucagagcuaugcuggaaacagcauagcaaguugaaauaaggcuaguccguua | |
| | | ucaacuugaaaaaguggcaccgagucggugcuuuuu | |
| 6083 | R6083 retron containing 50 | GCUCCGGAGCAAUGAGCAGGCUCUUGCAAUCCGGGC | SEQ ID NO: |
| v1_ncRNA_ | bp insertion at EMX1 site | GGUGUUUCGCCGCCCUUGUGAACUGCCGUUUCAUGC | 19344 |
| EMX1 | and sgEMX1 at 3′ end | ACCACGGGCGCCGUUUUCACUGUGCCACCCCAGCCAC | |
| gRNA_50 bp | | GGUAGUGCUacaaacggcagaagcuggaggaggaagggccugaguccgag | |
| | | cagaagaaaACUCGAGCUCUGAUCAGUAUCAUGGGGCCC | |
| | | AUGGUUGAAUGACUCCUAUAaaguucucccaucacaucaaccgg | |
| | | uggcgcauugccacgaagcaggccaauggggaggacaucgaugucaAGCAC | |
| | | UACCGUGGCGGGGUGGCGUCGAGCGAACAGCUCCCG | |
| | | UCCCCUGAGCCCUACAGGCUCUUGGACGAGAUGCAC | |
| | | AUUGCUCCGGAGCAAUAACAACgaguccgagcagaagaagaag | |
| | | uuucagagcuaugcuggaaacagcauagcaaguugaaauaaggcuaguccguua | |
| | | ucaacuugaaaaaguggcaccgagucggugcuuuuu | |
| 6083 | R6083 retron containing | GCUCCGGAGCAAUGAGCAGGCUCUUGCAAUCCGGGC | SEQ ID NO: |
| v1_ncRNA_ | 100 bp insertion at EMX1 | GGUGUUUCGCCGCCCUUGUGAACUGCCGUUUCAUGC | 19345 |
| EMX1 | site and sgEMX1 al 3′ end | ACCACGGGCGCCGUUUUCACUGUGCCACCCCAGCCAC | |
| gRNA_100 bp | | GGUAGUGCUacaaacggcagaagcuggaggaggaagggccugaguccgag | |
| | | cagaagaaaACUCGAGCUCUGAGCCCCACUGUCGAGAAG | |
| | | UAUGUAUCUCGCUCCCGCAGCUUGCCAGCACUUUCA | |
| | | GUAUCAUGGGGCCCAUGGUUGAAUGACUCCUAUAaag | |
| | | uucucccaucacaucaaccgguggcgcauugccacgaagcaggccaauggggag | |
| | | gacaucgaugucaAGCACUACCGUGGCGGGGUGGCGUCGA | |
| | | GCGAACAGCUCCCGUCCCCUGAGCCCUACAGGCUCU | |
| | | UGGACGAGAUGCACAUUGCUCCGGAGCAAUAACAAC | |
| | | gaguccgagcagaagaagaaguuucagagcuaugcuggaaacagcauagcaagu | |
| | | ugaaauaaggcuaguccguuaucaacuugaaaaaguggcaccgagucggugcuu | |
| | | uuu | |
| 6083 | R6083 retron containing 75 | GCUCCGGAGCAAUGAGCAGGCUCUUGCAAUCCGGGC | SEQ ID NO: |
| v1_ncRNA_ | bp insertion at EMX1 site | GGUGUUUCGCCGCCCUUGUGAACUGCCGUUUCAUGC | 19346 |
| EMX1 | and sgEMX1 at 3′ end | ACCACGGGCGCCGUUUUCACUGUGCCACCCCAGCCAC | |
| gRNA_75 bp | | GGUAGUGCUacaaacggcagaagcuggaggaggaagggccugaguccgag | |
| | | cagaagaaaACUCGAGCUCUGAGCCCCACCGCAGCUUGCC | |
| | | AGCACUUUCAGUAUCAUGGGGCCCAUGGUUGAAUGA | |
| | | CUCCUAUAaaguucucccaucacaucaaccgguggegcauugccacgaagc | |
| | | aggccaauggggaggacaucgaugucaAGCACUACCGUGGCGGGG | |
| | | UGGCGUCGAGCGAACAGCUCCCGUCCCCUGAGCCCU | |
| | | ACAGGCUCUUGGACGAGAUGCACAUUGCUCCGGAGC | |
| | | AAUAACAACgaguccgagcagaagaagaaguuucagagcuaugcuggaaa | |
| | | cagcauagcaaguugaaauaaggcuaguccguuaucaacuugaaaaaguggcacc | |
| | | gagucggugcuuuuu | |
| Aco1_ncRNA- | Aco1 ncRNA containing | GGGAUAAUGUAUAAAACCGGGAACGAUCAGACCGGG | SEQ ID NO: |
| EMX1_100 | 100 bp insertion in EMX1 | GUGAAUUCGCCCCCUUGAUCAAACGGCACUAACCAC | 19347 |
| | gene | UGUUUGCCGUGCGUGCGUACAAACGGCAGAAGCUGG | |
| | | AGGAGGAAGGGCCUGAGUCCGAGCAGAAGAAAACUC | |
| | | GAGCUCUGAGCCCCACUGUCGAGAAGUAUGUAUCUC | |
| | | GCUCCCGCAGCUUGCCAGCACUUUCAGUAUCAUGGG | |
| | | GCCCAUGGUUGAAUGACUCCUAUAAAGUUCUCCCAU | |
| | | CACAUCAACCGGUGGCGCAUUGCCACGAAGCAGGCC | |
| | | AAUGGGGAGGACAUCGAUGUCAACGCACGCACGGCA | |
| | | AACAGACAGAUCCAUUAUUAUUACAAUUUAUUUAGU | |
| | | GAUCGUUCCCGGUUUUAUACGAU | |
| Aco1_ncRNA- | Aco1 ncRNA containing | GGGAUAAUGUAUAAAACCGGGAACGAUCAGACCGGG | SEQ ID NO: |
| EMX1_205 | 205 bp insertion in EMX1 | GUGAAUUCGCCCCCUUGAUCAAACGGCACUAACCAC | 19348 |
| | gene | UGUUUGCCGUGCGUGCGUACAAACGGCAGAAGCUGG | |
| | | AGGAGGAAGGGCCUGAGUCCGAGCAGAAGAAAAAGC | |
| | | GGUCUUACGGUCAGUCGUAUUCCUUCUCGAGUUCCG | |
| | | UCCAGUUGAGCGUGUCACUCCCAGUGUACCUGCAAG | |
| | | CCGAGAUGGCUGUGCUUGGAGUCAAUCGCAUGUAGG | |
| | | AUGGUCUCCAGACACCGGGGCACCAGUUUUCACGCC | |
| | | UAAAGCAUAAACGACGAGCAGUCAUGAAAGUCUUAG | |
| | | UACUGGACGUGCCGUUUCACAAAGUUCUCCCAUCAC | |
| | | AUCAACCGGUGGCGCAUUGCCACGAAGCAGGCCAAU | |
| | | GGGGAGGACAUCGAUGUCAACGCACGCACGGCAAAC | |
| | | AGACAGAUCCAUUAUUAUUACAAUUUAUUUAGUGAU | |
| | | CGUUCCCGGUUUUAUACGAU | |
| Eco3_ncRNA- | Eco3 ncRNA containing | GGGAUAAUUGAUAAGAUUCCGUAAGAGCCAAACCUA | SEQ ID NO: |
| EMX1_205 | 205 bp insertion in EMX1 | GCAUUUUAUGGGUUAAUAGCCCAUCGGGCCAUGAGU | 19349 |
| | gene | CAUGGUUUCGCCUAGUAUUUUAGCUAUGCCCGUCGU | |
| | | UCAGUUCGCUGAACAAACGGCAGAAGCUGGAGGAGG | |
| | | AAGGGCCUGAGUCCGAGCAGAAGAAAAAGCGGUCUU | |
| | | ACGGUCAGUCGUAUUCCUUCUCGAGUUCCGUCCAGU | |
| | | UGAGCGUGUCACUCCCAGUGUACCUGCAAGCCGAGA | |
| | | UGGCUGUGCUUGGAGUCAAUCGCAUGUAGGAUGGUC | |
| | | UCCAGACACCGGGGCACCAGUUUUCACGCCUAAAGC | |
| | | AUAAACGACGAGCAGUCAUGAAAGUCUUAGUACUGG | |
| | | ACGUGCCGUUUCACAAAGUUCUCCCAUCACAUCAAC | |
| | | CGGUGGCGCAUUGCCACGAAGCAGGCCAAUGGGGAG | |
| | | GACAUCGAUGUCAUCAGCGAACUGAUCGACGUGCUC | |
| | | AAGUAGGUUUGGCUCUUACGGAAUCUUAUCAGAU | |
| R2781 RT-1 | R2781 retron RT | AGGAUAAUGGGAAAUAAGAGAGAAAAGAAGAGUAA | SEQ ID NO: |
| | | GAAGAAAUAUAAGAGGAUCCGCCACCAUGGGUUACA | 19350 |
| | | ACUACGAGUACACCAUCAGGCUGUGUGAGACGCUCA | |
| | | AGUCCUUGAGCGCUGACGACGUGUACAUCAGCAAGU | |
| | | GCUGCAACUACGCCGAGGGACUCCUGGAUAAGGAGC | |
| | | UCCCUGUGAUCUUCGACCCCACCCACCUGAAGCAGA | |
| | | UCCUGAGGCUGGACGACAUCAGCCUCGACGAGUACC | |
| | | ACAUCUUCUAUAUCGACAAGAAGAACGGGGGCAGCA | |
| | | GAGAGAUCAAUGCCCCCAGCGAGGAGCUGAAGAAGC | |
| | | GCCAACGCUGGAUUCUCAAGAACAUCCUUGAGAAGA | |
| | | UCAGCAUCAGCCACAACGUGCACGGCUUCAUCAAGG | |
| | | GCAAGAGCAUUGUCAGCAAUGCUCGCAAGCACCUGA | |
| | | ACAAGGAGUAUGUGCUGAACAUCGACAUCAAGGACU | |
| | | UCUUCCCCAGCGUCACCAAAUACUCCGUGGAGAAGA | |
| | | UCUUCCGGAGAAUGGGCUACUGCAACAGCGUGGCCC | |
| | | AGCUGCUUGCCCGCGUCUGUUGUUACAGAGGCGGGC | |
| | | UGCCUCAGGGAGCUCCUACAAGCCCCUACCUGGCCA | |
| | | ACCUCGCUUUUGACGAGGUUGACCAGGAGAUCAUCA | |
| | | ACGUGGUGAGAAACCGGGACAUCACCUACACCAGAU | |
| | | ACGCCGACGACAUGACCUUCAGCGCCAACUACGACC | |
| | | UGAGCACCUUCAAGAAGGAGGUGUACAAGAGCCUGG | |
| | | GCAAAUACAGAUUCAGCCCCAACAUCAUGAAGACUC | |
| | | ACCAGAUGUCUGGCGAGAAGAGAAAGCUGGUGACCG | |
| | | GCCUGAUCGUGGACGACAAGGUGAAGGUGUGCAAGA | |
| | | AGUACAAGAGAAAGCUGCGGCAGGAGAUCUACUACU | |
| | | GCAAGAAGUUCGGCGUGACCAACCACCUGAGAAACU | |
| | | GCCACAGCGAGAAGUCCAUCAACUACAAGGAGUACC | |
| | | UGUACGGCAAGGCCUACUUCAUCAAGAUGGUCGAGG | |
| | | AGAUCGUCGGCGAGAAGUUCCUGGCCGACCUGGACA | |
| | | GCAUUGACUGGUACCCACCAAAGAAGAAAAGAAAGG | |
| | | UCUGACUCGAGGCUGGAGCCUCGGUGGCCAUGCUUC | |
| | | UUGCCCCUUGGGCCUCCCCCCAGCCCCUCCUCCCCUU | |
| | | CCUGCACCCGUACCCCCGUGGUCUUUGAAUAAAGUC | |
| | | UGAGUGGGCGGC | |
| R2781_nc- | R2781 ncRNA containing | GGGAUAAUAAGAGCAACUAGAUUGAGGCGAUUCGCC | SEQ ID NO: |
| 01_25 bp_49- | 25 bp insertion in EMX1 | UCCUUGGAAAAGGGUACUAAGUUUCUGUCACAAACG | 19351 |
| 65HA | gene with 49 and 69 bp | GCAGAAGCUGGAGGAGGAAGGGCCUGAGUCCGAGCA | |
| | homology arms | GAAGAAAACUCGAGCUCUGAAUGACUCCUAUAAAGU | |
| | | UCUCCCAUCACAUCAACCGGUGGCGCAUUGCCACGA | |
| | | AGCAGGCCAAUGGGGAGGACAUCGAUGUCAGACAGA | |
| | | AAUGAAAUAAAUAGUAGUUGCUCUU | |
| R2781_nc- | R2781 ncRNA containing | GGGAUAAUAAGAGCAACUAGAUUGAGGCGAUUCGCC | SEQ ID NO: |
| 02_205 bp_49- | 205 bp insertion in EMX1 | UCCUUGGAAAAGGGUACUAAGUUUCUGUCACAAACG | 19352 |
| 65HA | gene with 49 and 69 bp | GCAGAAGCUGGAGGAGGAAGGGCCUGAGUCCGAGCA | |
| | homology arms | GAAGAAAAAGCGGUCUUACGGUCAGUCGUAUUCCUU | |
| | | CUCGAGUUCCGUCCAGUUGAGCGUGUCACUCCCAGU | |
| | | GUACCUGCAAGCCGAGAUGGCUGUGCUUGGAGUCAA | |
| | | UCGCAUGUAGGAUGGUCUCCAGACACCGGGGCACCA | |
| | | GUUUUCACGCCUAAAGCAUAAACGACGAGCAGUCAU | |
| | | GAAAGUCUUAGUACUGGACGUGCCGUUUCACAAAGU | |
| | | UCUCCCAUCACAUCAACCGGUGGCGCAUUGCCACGA | |
| | | AGCAGGCCAAUGGGGAGGACAUCGAUGUCAGACAGA | |
| | | AAUGAAAUAAAUAGUAGUUGCUCUU | |
| R2781_nc- | R2781 ncRNA containing | GGGAUAAUAAGAGCAACUAGAUUGAGGCGAUUCGCC | SEQ ID NO: |
| 03_405 bp_49- | 405 bp insertion in EMX1 | UCCUUGGAAAAGGGUACUAAGUUUCUGUCACAAACG | 19353 |
| 65HA | gene with 49 and 69 bp | GCAGAAGCUGGAGGAGGAAGGGCCUGAGUCCGAGCA | |
| | homology arms | GAAGAACUGCUAAAUCCGCGUGAUAGGGGAUUUGAA | |
| | | GUUUAAUCUUCUAUCGCAAGGAACUGCCGAUCUUAA | |
| | | UGGAUGGCCGGAGGUGGUAUGGAAGCUAUAAGCGCG | |
| | | GGUGAGAGGGUAAUUAGGCGUGUUCACCUACGCUAC | |
| | | GCUAACGGGCGAUUCUAUAAGAUUGCACAUUGCGUC | |
| | | AACUCAUAAGAUGUCUCAACGGCAUGCGCAACUUGU | |
| | | GAAGUGUCUACUAUCCUUAAACGCAUAUCUCGCACA | |
| | | GUAACUCCCGAAUAUGUCGGCAUCUGAUGUUGCCCG | |
| | | GGCCGAGUUAGUGUUGAGCUCACGGAACUUAUUGUA | |
| | | UGAGUAGUGAUUUGUAAGAGUUGUCAGUUAGCUCG | |
| | | UUCAGGUAAUAGUUGCCCACACAACGUCAAAAUAAG | |
| | | AGAACGGUCGUAACAUAAAGUUCUCCCAUCACAUCA | |
| | | ACCGGUGGCGCAUUGCCACGAAGCAGGCCAAUGGGG | |
| | | AGGACAUCGAUGUCAGACAGAAAUGAAAUAAAUAGU | |
| | | AGUUGCUCUU | |
| R2781_nc- | R2781 ncRNA containing | GGGAUAAUAAGAGCAACUAGAUUGAGGCGAUUCGCC | SEQ ID NO: |
| 04_25 bp_ | 25 bp insertion in EMX1 | UCCUUGGAAAAGGGUACUAAGUUUCUGUCGGAGGAA | 19354 |
| 30HA | gene with 30 bp homology | GGGCCUGAGUCCGAGCAGAAGAAAACUCGAGCUCUG | |
| | arms on each side | AAUGACUCCUAUAAAGUUCUCCCAUCACAUCAACCG | |
| | | GUGGCGCAUUGCGACAGAAAUGAAAUAAAUAGUAGU | |
| | | UGCUCUU | |
| R2781_nc- | R2781 ncRNA containing | GGGAUAAUAAGAGCAACUAGAUUGAGGCGAUUCGCC | SEQ ID NO: |
| 05_205 bp_ | 205 bp insertion in EMX1 | UCCUUGGAAAAGGGUACUAAGUUUCUGUCGGAGGAA | 19355 |
| 30HA | gene with 30 bp homology | GGGCCUGAGUCCGAGCAGAAGAAAAAGCGGUCUUAC | |
| | arms on each side | GGUCAGUCGUAUUCCUUCUCGAGUUCCGUCCAGUUG | |
| | | AGCGUGUCACUCCCAGUGUACCUGCAAGCCGAGAUG | |
| | | GCUGUGCUUGGAGUCAAUCGCAUGUAGGAUGGUCUC | |
| | | CAGACACCGGGGCACCAGUUUUCACGCCUAAAGCAU | |
| | | AAACGACGAGCAGUCAUGAAAGUCUUAGUACUGGAC | |
| | | GUGCCGUUUCACAAAGUUCUCCCAUCACAUCAACCG | |
| | | GUGGCGCAUUGCGACAGAAAUGAAAUAAAUAGUAGU | |
| | | UGCUCUU | |
| R2781 nc- | R2781 ncRNA containing | GGGAUAAUAAGAGCAACUAGAUUGAGGCGAUUCGCC | SEQ ID NO: |
| 06_405 bp_ | 405 bp insertion in EMX1 | UCCUUGGAAAAGGGUACUAAGUUUCUGUCGGAGGAA | 19356 |
| 30HA | gene with 30 bp homology | GGGCCUGAGUCCGAGCAGAAGAACUGCUAAAUCCGC | |
| | arms on each side | GUGAUAGGGGAUUUGAAGUUUAAUCUUCUAUCGCAA | |
| | | GGAACUGCCGAUCUUAAUGGAUGGCCGGAGGUGGUA | |
| | | UGGAAGCUAUAAGCGCGGGUGAGAGGGUAAUUAGGC | |
| | | GUGUUCACCUACGCUACGCUAACGGGCGAUUCUAUA | |
| | | AGAUUGCACAUUGCGUCAACUCAUAAGAUGUCUCAA | |
| | | CGGCAUGCGCAACUUGUGAAGUGUCUACUAUCCUUA | |
| | | AACGCAUAUCUCGCACAGUAACUCCCGAAUAUGUCG | |
| | | GCAUCUGAUGUUGCCCGGGCCGAGUUAGUGUUGAGC | |
| | | UCACGGAACCUAUUGUAUGAGUAGUGAUUUGUAAG | |
| | | AGUUGUCAGUUAGCUCGUUCAGGUAAUAGUUGCCCA | |
| | | CACAACGUCAAAAUAAGAGAACGGUCGUAACAUAAA | |
| | | GUUCUCCCAUCACAUCAACCGGUGGCGCAUUGCGAC | |
| | | AGAAAUGAAAUAAAUAGUAGUUGCUCUU | |
| R1262_nc-46 | R1262 ncRNA containing | GAUAAUGAGCGUGUGACAAGAUUUUGGGCUUGUGU | SEQ ID NO: |
| (ncRNA- | EMX1 deletion (del1) | UUCGCAAGCUUUGAUUAAAAUGGUAGAUGGAUUUGC | 19357 |
| EMX1 site1- | | UAUCUAUCGUCAUUUCCAGUACUGUUAUGUUAACCC | |
| deletion) | | UAUGUAGCCUCAGUCUUCCCAUCAGGCUCUCAGCUC | |
| | | AGCCUGAGUGUUGAGGCCCCAGUGGCUGCUCUGGGU | |
| | | GGGCAACCACAAACCCACGAGGGCAGAGUGCUGCUU | |
| | | GCUGCUGCAUAAUGAGUUGUCACACGCUC | |
| R1262_nc-47 | R1262 ncRNA containing | GAGCGUGUGACAAGAUUUUGGGCUUGUGUUUCGCAA | SEQ ID NO: |
| (ncRNA- | EMX1 deletion (del2) | GCUUUGAUUAAAAUGGUAGAUGGAUUUGCUAUCUA | 19358 |
| EMX1 site2- | | UCGUCAUUUCCAGUACUGUUAUGUCAGAAGAAGAAG | |
| deletion) | | GGCUCCCAUCACAUCAACCGGUGGCGCAUUGCCACG | |
| | | AAGCAGGCCAAUGGGGAGGACAUCGAUGUCACCUCC | |
| | | AAUGACCGCAGCCUCCCAGCUGCUCUCCGUGUCUCC | |
| | | AAUCUCCCUUUUGUUUUGAUGCAGCAUAAUGAGUUG | |
| | | UCACACGCUC | |
| R1262_nc-61 | R1262 ncRNA containing | GGGAUAAUGAGCGUGUGACAAGAUUUUGGGCUUGU | SEQ ID NO: |
| (ncRNA- | sense sequence with | GUUUCGCAAGCUUUGAUUAAAAUGGUAGAUGGAUU | 19359 |
| EMXI sgRNA | tandem sgRNA target for | UGCUAUCUAUCGUCAUUUCCAGUACUGUUAUGUgagu | |
| tandem_sense)- | insertion into EMX1 | ccgagcagaagaagaagggAUAGGAGUCAUUCAACCAUGGGC | |
| 001 | | CCCAUGAUACUGAAAGUGCUGGCAAGCUGCGGGAGC | |
| | | GAGAUACAUACUUCUCGACAGUGGGGCUCAGAGCUC | |
| | | GAGUugaguccgagcagaagaagaagggGCAUAAUGAGUUGUCA | |
| | | CACGCUC | |
| R1262_nc-62 | R1262 ncRNA containing | GGAUAAUGAGCGUGUGACAAGAUUUUGGGCUUGUG | SEQ ID NO: |
| (ncRNA- | antisense sequence with | UUUCGCAAGCUUUGAUUAAAAUGGUAGAUGGAUUU | 19360 |
| EMX1 sgRNA | tandem sgRNA target for | GCUAUCUAUCGUCAUUUCCAGUACUGUUAUGUgagucc | |
| tandem_ | insertion into EMX1 | gagcagaagaagaagggAUAGGAGUCAUUCAACCAUGGGCCC | |
| antisense)-001 | | CAUGAUACUGAAAGUGCUGGCAAGCUGCGGGAGCGA | |
| | | GAUACAUACUUCUCGACAGUGGGGCUCAGAGCUCGA | |
| | | GUugaguccgagcagaagaagaagggGCAUAAUGAGUUGUCACA | |
| | | CGCUC | |
| R1262_nc-63 | R1262 ncRNA containing | GGGAUAAUGAGCGUGUGACAAGAUUUUGGGCUUGU | SEQ ID NO: |
| (ncRNA_NHEJ_ | sense sequence with | GUUUCGCAAGCUUUGAUUAAAAUGGUAGAUGGAUU | 19361 |
| sense)-001 | insertion into EMX1 | UGCUAUCUAUCGUCAUUUCCAGUACUGUUAUGUAUA | |
| | | GGAGUCAUUCAACCAUGGGCCCCAUGAUACUGAAAG | |
| | | UGCUGGCAAGCUGCGGGAGCGAGAUACAUACUUCUC | |
| | | GACAGUGGGGCUCAGAGCUCGAGUuGCAUAAUGAGU | |
| | | UGUCACACGCUC | |
| R1262_nc-64 | R1262 ncRNA containing | GGGAUAAUGAGCGUGUGACAAGAUUUUGGGCUUGU | SEQ ID NO: |
| (ncRNA NHEJ_ | anti sense sequence with | GUUUCGCAAGCUUUGAUUAAAAUGGUAGAUGGAUU | 19362 |
| antisense)- | insertion into EMX1 | UGCUAUCUAUCGUCAUUUCCAGUACUGUUAUGUaAC | |
| 001 | | UCGAGCUCUGAGCCCCACUGUCGAGAAGUAUGUAUC | |
| | | UCGCUCCCGCAGCUUGCCAGCACUUUCAGUAUCAUG | |
| | | GGGCCCAUGGUUGAAUGACUCCUAUGCAUAAUGAGU | |
| | | UGUCACACGCUC | |
| R6342S_nc-63 | R6342S ncRNA containing | GGGAUAAUCAUAGAUUUCUUGGCCUUUAUGCUGUGG | SEQ ID NO: |
| (ncRNA NHEJ_ | sense sequence with | UGUUGCGCCACGGUGGAGAUUUGUCAAAUACACAUC | 19363 |
| antisense)- | insertion into EMX1 | AUUAGGUUGCGaACUCGAGCUCUGAGCCCCACUGUC | |
| 001 | | GAGAAGUAUGUAUCUCGCUCCCGCAGCUUGCCAGCA | |
| | | CUUUCAGUAUCAUGGGGCCCAUGGUUGAAUGACUCC | |
| | | UAUCACAACCAAAUAUAAGAAUUGUUAGCAAGAAAU | |
| | | CUAUGACAUGAGGAUCACCCAUGU | |
| R6342S nc-64 | R6342S ncRNA containing | GGGAUAAUCAUAGAUUUCUUGGCCUUUAUGCUGUGG | SEQ ID NO: |
| (ncRNA NHEJ_ | anti sense sequence with | UGUUGCGCCACGGUGGAGAUUUGUCAAAUACACAUC | 19364 |
| sense)-001 | insertion into EMX1 | AUUAGGUUGCGAUAGGAGUCAUUCAACCAUGGGCCC | |
| | | CAUGAUACUGAAAGUGCUGGCAAGCUGCGGGAGCGA | |
| | | GAUACAUACUUCUCGACAGUGGGGCUCAGAGCUCGA | |
| | | GUuCACAACCAAAUAUAAGAAUUGUUAGCAAGAAAU | |
| | | CUAUGACAUGAGGAUCACCCAUGU | |

What is claimed:

1. A gene editing system comprising one or more delivery vehicles, wherein:
the delivery vehicle(s) comprise RNA cargo,
said RNA cargo comprises (a) at least one mRNA molecule encoding (i) a nucleic acid programmable nuclease and (ii) a retron reverse transcriptase, (b) an engineered retron ncRNA, and (c) guide RNA for the nucleic acid programmable nuclease,
each delivery vehicle contains (a)(i) and/or (a)(ii) and/or (b) and/or (c),
whereby one delivery vehicle or more than one delivery vehicle delivers (a)(i), (a)(ii), (b), and (c).

2. The gene editing system of claim 1,
wherein the engineered retron ncRNA comprises an HDR nucleotide sequence substituted into a retron ncRNA;
wherein the retron reverse transcriptase has an amino acid sequence comprising at least 90% sequence identity to a retron reverse transcriptase of Table A;
wherein the retron ncRNA has about 85% to 98% sequence identity to a retron ncRNA of Table B.

3. The gene editing system of claim 2, wherein the retron ncRNA and the retron reverse transcriptase are from the same clade.

4. The gene editing system of claim 2, wherein the retron ncRNA nucleotide sequence has about 85% to 98% sequence identity to SEQ ID NO:15327, and the retron reverse transcriptase has at least 90% sequence identity to a type I-C retron reverse transcriptase.

5. The gene editing system of claim 4, wherein the retron reverse transcriptase comprises an amino acid sequence at least about 90% identical to SEQ ID NO:1262.

6. The gene editing system of claim 2, wherein the retron ncRNA nucleotide sequence has about 85% to 98% sequence identity to SEQ ID NO:16411, and the retron reverse transcriptase has at least 90% sequence identity to a type III retron reverse transcriptase.

7. The gene editing system of claim 6, wherein the retron reverse transcriptase comprises an amino acid sequence at least about 90% identical to SEQ ID NO:2781.

8. The gene editing system of claim 2, wherein the retron ncRNA nucleotide sequence has about 55% to 90% sequence identity to SEQ ID NO:18731. and the retron reverse transcriptase has at least 90% sequence identity to a type XIII retron reverse transcriptase.

9. The gene editing system of claim 8, wherein the engineered retron ncRNA comprises a nucleotide sequence at least 90% identical to SEQ ID NO:19927 and an HDR template inserted therein.

10. The gene editing system of claim 8, wherein the engineered retron ncRNA comprises a nucleotide sequence at least 90% identical to SEQ ID NO:19928 and an HDR template inserted therein.

11. The gene editing system of claim 8, wherein the retron reverse transcriptase comprises an amino acid sequence at least about 90% identical to SEQ ID NO:6342.

12. The gene editing system of claim 1, wherein the retron reverse transcriptase comprises at least one amino acid substitution that increases processivity and/or fidelity.

13. The gene editing system of claim 12, wherein the retron reverse transcriptase comprises an amino acid substitution in an amino acid residue that corresponds to the following amino acid residues in Eco1 RT: Q190, E302, or T306.

14. The gene editing system of claim 12, wherein the retron reverse transcriptase comprises an amino acid substitution in an amino acid residue that corresponds to the following amino acid substitutions in Eco1 RT: Q190F, E302R, or T306K.

15. A gene editing system comprising one or more delivery vehicles, wherein:
the delivery vehicle(s) comprise RNA cargo,
said RNA cargo comprises (a) at least one mRNA molecule encoding (i) a nucleic acid programmable nuclease and (ii) an engineered retron reverse transcriptase, (b) an engineered retron ncRNA, and (c) guide RNA for the programmable nuclease,
each delivery vehicle contains (a)(i) and/or (a)(ii) and/or (b) and/or (c),
whereby one delivery vehicle or more than one delivery vehicle delivers (a)(i), (a)(ii), (b), and (c), and
wherein the engineered retron reverse transcriptase comprises a processivity enhancing domain or a fidelity enhancing domain.

16. The gene editing system of claim 15, wherein the processivity enhancing domain comprises Sso7d or Sac7d.

17. The gene editing system of claim 15, wherein the fidelity enhancing domain comprises a 3′ to 5′ exonuclease domain.

18. The gene editing system of claim 17, wherein the exonuclease domain comprises POLE1 POLD1, POLG, Pfu, or KOD.

19. The gene editing system of claim 15, wherein the engineered retron ncRNA comprises an HDR nucleotide sequence substituted into a retron ncRNA;
wherein the retron reverse transcriptase has an amino acid sequence comprising at least 90% sequence identity to a retron reverse transcriptase of Table A;
wherein the retron ncRNA has about 85% to 98% sequence identity to a retron ncRNA of Table B.

20. The gene editing system of claim 15, wherein the retron ncRNA and the retron reverse transcriptase are from the same clade.

21. A gene editing system comprising one or more delivery vehicles, wherein:
the delivery vehicle(s) comprise RNA cargo,
said RNA cargo comprises (a) at least one mRNA molecule encoding (i) a nucleic acid programmable nuclease and (ii) an engineered reverse transcriptase, (b) an engineered retron ncRNA, and (c) guide RNA for the programmable nuclease,
each delivery vehicle contains (a)(i) and/or (a)(ii) and/or (b) and/or (c),
whereby one delivery vehicle or more than one delivery vehicle delivers (a)(i), (a)(ii), (b), and (c), and
wherein the engineered reverse transcriptase comprises a Y region domain that is from a retron RT that corresponds to the engineered retron ncRNA.

22. The gene editing system of claim 21, wherein the engineered reverse transcriptase is a chimera comprising an MMLV RT fused to the Y region of the retron RT.

23. The gene editing system of claim 1, wherein (a)(i) and (a)(ii) comprise a single mRNA molecule encoding the nucleic acid programmable nuclease and the retron reverse transcriptase.

24. The gene editing system of claim 23, wherein (a)(i) and (a)(ii) are encoded and expressed as a fusion protein.

25. The gene editing system of claim 24, wherein the fusion protein comprises the C-terminal end of the nucleic acid programmable nuclease fused to the N-terminal end of the retron reverse transcriptase (nuclease:RT fusion).

26. The gene editing system of claim 24, wherein the fusion protein comprises the N-terminal end of the nucleic acid programmable nuclease fused to the C-terminal end of the retron reverse transcriptase (RT:nuclease fusion).

27. The gene editing system of claim 1, wherein (a)(i) and (a)(ii) comprise a first mRNA molecule encoding the nucleic acid programmable nuclease and a second mRNA molecule encoding the retron reverse transcriptase.

28. The gene editing system of claim 1, wherein (c) is separate from (a)(i), (a)(ii) and (b) or is provided in trans.

29. The gene editing system of claim 1, wherein (b) the engineered retron ncRNA, and (c) the guide RNA are fused or are provided in cis.

30. The gene editing system of claim 29, wherein the guide RNA is fused to the 5′ end of the retron ncRNA.

31. The gene editing system of claim 29, wherein the guide RNA is fused to the 3′ end of the retron ncRNA.

32. The gene editing system of claim 29, wherein the engineered ncRNA comprises a first guide RNA fused to the 5′ end of the retron ncRNA, and a second guide RNA fused to the 3′ end of the retron ncRNA, and the first and second guide RNAs target different sequences.

33. The gene editing system of claim 1, wherein the one or more delivery vehicles comprise a liposome or a lipid nanoparticle (LNP).

34. The gene editing system of claim 1, wherein (a) the at least one mRNA molecule encoding (i) the nucleic acid programmable nuclease and (ii) the retron reverse transcriptase, and (b) the engineered retron ncRNA are in the same delivery vehicle.

35. The gene editing system of claim 1, wherein the (a) the at least one mRNA molecule encoding (i) the nucleic acid programmable nuclease and (ii) the retron reverse transcriptase, and (b) the engineered retron ncRNA are in separate delivery vehicles.

36. The gene editing system of claim 1, wherein the nucleic acid programmable nuclease and the retron reverse transcriptase are encoded on separate mRNA molecules and those separate mRNA molecules of (a)(i) and (a)(ii) are contained in the same delivery vehicle.

37. The gene editing system of claim 1, wherein the nucleic acid programmable nuclease and the retron reverse transcriptase are encoded on separate mRNA molecules and those separate mRNA molecules of (a)(i) and (a)(ii) are contained in different delivery vehicles.

38. The gene editing system of claim 1, wherein the engineered retron ncRNA includes a sequence of interest encoding a donor polynucleotide comprising an intended edit to be integrated at a target sequence in a cell, and wherein the donor polynucleotide is flanked by a 5′ homology arm that hybridizes to a sequence 5′ to the target sequence and a 3′ homology arm that hybridizes to a sequence 3′ to the target sequence.

39. The gene editing system of claim 1, wherein the nucleic acid programmable nuclease comprises a Cas9 nuclease, a TnpB nuclease, or a Cas12a nuclease.

40. The gene editing system of claim 1, wherein the nucleic acid programmable nuclease comprises a Cas9 nuclease.

41. The gene editing system of claim 1, wherein the nucleic acid programmable nuclease comprises a Cas9 nickase.

42. An isolated cell comprising the gene editing system of claim 1.

43. The isolated cell of claim 42, wherein the isolated cell is a mammalian cell.

44. The isolated cell of claim 43, wherein the mammalian cell is a human cell.

45. A composition comprising:
a) the gene editing system of claim 1; and
b) a pharmaceutically or veterinarily acceptable carrier.

46. The composition of claim 45, wherein the delivery vehicle is a lipid nanoparticle comprising:
a) one or more ionizable lipids;
b) one or more structural lipids;
c) one or more PEGylated lipids; and
d) one or more phospholipids.

47. The composition of claim 46, wherein the one or more ionizable lipids comprises an ionizable lipid set forth in Table 2.

48. A method of genetically modifying a cell comprising:
contacting the gene editing system of claim 1 with the cell, thereby delivering the RNA cargo to the cell,
wherein:
the nucleic acid programmable nuclease forms a complex with the guide RNA, wherein said guide RNA directs the complex to the target sequence,
the nucleic acid programmable nuclease creates a double-stranded break in in the target sequence,
the retron reverse transcriptase and engineered retron ncRNA create RT DNA that comprises the donor polynucleotide, and
the donor polynucleotide becomes integrated at the target sequence,
whereby editing the cell is genetically modified.
